SEQUENCE LISTING (42 sequences)

SEQ ID NO: 1 - DNA, 36676 bp, Homo sapiens
  source 1..36676: mol_type = genomic DNA
ttcctggtgtaagctttggtatggatggtggccgtctccctacagactgggagctgttagagggcagggatcctagctgacacatctatgtcctcgccttggttggaggtaggtatgggtggcggcgagggaggagatgggggtggggctcaacacactgtccagcccctgtcgtctggctgggtatcgccagtgctcaggggcgagcggaatggagaggaggcggggctggggcgcacagccggcaggggatccctgccggagtctgcttttcacgggttcccgtctagaccagagttggggctcagcttggagagatgggacaggtggaggtgctgggggttggagccccacgcgctgctcaagaggctgcaggctggccttcaaggcctgggctagactgcggcggttgggggctggcggtgcggctcgggtggagtccgccgggcaccgccctccctctccaccctcgcccaccccggggccgcgaggctgtgggttgagctgcagaggcgagctccctcctgggcgtgccgccgccgcgagtggggtgggagcgcagagggggcctcggggttatgctttgctgggatctgggaaggaagcaggtggcagcgccggagtgagtaaccgcctcccgaggcagctagtcccgccgcctcaggaagtcctggcgccgtagcgcaccgccgcccgccacaccgggaccttcccctcgcactagcccgcccccaccccgcctccatctcgcgctcccagccgcagacacgggcgcctctgcactctcaacccctcacccacgcagaccctggcccgccggtgacctcccctgcggctcctccttctgccccggcccagcccctcccctgctgctcctcctcctgccccggcccagcccgtccaccgtcctctcatccccatggcctcgaaccccagcctgccgagccgctcctcacagggtgctgtggctccccgctgcaaaggggcgggtcctggtagggggcgtggcctggccgacttgcggggcggcggcgccctctgccggcagactcgggaacggccacactcgtcacctcgcccccggcccggctcctggacccccctggacctctctgccagctctcctctctcctggaaatgcgcctgcaggactcggaacgtgcagaggtcggtcctggccccagccgtgccacgggctatctgagggagagtcgcggcctctctgaggcttggcttcctttcctgtgaaatggctgtggtgaggagcaaacgctcagcagtgaagaaggaccctttgtaaccgcgaaggagcctgtgagtgtgaggtatggttgttatcaacgctaagggcagtcttttgccttctgcgggggtgacgaccatccttctccctgccccagccccacagtggaaggaaaacataagtcacctccagagattatcacatgcagtggattgctttcagccttttttctgaatataaaatatacatatacgtattttaatacaattgtggcccttctctacgtgctgttctgtgctgcctttttttctctctctccaagtgcttaacttatgttttaaagcccagattcggccagaggggtggctcaccaacactttgggaggccaaggccggaggatctcttgagcagaagttcaagaccaacctgagcaacatagcaagacctcgtctctacaaaaaatttttaaaaatgatctgagtgtctgtagtcccagctactggagaggctgagatgggaggctcgcttgaatcctggagttccaggctgcagtgagccgtgattgtgccactggccactgcactccagcctgggtgacagagtgagatcctgtcacaagaaagaaagaagaagaagaaaagaaagaaagaaaaagaaaacaaaaaaccaaaaagaaaagaaacaaccaaaaaactagagtcagacaaacctggatttaggttcttgctctgtgactttatatttccacccttgagcaaaccacacaacctctaccttcctcagcctcagtttcctcctttgcaaaatggtggtaatgccaggaatacccacttcacaggcagcggtgaggccccgcacacagcccagtgcatgctggttagggctctccttttgcttcctgagactgcacgagctctgccttcttcatgcaccacgtggtaaggaaggagctggaggcctccgcctctcggaccttgctcactgctgcttttttttttttttttttgacagagtctctctgtcgcccaggctggagtgcaatggcacaaccccggctcactgcaccctccgtcccccgggttcaagtgattctcctgcctcagtctcccgagtagctgggattccaggcggccgccaccacgcccggctaatttgtcatatttttagtagagatggggtttcgccatgttggccaggctggtctcgaactcctgacctcaggtgatccacctgcctcggcctcccaaactgctgggattacaggggtgagccaccgccccaggcctctttcggcactgccctcaaccacatgggcctcactgttcttccggcgtacccagactttccccatctcagcgccttcccaccggtcctgtgtttccatggcctttccccgggtaggggcctttcttttcctagcttcgctcagttccctgctacattgtcgcctcctcgcagaagccgtctctggccgttccgtatcgctagcgccctctatcattcgctagcagctcacccggctctgtttttcttcattgcacttctcgttatgtgccgttctcttataaatgtttttgtctttatccctcccatagaagacacctccatgaaaggccagggctttccgaggttcttttcctctgtggttcctagaagagggcctggcacatagccggccttggctagctttttgcgtaaatgtgaagggatccaccttcctcccttataagaggtagcagtacctcctgttaccagcagagggcaccaccgtacagcacttgggggccgcaggggatcctgctgggatgggaggtatttaaaaagcccacaggcctctcctcccataatgtgggcgctcccacagctgggaccggaagcaggagggccccctgcgctaatctgccctggcagcagcgagctggcacgccctgggtgcatcatccggtgtgctctggtgcccaaggtcggaataccctcactggtggcttggacacacccctgagcagctgatgcttctggacagatcacaccctgtcccgggctctcaaaatgagggcaatgacatgagctgcactgctcgctcacgcgtttgctgtgaggttgggacaggagtgcatgggaaatgttttatgaatgtaaacatgggacaaatataatttatgcacgcctttattggtagtgcagcagattgacaacaaacgaggggagaagataaggtatttaaaggaaattgcaaggctaaatgaaggtgggaattaagacagtgaaggagaacgaagaagttaaaaataacaactacctttcaccaagagcttattaggagccaaggcagaggaaagtgatctccaaacattgtctaattatagccaacacttacatggtgctgacctcatgccaggaactctttaagtgctttatgcataaaaactcactggatcctcacaacaactcagggaggtaggtcatatcgctgtcccgttctacacaaggaaactgaggcccggacagcttaagtgactggcccaaggccacatagatagacagggctgggcaatctgcctcctggaatgtgctcttaaagccccacagtcacagccacaccaaaagggagatattgttattatcattccatttcccaaacaagcaactgaggcttagcagatgtacaaagcccgcctaaagccacactgggaatatgtgtatttgagcccagcttaaccacccaccaaactaggcctgcaggagctaggaggggcttcaggaatgtgcacaggttctctcggagggcagcagcagacatacccactcttcctctgagcggggaggggaaggtgagagcacagaggctgggcagggctgatggggttgaggaagaggctgcttggtggcctcaactccatggaccgcagatggccaggcatcttctggccaggaagtgagaggtgtgttgataggcacagtggctgtccacagggattccagccagagctgtgggccccccacaaatctccatagggagactagggactgaggggtgggccttacagtgggaggtgggagcagccccatccctaccacaagtgccccttagcccagccctgacttccagcccgttacataagctgctgcatacccaggcctggaaatagctgaggctgtaccggctgggctgggagacgtgatgaatggtccccaggagcatcagctgaccgccctgtgctcctgaagtgacctgagggcctgccagcagctcgggttacccagctctccctgggaggcagccctgcctggtctgctgaggatggctctgatgtgattctgtgtctggacatttatggttcgagtgcccgcaggatggtccctctcccaagcctgagcagctaaggcagctcccagccacgctgtgcccactggcagcctctggatgctgggagccagggccttgcccagagcaagcacttggatctctgcagacagtcagcactgggagggctgagagctctgctgctctgagtcctgtgctaagggcccgcaaggaaagaggaagaggagcctgagaaactcggggtgcacgtgggggctgcaggtgtcattccagggctgcactccacccacatccccatgcctcctgtttcacggcccactctgttactgaactggtggccctgggcacaggggggcacccacacactctgatgcccactgcagagggcagagccacacacccctccagggactaggcacatacacccttacttctgagagtaacgtggctaggctcaggagtgtaggagggcgttctcccagcatatggtgatcatatgttaagaattattattatgttggtcaggtgcggtggctcacacctgtaattctagcacttttagaggctgaggtgggaggattgcttgagtccaggagtttgagaccagcctgggcaacatgatgaaatcctgtctctacaaaaaatacaaaaattagccgggcgtggtggcatgtgcctttagtcccagctactcgggagtttgagatgggaggatcgcttgagcccaggaggttgaggctgcagtgagccaagattgtgccactacactccagtctgggtgacaaagtgagaccctgtctccaaaaaaaaaaaaaaaaaaaaaaaaaaagaagagagaagtatgaatgattatggccatgccaattctgtgcctttaagatacctgcttaaggggccgggagcagtggctcacgcctgtaatcccagcactttgggaggctgatgcaggcagatcatgaggtcaagtgatcgagaccatcctggccaacatagtgaaaccccgtctttactaaaaatacaaaaattagctgggcgtggtggctcatgcctgtagtcccagcaacttgggaggctgaggcaggagacttgcttgaacccaggaggcggaggttgcagtgagctgagattgagccactgcactccagcctcgtgacagagcaagactccgtctcaaacaaaacaaaaaaacaaaaaagatacctgcttaaaagacaaaaagaccaggccaggcgcggtagctcatgcctgtaatcccagcactttgggaggccgaggtgggtggattacgaggtcaggagttcaagaccagcctggccaagatggtgaaaccccgtctctactaaaaatacaaaaattagctgggcgtggtggtgggcgcttgtaatcccagctacttgggaggctgaagcagagaattacttgaacccgggaagcggaggttgcagtgagccaagatggtgccactgcactccagcctgggtgacaaagcgagactccattttaaaaaacaaaaacaaaaacaaaaacaaaaagaccaaaattcaacttctccctaaaaatattaatgctttaagtgaaattatctctctagtattccccaaatcctttagtggaataggcataataggcactcccagggagctaggacctgtttactctgattccaggaccttctgacaggtgggaagcccccagctggagaagtgtgggacattttctaaaagagcagtggaaagtgagtgagttgccccatctgcctgaccacagattgcctgtccaatgacaggagggacgcatacatgcacacacactccacctctctccttggtgctcagatgtgggggcagaattcccctccaaacgcctgcagcagcattccctgaagaccctgcctgtgcccagaccctgcccatgactttgcaccctccttgtgtgcctgtgggtcctgcagttctgagcccatggccaaggcacaggggaaacaggcccctaatgtactccagagacggccggtgtggtggcccacacctgtaatccaacattttgggaggctgaggtgggtggattgcttgagctcaggagtttgagaccagcctgggcaacagagtgaaaccccgtctctacaaaacatacaaaaattagccaggcatggtggcatacacctgtagtcccagctacttgggaggcaaaagtgggaggatcacttgagccagggaggctgaggctgcaatgagctatggttgcatcactgcattcaagcctgggcaacagaatgagaccctgtctcaaaaaacaaaaaccacacacacacaagacagaacaccagagagctaaggaatacctacccctctgccatataaacctgtgtggggaagggcttggcttccttcagttccctcccgcctgcctcatgtctctccaggcactctcccttgagggttgtcaccggtaaccagagctggctcccctggtgagagcaggagcagcgttacttagcaacaggcacagcacacaaaatccagctaatctgggctgccccagtggccagccctcctacagccctcagggcggtggctgctggccagcagcacacccctagaagtggacacttataaacttctgcatctttctaccccagcaaaccctaagttccagcttgggcaagaaggactcttgcacacaaaggcacaaagacatgggcagggtgtgggcacaggcaaggtcttcagggaatgctgctgcaggggcattttctccgccctccctgtccctctccccttccttaggccctgtctcccttcacagtatctacagaactctctgaggcgggggtcccagctctgcagcaagccatgaaatgaagggcaaatcccttcacctctgggtagctcaacttctctgggaagcaataacatagcagctcagtgtaaggactttgaggccacactggtctggatgcaaatccatgctctgctacttctcagctgtgtgacactggacatgtttacttaaccttttatttattttttattttgtattattttttgagacggagtcttgctctgctgcccaggttggagtgtagtggtgcaatcttcgctaactgcaacctccacctctcaggttcaagtggttctcctgcctcagcctcctgagtagctgggattacaggcataggccaccatgctgggctaatttttgtatttttagtagagacggggtttcattatgttggccagtctggtctcgaactcctgacctcatgtgatccgcctgccttggcctcccaaagtgctgggattacaggcgcgagccaccacacctggcctacttaaccttttagagcctcagtttcctcaatactaaaatagtgcccaccttagatggctgctgaaagaagagacaaaggaaaaaagtcatcggcacagagtctggcacaagggcctgtggattgggctgagtgctcctctcgcgcatccccagctctgtcccccttagtcactggcctccagccacttgccttactctgctgctctaacaagctggcactctttcattactgtttcctctccctgcaacttatccctttcccctaactctccaccgacaaactcccctcagtttatttattttgagatagggtctcactctgtcgcccaggctggagtgcagtggtgctgtcacagctcgctacagcctcagactcctgggctcaagtgattctctcacctccgccctgaagtagctgggaccatagtcccgtaacaccaagcaggctattttttaattattattattacttttagtagaaacaaagtctcccttacgttgcccaggctggtctcaaactcctgagctcaaatgatcctcccatctcggcctcccaaagtgctgagattacaggcatgagcctccagtggtgcccagccttccctcagtttaaaagctacttcccgccaggcacagtggctcacgcctgtaatctcaacactttgggaggccgaggcgggcagatcacatgaggtcaggagtgtgagaccagcctggcccacatggtgaaaccccatctctactaaaaatacaaaaattagccaggtttggtggcatgcgcctgtaatcccagctactcatgaggctgaggtaggagaatggcttgaacctgggaggcagaggttgcagtgagccgagatcatgctactgcactccagcctgggtgacagagtgagactccgtctcaaaacaaacaaacaaacaaataaaaaagctacttccttggagaagctttctcttatccatcccttgcgaaaattacagctgcctgttaaatatctcactgacatcctccatttcattttctctttatggttgtgatggttaatttttttttttttttttttgagacggagtctcgctctgttgcccaggctggagcgcagtgacgcaatctcagctcactgcaaccttcggctcactgcaatttccgtctcctgggttcaagcaattctcctacctcagcctccggagtagctgggattacaggtgcccgccaccacgcccagctaattttgtatttttagtagagacggggtttcaccatgttggtcaggcaggtctggaactcccggcctcaagtgatccgcccgcctcggcctcccaaagtgctgggattacaggcttgagccaccgtgatggttaattttaagtgtcaacttgactgggctaagggatgctcaaatagctggtaaaatgttatttctaggtgtatttgtgaggctgtttctggaagagattagtatttgaatcagtagactgagtaaagaagaccgccctcaccgctatgggcaggcataatttaatctgttaaggacctgaatagaataaaaaggcagaggaagggcaaatttgctcttctttgaggtgggacaccatattttccctgcttcagacatccatgctctgattcttgcaccttcagacgcggactggaactactccagcttgcggagggcagatcctgggtacttctcagcgtccataatcatgggagctaatgcctcataataaatctttttctatatctctgtatatccatcctattggttctgtttctctggagaacgctggctaacaccatggcacttactgcttatgtgcaagattgttgaacattcatctctcctacttggctgtcagcccggggacaggagctgtgtctgttttgttctgtgctgtgctgtgtttccagtgagagggctggcctaggtgtttctccgaagtatgttccagctttcggtacctctttatttcttatttcttccttcccctgctctccaagttgacctaaatagtttttgcaccttttcttaggttcactgaatctccaagcagcgaggtgtatctggaaagacagctcaagaattaagggtgctcacccttaattacactccattttcctcaggacaggctggaacagatttgggatggcgagcccccaaaccagcactctacagatttcgggacccctcttaactcatttcccccatcctcccaatgacgtctattcgcctgcagcagacccgaaggaagcttcggctcaatggttcatttgcatgcgcccgtgctattggtcggaggggcggcaccgtagccaatggtgtgcaggggtgttgaccgagccgcagctctgcgccgccgggtgaggtaatggaatcctggctggacctgctgcggctccctccggactggctagtgacgtagcggcttccccggccccaccccgcccagcccggccgcccccgggagcgcagctggggctcggccctgttccttccggcccggcgttccggtcgcgggcccctcagggaggacccgccgggccagatcccatctctggcgacgcggagaaaaatctggaagccgtcgtcggaggatgaccaaatcctcgctcctctgggagtgcgattacgggcctctcacttcgtaaatcacgcaggcctgaaacgctcacatttgtttaccacgagcatgtttgattttgtaatttaacaaataaaattgcagaggatttttaagagccctggcctggatttaggggaaatctcgcattaattccagctctgccatttccggtctggagaccttggccactttctcttggggccctgacgccttccatggcaactaagtagagcaaacgtggaagtgcttcgaaaacaccaagtggggtctcctcatccttatgcaatctggagagtgtaaccccatctgggcggtgtctctggacagcccctgagccctgagacagccccaggaagacccgtctttgtgtctctattttttcggcccttaaattgctcttaagcctggggagaggtcgggggaccccggccaggtagttcttgatcctcaagaagaggaaggaaaccatgaagggaatcccttccacagcccctctccttattcagggccagggcctgagcaggggagccccttctctgcccaaatgccctagggaacccccttgacatccttttacccccgcccccgagggccaggatggaggtggagtggaagggtgtctcctaggtggggtggagtttcagatgctgtgagatggaagggcacctggggagggccactgagagtctggcttatggtcctgggtcctgtggcttaccttggcccactacttcccaccctcaggatctcagccctgccctgccgcatacctaaggtttgggggcagcctctctcctccatggagtgccttttctctataccctgcctggtcctgtctcttctttcacactgggccctacattctcttcctccagggaggcctcttgagactagtcctctagattctaccccaatgccagctcaaggcttcttggaggccacttcttggatgtcctggcaatgtttccatacctattcttgtgtcctgcctccccatcagactaggagccagctttctgtgtaggagcttgtcctctaggaacctcccacaatgtggggcgtcactggccatggtggcctgtctcccctctgctccgactccctgggccatgcacgttgttcaccttttctctcctcacctttcctatctgcaataggggctgtgaagcagtgcctgtagaaaggagaggaattagatatgaaggaacaagaggctgggtagaaagagcactggcctgggagtcagaagaccagggctatggagacagaagaccagggctatggttctggctgggcaactcactagctgagtgacctggatcagggagacaacaacagggcagacgtttactggacatttgtaatgactaagaagcgacattgctcctatctgtgcagcccccacggcccccagtattcccaataaacatactggctgggcaaagctagggtaagcaccttggatgtacagcttcaagacaaggtcccaagtcctccaagtcctcctgtatggaaatggcagatctgccatgatgtgccagactccgcttcatgctttatgtctgctctcaaagaggcactctcatggcaaccctgtgagataggtttcttatcatctttgtgtttgaggtgaggaaactgaagcccagagagggcaagtaacttccctggagtcatgcagctggtaagaggtagagcctcttgtgggttagggaaatgagactcctagggcagagcagggaggagaactaactctgagtgcctgctgtgtacaaggtacctccacatctgttcagtcctccgacaaccagtgtccatcccagacctctagagactgcttcttccaccatctcacgccgcctccacatgccacccaaagcgtgcgtgttctggcattccacattttagaatatgttttatttgattataaataggagaatatataataaagacaagttatgctaaaagtatttatggaaaggagagacctctgtgggctggaagccttcatggaaaaactggtaactgagccctttttttcccttcctgtctaaataaatgtatttctcccacacaggcaaatattttaaaatgtatttcaaaagttagacttctggaaaaagaaaggaaaagagggggtggggtagctgctcctctaagaagcccatgtgaaagaagctgctggggtaggaaacaggagggcacagggtgagtgaggaaaagtcagtccctttcccccaccccaacgccttgtcatttttgaagcctcagtttctttatctgcaaattgacctaatacttgaacctgggagagaaatgtacaggttcttgggatgtgattttgagagacaaactctgcagaggttcttggaatcattatgaagaatgagagaaagggaattctgagttgcgccaagagtttaggatttgtccagcaaaggatttctgggggtgggcagtggggtggagagaggaatgcagcccctggaatgagggctgagtggaggtgtggggcctcttttggagaattttgagcacagaagagattctttgctgcgtggggcctgggctgggtgctggagattgtatagcatgctcataatttaaggtggctccaaaaaggatcaggggctagtggctggcacagcagagcacatgactggatcctccctaatgggtgttccccaatgaccttgcctgtacgggggtataggagcagagatggtaacccccaaccccacccagccagccccaatcccattgctccaactttggccgggccttcctgaccagcctctcctgagtgggcagctgctgggggagaaggatttgctcaggacaggatggttcttccatttccaaagagggctccagtcacatgggagagagaaaccttccaagtgcctgcacccccaggaaaattcttaccatggaagcctgggcctgaagactctggtcagaggagggtgggcttcccagggcttcctggtcacggtggtgggcctctgagtgcatgaagcactcctttggattggggagagcagggagaactaactcagtatctactgtgtacaaggcacctccacagctgttatcatcctgacagccagatatggtggataatctgatcatatcatgttttagagacgaggaagctgaggctcaggaggtcaagtgactgtcctaagtcacacagtggtatgggagcagccagatcagtgaatccatcacctgggctttgccggggcctgcacccccgcctcgggaagtgccctcttcaggagaaagtcctatttctctgtaagctgccttctcgtgtggaagctgggtgccctgctacctgggttccttgcccttggggtggcatgaatgccaagcgatgcaggcagggcgcccgcagactcaaaggccaggccctgtcctcgccagcagccgaatcgctgtgactttggaccgccaccgagtctctctgggcctcgctctttccacctcttcctggggaagccccggctaagtttctttataacctagccccaggtgcccagcctcggaaggtgtgagcgagacctcggcaccctcacctgagaaccagctccccggctctggccgcgggttatgtaaccgggtgtgcggcgtggggggagccgcggggcggggcgctcgcggctattccgggcgccgctatttttagccccattgatgaggctgcgtcggccgccgccgccgggattccggagacgtcaatgggcacgcgtcacggtccccgccccgcccgccgggggaggcgcgccggggctgggacagcgcgcgggcggccgcgggcgcgggggcggggggcgccgcgcggccggaactgcgggcggggggcttcccgggggcggggggcgctgagcggctgcggcgcggctgcaggagcgcgaggaggaggtggcggcggcggcggaactggcgggggtcccctcgcggcgcccgggacccccggcctccaggcatgggcgcccctgcggtgcagagcagctcagggccggctggtgcgcgaccccggaaagcgggggtggagaggcgagcggagcccgcggggccaggtgaggggctgccggggtggcgggaggtgggccccggagaggccgggcaatctcgggtcttcggtggatgcctttgggcgggctcggggccacgccggagtcccgcttggaaactggggagtcgggggggggactggattcccagcggaaccgctggctccgagtcagcccccaagggcgacggccaaggctttctctccccagtccgccaagggtggggtttggcccacgaggggcgctggaggtggccaggccagagaaattccttccatttctccgccggctgcgggaaccggttccaggagcgcgggagctcgtctgccaggagacccagccgggagggaccgatgctgcgtgatgcaaccccttcgagggactgagtcaagaaaccaaggctgggggtctcgtggagtcaggcggagctggggattaggactcggggcttgtgtctcccaggcgtgtttttttgttgttgttttgtttttttgccgcaacatcttccttatactgggtggggtgggggcgggggtggtctgtatttccagacctcctccatggtggtctggctgtgtggagggcagggcacagttccttgacttggctcattcccccccaacgccccccctttcttttttttttgagatggagtctcgctctgttgcccaggctggagtgcagtagcgtgatctcggctcactgcaatctccgtctcccgggttcaagcgaatctcctgcctcagcctccagagtagctgggactacaggcacccaccaccacgcccggctaatttttgtatttttagtagagacagggtttcaccatattggccaggctggtctggaactcctgaccttgtgatccgcctgcctcggcctcctaaagtgctgggattacaggcgtgagccaccgcgcctggcaggctcattccctttttatgccttcgtttttgatagatgacctggggatgggagaaatgctgcagaggtggtttatttacagtgaattgttaatttggttgtggggctagtattaggggcacattgggaaaacacaggctgggcagaggttggtaggttcacaacagttatgcccactgggtgctgatggatttgggggctgtcgtcggcctggcttggggccctagtggtggggctacaaggcttgctcctagatctactccaagtgaacatttaagatcagtgacttggatggagacttaaaaaggaagtcagctgactccgtgtagtcatccagagctgtgaggaatgacaagtgcacagtataaaatcaagattctaattgaaggtaactgggcagaaggatgggccgagattaacggtgtgaatgtgactgacgattcctgtgctgaggcctctgctggacttaagaaagtcagctgaggagttcaggccctgtgcctggctcaaaggcccagggacccaggcccttagcttctgccctgctctctggatggaggctgtgggactgctctgtgcagtgtgaagcctccatccgctacacaggtccaagtgggtggcagggggagcgagaatgcctgattctggactccttggttaggctccctgggttctaatcccagttttgccaattcctagctgtgtgaccttgggcaagttgcttaacatttctttctgggccttagtttcctcattggtaaaatgtgcataataataatcccttcctcataacattgtgaggctgaaatgagaaaatgcatgtaaagtgcttaatgtatctggcacatgttgtttaacaaatattagctgctgttgttattgctaataatagtaatatgcccacttctgggtagggatattggccaatgcagagcttgtactcaggaggccaccaggacctgtcacgcagagggcctgtcagcggtgtcctgttgcaaggggcagtttattaagaaaagactaaaatacttgctaggaaggactagaatgaggcttcctgtgggccagtgcaggggacagagctgggatccagggctggggacttttagagtcagggatgtgattcaacataaggaagaacctctgctctggctcttggatctgccgccaaatggaagggacagctcctgagctggaatgctctccccacctagctgaccttcctcccagcacagctccaatgcaggcatgtgcatccaccacccttccacgcacacttactgagcacctgccagtgctgagcagggtttagagactgcagactctgcagtgattgagatggacagcgtccttagattctggttgggtagacaggtgataaaccagtgaacagatgtgaacattagacctatgatgaataaaatcaggctagtgtggtggggtggggatgcaagctgttcagggaagtgacatgggaatggaaacctggttgatgagaaagagccagttctgggcagagggaatggcagatgcagaggccctgagtggggagagaaggcccacgtgatgggaccatagtgagtggctgggaaatgaggttggtggggagcaggagctggggactgaagtctggcctgatggtccaccccattttgggggccactttctcctcctccaagcccactcagcctttggtttgctctctgcacagtgcagactgtacttgaccttggttcatcccttatcagctcctggggctctgggatcttggtgtgctcattcttgcctctcctgttgttcggggcacactgcctggctcccgggggtcctcagtaaatgcttgtgatgtgactattgaggggggcattttgatctggaacaagtggggaaacagatacctgtttgaagatgctatgaatcatttgtgggcaccacctggtgccaaactctggcccccgttctgagaaccagatggggaagacctgggcactcaggttgggcctgactatggtaggtggtatttggccctccctacccttcctcctcagcagtggggtggtagggagaaaagacaaagctgggggaaactacagaccagtttcggggggatgggaggatgacactgtcatcctggaaagcaggtgtagtccatgggccggggcaggttggaaaccttgtcttgctttctgtttggggttaaaggtgctccagatgttccagtctgccctcccttccctccttgtctcctgtggccttttcctgcttcccatcatacccacaggatcgggagggaggggtgctgacaatgttgtaaggggagagcctgggtactgtgggccagaggatcttcccttggcctgtagaccttgggcaaaggtcaggtgggtgaggaagcccattgcccaccctctggggtcctcctcctctttgcccctcctagctcccatcatctccttctcttttcccagacctcatctcagtctgaagggcagtccaatagccagtgaaggcctggtcctgccctcactctggtgcgtagtcgagaggagagagcactggaggggatggaagaagagaaggggagatgtgggtcaaggtgccgtggctgaatggggttgggggagagctgaagaggctggggtgtggggtgaggcacacagttggcttccaccctggaaggagaggccaggtgcccagggtaccttggactaggaaccaggggttggggctagcttttatgtgtgtgtcctcattgaacttctcctaatattgatatgtagtagatatgtgtatccagcactgtccttaagtcttttacacattaacttttttttaagcctgctgataagcctgtgattgttaattttacagatgtcatcatttttacagatgaggagcatgaggtacaaagagattatgtaatttgcccaaggtgataagcagtggagctaggattcgaacccagacggtctgggatcccacatccactctactctaagttgagtggagcccctgtctcttgggggcctccgaggctttaggggccaaactggagccccttaaggagagtggggtattccgctctgtcctcacaatagctccctttctgtgcccgcagcatgcccttttgtgcatgtcaaaggccgagagccaggaagggtatgaggtggagttgctagatgctgatgtgtctatatggctctgaaatgccccagatgctatcagcagctcctggccacaccctacacccctggaagcagattgcctggggcctcggcccccaggctccaatgcctaacccggggaggtccttgggcgctggcttgtccccctcttgtggcctaggtcctgccactgctcccattcctgcgtgtcctgggcatgctgtctccagggaatgtgcctgctggctggtttctcttcactcacatcgactctccctctgtaggcctccaccatggacagaggccaggccctgcccctcccaggcagcctggctccttctgctgggccctgaaggcagacgggataatgtggttggccaaggcctgttggtccatccagagtggtaagtgctctgctattgtcctttgtacacccctccagcaggtggggttggggggcagaggtgggggtggagctgcccaaagcggatgtggttagggggatggaggggaagccctggggaggtggcagccgaggtgctgttcagtgtgcgggatctgatttctttagaggttggggtagtgtgagtgtggggactggggaagccttattttcttggcaggataggctggagggctctctgggcctaggtgacacgggctggtgggagtaggggatgacagctgctcccaaggcagcatgtgcctgattccaggaagcagatagggtagaatagggtgtctgggggctgggctgggagcatcatggggcagacgtggcagcggggaactggcctgtctcccaggtgtagacacttctccaggtgagtgcatgaagggaaggctgggacctccccttcctcgtggaaagcatggtgcctgcccctcctcactgtggcaggccagagggcctatggagtctgtggtgtcgggggtggcctctgtgaatgggaaggcagggacagcccttccactactggtgttctagcacatctccttcacttacagaagaggagacttaggtacagaggggagaaatgacttttccaggggtcctgcagtgaggatgtgtagagccaagcccagaaccatgcctcctgggtggctggcaagcacaggcacctggtctgactgtggaagacataccttccttgactgagggtgcttccggagaaggcccacactgatctggggctgagaggtggaggacgggaatgggattttcagcaggcctttcctggggacatgagatggggccaagggcactgtagtcctggttctgtgcctaaaggagctggagctctgccttgcaccccagacaggggtcatgagattcccttcctccttccctggaagagggccatctttgggcagcagccctcccaggccttgagaaatcccggatgaagcacgcactggctcagtatctccctcagcttcccaaacactgttgcctcatggggggtcagcgttcagggtgcagagagccccagagaggtggctggggtggggaggatggggtgtctggtcccagcctggagggaagggggaagccctgggaagcagggcctcctgttctgggggcagggctgggtgtgtgcctggcctcctgatggcagcaggtgctgtaccaccaacctcgggctgaagacagggagaggaattactgtaagggggagaggcaggtgttctctggtcagctggagatctggacttgggaagcacgtgagtgaggatgacagcagtggccaggtgtccctccctcaactcggcccctacatccgcccccagtccagacccagcttcctgagcactcagccctccctctgggtacacagcagccctggtgggcagaattccttgtgtttacctcgttgtctgggtgatggtggtgattgggtggaactcccatgccagatatactgccaaggccaccctgaggggctgccaaccggggccccatgagtgctagagctgctggtcttgtgacgtgggcacctgtctgccaggcctgggcacgggcccagggtcacgctcatgctggggctcccgtcagctgactattttgggctcttgctgacctgggccaaagtcaggtccagtcaagagggcccaaagtggctccccccatccccagaggccggctctggtttctgctatataaacagaggaggacacgccggcttggaggcagcaccacatccagagctcgctcagctgctgcccagcctcggctgtgaggataggctggctgggcagcacgtctctccccacagggctccctgagaccaccaggaagagcccccaaccaatcttgggattctcccttcgtgcggttgtctgggacctttttccagggtcaaagcagatcgtgaggaggaagctggtgagtcttgggacagggacattgaggaatgggagctgggctgtttggattttttcccattgtgactgtcatgccaaaccctttggccttgggctgcggggaggaagcaagggtggccgacaactggaggcatgagtgtgcagcttgaccacagctggatgacggtagttggacttgtgaacgctgtgactgagaactaggagaggtgtacagagacagctaatgcagtctagctcctttctcagagcttccccacagggtgtccgtcctcctctcctctccaccccgcctggatgaggcactggtcactgcggcgagtttggtgtccattggggtatctcaggattggcttctgagtggggcccctgagcccccactagttgggactgagtggctgggccaggggcttggcgttctgctgagccctcagtccctggcctgccccgcttgccgggcccttgcccggcctcccagctgctttgggtgcctgagaagcagagctggagagaggtctggcaagtccgccaaccagcttgccacaaacacgctccgggacctgggccagggactctggcccggcatggaacaggcagcagctccttacgtgggaggctcctgtgctctccttcccagttctcaaaaaaaaattttcccctttctgaacgtgagtctctaaacgaatccagagcctgtgactaaaaatactttaacaataggaaagtgcatcagtctttccggctgggactcgttatcagagagtttttattttggaatttgaaattttgcccacgtaggctgggtggagaccttgggcagaggcaagggaatttggtgggtgctgatgttctgcaaggtggtgggagaccctccaaggcgagaggagaagagagctggccaggagctgaagagactgagagggaaactgagggaggttcaaggcgtggggcaacctttcccaaaggaactccgtgatcttgtctgtgttttccagccccgtctggtggttcttcctgaagtctgatcttattcttttatgctgtatgttaatttcttctggcctgagagggagggagggaggggtagagaaatcactggactgaagatagcctctgtggccatagcctgacctccttaattaactagctctctccagagtattaattgaccactcgctgcatacacagtctagaattctcccagtgctgggtagagccacatctctgggcacactctcttttcctctcactcagcccaggagtgggagaagtagagatggggttactgtttgtctgtagttcctggagcaggagggctgccggctcccccgtccagctgaggaccctgctatctcctgcccctgccaccctccctgccagccccaggattctgctgcaggctgctgtggttgtcccgttgcagacatgagttccacagggccgagtgtgggacttgagtatgcatggattttggtatatgtcggggtcctggaacccatccccctgtagcacttccctgtgccctggaggtactgcctgcctcccgacttccaccctcacttgccattacaggtttcccatgacagtgggggagcccaggccccaggcagtgctacatttggatgtggagcctctcctttccattgtgaactgaattctaccagcactgggcctggtgatgtctgcattaggcagatgaggaagcagtcttggagaggacaagggactggcctcaggtcacacatcaggcttgctagctcctggacaaagatgcctctgatagctcttccatatttcgcccctgctgagagctgaggggaggagtgcggccactagctgacctgcctgggtccctgggaggctggttttggggaggtagtgatacatgtgctcatggtagggcttgggcaagggccaggagagcagagcctgggcctgacctttcacccctggttcccagagctgcccacgggaagcccccagggcagtaggcagtgcttccctgcccacagtgcaaatccccctgcctactgccaaagcaggaagaggaagtggccgggtcagggctggcaataggccctggggccgaggaggggaagctcccagggccacttctgagctggagaaggcaaagctcactccttctgcccaccccaggggaagggtgtggaggcccatgggtggggacagtctcagtgaaagtagggcaagttcctgacctctactcagtaggggtggggcccagcccaaggtggaggggcagcgtgggaaaagggctgtgtaattccacttccttcatcccctgtttacccatctccacccctaccctctgggctggggaatggtggtggacattggctgctcacttggggcctgccctgggctacttactactgaggatgcacaaaggatcttcccaccacggactaggagacagtgcccttgtcctcatgttgttcacatactggtcggtgagggatgggtctgagtacatgataaatccctgggccccataagatttgactaggaggggtaagggcctggtgaactgctcagaaaataaactgtgttgtcagcaggaggacccctttctactatgagatattgcattccctgctaatcatacttgtgctctatctgttgatagaacaaatacataatgagaaacagccactgtgctttcagaagaacacacttgaaagaatttgttctcaataaattacatctgcatctggatccatatgaagaaatacgttcctctcattcaggcgtggctattttaaaataaaatttgggccgggcacggtgactcacgcctgtaatcccagcactttgggagtccgaggcaggcggatcacgaggtcaggagatcgagaccatcctggctaacacagtgaaaccccatctctactaaaaatacaaaaaattagccgggcgtggtggcgggcgcttgtagtcccagctactcaggaggctactaggaggaagggaaacagtatgcacagtgggtgaaacagtatgtgcaaagtgggtggtgggaaggtttagctcaaggtccttatatggggtcggtggggaactgcggtgatgagggcagatggataggctgggccgtggtgtgggggcctgactaggtggggctgtctcgtctccatcgtgacatctctccccgcccttccatgttgtcatggccttgccttacctccaggtccctgtctctagttttggatacctctctcattattacctgttccctcctaccccatctccccgacaattagctgttcactctgaccttgttcttgaaggttagacttggaggggtgtgtgctgggagtctgtggccagtctgggaccagcatttgttttaggcctcccaaagtccacgtcaattgtgcctttctccaaagttccactagctggaccccactctctcagaggcccaggccattctcccttgagggcccacaaggcctgggatccccaagcccctttcacctctctcgcctttatcttaggctaggtgctccacacgggaacctgcattggtggccccagctgtgtttccacagagggactttgcctttttctccttatttacttattttcagaatgtggtgtgttgagcacccccaccatctcccacccctgcaagcatgaccaggtggcagctgtagttccttatcctcctccaggctttggggcagcaggactgcagcctccataacctgcagcagcttggggaggtggggcctgggctgcaggcccagatctggaggctagatgctgagattcagaccagggacctggacagctcctcaccacactcagttttccttgggcttatgggtgggcctaccccacaggatggtctgtgccactttttacaaatgccaagttgtgaggaggagaggagggtgcctgagcttgaggctggttcaactctcaaagagctagcaaggctgggccgagggcggtggctcacgcctgtaatcccagcactttgggaggccaaggcgggtggatcacgaggtcaggagatcgagactatcctggttaacacggtgaaaccccgtctctactaaaaatacaaaaaattagctgggtgtggtggcaggcacctgtcgtcccagctactcaggaggctgaggcaggagaatggcatgaacctgggaggcggagcttgcagtgagccgagatcgcaccacttcactctagcctgggcgacagcaccatctcaaaaacaaaacaaaacaaaaacaaaaaacaaaaaaagagctagcaaggctgagattgagcacagactggtcaagccacagataacgcacttgttcttgctggttggcaggaagaacaatggcttggctgatcatcttttgctacctattaaaattggttttattttggccgggtacagtggctcatgcctgtaatcccagcactttgggaggccaaggcgggcagatcacaaggtcaggagatcaagaccatcctggctaacacagtgaaaccccgtctctgctaaaaatacaaaaaaattagctggcgtggtggcaggcacctgtagtcccagtgactcgggaggctgaggcaggagaatggcgtgaacccgggaggcggagcttgcagtgagccgagatagtgccactgcactccagcctgggtgacagagcgagactccgtcttaaaaaaaaaagttagttttattttctagggaaggccctgctggggtggagggagacatcctggggcaaagtgggtgctaggtgaggaagtcagcctgtgacacagctgggatcagttccacccagcaagacttgctgtgtgctcctgagcaaagtgtagctcctcttggaacccttgactcctaatctgcaaaatgggaggacttgaattctatatgtggttttaaaccttttttttttttaagctgtggagtcattttccacaaaaggattccacaaaatctaagcaaaatcttacggaaaagcctaagagaaagaaaagcaaaactttgcctgaaatgaggtagggatcctggatgggtccaattggcatcatcctgcccacacttcacattcaaccagtggccttgaaggaagatggtttggagcaacccagagctctctgcagatgcattcacgttgagactgtatgctgggatggagggttctggcaggttctgaccagatgactgaatgccatcagcattacagtcaccccttggtttatgaagggggttggctccaggaccccaacttgtactgcgaatactcaagtcccactgttggccctgtggaacctgtagatactaaaagtcggccctctgtatatgtgggtttttggatccatgttggattgaaaaaaaatccccaaatccccatatgagtggacctacacagttcaaacgtgttgttcaagggtcaactgtgtatgaagactgctccatctgtggtggacagtgaggctttagaaagaggagacaggcaggctaatggatggtcttcatgggtgtgggaaaggaggagtggtcactgggcgcaggcagttggtctgtgggaccagtgaggcatgggctgtggctggcagtaggaaagcgggagaggctgagtgcagtggctcatgcctataatcccagcactttgggaagccaaggcaagaggcttgagcctgggaggtccaggctgcagtaagccataatcatgccactgcattctaggctgggcaacagaacaagaccttgtctcaaagacaacaaaacaaaacaaaaacggggagaggaaaggacccaagagatgttccaaaaaagaatttcaggaaacagtgaaaagttgcaaatggggcatcaggagccaggagcagagattaggtggcaggcagggggtgcagaaatgacaagttctgtctggaacagatgaaatttgaggtgcttgtgggacctttgagtgggcagaagcgggggctggagacaagggtgtgggaaaactgtgatgggggttgagggtggaaatcgggccggcagagcctccggggcagagaaagtcctatggggtgaccagatgagttgagaactgggtgggtgggtgcaggctcaatggaagcagaaactcatggatattgacttacatgggagaaggggaatggatgggggtcgcagtggggtggcagggctctcttttccttgtttttgtttttttgcccttccctcttctcttacttttccgtgagggctccccaggctcaggagagatcagggtggaagggtgacggccaaaccagggaaggctccaggtggctgaagcctggtctgtgcccagcagggccctggcgggctgttcctcactccaccgggcaggtgataactggtcagagctgcctccccacaggtgctggaggtaggctgggagggccggtgctccctgatgtggacagggggaggggtattgataagaggcgtggagagatccctagagatgcagtctgtggccctgggttccaacccagtgtgccaccacctggctgtgtgaccttcagcaagtgccattatttctctgagcctgtttgtttataaaatgaggaagagttggcacaagtttgaaaagatttctcaggctccacccggttctgaaattcggtaatttcccaactagggtgcactccccctgtaaggggctggggaggggacggtgcgaaaccaagttcagcttgtggagcggagccagagctgttggccgagcttgggcctggccaacgcctgccctcgggaaggtcctgtgtagggagactgcctggagggactaagcgagggctctaactgacgtctcaggggcagcctctcagcctgagaccctgctggggaagccgcgtcctgtgcactagctgcgcctagggctgaggtgagggcgcaggctccccagggtgtgtccgaattgcccgcctcagcccgcggcctgtcctgaccgcccagcagcggcagcagcgacaccctagggctccaggaagggcttgggaaggtgtaaaggcggggctaggctcggagggagccggagggaccgggcgcggttggctcccgggagcaactggagagtgaggagatcctcatccggggaagccccgcggccgcgtctctacagcgccccttctcgggctctggccctcccgctggttattctggacctgggggcccccagctgggacccgagtccggtgcggggagcctagtgggcctgggagctgctatttttagcgggcgcggcgggcgcgaggagcctatttatagatcaaacaatccgcgctccctgcgtcaatggaaccccgcgtgcgtcacgcgcgcagacattccaggccccccctcctcgccccgccccctcgggctccccgggccgcacctccccctggccgcctcccgccggaaccgcaccgccccccgcgcccttgtatggccaaagctcgacgggcggcctgcgtcagtggcgcccccgcccctccccgtgcgtcacggagcgcttaagaggagggtcgggctcggccggggagtcccagtggcggaggctacgaaacttgggggagtgcacagaagaacttcgggagcgcacgcgggaccagggaccaggctgagactcggggcgccagtccgggcaggggcagcgggagccggccgggtaggttcccttcggggaacgtgcatctgtttttaggagcggtgcatgaaggagatgggtgtacgcgcgggcagagaggatgttgtagggccggcatgcaagagggtaggtgtagtgtgcagctgtagcacaacgggatgagaacccaggtcagactggaaactactgggtgcggggtaggaggtaggggaggttgactacctgcaaagtggaagctgtagggggttggggttggcgccgggagtagggacttgtccagcaggcggctggccttagttcagtctcctagggtgcccaactgggcgcacggtgctctgaacttgggtgggtgtggatgggggtcacagcaccgtgggggaacaggtacacagagctttggcgctgctgggatccgtacgattctaactgggattggatttccaaaaggccctccttcctcccagcctgtcagctccctcccagtgtcccggttgttttcttaacactttagaggctgtggctgagttggcaggggttccccaccctgaactttctgtctgaggtgcgttaggaagcctccggatcattgaaggaagtgactggcttggtggctgacctgtggagggctgcttgctgtgtattgaataaaggaggcttcttggggtcggggcagggtctctgtaagcacagggagaattgatttcgctggggcttggatcacgtgtgtgtgtgtgtgtgtgtgtgtgtgtgtgtgtgtgagagagagagagagagagagagaaagagagacagaggtggaaacccttgtccttggggatcctggatagaagttgccagtgttggagtcttggggcggtgctgcctagaggatacctcccaaccattccagctccagatgctgatcatccctatgccccctcccccagtagtctctgttggatcttacaggtagggtgcagcctgaggcttgttcagcagaacaggtgcaagccacattgttgccaagacctgcctgaagccggattctccccactgcctccttcaaccccgcctcttcctcctcctgtgggactgctcccccctcctgtgaggctagatgtaggtccatatcttgtgttgttaagaacctgcatgaagggggagggactgatgggggttgatccggatgtgggacgtccaggtggagaaacaggatttgaataaggcaggaacaagcgcccagttctgcccaactgctgtctttcctccttcacccccagcccatccctggccaggctgtcctagtgcaggcagatgtggccaccctcacctctcgcagggtgctgcatgcctgtggactggtgccactgggtgtgtggggcctgagctacaagggtctctgttgtcttggttgtcaggctaggggtggaggtgccctgttcagggcctgtggagttagaaggaaggctaggaccagagaggacttcctgggctcttggccaggaggtggtgacagcctttgcccccatcactgcaccccttgattatggtactctgttcctgcttgatttcttctccagcttccctcattgactggtgaggccagagttacgcagaccccaggtgtgtaggttgggagggagactgtcctggagcctcctctgcattattttttgaccattctgtgaatgtaaggatctgcctgaatgatgctatctctgttcaggctctcttcaggctcctcgggactggggctgtgtaaccccctttgcctgagcctctggattcttcagggcacagaccctgctttagtggagggacccaggcaggggcagaatgtcttccacccacccaccagcaggcttctccttaagtcttctgctggactctcagagttctcttattaagataactgggggcagcagaacattctttttaggtactgttgtccattctcctgagagtccagggggctgtggtctccttcctccccccttgccctgtccccagcaaatggcctttaaaggtctggagccaggttaccgatgaggaggcctaggttcccttcctctttgcttctgggagcatctcgagcagtgcagagtggcttcccagccccagatggcaggattggggtgggtttgggatctgctgcctttgctcagtgtgcagggttggggtggaaatgggggacaggctagggcctctgcattaggctgccttcttaggcaggtgggctttcacttccagctcctcttccattttcacgatgtccttcttcctaggcactgcgaggggaggagaaaaggggctttgcagaggcctgggagtattcccaggaagtgcctggttggcgaaacaaccagggagctgctcctgggagctgggctgaggctttgctgggctgcctctcccactcacccttctcccggcccccaccatgccttccccatgggggaggggcagggggctggaggaacacagcttccccctgttctagcagagaaatgctggctgtatgcctcccctagggttcccaggctgactagggtgtggctggccttctgatggagcccactcatgctgggccgctgcccaggggctttgtggcacctaggtcgagatggtactcaggccaggggtcaggattcctgggtgctctggtcccggtgcctctgtctcatctttaggctgggattcctgccaccttgctgctctggggcccaaatactttgagacaaggctataggcttgtcccactgactctcctttccctccctggggtctcctctctctccagagatgccctgtatccaagcccaatatgggacaccagcaccgagtccgggaccccgtgaccacctggcaagcgaccccctgacccctgagttcatcaagcccaccatggacctggccagccccgaggcagcccccgctgcccccactgccctgcccagcttcagcaccttcatggacggctacacaggagagtttgacaccttcctctaccagctgccaggaacagtccagccatgctcctcagcctcctcctcggcctcctccacatcctcgtcctcagccacctcccctgcctctgcctccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccaccggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtggaagtgaaggccgctgtgctgtgtgtggggacaacgcttcatgccagcattatggtgtccgcacatgtgagggctgcaagggcttcttcaaggtaccgcgcagccccaggtggggccttttgttggaaatggagagaggctggcctcatcccattgggacctgtggtctccccctgggttctcctcctagctaagtcctgtcctgcagggtgggatcagccctgccaggtgggccgccttcctggagacccgtagatgccagggctggaagctttcatttgccgggacactcgggcccatgggattgcacagagctggagggaggggtgagataggggcagataggagctgcaggggtgcctggcgagcctctggttttcctctgctcctctgcctgtcctctcccaactcaaggttctagtgggaaggggtgcccccaggctctcatgttcctggcgtgagatgaaaggatccctgcggagggtttggttcttgagggctgggggtggacttgggaacaggctgtgtgtttgtcccagcgatggtgcctgcttagcttcccgtccccaccccccagccccttggccctctcctgtctgccctagggagaaggcaggtggacaagggcccatgaaaaaatacaggtgtctagactgccagggagaccctggcccccagtagtgtgtcctggggacttcctcagagcgagaaacctcccccaatgtcttcaagacttttctctccccccgcccaaccccgtctctccctcccttgccacccaaatgttagaaaaatagctgtgaacagagagcgcttttgtctgcaatggcagcaggatctggacggtcccctcccctaagttcccccctccccaccccacactctgacagcttgttccgtgttgcccccccacccagcgcacagtgcagaaaaacgccaagtacatctgcctggctaacaaggactgccctgtggacaagaggcggcgaaaccgctgccagttctgccgcttccagaagtgcctggcggtgggcatggtgaaggaaggtgtgtggctggggtgcggcccagcggggcaagggtaggcttgagtggagtgggaccagcagggcccccaggcttctgccctggaggacccagaggagggcacgtcttatttccaccccacctctgaaccccaggccttggagggaggcagcctacacctgcctggattgtgagggtggtggcagggggaggttcctatagggtaccttggatctcagggactctgggtcctagggactcggtggggcgcgtctcagcagtggtgtgcacggcttgggctgagaggcccttcctcagatcccttccttcctcacccctacccattcctttgcagttgtccgaacagacagcctgaaggggcggcggggccggctaccttcaaaacccaagcagcccccagatgcctcccctgccaatctcctcacttccctggtccgtgcacacctggactcagggcccagcactgccaaactggactactccaaggtgaggtcccaccccgtgtctgccttggggaggtctatgagcacatgcagtgcctttgtgcgtgttaggagagctaccccctctggaaggactgaatgagaaaggaggtttaaaaaagaaagaaagaaaagcgactccctccagttcgacagatcaaagagaggatccccctctcggctgaccagatgggaaaatgcaccccctcaggcaggtggccaattagaaaaatatgtccttttggcagctgcagccctgggttaatatgtgagacttggcaagtgagagcctgggcaggatctcagatccactcccactcccgggatctggcatccaagtgtctgacacagccatacgtggcagtgggtgtaggagcctgcctggggtgctgaccccactggaccgtcttcctagttccaggagctggtgctgccccactttgggaaggaagatgctggggatgtacagcagttctacgacctgctctccggttctctggaggtcatccgcaagtgggcggagaagatccctggctttgctgagctgtcaccggctgaccaggacctgttgctggagtcggccttcctggagctcttcatcctccgcctggcgtacaggtgagagccactgactgtctgcccagcccctttccctgatacacctgcctgtgaaccaccctgatcgctcttcgtgcccatctgcctgccaggtctaagccaggcgagggcaagctcatcttctgctcaggcctggtgctacaccggctgcagtgtgcccgtggcttcggggactggattgacagtatcctggccttctcaaggtccctgcacagcttgcttgtcgatgtccctgccttcgcctgcctctctgcccttgtcctcatcaccggtgagtgaccagcaccacaccaggtccaagggaatgggcgtcagggggttgactggttctcaggagggcactgtcccagggagtttggtgggccgggatctagcactttctgggcccctgcactgccccgggctcatgccagcagtaaagtagggaccataggaattgcagatgggctcagctgcatccagctctaaagcgcaagacagcctttggtcccagactgtcaggatccagactgcaggtgtcatgtatgtggcctagggtattggcttgtttttaaaaccttcctgtcaattatgctgcatatagaaaagtacacaaatggtaaattttcataaaccgcacttacccaaccagcgccttggtcaagaaagagcacataactgccccccagaacccattattgtgcccccttccagtcatttgccctccccaagggtaaccactactttttatttaaatattttaaaaataagatgggaatctcactatgttgcccgagctggtctcgaactcctgggctcaaatgatcctcctgtctcagcctcctaaagtgctgggattacaggtgtgagccaccacgcctagcccttcactgtgacttctgacagtgcagatcagattggttgtgcctgttttggactttatgtaaatgtagttctgcaggatggaatctggtgttgaatgcagaggttttcagatttctctgttttttaaaggaaagaatccaccctcgttcattttttcacttaaattgcacaggggacccaacgatatagaacacaatcagaggtactctgggctgagggagtgctgagttctgaggctgggtttctcagaacagtctagattttaaaaacccaatgatctagccagaaaacgtaggttaggattttatttcccgtttgtgaccctgggcaagtcattagcctcctgggcctcgggttctcacttggagtatgaggataatgagggttactgcttctcagacttgtgacgatgcttactaatggccaacatgtgaatgcgcttttgtgaagtgccagcagagcatgaggggtggtcaggggcagcagttttaggggcctgggggaggctggggctttgggggcctggttctcagatgtacagctaatcctgtacccttcccgcagaccggcatgggctgcaggagccgcggcgggtggaggagctgcagaaccgcatcgccagctgcctgaaggagcacgtggcagctgtggcgggcgagccccagccagccagctgcctgtcacgtctgttgggcaaactgcccgagctgcggaccctgtgcacccagggcctgcagcgcatcttctacctcaagctggaggacttggtgccccctccacccatcattgacaagatcttcatggacacgctgcccttctgacccctgcctgggaacacgtgtgcacatgcgcactctcatatgccaccccatgtgcctttagtccacggacccccagagcacccccaagcctgggcttgagctgcagaatgactccaccttctcacctgctccaggaggtttgcagggagctcaagcccttggggagggggatgccttcatgggggtgaccccacgatttgtcttatcccccccagcctggccccggcctttatgttttttgtaagataaaccgtttttaacacatagcgccgtgctgtaaataagcccagtgctgctgtaaatacaggaagaaagagcttgaggtgggagcggggctgggaggaagggatgggccccgccttcctgggcagcctttccagcctcctgctggctctctcttcctaccctccttccacatgtacataaactgtcactctaggaagaagacaaatgacagattctgacatttatatttgtgtattttcctggatttatagtatgtgacttttctgattaatatatttaatatattgaataaaaaatagacatgtagttggaactga

SEQ ID NO: 2 - RNA, 2635 nt, Homo sapiens
  source 1..2635: mol_type = mRNA
agtcccagtggcggaggctacgaaacttgggggagtgcacagaagaacttcgggagcgcacgcgggaccagggaccaggctgagactcggggcgccagtccgggcaggggcagcgggagccggccgggtagggtgcagcctgaggcttgttcagcagaacaggtgcaagccacattgttgccaagacctgcctgaagccggattctccccactgcctccttcaaccccgcctcttcctcctcctgtgggactgctcccccctcctgtgaggctagatagatgccctgtatccaagcccaatatgggacaccagcaccgagtccgggaccccgtgaccacctggcaagcgaccccctgacccctgagttcatcaagcccaccatggacctggccagccccgaggcagcccccgctgcccccactgccctgcccagcttcagcaccttcatggacggctacacaggagagtttgacaccttcctctaccagctgccaggaacagtccagccatgctcctcagcctcctcctcggcctcctccacatcctcgtcctcagccacctcccctgcctctgcctccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccaccggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtggaagtgaaggccgctgtgctgtgtgtggggacaacgcttcatgccagcattatggtgtccgcacatgtgagggctgcaagggcttcttcaagcgcacagtgcagaaaaacgccaagtacatctgcctggctaacaaggactgccctgtggacaagaggcggcgaaaccgctgccagttctgccgcttccagaagtgcctggcggtgggcatggtgaaggaagttgtccgaacagacagcctgaaggggcggcggggccggctaccttcaaaacccaagcagcccccagatgcctcccctgccaatctcctcacttccctggtccgtgcacacctggactcagggcccagcactgccaaactggactactccaagttccaggagctggtgctgccccactttgggaaggaagatgctggggatgtacagcagttctacgacctgctctccggttctctggaggtcatccgcaagtgggcggagaagatccctggctttgctgagctgtcaccggctgaccaggacctgttgctggagtcggccttcctggagctcttcatcctccgcctggcgtacaggtctaagccaggcgagggcaagctcatcttctgctcaggcctggtgctacaccggctgcagtgtgcccgtggcttcggggactggattgacagtatcctggccttctcaaggtccctgcacagcttgcttgtcgatgtccctgccttcgcctgcctctctgcccttgtcctcatcaccgaccggcatgggctgcaggagccgcggcgggtggaggagctgcagaaccgcatcgccagctgcctgaaggagcacgtggcagctgtggcgggcgagccccagccagccagctgcctgtcacgtctgttgggcaaactgcccgagctgcggaccctgtgcacccagggcctgcagcgcatcttctacctcaagctggaggacttggtgccccctccacccatcattgacaagatcttcatggacacgctgcccttctgacccctgcctgggaacacgtgtgcacatgcgcactctcatatgccaccccatgtgcctttagtccacggacccccagagcacccccaagcctgggcttgagctgcagaatgactccaccttctcacctgctccaggaggtttgcagggagctcaagcccttggggagggggatgccttcatgggggtgaccccacgatttgtcttatcccccccagcctggccccggcctttatgttttttgtaagataaaccgtttttaacacatagcgccgtgctgtaaataagcccagtgctgctgtaaatacaggaagaaagagcttgaggtgggagcggggctgggaggaagggatgggccccgccttcctgggcagcctttccagcctcctgctggctctctcttcctaccctccttccacatgtacataaactgtcactctaggaagaagacaaatgacagattctgacatttatatttgtgtattttcctggatttatagtatgtgacttttctgattaatatatttaatatattgaataaaaaatagacatgtagttggaa

SEQ ID NO: 3 - RNA, 2485 nt, Homo sapiens
  source 1..2485: mol_type = mRNA
agtcccagtggcggaggctacgaaacttgggggagtgcacagaagaacttcgggagcgcacgcgggaccagggaccaggctgagactcggggcgccagtccgggcaggggcagcgggagccggccggagatgccctgtatccaagcccaatatgggacaccagcaccgagtccgggaccccgtgaccacctggcaagcgaccccctgacccctgagttcatcaagcccaccatggacctggccagccccgaggcagcccccgctgcccccactgccctgcccagcttcagcaccttcatggacggctacacaggagagtttgacaccttcctctaccagctgccaggaacagtccagccatgctcctcagcctcctcctcggcctcctccacatcctcgtcctcagccacctcccctgcctctgcctccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccaccggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtggaagtgaaggccgctgtgctgtgtgtggggacaacgcttcatgccagcattatggtgtccgcacatgtgagggctgcaagggcttcttcaagcgcacagtgcagaaaaacgccaagtacatctgcctggctaacaaggactgccctgtggacaagaggcggcgaaaccgctgccagttctgccgcttccagaagtgcctggcggtgggcatggtgaaggaagttgtccgaacagacagcctgaaggggcggcggggccggctaccttcaaaacccaagcagcccccagatgcctcccctgccaatctcctcacttccctggtccgtgcacacctggactcagggcccagcactgccaaactggactactccaagttccaggagctggtgctgccccactttgggaaggaagatgctggggatgtacagcagttctacgacctgctctccggttctctggaggtcatccgcaagtgggcggagaagatccctggctttgctgagctgtcaccggctgaccaggacctgttgctggagtcggccttcctggagctcttcatcctccgcctggcgtacaggtctaagccaggcgagggcaagctcatcttctgctcaggcctggtgctacaccggctgcagtgtgcccgtggcttcggggactggattgacagtatcctggccttctcaaggtccctgcacagcttgcttgtcgatgtccctgccttcgcctgcctctctgcccttgtcctcatcaccgaccggcatgggctgcaggagccgcggcgggtggaggagctgcagaaccgcatcgccagctgcctgaaggagcacgtggcagctgtggcgggcgagccccagccagccagctgcctgtcacgtctgttgggcaaactgcccgagctgcggaccctgtgcacccagggcctgcagcgcatcttctacctcaagctggaggacttggtgccccctccacccatcattgacaagatcttcatggacacgctgcccttctgacccctgcctgggaacacgtgtgcacatgcgcactctcatatgccaccccatgtgcctttagtccacggacccccagagcacccccaagcctgggcttgagctgcagaatgactccaccttctcacctgctccaggaggtttgcagggagctcaagcccttggggagggggatgccttcatgggggtgaccccacgatttgtcttatcccccccagcctggccccggcctttatgttttttgtaagataaaccgtttttaacacatagcgccgtgctgtaaataagcccagtgctgctgtaaatacaggaagaaagagcttgaggtgggagcggggctgggaggaagggatgggccccgccttcctgggcagcctttccagcctcctgctggctctctcttcctaccctccttccacatgtacataaactgtcactctaggaagaagacaaatgacagattctgacatttatatttgtgtattttcctggatttatagtatgtgacttttctgattaatatatttaatatattgaataaaaaatagacatgtagttggaa

SEQ ID NO: 4 - RNA, 2586 nt, Homo sapiens
  source 1..2586: mol_type = mRNA
ttcctggtgtaagctttggtatggatggtggccgtctccctacagactgggagctgttagagggcagggatcctagctgacacatctatgtcctcgccttggttggaggcctccaccatggacagaggccaggccctgcccctcccaggcagcctggctccttctgctgggccctgaaggcagacgggataatgtggttggccaaggcctgttggtccatccagagtgagatgccctgtatccaagcccaatatgggacaccagcaccgagtccgggaccccgtgaccacctggcaagcgaccccctgacccctgagttcatcaagcccaccatggacctggccagccccgaggcagcccccgctgcccccactgccctgcccagcttcagcaccttcatggacggctacacaggagagtttgacaccttcctctaccagctgccaggaacagtccagccatgctcctcagcctcctcctcggcctcctccacatcctcgtcctcagccacctcccctgcctctgcctccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccaccggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtggaagtgaaggccgctgtgctgtgtgtggggacaacgcttcatgccagcattatggtgtccgcacatgtgagggctgcaagggcttcttcaagcgcacagtgcagaaaaacgccaagtacatctgcctggctaacaaggactgccctgtggacaagaggcggcgaaaccgctgccagttctgccgcttccagaagtgcctggcggtgggcatggtgaaggaagttgtccgaacagacagcctgaaggggcggcggggccggctaccttcaaaacccaagcagcccccagatgcctcccctgccaatctcctcacttccctggtccgtgcacacctggactcagggcccagcactgccaaactggactactccaagttccaggagctggtgctgccccactttgggaaggaagatgctggggatgtacagcagttctacgacctgctctccggttctctggaggtcatccgcaagtgggcggagaagatccctggctttgctgagctgtcaccggctgaccaggacctgttgctggagtcggccttcctggagctcttcatcctccgcctggcgtacaggtctaagccaggcgagggcaagctcatcttctgctcaggcctggtgctacaccggctgcagtgtgcccgtggcttcggggactggattgacagtatcctggccttctcaaggtccctgcacagcttgcttgtcgatgtccctgccttcgcctgcctctctgcccttgtcctcatcaccgaccggcatgggctgcaggagccgcggcgggtggaggagctgcagaaccgcatcgccagctgcctgaaggagcacgtggcagctgtggcgggcgagccccagccagccagctgcctgtcacgtctgttgggcaaactgcccgagctgcggaccctgtgcacccagggcctgcagcgcatcttctacctcaagctggaggacttggtgccccctccacccatcattgacaagatcttcatggacacgctgcccttctgacccctgcctgggaacacgtgtgcacatgcgcactctcatatgccaccccatgtgcctttagtccacggacccccagagcacccccaagcctgggcttgagctgcagaatgactccaccttctcacctgctccaggaggtttgcagggagctcaagcccttggggagggggatgccttcatgggggtgaccccacgatttgtcttatcccccccagcctggccccggcctttatgttttttgtaagataaaccgtttttaacacatagcgccgtgctgtaaataagcccagtgctgctgtaaatacaggaagaaagagcttgaggtgggagcggggctgggaggaagggatgggccccgccttcctgggcagcctttccagcctcctgctggctctctcttcctaccctccttccacatgtacataaactgtcactctaggaagaagacaaatgacagattctgacatttatatttgtgtattttcctggatttatagtatgtgacttttctgattaatatatttaatatattgaataaaaaatagacatgtagttggaa

SEQ ID NO: 5 - RNA, 3513 nt, Homo sapiens
  source 1..3513: mol_type = mRNA
tttctccgccggctgcgggaaccggttccaggagcgcgggagctcgtctgccaggagacccagccgggagggaccgatgctgcgtgatgcaaccccttcgagggactgagtcaagaaaccaaggctgggggtctcgtggagtcaggcggagctggggattaggactcggggcttgtgtctcccaggcgtgtttttttgttgttgttttgtttttttgccgcaacatcttccttatactgggtggggtgggggcgggggtggtctgtatttccagacctcctccatggtggtctggctgtgtggagggcagggcacagttccttgacttggctcattcccccccaacgccccccctttcttttttttttgagatggagtctcgctctgttgcccaggctggagtgcagtagcgtgatctcggctcactgcaatctccgtctcccgggttcaagcgaatctcctgcctcagcctccagagtagctgggactacaggcacccaccaccacgcccggctaatttttgtatttttagtagagacagggtttcaccatattggccaggctggtctggaactcctgaccttgtgatccgcctgcctcggcctcctaaagtgctgggattacaggcgtgagccaccgcgcctggcaggctcattccctttttatgccttcgtttttgatagatgacctggggatgggagaaatgctgcagaggtggtttatttacagtgaattgttaatttggttgtggggctagtattaggggcacattgggaaaacacaggctgggcagaggttggtaggttcacaacagttatgcccactgggtgctgatggatttgggggctgtcgtcggcctggcttggggccctagtggtggggctacaaggcttgctcctagatctactccaagtgaacatttaagatcagtgacttggatggagacttaaaaaggaagtcagctgactccgtgtagtcatccagagctgtgaggaatgacaagtgcacagtataaaatcaagattctaattgaaggcctccaccatggacagaggccaggccctgcccctcccaggcagcctggctccttctgctgggccctgaaggcagacgggataatgtggttggccaaggcctgttggtccatccagagtgagatgccctgtatccaagcccaatatgggacaccagcaccgagtccgggaccccgtgaccacctggcaagcgaccccctgacccctgagttcatcaagcccaccatggacctggccagccccgaggcagcccccgctgcccccactgccctgcccagcttcagcaccttcatggacggctacacaggagagtttgacaccttcctctaccagctgccaggaacagtccagccatgctcctcagcctcctcctcggcctcctccacatcctcgtcctcagccacctcccctgcctctgcctccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccaccggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtggaagtgaaggccgctgtgctgtgtgtggggacaacgcttcatgccagcattatggtgtccgcacatgtgagggctgcaagggcttcttcaagcgcacagtgcagaaaaacgccaagtacatctgcctggctaacaaggactgccctgtggacaagaggcggcgaaaccgctgccagttctgccgcttccagaagtgcctggcggtgggcatggtgaaggaagttgtccgaacagacagcctgaaggggcggcggggccggctaccttcaaaacccaagcagcccccagatgcctcccctgccaatctcctcacttccctggtccgtgcacacctggactcagggcccagcactgccaaactggactactccaagttccaggagctggtgctgccccactttgggaaggaagatgctggggatgtacagcagttctacgacctgctctccggttctctggaggtcatccgcaagtgggcggagaagatccctggctttgctgagctgtcaccggctgaccaggacctgttgctggagtcggccttcctggagctcttcatcctccgcctggcgtacaggtctaagccaggcgagggcaagctcatcttctgctcaggcctggtgctacaccggctgcagtgtgcccgtggcttcggggactggattgacagtatcctggccttctcaaggtccctgcacagcttgcttgtcgatgtccctgccttcgcctgcctctctgcccttgtcctcatcaccgaccggcatgggctgcaggagccgcggcgggtggaggagctgcagaaccgcatcgccagctgcctgaaggagcacgtggcagctgtggcgggcgagccccagccagccagctgcctgtcacgtctgttgggcaaactgcccgagctgcggaccctgtgcacccagggcctgcagcgcatcttctacctcaagctggaggacttggtgccccctccacccatcattgacaagatcttcatggacacgctgcccttctgacccctgcctgggaacacgtgtgcacatgcgcactctcatatgccaccccatgtgcctttagtccacggacccccagagcacccccaagcctgggcttgagctgcagaatgactccaccttctcacctgctccaggaggtttgcagggagctcaagcccttggggagggggatgccttcatgggggtgaccccacgatttgtcttatcccccccagcctggccccggcctttatgttttttgtaagataaaccgtttttaacacatagcgccgtgctgtaaataagcccagtgctgctgtaaatacaggaagaaagagcttgaggtgggagcggggctgggaggaagggatgggccccgccttcctgggcagcctttccagcctcctgctggctctctcttcctaccctccttccacatgtacataaactgtcactctaggaagaagacaaatgacagattctgacatttatatttgtgtattttcctggatttatagtatgtgacttttctgattaatatatttaatatattgaataaaaaatagacatgtagttggaa

SEQ ID NO: 6 - DNA, 8293 bp, Homo sapiens
  source 1..8293: mol_type = genomic DNA
cgcaagccacataaacaaaggcacattggcggccagggccagtccgcccggcggctcgcgcacggctccgcggtcccttttgcctgtccagccggccgcctgtccctgctccctccctccgtgaggtgtccgggttcccttcgcccagctctcccacccctacccgaccccggcgcccgggctcccagagggaactgcacttcggcagagttgaatgaatgaagacagacgcggagaactcctaagtgagtagatgcagcccatgcagttcgccttcttttatgctttttccttcttttgcacgtctcttctttccacttgtgtgggacaggttctctggaagtgggagccagaggcttctagtgagagtgggaccgaaggatggggagtgcgtgcgcgcagttaccggggggcatttgttcgaactccggctttggcactagtggggagttggctctcgacagaggtttccaggctcctcattggtggacgtggaagggagactccacagtttgggagctgaggactagcccgcggaaatgtgcgcaaagtttgctgttagtgaggaattgattgtggcctgtgaacacggagactccaagtcctatgtatacgagggaagctgcccacaaactgacagggagaggaagttcttcagtttatgcgttgcttgggaactgtgtctccgcggctggccagcgcgcgatgtttcccgggtattgttgagtaagggtggtgttggtagcgtgtcctgttactaagttgcctgaaatttctggttttgacatatgctgtccttgggtttgcgaatgtatcagagcgagaatatgaatatgtaaagagtacagttatgaaactgtggagctaccagggggttaatatccaacacaggaatatctctaagggctgtggggttcgagtctcctttctgctttttctgggtaatcttccccccacccccaccaccactatgaagcaagattgtgggggaggggaaagaataaaaagagaggatactggctttcttttttcattagtaataagattgtctgtgctctagaatgtcttccaagtgggaacatctgaaaattgctaggacacaagaatgccttgttccagaaaggcagattgtggaaggcattatggggaaggtgttcatcttgctgtgctgggaaacacttctaatattggtgccaataccatataagcagtatgtcccccctctgcaattgacctaagaagctcctggaaaagtagatcccctcttcccaccttgtgaccattaagccttgtgaccattaaagatgctgaaagacaagttttctggaaaagtgaacatcaatttatctgtagctccaatcccagtgctctgtcaaaagcactttagaagtgcggatgcttccactcaacttgccttctcagtcaaggcctttctaacattttgtaagggggaagattgttttctcattttatattcttgacttctactttcttcccctctaccaaaagaaaaggcaatttcaccacaagaaaaaaaaatgcaagagaaggttccaatgctgtattttcatactctagtcttcatactcaggtcctgaattaacctaagatggaaatgacctctccacctacactgtagcaaaggggccagttcattacatcataaatgttaaatgagttcatggactagctttcctcttgcaggatcttctctctgcaaggatttacacagtgcaatgggtggtattttctgttgtttcaagtcatttcttttatacattcattttaagtgctatgtttggtaaaggcttcccactcatttccaatgagacaaacagggaaggcatggaagggcctgcctggtgagtctacatatgcccagctgaatctctgtcgggaagaaaccctgaagcttcctgtgtctgtatttcagggaggagattggacaggctggactccccattgcttttctaaaaatcttggaaactttgtccttcattgaattacgacactgtccacctttaatttcctcgaaaacgcctgtaactcggctgaaggttagtgcaacttcatttctttcctttactctccagagctccccaaacatcaagaaacaggacaaggcaaaccctgtaacttaaggtttgcccgacccatcgccttcgggaacaactttctcattgtgaaattcaacttcatttctagatggtcatttctagaaagagactgctgaatctgagcttcagagaagaggctcatctgagtgggatgagtgggggggtatgagggagatgtttggaaatacccaggagtgtagaccctcagtagctttttagctctgggtctttatttggttagtctttccacgccctaaactgttgttctgcagcattctctctctcctgcctttcctctcgcgcccctacatgctctctgactgccgcgggctgccggtgtagctccaggtgtacccgagcccgggagaaagtgttcagttgaccaggctgagtgtgttatcaccctgtttcatttccagccatgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgccagtctcccgcctctccctcttctcctttaagcaatcgccccctggcaccccggtgtctagttgccagatgcgcttcgacgggcccctgcacgtccccatgaacccggagcccgccggcagccaccacgtggtggacgggcagaccttcgctgtgcccaaccccattcgcaagcccgcgtccatgggcttcccgggcctgcagatcggccacgcgtctcagctgctcgacacgcaggtgccctcaccgccgtcgcggggctccccctccaacgaggggctgtgcgctgtgtgtggggacaacgcggcctgccaacactacggcgtgcgcacctgtgagggctgcaaaggcttctttaaggtgagcaatggcgggagcggagtaggcaggtagggagcccctagtgcccgggacctcggagtgtgccctctgccttggtgccagtagcccagccccagctctcccgggactgcccagctctccggggtccgccgaagctgccctgcaggagaccatgggctgcggcggggacttccgggtgtctgagaaagggaagcagaaagactgggaggccagggtcgcatccccctcgcattcagccgacccggctggcccccgcccgaagttgctggagccggagttggaagagggtcatttgcatgtgctaggagctgtcttccctgttcagaatgaaattggttaggacagagaaccgtgtctgagctaaccaagtggaacagaattccctatggtcaaattaagtgatctctttatttcgccatcctgattgaataatcttatcattttaaatagagaaggtctccaaggaatgtaaataatatgaatgcccacggatttgtatttactgagcgtctccttctccttctcttggcatataaaacacagcaaggagcggcaaggttagctcaaatgttaacgctatcaattttcttctgttaaatgccctgggggaggaaaaaagaaaagaaagaaagaaaaggaagagaaaaaaataaaatggaattgtgtgtatgtgtttgtttgtggggaggaatcgtagaccccagtcacataacagaaattttctccgagttgcctgattttcaaaagaagaaaaaaaatgttggtctatattgtctccttttgcagcgcacagtgcaaaaaaatgcaaaatacgtgtgtttagcaaataaaaactgcccagtggacaagcgtcgccggaatcgctgtcagtactgccgatttcagaagtgcctggctgttgggatggtcaaagaaggtaggctgaggggagctgccgaccctccagtttgcgcctttaggaaaccactgctcatactccagcatcacgttccacttcccggtgctggggatctccgactccccctcagtatggcctccaggaccctgcagctgcctgcttgcccggccttccctagagaaagccgccaggcccttctctcctttaactatacgacccatttggaggaagacataaaataaccccgcattttttaatgcttctagtcagtgaaggctttacaagcactggggccctcagccgctcagcctggtgccccgcggctgcggccttccccggggagggaccgaggcagcagctgggcctgggctcggaaaagcggcgctaacagggctcttcctttgcagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccagggtaagaagctggcgggggggatatcatgtggacaaaccgacagatgggcaggacccctccccacatccgtcattaactctcagattcaacgggggtaaagaaggcaagcaaggctgtatatgcctcgcagctctggccagggcctcaagattcagatcttcagacaatccatgtagctgggggcatagacatgaggacaggatggaggaaggaggagagggacacgccacagggtttgaagctgtgtgaattcccactaccccactaccccatcgcccctcctcttccatatacaccagtgcctctaccatgaaatccaggggctgtgcaaactctcccccttcccaatctactttattcccagtcctccatagagatagatgctttaatcctcatccttcctggcactgtgctggggaaggatgtgggggcctgtctgggggtcagggaagggaaggagagggtgtaaagaatgccagtggggtgggggatcaagtggtcagatcctttttactccagctgtgaaaaatatgcgggctttaattggaggaagtatgttgagcaaacctggtagggactgcaattttattaagatttgcaaaagggcgtctcagctcgaggcccactctgggactagcatgaatactaacatgtcaattgttttgtggagataagagtgaacgtttcccagggctggatggcactgtatttagtctgtatggaaatggcaatttacatatttaaagcagcgacctcgtagcaccatccctaattgaattaattgccccggaacatctaatttccttactggtcagagagaggtttaattgttataaaaacctggctcccctattagaaacggggttagcaatttcacgggttatatattttagagaacctcattaagtgctttttaaaatgaaattccagttccaggcgaaccctgactatcaaatgagtggagatgacacccagcatatccagcaattctatgatctcctgactggctccatggagatcatccggggctgggcagagaagatccctggcttcgcagacctgcccaaagccgaccaagacctgctttttgaatcagctttcttagaactgtttgtccttcgattagcatacaggtaataagggagggagggagacaatccagggaggctgtgagagaaatcaagaaaggaaaagaaagggaggaagggaaaccagagggtggggtagagaaaaagacagaataggaaatggaagtcggagaaaggaagaaaaagaaagaaaacaaaaaaagacgagaagaagcgagcccagaagccttggatgaatggaatggaggtgggatagggggcgttcttgattgttatgaaattaaaccctttcaaggtccactggtctacattttattaactcttcagtaattaggtgactcttaaatccctcatttattgctcttcaagtaattagttgtttagcttttctctctctctttttctcccctctctctctttggtattaattgcaggtccaacccagtggagggtaaactcatcttttgcaatggggtggtcttgcacaggttgcaatgcgttcgtggctttggggaatggattgattccattgttgaattctcctccaacttgcagaatatgaacatcgacatttctgccttctcctgcattgctgccctggctatggtcacaggtcagtactgcaggcgcagggcgcttcccctccagaactgcctagcaggatttgtcctgagtttcccttgtcacagattctccttggttttgccaactagctaactgtcttgtacattcttcttttgtttctgattatgttttctgcagagagacacgggctcaaggaacccaagagagtggaagaactgcaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaagacctcctcccaagcacttcaaaggaactggaatgataatggaaactgtcaagagggggcaagtcacatgggcagagatagccgtgtgagcagtctcagctcaagctgccccccatttctgtaaccctcctagcccccttgatccctaaagaaaacaaacaaacaaacaaaaactgttgctatttcctaacctgcaggcagaacctgaaagggcattttggctccggggcatcctggatttagaacatggactacacacaatacagtggtataaactttttattctcagtttaaaaatcagtttgttgttcagaagaaagattgctataatgtataatgggaaatgtttggccatgcttggttgttgcagttcagacaaatgtaacacacacacacatacacacacacacacacacacagagacacatcttaaggggacccacaagtattgccctttaacaagacttcaaagttttctgctgtaaagaaagctgtaatatatagtaaaactaaatgttgcgtgggtggcatgagttgaagaaggcaaaggcttgtaaatttacccaatgcagtttggctttttaaattattttgtgcctatttatgaataaatattacaaattctaaaagataagtgtgtttgcaaaaaaaaagaaaataaatacataaaaaagggacaagcatgttgattctaggttgaaaatgttataggcacttgctacttcagtaatgtctatattatataaatagtatttcagacactatgtagtctgttagattttataaagattggtagttatctgagcttaaacattttctcaattgtaaaataggtgggcacaagtattacacatcagaaaatcctgacaaaagggacacatagtgtttgtaacaccgtccaacattccttgtttgtaagtgttgtatgtaccgttgatgttgataaaaagaaagtttatatcttgattattttgttgtctaaagctaaacaaaacttgcatgcagcagcttttgactgtttccagagtgcttataatatacataactccctggaaataactgagcactttgaattttttttatgtctaaaattgtcagttaatttattattttgtttgagtaagaattttaatattgccatattctgtagtatttttctttgtatatttctagtatggcacatgatatgagtcactgcctttttttctatggtgtatgacagttagagatgctgattttttttctgataaattctttctttgagaaagacaattttaatgtttacaacaataaaccatgtaaatgaacagaa

SEQ ID NO: 7 - RNA, 3472 nt, Homo sapiens
  source 1..3472: mol_type = mRNA
aagccacataaacaaaggcacattggcggccagggccagtccgcccggcggctcgcgcacggctccgcggtcccttttgcctgtccagccggccgcctgtccctgctccctccctccgtgaggtgtccgggttcccttcgcccagctctcccacccctacccgaccccggcgcccgggctcccagagggaactgcacttcggcagagttgaatgaatgaagacagacgcggagaactcctaaggaggagattggacaggctggactccccattgcttttctaaaaatcttggaaactttgtccttcattgaattacgacactgtccacctttaatttcctcgaaaacgcctgtaactcggctgaagccatgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgccagtctcccgcctctccctcttctcctttaagcaatcgccccctggcaccccggtgtctagttgccagatgcgcttcgacgggcccctgcacgtccccatgaacccggagcccgccggcagccaccacgtggtggacgggcagaccttcgctgtgcccaaccccattcgcaagcccgcgtccatgggcttcccgggcctgcagatcggccacgcgtctcagctgctcgacacgcaggtgccctcaccgccgtcgcggggctccccctccaacgaggggctgtgcgctgtgtgtggggacaacgcggcctgccaacactacggcgtgcgcacctgtgagggctgcaaaggcttctttaagcgcacagtgcaaaaaaatgcaaaatacgtgtgtttagcaaataaaaactgcccagtggacaagcgtcgccggaatcgctgtcagtactgccgatttcagaagtgcctggctgttgggatggtcaaagaagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccaggttccaggcgaaccctgactatcaaatgagtggagatgacacccagcatatccagcaattctatgatctcctgactggctccatggagatcatccggggctgggcagagaagatccctggcttcgcagacctgcccaaagccgaccaagacctgctttttgaatcagctttcttagaactgtttgtccttcgattagcatacaggtccaacccagtggagggtaaactcatcttttgcaatggggtggtcttgcacaggttgcaatgcgttcgtggctttggggaatggattgattccattgttgaattctcctccaacttgcagaatatgaacatcgacatttctgccttctcctgcattgctgccctggctatggtcacagagagacacgggctcaaggaacccaagagagtggaagaactgcaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaagacctcctcccaagcacttcaaaggaactggaatgataatggaaactgtcaagagggggcaagtcacatgggcagagatagccgtgtgagcagtctcagctcaagctgccccccatttctgtaaccctcctagcccccttgatccctaaagaaaacaaacaaacaaacaaaaactgttgctatttcctaacctgcaggcagaacctgaaagggcattttggctccggggcatcctggatttagaacatggactacacacaatacagtggtataaactttttattctcagtttaaaaatcagtttgttgttcagaagaaagattgctataatgtataatgggaaatgtttggccatgcttggttgttgcagttcagacaaatgtaacacacacacacatacacacacacacacacacacagagacacatcttaaggggacccacaagtattgccctttaacaagacttcaaagttttctgctgtaaagaaagctgtaatatatagtaaaactaaatgttgcgtgggtggcatgagttgaagaaggcaaaggcttgtaaatttacccaatgcagtttggctttttaaattattttgtgcctatttatgaataaatattacaaattctaaaagataagtgtgtttgcaaaaaaaaagaaaataaatacataaaaaagggacaagcatgttgattctaggttgaaaatgttataggcacttgctacttcagtaatgtctatattatataaatagtatttcagacactatgtagtctgttagattttataaagattggtagttatctgagcttaaacattttctcaattgtaaaataggtgggcacaagtattacacatcagaaaatcctgacaaaagggacacatagtgtttgtaacaccgtccaacattccttgtttgtaagtgttgtatgtaccgttgatgttgataaaaagaaagtttatatcttgattattttgttgtctaaagctaaacaaaacttgcatgcagcagcttttgactgtttccagagtgcttataatatacataactccctggaaataactgagcactttgaattttttttatgtctaaaattgtcagttaatttattattttgtttgagtaagaattttaatattgccatattctgtagtatttttctttgtatatttctagtatggcacatgatatgagtcactgcctttttttctatggtgtatgacagttagagatgctgattttttttctgataaattctttctttgagaaagacaattttaatgtttacaacaataaaccatgtaaatgaa

SEQ ID NO: 8 - RNA, 3294 nt, Homo sapiens
  source 1..3294: mol_type = mRNA
aagccacataaacaaaggcacattggcggccagggccagtccgcccggcggctcgcgcacggctccgcggtcccttttgcctgtccagccggccgcctgtccctgctccctccctccgtgaggtgtccgggttcccttcgcccagctctcccacccctacccgaccccggcgcccgggctcccagagggaactgcacttcggcagagttgaatgaatgaagacagacgcggagaactcctaaggaggagattggacaggctggactccccattgcttttctaaaaatcttggaaactttgtccttcattgaattacgacactgtccacctttaatttcctcgaaaacgcctgtaactcggctgaagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgccagtctcccgcctctccctcttctcctttaagcaatcgccccctggcaccccggtgtctagttgccagatgcgcttcgacgggcccctgcacgtccccatgaacccggagcccgccggcagccaccacgtggtggacgggcagaccttcgctgtgcccaaccccattcgcaagcccgcgtccatgggcttcccgggcctgcagatcggccacgcgtctcagctgctcgacacgcaggtgccctcaccgccgtcgcggggctccccctccaacgaggggctgtgcgctgtgtgtggggacaacgcggcctgccaacactacggcgtgcgcacctgtgagggctgcaaaggcttctttaagcgcacagtgcaaaaaaatgcaaaatacgtgtgtttagcaaataaaaactgcccagtggacaagcgtcgccggaatcgctgtcagtactgccgatttcagaagtgcctggctgttgggatggtcaaagaagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccaggttccaggcgaaccctgactatcaaatgagtggagatgacacccagcatatccagcaattctatgatctcctgactggctccatggagatcatccggggctgggcagagaagatccctggcttcgcagacctgcccaaagccgaccaagacctgctttttgaatcagctttcttagaactgtttgtccttcgattagcatacaggtccaacccagtggagggtaaactcatcttttgcaatggggtggtcttgcacaggttgcaatgcgttcgtggctttggggaatggattgattccattgttgaattctcctccaacttgcagaatatgaacatcgacatttctgccttctcctgcattgctgccctggctatggtcacagagagacacgggctcaaggaacccaagagagtggaagaactgcaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaagacctcctcccaagcacttcaaaggaactggaatgataatggaaactgtcaagagggggcaagtcacatgggcagagatagccgtgtgagcagtctcagctcaagctgccccccatttctgtaaccctcctagcccccttgatccctaaagaaaacaaacaaacaaacaaaaactgttgctatttcctaacctgcaggcagaacctgaaagggcattttggctccggggcatcctggatttagaacatggactacacacaatacagtggtataaactttttattctcagtttaaaaatcagtttgttgttcagaagaaagattgctataatgtataatgggaaatgtttggccatgcttggttgttgcagttcagacaaatgtaacacacacacacatacacacacacacacacacacagagacacatcttaaggggacccacaagtattgccctttaacaagacttcaaagttttctgctgtaaagaaagctgtaatatatagtaaaactaaatgttgcgtgggtggcatgagttgaagaaggcaaaggcttgtaaatttacccaatgcagtttggctttttaaattattttgtgcctatttatgaataaatattacaaattctaaaagataagtgtgtttgcaaaaaaaaagaaaataaatacataaaaaagggacaagcatgttgattctaggttgaaaatgttataggcacttgctacttcagtaatgtctatattatataaatagtatttcagacactatgtagtctgttagattttataaagattggtagttatctgagcttaaacattttctcaattgtaaaataggtgggcacaagtattacacatcagaaaatcctgacaaaagggacacatagtgtttgtaacaccgtccaacattccttgtttgtaagtgttgtatgtaccgttgatgttgataaaaagaaagtttatatcttgattattttgttgtctaaagctaaacaaaacttgcatgcagcagcttttgactgtttccagagtgcttataatatacataactccctggaaataactgagcactttgaattttttttatgtctaaaattgtcagttaatttattattttgtttgagtaagaattttaatattgccatattctgtagtatttttctttgtatatttctagtatggcacatgatatgagtcactgcctttttttctatggtgtatgacagttagagatgctgattttttttctgataaattctttctttgagaaagacaattttaatgtttacaacaataaaccatgtaaatgaa

SEQ ID NO: 9 - DNA, 45009 bp, Homo sapiens
  source 1..45009: mol_type = genomic DNA
gcgcagccgggagagcggagtctcctgcctcccgccccccacccctccagctcctgctcctcctccgctccccatacacagacgcgctcacacccgctccctcactcgcacacacagacacaagcgcgcacacaggctccgcacacacacttcgctctcccgcgcgctcacacccctcttgccctgagcccttgccggtgcagcgcggcgccgcagctggacgcccctcccgggctcactttgcaacgctgacggtgccggcagtggccgtggaggtgggaacagcggcggcatcctcccccctggtcacagcccaagccaggacgcccgcggaacctctcggctgtgctctcccatgagtcgggatcgcagcatcccccaccagccgctcaccgcctccgggagccgctgggcttgtacaccgcagcccttccgggacagcagctgtgactcccccccagtgcagatttcgggacagctctctagaaactcgctctaaagacggaaccgccacagcactcaagtacgtatgagattcgcccagttaataaggggacttgctaaaaaagttggcttgctgggaagcgtcgctcctgaaattgacaactttgagtgtggggttccctcccctgaccttgccgcccggctccggctttgctagcccagcggcccgtaggttctgatctgaaacttttcccctgtagtgggcccgcggggatctctggagctcgtgggattcctccccccactcgaagaggcgaaaagcctctaacaaagaggaggaccgggatttgtgctatagcggctccagcgatgtaattcagggtatttcggctctagttgtcatggtaatgatgctctcggcggcggcggcggcggcggcagcggcagcggcagggagttgcagctccggtgatgaacggcagtaattttcctgccttttaagtaggattgaaaataggagctctggtgggtccaagtaaatgttgctaatggtggcaccgagctggttctctggaaggaagcttaggagggaagggcctaggcagcgacggccagagtttgcagacgcactcggaggacctcaggaaagagggcgtaattgtaggaatgtggcttattgtattgaaatgagcctggggttccactaggcacgtctgcctgtcgtcgctgacatgccggtttacacttttcccttaggaggtaaatcgggtgtaattggcgactcccgcacactgacacgtgtggggacggtgtccctctcctctggacgttggctcggtgtgggaaaggcatgctttttcacggacagaactcgcgcttttggagaagttgctccgagtgttttactcttagtagaaatgaaagttctcggtggtgagacgagagcggtcatgcgagcgcagcctgcggagactgacttccaaaggcacctctccaacctgttggaagcctcggggcagtggtgaggaagaacgcatttgtcccttgatttccaaaaacgttcttggagattgccttttatttatttatgcgtccggattctccagagggaagagctgcgaggagttagtaatcagaaatgtcgggctgtgatcccagggccttgtggtgctgggagagaagaacagggagccgtcattccatcgcgagtccagtgccggatctccttttcccacgaccgggaaatattttatagaatcaagttagggaaagaaaaagctgcctaacgtggctgatgggcacaatgaatgaagttactttattcgagttattttcgctgaacaagatgctcgcagagttagtatcaaagcctccttaggccacagcaacctccacgtccgctccgccccccaccccccggcacccatccacccctttgcctgggaccgtgcgctcacagaaattcttccccgcggtttgtctagtctccctcggtctttctgagaatctcagtctttccctttctgtcagtctctccctcgctcctcttgcccccgcccaacctccgcccccgctccccagggttggaaactccgcagaggagccccggtcacaatggtgcgtttcacggtttcctccacacacttcaccaccgggaggggaacacgggggtgttcctttcgtctgcccacgagaaagaaaggggctttgacagaggtagttatttcttcctaatttacaaccccaaggctctgtgctcccggccccggcggccgcaagcaggagcggcggaacgtgtttccaggcactgaggctttgcctaggtaaccggccgcttgtggggtggagagagcggcggggcgctgggctgggctggaggaaaggctgtgtgggtccctcccgcgaacgctgggcgctcgaggggaactccttcgttggggagaggcggtccccgcccacccgagttctgaggcctcctctcagagaacgactccgcttgaaaggccctcagggaatgcgggacgggaatgcgggcacccactgaaggggcgggtgggggattgctggagacccccattgaagatgcggggagacgggaagcacgtgctacagccgtgggaagatccgcttctacccgcgctacatcgagcgcaagctttccgtccgagcagctccagctggggctgggggtggcaagggaatcccccatttcctggtcgtcgagggtttcctggacctgggagccccgagtccctttctctcttcctgcaggcaagccccgggctcaggggcgccaggagatgaccctcgtggccccgcgcacctcggaggtttttggtttggtttgatttggggtttttcggtccctgttcccactcttcgagctgcgcccaccccgggcaaagggggcgctataggccggagtttgggcacccagttccttccctgaggcctgctgagctgcgctttcagctacaaagtttcgctgaggctgtgggctgagacgctggtcccggagctgcgctcggcgccctcaggaatgtgcccccgctaggccgctgggtggccaggagcctccaagccgcccaccctcgaagacaccgccctctgggtgcaggggacctgcctccgctcgtcccatcagccgctaacgccgtccgcttcgtccccttgcttccagcgccagccttctcatcgctgtgcccttttgtttgcttgacccctggccctcgaaactcgcggctaatagaagcgaagctccattagcatttagaatgaaaagcgcagactttcttaattcctcggggcattcatgcattcgttccggaccttgtgcatttcctatgcaacgtgtaaaatttgtatttgaggggtgggggcgggcggagctggaaaatggcttttttccgctggtgaacactcactgaccccccgtattcgggcggagctcgttcctcaagtgttctgctttctttggttttcctgacttcgtaagctcggaatcaattgtgggggcagagagatcaatttctgggcaataacaggctgcgcgttttttttttagcctcggttttggtgccacaaaacggtggtaggcgcagtgggttccaacaacctttcctcggcttccccgaggccgtgtgatgcttcttgcccagggggactcctgtaccattaatcacccttaattttacgataattctttgtaattaatgtaacatttctagaggttgccaaccaattcccccagtccccaatgcgcccagcccgggccgtagtgacccatcctggtctcactgtgacctatgtcctctttcaccttgcctgtagagcccactgcggaagagggcagcccggcaagcccgggccctgagcctggacccttagcggtgccgggcagcactgccggcgcttcgcctcgccggacgtccgctcctcctacactctcagcctccgctggagagacccccagccccaccattcagcgcgcaagataccctccaggtaggtccgaaggcaagacccttttctcctccctggctgagggaagtgggtgggggaaccacacactcggcgggcagcgtggtcgacctgcccagtgccaggacagtgactgctggccgcgaatttcacaacacaggtggcttcctcacaggaagctcctctgtataccacaccctgttgctactgagtggagcagccaaattaaattaagcttgcattgctcaaaattaattttcctaagagaaatacaaatacaccaatagattagggtattttatacatttttaatttcatttttgctcttcttttatagccagtgtgcacatgtaagagttatggaatcacttagagcaatactgagcattttcatttatataaaacccaaatcatttgggtgcagaagtttggatgattgaagctcagagggaaggagaaaagcatttgagatgaaaaccaaagagtaaatttgagtttggcaaaagaacagattgcacttttggtttgtaccacctctttacaaatttgttaaaggaatactaggtgccaagcctgtgtgggcaccatagataatgcagataaatagaatacaatcccagcaagctcatcttctagctagaaacctcagaaagacacaaataagagcagtaaaaagggattggccggaaagaatgacagaaaagagtaatggagaaatcctagaggtgctgaagggagaaggagaatagatcaaagatcaggcagtgttctttcttttaaatgattagcctttcatttcatcccaacaactggacagcaagataagtaactggacttcaaactagtgagtgtatttttaaggccctgcttgttaaagaaaggcttgaactggcctctcctcatcactgcttcttccaacaggccctcatcaccttttttcaagtcaagatttcatcccatacatgcatgactcaatcagatttggaaatgtgggtaagagaaagatgtcaaaggaaatgtgaagtattcacttctctattagtcacaccttttacaccatagactccaaagaggcgttaagcacctggttttcctttggctcagaaaaaccaaccaccaaaaaccgccgttttttaccatttatatttagccataaagaaagaaaaataattagataaatcatccactacatccaataattctcagcgccttctcactcagttcagcctctctgaacaatagtaagcaccctggataccagccactttgggggcaacatagtcaaactggcagagaaatcaagtctattgagaaactgctttttttcatgggtactaattctagtgtcataaggaaatacctatactaacttgcctattatgatagttataaactgttatcacaaaacagtcactgatatgttttattagttagaattgggatatatatatgtgtgtgtgtgtgtgtgtgtgtgtgtgtgtgtatatatatatatatgggtgggtgttttgttgcagctgctgatctttttctttgcagatggtacaaactctcccgagtcaatttcctgggcctatgtccccacctagctgactgaagttatcaacaggggtccagtttgtgcaggctgctagccctattggaagagtggggatgaggtgggagaaagcaaccacaacgtgtgtgggcaacctcaattggcactcataaaatgttagaatgtcaactctctcccttggccactaaatctctcacagggtagtttttcttgcctaactcaggtttacaaatcaatgtgtatgccttgggggaccaatggcctctttcctcccaaataaaccactggctttctctttgtccccctaggttatagctgaggagcccactccaattagtttataggattcaaagcctctttttaaaaacatctctgagcttatgaggaaagacttcaagtttcccaaatctagtggaggacagggcaagggaggaaagataggtacaggagtccacaggaggccaggttttggcacccctttgtcaggaattcagcttccttactagggatgaagaaaataagtgtggggctttgtgtctatgctaccagaaggaggagaggatgacacttcctctctgtttcccagattagagaacagtgaacccaatgctgcctgttggctagaaaacaagtgttaacttgcttctgagagacccttttctctgtccctgcagatatgccctgcgtccaagcccaatatagcccttcccctccaggttccagttatgcggcgcagacatacagctcggaatacaccacggagatcatgaaccccgactacaccaagctgaccatggaccttggcagcactgagatcacggctacagccaccacgtccctgcccagcatcagtaccttcgtggagggctactcgagcaactacgaactcaagccttcctgcgtgtaccaaatgcagcggcccttgatcaaagtggaggaggggcgggcgcccagctaccatcaccatcaccaccaccaccaccaccaccaccaccatcaccagcagcagcatcagcagccatccattcctccagcctccagcccggaggacgaggtgctgcccagcacctccatgtacttcaagcagtccccaccgtccacccccaccacgccggccttccccccgcaggcgggggcgttatgggacgaggcactgccctcggcgcccggctgcatcgcacccggcccgctgctggacccgccgatgaaggcggtccccacggtggccggcgcgcgcttcccgctcttccacttcaagccctcgccgccgcatccccccgcgcccagcccggccggcggccaccacctcggctacgacccgacggccgctgccgcgctcagcctgccgctgggagccgcagccgccgcgggcagccaggccgccgcgcttgagagccacccgtacgggctgccgctggccaagagggcggccccgctggccttcccgcctctcggcctcacgccctcccctaccgcgtccagcctgctgggcgagagtcccagcctgccgtcgccgcccagcaggagctcgtcgtctggcgagggcacgtgtgccgtgtgcggggacaacgccgcctgccagcactacggcgtgcgaacctgcgagggctgcaagggctttttcaaggtgagcgcacgccgccccctcccctccgcacccagccccctcactgaagggagcttctaagtgagtgtaggagcatccttgttcttcccggtttgagagctgtaatcggcacatcatgctttcctacagcccttcctagcaccttcacatccattttctcaggggctttctactgaggtccatgtgcagcaggccaccccagacggactccgggggtccgtgaacctcctggcctgacactgccctatgaacatttttctggagagctttcagcagagtcttaaaaggacctgtgacttcgaaaatgcaaaaacctcattgctttgtctgtccatccagggtgtctgaggggatagtgtgtgcgtaccaagccctggacatttgtgtctcatttaatccttacagtaagagctggggctctagagtcagacacatctaagattgaattctggccactcactagctatttttagtaacagtgttttaaaacagtgttttaattttctttgttttgtagatgaggatgttgaggtttgaagaggttaatcagctgcctcaggacacaccttcagagaagggagatggaggcaggatgccaacccacgtttgcgtgaccccaaagtccattcccttttcacagctctgcagctctaggagacagggacaactgtcttcttctatggaaagagctgcctctttccatagaagatagaacttggggtcttgtgtcctctaggaacctttccctacctctgtatcacatgcggtgccaggtgtagacataattcattccctcataggcctaacggagaaattttacagtgggagggccatcagcctcccccatattttacccacgaggaagctgaggcctggagcggtgaagccttatgcaggctgtaagcagtgatttaactgcccatgagatgtttccagctgatttgtcctgactcctgagttcagtgttatttctgccacttagggaaaagaccttgctcttttgctgtgctaccttttgctgttgactcccagactttatcagacacttacttctgtgccatccttctcactcctcaccccactggccattatttcagtcatttacaaacccacacaaattattccttatcctctagtgctggacagggtagaaagaaagagaaagcaggatgctgactgggtaacttggatttgggaggccaataggatatatctgggagtacaccagaattagtgagagaagactcaaatagaagaccttgatcttccaccttccaattgtatcatgttgggcatgacacttcattctaagcctgtttccctatcaatgaataaaactcagagaagagctgtgtattcaatgagataaaaatatatatgaaagtgctttataatctttaaagagctgtacagatagttcattattaaacaaaagctataaagttttgtaaaaatgcacatttgtggagggatatacatagcaacaagcaacaattatttttgatcttcagattgtaaatatcactacctgaattctgcagagaaatgagaaaatgtttggggaaagactgcaatacgttaagtgccctacagatatttgggattatgatgttttaaatgcagaatcttggtcattttattttattttttagccactgagtttggccattttttgttttaactactcaagctttatcagagctactttcaagtaactgcattcctttagcagatttgatttttacaggatttcagtggaaaatgctgaaggacagtgaaactggcagcagaaagggggaaagaaaaaaacaagaattatgaataatctacaaatataattatctctaaataatggagagttgaccaaaataccagagtggtaatacattctgagtccagaaatgtcaattaagtgttccccaacagcttctcttttctgcagcaatccctcgaaaacaaatctatacttgaaagggtgggggcaagatgggggagaatcaccccacagaactgttaattaagggaaccggagcaggttttacccagtggctttctaccattaaattgagttctttaaaaatgtgacatgaagtgcagttaatgataggaattcggtgattgaaccataatgatctctccctgtccttaaacacaaatgtagggaaggcttcaggttattgggaaaatgtatacctgaagcaaaatgccatcagcactcaaattggcataaagccttgaggatgatggcaagaaatagagtgttagaaattactgcatgttcaatgaaagctttattcaggaaacacacgtttcctacccttacagactgctaagaatttaaatgctggaacctcagaagtatgcctgtgtttcagaattttgcattttaaactaaaatcacctactatggtgggagaaacatgtcttagacttgacataggttggctttgttatattaagttttatataagcaatctagaaactatcccctaaaatgaaattaaaaggtaacagggtaaaactgaaacatgggggttgagaatgactggaagtaaatgttaaaaagcaaatctagatgtttgaacacaaaatagatcataaagcccaagttacctggatttattttctttttttattaatgcagcccaggcttcccaaataaatttcaactttctgctctatttggagttgtatagatccaggtaatttctgaacagtttcaccctaagtggtaaaaatacgttcatttgcacaaatgttttcaatttaaaaagcaagcatttgtttaaatttggaggacgaaatatgtttatttctccatgtgtcatttccaaattatatggattctgaaaaatacaacatcatttttctatccaaccttaagttttatgattttttttacaatatggttactgtgtctatgtcaatagacatttatgaaatgacctattaataggaattttcactgaaaatatatggctgtcatatatctttctttgtctcatttttcgtttgtaccttggggttttttgttttcttccttggatataataatgatttgtaataactacttttaagattttgctttgaaaaatcaggaatggactgtttatcatatatggtctttaaagataaaataagctatacaggctgattaagttagtttttttgcattaaaacattatttcctatatgttttaattagaaaaatcctgattttggtttttatgatttcatctctgttgggtaacaaagccagttactgtgatatttgacttacaatagtgatagcaaacaaagtacatcccagataattttgattttattttaataaggtcagcaataatcaggggagtggaacaagtagaaaattgttcccaaagggatattaaaagtggagataatctaattctccaaggtatgatatgttgacctggtaatttcagatatgattttatcattcagagttgggtgtctcctgagacttgctttagaacaatccgagatgattttcattgtcagcagctaacactaataaaattgtttttcctgcaggctagcgttttaggaaattaaatttatcgaattatacgaattgcactgtcgcttttcacattgtaattaaaatacatcttgtcaggtcctacctctttccagaaactatcagttgactatcttgtattatattttctcagagaacagtgcagaaaaatgcaaaatatgtttgcctggcaaataaaaactgcccagtagacaagagacgtcgaaaccgatgtcagtactgtcgatttcagaagtgtctcagtgttggaatggtaaaagaaggtatggatataatgctttttacccagtttgcttatacatattatcagaaatcagtgaaaaaaagctaatatttacattgggatggtgaatttttctagttcctttttcctttcaacattttatttttatcatatatatctacattttaaaataactgaggactatttattctaatttctcaaaattaacagaggacttgtatacatacatctttgttctatttaagaaggagacttttagggctaattcattaccaaatgtaaatatcggtggcttttatgctgcttgtttcaactctcatgaaatcattgtgtgaatattattccaaacttattaccacttttaaatggttggtctcattaatgattgctcaactgtaatgtgtttttattcctttttgcgatttatggtcgttcattccataatctttgaagattgttttctttgtctcttttggcatcagttgtccgtacagatagtctgaaagggaggagaggtcgtctgccttccaaaccaaagagcccattacaacaggaaccttctcagccctctccaccttctcctccaatctgcatgatgaatgcccttgtccgagctttaacagactcaacacccagagatcttgattattccagagtaagttttatgatttcctgctttcaaatgaatgatcagggtctctatttatggctactagtaataagagttgattgaatgattttgtgtctggcaccatgttagacagttttcatactttttctatatttctcgcttcatttagcaattcagtgcatccattgcagcaaataatttttgccttattgaatctctaaatgccttaacaagtgaccctgacagtgctgcacctgtcatacacattgttgcaggattcctggtggttgtgccaatgaaaatctgcacagacaaactacaatttgtagatttatctcgtgatctagacaaagtgactactgttttttttcatattgtgttcaaaccatctgggtgagcctcaagttattactaagcagtttatccaattgcatcagcattgattgacctgctgcttattcagagggattagaccattggtggagagaagcgagctctggctgtgacatcagttgtcaggagaactgggatggaacgccacctctgctacttcctgcctgtgtcacctggttcctgcttttggctctctcctgcctacagtggaaatggctacaatatttaacctcagaggggtgttctgaacaccagtcacacaagaatgatggttgtgaaaatgtttttaagaccataaatgacgattggtgttgttatagttaagtcaaccaaaacatatcaacatttttatttcatatgggatatagtaaaatcccatcagtctgaaccctgcaaattcctcaaaatttggataacacagagaagaaccatgagaaaggatttcctgagcaaatgaataaacagctctgtaaggcaatttctttaaaggacttagaaaactaaacttctagcaaaacagggtctcaatgaagagggaagagaaggaaaaaaaatcacaattattgagcagtttctatatgctagacattgtggtacattatctcatttaatcctttaaaccaccatgaattgtaggaatcattatccctcctacttgaaggaaaacaaaaacaaaaacaaaaacaaaaaaccttagaaacacagcttgcactcagataaagaaataccagtccagaggcccccagtcggttgatctgtcttggtggctactaccaaactgccctccccaggtctaatcagtgtttcaagcttctggctgaatggctggcaaacagtgtgagtgtgtccctgctatgtgcccttctccttaggttgcagtgttgatagaaatgattagtgtacctgctgtgatcctctttccctcttccccaccctcattataggctgcgaagcctcctctctgcacctgataacaaaacgtcatatgagaagcatggtagatccttagcatcaaaggttgaggactcttattctgattataagtagtggctcttgactacaatcaagtctcaaataatagtgtaagagaataaagcagaataataagactaagttaacagtttaggcttctttggaatcatgcgggcctagatgaaaatcccaacactgtcctttactagctaagtgaccttgagcaactgattacaccctttgatgcctcagttttctcctctgtgttgtggggtaatagtaatatctacttcctggggttgttcgtgaagattaattaacaattatacttgtcaaagctttagcacagtgccctgtatgttatttccttggccaaactttcttactctgccatttgttcaatgtcctaatgagcatgaacactacattaggtatcatgcagaacactctaaagataagtattatgatctctatttcacagataaggaaatttaaactgggagaggctaaagggctgacttgcccaaggtcactgaactaatatgcagcagagacagaattaggagccaagtatattaagagccaagtgtattgaacctaaaatctgggctcctaaataccaagcttcactgcctctctggttccagtgagagtgggtgctagaaaagtattcaggaatgaagagttctcctcccagaagcaccctggcctctccaaaggcagttcatctgaattagtgaagcttcttagcactgcctaggctaaggctatagcacacaatctgacattgctccagggcatttgaaagcacaatagatggtcagtacagataatttaggcctcaaaagtaattttttggccccaaattatccaattatcagtctacatggcctaattagtcattcaatggcaaacctttcccagcactttgagttgtcccagctgggaactgtccccacactgaaaaatcaatctagtgtgtttgcttgcctctgcaccattcagtctgttagtctagagtcatcatcctcatcaacaaacatttatccaatttaattgttagctttgtccaaaatagcagattgaccatgccagagcctgggtcacattggtactacctttggggcctggctcaattttgcaaagcatatttattttaattgcattattttggaatcaggttgaacctcataacttggattcccctgcttttagatggcattacacaattatcaaaaattggacccaaatcaactaaattaaaagactttttaaaaatacagtgagttttcatcaaaaacttggatgaaagcataaaagcatgctgatcacatttgcaaatgacagaaagctgggagggatcgctaatatgatggatgtcagtatcaacattcagagtgatctcgacaggttggaacaggaagggctgaaaccatcacaaagaaagttaacaaaaataaatctgaagctcagcatttagaatttaaacaaatgggagagccttggcaaagtggttcatgttaagaagatctggggagtttattccatatacactcaaaatgaaccaacaaagaacgtgtgtggtagaagggagggatgccacattaccttcagctgcagtagtaggagtatgggcagacccaagttgatgatggttgcatggactctgtcctgtcagactgttcttcgtgaattgagtccagttctaagctgggatttactgtttctgggaacttgtaatggatgattgggggaaacagaaatatgtaggcaagaagggaaaagatttggtgaaattgggtcactcttttgccctatggcaaaaagaactattctcccaaagacagatctagagagaggaggggggcctcttcccgctcacttgtctgataacaagtgttggccatactagctgctgtggaagtgtgagttcatcaacacagggagtaattaaggtagttttagttttagctttttggggaacccccatactgttttccataatggctatactaatttacattcccatcagcagtatatgagtgctcccctttctctacattctcaccagcatttattttttgtctttttaataataatcattctaactggggtgagatgatatgtccttgtggtttggatttgcatcttcctgttgatttgtgatgttgagcatttttaaacatacctgttggctatttctatgtcttgttttgaaaaatgtctatttaggttttttatctatttttaaatagattttttttctgctattgagttgaattccttatatattctggttattaatcccttgtcggatggatagtttgcaaaattttctcccattctgtagtatttcactttttaaattgtttcctttgctgaccatcacatatattcttacataatttcacttaagactccagctctgtgtggtattatttatggtttgagtaatttccttaataatgtcattcactgaaattcagttttcataataccttccttcttaaagttttacatatacaaattcataaaactggagtgaccttgggatcatctaatccaagtcccatattttcagaagagtaaattaaggtttaaaggggttaactggcttgcctaatgtcggtgacagagtagaggacaaaatccagatctcatcactcaataggtttctatttcatcatactgtcacttctatttaatgacaatttattatatttttgaacatataaactttattaaatatttttaaattacaataatagtaatatatatccattggggtttttttttaaaggcaaccatttacaagtggataaaatgaaaaatgaaagtctctacttcatttatagctgcccgatcccactccttagccgtgttttagtgtgtatccataatctattacattataaataagaatttgccctgtcaagtttgagagttgatttgagccctacaatttattaaagatgctagcttcccccagtgagtcaggtcaatagagaaaggaggaaatgtgttgccagcccagtatgttttaatcttcatgtactagcatgtgttcctttaaggtagtaagtgagcagttttctccatttgtgggaatgggaggtggaggggttttgttccaagtcacagaggaaatgaatgttttagaggccagaaagagctcctgagttctgtgaagcattcctggctctgggaccttcagttctcctttgggccaaacttctctccctcaaaagtgagctcagatcattgtacatcagtgcagtcaaaacatgtcacgtttgatttgtctctcctcaaaatgtttctgttctctgcaccacagttgcttttaatttagcaatacaagtatgtatagatactgggattagcttcagagcctgcataattactaatgagaaaacatacttagtgtcctcattagttgatcatttcaagtatttttcagcatactaggatgttttctggtaatcagtgccacttaacggagtaacagacttgcaaatacgagaggtgctgctcagataaaaccaggcaccagcaaatggcatttaaatgtgttcacaagatagcctatgaaacaatgtggcttaagacacgttagaactacaaggtaaacagaatgaagctgcaaaaagagtggtttcatgaagcatctcagaagaccctctcaaattgttaccaattcaccagatgatttcacgccagttcctgcccctctgtgggccagtttcctgatccaaatggtaaagagattaagctaaattcattcccattatctctccaggcttctcattccctgatattctgaccataatattaataactgctatagttccactatgagtacaggtcagcatttcctaaaacatggtgcccagactgctcctacgagaaagaggtggggtgtttgctttaaaatgcagattcccaggccatacccactcagacatactgaatcagaactgggaagaaggagaggcaggcaaaaatctgtactttaaaaagtgccattggatgattctttggcacactaaggtttgagaaccatcgatatagtttataataacaactcaattttaccttgaattttccagcttttcctggggttgagaagggatgagcaatagagatataaattttcctgaaagcaatcaattcatttaacaaatacttactgaatggctgctaggtagtaggcactgttccagggcaatggacacgttgctgaacaagacaaagcccttatccacatgaaccttacatacctgtaaaggagaaaaagagtaaacaaatatacaattgcagtgatgtcattggtgggaggagaggaattttttgctttttgctttttggagtgggggcatagagttagatcagaaaagaaaaaattggggggaaaatatattcattgccaatttttaaaatgtcactttttaaagtgtaagaacctaagaatatgtatacatagtttgacttatacaatgatcacatctaaaatttttagagctatagttgagaaaagtaacattttaaggggagaaaaacgtgtccttagcgtagtctacatatttagccagggctgaaagtgagatagagtaaatattagattccactctgctattaaagcctcacatcactaatttttgaggggtggtgttttccatgggtctcacttaatttccacacaaatatctcatttggggcctgggctattgctgaagtctgacttgtatagctgcgttactgccatatgaaacacacagacccattttagtttacataatatccattgctgttgtttgcagctctagattcccattctaggtgctttagagaaaccttccttaggcattggctgtcagtaaatgtaatactgtgtctttgactagtgagaaagccagagttctgacagatcaataacccctatagggtggaaaaaaattagtataaacaggaaaaaagttcatttaaaaaaatctttttgcatttgacctatgttcgattggcatgatcagtaagcaaatatttctagattttctttgtcaaaccccaaacctacttagcccagagacagagcaatcaatgtagggcagcagagacacagagctgggagtccagtccttccaactctaggaccagtattcattgggtgaggttttcctaaactggtaggccaggcagagaaaaaatctaaaacgttttgttccgttcctttacatcttatgtccaatagaggagatttttcttttcctccagcattggatgctgaccctccagtcacccccaagttactggtggctcagactgaattcactttggctccaaaattctgagacttggaccaaaaccactgcaggtgaagcccagaggatctggctggagcctggcaggctgggccggctggctttccttcttgctgggctccatcagagaaaagtacacacacagggtgggcagggacttcacttccctgtgtgcagaaggcatgaaatgtgagcccagcaggggcagaagcctgcagaggaccctgggtgaaagctacacactttgatggattctgaacaaatattggaagcagagagattgttgagttgtgagccatggattcaggggagtcagtgcaggaggtagctgtcagatccattctcaggggaaactattcattctttagtctttttctctctcccactattttaaaacaaaataatgctgaatcagtgtcaagttccaggcaataacatcatacctggtgtgatttagcaatatttagaatcatttaatgcaagagccagaagtaatcttagggatcaggtagtccactttattcctgttccagagactgaaactgactcagagaggttaaatgccttgtctagaactgcacagcaagtcagtgcagagcctggatgaggaccccatgacctgctgcttggtccaacacactttcctttactcccactcatttgggagtttcacaagtagctccctcagcttttgaaagggaggatctgccctgaatttcattctgctcttggagagcctgtggaattattaaataaattcataaataaggagaaaataaaaaaggggaaagttaattcccttttccccactctcttccagttaagagtctgtgtgctggtggggcgcggtggctgatgcctgtaattccagcactttgggaggccaaggcaggtggatcacaaggtcaggagttagagaccatcctgtccaacatggtaaaaccctgtctctactaaaaatacaaaaattagctgggcatgttggtgtgcgcctgtagtcccagctactcgggaggctgaggcaggagactcgcttgaacccgggaggcagaggttgcagtgagccaaaatcatgccactgtactccagcctggcaacagagcaagactctgtctccgaaaaaaaaaaaaaaaaggaggaggtagtggcaagtgtctatgacataggatcgtgccgccatgcttccttctcctggactttgctcccatggtctattacaacacagaatattcaggacttcatagaatcccaggcattgtgtaggattgttgggaacagagaagccagggctctgaactggaccagatgttataggatcttagaacatcagggctgaaaatcaaaaactttctacttcaacccattccatttcataaatatggaagctgagggatggtgattatttcagacatcataactgctatcaatatcatcactgtcatttgttaatcatgtactatatatcaggccctttacatatgttatcacatttaatcatcacagcaaaattttgaggaaggccagggatggtacacatggtaatcccagcatagattgtagggaggtccagccaggtggatcgtttaagcccaggagttcaagaccagtgtaggcaatatgggagttcaagacctgcctaggcaatataggagttcaagaccagcctaggcaatatagagacactgcctctacaaaaaatagaaaaaattagatgggcgtggtggttcctgcctgtggttccagctactcaagaggctgaggtgggaggatcgcctcagctggggaagtcgaggctgtagtgagccctgatcacaccattgcactccagcctgaggaacagagcgagaccctgtctcaaaaaaaaattttttttgaggaaggtatccccattttacagagtagaaagctgaggttcataaaagcaaaatgatcctacattatttcccatattaggggccagaacaaaattgagattaaagtagctggactctctgatttttttttttttttttttagttatttaatactgcttcttgggaaaacacaaagaaaaaaagacccaattaaataagcgtctcatttcctctcacccaggtagctccacagtaagcacctagttgcttattagctgtttcttctccaaccaggttaggatattacagcaaacctaagcactcttctgactattctttcattgaaaaaatattcaacagttatttattgaggtgtaattatgtgccagtcactgtactgttgacagaaacataggaccaatgaatacatacctggagagctccagtgagagtaaagcaaaatgctttttaaagatttcataggccagacgcggtggctcacgcctgtaatcccaccacttcaggaggccaaggcaggtggatcacttgaggtcaggagtttgagaccagcttggccaacatggtgaaaccctgtctctactaaaaatacaaaagttagccaggcatggtagcacacacctgtagttccagctactcgggaggctgaggcacggaatcgcttgaacccaggaggtggaggtttcagtgagccaagatcgtgccactgcaccccagcctgggcgacagagcaagacaccatctcaaaaaaaaaaaagatcccaccagctagtaacatcagacatacttaatagctaggaggggtaaaacttgaaatcacagggtcagcataagtcctggctgtaccactattggttgtgtaacttgagaaaatctcttagcttctctgagcctcagttttttcacctgtaaaataaagattaaaatccctacccatgaggatcagttcagaaaaggaatgcaaaatgatgcacagccatgttaatgtgaaggaaatgatgtgcctatttacgtgtttctgtgtatctcttaaatctctgtgttgttggctgtcccttgattagtatcttgctgtgtcagtcttcctgcttgcagcagaaaagtctttactccggtgattcaagaaaaactctggttccaccagaatttctgaaagctgggtgaaaactttattcctacttatatcgttctgctcttcggactctctagatccatgctgtccaatagacctcccctcagtgatggaaatgttctatcccacatttggccattgaacacttgaaatatggctaacatgactgaggaactgaatttttaatttcatttagtttttattaacttaaatagccacaagtcctgagtctgatggcacacagctgcctgggttctgccaaagagacctctctttccaccacttcctggtttgttttatctcacagaaattcatcaacagccagcaaaaatcattctttctcttgttgattatttctgcgattctcaaacttcattgtgtatgagaatcacctggagatcttgataagacagtttggtgggccaggcacggtgtcttatgcctgtaatcccagtacttcgggaggctgagacagaagaattgcttgagtctaggaaatcaagaccaacctgagcaacatagcgatacccacccccaacccacgcccccatttctacaaaaaaaatttttttttgatgtttgctgtgtctacccccagagtttctaattcagtaggtctgtggtagggcctgagattgtcatttttttttttttggagacagagtgccactctggcacccatgctggagtgcagtggcacgatcttggctcactgcaacctccaccttgcaggttcaagcgattctcgtgcctcagcctcccgagtagctgggattacaggcacatgccaccacacccagctaatttttatatttttagtggagatggggtttcaccatgttggccaggctggtctcgaactcctgacctcaggtgattcaccaccacccccccaccccccagcctcccaaagtggtggaattacaggcatgagccaccgcccaggccgaaattatcacttctaacacattcctgggtgttgctgatgctgccagtctggagaccacactttgagaaccactgggttaatttagcatctcatggggagacagctgtgctatagtgaaatgagtagacccttgagatctacttggacacaaactcctggggcagtagttctcaattgggctactccactttggagtcacctggggagcttttaaaattcttgacccctgggctccaccccagaccaattaaatgagaatctctaggcgcaggacccagacaagagttgtttcttcaagctccccaggtgattgcactgcacagtcaagggtgaaaaccactttgccaattcggcttaagcaaatttccttgtctattaagtgataacgataaaatttgctgtatttgtatcacagggccattgtgagaatcacatgagactgtgaatgtgaaggggttttataaactgtaacacctaaaagtaagaatttaggcattgatggtcttggaagggcctgtttgcaagtgatgccatcatccccactgaagcaggataatgctgctctctctggtttgcattctagtactgtcccactgaccaggctgctgcaggcacagatgctgagcatgtgcaacaattctacaacctcctgacagcctccattgatgtatccagaagctgggcagaaaagattccgggatttactgatctccccaaagaagatcagacattacttattgaatcagcctttttggagctgtttgtcctcagactttccatcaggtaattactactattttatcttcagtctacgtcctttgaagaagcctgaaaccttctgtgtttgtaactgaatcattggtgaaacgttttctcaagaagcaaatgtgaaatatgatcaggcactaaaaagactgagcagcgagtcacggagggagcactaggcttgccaccaggacccgtgggttctagtcccagttctaatgtggacttgctgtcacaccctgagcaggtcacttgcctgacgggaagttcaattccatcctccttaaaaggaagaggttggatttgatgatctcttgaggtcccttgcagctccaacattctatgattctcgaaagattctcatagtgcataagctatttcctgggagcaagtgtttgctttggaacattgtatttacctcgtgggtttctgtttaacaagttactattctagcacacctgatggggccctaaagagttcccacaccttctcaacccccagttttattccaagagaacactcttagatagaaagatttagtccatcctatcatactcagtgacgccaaacacaaaaaggaaaagtgatttttcaaaaacatagaaccttggatctaagctcctctagtaagttccacatgtagattttttaaagtttattttattattgtttttattcacaaatagcctgtttaactccatcttccttgcttcctttcagttatatatttttaggagatgataccaactttttaccaaatattatcagaaaaatccatctttcccctctttctctttcctgccatcacctccccaatttcatatcactaccagaaactcctgtttaatttttaaagtgcagattgccaggcctcattgccatctattttggtttggtagactgtggaagtccagaaatctgcatttttaacatatgctttaatgattctgatgtcatcagggtccatgttttatactctgagactaaatcattctgtcctgtgtatgccttctgtggagggatcagtaggagagatggggtggagttacaactagagtgccagtaatagcttaatttgtagccaatctagtgtacaccagagctgagccagcaatgtgactgtcatctattaggcatgcaacaaaggattaagtagactgtaagctccatgcaggcaggtgccatatatgtcctattcaccatgttttctcccagtgcctggctccaggaataacacatacttcatgctcagtagacacttggctgagtgaaggaatgaatgaagctgttcatccaagaagcacaaagagatatttgacttgtgaatacaacaggccaagatagatacatacgtggcaagagatttgaaagttaaaggaaatctaagattaaaggaagaaccacatataattcagccccttacttgttatacccaatatttgtcgaaccatcctgtgtactgggcaccatgcatgggctttcaaatcctttcttttatttaattccctcaacaatcttaggaggtagcaattagaatttttgttttataaataaggaaaaagaagtccagagagatttaaggagcacccaaagccacaccccaagtcatcatgtccacctggaagttccagggcacctatcatgaaagaagaatgagatgatgatggacataatataaactatcaggacagatttagaaatagccctctgcagccccaatgttaaaagcagagaaggtaatacaaaataaggacttgttttatgttaggccaccctttaatttgtgatagacttctggcacttacagggacttgatttttgttttgttttgttttgagacagagtcccactctgtcacccaggctggagtgcagtggcacgatcttggctcactgcaacctccgcctcccaggttcaagtgatcctcctgcctcaagctcctgagtagctgggactacaggtgcacaccaccaagcctggctaatttttgtatttttagtagagattgggtttcactatgttgcccaagctggtctcaaactcctgacctcagatgatccacccgccttggcctcccaaagtgctgggcttgcaggcgtgaggcatcgcacctggccagggacttggtatttttaaagtgtgcacaatggaattgtagtgtctagcctcaggccttggcagtttttaacatttaagaaaatcaaggacaagccctgtctcaggatccctggagggaacagtctcatgttcattgctgagagaggggtgcactgggcttcatctctggaacccatgagtgcctgctcccccatttcctgtcccaaggaaagccagtaattgattctagtaataagatagcaggtgccacagcccatatcttcccctgggctgatgttcaccaaagacattagcatagacaagtggtgctagttgtttatcccagagaacatagtcttattcattgtgggtatctggcatgcttcatagcaccacactggggtcccaatgctgccccatggctgtctgtgtgcctgtgtgtcataatgttatcatgttggacagattgaacagacctgtttaatgtttgtcttcctctcacctcccaggtcaaacactgctgaagataagtttgtgttctgcaatggacttgtcctgcatcgacttcagtgccttcgtggatttggggagtggctcgactctattaaagacttttccttaaatttgcagagcctgaaccttgatatccaagccttagcctgcctgtcagcactgagcatgatcacaggtaagcaccaccttgccaaaaccgcatcctcatttctctccttccctttgttatggtggaatctggccttgaccactacacacaccagaaaagtgggaaaatgactaccatttttctgaaatctgttaagtttaacctagtttaagcaaagaaacatgcatctataatttgttgtcataaacttctagtaaagaggatccatagtctattgaattatcattgtcagtaagcccaatatacgaaaatctaacttcctactagagatctgtggacagtgggactagctgcttcagggtttgtttgttttgagacagggtcttgctctgtcacacaggctagagagcagtggtgtgatcatggcctagtgtagcctcgaccacctggactcaagtgatcctcccacttcagcctcctgagtagctaggactataggcacatgccaccacacatttgtcctgttttgtagccctgtttcaccctgttgcccagactggttcagtgttcttgttacaaagcctttcctagggagattacctaacccagcccattcctgtggagcagaggtgatgaggtttcccctgtccccagttcttggctagcgacctaaagtaaaaccaaaacactatagacatatcaaatacaacttgatttaataacttcgattggcaaaggactgagaaaaagcaaaaatggttcaactcagcagttttttatttttatttttaaaattttattatcattatttttgagatggagtctcactctgtcgcccaggctggagtgcaatgatgtgatcttggtttactgcaacctctgcctcctgggtccaagcaattctcctgtctcaccctcctgagtagctgggattacaggtacctgccaccatgcccggctaatttttgtatctttagtagagatgggatttcatcatgctggccaggctggtctcgaactcctgacctcaattgatctacccgccttggcaccccaagtgctgggattcctggcgtgagccaccacacccagccaactcagcagttttttaaccacaaacataaaagatatttgatttatgcctgtctccctctatagatctctctctttctatcttttgggagataaaggatcccaaggtggggtgggatggagaaaggcgcgtgttgggcaaaggaaagaatatcatctccctgtggccatggaggcccttgccccagctccctttgccccgggggagagtttcctgttagacctcattctttctggtcagactggggcctggggaggtggtgacagagggtgggggctccagaagtggccaaatgaggggctcatccttgaccttttggtcagacacaagcacaggcttggctgttgaacagagttcaagatcagaataatatgctcttggctatagaaaacatgtttctcaagcaggtggtgagtttccagagcatcaaatgcaacctctggtacagatggccccttctttctgtttcactggaaatgtgcattggtttgcccaagcattgtttacttgcatttcttgggatatctcctattagctaaggttagatgtgtctgtgccttaagaggctaggctggttcatcatttgatggttgacatgcaggcaaagacaagcaactggtatggctgcaatgaggattcagacagtagctgttccttcagtattccaaatggaaactgacttcctcctggtctttggtccaggatgagaaactcactttttttttttaatggggctgctagacttccaagagagccattaacgaaacaaaaaccaaggcagtctcaaagagtatggattccatttggaaggctttcaaatggcatactggagcatctgaagaaaataaacagatttaaggcacaagcatattacttcatcaggactggaaggaaactggttcaaggctgcctttgaactaaagctctttagttcaatttcaaaaccattcagcaattcctacgtcagttaaacattcaaaactagaaagtatggagtttagttcattttaaaaagacaaaaacaaaaaactagatccagctttggattcctacaagtaaaagcagtacattgcttcatctccatggcaacgagtgttgagtctagttaatcaacaaatattcactgacctaggcaccatgctgcatgtcattgggttttcagagatgataacacacatccctagaggacatagacatgcaaagtaccacatgtgatgcaaggtgttgagtgccaccatggagacaagggagtgagttcaaagtgctcagagacagcaggtgggggtggtcttctcagaagagatcacaggagctggtcctttgtaagtgcagaggatgtctccaggctaaaatggaaaaagtgagaatccaggcataaacatttgccaaaagccattgaaaatatggctaagtttcagaaaggaaacagctgaaaattggagtgacaggagatgtgactggtctgataggtcagggtggggcgggacataaagaaccttccacgcctaggtgaggggagcagagtttatcttgagaacaacgaggatccacaggttttgtccaaaggagcttgatggtcaggtttgcgttttagaaagatttaccctgtcagtgagttttgggcatagattagagtgagaaagaatctaagaggaaaaaggccagtccaggtgtgagagccatgattcaagaggagggtccatccctgaaggccacatctttgtttcactctttaaatggaaattgacagatatctaagaggccaggacgtgtgggtgccaggaagacccaacctggagagactgggcagttcagtgcagagaatacaaagtagaaattagtagaagggtggtgctggtcctctttctgcatcccatttgttctattccctgcagttacagctgctcagcccgctgcccatttatgtggagattggctgggggaggaggcagtttggcatggcagaaatagcatggactttggaatcgagcacgcccagattttaatcttcagtcctatgtttaataagtaggcgctattggggaacacatttcagttctctgaaattcagtttccccatttgtaaaatggggataacaatgtctatgtcatagggcagcagtgaggattacagaaaatttagttggtgatgtaaccaaactggattcaggataggcaactctgaattcacctatggtcccccttggagtgatagtgacagattccctcaagttaggcatatgccttcccctaccccttggaagcatcaaactcagtggcatgaggaaacaggtggtggctttagaaggcagtatgtttggtgacagtctccttggatgcttaagagagaggctcaaaggaggatagtgatattaaagtaattaagctgcacttaacaagcacttacaatagaccaagtgctctacagtggtgtctttgggcctccccgaccaggtagctcctgttcctgccctcattcctgctgtggttttagcctacaacaaaaataagccctcttctctcatttctggaccttacaacaaacactctctttgtgacttactggttgaaagtgggtttgataagtgtgaaaatatcaggaaatggcagaaaaggcaatgcctgggaaacagtgtgcattgctgtgaatattaattttcatttgaatatgagctgccatggatcttgacttctgaataagcttcgttgatcataaatggtgattgtggagtatcagctgggctatttacattacaaaattcatcccaaaactgtcttttcttaaaccacagagagctcatgaaaaggaaatggcatcttagaagtttaactaggcttgcttccaggaaaagtattaagaatctggattagtattggtggtttgcacaaacatggcttttttttctctcattctttttttctttgtcctcctcctctctgtaccccctcatgaaaaactaggcggtgaagtggggcaggaaatggtttcattcagatcactctcctttcctgcagtaaacagggggttccccacagtggttggctttcaaacgtttttcaggttccctgtttatctaccctaaccattgccctttgccagtgctgcatctctgaagtaatgcaagtgctttgaatctggtgggataccaatgagttgatatctggttgcagttaataatgtttcataaataagaagccccctcaccaaacagaaagttagtgtggcattaccattttataaatatccactcattaaataagcatcgagggcccactatgtaccaagcattgtaccagtctcttgggatgcaaagatgagccaggcacgcattctgttgccaagaagtgggcagtctactaggaagtcatagattatgatatataaacttgatgtatagtgattagtgctcaggccctgaaaagggacagatgcagtgtagagtaagtccagattactgctaacttttttgaggtgaaggaaggtagaagtgttggtcatgaatgacttcccaaagaggtagcatctagacatgagaagacataaagaacattccagatgaaggaaagagaacagcaagggcatggcagttgaatgcctcagtacagtttagggttaaaacaattacagtggagttgcattgtgcatatattaaaatcacacatatgatacaagtagaaagaggagaaaaattaagagtgcaggctattgcatttgatgactgtatgtcctggagtgagcatgaatccactgtaggtcatggtttctctgcctctggtgatgagtggtgagatgatgtcttgtgcttttgggaaagttaaatgattttcagggataaaactaattctatgtgatatttaccttctggcttgatgtgatgacctaacctcagagcaaaacgcttttcctgtacagaaataggggaaataagattggaaagatagttttgaaccagatggcagagggaatctcaagtgctcaaatgccattttgttgttccccatggctttcttttccctactcaggtcatctctcaggtgagaaaatgactcctttctcgtctgactctagtctttttcctggtcttattgaaacacgacctctaagactgagaaggaggaaggagggcagaaccatggcagaaagtagtgaaacagtgacacagcaggactagacaggtggctgaccacggcacaccctccaccatctgtctttccagagctccactgctcactgtaaatggttacctggttacccctgcttttgcatcagataaagccatcagctacattgcaaggaaaactaaatatcaaacttagcggggcaaaatagttaaaaacaaaaatggtgggagaggggagaggccacaggatttagagaccggagatctgagttcaggtcttcaccctgaaatgtgctgtctttgcaatcttggacaagttccttaccctctctgaatctcaatttccttccttctcacagggttaaagatgtctactttcaggtttcttataaaaattgaatgaactaatatgtactatgcagaaaaaaaattagacaaaccttcactgttattatttgttttacacacttccatgaagagccagctgtcttgctaagaatccataaaagtgttcatgcatccctgcaactgaatatacacattgtgttagggaatttctttttttttttttttttttattatactctaagttttagggtacatgtgcacattgtgcaggttagttacatatgtatacatgtgccatgctggtgcgctgcacccactaatgtgtcatctagcattaggtatatctcccaatgctatccctcccccctcccccgaccccaccacagtccccagagtgtgatattccccttcctgtgtccatgtgatctcattgttcaattcccacctatgagtgagaatatgcggtgtttggttttttgttcttgcgatagtttactgagaatgatggtttccaatttcatccatgtccctacaaaggatatgaactcatcattttttatggctgcatagtattccatggtgtatatgtgccacattttcttaatccagtctatcattgttggacatttgggttggttccaagtctttgctattgtgaatagtgccgcaataaacatacgtgtgcatgtgtctttatagcagcatgatttatactcatttgggtatatacccagtaatgggatggctgggtcaaatggtatttctagttctagatccctgaggaatcgccacactgacttccacaatggttgaactagtttacagtcccaccaacagtgtaaaagtgttcctatttctccgcatcctctccagcacctgttgtttcctgactttttaatgattgccattctaactggtgtgagatgatatctcatagtggttttgatttgcatttctctgatggccagtgatgatgagcatttcttcatgtgttttttggctgcataaatgtcttcttttgagaagtgtctgttcatgtccttcgcccactttttgatggggttgtttgtttttttcttgtaaatttgtttgagttcattgtagattctggatattagccctttgtcagatgagtaggttgcgaaaattttctcccatgttgtaggttgcctgttcactctgatggtagtttcttttgctgtgcagaagctctttagtttaattagatcccatttgtcaattttgtcttttgttgccattgcttttggtgttttggacatgaagtccttgcccacgcctatgtcctgaatggtaatgcctaggttttcttctagggtttttatggttttaggtttaacgtttaaatctttaatccatcttgaattgatttttgtataaggtgtaaggaagagatccagtttcagctttctacatatggctagccagttttcccagcaccatttattaaatagggaatcctttccccattgcttgtttttctcaggtttgtcaaagatcagatagttgtagatatgcggcattatttctgagggctctgttctgttccattgatctatatctctgttttggtaccagtaccatgctgttttggttactgtagccttgtagtatagtttgaagtcaggtagtgtgatgcctccagctttgttcttttgtcttaggattgacttggcaatgcgggctcttttttggttccacatgaactttaaagtagttttttccaattctgtgaagaaagtcattggtagcttgatggggatggcattgaatctgtaaattaccttgggcagtatggccattttcacgatattgattcttcctacccatgagcatggaatgttcttccatttgtttgtgtcctcttttagttccttgagcagtggtttgtagttctccttgaagaggtccttcacatcccttgtaagttggattcctaggtattttattctctttgaagcaattgtgaatgggagttcacccatgatttggctctctgtttgtctgttgttggtgtataagaatgcttgtgatttttgtacattgattttgtatcctgagactttgctgtgttagggaatttctttgtgtctttctttaccagctagactgtggggttcttaaggacagggagagtcttatttatctttaaaattactttagcatttacaagtatctggcatatgacaaatattcattgaatgaatgggccaattgtgagtatcagaaagtctcccaaattagaaaaatctgaattaataatttttagcctaccactgttatgataaaagtatgaattcttgaaaattgccttattttgattcagattaattcagtaaatatttattgagcatcaactgtgcaccagacacacattcccttaccctcaaagagtctgtggtctcactggagagagagacagacacatactctaccaaatcaaagacggtcagactgaaaagtaccatttaaatcagcctcaacacagactgctgacttgacttgtcgcttcctgaccagcgtgggctcaatgtctctgggactgcccatcagtaaaggatgttgtgtatgacatgagtgatagcaataaggtgaattcccagagaaaggggtagagctccgagtggggagtcctgtttgtttgtgtttgagctacacatacctctctctccaggggcccacctttccttcccctttggctggaacagcattgttgtcaccaagttagcctttgccctaacacctgctcagaaaggggagttacttttccccttggtggggctcctgatggttactatcatccctttctttatcccctactccctgccatgaggaagagaatttgacatttctcatataagcccaagaggcagaactgggaactacatatagcagcggtccccagcatttttggcaccagagactggtttcatggaagacaatttttgcttggactggggtcggaggaatggtttcgggatgattcaagcttattacatttattgcgcactttatttctattattattacattgtaatataaaattaaataattgtataactcaccataatgtaaaatcggtgggaaccctgagctcattttcctgcaactagatagtcccatctgggggtgatgggagacagtgacagatcaccacgcattagattctcataaggaacgtgcaacctagatccctcacatgcacagttcacaatagggttcacactcctatgagaatctaatgccgccactcatctgacaggaggcggagctcaggtggtaatgcaaataacggggagtggctgtaaatacagatgaagcttcacttgcttgcctgccgctcacctcctgttgtgtggcccagttcctaacaggccatggaccaataccaattggggacccctgatatataggatggcaaattttagctcaatgtaagaaaaactttcacatagaaagaaccatttgaaattggaataagctgccttgaggagtgagttcctgctttggggaatgaggcttacagagactagatgaacactaggtgtggactcagtcaagcatcttatggggactaggattgactagagcagagcttctccagcattcacatcactgggcatcttgttgcattgcagattctggttaggtaggtctggggcagggcctgcctaaacggattctgcatttccagcaagttccccagtgatgctgatgctgctggtctggggaccacaccttaagtatctaggaataagggaacctattggtaattaggaatccatgaaattaatagctaccatttaaaaatactcagtgccaggcactgtgccaagggctttacatgtgttattttgtttaatgttgcttaataaataaacttggaagtacctgaaatagattatagcagatacaggaagcatggagatcagtcaagagctattaaaataatctagatagcaaagatattgaagatcctcatccagaggccatgggtattgagagggagatataaatgcaaggaacatcacagagataataacggaagttcttgcaatggattaaatatgatggctcggggggaagaaattgttagagatgattataagagtgatgtgaaaggctgtaatatcattaatagacaagcttagagacgtggctgatttgaaggggaaggtaatggcttgagttttagatagagaggatacttgcaaatattcactaagtgctcactatatgctaggcatggttccaaatacttgacatgtattaactcgtttacttctcataacaacccataaggcagctaccattattagcaccattttacaaatgtggaaacagacccaaagtcacccagttaataagtggtcgaggccaggggtggtggctcacacctgtaatcctagcactttgggaggccaaggcaggcggatcacaaggtcaagagatggacaccatcctggccaacatggtgaaaccccttctctactaaaaatacaaaaattagctgggccttgggaggccaaacaggagaatcacttgaacccaggaggcgaaggttgcagtgagccgagattgcatcactgaactccagcctggcgacagagcaagactctgtctcacaaataaataaataaataaataaataaataaataaataaataagtggtagagttcaggtctacagccaggaaatctaattccaaagctttgatgtggtaggttttagagtttgtaaagttctttactgttgttagcttcttaataactcccaacctgtatatttatttttagcagcttacacttttagtgcttatataatcatttctcacagattcttgatcctctatgtatcaatttaacagatatttattgggtgtctcctatgtactaggcttggtgctggagatacaatgttgagcaaatacagttcatgcccccatggtgcttacagttaatgggaggagaaaaaaatcacataactatctgacttcaaactgttgtgtcctgcaaagggatagattagaatgccagaggagaacatgatctagaataggggtaaaaagggaagcttccttcaagaaatatttgatctgagatccaaagaatgagtatatcttaaccagatgaggatgggaaaacatgagcacagacctaaatatagctctattatttcactccatcactctctccccctgactccttcttccctggttccacctacctcgcaacccctccctcttgctctaagcagagatgtctatgcctggatagccaaaatgcacagaaatccaacggtgtctttaggtgttcataccacacaaagattaggagtgtgatctatctgtcgcatctctaaccccagtggttataacatcgcttgccacagaataggtgttcaataaatatttgttgaaagaagtagttagttaatttggtgcacagaaaataggagagagttatagaaaagctagttcatctcaggacgaggagaaactttctaacaattagagatgtccagagatggaagacatcttgttataagagaaatatctctggacttagagtaaaagaaaatagccccgtgctaattttgactatgggcaagtcacttaattgaggcttaccttccatacctattacttaaagataatctttgtcctgcctaattcacaggattgttgtgagagtgagataagataatctaagtaaaagagtgttgtataaagtgctattaaatatatatgtgctattatggcataatatagttaactcccagtccagccactacctgacactctctttatggtagcttgtagtagagattcttgtcttagatagaaatttgaactaaataactcaaaccctttccaatctatatatacaatgattatgacacataagtctattacttaaccatttctgtatgttacatatcaaagttttgaatatgtaatgccagagttaaaatactaaagcattctatgaaaattatatagatcaaaatgcaagaaatattaagaagcttgttttgtttaatggtggacaaagaccttaaactaaaatctgctgtgtataattctctaggaagggggatagtccaaatgcctgatttccccccgaatttatgaactctgtatattagctccgtagccatgtttaccaagctacaagaagaaaggagatccctttaactaaaatgtcagtgattaggttttaagggatttcaaattactagggaaggacccagacacttagtgactccctagtgatttgaaaatcttcctggctgctggcagtagtttgcatggagactgaatattatctcagcccttttgaaaaagtaaagaagctacaaatccagtggctgttcaaaacaacaaagagggttgaggtgcctttggcttctaacaggactgtctctgaattcctcaaggaattccaggcctagagttgatccagctggcaggaggtatcgatctccacttgaattcactgaataaataatggttgccatagttattgagcagtcgcaactggttggagagcagggaaaattctgcagtaccttctgtgagtttggccatgagaaagtctggttacaaggttccacaatcagctccctccccaaatccagccaacatattactcagttcctgtatcctgatgatgaatatgagatcttgaatgttatgtaagacatgagacactgaaaggaagggaaagcttaaatcaatggactggtatttattctgagaaaggcgtctctccctaggttatgaatataattgaatatttttcattattaattcaatttagtatcagcaaggtgatgtttgatcatgttatagcattcaagggcttcacaaaggacgaaatccatctcgttctattttaacattattatacaaagagcacaacctggacattttaaaaggaaggaaaatttatcccaaaaccccataggtattttcacgtttatattttctttcacccttctctatatgaagaaataatttttgcatagatggaatcacagctttagtatttaatattctactctttatttaatgttgtaaacgtttttttttttaattttgcttcatggccttcacatttgttctgtttgatgtcttcagaatcttcccttccatataccattatttacttaacagttcccctattcttcagccctttgcctttttccagttttttgtttgtttgcttttgttttgttttgtcttggtaatagataatgctgaaccaaatgtctccatgtatacagttttattcttctgttaaatgtttcctttggataaattcccaagagtgggattacttggtcagaggctatgaacgatttatagctcttgaaatttactgccaaatttacagcctgttcttttgttttggggagaaaataaaactttaggcagaacaccactgcatgcaggattctcttaaaattcattctcttaaaattcacaacagtctcttctgagactgaaacatggagcctgtgtatttagtaatgataagccgtaggagacaggtccgcacctgggctgattagctaatagtttctcagggaaagcaaaatgatgcactgcaaagcttggaaattaggccacgaattgctttttgttagaccttgtagttaccagccacttgcccactggcaagcattgtctttggttctgattattccatgtgtatagcactctacagctgacagaccacttttccataacattatcttaattgagccacaataatccatggagttgggttttattatcattgtactttttcttttaatttattacataacattgaagacacacaggaataataacagcaaaggctgtgtatctgccactcaataattccgattttcctgtgaagacattgagccttagagaaattaggtcacccagctagtaaatgacagaaccagaactagaacctaggtctgtctaactccagggccagtgcccatctctttatagcatattgcattgagacaagatgcctgcaacttacagtccatggagatcccgctggcaatcttcatggttgtattttaatatcattataacatatcaggccaggtgcagtggtgcatgcctgtaagcccagcactttgggaggccaaggtgggtggctcacttgaggccaggagtttgaggccagcctggctgatatagtgaaaccctgtctctactaaaaatacaaaaattagccaggtgtggtggcacacacttgtaatcccagctacttgggatgctgaggcaggaggatcttttgaaaccgggaatgttgcagtgagctgagatttcaccactgcatagcctgagtgacagagtgagactctgtcaaggaaggaaggaaggaagggagggagggaaggaaggaaggaggacacatcagagctttgggaccttacagataatgaagttcaaccgtctccttttacagggccccaaagtgggaggaacttccccaaatcatacacaactagccaaactgaggataatgcaaattttctggttcccaggcctcttatttgggacaagaaaaaagtgtctggatagttcttcatttttcttattggcttttgcttcaaggaaataacttttctaacaaaaatgcccccaacttaaaaaattaatttagagccaggcacggtagctcatgcctgtaatttcagtgctttgggaaactgaggcaggaggatcacttgagcccaagagttcaagaccagcctgggcaacatagtgaggccctgcctctacaaaaaataatttaaaaatcaaccaggtatggtggtgcatgcctatagacctagctacttgggaggctgaggcaggaggatcacttaagcccaggagttccaggctacagtgaaccatgactgcaccactgcactgcagcccaggcaagagagcaagaccatgactcaaaaaaaaaaaaaattaattttgtaaaaattagaaactattgcttgatttcccattagaaggatgtcttccatctctcaagaattatgattttacatgtaaatgtactgagagaagaaaatttcgagataacagaggccaagctcttcctttagaaatgaggctctgaggccaggcacagtggctcatgcctgtaatcccaacactttgggaggccaagatgggaggattacttgaggtcaggagttcgagaccatcctggccaatatggtgaaaccccatctctactgaaaatacaaaaaattagctgggcatggtggtacacacctgtagtctcagctactcgggagggtgaggcaggagaatcgcttgaatcggggtggaggaagtgggggagcagtgagccaagatcgcaccactgcaccccagcctaggcaacagagtaagactctgtctcaaaaaaaaaaaaaaaaaaaaaaaaaaaaaaaaaagagagagaaatgaggctctgaagtctaaatgttgaggaaaagcatagtgatttgttgtggatgtcaggctcccttctcattgctttggaacaagagaccagtatttgcctcagaaaactgggttctaatcctagcttttagcagctgagtgactttgggaaattcatttaaccttatacataagatagatcatagtatttaacatggagatcattttatatattgttttgtaaaatataagatactggtatttgtgtcttaatacattataacatgcctgcaccacatccttctgggcttcctccccttctctttggcttttccatctccacagcaggaggacatgtgtattttgtacagagtgccaggtgtaggtcaacaggggtttcaagtgggggggaacaaaacaaggaaaagaaaagctgaattcttctcagtgctggttagcatgaaaacacaggaatgcactttgctgtgttgggtcttttcgagcagcccttgcattagcaaatggaagttacagcctccccacctcccttaaatgagtctgtcaagatagaaacattaattcatttgcctggatgtgtttctatgtcactctgaaaaatagttgcctactgacaaggctttgtgagagcttagtaggataatgtatgcaaaataataaccaccctgccattcattccatgtggctgtgtgccaggaactgtgcttcccttgtgttacttacctaagctggccatcccagaaaccctgaattgggtatgatcattttcttcatctcataaggaaatgggtgctcagaagtaaagagacttactctagggcacacagcaagtaagtgttgggactgggattcaaaccttggtgttgcttcataccaaaggccatgagctatctctaacagggagggcactttgattgcaaagaagctatccaaactggcagaatgagacacagacctcaattagggattcaaagtgtcttaagatgtatttgcctcctaaacttcttgcccttctctatccctctgcagaaagacatgggttaaaagaaccaaagagagtcgaagagctatgcaacaagatcacaagcagtttaaaagaccaccagagtaagggacaggctctggagcccaccgagtccaaggtcctgggtgccctggtagaactgaggaagatctgcaccctgggcctccagcgcatcttctacctgaagctggaagacttggtgtctccaccttccatcattgacaagctcttcctggacaccctacctttctaatcaggagcagtggagcagtgagctgcctcctctcctagcacctgcttgctacgcagcaaagggataggtttggaaacctatcatttcctgtccttccttaagaggaaaagcagctcctgtagaaagcaaagactttcttttttttctggctcttttccttacaacctaaagccagaaaacttgcagagtattgtgttggggttgtgttttatatttaggcattgggggatggggtgggagggggttatagttcatgagggttttctaagaaattgctaacaaagcacttttggacaatgctatcccagcaggaaaaaaaaggataatataactgttttaaaactctttctggggaatccaattatagttgctttgtatttaaaaacaagaacagccaagggttgttcgccagggtaggatgtgtcttaaagattggtcccttgaaaatatgcttcctgtatcaaaggtacgtatgtggtgcaaacaaggcagaaacttccttttaatttccttcttcctttattttaacaaatggtgaaagatggaggattacctacaaatcagacatggcaaaacaataatggctgtttgcttccataaacaagtgcaattttttaaagtgctgtcttactaagtcttgtttattaactctcctttattctatatggaaataaaaaggaggcagtcatgttagcaaatgacacgttaatatccctagcagaggctgtgttcaccttccctgtcgatcccttctgaggtatggcccatccaagacttttaggccattcttgatggaaccagatccctgccctgactgtccagctatcctgaaagtggatcagattataaactggattacatgtaactgttttggttgtgttctatcaaccccaccagagttccctaaacttgcttcagttatagtaactgactggtatattcattcagaagcgccataagtcagttgagtatttgatccctagataagaacatgcaaatcagcaggaactggtcatacagggtaagcaccagggacaataaggatttttatagatataatttaatttttgttattggttaaggagacaattttggagagcaagcaaatctttttaaaaaatagtatgaatgtgaatactagaaaagatttaaaaaatagtatgagtgtgagtactaggaaggattagtgggctgcgtttcaacattccgtgttcgtactcccttttgtatgtttctactgttaatgccatattactatgagataatttgttgcatagtgtccttatttgtataaacatttgtatgcacgttatattgtaatagctttgcctgtatttattgcaagaccaccagctcctggaagctgagttacagagtaattaaatggggtgttcacagtgacttggatacaccaattagaaattaaataagcaaatatatatatatatataaatatagcaggttacatatatatatttataatgtgtctttttattaaccatttgtacaataaatgtcacttcccatgccgttattttatggttcatttgcagtgacttttaaggcagtactgtttagcactttgatattaaaattttgcttatgttttgctaaattcgaataatgtttgaagatttttaggtctaaaagtctttatattatatactctgtatcaagtcaaaatatctttggccattttgctaagaaacaaactttgaatgtcaaactgatgtcacagtagtttttgttagctttaaatcatttttgctttagtctttttaaaggaaaataacaaaactatgctgtttatattgtcattaaattatacaatcaaacaaatgccaaatgaattgcctaattgctgcaaagtataacccagataggaaatcatatgtttttttccaagagtcattctaatatttgattatgttatgtgtgcttttatgaaagattgttatttttatatatcaagatgatagaacctggaatgttaggattttgaaatgttagacttggaaggggcctggtctgtcaactagtccaaccccttaaaattcatagaggagcaaactggggcccattgaagggtgaagagttactcaaggtcaaacagctggtaacagaatcaagactaagacctaatttacctttccatactctttttttttctcaacttcatctatataaaatcaggcttttaaacataaccactaatatttacctgaagataaccatgagtaaagtatacttttgcattaattttttgagcttatatgcaaacataataaatattattaaatatcaggaaagctaacatttcatacaagatagcttcagaccaaattcaaattgaatttgaataaattagaaatactgtgcatacataaccttcttgtgcaccatgagtatttggaaagttaatccttgtttttgtcgtgtctataaaggaagaacaaaacaaaataaaaacagagccctagagaaatgctgttactttttatttttacacccatcagatttaaggaaaagactttttagccattataatctagtggttggaaggaatgaagaagcttttttagtaataggtccagatatgagtgctaaaaataaagatgatagcatgttcttctgtcttccatagttattacaactatgagagcctcccaagtcatcttatcaactcaactcccttttttttgtcttaatgttgcacataagtttatacagagtggatgaccacactagcacagaagagaacaacatgtattaaagcaggtgattcctccccttggcgggagagctctctcagtgtgaacatgccttctgtgggcggaaatcaggaagccaccagctgttaatggagagtgccttgcttttatttcagacagcagagttttccaaagtttctctgctcctctaacagcattgctctttagtgtgtgttaacctgtggtttgaaagaaatgctcttgtacattaacaatgtaaatttaaatgattaaattacattttatcaatggcta

SEQ ID NO: 10 - RNA, 5604 nt, Homo sapiens
  source 1..5604: mol_type = mRNA
gcgcagccgggagagcggagtctcctgcctcccgccccccacccctccagctcctgctcctcctccgctccccatacacagacgcgctcacacccgctccctcactcgcacacacagacacaagcgcgcacacaggctccgcacacacacttcgctctcccgcgcgctcacacccctcttgccctgagcccttgccggtgcagcgcggcgccgcagctggacgcccctcccgggctcactttgcaacgctgacggtgccggcagtggccgtggaggtgggaacagcggcggcatcctcccccctggtcacagcccaagccaggacgcccgcggaacctctcggctgtgctctcccatgagtcgggatcgcagcatcccccaccagccgctcaccgcctccgggagccgctgggcttgtacaccgcagcccttccgggacagcagctgtgactcccccccagtgcagatttcgggacagctctctagaaactcgctctaaagacggaaccgccacagcactcaaagcccactgcggaagagggcagcccggcaagcccgggccctgagcctggacccttagcggtgccgggcagcactgccggcgcttcgcctcgccggacgtccgctcctcctacactctcagcctccgctggagagacccccagccccaccattcagcgcgcaagataccctccagatatgccctgcgtccaagcccaatatagcccttcccctccaggttccagttatgcggcgcagacatacagctcggaatacaccacggagatcatgaaccccgactacaccaagctgaccatggaccttggcagcactgagatcacggctacagccaccacgtccctgcccagcatcagtaccttcgtggagggctactcgagcaactacgaactcaagccttcctgcgtgtaccaaatgcagcggcccttgatcaaagtggaggaggggcgggcgcccagctaccatcaccatcaccaccaccaccaccaccaccaccaccatcaccagcagcagcatcagcagccatccattcctccagcctccagcccggaggacgaggtgctgcccagcacctccatgtacttcaagcagtccccaccgtccacccccaccacgccggccttccccccgcaggcgggggcgttatgggacgaggcactgccctcggcgcccggctgcatcgcacccggcccgctgctggacccgccgatgaaggcggtccccacggtggccggcgcgcgcttcccgctcttccacttcaagccctcgccgccgcatccccccgcgcccagcccggccggcggccaccacctcggctacgacccgacggccgctgccgcgctcagcctgccgctgggagccgcagccgccgcgggcagccaggccgccgcgcttgagagccacccgtacgggctgccgctggccaagagggcggccccgctggccttcccgcctctcggcctcacgccctcccctaccgcgtccagcctgctgggcgagagtcccagcctgccgtcgccgcccagcaggagctcgtcgtctggcgagggcacgtgtgccgtgtgcggggacaacgccgcctgccagcactacggcgtgcgaacctgcgagggctgcaagggctttttcaagagaacagtgcagaaaaatgcaaaatatgtttgcctggcaaataaaaactgcccagtagacaagagacgtcgaaaccgatgtcagtactgtcgatttcagaagtgtctcagtgttggaatggtaaaagaagttgtccgtacagatagtctgaaagggaggagaggtcgtctgccttccaaaccaaagagcccattacaacaggaaccttctcagccctctccaccttctcctccaatctgcatgatgaatgcccttgtccgagctttaacagactcaacacccagagatcttgattattccagatactgtcccactgaccaggctgctgcaggcacagatgctgagcatgtgcaacaattctacaacctcctgacagcctccattgatgtatccagaagctgggcagaaaagattccgggatttactgatctccccaaagaagatcagacattacttattgaatcagcctttttggagctgtttgtcctcagactttccatcaggtcaaacactgctgaagataagtttgtgttctgcaatggacttgtcctgcatcgacttcagtgccttcgtggatttggggagtggctcgactctattaaagacttttccttaaatttgcagagcctgaaccttgatatccaagccttagcctgcctgtcagcactgagcatgatcacagaaagacatgggttaaaagaaccaaagagagtcgaagagctatgcaacaagatcacaagcagtttaaaagaccaccagagtaagggacaggctctggagcccaccgagtccaaggtcctgggtgccctggtagaactgaggaagatctgcaccctgggcctccagcgcatcttctacctgaagctggaagacttggtgtctccaccttccatcattgacaagctcttcctggacaccctacctttctaatcaggagcagtggagcagtgagctgcctcctctcctagcacctgcttgctacgcagcaaagggataggtttggaaacctatcatttcctgtccttccttaagaggaaaagcagctcctgtagaaagcaaagactttcttttttttctggctcttttccttacaacctaaagccagaaaacttgcagagtattgtgttggggttgtgttttatatttaggcattgggggatggggtgggagggggttatagttcatgagggttttctaagaaattgctaacaaagcacttttggacaatgctatcccagcaggaaaaaaaaggataatataactgttttaaaactctttctggggaatccaattatagttgctttgtatttaaaaacaagaacagccaagggttgttcgccagggtaggatgtgtcttaaagattggtcccttgaaaatatgcttcctgtatcaaaggtacgtatgtggtgcaaacaaggcagaaacttccttttaatttccttcttcctttattttaacaaatggtgaaagatggaggattacctacaaatcagacatggcaaaacaataatggctgtttgcttccataaacaagtgcaattttttaaagtgctgtcttactaagtcttgtttattaactctcctttattctatatggaaataaaaaggaggcagtcatgttagcaaatgacacgttaatatccctagcagaggctgtgttcaccttccctgtcgatcccttctgaggtatggcccatccaagacttttaggccattcttgatggaaccagatccctgccctgactgtccagctatcctgaaagtggatcagattataaactggattacatgtaactgttttggttgtgttctatcaaccccaccagagttccctaaacttgcttcagttatagtaactgactggtatattcattcagaagcgccataagtcagttgagtatttgatccctagataagaacatgcaaatcagcaggaactggtcatacagggtaagcaccagggacaataaggatttttatagatataatttaatttttgttattggttaaggagacaattttggagagcaagcaaatctttttaaaaaatagtatgaatgtgaatactagaaaagatttaaaaaatagtatgagtgtgagtactaggaaggattagtgggctgcgtttcaacattccgtgttcgtactcccttttgtatgtttctactgttaatgccatattactatgagataatttgttgcatagtgtccttatttgtataaacatttgtatgcacgttatattgtaatagctttgcctgtatttattgcaagaccaccagctcctggaagctgagttacagagtaattaaatggggtgttcacagtgacttggatacaccaattagaaattaaataagcaaatatatatatatatataaatatagcaggttacatatatatatttataatgtgtctttttattaaccatttgtacaataaatgtcacttcccatgccgttattttatggttcatttgcagtgacttttaaggcagtactgtttagcactttgatattaaaattttgcttatgttttgctaaattcgaataatgtttgaagatttttaggtctaaaagtctttatattatatactctgtatcaagtcaaaatatctttggccattttgctaagaaacaaactttgaatgtcaaactgatgtcacagtagtttttgttagctttaaatcatttttgctttagtctttttaaaggaaaataacaaaactatgctgtttatattgtcattaaattatacaatcaaacaaatgccaaatgaattgcctaattgctgcaaagtataacccagataggaaatcatatgtttttttccaagagtcattctaatatttgattatgttatgtgtgcttttatgaaagattgttatttttatatatcaagatgatagaacctggaatgttaggattttgaaatgttagacttggaaggggcctggtctgtcaactagtccaaccccttaaaattcatagaggagcaaactggggcccattgaagggtgaagagttactcaaggtcaaacagctggtaacagaatcaagactaagacctaatttacctttccatactctttttttttctcaacttcatctatataaaatcaggcttttaaacataaccactaatatttacctgaagataaccatgagtaaagtatacttttgcattaattttttgagcttatatgcaaacataataaatattattaaatatcaggaaagctaacatttcatacaagatagcttcagaccaaattcaaattgaatttgaataaattagaaatactgtgcatacataaccttcttgtgcaccatgagtatttggaaagttaatccttgtttttgtcgtgtctataaaggaagaacaaaacaaaataaaaacagagccctagagaaatgctgttactttttatttttacacccatcagatttaaggaaaagactttttagccattataatctagtggttggaaggaatgaagaagcttttttagtaataggtccagatatgagtgctaaaaataaagatgatagcatgttcttctgtcttccatagttattacaactatgagagcctcccaagtcatcttatcaactcaactcccttttttttgtcttaatgttgcacataagtttatacagagtggatgaccacactagcacagaagagaacaacatgtattaaagcaggtgattcctccccttggcgggagagctctctcagtgtgaacatgccttctgtgggcggaaatcaggaagccaccagctgttaatggagagtgccttgcttttatttcagacagcagagttttccaaagtttctctgctcctctaacagcattgctctttagtgtgtgttaacctgtggtttgaaagaaatgctcttgtacattaacaatgtaaatttaaatgattaaattacattttatcaatggc

SEQ ID NO: 11 - RNA, 5676 nt, Homo sapiens
  source 1..5676: mol_type = mRNA
gcgcagccgggagagcggagtctcctgcctcccgccccccacccctccagctcctgctcctcctccgctccccatacacagacgcgctcacacccgctccctcactcgcacacacagacacaagcgcgcacacaggctccgcacacacacttcgctctcccgcgcgctcacacccctcttgccctgagcccttgccggtgcagcgcggcgccgcagctggacgcccctcccgggctcactttgcaacgctgacggtgccggcagtggccgtggaggtgggaacagcggcggcatcctcccccctggtcacagcccaagccaggacgcccgcggaacctctcggctgtgctctcccatgagtcgggatcgcagcatcccccaccagccgctcaccgcctccgggagccgctgggcttgtacaccgcagcccttccgggacagcagctgtgactcccccccagtgcagatttcgggacagctctctagaaactcgctctaaagacggaaccgccacagcactcaaagcccactgcggaagagggcagcccggcaagcccgggccctgagcctggacccttagcggtgccgggcagcactgccggcgcttcgcctcgccggacgtccgctcctcctacactctcagcctccgctggagagacccccagccccaccattcagcgcgcaagataccctccaggccctcatcaccttttttcaagtcaagatttcatcccatacatgcatgactcaatcagatttggaaatgtggatatgccctgcgtccaagcccaatatagcccttcccctccaggttccagttatgcggcgcagacatacagctcggaatacaccacggagatcatgaaccccgactacaccaagctgaccatggaccttggcagcactgagatcacggctacagccaccacgtccctgcccagcatcagtaccttcgtggagggctactcgagcaactacgaactcaagccttcctgcgtgtaccaaatgcagcggcccttgatcaaagtggaggaggggcgggcgcccagctaccatcaccatcaccaccaccaccaccaccaccaccaccatcaccagcagcagcatcagcagccatccattcctccagcctccagcccggaggacgaggtgctgcccagcacctccatgtacttcaagcagtccccaccgtccacccccaccacgccggccttccccccgcaggcgggggcgttatgggacgaggcactgccctcggcgcccggctgcatcgcacccggcccgctgctggacccgccgatgaaggcggtccccacggtggccggcgcgcgcttcccgctcttccacttcaagccctcgccgccgcatccccccgcgcccagcccggccggcggccaccacctcggctacgacccgacggccgctgccgcgctcagcctgccgctgggagccgcagccgccgcgggcagccaggccgccgcgcttgagagccacccgtacgggctgccgctggccaagagggcggccccgctggccttcccgcctctcggcctcacgccctcccctaccgcgtccagcctgctgggcgagagtcccagcctgccgtcgccgcccagcaggagctcgtcgtctggcgagggcacgtgtgccgtgtgcggggacaacgccgcctgccagcactacggcgtgcgaacctgcgagggctgcaagggctttttcaagagaacagtgcagaaaaatgcaaaatatgtttgcctggcaaataaaaactgcccagtagacaagagacgtcgaaaccgatgtcagtactgtcgatttcagaagtgtctcagtgttggaatggtaaaagaagttgtccgtacagatagtctgaaagggaggagaggtcgtctgccttccaaaccaaagagcccattacaacaggaaccttctcagccctctccaccttctcctccaatctgcatgatgaatgcccttgtccgagctttaacagactcaacacccagagatcttgattattccagatactgtcccactgaccaggctgctgcaggcacagatgctgagcatgtgcaacaattctacaacctcctgacagcctccattgatgtatccagaagctgggcagaaaagattccgggatttactgatctccccaaagaagatcagacattacttattgaatcagcctttttggagctgtttgtcctcagactttccatcaggtcaaacactgctgaagataagtttgtgttctgcaatggacttgtcctgcatcgacttcagtgccttcgtggatttggggagtggctcgactctattaaagacttttccttaaatttgcagagcctgaaccttgatatccaagccttagcctgcctgtcagcactgagcatgatcacagaaagacatgggttaaaagaaccaaagagagtcgaagagctatgcaacaagatcacaagcagtttaaaagaccaccagagtaagggacaggctctggagcccaccgagtccaaggtcctgggtgccctggtagaactgaggaagatctgcaccctgggcctccagcgcatcttctacctgaagctggaagacttggtgtctccaccttccatcattgacaagctcttcctggacaccctacctttctaatcaggagcagtggagcagtgagctgcctcctctcctagcacctgcttgctacgcagcaaagggataggtttggaaacctatcatttcctgtccttccttaagaggaaaagcagctcctgtagaaagcaaagactttcttttttttctggctcttttccttacaacctaaagccagaaaacttgcagagtattgtgttggggttgtgttttatatttaggcattgggggatggggtgggagggggttatagttcatgagggttttctaagaaattgctaacaaagcacttttggacaatgctatcccagcaggaaaaaaaaggataatataactgttttaaaactctttctggggaatccaattatagttgctttgtatttaaaaacaagaacagccaagggttgttcgccagggtaggatgtgtcttaaagattggtcccttgaaaatatgcttcctgtatcaaaggtacgtatgtggtgcaaacaaggcagaaacttccttttaatttccttcttcctttattttaacaaatggtgaaagatggaggattacctacaaatcagacatggcaaaacaataatggctgtttgcttccataaacaagtgcaattttttaaagtgctgtcttactaagtcttgtttattaactctcctttattctatatggaaataaaaaggaggcagtcatgttagcaaatgacacgttaatatccctagcagaggctgtgttcaccttccctgtcgatcccttctgaggtatggcccatccaagacttttaggccattcttgatggaaccagatccctgccctgactgtccagctatcctgaaagtggatcagattataaactggattacatgtaactgttttggttgtgttctatcaaccccaccagagttccctaaacttgcttcagttatagtaactgactggtatattcattcagaagcgccataagtcagttgagtatttgatccctagataagaacatgcaaatcagcaggaactggtcatacagggtaagcaccagggacaataaggatttttatagatataatttaatttttgttattggttaaggagacaattttggagagcaagcaaatctttttaaaaaatagtatgaatgtgaatactagaaaagatttaaaaaatagtatgagtgtgagtactaggaaggattagtgggctgcgtttcaacattccgtgttcgtactcccttttgtatgtttctactgttaatgccatattactatgagataatttgttgcatagtgtccttatttgtataaacatttgtatgcacgttatattgtaatagctttgcctgtatttattgcaagaccaccagctcctggaagctgagttacagagtaattaaatggggtgttcacagtgacttggatacaccaattagaaattaaataagcaaatatatatatatatataaatatagcaggttacatatatatatttataatgtgtctttttattaaccatttgtacaataaatgtcacttcccatgccgttattttatggttcatttgcagtgacttttaaggcagtactgtttagcactttgatattaaaattttgcttatgttttgctaaattcgaataatgtttgaagatttttaggtctaaaagtctttatattatatactctgtatcaagtcaaaatatctttggccattttgctaagaaacaaactttgaatgtcaaactgatgtcacagtagtttttgttagctttaaatcatttttgctttagtctttttaaaggaaaataacaaaactatgctgtttatattgtcattaaattatacaatcaaacaaatgccaaatgaattgcctaattgctgcaaagtataacccagataggaaatcatatgtttttttccaagagtcattctaatatttgattatgttatgtgtgcttttatgaaagattgttatttttatatatcaagatgatagaacctggaatgttaggattttgaaatgttagacttggaaggggcctggtctgtcaactagtccaaccccttaaaattcatagaggagcaaactggggcccattgaagggtgaagagttactcaaggtcaaacagctggtaacagaatcaagactaagacctaatttacctttccatactctttttttttctcaacttcatctatataaaatcaggcttttaaacataaccactaatatttacctgaagataaccatgagtaaagtatacttttgcattaattttttgagcttatatgcaaacataataaatattattaaatatcaggaaagctaacatttcatacaagatagcttcagaccaaattcaaattgaatttgaataaattagaaatactgtgcatacataaccttcttgtgcaccatgagtatttggaaagttaatccttgtttttgtcgtgtctataaaggaagaacaaaacaaaataaaaacagagccctagagaaatgctgttactttttatttttacacccatcagatttaaggaaaagactttttagccattataatctagtggttggaaggaatgaagaagcttttttagtaataggtccagatatgagtgctaaaaataaagatgatagcatgttcttctgtcttccatagttattacaactatgagagcctcccaagtcatcttatcaactcaactcccttttttttgtcttaatgttgcacataagtttatacagagtggatgaccacactagcacagaagagaacaacatgtattaaagcaggtgattcctccccttggcgggagagctctctcagtgtgaacatgccttctgtgggcggaaatcaggaagccaccagctgttaatggagagtgccttgcttttatttcagacagcagagttttccaaagtttctctgctcctctaacagcattgctctttagtgtgtgttaacctgtggtttgaaagaaatgctcttgtacattaacaatgtaaatttaaatgattaaattacattttatcaatggc

SEQ ID NO: 12 - RNA, 2800 nt, Homo sapiens
  source 1..2800: mol_type = mRNA
gcgcagccgggagagcggagtctcctgcctcccgccccccacccctccagctcctgctcctcctccgctccccatacacagacgcgctcacacccgctccctcactcgcacacacagacacaagcgcgcacacaggctccgcacacacacttcgctctcccgcgcgctcacacccctcttgccctgagcccttgccggtgcagcgcggcgccgcagctggacgcccctcccgggctcactttgcaacgctgacggtgccggcagtggccgtggaggtgggaacagcggcggcatcctcccccctggtcacagcccaagccaggacgcccgcggaacctctcggctgtgctctcccatgagtcgggatcgcagcatcccccaccagccgctcaccgcctccgggagccgctgggcttgtacaccgcagcccttccgggacagcagctgtgactcccccccagtgcagatttcgggacagctctctagaaactcgctctaaagacggaaccgccacagcactcaaagcccactgcggaagagggcagcccggcaagcccgggccctgagcctggacccttagcggtgccgggcagcactgccggcgcttcgcctcgccggacgtccgctcctcctacactctcagcctccgctggagagacccccagccccaccattcagcgcgcaagataccctccagatatgccctgcgtccaagcccaatatagcccttcccctccaggttccagttatgcggcgcagacatacagctcggaatacaccacggagatcatgaaccccgactacaccaagctgaccatggaccttggcagcactgagatcacggctacagccaccacgtccctgcccagcatcagtaccttcgtggagggctactcgagcaactacgaactcaagccttcctgcgtgtaccaaatgcagcggcccttgatcaaagtggaggaggggcgggcgcccagctaccatcaccatcaccaccaccaccaccaccaccaccaccatcaccagcagcagcatcagcagccatccattcctccagcctccagcccggaggacgaggtgctgcccagcacctccatgtacttcaagcagtccccaccgtccacccccaccacgccggccttccccccgcaggcgggggcgttatgggacgaggcactgccctcggcgcccggctgcatcgcacccggcccgctgctggacccgccgatgaaggcggtccccacggtggccggcgcgcgcttcccgctcttccacttcaagccctcgccgccgcatccccccgcgcccagcccggccggcggccaccacctcggctacgacccgacggccgctgccgcgctcagcctgccgctgggagccgcagccgccgcgggcagccaggccgccgcgcttgagagccacccgtacgggctgccgctggccaagagggcggccccgctggccttcccgcctctcggcctcacgccctcccctaccgcgtccagcctgctgggcgagagtcccagcctgccgtcgccgcccagcaggagctcgtcgtctggcgagggcacgtgtgccgtgtgcggggacaacgccgcctgccagcactacggcgtgcgaacctgcgagggctgcaagggctttttcaagagaacagtgcagaaaaatgcaaaatatgtttgcctggcaaataaaaactgcccagtagacaagagacgtcgaaaccgatgtcagtactgtcgatttcagaagtgtctcagtgttggaatggtaaaagaagttgtccgtacagatagtctgaaagggaggagaggtcgtctgccttccaaaccaaagagcccattacaacaggaaccttctcagccctctccaccttctcctccaatctgcatgatgaatgcccttgtccgagctttaacagactcaacacccagagatcttgattattccagagtaagttttatgatttcctgctttcaaatgaatgatcagggtctctatttatggctactagtaataagagttgattgaatgattttgtgtctggcaccatgttagacagttttcatactttttctatatttctcgcttcatttagcaattcagtgcatccattgcagcaaataatttttgccttattgaatctctaaatgccttaacaagtgaccctgacagtgctgcacctgtcatacacattgttgcaggattcctggtggttgtgccaatgaaaatctgcacagacaaactacaatttgtagatttatctcgtgatctagacaaagtgactactgttttttttcatattgtgttcaaaccatctgggtgagcctcaagttattactaagcagtttatccaattgcatcagcattgattgacctgctgcttattcagagggattagaccattggtggagagaagcgagctctggctgtgacatcagttgtcaggagaactgggatggaacgccacctctgctacttcctgcctgtgtcacctggttcctgcttttggctctctcctgcctacagtggaaatggctacaatatttaacctcagaggggtgttctgaacaccagtcacacaagaatgatggttgtgaaaatgtttttaagaccataaatgacgattggtgttgttatagttaagtcaaccaaaacatatcaacatttttatttcatatgggatatagtaaaatcccatcagtctgaaccctgcaaattcctcaaaatttggataacacagagaagaaccatgagaaaggatttcctgagcaaatgaataaacagctctgtaaggcaa

SEQ ID NO: 13 - protein, 611 aa, Homo sapiens
MWLAKACWSIQSEMPCIQAQYGTPAPSPGPRDHLASDPLTPEFIKPTMDLASPEAAPAAPTALPSFSTFMDGYTGEFDTFLYQLPGTVQPCSSASSSASSTSSSSATSPASASFKFEDFQVYGCYPGPLSGPVDEALSSSGSDYYGSPCSAPSPSTPSFQPPQLSPWDGSFGHFSPSQTYEGLRAWTEQLPKASGPPQPPAFFSFSPPTGPSPSLAQSPLKLFPSQATHQLGEGESYSMPTAFPGLAPTSPHLEGSGILDTPVTSTKARSGAPGGSEGRCAVCGDNASCQHYGVRTCEGCKGFFKRTVQKNAKYICLANKDCPVDKRRRNRCQFCRFQKCLAVGMVKEVVRTDSLKGRRGRLPSKPKQPPDASPANLLTSLVRAHLDSGPSTAKLDYSKFQELVLPHFGKEDAGDVQQFYDLLSGSLEVIRKWAEKIPGFAELSPADQDLLLESAFLELFILRLAYRSKPGEGKLIFCSGLVLHRLQCARGFGDWIDSILAFSRSLHSLLVDVPAFACLSALVLITDRHGLQEPRRVEELQNRIASCLKEHVAAVAGEPQPASCLSRLLGKLPELRTLCTQGLQRIFYLKLEDLVPPPPIIDKIFMDTLPF

SEQ ID NO: 14 - protein, 637 aa, Homo sapiens
MHDSIRFGNVDMPCVQAQYSPSPPGSSYAAQTYSSEYTTEIMNPDYTKLTMDLGSTEITATATTSLPSISTFVEGYSSNYELKPSCVYQMQRPLIKVEEGRAPSYHHHHHHHHHHHHHHQQQHQQPSIPPASSPEDEVLPSTSMYFKQSPPSTPTTPAFPPQAGALWDEALPSAPGCIAPGPLLDPPMKAVPTVAGARFPLFHFKPSPPHPPAPSPAGGHHLGYDPTAAAALSLPLGAAAAAGSQAAALESHPYGLPLAKRAAPLAFPPLGLTPSPTASSLLGESPSLPSPPSRSSSSGEGTCAVCGDNAACQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPICMMNALVRALTDSTPRDLDYSRYCPTDQAAAGTDAEHVQQFYNLLTASIDVSRSWAEKIPGFTDLPKEDQTLLIESAFLELFVLRLSIRSNTAEDKFVFCNGLVLHRLQCLRGFGEWLDSIKDFSLNLQSLNLDIQALACLSALSMITERHGLKEPKRVEELCNKITSSLKDHQSKGQALEPTESKVLGALVELRKICTLGLQRIFYLKLEDLVSPPSIIDKLFLDTLPF

SEQ ID NO: 15 - protein, 598 aa, Homo sapiens
MPCIQAQYGTPAPSPGPRDHLASDPLTPEFIKPTMDLASPEAAPAAPTALPSFSTFMDGYTGEFDTFLYQLPGTVQPCSSASSSASSTSSSSATSPASASFKFEDFQVYGCYPGPLSGPVDEALSSSGSDYYGSPCSAPSPSTPSFQPPQLSPWDGSFGHFSPSQTYEGLRAWTEQLPKASGPPQPPAFFSFSPPTGPSPSLAQSPLKLFPSQATHQLGEGESYSMPTAFPGLAPTSPHLEGSGILDTPVTSTKARSGAPGGSEGRCAVCGDNASCQHYGVRTCEGCKGFFKRTVQKNAKYICLANKDCPVDKRRRNRCQFCRFQKCLAVGMVKEVVRTDSLKGRRGRLPSKPKQPPDASPANLLTSLVRAHLDSGPSTAKLDYSKFQELVLPHFGKEDAGDVQQFYDLLSGSLEVIRKWAEKIPGFAELSPADQDLLLESAFLELFILRLAYRSKPGEGKLIFCSGLVLHRLQCARGFGDWIDSILAFSRSLHSLLVDVPAFACLSALVLITDRHGLQEPRRVEELQNRIASCLKEHVAAVAGEPQPASCLSRLLGKLPELRTLCTQGLQRIFYLKLEDLVPPPPIIDKIFMDTLPF

SEQ ID NO: 16 - protein, 598 aa, Homo sapiens; identical to SEQ ID NO: 15

SEQ ID NO: 17 - protein, 652 aa, Homo sapiens
MTSAQYKIKILIEGLHHGQRPGPAPPRQPGSFCWALKADGIMWLAKACWSIQSEMPCIQAQYGTPAPSPGPRDHLASDPLTPEFIKPTMDLASPEAAPAAPTALPSFSTFMDGYTGEFDTFLYQLPGTVQPCSSASSSASSTSSSSATSPASASFKFEDFQVYGCYPGPLSGPVDEALSSSGSDYYGSPCSAPSPSTPSFQPPQLSPWDGSFGHFSPSQTYEGLRAWTEQLPKASGPPQPPAFFSFSPPTGPSPSLAQSPLKLFPSQATHQLGEGESYSMPTAFPGLAPTSPHLEGSGILDTPVTSTKARSGAPGGSEGRCAVCGDNASCQHYGVRTCEGCKGFFKRTVQKNAKYICLANKDCPVDKRRRNRCQFCRFQKCLAVGMVKEVVRTDSLKGRRGRLPSKPKQPPDASPANLLTSLVRAHLDSGPSTAKLDYSKFQELVLPHFGKEDAGDVQQFYDLLSGSLEVIRKWAEKIPGFAELSPADQDLLLESAFLELFILRLAYRSKPGEGKLIFCSGLVLHRLQCARGFGDWIDSILAFSRSLHSLLVDVPAFACLSALVLITDRHGLQEPRRVEELQNRIASCLKEHVAAVAGEPQPASCLSRLLGKLPELRTLCTQGLQRIFYLKLEDLVPPPPIIDKIFMDTLPF

SEQ ID NO: 18 - protein, 598 aa, Homo sapiens
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPTTPGFQVQHSPMWDDPGSLHNFHQNYVATTHMIEQRKTPVSRLSLFSFKQSPPGTPVSSCQMRFDGPLHVPMNPEPAGSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQEPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 19 - protein, 535 aa, Homo sapiens
MDNYSTGYDVKPPCLYQMPLSGQQSSIKVEDIQMHNYQQHSHLPPQSEEMMPHSGSVYYKPSSPPTPTTPGFQVQHSPMWDDPGSLHNFHQNYVATTHMIEQRKTPVSRLSLFSFKQSPPGTPVSSCQMRFDGPLHVPMNPEPAGSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQEPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 20 - protein, 626 aa, Homo sapiens
MPCVQAQYSPSPPGSSYAAQTYSSEYTTEIMNPDYTKLTMDLGSTEITATATTSLPSISTFVEGYSSNYELKPSCVYQMQRPLIKVEEGRAPSYHHHHHHHHHHHHHHQQQHQQPSIPPASSPEDEVLPSTSMYFKQSPPSTPTTPAFPPQAGALWDEALPSAPGCIAPGPLLDPPMKAVPTVAGARFPLFHFKPSPPHPPAPSPAGGHHLGYDPTAAAALSLPLGAAAAAGSQAAALESHPYGLPLAKRAAPLAFPPLGLTPSPTASSLLGESPSLPSPPSRSSSSGEGTCAVCGDNAACQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPICMMNALVRALTDSTPRDLDYSRYCPTDQAAAGTDAEHVQQFYNLLTASIDVSRSWAEKIPGFTDLPKEDQTLLIESAFLELFVLRLSIRSNTAEDKFVFCNGLVLHRLQCLRGFGEWLDSIKDFSLNLQSLNLDIQALACLSALSMITERHGLKEPKRVEELCNKITSSLKDHQSKGQALEPTESKVLGALVELRKICTLGLQRIFYLKLEDLVSPPSIIDKLFLDTLPF

SEQ ID NO: 21 - protein, 637 aa, Homo sapiens; identical to SEQ ID NO: 14

SEQ ID NO: 22 - protein, 443 aa, Homo sapiens
MPCVQAQYSPSPPGSSYAAQTYSSEYTTEIMNPDYTKLTMDLGSTEITATATTSLPSISTFVEGYSSNYELKPSCVYQMQRPLIKVEEGRAPSYHHHHHHHHHHHHHHQQQHQQPSIPPASSPEDEVLPSTSMYFKQSPPSTPTTPAFPPQAGALWDEALPSAPGCIAPGPLLDPPMKAVPTVAGARFPLFHFKPSPPHPPAPSPAGGHHLGYDPTAAAALSLPLGAAAAAGSQAAALESHPYGLPLAKRAAPLAFPPLGLTPSPTASSLLGESPSLPSPPSRSSSSGEGTCAVCGDNAACQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPICMMNALVRALTDSTPRDLDYSRVSFMISCFQMNDQGLYLWLLVIRVD

SEQ ID NO: 23 - DNA, 81258 bp, Homo sapiens
  source 1..81258: mol_type = genomic DNA
gcacctcgcgagactccacccagccccttccccgaacagcgacccgtccccgttcctagctgaggtcggtgctaggcggaggacgcgagagggagagcggaagggggccgccgggcacgccgggaagtgtggtccacgcgcttcacgcggccatattcttccggagcgggggaggccagagaggccgttgggactacgactcccaggcgccctttcggtttgtttttgttcaaaaaaaaaaaaccccacactccccctcctcactcttcacacacactcacacacacccacggcagacacgcacgcacccgggcgccgaagggaaagccgcgtctcgccctcccgccccgccgtcggtcctgtctcagtccctcagcagagcgggaaagcggaggccggagccgtgacctctgaccccgtggttatgcggagccgccgcattccttagcgatcgcggggcagccgccgctgccgccgtgggcgactgacgcagcgcgggcgcgtggagccgccgccgcccctcccccaccgccgctctcgcgccagccggtccccgcgtgcccgccccttctccccggccgcacccgagacctcgcgcgccgccgctgccacgcgccccccccaccgccgccgccgccccagccccgcgccaccgccccagcccgcccagcccggaggtcccgcgtggagctgccgccgccgccggggagaaggatgaaggacaaacagaagaagaagaaggagcgcacgtgggccgaggccgcgcgcctggtgaggcggacagccgaggggggctccgtggggcccggggtggggggggctcgccgcgcacccccccactggggggggaggggcaaggccctgcccaccgcgggagggggcggggccatcttcctttaagaacgggacagccccgcaaggcgaggggtggggagcgctccgccgggatgggatgtggcgcctttttccgctcgccctcgcgccccccccgccccgcgcagctaaattccggcggaggggcgagctggcaggccggctcctcccactctgggcagcggggtcccgcgtcccctcccccactatttggcagcgtctgggggtctggggcagcttcgttcattcacccgggggagttgggtttccgggaagggtcggaagctcctccctcgcttcctggtgggtaatggggtggtgcctttgactccgggggtggaaaagcgaccccacattcaaggacgccaatggcatgttgagctttcccaatctaaaccaggtgcgtggagggaagcaagtgcttactcccagcttgaaccctgagcagcggttctctaactttagtgtcctgtgggcgacacctgggaggcggttaggaacgctactcctagggcccttcgtaagacctgctgagcctcagggtctgggatagagcctgggaatctgcgctttcaccagcttcccaggtgattcaaacgcagtggctcacggaccccactttgagaaacgcagactgggctgtttgccttcagaagctggactggaaattggtattttgaggagaactgaatccactttgcaaagaatgtatgttggacaaagacaaagtatctctgttgggagacgttgttcactgaggatttagcaagggagagcgtaacttgagtgtttgaagccgtctcgttcaaccgatgggggttgtgaattttgtgttcagagcgtcagaggacttgcaggtgaaatagcttccttttggattactcccacaaagctcagacactttgagacatattcgggtaattatctgctgataccagtagtaacaggttatactaagatgtcagttcctaagatctgtcacacagcgaggttgtggtgcggatagcataagtgtctcttgacactgtagtttatatgtaatggttgtaattcagttttaatgatgagtgaagctcagacttaattttctctcttattttgaagtgagttttctgcttcaaaggtgttgtgcaaaaaaaaaaaaaatagactgtattgggggggttgacacaaacctgaagtacacctttttctttctcgaagtctgtaaaagttacagatttataaaaactgaaggaatttgggctacagaattttcattggtgcttgctatcctcttccctagtcatttgttgcccagggaacaaaaatagcaattgtttgtatgctgaagttttgtgcttcagtttattttggcctggcatgtgaagtttgaagattttgaggttccgaaatattttttttctgattgagtcaaaattggaagcttgggctagttttcaggatcttgtagaaatttcaggatttctctaacggttctgcacctggaaacaaatataggcctgatggggagtgggagagagctctcgttctcctcctcgacaatgagttttgtgtgttcattgacattctccttggcgttcctgttatgcgggtgtttatgaccctgggcacccaaggaaatttgcttgccagagttttacttgttagaggtaggtgaaagtacttttaagtcttgtgttatttatgtcttgacagaattatgaaatctgtttgtctcaaagttctgttggctcttactagttgactttaggaatttggggtccactgttaagtctctttagattctcgaatttaccgtggtctgaacttcaacatcgaggttgctatttctgttttatcattgactcaacttcaagccaggttatcagggtaagttattcctctttttcttctaaggctcttcattaaaaatagtaatatgaataggaattactttgtttgtttgtttttcgtttgtttgccaccacacccggctaattttttatttttagtaaagatggggtttttccatgctggtcaggctggtctcgaactcgtgaccttaggttatccgcccaccttggcctcccaaagtgctgggattacaggcgtgagccaccgcgcccagcctatgagtaggaattactttgtggtgctgttatacatgtgaaaaaggatttgcgagactttgggagtctgaggtggaataatcacttgaggctaggaattcgagatgagcctgggcaacatagacccggtctctacaacaacagcaacaaaatgagctgggcatggtggtacacacctgtggtcctagctactcaggaggctgaggatagcttaggccgaggagttggaggctgcagtgcccaatgattgattgtaccactgcactccagcctgggtgatagagggagaccctgactctaaaaaaataatgaggctgggcgctgtggctcacgcctgtaatcccagcactttgggacgccgaggtgggtggatcacagggtcaggagttctagaccagcttggccaacaaggtaaaacccagtctctactaaaaatacaaaaattagccgggtgtggtggtgcacgcctgtaatcccagctactgagggggctgagacaggagaatcacttgaacttgggaggtggaggttgcagagagccaaggccatgccactgtccagcctgggtgacagagcaagactcgtctcaaaaaaaaaaaaaaaagaaaaaaagaaaaaggacttgttgagaatgtgatatgcaatttgagtatcttgtgtgtgttttttgttttagaattttccatttctttgaatgggactggatggtaggtgagggggaactttttaagagtcccaggccgggcacggtggctcacgcctgtaatcccagctctttgggaggccgaggcagacggatcacctgaggccagaagttcgagaccagcctggccaacatggtgaaaccccgtctctactaaaaatacaaaaattagccaggcatggtgatggtgcatgcctgtaacctcagctactcgggaggctgaggcaggagaatcacttgaactcgggaggtggaggttgcagtgaacccagatcacaccactgcactccagcggcgacagtgagactctgtctcaaaaacaaaacaaaagaaaaaaccagtcctcttaatgtttaaaaaaagttgctgtaacactgtactggagaatttgtgagagcgtacactggactgagagaaacttgactaggagggaacgtaggacagttgattagaacacctgaacacttctctttgggcaggtgaatttggagtgcttgcaatggcaattgtatgcacttccatgggaacgctttcagattgctcctgaccccactcagattggtgactagagtgttactgggaatccttttaaaaattgtttttgtaggctgtgcgcggtggctcacgcctgtaatcccagcactttgggaggccgaggcgggcggatcacgaggtcaggagatcgagaccatcctggctaacacggtgaaaccccatctctactaaaaatacaaaaaattagccaggcgtggtggcgagtgcctgtagtcccagctactcgggaggctgaggccggagaatggcgtgaacgcgggaggcggagcttgcagtgagccgagatcgagctactgcactccatccagcctgggcgacagagcaagactctgtctcaaaaacaaacaaacaaaacaacaacaacaaaaaaacctttcgtagaggaaagaagattcttaagaatttcagtatagtgtaccccaggagattgatttttgcagatgttaattatgctgttgaaaaatagtctttctttcacacattttgtagtattgtcttttctaggtgtaagaagtagaccttatgtggttgagtggatataaatcctccaacttgttataaacagtttgtttgggagaaatagccaaggggatttccctttaaatcgaggacgctaaatggattctccctggcttcgagaaactctttgttgtatgctttatcttttactttgattcatgactgaccagttcttacatcaaacatttttttcccctttgaagtaagagaagagatacttaaaaaaaaaaatgtagtctagaaagagactaggaatagaacactgctgacttgttcttataccaagagctggaggcataaacagttttaataaaaaagaatactttgtatcttttgatgtaacttccctttagagattctgtttcccctgtcttaaattctcaggctatttctgattgctgtttaaataattggtagttactatttgagtcatgcttcttaaaaaaaaaagatataaaaattcgccagctcagctttcatttaattagtatcaaagaaatgttatctgtggcagtgaacattagtagcttgttccagggtttattgtaggtttgttcaagtcagtactgcccatcttggcacttaggttcttggaatacagaggtggataaggtttgcccctcagggcttatggtcttctggggatgaagaaggtccagacgcaggaacagaattttactacagtcatgatacgtgttatcatagttgtataaatcataaacagatatcccagttgctgcctcttgtctagtcatagtttctcctttcactctcaactgtcctggtttggaagataattgtatgtgttatgaaacaatgggaacattgaaaagagacatttacctctgcctggcagagatcagggaaggcttcctggaggaaggtgctttctgagctgaatcttgaagggtaactaggagttaggccaatcacagagggcataagcaggagctggtgacaacacatagcaggagagaaagagtcagagtgggtgagagggagtgtgtgtatttgcaacgtgtgggaattaggtatatctacaaattattggaacataaagtttgtagagtgtcactgtggatgggtagagaggcagagaccagattgtgagggttccatatgccaggccagtgagcacctactctgtgccagacattgtaggcattacttacattgtctctaatctgtccaacaactcgagaggaagacatcagtatccttcaaaagagaacctaggttcaggttgggttgtaattgccccagtttagaatgtaaaaatttgaatctaactctagatcctgttctttaccggtgggccagtggactttggaagcttcagcttttgcagtatgtgattaaggagctgttgaaggattttatttgctttctattattattattactattttttgagacagggtctcactctgtcaccctggctgaagtgcagtggtgccatcacagctcactgcagcttccacttcctgggctcagatgattctcccacctcagcctcctgtgtagctgggattacgggcatgagccaccatgcccatctagttttttgtatttttttgtagagacagggtttcaccatgtcgcccaggctggttttgaactcctaggctcaagcaatctgcccaccttggcctcccaaagtgctgggattacaggtgtgacccactttacccagccatatatattttttaaattctttaaagagatgaggtttcactgtgttacccaggctggagtgcagtggctatacacaggcacgatcatagtgcactagagcctggagctcctgagcgcagcaagtgaccctcctgcctcagtctcagctgagactataggtgtgcaccaccgtgcctggcctgttgaaggattttagagctgaaagtgatgagggcaggtttgctttgctgggttgacctcagtctataacatgtaagttctaccagagcagggatttggtcttgtttactgtgaatccctagaattgtgtctggcacaaggtacgtgttccatgtgtttgttgaatgaaggatgacaacagagtcagaatggatttgaggaaagcaggactggatggagggataaatattaagaggctgttgtgttgggtcaggtaagagactggttttgggacttggcggacagatgagcgagaaagatgacacctgcgtttctgaattaggtgattatacaggctgtcaatcaaggtagggaatacaggaagaagagtgggtctggagaagaggtgaattagagttgccaattagatgaattaagagtagagctgaattagagtagataatgaaggggtatagtcagtacacaactgaatatttgaatctgaaatttgggggagaagtcagagctagaggtagagattaggtagttatcagaatttaggtagtagctgaaactgagatgataatgttacccatggagaatggaaaatctgaaaagagaagcaaagaataggaccatgggaaataggggttttattttagtaccttctccaaaccttgactttggtgctgaggtcttatgttcttccatagagagtaccttccgcagcagacctatcagacgagatcgggcacattcagggtggtatggctgtagacagcagcatcgtatcagacttctttatgttttgcttgtgacagcagtacttggctatggcagttaaatggactgtaagatgttagcttgatttggagtaccctgagtagctgcacaggtagacaaagtacaaagatattgacagcttttttcttcctttagaagaataacatatctaaaacaactcttaaagcaagagagctgcagttgcacactaagaaaatactgcaatactgccatgacttggttataggtgatgctaacataaatgggatattctgtttctcattgcaccagtcatgcagaatcatagcatcatgtgccagatggcagaacgtactgtggcagttaacaccacaaattaacaacacagtggcagaaagcaccgtttaaaaatatgggtacttggccaggcgtggtggctcacttctataatcccagcactttaggaggctgaggcgggcagatcacctgaggtcaggagttcgagactagctctagtctctactaaaatacaaaaattagtgtgtgtgtgtatatatatatactaatcaaacttccttgggaaagaaaatgtgaatgcatttctgtgtcatacatttcttgtgcagcactatatattctgtggtttagcagtattaacagcatcaagctgaacagtaaccagtgttgctaaggtctcgcatactataccatgtatgctgagaagatatgcatccttagaattgagaattagaacatttcagttaattgcagtaacttttcagtaactagatatatgtgtaatttgatttctgaagtaattttaactgtggaaaaactaagttgtcaccagcggtacctcatagcataactttagcccatgatatatggatggatggatataaatggaaattgcacactgaaattaggacgtttatatttcttcaggtattagaaaactactcggatgctccaatgacaccaaaacagattctgcaggtcatagaggcagaaggactaaaggaaatgaggtttgtattgttcttgttgcttaacatgagggtttcataggatatgtgtctttctgtagttgtagtgatatgagtaagtacacctgtgtatgtatttgtgcttctgtgtctggtagtgagatggaactatccatttcccaaattgtgtgtcgtctcagtggagggtggcagtgggtgaacatccatttgttttcatttagaggtgcagtggctttccccatctccttgttcttccttaaatcttggcatagagcttgttttttgtgtaaggagtcagggcacattttctcttgtgctgttttggaagtttcagacctgatagcctctattgtgcctaacgctacccttttctctatgccgtatcactgaagttctttcttttagaataaaagaacagtggtctcatcaacataaaaaaccactgacttttttccccttactaattgcagtcatatatgatgtgaatttactaaattttgaggtgacctttattttccaaatttatggtgtagttttcctggagaagtaacaagttagaaacatagaatttgcaggcttgaaagccaggactatctttcatctttctttttcctcatcctaacttcaaacttaaaaaaaaaaaaaaaaaaagctgggggtagtggcccatgcctgtaatcccagcactttgggaggccaaggcaggcagatcacttaggtcaggagttcgagaccagcctggccaacatggcaaaaccccgtctctactaacaatacaaagattagccggcatggtgacgcatgcctgtagcctgtagttccagcttgggaggcggaggctggagaatcgcttgaactcaggagacggaggttgcagtgagccgagattgcaccactgcactccagcctggacaacagaataagactgcatctcaaaaaacaaaacaaaacaaaaaacaaagaaagtacttaaatgaggaaagaggagagcactacacagtccttgcctgggactcagagcaaacttcatgtttttgctagtcaaacttagctgctttaattgaccttttagggtcaaaaggccttcactttccagttgtcagttcaaacgttctcaagttgatgttttgtggggagggaagattgagattgtctacatcacatttatttgacttgtacttatccactgtgtagtgttataattgtgctttatttttgtttttatgggctatttcccatcatgctgctgtctgcactgctgttggaccactttgcctatagaaggtaaatgagtccctttgggacatatggaattgagaaccttaacttgtttctgcttcttgttcttagaggaattatgaagcagtcatgatggctcatgatgagtgagcagctctgtaatttgtagacagaggtataatatgtaggagtcacagaagagctgtgccagcatgctgttccccttgtagcagagatgctagcacacccacttgatactccagattttgcatagggtttcttttagagattggttcttggtgaaaaattttttagcatccagtgtgttccaaggcaaaataagtaatagttttttaatggtttttttagactcagaatttatttttaacactgcataaattagggcttctagttctgaaaaatgcaaagaggcttacttaagctatttatatattttttaacatgctggggagagggaagagagggtgcacaggtgactcctagcactaatatgctagtgccaaactcagttaatattaataatggtcatttgttgatatttttggtagcttttctatttaaagattcagtaagtagcatcctttgcactgaattgtcacgttttgtaacctgctgttgtggcagggaccatgaaacaacttagtatgccattgtttcagtttattttttaagcattactgggaactaaatccacttgacttactttttgcatttaggtttaaataattgttcctgggtagatacagaataaaactcaatctccccacttccagcagagaaaaaacaaatgaaatactgttctttttgtgtacttactagaaaatgtttaactggaaaaaaagttctgcttgttttattggcacagcaccattgattctgtgattctttgtcatagtattctgtggcagtttctgagtatgatatgtagattgaaatatagaggctaggaaacttttaatagtaaaattttactaaaaatttattaacttattaaaatataacttgaaaaaattgtgaggtttttctaaatttcttggaaatttgatttatattgtcaaaaagcactgaagaagtgttagttatggatttcgggtatcacataatgtttattccagagttttctgagtgtgatttatgtgaatttcatttggttattcttttactattttcacagtgaaattgtcatgagattttttcttctgtatttcttgcttagcttcttctcatttaagaatgagttggagggattaggtctttaagtaggcagatggattgtataaccctcatccattcttttgtggttttacagtgggacttcccctctcgcatgcctcaatgctatgctacattccaattcaagaggaggagaggggttgttttataaactgcctggccgaatcagccttttcacgctcaaggtaagtgatatgaactctcttttggtgcagtgattcttggacttttaatgtgcataaaaatcacctggtaggtctggaataggggccatgaatttgcatttctcatatgcagtcaggtgacactgatgtttttcttttttttagacatagtctcgctgtgtcacccaggctggagtgcaatggggcgagatctcggctcactgcaatctctgcctcccatgttcaagtgattctcccacctcaacctcccaagtagctgggactacaggcacatgccaccatgctcggctaatttttttattttttttaatagagacggggtttcaccatgttggccaggctggtctcgaaatcctgacctcaggtgatccacctacctcggcctcccaaagtgctgggattacaggcatgagccaccgcacctggccagatgctgttggttctaaatggcactttgaggtgttctaatatgaatcagatattagaaattatttagtcatagcagctcttgtttgtgtgttcatcttttcttgtctatatctttgtagattgtacaaaactgaagcataactatggggataattgcagatgtgcctttttttttttttttttttttttattctgagatggagtctcactctgttgcccaggctggagtgcagtggtgcgatctcagctcattgcaccctccacctcccaggttcaagcaattcttctgcctcagcctcctgagtagctaagagtgcactaccatgcccaggtaatttttgtatttttagtagagacggggtttcaccatgttggccaggctcgtcttgaactcctgaccttgtgatccgtccgccttagcctcccaaagtgctgggattacaggcgtgagccactgcacctggccacagatgtgccttaattaggattttgttgtagtaatgatgtgaaggataaattactggtcagctaatgtaagacctactctatggaaaataactgaaatgtgaacatatttgtggaaattaagtgacagtaattataaagtaagagtacttaaatcaggtagagttcataataaagcttaaatagggccgaaatgaacaggctaattatttttccaccatttgtgatatatatggtaatcatttagaccgggggtctgcaaagtttttctgtaaaggaccagaaaatcaatgtttttttgttttgtggatcatactgtctttgtcttaaccactcaggtctgccattgtagtaggaaagcagccatacccaaatgaatgggcatggctgtgttccaaaaaaaaaaaaaatttatttacaaatgtttgttttacttacgaaaacaggctgcaggccagatttgatttggggctgtactttgtcgacccctgatttagacataagatgacttgtcgacattggagaaactgaaaatttgatgctgaccctttcgttcttccatagtgggttataaattttagcagtcagtacaaactttttcctttaagttttgttttcttgatggactgtattcaaaataaatacctgaacaaataccaactatataacatgaagccataaaacttggtctttatatataaataaagtttatcttggggtcagatgtctgtgctgttttctatatttttttatatccaatagtttggtctttttcctttcagttttgtatgcttttcttgtaatcgagccttggaagagacagagccactgagcccaaaacctagctttgtggtcggctgtggtggctcatgcctgtaatcccagcactttgggaggctaaggcgggaggattgcttaaactcaggagtatgagaccttgtctctattaaaaaaaaaaaaaaaaaaaaaaagccttgcgtggtggtgtgtgtgcttatagtcttagctactcaggaggctgaagcaggagggtagcttggagatggaggctgcagtgagctgtgatcacaccactgctctccagcctgggccacatagcaagaccctgtctcaaaacaaaaacttagctttgtaacctgggagggatttaacccttctttttggtggaatatgcatccctggcattgagtcagtccttaataaatgatagcctttatgatgatctgaggtttatcattcttaattgcctgttaatggcatttttctattaatattatctattttttgagatagggtcttgttctgtcacctgggctggagtgcagtatcatagtcacagctcactgcagccttgacctactaggctcaagtgatcctcccacctcagccttctgagtagctaggacttcaggcgcataccactatgcctggctaattttttgtattttttgtggagaaggagttttgccatgttgcccaggctggtctcgagctcctggggtcaagcgatccactggctttggcctcccaaactgccaggattacagacgtgggccactgcacctggcctattattttttaatttaatattaatttatatttttagaaacagggtctcacactgttgtccacagctggagtgcagtagtataatcatagctcaccgtagccttgaactcctgggctcaagcagtagtcccacctcagcctcccgagtagcgtacaccatcaccccagctaatttttaaattttttcctagagatgaagtcttgctatgttggttaaactggtctcaaactcctggcctcaagtgatcttcccaccacagcttcacaaagcactgggatggattacaggtgtgagcaaccatacctggccctctattatctcatatatagcttattgttacatatgctgtaattatatatagttataattatttaatacatatatgtttattacaaatatgcctgttaatggtatattctgtttttaaaaggaaggataattacttttttctatgagtaccttaagggcaggaactgagttttatttgtgtttgtaatttggctctgggcagaattcttggcatagtacgtgctttatgtgtgaactgaattatatttcttcatcttaattttacaggtgtgagccactgcaccaggccccttcatcttaattttaatatatctttgaataaacaccattgtatgaacctgctgtaagcttgggagtggtctgttagtctacagcttgtgtctgagatgtgctaattgaatatttgctcagtacctcatcttaactgcctttggctttatgttgcttatccttcatagtatcttgttcattggccttttacatccataggcatcacttctctgatattcgttgtgctcttttaatggattaatggtttgcttggttggttcctctagttagactgtaaactccttgagagcagagtctgtattttattaattacccacagtactaggtacatagttgccttcaataaatatatatttaatgaacgaatttagtgaataatggctacatttttgtacttcgttattatttatttattttttttttctgagacagagtcttgctctgtaacccacgctggagtgcagtggtgtgatcttggctcactgcaacctccaccgcccaggctgaagtgattctcctgcctcagcctcccgagtagctaggactacaggcgtgcaccaccatacctggccaatttttgtatttttagtagagatgggattttactatgttggccaggctggtcttgaactcttgacctcgtgatccgtctgcctcggcctcccagagtgctgggattacaggtgtgagccaccgcgcccagccttcattattcttttgacatttgatgataaatactgtttatgattttctttttctttttttttttttggtagagacagtctcgctttgttgcccaggctggtcttgaacttttggactcaagcgatccacccacctcagcctcccaaagtgttgggtttacatgcatgagccacctcgtgtgtccttgttcataattttgatgggctgggcgcagtggctcacgcctgtaatcccagcactttgggaggccgaggtgggtgaatcacttgaggtcaggagttcaagaccagcctggccaacatggcgaaaccccgtctctacgaaaaatacaaaaattagccgggcgtggttgtgcacgcctgtaatcctagctacccaagaggctgaggcaggagaatcactttaacccgggagatggaggttgcagtgagctgagatcgtgccattgcactccagcctgggcaacaagagtgaagctccatctcaagaacagttttgatgaaattgtccctagatttgcatctggcatctagctcatatctcagagccagaatgaaggatgcttacctctgggtttggtaaattttagcttggttaattatgtgtgtgccttaatttttttgacctattataaaagcaatatacgtaaatgcaacagagaaaatgaaagtaatccataaaattctacctgtggccagctgcgatggctcatgcctgtaatcccagcactttgggaggttgcggtgggtggatcacttgaggtcaggagttcgagaccagcctggccaacatggtgaaaccctgtctctactgaaaatgcgaaaattagctgggcatggtggtgggtgcctgtaattccaggtacttggggaggctgaggctggagaattgcttgaacccgggaggcagaggttgcagtgagccaagattgcaccactgcactctagcctgggtgacagagactcttaagaaaaagaaaaaaaaattcagatacctgtaacctaaataattaatatttgaaaaaatttcatcattgcttctttcattcagttaatatattttgaaccacttgccatggtgttatatatagacatgtcttatatatgtgtatatatatgtgtgtatttatttattttttgagacagagtctcgctttgtcatccaggctggagttcagtggtacaatcatggctcactgcacccttgacttcctgggctcaagcaatccttccagcttccctagtagctgggactacaggcgcatgccaccacacccagctaatttttgtagtttttgttgaggtgggaattcactagtcttgaattcctgagctcaagcaatcctcccatcttggcttcccaaagtgctgggattataggcatgagccactgtgctcacccagttttttatttttagatacagggtctcgctctgtggcttaaagctagatggtgcagtggcaccatcatagctcactacaaccttgagcgcctgggcttaaggaatcttcctgtcagcctccccagtagctgggaccacaggcatgcaccactgcacctaattaaaaaaaattttttttatagagatggagggggtctcattttgttcctcaggctagtctcaaactcctggcctcaagcaatcctctgcttcagcctcccgaagtgctgggattacaggtgtgagccacggtgcccggccccttattctttttgaaggctgttttaagaattccagatttccagccatgaatgtgtgattactaactagtctcttgttatatgcctcatcaacaccacacttctcccagaagttgtgcaggcatttagtttattgttactcaggaatggatgagaaaggatatcaaagatctgaaccagctgattaccatcttggtcacttgagaactcaggtctgtccaataaacaccctaatccaaggtggtgttaaatacatatatatatatttttactttacgtttatttattttgaaaaatttcaaacctatagaaaaattgaggcagtaccatagtcttagtccattttccattacttagaatatccaaaagtgagtaatttataaagaaaattaatttatttcttacagctatggaggccaaggtcgaggggacatatctggtcagcgttttgccatgttggtcaggctggtctcgaactcctgacctcaaggcctgccttggcctcccaaagtgctgggattacaggcataagccaccgtgcccagccacctctgagcactttagatagtatgtcttaagaacaaagacatagcccggcgcagtggctcacgcctgtaatcccagcagtttgggaggctgagtcgggcggatcaccttaggtcaggagttcgcggcaaaaccccgttactactaaaaatacaaaaaattagctgggtgtggtggcgtgcacctgtaatcccagctactcgggaggctgaggctggagaatcacttgaacccaggaggcagaggctgcagtgagccgagatggcaccactgcactgtagcctgggtgacagagcaagacttggtctcaaaaaaaaaaaagaaaaaaagaacaaagacattttcctatataaccacaatgccgttatcacactcaagaaacttaacattgatatgattgtgtataatacatagtccatattcaaatttttctctcatttcaaaattattctttgtggcctaaaatcctgtatccaatcatgatatttttgaacaatccagactagttgttgtatagaagtaagtagtttgttttttttgttttgttttgttttgttttttgagacagggcctggctccgttgcccaggctggagtgcagtggcatgatcacggcttactgcaacctctgcctcctgtgctcaagcaatcttgtgcctcagcttcctaaataactggaattagaggtgcgtgacaccacgcctagctaatttttgtatttttaatagagatggggttttgtcatgttgcccaggctggtcttgaactcctggactcgaagcaatcctcccacctcagcctcccaaagtgctgggatttataggtgtgagccaccaggcctggcagttaagtacattttttaaggactaataaaaatgtagaaatcttcgttatttccggaatcctttatgttttactgccagtactaatgtagtaatgccagacagtagtcatgtctactgttccaggttgtccaaaggattcagcagcaaaggatgaaagtggtgggaagttttgcatagtggtcgaccaagggctagtttggtgagtgtcttgcctatgataatggacaggaagacaccagttattttattttatttttattttattttattttattttaattttattttgtgttgtgttgtgttatctttgagatggagtctcactgtatcacccaggctggagtgcagtggcgcaatctcggctcactgtaacctctgcctcccgggttgaagtgatcctcctgcctcagcctcttgagtagctgggattatatgcgtgagccaccacacccgactaattttttgtatttttagtagagacagggttttaccatgttggccaggctggtcttgaactccttacctcaggtgatccacccaccgcagccccccaaagtgttgggattacaggcgtgagccaccacacccagctgacaccagttatttaactcacatttctctgttttccttttaaaggtagtagtgacacctttgaaatcttttggcattttctttgcctgttctcccttatgtgttaaggataagcttacgtgcagaatggcttgttaagttttttctcctccgtacttggaaatctcatctggtgggacattagggtcagaattttgtatattatatattatatatctatcatgatatatataatgtattttatatattatatattatataaatatcatgatagatatataatatataattatatgttatatattttatatttataaaataaaaataaaaaattgcaattattttcaaatagtaaaaccacgtagataaaatataattctggccaggcacgtggctcatgcctgtaatcccagcactttgggaggctaaggtgggtggatcacctgaggtcaggtgatataatatacaacagatataatatgtctattatatattatatataatgttatatagatatctatattatatataatatattaatagatatattaatataatatatattatatattatacagatatctataaatatattatatataatatattatagatatatctatgtatattatatcacctgacgtcaggtgatccacccaccttagcctcgcaaagtgctgggattacaggcgtgagccacgtgcctggccagaattatattttatctacgtggttttactatttgaaaataattgcaattttttattttattttttggttctggaaacatttgtgtggacttacctatgattgtcaggtttactgatttgctggagattttttgggcatgacaaagggtttcttctttgataaaagtttttctgggtaatgtgatggtacaggttgagtatcctttatctgaaatgcttgagacatgaagtgttttggatttgagaatttttagcatcttggaatatttaccacatacatagtaaggtaccttgaggatggggcccaagtctaaacacagaattcatttgtgtttcatatttatcttatatgcatgaaacaaagttgtttgttttcttttttttttgagacggagtctcactctgtcgcccaggctggagtgcaatggcatggtcttggctcactgcaacctccgcctcccaggtccaagcgattctcttacctcagcctcctgcgtagctgggattacagacatgaggcaccatgcccggcaaatttttgtatttttagtagagatggggtttcaccatgttggccaggctggtctcaaacccctgacctgaagtgatctgcctgcctcagcctcccaaagtcctgggattacaggtgtgaaccaccgcacctggccccaaaataaagttttgactctgtgtgtgtgtgtgtgtgtgtgtgtgtgtgttttggagacagagtctcactctgtcgcccaggctggagtgcagtggcatgatcacagctcactgcaaccttagcttcctgggctcaaatgatcctcccactttagcctcccaggtagttgggactacaggtgcgtgccactgtggctgagtaatttgtgtgtgtgtgtgtgtgtgtgtgtgtgtgtgtgtgttttaatagagatggggctttgccatgttgcccagcctggctcttgacctcttgggctcaagccatctgcccaccttgacctcccaaagtgctaggattataggcgtgaaccaccatgcccagcctttgactgtgttttgatggcaacccatcacgtgaggtcaggtgtagaaatttccatttttggcgtcaggttggcactccaaaggtttcagattgtggagctccttggagttttggattagggatgctcagcctgtacatttgacagtttatttacagatgcttgcatcagtgcttggagctagtcctcagtggtgggataggtgaaaattatctctactttgagttcttgatttgaattcttttttcttaagcttttcctaaagtcatgtgtcagtgttgattattgcccggttagaggtaggatttgtattgtttttttacacttggaatttcctgttgtcataagatcactgtgaactcccttgtagtgttgtacattcttaggactttcatgctgaaatactatgcttttataaaatggttgttgtgtctttgctgagtttaaatagggtccaggccaggcatggtggctcatgcttaccctgtctcaaaaaaaaaacaccctcaactaaatatagttaaataggccaggctcggtggctcatgcctgtaatcccagcactttgggaggcggaggcgggcggatcacctgaggtcaggagttcgagaccagcctggctaacacggcgaagccctgtctctattaaaaataaaaaaaaaacctagccaggtgtggtggtgaacgcctgtagtcccagctactcgggaggctgaggcaggagaatcacttgaacccagagacagagtgagactctgtctctcacacacacacacacacagataaataaattgggttcataaatggtgattccagcactcactgtcaggtataacttcagtctttttttttttttttttttttttttttttttttttttttttgagacagagtctccttctgtcacccaggctggagagcagtggtgtagtctcagctcattgcaacctccaccttctaggttcaagcagttcttctgccttagcctcccaggtagctgggattacaggtgcagaccaccacgcccaactaatttatttatttattattattattattattattttttagtagagatggagttttgccatgttggtcaggctgactttgaactcctgacttcatgtgatccacctgccttggcctcccaaagtgctgggattacaggtgtgagccactgtgtccagccaacattattcacttttgttgttacataagatgtcacggccaggcatggtgggtcacgcctgtaatcccagcactttgggaggctgaggtgtgtggatcacctgaggtcaggagtttgagaccagcctggccaacctggtgaaaccccgtctgtactaaaaatatgaaaattagctgggattacaggcgggcacctgtaatcccagctactagggaggctgaggcaggagaatcacttgaacccgggaggcagaggttgcagtgagctgagatcgtgccattgcactccagcctgggtgacagagcaagactgcgtctcaaaaaaagaaaaaaataaaaaagatgtcagttatggctatttgtactttattatacttcatgtgatgatatatggttcagcatcagggtcttttttcagttaagttcagttgaacccttttcctgtttagaatattttttaaaagtataaaaaaacttaataaaagaaatggatggctgggcatggtggctcacccctgtaatcccagcactttggtaggctgaggcgggcagatcacctgaggtcaggagttcaagaccagcctggccaacacagtgaaactccatctctactaaaaatacaaaaattagctgggcgtggtggcggccgcctgtaatcccagccacttgggaggctgaggcaggagaatcccttgaacccgggaggcggagattgcaatgagcctattgcgcaactgcactgtagcctgggcgacaagaaggagactccgtccctcccataaaaaaaagaaatagatgactacatttaaaatatcttccatttaaatccatagtttacaaataaaggaaaaagcagtggaggaagagagtgagataattacttcatttttacaatgttaaataattacaattaaaatttttgtgtacaattccacatttaatagcaatttgcaaatgccttattgccaggtgtggtgtcttacgcctgtaatcccagtactttgggagcccaaggtgggagaattgcttgagccctggaattcgagaccagcctgggcaacatagcgaaacttttgtctctacaagaaataaaattagccaggtgtggtagcctgcatctgtagttggaggtactgggggagctgacgtgagaggattgcttgagcccaggaggttgaggctgcagctgacatcaagccactagactctatcctgggcgactaattgagaccctgtctcaacgaacaaagaaacaaaaaaaacccaaatgatttacttaccattaaataccaagaaaagaatgactggagtaagcttagatttagatacaacaaaatcatggagcttatcatttgaaaggcatttttgacttttcataggtgtgtagcaagcaaagtggtccttttgaaaaatgcaagtcacatcatgtcacttttctgcttggcctttgcctacttctcgaatggttctcctagcacattctaactggtcattattcattgtgtttcagccattctggccttcaacctatttctcaaacaccccaatcttgggtatactttagggcccttgcatttgctagctcatttacctggaaaattctgcggtaacttcttgcacataataggtgctaataaatattggctgcatgcaaccaaaaaagctggcttcggtagtagaagtagtagaagagaacattaccaaagtccttttttttgagacaagatttccctctgttatttaggctgagtgcagtggtgccatcacggctcactgcagccttgacttcttggactcaaatgatcctcccacctcagcctcctgagaagctgggaccacaggtgtgtgtcaccatgcctggccaatttttttatttgtagagaagaggtcttactgtgttgcccagaatggtgtcaaactcctgggctcaagtgatccacctgcctcagcctcccaaaatgctgagattataggtgtgagccactggtccagcctcttaccaaaatctttttttttttttttttttgagacggagtctcgctgtgtcgcccaggctggagtgcagtggcgggatctcggctcactgcaagctccgcctcccgggttcacaccattctcctgcctcagcctcctgagtagctgggactacaggcgcctgctaccacgcccggctaattttttatatttttagtagagacggggtttcaccatgttggccaggatggtctcgatcttttgacctcgtgatccacccacctcggcctcccaaagtgctgggattacaggcatgagctaccgcgcctggccgcctcttatcaaaatcttaaacttgttaattcttgtatctcaccttgcttttatatcagtttttgttatagaatgtgctttctttttaggttatttttcttctgctcagcaagttaactttgaagaatcttttttttcttgccttttttttttttttttgagacagaattttggttttgttgcccaggctggagtgcaatggcgcgatctcagctaacggcaacctccacttcccgggttcaagtgattctcctgccccagcctcccgagtagctgggattacaggcatgaggccaccaggcctggctatttttattttttattttttatttttattttttactagagacagtgtttttccatgttggtcaggctggtcttgaattctcgtcctcaggtgatccacccgccttgacctcccgaagtcctgggattacaggtgtgagccagcgcacccggcctcttgccctctttagagcagttctacataaatctgtggaataatctgctgacgttagaaataaacctttctcagggtgtggccataatctggttcgttgtaattgctgaaggaaaaaactgaacccttaggaacagtttaaacattctaggtggtttttagatatattattcctgttagagttttgaactttgatcatcttgttagaaaaaagaggccaggcactgtgcctcatacctgtaatcccagcattttggaggctgagtcgtcaggaagtcacttgagcccaggagtttggtaccagcctgggcaacatatggagacctcgtctctacaaaaaaaaaaaaaaaaaaacaaaacctgttagccaggcgtggtggctcatgcctgtgttcccagctactcaggaggctgagatggaaggattgcttgagcctgggaagttgcgactgcagtgagctgtgattgtgccactgcactccagcctgggcaacagagtaagaccctgtctcaaaaaaaatgaatttttgcattcatttaaaaatgaatggtagcaacattttagataataacttatgtagtctgtgtgaatctcatcaaatatatgtgatttttaatgcttggaatcctagaattttagtgagggaggagctctcaagttatctaatttactgtcattttacagataaaaactaaacttctgatagatgaaaggatttataccatacatctatatgataattgcatatggctgagttgaggttagaacccaggacccaatctttgatttgatttcttttcattatgccaatttagaataaaattttttctcttggttttctgacctttggccacttcatcttttgaaggatcttgtaagttccttgatatttctcaaaatgtcagttttattctcttcaatgaaagttcttgttaggtgaaagtagcctttcatatccttaaatgtgaacctgttccttaggctggagtttttgtactctccattatattggcatttatagttgttggttttcttttttttttttcttttgagacggagtctgtctctgtcgcccaggctagagtgcagtggcgcgatctcggctcactgcaagctctgcctcccgggttcacgccattctcctgcctcagcctcctgagtagctgggactataggcgcccgccaccatgcccggctaatttttttgtatttttaatagagacggggtttcactgtgtttgccaggatggtctccatctcctgaccttgtgatcctcccgcctcggcctcccacaagtgctgggattacaggcgtgagccactgcacccggccagttgttggttttcttttctgtgccagattgagtgatatatggtggtactaagagcactggaataggagtcaaggactgaggtacagcatagcacctcatgtattacttggtatctccacttgtctctaacactgaagaggtataattggttgtttaaaaactgtgggtgtaaaacgctagtatcccatgaaatggaactgcagaatataagtggatgactctgaaactgcttttgaaacaagcatgagggctcagtttctccatctctcgtgggctccaccatcactgtctgacatcctgatctcctaaggcatctttcctccatcccagagctctgttgaaaatctcttgggctaaaaaccagtacagaccttctgatactcacgttctagcctttttagattgttgtgtgtgaagagtaagtcagctgtggatatttttctttcctattttgtgaggcatttggtatttactggaggctgcctgttttgttgcaaaccttgttccactgtgtactataggaggtctgctcagaaagcctagttagtagagcagtaccatttgctttggtcaattgtagcagcagtctgttttttttttttttttttttttttttgagacagagtcttgctgtgttgctcaggctggagtgcagtggtgtgatctttgctcactgcaacctccgcctcctgggttcaagcgattctcctgccttggcctcccaagtgggattgcaggtgtgtgccaccatgcccggctaattctgtatttttaatagagatggggtttcacccccaggctggtcttaaactcctaacctcaagtgatccacttgcttcggcctcccaaagtgctaggattacaggtgtgagccaccgtgcccggccagcaatctggttttaaaggaggctttgacccctcatttggtaaaaggttccttctttgactaattcccaaaggacatctcgatctgtcttcaaaagaaggggtattccgttgtcagtattcctagctagctctgggagaagagagaacttgattgttttcagggccctggtcagagtctggttttatgcccaagggatgaactgccttttgtccttctacctcctgaagttcagagccctgtgaaaatgaagtggtaaaggtcaagtagcagtagtttgaaaagcaccagcactgggacgattttgtggtagcctagcagagtgggcaggatgcttacttcagaggtgctaaagatgtatttttttttggaacaagaaggttataaatgtttctattaagtttgattttaaagcatgagggttctgggaaaatgagaagattgtacatactctaggcttccttgggactgcatttgaaaaaaaaaaaacttttctttgggtcagccagtatgaaaactgcatttgatctcaatccttcagatcaggcttttggggagagttagttgaagaatttgtttagtctaagaaagttgcttctagcttagctaaatctggaagaaaaaatggaacctgatgtttgagtgctaactgtattccagacagacgttatactccttatattggtccttttgaagcataaatatgtttccaagtcctataataatgaggaaattgcctgtgagagtttaagtaaattgtacaaagtaacacttttggttcagagatttgtttttaatacagagaggacaggttaggaactttgaagcttcagctaaatgagtcagttctcaaatgtttgagaagcatgctcaaggatctggaggaatagcttaattaatttgtacatcatgagaggagtagggagcaaaaatatgtataatttcttctgaatgatgctgtcttttttgatgcagtggtgctaaatgggctttgcttgtatgatacttcttaaatgcattagctaattattttgttttagtcttcttttttgaaatacagatgactctttgagctggttttctttggaaagttatctgtcctgacaattctacttctaaaacttcaactttttttttttagtcttcatttccattgccaccaccctatcctaaattatcccgttatgctggatactgaaatagcttctgcctggtctacttactttcacttcgcccccttacgatccagtgctttgctccttttaatccagtggccagagggatttttttcaaaacataatttgattataacagatctatgtttgatacttatccatggtttcccatggcagttgcagaaaaatccaaaccttgtttggccacactgcccttctttcagtaatttgaattccttaagctttttccagtttcaggatttcccattttgaactctttctcctccactttttcctggccacaggtttcaagagctgacaacttacctcagttcttaagaggatccttcatttatctctaaattggattttcctgtgtccaaaccttacttgttatcacaagtttataattatttgggtgattgtttgatgtgtctctcctgactgggccttaggggcagagaatctgtattattttgttcttgtttatatccctaggagctcttatggtaggtattcaataaatgtttgttgaataaaggcagtggaaaaacacatagaagaggtgctgtggcagcagttctcagtgccaacagttgaattggtttacaatagtaatttttagtgtcaacaaggagatgtgtagtatggaatgtttattagcaagatctttttctcatggaatggaatgtttgaaaacctccaaagtgtaagtttttttttttctcaaaagaaaacagtcatatttggggttacttttttttttgagacagagtcttgttctgttgcccaggctggagtgcagtggcatgatcccggctcactgcaacctccacctcccgagtttaagtaattctcatgccttagccactggaatagctgggattacaggtgtgtgccaccatgcccagctgttttttgtataattattagagatggggtttcgccatgttgaccaggctggtctcaagctcctggcctcaagtgatctgccagccttggcctcccaaagtgctgggattacagatgagagccaccgtgcccggcccattgttacttctgaaatcgaaatcaaatgccatattgattcctgatcttttgtgtatgacctctctctctcttttttttttttttttgctacctctccccgaaagtttttaggatctttttttccacgattatgtaccttgatgtgggtcttttctcgccctttgtgctattaggtctagcaactcatgtgtctcaggtctgggaaatgattttacagtttttccagctggtgcgtaactagctgtttgttctgatactgtcagtatttaactttatgttagaaacatttcataatgcaaatgaaaaatttaagccaggtgcagtggctcatgcactttgtgaggctgaggtgggaggaacacttgaggtcaggagttcaaaaccagcctggccaacatggtgaaaccccttctactaaaaaaacaaaaaaacaaaaattaactgggcatggttgtgcacccccataatcccagttactcaggaggctgaggtacgagaatcactggaactgggagacaggtttcagtgagccgagactgagacagagcaagaccctgtctcaaaatataaataaaatacataaaaaatttaatattatgtatataatatccagcccaggcttttctatttgtcatgccctgtttctttcacatcatagccttactcagatgcctgctgatccttgattgaggtgtgatgcttgagagtcaggctcttaaatactactggcgaattcagtgcattgtggttttgtgacttcacttgtctcttacagaagtggggaagtggtttttacttcgatgcatggttctgaagtagagatgtctatctcctgatgcacactttaaataatccccatgttttccactccatactccatccttgtctggtttaagctcttagcctgtctgcagttcaggggatgaatctgcttggttcttttcagcatctccctctgcaacgcttagatctcatcttcctttgctttgctaactattctctttgcttctccatttgtttctctgtcattgatgacttccaagttatatccccatcctggatattccttttgaacgtcagactcatatattcagttacctcctcagtatctctattcggatatctaatggcatctcaaaattaacatgtgcaaaaccaacctgttccacccttgtgttggctgtataaattagtgggaactccattttttaagttgcttaaatcagaaacctgggagtcatcaattttggagtctgtccctttgctcttgtggcacatgtaatcacgaataaatcctgttggctctatcttcagaatatatctcattttatcactctctctgccaccagagtggtttaagccaccttcatctcttaggttattttagtagcttcctaactggtctccttccttccattcttgccaccttcctcaacaccgaagatatattttatttatttatttatttattttgagacagagtcttgctctgtcaccccagctggaatgcggtggcttgatcttggctcactgcaacctctgtcttccaggcttaagcaattctcctgcctcagcctcccgagtagctggggctgcaggcatgcaccaccatgcctggctaatttttgtattttttagtagagatggagtttcaccatgttggccaggctggtctcgaactcctgacctcaggtcatctgcccgcctcagcctcccaaagtgttgggattacaggcgtcagccaccatgcttggccaaattttttattcctttgggtaaagagatgtattttatttcctttaatttggtaatatagtgagttatgttaatagattctttttttccagtgctcaagcatccttgcattcctaggataaatggtacttcaacatgattttccttttaaatatacattgctggatttaggttataaattattgagaatttttgcatctgtatttacaagtgttacattctaatttcaggccttttccagttttattatcaaaacaagctgtgctgtttttcctctcttgattctcagtagtagtctctttaagatagtgattattcattctttttggtagatcatgcctataagacctgggcatgtatgtttttattgtgggaatatattgtttttactttccttgttatgaaatatataatttattttaaaatatgtatatacagagtttgtgttcctttaaagaataatagctaaaaataaattgcacgcccccacatccaaccatcgtcaaacaaaacatcttttacactctattttatttatttattttcccccacccctctagagtatacacccttttggagcctctgagcattttcctttccctcactgctttagaaacctctattcagatttttcatattaatgattcttttgcttttaacccacatgtgttactgaagtttaagtcctaaataataatttttcctgctttaaaatatatttttatgtcaatgaaatcatactggtggatgtattttctctgatttgctgcttttgcctctcatttctgagcttgatctatgttggtgcatttatagaaatacttcatattttctgctgtgtatcatcttatgctatcaacgtgccatatgatttacctgttcttagcatgtatttgggttgtttctggttcttgctgtaacaaacagctgcatttagtgcttgtgtatgtgtctcctgatgcatgtgtgcattcacttctctggggtgtatatgttgctgggaatgggactgagtattgtgagtgtataatagtatcttgtttttgttttttttccctctcagtttatggcataatttacgtacagtaaaatttaccaccgcccccctgccccgcctttttctttttctgaaacagagtctcgctctgttgtccaggctggagtgcagtggcacaatctcggctcactgcaacctttacttactgggttcaagcgattctcctgcctcagcctcacaagtagttgggattacaggtgcctgccaggctaattttgtagagacagggtttcaccatgttggccaggctggtctcaaactccttacctcaggtgatccacccaccttggcctccccaagtgttgggattagaggtatgagccaccacgcccagccaaattcaccctttttagtgtacaagtatacagctttgtgagagtttgatttctttagttcaaatttgttttaaagttatatttggtattttaacagtgctatttaaatatatgccatatatggcttgaatgcaattatcctagtacagatggtccttgacttatgatgtttttgtgtccctgtaagcccaccttaagttgaaaatattaagttgaaaatgcatttaatatgactaacctgctgtaaatcctaacttagcctaccttaaatgcacttagaacacttaaattagtttacagtgggcaaatcatcaaacacaaagcctatttgtaataaagtgttgaatatcttatataatttattgaatactaaagtgaaaaacaatagttgtatgagtagtcagagtatggcttctcttcaatgcatatcactttcatataccatcgtaaactcaaaaaatcataagtcagggtatgctgtatattttattggtgctaacatacttccattttaaaatacaggaaattcacctttttttgtgtatagtcttgtaagttttgacagatctatatacagatatgtaatccccacccatgattcttttttaatttaaaaaaatatatttttgagacagtctcactgtcacccaggctggaatgcagtggcatgatcgtagctcactgcagcctcaaactcctggccttaagtgctcctcctgcctcagcctcccaaagtgctaggattacaggaatgagccagtgtgccctgccaccaccataattaagatccagaacagtttcatcatcccagaaactttccctgtatccttttgtagtcagcttcttttttcacccccagctcctggtgaccactgatttgttctctgtccttgcagtactgtcttttccagaatgccatataaattgaatcctacattatataacactttgagtctggcgtcttttacttagtgtgaagtatttgagatttatttgtcttgtgatgtgtattagcaatttatttcttttttcttttttttctttttggagacaaggtcttgctctgtcacccaggctgaagtgcagcggcacgatcatagttcactgcaggcttaaactcaagcaatcctcccacttcagcctcctgagtagctgggactgcaggcgcacaccaccatgttcagctaattttttttttttcttggtagagacaggatctcactctgttgcccaggctgatttcaaactcctggcctcaaggaatcctcccacctatgcctctcaaagtgctgggattacaggtgtgaaccattacagttggctagcagtttatttctgtatattgcaggatagtatctcattgtatagatgtaccacattttgcgtattcattcttcagttgatagacatttggactacttcctccttttggctgctgtgagtaatgctgctgtgaataaatatttgtgtgtaagtttctatgtggacatacagtttcattattcttgggtagataaaccttggagtaattgctgggttgggtggtaatttctgtttaacttttaaagaaatagctaagctgttttccaaagtggcttcaccatcttaaattcaagtttttgttttcgttgtatttgaggcagagctttactctcttgcccaggctggagtgcaatggcaagattatattatagctcactgcagcctcgatcttccaggctcaagcgatcctctcacctcagcctcccaggtagctgagagtacaggtgtgtgtcaccatgcctggctaagttttgatttttagtagagataaggtctggctatgtttcccaggctggtcttgaacttctgagctcaagcagtcctcctgccttggcctcccaaagtgctgggattgcaggtgtgagccaccaagcccagccaaggtttttgtgttttagtaaaatttctgctcttcctggcaatacttttttctgaaatttactttgatattgatatagtcactccagttcttttcagtgcttagtttttgcatggtatattttttccatccttttgtgttatactgtctgtttcattatatttgtagttggaccttgattttttttttttccctaattcttttacttgggatgcttggacctaatgtgtgtctagtttttcctaatgtgtcttttacttgggatgcttggaccatgtgtatttaattattgatatggttgggttaagtccaccatctagctatttgttatttatttatttttagagccaaggtcttgctgtgccacccaggctggggtgctagtggcatgatcatggctcactgaagcctctaacttctgggctcaagcagtcatctcactgcagtctcccgagaagctgggattacaggcgtgtgccaccaagcctagctgattttttttttttttttccgagatggagatttgctcttgttgcccactctggagtgcagtggcgcaatcttggctcaccgcagcctctgcctcctgggttcaagcaattctcctgcctcagtctcccgagtagctgagattacaggtgcctgccaccatgcccagctaattctgtatttttagtagagatggggtttcactatgttggccaggctgatcttgaactcttgacctcgtgatctgcccgcctcggcctcccaaagtgttggaaggccgaggcgggcagatcacgcctgtgctgggaggctgaggcgggtagccactgctcccagccctgaaatctttcctcttttctaagatagacatttaatgcacacacacattttgatatgctatgttttaattttcattcagcttaaaatatctaatttttcttttgatttattctttaacttactgattatttagaagtgtgatattatattttgggaataaaaatccagagatcttactgttactgatttttgatttaattccattgtggtaagagaacataccttatatgatttgaatccttttaaattgattgagacttgttttatggatcagaataaggtgtcttgataaaggttccacgggcacttgaaaagaatgtgtattctactgttgttaggtagagcattctattaatgttgattggcttgttgattgataatgttcaagtcttctgtattcttaccgatttcctattctatcagttatagacagagggtgttaaaatctcaattctgttttgcttgttgattgataatgtttaagtcttctgtattcttaccgattttctgttctatcaattatagagagaggtgttaaaatctcaattctgtttgtttttggtttgaaatttacttataaggtacataagcatttatgattgtcgtcatcttgatgaattggtctctgtcattttgaaatgacagaacttactttgtctcataatatagctactcgtgcttttcatttgattagtgtttggcatatatttttatattttaaaaaaacttatcacctgtgtctttatgtttaaagtggttttttttcccaggcatcatgtgtttgggatcttgcttttatccagtttgatagtgtttgccttttaattggagtttgtagactatttatgtttaatgtgattactgatttttttttgagatggagtctcgccctgtcgcccaggctgcagtgcagtggcacgatctcagctcactccaacctccgtttcccgggttcaagtcattctcttgcctcagcctcccaagtagctgggactgcaggcacctgacaccacacccggctaatttttgtatttttagtagagatggggtttcaccatattggccaggctggtctcaaactgctgactttgtgatccgcccacctcagcctcccaaagtgctgggattacaggtgtgagccactgcggctggatgtttactgatttttatagagtttaaacaaccatcttttctatttgtctgatgtggtgtttttttcgtttttgtttttttaagatggactgtcactctgtcgtctaggctggagtgcagtggtgcaatctcaactcactgtaacctttgcctcctgggttcaagcagttttcctgtctcagcctcccaagtagctgggattacaggtgcgtgccaccacacctggctatttttttttttttttttttggtttgagacagagtctcgcactgtcacctgggctggagtgcagtggcgcgatctcggctcactgcaatctccgcctcctgggttcaagcgatactcctgcctcagcctcccaagtagctggtattacaggcgcccatcaccacacccagctaatgttttgtatttttagtagagacagggtttcactatattggccaggctggtctcaaattcctgaccttgtgatctgcccgccttggcctcccaaagtgctgggattacaggcgtgagccaccgtgcctggcctaatttttgtatttttagtagagatgggcttttgccatgttggccaagcaggtctcgaattcctgacctcaagtgatccctgcccttggcctcccaaagtgctggcattacaggagtgagctacgccatgcctggcctggattgaggattttttagtggttcatttttaagtcctttcttggctcattagtactttgttattttagtggctgctttagtgtttattgagttcagcttgtgaaactcagtctttaatattataccacttcacctatattacaagaaccttatagcagtacacttcattttcccttcagactttgtgctatgtttgtcatacacttcacttttacatatgttgtaaactccacaatacattattatttttcatttaagtagtcaatttttttaaaacaatttttttttttttgagacggagtctcactcggttgcccaggctacagtgcagtggtgcaatcttggcttactgcagcctctgcctcctgggttcaagtgattcttatgcctcagcctcctgagtagctgggattacaggtgcgccccatcatgcctggctgatttttgtattattagtagagatggggttttgccacgttggcccagcaggtctcgaactcctgagctccagtgatccgcctgtcttggcctcccaaagtgctgggattacaggcatgagccaccatgcctggcctaaaaagattttaaaagtaagaaaaaatgtcttttactttatatgttttccatttctggtgttctttattgcctatgagcggttccagattctcatctgccagattttcttcagttttttctcgtcagttcagattttctttctgccagaggacttcctttaatgtgtttcttgtagtgtaggtctgctggtgatatctttaagctttggtgtgtttgaaaaagtacttcctcttgtttttagaaagatatattcctttgatacagaattctaggttgacaggtttttttcattgtgtactttaaagatgttgctccactgtctctagcttgcattatttctcctgtaaaatctgttgttattttaatctttgttcttcagcgagcaaatttatcttttttgccttttttggttaatttttaagattttctttttatcactgctttttcagcaatgtgtttattatatactttgttgtagtttgcgtcatatttcttgtgtttgaattctcttgagcttcatggatgaaatttacgtattcagctactatttcttgaaatatttttcttttgtacttccccaccgcatctggagtctgtaattacatgtatatttggctgcttgaagtagtcatacaactcactgatgcactgttatgttttttagtcatttgattctctttgttttgggtagtttctactgatcttcatttcactagtctttatctttcccatgttaaatctgttactcttatccagaattataccttttatccttaaaaattcagtttttttcttttttttgagacggagtctcattctgtcacccaggctgaagtgcagtggcatgatcttggctcgctgcaacctccgcttcccaggttcaagcgattatcctgcctcagcctcccaagtagctgtgattacaggcatgcatcaccatgtctggctagtttttgtatttttagtagagacagagcttcaccatgtttgccaggctggtctcaaacccctgacttcaggtgatccacctgcctcagcctcctagagtgctgggattacaggaatgagcgaccacacctggtttgtttcttaaaaaacatcttatatttctctgcttaacgtgctcaatgttgaacatgtgaaatataataacttttcataatctcttctgattctgtctctatatcatctttgggtctgtttttatgtattctatttttcttgtcattatgggtcagatttcccagttctttgcaatcctaataaattctgtttagatgaaagacattgtgaatttcactttgggttgtggatatttttgtattcatgttggtatttttgagatttgttctggagtgcagatgagttagctggaaactaattcttttgaggcttgctttttagctttgttaggtggggcgtatcctttctgtgtttatttttgtaagggtatacagatatacatgttttcttatttccctctttatttattcttatttaatctgtaacaatctttttccttatattcgtaacattcttagttagggagtaattacctcttccctaactgttgcattagcttcttaattggcactctggttcccagactttttctgtctctaatgcagtgtacagactactgtcagaataattctaaagcacaattctgatattgtcactgtattgcttttcagtgcctacagcagtaggattcacttaaaaaattattcccctaatagtaatataaaaaatttttttgagctcaaactctcattctctctcagtatacatatttgtttatacatgaggtcacatactgcttactaagttagcattttttatgtgttcattttgaaataactctgaacagtggtatccaatcttttggcttccctgggccacattagaagaagaattgtcttgggccacacataaaatacactaatgatagctgaatagctaaaaattaaacattgcaaaaaaaaaaaacaaacaaaaaaaactcactgtggctttttttttttttccctgtatttttctttttcccgagatgaagtcatgctctgttgctcaggctggagtgcagtagtgtgatctcggctcactgcaacctccgcctaccaggttcaagcagttctcctgcctcagcctcccgagtagctgggattaaaggggtaagcccccatgcctggcctttttttttttttttttgtggggtgagcgaccatgcctggctttttttttttttttttttttttttttttttggtagagatggggtttcatcatgttggccagtctggtctcgaactcttgaccttgtaattcacctgccttggcctcctaaggtgttgggattacaggtgtgagccaccactcctggccttttttttttttttttttgagatggagtctcactttgtcacccaggctggagtgcagtggcatgatctcggctcgctgcagccactgcctcccgggttcaagcagttctcctgcctcaccctcccgagtagctgggattacaggtgtgtgccaccactcccagctagttttttgtatttttagtagaggcaggtttcaccatattggccaggctggtgaactcctgacctcaagtgatgcacccacctgggcctcccaaatgctgctggaattgcaggcgtgagccaccatgccaagccaattgtgttttaagaaaagtttatgaattagtattgggcagcatcgaaagtttacaaagtttgcgttgggccgcattgaaagctgtcctgggctgtgtgcgacctgctggccgcaggttggacaaacttgctctgggacataagtttaaaagttataggccaagtgccttggctcacgcctgtaatgtctttgcatttcatcagtttttccactgttatctttctgttacagtacacggtcagaataccacattgcatttagttgtcatgtttccttagatgtgaaacaaatttaccaattgtagtagggtatttacgtatagatcggttcgtttgtagccttatagtatctagttaaaacactgttataaagttattgttatgtatccttttgggtgtggtttctttccttagaaaaatacatttaaaattcattcatgttgttaaatgaatcaataggttgtttctcttttaaaaattgagatgaaattcacataacaaaattaaccattttatttttttaattttatatatattttctgagatggagtctcactctgtagcccaggctggagtgcagtgacgcgatctcggctcactgcaacatctgcctcctgggttcaagcgattctcagccttaaccattttaaagtgcacagttaagtggcatttagtacattcacagtgttgggtaaccatctttatctggttccaaaacatttttttttctttgaaagaaaaccccgtatttattaagcatttttctctctgtccttttcccttcactcgcttctggcaaacaccagtctgctctgtttctgtggatttacctgttctggatattttatacaaatggaatcatagcgtatgtagccttttgtggctggttttgttttttttgtttgtttttttttttgttttttttttttgagacggagtcttgctctgtcgcccaggctggagtgcagtggcgggatctcggctcactgcaagctccacctgccgggttcacgccattctcctgcctcagcctcccaagtagctgggactacaggcgcccgccactacgcccggctcattttttgtatttttagtagagacggggtttcaccgttttagctgggatggtctcgatctcctgacctcgtgatccgcccgcctcggcctcccaaagtgctgggattacaggcgtgagccaccgcgcccggcctggttttgttttagtaagatacttttgctggattaagaataaggttgggccaggggtggtggctcatgcctataatcccagcactttgggaggccgagttggcccagcctggccaacacagtgaaaccccatctttactaaaaatacaaaaattagtcaggcatggtggcgggtaccttgtaatcccagctacttggaaggctgaggcaggaggattgcttgaacccgggaagcggagtttgcagttagccaagatcatgccactgcactctagcctgggcgacaagagcaagactcgtttcaaagaaaaaaaaaagaatacgtagttgacaggtttttttttctttcaatactttaaagatgatataccactgtcttgtcactcaccttgtttctaacaagaaatctgctatctttgttctgtattttgcatgctttttttttccttgctgcttttattattttctctttattactggttttgagcattagattatgatatgccagtatggtttttcttgtgtttattgtgcttgtggttcattgatgttcttggatatgtcggtttatagctttcttcaggtttcggattttttttttttttggccatttttttcaaattattttccatttaatttctcttttctttttagtcaggtactctttatccaaacattaggctgcttgaagttgccccatagcttactgatagtttttgttttaacctgtgttttatttgaaagattctatttctgtgcctttgaatttactttcttttgcagtgtgtaatctgaagttagtcacatttagcatttttaaaaatctcacatattttactctttttttttttttttttttttttttttttttaaagactgagtctcactctgttgcctgggctggggtgcagtggcacaatttcagctcactacaacctccgcctcctggtttcaagcgatcctcccacctcagcctcccgagtagctgggactacaggcacccgccactacgcctggctcatttttgtatttgtagtagagacggggtttcgccatgttggccaggctggtcttgaactcctgacctcaagtgatctgcttgcctcagcctcccaaattgatgggattacaggtgtgagccaccatgcccggccatattttacctttttttatcgttagaattttgatttggtttaaaaaaaaatctttcctgtctctacttaagtttttgaatatatgaaatgcagatataactcttataatgtccttgtgtactcattctaacatctgtgtcatttcagggttggttgcaattgactattctctttagtatacttttttttccttgcctcttgcctggtgatctttgattggatttcagacatagagagttttacctcgttgggtgctggggatatttgtatttctacatatgttctttaactttgttattggatgcagttaagtcacttgaaaaccgttgattcctttaggtcttgtttgtaagatttgttagctgggaacggtgtagtagttggtttatggctaattcttcctcacttctgagacaagattcttcccaaatcttgagggttgtcttgagatttccagtctaatagctacttgagtgggtaagggcagagtactgttcataattcttttgggtgttgttttcctctccttgctgcccatggtcttgggtagtttcatcacactatgggccactcagtactcagtgagtacctctctagatctcctcagccctctactgtgtggtattgtcccatgaattctagctgcctaagtcctgcactctgagctgagctcgctgtcttcaattttgagttctgcagcctccatagggcttctgcttctgcatggtgcccaggaaactctcttcaggcactaagtttggccatcataggactcatatctttgtttctcttcctttggggattactgtctctcattgcctgctgcgcagcatcttgaaagctatagtttcatgttttgttagttattttagtggtttaaggtgagagggtaaatctggtccctgttgtgcttgaaaagtttttttcaacttttcttttagaaataattttaggtttactaaaagctgcagaaataatacagagttctgaggtgtctttcacccacttcccccatgataacattttacataaccatagtatagttattaaaactaggaaattgacattggtataatagtgttaactaaacctcatgccttatttcatattttcacatgaactattttgagctggcatggttaatatacagttctatgaagttttatcacgtgtatagatttcgtgtaattatcactagtcaggttatagaagtattccatcattttaatgaaatgcctttgtactactcctgtgtagtcatcatgtcctccttccagcctccaccaccaatcaaccaaccaactttaatcctggcagctaccagttgtctgaattttatcactttcgctataattttatctttctcagaatgtcataatacagtagtccccgttatctgtggtgatatgttctaagacccccagtggatgcctgaaaccagctatagtactgaaccatatatgtgtgtgtgtgtgtgtgtgtgttttttccccccccccctttttttttttgagacggagtctcactctcgtcacccagactagggtgcagtggtgtgatcttggctcactgcaatctgcacctcacaggttcaagcgattcttctgcctcagcctcctgggtaactgggattacaggttcgtgttaccacgcaggctaattttttgtatttttagttgaggtggggtttcaccatgttggccaggctggtctcgaactcctgacctcaagtgatccacccaccttagcctcccaaagtgctgggattacaggcgtgagccactgcacctggcctactatacatatacgcctatgatgaaatttaatttataaattaggcacagtaagagattaacaataataaaaaattataacaatatactataataaaagttatgtgaatgttgtttctctctcaaaatactgtaatattttcaggctgcagttgaccatgcgtagctgaaagcaagggtaagtggtgactactgtaaatagaatcatataatatgttacctttgagattggcttttcttttttaactcggcaaaatgcctttgaaatcgatccaagtcctatgtattaacactgtttctttttattggtgagtagtattctattcttgggcacactacagtttacccattcttccattgagagcatttctcccccctaaattacaaacagagatggagtcttgctttgtggttggtctccaactcctaggctcaatctatcctcctgcctcagcctcccaaagtgctgggattagaggcatgtgccactgtgcctggccccactgaagaacatttgagttgttcccagtttggggctattacaaataaagctgctgtgaacatttgcatgcagattttagtgtgaaaatatttttatttctctaggatgaatacctacctatgagtagaattgctgggtcgtatgatacatgtatatttaactctgtaagaaacgactacactgttttccagagtggtggtagtttttcattcccactaacaatgtataagaaatccaattgttctgcttttttattagcactttagtattgtcagtatttttccttctgttcattgtaatagatgcatagtagtatctcatgccttaaatttgcatttccgtagtggctaatgatgttgaatatcttttcatctccttaggttccatttgaatattctctttggtgtagtttatgttcaagacttttctattttcaaattggattttttcttactgttgagtgttctagacacaagttatttgttggatacgtgatttgcaaatattttctcctaatcttttaatttgcctaagagtgtgcttcatagagcaaaaggtttgaattcttatgaagtctaatttatctatttttaaattttatagttttttttggtgtcatatctaagcactctttggctaacaaaattttctctttttttctaaaagttgtatgctttacatttaagtctgtgattgcatttgaattaatttttaaattaagcataaggttacatcaatgttcattttcctttttgggggaccaattattccaaaaccatttgcttaaaatgattattctttatttattgaattgctttctcacctctagcaaaagtcagttggccatattttgtgaaaagttctattcctacgcacactgttctggctcattgacctacgcatctgttcctctgtcaataccgtactgtcttgattactgtaatgtagctgtatagtaagtcctaaaatcaggtagtgttattcctccaccttcgtttttctttttcagagttgtttcagctattctggtttctttgtctttacatggaaattttatttatttattatttattttcttgagacagcctcagtgttttgcccgggctggggtgtggtggtatgatcatagttcactgctgcctggaattcctgggctcaagggatcctcctgcctcagccttccgggtagctgggactacagatgtacaccaccactcccagctaatttttactttttttgtagtgctggggtgtcaccctgttgcccatgttggtctcaaacacctgggctcaagtgatcttcctgcctcagcctcccaaagtgctgggattaaaggcatgagccaccacagtcagctgtgtatgaattttagaatcatattatctacaataaagttttggtggagtttcaatagtaaatgcattaaacccattggttagtttggggagaagtgacatcttggccatgttgtcttccagttcatggatatagtatgtttttttacatttatttaggttgtcttttttttttaaaggtacataataattgtacatatttatggggtgcatgtgatattttgatacaggtatgcagtgtataatgatcaaagcaaggtatttaggctatccatcacctcaaatatttatcatttctttatgttgggaatttttcaactcttcccttctaggtattttgaaatataaacaaattatagttaactacagtcaccatactgtgctgttgaaccctggaactaatttcttctatctaactgtatgtttgtacccattgaccaacccctctttatctccctgcccccttcccagctctggtaacgatcattttactctctgcctccacgagatcaactcttttagctcccacataggagtgacaacatgtgatatttatctttctgtgcctcttcctttacttaacaatgtatttaggtcatctttggtatttttatcagcatttcataggttttagcatgcatattttgtacagttttgttagatatatacctagttttttttggtaaactattataaatgaaattgtgcttttaatttttgtttctaattgctgattgctaatatatagacattattgatgttagtgtgttgatctgtattctgtgacctttataaactcacttattagttctacaagtttttctcctagaatctttgggattttctctgttagaccatcatatcatctgttaatagggccggtttaatatattcctttctgatatgtgtaccttttatttccttttcttgcttgattgactagctgggacttgtttgctgtgtcgaatatgagtgcttagtgtggacattcttgccttattcccgctttcagatagaaagcctttagtctttcaccactagtatgatgttagttatagacattttatagatggtctttatcaagttgaggacgttttcttctattcctagtttactgagagtttttatcatgaatgtatttgttgaattttgtcaaagtttctgcattgatataaataattatgctttcttttcacatcttattgttttagtttgcatttccttgatgatgaattccttttcatatgcttactgaccattttatcatcttcctgcgtgaattttctattagttttgcccatttaaaaaaagtttttattttgtagcaattttaagctttcataaagttctaagtacagtacaaagaactttattttttcctgacccattttagagccctgttgttccttactatttttatttattttttgagacagggtctgtcactctgttgtacagactggagtgcagtggtactatcatagctcactccagggcataaacgatcctcctgcctcagcctcctgagtagctgggacaagtgtgtgccaccatgcctggctaatttttttaattttaattttttgaacttctgggctcaagcgatcctcttgcctcagcctcccaaagtgttgggattataggcatgcaccaccgcacctggccctacccttaatactttagtattaatgttttagtatatttgccttaaagttaatgacattctccttgctacctgtccacaatacattcataaaattcagaaaattacactgagtcattattaccatctaatcttcagatttcattaaagtttagctaattgtccatgtaatgtcctttttgtccaaagtatctaggtcagaattatgtgttgcatggagttgtcatatgtctttggtctccttcattctagaacagttctttaatctttctttgattttcgtgaccttgactcctttgaagactacagaccagttattttgtggaatcttttttagtttgaattttctcataattggattcaggttttgcatctttgtcaggtatagcagagaagtgatgctaggttcttagtgtgtcctgtcaggtagctctttatttcagttttttcctattattgattatctctttgatcacttggttaaggtagcatctgccatccttcatcactataaagctcctttttacccccacccctttttttttgagatggggttttgctcctcttgcccaggcttggagtgcaatgtctcaaaaaaaaattaatagtaatatatacagtcatagttagaagccaagatctagtttctaaatgtttgctgctgttggagtgttggtgatcccagttcctttcagtgggaaggggctaaggaatatatgggtatatttacatacatacacatatgttcactcaatctatatttatatctgtctttatctaaatatgttgaaaactatgagcttaccccaataatacaagttctaatccagtcattctagttttttccctttccatatgtataagtcctttttccaacaataataaatctgggctgggcgcagtggcttacacctgtaatcccagcactttgggaggctgaggcgggtggattgcttgaacacaggcgttctagatcagcctgggcaacatgtcaaaaccccatctatatggggaaaaaaaaaaaagttagccaggtgtggtggcacatgcctgtagtcccagctactcaggaggctgaggtgggaggatcacttgagcccaggaggtcaaggctacagcgagccgagattgcaccactgcactccagccagagctactgagtgagaccctgtctcaaatctattccctaatatatttacctaattatttaatcacccactcttcactgttgcagccctttcttcatgcagatgtcctctttactttgcttggactccaacaccctgtgttgggttggctccactctcatcccatggatgagttcactcaagctctgaccagtcacatcagggtgttctcccatatacatgcccttctcaacccgcgtgggcttcaatactctgtcctttgtcattccctgtttgggcatttcactcatcttgatctccaactctcacgtcataccacactcagatacacacagataccttctgtgctccactcaggctctgactctctactcttgtctacctagcctcaggcctccacttttgtatggatgcctaccttgctgtgtaccatgtaatggaagggaagactagtttgggggaagagaagggcagctcttcccatttaaaaagattggtttgtctttttgttttaggtttatagaattgaaatatataggtgggcttggtggctcacgcatgtaatcccagcactttgggaggctgaggcaggtggatcactttggtttaggagtttgagattagcctggtcaacatggtgaaaccccatctctactaaaaatacagaaaaattagctgggtgtggtggagggtacctgtaattccagctactcagaaggctgaggcacaagaattgcttgaacctgggaggtggaggttgcagtgagctgagatcgtgccactgctctccaacctgggcaacagagctagactccgtctcggggggaaaaaaaagaattgaaatatatgtatacacacacatatacacactctggatacatactgtatcccgagtatgtgttttgcaaatattttctcccagtctgtggcctgccttttatttcttttcttttttttgagacagggtcttcctctgtcacccacgctagagtgcggtagcacagtcacaactcaccgcagcctcaacttcctgggctcaaagtgatcctcccacctcagtctcttgagtagctgggactacagacacgtgccaccactcctggcccattatgtctgttttttgtagagatgacgtctcactatgttgcctagggtggtctcaaactcctaggctcaagtgatcatcccacctcagcctcccaaagtgctggtgagccaccatgcctggcctggcctggcctgcctttgcatttttattaaagtgtcttttgattagaagtttttttgttttgaggaagtctgttttatccacattaaaacaatatgtttttaggaacttttgcctatctattaatagtgcacaaaaatattttttggctttatatttttggctcttaggtgtgcgatttatgtcaaattaatttttatttttgagagggagtctagctttgtcacccaggctggagtgcagtggcacaatcttggctcactgcaatctccacctcccaggttcaagcaattctcctacctcagcctcctgagtagctgggattataagcacttgccaccatgcctatttttctatttttagtagagatggggttttaccatgtttgccagcctggtcttgaactcctgacctcaggtgacctgcccgcttcagcctcccaaagttgctgggattacaggcttgagtcaccatgcctggcctcaaattaatttctatgtacggttcgaggtagtcattcaggtttgcgtttttacgtgtttatcttgtttcagctttatttgttgaaatgatgttcccctctttattgaatagctttggtgcctttgtcaaaaatcagttgagtccatacataaggatatgtttttgtactctgttctgttccattaatctatctatttttataccaatattttactctcctgattttagtaagtcatgagattggatagtgttagtagtgtgaatcctctgtgttcttccttcttaaaattgcttggactgcttttggttctttgcatttatttttctttcttttttagagacaaggtcttgctctgtcggcaggctgtagtgcagtgacgcagtcctagcttactgcatcctaaaacttccgggctcaagccatcctcctgcctcaagctcccaaggcgctgggattatgtgtgtgagccaccatgcccagccttctttgcattttcatataaattttagaatcaacttgtcaatttttatcaattaaaaaaattgggattttgattaggattatgttgactttgtagggagaattaacttctcaacagcattttttttcaatccatgaacatggtataatctccagttattatcatctttaatttttcttagtgttttatagtttccaatgaggtcttgcatgccttaacatttttttctcaagtatttttatttttttaattttttaaattaatttatttttttgagatggcgttttgctcttgttgcccaggctggagtgcagtggcggaatcttggctcactgcaacctttgcctcccagtttcaagcgattcacctgcctcagcctccctagtagctgggattacaggcgggcaccaccatacctggctaattttttgtatttttagtagagacagggtttcaccatgttggccaggctgttctcaaactcttgacctcgtgatctacctgccttggccttccaaagtgctgggattataggcgtgagccaccatgcccagcctcagctatttttattttcgatgctattatacatggaatttgttttaaaaatttgtttcccagttttttgccctagcaggtatatagctgatttttcaataacttgtaattttgacataatttcagacacgattgtatttgctttataattctagctacatgttatttttttcatgtactaaagtgcagacataatgccccctaacctctaaatagttattaacaacaagtacattctctttcataaccacattgcaattatcaatgtcagaaaattaatgttgatatggtactgcttgctgtctaatgtgcatactttgagctgggcttggtggtatacacctatagtcttagctactcaggaggctgaggtgggaggatcgcttgagcctaggagttgaaggctgcagtatgccatgatcaaccctgtgaatagccactgtactccagcctgggcaacaatagtgagatccctatctttaaaaaaaaaactgcatattttactgaaattttacctattcttttctaatgccaattataaagcaaaattctggttgaggatctcatccttgatcacatggtacatttgtttgtcaaatctcattttctttaatctgaaaaagttctcacaaacattgtcttttatgacttaattttttttaaagtacagaccatttattttgtattaatagtaagttctccaatatggatttgtcttgatgcttttcattattaaagtaatgtttttgacaggaatacctcagcatgtcctcctcagtacattttatcaggagacacgatacctgtttgtctgatttctgataacgataacttcaaacacttggttaaggtggtgtctgccttatttctctcctataaatagtaccttgtagagagatacagtagtcccctttatctgctggggatgtttcaagaccagtgggtgcctgaaattacagatagtcccaagccctatatatactgtgttttttcctatgcatatgtacttttgataaattttattttattattcctttatttagtttttagaaatacggtcttacaccgttgcccagcctggtcctgaatgaacttctggcctcaagtgatcctcctgcactggtctcccaaagtgttgggactacaggtgtgagccactgtgcccagccccccaagtttaatttctaaattaggcacagtaaaagaataacaacaataactaagaataaaatataacaattgcaacaatgtactataatcaaagttatgtggatgtgatccttctttctctctcaaaatactgtaattttttggactgctgttgaccacaggttatttaaactgtggaaggtgaaacttcgggtagagggcacttactgtactttgagactatatgaatagtctcatgtactttcattcactggttttagcatccactgatgaaggattcctacctaaaacagttagtacgctgaggctgggtgcagtagctcacacctgtaatcccaacactttgggagaccgaggtgggtggatcatttgatgtcaggagtttgagaccagccttgccaacatggtgaaaccctgtctctgctaaaaatacaaaaaaattagctggacatgatggcgcatgcctgtaatctcagctactgggggagggtgaggcaggagaatcgcttgaaccctggagatggaggttgcagtgagctgagatcgtgccactgcactccagcctgggcaacagaacaagactctgtctcaaaaaaaaaaaaaaataaaaaaaataaaaaaaaattagtactctgagagttgttgtcaagtggtgattttttttttttttaagttccaccatttatctgacatctattagttgtggtttttcagtaagggggagagctttaatttattcccccatttgtttattcgttgtgaatttatggattcttttttttttttttttttttttttttgagatggagtctcatgcgctctgtcacccaggctggaatgtagtggcacaatctcggctcactgcaacctctgtctcctgggttcaagtgattcatcttccttagcttcacgagtagctggggttacaggtgcctgccaccatgcccggctaatttttgtctttttagtagagacggtttcaccatgttggccaggcttgtctctccaactcctggcctcaagcgatccacccgcctcagcctcccaaagtgctgggattacaggcataagccactgtgcccggccatggattcctttttttttttttttttttttaaaatatgaagactcgctctgttgcccaggctggagtgcagtagtgcgatcttggctcactgcagcctctgccgcctgggttcaagtgattctcctgcctcagcctcacaagtagctgggattacaggtttgtgccaccacacccagcttttatatttttagtagagatggggtttcaccatgttggccaggctggtctcgaactcctgacctcaagttatccacccgctttggcctcccaaagtgctgagatcataggcatgagccattatgcccagccctacccatggattcttattttattattgagttataatcttactatcatttttaatgtacaaattgtcgctgatttggccagtggaattgcatgtactccagcttttgtgtcctttttatgtgttttcctcattcctgaacacttctttacttacctgctttctgacccagcaagatgttctgggcttagcttatattttcccagccccacttctagaatcatccattttcctggggagctctggtttcttttagcttagaatagtatgcatgtatacatctgtacctgtttctctctttgtctgtctataggtaaatatagtattcatactaataccttgaaattagtctcttactgggggtttcattctaactttacctttctatatgtgcatcttgctactccaacattgaacaacctggcttctattatgtactttgtatctacctacttgctcagttttagtataaacacttagtagttttataattgttaacccattccactgtggaagaacaaatctaactagaactcagtatttgtttatagttcatttaggtttttagcctaaggatatatagtcaaaatacttgaagaattacttgggttagatctgtccttgtgccccttcagtgtggttatgttattcactttaagtatatgtcagtaaaaattttaaatgtatcacaaccacttcctctttttagtgctgtgggaaaacaagcactgttgcattacccttaagaaaagaaataaatttttttttttttttttttggaaacaggtctcactctgtctcccaggctggagggcaatggtgggatcgtagctcacgtcagcctcccaagtagctgggactacaggcatatgcatcctggctggctaatttttttttttttttgcagacatgggggtctcactatgttgcctaggcttatcttgaactcctgggctcaagtgatcctcccgccttgccattccaaagtgttaggattataggcgtgagccactgtccccaggtcaagaaaacaaatggatacttttttttgttgggggatttgatactattaacaaaattatatttgcatctaccttttgaccgattgtttccatttgattattcaaggcagtagtatttgtagttgcagaatattaaaagccatctaaatgctggtggtacatgagggagtggttatactttgatacatcaacatcatggagtatcatgcagctgagaagaaaatgaggaagttttcatgaaacgatagagtaatttccagaatatattgttaagtggaaaattgaagagcaaaatactctctgaggcacgcagattttgttgttgttgttgtttaaggatgaaggataaataaaacagatgtgtatattatcatttgtacaaaaagaaacacaagaagaatacaccggaaacaatgaggttggttaggggactaacaggaatggaatggagagttggagggggatggaaatggaagggatggtggggagcagtgtcacttactctgagtataaccttttatatagttttgatttgtaatcatgttatatttcacataattcaatatgtagataaaataacaaagatgggaaataaaaaacaaaatgggttacaaacaaaagatgaaacccagtgctattaacagctgtacaattcttgatgtgttcaacgtatttcattcttctttctgaatgtttgttcctggagccatcttttttcctgctctccgcatggacagtttgtggtgtaaggtggccagccatcctggtttgcccacgattgaggggtttcccggggtgtaggactttcagtgctaaaactggaaactcccaggcaaactggggcctgttggctactcttttcacgtgcatgtggctgttttcctcggactttccttatatgtaccttgggaattctcttcactttctcctggtatgtcctgcctttcctagatctcacacaaatctcatttattgttactcctttagttgtgtgcgcattctctacacaatacatttatgtagagcagattgacgtagagttgcttctagagaacaggtgcctaggaggtaaattgaggccttacatgtctttattctgtgttccacactagattgttggttgggctcagaattccagaagatgttactcttactactacattcttcagagctgcttttgagaaacatgccattgtgatttctggatatcgtgtgttttttttgaccggaagattttaggatctgtttctctcctgtgttttgagatttcatgataatgctgtggaatgggtctttttcattcattatgctgagtgttttgccttattaaactcggaaactggagtcttctcatttggggaaaatttcttgtattatttctgtcttttggctttcttcctctggaccttctatttgttggatgtgggactctcttaatggagcttctttttcttttttttttttaatcagttctctaggagataatctgttgtttttggcctctcatttttaatttctagcagattgttcatgttctttaatgttgctttatttttttaaaaaaatattctggctgagctgtagtacgtgtctgtggtcccgactactggggaggccaaggtgggagaattgcttacgaccaggagtttgagtccagcctgggcaacatagcaacaccccatatcttattttaaaaaaagcactctatgtttgttttgggcctctttgagaacatttactatgaaattttatttatctttgtttcctttcagttcttcttttgtttattgtgattatctttcatataacaatgtgcttaaatagctggtggttcttgataggtcactcaacagtgagcaaactgatggatcctgtgtttgtgagcagtgtttattggctgtgatagagggtggggacctagatgcttttaggggatcctcctgttatccatttctgaaagtgttttctcctaggctggtcactttttccaggaggagaaatcctttcatctcttgccatctacatgacagtaggctgtggttggggtcatagtactactactatttggggagctttgggagccaaggtggggagggaagctagtggtgtcatgattttctggtgttttttgtttgtttgtttgtttgtttgtttgttttgttttgtttgagatggagtttatctcttgccgcccaggctggagtgcagtggtgccatctcaactcactgcaacctctacctcctgggttcaagcaattctccagcctcagcctccctagtagctgggattacaggtgcccgccatcacccctggctaatttttgtatttttagtatagatggggtttcgccatgttggccaggctggtctcgaactcctgacctcaggtgatcttcctgcctcagcctcccaaagtgctgggattacaggtgtgagccaccatgcccagcctgcgtcatgattttctattgttggtcttatttagagtgtggcatctcactccatgccccatcccttgagctgtgcctctagtcttctgttccagaacttccgtggttcagtttaatcagaatataaatctgttttcctctggagtgggagaagggtgtattgccttactgtttagggacaggaaggagacctgagggggtctgactgttttattcagagtttgaaacagttctcctgttctcagcccctgcctctcaactcttgtctgcagtggttcctggtgcctcccacttccctgcgtgctccgggcttttgaagcactggcttgctttttgtggcttttccctctgcagccataaacttcaactttccttctttgctgagtcagttacttcttaccttcctgctcaacctggtctttcagatttctgttgacatccctcatctactgcctttttccttccccttactctttgtctttatgcatgtacacttttactgttcccttatagtcattctagtgaaccctcaggaagatgcaaagaagtatatgttcattttgccatgtttatccacaacccccaaataccattaaacaaaagaaaagaaaaaatatttaaaaaagaaaacaaaaacaaaaacaaaacaaacccttaataaatggagagcagctatgccatgttgttgattaagaagattctatatcgtaaagatgtcagttcttcccaatttgatttgtagattcaatgcagtacactacaaattttgaaaattgagatataatagttatatgtattttgggggtacaggtgatattttgatacctgtatgcaatacatagacatttttctttgtgttgggaacattaaaattcttcttttctagttattttgaaatatatagtaaattattgtttactataattttcctattctactattgaatattagaacttactccttctatttaactgtaattttataccccttaaccaacttctattttgccatgtttaaatatggaagctcaagcgtttgtacagagaaagtggaaaatgcccttggcttttatgccaaaccccaatccctctgtccccacctgccaccctggcctgttgcccctgttcttcccagtagctacaaccactatcattaatttaacgtgcatcctcctattcatatggaggtatgtggtatgtttgtttatataagtgtattttattttctttaatgtttaatagtgtaccatatcaatatagctacattttatttatccagtcccctgctatgaatatgtgcgtttttcactgttttctaatccctgttataaacactgcagcaacacttatccttaatctccatgtacaaacgtgtgagaatttctctaagggactattcccttaccttttcatattgtatgcttgggtaaaatggaagtgacttggaggctctttctgtgggggttagtgaaaagatttcattacattttctttgcattttgtaattatgatgagaaaaatataaaacttataaatcttttttaaaagttttgagaaacatgctttgttacgatcatgacatcaagttttatgcttaaaaagtctgtaattccttgtgtttaatgatgaactcttcaaattcctgatacagatctatagtcccttattaacaattctgaaatccaaaatattatgagaactgaatttttttccccttgactgatttagtagtaaaacctgacctgtgtaaactcattttgtgtcaaaactctgatgtagattgacaggtctaggttagcttatttgtttcacttggtctttgtctacattttatttttttaaaaattaatgtgtttgattatgggttgctggtggtgctgatacacaatatatgcatatattaacattctaaaattcaagaaattctaaataaaaacacatctagacccaaagatttcaggtaagggattgtggacctgtggtcaccattagctagaaagtttcagctgtgagtggcaacttacaagaatttctttctggttgatagaaactcctttcttattattagtaagaactcagacaatttctttacgttatattccaagagtgtgttcattttttaaataacagctcttccttttctttcgttgacatctataatattgaaattgcttttgataatgcaaatggatttcacattcaagagaacctttttatgttaagtattttgtttaaatggagaagccttttctcaagcatgtatggagattgctttccttgttttaattcttctttaggattttcatgagaaaattaaaacatcattaatggaccattttagccacagttttccaaagttgcattttgactttggatctttaaactttctgtatactttaataggtatgtgtaggtttaaaaaaaattttttttttttgatcagaatcttttgttttaggccaggtgtggtggctcacacctgtagtcctagcacttgaggctgaggagggtggattgcttgcgctcaggagttcaagagcagcctgggcaatatggcaagaaccccatctctacaaaaaataaaaaagttggccgggcgcggtgactcatgtcagtaatcccagcactttgggaggctgaggcaggcgaatcacgaggtcaggagttcgagactagcctggccaacatggggaaaccccgtctctactaaaaatacaaaaaattagctgggcatagtggcaggcgcctgtaaccccagctacttgggaggctgaggcaggagaattgcttgaacccgggaggcagagattgcagtgagctgagatggagccactgtactccagcccagatgacagagcaagactctgtatcaaaaaaataataataataaataaatacaataaataaataaataaattagcctggagtggtggtgcatgcctctagttccagctacttgagaggctgaggtgggaagatcacttaagcctgggaggtcaaggctgcaaatgagctgtgatagcaccgtggcactttagcctgggcccgcacagtgagaccctgtctcaaaaaacaaaaacaaaaaatacgttttagaactatcagttaaattttgtaagccattgtttacctactttaattgggatttaattcatagaccatacagttcaccaatttaaagtatacaattcagtggcttctagtatattcatgaagttatgcaaccatcaccatggtctgattgcataatatttccatcaccccaaaaggaaaccccagtaggtcattcctattctcacctcctcctagtccctagcatcaccagtctactcatttaattactaaaactttctaaaaagcaactcctctcatcttgttcttcaagagtatcttggctctttgtgaccctgtgcttttccatgtaaaattctgtaaaagatcgtgttgagagctttattggaactgcattgaatcgacaaatcaaaatgggaagaattgacatctttttttttttttttttttttttttttttttttttttttttgagacggagtcttgctctgtcacccaggctggaatgcagtggcacaatcttggcctactgtaatctccgcctcccagcttcaagcgattctcctgccttagccttgcgagtagctaggattacaggtgcacgccaccatgtctggctaattattgtatttttggtagagatggggtttcatcatgttggtcaggctggtcttgaactcctgacgtcgtgatctgcccgccttagcctctcaaagtgctgggattacaggcttgagccaccatgcccagccaaattgacatctttatgatataaaatcttcttaatcaagaacatggtatagctctccatttatttagggtttgtttgtttttgtttttttgagacagggtcttgttctgtctctcaggctagagtgcagtggtgcgatcatagctcgctgcaacctgaaactcctgggctcaagcaatgctcccacctcagcctcccgagtagctgagactacaggtgcgcgtcaccatgtgcagctaatttttgaatttttttgtagagcgggggtctccttacgttgctcatgttggtcttgaactcttgggctcaagtgatcctccttcctcagcttcccaaagtgctgggattacaagtgtagccaacatgcctggccttaccaaggttttaagcctttcttgataaagttttatgttttcttcataaaatgatgatatttcttttgtggaatttatccctgggtaacttacgttcgtttttgttataaattatatttttgttataaattatatttttgttttcttttttcttcaaccctggcaaatggatatattttctttttttttttttttgagacagagtttcgctcttgttgcccaggctggagtgcagtggcaccatgtcagctcactgcaacctccgtctcccattcaggcaattctcctgcctcagcctcctgagtggccgggattacaggcgtgcgctaccacgccgggctaatttttgtatttttactagagtcagggtttcaccatgttggccaggttggtcttgaactcctgacctcagatgatccacccacctcggcctcccgaaatgctggcattacaggcatgagcccccacacccggctgatggataaattttctaattgatgatgttaattggtgatgttagtatcacttgatttttccttttttttttttttaatgtgatctcaaatgtcacaatgtggccaaaatcttttactaggtctgagtctcttggatctttaatgtaaataattatgctatctggaaattatgacatgcttttctaatcccttcttgtttctattatgcattgaaaggaattctagtatactgttgaatggttggtgttttcttagtttttcacagccttcaagtcagccttctcctaagagatagtacttattttttgctcaggaaagatataaggaaaggcataactgatattttgcttttttttttacttcagaatctaatcttgggcagtttaaactttatcaatttaaatgttagctctcctttgctcagatttgccacattcgtagtgtcttgtattttttaaaacatctgacctgtgggagaacattttattttggtttacttattacagatggtttattttttttcaacattagcttcttttctagttaccttgccaaactagtatacgttatgcattttagaaacacttatttgtattaatagatgaacgtgagtaaaaagtatacagcaagtcttctttgtactgttttactcttgcaaaagtagaatgaaaagcttttgcagctttcctgtaaaattgaatttctttccatataaaaatgttcaaaatgtatttgtaaaaggaatacgtactcatcatcagtattaaaaaggttcagaataaagaatccctacaactacagtttttaaaagacaactcaaaaaaaaaatgggtgaaagacttgaacagatacttaaagaggaagatatgaatatggccaataagcacattaagaagtgctcatgcagccataaaaaagaatgagttcatgccctttgcagggacgtggatgaagctggaaaccatcatcctcagcaaactaacacaggaacagaaaaccaaacaccccatgttctcactcataaatgggagtgaacagtgagaacccatggacacagggagggaaacatcacacaccggggcctgttggggggtggggggaaaggggagggagagcattaggaaaaatacctaatgcatgcggggcttaaaacctagatgatgggttaataggtgcagcaaaccaccatggcacatgtatgcttatgtaacaaacctgcacattctgcacatgtatcccagaacttaaagtaaaatttaaaaaaaaaaaaacttcaaaaaaaaaaaagtgctcaacatctttagacattaggcaaattaaaatgaaaatcattgaaatctagcactctgaattaaaattaaaaccacaatgaggtaccattatactcccactagaatggctaaaataaaactgccagcttcaaataatggtgaggatttggatcaactggaactctcctcaagtgctgttgggagtataattttttttttaataaagttagctttcatccatcttatgactcggcatttctactcctagatagttattcaggagaaatgaaaacgtctccaaaaatttacaagaatgttcataatagctttattcataatagctagaacctggaaataacccaaatgtccatcaagagagaatatacaaatggttgtatatgagaatattagtctgtaataacaaataatgaactactgatgcatgcaacaatgtagatgaatagcataaacaggctgagtgaaagaagccagatacaaaagaatatatatgtatgagtccatttacatgaagaccaagaacaggcaaaagtacgttatggagatagaagtcagaacaatgattgccttcgtggggtgagaggggtggtttcttttcttttttttttttttttttttgagacggagtctggctctgtcacccaggctggagtgcagtggcgtgatctcggctcactgcaagctccgcctcccgggttcatgccattctcctgcctcagcctcccgagtagctgggactacaggcgaccgccaccactcctggctaatttttttttttttttttgtatttttagtagagacggggtttcaccgtgttagccaggatggtctcgatctcctgacctcgtgatccgcctgccttggccttccaaagtgctgggattacaggtgtgagccaccgcgcccggccgagaggggtggtttcaaatgggggttgattaggaaggggcacaaggaaacttttgggggtgttggaaatgttctatatcttgatatgcatttgttagaactcattaaatggtacacctaagatctgtgcatatcactgtgtttaaattaaactttaatttaaaaaaataggggaaggataggaggaggtatacattgatccaagggcattctcctacgtaagcacaatacagttatcacactcagaaaatttaacattaatgcagcattaccatctagtatgcagttcacattcaaatatcccagtgtcttgaaatttgctgagagtagatcttaattgtcttcacactccccatctcacactctccctctcatggtaactgtgtggtgaaggattggttaatttttttttttttttttttttttaaagacagagtcttgcactgtctcccaggctggagtgcaatggcatgatcttggctcactgcaacctttgccttccaggttcaagtgattcttgtgcctcagcctcccgagtagctgggattacaagcgtgcaccaccatgcccggctaatttttgtatttttagtagagacggggtttcaccatgttggccaggctggtctcgaactcctgaccttgtgatccacccgccttggcctcccaaagtgctgggattgcaggcatgagccaccgtgcccggcctatagaattttttatatgtaggatttagtgaaagatcacccattattaatttttttaaaagcttcatgatgaaaattattcttgtaaaatgttaaaatcttgaatcctagggcatgttttctggcacctgttattttcagtaacccatttcgtttttgtcagacatatagggttaatgtatgaaaagtcaattatgtcaaacttatagagtttatatggaaagattaatgatcttttacaaatgcagaattggctggatgtggctaattgtagaattggcctgtaacctagcacttagggaggctgaggtgggaggatcgcttgagtccaggagtttgatttttttttttaattttttattttttgagacagtctcactctgtcgccctggctgaagtgcaatggcatgatctcagctcactgcaacctccacctcccaggttcaagcgattcttgtgcctcagcttcccaagtagctgggattacaggtgtgtgccaccatgcctagctaattttttgtgtgtgtgtgtatatttagtagggggggtttcatcatgttggcccaggctggtctcgaactcccagcttcaggtggtccccccatctcggcctcccaaagtgctggattacaggtgtgggccacaatacccagcattttttatttatttatttatttatgtatttgagatggagtttcaccctgttgcccaggctggagtgcaatggcatgatcttggctcactgcagcctgcatgtcccgggttcaagcgatcctcctgccacaacctcccgagtagctgggaatacaggcatgtacaccacacccaactaatttttgtatttaagtagagtcagggtttcattatgttggtcaggctggtctcgaactctggacctcaagtgatctgcctggcttggcctcccaaaatgctggaattacaggcttgagccactgcacctggccaagcccaggactttgagaccagcctgggcaacagaccaagaccctgtctgtacaataaaaattatgcagtattagaatagcaaatcttactcttaaaagttttgttttccttgtctttttactgaaaatgcatggctttagaaacttctgggtagtttgaattgcagacaaccaggccctattcctgtttgtttaactaatgagatttgtaaacagtacttggtttccccagactctaggacctagaggaaggaaattttgatttagctcttggtaaagcatttaacacctaatttttctttttcattgttattttcttgatcacagaaaacaatacatgttaatagtagaaaatacccacaattgaaaagaatataaagcacttactaaaactctaccacctaggctaggtacggtggctcacacctgtaatcccagtactttgggaggctgaggtgggtggatcacctgaggtcaggatttcaagaccatcctggccaacatggtgaaaccccatatctactaaaactacaaaaattagctgggcgtggtggcagccacctgtaatcccagctgctcgggaggctgaggcaggaaaatcgcttgaacccaggaggaggaggttgcagtgagccgagatcatgccgctgcactccatcctgggtgacagagcgagactcttgtctcaaaaaacaaacaaaaaaactgccacctagagatggtggtggccaacagcttagtaagaatttctcttgcatttttctatgcaaacacattcaaattttcattttttaaaaaagatgatagaatcatactgtatgaatttaattgcctaattaagcttgtgtatcatcgtttctcttgtcattaaatgctgtacagctttcttttaaatggctatctgtggtagtgttccttgaaggaaataccttaaaattttttgtttttaaacttttataattgtgggaatgtaatacagtgaatgcataggacacaaatgttcagcttaaggaattattataaagtgaacaccctgtaactgccacttagatcacgacatcaaatatggccagcagccaggcgcacctcccctaccccccactgccccttcccagtcacagccccattcttccagctgaaggtaacttctgtcttgactttatgaaagttattttcttgctttcctttagattacctacccgtctttgcattcctaatgctgtagtttagttgagcttattttgaacttttcacaaacggaatcatcaactatgcattattttgtatctggctttcattcactattttgtttttaatattcatctatgtcgttagtagctatagttcattcattttttgttgctgtataatcatccatttattgaatgtactacaatttgattctgcctttggcagccactttgattgtttataatccagatttcaaatgctgcatcatgaacatgtgggttgtgatttgtgtagatgtgcatgtacttctctaaggaatccaagcctaggagggacgttgctgggtcagaggatgtttgtatcttcaactgtaccagataaggccaaattgtattctcaaatgcttggatctgttttcagtgtacttgttctactcctaattaacactacttgatattgtcaaactttaattgtagccattctgatggatgtggtagtatctcattgcggttttaatttgcattattttgcattttccttattgttagtgagattaaatacccttttatatgttttggctacttcagtatcctcttgtgcagtgcctaattcacatcttttatttttcttctgagttgccttttgcccattgatttatatttgttccccctctctccccgcccccaagaaggagtctggctctgtcacccaggctggagtacagtggcacaatcttggctcactaacctctgcctccttcgttcaagtgattctcctgcctcagcctcccaagtagctgggactgcaggtgtgcgccaccacgcccagctaatttttgtatttttagtagagacagggtttcaccatgttgaccaggcttgtcttgaacccctgacctcaagtgatccatccgccttggcctcccaaagtactgggattacaggtgcaagccaccatgcccggcctgatttataaatgtagtctaaatacaagttctttattggttatgtaagttgcagatatctttttcagctctgtaggtaataatgttttttgatgaataggaggtcttaattttaatgtagtttaattgatttgttttctcctttgtaagaatcaatatttttcatgtcctgtgtaaatctcgtattgggataaaagatacatatatattttttaggttttggctaatagttttccgttggcagcatgtgaacctttttatgccatttttactttcatcaagggtgtatgtatgattttctcatactcttgtcagaactggagatttggggtaactttgccagtgtaatagactttcttaattcattaaggagtattttatcttagtttgcccatgagagaagagagattaaatttgttttattataccaagaacatggcagtcaataagcataactaaaaatattatttagcttttgattctgaagaccatgtatggccagtaaggaaaagaggtaaagatgtagaaaactgttttcttttttttctttcttttttttttttttttttttttttgagatggagtcttgctctgtcgccaggctggagtgcagtggcgtaatctcagctcactgcatcctccacctcctgggttcaagtgattcccctgccttagcctcccgagtagctggtactacaggcgcgtgccaccacacctggctaattttttgtattttagtagaaacagggtttcaccgtgttggccaggatggtctcaatctcctgacctcgtgatccacctgcctcagcctcccaaagtgctgggattacaggtgtgagtgagccactgcacctggcctcttttttattaatttaagatacatgattttggctaaaactaatgaaaataaattgatgaaacttcaatgtagtggatcaagagactcatgaaattgatgagttagattgttgtgggcacctgttttaaatttatggtgcctttttagcagaatcatgactttatcaactctttttcacatacttttattcaaagaagacattctcctcctctgccccaacttatcttcttcttgtctccaactgccagtattctcatccacactctttgtagttttgaggtgtgagataactcccaagttcttcttgggaatgggtagagtttaattctgagtctacatgtcgtgagtctattggaagaagcttttcttaggaggtgaaatttgggatggagttctcctctacactctagccctgtagccacaatctttggtttagatctttggcattttttgttggacatctttcatagtgtctacctcttcttcctgcctctaggcttcccgcggttgccagatgaaattccgaaaactcaaatcattcctgtagttaatattcttcagtgactccggaacaatcaaattcttgatccctccacatgggtcttaccatctgccctcgtgccctctctagcttcctgcatattgagttccccacagccctagtttccctgagtttggaatattctttcacatttgctcatcccactatctagaaacttcctacaatctctctactgccaacctgtctgtctagttgctaatttattttaaaacccagcttaagcaacacatccttttggatgtgttacttccctgatcgttattcacctccatgccgtctttcgctcaactcattgaagttaggtgcccctcttctgggctgccacggctctccatgttcacctatgtactgtactttgtattgtaatcagtttaattgacccccctacttatttgagctccttgagagcagggccttgattcctgctccttagcactccaccctccccagaactaacatggtatagcttataggaggcaccctataaatgtttaatgaatgtttgaatgcaaaagtgtatctaactttcttaatgatatatttatgaaaagaacttgctgagaaaaaggtcagttagaaagggttaggaacttgtcagtattctaaatccctctttttcaaaagcatacacatttaggaatgactgccttgctttaagaatttgtagggttttgttcacctgagttgtaccttgctgtcacagaaggatgccctgcagtggtctcgccatccagctacagtggagggagaggagccagaggacacggctgatgtggagagctgtgggtctaatgaagccagcactgtgagtggtgaaaacgatggtaaggaccctttaatggatgggtgagggagccacagcaggcactagggactaacctttattttcctctcttccagtatctcttgatgaaacatcttcgaacgcatcctgttctacagaatctcagagtcgacctctttccaatcccagggacagctacagagcttcctcacaggtaaggaagaggtagagcctagcctgggaggatgaatgatgacaggtataaggcaagatccctccttctcctgtagtgagctgtgaacatgttcagagagtgaagaatagaagtctggtctccagctgttagtagcatttaacagggggccgcaggaaaagtgaacagtaggtttgagcagagtaagttttttctctttctttaccttggctgagtgattttgttcattctttttttttttttttttttttttttttgagatggagtcgcgcctgtcgctcaggctggagtgcagtggcatgatctccgcttactgcaacctctgcctctggggttcaagcaattctcctgcctcagcctcccgagtagctgggattacaggcacgtaccaccgtgcctggctaatatttgtatttttagtagagatagggttttgccatgttggccaggctagtctcgaattcctgacctcaagtggtctgtccacctcagcctcccaaagtgctgggattacaggtgtgagtcaccatgcctagcctgattctgttcattcttgtaatatacacgtgaacattcagggagaagtgtggttagtgcagagtagagagaacaggagattgggagtgagcagtttgaatgataaccctgcacttactagccttgagacttggggaaaattccttacctgtaaaatggggataacagtacatttttgtagcttggaatgatgcttggcacagtgaccagcacgtagctcagtaacattaaccagtgctcttgtcagcattatttgacagatctggttgaagacgaacttcattttacaagagcgtgagtagagatagtgtcgccagggaatgcttttgtggctctgcagttgacttgggctctcttttgttctctcttggaacgcaggcgaacaaacaaaagaaaaagactggggtgatgctgcctcgagttgtcctgactcctctgaaggtaaacggggcccacgtggaatctgcatcaggtatgtgtaaactcatggttgtgatgctttttcctcaggtcctggggacctggcctccctctgtcagcagtttctgacctaatagtgcgatactaggagagctgtcgggccacaggcaagagccctgccaatggatgaggcctgccataggttctagtgctgggctctgctgtgtgccttcctctttgtctcccagggcagtcgtgaggatccaacggaggatttgattatgccagtgctttgtaaaaggtgtagtgctatacaaataaagatggcagtttggcacctgtaaggtgatattttaaagccagaccatgaagtggtggtttctctcagcctaaggctgggagatggaagcatcccagtattgctggcagcatctcagggagagctgggagaaatgagcttgtctgagagccatgggcgcggcttggtgatacttttgaccagtggaatgctgtgccttcagggttctcgggctgccacgccgatggcgagagcggcagcccgtccagcagcagcagcggctctctggccctgggcagcgctgctattcgtggccaggccgaggtcacccaggaccctgccccgctcctgagaggcttccggaagccagccacaggtgagtggcgtggcacttatttctctgcctgtaaagggggcccctgcaggcctagtgagaagatgatgtcttctattccaattcctttaccagtttatttttacttcttgggtggccctctattttttgtttatttttactttgtaatttgcttatttcatttgtagctctctgctaaactgtgagaactcctttcaggcagagatgatgccatactgtttctcgtatgtccctgtaggcctagtctatgcctgccttgtactgcatagtaagtgtttgagtgagtgaaaactgcttctgagcttctcagtgaagaattccaacaccccttttctctcttcggtagcatgttgccacatttgttcccaacatctgtagtctgtatcagacgctcccttttttacaacctagtcagtatgttgagtggaagcatgtgttgaattttggggatgcaaacaaattcagtagtgctattatctatacctcccacccctcaccccaccccacccccattagggttatttgcaatattagtatgctctttgctatttcaggatttaattcttaatgacccacatgctggttttttttgtagatgaaaccattaagaagacaagcttttgcttctgagaattagagtaaacctttgcccatgtgagaactaggtttgctgtcttgctgcttttccagctcatcagcagtgctcaatctaaggtctcacaggtctctagtcctagtgagtttttcagataaaagtaacatttgctgaggcttctgtgagaggtattagcatctcagttggctttgaacagtccttagtacatttctgcatggagatcatccctctccagcccattcacagaaggcttctgcagatttttctatagtatactaactagatttacttttagataattctcatgatttctccagttgagctttgtggagcctgttctccagctttggagggaagctcagaacctgccccttgcctgattttctgtgggaagtgtccttttcaggctgtgacacagcacggtacagagttttgtctgaggagatgcaaccccaagtgttctcctgggtttgatggcaatgacagtaacctggaaagaatatacaggttaggatggactggacaattgagcagattgtgtgcagataactcctgggtagcttgggtgctgtcaccaaagttttcccatcagttggcatttgttttttcttttaaaaagctgaaatctataccttgcttcaaaaatcataggtcaaatgaagcgcaacagaggggaagaaatagattttgagacacctgggtccattcttgtcaacaccaacctccgtgccctgatcaactctcggaccttccatgccttaccatcacacttccagcagcagctcctcttcctcctgcctgaagtagacagacaggtgcacatgggcagcctcccctttgcctctctctgggtgggcttctgttctcttttaagtttatttattaggatttttttcccccttgatccttctaggtggggacggatggcctgttgcgtctcagcagcagtgcactaaataacgagttttttacccatgcggctcagagctggcgggagcgcctggctgatggtatgtagacttggtcatctcggacggcttgcgacgcacctgtcgtgtggtgttgcatgtctcctggtatttaaaacccaagctcgctcttctcaaagggttatttagtataagacaggctttcctcattactaataaatgtggtgggtattaatatgccacacagctctgaagaaataacagggtgtttaggtttagactttcactatattgtatgtatgtactcattcatcatctcctttttatcattaaaactataaacacctattccagtaatcatggcatggtctggaagggtggaggaaagtacactgtggagttgagaagctccaccatgctctgtctttttaggcattgtcacttggagtgattgttagcttctattaatttaattttgtggggaaagtgttgagagggctctaagaacacccctaggtttgatgatttgctagcaggactcagcatatagttatatgtacagggctgcggtttattacagcagaagaatacaaagtaaaatcagcagatggagaggcacatggggtggggtccaggggaaaccatgtgttggcttccaaggtcctctctcagtggtgtcacacaggacacatttaattcccccagtaacaagttatgataacatgtatgaaatgtctactagggaaacctgttaaagactcggtgcccagggtttttatcaagggctgatcatgtaggaagccactgcttggcacatacccagcttccagattcccaaaaggaaggcaggtgttcagcataagccgtattgtttgcataaacaatttaggcacagtgaacaactcatatcagttagggtggttgggaaccctcctgaaatctaagttcccagatgtcagccaagggctgttcttttaagtaggcctttctaagagtaaacaatcaggcctacctactatgttaactttttatctacacaggaaatatgaatggttcttttgtttgttgttttgctttttcgatcaaggagttgcttggtctcactttaaggaagagtgaatttcccttatagtagatgtgttagctctgtccctataagagcatgatgtgagagagcctttagaagagacgtgttgttttaaaactgggagattcagctgtccataagacagacattaatatcccgaatgcacttactagaagaggtttatttctccctaggtgaatttactcatgagatgcaagtcaggatacgacaggaaatggagaaggaaaagaaggtggaacaatggaaagaaaagttctttgaagactactatggacagaagtaaggcagttggagctatgagtcctggtctggggttttgaggggatagaggtagatggtctcaaaatagcatatacttatgtgttggacaagaatgtggagattgcagtgacacttgggtcatttatcttaatgccattcatagtgaagctaacagaagtttttccatggttagattgtgcaccacacagatttattttgttctgagatatctgtgtttctgggtccatattattcatagaaataagagacatgtccactctggcctgaaactgatggctgtgattttgatttgcaggctgggtttgaccaaagaagagtcattgcagcagaacgtgggccaggaggaggctgaaatcaaaagtggcttgtgtgtcccaggagaatcagtgcgtatacagcgtggtccagccacccgacagcgagatgggcattttaagaaacgctctcggccagatctccgaaccagagccagaaggaatctgtacaaaaaacaggagtcagaacaagcaggggttgctaaggatgcaaaatctgtggcctcagatgttcccctctacaaggatggggaggctaagactgacccagcagggctgagcagtccccatctgccaggcacatcctctgcagcacccgacctggagggtcccgaattcccagttgagtctgtggcttctcggatccaggctgagccagacaacttggcacgtgcctctgcatctccagacagaattcctagcctgcctcaggaaactgtggatcaggaacccaaggatcagaagaggaaatcctttgagcaggcggcctctgcatcctttcccgaaaagaagccccggcttgaagatcgtcagtcctttcgtaacacaattgaaagtgttcacaccgaaaagccacagcccactaaagaggagcccaaagtcccgcccatccgggtaggagactgtttgattcctggctgccctggagccaggttttctttgagggtcatgagattatagagcccttaactctggggccctgaagttaattatgtgggaatgtagagctttttatgagaggtttgcttgagacagccttcaagtttagtgagataggttcttttcttcctcacttttttgttcactctgttgaagttatcttgagaggtcaaaatcctttggggttttaaaatctttttaagatagcattagattctttgctatagtgactcacacagtcccaccagaaattaaatttagaagtgtggcatataacagcccttgagcagaatcttctctgaatggtgtgttatggcgccattttactatgatgatttcattgttttaaggaaattatttgattctgtatgccatgacccttaagctactagaatcctagttttgctttacagtccctaggtcagatcacccagtcagttaaaactattttctaattctttttttgcagattcaactttcacgtatcaaaccaccctgggtggttaaaggtcagcccacttaccagatatgcccccggatcatccccaccacggagtcctcctgccggggttggactggcgccaggaccctcgcagacattaaagcccgtgctctgcaggtccgaggggcgagaggtcaccactgccatagagaggcggccaccactgccatcggaggggggggtggcccgggtggaggtggcggcggggccaccgatgagggaggtggcagaggcagcagcagtggtgatggtggtgaggcctgtggccaccctgagcccaggggaggcccgagcacccctggaaagtgtacgtcagatctacagcgaacacaactactgccgccttatcctctaaatggggagcatacccaggccggaactgccatgtccagagctaggagagaggacctgccttctctgagaaaggaggaaagctgcctactacagagggctacagttggactcacagatgggctaggagatgcctcccaactccccgttgctcccactggggaccagccatgccaggccttgcccctactgtcctcccaaacctcagtagctgagagattagtggagcagcctcagttgcatccggatgttagaactgaatgtgagtctggcaccacttcctgggaaagtgatgatgaggagcaaggacccaccgttcctgcagacaatggtcccattccgtctctagtgggagatgatacattagagaaaggaactggccaagctcttgacagtcatcccactatgaaggatcctgtaaatgtgacccccagttccacacctgaatcctcaccgactgattgcctgcagaacagagcatttgatgacgaattagggcttggtggctcatgccctcctatgagggaaagtgatactagacaagaaaacttgaaaaccaaggctctcgtttctaacagttctttgcattggatacccatcccatcgaatgatgaggtagtgaaacagcccaaaccagaatccagagaacacataccatctgttgagccccaggttggagaggagtgggagaaagctgctcccacccctcctgcattgcctggggatttgacagctgaggagggtctagatcctcttgacagccttacttcactctggactgtgccatctcgaggaggcagtgacagcaatggcagttactgtcaacaggtggacattgaaaagctgaaaatcaacggagactctgaagcactgagtcctcacggtgagtccacggatacagcctctgactttgaaggtcacctcacggaggacagcagtgaggctgacactagagaagctgcagtgacaaagggatcttcggtggacaaggatgagaaacccaattggaaccaatctgccccactgtccaaggtgaatggtgacatgcgtctggttacaaggacagatgggatggttgctcctcagagctgggtgtctcgagtatgtgcggtccgccaaaagatcccagattccctactgctggccagtactgagtaccagccaagagccgtgtgcctgtccatgcctgggtcctcagtggaggccactaacccacttgtgatgcagttgctgcagggtagcttgcccctagagaaggttcttccaccagcccacgatgacagcatgtcagaatccccacaagtaccacttacaaaagaccagagccatggctcgctacgcatgggatctttacatggtcttggaaaaaacagtggcatggttgatggaagcagccccagttctttaagggctttgaaggagcctcttctgccagatagctgtgaaacaggcactggtcttgccaggattgaggccacccaggctcctggagcaccccaaaagaattgcaaggcagtcccaagttttgactccctccatccagtgacaaatcccattacatcctctaggaaactggaagaaatggattccaaagagcagttctcttcctttagttgtgaagatcagaaggaagtccgtgctatgtcacaggacagtaattcaaatgctgctccaggaaagagcccaggagatcttactacctcgagaacacctcgtttctcatctccaaatgtgatctcctttggtccagagcagacaggtcgggccctgggtgatcagagcaatgttacaggccaagggaagaagctttttggctctgggaatgtggctgcaacccttcagcgccccaggcctgcggacccgatgcctcttcctgctgagatccctccagtttttcccagtgggaagttgggaccaagcacaaactccatgtctggtggggtacagactccaagggaagactgggctccaaagccacatgcctttgttggcagcgtcaagaatgagaagacttttgtggggggtcctcttaaggcaaatgccgagaacaggaaagctactgggcatagtcccctggaactggtgggtcacttggaagggatgccctttgtcatggacttgcccttctggaaattaccccgagagccagggaaggggctcagtgagcctctggagccttcttctctcccctcccaactcagcatcaagcaggcattttatgggaagctttctaaactccaactgagttccaccagctttaattattcctctagctctcccacctttcccaaaggccttgctggaagtgtggtgcagctgagccacaaagcaaactttggtgcgagccacagtgcatcactttccttgcaaatgttcactgacagcagcacggtggaaagcatctcgctccagtgtgcgtgcagcctgaaagccatgatcatgtgccaaggctgcggtgcgttctgtcacgatgactgtattggaccctcaaagctctgtgtattgtgccttgtggtgagataataaattatggccatgggaaacattgtatatttagtgtgtgtattttgataatgattgatcttaaatctgtatacagaatatcattgatataatactctttaggcaggagcactcttgccttcccccaaaatttacactgctaaagccctctgtcacttggcgacccttctggtcttgctggaggggtttcctgggtataacccattgggctgcccaaggccagccagcctgagctctcctgcaagacagagcctgatgtggcacggagtggggttgcggggggtggggggactgcctgactcccagagggacttgaaactgaagcaagaaggttgcattctccaccaagggagttaacctacctgaactaagtagaaatgccagtcttccactaccccctccctgccatcttttcttctgctactttggggagttgatggccaggaaagaagccagcacagggttaaagtaactcctggcattgcccaccagggggctggtgcacctgctgacctcagggtcacagttgagtcatttgccagttgacggagcaagtttgaccttggttctgttgctgaagcaaatttggaacttttctgtctcagtgtgatccactaacccacaggatcatttggaaccttgaatagctctgcttggacaatggggttggggaatagggttgtctttcctatgaaaatgccatctgtagaccttgtgagtcagccgtccagatgtttgcaggtgaattcctctgcttgacatcctccctgtcactttggaccctatgggagtgggcatctccacgcacctgtgtatgtgaaagtcattttacatttcaaagcagtgtgtgtttcttatttttatatttttaactctttattcttggatgtataaagtgaactttttggcttctgtaagtatgctctatgcacctctaatgttttatcatgtatttatatgttgtacacagtactggctgattctgtaaatggatgtattgtacagagaacatgaacgtctcttcctaattttacatcttcagcatcattgcattaaagtggtgtaatctccttctctacatctgttgtcagagccactgagtgctgtgctgctcgacgtgagggtgaaatgattgacttgtgacctgccaggttgcccgatgccctgttgggtcaccggctggacctgctgcagcctgcagagccacagtcagcctgcccacatgccaccgagcaaacgcatcttgcttttcacatctctcctcctacagccttaatggctgcttgctgccatatgtgacaaatcaccaccaccagtgttaagtgcttctggattcatgggtgagttccctgggcagcccccaggaaggccttccagatctggctccagggtcaccacctgtcacagcaatacctgggaccatgctctcctgggactgtgaggctccttttgacgtacttttgacatcaggcaggtttgggaagaaacaaagccatgcctgctcctgcctctctcccaacatgtttccagcaagtagatgcccctgtgtgtgttttcccttgccttgtttcctgccttatatcttgtatttcgacttattacagagttgagggttcttgcttaatttagatcaagtataaaatttgtatgacttcaagtctcattttatctgaaaggtttttttctcatttaatctgatgtggcattttcgtcatctgaagcatgagtgacaagttgggaatgatgtggtgatttagaatgcagtattggccaagtccaagttgtcaacttaagcgtctgtttaccaaagaccgggaacaggggcccaaacatgtccagtcctcttcttccctctgctggaacctttggggacactcaagggtacagtttgacactgatctggtccatgaggctgcccagagaaagcactgcttctgtatgtctcttgtggtattggaacaataaacccgtacaacctgca

SEQ ID NO: 24 - RNA, 7052 nt, Homo sapiens
  source 1..7052: mol_type = mRNA
acacacactcacacacacccacggcagacacgcacgcacccgggcgccgaagggaaagccgcgtctcgccctcccgccccgccgtcggtcctgtctcagtccctcagcagagcgggaaagcggaggccggagccgtgacctctgaccccgtggttatgcggagccgccgcattccttagcgatcgcggggcagccgccgctgccgccgtgggcgactgacgcagcgcgggcgcgtggagccgccgccgcccctcccccaccgccgctctcgcgccagccggtccccgcgtgcccgccccttctccccggccgcacccgagacctcgcgcgccgccgctgccacgcgccccccccaccgccgccgccgccccagccccgcgccaccgccccagcccgcccagcccggaggtcccgcgtggagctgccgccgccgccggggagaaggatgaaggacaaacagaagaagaagaaggagcgcacgtgggccgaggccgcgcgcctggtattagaaaactactcggatgctccaatgacaccaaaacagattctgcaggtcatagaggcagaaggactaaaggaaatgagaagtgggacttcccctctcgcatgcctcaatgctatgctacattccaattcaagaggaggagaggggttgttttataaactgcctggccgaatcagccttttcacgctcaagaaggatgccctgcagtggtctcgccatccagctacagtggagggagaggagccagaggacacggctgatgtggagagctgtgggtctaatgaagccagcactgtgagtggtgaaaacgatgtatctcttgatgaaacatcttcgaacgcatcctgttctacagaatctcagagtcgacctctttccaatcccagggacagctacagagcttcctcacaggcgaacaaacaaaagaaaaagactggggtgatgctgcctcgagttgtcctgactcctctgaaggtaaacggggcccacgtggaatctgcatcagggttctcgggctgccacgccgatggcgagagcggcagcccgtccagcagcagcagcggctctctggccctgggcagcgctgctattcgtggccaggccgaggtcacccaggaccctgccccgctcctgagaggcttccggaagccagccacaggtcaaatgaagcgcaacagaggggaagaaatagattttgagacacctgggtccattcttgtcaacaccaacctccgtgccctgatcaactctcggaccttccatgccttaccatcacacttccagcagcagctcctcttcctcctgcctgaagtagacagacaggtggggacggatggcctgttgcgtctcagcagcagtgcactaaataacgagttttttacccatgcggctcagagctggcgggagcgcctggctgatggtgaatttactcatgagatgcaagtcaggatacgacaggaaatggagaaggaaaagaaggtggaacaatggaaagaaaagttctttgaagactactatggacagaagctgggtttgaccaaagaagagtcattgcagcagaacgtgggccaggaggaggctgaaatcaaaagtggcttgtgtgtcccaggagaatcagtgcgtatacagcgtggtccagccacccgacagcgagatgggcattttaagaaacgctctcggccagatctccgaaccagagccagaaggaatctgtacaaaaaacaggagtcagaacaagcaggggttgctaaggatgcaaaatctgtggcctcagatgttcccctctacaaggatggggaggctaagactgacccagcagggctgagcagtccccatctgccaggcacatcctctgcagcacccgacctggagggtcccgaattcccagttgagtctgtggcttctcggatccaggctgagccagacaacttggcacgtgcctctgcatctccagacagaattcctagcctgcctcaggaaactgtggatcaggaacccaaggatcagaagaggaaatcctttgagcaggcggcctctgcatcctttcccgaaaagaagccccggcttgaagatcgtcagtcctttcgtaacacaattgaaagtgttcacaccgaaaagccacagcccactaaagaggagcccaaagtcccgcccatccggattcaactttcacgtatcaaaccaccctgggtggttaaaggtcagcccacttaccagatatgcccccggatcatccccaccacggagtcctcctgccggggttggactggcgccaggaccctcgcagacattaaagcccgtgctctgcaggtccgaggggcgagaggtcaccactgccatagagaggcggccaccactgccatcggaggggggggtggcccgggtggaggtggcggcggggccaccgatgagggaggtggcagaggcagcagcagtggtgatggtggtgaggcctgtggccaccctgagcccaggggaggcccgagcacccctggaaagtgtacgtcagatctacagcgaacacaactactgccgccttatcctctaaatggggagcatacccaggccggaactgccatgtccagagctaggagagaggacctgccttctctgagaaaggaggaaagctgcctactacagagggctacagttggactcacagatgggctaggagatgcctcccaactccccgttgctcccactggggaccagccatgccaggccttgcccctactgtcctcccaaacctcagtagctgagagattagtggagcagcctcagttgcatccggatgttagaactgaatgtgagtctggcaccacttcctgggaaagtgatgatgaggagcaaggacccaccgttcctgcagacaatggtcccattccgtctctagtgggagatgatacattagagaaaggaactggccaagctcttgacagtcatcccactatgaaggatcctgtaaatgtgacccccagttccacacctgaatcctcaccgactgattgcctgcagaacagagcatttgatgacgaattagggcttggtggctcatgccctcctatgagggaaagtgatactagacaagaaaacttgaaaaccaaggctctcgtttctaacagttctttgcattggatacccatcccatcgaatgatgaggtagtgaaacagcccaaaccagaatccagagaacacataccatctgttgagccccaggttggagaggagtgggagaaagctgctcccacccctcctgcattgcctggggatttgacagctgaggagggtctagatcctcttgacagccttacttcactctggactgtgccatctcgaggaggcagtgacagcaatggcagttactgtcaacaggtggacattgaaaagctgaaaatcaacggagactctgaagcactgagtcctcacggtgagtccacggatacagcctctgactttgaaggtcacctcacggaggacagcagtgaggctgacactagagaagctgcagtgacaaagggatcttcggtggacaaggatgagaaacccaattggaaccaatctgccccactgtccaaggtgaatggtgacatgcgtctggttacaaggacagatgggatggttgctcctcagagctgggtgtctcgagtatgtgcggtccgccaaaagatcccagattccctactgctggccagtactgagtaccagccaagagccgtgtgcctgtccatgcctgggtcctcagtggaggccactaacccacttgtgatgcagttgctgcagggtagcttgcccctagagaaggttcttccaccagcccacgatgacagcatgtcagaatccccacaagtaccacttacaaaagaccagagccatggctcgctacgcatgggatctttacatggtcttggaaaaaacagtggcatggttgatggaagcagccccagttctttaagggctttgaaggagcctcttctgccagatagctgtgaaacaggcactggtcttgccaggattgaggccacccaggctcctggagcaccccaaaagaattgcaaggcagtcccaagttttgactccctccatccagtgacaaatcccattacatcctctaggaaactggaagaaatggattccaaagagcagttctcttcctttagttgtgaagatcagaaggaagtccgtgctatgtcacaggacagtaattcaaatgctgctccaggaaagagcccaggagatcttactacctcgagaacacctcgtttctcatctccaaatgtgatctcctttggtccagagcagacaggtcgggccctgggtgatcagagcaatgttacaggccaagggaagaagctttttggctctgggaatgtggctgcaacccttcagcgccccaggcctgcggacccgatgcctcttcctgctgagatccctccagtttttcccagtgggaagttgggaccaagcacaaactccatgtctggtggggtacagactccaagggaagactgggctccaaagccacatgcctttgttggcagcgtcaagaatgagaagacttttgtggggggtcctcttaaggcaaatgccgagaacaggaaagctactgggcatagtcccctggaactggtgggtcacttggaagggatgccctttgtcatggacttgcccttctggaaattaccccgagagccagggaaggggctcagtgagcctctggagccttcttctctcccctcccaactcagcatcaagcaggcattttatgggaagctttctaaactccaactgagttccaccagctttaattattcctctagctctcccacctttcccaaaggccttgctggaagtgtggtgcagctgagccacaaagcaaactttggtgcgagccacagtgcatcactttccttgcaaatgttcactgacagcagcacggtggaaagcatctcgctccagtgtgcgtgcagcctgaaagccatgatcatgtgccaaggctgcggtgcgttctgtcacgatgactgtattggaccctcaaagctctgtgtattgtgccttgtggtgagataataaattatggccatgggaaacattgtatatttagtgtgtgtattttgataatgattgatcttaaatctgtatacagaatatcattgatataatactctttaggcaggagcactcttgccttcccccaaaatttacactgctaaagccctctgtcacttggcgacccttctggtcttgctggaggggtttcctgggtataacccattgggctgcccaaggccagccagcctgagctctcctgcaagacagagcctgatgtggcacggagtggggttgcggggggtggggggactgcctgactcccagagggacttgaaactgaagcaagaaggttgcattctccaccaagggagttaacctacctgaactaagtagaaatgccagtcttccactaccccctccctgccatcttttcttctgctactttggggagttgatggccaggaaagaagccagcacagggttaaagtaactcctggcattgcccaccagggggctggtgcacctgctgacctcagggtcacagttgagtcatttgccagttgacggagcaagtttgaccttggttctgttgctgaagcaaatttggaacttttctgtctcagtgtgatccactaacccacaggatcatttggaaccttgaatagctctgcttggacaatggggttggggaatagggttgtctttcctatgaaaatgccatctgtagaccttgtgagtcagccgtccagatgtttgcaggtgaattcctctgcttgacatcctccctgtcactttggaccctatgggagtgggcatctccacgcacctgtgtatgtgaaagtcattttacatttcaaagcagtgtgtgtttcttatttttatatttttaactctttattcttggatgtataaagtgaactttttggcttctgtaagtatgctctatgcacctctaatgttttatcatgtatttatatgttgtacacagtactggctgattctgtaaatggatgtattgtacagagaacatgaacgtctcttcctaattttacatcttcagcatcattgcattaaagtggtgtaatctccttctctacatctgttgtcagagccactgagtgctgtgctgctcgacgtgagggtgaaatgattgacttgtgacctgccaggttgcccgatgccctgttgggtcaccggctggacctgctgcagcctgcagagccacagtcagcctgcccacatgccaccgagcaaacgcatcttgcttttcacatctctcctcctacagccttaatggctgcttgctgccatatgtgacaaatcaccaccaccagtgttaagtgcttctggattcatgggtgagttccctgggcagcccccaggaaggccttccagatctggctccagggtcaccacctgtcacagcaatacctgggaccatgctctcctgggactgtgaggctccttttgacgtacttttgacatcaggcaggtttgggaagaaacaaagccatgcctgctcctgcctctctcccaacatgtttccagcaagtagatgcccctgtgtgtgttttcccttgccttgtttcctgccttatatcttgtatttcgacttattacagagttgagggttcttgcttaatttagatcaagtataaaatttgtatgacttcaagtctcattttatctgaaaggtttttttctcatttaatctgatgtggcattttcgtcatctgaagcatgagtgacaagttgggaatgatgtggtgatttagaatgcagtattggccaagtccaagttgtcaacttaagcgtctgtttaccaaagaccgggaacaggggcccaaacatgtccagtcctcttcttccctctgctggaacctttggggacactcaagggtacagtttgacactgatctggtccatgaggctgcccagagaaagcactgcttctgtatgtctcttgtggtattggaacaataaacccgtacaacctgca

SEQ ID NO: 25 - RNA, 1084 nt, Homo sapiens
  source 1..1084: mol_type = mRNA
cacacccacggcagacacgcacgcacccgggcgccgaagggaaagccgcgtctcgccctcccgccccgccgtcggtcctgtctcagtccctcagcagagcgggaaagcggaggccggagccgtgacctctgaccccgtggttatgcggagccgccgcattccttagcgatcgcggggcagccgccgctgccgccgtgggcgactgacgcagcgcgggcgcgtggagccgccgccgcccctcccccaccgccgctctcgcgccagccggtccccgcgtgcccgccccttctccccggccgcacccgagacctcgcgcgccgccgctgccacgcgccccccccaccgccgccgccgccccagccccgcgccaccgccccagcccgcccagcccggaggtcccgcgtggagctgccgccgccgccggggagaaggatgaaggacaaacagaagaagaagaaggagcgcacgtgggccgaggccgcgcgcctggtattagaaaactactcggatgctccaatgacaccaaaacagattctgcaggtcatagaggcagaaggactaaaggaaatgagaagtgggacttcccctctcgcatgcctcaatgctatgctacattccaattcaagaggaggagaggggttgttttataaactgcctggccgaatcagccttttcacgctcaaggtgtgagccactgcaccaggccccttcatcttaattttaatatatctttgaataaacaccattgtatgaacctgctgtaagcttgggagtggtctgttagtctacagcttgtgtctgagatgtgctaattgaatatttgctcagtacctcatcttaactgcctttggctttatgttgcttatccttcatagtatcttgttcattggccttttacatccataggcatcacttctctgatattcgttgtgctcttttaatggattaatggtttgcttggttggttcctctagttagactgtaaactccttgagagcagagtctgtattttattaattacccacagtactaggtacatagttgccttcaataaatatatatttaatgaaaaaaaaaaaaaaaa

SEQ ID NO: 26 - RNA, 6667 nt, Homo sapiens
  source 1..6667: mol_type = mRNA
aaattccggcggaggggcgagctggcaggccggctcctcccactctgggcagcggggtcccgcgtcccctcccccactatttggcagcgtctgggggtctggggcagcttcgttcattcacccgggggagttgggtttccgggaagggtcggaagctcctccctcgcttcctggtgggtaatggggtggtgcctttgactccgggggtggaaaagcgaccccacattcaaggacgccaatggcatgttgagctttcccaatctaaaccaggtattagaaaactactcggatgctccaatgacaccaaaacagattctgcaggtcatagaggcagaaggactaaaggaaatgagaagtgggacttcccctctcgcatgcctcaatgctatgctacattccaattcaagaggaggagaggggttgttttataaactgcctggccgaatcagccttttcacgctcaagaaggatgccctgcagtggtctcgccatccagctacagtggagggagaggagccagaggacacggctgatgtggagagctgtgggtctaatgaagccagcactgtgagtggtgaaaacgatgtatctcttgatgaaacatcttcgaacgcatcctgttctacagaatctcagagtcgacctctttccaatcccagggacagctacagagcttcctcacaggcgaacaaacaaaagaaaaagactggggtgatgctgcctcgagttgtcctgactcctctgaaggtaaacggggcccacgtggaatctgcatcaggtcaaatgaagcgcaacagaggggaagaaatagattttgagacacctgggtccattcttgtcaacaccaacctccgtgccctgatcaactctcggaccttccatgccttaccatcacacttccagcagcagctcctcttcctcctgcctgaagtagacagacaggtggggacggatggcctgttgcgtctcagcagcagtgcactaaataacgagttttttacccatgcggctcagagctggcgggagcgcctggctgatggtgaatttactcatgagatgcaagtcaggatacgacaggaaatggagaaggaaaagaaggtggaacaatggaaagaaaagttctttgaagactactatggacagaagctgggtttgaccaaagaagagtcattgcagcagaacgtgggccaggaggaggctgaaatcaaaagtggcttgtgtgtcccaggagaatcagtgcgtatacagcgtggtccagccacccgacagcgagatgggcattttaagaaacgctctcggccagatctccgaaccagagccagaaggaatctgtacaaaaaacaggagtcagaacaagcaggggttgctaaggatgcaaaatctgtggcctcagatgttcccctctacaaggatggggaggctaagactgacccagcagggctgagcagtccccatctgccaggcacatcctctgcagcacccgacctggagggtcccgaattcccagttgagtctgtggcttctcggatccaggctgagccagacaacttggcacgtgcctctgcatctccagacagaattcctagcctgcctcaggaaactgtggatcaggaacccaaggatcagaagaggaaatcctttgagcaggcggcctctgcatcctttcccgaaaagaagccccggcttgaagatcgtcagtcctttcgtaacacaattgaaagtgttcacaccgaaaagccacagcccactaaagaggagcccaaagtcccgcccatccggattcaactttcacgtatcaaaccaccctgggtggttaaaggtcagcccacttaccagatatgcccccggatcatccccaccacggagtcctcctgccggggttggactggcgccaggaccctcgcagacattaaagcccgtgctctgcaggtccgaggggcgagaggtcaccactgccatagagaggcggccaccactgccatcggaggggggggtggcccgggtggaggtggcggcggggccaccgatgagggaggtggcagaggcagcagcagtggtgatggtggtgaggcctgtggccaccctgagcccaggggaggcccgagcacccctggaaagtgtacgtcagatctacagcgaacacaactactgccgccttatcctctaaatggggagcatacccaggccggaactgccatgtccagagctaggagagaggacctgccttctctgagaaaggaggaaagctgcctactacagagggctacagttggactcacagatgggctaggagatgcctcccaactccccgttgctcccactggggaccagccatgccaggccttgcccctactgtcctcccaaacctcagtagctgagagattagtggagcagcctcagttgcatccggatgttagaactgaatgtgagtctggcaccacttcctgggaaagtgatgatgaggagcaaggacccaccgttcctgcagacaatggtcccattccgtctctagtgggagatgatacattagagaaaggaactggccaagctcttgacagtcatcccactatgaaggatcctgtaaatgtgacccccagttccacacctgaatcctcaccgactgattgcctgcagaacagagcatttgatgacgaattagggcttggtggctcatgccctcctatgagggaaagtgatactagacaagaaaacttgaaaaccaaggctctcgtttctaacagttctttgcattggatacccatcccatcgaatgatgaggtagtgaaacagcccaaaccagaatccagagaacacataccatctgttgagccccaggttggagaggagtgggagaaagctgctcccacccctcctgcattgcctggggatttgacagctgaggagggtctagatcctcttgacagccttacttcactctggactgtgccatctcgaggaggcagtgacagcaatggcagttactgtcaacaggtggacattgaaaagctgaaaatcaacggagactctgaagcactgagtcctcacggtgagtccacggatacagcctctgactttgaaggtcacctcacggaggacagcagtgaggctgacactagagaagctgcagtgacaaagggatcttcggtggacaaggatgagaaacccaattggaaccaatctgccccactgtccaaggtgaatggtgacatgcgtctggttacaaggacagatgggatggttgctcctcagagctgggtgtctcgagtatgtgcggtccgccaaaagatcccagattccctactgctggccagtactgagtaccagccaagagccgtgtgcctgtccatgcctgggtcctcagtggaggccactaacccacttgtgatgcagttgctgcagggtagcttgcccctagagaaggttcttccaccagcccacgatgacagcatgtcagaatccccacaagtaccacttacaaaagaccagagccatggctcgctacgcatgggatctttacatggtcttggaaaaaacagtggcatggttgatggaagcagccccagttctttaagggctttgaaggagcctcttctgccagatagctgtgaaacaggcactggtcttgccaggattgaggccacccaggctcctggagcaccccaaaagaattgcaaggcagtcccaagttttgactccctccatccagtgacaaatcccattacatcctctaggaaactggaagaaatggattccaaagagcagttctcttcctttagttgtgaagatcagaaggaagtccgtgctatgtcacaggacagtaattcaaatgctgctccaggaaagagcccaggagatcttactacctcgagaacacctcgtttctcatctccaaatgtgatctcctttggtccagagcagacaggtcgggccctgggtgatcagagcaatgttacaggccaagggaagaagctttttggctctgggaatgtggctgcaacccttcagcgccccaggcctgcggacccgatgcctcttcctgctgagatccctccagtttttcccagtgggaagttgggaccaagcacaaactccatgtctggtggggtacagactccaagggaagactgggctccaaagccacatgcctttgttggcagcgtcaagaatgagaagacttttgtggggggtcctcttaaggcaaatgccgagaacaggaaagctactgggcatagtcccctggaactggtgggtcacttggaagggatgccctttgtcatggacttgcccttctggaaattaccccgagagccagggaaggggctcagtgagcctctggagccttcttctctcccctcccaactcagcatcaagcaggcattttatgggaagctttctaaactccaactgagttccaccagctttaattattcctctagctctcccacctttcccaaaggccttgctggaagtgtggtgcagctgagccacaaagcaaactttggtgcgagccacagtgcatcactttccttgcaaatgttcactgacagcagcacggtggaaagcatctcgctccagtgtgcgtgcagcctgaaagccatgatcatgtgccaaggctgcggtgcgttctgtcacgatgactgtattggaccctcaaagctctgtgtattgtgccttgtggtgagataataaattatggccatgggaaacattgtatatttagtgtgtgtattttgataatgattgatcttaaatctgtatacagaatatcattgatataatactctttaggcaggagcactcttgccttcccccaaaatttacactgctaaagccctctgtcacttggcgacccttctggtcttgctggaggggtttcctgggtataacccattgggctgcccaaggccagccagcctgagctctcctgcaagacagagcctgatgtggcacggagtggggttgcggggggtggggggactgcctgactcccagagggacttgaaactgaagcaagaaggttgcattctccaccaagggagttaacctacctgaactaagtagaaatgccagtcttccactaccccctccctgccatcttttcttctgctactttggggagttgatggccaggaaagaagccagcacagggttaaagtaactcctggcattgcccaccagggggctggtgcacctgctgacctcagggtcacagttgagtcatttgccagttgacggagcaagtttgaccttggttctgttgctgaagcaaatttggaacttttctgtctcagtgtgatccactaacccacaggatcatttggaaccttgaatagctctgcttggacaatggggttggggaatagggttgtctttcctatgaaaatgccatctgtagaccttgtgagtcagccgtccagatgtttgcaggtgaattcctctgcttgacatcctccctgtcactttggaccctatgggagtgggcatctccacgcacctgtgtatgtgaaagtcattttacatttcaaagcagtgtgtgtttcttatttttatatttttaactctttattcttggatgtataaagtgaactttttggcttctgtaagtatgctctatgcacctctaatgttttatcatgtatttatatgttgtacacagtactggctgattctgtaaatggatgtattgtacagagaacatgaacgtctcttcctaattttacatcttcagcatcattgcattaaagtggtgtaatctccttctctacatctgttgtcagagccactgagtgctgtgctgctcgacgtgagggtgaaatgattgacttgtgacctgccaggttgcccgatgccctgttgggtcaccggctggacctgctgcagcctgcagagccacagtcagcctgcccacatgccaccgagcaaacgcatcttgcttttcacatctctcctcctacagccttaatggctgcttgctgccatatgtgacaaatcaccaccaccagtgttaagtgcttctggattcatgggtgagttccctgggcagcccccaggaaggccttccagatctggctccagggtcaccacctgtcacagcaatacctgggaccatgctctcctgggactgtgaggctccttttgacgtacttttgacatcaggcaggtttgggaagaaacaaagccatgcctgctcctgcctctctcccaacatgtttccagcaagtagatgcccctgtgtgtgttttcccttgccttgtttcctgccttatatcttgtatttcgacttattacagagttgagggttcttgcttaatttagatcaagtataaaatttgtatgacttcaagtctcattttatctgaaaggtttttttctcatttaatctgatgtggcattttcgtcatctgaagcatgagtgacaagttgggaatgatgtggtgatttagaatgcagtattggccaagtccaagttgtcaacttaagcgtctgtttaccaaagaccgggaacaggggcccaaacatgtccagtcctcttcttccctctgctggaacctttggggacactcaagggtacagtttgacactgatctggtccatgaggctgcccagagaaagcactgcttctgtatgtctcttgtggtattggaacaataaacccgtacaacctgca

SEQ ID NO: 27 - DNA, 114717 bp, Homo sapiens
  source 1..114717: mol_type = genomic DNA
gcagtgggctctggcggaggtcgggagaactgcagggcgaaggccgccgggggctccgcgggctgcggggggaggcacttgacaccggcccggggagaggaggggccgctgtccctgcggccagtgctggatgcggggacccagcgcagaagcagcgccaggtagcggggcgcccagcgtggggagggctgcggcgaggcccggagctcggctacgactcgaggggctctgagtggccgggagcgcggccgaggcggggcgcccggggcccggcccccggtctgggcggtgcggggtccagacctccgccaagcggcgtgcggggaccccggcggggccgcagaggccgggaagtcgcgccgagggcgctaggaccggccgcgcgcccaggccgctcccgccgcggcgccccggggccccccagtcggcgctgcctcccgggccggctccgcagcgccggggccggggccagcgcggccgcggggggcgagcggcgggcggaggcctgggcccgcgggcggccggagggagggaaggaggaaggcggagagaaggggtgggagggggggacccggcggaggaggcggagagagggagcgcgacagcgagcggagggagggagcgagcgagcgaggcaggcaggcgggccgggagggagggagggcgcgcgggcggcggcggcggcgagagcagaggacgagccgggacgcggcgccgcggcaccagggcgcgcagccgggccggcccgaccccaccggccatacggtaatgagcgccgctgctggcggcccggaaggccggcccggaggcggcggggagccggggagccgggcggggggcgccgcggctggcagtgcccggctcggggcacgctcccggacccctgcaactcggcgccgggcgcccgtggggccgggactgcaggcggggacggcgggccgggctggccggggcggcgcgggggatttgagggtttgtttatgtccccgcgggctcggcaggcgccccctcccgtcccgcccgctcccggcgcggacccccctccccgcccccgcccggctccgggctcaagttcaagtctatacaatgccgcggggggcccctccaccgccggcctgcgccgcggtgcccggctcgccgctgccgcaggggccccggccgggccgcccccgccttccgggctctgccccagccccgcggccccgcctggagcccgcggccagggcgccggagggcgcggaccccgacccagaaggatcggcccccgcggcggcccgggggagcgggggagggggcgggggccgggccccgcgcaggctccgcggcggcggcagaaaatggcagcctggcacgacccggccccccgcccctccccccggcgcccggccctcctcggccccgcggggcgggggacccgggaggcccccgggggcgggccgaggggtgatggcggggccgggccggggagcgccgcgccgcgatcctcctccggccacctgtggctgggggtgcggggcgccggcggggccgggaggggcggcggggccggggagagcgcccgccttgggggcacaccttcccccggagtgccggcggcacggagaggggtggtcgtgcccggctcctccctgtccccaccgcgggccgtgggtgcccgggcggccgcggtggcggcgccgcaatgtgggggcggggcgggggcgggcgaggatcccgggcccgggcccgggccgcgggggccggaaaacgcgggggtggaagtagcggggccgggaccgggagcggcgcagggggggtacgcccggggtcgcccctccagcacccccgcgcacactcccacgcacgcggacacactcacgcacacgcaccccgccgggccggtgcagacgtgtccttgaaagcttcgcatctgcacagtgaaggacgaggcctcgccgcggctgcggagcggggtcttcggccccacgtccccgcaccccgggagacctcgagcttcggagccgcaggcggcggctgtccgtgtcccgccctcaccccagttcccctcccccaggccgccctgccttccctccccttcggcgcggaccgggctggcgcgggtccagggcccgcccctggcctcccccagcccctcatcgctggggcccgcggccctggggctgccaaactgcttttcgctttcctgaagcctcccttcttgggggcaccctctcccaccccagcgcccctctgccatttctccccctctcctcatcccctccgtcctggcaccctctctccgctgcacggccagttgaggaggtgttggcgccctgggcggggagcagtgccctctggttcccaggggctgcctcccccggctcctgaggggagccccctgcggctgcaccttgacccaacatggcgtctggcacccagcagggcagctctggcctgcggcaggcgggagggtgggcagggccgagccccagggccgcgcagctaccccccctccaaaccagcggggacagaagccttggggagaagcgctgtccgccctgtgacccaacttgcccctcactgccgtgcccagagactgccatcagcatccctgagggcctgctcagtcttcatgtttcaccctaggctgccagcggctgaacccgcctcctgggcgggcggctacgaagggatcctgggatcaggtgctgctccagtgtcgcctcctccctcgcgggcagatgtgacagctgcagccaggggagctattcgccttgctattgcctgggtgttaggactggcctgtgggatttggggggccaggcatcccggggaccctctttggttggctagcaggggccatctccaggtagttttctgagctgcctgcctttggagagttgggttgtagggagttcctgctgggtttctggggcaaaatggtccccctctgcctccaggttctaccgaggccctgcttgggctgtgacaggtggctctgtcaaggtgggagtctgtgtgtgcacatgtgctggctccaggcgagggaccaggctggcctgtgcagcagctctggcacctgcctgcctccctggtgggtccccctcctctgtgcagctcctccccagataagcagctgcggattcaggccgaccaaacttccctcgttaccccacccaggcaccagtgccttccataggagactgaccctaagaaaggtgaattccttctagactcaaggagtggactggtccacagggctgccctctgagtccttcctggagaagcctaggaaaggacgccagcagccatctgtcagggtgtctgcggacaggattcctctgtggggtgggggttggactagagccggaaggtccattccagctttgacattctatggctctgattctagtggtggaatttcaggcctagcaatggggacagggtggtctgtttttcggcacatggcagtaataggggaaggcttccaaggagaaggagatatgagagagatcagggcgcccagcgagttgggccttgggaaggagaggcagtgtgaggagggtccctgcaagagacgggttggtcacgtccgactctgcagagggtgtaaaaagtacagcctcctttaagcctcggcacgtgtgtatcctgtaggtttgtgtataaatccctgtgtgtggataatttctgaggtttccttttcaagtccaattcacagtaagctcctcagtcccttatggatgttttactatcgctttcacacccacctctgccagtggttctcaaaaagctaggaagacaggacaaacggtaaaatccttgtccagggtcaccggcaggaagaagtggagccaggcctgcactcgggggaagatgccccatgctatacctcttacctgggctgctgcatgtgtctgtccccgctctcagtttgtgatctccaggaaacaggggccttgcctccctgggtccccattggacaagcggtgaggcctccgagggtgtctgctgagcgaggggagcgcactgccctccagtcctctccccgaggcagcgaggcaggttagtgctgggctcccttccccctgctgctgtgcgccgctgcccagcggccactgcctacttacaggccagctcctcagggtcacacctccctgaaagacccaagttctttcccagcaagagccagactcccccaaggcttctctttttgggatccaggtagaaagaggactctaatgggtgagttaaaacagaccaccctctccccatctttgggcccgaaatgtcagcattagaacccaaaccttagaattgcagtctgaaagaggagtttaagctctgagagctcttagtgctgggtacgggactgtttgtgggaggtggtgcccagttcttcccagggttgtccatgctacccagagcctgaggcctggagggccattcacgtgccagtcacatgcctctcccagggtacttcctgctgcctccaacttgctggcctggggtcctggacttggtgctcgtatcctccccaccccgtgccccactgggagttgagtgtgtctctggcctccagcaggtagggccagagcatctaccccaatgccattccccatgctgcggccctcggggaatagcttctacttttgtttgaatattagtatgtgagcagagagcccgtcagaagcccacctggcctgaggctgggtttctgcagggactttgctgttcctgagcagggctttaatggtctactaaaggtgccagatccctttccactttaattttgagggctcattttcaatcatttctctctgacgatttttccctaagaacatgaccaaatctgagatccatgaagtgtcagcctgagatttatgtctgtatccgagcctgcctccacttggctaagtgtgtgggcaagacgaggttcagtgcacatgtgtctgtgcatgatgtgtgtgtacgtggtgtgccgccacctgtggatgggtaggacttgcttccacatgagccagtgtcaaggcagcttgaggccctcgagtcacagggagctgcactctgacacctcccagcggcaggaacttgggcaacaactcgattccctgggcctcagtttcctcacctgtaacatgggagtgtgcgtggattctgcttacaggtggttgtgaggatggagtgagatcatgtgcgttacagccttagaggagcgggagccctcctgttagctgtgtctgttttcatcatttgggtggctcacgtggactagcactgcaggaagtgctctaagaagcctcgtccaccttcctgtgtcttttgcagaatcttggtcagatgaggaaagtgccgattctgattaaaaatcctgggaggtgggaggcttctcagcgacacttagcaatctgttgagttgaacaaatctcgtgtcaacaggttgttttgcttccacctgaaatccctcccgctgtagtttcagctcgttattctcatcctgtcttgagtgcagcagcactttcccaccgtattcgcttccattttccagagatgtgctgagcatttcctgtaagccatgccctgtgctggctttgaggggacacggtgtgtttaggatgcgcccagctaacgaagaactgcagagtgggactggggtcactggagagggagaaataaagtgctcaaaggagagtgaggtcacaaccaactaaggtcatcagggaaggcttcttgaaggaggtggcatctcagaagcgccccagagcacagaagtggggcttcaggggcaggacatcattgtgggtggctgtagtgcccgggaggtggactggggcctgggttgtgctggaggccaggctgaggccctgccttggtttggggaggagatccctgcactccggaactcctctgtggcccacggaggatcgctctgaactgcctcagcgtggcggccagtgggggtaggggtggagagaggacagcagggagggaggggaaggggccaaatgagctcatttggtgtcggatcctgattctttctattccatagaccagagcttaagagctcagacttggagagcaacagaacagggtttaaattacactgaccccagggggttgtgacgattaagggggaggccagggtcaggtctggtctgcgagcgagtgatagcagtcatggttgaggatgtggcaggtggagaagggtcccttttctggcctcccagaggtctccatgtccccccttgcccactccatcaggctgaggcgccccagcatccagaacattcctgcaaggggctgccagtgccagggaaatgggggtgaagacgtcaggttcctgtctctgcagcctgaccctgtcccccaacacacacgctgggcagaaccgagccaccaacccagaggggaggtgccctgcagacggcccaggcccccaggcatgagtgcaggtgcctggcagccccctgcaagaacagctggtgggcagggccttctgggccaaatctcccttgctgatcacatcacaggccaccaacctgtctggaggacagcaagggtgcttggtgggcagtggaggtgggggtaagggaggtgcagacgcctggggctttcctagctaaatcccagtatggagtggtttgtttttttttttcagcagcagctctatgttgcctactcctgtcctactgtggtcctctctgacccttaaaggctgcccagccttatttagatatatgaccaggttcctattttctctttcctgtggtcacggccctgctcaccaccatccctattgatctttcctctctgcttctgtttcaaatgtcaaggtcatttttaattctgagccagttattagctgccccgtctgagtgcacagaggggacggaggaccatctcttatagccagtaaccggagagctggaacttgggtctcctgactcctagtctaatgctcttcttactataatgaaaggggaggggggctcagtgcagatgttcgagatctgatagatcagctgggtcatgggttccagtgatccttccttgaaaaggtaaaaagatgcttagagattatggggtgtgggttagatttgcatctcactaagctgttgagtgtgggctgtgtcctggggaatacttgggaacggccatggtctgggggcaggaggccttcggagtgtccctcccccgccactccctggaggaccccgggagacacaggcgagttcgcccctgcccctgccaagccagtgggattccccagcaccccagtgagcagaacctgtttggaaagaggcttctggaaggcctgcacgattacttgtataggttcccgaagtgataaataaatgcatgggtaaaggggaatccacttagattccctaaaagcctttgaaacaaggtggtaggcgagtctgttctgatgtgttagatccccatgcagtagtgttgtggttaaaaaaaaaaagttcatagtgtaatctcagttttgtgaaattctttaaaatcgatacaaatatacacatgtttaggtttgcacatgcatggaaaagttgttctggaaagatacacacaaacctgtcaacaagtttttgctttttaactttgtacactttcgttttctttgaaaaaatgtaaatgaacttgagttactttcttaatgacaacaacaaaaataaaactacactttttaaaagcctgtggatgagaaagtagaaaaaagaattaagtcacctaaggctgagggatggactcttttttataatggataagaaactggtttagagactgacagcaaggggtaagcttactcaggtacgtgtcttttccggaaaggttgcagggagaaatatcgcagatattaaggcctctgagcaaaggcctaggctggtagggattaggtgagagcagaaaataggccgctgactgcatgcaccctggcccacgccagggacaggggacaaagttagctttataggatggcgacttgaaactcatttctgacccaaagtggagaacaaatcactgaggatcttcccccaaaagcatcagtctgatgggctgttgtggcccaaatggttcctggggaactggaggtataactggggcccggcccgggtatggagctcaggtgacccagccctggcatagtgagagcctcttgggaggccacacccaagggagacaaagtggggcttcaggaggccccacgaaggggtagaaggggtgtctaggaacttgcttggcacgtgggttcaactctgatctaggggtccctgagcaggtggtgatcacccagatgatggggtggagaggtgaggtgggggcaaatacaagttcctctgtctgcagtcgtcttgctaccccaggaagctcagggctggttgcttagcaacagcacttaccaccctcttccaccttaccctcccgcaccccacccatgggtgcctcttggggcttcccttcccccgaggctgcccctgttcaataggacatgggaagggtctgtgcacttttttgaccctgcccctggaataaggcaggaacagacataaaaggacctatgacttggaggcaagcacagaactggcccctctacaaaccagctgtttaatccaaggctggtaggggacagatgtgcagggcaaaaaaggcgttcgggagtcaggccgacctggaggactgtctcagccccatttgtgctcagttgctgctggccccaaaccacctggagctgcaggtttttcatctgtaaatcggggataagaatagcagtctcatcttacaggggttgcctgaatgacgtgcaatattttgtgaaacagctagcactatgcctggcacagagaaggtgctcaggaggcatgagctgcttttctttacgtgggggttgttccagggctgcctggttagggtgacccccacaaagccctgctgctctgtaaggccctgctgctctgtaagaccctgcaagtggccaggtgcagtggctcacgcctgtaatcccagcgctttgggaggccgaggcgggtggatcatctgaggtgaggagttcgagaccagcctggccaacatggtgaaaccacctctctactaaaaatacaaaaatttagctgggtgtgatggtgcacgcctgtaatcccagctactccggaggctgaggcaggagaatcacttgaacccgggaggtggaggttgcagtgagccgagatcgcgccactgcactccagcctgggcgacagagtgagacttcgtctcaaaagaaaaaaaaaaaaaagacctgcaagtgtggcctgtcctccccatggcccctcacggtgaggggaagagataatcaagttgagaatgacccccaagcccatggtggactctgggctgggagtcaggggccacaggggagtggcagggatagagaaagatgtctctgtcctccttagcttccgttatctgacccaggtccctttttccagctgccaataagttgtccctttttctcttagcctttctgcacctccacttcttcctgtgaagccaggatttgcctgtaccttttttgatctcttcagagtcccctgtccatgtggggacccagtagcctgctcaaggcctgggccctctttccctccctgcccactggtctctttggagctaagcggagagtggcctttgctgggttctggctctgccccacggcctggaggcaggggagggtctagaccacagcccggcctggcagctggcacgcagcaagggctcctaatggttttattaccagcaacaatgtggtgcttgttgccatccacgcacaggctggtggttcccagcccaactggtggtggcctgagggccctgcctggtactggaagctcagctgggaagcccagtggggctgggcagtcagtcacctctgcgttttcttctgtcaacaccctgccagcagcctggaatcagagggagccagggcacggcatggcgagatgctggcccccgagttggcaggcctggcagcacttgagttttctttccagaggtccctgcctcagatccacaaatgatgttcatccattcatctaagagacctactgtgtgccagacacaggctgcatagatgaagggggtcacactcatctcaatgaccctgcagcttaggtggggtctggcagggcggaaggacccctggcacaccatgtccagggattaccagggctcagagaaggaagtcggattcagggggagagaagatctggaagggctttctaaaggaggtgacgcttgagctgagtctcgaagcggagggagcagttagctaggtggacgacggcactttctggccccatgtggctgcagtgtagggaacgatgggatacgaagccaggggcctgggccgggggacatggaaggcgggatgttatgaacggacgttgggcttgggatttatatttgagaaggatcattctagggcatcgaggacgagagctcgggcaatagggtttaggggcagggagcccgggtggaaggctggcacagaagtccggggcagcaatgtgggcctggacagggtgtgcgtggtgtgacatttaggaagtgcagtcagtaggaacaggcgggataaaagcaatgaggggagttggggtggctgtctggtttctagctcagccaactgtgtgggtggggatgcccttcatggagggggcatgggttggggggagtcggagagagcaggaggtgcctcccacatggagctgggttgtagctggggcttgagggcgggtgggatttccccagggggaggctgagccaggccaggcttgcagaaggaggctggagcctgagtcataaggcctggaggcggggtgccttgtgcgatggatgagaaaggcaggtgcatttcagtctgtaactggtacgacccctgcagatgtttgaaggaagattttgaggaaggtgctcgcacccttgcattgggaatgctgatgagttagcgcctctacttcacagtgatttccccaaaagatgagctgcaaggcctagaccctggccctccacccaggacctgcggcgagcatcatcaccgtctgagaatctttgggtgggctggaagttgatgtcaagctgtccagcctttccgtcactctccattcagaggcaggtctgcggagagagataggagggacaagctggggacagcatttttggaaagcaaacagaaaagaagcaaaaactgttcctctaaaatagaaaggaggcttatgtgtgttatgtgaaggataatgtggaaggctatttccaaagaatttctcaaaaaatgacgtgagccatgtcagcatcattgaatttagatagaggccaggagcagtgtggcatcgtgctggggccaccaggctggggactggacagatgatggtttcacctcacccctgccacctccttgctgcgtgtcattggaagatcactcaacctttcttaggctcagttttaccatctgtaaaatggacataacttcacaggctggtgttgtttgttttgagcattgcttcttggactaaacctggatattttttccttccctccctcgcttctgcacttcctctggttctttgcctcccaagaagcctgtttcctcctgttccctccaaaactccaagttttgagttcccccttcctgggaaggtccattcgtcatgttctattgactctcttgttcccaattattccctgcaaacctggttcacccagtgtatttgaagagcatctactgagctaaatgggagttattgggggtcagttcctgttttctttttaataaggggagggagggtctgtgcagagctgtccgggagagcagagggcatgggggacaggggggatgttgcaggacccagtgggaccacagctgggaccttgccctgtagctggctcagtgtgccaggggataggggcacagagccctggtgggtctgggagatgagcttgggaaggacttggcctggacatgggctaggctgccctgctttccgagccagacctcaggagatggcagagagggccgcttcctctgtgagggccgtacatgatggcccttgggctgtatggtggccttgtgacatgcagccacacccctggggagacctttgaagcatctctgcattctggaggatctgctgggaagatggcttcgcagcggggctcttgttgatgctgccaagaaggccgggtgggagtcgggctgtctctggctgggggtggggtgggggctgcggtccatccgagtgtggaaatccacgtttctctggtttgtgcagacctgtgcaagcagccatgctgcaggactggccccctggaagtggggcatacatgaactcagggtgtggcgttgtaatcaaatgaaatgtacagcataattaatctggcatacccttaatgatatgcataattgtgctgcatggtaattaggtgtaaatggtaagcagttcacataaatgtttttgtaattggtattaattttgcagaggcaaaccaagtttatgtggtttttctcttcttgctctgttagttttctagtacaggcatttccaagcttttggaggaattctattgcaaatgtttgtttatagggcaatgttttgtgttttctctcagaacaatgttatgaatggttatgaggttcccaagctggtttccaaatgcctctttggcccatacagtaggtgaaatgtgcaacgcgtgtgcctgtgtcggaaggcccgcccagcatcctctggctccaggtgccccagcgtgtgcagcatgaggggggctgtgtgcgtgtgtgaacctgtgcgcctgggtcatctctgcatttgtctaggtatatctctgtgtgtgcatctatgtgtgtttgtatctgtgtgtctgtgtgtgtgtgtgtgtgtgtgtgtgtgtgtgtgtgtgtggggtctgcatgactgtgagagactcctgaggccacagtggcaggtagacattctctggtaaatacattccccattctagtggcacggggcctgggctggagacttaggggcatcagctgggagggggagcctggcacagcagccgttcctaggagtgagcacaagccactctgaagtgtggtctgaagccagagtctaggtttcctaagaacatatggcttgcctttctttctgatcaagcacataataatttttaaatgatcatttattgagtacctgccacgtgccaggccctatgttaaacacttgactcagattccctcctatcttaaaatagacttatgaaatagggactctttctcaccccgctttacaacttgggaaactgaggcccgaatctctcagtgacagagccagggtttgaggcagggactgctggaggcagggcccatggttctgagatgtgctgctcagaaccctgggctcttctccctccctcggccccctccacaaatggtttgtttttgttgggacagacgatgaggagaaaggcggccctgtgggaaatctgagagcagcgagctcccttcctgctgaattggttggggtggtgggggacgagctccaacagccgctcccaagccctgaagaccctttgggggacctcctggttttgacccaagagggagctggtttaggaagtggggttgggggactgtggccatggacggtgtggggagagggtggtcacaggccagacaaggactcagcccccctgattcctgtggttcactgatagcctaggccctcaccaaccccttcaccttggggatctggggcaggtgagcctgcagacccacagcaggtgtatgttctgccctcggagctcctgcaaacagcctttccactgacgcagtgccttgggggctctgccaagcgacccctagaatggggattgtggggggtcgctctaggcaccgcagcactgtgctggggatgttgcagctgcctgggagtgacttcacacagtcctctctgcctccagggtcacccggaacggggttgcgggggacaagctgtgggcgggggggacgggcagcaagatacaacgcatgaactctgacctcgtctctttgagaaatcagacacatctatatacgcatggaacactcaatgacccctgggtgggtggtcgaggagaaggggacggtgttttgggggaggagagatgttgggttgaaggactgggagagaggagctgagtgggccctgtgggagagggtgggagccaggggtgtcattccgggacagcagccgggcctggaatgatgcagctgggagaggtgggggcacggccagagcagtgggcggaggctgcgttgtagccagccctggtgagaccaggccagagcgtatattttaccacgtgtgcaaatctttggcatcaccggatttttaaaaataatagctttaagatataattcacattccatacagttcacccatataagtgtacagttcgatggttttagtacattcacaatcaactttttaaaattgtcatgaggtgtcataggttttcatagagggagccatgtgtgggcaatccttgctgatagggtggggtgggaggtagagatgccagaggcaggtggacccgctccagggcatctaccaggttattgtgccacagacacaagaggtacaaagggcctggagggaatagggggacgagagaagcccaaggaccaggtacctgctggatgtgaggacccaggggaaaggtgggacctgagttttctccagggtttgggacttaggcagctgaagggaggggaatgttacttccacatagagggaaaaaccagaaaatagagccggctctgaaagaagcatgttctggaagagtaggattttagggactgcaagtcatccagttgggatgaaaataaatagagctggagcccctggcagtaggaggcgtatgttaacttatgcccaggtgtcttagaggaagagcatttagagagaagctgaaggtagaacatgacctttgaggagcacccaccgtgcagaagtgggatcaagactcccggacagccccagagacaaagctgcagaatctgaggctggagacccagaagcaaggagccagatggagtcatggctgggagagaacacacagaaggcagttgctggtgagctccagaagcaggggtctcctttcactccagggcccggctccacaatggcactcaagaaatggctgttgagtgagtgcttgaagcagggagtgattagctggccaggatgttgaattgagctcaagggccatgaggatggaaaaaagccctttggggccgcgggtgactgtatcagtcagggtccattcagaagacagaaaccacatggtgattggaacaaggaaagtttcacataacgaattattaactctaattggattggaataatgaggaattggctagtaagaagtaaaaataactccaaagaatacaaaaataatagatatgagaacagcccctacctctagggttgccacagagtgcccaaggaagaggccccgcagggttgagatccacaccttgatggagagggtgtggcgatggctccctggatggcaacgtctctgtaatgccatgttggcagaacttgctggaaatccaccctctggaatttactggaaatccatctttttgggtcccaaggaaagttgtttgtgggaggtatcttgctggaagcacaccgctgctacgttttcccaggggagtgctgggagaagctcctggctgttgggtactcctgactgacgtgttgctggagccaggtgctggagaagctgtctgcattgcaacagcctggcactcctgcaggagctgggcactggaaagctgcatgctggagaagctgcagcagcacaggagctgggtgctgggaagcttcatatctggcaagaccctgatgctgggaagcgcccatgcagcaagatctggctgctagagaagttgtatacaatgcaggaacccaacaagagcacactgggtctagaaaggaaacccacctcctccttcaatgcctcttcagtgccctccacagttgtgccagctgacaaaggaaaagtagttaaagggcccagctccatcttcacagcacaggcaatgatgggcgaataaactgataactggcacagtttacccctttggctgcacagcttccatttgcacctttctacaacatctgaactcccatacaaaaacaaacaactggatgtctacctatcaagatagttatccttcttatgaaattctcacccttttgcccagagagccccaaccatcattctaccaatccctggctacaataacttctcaaagtcagtcacagtctcactgaatattctgttacctaaagactacattataaagctaactgtcaacaacttgtatagaaaagaatgggaaggagaaaaaaagaagaaaatggttaccatattgcaaacacaccacacacacacacacacacacacacacacacacacacacacacatatatatcaagttaatagaaaatgtgcaaaaccactagaggcctggtttctgcatttgttcacaaggctacactttacacttgctacctggcttcattgtcttcttatcccattctgtgtttcctccacactcagccagagccttagcaggtcatgcttcttttacttggtggagtaacccaaaccctcattcccaaaggacctgaatccttagttatcccgcttttgttgttgttaccataactttccattcagccttcctttgggcatggacgttcttaagaggtgcctgagagaattcgctgggttccagacacagtcttgcttggctctgtggctgatcagcaatccagttttctccttggtaactaggatcaatcactccagacagtggcataactccttttttccactgtcagttcagtggcgtaaggagttcgagaaagccaagtgggtgtgtgtgtcttctctcccagggaaagcattataatatgtccctggtgggagcactctcccccgaggacgaggtcagcatccccagaaggttttagagttgtggggtggggaaacagggccacgcgcaggggactaagtgatagaggctctggagctggcagctgtggaccatgctgtagagaaggttggaaaagtagagaaacatgcagatgaccgtgggccgcttccgctgtggatgataaaatcagacccatgctggtcactgccagaccccatccaggaccatgtgtctgattctcacatggactctgtgccaccctgtgcccctgactcccaggccgtgggaagcccatctgacccacagcacgtgtgacccctagacagggcttctcctggcttgcagcacttggaaatcggtgctgccttacggaagtctgtgttggctcattttggctcctgagaacaacttcagctccttaacagcatctattttaggtataaatcccccccccgctcacttttggggctgtgtcatggcaaccattagtgtgagcaaagcagtctgactcagactcccatgtggccgccgtagtggcagcaggtttggtgtgatcataagtgctcctgctggtggtgccagtgctggtggtgccagtgctggtggtgccatggcctcgagagtggtgtgagagccacaagccgtcttattgtgactctagagccagataaatcagctttgcagagggcaggactgctggcctgtcaccagtggcttttggtattctatggacagctggtagtttgggtgcaagtaatgtttctgaaagtcagggcaatggaatggtgatggccatctcactggaaaatggcaccaagacccccctccctctccctcctccttatccatccccagtcttatgaccaagagaggttgtgacaggagggggaagagttgacagatagacatctccgtctaggaggtctcctccactttctcctggcccctagctgacccctaccctagcagatgattgggcagggggtgggggtggggcacaagatactctgctgccttctgtcacccacatttgcacctcctcctaagctcaaataggaaatttagccatgggactgttatctgtgggccagggtcaccccagaggcacaaacatcgatcatggcgctggctctgagatgtgtgtttagcatgctcattcatgaccaagaactgccacatttgcgaagggttctgagttacacggacctggatttataatcctgccctggggctttctagctgtgctaccctagagaggtattaaacctctttaagcctccatgtcttctataaaatggggataacaataggacctgcttcataggattgttttgaggattgaggatagtgcatgttccttagcacagtgcttggcacatggtaggtgctcaggaaatagctgcatgaatgaatgagtgaatgaccactgcattgggcagggcattgaatactgtgattagcatgctccagtgccctccttccccgctcccactgcagacaagcctgaccaggagagagagactgactggaaactttgttttctggccagtccgtgcccaaagttaatatagatctgcagcagtcacatgctgattgtgagagagggagtggagagctgtgagcttctgagtggggtggacctggactgtgcctggctccaccccttacccactgtgtgaccttgggcagttttattaatctctccatacctcattattctcatctggaaaatggggataaaaatagtacccacttcaaagggttggggtgaggtgtgaggattaatgagatgttgacattgtaaagctctgctcacggggcctggcatgtcggaggagcttaagtacaagctcctgcctgagcgaaggccacttagaggaactccagggatttcaagaaaggagagaaatctgcgggctggttgaatctgagagtaatcatcgttaagatttattgagtactttctctgtgccaggcgctcttggaagcatttttagggcattccattttaattttcacagcagcgctgtgaggtggaaagttctttctcccactcgacagctgagaaaactgaggcacagtttcgcctcaggccacacagctggtaagaaataaggccagtgttggaaaccccgcagttggcctcccagggagccctggaggggactgagattagcccttaggtcaggagggtgtcaaccacaagtcatagactcataccaaagtagtgaaagctcacagtccccggaagttcagagacgtccctgcctccaggaacagcaagaccaggggcccagacagtgactacaacgctttatccctctgtcttcatctctgagctcttcgttctcaggcaggctcacccacatggcgggaagacagcagctggcagcccctgactcacatcgaccatgttagcatccttccatcgcatcgataaaaaggttccagggaggcctctgattggctctgcctggtcacgtgctcaccacttgcctggggtgagggttgaggtcagcacaggcacaacgaaaaccttttgacaagcgtttcccaccgggaaggaggggaaggcatctaggtagagcagaacaaccagagtccactagaggaaaaggagaatgcaagcagagggagggagaaggacctgctacagaaatggtcagatatttttcagccattcagacagagaacttgagacggagaagaaggggcccttagggtgagaatattgggatcgtgtttggtggagtgaggatttaggtttgtgggccgtgacagttttggttttggaagaggataattatgtgcaagcccttgttgaggagagactgtggggtggcaggggggccagcagtcctcaatctgaagatggagaggaggcttgggtggtcatctcgggggtgctgatgaggctggggttggtgaggaagtgggggcggcagtggggacagtgtggtgcaggggagcagtcctgaggcttcaggagatggtggagatgtagctctttggtctgagaaggggctgagtgagcgggggttggggttacggatgcaggcagcactgacttgcgctgcccatgggctggacgcagcgggaatgatgtgtttggaggattaaacatttcgtgagggtggggggtggagagggaggtaccagtgttgtagctggtgatggaactctgtgtgggtgtgaggagctgatgatggttattctgtgggacgtgatgatttcatagaacaacagaggtggaaaggcctcagctacactcttatttatagagaaggaaatggaggccaaacgcaaccgatggctgcagttgggacgtaagccctttctcccatccccactctctctgtttaaaaattcatttttatttactcacagtagtacatgcatataatttaagaagtcgaatagtactaaaacctcctaacaaaatgcagtgattccttgcctactcctacccttatcctcttctcagaggcaaccgctttcgattctcttggctgtttcttccagcaattttcatctcatatttctgagtaaagtgcatatttttgattcatctatttagacattttccattgatttcctatttagtagatcctcttacatgtccaacctttctgctcttcagccatcctccttaaatattgacagtactcttcttatattttttcaaaaaattgtttgttgttgattgttttgagacaggatctcgatctttgcccaggctggagtgtggtggcacaatcacagctccctgcagcctcgacctcctgggctcgagcgatcctcccacatcagcctctcgagtagctgggactacaggcatgtgccaccacactcggctaattttgtaaatttttgtagagacaggatctccctgtgtcatcagactggtcccaagtgatcctcctgtcttggcctctcagagtgctgggattacaggcatgagccattgtgtccagcctcaaaaatgttttgtgcatttggtagccagaataatggcccccctaagatggccaggtcctaatcccctggacctgtgaatatgtcaccttacatggcaaaagggactttgcagatgtgattaaattaatgacctagagatgaggggattatcctggattatctgggtgggcccagtgtaatcagaagggtcattagaagtgaaagatggaagcagaagagtagtcagagcaggagatgttgatgaggcagagagtactgtgatgggaaaaagactggccatcgctggctttggagttggaaggggaccacgtggcaaggaatgagggcagcctctagatgttgaaaatgtcaaggaaatggattctccttagagtttccagaaaagaatgcagccctgccaacatcttgagtttagcctagtaaggagaatctttttttttttttactactgacttgcagaattgcaagataatttttttttttgagacggagtttcgcttttgttgcccagggtggagtacaatggtgccatctccgctcaccgcaacctccgcctcccaggttcaagtgattctcctgcctcagccttcccgagtagctgggattacaggaatgcaccatcatgcctggctaatttttgtatttttagtagagacggagtttctccatgttggtcaggctggtcttgaacttccgacctcaggtgatccaccctcctcggcctcccaaagtgctgggattacaggtgtgagccaccgcgcccggccgataatgtgttgttttaaggcattaattttgtggtacatacaaaagtaatttgtgtgtacatacatgcatatatacatatatatgtacacacacatatatttgttaaacctacacacagtgtttattttattatttcgataaaactattattcacagctgagccttgtggtgcactgtaatagtttcttttcgtttttttttccctgaagttaataattaccttgtttctcatctgccttatttttttgtacctgttgctaattcctaccacaacttctaatataattttatttttctttaacaatactgttaccttaggacttctttttctttttttctttttaactgacacataataattgtgcatctctatggggtacaatgtgatgtttcaatacatgcatacattgtgtaatgatcaaaacaggatagttggtatatccatcacctccagcatttatcacttctttgtggtgggcctaccacactttccaatagccatcagtattgctttccacacgggcagtcacagcaggtaaaagatgtttcagttccattttttaagttatctatcctctttcactctgggctagttgctttctaggcacattgtagctgatgctctgtgacttccttttgtggtcatctttggaattcccattactttccggttggaatctctgttttctggctttcgtgtcctcctcatgcttggctcactcccctcattagagcacatcttccaggacacacatctgtccatatcaaaatccattcatggctgggcacagtggctcacgcctctaatcccaggactttgggaggccgaggcgggtggatcacctgaggttaggagtttgagaccagcctggccaacatggcaaaatcccatctctactaaaaatgcaaaaattagccgggcatggcggcgggtgcctgtaatcccagctactcgtgaggctgaggcaagagaattgcttgaacccacaaggcagaggttgtggtgagccgagaatgcgccactgcactccagcctgggctagagagcaagggtccatctcaaaaaaaaaaaaaagaaagaaagaaaaaatctattcagttcctgggtttagatagcagagtatggccacgagcgatacctgtgtagttttgtcaggtaaaaagacagttcgacagcagagggcatggtgatgacggcactcgtgccccagtctcagtgggaaggctggccctctctggctcatgccaggtgactggcatgcggcctcttttccccttcatcctggagcctcccttcccatctctcctgtactggatcttatgcgtattagaggacatcttcctgtccttcctgggaaaggagcgtgaggcctcatatgtctgaagatgtctttactagggcccagcacggtggcttacgcctgtaatcccagcactttgggatgctgaggtgggaggatcacttgagcccaggagttcaagatcagcctgggcaacatagtgagaccccatctctatttaagaaaaaaaaaagaccccgggcgtggtggctcatgcctgtaatcccagcactttgggaggctaaggcgggcggatcaccaggtcaggagctcgagaccatcctggctaacatggtgaaaccccgtctctactagaaatacaaaaaattagccgggtgtggtggtgggcacctgtagtaccagctacttgggaggctgaggcaggagaatggcatgaacccgggaggtggaggttgcagtgagccgagatcccaccactgtactccagcctgggcgacagagcaagactccgtctcaaaaaaaaaaaaaaagaaagaaagaaaagaaaagggccaggcgtggtggctcacacctgtaatcctaacactttgggaggccgaggcaggcagatcacgaggtcaggagattgagaccatcctggctaacacggtgaaaccctgtctctactaaaaatagaaaaaaaattagtcaggcatggtggtgggcgcctgtagtcccggctacttgggaggctgaggctggagaatggcatgaacccctgaggcagagcttgcagtgagccgagattgcaccactgcactctagcctgggcgacagagcgagactccatctcaaaaaaaaaaaaaaaaagaaaagaaaaaagaaaatgtctttattttactctcacacttaaatgataatttggctgaaaataattctaattggaagattgtttaagatttggtttattctttaagaattttggctgggcacggtggctcacacctgtaattccagcactttgggaggccaaggtgggtggatcacttgaggtcaggagttcaagaccagcctggccaacatggcgaaagctgtctctactaaaaatacaagaattagccgggagtggtgacgggcgcctgtggtcccagctactcgggaggctgagaaatgagaatcacttgaacccaggaggcggaggttgcagtgagtggagatcatgccacttcactccagcctgggcaacagagaaagactccgtctcagaaaacaaaaacaaacaaaaaaacccaactgatcggaagctctgagaatgatagtggacttgttggttttcaagtctacataggctgacctgaataagtcattttgaccccattttggtactgatgttggtattggcatccttaaacctttcctcttagactcattcgattctccagagaaggaaatggttgtctggctgccagtattcttagagcccaatcggggaatagcctgagagactggacatcagcatttaaaataaccttcccggccggcgcggtggctcatgcctgtaatcccagcactttgggaggctgacgtgggcagatcacctgagattgggagttcgagaccagcctgaccaacatggagaaaccccatctctactaaaagtacaaaattagccaggcttggtggtacatgcctgtaatcccagctactcgggaggctgaggcaggagaatcgcttgaacccgcgaggcggaggttgcggtgagccgagatcgcggcattgcactccagcctgggcaacaggagtgaaactccatctcagaaaataaataaataaataaccttcctctccttggtttgatatatcatctttgtcttcagctgtgccaagtgactctgtgcaaagaccccctattttatgccctccagtgactacagataaaccttcaaacttgtagaagagggtctaggaattcactactctcactcagcctctcgtttctcacacaagcatcctcttcagccctagtgccagagtacctggtgctgtcatataagcatctgttgaagactgtgttatatatcagcgttcacacgcctgtctgaaatttggctgtcacaagtctgctttccagattccacaattttgttggtggtattccttctcttgttcagcctaatcttgtgattttatgtcttaaaaaatcttttggccaggtgtggtggctcatgcttggaatcccaacactttgggaggctgaggtgggaggatcctttgaagccatgtgttcaagaccagcctgggcaacatagggagaccctgtttctacaaaaaaaaaaaaaaaaagccaggtgtggtggtacatggctgtagtcccacctactcaggaggatgaggtgggaggatcacttgcacttgagcccaggagtttgaggctgcattgagctatgatactaccactgcatccagcctgggtgacagagcaagaacctgctctaaacaaacaaacaaaaaatgtttctattgcattgagtttagaaggtagtgagccttggtatgcatattcaaagctttcagttctttttccacaacagttttatagttgacgcagtggagatggtgatcatatcagctgtgatacgaagatgttttcatggtgggattccttgaaattttttaagtggctgttaacaaacattattcatggtcactgtggactctaaaaggaggaaataaaagctgcttttcatggtggtagggttcttggcaagggttttttggtggcagtggtggtggctgtgcagcccttggcccaggtgggaagaacgctttggtatcggaagcagccagggctgtgcggtggaaggaccgatgtggtggacacccgctgctgtgttgggactgtgggcccaggagagcccactccccatggctgggctgcctgaaggaagaatgaagcgtctacgggtaggtgggcaatgctatgtcaaggtctgggaccttggccaccaactggggccccctagggttgtgtagtacatggcccgcccagacacatactggaggattttaggtgatggacacgattttgagggactgctactgacagggctggtccattgagcaacacgaaagggttgacccaggtcagctgcattttctgtgaggacagtttctgcagctcagatgtcagagtcttgggtggaggaaggggaggaggaagagaatcttcctttcctttcctgtctggggtgtctgcccttggggccaatgccctgtgaagttctgcttttccctccagcttccttcctctccagagcttctctgaacctcgccacacccgtggcggcacagatgccctctgggtgtccctaagcctagggcagcatcaagacaaccctcccgccccgcttcctttgtgcctgggtctcccccgactccatttggggtgccctgtccttgtcagccccttgtgtgtccactgtgcctgtggggccgcaacagcttccttggccggtcagagctattaatagaaggggtcctgctctcccatttcattggtggggaaactgagggccggggaagcctccgtcaggagtagatcccacccctgcggactttgggctcaacgttctttccaatcagactctgcccctcttagcttctgaccctttcgaaggtttttgttttagtgtggctggagatgtctgtggtaacaaatggggagtgaggcttgacagggaatcactggctgctaagggctggcagccgagtgcttctgggttggaccagttctcttttgggatcttcagagttgcttctgtggtgtgtgttgggggacgtgaacccaacccctcagaactttagtgtcagagagatctgggttcatgcctctgatgggtcctggccttgttgatcttaccgtcacctgtaaactgggcacggcacagcagctgcccagatgcagagcacgagccaggccctgagtggcatgtggccgatggagctgctctccgctgccgtccaccctctcgggacccgggcagcaaaccttccttaagcccagcactgcgctgtgcctaggggtatggggaatggacgcagtgcacctgggctgcctcagggctcagctgtggggcaggtccgcttcaagcctttctgggttgacctgatggagctcttcctgtcctggccctaagaagctcctcctgccagctccttcctcccactaccccctggggcacagccagggctggggcacccactcccaggcaagaatgcacccctggagaaaggggctaggcctgtaggagagccaagcctgtgctttctcccggtcctggttttccagggagccctgactggagggggatgggaggagaagcccaggagggagggggagctggggaagcagaaccaggaactggaaggggaagggtgtgggtgggcccagatccagcctgcctcccgctctcccttgcctgccaatcccctcctgggccctggcacactcaaggcaggggcttggcggccgcctcctccgggggtggggagcaggggagctcttgtgtgtctctgagtgagggggctgagacagcaacacccccaccccagctgtggcctgagctgcaggctttgttatttctctggcttgaggcctacacctggagcagagggaagaaaagcccctaaactcagccgtgtgggttcagaggacccctcaagcccaccaactctgcaggcaaatgaggctggttcatggggtgggggagagagcagcctcactcctgcctcagtgtggccgtggcctgtggccttggccgtggccgtggcctctccctcctaggccagagccaaccaggcctgcagcctgagctcagacctcagctttctctgggcccaggcaccggtcctgaagtgaggtgacattcatttcactacatcctgtgactgcactcacttcctccacgtggcgggaggtgtgggggtgggggtgaggacagattagaaggaaaacgtagctgggctaggggcctctgggggaggctgggatttcccaggggctggttggggtgggagggaggatgcgggaagctttcaaaggagagagaggctgtgaatgaagagaagggaggaggcggaggcagctggaggtcccagaagtggcacccattggccgcagcgacacggaggggagactgctgccactccaaggctctcagaggctgctggggtaggaggtggccctgaagggagcctgcctcggtttctctgctctctggggtgctggtgaggccagaaggaggagcaacaccccaggcctcacaggccaggtgtggccctgggctgagggcctgacccagggagggtggggtgctccgccccactgatcctcttctctcccccacaggtggagccatcgaagcccccacccacaggctgacagaggcaccgttcaccagagggctcaacaccgggatctatgtttaagttttaactctcgcctccaaagaccacgataattccttccccaaagcccagcagccccccagccccgcgcagccccagcctgcctcccggcgcccagatgcccgccatgccctccagcggccccggggacaccagcagctctgctgcggagcgggaggaggaccgaaaggtgagcagcgggctctgctggggaccctctggcctgggagagggagagccctgtccctatgtgatgacagccggctcctcatactgagtgcatgaccgcatcactcagtgctcacgacagggtgtgggaccatcacatccctgtttgggtgatgaggaaacggaggcttgcagaggttcaattctggtgatggagcaggcatttggttccaggacccaggcttttaaccaccatggcattcgcacagtggctcaaagcccaggtctgtgccattgtgagggttcggcctcacagagagtcccagaacctggattccttctcggacccgcctcttactagccgtgggccttcacgtgaatcactttctctctcagctgcctctgtttccttaaggtgttcagtagcttctaggggaccaaagacaagttctggggtggggcacggcagacacgaggccgcgggggcccaccctcccacacagacggaggcacaggctgggctcagtgtggcgccagcacactttggagatggtgatttcctgtccccgccccccaccccagactccaggtgaatgtcgctgggttatctttggtatggcttttggccacggagctggtgacctccatgttttcagaatctagttgggggtggcctctccacccttctgctgaactccaggttgtgcctgaatcagtcagaggagattacatcagactgatgggagtccctgctgggtctccacaggctgtcttgggaccttaccaagctgtgtccccatgataggaaaagctgccccttaacccccagcactggactgtgttgccctctgtcagtggaatggtgaactcactgccaccctccaggggatcttatgatggccataggccttcctggcatgtctgtctgctgtgcctctgtccacagttccccctgcctgctccccttcccctgagtagcgctgggggttgtgctgcttggtggtgtctgggagtagctgggcatgctcatgggggcaccagttggttcttttggaacaaaggtggacacctggcaccagctttgggggccctgggcactctggggctagaggcagccagcaccctacctgaagggtgtcagctctgagacctccctcttgcgtgctcagctccttcctgggtttaccattcttcagagtgtgtttctgtttcctagttttccatcaatggttctatttggtccttggggacaactcttgaaacaaatggggcagggacagatggccccagtttacagatgaggaagctgagccccgctgggcagtggcaggtccagatgacagcctgttctcctgtctcccaggtccgagcccttcccacagccctacctgcataggtcctgctagggacacatgtacacatcccaggagtgcacatgacgcccagggacctgccttcccagcccagcaccctgtagagcaccgtgggggtgtgtgcacccagtcagccagagcaggtcagaaagccagcatgcgctgcggcccactgggcaggacgcccggggcgaccgatcaggctcctcccttctgaatccatgggaaaagaactgatagtcctggatacctgcgggcccctaggctgcccccgctcaggactcaagcagcatgaagacccaagccctttggccgtagagttggggacacctgccgctgtctgtgctctggagtggcaactgtggccatggctgactccctccccttcttgggcctggcttgctttctctgtcggataaggtgaccccgagtgtcgccccactctgggtccgatgtcctgacagttgctgacgcagctccccatcaatgcactggcagtcactggtcagctgctccccacaggtggggagggtggggacagctgggacaaactcctcctgttactggactgtactcccacgccgctgctgctgctctgggctgcggagccccaggccaccaccaatggacgggagcaggtcaggatctgcattataatagctcaggactgagaagcaacgctggaccaggagtcagaaatcccaggtcctagtcctgagtatgccacttactggagatgtgacttcaggccatctgggcttgtgtttgctcagctggaaaatggggatagtgactccagccttgcctgttttaaagggtggttgtggaagtcagataaggtcatgggcatggaagtgacctgcagacagcaaagtctggtccaattatgagggattacgattgctattattatctacccatagtaactgctctggctcaagcggcaacaaacacacacccagccctagatcccggtgctgatagagtgcacactcacaccccagcaggaggggtcatgccaaaagatggcagtgatctggatagataagggctgggccatagccaggcctcctccccagccgcccacacctacaccacgcatgctggctcacctggcacacttccccggcacctctgggctggctccagcacctccagggagccgcccagggagggtggggagagacactgacagccaccacagcagcccatgaaagccaggcctgcggcaggggtgctggtagctgcagaggggaagacacaggcttggtctgcagcccagggagcctggcacaggtgccaggggcactgtgcacaaaggaactgtttggcaagccagatggaaggggtgtggccaaccaatggcccttccactgcctggccccatgtggccctctagcctctgcccaccgccgccccccctccccgcccccgcccccagccccgcctccatgcacctttcactccagccccactcccatacttcctggagtctccaggtctttgtttatgctgctccctctgccgcatgggccctttccctaatctcttctcctccctggtcaaagcctcaaagcccaggctgtagttggccttgtattatagtttgcaggggaccagtggttctcaaagtgggatccccaaatgagcaacttcagcatcacctgggaaggcaaagctcaggccccattccagacctgctgagccagaactcgggggatgggacccagccgggcacctttaaccatccctccaggggatactgttgcgcacagatgttgagaactattggtgtagataacttctgcccactgctagcctggagcgcagggtcacggcccttttcttccatgtgaatcccccggacctggaatagcacagagtgggtgcttgggaaatattttacggatagaacggaaagaaaaatgagagtagccaagaccctcccacaggagaataatcccggagcccaggatcagggaggcggagtgcagagctgtgggtgctgggctgaggcagccatgctgtggctgggagtgtacagagtggcctgtgtgtgtccccctggcacctgccgcctgccctacccgccatccatatggtgggcagagcagggctgcttggggagatggggaggaggcaggccgagctctggggctgtggtcagggcgagtcagctggtaaatgttgccgcagcagagggagggggctggcctcatggagggtgggtggggaggcaagcagtggggaggctgagagcttagaggggagtatggagcagagggtggggggcagagggagcaggacaggggaccaagaacagtccaagggccacctcagatccccaaatatgaatcagaggggagtccacagccccaggccaggcccctgttctcagttctgtcccctttcctctggcctcagaaacacaatggctaagatgcattgaacgccgactgccgggtgctatgtgcatgcattatcttattccattctttttttgtttgagacggagtcttgctctgttgcccaggctggagtgcagtggcgtgatcttgtttcactgcaagctctgcctcccgggttcacgccattctcctgcctcagcctcctgagtagctgggactacaggcgcccgccatgcccagctaattttttgtatttttttttttagtagagacggggtttcaccatgttagccagcatggtctcgatctcctgacctcatgatccacccgcctcggcctctcaaagtgctgggattacaggcgtgagccaccacgccgggcctatcttatttaattcttacaattgccgcataaggtagacactattattttcatcattctatcttattaagggctaaactgaagctcagagaggttaagtcacttgcctagagtcacacagcttgttaagtgctagtgctgaggctggaatgaggctggcttctgcgctccttaggctgtctcctgcgtgaagggggatggagtgaacagtgccccccggtggtgatgtgatgagaattcgtagtcctcccctggacttttgctgcaggagaaagcctttcactgcataatggcgctcttgcccttggggagaagttgggggcacttcatctccagcaagaactgcacacaccacctgcgtgtacaggcacacacaccagatggaggcactgctgcagacatgcagcttcttaccatttcttcctgcctcctgccctccttccttccctgcctctccctccctggacatacagctcctcttttcttctcttcctccctccctttcttcagacataggtacctttccaggtagttagcatgtagcagggctccgcaaggctgttggatgaactgagtgaccgtgcttggagggcctagtagaggggtaggaagacatcgaggtgggtaagccatgcctgccccccaggggtgggtgaggcctgctgggcagctcctctgatcaagccttctcctgcctgtagtagaagctgcagctggggtgcatctttgtgctggggaaacacagcacttatgtgtttgcacatgtgcgtgcgtgtgtgtgtgtgtgtgtgtgtgtgtgtgtgtatgcatctgtgggcagcattggggctgcggttcctcctttcccctccccatcaaagtctggcccagaggcctggcagccactgatttctcgtgggctctctccactctggtctgcaagtacaagggcctccatccttgggtggggtccctgccagttccagtggcagtcattcttgctgcccgcggtctgtcacctgctcctgggcatgttctgcctttcttgcactcagatgcccccacatggtcacttagggctggctctttgcttgacagtggttgcggtaaggtcatttcaaagagcatccttcttagtatgtttaaatagctatagaaaagccgctgcaccctcacctcttcagagacccagccagcgataatggcgcgctcactctgatctcgggggagttaggagagggttgggcgtgcttagcaggtttgtgatgtaaacagtgcccatccctgagctgagcaggacctacattttgggcttcggtttctctaaaattctcttaaattggacattattacatgtcaaatgtagttaatcttggtcagtacagtcttggatgggggttgtttggcccctgcagaagggcctgtgttgaagctggactctgtcacgtgacttggaaacatggctgtgagcaggaaaacagctcctagttggcttgagaggttcagacgagatggtgtgtgcaagtggttaacaccaggacatagcatatgttaagttagctttataatttattttggatatctacctacccatccacccacccacctacctgcctacctacctacctacctacacctttgtggtttacacccactgccgccccatgaattgcaggggcgtgggagaagagtaggaaaccatgggtggggacagagtgtgggcttagagtccactgagagcctagcacgtgaagggaatgttgtctctctttgggtcatcagtcactaaaggcctgagttccctttatcccttgaacaaaagtaaaacaaggcaagtggtgatgagacctaactcctgatggcaggggctgtagatcccttcaggggcctaggtgaggggccacagtcacttgggtccctcctgctcttaatggccgggttagggatggagcccagccctgctgataaggtgagtcgcccagggccagggcaggaaaggccaggctcagctgtgcagaatagaagaatgggccaggcgcagtggctcacacctgaatcccagcactgtgggaggccaaggcagatggatcacttgaggtcaggagttggagaccagcctggccaacatggcaaaaccccgtctctactaaaaatacaaaaattagcccagcgtggtggtgcgtgcctgtaatcacagctacttgggaagctgaggtgggagaattacttgaacctggtaggtggaggttgcagtgagccaagatctcgccattgcactccagcctgggtggcaagagcaaaactccatctcaaaaaaaaaaaaaaaaaaaaaaagctgcggtgtgtggtggctcatgcctgtaatcctagcccttcgggaggctgaggtgggcaggtcacctgaggtcaggagttcaaggccaacatggtgaaaccctggctgggaaaaaaaaaagaaaaagaatagagctgagtggtggagcagtgtcccaggataattgggaaatgagcctttcctctctggtacctgggcttggcacaggagtcctggtcataccctgcagctgcctctgcagagagccacagggcattagggatattccttgtaatcatcccacgtaatgtttgtgctgcatggaagggcatgtaaaggagtgttctctgcacggtaattgactcattctgtcatcatgattagttagggggcgcccagagcaggatgatggtcagaggttcctcatccccattttaccgatgaggaactggggcttagagaggccacacggctacaaagtagttttatctggatcttcccctctattaagcaagggctggcagagctgggtgtgctactgaaggagcagacatgtgtgggcttggtgccatggaagtggccacaggcagagcagtagcacactccagtctgggctcagcatggcatcaggtacttgtttacatgttgttgtctccctctgaaccgagtttcctatttgtttatctttgtatccagtacacagtaggtgctcaatagatttttttagtgcacccctgtgtctggccttgtgacatagagacctcagaggtggacagggctgaggtgtgctgctcgtgggtgtgaggtctggcccaccctgcacctccagaagacaatgaggccccagccaggtggacaggctgtggacaaggggctgcagccctggcacgagggcctgggaaccagatgggcaccaccgttgtcagctatttagcatttctggaacacctctgtgtgcaagtacccaggagctgtgggggactcagaggcgccaatgagacacggcccctcccctccaggagagtggcctttcaggaggatggaaggctttcctcggtaattatgacacgacctaagcagtcaatggttacttgtcggatattgttgaataattgaaataaagggctgatgagccttgggaggtgactgagatcagaaaaagtagggggctttggagggctctgggcccatgcgcatctacagtgattcccagcccactgcctgacccctcacgctgggatcagcctctgcaggttggcctgggcacgggtgtgtgtgcaagcatatgtgagtgtctgtgtggcattggggtacgcaagcgtgtggctgtagctctgcagagcctggtgctcaggcaacccaggacagggctctgcctcttgcagaagccaattcgtttcactgaattccatgaccacagacagtccctgggaggccagccagccagctggcaaaggctgggcatgcttcttgtcccagggggcccgggcaagtagctggctagatccggcaagttcatcccctcgactcaagcaccaactcgagacatctgctcaaccgatgctgggtcagtagcatccggtttaagtactgaagggaatacacgtatgtcaggctggctgtctctaggtgagggcctcaagagacataaatctcttgtggggaggggtcctgtgtctgggcacagccagcccagagtgtctgtgcctgtgaatgccagtgtgccttgaccttggattgcctgtatctttatggtgtcactgtgtgtgtgcctgttgacatggtagggtatagtggcatgttgcagtagaaagaccgatggactggtaaacaaaagatgtaaattttggttctaaaggcctcctttcctggctctctgatccctgcaagtcagataatctctctgagcttcaggtgtttcatctgtgtaacagaaaaaaataataccagctctgctttctgtaaagtgtaagaatacaatgaaataacatatgtgaagatgctttgttaattaccacgttcagtgcaaatgtgagagattattatcgtcatagcccagcgcatcagaatgagacgttgtatatggcttaaaataatccatccagtgagcagtgttgaaatgtaggtcagatcgtagcctcagatgtttagagagaggaaccacctaatctcacaggccaccaaatgtgaaggtgctttgaaagctgcaaagaatgacataaatgcaaagtgacaattatgtttaagtctagaagggacgttttagtcctcttgtttcctaatctggaatcgtaaagatccgagaggcactggaacctcgtacacagttgtatttgtacgggtttgtagatttgtacgtgtgtctctgtttgcgtctatggactcacgtcaggcacctactctgtgacagtcattcatcaagtgcctgccttggactaaatgcatccgtctaggcattctggatgccagcatgaatcagacacccacacttccctcaaggagctgaccgttgccttgctgggtaggggaactataggagaggaagggtctttcagctcagtctaggcaggggatgggaggagggggcctaggggcagggaatccaggataggaggctggaaaagcgagtaacagtttgctgataaacctgaggggaaggtggtgtggtcagaagaacagtctatgcctaggggctgaggccggaagcagtttgctttcgcgcttggtgctgtctgttgggaccacagtgtggagaagagaccaaggcgatccagccaaatcctggggaccgccctgcacatgagctgaggagcttggctggatcctcctggcccccgggaacccccgtggcaaaggtttaagctctgcagagtggcataatcagctttgcattttggaaagctcctagagccccagtattgccacatatcttgcggcaagagcgcccgccagatgttgggctctgtgtgatagtgagggatcgctgcatatggtgggcggcgtgctgggatttgcacgaggcagggagagggggatggggaggatggagtgggagtgaagtacgaatcaggatgtgtggagcgggggaaggtagcagttctcgagtgactatcacgtgtcagcttttgctacatcacctcatttaattcttataacatcactgtgaagtagatatcatcccattttatagatgaggaaagcaagactcagggaggttaaagagcttgaggttactcctaagcaaagagcagagcgaaactcccagcccggctctgccctctccaaagcctacgtgtctggaatttccaccccctcccctctgcgctggaagtgtggacgaattcagtcccctggcttgacagatgaggaaaccaacctgggctcaaatctgttcccgcgaactgtgttactgttcttcagctgtcccatgtcaccacggatcagattcttttctttcagagtattagcaacgcctcctaattcagggctacttgcagatttgataagcatgttttacattcctttgctcaacccatcagcaaagatatggaataaaacagaccccgtgacagatgcgagaatgaaccacttgctacgcttccttggagccattgaggggcacggcccaagccatctgctcacccttcttggcgtgttagccccctccctccgcagagggaacctcaggcaggcccagagcagaagcatcgcttcccgctttgccctaagactccatcgcggaagagttggcagaagagaacatgcttttcacaacgctgctcttagggccaaatgtcaaggggaagggccaggagttgttcagacctggctctcgtggagactccaagcacgacccagaagcatgcggggcctgaatgctgggccagccgtaaagctccttgcaccaggtgtggggtacacacagcacccctgcctggcactccccgccctctctggacctgtcaggacctggagaggtggcctggcaccggggggtccaccccttcctcctgggctgggttgcacggaattgaatccaacagggaaaaaaatgtgccgagcacctacaggccctgcacacgtggtgtaggtgagagatgccagcaggactgcatatggcagaggggtggcctgtcggggcccccaaaggctttctccgcatgcccagtggaagctggtgcccatcgtgaaatgtcaagcgtctttatttttaggtcattgctgtgtggtaactgtggttccctttcactgatcagacgctctggttggctttcacatctctgattagttcagaataaaagaaagtcaaatgactccctctgtcacaaatgcagcttgttaaagaaatagtttaaagcccagaatttatgggcagtaggagtttttccagaatctgccctaggcgctggggattcagcagtaaacaaaacagacaaaggggcagaccctttgtgaagctgtcttctcaaaacctgagaagcagacaggatggacacctccctacccccattttatggatgaggaaactgaggctccgagaggctgctcctgtaactaggaaatggacaagctgggctcatgcccactttgctttttccatgacctcacatgacccctaatgattttacaagtgctgatcaagacggtgcccatttggcactggttgaatcggattagaagccccacctcagcctgaaattcttagcagttgtgtagatccaatcagaaccttcacaaatactaagctgcactggcaatacgtcctctctgtgtgatcctttcttacactcgggggtctggggtcttcagaagatggttcagaagcagcaggcctcccctcccatggggcgtgtgcccttgggcaccctgagccctcatgaaggctcccgcgagtttgctcctcctcccacagcctttctgccctcaccgcccgtctgctgtgctttcctgaaaagagcccctgaaggatctccggcctcggggctggcctctccctccacagacggcacggggctccccagtcccctggcatcctttccagatgcttcctctgctgtgctccttaaattgccctctttggtccttctccagccctcactgccgtgtcactctgcacagcttagtgacagtgctcttgaaggtcagccattctgacccctagccaagcctcctcatcctcagtatctttctcagacagctttggtctccagtgttggcagccctccccttccttgctcccacaacacaccagggtcctcttctgcacctccgagcattctctctctatctcctgtgtgagctctgcctctctccagcttgactgtggccaccacctatgctaagaccgggcacctctgttcaccctccatgtgcatgtctttggaggcctcccagaacttgagccatgatggtctgccctgcagacatgcctcagctggaggatggatggaggggccagggatgctcttcccgctggtcttatgtctacagctggtcacagtaggtttccatttgtgtgtcccactgtcacctaaaaattaatgtggcaaaccacaaggtattttgcaatcacagaaaatgaaagttgagtaggttgttgagggccatcttactcaggtctccaggagagctgtggaccctcttctacctctgtgctgccagcactgccctgggttggcccctccttggggagggcccaaaagccatctttggctcctgacatcgtgcagggtatgaagagcagcctgtggcccatctgcagggtcaggacgtctttctccccacttccccagcatcatttctgctggggattagtcatgccagagcccagctgagtcagaagggacttgtgggtggtttgtggccaggctggagtagtgaggcaggactttctaggtttatgtctctgtgccccaggagacaagcatgtcagggcccagccgtgcacaggtgttatgtgaggggcggggagtaaaacaaacttctgttccaaactcggtgaatgcaagagatggagaggagctgagattcggtccctgagatcctcctgcaggctgacaggcatgcgcggtgccagctccacatggcaggggtgttcacagctgtacaagtcctccactttccatagttgtccatttcgagtattgcctggacttcaaatagatattgctaaatttaatatttcttttcacttgattttctttttttttttttgagacagtctcgctctgtctcccaggctagagtgcagtggcgtgatcttggctcactgcaacctctgcctcccaggttcaagcaattctcctgcctcagcttccctagtagctgagactacaggcgcgtgccaccatgcccggctaattttttgtatttttagtagagatggggtttcatcgtgttagcaggatggtctcaatctcctgacctcatgatccacctgcctcagcctcccaaagtgctaggatcataggcgtgagccaccatgcccagcgattttattttcaataaaggtgattcattaagtagatcacaaatgtaattgtgtatctgtgaacaaaagctttctcatataaataaacttttaactcaaatcctttatcagagcttgtgttatgattttatggttcggaaaatgtggtcataaagagagagagatgccttccggggggacagcagcgtctgccctgaagttccccaaaggatgtattatttatatatatatatatatatatatatatatatatatatatatatatatatatatatattatattatttagatatataaatatatatctaaatatatatctaaataatatattatttagatatctaaataatatattatttagatatctaaataatatattatttagatatatatatgggtgtgggtatttttatagagatggagtctcactatgttgcccaggctggtctcaaactcctggcctcaagggatcctcctgccttggcttctcaaagtgctgggattacaggcatgagccacggtgcctggccaaggatatattttaagtggggctctgaggggtgcctcaagcctcatcaggcaggaggggagttggtgggggcatattacacagccctggaagtgtggctcgtgggacaggaggaggctggggctggaagggaagccagggccagccccagacaggcctggaatgctacactgctgggatccacctctggcctcacaggctgtgtctgtgcctgcaggacggagaggagcaggaggagccgcgtggcaaggaggagcgccaagagcccagcaccacggcacggaaggtggggcggcctgggaggaagcgcaagcaccccccggtgagtgtcacatcctacacaccctcctgtgcacacacacaacacacacgtgcctggcctgtcgatggcagtgatgtatgtcctgcagggacctggagacatttaagaccaggtgatggggtatacccctccagctgcaggtaccccattcttcaaatcccaccctcacatttccctgttttcttatttatttatttacttttttactgagacggagtctcactctgttgcccaggctggagtgcagtggcgtgatctcggcttactgcaacctccacctcctgggttcaaacagttctcccacctcagcctcccaagtagctgtgattacaggcacgcgccaccattcccggctaatttttgtatttttagtagaggcgagatttcgccatgttggccaggctggtgtcgaactcctgacctcaagtgatccgcccacttcagcctcccaaagtgctgggattacaggcatgagccaccgcgcccggccacatttccctgttttctgcatagagaagtggaggccctgagaagccaggggacttggctggttggtcttgggcctaagacttggaccaggcccctgactcctggcctccctctggaagggaagcaagagggcctgccagaatcccaggctggactaagatctccctggactatctatgaaggcccctccctaccacactctcacccagggtctcagcccccacctggaggaagggaactgaggcctgggagtctgatcaaatgggaagggggagatgcaggctgaggagtctctgtgaggcagcaggagaaccagggccctggggaaggaagtcctgcaggcatgtaggctgcagggccagccccagcgccaggcttctgggaaccagtgccgctgaggcctgggggcagctctggagaacaagacccctgggctgtaggagcctaactggtttatgtcttaaactcagatgtggagcagtcattatttcccagcaccagagacagctggttcctttcttttaccccccttccagctgagaatggtaaacttggagcaagaaggtaagggggagtcagaggctctggatttgttactcagcccttcaaagaaatgttctcagcaggcatggagcccaggacttgctccctttgggtagagagccgggttgaaggtgactgaagtgaaatgggacagtagaggcggggggggtggtgagttcctggaggtggggggtgtgggaacctgctgtgtactgagatgcacccctgccagttctgcctgaagatttgaggcggggggcaggggggcggagtgaagtcattttactggtaagtaattttaaaccttttaatattaaagcaaacgtggatatgtaatgaatgaaattcattctggaatgaaaaattcacgtgatgttgaaaaataacacggggcttcagagaggactttctggctggcagcagactccagattcccagggcccctgcaccctcctctgcccacagggcaccttaatggagaaggtgtgggaggagagccaggccggagtcagagcacactggtgactccacatttgcagcgtgccctgcctctctcctgaggcttggcaacgtgcaatatgctaagcaaactccccctgtccccgtccagtttctgaggacaagagccaccacctgtagcaaataaagacccagcaaccctttgactcatctttgtgagtctctggaatcagagggtagccacatcgctgagaggtggagtgaagcactcgggtgaaaaggtacaaggaagtcagggacaggagtgtggggacatcacctagacaatgacagagaagaggggcacagccgagtgaggggagaggggccggcagtcctacatcccctggcctgaagcacgctccagggcagaaggaaaaacactgtctttggggtccaagagacctgagttcaaattctggctccaccactgaccacctgtgtaaccttgaactgctgctgcctgaacctcaggttccccttctaaaaatagaggagaaaaggatgcatttctccttgcccctgtgagaacgaaatggtgcaagcaccaaggagcctcagcaaaggtcgggcctgcccccgcctggccaaacctttcctcttcaggaggccacggcaaccgtagtttgacagaagagcagcaccttgatttaatgcttcccagcatgtgtccttgagcaagtcacctaacctctctgggctgcttcctcattgggaaaatatggctgccagtaaaacctgccctgtccacctcctggggcacttggcaaacagcaaaagagtccaaatgtgcaggctgggccaggcgcagtggctcatgcctgtaatcccagcaatttaggaagccaaggtgggcggatcacctgaggtcaggagtttgagaccagcctggccaacatggtgaaaccttgtctctacaaaaatacaaaaattagccgggcatgatggcgggtgcctgtaatcccagttactcgggaggctgaggcaagagaatcgcttgaacccggaaggggaaggttgcagtgagccaagattgtgccactgcactccagcctgggcaacagagcgagactctgtctcaaaaaaaaaaaaaaaaaaaaacaatgcagagctggctgtgtaaaaaacctgttccactgcagggcccagtgtccaccaggctggggtgcaggcctatggggtgggggcccagcatcagcctctcagcagccctgggaggcggggcgcatcccgtgcccctcgtggtctggatgtgttctagcccaagtcctaggttacacctgccgtcgcctggcctctcaggagaggcccagggtgaggaggagcatggtaaaggtgaagctgattgggaagtcagctgttgggaaagcaactccttgcacattggaggaaccgagaaagactgaccccgaggacagcagccagcatggcccttcctgggagcccatgttgggggattcctgctgcagccaaggctcagcccttgtggtcgcaggtgctggtcctggcctcttcccctcccatgcaggagcacaggagagatggcttctgaggacctgttgcagctgtggccctgggaatagatttgccagggagctttaaagcagctgagtgtgtcatccagctaagcctggggaaggagcttggctcaggtcctgacaggtgtgacagggatggggactgggaagtaagagatgaaaccctggctggaggctgtgagcttccacagccagcgctggacagggagggtccagatatacccactagtgccctcaccaggtcccacccccaagcccaagaaatcctgggctctctcctccttttgcccccaaggactgtctgtcttggccttcaacaggcatctgtccctgggtgccttctgccccctggcattgccctcacccaaaccagactccacctgccccttgcatgcccaggccagtgtgctcccttcacgacaaacactttccacttcctgagcactgaccatgtgcgggaagccaccagaggaagatgccttctatttcagtcaagtgtgctggagagaggaggccacacataatcacagtaaatgtagcaccggcacgtccttcttcaggaaccccccgggactccattttttaggggacaagatctgaattccagcccctcaccatctgaccactcctttccctgctatgcctgatgccacccctttggccacagaaacctcaccttctcactgctccccaaacacactagccagccattcctcctgcccgccccttacacagttccacagcctgccttctctccgcctgccccagcaccacagcccggtagaacctagcctgaaaatgaagcccagttcagccactgtgacccacactggcctttcccttctccaactgtctccaaagttaataaaaatgaactgataaagacaagttccaagcccaccgatcacacacatgggactgcagtggtctgcagacctcactttgagaagcatttgttgtcacctgtcggtacagaactgagtataactattcagaagcttttatgtgtgtgtgacaatgttgccacatccacgtgctgtgattttatattttacaaccgtatcatttcttggtgcactgagaatttaaaaatctggaaccaaagcttgagctttcatccctgacagtttgaaaatgcaccgccattgcatagtcgctgagctgtgccacactgagtttgcctgcctcactgaggacagagaccagcagtaaaggctcatggttatgcagcatgttactgcctacaaaggcgttttacaggggttaccttatttgatgctgacaaacctgcgtgaggtagttggggaccgctttcattatccccacattcagaagaagagactgaggttcagagaagatgatcaggacttggacttcacacctcccaactgcaaagcctatggtgctctttcctccttatcactgcatccgtgcagtttggctactgggtacctacccaggggttctctgtcagttaatatgtgaattcagggctgctggaaaaagtgtgtggaactggtccaaaaagcatacaaagaaaatggaactgaagagagttattgacacaaataccagggagcctgtgtgtaagtgccgaaggctgcagggggctgagggcaaagaggtgcgcctgcttaaaatcggcaacggcctagttgtctgcagcactggccagagagccaggatgctgcagtcagagccggcaacaggaaacaggaagtcagctgcagggcagggcggggcttaggaagggagctggcacctgggccagggcctgcgggctggccagtgcccatgatagggaggcgggggctggtgcgtggagctgagcccgtgtgtgggtgtgaggtggggaggaaggggcagcctctaaggatgttgcagggtgacaagtgtgggtgtctgaggctggttcctgcctggttgccagcccagacagtcctgcagaggcctttgtccttcatgggtggttccggttggagccctgaagggagagtttctgcctttgcccacaaggccctggggggtgagtggccctgtggggctccatcccctatttactagcgatgcgtatttattcattgacgatttatacccaccaacttcctccagggttatttttgctcagagggagcactctgaggtcacctcctgctaagaataacccagaatgggagggaggaggaggagggggctgggcgctggtgctgagctcagctgagcccaagctggtgttagggctgaccctccaacaccgcaggagtgagccagtccaagagggcaaactgccccacttcagagcagatgacttggggtaacctaaatggggaagtcagggaaccattctgctgcaggcaaaagtcagctaggtacagcttatggcagctcttggaggtttgcagcactggctagctttccagcctgataggacctttgattcttctccaagttaagacagagctcagcagccctgcttgttttcagatcagagccgctcctgggctaaccctccggacctcggctgggtgatatggtgcccctaccttttatgcatataacttcctgacatcgtgtctcccactcctgggctgcagcccttggcccacccctagaggtggcttttcttcctgcgagcctgcatcagtacacgctgctgcccactctgtctcctcatgttttggaaggcatgaggaagacaggccgacacactgcttgtgaggtagagcctatggttatgaaccttgcagctccacccacccactccccgacctgagcggtgaaatcaacgcccgctactggagtgctgcatggaagggcctagcccatggtggggacttaagtgcagtgggactcctaaggatgccggggaagccagggaagatggaggaggtggggcttgacctgggccttagaagggaggtgacatttggagaagtcaaggggaaagtgagaggcacccccgaccccagctgagctgggtgtggaggaaggaaggcatgcagggcagggtgagtgtggggaggcccaaaggcaggggccatgagcacacaggcacaggcctggctgaggggactagggattcattgaaaaatgtatacggggctgggtgcaatggctcacacctgtaatcccaccactttgagaggctgaggcaggtggatcacttgaggccaggaattcgagaccagcctggccaacatggcaaaaccccatctctactaaaaatagaaaaaagccgaatgtggtggcacatacctgtggtcccagctgcccaggaggctgaggcacgagaatcgcttgaacctgggcaggcagaggttgcagtgagccgagattgtgccactgcactccagcctgggctacagagggagactctgtctcaaaaaaataaatttttatatatatatataataaatagtagctgggtatggtggtacacctgtggtcgcagctacttgggaggctaaggcaggaggatctcttgaccccgggaggttgaggctgcagtgagccataattgtgtcactgcactccagctgggtgacagagcaaaaccctgtctcaaagaaagaagaaaaatcttctcaatcttgattggaaaggtagtttgcaggcagcctgaggggcttttactctttatcctctctgaaaaatgtgttatctggaaggttgtgaggaggcggatatgacgtgatgggagtcgtgtttgagaaattggctctggcacccatgagcatggcgttcatccctgagcatgagcacatgctgggagaggggagggggcaggggcgccagcaaagtcccagggcctgtggggggtggcagtggcggggcagaattggccaagcctatgctgctggccctgggaacgctggagaggaaggggtgaaaggaagccctagcaatggcacaacagggacagcatcagcgttgttgtcagagacaaggcttctggaagggagcagatggggaaggggcccctgtcaaggccaccttacatagctggtcttacttggggagcatttaagtagaatctcagaatctcctttttttttttttccaaaaataatccctctgtttattttcatctctccacatcccagggccaccttcagcgagtcattcattcccacaggtcctggccttggtggccttcctggggccccatgagcttttctagggcccgggctccacctgcattgttcctcctgttgcaggtacgcgccggcacacccattggaggagagctgttgacatgggctttctttcatcatccccacctagagaattttgtacattttggttctcctacctcaagaaagacataagcgagttggaaaagctccagagaagggcactaaattgatcaaggggaaggcaaggctgccaagagaggacaggctaaagagattaggactccccagtctgcagagacaaaggcagaggatgtgattaaggctatgaaatcatgtcgggtgcggcgggctgagcacagcacagctggggggcagagggaggacacccattagaaatttcagaaagataaatgaagcacagaaaagatagcaccattttccatggtgggttcccttcttaccaaacctgtcaccctaggattggcagaaattgaaaacagaagcaacttcaaaagagatttacttaacgtatgggatctctcatacgtaatggctttttttgtttttgtttttgtttttgtttttgagacagattctcactctgttgcccaggccggagtgcagtggtgcgatctcggctcactgcaacctttgcctcccgggttcaaacaattctcctgtctcagttttctgagtagctgggattacaggcgtgcaccaccatgcctggctaatttttttttttaagggtcgaagggatttattgaaaatgaaagtacactacacaatgtgagagcaggcctgagcatagggggctcaaaggccattttttttgtatttttagtagagatggggtttcaccatgttggccaggctggtctcgaactcccaacctcagatgatccgcccgcctcggcctcccaaagtgctgggattacaggcgtgagccactgaacccagccagcataatggctttttaaagggagcctccagctcactctgaaatcctttgaggttgacttcaaagagcacaaccaggcccttttccaggtggaccagtgtccccgctggaaacagaaccctgagtgggccggccactcaccacgcatggtcattctggcatccatttgccatggcttgtgacggcctagccaggccatggactgagtgacctctaaggctttgcagagcccagagttctctcctcagacacacgggtggagagatctctggggagaagggagacaggcagctcccagcagatgtggtaaggggaatggtaggacctggagggggtgcccaggctgggaccagagtttccagagtcggccctctgtggcctctggtcccgccatccctgaccccactggctgcgcttttagccaccccacagggaaaagggaaattggaggagtcagggtcctagctccatagcgttggagcttgctccatccaccccaggaaggccgagcaagggggcatctggggaccccctggcccctcctggagtgggcatggagcaggcatctctggctctccctaccacaggccacttccagctgctatcagatctctggtgagggccgccaggggaaagggggctgccggaagggcgggggcgggcagtcatggccccgtgccaatgcttcgggggcagagctgccctgcttacgtggacatgcagctgtttcctgaaactagagtcaacctggaaaaattcaggctggttgaggggtgatgggcctgcagagctgaggaactggaaggagcgaggagttgcggtgggagtggaggggctcactggcgtgtttgtgttggcatcacgcagcttccttgcacccctctgtcctgtggctggcagtgggtgtctgcaggcgtgggggtgcctccttctcaagtctgtgcacgtgacggtgaagagtccgtggctggcctgatgccagggttcccggttggcatggaatggatctcacagaggcattaaaagcaccatggcaggcccggccctctgggtgaccaaggtgaggtcgcccctgcatctgctgggctttgggggagaaggcaagaggagctcccagtgggaaaggaagaggcaggggaggagggagaatggcatctacccaccccccacccccacaggatcccccttccccggtgatcgctgcaggtgggctgcagcctggggacagggcttcttgccccaggtgccagggcctcatctcccctgtgccatgaccttcacctctcctcctcctggattttagtttttctgtgtaaataactatagtaaagcaacacttaaaaaccacttctggccgggcgtggtggctcacgcctgtaatctcagtactttgggaggccgaggtgggtggattacctgaggttgggagttcgagaccagcatggccaacatggcgaaaccccatctctactaaaaatacaaaaattagctggatgtggtggcacacgcctgtaatcccagctactggttgggactgaggcaggagaatcgcttgaacccgggaggcagacgttgtaatgagacgagatcacaccattgcactccagcctggagaacagagtgagactgcgtctcaaaaaaaaaaaaaaaaaatcacttccataaaattagctggatgtggtggcacatgccagtggtcccagctccttgggaggctgagataggaaaatcgttgaatttgaggctgcagggagctatggtcacacccctgcactccagcctgggcagcagagtgagaccttgtctcaaaaaaaaaattttttttaattacttccataaaaataaaaagttaggtgtggtggcaggcacctgtagtccctggtaaaactgaggcgggaagattgcttgagcccaggaattcagggccacagtgagccgtgatcgtgccaccgcactccaacctgagtgacaaagggatatcctgtctcttaaaaaaaagaaagaaaaaatcacctccccatattgggagtttcagacattgccccaccctgggacgcaaggctttgcctggagtgaaggaggggcaggaggtctgggtctggcccagactttgggcaaagggggaagaggaggcggcggcagggctggggaattggttccggctgggcaggccacagcagagggctcctccttgccttgcctctgtcttttgcttgaattgagcgctgctcaggggagggccagcagcctctgcccggcctttcttgggcactgcactgccacctctgccttctccaaaccctgggggcatttgtgagcttgcgcttttctgagactcaacccagagcgggagtcggccttcaaatgcctcgcctgtacctgagccatcctggaagctttactgatcagaggcgtgtgacccaaggcaggcctcagaaaagcgagtatcgctccagctcccctaggatccctgtgaaaattacatttattaattttataagcatatgtatgcccagtcctgtgtgggatgctagagagccataggtggagaggcctacaaagttaaataagattggccgggtgcggtggctcacgcctgtaatcccagcactttgggaggctgaggcggatggatcacctgggatcaggagtttgagaccagcctggccaacatggagaaaccccatctctaaaaatacaaaaattagccaggtgtggtggcaggcacctgtaatcccagctacttgggaggctgaggcaggagagtcgcttgaatccgggaggtggaggttgcagtgagccgagatcgcgccactgcactcgcctgggcaacagagcgagactcggtctcaaaaaaaaaaaaaaaagttaaataaggcaaggctcacagtcgaggagggaggccacagtaaacagataattagattcagaggtatgaattgtaaggagattgggtgagaaagggctttgtaaacaaacgtggcatgcattggctgggacaggagtgacagctgcggtgggttcacccccacccttcagggcaaggctgggccagcagctgtccacacctgccaactacagggggctgaggggtccagatccttccatttaaaggagcattcctttaaaatgctccttagaagacagaaagtagaagagtggtgactgggggagaggcaactgggaagttagtgttgaatggagacagtttccgtttgggatcgtggaaaagttctggagatggatgggggtaaaggttgtataaccatgagagtgtacttaaagtatgcttgaaaatggttaaaatggtaaatgttatgtatgttttaccccaggtttttagaagtttaaaaaagtgtttctaggccgggcacagtggcttacgcctgtaatcccagcactttgggaggctgaggtgggtggatcatgaagtcagaagttcaagaccagcctggccaagatggtgaaatcccatctctactaaaaatacaaaagttagccaggcatggtggcagtcagctgagataacaccactgcactccagcctggacaacagagcgagactccgtctcaaaaaaaaaagtgagcgatcttggctcactgcaagctctgcctcctaggttcacaccattctcctgctgcctccccagcagctgggactacaggcgcctgctaccacgcctggcgaattttttgtatttttagtagattcggggtttcaccatgttagccaggatggtctcaatctgctgacctcatgatccacccgcctcagccgcccaaagtgctgggattacaggcgtaagccaccatgcctggccaaaaaaagtgtttcttaagccaggcatggtggctcatgcctgtaatctcagcactttgggaggctgaggtgggtggatcacctgaggtcaggagtttgaatccagcctggacaacatggtgaaaccccatctttaccaaaaatacaaaaagttagccaagtgtaatagcagacacctgtaatcccagctacttgggaggctgaggcaggagaatcgcttgaacccaggaggtggaggttgtagtgagccgagattgcaccactgtgctccagactgggcaacaagagcgaaactccatctcaaaaaaaaaaaagtgttcctttgataaagacagtccctgtccttgcccctaccctcagcccagggtttttcaaagacattattgccattttgtctggactaaatctctgttgggggcgggggtgtcctctgtctggtggggtgtttagcagcttccctagcctctactcactagatgccagcacctcaccctaaccttgtggcagccaaaaatgtcttcacacacatgccaaaatgtcctggtggggagttagcaaaatcgccccagttgagaactacggccttaacctgcctcaaaaagctgctcacaggagaggactgaggaaaagccggaggcaaggtgggggttctgcccagaaggaaagcgaagattggcaaactcggagggaccccaggaagtggtgggcaggatttgaagacccaaagtgtggtataccgggtgggggtaggtggacagtgggtgataggaccaagctgtactgaaatctagaatgatactagattcacaaccctcctgtggcccccatctggccccccacgggaggagaagtgatgggtagttactgcggcttttcccattctgaaattgtcacctgcagctgcacctgctggtggtgccttctagaatggtcggcacccccaacccagccctctcccagataatgggcaacatgagtatctcgggggtgtgctgtgtctctttcaagatgactgtcaaaagcaaacaaaagcagattgtttaacagacatggcagcaaagctgaaactccctctagcttgagtgcaggaggttctcacttttagcctcgtctttgccattcacgaatcaattgtgaatattgtctgagcacctgtcaggtgccccactccctgctaagtctagccggatcgagcaaagtctctgccctccagatcgagcaaagtctctgccctccagaggtcaacaagctgtggagacaggggaaaggaggggaggagagtggctgggaggaggacatggagttgggggcagaagaggccaggcctggctggggtgcccctccctcctgggcttccaggctgaggtttctgcccttctcaccagggatggctccttcagtgagcacccagctcttccccagagacttggacagttgcttcaggccatttgactgggacctttttccttcctctggatggttttgtcctgctggtcaccctgagccccagagagccccattgaggggccacacttagcttgagggagttcttgatgggtaggatgaatcaagggttgagcgtggcagaagtcattgccctcccccccaaatcccagaactttggctgaatcatctcatccagaacctgagagagcaggtgagcaggaggggatggttgggcagaggatggtgtggaccagatgcgggagtggggatttcagcgggtgagctaacccggggctggaaatgtgcaacatgccccgctcgcccatcatctgcccagcatgcccggctcccacctggctttgggggcatctctgggagaatggcgcatctggtgctgttgcagactggggtccgcgggcaaggccagggcagggcttactcttgtgcatgaaggactgggttggtcgggagcctggggctgtgttgggcctcagagccggccctcataggtctctatgctacggagccagcagtcaacaggaagtgagtcccagcatgagaggaggccatgtggtcccaggcaggaagtgagtgacagtggagttgcctccagcagctttgcggggctcagagcctgccccaatctgaggtgtctgcagggcagcaaatgatgagaagaaaggaagacgcagccatgaaaaagtctggctctgacctaagcttcttagctcaagttagccaggcaggaccccacgcatacctctatggtagtgccatgttgggggctttggggtacctggaacagctggatcccagaaggtggctgcagtgcaggggtacactccacttttcaggctgctggggctcatgactgagccttgaagggtgacagacagggtttacaccagggaggaccattctaggctggaaatgccctctttgcttaagcagggagggaggagaggtgttctgagttctggggagggtcctgacacacaggatggaggcaggggtctgccgttccactttccttcccggatttggccttcctggctctgctggcatcagcccaagcccccaagcacagtggaggacttctcgcttttaccagtcacctcctcttcctctcgttgcagccccctgcctccaactgctgaggagtacacacatgcagctttctgtgggtgcttccctcctggaagcaggcctgcccagctgccccgcacagcaggaaaggagcttggcaggccctgcagttccttctgctccctctccggctgctccctgtgcccgggtccccatccagggaggaaagcatacctgagaagactctggttttgtttttgtttttgttttcccaaggcccttgtccctaattgccacctctgggcgttccttgccaaataacagtgggttgagagttcaggtcctagaccgttagagcaaaagcagccttggaaatagggctttgcaggggaggggctggggacccagtgctgtctcaggctgtgcacttgagtggtgggaaactctggagggaatttggcagtatctctgcacatttagctggacaggccctttcacccggcctgaccacttctggaactccgtcccatggaaacacgcagctgtacagagctgtaagtgcaagggtgttcactgcagtgaaaagcaaatcagcccaaatgcccaccatcagggaacggcatgcatacattttggagcatccagatccatctgcactgggatggagggatgcccactctgcttgttaagtgaaaaagcaagttgcagaacattacgactagtatgatacccttttggaaaaaaacatttctggatctgaatgtgtatatgaatacatatgtataaacatgtaacttacacatgattgtcaatgcataggaaaaaatctggaaaggatgctcactgaactgttaataatgggtaccttaggggagagattttgggggtagggagctgaaataggagctttaaccttttagcattatttttttttttttgtatagtcttttttttttttttttttttttttttttttagatggagtctcactctgttgcccaggctggagtgcagtggagcagtttcagctcactgcaacctctacctcctggattcaagtgattctcctgcctcagcctcccaaatagctgagatgacaggcatgcaccaccacacccagctaatttttagtagaaacagggttttgccatgttggccaggctggtctcaaactcctgacctcaggtgatatgtgagggagcctgcctcagcctcccaaagtgctaggattataggcgtaagccaccacgcctggctacagtttgatttttttatgatatatgtgactgacttttataagtagaaaaaaaaagacaagaaagacaaaatctccattggtctagaccagtcatctccacctgacaggtggggaaactgagatgctgagagcagtgccttccttatctgaggcttgccgtgctgatgctgtccctgcctttctactgggtaggtgttctgcatacggagccagcgaggcagagttgctgagtgcagcctggtgcctgtgggtgtggcagagtgccgagccatggggacccctgcctgttcctgtcccacactggacacctccattcttcttgaggtgtcctggcccacttgcctctggatggctgaggactgaatatatatacacagcagagcacacagcaggcacccggctctcctgctcttgctttgtccttgcaaagcctgtgacctggagcaaatcacaaaattccagagagcctcaggttcctcacctgcttcccagggaggctgagaaactctgaggaggtagtaagtgaggagctttgtccactgcgcagtgacacacacagacagccaggattattctttatttgatttttattacctggaaccttagcgagttcagttcttagactctaggggataaagaaatgatttcccttgaagggagaatgagagaccccagtgctcaggaagagttgttgcagagcctgaaaactgtttgccagagccttggctcttctgggtctgagtgcctgaaagaatctggggcggcccaggccaggggagaaggctacgctaggctatgagcctacagaatggttttttgggggtcctccttcgactctcatgcctcaagagaccaagaggaaagctgggagggcactgaccacagaagtcagaccagcctgagtcccagacttgatctggtggcactggaatcttctaagggagaggggggtcttattggattatgggtggcacaaggagcttttttttttttttttttttttgaggcggagtcttgctctgtcgcaaggctggagtgcaatggcgtgatctcggctcaccgcaaactctgcctcccaggttcaagcggttctcctgcctcagcctcccgagtacctgggattacgggcatgcaccaccatccccaactaactttgtatttttagtagagatggggtttctccatgttggccaggctgctcttaaactcccgacctcaagtgattcacctgcctcagcctcccaaagtgctgggattacaggtgtgagccaccacgcctggccggagcatttcttttttaacaggggctgcagagaaggcttggacgtcctatgtaacctcagtgagtggcggtgggatgggggagccgatcccttgagaagtgcaaccaggctggcaaacctgcttggattcttttcctgtttctgaagctggagtccacagactcgtgatttcaaggggtcaagcccaggaatcctttgatgttgaaagcaaaattgtgaatgtatctgtttgcatgaaggtttatatgaacacagtttctggacagaaagtgcataacattctctagaatttcagagcggtcaatgatccaaaacaggctaagcccactgatctaaccctcctcttgtgtatctttcttacatttgcaggtggaaagcggtgacacgccaaaggaccctgcggtgatctccaagtccccatccatggcccaggactcaggcgcctcagagctattacccaatggggacttggagaagcggagtgagccccagccagaggaggggagccctgctggggggcagaagggcggggccccagcagagggagagggtgcagctgagaccctgcctgaagcctcaagagcagtggaaaatggctgctgcacccccaaggagggccgaggagcccctgcagaagcgggtgagtcctcagcaccaggggcagcctcttctgggcccaccagcataccctgagagtcagggacttggctctccagcaggtcccaggaaggatggtctgggtcgtggctaaaggtctgcttgccaaggctatggcctggaggctactggctggatgcagcctgcgcatatgttttatttggcccatagagtgttttaaacatttaaaaaattagttgccagtatttaaaaatcaaaaaatttcacataaaaatctggagttttggcttctcatgaaaaaaaaaaaagctagatctggcaacagcgggctttcataacgccaacgattgctagactgggataatggcggtccctccatcgccttctgtggctggttgtgggccttagttttctgcagctctacctggcctgcttactctcccacgtgccatgcagttcctgggggttgctgtatttgtagcccctggcctgggcactcaagggcagcagataccctgtttgcctccctgagtgcagaggtcctgagcccaccctagttgggctgactcaactggaaatttggttgtgacagtggcgtggggagagggctgggtgattgtattctgtgtactgcccagcccaggcctcttcatctggggactttttggcctaaccctggaagcctggaaagttgcccacttttctctttcaggttaagccagcaatttcagggccaaccgagctgtaaacatgttagtaatgaggacaactagcatttgtacagggcttcacagtttacaaagcgctttctcatacattatcacatttgatcctcccagggccctgccaggttgttttgcatatgtgcattttaatttcaaaaagtcttccttccaagcgtgtatgatgaaatgagtaaattgattaattggcgtaacttattttgcatggatccaacctaatgttcatgcaggatagagaacatttccagaatacaaatttccaaacttattaagaagcttttattcatttattattcgacaaatatgtatgatgctcccactatgtgcagaacactacagagggaatctgtattagttgttttatttactcctctaacaatcttattcaagagatatgattatcctcatttcaggagaaaaatattgaggcttaaaacaatgatgtgacttgctcatggagccccaaccttaagtggcagatccagggctcattcagtcctggtcttgtgcctttctactattttccgctgtcctggaatgggacttggtgcggagacgggtcctgaattgtctaggcctcttgcacatattctgatacttcccgagagtgggaagggcctgaggggagtgaaacagatgaggaaagggagaagacaccatccttcccattaacaatgccaggtgtgtgagagaacccatcagccctgcccttccaaatgtggtgcttcccatgggctgactgcagttgacttcgcttcccaggaccccaccatgactcctgccgagctgctacccagggtcacggcattccttcctgccctgaagcatggagccatagccccaaagctgttctctggccttgagggtttgttttcactttgggatttccctacttccatttctagctgttctcaagggctcctgcatcgatcagtcaacaggtgtttcttaaaacacccattcagggtgtgctaggatccataccagggactggggaaactattatgaacaaaaacctgcttcttgccctcaaggacctcacagtcagctggagagacagacattaatgagaaatccacccttaaaaagatgagattacacatggagataagcgctctaagggaagtgaaacatgtccaggggaactgcaacgaagaacctgagttggcctgggctgtccagcgcgagctgaagggcgaagggagtccactgggtgaggggtagagggagagagtgagaacagggaagaaggctcccaggtgggagggagcagggcacccagggaactgatagatgtcacaaggctagagtgcagagagcaaaggagaagggtgtggaaagtgctggggcagacaggccaggccttcaaaggtggctgccggggtggggggtgggggggcggggaggcggggggtggggaggcggggagtgggcagtgtgtgtgatgtaatcagctttgcaatttgaaaagctcatgtcatctggatcctgggatggaatggaggagctgggaagcaggatgacatcggaggggctgatgagcaggatggcagcagagatgaagggaggggacaaatgccaggaacatgagaggtgagggagagatgctgtcaaggatgattcctgggtctgggactttcagacaggtggatcttccttagaatcttagtgctgagtgtggtgggcaaaaactctttaagtctccttgacctccaaaatttggccttgaggctgggcacagtggctcatgctggtaatcctagcactttgggaggcccaggcgggcagatgacttgaggtcaggagttcgagaccagccgggccaacacagtgaaaccttgtctctactaaaaatacaaaaaaaaattagtcgagcttggtggtgcatgcctgtagtcgcagctactccggagtctgagacaggagaatcacttaaacccgggaggtggaggttgcagtaagccaagattgtgccactgcactccagcctgggcaatagagtgagactccatctcaaaaaaatacaaaaaaaaaaaacaaactttggccttggtacactcctgtgtggtcccagctatccaggaagtatagattctggcagggatcagagctggtaggatccctgaaggccactgcactgcctggaagctgaaattggtgagatgttacctcgcggggtgggcagtggcctaggctctgccaatatgcaacggacagtggcttaatcagggtgtggaggctcggtcacggttccagcagtcagctgacatagaacacatgctcagctggttgattcagatgaggaaggagactaaggccccggagaggatgtggctcggccgtggtctcctttcagttccccttattccaggtctcttttcaggggatgctctcctggaacccctttcattagctcctgtacagcaaaaacagactttttaaggcagcgtttcttgtagcctgagcatgtgctgaaagtacagccaggagaaagaatcaggaagacgggagatgatgagatgagtgatagggacaggagccaacacccgacaggcctgtgcttgtcttgggtctgttcctgcccccgccagcattaatgccataatgatgatgattcaggtggcagtggcgatgtggctttcccttattgagtattgactgtattgggcattctctctcctcgctactcacagtggtccatagaccagcagtgttggcagcacccaggaatttagaaatgcagaattccaggctccactgaagacctgctggatcagaatctgcattttaaagagggcctcatgcttcccatgtatatgaaagtttcagaagctctgctgtgtattatgccccatgtgtgatcacgggaggaagatttttttttttttttgagacagcgtctcattcactctgtcgctcaggctggagtgcagtggcacaatcttggcttactgcaacctccacctcccaggttgaagcgattctcctgcctcagcctccggagtagctgggattacaggcgccagccaccatacctggttaatttttgtaattttagtagagacagggtttcgccatgttggccaggctggtcgtggactcctgatctcaagtgatctgcccaccttggccttccaaagtgctgagattacaggcatgagccactgcactcagcctggaagactcttattatcctcatttttcagatggggagactgaagcttcagaaaggttaaattacttacccaagattatgcaactggtgagagaccggattagggtcagcacaaaacccctctctactctgagcatagggccccttttcaggccagtgactgccctctgaggtaaacctggctacagcctggtcatggagatttgagttcattttaaagagaacttgggtggcctctggctgagtatagctcagactcgaaggcgagtgttagaagcaccctggcctcccctgcccccaagtcctgagatgcactagggtcatgttcgggcctgtcagggccacactggaaggtcacaaccacctggccctggcggtgaccctgggcctgggccaggagcagttgggctgcataggccgtactgagagagcagctgaccctccctgactcacgacaagtgccgcattccgaggcggcccggccaggctggaaagcgtgtgtgtctgtgtgtgtgtgtgtgtgtgtgtgtgtgtgtgtgtgtgatcactcaagtgggcatgcgcagagtctgtcagggagaaaattccagaaagaagccagttggaaactgcatagggaaaaatcgttctgttttggggagcttggggggctgggaaattaggaagggggtcacattgagaaataacagtagtggtgcagcggagagggagcttgccggggcatcaggacccctgaaaagctcatctcagctccggccttaagtcactccaccccccaggtctcagctttctgcaaaatttaagtgctgaacaagtactcaatgatctctgcaaagcctttcaaccctgattttctaatattctaatgaataaataattatccgtaaaagtaattaaatgccatggagggtcccaagtaggaaacgtttgaggttctaaagcgggctggggagaaaatgcagtcggaagccccaagtccaggtccgagagccagcccgggggtcctccactccccgccctagccgcagggctgacacagtggttcccagcgccaccgcgagccccggagccccgccgcgtcagggtccgccgggaccgccaggccgcccccaggccgccacctgatcgcgagcaggcgccccctgctgggagcccggggcctcaccgggcagagcaacgaggtctgggaggagggggaagccctggcctgggaacggggaggccccggacaggctggagggggcccccgcgaaaaaagtggggagctggctgtggtgggagagaggacggaaaggaaacagggaggcagagccagacaagatgggtgagacagcccttaaaaggccggtcacgaacaaagcgctggcgagtgcgcgcccgcccacgcgcacaggtgcccgcgacaagacgccccgtccccgcccacgcggcccccgcgggctgagcccggcgccaggcccgcacccacggtggccctcctgcctccctccccctggtgctcccgggcctagctagcgggagtcgctgtcaccagggacagaaccacggaggacagcgctgtcctgggcgcccggggtcagcccaaaaccagtccaggacccccctgtcacgagggaagggagtggcctcccgggctgaggacccgcggcgtgaacactgtggctggcttccgcttcacagccaggagatgaagtcggggctgttttttccgtttcaggctctttgtctccccccaccccaagctaccatgagccagccttagtgaccttcctgcagtgggcccaggcccgatacgctgtcggttcccgacaaagaagctcaggggcccaggtggaaggagggctcacttccccacctccagcaagaagctatgggaggcagaatcagccagccccgcagcctccggggcgctgggcctggaaggggtgcctggaaagggtggtgaggacctggtggggagtgcagggggatgcctgggaggagcgcagctcccctcatgctgggggcttggagaagcacagggagagggcaaggtctcaatagggaaattgagacttccctgccaggacctggaatcagggcccagtagaggaaatgaatccttagggaaggagaaccacactaacatgtattgagcacctacagtatgccaggccccacagtaggcacttaatgctcaccataacctaatgattttagaacatttatctaccttgattcagtgggaaaacaggccaggagaggttacagaggtcacactggaatccaaactcaagtcttctagattaaaggctgagctcaggggcctgagcatggggcagggagtggagggacggagaatgagattagtaatagaatcataatatttggacctgcaggcggaccttggaaatcgcatccagcatccttattttccagatgaggaaactgagtctcgtagagaggaagttgcttcaccggggtcttgttccagttggcccggtacccaccgccccccagtacatgcccttatgcccagaaggcctctgcaaacactcccttgcagggccagggaggccgctcaggctggccctctggcccaggccttcctgcaccccctcctccttccgcaccccctctgccttcctgctcctcctttggggaaggaggtctcttgactgggagcagcagaatgggggtatttttagtccccttctgtgtacctgagacagagttgacaggccagaaacccagctctgaatttcctcgtgttccctcttgcctgggcctcaaggcctgcccctgcccgagttctttctctgccctaggccctgctgggtcctcagggtcagcactgtgcctggcatgtggtgggccatcatatttaacacatgaacagctaaacggccagaggacccccatcccctgtggcagggaccccaggcacaaggtggggcctgtgagccactccagtaattctgccgaccaacgaactggtccctttgttcttccctctccacaggcaaagaacagaaggagaccaacatcgaatccatgaaaatggaggcaagtgttccacccccaggggctctgtggatcctgattctagaagaaggacacgggtggcgagtgtgtgtgagggggcggagggtgggcccctcctccttctttacacatccctctgtgggaaggagaggaagtgggcgtggaggttggaagtcccccaacaaggcagccggctcatccccctccagaggccagacgcctcccttcgtggcctgtggccgtgtccatgtgcccccccacccccgccacacatgcccctcgctcctccagcccacacggggcctgcccagcactcccaggggtgccctctctggccaggagccagtgtccacattgtgtttgaggcgagtgcctccttggtgggtccgtcagtgcccaccctaatgccctaatgtctgtctctctgtcctagggctcccggggccggctgcggggtggcttgggctgggagtccagcctccgtcagcggcccatgccgaggctcaccttccaggcgggggacccctactacatcagcaagcgcaagcgggacgagtggctggcacgctggaaaagggaggtgaggcgccttctggcctgggccccagcccgtcctgcagaactggaaaaccagaagtgatgggcctcagtttccccttcaggctaacccttagaacttactgggagcctcttaatctctagtcatgatgactggttcatctgctccttcagcagatgcttattgaacacttactgtatacctggctgtgtactagaccctgggcgtatacggttcctgctctcatggagttgacattctgatgggggagacaggcagaacataaagaaacaaggggctgaaatagaagatagcagaggcctgctttagttggggtggtcaagacaggcttcttggaggaggtggcatttcagttaaggtctgaaggaaccgaagagctggctctgtgaggagtggggtcagggagagtgttctaggcagaggaaacagcaccagcaaggccttgacttgtttggcaaactgtatgccagggtgaaatggcaaaggagaaagggccagatgcagccagagaggcaggcggggctccacacatcagttggagacctgcagagtctgcccaaaagctggcttacttctgacaatactgggcagccatagaaggtttctgtgcacaggaaggatgtgatgtggatttgagaggcagcagtggagacagagaagagctttggggtgtattttggagctagaatagctagggcttgctgatggaccggcagggaggggaactggtggactcaaggaggactcacaggggtctggcctgaacacctggtgatggttgtgccatttgctcagatgggaagacccaggggaggatgagtttgggacaggtgggaattgagggctccgtgtgagatgccaggtgcctggagtctgggctgtagggaagtacgggcgacagaggtgggatttgagagcagaaccctggcatttaaatccatgggaacttaagggctcagcaaggggagaaggaggagagacgaagtctcagaacatgtccatgaaaaccccgacatttctgggccacatggagaggaggagtggcaggcagtagggactgagggagaaagcagtacagagcggtgtctggggaagcacggggttttcccagagggtggctgcccagccctagggaaagctgaattcactgagacatgcccatggcgttggacaacctggaaaggagctggtgactttatagagaagcttctgtgggggtcggggaggtgggaacagcccgtggagggcttcagccatgcagagaggtgagaaaatggaggctgaaagactgaggagggaaatcaactcgccacgcacaatttttgagctgtcttgtatgtttgattagtagagaactgggtacaaagagaagtgggtggtgacctagctgcacaggaaaagagaggaaactgccaccacgtggggcacttcacgtgaactcagctctcatttcgtctttaccccaggcctccaggggaaggcggccctgtttgtctgttcgcctttgtgtgagagctttctctagagaaagtgcagaacagaggccaggcgcggtggcttacacctgtaatctcagcactttgggaagctgaaaggaggtcaggagttcgagaccagcctggctagcttggtgaacccctgtctctaccaaaaatacaaaaaattagccaggcgtggtggctcatgcctgtaatcccagcactttgggaggctgaggcgggcggatcacctaaggtcaggagttcgagaccagtctgcccaacatggcaaaaccctgtttctactaaaaagacaaaaaattagccgggcatggtggcaggcgcctgtaatcccagctactcaggagactgaggcaggagaatcacttgaacccgggaggcggaggttgcagtgagctgagatcacaccactacactccagcctggacaacaagagcaaaactctgtctcaaaaaaaaaaaaaatacaaacaattagccaggtgtggtggtgcgtgcctatagtcccagctactcaagaggctgaggcaggagaattgcttgaacccgacaggtggaggttgcggtgaggcgagattgagccactgcactccagcctgggcaacagagcaaaattccgtctcaaaaaaaaaaaaaaaagagaaagtgcagaacagggtgggggttctggctgcttgattgttttcttttcctctgggatccaggaaggcagaaattactttcccggaagagcccggagaaagggcagcctgtctgaggtctgggaggagaggcacttacccctgtgcgtggaatgttgtgtttggggtggaaattcaggaatggggctcttctctccaagtcagtcgctgcactcctgggttccccgcaacccagaaacaggggctttgctttttagctcctggttctgtgagcgaagtgtttctgttctcatttagccctggggcaagaagcttagactatgacttttaggaaccggaagaactttcagaaattccatgattctcaaactctgctcccttgaagaagcagtatggaggcctgccctcagttacagtgaaaaaaagggttctttcctcaactcccaggaaaaattagatcatagatacattttctcaattttaggtctctactaaaatttaaaaagacttttttgggctggacgcagtggctcacacctgtaaccccagcacttcgggaggccgaggcgggtggatcaccttaggtcaggagtttgagaccagcctggccaacatgttgaaaccccatctgtactacaaatacaaaaattagctgggcatagtggcgggcacctgtaatcccagctacttgggaggctgaggcaggagaatcactggaacccaggaggcagaggttgcagtgagccgagattgtgccactgcactccagcctgggtgacagaacgagactctgtctcaaaaaaacaaaacagactttgtaaaactcatgaacattttctaacatatgcagaagcagagagtagtgtaacgacctcccattgccatcacctggctccagcaattatcaaaagattttctagtaagtcttcaacccttggcatttgcatccccactcccaaccaatgaaggtagcagtgggcattattagaaaaaccaaaatgttgatttaatttaaaagactttagtacacttgacttgcaatgtggagatctccaaacaaaatatattttctgctaataacattattccttattcctatgctgtaattaaagaatccatgaattccagtacaagttttagaactactgatctcattttcagactaggggacttggggctaccgagttttataactggccacaagctcccaatgaagaattgggaacaggactagaatggaattagaatttacgtttcctattccaatgtggggcccaggccctgactgtcacacagtctcctcctgggctatgcctggggttaggaaggggctagatgcaggtatcaagaatggggaaaacacaggtagctttcaagattcccaagtcgccctcagactcaagagtagtagctgttgctatcaggcctgttttctttgtttgtctgtttttgagacggagtttcactcttgttgtcaaggctggagtgcaaaggctggagtgcaatggctcactgcagcctccgcctcctgggttgaggcgattctcctgcctcagcctcccgagtggctgggatcacaggcacctgctgacacacccggctaactttttgtatttttagtagagatgggggtttcaccatgttggtcaggctggtcttgaactcttgacctcaggtgatctgcccgccttggcctcccgaagtgctgggattacaggcatgaatcactgcacccggcctatcaggcctgtttttgttttttggtttttgtttttttgagacggagtttcgcacttgttgcccaagctggagtgcaatggcacaatctcagctcactgcaacctccatctcccaggttcaagcgattctcctgcctcagcctcccgagtagctgggattacaggcgtgcaccaccacacccggctaattttttgtaatgttttctaagtggtgtaacaacagcacagacatttctcctcttttctcagtctcattttgctctctgacatttgagcccttctgaaactgcctccctgggccttcccaaggccgctgcaggttcctccccacctccttcagctgatctcaagcggcaacctctgttagctgcatccccagatctccaccccccaccctctgccagtgttgatccactgaccccttacccaccctcctagatccaagtaccctcctcctcccaaaaaaggcacctcccctcctgacaaaccacccagccttcaggacccagctccagctgttccccaggcaatatcacttgaagggccagccagcgcttcctaatctcagaatgcaacgcctgcaacctttcattcagggcacctttactgagccggtttccagaagcctccacagctgagtgacatgtaagatactgggactaccagaggcatagaggattcctagttttcccatgataggatgagggctcatagcacctgcagaaagagctgccctgaatggggcgtagacgaatgctgcctggggtcatctacgcatcagcaggggtggcttctggcttttgacggttgaactggaatttaggggagggctgaggaaggcatttcagagggaggaatacagcaggcacatacctctcgaggcacaagacacttgggcttttcaggaatgggagtttgttgtggtggtgcacagcgggtgtgggcagaaagcagctaggaaatgtggctggaaggtggggcaggggccagaaggaggagctgccaccagaccccaggctgcaccagcccttcccctaggctgtgtgccccacctggggggcttcttccctgggaacgcactccccagcttaacttccttctccttgcccacccttgttggaccacagttactgagtgagcctctcccagattacccccttcaggctttccctcccccaagatttaagacgactcagaatgtcacaaaatgaaagaaatggaaaataagtgaacggggaaaatgagacatttctgcagtccctgaggctgaagtgagaattcctgctggagctgggaaggaagttgttgtcttctgtctgttccatctcctgttgctgttccttcacctgcttactagtcctgtaaggactaagctctgttttaatcatgatcacaccctgccatagaacctccaatggctccccattgtctcccccatcatgtcagataccctctggctatttctcaagtcttcaacccttcgacctccacaatcagcccaccccactttcactacagtcctctaatagaagggtgtcccctaggaagggacattgccatgctccttccttccttcctgtcatcgttcctgctgtgatttccccagtccattcttcagggttgaggccaagagctgctgcctcctccaggaaggtttccaagatttctccagtccacaatgacctcttcccttccctgaatccctgtaaccctcactggggtttgtttgtttgtctgagacagtttcactctgtctcccaggctggggtgcagtggtgcagtctcttggctcactgcaacctccacctcccaggttcaagtgattctcgtgcctcgcctcagccttcccagtagctggaattacaggcacgcaccaccacacccagctaatttttatattttaagtagagatggggttttgctatgttgacctggctggtctcgaactcctgaccccaagtgatccgcctgctttggcctcccaaagtactggaattacaggcatgagccactgcactcagactgtccttacggttaatattacttagcactcaatcatatttcatcagggtatttttcactgtttcttatgtgttgatcacatcttcctctgtctggcctaatgcaatgggcagggagaacaggcccagtgaatacctgctggctggctaagcagttgggaggtaggtgaatgtgggaccacttctctgggaacatgagatcttagatttccaccatgacaagggcccacacacttgatttcgaggtggtttttccaggggtgctgacaggtgtacctggaggatggcatggggactggtatggacagcctccatgggtaaaggggggccagagtctcctccatcctgtattcctgccattctcaccaggcttctgagctgaggagaaccttccttggcctggcagagctggccagggctgtggggagggcagggtagggcagttcctcctcccaaaggaaggcactcccttagctcctcttccttctaattctcagagcaaaatgcactctgatggagggcttgagggttaggaagggcctctcgcttcattagccctccctgtagctctgcaaggcagatgagcacagagacccagggtcagtagctgggcggtcatctcgcctgggaagccactcactccagtttgtgtcgcggctggtggttttaggactcagttacagcacattgatggcatgactcgctcattaatctcttcaccccagccggattctaaattccgtctaggcagcgggtaccatgtcctattgttctaggatctggtctctttcctgcttccgacctgaaggtagggacccttgactcatgcatccttatacttattagatgcttaataatattgaatagtgataatgaaacatctgtgactggaacatcatagtctctcctcccttccctcgccacactgagatacaacccactccctccgcccagggctgcccagcagatagcagagccttcatggaccttaagggttggctgctgtttactgcgacctttggaggcaagttcaaatactgccttaagaaaggtcccacttactgggtgtgggaccttaggcatcacttaccctgtaccccagctgaggttcctcctctgtggatggacttggctgtactctgcatctccatgacagcagcactcgccagcttgttttctgcctctacccatttctgcatttaagctcgttcaatacaggcattctgggcaccccctgcccccagggctggcatgtgggaggtgcagggtgaaagtgtgctgaatgaatgaacaattcttatctcagagttgtgtggacaacacacaacatttaaagctcctagcacagagctagcatataataggaactttataattttttagaaagtattttaaattacataagtattacacaaatacattcttgtgaaaaagtccgcccaaacttgccaccccaaaattttattttgagacagggtcctgctccattggccaggctggagtacaatggcatgatcatggctcactgcagcctcaacttcttggctcaggtgatcctcccacctaagcctcctgagtagctgggaccacaggcatgtgccaccacacttggctaatttttaaattttttgtagagatgggggtctccttatgttgaccaggctggctttgaactcctgggctcaagcagtccttttgcctccacctcccaaagtactgggattacaggcatgagccaccacacctggcctgaaaatttaattccttcaccagaaatacttgatgctatcagtttggtgtaacgtcatgattttttcctatccgtgtgtatgcgtgtaaatatataattttgcttcaggggttctttttttcctgttctttttacacaaatggaaccacactgtagtttcattttacagcttggtttttgacttccaataagtctgtgcaatctagccacgtaattactattatattccaccatgtggattcccatggttttatttagccattctgttattggtggccatttatgttgtttatgatattttactgtttagcattgcagcggtgaacatccttagtcatacctctttgtgcatctgtttctctaagacagatacctagaaagaaaaaggcctagctgaagggaatgtgcattttacattttatgacatctgccaaactgccctctaaaaagggcactccaatttatgcttctgtgaacagggtatgagaagacctttgccaacatcaatattttgctagtcaaatgggtgggaaaatagtatcttgttatgcttttaattggcatttcccagataataaggttgaacatcttttcctatgtttattggccatttgtgctgctgcttctatgagcttagcaaatgttgcctcctctgactttacaaccctgcagtctcaacagactgtgcttcctgtctgccctgaggagagcaggccatgaatgtccagaaggtcccccatgctgtgtcatattccatttaataagcagtcactcagcacctactaagtaaaggggccttgtgctaggggctatgggaaggaggagataataaggtgtgatctcctgggctttagtctagttcaggagaaatggaagtgcaaagtagaccagtaaaggctcaaaacaaattattagccaactgtatgggaagcagttttagagcatttggactggaagacattcctcacttatttgccctacctgaccttttgcagcgtggcctcaaaaataacatcacccttgaaggagatgatgatgtttggagtaggcacagcaagtatactttacccaaggctgaacaagttctagaggctcccatgacagcctgtaagcagtagtgacttcgagggctggcctttaccataggctactaaagttgccacagagggactccccattgtcctttaagtagatgggttggttgcgcaccagagtcttcacttctggggcacagagagaacagctcttagaacaacccctactttttagtggtgagaaaaaaagaggcagggtggggtaggtgactagttgctggcccagggccaactcctggccacagcaaagcctgaaccagggaaagggtgttgaacttcactgagcacctactttacatcaggcactatgccaggagcattagctttcaccacaatcctaaaacatattatctgcatttaatgtcttttttttttttttgagatggagtcttactctgtcacccagtctgaagtgcagtggcatgatctcagctcactgcaacctctgcctcttgggttcaagcgattctcctgccccagcctcccaagtagctaggattacaggcacctgccaccacacccggctaattttttgtatttttaggagagatggggtttcaccatgttggccaggctggtcttgaactcctgacctcaggtgatccacctgcctcagcctccaaagtgctgggattacaggcatgagccaccgcgcccggccagaagtatttttttaacctgtattttgctattagaaaattaagatttatctaggtaagtaggttgcggccacatgggtatcctgatggagtggcccaatgggcaaaactagactccaaatctcgtgttgcttctgttgtgtacccaaatgactaacctacactcccagttttcctggtttcctggcttggaaattgtcatgaaattgaagaatgcatttagaagagagggagagagctggcccggctttccagcctgccgcatgctcttcccgcccccggcctgcagttgttggggctgctccccatgcctctttgccaggaggctcaggtccctttcagcctccccgaccacccctccctgctctactcatgtggtcctgaacccttgcccttgaattgctgttttttcatgaccatgtggatgatgttgtgaaactggattaagaaaatgttggaatgagatttgggaggcctggtttctcgtctcagcttctgcccctccttagctatggatgtgaggaggtctttctgctttcggcattttagtttcctggtttataatgcgacagaaatagatgagatgaccccagagattctaaaattccatcacggcaggttggggggtgagactggaatcatgatctgcactctctttattacctagattaccttgtacataaattctaaattaaatatttgagtgaattctcagacctcgcatatacgtctcttctaaaggcacatacgtgaagcatccttataaaaactctttttggccgggggtggtggctcacgcctgtaatcccagcactttgggaggcgaaggcaggcgtatcatgaggtcagcagatcgagaccatcctggctaacatggtgaaaccccctctctactaaaaatacaaaaaattagccgggcgtggtggcaggcacctcccagctactcgggaggctgaggcaggagaatggcgtgaacccgggaggcggagcttgcagtgagccgagatcgcaccactgcactccagcctgggcgacagagccagactctgtctcacaaaaaaaaacaaaacaaaaaactctttttaaaaaaaaaacctttggcccagcacagtggctcacgcctgtaatcccaacactgtgggaggccaaggcgggtggatctcttgaggccaggaattcgagaccagtctggctaacatggtgaaacgccgtctctactgaaaatacaaaaattagctgggcgtggtggcacatgcctgtaatcccagctacttggaaggctgaggcacaagaatcgtttgagccagggtggtggaggttgcagtgagtcaagattgcgccactgcactccagcctgggcgacagagcgagaccttgtctcaaaaaaaaaaaaaaaaaaaaaaaaaccaaagggttgtccagcctttactgagcagatactgtgtgctcagtctttaagataccaccttgtgtgtctgggtgtctactatgttccaacattttctagacactaaggaagcaaatttaaaggcaagtcagaattctaccctcccctgtgagaggttggtaggtaaatgaatgaattgtgctgtagccagggcagggttgttttagaaatactaagggaaggactgtgcaagcaactccctagccctgcctaagggcctcagggaaggcggtgcctagggagaggcagtgtaccgccattgacctgaacaggactgcttggcagggacagtgggggaagggcatggagtggggtcctaagggaggtggtcaaagaaatcctctaggagtttggagtctagagtgcctcaaagtttttcccatagcactgtctatggggctgacaaagcagaaaatataatagccaatgttctctctgctttaaaaaccagactgcttgtcagggaggccgtgggatgttcacatatggggtggtctgccagagccctctgccgctttattaagaggccagtacactcgttaaaggagttaatggacctcagtacttactactgattgcttagtaattcagtacttggtaaataaacatgatactatgctagtggcggggtagggggagaccaattggatatagtccctgtcccagaggcacacattgtcaagtggaggaggcaggaatgtttccaacaagcctcagtggcagtcagagcgtggggcttgctctgtgctcctgtgtaaagagcccaacatgaagcataaaggagcatgagagcagcttctggaggactccaggaaagcttcctggaggaggcaggcatcctttgagaatttcagtaagtgcaaaagaggctgggtgcagtgtaatcccagcactttgggagaccaaagtgggaggattgcttgagcccaggagttcaagagcagcctgggcaacatggccagacccactctctacaaaaaaaatatttaattagctgggcatggtgttgtgcaccaatggtcccagctactcaggaggctgaggtcgaggctgcagtgagccatgatcatgccactggactccagcctgggtgacagagtgagatactatctcaataaattataaacaaacaaataaaaaagtgttaaagaggaggaaggcatttcaggaaaaggaactactgagggcaaagagaaggaggcatgacttctgggttgtttagggagtagtaagagatctggtaactagggtgcaggggttagggaggatgggcaagaagagacagaactagaaagcaagatggtcacatcctcagtggtcttcctgccatagtgagaaatttaggcttcgttgtccagtggagagcaggggtggaagagaattggacacatagtttaggaagataacctggcaggacaataagtattccccagagagaggaagtttagaagcaggaccaggtaggagaacattacatttggccttgactgagggttgaggtaatgagaggcaaacttgaaggaattgctagaactggtcctggttggaggggagggaaaaatttgtctggtattgggagctgggctggggatagggttcattatctgaggtagagaatcttggaggaagagcaggtttgaggcaggatgatacagtgaacttttcattttcttatgtattctgatgagcagaagtttttaataaagtccaacttacccatttttttcctttattggttagtgtttgtgtgtgttatctctaagaattctctgtctaccgcaaggtctttaagatattttctcttaggagggggtggatttttgagctagggtttggataagctgcttttgagaagccagcagattggtgtcaatggggacaggttttttttttttttttttttgagacggagtcttgctctctcgccaggctggagtgcagtggcacaatctcagctcactgcaacccttgactccctggttcaagtgattctcctgcctcagcctcccgagtaggtggggttacaggcatgtgctgccatgcccagctaatttttgtatttttagtagagacggggtggggtttcaccatgttggccaggatggtcttgatctcctgatctcatgatctgcccaccttggcctcccaaagtgctgggattacaggtgtgagccactgcgcccgaccccaggggacaggtttagtcagattccagggtggtcctcatggaggggacgcacaggcaggagggcagggagagaaagcacagaaagcagacctgctaggagaggaccaggatagggaggggcagggacaaggacatgggacttctgggagcaggtggccgggggtcaacgtccaggaggatttcctgaaccagttagaggactactgaacccccctttcacctatctatgcagccctttacttttacaatatatacatttctatttattttatttatttatggtttttttttttttttgagacggagtctcactctgtcacccaggctggagtgcagaggtgtgatctcggctcacttcaacctccgcctcctgggttcaagcaatcctcccacctcagcctcccaagtagctgggattataggtgtccgcccccacctctggcttttttttttttttttttttttgagacagagtctcactctgtcacccaggctggagtgcagtggcatggtcttggctcactgcaacctctgcctcccgggttcaagtgattctcctgcctcagcctcctgagtagctgggactataggcacacgccaccatgcctggctaatttttgtaattttagtggagacggggtttcaccatgttggccaggctagtctcaaactcctgagctcagatgatccacccacctcagcctcccaaagtgctgggattacaggcatgagccaccacacttggccacctctggctaatttttgtatttttagtagagatgggttttcaccatgttggccaggctggtctcgaactcctgacctcaagtgatctgcccacctcggcctcccaaagtgctggaattacaggcgtgagccatcatgccgggcctacgttaatttttgagacagagtcttgctctgtcgcccaggctggagtgcagtggcatgctctgatcgtagctcactgcagccttggactcttgggctcaagtgatcctcccacctcagcttcccaagtagctggaattacaggcacgtgccaccatgccctctgtggagccctttctgacttccatcaactccctgcgaccaccccgcatccttcacctctcagtccgcactccctcccaggccaactgcatggtgagtgtgtgtgtgtgctcccaggccaactgcatggtgagtgtgtgtgtgtgcaagtgtgcgtgtgaatgttaccatttttctgacttgccattcttcagtagcaactctacagatgcgacagctcttccccgttcctctttagtccatgaatggtgggcagtataaatattattttcacttgcaactcagatggattgaacaaaaacaggaataacagctcagcctcctgtatatttgacgctagatgctgttggaaaggcagctttgtggggcaaaaaaaaacaaaaattgagtgagcttgcttatatataccaaagatgctcttagacttcaatgtttctcaaagggtgagattgtgtgttacctcaccagaaccacctgggcagtgcattaaaaaggcacactcctggagcagaagctctgggacagtacttgtaggtttgattttctagccagcccccagatgattctggggcttactacagcttgaaaaccaccaagcctcagatgattagaggtaaaccaaatctggcacagatgagattcccacacagatgtattggtagatgcatttattgtatcgcctgcattggcggcgggggtctctctacacattttgtttaaacctgggccaggttaatcacgttgggaatttagtgatgtacccagcaagggtggtttcccaagctctccgggtaattgatagggcttgggttgccagtctgattgtggtaacaagcaaaggaagaagtagggactgtaaggcgggcagacctagggaacagtttccaaacagggttgggggggaaagaccggaaaggacttggtacaggaatttaaatgtttcctttcggatggagatggcaggggtcctgctgttttcagcaatagttaccgcagtgcctgtctgctgggctcctctggagtcttctctttggaccctgaaacttgagagggtctgcatgcctgaatctgaatgcggctcctttaggcccctccctttgacccctggagcctggagaaggggtgggggaagggtggggaaaacaggcctggctgttaaggtttaccaggttccttttggccacaccccccttagtataaacttaacacttctctccagggtccagtttctaaagcaaagtgacagcatcgacacgtccctaaagcaaacttgtcatgaagtccctcacctctcaaatgtgagtccctcctggcttcctttgattttatctgtaatatttagcagatacgtgtgattctattaggctagttaggaaaccaaggcccaaagaagctaggggttatttaaggtcttgtcatcagctgtaaaacaccctgctcagcgccctgctcagtgaaagacctggcatcccctcccagttgaatttcctggtaaggggcacctgagatccggtatcagtggggtactgctactctcccacccctgccctggcatctctgctccctggccccagactccgtcactggtagagctgggcagacccagcagttccaggatagcgggtggaagtgagcaggaaggaagagctgggtttgtggagaagccttccccttatggggcaggctatagggaaagggaaggcagccaccaaagctccagagcctgatggcgcatctattgggctgggacttcccagcatacaccattagggaagtgtagggttggctgggagagaacgagggagccaggggctctgggatagaggtggggagagaaggggaggtgagggtatcagcaagagggaagttgggctttcctttccagcagttgggctcccccatcctgcatcttcctccttgatttagaagaattcatctagagtgtcagtgcaaaaagccccttaccactccccctccctaacagataccctaaggctgtgcttgtgcttttcattcctgccattcatctgccaggaagctcatgcagttacagatgccccctagatctggccattcctgtctgaaaaacctgggaggttgtgcccattcctagtttccgctctttcatcttcccttgagccttcctgccttgactgatcccagctgcctctgagttccttcggcacatgtagaaagcatccctaccacataatttaccatctaattgcatgctgtcttgaatgagggctgtggtttcatgtgtggtgggtcttgggtacacaattaggctgtgagtaacagaggcctagagccccatcatgtctactagctcttgcacctctttgcttcccttccccccagcctacctactgcaggtgctaaggactgactgacctgatccggtgtttaggtgagcttcaggcaggctcccaggataaagcacaattcgcttacaggttctaaaaccagactgctcagtgcaggggttaggggggtgcccctgcccgacagcatttgcccaaatgccgctcctctccccagactcagggaggaggttctggggcagggccgaggtttggggtgatggcatagagcccaaccaggcagcacactgcagttgccatggtgacccttttgcttttcttcaggaggatggatttctcttttagcccaaatcatctgcctgggctactctgagaaccagattcctagccccctaccttccacattctaccacagggggcagcttgtcagaggctttggtttgaaatcacagtccagcatcagacagcaacaggttgtgcttgatcagggagcaggtgggaaaggcccagaggagggctcacctgggacgcatgccccgagggagctgacctccatctccttctgcttcccctcctctctccagaccacttctctccagtgaattgctctgacctttcaggtgggctcgggcctctctaggcctcagtgttctcatttgtaacagacacagccagggtctccgtgtttccacacctggctagtattagaattacccagggaggttttaatagattcctgggccccaccttaagcccgctgatctacgtaggtagaccccaagaatctgtatttttaaagccctccttgggtgatccagatgcaattaaaccaaagtgaatctggggttctcctggcgaggtgattagcaaggccctcgtgaaaagaaaggaagttctgcctgtgctaaggattgactggcctctccgggcttccaggagcttctggagagccccttacagcctagtcttgtcaggaagcgggtgtttcctggtcatgccggctaagccaagaccagacccccacttctctccctccagagaagccaggagagagagaatgacaggaaggggggatgttgccataacaacaagctccaagcagaggcctcccttccaggggctggtacttcaccccagtcctggtggtttccatggagatgggttactgagggacagggggaatgtctgccccacttagagcatcagccaggagctgccagaaggcagaacaccgctaggtgcgagcctttggacccctctgctctccgggtgcacatctgacaccttccccctgcttagttgctctctctgcgactggtctccttagcaacaagtcctgctaactccacgctgttggcttcatggctatttttagcttcactgccagcagcccagtcctggtcatttgcttgcaacctccatatggattgcaagggaataggaaaacaaagattcccagcctcttttggtagactgggacctcgtagcaacgattagaattcctgctatgaaatggatccattccccaacttggaacttgggcagtggatttgggataaggttcctgagacaacactgcattcagttcatttttaatcctctactatgggcaagatgctctagcccacactgcttccatacgaaggtgaatgaggtccagtccctgccctctaggaatatttaatcttgtaaaggagaaagatgagtacacaatcaccttcgcacaaggcaaagactggcaggtcctaacagaggcttacaaagtacaaggggagtgtggcagggatggggtagaagataagggacagaagggaaagataagggacagaagggaaagataagggacagaaggggaggataagggtgaggggccagggcagcactgtcaaaagaactttctgggatgatggcaatgttttaaaatcagtgccatcgaattcagtagcccccagccacatgtggctcttggacacttggaatgtagccagtgtgactgaggaactaaattttaatttgtatttaattcaaatagacacatgcagccagtaactgccatcctggacagtgcaaggctagaggaactttcatggtggaggtcactggcggactggtgtagaaggatatgcagggtttaggtggtgaaagttgaaggggaggaaccttccaggaagaaggaacagcatgaggaatagcaagtgggcgggcaaagcgtgggcttcatgatatcacacggaggcctccagattcgtgggggcctagaatgtcacgcagagtgttgactgattggcattgaggcttttgagctgtaggtgggaggaggacgggttcagaacagacatccatcctgacctctctttttgtctggatcaggtctccctggtcttggggttggtgggaatacgtgtggggtggaaatacgtgtgcagtgacttcatatgtgttgctttgcagatggctttatagggaccatggcttctctgattcagagtagagagactcgcatggtaaatgtcctggtgggatgacagcaacatcatctaccctcatcctgccggaactggattttatgacagagcatgccacagagcactgaactttgagtaaaacgtcaggcatgagctccgggcgttgctgctctttggagaggtatcatgatgggctagtgtctcgtctcaattctttgaggctggaaagtcacattaacatacttatgatcatgactcttcaatgacccaggagaatgatgcaccccagaatgttagatttaccaattgataacaggacatgtccttgccacacaaatcataatgcttgtcctgctgttttctggcaacttactggtatcagagtcattggaaaatgtaattaaaattggaagtttgcttgatgaatacaggatcagggcagagaggtgtccttcagaggtatttttgtcgggcttaggaggcctatgggatcactgttgtctgcccaccaaacaggaactgtgctctcattctcaggtcactctctccattggaccacactggatacttcctttccagcacctcttttaggctcttgacatccagcatattttcttctcatccaggagttcctcctttcaacatacttttataacaaatccaagaacatggcttattttacatggtgctgctatttgagtttggctatctttaactcaaacaaaagacctatctggtaaactaccagggaggtttactttggaagctcaagagcatgtgtgacctgaggatccttgaagattgccccattttagtaaggggatttttccttttactaaaatcacccctgctctagatcttggggtctcttctgctttggggtctgactgtgcattgaccacaaagaagacacatctctgtttaagcgactcagtctagaaaaggcagtgtttcctgtctgcatagttacaggagagggaagaaagatgctctgggttgttaaggggacacctttgagcctaggcttccttttgcttgttgggaagttgtgtggaagaggtaggatttgactttaatgttaaaggatgagaataaggaagtttgacggccggagggaacagctacctaatatgtggccaagagaaagtgaacagttctggccagttctgacaccttgagtccttgctgcacgcacggcatcattgcagtcttatgtcccaagaccacagaatgtggagtctaaggtgtttagccgcattataatggaagagcgggtatcgtacatgagacagtgtctgcaaaaggccaccaagccagaaccagaaacttcttttgagaggaggcctgaagtaagttctcaggcccgttgaccggcatggatgcagaagaggtatcagtgagagaagcagcgaagcaagtcatgctgcattccctaaaggttgggagcaggcaagagggacaaattctgggcttctcacttctgcttggggagcccctggaaatgtgtaccaggcaaataccttagttgggttgaaagatgggttggaacagaagagggcctgtgtttttgtttgtttgcttgtttttcgaatagcttcttttcctagctgtatggattctgggtctcagggttgggggggagtctgtacccaagtcttctaggtagaaggctctctggtttggggtccttgctcttcagggtttgcttgctctgatggccctcagggggagggggggctccctcactgcacatctcgtgacttcattataccagcacctgtatgtcccaggtgactgtggctgggagccaggcacctagagaattgtctcattgtcattaggagatggtggcgttccatggccaaagagggctgatgtcatcactcgttttgcagatgagacaacagatttcttgggggttaagtgacttgtttaaggtcatggtggtggaaacagaactgaagtccagatcttttttttttttttttttgagacggagtctcgctctgttgcccaggctggagtgcagtggcatgatctcggctcactgcaacatccgcctcctaagttgaagcgattctcttgcctcagcctcccaagtagctgggattactggcgcacgccaccacgcctggctaatttttgtatttttagtagagacaaggtttcaccatgttagtcaggccggtctcaaactcctgacctcatgatccgcctgcctcagccttccaaagtgctgggattataggcgtgagccaccgcgctcggccaagtccagatcttctaacaagtgccgctgcccaaatagccctctgctgtggggtgcattttcctccatttcctcagttcttccttctaattcatcttgccaacggcaactaggctgatttttccaaaatactcattcatcttgtcagaaaacctgcggttattcttccctgctacagaatatacccaaggacgcacctgaaggcttgccattaccttgccctgtcgtgtactgggagggtggaggtgggcgagggtctcctccctccccagcccggcagctcttgctcatcctacccatctcacctcattccaagtccgatccagcctccaggcccagtcggctcacctggaactgacctctgacctcttttgtcatccatgccgcccatttttttctacttggtatttgtggcatagttacctttacatatgtttgttttacagtgatcctttcatatttctccaagtctagtggaatcttcaacccctcgagggcagagccaacagggtctatttctttatctgatcctacagccaacgtaatggagggctgtgggtggggactgcgtctgccttgggggtaggtgcctttgttcaggaggaggaagcttgaaatggcggaggctgcacctggaggccgcacctggaggccccaggagaggagtcaggtcttctcgatctgcagatgtttgagcctgggaatgaaggaattgctgaactttctgaaggagcgccctcgccgcgaccaaccttgcaaacaggaaaatgagaaatccagggaaggcccagagtgacgcaggggccctgggactcgaagcctgacctcctcacgccgcgctttttgaggcccccccgcttctctattcacctgtagtgtggaggcgggagaccccccaaacaatccccgatctggagcgctcccaatgcctgcgcgcgcctgctgtcactctccgtctgtgtgctgagttttcctacagcttcctgggcctcctatctgtaagctttttctttttttttttttggttgtgcttcagagaaactcacttttcacaactttctcccggctctcccaggccgtccgaaagctccggcttgctttcgcccggacccccggctccctccgggcaggcggctcgggagcagccccttccctcccctcccggccccccggccccgcgctaatctcttccagagctgggggaggggccaggcggtcttcccgaaggcggggcgctccctgcagcccggcctgggcgggccctgggaacgggcggggaacggcctcgccccccggccccgcgcccctcggaccggagaagaggggctggcccagcgccagcgtcggagcgccggccccctccccgggccgctcgcagccaaccaggccctccagcggggcccacgtgacctggagtcctagacaaagaaaatgttccctccctcccccccgccgcccccctcccctcccagtggccccctccgcccccagccccatcgcccccttcccctcccccaagacgggcagctacttccagagcttcagggccgcggctcacacctgagcgcgactgcagaggggctgcacctggccttatgggtaggttcctggacggagccccggggagaccgggggcggggaggcggcagggccggtgggaggatctccgcagccctccgggtttgaaaagagtgaagtatgcctccccggcccgcagcagccgcggcgtcccggcctggcccgagttctggagatgctgacttagtgctggcccgccgccttcctgcccacgagccccaaggccccactcctcgagggctccagtggcacctggcgccctcgggggacaggaagagcagccggccgggagcccgggcagtggtaactgcaggctggggcacgcggagctggtggcccaggagttgggttggaggcagcttcagctgagctggcagatgggtgcctggcacttccctggtgacctgcgtttctggctgggggaccacgcggtcctcagggccctgccagcttgcggcctcttaagagggacagcttcctctcctgcgagttcctggaagcctccaggcaggccgccagaaagaagtggtggggggcgggagagcacggtgcgaccctgacagggggccggcctgtgacaccagggtagcccaaggccgcatgggcttgggggcggtggcgctcagacctctgacctttggaatgggaagcgctgcagtcgcagggggaggtggaggcgggacaactgcctgaggagatgggactggaccggaggcagtagtcaccggagttcccggtggctttcccagtctggacggactccaggcccaggggcttccgtgagccagcagggagtggggaatagtgtggtgtctcttgaatgccgcctggtttctccgcccccctgggggtgagcagcccgcagcccaaatgcgagagcctcgagttcacccctgtttctcttattgtttgcctggggaggaaggcaggagtgcagagggaaaggggtgctttccctacgccccagacaggcctagtctagtgaggcccctcccccacccagctccggctgccccttcccctttcgtcaggccccctgctgctcacccacccccccccccgcttcttccttcccccaccccactgccccagtcctgatggaggccccttcctcctccgccagcccttaccgggccgggtgcgtgttctgcaccaagtgggctggggatgctgaacagctcgcagcgcggcatctgacaagctgggggaagttgtgggggaagaagaatgttcaggaactcgctgtgaaggggctgagagacaaaggggccgtgggggagtgctgagggtggtgtcaggcacgactcctagacctggggagccagaggaggagcccagcccacctgctgctgccgctgccaggaactgatagcgggtcctttccccttcccgcccactgctacctgcacacctcgtactggctcaggggcctcgccacgcctggaaacggggtctttggatcatccgaggcatcaccctaggtcagccacccatgcctgaagcttgagaaggcgtggtgggcgaggagcagcggcgtggtgtgggctcctctgcgagggtagctgacgcagctgaggatcccagcaagggaggcaccctcccccatagcggggtccacacttcagtgcaccttctcgtaggggaggaggcgatggctaattgatagtaggaaggggagggcagagttctggccaggtgagagtggggctagggagcgatgagcaattactccacattcttccacccccgcagtccgatccgtgagctcattccgccatccatgagctcattttcccagctgtttgggggttgggggcaaaggatgcattatcctgttttagaatttgttgagtcagaggaagaactaggacttaacatcttcagtgtttttcggaatcgccataagaaacagattggtctgtggcttggaggcaaagacttgaagactcagtgaaattaaatgaatctgtaagacaatacattggaccactgagcttcaaacaaaggttcttaacctgggacccttggatctggggggtccattgacaggctttgggagagtcctgggaatcattggtccatatatacaccagatttttaaacaggtcagaaaagctttccagaaatccttccgttcaggtgcctttttatgttggagcaactcctagaaaggactgctccaaagtcagctgctggttgtgaggctccgacaggaatgtagaggctgcaacttcagattagagctcttcttcatactccaggttgcttgctcatcctctttacccttgaggatgggtttgcattcgcagctctaggcccatgggggagctcttttgaaaggcatgtagaaaatggggttttctcccctctaaatctggtatggtggaaatgggtgcaaagccttgaagtcagagggccgtgtttcctagattcagtaaaaggctaatggttctcagagctaagtatcaaggatttcattctcctttgtagggatcctggagcgggttgtgagaaggaatgggcgcgtggatcgtagcctgaaagacgagtgtgatacggtgaaaggatggaggctgtgcaatgggagaataactggggtttggcttattaatttgttggtgggatggcagagagcacgcctggtctatagtgcctgctcagtaaataactgaatgaatacatgtatttatggatctgaagggtgcacatagaaggggtcgaatctggtaacaatacacactgggtgtgatccataagaactccagcatggcgtctgagcatgggaacagacaggctcttggggagcttcctatggtgtttgagcatgtgctggggggagatgggctctttaaacccagagcagtgatttcttcgtcagcagacatcacaaaactagaattgtggcctggaaaccaggaataggaaggattgattgcccaggggaattgtggagaagaagagagggtcccccttgctttttaaatttttggtgacagggtcttgctatgttgcccaggctggtcttgaactcctgggctcaagtgatcctccctgctcagtaactgggattacaggcacatgcctggctgaagatcctagtcttgattaagaagataggagagggtggaaccaatatctgtgtctaaacaatagtgcctgattaatatttggatagaattaacatctccataggtagggggagggatagcattaggagatatacggtataatgctaaatgacaagttaatgggtgcagcacaccaacatggcacatgaatacatatgtaacaaacctgcacgttgtgcacatgtaccctaaaacttaaagtataataataataaaattaaaaaaaaaacattaacgtaggctagacacagtggctcatgcctgtaatcccagcactttgggaggctgaggcgggcaaatcacctgaggtcaggagttagagaccagcctggtcaatatggcgaaaccccatctctactaaaaatacaaaaattagccaggtgtggtggtacatgcctgtaatcccagctactcgggaggctgagacaggcgaatcgcttgaacctgggaggtggaggttgcagtgagccgagattgcgccactgcactccagcctgggtgaaagagtgactctgtctccaaaaaaaaaaataaataaataacatttccgtatcatagggtaggcaaccgatatgctgatgagattgaggaagattagatctgggaggaaggtttcctaaaaggcagaaaagaaggctggaattgtgtgacttgagttcacctctctacttcaagtctttccatttttcacggcaaggcagctggttggctctctccagcccttgaccttctgtcactgttccgggttttgacaaacatggtccccttgagtgtcaggtggccttgctaattcctggagaggtcaaggtgactttttgttttttgtttccccaggctgagaagaaagccaaggtcattgcaggaatgaatgctgtggaagaaaaccaggggcccggggagtctcagaaggtggaggaggccagccctcctgctgtgcagcagcccactgaccccgcatcccccactgtggctaccacgcctgagcccgtggggtccgatgctggggacaagaatgccaccaaagcaggcgatgacgagccagagtacgaggtgagcggcttcttcaccagccgtggccagctctttccgggggccttccactgccagctcctcccgtcctgctctcctctctccatctcccacgggccgctcgctctctccacctgaccccgggcctcttcgtctccccgattttgctctgtcttgcctcattcagatggagctccctcctggctgtcctgccccccagatgcccagggctggggttgccatggggggatcagggtggcagggcctcgtgaccactgtgtaatgatttctgctccttggggctccaggacggccggggctttggcattggggagctggtgtgggggaaactgcggggcttctcctggtggccaggccgcattgtgtcttggtggatgacgggccggagccgagcagctgaaggcacccgctgggtcatgtggttcggagacggcaaattctcagtggtaagttgtggggtttggcagtagcctggggtgggggaaggttcttgatcccagggcctgtggttttggctgggggcagtctggaattggggtgatggaagtgggttctgctcacccctactctgcctgtggtgggtggaagaagctgtagggataattaggttgcacctgtggctcagagagctaggatgctggcccagttttagccttccaggaacctattcactagaattcctttccacctgcactcaggtacttccctactcttggtctggactcctctttgcatcgggtaattatttatcactgtatctggtcccctccagggctgagactgactctcgaggctcccagcccctgccagttgcaaggcatggggtgggtgcttgcaagtgtaagcctcggcaaacaaggcctggcttgtccccccaggtgtgtgttgagaagctgatgccgctgagctcgttttgcagtgcgttccaccaggccacgtacaacaagcagcccatgtaccgcaaagccatctacgaggtcctgcaggtgagtgtccctgctgggagctcggaggaggagcctagagcactaggaggcctggaagttggagtgcaggtcgggcttgagcagaacggaggcccaggaggcagcgactcctttctctccacctcgctcgcccgtatgcacatcccgctcctcctcagcggttcctccccatcttgcctgtgccaccctcactactcagagtctggccttgagcctgacccatctgccttccagcctgtcctgacaaccccaaccctggcgtgtcaccctccaggtggccagcagccgcgcggggaagctgttcccggtgtgccacgacagcgatgagagtgacactgccaaggccgtggaggtgcagaacaagcccatgattgaatgggccctggggggcttccagccttctggccctaagggcctggagccaccagaaggtaaatgagggcacccagctttctgggacccctgcccgccaggcagatccacaccagggctgggaaagccatgcttagggagggctgccaaggcctccacagaggggcccagtgggaacagccggggtctctcttgccctggggtcaggacttgaatgacctgctctctgttggttggctagaaccgtagagttgggaggtgggaacaagttggagaccaggccgcaccccactgtaaggagggtgggggaaggggctggagtttcctgtcagcctgtaactgaccttggcacctgctttcctcctccagaagagaagaatccctacaaagaagtgtacacggacatgtgggtggaacctgaggcagctgcctacgcaccacctccaccagccaaaaagccccggaagagcacagcggagaagcccaaggtcaaggagattattgatgagcgcacaagaggtagttggcctgcttctggagagggtggcaccaggaggcctgcatccgaggagcaggcgcggcctcctctgacgccagctctcctccccttgcagagcggctggtgtacgaggtgcggcagaagtgccggaacattgagggtaagtttgttgcctgggaactctggcactcctagtgtggcatggggcagagtagcagaggcacttaggaaggtttgaatgacatgggaccaggggccacgtcctgcgtgtcacaccaccttcaccgtgcttcgtgcactgtctgtctttctggtgccatgattctgctctcctgtcaatggcgtgggctggtcttcagggtgtgggactccccagtggccacagaagggcagcccctcttgggtgtgactgaactcacctacaggtcccctttcagcctttcacctttgctcacctctctcatcctggggctgtcgccctcagcctggtgctggtggatctggctcaggaccctcggcctgggaggattggggcaggggtcaggacttgaggattaccccgggaatccctcctgagtaacctggaaccagggctgcttctcatgagcaggtttctgtcctgctgtcactccatccctgtttgtggatggtggcattggtgctctgagatgatggcgttcgagactggggtcacagtgcctcccttttccgtggggcaccctggactcttttctggctgccgctctggcacaccccttttttgtggctggtctggtgctgggctccggcccggggagggagaggcccttcggtggtactcaccccatcccctccctctgctttccagacatctgcatctcctgtgggagcctcaatgttaccctggaacaccccctcttcgttggaggaatgtgccaaaactgcaaggtaggagcacacccacccaggagaggtgccgttgaggccggcccttcggccttctggctgactgtgtccaccgcttctgggcatgtacagggtgacacccggtgtgcgtgtgatgtagacttcagagcttcctctggaagcagagtcctgatgacacgggcccttgggtttcaagcttttgacttgctctcatttttctcctctgaaagattctttacctctctcaagccccattttagtccccagatgtggggatgggagcagagcagcggggacaagctcaggcagagtgtagcagggttgccgtgccctggggagcccaggagaaaaacccagggctgcaggaaggcccaaggtggtggtgcagccccctccctggccttgttctcaggctgctctttggttctgtccatggcggtgcacaggcagatgaggtttccccttcccagatctgcagctggggcctgaccctggctaaggtggtggcggggtcatgtcttcagggcttaggctctgtgaggccaggtgtggagcctcccttcgtcctggccgtcctgggacccgtcctggtggtttctgacccttcccgctgctgtctagaactgctttctggagtgtgcgtaccagtacgacgacgacggctaccagtcctactgcaccatctgctgtgggggccgtgaggtgctcatgtgcggaaacaacaactgctgcaggtgaggctgttgtggcctccagtggtctccttagctgggccccaggctcccgccgcccaccgcctcccctcctctcccttccccacagaccctccaccccctagccatgctccagacccggtctttccattccaggtagcacaccttggcctccccggaccagggctgagagtctcctctgctcactgggtctccttccaggtgcttttgcgtggagtgtgtggacctcttggtggggccgggggctgcccaggcagccattaaggaagacccctggaactgctacatgtgcgggcacaagggtacctacgggctgctgcggcggcgagaggactggccctcccggctccagatgttcttcgctaataaccacgaccaggaatttgtgagtgctgggcctggggcgcggtctcgagctcccttggctgagccttggttgtggggcctgagctgtgcgcagggtgtgtgggtctaggagcctggctggagggccagcgctgggtgggagcttgggacaccgctgggcctgcatctgacctgttgtgctcactgcttaggaccctccaaaggtttacccacctgtcccagctgagaagaggaagcccatccgggtgctgtctctctttgatggaatcgctacaggtgaggggtgcaggcccaagaggtgctggccgccttgtcccaggatgggggagccaggtgtgtgggcaactctggcaaaacgaaatgatggttagcttcacagctgttaaaatattatcccaaaggggaagcttgtgtgtgaagtggaaacgtatgcagtccgtggacttagatttgaccaaaaataaaaaagtaaaacaggtggtaaagcagcctgcctgctgggcgcttccttagtaaggaagattcagccacagaggccgagtgatcattagtcagattcacagagatttcacagagcctcaaaggacagattcagcaagacagaaacatacacagttctgttgtgttttgattatacacaagatagtctaaagaagacttatttaaatcacccattatccttttattaatattttggcatatgtccttttcagatttttttctgtgcgtttgtgtttatataaccatacatccatagatagataaattaagtgggtagtgaatgcacacggttttgtaacttgccttgcatttattatattatgattgctttttcatgtcaagcgatataaagctacatttttttttttttttttgagacggagtctgttgcccaggctggagtgcagtggtgccatcttggcccactgcaacctctgcctcctgggttcaagcagttctcctgcctcagcctcccgagtagctgggactgcaggcacccaccaccacgcccagctaatttttgtgtttttagtagagacggtgtttcaccgtgttggccaggctggtctcgatctcttgaccttgtgatccgcccacctcggcctcccaaagtgctgggattacaggcatgagccaccagacccggccaaagctacattctttatttgtaagtgttgcgtaaggttccattgagtcaaagtgccgtaatttatttaacgaacccctattaggtcatttactattttttttattattattataagtagtgctgtgacaagcatcctgatagaatatatacttgaaccattccctccttaggaaaacctttaggagatgctgggtcaccctgcaggcattgtttgcttcatgtgactctggacactgcagaaagcgtcctaggagattgtgaagacacatccacccctctgccctgcccccccagccaagggcttcccggcttttaagaaaggaatagctcttcttagaattcatgtctttggccagagggagataggcagtctcaacccagggaccctggttagagggtgaggcggaggggatggcttccccggccagatgtttgtcttctgttctcagctccatgctgctcttctgtaaacctagagaagttcatttacagctttttgtatcccagactggaaccttgaggccttcttaaatcatgtcacaccccaggaggctgtaaaagctccttggctgggggacatgttgggagcagcagaaacagtgccgtaccctgtttctcccgagaccctctcgggatctgctctgattgtttccttgcctcagttcggctagcatttctggagggggagggatcgtggtaaggaaatctgtgtggttagctcgggggaagcacaatgaaggaggcctcctctgtctggaagagcctttgcaatccacgagagggctgagtgaggaatggaaactgctctgatggggaggcaagggatgcacagagctggggactcatagcggagggggcggtgattccaggggctactgggtggggcagggcatttatgaaacctttgtggagcagggaacattcaggttgagtcaaagggtggggtagaattgtagcaggaggtgataagagggtatggaaggaccaagttgtagacggacaacaacaaagaaaaggcccccaaaaagtcagctgtgattcttcctggatcctggaaccatgtactctccggtcagttttctgcagctttgactgccttccagataggccaaacgaggcatgagacagagcctcgatgtccttactatggatactccagttggatccagaaaggatttaaggcatcttcagggaaaagataggacttgggcctacagctgaccccatgggtcctgttggccagcaggctcacctgccgagaccagggtgccagggagatggctccaagtaacggtgctgtctgctggctggtgcagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatgtatgtccatgctgtggggcgcagcccgtcttcccctccctgcacactcagcacctggaggcagcccttgcctcctttcattttgttcatttgccctctttctcttcaccctttgccccagcctctttttctccttatcttccatttgtcctctgtcttgagagggtaaagggcaaattgaaatgggaatttgggctaggcatggtggctcatgcctataatgccagcactttgggaggccaaggtaggagaatcccttgaggccaggggtttaagactagcttgggcaacatagtgcctacttgagctctggctactccctgggctggtttctggacagtttctagtagagctctttggattagagagaggttcctgtcctcccaatacttttggcaaatagctttatcagcttccatatctgggaggctcaggcagggtcttcccttaagcatgactacaggccctctggagtcctgaagggcaactggaggacattcacatggcccagacaccctgcctaagacactcagcatgccaggtaccttcagactcagccgtccacttgggtcaccttcccagtgggtggctgtgttgcctgcctgggtgatactagtttccccattggcctaagaaatgatccaacttggtcccgttcttgtttaggacttgtccatggttgcagctagggtcgtgagcctgcagagcagctgggtctcctctctttcgtgtcaaaggacttctttgccaagttcacagataatttctctttcttcctgtctgcctctgtccctggacagcaggcccatcacgttgcctttatcctcccagatccaggagtggggcccattcgatctggtgattgggggcagtccctgcaatgacctctccatcgtcaaccctgctcgcaagggcctctacggtaggtaccatcctgtcccctccaccttctcagctggtgcttccgcacataggcttcctccttggatatttctgccctgggacagctattcccgatgaccctgtcttcccgtgccctccccgtgcccgagccacaccactgtcctatgcagacagccccagctgatggctttctcttccgacctctcagagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgaggtatagccagcaaccttggtttggccagctcactaatggcttctaccttggactgctgcttcatcctgtctcatctgcattgtggagctggggactggtgacttctgctttgcaaggggcctgtttgggaagccccaggcttcctggatagaaagggaggaagcagggttagaggcctacctcggaaagaaccagtggtctcactctaagaggtggcattgtgggtgtggacctccggggttgaaggacctgtcactcaatcaggtggggtgccagactcctggggttgggtattctgtatggtgtttgttacacaccaggatctttgagaggacaaaatcagttttccctgagcaagaagggcctatcctcactccctggtaccatgacaggcacctgagaagaaaacaagacttagctcagttttcattccttatcatgtgagtccccgtttaaccctgagccaagtctgagccgcgtgcagagggcttctgctgccagaggcgtgcttggagtggcccttctgggagcttgagcccacctgtgctcctgagtcttggggtgggtcacactgtgagctggtgttctgactgtaagtgtgagcaggcgtctgtttgagttctaatcagggctcatggtaaacacacccatactcgctggagcctgggctctcccagcctctgggtagtctagagcagcactgtgcaatatggtagccaccagccacatgcggctgtttacattaacattaaccattcagctcctcaatcacattggcctcattttaggtacaaaatagccacctgtactagtgggctagtggccactgaattgaacagtgtagaggacaacccaccatcgcagaagttcatttggacaatgctgatctagaacgttccagtgacagcttgtggaatgtggctaggtgtaattccagcttctcctggaggctgcaggctagcccagtgtgtggctcctgagagagaagtaagaggctttgaggctttaaggctcgaccccagcagcatgccggcgctgtttcatgctcctccttggctcatcttcaaaccgtctcctgttttgtagtccaaccctgtgatgattgatgccaaagaagtgtcagctgcacacagggcccgctacttctggggtaaccttcccggtatgaacaggttggtgaaagctcctgggcctggggggctgtgggctgggactgcaggtgggatgacccatagtggggaagcccctgagccgggccctctctggccggcctctgctgaatgactagctgctctgccatgttccttatttccttttccttgaggaggaagaggagtcagggtatagaaaatactcgtctgaaaccaacaactagtggtcttaaagcaggacctttaaaagtactctctttataccaaagaagagtcctgtgctgctgagttaggtagttggttttctgacattggtgatgtgtcccgagccctctggttcttagattctcctgaggcaaagccatggggcatggctttggggctgggaaggggagttgtggccacagcctgttccaggagagggaatcttgcagcggctgcatgcaagttcagggttctgcttaacttaattttactaatcacacactttgttggcttcaggtccttttttggagagattttcttcagttaggttacacagataaagattttaatatctaggaatgaagaaaattcatttcatcagttataaaactggcattataaaaaggctttttaggcgaaataaattgtccagagttggcaattcaaaagcatgctgagagccgctcagtccctgtccattgtggagggaagcgttttccagagtgttctgagtcatgggtgttaaacctccatggtgggctgcttggtatacttggtataactaggagacatcatttcacctggcctcggtcaaataatctccctccttgctattcccatgaggttgtaggaccttccccatctcatacatgtttggtagagttgctatcgagggcttgttgaggggaaggagatggggcattggggcagctggcctgaaactgtcccccgagagttcccaagatacatgtgcatattgggaaatggatgggctaatgaggtccagtattttcatggaaccactcagtgtctcccgacagaccttggtcctgtgggatttctcgcctgcttctttcaactctaaaatactaaactctgagcttctaggaggagcagaggtcctgcatgcagggccgactgggcagtgcacgcataaagagtcggggacatctgtcgagcacctgatttgactgcaggccccgtggttcccacactgagatcctggagccagtgctcagggaagccttccttaaatatccttacatgtcagaaaaggtcagcgttgaaggggacagccagtcagttatgtggcctgcaccctgagaagtgacatatttaatgtcacagaatttttggtctttagggaactgcagaatttaagtctggttttttttttttttggagacagagtcttgctctgtcgcccaggctggagtgcagtggtgccatcctggctcactgcagcctccgcctcccaggttcaagcgattctcctgcctcagcctcccgagtagctgggattacaggcacctgccaccacgtctggctaatttttatattagtagagaccgggtttcaccatgttgatcaggatggactcaaactcctgacctcaggtgacccgcctgcctcagccttccaaagggctgggattgcaggcatgagccactgcgcctggcccagaatttagtcttttaaagcagtacatttaaggcttgcggtttgattaatttcatccagtggacacacccgtgtaacaatcaccacaaatcaagatacagaacattgtcacccccaaagcttgcttcgcagtgggacgcttaaagctcagtgtattttagactaactgatgtaaagatagctgtgctttttttttctttttctgctaggtgttatctgcctgttcacaatttgagaccacttttgaatttacctttaaggattaacgcttggtggatttgtgtctttgcatggctgcagtgtgtgttcgtgtgtttgcggggagtttgaagagagttatgacgtgtgtgcgtgatttggggaaggggctcttaccctgtgaactagtggctgctgggggagggaggggagtcgtgcagctggggcccttcccgctgttatccaggtttctgttgttacagtctctcttctgcctcctaggccgttggcatccactgtgaatgataagctggagctgcaggagtgtctggagcatggcaggatagccaaggtcagctccagcgtctagaacctctgctggggggcagggagaaggaagggcaggatgaggtggagcggtggccagggccttgtcccgcctgcttctccagctagtgtggagaatgcaatttaggagacggtgcagggtcatgcagcagggcagccagaggagggcccagggctgctggccttcagcccttcctccctggtgagtgtcgcccatcacccccggtagacactgaggagtctttaccaagacctggttgtgctgtggcttttttttcatttttaaattttttggccaaaacctattacaaaagccctcaaagcagcctcctgtgatgtggaagctccatttctctctggagctggaatctcccggagtgactacctggggcactgggttctgtgtcagcaggttccagcattcagaaccactggcagctccgagggcatctttgagcccctcagtaacttggcatcctttagcctacaagttactccatctctgaaagaactaattctttggaagacttggaaagtacagtggctacagcttctacttctaaagtctgtggctaggccgggtgcggtggctcacgcctataatcccaatactttgggaggccgaggcaggcggatcacctgaggtcaggagtttgagaccagcctggccgacacggtgaaaccctgtctgactaaaaatataaaaattagccaggcatggtggcgggcgcctgtagtcccagctactcaggagtctgaggcattagaatcgcttgaacccgggaggtggaggttacagtgagctgagatcgcaccactgcagtccagcctgggtgacagagcgagactccgtctcaaaaaaaaaaaaaaaagtctgtggctagatggatctaagattggccaggtgacctccaagtaggaagaaaagtgaggctgtggggtctgtggaggctcctgccctgcgaactctgctcactcagagccatacccagccccctgggtggaccaggccctgtcagggtttggcgagtacctggcatatttggtagacgcatgaccagtgttggctggtgaatgaattcgtgcttattcctcttttctcctcttcatctagttcagcaaagtgaggaccattactacgaggtcaaactccataaagcagggcaaagaccagcattttcctgtcttcatgaatgagaaagaggacatcttatggtgcactgaaatggaaaggtacccgggttgtgctggcatctggctgcggtgtggctgttctgattgctgtgcttgctgcgggggtcctctgcctttcccacctgacttgttttccacaaacgtcctgggaacagtgggttttatcaagcatttcccaacatggcagactcctggggtggactcctgacaccccaggctcctgggtattttaaggctggtcccaagtaagaggagtctaggggccaggagcagtggctcatgcctataatcccagcactttgggaggccgaggcgggtggatcacctgaggtcaggagttcgagaccagcctggccaacatggcgaaaccccgtctctactaaaaatacaaaaattagctgggtgtggtggcgggtgcctgtagtcccagctactcaggaggctgagacaggagaatcgcttgaacctgggaggtggaggttgcagtgagccgagattgcgccactgtgctccagcctgtgcaacagagagattccatctcaaagggaaaaaaaaaaaaaagaggagtctaggaaatcccctgctgccttgtctcccagagaccctgctgatcccccagggaaccactgcagtgacaggaccttaacttgtggtgccaattttataggtcttttgtaaaccaagccagcatagggccaggcagacctcgcagcctcaacaccctgatttgatgcaaatgggactttcggggctggatgaccttcttgttctctgagtgtaactaggatttagccagacaaagagaccatttacacaaacaaccagagcaaacaggaaatccttcagcttctgaagtcaggcaggagttcggctcctttttcgccttccctgctcttgatggggaggatgctagggctacgtgttcgttacatgcagactccaggtgtacgttgccgagctaaccctgcaggagaggagaactagagagtgtgtcctggaggtatttctgagggattgttcaaaattactcttatttctgctgggttgtgaaactctaggcagtgatgaccttactacctttaaggtcacagaaaccagcacagtgcctggcacatggttggtgatctgagtgccgggttgtttataaaggacagaagattcggcagaactaagcaggcgtcagaggagttggtgggtgtgagtgcccctgtccctgcacttcgggtggctgctggtcctccgggtcctgctgtgtggttagacggcttccgggcagcctggtctggccagcactcaccctgccctctctgccttttctcccccagggtatttggtttcccagtccactatactgacgtctccaacatgagccgcttggcgaggcagagactgctgggccggtcatggagcgtgccagtcatccgccacctcttcgctccgctgaaggagtattttgcgtgtgtgtaagggacatgggggcaaactgaggtagcgacacaaagttaaacaaacaaacaaaaaacacaaaacataataaaacaccaagaacatgaggatggagagaagtatcagcacccagaagagaaaaaggaatttaaaacaaaaaccacagaggcggaaataccggagggctttgccttgcgaaaagggttggacatcatctcctgatttttcaatgttattcttcagtcctatttaaaaacaaaaccaagctcccttcccttcctcccccttcccttttttttcggtcagaccttttattttctactcttttcagaggggttttctgtttgtttgggttttgtttcttgctgtgactgaaacaagaaggttattgcagcaaaaatcagtaacaaaaaatagtaacaataccttgcagaggaaaggtgggagagaggaaaaaaggaaattctatagaaatctatatattgggttgtttttttttttgttttttgttttttttttttgggtttttttttttactatatatcttttttttgttgtctctagcctgatcagataggagcacaagcaggggacggaaagagagagacactcaggcggcagcattccctcccagccactgagctgtcgtgccagcaccattcctggtcacgcaaaacagaacccagttagcagcagggagacgagaacaccacacaagacatttttctacagtatttcaggtgcctaccacacaggaaaccttgaagaaaatcagtttctagaagccgctgttacctcttgtttacagtttatatatatatgatagatatgagatatatatataaaaggtactgttaactactgtacaacccgacttcataatggtgctttcaaacagcgagatgagtaaaaacatcagcttccacgttgccttctgcgcaaagggtttcaccaaggatggagaaagggagacagcttgcagatggcgcgttctcacggtgggctcttccccttggtttgtaacgaagtgaaggaggagaacttgggagccaggttctccctgccaaaaagggggctagatgaggtggtcgggcccgtggacagctgagagtgggattcatccagactcatgcaataaccctttgattgttttctaaaaggagactccctcggcaagatggcagagggtacggagtcttcaggcccagtttctcactttagccaattcgagggctccttgtggtgggatcagaactaatccagagtgtgggaaagtgacagtcaaaaccccacctggagcaaataaaaaaacatacaaaacgtactggtgctttcctgtctaagtcgccttttgtgtgttcttttatgaggcccccaccatccacccctctgtgcacaaggcggctcccggcccctggaatgtgatgtttttggtcatctccaaaggctgcagttttatactgggaggctgatgacaccttttattataattattcttatggttctggttataattatgtttgttttaagattttctttaagaaaacaaaaacccaacacccttccctttaggtttcaaaccaaggtgcgggagcagggtgcttctgagttagcggtggctctggtgccctggctcccacccctcgggccaggtgggccactgggcatggtgacagctgggcggccagggagtccccaaggtctgccttctgggcttgtcactcttctcttctacttcccctcctcttgagtcgggtgtgtcagggctggagggaggaccggccgtctctctccttgtgtgtgtgattggagtggtgtgtatttcttttctagtacttggtctgatgatagctgtgctcttacctcgagtcagcagcacctggagccccaagtgctcaacactttgttctacttcctacaaggcaggcggcctggcgtcagtagtaggaagggatggcattgttggctccagggtgagggcccgacgggcaggtgcttgcctgggtagatggggtgtagatatgtggcatatagagatatatattttataatgggaggggggatggcacctcctgggactggtagagctgggaggtgtgtcggcaagcacatggccccaagtccacgtccctagaaagatggggcctacagtgataggcgctctataaatacatagatagggtcatgtttaagaaatattatggggtggggagtgggggaagcaggcttagtgtgctgagcctcggtccccactgcagacctaaaccccacaccaatcagtcaatctcacacgcatacacaggagcatatttctgtactgatgcataatgaatgcactcacacacacacacacacgccagggggctcccagcactcttctcccagggttcaggactttgtggagtcagcccgtgcagctatttacctctgttgccctgattgcaggtagactgtcaatcccagaagttgctaggtgctgaggcaggaaaaggaggaggtttcttttagcaggtgctctcctctagcaggtagtgttctatctcttttgcccctggtgatgtcaaaagattcttctgttagcaacctgtgatactgacctgcaactctaggcaccagtcaagtcagccctcaactccccctacctattctccccaactgaaatcaaattgacatccaggtctgtcttggccagggggcttccattggggaaaaaaaatgggacagtgaacctcacctattgaataaaccaaattagtaattagtgacaaaaccaaaccaaggctttggaccccctgctcattactcaggagtaaattcacacagctggtggccggtctcacccctttttggggtgcttggtcctggaccccaatctactgtctcctcgacagattggtgtttaagatcatgcccagttgcttcctgtcacctcttgacaggtggtgactgggctgctgtggggctggagcagcgaggaagggagcctctgccccctcagtggagtccctgtgctaactgtggtgctcacccagctcctccttgctcccgcggggctctgtccttcgggcctggagtccaccagcgccactccaggagggctgggggaggaggggctgcctcaggcagtccaaggtgctccctcccccgctggccaatgctaaggtcttcccggctcccagggtcaggacagagtcttctaaggctcaggctctcccaagccactgcttagtgacagttagcctttcccagcggcccaagctccaccggaactgggcctggtgctccaaccaaagggttggtgttgggacttgggaagagacttggtagccaagggcatggcaagcctgggggattcctgtgggccggcttgctcgaggagggagtgctgggcacagaggagcccttatggctgacagcttgagagcacttgttcctccagatctagccacttgaccaggccctagaatggaccagatgccccaggggtcatggcccccccccccccccccttgcagggacgagagttaacaatcaccagcttttcttccggcccagcctgtgtgcttggtctcacagtagaatttaggagagtgtggggctttgggattgtgccaacttggccaagctgagatatgcttaatccacctcaacgctggggcctctggggacaggtattgggtcaccttccaaggggtgctgaacttcagcaggctgtagataaggcagaggttagaggcagtggagtgggggaaccaagaaggcctagggaaaattgccaggcatattccctgtggaattttctttccgaagagtaggcccatttcccccttagaaacaagggttctgggggctgatgacagcttcccctgcaggtgccaggtggcgcatgatgccaaagagatccgtggctcttgcctgggggaggtagccaggtgctcgtgctggggacacagtccttggcctcctgcctaagcaggaagctagtttccagcaggcggctggtactaaccccccgccccacgggagcgacaggctggtgcaccggcagactggggggccgcaggcgggcactagcaggggtctgcagtccatagatgatgacatgaatctgttgctgtccttttgtctctaggccccagagacttcttgctattgaacatcctattggaaagcaaactttccaagagagcacccgggacctgttagcttttgaaacaggcctgggtgtgattgactggtagagatgcctgtgtgatttttggcgcattgcaggagtgtggattgggacaggcaccccagcaggaaggaaggctgacaccaaaggggtctcacagggcccagctccccttgcttcccgataattggccaccaggccatggcgactctggggaagcctagatgggaacacccaccttctccagcagtagaaggaatatttcagcagagccatctgggagatgttcttataggaaaactgcagtcattcatttcatagttttgcttatataaatagagtacatttgggaggcagttttgaattcctgttgctactctgcttggtgcggagttgcagtggccccaagtgtctttcagagctgttgcttttaggatccccctcaagctgtgggttagggccagggcagggagggggtgctgcctggcactgtgccctgaagagggctgcttgcttcctttggtcgggagcacggcagagccaacattctggtgggaaccgagtttctagccaggcccctgttttggtcccgggaggtgagtaatctgagtttgtcattgcgtattctccacgctgcctgctctcagtgctggagactagagagcgaccctggagttgtcctctgggagtcctgcctgtttgccagggagcagctgaaacctccccgctcccaatcccgggggtggaaaaggtgtagtggaggaatggtctggagctgggatggcccttctggccatgccctgctgagccaggattaaggaaagtgtcagccgcctccttaccaggccctgtgctggggtcaggaggaccgggaggagagggaaggggtgagaggatggactctgctgttgtagtgtgagctctggaatctttacgttttggtaaaggcaggccagggtctttgaggatcgagttggggaaccttgcctctgtgtttccgagacccagctaaggtgggcctttggtgggtgacctgtctctgacccgaggcagcagagttttgggggagggagaaacaagcaaggagcagggaggacaaggacacaagatccctttgctggtggtgacttactcacttcccccaccagagggcctgggactcccgaaactcccgtaactggaaagctccctggctcagctccactggggggtgaggagggcggctccctcagccccagcctagcggcctccaaccctgtgcctgagaacatgagcagcggtctaaagcatcccctagtcccatttcacttcacagaccgatttggttgcacagccctagaattcaacagacaacaaaaatgctgaaattccttagaatacgcagagggaggaggtgactcaccaaggtgctaaaacattttattaaaaatatatattgttaaattaagtctgctgtctggacaatgactgtttgttttgttttcttgtagtggagtttaaaagagactattattttactctgatatattattattaaaaaggcattttaactttgtacttgaaaactaaatgagcgatttcatgtgttttagctgaggcattgaagtagcgggtgcttacttatttgtgggaaatcatgtattcatactgaagaataaactccgtagctgatttggtaataacttttactgttacagacgatttcgctttgaccccggtggcagagcacttttactccacattccacgcagctaaatgcaggacttcccagactcttccctccccagccctctttcctttctcttaagttcctcttttctcctttattattattatttttttttcctggttccagcatcctttcaccactgtattttttttttttttttttttttttttttttttttttttttttttttttttttagggttgcttctctatttattgacaataataatgtacatttcacagtctggttctaggacattcagggaagggcgagtgggggcccgaggggtccctgccccacccgcctggtgtcctgttgtatctctgtgtaagtgatgctcgtgacatagctgtttatgaaataattaaagaggtcaatgcgtacaacagatgtagaaagtctgtcagttccgccttcatcacatttttttttgtgcccagaagaatataataaagctcctttctaatgtacttgtgctggagaacacttgaataaatggactgtttttgtgcaaaaagaaaaa

SEQ ID NO: 28 - RNA, 1714 nt, Homo sapiens
  source 1..1714: mol_type = mRNA
gcgcgggcggcggcggcggcgagagcagaggacgagccgggacgcggcgccgcggcaccagggcgcgcagccgggccggcccgaccccaccggccatacggtggagccatcgaagcccccacccacaggctgacagaggcaccgttcaccagagggctcaacaccgggatctatgtttaagttttaactctcgcctccaaagaccacgataattccttccccaaagcccagcagccccccagccccgcgcagccccagcctgcctcccggcgcccagatgcccgccatgccctccagcggccccggggacaccagcagctctgctgcggagcgggaggaggaccgaaaggacggagaggagcaggaggagccgcgtggcaaggaggagcgccaagagcccagcaccacggcacggaaggtggggcggcctgggaggaagcgcaagcaccccccggtggaaagcggtgacacgccaaaggaccctgcggtgatctccaagtccccatccatggcccaggactcaggcgcctcagagctattacccaatggggacttggagaagcggagtgagccccagccagaggaggggagccctgctggggggcagaagggcggggccccagcagagggagagggtgcagctgagaccctgcctgaagcctcaagagcagtggaaaatggctgctgcacccccaaggagggccgaggagcccctgcagaagcgggtgagtcctcagcaccaggggcagcctcttctgggcccaccagcataccctgagagtcagggacttggctctccagcaggtcccaggaaggatggtctgggtcgtggctaaaggtctgcttgccaaggctatggcctggaggctactggctggatgcagcctgcgcatatgttttatttggcccatagagtgttttaaacatttaaaaaattagttgccagtatttaaaaatcaaaaaatttcacataaaaatctggagttttggcttctcatgaaaaaaaaaaaagctagatctggcaacagcgggctttcataacgccaacgattgctagactgggataatggcggtccctccatcgccttctgtggctggttgtgggccttagttttctgcagctctacctggcctgcttactctcccacgtgccatgcagttcctgggggttgctgtatttgtagcccctggcctgggcactcaagggcagcagataccctgtttgcctccctgagtgcagaggtcctgagcccaccctagttgggctgactcaactggaaatttggttgtgacagtggcgtggggagagggctgggtgattgtattctgtgtactgcccagcccaggcctcttcatctggggactttttggcctaaccctggaagcctggaaagttgcccacttttctctttcaggttaagccagcaatttcagggccaaccgagctgtaaacatgttagtaatgaggacaactagcatttgtacagggcttcacagtttacaaagcgctttctcatacattatcacatttgatcctcccagggccctgccaggttgttttgcatatgtgcattttaatttcaaaaagtcttccttccaagcgtgtatgatgaaatgagtaaattgattaattggcgtaacttattttgcatggatccaacctaatgttcatgcaggatagagaacatttccagaatacaaatttccaaactta

SEQ ID NO: 29 - RNA, 3638 nt, Homo sapiens
  source 1..3638: mol_type = mRNA
gatctccgcagccctccgggtttgaaaagagtgaagtatgcctccccggcccgcagcagccgcggcgtcccggcctggcccgagttctggagatgctgacttagtgctggcccgccgccttcctgcccacgagccccaaggccccactcctcgagggctccagtggcacctggcgccctcgggggacaggaagagcagccggccgggagcccgggcagtggctgagaagaaagccaaggtcattgcaggaatgaatgctgtggaagaaaaccaggggcccggggagtctcagaaggtggaggaggccagccctcctgctgtgcagcagcccactgaccccgcatcccccactgtggctaccacgcctgagcccgtggggtccgatgctggggacaagaatgccaccaaagcaggcgatgacgagccagagtacgaggacggccggggctttggcattggggagctggtgtgggggaaactgcggggcttctcctggtggccaggccgcattgtgtcttggtggatgacgggccggagccgagcagctgaaggcacccgctgggtcatgtggttcggagacggcaaattctcagtggtgtgtgttgagaagctgatgccgctgagctcgttttgcagtgcgttccaccaggccacgtacaacaagcagcccatgtaccgcaaagccatctacgaggtcctgcaggtggccagcagccgcgcggggaagctgttcccggtgtgccacgacagcgatgagagtgacactgccaaggccgtggaggtgcagaacaagcccatgattgaatgggccctggggggcttccagccttctggccctaagggcctggagccaccagaagaagagaagaatccctacaaagaagtgtacacggacatgtgggtggaacctgaggcagctgcctacgcaccacctccaccagccaaaaagccccggaagagcacagcggagaagcccaaggtcaaggagattattgatgagcgcacaagagagcggctggtgtacgaggtgcggcagaagtgccggaacattgaggacatctgcatctcctgtgggagcctcaatgttaccctggaacaccccctcttcgttggaggaatgtgccaaaactgcaagaactgctttctggagtgtgcgtaccagtacgacgacgacggctaccagtcctactgcaccatctgctgtgggggccgtgaggtgctcatgtgcggaaacaacaactgctgcaggtgcttttgcgtggagtgtgtggacctcttggtggggccgggggctgcccaggcagccattaaggaagacccctggaactgctacatgtgcgggcacaagggtacctacgggctgctgcggcggcgagaggactggccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtcttcatgaatgagaaagaggacatcttatggtgcactgaaatggaaagggtatttggtttcccagtccactatactgacgtctccaacatgagccgcttggcgaggcagagactgctgggccggtcatggagcgtgccagtcatccgccacctcttcgctccgctgaaggagtattttgcgtgtgtgtaagggacatgggggcaaactgaggtagcgacacaaagttaaacaaacaaacaaaaaacacaaaacataataaaacaccaagaacatgaggatggagagaagtatcagcacccagaagagaaaaaggaatttaaaacaaaaaccacagaggcggaaataccggagggctttgccttgcgaaaagggttggacatcatctcctgatttttcaatgttattcttcagtcctatttaaaaacaaaaccaagctcccttcccttcctcccccttcccttttttttcggtcagaccttttattttctactcttttcagaggggttttctgtttgtttgggttttgtttcttgctgtgactgaaacaagaaggttattgcagcaaaaatcagtaacaaaaaatagtaacaataccttgcagaggaaaggtgggagagaggaaaaaaggaaattctatagaaatctatatattgggttgtttttttttttgttttttgttttttttttttgggtttttttttttactatatatcttttttttgttgtctctagcctgatcagataggagcacaagcaggggacggaaagagagagacactcaggcggcagcattccctcccagccactgagctgtcgtgccagcaccattcctggtcacgcaaaacagaacccagttagcagcagggagacgagaacaccacacaagacatttttctacagtatttcaggtgcctaccacacaggaaaccttgaagaaaatcagtttctagaagccgctgttacctcttgtttacagtttatatatatatgatagatatgagatatatatataaaaggtactgttaactactgtacaacccgacttcataatggtgctttcaaacagcgagatgagtaaaaacatcagcttccacgttgccttctgcgcaaagggtttcaccaaggatggagaaagggagacagcttgcagatggcgcgttctcacggtgggctcttccccttggtttgtaacgaagtgaaggaggagaacttgggagccaggttctccctgccaaaaagggggctagatgaggtggtcgggcccgtggacagctgagagtgggattcatccagactcatgcaataaccctttgattgttttctaaaaggagactccctcggcaagatggcagagggtacggagtcttcaggcccagtttctcactttagccaattcgagggctccttgtggtgggatcagaactaatccagagtgtgggaaagtgacagtcaaaaccccacctggagcaaataaaaaaacatacaaaacgtactggtgctttcctgt

SEQ ID NO: 30 - RNA, 3473 nt, Homo sapiens
  source 1..3473: mol_type = mRNA
acttccagagcttcagggccgcggctcacacctgagcgcgactgcagaggggctgcacctggccttatgggctgagaagaaagccaaggtcattgcaggaatgaatgctgtggaagaaaaccaggggcccggggagtctcagaaggtggaggaggccagccctcctgctgtgcagcagcccactgaccccgcatcccccactgtggctaccacgcctgagcccgtggggtccgatgctggggacaagaatgccaccaaagcaggcgatgacgagccagagtacgaggacggccggggctttggcattggggagctggtgtgggggaaactgcggggcttctcctggtggccaggccgcattgtgtcttggtggatgacgggccggagccgagcagctgaaggcacccgctgggtcatgtggttcggagacggcaaattctcagtggtgtgtgttgagaagctgatgccgctgagctcgttttgcagtgcgttccaccaggccacgtacaacaagcagcccatgtaccgcaaagccatctacgaggtcctgcaggtggccagcagccgcgcggggaagctgttcccggtgtgccacgacagcgatgagagtgacactgccaaggccgtggaggtgcagaacaagcccatgattgaatgggccctggggggcttccagccttctggccctaagggcctggagccaccagaagaagagaagaatccctacaaagaagtgtacacggacatgtgggtggaacctgaggcagctgcctacgcaccacctccaccagccaaaaagccccggaagagcacagcggagaagcccaaggtcaaggagattattgatgagcgcacaagagagcggctggtgtacgaggtgcggcagaagtgccggaacattgaggacatctgcatctcctgtgggagcctcaatgttaccctggaacaccccctcttcgttggaggaatgtgccaaaactgcaagaactgctttctggagtgtgcgtaccagtacgacgacgacggctaccagtcctactgcaccatctgctgtgggggccgtgaggtgctcatgtgcggaaacaacaactgctgcaggtgcttttgcgtggagtgtgtggacctcttggtggggccgggggctgcccaggcagccattaaggaagacccctggaactgctacatgtgcgggcacaagggtacctacgggctgctgcggcggcgagaggactggccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtcttcatgaatgagaaagaggacatcttatggtgcactgaaatggaaagggtatttggtttcccagtccactatactgacgtctccaacatgagccgcttggcgaggcagagactgctgggccggtcatggagcgtgccagtcatccgccacctcttcgctccgctgaaggagtattttgcgtgtgtgtaagggacatgggggcaaactgaggtagcgacacaaagttaaacaaacaaacaaaaaacacaaaacataataaaacaccaagaacatgaggatggagagaagtatcagcacccagaagagaaaaaggaatttaaaacaaaaaccacagaggcggaaataccggagggctttgccttgcgaaaagggttggacatcatctcctgatttttcaatgttattcttcagtcctatttaaaaacaaaaccaagctcccttcccttcctcccccttcccttttttttcggtcagaccttttattttctactcttttcagaggggttttctgtttgtttgggttttgtttcttgctgtgactgaaacaagaaggttattgcagcaaaaatcagtaacaaaaaatagtaacaataccttgcagaggaaaggtgggagagaggaaaaaaggaaattctatagaaatctatatattgggttgtttttttttttgttttttgttttttttttttgggtttttttttttactatatatcttttttttgttgtctctagcctgatcagataggagcacaagcaggggacggaaagagagagacactcaggcggcagcattccctcccagccactgagctgtcgtgccagcaccattcctggtcacgcaaaacagaacccagttagcagcagggagacgagaacaccacacaagacatttttctacagtatttcaggtgcctaccacacaggaaaccttgaagaaaatcagtttctagaagccgctgttacctcttgtttacagtttatatatatatgatagatatgagatatatatataaaaggtactgttaactactgtacaacccgacttcataatggtgctttcaaacagcgagatgagtaaaaacatcagcttccacgttgccttctgcgcaaagggtttcaccaaggatggagaaagggagacagcttgcagatggcgcgttctcacggtgggctcttccccttggtttgtaacgaagtgaaggaggagaacttgggagccaggttctccctgccaaaaagggggctagatgaggtggtcgggcccgtggacagctgagagtgggattcatccagactcatgcaataaccctttgattgttttctaaaaggagactccctcggcaagatggcagagggtacggagtcttcaggcccagtttctcactttagccaattcgagggctccttgtggtgggatcagaactaatccagagtgtgggaaagtgacagtcaaaaccccacctggagcaaataaaaaaacatacaaaacgta

SEQ ID NO: 31 - RNA, 9421 nt, Homo sapiens
  source 1..9421: mol_type = mRNA
gcgcgggcggcggcggcggcgagagcagaggacgagccgggacgcggcgccgcggcaccagggcgcgcagccgggccggcccgaccccaccggccatacggtggagccatcgaagcccccacccacaggctgacagaggcaccgttcaccagagggctcaacaccgggatctatgtttaagttttaactctcgcctccaaagaccacgataattccttccccaaagcccagcagccccccagccccgcgcagccccagcctgcctcccggcgcccagatgcccgccatgccctccagcggccccggggacaccagcagctctgctgcggagcgggaggaggaccgaaaggacggagaggagcaggaggagccgcgtggcaaggaggagcgccaagagcccagcaccacggcacggaaggtggggcggcctgggaggaagcgcaagcaccccccggtggaaagcggtgacacgccaaaggaccctgcggtgatctccaagtccccatccatggcccaggactcaggcgcctcagagctattacccaatggggacttggagaagcggagtgagccccagccagaggaggggagccctgctggggggcagaagggcggggccccagcagagggagagggtgcagctgagaccctgcctgaagcctcaagagcagtggaaaatggctgctgcacccccaaggagggccgaggagcccctgcagaagcgggcaaagaacagaaggagaccaacatcgaatccatgaaaatggagggctcccggggccggctgcggggtggcttgggctgggagtccagcctccgtcagcggcccatgccgaggctcaccttccaggcgggggacccctactacatcagcaagcgcaagcgggacgagtggctggcacgctggaaaagggaggctgagaagaaagccaaggtcattgcaggaatgaatgctgtggaagaaaaccaggggcccggggagtctcagaaggtggaggaggccagccctcctgctgtgcagcagcccactgaccccgcatcccccactgtggctaccacgcctgagcccgtggggtccgatgctggggacaagaatgccaccaaagcaggcgatgacgagccagagtacgaggacggccggggctttggcattggggagctggtgtgggggaaactgcggggcttctcctggtggccaggccgcattgtgtcttggtggatgacgggccggagccgagcagctgaaggcacccgctgggtcatgtggttcggagacggcaaattctcagtggtgtgtgttgagaagctgatgccgctgagctcgttttgcagtgcgttccaccaggccacgtacaacaagcagcccatgtaccgcaaagccatctacgaggtcctgcaggtggccagcagccgcgcggggaagctgttcccggtgtgccacgacagcgatgagagtgacactgccaaggccgtggaggtgcagaacaagcccatgattgaatgggccctggggggcttccagccttctggccctaagggcctggagccaccagaagaagagaagaatccctacaaagaagtgtacacggacatgtgggtggaacctgaggcagctgcctacgcaccacctccaccagccaaaaagccccggaagagcacagcggagaagcccaaggtcaaggagattattgatgagcgcacaagagagcggctggtgtacgaggtgcggcagaagtgccggaacattgaggacatctgcatctcctgtgggagcctcaatgttaccctggaacaccccctcttcgttggaggaatgtgccaaaactgcaagaactgctttctggagtgtgcgtaccagtacgacgacgacggctaccagtcctactgcaccatctgctgtgggggccgtgaggtgctcatgtgcggaaacaacaactgctgcaggtgcttttgcgtggagtgtgtggacctcttggtggggccgggggctgcccaggcagccattaaggaagacccctggaactgctacatgtgcgggcacaagggtacctacgggctgctgcggcggcgagaggactggccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtcttcatgaatgagaaagaggacatcttatggtgcactgaaatggaaagggtatttggtttcccagtccactatactgacgtctccaacatgagccgcttggcgaggcagagactgctgggccggtcatggagcgtgccagtcatccgccacctcttcgctccgctgaaggagtattttgcgtgtgtgtaagggacatgggggcaaactgaggtagcgacacaaagttaaacaaacaaacaaaaaacacaaaacataataaaacaccaagaacatgaggatggagagaagtatcagcacccagaagagaaaaaggaatttaaaacaaaaaccacagaggcggaaataccggagggctttgccttgcgaaaagggttggacatcatctcctgatttttcaatgttattcttcagtcctatttaaaaacaaaaccaagctcccttcccttcctcccccttcccttttttttcggtcagaccttttattttctactcttttcagaggggttttctgtttgtttgggttttgtttcttgctgtgactgaaacaagaaggttattgcagcaaaaatcagtaacaaaaaatagtaacaataccttgcagaggaaaggtgggagagaggaaaaaaggaaattctatagaaatctatatattgggttgtttttttttttgttttttgttttttttttttgggtttttttttttactatatatcttttttttgttgtctctagcctgatcagataggagcacaagcaggggacggaaagagagagacactcaggcggcagcattccctcccagccactgagctgtcgtgccagcaccattcctggtcacgcaaaacagaacccagttagcagcagggagacgagaacaccacacaagacatttttctacagtatttcaggtgcctaccacacaggaaaccttgaagaaaatcagtttctagaagccgctgttacctcttgtttacagtttatatatatatgatagatatgagatatatatataaaaggtactgttaactactgtacaacccgacttcataatggtgctttcaaacagcgagatgagtaaaaacatcagcttccacgttgccttctgcgcaaagggtttcaccaaggatggagaaagggagacagcttgcagatggcgcgttctcacggtgggctcttccccttggtttgtaacgaagtgaaggaggagaacttgggagccaggttctccctgccaaaaagggggctagatgaggtggtcgggcccgtggacagctgagagtgggattcatccagactcatgcaataaccctttgattgttttctaaaaggagactccctcggcaagatggcagagggtacggagtcttcaggcccagtttctcactttagccaattcgagggctccttgtggtgggatcagaactaatccagagtgtgggaaagtgacagtcaaaaccccacctggagcaaataaaaaaacatacaaaacgtactggtgctttcctgtctaagtcgccttttgtgtgttcttttatgaggcccccaccatccacccctctgtgcacaaggcggctcccggcccctggaatgtgatgtttttggtcatctccaaaggctgcagttttatactgggaggctgatgacaccttttattataattattcttatggttctggttataattatgtttgttttaagattttctttaagaaaacaaaaacccaacacccttccctttaggtttcaaaccaaggtgcgggagcagggtgcttctgagttagcggtggctctggtgccctggctcccacccctcgggccaggtgggccactgggcatggtgacagctgggcggccagggagtccccaaggtctgccttctgggcttgtcactcttctcttctacttcccctcctcttgagtcgggtgtgtcagggctggagggaggaccggccgtctctctccttgtgtgtgtgattggagtggtgtgtatttcttttctagtacttggtctgatgatagctgtgctcttacctcgagtcagcagcacctggagccccaagtgctcaacactttgttctacttcctacaaggcaggcggcctggcgtcagtagtaggaagggatggcattgttggctccagggtgagggcccgacgggcaggtgcttgcctgggtagatggggtgtagatatgtggcatatagagatatatattttataatgggaggggggatggcacctcctgggactggtagagctgggaggtgtgtcggcaagcacatggccccaagtccacgtccctagaaagatggggcctacagtgataggcgctctataaatacatagatagggtcatgtttaagaaatattatggggtggggagtgggggaagcaggcttagtgtgctgagcctcggtccccactgcagacctaaaccccacaccaatcagtcaatctcacacgcatacacaggagcatatttctgtactgatgcataatgaatgcactcacacacacacacacacgccagggggctcccagcactcttctcccagggttcaggactttgtggagtcagcccgtgcagctatttacctctgttgccctgattgcaggtagactgtcaatcccagaagttgctaggtgctgaggcaggaaaaggaggaggtttcttttagcaggtgctctcctctagcaggtagtgttctatctcttttgcccctggtgatgtcaaaagattcttctgttagcaacctgtgatactgacctgcaactctaggcaccagtcaagtcagccctcaactccccctacctattctccccaactgaaatcaaattgacatccaggtctgtcttggccagggggcttccattggggaaaaaaaatgggacagtgaacctcacctattgaataaaccaaattagtaattagtgacaaaaccaaaccaaggctttggaccccctgctcattactcaggagtaaattcacacagctggtggccggtctcacccctttttggggtgcttggtcctggaccccaatctactgtctcctcgacagattggtgtttaagatcatgcccagttgcttcctgtcacctcttgacaggtggtgactgggctgctgtggggctggagcagcgaggaagggagcctctgccccctcagtggagtccctgtgctaactgtggtgctcacccagctcctccttgctcccgcggggctctgtccttcgggcctggagtccaccagcgccactccaggagggctgggggaggaggggctgcctcaggcagtccaaggtgctccctcccccgctggccaatgctaaggtcttcccggctcccagggtcaggacagagtcttctaaggctcaggctctcccaagccactgcttagtgacagttagcctttcccagcggcccaagctccaccggaactgggcctggtgctccaaccaaagggttggtgttgggacttgggaagagacttggtagccaagggcatggcaagcctgggggattcctgtgggccggcttgctcgaggagggagtgctgggcacagaggagcccttatggctgacagcttgagagcacttgttcctccagatctagccacttgaccaggccctagaatggaccagatgccccaggggtcatggcccccccccccccccccttgcagggacgagagttaacaatcaccagcttttcttccggcccagcctgtgtgcttggtctcacagtagaatttaggagagtgtggggctttgggattgtgccaacttggccaagctgagatatgcttaatccacctcaacgctggggcctctggggacaggtattgggtcaccttccaaggggtgctgaacttcagcaggctgtagataaggcagaggttagaggcagtggagtgggggaaccaagaaggcctagggaaaattgccaggcatattccctgtggaattttctttccgaagagtaggcccatttcccccttagaaacaagggttctgggggctgatgacagcttcccctgcaggtgccaggtggcgcatgatgccaaagagatccgtggctcttgcctgggggaggtagccaggtgctcgtgctggggacacagtccttggcctcctgcctaagcaggaagctagtttccagcaggcggctggtactaaccccccgccccacgggagcgacaggctggtgcaccggcagactggggggccgcaggcgggcactagcaggggtctgcagtccatagatgatgacatgaatctgttgctgtccttttgtctctaggccccagagacttcttgctattgaacatcctattggaaagcaaactttccaagagagcacccgggacctgttagcttttgaaacaggcctgggtgtgattgactggtagagatgcctgtgtgatttttggcgcattgcaggagtgtggattgggacaggcaccccagcaggaaggaaggctgacaccaaaggggtctcacagggcccagctccccttgcttcccgataattggccaccaggccatggcgactctggggaagcctagatgggaacacccaccttctccagcagtagaaggaatatttcagcagagccatctgggagatgttcttataggaaaactgcagtcattcatttcatagttttgcttatataaatagagtacatttgggaggcagttttgaattcctgttgctactctgcttggtgcggagttgcagtggccccaagtgtctttcagagctgttgcttttaggatccccctcaagctgtgggttagggccagggcagggagggggtgctgcctggcactgtgccctgaagagggctgcttgcttcctttggtcgggagcacggcagagccaacattctggtgggaaccgagtttctagccaggcccctgttttggtcccgggaggtgagtaatctgagtttgtcattgcgtattctccacgctgcctgctctcagtgctggagactagagagcgaccctggagttgtcctctgggagtcctgcctgtttgccagggagcagctgaaacctccccgctcccaatcccgggggtggaaaaggtgtagtggaggaatggtctggagctgggatggcccttctggccatgccctgctgagccaggattaaggaaagtgtcagccgcctccttaccaggccctgtgctggggtcaggaggaccgggaggagagggaaggggtgagaggatggactctgctgttgtagtgtgagctctggaatctttacgttttggtaaaggcaggccagggtctttgaggatcgagttggggaaccttgcctctgtgtttccgagacccagctaaggtgggcctttggtgggtgacctgtctctgacccgaggcagcagagttttgggggagggagaaacaagcaaggagcagggaggacaaggacacaagatccctttgctggtggtgacttactcacttcccccaccagagggcctgggactcccgaaactcccgtaactggaaagctccctggctcagctccactggggggtgaggagggcggctccctcagccccagcctagcggcctccaaccctgtgcctgagaacatgagcagcggtctaaagcatcccctagtcccatttcacttcacagaccgatttggttgcacagccctagaattcaacagacaacaaaaatgctgaaattccttagaatacgcagagggaggaggtgactcaccaaggtgctaaaacattttattaaaaatatatattgttaaattaagtctgctgtctggacaatgactgtttgttttgttttcttgtagtggagtttaaaagagactattattttactctgatatattattattaaaaaggcattttaactttgtacttgaaaactaaatgagcgatttcatgtgttttagctgaggcattgaagtagcgggtgcttacttatttgtgggaaatcatgtattcatactgaagaataaactccgtagctgatttggtaataacttttactgttacagacgatttcgctttgaccccggtggcagagcacttttactccacattccacgcagctaaatgcaggacttcccagactcttccctccccagccctctttcctttctcttaagttcctcttttctcctttattattattatttttttttcctggttccagcatcctttcaccactgtattttttttttttttttttttttttttttttttttttttttttttttttttttagggttgcttctctatttattgacaataataatgtacatttcacagtctggttctaggacattcagggaagggcgagtgggggcccgaggggtccctgccccacccgcctggtgtcctgttgtatctctgtgtaagtgatgctcgtgacatagctgtttatgaaataattaaagaggtcaatgcgtacaacagatgtagaaagtctgtcagttccgccttcatcacatttttttttgtgcccagaagaatataataaagctcctttctaatgtacttgtgctggagaacacttgaataaatggactgtttttgtgcaaaaagaaaaa

SEQ ID NO: 32 - RNA, 3608 nt, Homo sapiens
  source 1..3608: mol_type = mRNA
ccctccgcccccagccccatcgcccccttcccctcccccaagacgggcagctacttccagagcttcagggccgcggctcacacctgagcgcgactgcagaggggctgcacctggccttatggggatcctggagcgggttgtgagaaggaatgggcgcgtggatcgtagcctgaaagacgagtgtgatacggctgagaagaaagccaaggtcattgcaggaatgaatgctgtggaagaaaaccaggggcccggggagtctcagaaggtggaggaggccagccctcctgctgtgcagcagcccactgaccccgcatcccccactgtggctaccacgcctgagcccgtggggtccgatgctggggacaagaatgccaccaaagcaggcgatgacgagccagagtacgaggacggccggggctttggcattggggagctggtgtgggggaaactgcggggcttctcctggtggccaggccgcattgtgtcttggtggatgacgggccggagccgagcagctgaaggcacccgctgggtcatgtggttcggagacggcaaattctcagtggtgtgtgttgagaagctgatgccgctgagctcgttttgcagtgcgttccaccaggccacgtacaacaagcagcccatgtaccgcaaagccatctacgaggtcctgcaggtggccagcagccgcgcggggaagctgttcccggtgtgccacgacagcgatgagagtgacactgccaaggccgtggaggtgcagaacaagcccatgattgaatgggccctggggggcttccagccttctggccctaagggcctggagccaccagaagaagagaagaatccctacaaagaagtgtacacggacatgtgggtggaacctgaggcagctgcctacgcaccacctccaccagccaaaaagccccggaagagcacagcggagaagcccaaggtcaaggagattattgatgagcgcacaagagagcggctggtgtacgaggtgcggcagaagtgccggaacattgaggacatctgcatctcctgtgggagcctcaatgttaccctggaacaccccctcttcgttggaggaatgtgccaaaactgcaagaactgctttctggagtgtgcgtaccagtacgacgacgacggctaccagtcctactgcaccatctgctgtgggggccgtgaggtgctcatgtgcggaaacaacaactgctgcaggtgcttttgcgtggagtgtgtggacctcttggtggggccgggggctgcccaggcagccattaaggaagacccctggaactgctacatgtgcgggcacaagggtacctacgggctgctgcggcggcgagaggactggccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtcttcatgaatgagaaagaggacatcttatggtgcactgaaatggaaagggtatttggtttcccagtccactatactgacgtctccaacatgagccgcttggcgaggcagagactgctgggccggtcatggagcgtgccagtcatccgccacctcttcgctccgctgaaggagtattttgcgtgtgtgtaagggacatgggggcaaactgaggtagcgacacaaagttaaacaaacaaacaaaaaacacaaaacataataaaacaccaagaacatgaggatggagagaagtatcagcacccagaagagaaaaaggaatttaaaacaaaaaccacagaggcggaaataccggagggctttgccttgcgaaaagggttggacatcatctcctgatttttcaatgttattcttcagtcctatttaaaaacaaaaccaagctcccttcccttcctcccccttcccttttttttcggtcagaccttttattttctactcttttcagaggggttttctgtttgtttgggttttgtttcttgctgtgactgaaacaagaaggttattgcagcaaaaatcagtaacaaaaaatagtaacaataccttgcagaggaaaggtgggagagaggaaaaaaggaaattctatagaaatctatatattgggttgtttttttttttgttttttgttttttttttttgggtttttttttttactatatatcttttttttgttgtctctagcctgatcagataggagcacaagcaggggacggaaagagagagacactcaggcggcagcattccctcccagccactgagctgtcgtgccagcaccattcctggtcacgcaaaacagaacccagttagcagcagggagacgagaacaccacacaagacatttttctacagtatttcaggtgcctaccacacaggaaaccttgaagaaaatcagtttctagaagccgctgttacctcttgtttacagtttatatatatatgatagatatgagatatatatataaaaggtactgttaactactgtacaacccgacttcataatggtgctttcaaacagcgagatgagtaaaaacatcagcttccacgttgccttctgcgcaaagggtttcaccaaggatggagaaagggagacagcttgcagatggcgcgttctcacggtgggctcttccccttggtttgtaacgaagtgaaggaggagaacttgggagccaggttctccctgccaaaaagggggctagatgaggtggtcgggcccgtggacagctgagagtgggattcatccagactcatgcaataaccctttgattgttttctaaaaggagactccctcggcaagatggcagagggtacggagtcttcaggcccagtttctcactttagccaattcgagggctccttgtggtgggatcagaactaatccagagtgtgggaaagtgacagtcaaaaccccacctggagcaaataaaaaaacatacaaaacgtactggtgctttcctgt

SEQ ID NO: 33 - RNA, 4395 nt, Homo sapiens
  source 1..4395: mol_type = mRNA
gcagtgggctctggcggaggtcgggagaactgcagggcgaaggccgccgggggctccgcgggctgcggggggaggcacttgacaccggcccggggagaggaggggccgctgtccctgcggccagtgctggatgcggggacccagcgcagaagcagcgccaggtggagccatcgaagcccccacccacaggctgacagaggcaccgttcaccagagggctcaacaccgggatctatgtttaagttttaactctcgcctccaaagaccacgataattccttccccaaagcccagcagccccccagccccgcgcagccccagcctgcctcccggcgcccagatgcccgccatgccctccagcggccccggggacaccagcagctctgctgcggagcgggaggaggaccgaaaggacggagaggagcaggaggagccgcgtggcaaggaggagcgccaagagcccagcaccacggcacggaaggtggggcggcctgggaggaagcgcaagcaccccccggtggaaagcggtgacacgccaaaggaccctgcggtgatctccaagtccccatccatggcccaggactcaggcgcctcagagctattacccaatggggacttggagaagcggagtgagccccagccagaggaggggagccctgctggggggcagaagggcggggccccagcagagggagagggtgcagctgagaccctgcctgaagcctcaagagcagtggaaaatggctgctgcacccccaaggagggccgaggagcccctgcagaagcgggcaaagaacagaaggagaccaacatcgaatccatgaaaatggagggctcccggggccggctgcggggtggcttgggctgggagtccagcctccgtcagcggcccatgccgaggctcaccttccaggcgggggacccctactacatcagcaagcgcaagcgggacgagtggctggcacgctggaaaagggaggctgagaagaaagccaaggtcattgcaggaatgaatgctgtggaagaaaaccaggggcccggggagtctcagaaggtggaggaggccagccctcctgctgtgcagcagcccactgaccccgcatcccccactgtggctaccacgcctgagcccgtggggtccgatgctggggacaagaatgccaccaaagcaggcgatgacgagccagagtacgaggacggccggggctttggcattggggagctggtgtgggggaaactgcggggcttctcctggtggccaggccgcattgtgtcttggtggatgacgggccggagccgagcagctgaaggcacccgctgggtcatgtggttcggagacggcaaattctcagtggtgtgtgttgagaagctgatgccgctgagctcgttttgcagtgcgttccaccaggccacgtacaacaagcagcccatgtaccgcaaagccatctacgaggtcctgcaggtggccagcagccgcgcggggaagctgttcccggtgtgccacgacagcgatgagagtgacactgccaaggccgtggaggtgcagaacaagcccatgattgaatgggccctggggggcttccagccttctggccctaagggcctggagccaccagaagaagagaagaatccctacaaagaagtgtacacggacatgtgggtggaacctgaggcagctgcctacgcaccacctccaccagccaaaaagccccggaagagcacagcggagaagcccaaggtcaaggagattattgatgagcgcacaagagagcggctggtgtacgaggtgcggcagaagtgccggaacattgaggacatctgcatctcctgtgggagcctcaatgttaccctggaacaccccctcttcgttggaggaatgtgccaaaactgcaagaactgctttctggagtgtgcgtaccagtacgacgacgacggctaccagtcctactgcaccatctgctgtgggggccgtgaggtgctcatgtgcggaaacaacaactgctgcaggtgcttttgcgtggagtgtgtggacctcttggtggggccgggggctgcccaggcagccattaaggaagacccctggaactgctacatgtgcgggcacaagggtacctacgggctgctgcggcggcgagaggactggccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtcttcatgaatgagaaagaggacatcttatggtgcactgaaatggaaagggtatttggtttcccagtccactatactgacgtctccaacatgagccgcttggcgaggcagagactgctgggccggtcatggagcgtgccagtcatccgccacctcttcgctccgctgaaggagtattttgcgtgtgtgtaagggacatgggggcaaactgaggtagcgacacaaagttaaacaaacaaacaaaaaacacaaaacataataaaacaccaagaacatgaggatggagagaagtatcagcacccagaagagaaaaaggaatttaaaacaaaaaccacagaggcggaaataccggagggctttgccttgcgaaaagggttggacatcatctcctgatttttcaatgttattcttcagtcctatttaaaaacaaaaccaagctcccttcccttcctcccccttcccttttttttcggtcagaccttttattttctactcttttcagaggggttttctgtttgtttgggttttgtttcttgctgtgactgaaacaagaaggttattgcagcaaaaatcagtaacaaaaaatagtaacaataccttgcagaggaaaggtgggagagaggaaaaaaggaaattctatagaaatctatatattgggttgtttttttttttgttttttgttttttttttttgggtttttttttttactatatatcttttttttgttgtctctagcctgatcagataggagcacaagcaggggacggaaagagagagacactcaggcggcagcattccctcccagccactgagctgtcgtgccagcaccattcctggtcacgcaaaacagaacccagttagcagcagggagacgagaacaccacacaagacatttttctacagtatttcaggtgcctaccacacaggaaaccttgaagaaaatcagtttctagaagccgctgttacctcttgtttacagtttatatatatatgatagatatgagatatatatataaaaggtactgttaactactgtacaacccgacttcataatggtgctttcaaacagcgagatgagtaaaaacatcagcttccacgttgccttctgcgcaaagggtttcaccaaggatggagaaagggagacagcttgcagatggcgcgttctcacggtgggctcttccccttggtttgtaacgaagtgaaggaggagaacttgggagccaggttctccctgccaaaaagggggctagatgaggtggtcgggcccgtggacagctgagagtgggattcatccagactcatgcaataaccctttgattgttttctaaaaggagactccctcggcaagatggcagagggtacggagtcttcaggcccagtttctcactttagccaattcgagggctccttgtggtgggatcagaactaatccagagtgtgggaaagtgacagtcaaaaccccacctggagcaaataaaaaaacatacaaaacgtactggtgctttcctgt

SEQ ID NO: 34 - RNA, 1808 nt, Homo sapiens
  source 1..1808: mol_type = mRNA
gcagtgggctctggcggaggtcgggagaactgcagggcgaaggccgccgggggctccgcgggctgcggggggaggcacttgacaccggcccggggagaggaggggccgctgtccctgcggccagtgctggatgcggggacccagcgcagaagcagcgccaggtggagccatcgaagcccccacccacaggctgacagaggcaccgttcaccagagggctcaacaccgggatctatgtttaagttttaactctcgcctccaaagaccacgataattccttccccaaagcccagcagccccccagccccgcgcagccccagcctgcctcccggcgcccagatgcccgccatgccctccagcggccccggggacaccagcagctctgctgcggagcgggaggaggaccgaaaggacggagaggagcaggaggagccgcgtggcaaggaggagcgccaagagcccagcaccacggcacggaaggtggggcggcctgggaggaagcgcaagcaccccccggtggaaagcggtgacacgccaaaggaccctgcggtgatctccaagtccccatccatggcccaggactcaggcgcctcagagctattacccaatggggacttggagaagcggagtgagccccagccagaggaggggagccctgctggggggcagaagggcggggccccagcagagggagagggtgcagctgagaccctgcctgaagcctcaagagcagtggaaaatggctgctgcacccccaaggagggccgaggagcccctgcagaagcgggtgagtcctcagcaccaggggcagcctcttctgggcccaccagcataccctgagagtcagggacttggctctccagcaggtcccaggaaggatggtctgggtcgtggctaaaggtctgcttgccaaggctatggcctggaggctactggctggatgcagcctgcgcatatgttttatttggcccatagagtgttttaaacatttaaaaaattagttgccagtatttaaaaatcaaaaaatttcacataaaaatctggagttttggcttctcatgaaaaaaaaaaaagctagatctggcaacagcgggctttcataacgccaacgattgctagactgggataatggcggtccctccatcgccttctgtggctggttgtgggccttagttttctgcagctctacctggcctgcttactctcccacgtgccatgcagttcctgggggttgctgtatttgtagcccctggcctgggcactcaagggcagcagataccctgtttgcctccctgagtgcagaggtcctgagcccaccctagttgggctgactcaactggaaatttggttgtgacagtggcgtggggagagggctgggtgattgtattctgtgtactgcccagcccaggcctcttcatctggggactttttggcctaaccctggaagcctggaaagttgcccacttttctctttcaggttaagccagcaatttcagggccaaccgagctgtaaacatgttagtaatgaggacaactagcatttgtacagggcttcacagtttacaaagcgctttctcatacattatcacatttgatcctcccagggccctgccaggttgttttgcatatgtgcattttaatttcaaaaagtcttccttccaagcgtgtatgatgaaatgagtaaattgattaattggcgtaacttattttgcatggatccaacctaatgttcatgcaggatagagaacatttccagaatacaaatttccaaacttaaaaaaaaaaaaaaaaaaaaaaaaaaaaaaaaaa

SEQ ID NO: 35 - DNA, 18845 bp, Homo sapiens
  source 1..18845: mol_type = genomic DNA
gggtttctgcagagtctacttcagaagcggaggcactgggagtccggtttgggattgccaggctgtggttgtgagtctgagcttgtgagcggctgtggcgccccaactcttcgccagcatatcatcccggcaggtaacctcaggctccaaggggcggccccggtccctggctgtggaggggtggctctaattccgcagaaggcaggaatggggtaaaggaaaaaagcgcagatcttgggtgtggagggagtcaaggcctgcgtgcaaagaagggaacaaatccaggtattgagaatgggaaaagaaaagggaaggaggggatgctggatgcttggatatgcagaaggtctgcttttgggcctgggagggagaaactgggcggtgaggaagttggatgcaacgacaaggggaaaatcaggtctgcacttgggttggaaaatcgaggggtggaagttgcaagaatgcatgtagaagactaagcctggagtgtaggaggaagaaaacgtgcagcagctgggggctggatgcaaagaaggctgtaagccgcagacgggcgtctgcaggcaactcaggcggcgaggaatagccttgaagccgatccctcgcgtccgccagggggcgtggaggggcggagctccagcggccttcagaggggtcgctaggccacacggggcgagagggggctgggggaaacacccccgccagttccgggtgcctggagtttaaaaggtcccagcaccagcgaggggaggggaggggaggggcgggcgtggagacctggaaggaggtaccgatccagccttgattgcagccctcttgcacaacggccataaggacgggtgcaggccaagagcaaggacatgcaaaccccaagacctgctaacgggcggcgaagcgggggcacgccctcgcacacgcagagataaattgtgctcccatgacctttatttggaaagtgcctgcgggcctaaaattggcctttgtcccaccgagtacactcagcactgtactttaaaccggataaactgggctgtctggcaggtactaaaaacagctaaattctcctgtgagtgttctgtatgttaacacttttattccttgttttgttttaggcgataaactacattcagttgagtctgcaagactgggaggaactggggtgataagaaatctattcactgtcaaggtgagaattagcaaattttcccccttcttctgtactttctttgatgtggcagtacagtgtttaactggtcttgggggcaggccgaggcactgcaagggaatgagtgaggaatatttccaacaacgaattccaacagcttccttgtttcttttaatacagtgtcttatctgcaagagatcctttttgcccagaaattattgtagttcatgaactcggcaaaaaacaatctggccaaaaacttgaggttgatgaaaataatattaaatggctcttttttttcattaaactactctgtattgttattccaggtttattttctggaattgcacaattatgttaggagataggattcagctacttcatatgagctggtgcctgctggtcttggaacagcccttccttctgaagttgggattgggttggagacaaaatctgaggggcaacaaaggtggagaagtcagtccagtggggcctcaagcccatcccatggaaatcagatccagaagaaatacaattatgttccacatggtagtaacaataaagcttcactgttttagttaaaagatggtaaaaatttcatggctaagtaatgtgaggaaaacctgaagcgtctgaagtatgcattatgagataaagaaagattactgcagagagtacagttgctttaactgagaaggaaatagtgaactaaaaagatatcagtcttcacattttaggtacaatatttgacctctattaactgtatttgccatcaagaataaattgccatgtcatgactgttgcatcatactatatagggacatggccatggaagtgtattatcaggaaccagataagttcactgcctccttttttgctttgttttaaaacaaggataggtgtgacagtggaatcaaacatgaactgaatcatatgctttcagctgtactacaaattatacttgacattacaaatgggaagtaattgactcatccacaccattctgttcatttcttttagtctagttcagttccagaaggttcccccaaaggaatctcctaaacactagtaagaagaattctagaattgcgaatttgggaagtgaaaatgggcctggggattggaaatacatccaggcttgtcaagaaacagacgcttactgtctactaatcattacatttttatttaaagaccaaattactttagctcaatttttcaagaaagactattttaggcttcttatctaataattctttaaagtcaattctcttgacttttttagtccttaagagaataaaagcaatttgcagctgtgttgttggattaaaaaagagtttcgattagagcatcattagaaggaatagataacttttagcaggctcagagcttcttctgcctctagggacaagctgggacagtttcatttcttccaaagcgtccaaccttgcactttctgagaatcagattgatttgtatatgctacccccacaccctatccttaaagtgctgtcttcctaggacaatgcacagtttgtcctatcccactcactagcagcttacccttgcctgtgaacagccagaagaagatgacagggacatgcttgggtcctgggcagtggccctactgtatagagattcttctaattgagaaatcactttgttgtatttagccccacatctccaatgtctgtgttatctctaggaaaacagttccaccaggaattcttcgtctcgcttttagacttaaagtttccctgaatatgggtcacaggaaccagctgaatctcctcatggctagttatttttattaaataacaaaaacagcctcttaaaggaggagaaatgctagcactcaccattctggacatcttgagagcccagtaaaatgatgagttggactgaagtgttcaataaacatgagattgtggaaataatctcatttcttaagacaaaccagtagttgagaataatgattgcattatgaagggcacgttatagggtcaggctctgccacgctctagctaggtggccttgggaaagctcttttttttttaacaaagtaaaatggggaaatgatggccatttttttggattgttatgagggctagctcagaaaccgcgtgtgaaacataacacaatactttgttcagagaagatactcaattctaatggtaaatagaattgctgttactagctttttattcaactacacataaattgaattgcatgaattgttcttttagtagtcactacgttgtttatataaacactacgttgtttatatatttcactttataatgaaataaatgacaaggcatgtattttttttttctttttaggtttattgaagtcaaaatgtccaaaaaaatcagtggcggttctgtggtagagatgcaaggagatgaaatgacacgaatcatttgggaattgattaaagagaaactcatttttccctacgtggaattggatctacataggtaaatgagttacccctccgtgtagcaaactcagaaaggataatctggctgggcatggtggtgcacacgtgaagtcccagctacttaggaggcagaggcaggaggatccctcaagaccaggaattcaagtccagcctgggcaacagagtgagaccccatcactttaaaaaaaaaaggaaaaaaaagggaaggagaaacaggaaaaaaaaaaagaatataatctgaattattttgaggtgaagttagctttttttatatagatatagctgtgttttgtgaataaagtttgttttaagcctaaatatattcagatgaatcttctaattttaacatactgctagaaaaactggatttatcatctaaaagtcaagaattataagggaaagaatgattttcagattcttctaaccttctgtttcctgtgaaggttttatttgtaataaaacagtggctaagaatagtacaatgtattgagaaaagggaaataagagaggatgtaagaaacagtaaactaaatgcagttaacacaattttgtcatttcaaactcaattttctatcactttacttagtcaatggacctgtttccttttcatgcctcaccttttgcccttttcatacaattgtagtgcagaaagaattcatgatgtttcttagttgaacccctcctttccagcaaaattcccttaaatcctgtgtccctttgccctgtgtgtaaagtgcctggggacagatgcaaaagtctatgtaattctctcatttttttcgctgtgtttttcataaacttgcttctgttctttgtacctttaggtagagatagaatttttaaaagggtaaagtggctgaagactggcatagaaaaatattgagcaacacgtttcagccaaactattggcaactctaagtgtgtttgcctcagcccagaattagtcatcaaaatttgtcctaaggacctgtttgagtggagacatttaattcacataatgtcattaaacatactttttaattgttctaaacctaagctgctgatgtctatattgaaatcatgataagatataaatacttgcagaagtcaaccaaacataagaaattaggcataaaaacatcttccaactaggttaaaaaaaaaaagcatcttaggaactcctgtttcaggactttacatgacagtaggtgaactggcagggttcttttccttataatggctgcaactaaagatgctttgagcctctttagtgtggaatcatgaggagttttaaaggggagtcactatgactaggaggaaaaaatttggtggctgcagactagataatttttggaggctcaaacaagttttaaatttttccaaaattcctattattatcactgactggttctttttcaggagaatggagcaggtagtttgttttatgtaggctggacagtggtctgggcaatttgttccttcctttcttatcttcttagaaatgttaagaagacaagcaaaaaatctaataatttgctgatagattctccttatgagaaatggttcctaggaacttgaaaactactgatcatacttcctgttggagttgggattggatcaacatttcctatgcaagaagataacaaaggacatgactttgttagatttatatgaattatctaaagtcgaagcagttggaagttatttttatttggaaatatgtcaagattactatctctttgtttgcatcatgagttgggtaggagatctaagaaaggaagataagggaaccaaaaagaaatctagaggcaggttttaagagctgaaaaaccttttaaatccttggagaacagaataaacaaaacattattttgttttcttgctttttaacaaactattgcatgcttcaaataaagctttgcagtcattttctatgctccctggacaactaacattctttcagctctgaatgacctgcaatctttgtgttggtgtatattaagctattactctctggtatgtagaaaaccattgatggattcctttacttacccaataaattgtaggcttgtttccagagcaaacacttgctcaagaattacagaaggaattagctgggcatggtggtgcatgcctgtagtccccgctactggggaggctgaggcgggggaatcacttgaacccaggaaggcagaggttgagtgagccaagatcgtgccactgcactccagcctgggcaacaaaacgagactcatctaaaaaaaaaaaaaaaagggaaaggctcattttatgactaactggcattaatgaagtagtcattcgcatttttggcatcaactgtcacatctttacctcccaccacttcaacgtcaaaatacaaaaaatcctttaacccctcccacccccttttatttttaaagagcaatatttactaagggaaaaaggtggtccttagacttacacttattatactaattatatggagagaaaccaggccattattgcctctatctggtgaaaaacaggaggcaaagggcagagtttgatggtttccattcattttcctgatagacagcttcccattacaagaggaggggcagcccagcagtctagcctctcttagttctctttgtagttggcacccatcttctgtgtttagggtgtgccagtgctaaaacttggcagattttggtgtactcagagccttcgctttctgcataatgagctctatatgccatcactgcagttgtaggttataactatccatttgtctgaaaaactttgcttctaatttttctctttcaagctatgatttaggcatagagaatcgtgatgccaccaacgaccaagtcaccaaggatgctgcagaagctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcgtcatgcttatggggatcaagtaagtcatgttggcaataatgtgattttgcatgttttttttttcatggcccagaaatttccaacttgtatgtgttttattcttatcttttggtatctacacccattaagcaaggtatgaaattgagaaatgcatatatgtataactgtatatttacacacatttagctaaaggcaaatacaaataaacttacaaataggcgtccatctcaacacattttttttcaaacatgctgttttttttcctttatccttttattcagttataccatatgatattgccatttttatgttggtaatttcatatggttcaaccagatctgtggttttcaacactggctgcacaataggatccccttacaagtttttttggtggttttgttttgctttgcttgatttgtttctttgttttagtttcaatgcttgagtaccaccctacacaaattaaaatctgaattccatggggttcaggcattttaaagctccccaggtgaatctaatgtgcaaacttgagaaccaccaaagattgtattaaacatgatcccatcatgcataaaagaaaaaactggctgggtactatggttcacacctgtaatcctagcactttggaggccaatttggaagcactgcttgaggccaggagtttgagactagcctggacaacatagcagaaccctgtctctacaaaaatgaaaagtatttcaataattatgtcaattgttcatggaagagccagttttgtttattcatatacaaagtgagtaggcgaagctgagtggggtggcatgtgcctatagtcccagctacttggaaggctgaggcctgaggatcccttaagcctaggagttcaaggttacagctatgagctatgatcacaccactgcctacgcaacagagcaagaccctgtctcgaaaaagaaaaagaaaaaaaaaactgactcggtagttgaaagcagccttactaaagcattccttcctgcctggaaaaaaagtattgcttctttacttctgtaccagtacacttgttgccaaattaagcaaaaaaactgccaagtaaacaaaatcaaagttcagtgttgaaactgaggtttcagactttcacagaaagtagtttatgtcttaaaaggaccttaaaaacaggaatctctcatgctgaaatccagaggttttaatgtagccatcattgaaaacagtgtgttgagggtaaacagtttagtccttgaggggtcagttaactttgttccactctgtggctaaaactatcctttcatcctggccaaagccttcagaaatagctcccaggggcccaagcaagggaaggtagatggcagtggtgcaaactcgctaatcttgtaaaaatcaccctagattctttccagttcagtcgtgccttttttctgcgtatatcctttttgttttttgtttttgagacagagtctcgctctgtcgcccaggctggagtgcagtggtgtgatcttggctcactgcaacctctgcctcccgggttcaagcaattctcctgcctcagcctcttgagtagctgggattacaggcacatgctaccatgcccggctaatttttgtgtttttagtagagacggggtttcaccatgttggtcaggctggtctcaaactcctgaccttgtgatccgcccgcctccgcctccgcctcccaaagtgctgggattacaggcatgccaccatgcccggccttcctctttttttttttttttttaattattttggcatcagtcatttgtaaaaatggccattctatttattaaagtccagaaagagaatttcaaacctcaggttgattcatggactgtttaaatatacataaacctatacaaagcatttatggtgctttggtacctcagtttctcccttttcagaaatagtaataaattaccacccatatttactttatggattttatatgtcaaaaatgttctgtgtaaagtaggtgtttaatctttattgttacttactgctcttttaccagttgttattagtatttctctacacttatagatgtggccggcacctgctggtacctaactctgcaagtatctgattcaattcattgaagaagatacaatgcagcaaagacttatggatgtctagtatatgccaggcatattattccactgagtagctgaggcccatgagggaggtgcctgtccttgaacagccctttcctaaaaatcatcatcatcccattgtagctgctcccagaatgatttgattgctccatatgaacaagtcctataacactagtaacaatgataccatgagagaccgagccccgatggcctaagtaaggacgagacctcaagtcctttgaccctggatgcattatttctactatatcatagttccctccttgtaagactgatgtaagaatcaaatgagattatgcaagttaagcatttaaagcatctggcatagagtaaggacaaagcattagctacctcagtactacaattagtattgttatcatcaccctctagggagacttccctgacttccttccatggtcacctcttttcctctgtcatcctatagtttttttttttttggggggggggtctaataccctcttttggcatttgaaagctcttatattgcttttcacttatatatgtctttatagggttgtaaacttcctaaaggaaagtggcttatgttattctctgtcctcttcaaatcagtgctcagaaaactttcctatacctcgtataggcacctaataaatatttactgatttaggggaatgtctggacctcttcatccccatctcctaaaaaaaaaaaaaaaaaaagaagtttgacatactgccctatttgtctaggtgtcttctaggtctatattatatgaatattctccttacaattcctgctagggtattttttttttctttatacctttttcccctctacacagtacagagcaactgattttgttgttcctgggcctggaaaagtagagataacctacacaccaagtgacggaacccaaaaggtgacatacctggtacataactttgaaggtatgtatgactgtagcataagcttcttttttttttaaacaaatgatttagataatatttttattgctttttttgttagttatgtgactgcctaaaaatataatctgaatctacagtgtttttgcaaaacccgaaacttgacataaagaagtaatttgcttccataccttcctgtggcatgaataaaggcacaaaagggttacccacttaattggaaatgcctttaggcaaagacaaggagatgattatgcatttatcttaaagcacagagagaaggttggcagaaatctgctgtgcatgaattatatacattaaatattaatattctcaaatcacccagcatataatcctgccctttgctctggtagggctcttgtatctggaagaggtttccttatatatttgctgaagttttcttcttgcccttctgggtatttatcctgtgaaaaagtagaacatttgctttttgccttttctttcccagctaccctgctacctttgatggcaaatcccacccaaaagttgttgaaataattagtgggcccttttccttcactattgaagttgttgaaagcactcacacaaacaaaaatacccaacactcacctaccttttgtttttcttgcctcttccaaataatatctgttggaatctggttctgcaggcttcagcacctttcaccctgaacccaagttaaccctgagcattggcatggattcaagttctatctttaagtaataacaaagttcaataatagttgtaaaacacagtgattacatgtagcataattatttctacttatttctaccaactacagtaggagtgctcagtatcctccaggagattataagaattttcattcagtatttgaaatttaagtttttctcaataactgaggattgagtacacccctaaagtttaaaatagatgttgggaattgtaaaggtagggtgtttgtccattcttgcgttgctataaagaaatacctgaggctgcgtaagttataaagaaaagaggtttaattggctcatggttctgcaggctgtacaagcctggctccaacacctgttcctggtgagggcctcaggaagcttccaatcttggcagaaggcaaacagacaataggtgtatcacatggcaagagcgggaacaagagaaagagaagggggaggtcccagactcttaaacaaccagatttcaagtgaattaactgagcgagaactcacgtaatcaccaaaggggatggtcctaagccattcataagggatccaaccccatgttccaatctcctcccactgggccccacctccaacactgggaatcacatttcaacatgagatttggaagggacaaatatccaaaccatatcagtatgtaaaagaagtttcaaaattatttgccttttcttaaatgtatttctggttcatatcagtttttggcatttctgcatcttcaggattttaaagactgtatgttttttcatttctgctgtccttccaaaacaagttagaatgtccttatctagaactggcttttctctcattatgtgcctcagaaatttgttggaaacctgtctgggacttctgaaatctagtctcagatgtgagagctgagaagtaggtcatttggttgtggtgggtgattttagccttattacaactacattacatttattcttctcccttttttcctctcactggcttctcctctacagaaggtggtggtgttgccatggggatgtataatcaagataagtcaattgaagattttgcacacagttccttccaaatggctctgtctaagggttggcctttgtatctgagcaccaaaaacactattctgaagaaatatgatgggcgttttaaagacatctttcaggagatatatgacaagtaactatagttcttttttaactttttctttattctcaagtaagctgacatttgcatatagagtatgtatctccctatccctatgattctgggtaaattgagtgaaaatcatcactgtctcttaaagataagggcaaaactgaggtttagcaggagtgaaacaaaaccactctgacttgcttttataagataaacatgttcttaaataaggtccccacatgaattgttttcagagccaggttctttaatgtttaaataaacgtacgctgttagtgataagttctaatttttcatgtgtgagtttctaagtcagacccatttccattctttctgaagcccattgttagaatttaaaattctgttggccaggcacggtggctcatgcctgtaatcccagcactttgggaggccgaggcgggcggatcacgaggtcaggagatcgagaccatcctggctaacacggtgaaaccctgactctactaaaaataccaaaaattagccgggcgtggtggcgggcgcctctagtcccagttactggggaggctgaggcaggagaatggcgtgaacccgggaggcggaggttgcagtgagccgaaatcacgccactgcactgcagcctgggcgacagagcgagactccgtctcaaaaaaaaaaaaagaaaagaaaagaaaaaaaagaatttaaaattctgttttagtggagtcatttgaacttaagtctaagtttataacaacactggcttccacagcacaggaggtgagcatgtgttaatatttaagattggcataactccctttaggtgcaagtgttcaggccaaaatgttcctgaggcattttgattcctcctcctgctgcccatctataccaagcccagaaactgtctggaatatattttagtttcctgaatgacaccaagaagtagaacagtcttttcaaaaatgtattttaaaaataagctgaatctcaagaatctgatctatagtataatgaaaactgaaaagtgaagtagtcattgggatactctactgtctcacttaattctcacggcttccctgcaaggtgggtaaaattgttcctacagatagtcaaattgagttttacagttagaaaatgattgggctaggatttgagccaatgtctgtcagattcctgagtttctgctacttctactaaaatatgctgcttcttgtgtgtccggtcttctgtttgggacaagcagatgatatccctaacaaaatcaattttctttattattattctcttttaccttttgtttcccaggcagtacaagtcccagtttgaagctcaaaagatctggtatgagcataggctcatcgacgacatggtggcccaagctatgaaatcagagggaggcttcatctgggcctgtaaaaactatgatggtgacgtgcagtcggactctgtggcccaaggtacagaggtggagcctggcctgtaggaacagggtcattccagggtagttctctaatttgggaaggggagttttgtagtcctttgttgtaaaatcttggaatgttaaacctccaggtccaccgtcttacctgacagctaaggaaattaagctcagactagagaaatgatttactcaagaccacatgggaagtaaattagtggattgacactcctggctccagtgtcactccttgaagttttaaagcctacctgatctgaaaatcactccctttccctgccctccttgtcctttggtgaaaatgtaaaaaaccagggcactcaggcaaggagggataaagtgaggagcttgatcatgtcataaccaagctttggtgcctgcaaagctcatttattgggcagcaggacctactcatgtccctctgccagctgctgttctggtagaatataaaacaaatgtgtttggctgggcacagtggctcacgcctgtaatcctagcactttgagaggccaaggcagtggatcacttgaggccaggagttcaagaccagcctggccaacatagcaaaaccccatctctacttcaaaaaaaaaaaaaaaaaaaaattagccaggtgtggtcgcatgcctatagtccctggcactcaggaggctaaagcatgagaatttcttgagcctgggaagcggaggttgcagtgagctgagatcacgccactatactccagcctgggcaacagagcaagactcttgtctcaacaacaacaaagaaaaagtgtttttgtttactgcttagcttacacacaaatcaattgagcaaagaaccctaggggctgtttacagaaattccaggagaagtttcatgcatggggtaagggccaaatcaacagaaacacagcgcctggtttcctcagcaccaagttgcccttcagtttcagttgcccttggtagctgtatgaacttgaatctccccagatgaatgtttgttaactttaattatgtttgtttttcccaagaagtactactttttctgtctcttatcagtgttaaacatctcctctcagtttgaaaatcagtagatcaacctggttaaagataactttttgccagataaatgagttacacaactctccttaccatccttctgtttctctacatcctcacaatcatttgtactgaatgcaacagaggaaggagtgttattgctttgtgatgactccgtggttgattattaacttcacaggtgatgttgggaggtagcaaaaatggagaaaagaagaaaatcaattatatagatataagagttgtaatgtggttctttcaataagaaaccacagaagagaactgttgtactgagggtatgaaagagagcgaggaagggcaactgggatgttcttagatccaaccgatcctgtgtttacctgccttctcttcccagtcccagcaagtgtatgagcagcttgagggttcttaagagacagctctcgtccatctttatttccccagaacagcagagcattgcacataagaataacaataatagattgagctctgtgtccagtactattctaaggaatataaatatatattcatatatatttatgaatatatgtatatttatctaatcccacagcaacctagtgaagtaggtgctgttattactcccattttacagataagaaaactgagccgcagtagctatgaagtcatagaactagagtggaatgtaggcagttggaccctgaaccctccatcaaggagtaggtatccctgaaataagtgaggggaaaatgcagcctttccagacccattggctctgatatctggagcggggatcctgccttctgaaaggcagaattattggtgattccatgtgctcttgtttactttgcttaccaagacctatcaagattgagtcatttatgctctcatctctgagagactcagtgctcttcatgcagttggacccaacgcttcatgctccactacttacagggtatggctctctcggcatgatgaccagcgtgctggtttgtccagatggcaagacagtagaagcagaggctgcccacgggactgtaacccgtcactaccgcatgtaccagaaaggacaggagacgtccaccaatcccattggtaagatggtgtttacattgatttctcaccagagagtgctctccaaagcagcccagatgtgttcccttggctgctcagtgttttgtgtgcaaagtcatcaccaagtagctgatttgttctactagtaagatctatttgaatatgcttcttaaaatatacaatatttgcttttctgttttgtgggagaacataggttacagagaggaatgttatcaacaagcattttcctttacctctggtgcagtgaaatgtcacataaggctacaaatctaaaagtcacatggatagatccttttaacaagtttcaaagatagcacttcaaaaagagattacagcctggaaaaaggagaccaatatagacaagagatagaagagattgtgacctttttcaaattagaaaatgccttaagtttcaaacactagtaccatgccatgaaaatgtgtttttttaaagtttctgtttggtaaaatatttctaactctcctaaaacttctagcccatgtggttagtatctaaagaagtctgtcacctaaacaaaaaactattctgttttggggaactatgagacatttggaaacctctataactaggatgattggagtgttgtgttttttttcccctcttttagcttccatttttgcctggaccagagggttagcccacagagcaaagcttgataacaataaagagcttgccttctttgcaaatgctttggaagaagtctctattgagacaattgaggctggcttcatgaccaaggacttggctgcttgcattaaaggtttacccaagtaagtataagatgccctagtttctatggtcaaattaacacttatcctcactgggctcagagcatcggtgctttctggaattatataaattgttattcatcatctagttcaggagtcagcagatatctgtaaattgtcagatagtgaacattttaggctttgtggaacatatagtctttttgcaactacacaactattcaactttgcctctgccattgtagcaggaaagcagtcatagacaatacagaatcaaatggtgtggctggtaccaatcaaacttttatgttcaaaaccagcctcgatttggttcatgtgctgggctgtaatttgctgacatcccatctagttgataatcattgaacgaaaaggttctatttaattttaagaagcagattatagacaaatagaaacaaagatacttcagaggctattttattctactcctttattttctttaacttactgatgagaaaagtgagcagcacaagtttaaggtgtgcaattgattacagtcccttaagtatctgtaaccaatatttgggcaaatatttgacagcccatatgtttactgagaatttttctgagaacatagtagccagtatcctggggggattgtctccatatctcaaccagtgcatctgcagaggtcccaggtagggtcagtataccccataatggggatatacttggtcactgcatgtttcttaatttaaatttccaacaagattgcagtgttcgctgggtgctgtggctcatgcctgtaatcctagcactttgggaggccgaggtgggtggatcacgaggtcaggagatcaagaccatcctggctaacacagtgaaaccccatctccactaaaaatacaaaaaattaaccaggcgtggtggcgggcacctgtagtcccagctactcgggaggctgaggcaggagaatggcgtgaacctgggaggcggagcttgcagtgagccgagatggtgccactgcactccagcctgcgtgacagagcgagactccatctcaaaaaaaaaaaaaagattgcagtgttcaaaatggtcttatcagtggaatacctttgtagcctatttaacaggcacctgtcagcaggattatgttatcctgtcttttgtttttctttttttttctttttattgagacagagtctcgctttgcccaggctggagtgcagtggcgtgatcttcgctcactgcaacctccgcctcctgggttcaagtgattcttctgcctcagcctcccaagtagctgggacttgtgccacaacacccggctaatttttttttttttttttttgtatttttagtagagacggggtttcactttgttagccaggatggtctccatctcctgacctcatgatccacccgccttggccccccaaagtgctgggattataggcgtgagccaccaggcccggcctgtcttttgtttttcttaagacactgatagaaataacaacattataactttttttaaggacatgtaacggtttgtgggaaggtgggtaaaggtttggctatgccctgaataagttttgttatgtttaagaatgaggaagaagtgtgatgattttggtctcagcagcattagtctgaaccgaaactcaatgataatttcacccccaaagctacatagcaagaaacctaactgagtatgtatggctgtctctactgccggttgcctagagagcctgagtactgagcctgggtcagccccaggaaccctgaacatagtcattgtctatagattttgtagcttactaacatgaatgcgttttcttccagaaacaaatagaatactattttaagtagcatttctaggactttaccactacctgctaccatatcagagaccaactaaattttcttttttttttccttcccgttagtgtgcaacgttctgactacttgaatacatttgagttcatggataaacttggagaaaacttgaagatcaaactagctcaggccaaactttaagttcatacctgagctaagaaggataattgtcttttggtaactaggtctacaggtttacatttttctgtgttacactcaaggataaaggcaaaatcaattttgtaatttgtttagaagccagagtttatcttttctataagtttacagcctttttcttatatatacagttattgccacctttgtgaacatggcaagggacttttttacaatttttattttattttctagtaccagcctaggaattcggttagtactcatttgtattcactgtcactttttctcatgttctaattataaatgaccaaaatcaagattgctcaaaagggtaaatgatagccacagtattgctccctaaaatatgcataaagtagaaattcactgccttcccctcctgtccatgaccttgggcacagggaagttctggtgtcatagatatcccgttttgtgaggtagagctgtgcattaaacttgcacatgactggaacgaagtaggagtgcaactcaaatgtgttgaagatactgcagtcatttttgtaaagaccttgctgaatgtttccaatagactaaatactgtttaggccgcaggagagtttggaatccggaataaatactacctggaggtttgtcctctccatttttctctttctcctcctggcctggcctgaatattatactactctaaatagcatatttcatccaagtgcaataatgtaagctgaatcttttttggacttctgctggcctgttttatttcttttatataaatgtgatttctcagaaattgatattaaacactatcttatcttctcctgaactgttgattttaattaaaattaagtgctaattaccatt

SEQ ID NO: 36 - RNA, 2313 nt, Homo sapiens
  source 1..2313: mol_type = mRNA
agggggcgtggaggggcggagctccagcggccttcagaggggtcgctaggccacacggggcgagagggggctgggggaaacacccccgccagttccgggtgcctggagtttaaaaggcgataaactacattcagttgagtctgcaagactgggaggaactggggtgataagaaatctattcactgtcaaggtttattgaagtcaaaatgtccaaaaaaatcagtggcggttctgtggtagagatgcaaggagatgaaatgacacgaatcatttgggaattgattaaagagaaactcatttttccctacgtggaattggatctacatagctatgatttaggcatagagaatcgtgatgccaccaacgaccaagtcaccaaggatgctgcagaagctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcgtcatgcttatggggatcaatacagagcaactgattttgttgttcctgggcctggaaaagtagagataacctacacaccaagtgacggaacccaaaaggtgacatacctggtacataactttgaagaaggtggtggtgttgccatggggatgtataatcaagataagtcaattgaagattttgcacacagttccttccaaatggctctgtctaagggttggcctttgtatctgagcaccaaaaacactattctgaagaaatatgatgggcgttttaaagacatctttcaggagatatatgacaagcagtacaagtcccagtttgaagctcaaaagatctggtatgagcataggctcatcgacgacatggtggcccaagctatgaaatcagagggaggcttcatctgggcctgtaaaaactatgatggtgacgtgcagtcggactctgtggcccaagggtatggctctctcggcatgatgaccagcgtgctggtttgtccagatggcaagacagtagaagcagaggctgcccacgggactgtaacccgtcactaccgcatgtaccagaaaggacaggagacgtccaccaatcccattgcttccatttttgcctggaccagagggttagcccacagagcaaagcttgataacaataaagagcttgccttctttgcaaatgctttggaagaagtctctattgagacaattgaggctggcttcatgaccaaggacttggctgcttgcattaaaggtttacccaatgtgcaacgttctgactacttgaatacatttgagttcatggataaacttggagaaaacttgaagatcaaactagctcaggccaaactttaagttcatacctgagctaagaaggataattgtcttttggtaactaggtctacaggtttacatttttctgtgttacactcaaggataaaggcaaaatcaattttgtaatttgtttagaagccagagtttatcttttctataagtttacagcctttttcttatatatacagttattgccacctttgtgaacatggcaagggacttttttacaatttttattttattttctagtaccagcctaggaattcggttagtactcatttgtattcactgtcactttttctcatgttctaattataaatgaccaaaatcaagattgctcaaaagggtaaatgatagccacagtattgctccctaaaatatgcataaagtagaaattcactgccttcccctcctgtccatgaccttgggcacagggaagttctggtgtcatagatatcccgttttgtgaggtagagctgtgcattaaacttgcacatgactggaacgaagtatgagtgcaactcaaatgtgttgaagatactgcagtcatttttgtaaagaccttgctgaatgtttccaatagactaaatactgtttaggccgcaggagagtttggaatccggaataaatactacctggaggtttgtcctctccatttttctctttctcctcctggcctggcctgaatattatactactctaaatagcatatttcatccaagtgcaataatgtaagctgaatcttttttggacttctgctggcctgttttatttcttttatataaatgtgatttctcagaaattgatattaaacactatcttatcttctcctgaactgttgattttaattaaaattaagtgctaattaccattaaaaaaaaaa

SEQ ID NO: 37 - RNA, 2470 nt, Homo sapiens
  source 1..2470: mol_type = mRNA
cgtggagacctggaaggaggtaccgatccagccttgattgcagccctcttgcacaacggccataaggacgggtgcaggccaagagcaaggacatgcaaaccccaagacctgctaacgggcggcgaagcgggggcacgccctcgcacacgcagagataaattgtgctcccatgacctttatttggaaagtgcctgcgggcctaaaattggcctttgtcccaccgagtacactcagcactgtactttaaaccggataaactgggctgtctggcaggcgataaactacattcagttgagtctgcaagactgggaggaactggggtgataagaaatctattcactgtcaaggtttattgaagtcaaaatgtccaaaaaaatcagtggcggttctgtggtagagatgcaaggagatgaaatgacacgaatcatttgggaattgattaaagagaaactcatttttccctacgtggaattggatctacatagctatgatttaggcatagagaatcgtgatgccaccaacgaccaagtcaccaaggatgctgcagaagctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcgtcatgcttatggggatcaatacagagcaactgattttgttgttcctgggcctggaaaagtagagataacctacacaccaagtgacggaacccaaaaggtgacatacctggtacataactttgaagaaggtggtggtgttgccatggggatgtataatcaagataagtcaattgaagattttgcacacagttccttccaaatggctctgtctaagggttggcctttgtatctgagcaccaaaaacactattctgaagaaatatgatgggcgttttaaagacatctttcaggagatatatgacaagcagtacaagtcccagtttgaagctcaaaagatctggtatgagcataggctcatcgacgacatggtggcccaagctatgaaatcagagggaggcttcatctgggcctgtaaaaactatgatggtgacgtgcagtcggactctgtggcccaagggtatggctctctcggcatgatgaccagcgtgctggtttgtccagatggcaagacagtagaagcagaggctgcccacgggactgtaacccgtcactaccgcatgtaccagaaaggacaggagacgtccaccaatcccattgcttccatttttgcctggaccagagggttagcccacagagcaaagcttgataacaataaagagcttgccttctttgcaaatgctttggaagaagtctctattgagacaattgaggctggcttcatgaccaaggacttggctgcttgcattaaaggtttacccaatgtgcaacgttctgactacttgaatacatttgagttcatggataaacttggagaaaacttgaagatcaaactagctcaggccaaactttaagttcatacctgagctaagaaggataattgtcttttggtaactaggtctacaggtttacatttttctgtgttacactcaaggataaaggcaaaatcaattttgtaatttgtttagaagccagagtttatcttttctataagtttacagcctttttcttatatatacagttattgccacctttgtgaacatggcaagggacttttttacaatttttattttattttctagtaccagcctaggaattcggttagtactcatttgtattcactgtcactttttctcatgttctaattataaatgaccaaaatcaagattgctcaaaagggtaaatgatagccacagtattgctccctaaaatatgcataaagtagaaattcactgccttcccctcctgtccatgaccttgggcacagggaagttctggtgtcatagatatcccgttttgtgaggtagagctgtgcattaaacttgcacatgactggaacgaagtatgagtgcaactcaaatgtgttgaagatactgcagtcatttttgtaaagaccttgctgaatgtttccaatagactaaatactgtttaggccgcaggagagtttggaatccggaataaatactacctggaggtttgtcctctccatttttctctttctcctcctggcctggcctgaatattatactactctaaatagcatatttcatccaagtgcaataatgtaagctgaatcttttttggacttctgctggcctgttttatttcttttatataaatgtgatttctcagaaattgatattaaacactatcttatcttctcctgaactgttgattttaattaaaattaagtgctaattaccattaaaaaaaaaa

SEQ ID NO: 38 - RNA, 2318 nt, Homo sapiens
  source 1..2318: mol_type = mRNA
gggtttctgcagagtctacttcagaagcggaggcactgggagtccggtttgggattgccaggctgtggttgtgagtctgagcttgtgagcggctgtggcgccccaactcttcgccagcatatcatcccggcaggcgataaactacattcagttgagtctgcaagactgggaggaactggggtgataagaaatctattcactgtcaaggtttattgaagtcaaaatgtccaaaaaaatcagtggcggttctgtggtagagatgcaaggagatgaaatgacacgaatcatttgggaattgattaaagagaaactcatttttccctacgtggaattggatctacatagctatgatttaggcatagagaatcgtgatgccaccaacgaccaagtcaccaaggatgctgcagaagctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcgtcatgcttatggggatcaatacagagcaactgattttgttgttcctgggcctggaaaagtagagataacctacacaccaagtgacggaacccaaaaggtgacatacctggtacataactttgaagaaggtggtggtgttgccatggggatgtataatcaagataagtcaattgaagattttgcacacagttccttccaaatggctctgtctaagggttggcctttgtatctgagcaccaaaaacactattctgaagaaatatgatgggcgttttaaagacatctttcaggagatatatgacaagcagtacaagtcccagtttgaagctcaaaagatctggtatgagcataggctcatcgacgacatggtggcccaagctatgaaatcagagggaggcttcatctgggcctgtaaaaactatgatggtgacgtgcagtcggactctgtggcccaagggtatggctctctcggcatgatgaccagcgtgctggtttgtccagatggcaagacagtagaagcagaggctgcccacgggactgtaacccgtcactaccgcatgtaccagaaaggacaggagacgtccaccaatcccattgcttccatttttgcctggaccagagggttagcccacagagcaaagcttgataacaataaagagcttgccttctttgcaaatgctttggaagaagtctctattgagacaattgaggctggcttcatgaccaaggacttggctgcttgcattaaaggtttacccaatgtgcaacgttctgactacttgaatacatttgagttcatggataaacttggagaaaacttgaagatcaaactagctcaggccaaactttaagttcatacctgagctaagaaggataattgtcttttggtaactaggtctacaggtttacatttttctgtgttacactcaaggataaaggcaaaatcaattttgtaatttgtttagaagccagagtttatcttttctataagtttacagcctttttcttatatatacagttattgccacctttgtgaacatggcaagggacttttttacaatttttattttattttctagtaccagcctaggaattcggttagtactcatttgtattcactgtcactttttctcatgttctaattataaatgaccaaaatcaagattgctcaaaagggtaaatgatagccacagtattgctccctaaaatatgcataaagtagaaattcactgccttcccctcctgtccatgaccttgggcacagggaagttctggtgtcatagatatcccgttttgtgaggtagagctgtgcattaaacttgcacatgactggaacgaagtaggagtgcaactcaaatgtgttgaagatactgcagtcatttttgtaaagaccttgctgaatgtttccaatagactaaatactgtttaggccgcaggagagtttggaatccggaataaatactacctggaggtttgtcctctccatttttctctttctcctcctggcctggcctgaatattatactactctaaatagcatatttcatccaagtgcaataatgtaagctgaatcttttttggacttctgctggcctgttttatttcttttatataaatgtgatttctcagaaattgatattaaacactatcttatcttctcctgaactgttgattttaattaaaattaagtgctaattacca

SEQ ID NO: 39 - DNA, 19424 bp, Homo sapiens
  source 1..19424: mol_type = genomic DNA
agcccgcggccaggcagccgggaggagcggcgcgcgctcggacctctcccgccctgctcgttcgctctccagcttgggatggccggctacctgcgggtcgtgcgctcgctctgcagagcctcaggctcgcggccggcctgggcgccggcggccctgacagcccccacctcgcaagagcagccgcggcgccactgtgagtgccgcgcggagggtccaggcaggcgcgcgcccccagctgcgacgtggcggcttcccaggctggggccgggaagcacgtcccgccagcccgcagcccggggacggcggggtcaggcgcacaaagggccgcggggcagcttcgctccgcgctcccccgcccggcgcttcccggccgtgccaggcgctggggtggggacttgagggacggtggcaactggtggcccgggcgacggccagctctgcgcgggcgggggctcggggctgccgcgcgcccctaggtatttcgggagggggtcctgagagtgtcccggacccgaagggtggcctgcccgccctgcgcccctggaacgagacgagcacccgtggggggctggagcaccccgcgcgctcccctggcgagagggagggtcgtggctcggcccctgctcagacaaaggctgggaggcgggagacatgcacttccccttccttttcagccaggcgcgcgctgataccaggcccacgtcagctatttttggagccttttacacgacagctggaggagcgtcctttttaattttccccttttgtttggccgcccccacccccaccccttcgccttcatcgctgcacttgaggctccatcctggggcctctccttgacttgacctgccttggcaggcacatgccctccctgcctggctcactcgccgcagagacctggcagcccgcgcaaaatgtcactttgcggaatcgttcccacggcttctgggtacccttagttccctgcttaggagggaagacagtagtcgggtcgtaataagcaagacttagcccgagcctccgttgccaacgcaggctgccttgcttggcgtgtgggcatcggcctgccccctcaccctggctacccaacacagctacaaaaggcagggaacaatgtaggtcccttggccctgcctaatgcctgttgccatggaaacccctatcctaatctggccaggagccccttgcagtgagccaggagagtgaggaagaggggatggggcccgctggccctgacctggccagaggaggtaatggttaaccggattgtgggagcagctgactagagccgggggggtagggaggcttgggccccagtcctaccttccctgccaaggagaaaggggcatgtctgcttttgtacctctgggaatctacctcagggatctgcccaacaactcccaggttccaagtgccccaggacccaaagattcccatatagcacaccaccttcagcaaaaatatggtggaagttagccacattctttaattctccctttttttctgccttgatggtaaaaaaaaaaaaaagaaacaaacaacccgaaaaacacacacaaacaggaacacaaagcgccttttaagggtcctgaatttggatacagaaaaagcagtcagttgcaggcccttctagaagtccccatgtgggttggttatgaatggggaaattgcttggtgttggagttggacatcagactctacagttgcgactctgccagcttgttacctggcaacgcagctggagacagaatacttggcattccccttgatgacaaggaaagaatattttggggctaaaagagccccgtgtctttgctgcctggtaattcctgcacgcatcttttcctattttgcaacgccataggcttccagcgactgctggtgatgtttctggtaagttagagcttggggcagtgcggaccagttctgtgaacggctcagtggcagaattacttatctagccttccttcactcctaacccagaaacttcttaattcttgccccttaagtgtgcagatctttctgccacgtcagataaggccctagctgccctgccagagctgattggggtggttgtgtttaggcctagtaggtactgaggaaaagcctctttggaatggctgaggcctgctcccacatcttctgtgtgaagttgagggcccttgggctggttccattcagggtatgaggtgctggaaactgctctcagattactggaagccagggtctgtggccttccctggggtttatactctgtaaacccatgttaaaaataccagcagattgcaaatattagtacactgtaggttgtaagcttcttcagggcatggggcctattgctggagagagatgccacccgtgtacatctggtgcaggctgctggtaagaactcactggcccctgtgtggcaatgcataccagatgggtgagaggggaggaaacacattcagtgaaacagaaggtgagggcagggcagccctgaccacagatggagttgacttcaacttgacctttatttatgtctttgacaaaagtagtatactcctactttgtgccagacaccatgccgggtgctggggatagagctgggaataaaaccagcagagactctttcaaaacccttaggagctcaaattctgatgtgggagggggcagacagtaagcaagatagatgttgactagtatgatatatgggtacaagagctaaggaaggccaggcactgtggcttacacctgtaatcccagaattttggggggctgaggtgggaggattgcttgaggctgggagctccagaccagcctgggcaatatagtgagaccccatctctacaaaaataaaaaataaaattagccaggcacgatggtgcatgtctgtggtcttagctactcaggaggatcatttgagcccaggaggtcaaggctgcagtgaggtacaattgtgccactgcactctagcctgggtgacaaagcaagaccctgcctcccccttctccccccaaaaagagaagagctaaggagaaacaggcaagaaaggtgtgtgcacaaaagtttgtatagggcagccagaaaagtcctcactgaaggaatcgctgaggaggggaggaagcaggccattttcggtattgggggaagagggactcgcaggtgccaagggcatgatggaagagcagagaggatggtgcgaaggctggctcagcaggcaccaggtggagtgacaggatgaggttcggcagtggggtctgtagagggctcacaggccatttttgagagctttcgcttttactttgcagggttttgagaggagtatcgttacctgatttccatcttatcgggagcattctagcttcagggacctggcaagagaggatgatggcttggacctgggtggtggcgctggggatggtgaagaagtggttggattctggatatattttgaaggaggagccagtagtggggaaaggttccaattcaggcgattttacataaaaatctagatacgcagcaggcactcacttatggcaagggtgggcttgtctgagggtggcttctggttttggtggtttggatgtgagagaagggaaggaatgaaggatgacaccaaggagtctactagagtttgttccaaaatggctgttctggccgcgtgcggtggctcccaggactttgggaggccgaagcaggcggatcagttggggccaggagttcgagacctgcctggccaccatggtgaaacccggtctctactaaaaatacaaaaattagctgggtgtggtggcaggcgcctgtagtcccagctactcaggggctgaggcaggagaattgcctgaacccaggaggtggagcttgcagtgagccgagatcacgccactgccctccagtgtaggcgacagagtgagactctgtctcaatacatacatacatacatacatacatacatgcatgcatacatacatacatacatacataaaatatacaaagttgctgtcctgccttcttgagcccccaggaattggaacctttccccactctctgatcttttgctgggacatgctgactgagcagcctttctgctgagagcatgggtaggaatgtggagggagcagttatgcctggggctggctgcctgcggctggacctctagcctcctgctgggagcagcaagaagccagggatgtgttttgaaatccttacttggtattgggtgctaggagccccagacagggaggagagtcagcctaacgtgcctctggtgactggtgtaattccatcctgcactctcgccaagaggacagtgtttgtgcaatgcatcagctgaagattcgtggtggctgtcacttcggaggttttcagggaggcctagctgctgctctgtgtatttcaggtagcttgtcaccacttatctctcagagaagggcagaccatggctgtatctccaccatacaggctgaacagcagacaggggtctcaaactccagtgccctgggccacactggagggcacttggctgtcatggagtgaagggagaggccgccttcagcccagcccatccttgccatgagtgagtgcctgctgcatttctagatttttatataaaatctcctgattttttttgactggtttaaatttttattattttggtaaaaaacgtataaaacttaccatcttagtcattgttcagtgtacagtattgttaagtacattcgcactgttgtgcagacattaccaccatccatcaccagaactttgtcaccttgcaaaaccgaaactctctacccatgaaacaatacctccccacttcccgtccccagtccctggcagccacctttttactttctgtttctatgaatttgactattctaggtacctcatgtaagtagaagcatatagtatttgtcttttgtgactggtttatttcacttagttagcataatgccccccaggttcatccactttgtagcaggtgtcagaatttcccttcttttcaaggctgaatcatattccattgtatgtagataacagtagtcccccttatccttggaggatgcatttcaagatccccagtggatacctgaaactgcaaagcctgtagatactatgtttttttcctatacatacagatctatgataaagtttaatttatgaattaggaacagtaagagattagcaataataactagtaataaaatagaacaattataacaatatactgtaatgaaagttacatgaatgtgatctcttaaaatatcttactgtactgtactcacctattttcagactatggttgactgtgggtaactgacaccacaggtaactgacgtcatggaaataaaacctttgataaggggggactgtatcacattgtctttatccattcatctgtgatggacacgtgggttgcttctatcttttgctgttgtgaacattgctgctatgaatgtgggcatacatatatctctttcagaccccgctttcgatatttttggatgtatatacagaagcagaattgctggatcatatagtaattcttgtccttttcttttctttttttttttcgagacggagtcttgctctctttcgcccatgctggagtgcagtggcgcgatctcagctcactgcaagctccgcctcccgggttcatgccattctcctgcctcagcctccccagtagctgggactacaggcgcctgccaccacgcctggctaattttttgtatttttagtagagtcagggtttcactgtgttagccaggatggtctcgatcttctgacctcgtgatccacccgcctcagccttccacagtgctgggattacaggtgcgagccaccgtgcccggcagtaattcttttccttttagctaaggaacacccatactgttttccacagcagctgcaccatttcacattcccaccaacagggcacaagagttccaatttctccacatccttgctagcactttattttctgttttgtttttcttttcctgatagtagctctcttagtggttgtgaggggatagtggggtgatatctcattatggttttgatttgcatttccctgatgattagtgatgttgagcatcttttcatgtgcttgttggccatttgtatatcatctttggacttgaaatgtctatttaagtcttttgtccattatatttatttatttattttgagatggagtcttcctgttacccaggctggagtgcagtggcatgatctctgctcactgcaacctccacctcctgggttcaagcaattctcctgcctcagcctcccaaatagctgggactacaggtgtgtgtcaccacaccaggctaattttttttctatttttagtagacacagagtttcaccattgtggccaggctggtctcgaactcctgacctcaagtgatctgccctcttcagccttccaaagtgctgggattacaggcatgagccacggtgcccagccagtcttctgtccattttaacgttgagttatttgttttattgttgagatttactgatttttaaaacttggtcaccaattaagatttttttaaaaaaccagtacctggaacaagcagaacaagtctctgctggccttggctcatgttgctgttctcagttcccctgggatgtttgcttccagggaggcctgggcactcctgactttggtgacagccctgcttgctctctctctgttacctgtaggtttccccttccacattcactgcctccgggcaggcactgttgtgttttaggcatttcagtatcccagtggggtactggataagtgcttagttagcacctgccggtgagtaaataatcaggtcctgtggtctctgagggtttgacacatcaggtctttgatcttgaccttgaactctcctagccatgcctgcctgaagcccaaccgtggagcccaggctgaggctgaaacctgaagaactggccccaagtgagtctctccttagggctcctgattatgatagttcatttttttttttttttaacaaatttgattagctgtcctcaaattaactcataggagatgctccccagaactaaactagctctgcaacagctcaacagactttccgcaggttctgcagctctttccactcaagcttcagaaaaaagtgaaggaatattgtagattgggttcaaaatccccatcgcgtcttgggaaaggcagtagagccagcagccagccaccttttgttctctagggaatggaatagtccaaggctgtgtttgtgttctgcttctctacatctgacccacgggtcttccagaggctgcatttgggtcagggacctgggctctatcttgcctctgatgcagaccacactctctgagtctcattattccccctaaatgatctctaagattatttttagattaaaaaaaaaaaacaaactctttgcatatctttggaaagtcagttgtttgaggtctgttaggctttaatcacttttgttttaaatgtgccgaggtggtttctaggcctgcttttcacaggtattctgtcccttgtcagttcttcccacccagttttttttgtttgtttgtttgtttttttgagatggagtctcgttctgttgcccaggctggagtgcagtgccgtgatcttggcttactgcaacctccacctcccgggttcaagcgattctcctgcctcagcctcctcggtagctgggattataggcacgtgccaccatgcctaatttttctatttttagtagagatggggtttcaccatgttggccaggctggtcttgaactccttaccttgagtgatccacctgcctcagcctcccaaagtgctaggattacaggcatgagccattacacccagccccaacccagtgttttcaaataatcattctcttcccttcacctgatttgctgtgtctaatctagagtggagctgaagacacatcaccacctctctaaggactgtgtgtaacacaagagtaattgggctgtaagtgaggctgaggagtgctgctttgaggaaagggcacagctctcagctgagctggtgagatttccctgtgactgaagttttattagatcttgttccctgctctgcatttgctgcttcttcccaggctgtgggcagaggagcaggctggtgccccggtgcccctatctgctgcaggctgaggttcaacaagccttgtccaaggcagctgtactgattcattgttaggtggccagttcctgagttttctttgaaattcacttcccagactgttgggtattcctgcgagtcagactctccttcagtagcgtcccctgccccctaccctccactggtctccctggacctctgatcagcccctcattgagtcctttgatgcttctctggtaggactgacccacctgcgtcggctgggatcaagggttctgagttccattcgcaggtgctgtaagcctgtccttcctgttgaatagggtcagagtcctcgtgactctgctgttttcaagctctgtaaccttgggcagtaagctgttaacctctctgagcctcagtttcccacacttaaaatggagataatggccaggcgtggtggctcacgcctgtaatcccagcactttgggaggccaaagtgggtggatcacctgaggctaggagttcgagaccagcctggccaacatggcaaaaccccgtctctactaaaaatacaaaaattagccgggcctggtggcgtgtgcctgtaatcccagctactcaggaagctgagacagcagaatcgcttgaacccgggaggcagaggttgcagtgagccgagatcacgccattgcattccagcctgggtgacagaccaaagactctgtctcaaaaaataaataaataaataaataaattaattaattaaataaaatggaggtaatgatacctccttggggttgtttttgggattaaatggaatcacattgtacagcattggtacactgtaaggcacatagtaggcactcaatacctatctatactatttcctttatcaccacaagttagttgagggctcttgggttgcacgcaacagacaaagttagtgactggttaacttggagaggattttattatgagctacacagcgtagctcatagaattgcagggaaaactataaagaactaagtcttggacaaaagtaatatcttggcatgggcctgtagccccagctactaaggaggctgaggtgcaaggattgcttgagccccggagtctgagcctggcttgggcaacagagagagataaagagaccctgactctaaacaataagtaaatacataaaagaggacccaggccctccttaggaactggatggcaggagcccctggacaacctcttactgatttggtgctggatgaggcagccgcagctagccagtgttctttgtgttgatctcctctgccttcatattccagggggagagggtctgacaggccagactggcccccaccctcttggccactggtgggctgggcatgtttttgggtagttctacctagccagattaaagtgggggtggggtggggagtccagccagcctgggcaacagggcaagactccatctctacaaaaaaattttaaaattaggctggcacggtggctcacgcctgtaatcccagcactttgcgggggtcaaggcaggtggatcacttgaggtcaggagtttgagaccagcctggccaacatggtgaaaccctgtctctactaaaaatacaaaaaattagccaggtgttgtggtgcacacctgaaatccccgctacttgggaggctgaggcccgagaatcacttgaactcgggaggcggaggttgcagtgggctgagatcatgccactgcactcccaccctgggggacaatagcgaaacttcgtctcagaaaaaaaaaaaattaaaaatagctgggtgtggtggcatgcgcctgtagtcccagctacttgggaggctgaggtcagaggatcacttgaagccaggaagtcaatgctgcagtaagctatgatggcatcagtgcattccagcctgaccaatggaacaggaccctgccaagaaagagagagagaaagggaggaaggaacgaaaggaaggaaggaaggaaagaaaggaaaggaaggaaagaaaggaaaggaagtaagtaaggatggatggatccaggtgctctaaaccagcagttcccaaagtttttggcaccaggggccagtttcatggaagacaatttttccacgggtggcagggtggtgggcagcggggatggttttgggatgagtcaagtgcattacatttattgtgcactttatttctattattattacattgtaatatataatgaaaaaattatatagctcaccataatgtacaatcggggggtcctgagcttgctttcctgcaactagacagtcccatctggaggtgctggaagacagtgacagatcatcaggcattagattctcataaggagtatgcaacctagatccctcgtgtgcgcagttcccaggcatgagctcaggtggtagtagtgctcactcacctgcagctctcctcctgctgtgtggcccagttcccaacagtccgtggacctgcactaaaggctgtggcctggtctgctgacctctctccagagcccacggtactagtccatggcctgggagttggggacccctgctctaagcagtgagggagctgatttgcagacgggggagtggacaccgggcagctggagccctggctttctgccactgtctcctggtgaagggcgggcctggctgggaggagagggcccccctccagcctccccaggggcatgatgcgctgtgtgtccctgcttctagatgccgacaaaaggatcaaggtggcgaagcccgtggtggagatggatggtgatgagatgacccgtattatctggcagttcatcaaggagaaggtagtgccccctcctgaagtgggtggctctccaggtgggctggccagggattgttctgtcccacagggtcttctggactgcaggtccctaggacccccccgttgtcctggtaggcagcagcagctctgtttctctccctctgtctacccttctttcacatctggtccactaattgttcagaagtcgtcaggcgggggcctgtccccagcagtccgtgttgtgatggtgccagatcccacacgttctgtcaactttgagaagcttcaaggttaatcctgtatcatttccatcttgatcccctacgttttgcctcacttcttgaaaaggagtgaaaagggaacagggctggtgtgtagggcagtgacccagcctgcagttgtgacgagccaccatggcaaaggggtcccagagtggccgcttggcttctccaaagttgccgtggtctatgtatgtccaggagagcatgtagtccttgtgaaggccccacactgtgtgtgcgttcatgggacaaaggttgagaactgtcacttccaactgtaagatctcaaagcacttgagaagaggaaaccagttgtatagagaaatctagatgtacttggggttggggtttggctgagttgatgggccatgtgaggggggcacacaggcacagtggaggaagaaccctccaaaagactgcggcctggcctgccaacctctctccaaagccccagcctctgccagctctggccaggccccactgagaaatggctgactccagctttctgctgcgcccctccactggccttcactcatccctgttttgactgactgtcccctgccatggcctgcagacttcttatcctgccctttgttgtcatgtccctgagtcactggggtgacgccttgctctggccctctgtccccagctcatcctgccccacgtggacatccagctaaagtattttgacctcgggctcccaaaccgtgaccagactgatgaccaggtcaccattgactctgcactggccacccagaagtacagtgtggctgtcaagtgtgccaccatcacccctgatgaggcccgtgtggaaggtgcgagggtgtggaggtgggcgggccagggagggtcacaggcttctcccaccggcctctccagacttggggtggggcagttggccatgtcactcatctcgggactgggaggttgggaggctccagagctggggctttcctgaggccaactgcacctgaggccagtctccctgtgtgtgcctggtggacagtggggcctcagaggtcacctttgatgaggctggggcaggcgtgggtaaggggctggcctcaggttccacggttagggcacagcagagctgagaacagaatccatgtcttccatctcccaggccagtggtgtttctgcttcactctggctctgacagtggtaggtgtcatcggtgacagggctgaggaggaggaggaggaggaaggtaatgctgatgtggtcacagtaaggatggcttctttgggcagtagtccattctggcgtggacagtcttgcaggcaggtatgcagagagattgtgcagaggagtgctgtgtgtaaggcctgtgtccttcagagcctcagcagcctctggggaacattgctgttgccctcctgccactggcttggccttgcaggtctgcctctgtccccatggtcatgcagggtcactgagaggtgcccccaccaggcctagactggcttctggtccttggctgttggggccgctctcgagggttccatgaagtcacagcagtgtgtgtgcagaggggccaggtttggataattcgagaaaggatggttgaggagttttctgagctctctcttcctactttccacagttggagggagctggcggaaagtactcgagatacatctgcaaattccccaggcctgcccctgtcctgcaggtcatgcctgacccacagcactgggatcatttgagtgttgagctcttcatttcacctgaaccttgggagttgtgtatccccatttatggacaaggcaggagagggtcacagccttaggaactggggtggcgttggggccaggaccggagcatgtggagtgggcagtgtgttgtcaggcttcgtcagacaagtccttcccccggactgtactccctccatgtgtgatgtgggaatcataatggccaacagagcacttagcatggaccgggcaccgttctaggccctttacacacgagctcatttcgtcctcacgacaacacttaggctggttatttttccagtaatttttttttacagctttggttgagccacttttgtaagcctcaccattaaaactggcagacttgctgggcatggtggctcacacctgtaatcccagcactttgggaagccgaggcgggtggatcacctgaggttgggaggtcgagaccagcctgaccaacatggagaaaccccatctctcctaaaaatacaaaattagcttggtgtggtgtcacatgcctgtaattccagctgctcgggaggctgaggcaggagaaccgcttgaacccaggaggcagaggttgtggtgagccgagatcgcgccattgcactccagcctgggcaataggagcaaaactctgtctcaaaaaaaaaaaaaaaaaaaaaaaaattggcagactccagagcccacacatttgcactctagactctactgccttcctcatgaagaattttaggacccccgtctggctgtgttgttgcttggggttcaaattctggttgaaagatggcggctgcagtgggaccactattatctctgtcctcacagagttcaagctgaagaagatgtggaaaagtcccaatggaactatccggaacatcctgggggggactgtcttccgggagcccatcatctgcaaaaacatcccacgcctagtccctggctggaccaagcccatcaccattggcaggcacgcccatggcgaccaggtaggccagggtggagaggggatccactgacctgggcaccccccgactggagctcctcgcctagccatcctcttgtctctgcagtacaaggccacagactttgtggcagaccgggccggcactttcaaaatggtcttcaccccaaaagatggcagtggtgtcaaggagtgggaagtgtacaacttccccgcaggcggcgtgggcatgggcatgtacaacaccgacgaggtgaggctggcttgggcatcctgggcccctcctctcccagcttgtctcttcatctctgtctcgtggctttcctttcttctgtaatggcctcattcttaggccacaaagaggcagaaatggccaccagggctgggcatggtggctgacacttgtaatcccggcattttgggaggccaaggcaggattgcttgagaccagaagtttgagaccagcctgggcaacagtgagaccccatctcttgaaacaaaaagaaaagaaaagaaaagaaaagaaatggccacgagcagctccaggcgtactcctgcctgcttggctaccctatcagaagagaatgcctctttatccccagttccagctccaatgccaggctgagtcacatgaccacctctgagccaatcacagaggccctggggagaaaggcttctgattggataagccagggacacactgtcaaccccagcgctaggcctgggaattagccctgccccagccacatgtagtctaaggcggtgctttccaaaaaaaagttgtttccaaaaaaaaaaaaaaaaaggcggtgttttccaaaaaaaaaaaaaaggcggtgttttacaaagcaaagttgagagggagaggctgggccagcagaaacatcgtgtgcactgcacggaggctggtgttaaacagtcgcgtgggcggcggggtaccgttcctggagagctgggccttgccctgggaggtgggaggttgccggcaatcgccaggctagggcaccacgccagggccctgtctctccccctgcagtccatctcaggttttgcgcacagctgcttccagtatgccatccagaagaaatggccgctgtacatgagcaccaagaacaccatactgaaagcctacgatgggcgtttcaaggacatcttccaggagatctttgacaagtaaagcctcatccatgtactctgtggcctttcttcccttccccccatgctgttcccatcctaccctgggaaggtcgctattagagtgcatttggctcagctccgaggctcagggagggatccccaacctgtcagccttctgccctctccccataacagacctttttactcccaggcactataagaccgacttcgacaagaataagatctggtatgagcaccggctcattgatgacatggtggctcaggtcctcaagtcttcgggtggctttgtgtgggcctgcaagaactatgacggagatgtgcagtcagacatcctggcccagggtacgctgggaggacgctgctccctgtgggggtggactgttggtcttctctggctggagggattttcagacccttttggtagaaaactctagtggagcaggactcattgcagggtcctctgtggacagcagggggcaggagctccctggcatctgagtgagagaggatcacaaattaggtttggactggtgtatgactgaggctgctcttagagagaaaggagggcacaggaattccagggctaggttgacagccctaaggttgctccagtccatgatggagcctcatgactctttggttccagtaggaaaatgggatcaagcgggactggaggttctttaaactacagcgaatgcattttgcatagtgaacctaatggatggaagagaatgttggggaagcctgcaaacccccaagccctagggtgtcctaaaagacaaatcagattgcagtaaaaagtataggtgatgaggtcaggagtttgagaccagcgtggccaacatgacgaaaccccgtctctactaaaaatgcaaaaaaatttagctgggcatggaggcgggcacagtcatggacacctgtaatcccagctacttgagaggctgaggcaggagaatctcttgaacccaggaggtggaggttgcagtgagccgagattgcgccattgcactccagcctgggcaacaagggtgaaactccgtctccaaaaaaaataaaaaataaaaaataaagtataggtgagaccattagagaagaacagcgagaaatgggccaattgtgttgggaacaagagcaggaaaggtggtaggtgtgggaaacaggcaggacatagagagtgaaagagaaacctaagcttaccagggcagctcctggtatggggaaaagggaagcccaggacgggttctgatgaaaagcagagtcttgttactttgtgtaaacctttggctttactttggaaagttcagtggggatcctctcaccgaacaattcccatctaggatagaaggatgaaacgccatagcagatgaggcagaggcatgaaaatgaaccttaagtgtctaaggaatggtgtgaaggtgcaggtaaaatggagaagggattgcttacagccaaaattcttagaattcatagacaaagcattttcctgtagatgttgtccatgttttctttcttcaatccttattttggtgtcatgggggtgcctttgaagccctggagtccttgacaatagaaaagcttgctggagttgacatgtggatttgggagcattcagaaattaatcaaaaagcttgaaagtggcctaagagagtagctgcaaaagtgattgttgggaaatagctgaccacagctttattgttttccttgaatgtgcctccgggtcacgagccaggctgggtggcaggctgtgggggaccacaggactctgtggcctcttgggccctgtgggccacaggctgtggggcgcctggagctggtgacacagatggatgttaatgtgctggtgatagccaccctccagtgccctccagccctgtgctgggccctggagacccacaggagggtgaagagacctggaacagtccctgtcctcccagttgcagctgggggaggctgagtagagccacgaactatggcagctacaatattgggttgtagagggcagcagggctcagctgggtggccccaggagaggcgaggccctgagagaaaggctttctaccctccaggctttggctcccttggcctgatgacgtccgtcctggtctgccctgatgggaagacgattgaggctgaggccgctcatgggaccgtcacccgccactatcgggagcaccagaaggtgagtgcagggcatggggcctcacaatgcccctctaccccaggggcctaaagcccactgggctgagggctggaaggacgagcctgacccaccagcccaggggcgggcagctgtatctgggcagcctcctcactagctggctcggctcttgatctccctgcaacccccgtccccagggccggcccaccagcaccaaccccatcgccagcatctttgcctggacacgtggcctggagcaccgggggaagctggatgggaaccaagacctcatcaggtgagcatggagggagaggccggagctgcccaggacaggctctgggtcctgcccttgcacagatggggtctcattctgccccatccccataggtttgcccagatgctggagaaggtgtgcgtggagacggtggagagtggagccatgaccaaggacctggcgggctgcattcacggcctcagcaagtgcgtggcctggggtggtaggcagaccctggggccatgtgggaggaagccaggggacccctcctaaagtccccctcatgagtcagcttagctgcaagggggcctggggcaggacatgtccacagccctgagagactgctcaggccagcctctgtagaccaggtctagtgagttcctccccatccctgaggctggctactcagagctccttccgaccctgcccacacccctttcccagcttctctcaggagagcagttacctggcaagacagaccctgcgggtgcctgccgaatctcggtggcctcatggtagggggctatagctggcctctggcctgtagtctgggtggcaggagatggctgttctgatgcccaagctcaggctgttccctggaagggcctggctgatggccttggcaaggcctggagccacccctgatgtgagtggtgctctctctgcagtgtgaagctgaacgagcacttcctgaacaccacggacttcctcgacaccatcaagagcaacctggacagagccctgggcaggcagtagggggaggcgccacccatggctgcagtggaggggccagggctgagccggcgggtcctcctgagcgcggcagagggtgagcctcacagcccctctctggaggcctttctaggggatgtttttttataagccagatgtttttaaaagcatatgtgtgtttcccctcatggtgacgtgaggcaggagcagtgcgttttacctcagccagtcagtatgttttgcatactgtaatttatattgcccttggaacacatggtgccatatttagctactaaaaagctcttcacaaaattgtctgctgtgtttgtccctgaggggaggaggtagtgggaccctgaggcagaggccctgctagagctggcaggttcccctggggcagaccagagcacctcaggaaggggctgccacggcagggaagggaccaggcagccctgggagcccgcattccacaggggcccactgcggagttctcggacactcagggcacaggcctgtgggttccctggaattttctagcatgatccagtttctgtgtccagttctccattctgagagtcaatcagttcctgataggttgtcattgatttttttcttcgttggttttaaccttctaaacatctccaggccactttcttagcctttttctaggtactaaaaagaggtcctacccacacctgcctcacacttctcctttccaaggctgcctgagtttggaggggcttgggtgtgtgtgaacaagggccctgcattgtctaggcctgcagttcccaggcttgggttcactttcaccatgcattggcaaaactagaaaagtaagcttgtgacaaattgttctcggccgggcacagtggcgcacgcctataatccctgtactttgggaggctgaggtgggtggatcacttgaggccaggagttcgagaccagcctggccaacatggtgaaaccccatctctactcaaaatacaaaaattagccaggcgtggtgatgcgcacctgcagtcccagctactcgggaggctgaggcaggaaaatggcttgaacctgggaggcagaggttgcagtgagccgagactgcaccactgcactccagcctgggtgacagagcaagactctgtctcaaaaaaaaaaaaaaaaaaaaaaaaaaaaaaattgctctatgcaagttttttagccccatgtaaaaaggaaaaataaaagttgctctaaagctacatataatcaaaa

SEQ ID NO: 40 - RNA, 2658 nt, Homo sapiens
  source 1..2658: mol_type = mRNA
agcccgcggccaggcagccgggaggagcggcgcgcgctcggacctctcccgccctgctcgttcgctctccagcttgggatggccggctacctgcgggtcgtgcgctcgctctgcagagcctcaggctcgcggccggcctgggcgccggcggccctgacagcccccacctcgcaagagcagccgcggcgccactatgccgacaaaaggatcaaggtggcgaagcccgtggtggagatggatggtgatgagatgacccgtattatctggcagttcatcaaggagaagctcatcctgccccacgtggacatccagctaaagtattttgacctcgggctcccaaaccgtgaccagactgatgaccaggtcaccattgactctgcactggccacccagaagtacagtgtggctgtcaagtgtgccaccatcacccctgatgaggcccgtgtggaagagttcaagctgaagaagatgtggaaaagtcccaatggaactatccggaacatcctgggggggactgtcttccgggagcccatcatctgcaaaaacatcccacgcctagtccctggctggaccaagcccatcaccattggcaggcacgcccatggcgaccagtacaaggccacagactttgtggcagaccgggccggcactttcaaaatggtcttcaccccaaaagatggcagtggtgtcaaggagtgggaagtgtacaacttccccgcaggcggcgtgggcatgggcatgtacaacaccgacgagtccatctcaggttttgcgcacagctgcttccagtatgccatccagaagaaatggccgctgtacatgagcaccaagaacaccatactgaaagcctacgatgggcgtttcaaggacatcttccaggagatctttgacaagcactataagaccgacttcgacaagaataagatctggtatgagcaccggctcattgatgacatggtggctcaggtcctcaagtcttcgggtggctttgtgtgggcctgcaagaactatgacggagatgtgcagtcagacatcctggcccagggctttggctcccttggcctgatgacgtccgtcctggtctgccctgatgggaagacgattgaggctgaggccgctcatgggaccgtcacccgccactatcgggagcaccagaagggccggcccaccagcaccaaccccatcgccagcatctttgcctggacacgtggcctggagcaccgggggaagctggatgggaaccaagacctcatcaggtttgcccagatgctggagaaggtgtgcgtggagacggtggagagtggagccatgaccaaggacctggcgggctgcattcacggcctcagcaatgtgaagctgaacgagcacttcctgaacaccacggacttcctcgacaccatcaagagcaacctggacagagccctgggcaggcagtagggggaggcgccacccatggctgcagtggaggggccagggctgagccggcgggtcctcctgagcgcggcagagggtgagcctcacagcccctctctggaggcctttctaggggatgtttttttataagccagatgtttttaaaagcatatgtgtgtttcccctcatggtgacgtgaggcaggagcagtgcgttttacctcagccagtcagtatgttttgcatactgtaatttatattgcccttggaacacatggtgccatatttagctactaaaaagctcttcacaaaattgtctgctgtgtttgtccctgaggggaggaggtagtgggaccctgaggcagaggccctgctagagctggcaggttcccctggggcagaccagagcacctcaggaaggggctgccacggcagggaagggaccaggcagccctgggagcccgcattccacaggggcccactgcggagttctcggacactcagggcacaggcctgtgggttccctggaattttctagcatgatccagtttctgtgtccagttctccattctgagagtcaatcagttcctgataggttgtcattgatttttttcttcgttggttttaaccttctaaacatctccaggccactttcttagcctttttctaggtactaaaaagaggtcctacccacacctgcctcacacttctcctttccaaggctgcctgagtttggaggggcttgggtgtgtgtgaacaagggccctgcattgtctaggcctgcagttcccaggcttgggttcactttcaccatgcattggcaaaactagaaaagtaagcttgtgacaaattgttctcggccgggcacagtggcgcacgcctataatccctgtactttgggaggctgaggtgggtggatcacttgaggccaggagttcgagaccagcctggccaacatggtgaaaccccatctctactcaaaatacaaaaattagccaggcgtggtgatgcgcacctgcagtcccagctactcgggaggctgaggcaggaaaatggcttgaacctgggaggcagaggttgcagtgagccgagactgcaccactgcactccagcctgggtgacagagcaagactctgtctcaaaaaaaaaaaaaaaaaaaaaaaaaaaaaaattgctctatgcaagttttttagccccatgtaaaaaggaaaaataaaagttgctctaaagctacatataatcaaaa

SEQ ID NO: 41 - RNA, 1578 nt, Homo sapiens
  source 1..1578: mol_type = mRNA
attttgcaacgccataggcttccagcgactgctggtgatgtttctgatgccgacaaaaggatcaaggtggcgaagcccgtggtggagatggatggtgatgagatgacccgtattatctggcagttcatcaaggagaagctcatcctgccccacgtggacatccagctaaagtattttgacctcgggctcccaaaccgtgaccagactgatgaccaggtcaccattgactctgcactggccacccagaagtacagtgtggctgtcaagtgtgccaccatcacccctgatgaggcccgtgtggaagagttcaagctgaagaagatgtggaaaagtcccaatggaactatccggaacatcctgggggggactgtcttccgggagcccatcatctgcaaaaacatcccacgcctagtccctggctggaccaagcccatcaccattggcaggcacgcccatggcgaccagtacaaggccacagactttgtggcagaccgggccggcactttcaaaatggtcttcaccccaaaagatggcagtggtgtcaaggagtgggaagtgtacaacttccccgcaggcggcgtgggcatgggcatgtacaacaccgacgagtccatctcaggttttgcgcacagctgcttccagtatgccatccagaagaaatggccgctgtacatgagcaccaagaacaccatactgaaagcctacgatgggcgtttcaaggacatcttccaggagatctttgacaagcactataagaccgacttcgacaagaataagatctggtatgagcaccggctcattgatgacatggtggctcaggtcctcaagtcttcgggtggctttgtgtgggcctgcaagaactatgacggagatgtgcagtcagacatcctggcccagggctttggctcccttggcctgatgacgtccgtcctggtctgccctgatgggaagacgattgaggctgaggccgctcatgggaccgtcacccgccactatcgggagcaccagaagggccggcccaccagcaccaaccccatcgccagcatctttgcctggacacgtggcctggagcaccgggggaagctggatgggaaccaagacctcatcaggtttgcccagatgctggagaaggtgtgcgtggagacggtggagagtggagccatgaccaaggacctggcgggctgcattcacggcctcagcaatgtgaagctgaacgagcacttcctgaacaccacggacttcctcgacaccatcaagagcaacctggacagagccctgggcaggcagtagggggaggcgccacccatggctgcagtggaggggccagggctgagccggcgggtcctcctgagcgcggcagagggtgagcctcacagcccctctctggaggcctttctaggggatgtttttttataagccagatgtttttaaaagcatatgtgtgtttcccctcatggtgacgtgaggcaggagcagtgcgttttacctcagccagtcagtatgttttgcatactgtaatttatattgcccttggaacacatggtgccatatttagctactaaaaagctcttcacaaaa

SEQ ID NO: 42 - RNA, 2400 nt, Homo sapiens
  source 1..2400: mol_type = mRNA
agcccgcggccaggcagccgggaggagcggcgcgcgctcggacctctcccgccctgctcgttcgctctccagcttgggatggccggctacctgcgggtcgtgcgctcgctctgcagagcctcaggctcgcggccggcctgggcgccggcggccctgacagcccccacctcgcaagagcagccgcggcgccactagttcaagctgaagaagatgtggaaaagtcccaatggaactatccggaacatcctgggggggactgtcttccgggagcccatcatctgcaaaaacatcccacgcctagtccctggctggaccaagcccatcaccattggcaggcacgcccatggcgaccagtacaaggccacagactttgtggcagaccgggccggcactttcaaaatggtcttcaccccaaaagatggcagtggtgtcaaggagtgggaagtgtacaacttccccgcaggcggcgtgggcatgggcatgtacaacaccgacgagtccatctcaggttttgcgcacagctgcttccagtatgccatccagaagaaatggccgctgtacatgagcaccaagaacaccatactgaaagcctacgatgggcgtttcaaggacatcttccaggagatctttgacaagcactataagaccgacttcgacaagaataagatctggtatgagcaccggctcattgatgacatggtggctcaggtcctcaagtcttcgggtggctttgtgtgggcctgcaagaactatgacggagatgtgcagtcagacatcctggcccagggctttggctcccttggcctgatgacgtccgtcctggtctgccctgatgggaagacgattgaggctgaggccgctcatgggaccgtcacccgccactatcgggagcaccagaagggccggcccaccagcaccaaccccatcgccagcatctttgcctggacacgtggcctggagcaccgggggaagctggatgggaaccaagacctcatcaggtttgcccagatgctggagaaggtgtgcgtggagacggtggagagtggagccatgaccaaggacctggcgggctgcattcacggcctcagcaatgtgaagctgaacgagcacttcctgaacaccacggacttcctcgacaccatcaagagcaacctggacagagccctgggcaggcagtagggggaggcgccacccatggctgcagtggaggggccagggctgagccggcgggtcctcctgagcgcggcagagggtgagcctcacagcccctctctggaggcctttctaggggatgtttttttataagccagatgtttttaaaagcatatgtgtgtttcccctcatggtgacgtgaggcaggagcagtgcgttttacctcagccagtcagtatgttttgcatactgtaatttatattgcccttggaacacatggtgccatatttagctactaaaaagctcttcacaaaattgtctgctgtgtttgtccctgaggggaggaggtagtgggaccctgaggcagaggccctgctagagctggcaggttcccctggggcagaccagagcacctcaggaaggggctgccacggcagggaagggaccaggcagccctgggagcccgcattccacaggggcccactgcggagttctcggacactcagggcacaggcctgtgggttccctggaattttctagcatgatccagtttctgtgtccagttctccattctgagagtcaatcagttcctgataggttgtcattgatttttttcttcgttggttttaaccttctaaacatctccaggccactttcttagcctttttctaggtactaaaaagaggtcctacccacacctgcctcacacttctcctttccaaggctgcctgagtttggaggggcttgggtgtgtgtgaacaagggccctgcattgtctaggcctgcagttcccaggcttgggttcactttcaccatgcattggcaaaactagaaaagtaagcttgtgacaaattgttctcggccgggcacagtggcgcacgcctataatccctgtactttgggaggctgaggtgggtggatcacttgaggccaggagttcgagaccagcctggccaacatggtgaaaccccatctctactcaaaatacaaaaattagccaggcgtggtgatgcgcacctgcagtcccagctactcgggaggctgaggcaggaaaatggcttgaacctgggaggcagaggttgcagtgagccgagactgcaccactgcactccagcctgggtgacagagcaagactctgtctcaaaaaaaaaaaaaaaaaaaaaaaaaaaaaaattgctctatgcaagttttttagccccatgtaaaaaggaaaaataaaagttgctctaaagctacatataatcaaaa

(19) United States
(12) Patent Application Publication
(10) Pub. No.: US 2025/0188169 A1
(43) Pub. Date: Jun. 12, 2025

(54) METHOD FOR TREATING CLONAL HEMATOPOIETIC BLOOD DISORDERS

(71) Applicant: THE CHILDREN'S MEDICAL CENTER CORPORATION, Boston, MA (US)

(72) Inventors: Leonard I. ZON, Brookline, MA (US); Jonathan E. HENNINGER, Sommerville, MA (US); Serine AVAGYAN, San Francisco, CA (US)

(73) Assignee: THE CHILDREN'S MEDICAL CENTER CORPORATION, Boston, MA (US)

(21) Appl. No.: 18/843,935

(22) PCT Filed: Mar. 31, 2023
(86) PCT No.: PCT/US2023/065185

§ 371 Date: Sep. 4, 2024

Related U.S. Application Data
(60) Provisional application No. 63/326,596, filed on Apr. 1, 2022.

(51) Int. Cl. C07K 16/28; A61K 31/404; A61K 31/415; A61K 31/4745; C12N 15/113

(52) U.S. Cl. CPC C07K 16/2857; A61K 31/404; A61K 31/415; A61K 31/4745; C12N 15/1138; C07K 2317/24; C12N 2310/11; C12N 2310/12; C12N 2310/16

(57) ABSTRACT

The disclosure provides method for treating clonal hematopoiesis disorders such as clonal hematopoiesis, myelodysplasia, and leukemia.

### CROSS REFERENCE TO RELATED APPLICATIONS

This application is a 35 U.S.C. § 371 National Phase Entry Application of International Application No. PCT/US2023/065185 filed Mar. 31, 2023, which claims benefit under 35 U.S.C. § 119(e) of U.S. Provisional Application No. 63/326,596, filed Apr. 1, 2022, the contents of both of which are incorporated herein by reference in their entireties.

### GOVERNMENT SUPPORT

This invention was made with government support under grant numbers HL144780, HL131477 and HL126338, awarded by the National Institutes of Health. The government has certain rights in the invention.

### SEQUENCE LISTING

The instant application contains a Sequence Listing that has been submitted in XML format via Patent Center and is hereby incorporated by reference in its entirety. Said XML copy, created on Mar. 9, 2023 is named 701039-191350WOPT_SL.xml and is 455,674 bytes in size.

### TECHNICAL FIELD

The technology described herein relates to methods of treating clonal hematopoiesis disorders such as clonal hematopoiesis, myelodysplastic syndromes, and leukemia.

### BACKGROUND

Clonal hematopoiesis (CH) is a state of clonal dominance of a mutant hematopoietic stem and progenitor cell (HSPC) promoted by unknown mechanisms. Individuals with CH have increased levels of inflammatory cytokines in blood. Mutations enabling competitive outgrowth may be due to aging, selective resistance to chemotherapy, or founder effects in HSPC transplantation. There is a need for methods and compositions for treating clonal hematopoietic blood disorders.

### SUMMARY

As described herein, the inventors have identified a gene that is important in the pathogenesis of CH. CH is established by introduction of mutations in certain genes, including ASML1, DNMT3A, IDH1, IDH2, and others. This state is considered a preleukemia with a high risk of progressing to myelodysplastic syndromes or leukemia over time. The inventors have discovered inter alia that the mutant blood stem cells in clonal hematopoiesis overexpress the gene NR4A1 which promotes the increased fitness and survival of the mutant stem cells compared to wildtype stem cells. When the inventors mutated both copies of NR4A1 in stem cells that were mutant for ASXL1, the competitive advantage was abrogated. Accordingly, in one aspect, provided herein is method for treating a clonal hematopoietic disease or disorder in a subject in need of treatment. Generally, the method comprises inhibiting a member of the nuclear receptor 4A (NR4A) family. For example, by administering an effective amount of an inhibitor of a member of the NR4A family to the subject.

In some embodiments of any one of the aspects described herein, the clonal hematopoietic disease or disorder is clonal hematopoiesis, myelodysplastic syndromes (MDS) or leukemia.

In some embodiments, the clonal hematopoietic disease or disorder is leukemia. For example, clonal hematopoietic disease or disorder is acute myelogenous leukemia (AML) or chronic myeloid leukemia (CML).

In some embodiment, the clonal hematopoietic disease or disorder is myelodysplastic syndromes. Exemplary myelodysplastic syndromes include, but are not limited to, MDS with multilineage dysplasia (MDS-MLD), MDS with single lineage dysplasia (MDS-SLD), MDA with ring sideroblasts (MDS-RS), MDS with excess blasts (MDS-EB), MDS with isolated del(5q), and/or MDS unclassifiable (MDS-U).

In another aspect provided herein is a method for inhibiting clonal expansion of hematopoietic stem and progenitor cells. The method comprises administering an effective amount of an inhibitor of a member of the NR4A family to a HSPC. It is noted that administering to the cell can be in vitro or in vivo. For example, when the administering to the cell is in vivo, the compound can be administered to a subject. The subject can be one having a clonal hematopoietic disease or disorder or in need of treatment for clonal hematopoietic disease or disorder.

In some embodiments of any one of the aspects described herein, the NR4A family member is selected from the group consisting of NR4A1, NR4A2 and NR4A3. In some preferred embodiments, the NR4A family member is NR4A1.

In some embodiments of any one of the aspects described herein, the inhibitor binds with the NR4A family member or with a nucleic acid encoding the NR4A family member.

Exemplary inhibitors that can be used include, but are not limited to, nucleic acids, antibodies, and small molecules. Accordingly, in some embodiments of any one of the aspects described herein, the inhibitor is a nucleic acid. For example, the inhibitor is a nucleic acid selected from the group consisting of siRNAs, antisense oligonucleotides, aptamers, ribozymes, and triplex forming oligonucleotides. Generally, the nucleic acid inhibitor comprises a nucleotide sequence substantially complementary to at least a portion of a nucleic acid encoding the NR4A family member. For example, the inhibitor comprises a nucleotide sequence substantially complementary to at least 15 contiguous nucleotides of a nucleic acid encoding the NR4A family member.

In some embodiments of any one of the aspects described herein, inhibitor is an antibody or an antigen binding fragment thereof. For example, the inhibitor is an antibody or an antigen binding fragment thereof that binds the NR4A family member. Generally, the antibody or the antigen binding fragment thereof binds an epitope on the NR4A family member such that the binding inhibits a function and/or activity of the NR4A family member. It is noted that the antibody can be a polyclonal or monoclonal antibody. Accordingly, in some embodiments of any one of the aspects described herein, the inhibitor is a monoclonal antibody. In some other embodiments of any one of the aspects described herein, the inhibitor is a polyclonal antibody. The antibody can be a chimeric antibody. In some embodiments of any one of the aspects described herein, the antibody is a human antibody or a humanized antibody.

In some embodiments of any one of the aspects described herein, the inhibitor is a monoclonal human antibody or monoclonal humanized antibody.

Small molecule inhibitors of NR4A family members are also known in the art. Accordingly, in some embodiments of any one of the aspects described herein, the inhibitor is a small molecule. Generally, the small molecule inhibitor binds with the NR4A family member and inhibits a function and/or activity of the NR4A family member. Exemplary small molecule inhibitors include, but are not limited to 1,1-bis(3′-indolyl)-1-(p-hydroxyphenyl)methane (DIM-C-pPhOH), 1,1-bis(3′-indolyl)-1-(3-chloro-4-hydroxy-5-methoxyphenyl)methane (DIM-C-pPhOh-3-Cl-5-OCH₃), 1,1-bis(3′-indolyl)-1-(3,5-dibromo-4-hydroxyphenyl)methane (DIM-C-pPhOH-3,5-Br2), camptothecin (CPT), a cyclooxygenase (COX)-2 inhibitor (celecoxib analogue SC-236), Erlotinib, Afatinib, Bosutinib, Dasatinib, and KU171309.

The subject undergoing treatment can also be given additional therapies. For example, the described herein further comprises co-administering one or more additional therapeutic agents to the subject. It is noted that the additional therapeutic agent can be administered prior to, simultaneously with or after administering the inhibitor of the NR4A family member. Exemplary additional therapeutic agents that can be co-administered with the inhibitor of the NR4A family member include, but are not limited to immunomodulatory agents and anti-inflammatory agents. Accordingly, in some embodiments of any one of the aspects described herein, the inhibitor of the NR4A family member include, but method further comprises co-administering an immunomodulatory agent to the subject. In some embodiments of any one of the aspects described herein, the method further comprises co-administering an anti-inflammatory agent to the subject.

The inventors have discovered inter alia that when the expression of NR4A1 is inhibited in HSPC cells that are mutant for certain genes the increased fitness and survival of the mutant stem cells compared to wildtype stem cells is abrogated. Accordingly, in some embodiments of any one of the aspects described herein, the subject has at least one mutation in a nucleic acid encoding ASXL transcriptional regulator 1 (ASXL1), DNA (cytosine-5)-methyltransferase 3A (DNMT3A), isocitrate dehydrogenase (NADP(+)) 1(IDH1) or isocitrate dehydrogenase (NADP(+)) 2 (IDH 2). The method described herein can include a step of identifying and/or selecting a subject with at least one mutation in a nucleic acid encoding ASXL1, DNMT3A, IDH1 or IDH 2 prior to onset of the treatment regime. For example, the method can further comprise a step of assaying a sample from the subject for at least one mutation in a nucleic acid encoding ASXL1, DNMT3A, IDH1 or IDH 2 prior to onset of the treatment regime.

In some embodiments, the method further comprises a step of assaying a HSPC from the subject for at least one mutation in a nucleic acid encoding ASXL1, DNMT3A, IDH1 or IDH2 prior to onset of the treatment regime.

### BRIEF DESCRIPTION OF THE DRAWINGS

FIG. 1 shows early genetic targeting nr4a1 during development prevented clonal growth of mutant cells. Pharmacologic inhibition of nr4a1 can halt or reduce mutant clonal growth over time.

FIG. 2 shows the experimental set up to target the pathways involved in clonal hematopoiesis. Zebrafish were generated with mosaic mutations in asxl1 using the TWISTR method (Avagyan et a1 Science 2021, PMID 34735227). Briefly, they were injected with guide-RNAs targeting asxl1 gene with Cas9 mRNA into 1-cell embryos of zebrafish, and grown to adulthood. At 3 months retroortibital bleeding was performed on the adult zebrafish and measure the size of the mutant clones in the peripheral blood by sequencing the genomic DNA at the targeting locus of asxl1. Zebrafish with at least 1 mutant allele of 5% of greater allele size were chosen for the cohorts, randomly assigned to be treated with either DMSO or NR4A1 inhibitor (DIM-C). The dose of DIM-C was determined in a previously conducted pilot experiment. Zebrafish with mosaic mutations in asxl1 are treated with either DMSO or an NR4A1 inhibitor (DIM-C) and analyzed over a three-month period. Peripheral blood was collected every month to determine the size of the mutant clones over time. No physical abnormalities were observed in the treatment zebrafish over this period of time. RO=retro-orbital bleed, RO1=first RO, pre-treatment, RO2 through RO4=monthly RO bleeds following treatment.

FIG. 3 shows the asxl1-mutant clonal dynamics in individual zebrafish after DMSO treatment over a three-month period. Overall, there is an increase in the size of the mutant clones that harbor frameshift indels over a three-month period. Conversely, mutant clones with non-frameshift mutations did not change (fish 5) or diminished (fish 4 and 11) over the same period of time. VAF=variant allele fraction, indel=insertion/deletion CRISPR edit.

FIG. 4 depicts the dynamics of asxl1-mutant clones with frameshift and non-frameshift indels after DMSO treatment in all zebrafish (n=18) in the control cohort in a three-month period. Note that zebrafish had on average of 5.2 frameshift and 2.1 non-frameshift indels per fish, with total of 93 frameshift and 37 non-frameshift indels shown. Overall, there is more mutant clonal expansion resulting from frameshift indels than from non-frameshift indels with DMSO treatment, suggesting a positive selection of frameshift mutant clones.

FIG. 5 shows the clonal dynamics of asxl1-mutant clones in individual zebrafish after an NR4A1 inhibitor (DIM-C) treatment over a three-month period. Unlike in zebrafish treatment with DMSO, the expansion of asxl1-mutant clones with frameshift indels in zebrafish treated with DIM-C, the NR4A1 inhibitor, is less pronounced and plateaus by 3 months. Mutant clones with non-frameshift indels are similarly unchanged over time.

FIG. 6 shows the dynamics of asxl1-mutant clones harboring frameshift and non-frameshift indels with an NR4A1 inhibitor (DIM-C) treatment in all zebrafish (n=12) in a three-month period. The zebrafish harbored more than 1 mutant asxl1 clone, on average 5.6 frameshift and 1.6 non-frameshift clones, not significantly different from the DMSO control cohort. A total of 73 frameshift and 21 non-frameshift clones were quantified. Overall, there is significantly abated asxl1-mutant clonal expansion in zebrafish treated with DIM-C.

FIG. 7 shows the dynamics of asxl1-mutant clones harboring frameshift indels in zebrafish treatment with either DMSO or an NR4A1 inhibitor (DIM-C) for three months. There is less expansion of asxl1-mutant clones in zebrafish that were treated with DIM-C as compared to those treated with DMSO. Clones that had a VAF of 5% or greater in at least 1 time point and were present in at least 2 time points were graphed in FIG. 8.

FIG. 8 depicts a schematic where ASXL1 mutant frameshift indel zebrafish receive DMSO or an NR4A1 inhibitor (DIM-C) treatment over a three-month period (age of zebrafish is rounded to 3 or 7 months, respectively). The change in clonal size of the mutant clones over a three-month period is shown. The change is calculated at percent change of the clone size (VAF at RO4 compared to VAF at RO1) compared to the pretreatment clone size (VAF at RO1). After treatment of DIM-C, asxl1-mutant clones with frameshift-indels had significantly less expansion as compared to those after DMSO treatment, demonstrating the effect of DIM-C in preventing the expansion of ASXL1 mutant clones. The figure also shows the analysis of the data using the Wilcoxon rank test which measures the difference of the clonal size change compared to 0, the null hypothesis that the change in clone size is absent. With this test as well, we observe significant positive change in clonal expansion of frameshift mutant clones in zebrafish treated with DMSO, and no significant change in the asxl1 frameshift indel containing clone size in NR4A1 inhibitor DIM-C treated zebrafish. In both cohorts, non-frameshift clones did not change significantly over time, consistent with our hypothesis that only frameshift clones are functionally consequential.

FIG. 9 shows how targeting pathways involved in driving clonal hematopoiesis.

FIG. 10 depicts the experimental set up, which examines the timing of treatment and retro-orbital bleeding for peripheral blood cell genomic DNA.

FIG. 11 examines the treatment of NR4A1 inhibitor DIMC.

FIG. 12 shows zebrabow clonality analysis in marrow myeloid cells.

FIGS. 13A-13H depicts clonal dynamic patterns in DMSO treatment group.

FIGS. 14A-14F depicts clonal dynamic patters in DIMC treatment group.

FIG. 15 compares the analysis of clone size prior to NR4A1 inhibitor treatment.

FIG. 16 examines the analysis of clone size changes with NR4A1 inhibitor treatment.

FIGS. 17A-17B shows the change in clone size based on the pre-treatment clone size of either DMSO (FIG. 17A) or DIMC (FIG. 17B).

FIGS. 18A-18D examines the change in clone size of all clones present at pre-treatment and end of treatment retro-orbital bleeding for frameshift clones after DMSO (FIG. 18A) and DIMC (FIG. 18B) and non-frameshift clones after DMSO (FIG. 18C) and DIMC (FIG. 18D) treatment.

FIGS. 19A-19D shows that the greatest effect is on large clones with frameshift asxl1 edits frameshift clones after DMSO (FIG. 19A) and DIMC (FIG. 19B) and non-frameshift clones after DMSO (FIG. 19C) and DIMC (FIG. 19D) treatment.

### DETAILED DESCRIPTION

The various aspects described herein are based in part on the inventors' discovery that mutant blood stem cells in clonal hematopoiesis overexpress the gene NR4A1 which promotes the increased fitness and survival of the mutant stem cells compared to wildtype stem cells. Inhibiting the NR4A1 or expression of a nucleic acid encoding the NR4A1 nullifies this advantage. Accordingly, in one aspect, provided herein is method for treating a clonal hematopoietic disease or disorder in a subject in need of treatment by inhibiting NR4A1 directly or by inhibiting the expression of a nucleic acid encoding NR4A1 in a HSPC.

The nuclear receptor subfamily 4A (NR4A) is a family of orphan nuclear receptors which act as transcription factors in neuron development and maintenance. The NR4A family is composed of three members—NR4A1 (Nur77/TR3/NGFI-B), NR4A2 (Nurr1/TINUR/NOT) and NR4A3 (MINOR/CSMF). While currently defined as ligandless, these transcription factors have been shown to regulate varied processes across a host of tissues. Of particular interest, the NR4A family impinge, in a tissue dependent fashion, on cellular proliferation, apoptosis and fuel utilization. The regulation of these processes occurs through both nuclear and non-genomic pathways.

The three members have a high degree of sequence homology, with each containing a ligand-independent activation function-1 domain, transactivation domain necessary for transcriptional activity and cofactor binding, a DNA binding domain and a ligand binding domain containing a ligand-dependent AF-2 transactivation domain. While currently defined as orphaned receptors with no known endogenous ligand, there have been suggestions that the NR4A family's transcription factor function may be regulated through binding unsaturated fatty acids in the ligand binding domain.

The NR4A family binds directly as monomers or homodimers to promoters of target genes that contain the NBRE (NGFIB Response Element-AAAGGTCA) motif. The NR4As can also form heterodimers and bind to the NuRE (Nur Response element-AAT(G/A)(C/T)CA). Family members NR4A1 and NR4A2 have been shown to forms dimers with retinoid X receptors and bind to the DR5 elements. While there is a high degree of similarity between the three family members, the different members have differing affinity for co-factors and response elements, thus giving specificity to each.

The NR4A family is widely expressed across various tissues. NR4A family members have been shown to be critical in the hematopoietic system, adipose tissue, liver, muscle and β-cells, among other tissues. In these tissues, the function of Nr4a family members fall into one of two categories. The majority of NR4A activity is due to direct activation or repression of transcriptional target expression. However, a growing body of information is demonstrating a direct non-genomic role of NR4A family members through interaction with binding partners.

The NR4A family members are known to regulate cellular proliferation in a tissue dependent manner. Key genes shown to be regulated by NR4A family members include cyclins, cyclin dependent kinases and other ancillary cell cycle genes. Due to this, NR4A1, NR4A2 and NR4A3 are potential therapeutic targets in many cancers, however their specific roles vary between tissues and among tumors from the same organ.

The NR4A family members referred to in this aspect, and all aspects and embodiments described herein in this application, comprises the nucleotide sequences of NR4A1, NR4A2, and NR4A3 with NCBI nucleotide sequence IDs:

NM_002135.5 (SEQ ID NO: 2, NR4A1 isoform 1 mRNA), NM_173157.3 (SEQ ID NO: 3, NR4A1 isoform 1b mRNA), NM_001202233.2 (SEQ ID NO: 4, NR4A1 isoform 2 mRNA), NM_001202234.2 (SEQ ID NO: 5, NR4A1 isoform 3 mRNA), NM_006186.4 (SEQ ID NO: 7, NR4A2 isoform a mRNA), NM_173173.3 (SEQ ID NO: 8, NR4A2 isoform d mRNA), NC_000009.12 (SEQ ID NO: 9, NR4A3 genomic sequence), NM_006981.4 (SEQ ID NO: 10, NR4A3 isoform a mRNA), NM_173200.3 (SEQ ID NO: 11, NR4A3 isoform b mRNA) and NM_173199.4 (SEQ ID NO: 12, NR4A3 isoform c mRNA) and the amino acid sequences of NR4A1, NR4A2, and NR4A3 with NCBI amino acid sequence IDs: NP_001189162.1 (SEQ ID NO: 13, NR4A1 isoform a), NP_775292.1 (SEQ ID NO: 14, NR4A1 isoform b), NP_002126.2 (SEQ ID NO: 15, NR4A1 isoform 1), NP_775180.1 (SEQ ID NO: 16, NR4A1 isoform 1b), NP_001189163.1 (SEQ ID NO: 17, NR4A1 isoform 3), NP_006177.1 (SEQ ID NO: 18, NR4A2 isoform a), NP_775265.1 (SEQ ID NO: 19, NR4A2 isoform d), NP_008912.2 (SEQ ID NO: 20, NR4A3 isoform a), NP_775292.1 (SEQ ID NO: 21, NR4A3 isoform b), and NP_775291.1 (SEQ ID NO: 22, NR4A3 isoform c).

In some preferred embodiments of any one of the aspects described herein, the NR4A family member is nuclear receptor subfamily 4 group A member 1 (NR4A1), which is encoded on human chromosome 12. NR4A1, also referred to as NUR77 in the art, is a nuclear transcription factor induced by phytohemagglutinin in human lymphocytes and by serum stimulation of arrested fibroblasts. The encoded protein is known to respond to diverse cellular stresses to regulate apoptosis, inflammation, and cell cycle mediation. Mutations in this gene are associated with a variety of human diseases such as pseudohypoaldosteronism and adrenal cortical carcinoma. In some embodiments of any one of the aspects described herein, the inhibitor of a NR4A family member inhibits NR4A1.

In some embodiments of any one of the aspects described herein, the NR4A family member is nuclear receptor subfamily 4 group A member 2 (NR4A2), which is encoded on human chromosome 2. This gene encodes a member of the steroid-thyroid hormone-retinoid receptor superfamily. The encoded protein may act as a transcription factor. Mutations in this gene have been associated with disorders related to dopaminergic dysfunction, including Parkinson disease, schizophrenia, and manic depression. Misregulation of this gene may be associated with rheumatoid arthritis. Alternatively, spliced transcript variants have been described, but their biological validity has not been determined. In some embodiments of any one of the aspects described herein, the inhibitor of a NR4A family member inhibits NR4A2.

In some embodiments of any one of the aspects described herein, the NR4A family member is nuclear receptor subfamily 4 group A member 3 (NR4A3), which is encoded on human chromosome 9. This gene encodes a member of the steroid-thyroid hormone-retinoid receptor superfamily. The encoded protein may act as a transcriptional activator. The protein can efficiently bind the NGFI-B Response Element (NBRE). Three different versions of extraskeletal myxoid chondrosarcomas (EMCs) are the result of reciprocal translocations between this gene and other genes. The translocation breakpoints are associated with NR4A3 (on chromosome 9) and either Ewing Sarcome Breakpoint Region 1 (on chromosome 22), RNA Polymerase II, TATA Box-Binding Protein-Associated Factor, 68-KD (on chromosome 17), or Transcription factor 12 (on chromosome 15). In some embodiments of any one of the aspects described herein, the inhibitor of a NR4A family member inhibits NR4A3.

Embodiments of the various aspects described herein include administering an inhibitor of a NR4A family member to a subject. The inhibitor can directly bind with the NR4A family member or with a nucleic acid encoding the NR4A family member. Exemplary inhibitors that can be used include, but are not limited to, nucleic acids, antibodies, and small molecules.

### Nucleic Acid Inhibitors of NR4A Family

In some embodiments of any one of the aspects described herein, the inhibitor of the NR4A family member is an inhibitory nucleic acid. As used herein, the term “inhibitory nucleic acid” or “nucleic acid inhibitor” refers to a nucleic acid molecule which can inhibit the expression of a nucleic acid, e.g., mRNA encoding the NR4A family member. Exemplary, inhibitory nucleic acids include, but are not limited to, double-stranded RNAs (dsRNAs), inhibitory RNAs (iRNAs), antisense oligonucleotides, aptamers, and the like. In some embodiments of any of the aspects, the inhibitory nucleic acid can be a silencing RNA (siRNA), microRNA (miRNA), or short hairpin RNA (shRNA). Inhibitory nucleic acids can also include guide sequence molecules (e.g., a guide RNA) that function, e.g., in combination with an enzyme, to induce insertions, deletions, indels, and/or mutations of a target, thereby inhibiting the expression of the target.

Generally, a nucleic acid inhibitor comprises a nucleotide sequence that is substantially complementary to at least a portion of a nucleic acid encoding a NR4A family member. For example, the nucleic acid inhibitor comprises a nucleotide sequence that is substantially complementary to at least a portion of a nucleotide sequence selected from SEQ ID NOs: 1-12. In some embodiments, the nucleic acid inhibitor comprises a nucleotide sequence that is substantially complementary to at least a portion of a nucleotide sequence selected from SEQ ID NOs: 2-5, 7-8 and 10-12.

In some embodiments of any of the aspects, the nucleic acid inhibitor comprises a sequence at least 15 nucleotides in length that is substantially complementary to at least a portion of a nucleotide sequence selected from SEQ ID NOs: 1-12. For example, the nucleic acid inhibitor comprises a sequence 15, 16, 17, 18, 19, 20, 21, 22, 23, 24, or 25 nucleotides in length that is substantially complementary to at least a portion of a nucleotide sequence selected from SEQ ID NOs: 2-5, 7-8 and 10-12.

In some embodiments, the nucleic acid inhibitor inhibits NR4A1. For example, the nucleic acid inhibitor comprises a sequence 15, 16, 17, 18, 19, 20, 21, 22, 23, 24, or 25 nucleotides in length that is substantially complementary to at least a portion of a nucleotide sequence selected from SEQ ID NOs: 2-5.

In some embodiments, the nucleic acid inhibitor inhibits NR4A2. For example, the nucleic acid inhibitor comprises a sequence 15, 16, 17, 18, 19, 20, 21, 22, 23, 24, or 25 nucleotides in length that is substantially complementary to at least a portion of a nucleotide sequence selected from SEQ ID NOs: 7 and 8.

In some embodiments, the nucleic acid inhibitor inhibits NR4A3. For example, the nucleic acid inhibitor comprises a sequence 15, 16, 17, 18, 19, 20, 21, 22, 23, 24, or 25 nucleotides in length that is substantially complementary to at least a portion of a nucleotide sequence selected from SEQ ID NOs: 10-12.

Double-stranded RNA molecules (dsRNA) have been shown to block gene expression in a highly conserved regulatory mechanism known as RNA interference (RNAi). A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The other strand (the sense strand) includes a region that is complementary to the antisense strand, such that the two strands hybridize and form a duplex structure when combined under suitable conditions. Generally, the duplex structure is between 15 and 30 base pairs in length inclusive, more generally between 18 and 25 base pairs in length inclusive, yet more generally between 19 and 24 base pairs in length inclusive, and most generally between 19 and 21 base pairs in length, inclusive. Similarly, the region of complementarity to the target sequence is between 15 and 30 base pairs in length inclusive, more generally between 18 and 25 base pairs in length inclusive, yet more generally between 19 and 24 base pairs in length inclusive, and most generally between 19 and 21 base pairs in length nucleotides in length, inclusive. In some embodiments of any of the aspects, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

Accordingly, in some embodiments of any one of the aspects described herein, the nucleic acid inhibitor is a dsRNA, where one strand (e.g., antisense strand) of the dsRNA comprises a nucleotide sequence substantially complementary to a portion, e.g., 15, 16, 17, 18, 19, 20, 21, 22, 23, 24 or 25 contiguous nucleotides of a nucleotide sequence selected from SEQ ID NOs: 1-12. For example, the nucleic acid inhibitor is a dsRNA, where the antisense strand of the dsRNA comprises a nucleotide sequence substantially complementary to a portion, e.g., 15, 16, 17, 18, 19, 20, 21, 22, 23, 24 or 25 contiguous nucleotides of a nucleotide sequence selected from SEQ ID NOs: 2-5. In another non-limiting example, the nucleic acid inhibitor is a dsRNA, where the antisense strand of the dsRNA comprises a nucleotide sequence substantially complementary to a portion, e.g., 15, 16, 17, 18, 19, 20, 21, 22, 23, 24 or 25 contiguous nucleotides of a nucleotide sequence selected from SEQ ID NOs: 7-8. In yet another non-limiting example, the nucleic acid inhibitor is a dsRNA, where the antisense strand of the dsRNA comprises a nucleotide sequence substantially complementary to a portion, e.g., 15, 16, 17, 18, 19, 20, 21, 22, 23, 24 or 25 contiguous nucleotides of a nucleotide sequence selected from SEQ ID NOs: 10-12.

Based on the knowledge in the art, one skilled in the art would be able to design further siRNA, shRNA, or miRNA to target the nucleic acid sequence of any one of SEQ ID NOs: 2-6, 7-8 or 10-12. Double-stranded RNAs useful for targeting expression of a NR4A family member enzyme can be readily designed and tested. Chalk et al. (Nucl. Acids Res. 33: D131-D134 (2005)) describe a database of siRNA sequences and a predictor of siRNA sequences. Linked to the sequences in the database is information such as siRNA thermodynamic properties and the potential for sequence-specific off-target effects. The database and associated predictive tools enable the user to evaluate an siRNA's potential for inhibition and non-specific effects. The database is available at on the world wide web at siRNA.cgb.ki.se. Synthetic siRNA molecules, including shRNA molecules, can be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al., Nature 411:494-498 (2001); Elbashir, S. M., et al., Genes & Development 15:188-200 (2001); Harborth, J. et al., J. Cell Science 114:4557-4565 (2001); Masters, J. R. et al., Proc. Natl. Acad. Sci., USA 98:8012-8017 (2001); and Tuschl, T. et al., Genes & Development 13:3191-3197 (1999)).

Alternatively, several commercial RNA synthesis suppliers are available including, but not limited to, Proligo (Hamburg, Germany), Dharmacon Research (Lafayette, CO, USA), Pierce Chemical (part of Perbio Science, Rockford, IL, USA), Glen Research (Sterling, VA, USA), ChemGenes (Ashland, MA, USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al., Genes Dev. 16:948-958 (2002); McManus, M. T. et al., RNA 8:842-850 (2002); Paul, C. P. et al., Nat. Biotechnol. 20:505-508 (2002); Miyagishi, M. et al., Nat. Biotechnol. 20:497-500 (2002); Sui, G. et al., Proc. Natl. Acad. Sci., USA 99:5515-5520 (2002); Brummelkamp, T. et al., Cancer Cell 2:243 (2002); Lee, N. S., et al., Nat. Biotechnol. 20:500-505 (2002); Yu, J. Y., et al., Proc. Natl. Acad. Sci., USA 99:6047-6052 (2002); Zeng, Y., et al., Mol. Cell 9:1327-1333 (2002); Rubinson, D. A., et al., Nat. Genet. 33:401-406 (2003); Stewart, S. A., et al., RNA 9:493-501 (2003)).

In some embodiments of the various aspects described herein, the inhibitory nucleic acid is a guide nucleic acid (gNA). As used herein, the terms “guide nucleic acid,” “guide sequence,” “crRNA,” “guide RNA,” “single guide RNA,” “gRNA” or “CRISPR guide sequence” refer to a nucleic acid comprising a sequence that determines the specificity of an enzyme, e.g., the Cas DNA binding protein of a CRISPR/Cas system, to a polynucleotide target. The gNA can comprise a polynucleotide sequence with at least partial complementarity with a target nucleic acid sequence, sufficient to hybridize with the target nucleic acid sequence and to direct sequence-specific binding of an enzyme, e.g, a nuclease, to the target nucleic acid sequence.

In some embodiments, the enzyme directed by the gNA is a gene-editing protein, e.g., any nuclease that induces a nick or double-strand break into a desired recognition site. Such enzymes can be native or engineered. These breaks can then be repaired by the cell in one of two ways: non-homologous end joining and homology-directed repair (homologous recombination). In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence can be used as a template for repair of the cleaved target DNA sequence, resulting in the transfer of genetic information from the donor polynucleotide to the target DNA. Therefore, new nucleic acid material may be inserted/copied into the site. The modifications of the target DNA due to NHEJ and/or homology-directed repair can be used for gene correction, gene replacement, gene tagging, transgene insertion, nucleotide deletion, gene disruption, gene mutation, etc.

In one embodiment, the gene-editing protein is a CRISPR-associated nuclease. The native prokaryotic CRISPR-associated nuclease system comprises an array of short repeats with intervening variable sequences of constant length (i.e., clusters of regularly interspaced short palindromic repeats), and CRISPR-associated (“Cas”) nuclease proteins. The RNA of the transcribed CRISPR array is processed by a subset of the Cas proteins into small guide RNAs, which generally have two components as discussed below. There are at least three different systems: Type I, Type II and Type III. The enzymes involved in the processing of the RNA into mature crRNA are different in the 3 systems. In the native prokaryotic system, the guide RNA (“gRNA”) comprises two short, non-coding RNA species referred to as CRISPR RNA (“crRNA”) and trans-acting RNA (“tracrRNA”). In an exemplary system, the gRNA forms a complex with a nuclease, for example, a Cas nuclease. The gRNA: nuclease complex binds a target polynucleotide sequence having a protospacer adjacent motif (“PAM”) and a protospacer, which is a sequence complementary to a portion of the gRNA. The recognition and binding of the target polynucleotide by the gRNA: nuclease complex induces cleavage of the target.

Any CRISPR-associated nuclease can be used in the system and methods of the invention. CRISPR nuclease systems are known to those of skill in the art, e.g. Cas9, Cas12, Cas12a, or the like, see Patents/applications 8,993,233, US 2015/0291965, US 2016/0175462, US 2015/0020223, US 2014/0179770, 8,697,359; 8,771,945; 8,795,965; WO 2015/191693; U.S. Pat. No. 8,889,418; WO 2015/089351; WO 2015/089486; WO 2016/028682; WO 2016/049258; WO 2016/094867; WO 2016/094872; WO 2016/094874; WO 2016/112242; US 2016/0153004; US 2015/0056705; US 2016/0090607; US 2016/0029604; 8,865,406; 8,871,445; each of which are incorporated by reference in their entirety. The nuclease can also be a phage Cas nuclease, e.g., CasΦ (e.g., Pausch et al. Science 369:333-7 (2020); which is incorporated by reference herein in its entirety).

The full-length guide nucleic acid strand can be any length. For example, the guide nucleic acid strand can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments of the various aspects described herein, a nucleic acid strand is less than about 75, 50, 45, 40, 35, 30, 25, 20, 15, 12, or fewer nucleotides in length. For example, the guide nucleic acid sequence is 10-30 nucleotides long.

In addition to a sequence that is complementary to a target nucleic acid, in some embodiments, the gNA also comprises a scaffold sequence. Expression of a gNA encoding both a sequence complementary to a target nucleic acid and scaffold sequence has the dual function of both binding (hybridizing) to the target nucleic acid and recruiting the endonuclease to the target nucleic acid, which may result in site-specific CRISPR activity. In some embodiments, such a chimeric gNA may be referred to as a single guide RNA (sgRNA).

In some embodiments of the various aspects described herein, the guide nucleic acid is designed using a guide design tool (e.g., Benchling™; Broad Institute GPP™; CasOFFinder™; CHOPCHOP™; CRISPOR™; Deskgen™; E-CRISP™; Geneious™; GenHub™; GUIDES™ (e.g., for library design); Horizon Discovery™; IDT™; Off_Spotter™; and Synthego™; which are available on the world wide web).

In some embodiments of any of the aspects, the nucleic acid inhibitor comprises a nucleic modification. For example, the nucleic acid inhibitor is chemically modified to enhance stability or other beneficial characteristics. The nucleic acid inhibitors described herein may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages.

Modified nucleic acid backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified nucleic acid backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH—CH2-, —CH2-N(CH3)-O—CH2-[known as a methylene (methylimino) or MMI backbone], —CH2-O—N(CH3)-CH2-, —CH2-N(CH3)-N(CH3)-CH2- and —N(CH3)-CH2-CH2-[wherein the native phosphodiester backbone is represented as —O—P—O—CH2-].

In some embodiments of any one of the aspects, both the sugar and the internucleoside linkage, i.e., the backbone, of the nucleotide units in the nucleic acid inhibitor are replaced with novel groups. The base units are maintained for hybridization with an appropriate nucleic acid target compound. One such modification that has been shown to have excellent hybridization properties, is referred to as a peptide nucleic acid (PNA). In PNA compounds, the sugar backbone is replaced with an amide containing backbone, in particular an aminoethylglycine backbone. The nucleobases are retained and are bound directly or indirectly to aza nitrogen atoms of the amide portion of the backbone.

The nucleic acid inhibitors can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, OR. et al., (2007) Mol Canc Ther 6(3):833-843; Grunweller, A. et al., (2003) Nucleic Acids Research 31(12):3185-3193).

Modified nucleic acid inhibitors can also contain one or more substituted sugar moieties. For example, the nucleic acid inhibitor described herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁-C₁₀alkyl, C₂-C₁₀alkenyl or C₂-C₁₀alkynyl. Exemplary suitable modifications include O[(CH2)nO]mCH3, O(CH2)·nOCH3, O(CH2)nNH2, O(CH2) nCH3, O(CH2)nONH2, and O(CH2)nON[(CH2)nCH3)]2, where n and m are from 1 to about 10. In some embodiments of any of the aspects, dsRNAs include one of the following at the 2′ position: C₁-C₁₀alkyl, substituted C₁-C₁₀alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl. In some embodiments of any of the aspects, the modification includes a 2′ methoxyethoxy (2′-O—CH2CH2OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH2-O—CH2-N(CH2)2.

The inhibitory nucleic acid can also include nucleobase (often referred to in the art simply as “base”) modifications or substitutions. For example, the nucleic acid inhibitor can comprise a modified or non-natural nucleobase. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

In some embodiments of any one of the aspects described herein, the inhibitory nucleic acid comprises one or more ligands, moieties or conjugates that enhance the activity, cellular distribution, pharmacokinetic properties, or cellular uptake of the nucleic acid inhibitor. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et a1., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

The preparation modified nucleic acids are well known in the art and available to one of skill in the art.

### Antibody Inhibitors of NR4A Family

Antibodies that specifically bind NR4A family members can be used for inhibition in vivo, in vitro, or ex vivo. The NR4A family inhibitory activity of a given antibody, or, for that matter, any NR4A family inhibitor, can be assessed using methods known in the art or described herein. Specific binding is typically defined as binding that does not recognize other antigens, such as a protein, nucleotide, chemical residue, etc., at a detectable level in an assay used. Accordingly, in some embodiments of any one of the aspects described herein, the inhibitor of the NR4A family member is an antibody or an antigen binding fragment thereof. Generally, the antibody or the antigen binding fragment thereof binds an epitope on the NR4A family member such that the binding inhibits a function and/or activity of the NR4A family member.

Antibodies that can be used according to the methods described herein, include complete immunoglobulins, antigen binding fragments of immunoglobulins, as well as antigen binding proteins that comprise antigen binding domains of immunoglobulins. Antigen binding fragments of immunoglobulins include, for example, Fab, Fab′, F(ab′)2, scFv and dAbs. Modified antibody formats have been developed which retain binding specificity, but have other characteristics that may be desirable, including for example, bispecificity, multivalence (more than two binding sites), and compact size (e.g., binding domains alone). Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

Multiple single chain antibodies, each single chain having one VH and one VL domain covalently linked by a first peptide linker, can be covalently linked by at least one or more peptide linker to form multivalent single chain antibodies, which can be monospecific or multispecific. Each chain of a multivalent single chain antibody includes a variable light chain fragment and a variable heavy chain fragment, and is linked by a peptide linker to at least one other chain. The peptide linker is composed of at least fifteen amino acid residues. The maximum number of linker amino acid residues is approximately one hundred.

Two single chain antibodies can be combined to form a diabody, also known as a bivalent dimer. Diabodies have two chains and two binding sites, and can be monospecific or bispecific. Each chain of the diabody includes a VH domain connected to a VL domain. The domains are connected with linkers that are short enough to prevent pairing between domains on the same chain, thus driving the pairing between complementary domains on different chains to recreate the two antigen-binding sites.

Three single chain antibodies can be combined to form triabodies, also known as trivalent trimers. Triabodies are constructed with the amino acid terminus of a VL or VH domain directly fused to the carboxyl terminus of a VL or VH domain, i.e., without any linker sequence. The triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific.

Thus, antibodies useful in the methods described herein include, but are not limited to, naturally occurring antibodies, bivalent fragments such as (Fab′)2, monovalent fragments such as Fab, single chain antibodies, single chain Fv (scFv), single domain antibodies, multivalent single chain antibodies, diabodies, triabodies, and the like that bind specifically with an antigen.

Antibodies can also be raised against a nucleotide, polypeptide or portion of a polypeptide by methods known to those skilled in the art. Antibodies are readily raised in animals such as rabbits or mice by immunization with the gene product, or a fragment thereof. Immunized mice are particularly useful for providing sources of B cells for the manufacture of hybridomas, which in turn are cultured to produce large quantities of monoclonal antibodies. Antibody manufacture methods are described in detail, for example, in Harlow et al., 1988. While both polyclonal and monoclonal antibodies can be used in the methods described herein, it is preferred that a monoclonal antibody is used where conditions require increased specificity for a particular protein.

The term “intrabodies” as used herein, refers to a method wherein to target intracellular endogenous proteins as described in U.S. Pat. No. 6,004,940. Briefly, the method comprises the intracellular expression of an antibody capable of binding to the target. A DNA sequence is delivered to a cell, the DNA sequence contains a sufficient number of nucleotides coding for the portion of an antibody capable of binding to the target operably linked to a promoter that will permit expression of the antibody in the cell(s) of interest. The antibody is then expressed intracellularly and binds to the target, thereby disrupting the target from its normal actions.

“Antigen-binding fragments” include, inter alia, Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The terms Fab, Fc, pFc′, F(ab′) 2 and Fv are employed with standard immunological meanings [Klein, Immunology (John Wiley, New York, N.Y., 1982); Clark, W. R. (1986) The Experimental Foundations of Modern Immunology (Wiley & Sons, Inc., New York); Roitt, I. (1991) Essential Immunology, 7th Ed., (Blackwell Scientific Publications, Oxford)].

Antibody inhibitors of NR4A family members can include polyclonal and monoclonal antibodies and antigen-binding derivatives or fragments thereof.

In some embodiments of any one of the aspects described herein, the inhibitor is an anti-NR4A1 antibody or an antigen binding fragment thereof. Generally, the antibody or the antigen binding fragment thereof binds an epitope on the NR4A1 such that the binding inhibits a function and/or activity of the NR4A1. In some embodiments of any one of the aspects described herein, the antibody binds to polypeptide of comprising an amino acid sequence selected from the group consisting of SEQ ID NOs: 13-17. Exemplary anti-NR4A1 antibodies are commercially available and include, but are not limited to anti-NR41A, 25851-1-AP, ProteinTech; anti-NR41A, 12235-1-AP, ProteinTech; anti-NR41A, OriGene, Cat #: TA314017; anti-NR41A, Bioss, Cat #BS-3313R; anti-NR41A, Abcam, ab41917; anti-NR41A, Thermo-Fisher, Cat #53-5965-82; anti-NR41A, Invitrogen, #PA5-27274; anti-NR41A, Novus Biologicals, Cat #NB100-56745; anti-NR41A, Boster Bio,Cat #PA1676; anti-NR41A, Aviva Systems Biology, Cat #ARP31941_T100; anti-NR41A, Atlas Antibodies, Cat #HPA070142; anti-NR41A, Santa Cruz Biotechnologies, sc-365113; anti-NR41A, Creative Biolabs, NHP-AB411; anti-NR41A, EMD Millipore, Cat #ABE1454; anti-NR41A, ProSci, Cat. #27-450; and anti-NR41A, R&D Systems, Cat #PP-H1648-00.

In some embodiments of any one of the aspects described herein, the inhibitor is an anti-NR4A2 antibody or an antigen binding fragment thereof. Generally, the antibody or the antigen binding fragment thereof binds an epitope on the NR4A2 such that the binding inhibits a function and/or activity of the NR4A2. In some embodiments of any one of the aspects described herein, the antibody binds to polypeptide of comprising the amino acid sequence of SEQ ID NOs: 18-19. Exemplary anti-NR4A2 antibodies are commercially available and include, but are not limited to, anti-Nurr1, ab41917, Abcam; NR4A2 monoclonal antibody, 66878-1-Ig, ProteinTech; Nurr1 Polyclonal Antibody, CAT #: TA349421, OriGene; Nurr1 Monoclonal Antibody, Catalog #MA1-195, Invitrogen; Nurr1 Monoclonal Antibody, Catalog #H00004929-M08, Catalog #H00004929-M10, Catalog #H00004929-M07, Abnova; Anti-NR4A2 (Nurr1) antibody, Cat #682002, BioLegend; Nurr1/NGFI-B beta/NR4A2 Antibody, Cat #PP-N1404-00, Novus Biologicals; and anti-Nurr1 antibody, sc-376984, Santa Cruz Biotechnologies.

In some embodiments of any one of the aspects described herein, the inhibitor is an anti-NR4A3 antibody or an antigen binding fragment thereof. Generally, the antibody or the antigen binding fragment thereof binds an epitope on the NR4A3 such that the binding inhibits a function and/or activity of the NR4A3. In some embodiments of any one of the aspects described herein, the antibody binds to polypeptide of comprising the amino acid sequence of SEQ ID NOs: 20-22. Exemplary anti-NR4A3 antibodies are commercially available and include include, but are not limited to Recombinant Anti-NOR1/TEC antibody, ab259939, Abcam; NOR1 (NR4A3) Mouse Monoclonal Antibody, CAT #: TA804893, OriGene; NOR-1 Monoclonal Antibody, Catalog #MA5-26704, Invitrogen; NR4A3 Monoclonal Antibody, Catalog #H00008013-M02, Abnova; NR4A3/NOR1 Antibody, Cat #NBP2-46246, Novus Biologicals; Anti-NOR-1 Antibody, sc-393902, Santa Cruz Biotechnologies; and anti-NOR1 (NR4A3) Mouse Monoclonal Antibody, Cat #M02578, Boster Bio.

Methods of obtaining antibodies against an antigen are well known in the art and available to one of skill in the art.

### Small Molecule Inhibitors of NR4A Family

In some embodiments of any one of the aspects described herein, the inhibitor of the NR4A family member is a small molecule. Generally, the small molecule inhibitor binds with the NR4A family member and inhibits a function and/or activity of the NR4A family member. As used herein, the term “small molecules” refers to natural or synthetic molecules including, but not limited to, amino acids, peptides, peptidomimetics, polynucleotides, aptamers, nucleotide analogs, organic or inorganic compounds (i.e., including heterorganic and organometallic compounds), saccharides (e.g., mono, di, tri and polysaccharides), steroids, hormones, pharmaceutically derived drugs (e.g., synthetic or naturally occurring), lipids, derivatives of these (e.g., esters and salts of these), fragments of these, and conjugates of these. In some implementations the small molecules have a molecular weight less than about 5,000 Da, organic or inorganic compounds having a molecular weight less than about 2,500 Da, organic or inorganic compounds having a molecular weight less than about 1,000 Da, organic or inorganic compounds having a molecular weight less than about 500 Da. In some implementations the small molecule has a molecular weight of less than about 1000 Da.

In some embodiments of any one of the aspects described herein, the small molecule inhibitor is a 1,1-bis(3′-indolyl)-1-(p-substituted phenyl)methane (C-DIM) compounds are described in U.S. Pat. No. 7,232,843, content of which is incorporated herein by reference in its entirety. In some embodiments of any one of the aspects described herein, the inhibitor is selected from the group consisting of 1,1-bis(3′-indolyl)-1-(p-hydroxyphenyl)methane (DIM-C-pPhOH), 1,1-bis(3′-indolyl)-1-(3-chloro-4-hydroxy-5-methoxyphenyl)methane (DIM-C-pPhOh-3-Cl-5-OCH₃), 1,1-bis(3′-indolyl)-1-(3,5-dibromo-4-hydroxyphenyl)methane (DIM-C-pPhOH-3,5-Br2), camptothecin (CPT), a cyclooxygenase (COX)-2 inhibitor (celecoxib analogue SC-236), Erlotinib, Afatinib, Bosutinib, Dasatinib, and KU171309.

In some embodiments of any one of the aspects, the inhibitor is selected from the group consisting of DIM-C-pPhOH, DIM-C-pPhOh-3-Cl-5-OCH₃, DIM-C-pPhOH-3,5-Br2, camptothecin and celecoxib analogue SC-236. In some other embodiments of any one of the aspects described herein, the inhibitor is selected from the group consisting of Erlotinib, Afatinib, Bosutinib, Dasatinib, and KU171309.

In some embodiments of any one of the aspects described herein, the small molecule inhibitor inhabits NR4A1. For example, the inhibitor inhibits NR4A1 and is selected from the group consisting of DIM-C-pPhOH, DIM-C-pPhOh-3-Cl-5-OCH₃, DIM-C-pPhOH-3,5-Br2, camptothecin and celecoxib analogue SC-236.

In some embodiments of any one of the aspects described herein, the small molecule inhibitor inhibits NR4A2. For example, the inhibitor inhibits NR4A2 and is selected from the group consisting of Erlotinib, Afatinib, Bosutinib, Dasatinib, KU171309, C-DIM12 and DIM-C-pPhCl.

In some embodiments of any one of the aspects described herein, the smile molecule inhibitor inhibits NR4A3.

### Mutations

Hematopoietic stem cells (HSCs) and hematopoietic progenitor cells (HPCs) divide to produce blood cells by a continuous regeneration process. As the cells divide, they are prone to accumulating mutations, including deletions, insertions, and substitutions, that generally do not affect function. However, some mutations confer advantages in self-renewal, proliferation or both, resulting in clonal expansion of the cells comprising the mutations in question. The frequency of such somatic mutation events increases with age. The studies described herein demonstrate that preferential and progressive expansion of a subset of hematopoietic cells bearing somatic mutations in HSC genes, such as ASXL transcriptional regulator 1 (ASXL1), DNA (cytosine-5)-methyltransferase 3A (DNMT3A), isocitrate dehydrogenase (NADP(+)) 1(IDH1) and/or isocitrate dehydrogenase (NADP(+)) 2 (IDH 2) leads to increased production of inflammatory cytokines, such as IL-10 and/or TNF-P.

Accordingly, in some embodiments of any one of the aspects described herein, the subject has at least one mutation in a nucleic acid encoding ASXL1, a nucleic acid encoding DNMT3A, a nucleic acid encoding IDH1 or a nucleic acid encoding IDH 2. In some embodiments, the subject has at least one mutation in two of a nucleic acid encoding ASXL1, a nucleic acid encoding DNMT3A, a nucleic acid encoding IDH1 and a nucleic acid encoding IDH 2. In some embodiments, the subject has at least one mutation in three of a nucleic acid encoding ASXL1, a nucleic acid encoding DNMT3A, a nucleic acid encoding IDH1 and a nucleic acid encoding IDH 2. In some embodiments, the subject has at least one mutation in all four of a nucleic acid encoding ASXL1, a nucleic acid encoding DNMT3A, a nucleic acid encoding IDH1 and a nucleic acid encoding IDH 2. It is noted that the mutation in ASXL1, DMNT3A, IDH1 or IDH2 is in a subject's HSPC.

Exemplary types of mutations include, but are not limited to, substitutions, insertions and deletions. It is noted that the insertion and/or deletion mutations can be a frameshift mutation or an in-frame mutation. A frameshift mutation (also called a framing error or a reading frame shift) is a mutation caused by indels (insertion or deletions) of a number of nucleotides in a nucleic acid sequence that is not divisible by three. Due to the triplet nature of gene expression by codons, the insertion or deletion can change the reading frame (the grouping of the codons), resulting in a completely different translation from the original. By contrast, any insertion or deletion that is evenly divisible by three is termed an in-frame mutation.

Methods for determining mutations in a nucleic acid are well known in the art and include, but are not limited to, targeted mutational analysis, full sequence analysis, and deletion/duplication analysis. The mutation can be detected either directly, e.g., by genomic analysis or genetic probing, or indirectly, e.g., by measuring relative levels of gene products to detect abnormal expression levels. Enzyme expression levels can be determined in multiple manners, and quantitation is relative, based on a specific standard for each assay.

Methods for detecting the presence of a mutation in a gene of interest are known in the art. Suitable methods for determining whether or not a particular mutation in a gene exists include, e.g., Southern blot (see, e.g., Sambrook et al. {supra)), real-time PCR analysis (see, e.g., Oliver et a1. (2000) JMol Diagnostics 2(4}:202-208), nucleic acid array analysis, allele-specific PCR (e.g., quantitative allele-specific PCR), pyrosequencing, DNA sequencing (e.g., Sanger chemistry sequencing), or through the use of molecular beacons (e.g., Tyagi et al. (1998) Nat Biotechnol 16:49-53; Abravaya et al. (2003) Clin Chem Lab Med4\:468-474; and Mullah et al. (1999) Nucleosides & Nucleotides 18: 1311-1312, the disclosures of each of which are incorporated herein by reference in their entirety). Suitable conditions and methods for detecting nucleic acid mutations using nucleic acid arrays are described in, e.g., Lamy et al. (2006) Nucleic Acids Research 34(14): elOO; European Patent Application Publication No. 1234058; U.S. Patent Application Publication Nos. 20060008823 and 20030059813; and U.S. Pat. No. 6,410,231; the disclosures of each of which are incorporated herein by reference in their entirety.

Inventors have discovered inter alia that indel (insertion/deletion) mutations in the mutant HSPCs enhance the competitive advantage of the mutant clones to establish clonal dominance. Accordingly, in some embodiments of any one of the aspects described herein, the subject to be treated has an indel mutation in a nucleic acid encoding ASXL1, a nucleic acid encoding DNMT3A, a nucleic acid encoding IDH1 or a nucleic acid encoding IDH 2. For example, the subject to be treated has an in-frame indel mutation in at least one mutation in a nucleic acid encoding ASXL1, a nucleic acid encoding DNMT3A, a nucleic acid encoding IDH1 or a nucleic acid encoding IDH 2. In another non-limiting example, the subject to be treated has a frameshift indel mutation in at least one mutation in a nucleic acid encoding ASXL1, a nucleic acid encoding DNMT3A, a nucleic acid encoding IDH1 or a nucleic acid encoding IDH 2.

In some embodiments of any one of the aspects described herein, the subject to be treated comprises at least one mutation in a nucleic acid encoding ASXL1. For example, the subject has a frameshift mutation in a nucleic acid encoding ASXL1. In another non-limiting example, the subject has a frameshift mutation in a nucleic acid encoding ASXL1. The AXSL1, which is encoded on chromosome 20, is similar to the Drosophila additional sex combs gene, which encodes a chromatin-binding protein required for normal determination of segment identity in the developing embryo. The protein is a member of the Polycomb group of proteins, which are necessary for the maintenance of stable repression of homeotic and other loci. The protein is thought to disrupt chromatin in localized areas, enhancing transcription of certain genes while repressing the transcription of other genes. The protein encoded by this gene functions as a ligand-dependent co-activator for retinoic acid receptor in cooperation with nuclear receptor coactivator 1. Mutations in this gene are associated with myelodysplastic syndromes and chronic myelomonocytic leukemia. Alternative splicing results in multiple transcript variants. The ASXL1 referred to in this aspect, and all aspects and embodiments described herein in this application, comprises the nucleotide sequences of NCBI nucleotide sequence IDs: NC_000020.11 (SEQ ID NO: 23, AXSL1 genomic sequence), NM_015338.6 (SEQ ID NO: 24, AXSL1 isoform 1 mRNA), NM_001164603.1 (SEQ ID NO: 25, AXSL1 isoform 2 mRNA) and NM_001363734.1 (SEQ ID NO: 26, AXSL1 isoform 3 mRNA).

In some embodiments of any one of the aspects described herein, the subject to be treated comprises at least one mutation in a nucleic acid comprising a nucleotide sequence selected from the group consisting of SEQ ID NOs: 23-26. For example, the subject has a frameshift mutation in a nucleic acid comprising a nucleotide sequence selected from the group consisting of SEQ ID NOs: 23-26. In another example, the subject has an in-frame mutation in a nucleic acid comprising a nucleotide sequence selected from the group consisting of SEQ ID NOs: 23-26.

In some embodiments of any one of the aspects described herein, the subject to be treated comprises at least one mutation in a nucleic acid encoding DNMT3A. For example, the subject has a frameshift mutation in a nucleic acid encoding DNMT3A. In another non-limiting example, the subject has a frameshift mutation in a nucleic acid encoding DNMT3A. The DNMT3A, which is encoded on human chromosome 2. CpG methylation is an epigenetic modification that is important for embryonic development, imprinting, and X-chromosome inactivation. Studies in mice have demonstrated that DNA methylation is required for mammalian development. This gene encodes a DNA methyltransferase that is thought to function in de novo methylation, rather than maintenance methylation. The protein localizes to the cytoplasm and nucleus and its expression is developmentally regulated. The DMNT3A referred to in this aspect, and all aspects and embodiments described herein in this application, comprises the nucleotide sequences of NCBI nucleotide sequence IDs: NC_000002.12 (SEQ ID NO: 27, DNMT3A genomic sequence), NM_001320892.2 (SEQ ID NO: 28, DNMT3A isoform c mRNA), NM_001320893.1 (SEQ ID NO: 29, DNMT3A isoform d mRNA), NM_001375819.1 (SEQ ID NO: 30, DNMT3A isoform e mRNA), NM_022552.5 (SEQ ID NO: 31, DNMT3A isoform a mRNA), NM_153759.3 (SEQ ID NO: 32, DNMT3A isoform b mRNA), NM_175629.2 (SEQ ID NO: 33, DNMT3A isoform a mRNA), and NM_175630.1 (SEQ ID NO: 34, DNMT3A isoform c mRNA).

In some embodiments of any one of the aspects described herein, the subject to be treated comprises at least one mutation in a nucleic acid comprising a nucleotide sequence selected from the group consisting of SEQ ID NOs: 27-34. For example, the subject has a frameshift mutation in a nucleic acid comprising a nucleotide sequence selected from the group consisting of SEQ ID NOs: 27-34. In another example, the subject has an in-frame mutation in a nucleic acid comprising a nucleotide sequence selected from the group consisting of SEQ ID NOs: 27-34.

In some embodiments of any one of the aspects described herein, the subject to be treated comprises at least one mutation in a nucleic acid encoding IDH1. For example, the subject has a frameshift mutation in a nucleic acid encoding IDH1. In another non-limiting example, the subject has a frameshift mutation in a nucleic acid encoding IDH1. The IDH1, which is encoded on human chromosome 2. Isocitrate dehydrogenases catalyze the oxidative decarboxylation of isocitrate to 2-oxoglutarate. These enzymes belong to two distinct subclasses, one of which utilizes NAD(+) as the electron acceptor and the other NADP(+). Five isocitrate dehydrogenases have been reported: three NAD(+)-dependent isocitrate dehydrogenases, which localize to the mitochondrial matrix, and two NADP(+)-dependent isocitrate dehydrogenases, one of which is mitochondrial and the other predominantly cytosolic. Each NADP(+)-dependent isozyme is a homodimer. The protein encoded by this gene is the NADP(+)-dependent isocitrate dehydrogenase found in the cytoplasm and peroxisomes. It contains the PTS-1 peroxisomal targeting signal sequence. The presence of this enzyme in peroxisomes suggests roles in the regeneration of NADPH for intraperoxisomal reductions, such as the conversion of 2, 4-dienoyl-CoAs to 3-enoyl-CoAs, as well as in peroxisomal reactions that consume 2-oxoglutarate, namely the alpha-hydroxylation of phytanic acid. The cytoplasmic enzyme serves a significant role in cytoplasmic NADPH production. Alternatively, spliced transcript variants encoding the same protein have been found for this gene. The IDH1 referred to in this aspect, and all aspects and embodiments described herein in this application, comprises the nucleotide sequences of NCBI nucleotide sequence IDs: NC_000002.12 (SEQ ID NO: 35, IDH1 genomic sequence), NM_001282386.1 (SEQ ID NO: 36, IDH1 isoform 2 mRNA), NM_001282387.1 (SEQ ID NO: 37, IDH1 isoform 2 mRNA) and NM_005896.4 (SEQ ID NO: 38, IDH1 isoform 1 mRNA).

In some embodiments of any one of the aspects described herein, the subject to be treated comprises at least one mutation in a nucleic acid comprising a nucleotide sequence selected from the group consisting of SEQ ID NOs: 35-38. For example, the subject has a frameshift mutation in a nucleic acid comprising a nucleotide sequence selected from the group consisting of SEQ ID NOs: 35-38. In another example, the subject has an in-frame mutation in a nucleic acid comprising a nucleotide sequence selected from the group consisting of SEQ ID NOs: 35-38.

In some embodiments of any one of the aspects described herein, the subject to be treated comprises at least one mutation in a nucleic acid encoding IDH2. For example, the subject has a frameshift mutation in a nucleic acid encoding IDH2. In another non-limiting example, the subject has a frameshift mutation in a nucleic acid encoding IDH2. The IDH2, encoded on human chromosome 15. Isocitrate dehydrogenases catalyze the oxidative decarboxylation of isocitrate to 2-oxoglutarate. These enzymes belong to two distinct subclasses, one of which utilizes NAD(+) as the electron acceptor and the other NADP(+). Five isocitrate dehydrogenases have been reported: three NAD(+)-dependent isocitrate dehydrogenases, which localize to the mitochondrial matrix, and two NADP(+)-dependent isocitrate dehydrogenases, one of which is mitochondrial and the other predominantly cytosolic. Each NADP(+)-dependent isozyme is a homodimer. The protein encoded by this gene is the NADP(+)-dependent isocitrate dehydrogenase found in the mitochondria. It plays a role in intermediary metabolism and energy production. This protein may tightly associate or interact with the pyruvate dehydrogenase complex. Alternative splicing results in multiple transcript variants.

The IDH2 referred to in this aspect, and all aspects and embodiments described herein in this application, comprises the nucleotide sequences of NCBI nucleotide sequence IDs: NC_000015.10 (SEQ ID NO: 39, IDH2 genomic sequence), NM_002168.4 (SEQ ID NO: 40, IDH2 isoform 1 mRNA), NM_001289910.1 (SEQ ID NO: 41, IDH2 isoform 2 mRNA) and NM_001290114.2 (SEQ ID NO: 42, IDH2 isoform 3 mRNA).

In some embodiments of any one of the aspects described herein, the subject to be treated comprises at least one mutation in a nucleic acid comprising a nucleotide sequence selected from the group consisting of SEQ ID NOs: 39-42. For example, the subject has a frameshift mutation in a nucleic acid comprising a nucleotide sequence selected from the group consisting of SEQ ID NOs: 39-42. In another example, the subject has an in-frame mutation in a nucleic acid comprising a nucleotide sequence selected from the group consisting of SEQ ID NOs: 39-42.

A subject having a clonal hematopoiesis disease or disorder can suffer from conditions such as fatigue, shortness of breath, bruising or bleeding, infections due to low white blood cell count, pale skin, and anemia. Generally, the clonal hematopoiesis diseases and disorders are characterized by clonal expansion of mutant HSPCs, resulting in the depletion of wild-type HSPCs. Accordingly, some embodiments of any one of the aspects described herein, the subject to be treated is determined to have an increased level of mutant HSPCs and/or proliferation of mutant HSPCs cells relative to a reference level. For example, the method comprises a step of determining or obtaining results of an assay indicating a level of mutant HSPCs in a sample from the subject prior to onset of treatment.

In some embodiments of any one of the aspects described herein, the subject to be treated is determined to have an elevated level of at least one NR4A family member relative to a reference level. For example, the method comprises a step of determining or obtaining results of an assay indicating an expression level of at least member of a NR4A family member in a sample from the subject prior to onset of treatment.

It is noted that the reference level can be the level in a sample of similar sample type, sample processing, and/or obtained from a subject of similar age, sex and other demographic parameters as the sample/subject for which the level is to be determined. In some embodiments of any of the aspects, the reference level can be the level in a sample obtained from a reference subject of similar age as the subject for which the level is to be determined. In some embodiments of any of the aspects, the test sample and control reference sample are of the same type, that is, obtained from the same biological source, and comprising the same composition, e.g. the same number and type of cells.

In some embodiments of any of the aspects, the reference can be a level in a population of subjects who do not have or are not diagnosed as having, and/or do not exhibit signs or symptoms of a clonal hematopoiesis disease or disorder. In some embodiments of any of the aspects, the reference level can be a level in a population of subjects who do not contain mutations that confer competitive advantage to mutant HSPC, e.g., mutation in a nucleic acid encoding AXSL1, DMNT3A, IDH1 or IDH2. In some embodiments of any of the aspects, the reference level can also be a level in a control sample, a pooled sample of control individuals or a numeric value or range of values based on the same. In some embodiments of any of the aspects, the reference level can be the level in a sample obtained from the same subject at an earlier point in time, e.g., the methods described herein can be used to determine if a subject's sensitivity or response to a given therapy is changing over time.

A level which is more than a reference level can be a level which is greater by at least about 10%, at least about 20%, at least about 50%, at least about 60%, at least about 80%, at least about 90%, at least about 100%, at least about 200%, at least about 300%, at least about 500% or more than the reference level. In some embodiments of any of the aspects, a level which is more than a reference level can be a level which is statistically significantly greater than the reference level.

### Combination Therapies

The methods described herein can further comprise administering a second agent and/or treatment to the subject, e.g. as part of a combinatorial therapy. The additional therapy can be administered prior to, simultaneously with, or after administering the NR4A inhibitor.

The phrase “combination therapy” as used herein means administration a NR4A inhibitor and one or more additional therapies as part of a specific treatment regimen intended to provide a beneficial effect from the co-action of these. The beneficial effect of the combination includes, but is not limited to, pharmacokinetic or pharmacodynamic co-action resulting from the combination of therapeutic agents. Administration of these therapeutic agents in combination typically is carried out over a defined time period. The time period may be in minutes, hours, days or weeks depending upon the combination selected.

Combination therapy includes administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be done, for example, by administering to the subject a single pill having a fixed ratio of each therapeutic agent or in multiple, single pills for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered may or may not be important.

Combination therapy also can mean the administration of one or more inhibitors NR4A family in further combination with other compounds and non-drug therapies, such as, but not limited to, surgery or radiation treatment. Where the combination therapy further comprises radiation treatment, the radiation treatment may be conducted at any suitable time so long as a beneficial effect from the co-action of the combination of the therapeutic agents and radiation treatment is achieved.

If a subject is to be treated for pain or inflammation according to the methods described herein, the subject can also be administered a second agent and/or treatment known to be beneficial for subjects suffering from pain or inflammation. For example, the method can further comprise co-administering an anti-inflammatory agent to the subject. As used herein the term “anti-inflammatory agent” refers to a compound (including its analogs, derivatives, prodrugs and pharmaceutically salts) which can be used to treat inflammation or an inflammation related disease or disorder. Exemplary anti-inflammatory agents include, but are not limited to, the known steroidal anti-inflammatory and non-steroidal anti-inflammatory drugs (NSAIDs). Exemplary steroidal anti-inflammatory agents include but are not limited to 21-acetoxypregnenolone, alclometasone, algestone, amcinonide, beclomethasone, betamethasone, budesonide, chloroprednisone, clobetasol, clobetansone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furcate, paramethosone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, and analogues and derivatives thereof. Exemplary nonsteroidal anti-inflammatory agents include but are not limited to COX inhibitors (COX-1 or COX nonspecific inhibitors) and selective COX-2 inhibitors. Exemplary COX inhibitors include but are not limited to salicylic acid derivatives such as aspirin, sodium salicylate, choline magnesium trisalicylate, salicylate, diflunisal, sulfasalazine and olsalazine; para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin; anthranilic acids (fenamates) such as mefenamic acid and meloxicam; enolic acids such as the oxicams (piroxicam, meloxicam); alkanones such as nabumetone; and analogues and derivatives thereof. Exemplary COX-2 inhibitors include but are not limited to diaryl substituted furanones such as refecoxib; diaryl-substituted pyrazoles such as celecoxib; indole acetic acids such as etodolac and sulfonanilides such as nimesulide; and analogues and derivatives thereof.

In some embodiments, of the various aspects disclosed herein, the method can further comprise co-administering an immunomodulatory agent to the subject. Exemplary immunomodulatory agents include, but are not limited to, proteinaceous agents such as cytokines, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules and triple helices), small molecules, organic compounds, and inorganic compounds. In particular, immunomodulatory agents include, but are not limited to, methotrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide), T cell receptor modulators, cytokine receptor modulators, and modulators mast cell modulators.

Examples of T cell receptor modulators include, but are not limited to, anti-T cell receptor antibodies (e.g., anti-CD4 antibodies (e.g., cM-T412 (Boeringer), IDEC-CE9.1.R™ (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies (e.g., siplizumab (MedImmune, Inc., International Publication Nos. WO 02/098370 and WO 02/069904)), anti-CD11a antibodies (e.g., Xanelim (Genentech)), and anti-B7 antibodies (e.g., IDEC-114) (IDEC))), CTLA4-immunoglobulin, and LFA-3TIP (Biogen, International Publication No. WO 93/08656 and U.S. Pat. No. 6,162,432).

Examples of cytokine receptor modulators include, but are not limited to, soluble cytokine receptors (e.g., the extracellular domain of a TNF-alpha receptor or a fragment thereof, the extracellular domain of an IL-1.beta. receptor or a fragment thereof, and the extracellular domain of an IL-6 receptor or a fragment thereof), cytokines or fragments thereof (e.g., interleukin IL-2, IL-3, IL-4, IL-5, IL-6, IL-7, IL-8, IL-9, IL-10, IL-11, IL-12, IL-13, IL-15, IL-23, TNF-alpha, TNF-beta, interferon (IFN)-alpha, IFN-beta, IFN-gamma, and GM-CSF), anti-cytokine receptor antibodies (e.g., anti-IFN receptor antibodies, anti-IL-2 receptor antibodies (e.g., Zenapax™ (Protein Design Labs)), anti-IL-3 receptor antibodies, anti-IL-4 receptor antibodies, anti-IL-6 receptor antibodies, anti-IL-10 receptor antibodies, anti-IL-12 receptor antibodies, anti-IL-13 receptor antibodies, anti-IL-15 receptor antibodies, and anti-IL-23 receptor antibodies), anti-cytokine antibodies (e.g., anti-IFN antibodies, anti-TNF-.alpha. antibodies, anti-IL-1.beta. antibodies, anti-IL-3 antibodies, anti-IL-6 antibodies, anti-IL-8 antibodies (e.g., ABX-IL-8 (Abgenix)), anti-IL-12 antibodies, anti-IL-13 antibodies, anti-IL-15 antibodies, and anti-IL-23 antibodies).

In some embodiments, a cytokine receptor modulator is IL-3, IL-4, IL-10, or a fragment thereof. In another embodiment, a cytokine receptor modulator is an anti-IL-1-beta antibody, anti-IL-6 antibody, anti-IL-12 receptor antibody, or anti-TNF-alpha antibody. In one embodiment, a TNF-alpha antagonist used in the compositions and methods of the invention is a soluble TNF-alpha receptor. In some embodiments, a TNF-alpha antagonist used in the compositions and methods of the invention is etanercept (ENBREL™; Immunex) or a fragment, derivative or analog thereof. In another embodiment, a TNF-alpha antagonist used in the compositions and methods of the invention is an antibody that immunospecifically binds to TNF-.alpha.. In some embodiments, a TNF-alpha antagonist used in the compositions and methods of the invention is infliximab (REMICADE™; Centacor) a derivative, analog or antigen-binding fragment thereof. In another embodiment, a cytokine receptor modulator is the extracellular domain of a TNF-alpha receptor or a fragment thereof. In certain embodiments, a cytokine receptor modulator is not a TNF-alpha antagonist.

In one embodiment, a cytokine receptor modulator is a mast cell modulator. In an alternative embodiment, a cytokine receptor modulator is not a mast cell modulator. Examples of mast cell modulators include, but are not limited to stem cell factor (c-kit receptor ligand) inhibitors (e.g., mAb 7H6, mAb 8H7a, pAb 1337, FK506, CsA, dexamthasone, and fluconcinonide), c-kit receptor inhibitors (e.g., STI 571 (formerly known as CGP 57148B)), mast cell protease inhibitors (e.g., GW-45, GW-58, wortmannin, LY 294002, calphostin C, cytochalasin D, genistein, KT5926, staurosproine, and lactoferrin), relaxin (“RLX”), IgE antagonists (e.g., antibodies rhuMAb-E25 omalizumab, HMK-12 and 6HD5, and mAB Hu-901), IL-3 antagonists, IL-4 antagonists, IL-10 antagonists, and TGF-beta.

In some embodiments, of the various aspects disclosed herein, the method can further comprise co-administering an additional anti-cancer therapy to the subject. For example, administering a standard of care chemotherapeutic to the subject. Non-limiting examples of a standard of care chemotherapeutics or other anti-cancer therapy can include radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g. calicheamicin, especially calicheamicin gammalI and calicheamicin omegaIl (see, e.g. Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g. erlotinib (Tarceva®)) and VEGF-A that reduce cell proliferation and pharmaceutically acceptable salts, acids or derivatives of any of the above. Additional anti-cancer treatment can further include the use of radiation or radiation therapy. Further, the additional anti-cancer treatment can also include the use of surgical treatments.

### Inhibiting Clonal Expansion

In another aspect provided herein is a method for inhibiting clonal expansion of hematopoietic stem and progenitor cells. The method comprises administering an effective amount of an inhibitor of a member of the NR4A family to a HSPC. In some embodiments of any one of the aspects described herein, the method comprises administering an effective amount of an inhibitor of a member of the NR4A family to a HSPC, wherein the NR4A family member is selected from the group consisting of NR4A1, NR4A2 and NR4A3. In some preferred embodiments, the NR4A family member is NR4A1.

It is noted that administering to the HSPC can be in vitro or in-vivo. Methods for administering a compound to a cell are well known and available to one of skill in the art. As used herein, administering the compound to the cell means contacting the cell with the compound so that the compound is taken up by the cell. Generally, the cell can be contacted with the compound in a cell culture e.g., in vitro or ex vivo, or the compound can be administrated to a subject, e.g., in vivo. The term “contacting” or “contact” as used herein in connection with contacting a cell includes subjecting the cells to an appropriate culture media, which comprises NR4A inhibitor. Where the cell is in vivo, “contacting” or “contact” includes administering the Inhibitor, e.g., in a pharmaceutical composition to a subject via an appropriate administration route such that the compound contacts the cell in vivo.

For example, when the cell is in vitro, said administering to the cell can include subjecting the cell to an appropriate culture media which comprises the indicated compound. Where the cell is in vivo, said administering to the cell includes administering the compound to a subject via an appropriate administration route such that the compound is administered to the cell in vivo.

In some embodiments of any one of the aspects, the HSPC to which the NR4A inhibitor is administered has an elevated level of at least one NR4A family member relative to a reference level. For example, the HSPC has an elevated level of NR4A1 relative to a reference level. Thus, in some embodiments, the method further comprises measuring or determining a level of at least one NR4A family member relative to a reference level prior to administering the NR4A inhibitor to the cell.

In some embodiments of any one of the aspects, the HSPC to which the NR4A inhibitor is administered has at least one mutation in a nucleic acid encoding ASXL1, a nucleic acid encoding DNMT3A, a nucleic acid encoding IDH1 or a nucleic acid encoding IDH 2. In some embodiments, the HSPC has at least one mutation in two of a nucleic acid encoding ASXL1, a nucleic acid encoding DNMT3A, a nucleic acid encoding IDH1 and a nucleic acid encoding IDH 2. In some embodiments, the HSPC has at least one mutation in three of a nucleic acid encoding ASXL1, a nucleic acid encoding DNMT3A, a nucleic acid encoding IDH1 and a nucleic acid encoding IDH 2. In some embodiments, the HSPC has at least one mutation in all four of a nucleic acid encoding ASXL1, a nucleic acid encoding DNMT3A, a nucleic acid encoding IDH1 and a nucleic acid encoding IDH 2. In some embodiments, the method further comprises detecting or assaying, prior to administering the NR4A inhibitor to the cell, for a mutation in a nucleic acid encoding ASXL1, a nucleic acid encoding DNMT3A, a nucleic acid encoding IDH1 or a nucleic acid encoding IDH 2.

In some embodiments of any one of the aspects described herein, the HSPC has at least one mutation in a nucleic acid encoding ASXL 1. For example, the HSPC has a frameshift mutation in a nucleic acid encoding ASXL1. In another non-limiting example, the HSPC has a frameshift mutation in a nucleic acid encoding ASXL1. In some embodiments of any one of the aspects described herein, the HSPC has at least one mutation in a nucleic acid comprising a nucleotide sequence selected from the group consisting of SEQ ID NOs: 23-26. For example, the HSPC has a frameshift mutation in a nucleic acid comprising a nucleotide sequence selected from the group consisting of SEQ ID NOs: 23-26. In another example, the HSPC has an in-frame mutation in a nucleic acid comprising a nucleotide sequence selected from the group consisting of SEQ ID NOs: 23-26.

In some embodiments of any one of the aspects described herein, the HSPC to which the NR4A inhibitor is administered has at least one mutation in a nucleic acid encoding DNMT3A. For example, the HSPC has a frameshift mutation in a nucleic acid encoding DNMT3A. In another non-limiting example, the HSPC has a frameshift mutation in a nucleic acid encoding DNMT3A. In some embodiments of any one of the aspects described herein, the HSPC to which the NR4A inhibitor is administered comprises at least one mutation in a nucleic acid comprising a nucleotide sequence selected from the group consisting of SEQ ID NOs: 27-34. For example, the HSPC has a frameshift mutation in a nucleic acid comprising a nucleotide sequence selected from the group consisting of SEQ ID NOs: 27-34. In another example, the HSPC has an in-frame mutation in a nucleic acid comprising a nucleotide sequence selected from the group consisting of SEQ ID NOs: 27-34.

In some embodiments of any one of the aspects described herein, the HSPC to which the NR4A inhibitor is administered comprises at least one mutation in a nucleic acid encoding IDH1. For example, the HSPC has a frameshift mutation in a nucleic acid encoding IDH1. In another non-limiting example, the HSPC has a frameshift mutation in a nucleic acid encoding IDH1. In some embodiments of any one of the aspects described herein, the HSPC to which the NR4A inhibitor is administered comprises at least one mutation in a nucleic acid comprising a nucleotide sequence selected from the group consisting of SEQ ID NOs: 35-38. For example, the HSPC has a frameshift mutation in a nucleic acid comprising a nucleotide sequence selected from the group consisting of SEQ ID NOs: 35-38. In another example, the HSPC has an in-frame mutation in a nucleic acid comprising a nucleotide sequence selected from the group consisting of SEQ ID NOs: 35-38.

In some embodiments of any one of the aspects described herein, the HSPC to which the NR4A inhibitor is administered comprises at least one mutation in a nucleic acid encoding IDH2. For example, the HSPC has a frameshift mutation in a nucleic acid encoding IDH2. In another non-limiting example, the HSPC has a frameshift mutation in a nucleic acid encoding IDH2. In some embodiments of any one of the aspects described herein, the HSPC to which the NR4A inhibitor is administered comprises at least one mutation in a nucleic acid comprising a nucleotide sequence selected from the group consisting of SEQ ID NOs: 39-42. For example, the HSPC has a frameshift mutation in a nucleic acid comprising a nucleotide sequence selected from the group consisting of SEQ ID NOs: 39-42. In another example, the HSPC has an in-frame mutation in a nucleic acid comprising a nucleotide sequence selected from the group consisting of SEQ ID NOs: 39-42.

### Methods for Determining Expression Levels

In some embodiments of any of the aspects, the methods described herein are directed to determination of the expression level of a clonal population of HSPCs in a biological sample of a subject.

In some embodiments of any of the aspects, the methods described herein are directed to determination of the expression level of a gene product (e.g. protein and/or gene transcript such as mRNA) in a biological sample from a subject.

In some embodiments of any of the aspects, measurement of the level of a target and/or detection of the level or presence of a target, e.g. of an expression product (nucleic acid or polypeptide of one of the genes described herein) or a mutation can comprise a transformation. As used herein, the term “transforming” or “transformation” refers to changing an object or a substance, e.g., biological sample, nucleic acid or protein, into another substance. The transformation can be physical, biological or chemical. Exemplary physical transformation includes, but is not limited to, pre-treatment of a biological sample, e.g., from whole blood to blood serum by differential centrifugation. A biological/chemical transformation can involve the action of at least one enzyme and/or a chemical reagent in a reaction. For example, a DNA sample can be digested into fragments by one or more restriction enzymes, or an exogenous molecule can be attached to a fragmented DNA sample with a ligase. In some embodiments of any of the aspects, a DNA sample can undergo enzymatic replication, e.g., by polymerase chain reaction (PCR).

Transformation, measurement, and/or detection of a target molecule, e.g. a mRNA or polypeptide can comprise contacting a sample obtained from a subject with a reagent (e.g. a detection reagent) which is specific for the target, e.g., a target-specific reagent. In some embodiments of any of the aspects, the target-specific reagent is detectably labeled. In some embodiments of any of the aspects, the target-specific reagent is capable of generating a detectable signal. In some embodiments of any of the aspects, the target-specific reagent generates a detectable signal when the target molecule is present.

Methods to measure gene expression products are known to a skilled artisan. Such methods to measure gene expression products, e.g., protein level, include ELISA (enzyme linked immunosorbent assay), western blot, immunoprecipitation, and immunofluorescence using detection reagents such as an antibody or protein binding agents. Alternatively, a peptide can be detected in a subject by introducing into a subject a labeled anti-peptide antibody and other types of detection agent. For example, the antibody can be labeled with a detectable marker whose presence and location in the subject is detected by standard imaging techniques.

In some embodiments of any of the aspects, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience a change of color, upon encountering the targeted molecules. In some instances, signal amplification can be integrated into the particular protocol, wherein a secondary antibody, that includes the marker stain or marker signal, follows the application of a primary specific antibody.

In some embodiments of any of the aspects, the assay can be a Western blot analysis. Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. These methods also require a considerable amount of cellular material. The analysis of 2D SDS-PAGE gels can be performed by determining the intensity of protein spots on the gel, or can be performed using immune detection. In other embodiments, protein samples are analyzed by mass spectroscopy.

Immunological tests can be used with the methods and assays described herein and include, for example, competitive and non-competitive assay systems using techniques such as Western blots, radioimmunoassay (RIA), ELISA (enzyme linked immunosorbent assay), “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiments of any of the aspects, the immunoassay can be a quantitative or a semi-quantitative immunoassay.

An immunoassay is a biochemical test that measures the concentration of a substance in a biological sample, typically a fluid sample such as blood or serum, using the interaction of an antibody or antibodies to its antigen. The assay takes advantage of the highly specific binding of an antibody with its antigen. For the methods and assays described herein, specific binding of the target polypeptides with respective proteins or protein fragments, or an isolated peptide, or a fusion protein described herein occurs in the immunoassay to form a target protein/peptide complex. The complex is then detected by a variety of methods known in the art. An immunoassay also often involves the use of a detection antibody.

Enzyme-linked immunosorbent assay, also called ELISA, enzyme immunoassay or EIA, is a biochemical technique used mainly in immunology to detect the presence of an antibody or an antigen in a sample. The ELISA has been used as a diagnostic tool in medicine and plant pathology, as well as a quality control check in various industries.

In one embodiment, an ELISA involving at least one antibody with specificity for the particular desired antigen (e.g., any of the targets as described herein) can also be performed. A known amount of sample and/or antigen is immobilized on a solid support (usually a polystyrene micro titer plate). Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against a target polypeptide or fragment thereof are coated on the solid phase of multi-well plate, i.e., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., a blood sample) from a subject is mixed with a known amount of desired antigen (e.g., a known volume or concentration of a sample comprising a target polypeptide) together with the horseradish peroxidase labeled antibodies and the mixture is then added to coated wells to form competitive combination. After incubation, if the polypeptide level is high in the sample, a complex of labeled antibody reagent-antigen will form. This complex is free in solution and can be washed away. Washing the wells will remove the complex. Then the wells are incubated with TMB (3, 3′, 5, 5′-tetramethylbenzidene) color development substrate for localization of horseradish peroxidase-conjugated antibodies in the wells. There will be no color change or little color change if the target polypeptide level is high in the sample. If there is little or no target polypeptide present in the sample, a different complex in formed, the complex of solid support bound antibody reagents-target polypeptide. This complex is immobilized on the plate and is not washed away in the wash step. Subsequent incubation with TMB will produce significant color change. Such a competitive ELSA test is specific, sensitive, reproducible and easy to operate.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

In one embodiment, the levels of a polypeptide in a sample can be detected by a lateral flow immunoassay test (LFIA), also known as the immunochromatographic assay, or strip test. LFIAs are a simple device intended to detect the presence (or absence) of antigen, e.g. a polypeptide, in a fluid sample. There are currently many LFIA tests used for medical diagnostics, either for home testing, point of care testing, or laboratory use. LFIA tests are a form of immunoassay in which the test sample flows along a solid substrate via capillary action. After the sample is applied to the test strip it encounters a colored reagent (generally comprising antibody specific for the test target antigen) bound to microparticles which mixes with the sample and transits the substrate encountering lines or zones which have been pretreated with another antibody or antigen. Depending upon the level of target polypeptides present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water, and/or homogenized tissue samples etc. Strip tests are also known as dip stick tests, the name bearing from the literal action of “dipping” the test strip into a fluid sample to be tested. LFIA strip tests are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles which are labeled with antibodies raised to the target antigen. The test line will also contain antibodies to the same target, although it may bind to a different epitope on the antigen. The test line will show as a colored band in positive samples. In some embodiments of any of the aspects, the lateral flow immunoassay can be a double antibody sandwich assay, a competitive assay, a quantitative assay or variations thereof. Competitive LFIAs are similar to competitive ELISA. The sample first encounters colored particles which are labeled with the target antigen or an analogue. The test line contains antibodies to the target/its analogue. Unlabelled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871; 6,565,808, U.S. patent application Ser. No. 10/278,676; U.S. Ser. No. 09/579,673 and U.S. Ser. No. 10/717,082, which are incorporated herein by reference in their entirety, are non-limiting examples of such lateral flow test devices. Examples of patents that describe the use of “dip stick” technology to detect soluble antigens via immunochemical assays include, but are not limited to U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teachings of this “dip stick” technology for the detection of polypeptides using antibody reagents as described herein.

Other techniques can be used to detect the level of a polypeptide in a sample. One such technique is the dot blot, an adaptation of Western blotting (Towbin et at., Proc. Nat. Acad. Sci. 76:4350 (1979)). In a Western blot, the polypeptide or fragment thereof can be dissociated with detergents and heat, and separated on an SDS-PAGE gel before being transferred to a solid support, such as a nitrocellulose or PVDF membrane. The membrane is incubated with an antibody reagent specific for the target polypeptide or a fragment thereof. The membrane is then washed to remove unbound proteins and proteins with non-specific binding. Detectably labeled enzyme-linked secondary or detection antibodies can then be used to detect and assess the amount of polypeptide in the sample tested. A dot blot immobilizes a protein sample on a defined region of a support, which is thenprobed with antibody and labelled secondary antibody as in Western blotting. The intensity of the signal from the detectable label in either format corresponds to the amount of enzyme present, and therefore the amount of polypeptide. Levels can be quantified, for example by densitometry.

In some embodiments of any of the aspects, the level of a target can be measured, by way of non-limiting example, by Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; immunofluoresence assay; mass spectroscopy and/or immunoelectrophoresis assay.

In certain embodiments, the expression levels as described herein can be determined by determining the level of messenger RNA (mRNA) expression of the genes described herein. Such molecules can be isolated, derived, or amplified from a biological sample, such as a blood sample. Techniques for the detection of mRNA expression is known by persons skilled in the art, and can include but not limited to, PCR procedures, RT-PCR, quantitative RT-PCR Northern blot analysis, differential gene expression, RNAse protection assay, microarray based analysis, next-generation sequencing; hybridization methods, etc.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments of any of the aspects, the level of an mRNA can be measured by a quantitative sequencing technology, e.g. a quantitative next-generation sequence technology. Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing, dideoxy chain termination, high-throughput sequencing, next generation sequencing, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In some embodiments of any of the aspects, one or more of the reagents (e.g. an antibody reagent and/or nucleic acid probe) described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies and nucleic acid probes) are well known in the art.

In some embodiments of any of the aspects, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

The term “sample” or “test sample” as used herein denotes a sample taken or isolated from a biological organism, e.g., a blood or plasma sample from a subject. In some embodiments of any of the aspects, the present invention encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; sperm; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject. In some embodiments of any of the aspects, the test sample can be peripheral blood sample and the kidney marrow.

The test sample can be obtained by removing a sample from a subject, but can also be accomplished by using a previously isolated sample (e.g. isolated at a prior timepoint and isolated by the same or another person).

In some embodiments of any of the aspects, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments of any of the aspects, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments of any of the aspects, the test sample is a clarified test sample, for example, by centrifugation and collection of a supernatant comprising the clarified test sample. In some embodiments of any of the aspects, a test sample can be a pre-processed test sample, for example, supernatant or filtrate resulting from a treatment selected from the group consisting of centrifugation, filtration, thawing, purification, and any combinations thereof. In some embodiments of any of the aspects, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments of any of the aspects, the method described herein can further comprise a step of obtaining or having obtained a test sample from a subject. In some embodiments of any of the aspects, the subject can be a human subject. In some embodiments of any of the aspects, the subject can be a subject in need of treatment for (e.g. having or diagnosed as having clonal hematopoiesis, myelodysplasia, and leukemia) or a subject at risk of or at increased risk of developing clonal hematopoiesis, myelodysplasia, and leukemia as described elsewhere herein.

In some embodiments, the methods described herein relate to treating a subject having or diagnosed as having clonal hematopoiesis, myelodysplasia, and leukemia. In some embodiments of any of the aspects, the subject is a current or former blood disease patient. In some embodiments of any of the aspects, the subject has been exposed to chemotherapy, radiation, viral infection, or certain chemicals. In some embodiments of any of the aspects, the subject has or is diagnosed as having clonal hematopoiesis, myelodysplasia, and leukemia.

### Administration

In some embodiments, the methods described herein relate to treating a subject having or diagnosed as having a clonal hematopoietic disease or disorder, such as clonal hematopoiesis, myelodysplasia, or leukemia. Subjects having a clonal hematopoietic disease or disorder, such as clonal hematopoiesis, myelodysplasia, or leukemia can be identified by a physician using current methods of diagnosing such diseases and disorders. Symptoms and/or complications of clonal hematopoiesis, myelodysplasia, and leukemia which characterize these conditions and aid in diagnosis are well known in the art and include but are not limited to, anemia, bruising and bleeding, infections due to low levels of white blood cells, fevers, drenching night sweats, unintentional weight loss, and fatigue. Tests that may aid in a diagnosis of, e.g. clonal hematopoiesis, myelodysplasia, and leukemia include, but are not limited to, blood tests, bone marrow aspiration and biopsy, and lumbar puncture. A family history of leukemia, mutation in the GATA2 gene, TERC gene, or TERT gene for myelodysplasia, or exposure to risk factors for clonal hematopoiesis, myelodysplasia, and leukemia (e.g. exposure to chemotherapy, radiation, viral infection, or certain chemicals (e.g., benzene)) can also aid in determining if a subject is likely to have clonal hematopoiesis, myelodysplasia, and leukemia or in making a diagnosis of clonal hematopoiesis, myelodysplasia, and leukemia.

### Routes of Administration

It is noted that the terms “administered” and “subjected” are used interchangeably in the context of treatment of a disease or disorder. In jurisdictions that forbid the patenting of methods that are practiced on the human body, the meaning of “administering” of a composition to a human subject shall be restricted to prescribing a controlled substance that a human subject will be administer to the subject by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, administration will generally be local rather than systemic.

### Pharmaceutical Compositions

For administration to a subject, the NR4A inhibitor can be provided in pharmaceutically acceptable compositions. These pharmaceutically acceptable compositions comprise a NR4A inhibitor, formulated together with one or more pharmaceutically acceptable carriers (additives) and/or diluents. As described in detail below, the pharmaceutical compositions described herein can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Examples of solid carriers include starch, sugar, bentonite, silica, and other commonly used carriers. Further non-limiting examples of carriers and diluents which can be used in the formulations comprising a NR4A inhibitor include saline, syrup, dextrose, and water.

Pharmaceutically-acceptable antioxidants include, but are not limited to, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lectithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

The NR4A inhibitor can be formulated in a gelatin capsule, in tablet form, dragee, syrup, suspension, topical cream, suppository, injectable solution, or kits for the preparation of syrups, suspension, topical cream, suppository or injectable solution just prior to use. Also, compounds can be included in composites, which facilitate its slow release into the blood stream, e.g., silicon disc, polymer beads.

The formulations can conveniently be presented in unit dosage form and may be prepared by any of the methods well known in the art of pharmacy. Techniques, excipients and formulations generally are found in, e.g., Remington's Pharmaceutical Sciences, Mack Publishing Co., Easton, Pa. 1985, 17th edition, Nema et al., PDA J. Pharm. Sci. Tech. 1997 51:166-171. Methods to make invention formulations include the step of bringing into association or contacting an ActRIIB compound with one or more excipients or carriers. In general, the formulations are prepared by uniformly and intimately bringing into association one or more compounds with liquid excipients or finely divided solid excipients or both, and then, if appropriate, shaping the product.

The preparative procedure may include the sterilization of the pharmaceutical preparations. The compounds may be mixed with auxiliary agents such as lubricants, preservatives, stabilizers, salts for influencing osmotic pressure, etc., which do not react deleteriously with the compounds.

Examples of injectable forms include solutions, suspensions and emulsions. Injectable forms also include sterile powders for extemporaneous preparation of injectable solutions, suspensions or emulsions. The compounds of the present invention can be injected in association with a pharmaceutical carrier such as normal saline, physiological saline, bacteriostatic water, Cremophor™ EL (BASF, Parsippany, N.J.), phosphate buffered saline (PBS), Ringer's solution, dextrose solution, ethanol, polyol (e.g., glycerol, propylene glycol, and liquid polyethylene glycol), vegetable oils, and suitable mixtures thereof, and other aqueous carriers known in the art. Appropriate non-aqueous carriers may also be used and examples include fixed oils and ethyl oleate. In all cases, the composition must be sterile and should be fluid to the extent that easy syringability exists. It must be stable under the conditions of manufacture and storage and must be preserved against the contaminating action of microorganisms such as bacteria and fungi. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin. A suitable carrier is 5% dextrose in saline. Frequently, it is desirable to include additives in the carrier such as buffers and preservatives or other substances to enhance isotonicity and chemical stability.

In some embodiments, the NR4A inhibitor can be administrated encapsulated within liposomes. The manufacture of such liposomes and insertion of molecules into such liposomes being well known in the art, for example, as described in U.S. Pat. No. 4,522,811. Liposomal suspensions (including liposomes targeted to particular cells, e.g., a pituitary cell) can also be used as pharmaceutically acceptable carriers.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185; content of each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments, the NR4A inhibitor are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

In the case of oral ingestion, excipients useful for solid preparations for oral administration are those generally used in the art, and the useful examples are excipients such as lactose, sucrose, sodium chloride, starches, calcium carbonate, kaolin, crystalline cellulose, methyl cellulose, glycerin, sodium alginate, gum arabic and the like, binders such as polyvinyl alcohol, polyvinyl ether, polyvinyl pyrrolidone, ethyl cellulose, gum arabic, shellac, sucrose, water, ethanol, propanol, carboxymethyl cellulose, potassium phosphate and the like, lubricants such as magnesium stearate, talc and the like, and further include additives such as usual known coloring agents, disintegrators such as alginic acid and Primogel™, and the like. The compounds can be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these compounds may be incorporated with excipients and used in the form of tablets, capsules, elixirs, suspensions, syrups, and the like. Such compositions and preparations should contain at least 0.1% of compound. The percentage of the agent in these compositions may, of course, be varied and may conveniently be between about 2% to about 60% of the weight of the unit. The amount of compound in such therapeutically useful compositions is such that a suitable dosage will be obtained. Preferred compositions according to the present invention are prepared so that an oral dosage unit contains between about 100 and 2000 mg of compound. Examples of bases useful for formulation of suppositories are oleaginous bases such as cacao butter, polyethylene glycol, lanolin, fatty acid triglycerides, witepsol (trademark, Dynamite Nobel Co. Ltd.) and the like. Liquid preparations may be in the form of aqueous or oleaginous suspension, solution, syrup, elixir and the like, which can be prepared by a conventional way using additives. The compositions can be given as a bolus dose, to maximize the circulating levels for the greatest length of time after the dose. Continuous infusion may also be used after the bolus dose.

The NR4A inhibitor can also be administrated directly to the airways in the form of an aerosol. For administration by inhalation, the compounds in solution or suspension can be delivered in the form of an aerosol spray from pressured container or dispenser which contains a suitable propellant, e.g., a gas such as carbon dioxide, or hydrocarbon propellant like propane, butane or isobutene. The compounds can also be administrated in a no-pressurized form such as in an atomizer or nebulizer.

In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

Representative intranasal formulations are described in, for example, U.S. Pat. Nos. 4,476,116, 5,116,817 and 6,391,452. Formulations that include a compound of Formulae (I), (II), (III) or (IV) are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions

The NR4A inhibitor can also be administered parenterally. Solutions or suspensions of these compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

It may be advantageous to formulate oral or parenteral compositions in dosage unit form for ease of administration and uniformity of dosage. As used herein, “dosage unit” refers to physically discrete units suited as unitary dosages for the subject to be treated; each unit containing a predetermined quantity of compound calculated to produce the desired therapeutic effect in association with the required pharmaceutical carrier.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds are formulated into ointments, salves, gels, or creams as generally known in the art.

For oral or enteral formulations as disclosed herein for use with the present invention, tablets can be formulated in accordance with conventional procedures employing solid carriers well-known in the art. Capsules employed for oral formulations to be used with the methods of the present invention can be made from any pharmaceutically acceptable material, such as gelatin or cellulose derivatives. Sustained release oral delivery systems and/or enteric coatings for orally administered dosage forms are also contemplated, such as those described in U.S. Pat. No. 4,704,295, “Enteric Film-Coating Compositions,” issued Nov. 3, 1987; U.S. Pat. No. 4,556,552, “Enteric Film-Coating Compositions,” issued Dec. 3, 1985; U.S. Pat. No. 4,309,404, “Sustained Release Pharmaceutical Compositions,” issued Jan. 5, 1982; and U.S. Pat. No. 4,309,406, “Sustained Release Pharmaceutical Compositions,” issued Jan. 5, 1982. As regards formulations for administering a compound of Formula I as disclosed herein, one particularly useful embodiment.

For intravenous injections or drips or infusions, NR4A inhibitor can be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspension, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

In some embodiments of any of the aspects, the NR4A inhibitor is administered as a monotherapy, e.g., another treatment for the clonal hematopoiesis, myelodysplasia, and leukemia is not administered to the subject.

Nucleic acid inhibitor can be formulated into liposomes. Exemplary liposomes can comprise, e.g., DSPC, DPPC, DSPG, Cholesterol, hydrogenated soy phosphatidylcholine, soy phosphatidyl choline, methoxypolyethylene glycol (mPEG-DSPE) phosphatidyl choline (PC), phosphatidyl glycerol (PG), distearoylphosphatidylcholine, and combinations thereof.

### Effective Amount

The phrase “therapeutically-effective amount” as used herein means that amount of a compound, material, or composition which is effective for producing some desired therapeutic effect in at least a sub-population of cells in an animal at a reasonable benefit/risk ratio applicable to any medical treatment. According, a “therapeutically effective amount” refers to an amount effective, at dosage and periods of time necessary, to achieve a desired therapeutic result. A therapeutic result can be, e.g., lessening of symptoms, prolonged survival, improved mobility, and the like. A therapeutic result need not be a “cure.”

Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

Depending on the route of administration, effective doses can be calculated according to the body weight, body surface area, or organ size of the subject to be treated. Optimization of the appropriate dosages can readily be made by one skilled in the art in light of pharmacokinetic data observed in human clinical trials. Alternatively, or additionally, the dosage to be administered can be determined from studies using animal models for the particular type of condition to be treated, and/or from animal or human data obtained from agents which are known to exhibit similar pharmacological activities. The final dosage regimen will be determined by the attending surgeon or physician, considering various factors which modify the action of active agent, e.g., the agent's specific activity, the agent's specific half-life in vivo, the severity of the condition and the responsiveness of the patient, the age, condition, body weight, sex and diet of the patient, the severity of any present infection, time of administration, the use (or not) of other concomitant therapies, and other clinical factors.

Determination of an effective amount is well within the capability of those skilled in the art. Generally, the actual effective amount can vary with the specific compound, the use or application technique, the desired effect, the duration of the effect and side effects, the subject's history, age, condition, sex, as well as the severity and type of the medical condition in the subject, and administration of other pharmaceutically active agents. Accordingly, an effective dose of compound described herein is an amount sufficient to produce at least some desired therapeutic effect in a subject.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of use or administration utilized.

The effective dose can be estimated initially from cell culture assays. A dose can be formulated in animal models to achieve a circulating plasma concentration range that includes the IC50 (i.e., the concentration of the therapeutic which achieves a half-maximal inhibition of symptoms) as determined in cell culture. Levels in plasma can be measured, for example, by high performance liquid chromatography. The effects of any particular dosage can be monitored by a suitable bioassay. The effective plasma concentration for a compound as disclosed herein can be about 0.01 μM to about 10 μM, about 0.2 μM to about 5 μM, or about 0.8 to about 3 μM in a subject, such as a rat, dog, or human.

Generally, the compositions are administered so that a compound described herein is used or given at a dose from 1 μg/kg to 1000 mg/kg; 1 μg/kg to 500 mg/kg; 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. Further contemplated is a dose (either as a bolus or continuous infusion) of about 0.1 mg/kg to about 10 mg/kg, about 0.3 mg/kg to about 5 mg/kg, or 0.5 mg/kg to about 3 mg/kg. It is to be further understood that the ranges intermediate to those given above are also within the scope of this disclosure, for example, in the range 1 mg/kg to 10 mg/kg, for example use or dose ranges such as 2 mg/kg to 8 mg/kg, 3 mg/kg to 7 mg/kg, 4 mg/kg to 6 mg/kg, and the like.

The NR4A inhibitor can be administered at once, or can be divided into a number of smaller doses to be administered at intervals of time. Thus, in some embodiments, the NR4A inhibitor is administered once a day. In some other embodiments, NR4A inhibitor is administered multiple times, e.g., two, three, four, five or more times a day.

It is understood that the precise dosage and duration of treatment will be a function of route of administration, the carrier and other variables that can be determined empirically using known testing protocols or by extrapolation from in vivo or in vitro test data. It is to be noted that concentrations and dosage values can also vary with the age of the individual treated. It is to be further understood that for any particular subject, specific dosage regimens can need to be adjusted over time according to the individual need and the professional judgment of the person administering or supervising the administration of the formulations. Hence, the concentration ranges set forth herein are intended to be exemplary and are not intended to limit the scope or practice of the claimed formulations.

The NR4A inhibitor can be administered as a single bolus or multiple boluses, as a continuous infusion, or a combination thereof. For example, the NR4A inhibitor can be administered as a single bolus initially, and then administered as a continuous infusion following the bolus. The rate of the infusion can be any rate sufficient to maintain effective concentration, for example, to maintain effective plasma concentration. Some contemplated infusion rates include from 1 μg/kg/min to 100 mg/kg/min, or from 1 μg/kg/hr to 1000 mg/kg/hr. Rates of infusion can include 0.2 to 1.5 mg/kg/min, or more specifically 0.25 to 1 mg/kg/min, or even more specifically 0.25 to 0.5 mg/kg/min. It will be appreciated that the rate of infusion can be determined based upon the dose necessary to maintain effective plasma concentration and the rate of elimination of the NR4A inhibitor, such that the compound is administered via infusion at a rate sufficient to safely maintain a sufficient effective plasma concentration of NR4A inhibitor in the bloodstream.

In all embodiments where a sample is obtained or has been obtained or provided, the sample can be sample taken, obtained, or provided via minimally invasive methods and/or involves only a minor intervention. In some embodiments of any of the aspects, a sample is taken, obtained, or provided by one or more of a blood draw or prick, an epidermal or mucus membrane swab, buccal sampling, saliva sample, a epidermal skin sampling technique, and/or collection of a secreted or expelled bodily fluid (e.g., mucus, urine, sweat, etc), fecal sampling, semen/seminal fluid sampling, or clippings (e.g., of hair or nails). In some embodiments of any of the aspects, the sample comprises, consists of, or consists essentially of blood (or any fraction or component thereof), serum, urine, mucus, epithelial cells, saliva, buccal cells, a secreted or expelled bodily fluid, and/or hair or nail clippings.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other than in the operating examples, or where otherwise indicated, all numbers expressing quantities of ingredients or reaction conditions used herein should be understood as modified in all instances by the term “about.” The term “about” when used to described the present invention, in connection with percentages means ±1%, +1.5%, +2%, +2.5%, ±3%, +3.5%, ±4%, ±4.5%, or +5%.

The singular terms “a,” “an,” and “the” include plural referents unless context clearly indicates otherwise. Similarly, the word “or” is intended to include “and” unless the context clearly indicates otherwise.

As used herein the terms “comprising” or “comprises” means “including” or “includes” and are used in reference to compositions, methods, systems, and respective component(s) thereof, that are useful to the invention, yet open to the inclusion of unspecified elements, whether useful or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, systems, and respective components thereof as described herein, which are exclusive of any element not recited in that description of the embodiment.

The abbreviation, “e.g.” is derived from the Latin exempli gratia, and is used herein to indicate a non-limiting example. Thus, the abbreviation “e.g.” is synonymous with the term “for example.”

The terms “significantly different than,”, “statistically significant,” and similar phrases refer to comparisons between data or other measurements, wherein the differences between two compared data or other measurements are evidently or reasonably different to the trained observer, or statistically significant (if the phrase includes the term “statistically” or if there is some indication of statistical test, such as a p-value, or if the data, when analyzed, produce a statistical difference by standard statistical tests known in the art).

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The term “a reference level” as used herein refer to a negative control. For example, in the context of treatment, a reference level is the level if a subject is not treated. In some embodiments, a reference level in the context of diagnosis is the level present in a normal healthy subject. The term “normal healthy subject” refers to a subject who has no symptoms of any diseases or disorders, or who is not identified with any diseases or disorders, or who is not on any medication treatment, or a subject who is identified as healthy by physicians based on medical examinations. In some embodiments, a reference level used herein refers to the level measured prior to onset of treatment.

As used herein, the terms “treat,” “treatment,” “treating,” or “amelioration” are used herein to characterize a method or process that is aimed at (1) delaying or preventing the onset of a disease or condition; (2) slowing down or stopping the progression, aggravation, or deterioration of the symptoms of the disease or condition; (3) bringing about ameliorations of the symptoms of the disease or condition; or (4) curing the disease or condition. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased morbidity or mortality. The term “treatment” of a disease also includes providing relief from the symptoms or side-effects of the disease (including palliative treatment).

A treatment can be administered prior to the onset of the disease, for a prophylactic or preventive action. Alternatively, or additionally, the treatment can be administered after initiation of the disease or condition, for a therapeutic action. Accordingly, in some embodiments of any of the aspects, described herein is a prophylactic method of treatment. As used herein “prophylactic” refers to the timing and intent of a treatment relative to a disease or symptom, that is, the treatment is administered prior to clinical detection or diagnosis of that particular disease or symptom in order to protect the patient from the disease or symptom. Prophylactic treatment can encompass a reduction in the severity or speed of onset of the disease or symptom, or contribute to faster recovery from the disease or symptom. Accordingly, the methods described herein can be prophylactic relative to clonal hematopoiesis, myelodysplasia, and leukemia. In some embodiments of any of the aspects, prophylactic treatment is not prevention of all symptoms or signs of a disease.

In some embodiments, treatment is therapeutic and does not include prophylactic treatment.

As used herein, “alleviating” a symptom of a clonal hematopoiesis, myelodysplasia, and leukemia is ameliorating any condition or symptom associated with the clonal hematopoiesis, myelodysplasia, and leukemia. As compared with an equivalent untreated control, such reduction is by at least 5%, 10%, 20%, 40%, 50%, 60%, 80%, 90%, 95%, 99% or more as measured by any standard technique.

As used herein, “inhibitor” refers to a molecule or compound which can decrease the expression and/or activity of a target, e.g. by at least 10% or more, e.g. by 10% or more, 50% or more, 70% or more, 80% or more, 90% or more, 95% or more, or 98% or more. The efficacy of an inhibitor of one or more targets, e.g. its ability to decrease the level and/or activity of the target can be determined, e.g. by measuring the level of an expression product of the target and/or the activity of the target. In some embodiments of any of the aspects, the inhibitor can be an inhibitory nucleic acid; an aptamer; an antibody reagent; an antibody; or a small molecule. An inhibitor of a target described herein can inhibit the activity, expression, or accumulation of the target polypeptide. Inhibitors can include inhibitors that act directly on the target itself (e.g., that bind to the protein or transcript, e.g., direct inhibitors).

As used herein, the term “subject” refers to any living organism which can be administered compound and/or pharmaceutical compositions of the present invention. The term includes, but is not limited to, humans, non-human primates such as chimpanzees and other apes and monkey species; farm animals such as cattle, sheep, pigs, goats and horses, domestic subjects such as dogs and cats, laboratory animals including rodents such as mice, rats and guinea pigs, and the like. The term does not denote a particular age or sex. Thus, adult, child and newborn subjects, whether male or female, are intended to be covered. The term “subject” is also intended to include living organisms susceptible to conditions or disease states as generally disclosed, but not limited to, throughout this specification. Examples of subjects include humans, dogs, cats, cows, goats, and mice. The term subject is further intended to include transgenic species. The term “subject” and “individual” are used interchangeably herein, and refer to an animal, for example a human or non-human mammals/animals, to whom treatment, including prophylactic treatment, with the compounds and compositions according to the present invention, is provided. The term “non-human animals” and “non-human mammals” are used interchangeably herein and include all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc.

In some embodiments, the subject is a human or animal. Usually the animal is a vertebrate such as a primate, rodent, domestic animal or game animal. Primates include chimpanzees, cynomolgous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used as subjects that represent animal models of a fibrotic disease or disorder.

It is noted that a human subject can be of any age, gender, race or ethnic group, e.g., Caucasian (white), Asian, African, black, African American, African European, Hispanic, Middle eastern, etc.

In addition, the methods described herein can be used to treat domesticated animals and/or pets. A subject can be male or female. A subject can be one who has been previously diagnosed with or identified as suffering from or having a disease or disorder needing treatment, but need not have already undergone treatment. For example, the subject can be one who has been previously diagnosed with or identified as suffering from or having a microbial infection, e.g., a fungal infection.

In some embodiments of any one of the aspects, the subject is human.

A subject can be one who has been previously diagnosed with or identified as suffering from or having a condition in need of treatment (e.g. clonal hematopoiesis, myelodysplasia, and leukemia) or one or more complications related to such a condition, and optionally, have already undergone treatment for clonal hematopoiesis, myelodysplasia, and leukemia or the one or more complications related to clonal hematopoiesis, myelodysplasia, and leukemia. Alternatively, a subject can also be one who has not been previously diagnosed as having clonal hematopoiesis, myelodysplasia, and leukemia or one or more complications related to clonal hematopoiesis, myelodysplasia, and leukemia. For example, a subject can be one who exhibits one or more risk factors for clonal hematopoiesis, myelodysplasia, and leukemia or one or more complications related to clonal hematopoiesis, myelodysplasia, and leukemia or a subject who does not exhibit risk factors.

A “subject in need” of treatment for a particular condition can be a subject having that condition, diagnosed as having that condition, or at risk of developing that condition.

The term “disorder” or “disease” used interchangeably herein, refers to any alteration in the state of the body or of some of its organs, interrupting or disturbing the performance of the functions and/or causing symptoms such as discomfort, dysfunction, distress, or even death to the person afflicted or those in contact with the person. A disease or disorder can also relate to distemper, ailing, ailment, malady, disorder, sickness, illness, complaint, indisposition, affection.

A “somatic mutation,” as used herein, refers to a change in the genetic structure of a subject that is not inherited from a parent, and also not passed to offspring. Hence, a somatic mutation is a genetic change that occurs in any cell after the first cell division, wherein the mutation is replicated in all cells that descend from the mutated cell. The somatic cells that descend from the original mutated cell comprise a clonal variant within the body of the subject. Where these mutations are present in cells of somatic origin and not present in the germline, they are often called a somatic cell mutation or an acquired mutation. Somatic mutations will be present in only a subset of the cells contributing DNA to an analysis, since the mutant sequence will be present in fewer than 50% of the sequence reads arising from that genomic site. In other words, somatic mutations are identified as when a specific sequence is measured as occurring at a fraction of total sequences that deviates significantly from the frequency expected for the far-larger number of inherited variants—namely around 0%, around 50% or around 100%.

Somatic mutations can occur in a sub-population of cells for example, such as a sub-population of hematopoietic cells. Somatic mutations in HSPC genes, such as ASM-1, DNMT3A, IDH1, IDH2, relevant to the composition and methods described herein include any nucleic acid or consequent amino acid somatic mutations in HSPC genes, such as ASM-1, DNMT3A, IDH1, IDH2, found in a subset of hematopoietic cells. Such somatic mutations in HSPC genes, such as ASXL1, DNMT3A, IDH1, IDH2, can be disruptive, in that they have an observed or predicted effect on protein function, or non-disruptive. As used herein, a “non-disruptive mutation” is typically a missense mutation, in which a codon is altered such that it codes for a different amino acid, but the encoded protein, i.e., ASXL1, DNMT3A, IDH1, IDH2, is still expressed. Somatic mutations in HSPC genes, such as ASXL1, DNMT3A, IDH1, IDH2, include, for example, frameshift mutations, nonsense mutations, missense mutations or splice-site variant mutations, as those terms are known to those of ordinary skill in the art.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., NR4A1, NR4A2, or NR4A3 genomic DNA or cDNA. Suitable RNA can include, e.g., NR4A1, NR4A2, or NR4A3 mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Marker” in the context of the present invention refers to an expression product, e.g., nucleic acid or polypeptide which is differentially present in a sample taken from subjects having having clonal hematopoiesis, myelodysplasia, and leukemia, as compared to a comparable sample taken from control subjects (e.g., a healthy subject). The term “biomarker” is used interchangeably with the term “marker.”

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

The term “statistically significant” or “significantly” refers to statistical significance and generally means a two standard deviation (2SD) or greater difference.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

The technology may be as described in any one of the following numbered paragraphs:

Paragraph 1: A method for treating a clonal hematopoietic disease or disorder, the method comprising administering an inhibitor of a member of the nuclear receptor 4A (NR4A) family.

Paragraph 2: The method of paragraph 1, wherein the inhibitor binds with the NR4A family member or with a nucleic acid encoding the NR4A family member.

Paragraph 3: The method of paragraph 1 or 2, wherein the NR4A family member is selected from the group consisting of NR4A1, NR4A2 and NR4A3.

Paragraph 4: The method of any one of paragraphs 1-3, wherein the NR4A family member is NR4A1.

Paragraph 5: The method of any one of paragraphs 1-4, wherein the inhibitor is a nucleic acid.

Paragraph 6: The method of paragraph 5, wherein the nucleic acid is selected from the group consisting of siRNAs, antisense oligonucleotides, aptamers, ribozymes, and triplex forming oligonucleotides.

Paragraph 7: The method of paragraphs 5 or 6, wherein the inhibitor comprises a nucleotide sequence substantially complementary to at least a portion of a nucleic acid encoding the NR4A family member.

Paragraph 8: The method of any one of paragraphs 5-7, wherein the inhibitor comprises a nucleotide sequence substantially complementary to at least 15 contiguous nucleotides of SEQ ID NO: 1 (NR4A1 sequence), SEQ ID NO: 6 (NR4A2 sequence) or SEQ ID NO: 9 (NR4A3 sequence).

Paragraph 9: The method of any one of paragraphs 1-4, wherein the inhibitor is an antibody or antigen binding fragment thereof that binds the NR4A family member.

Paragraph 10: The method of paragraph 9, wherein the antibody is an anti-NR4A1 antibody, an anti-NR4A2 antibody, an anti-NR4A3 antibody, or antigen binding fragment thereof

Paragraph 11: The method of paragraph 9 or 10, wherein the antibody is a monoclonal antibody.

Paragraph 12: The method of any one of paragraphs 9-11, wherein the antibody is a humanized antibody.

Paragraph 13: The method of any one of paragraphs 1-4, wherein the inhibitor is a small molecule.

Paragraph 14: The method of paragraph 13, wherein the inhibitor is a 1,1-bis(3′-indolyl)-1-(p-substituted phenyl)methane (C-DIM) compound.

Paragraph 15: The method of paragraph 13 or 14, wherein the inhibitor is 1,1-bis(3′-indolyl)-1-(p-hydroxyphenyl)methane (DIM-C-pPhOH), 1,1-bis(3′-indolyl)-1-(3-chloro-4-hydroxy-5-methoxyphenyl)methane (DIM-C-pPhOh-3-Cl-5-OCH₃), 1,1-bis(3′-indolyl)-1-(3,5-dibromo-4-hydroxyphenyl)methane (DIM-C-pPhOH-3,5-Br2), camptothecin (CPT), or a cyclooxygenase (COX)-2 inhibitor (celecoxib analogue SC-236).

Paragraph 16: The method of any one of paragraphs 1-15, wherein the clonal hematopoietic disease or disorder is clonal hematopoiesis, myelodysplastic syndromes (MDS) or leukemia.

Paragraph 17: The method of any one of paragraphs 1-16, wherein the clonal hematopoietic disease or disorder is leukemia.

Paragraph 18: The method of paragraph 17, wherein the leukemia is acute myelogenous leukemia (AML) or chronic myeloid leukemia (CVL).

Paragraph 19: The method of any one of paragraphs 1-16, the clonal hematopoietic disease or disorder is MDS.

Paragraph 20: The method of paragraph 19, wherein the MDS is MDS with multilineage dysplasia (MDS-MLD), MDS with single lineage dysplasia (MDS-SLD), MDA with ring sideroblasts (MDS-RS), MDS with excess blasts (MDS-EB), MDS with isolated del(5q), and/or MDS unclassifiable (MDS-U).

Paragraph 21: The method of any one of paragraphs 1-20, further comprising co-administering an immunomodulatory agent to the subject.

Paragraph 22: The method of any one of paragraphs 1-20, further comprising co-administering an anti-inflammatory agent to the subject.

Paragraph 23: The method of any one of paragraphs 1-20, wherein the subject comprises at least one mutation in a nucleic acid encoding ASXL transcriptional regulator 1 (ASXL1), DNA (cytosine-5)-methyltransferase 3A (DNMT3A), isocitrate dehydrogenase (NADP(+)) 1(IDH1) or isocitrate dehydrogenase (NADP(+)) 2 (IDH 2).

Paragraph 24: The method of paragraph 16, further comprising, prior to onset of the treatment regime, identifying the subject with at least one mutation in a nucleic acid encoding ASXL1, DNMT3A, IDH1 or IDH 2.

Paragraph 25: The method of any one of paragraphs 1-24, further comprising a step of assaying a sample from the subject for at least one mutation in a nucleic acid encoding ASXL1, DNMT3A, IDH1 or IDH 2 prior to onset of the treatment regime.

Paragraph 26: The method of any one of paragraphs 1-25, wherein the subject is a mammal.

Paragraph 27: The method of any one of paragraphs 1-26, wherein the subject is human.

### Examples

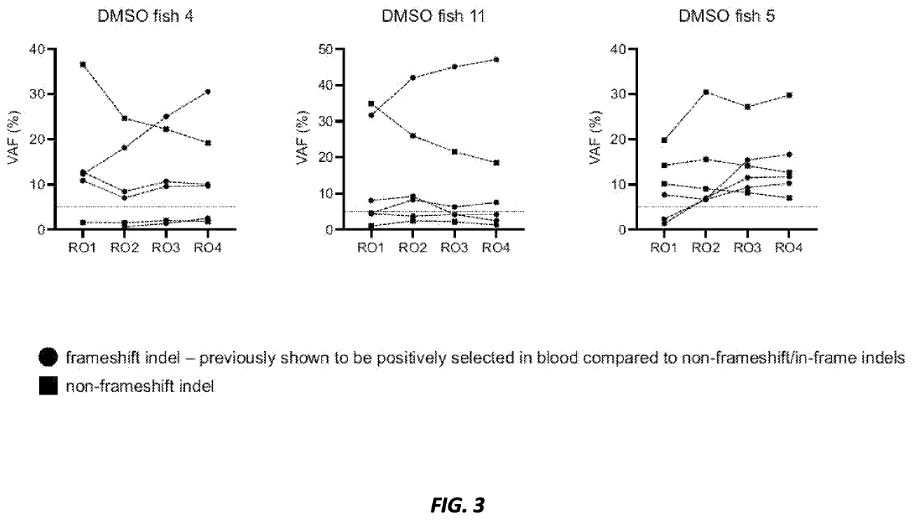

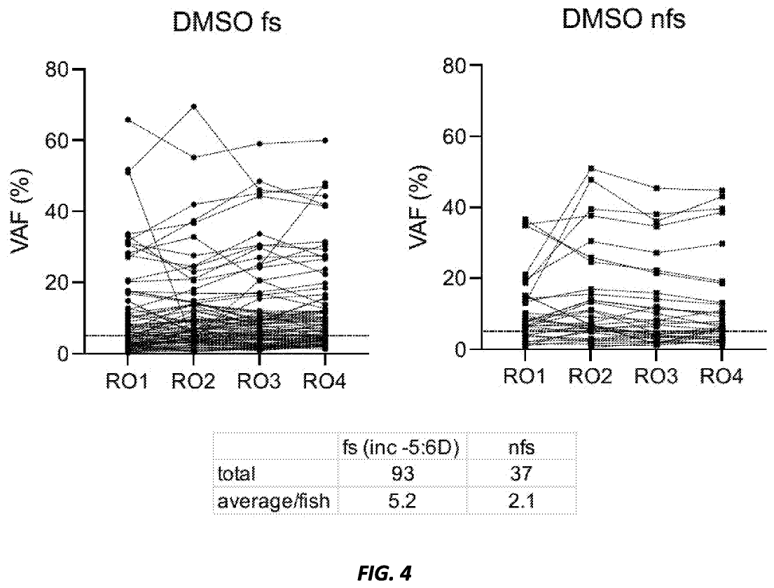

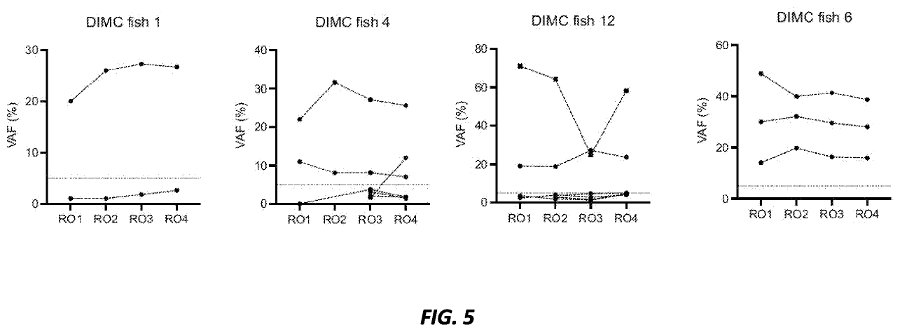

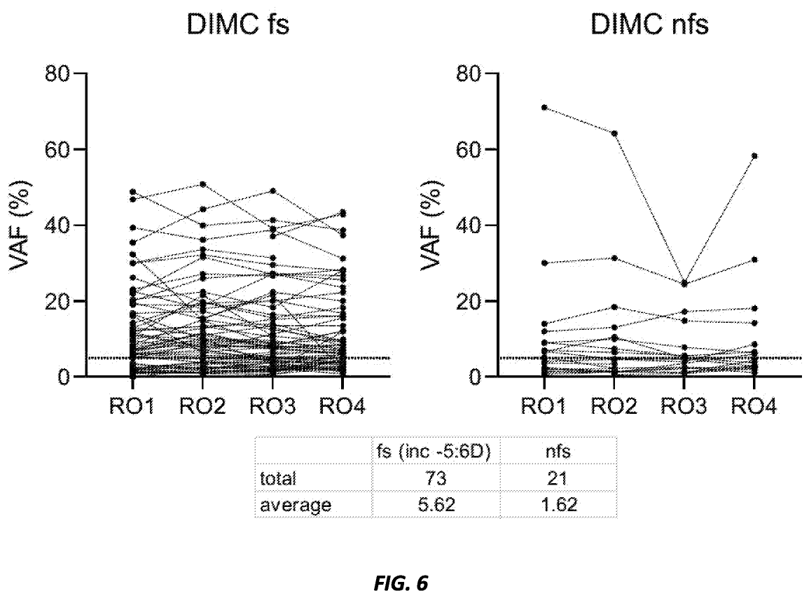

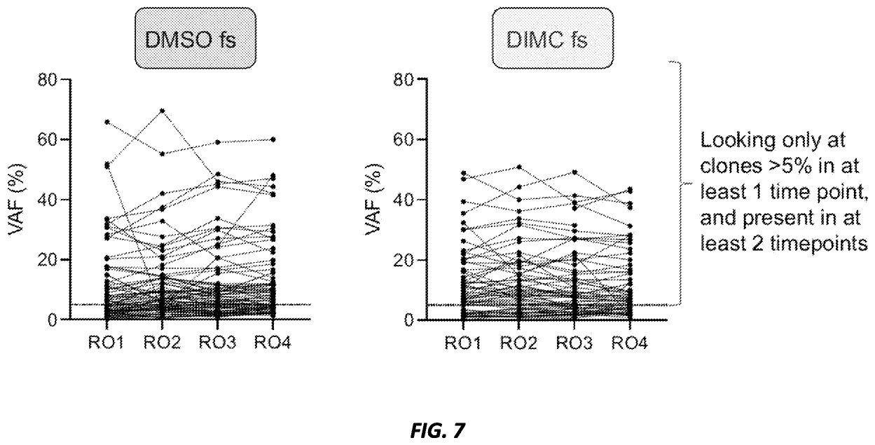

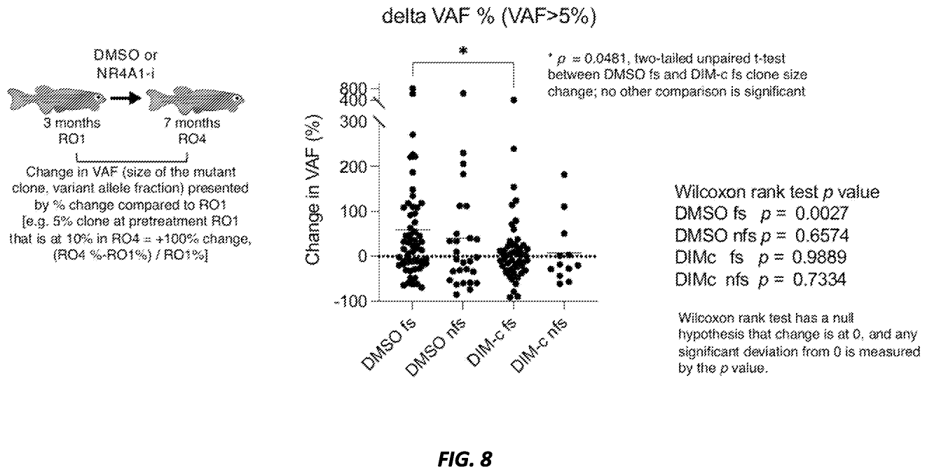

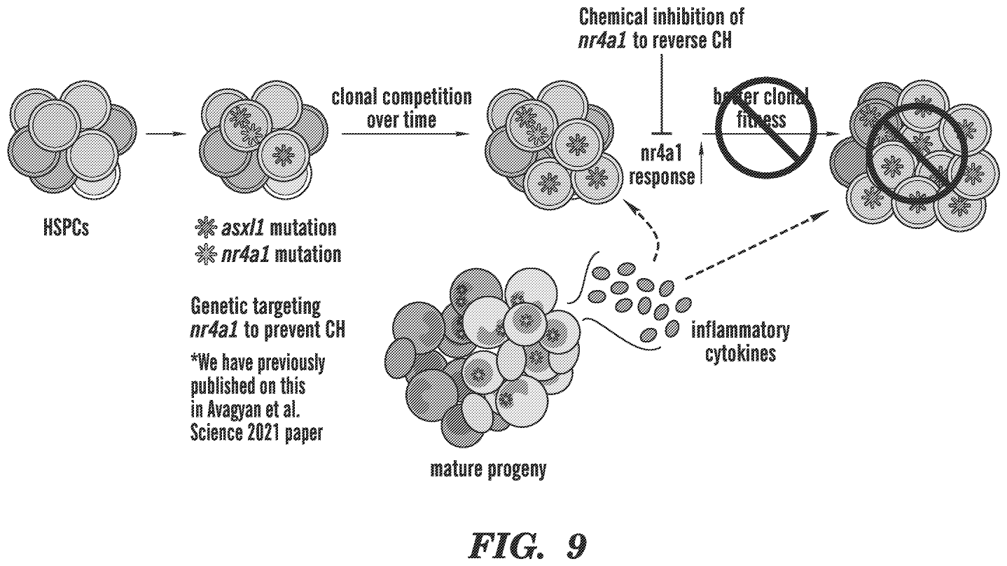

Clonal hematopoiesis. Zebrafish were generated with mosaic mutations in asxl1 using the TWISTR method (Avagyan et a1 Science 2021, PMID 34735227). Briefly, they were injected with guide-RNAs targeting asxl1 gene with Cas9 mRNA into 1-cell embryos of zebrafish, and grown to adulthood. At 3 months retroortibital bleeding was performed on the adult zebrafish and the size of the mutant clones in the peripheral blood was measured by sequencing the genomic DNA at the targeting locus of asxl1. Zebrafish with at least 1 mutant allele of 5% of greater allele size were chosen for the cohorts, randomly assigned to be treated with either DMSO or NR4A1 inhibitor (DIM-C). Zebrafish with mosaic mutations in asxl1 were treated with either DMSO or an NR4A1 inhibitor (DIM-C) and analyzed over a three-month period. Peripheral blood was collected every month to determine the size of the mutant clones over time. The results are shown in FIGS. 3-8.

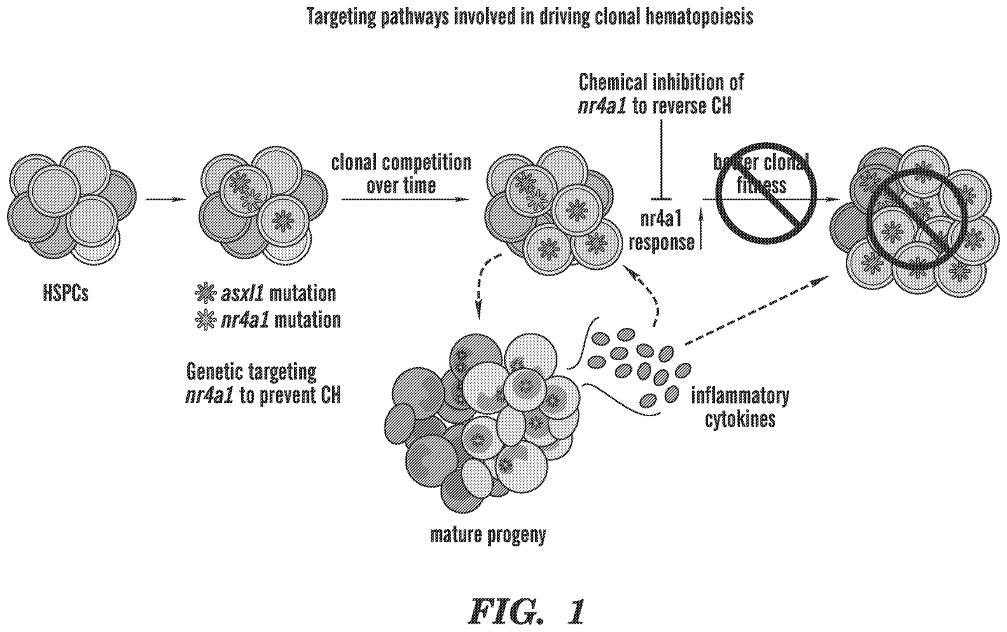

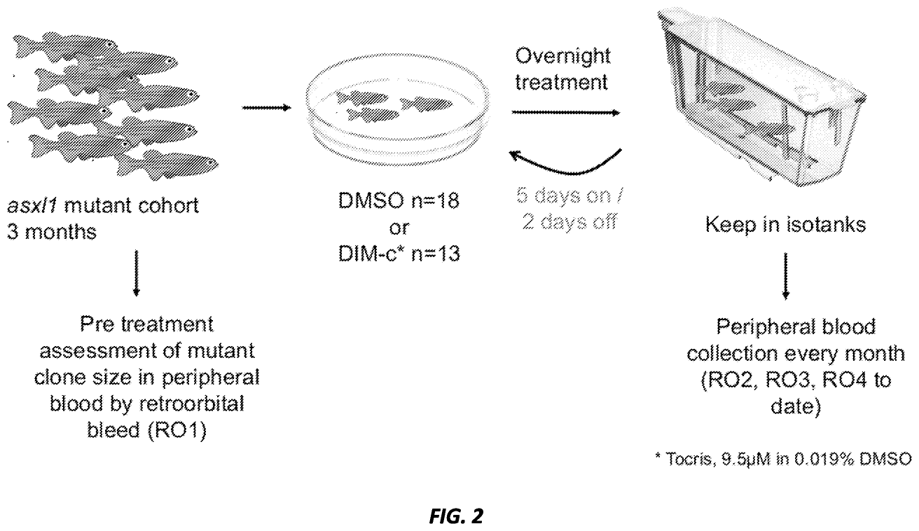

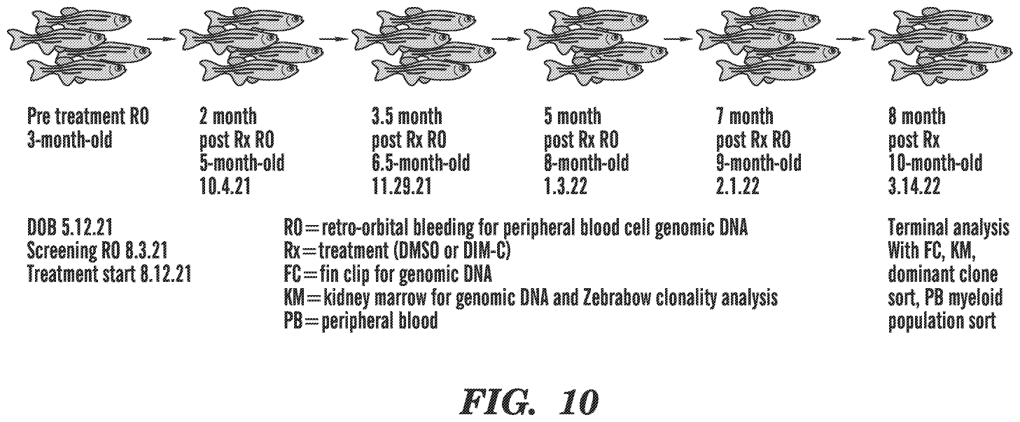

It was previously shown by early genetic targeting nr4a1 during development prevented clonal growth of mutant cells. Next, the hypothesis that pharmacologic inhibition of nr4a1 would halt or reduce mutant clonal growth over time was tested (FIG. 1). To test this hypothesis, a cohort of asxl1 mutant zebrafish were generated and screened for the presence of asxl1 mutant clone at at least 5% VAF in peripheral blood. The positively screened mutant fish were then treated either with DMSO or minimally toxic dose of DIM-c (Tocris compound) at 9.5 uM concentration overnight, between 14-16 hours long, 5 days of the week for 6 months. Peripheral blood from both groups was collected by retroorbital bleeding every month for measurement of the mutant clones in the blood by next generation sequencing (FIG. 2). N=are the numbers of fish at the initiation of treatment. A more detailed experimental schema with exact dates in order to calculate exact ages of the fish or post treatment timepoints is shown in FIG. 10.

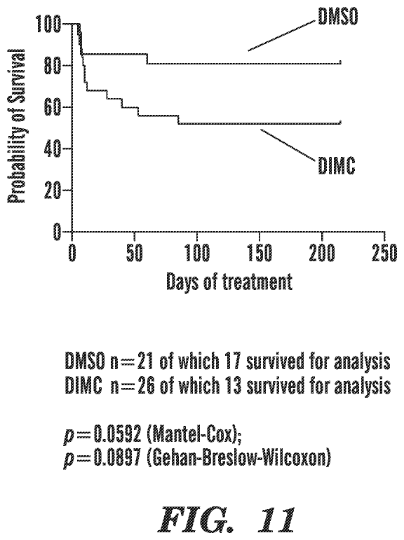

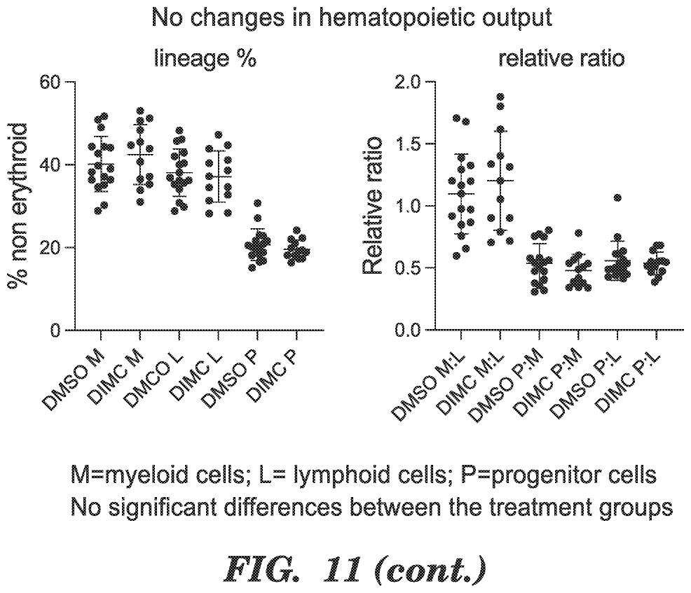

FIG. 11 examines the survival curve of the fish in two cohorts. An event was finding the fish dead or moribund after overnight treatment; no fish died during the day or off treatment days or as a consequence of retroorbital bleeding. No statistical difference was found with p values >0.05 by two different tests between the groups. 17 DMSO and 13 DIMC fish were left for final analysis. Additionally, marrow cellular composition was analyzed at 8 months post treatment by flow cytometry. There was no difference between the treatment groups in the three major lineages analyzed—myelomonocytes, lymphocytes and progenitor/precursors.

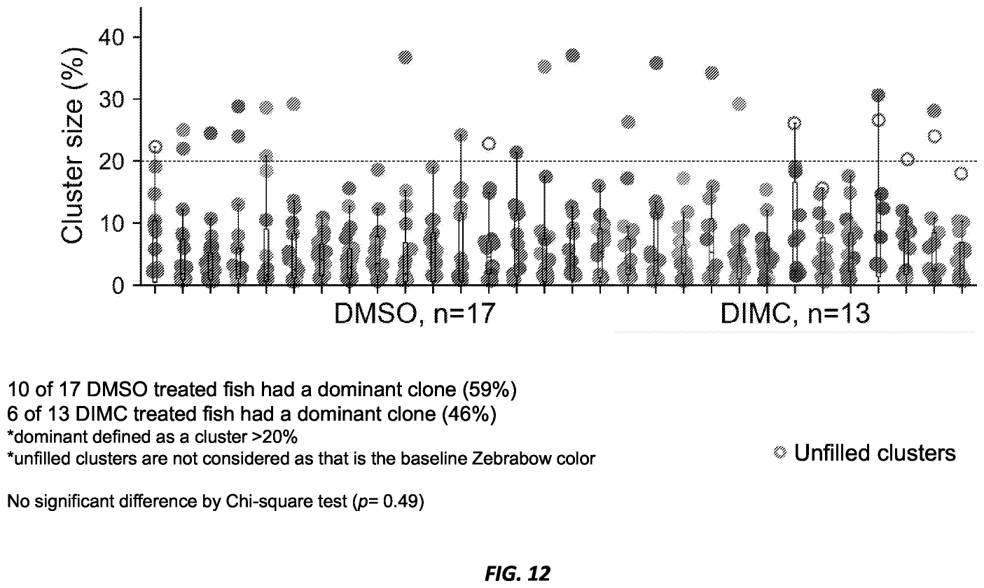

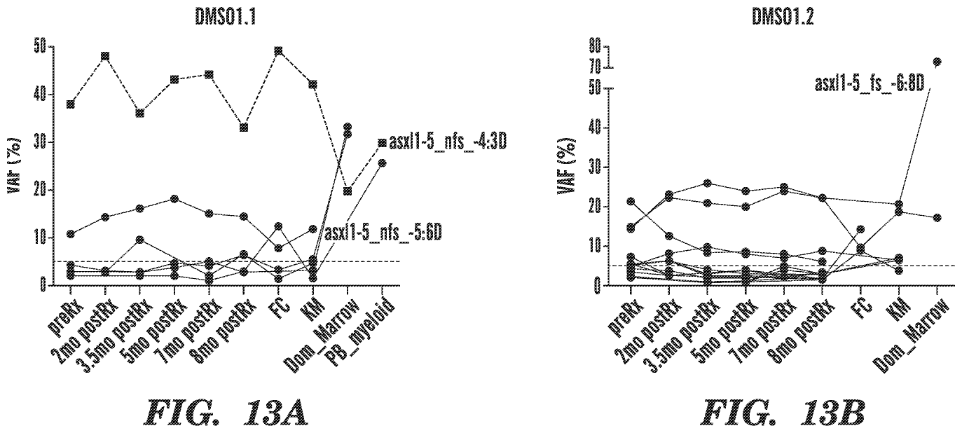

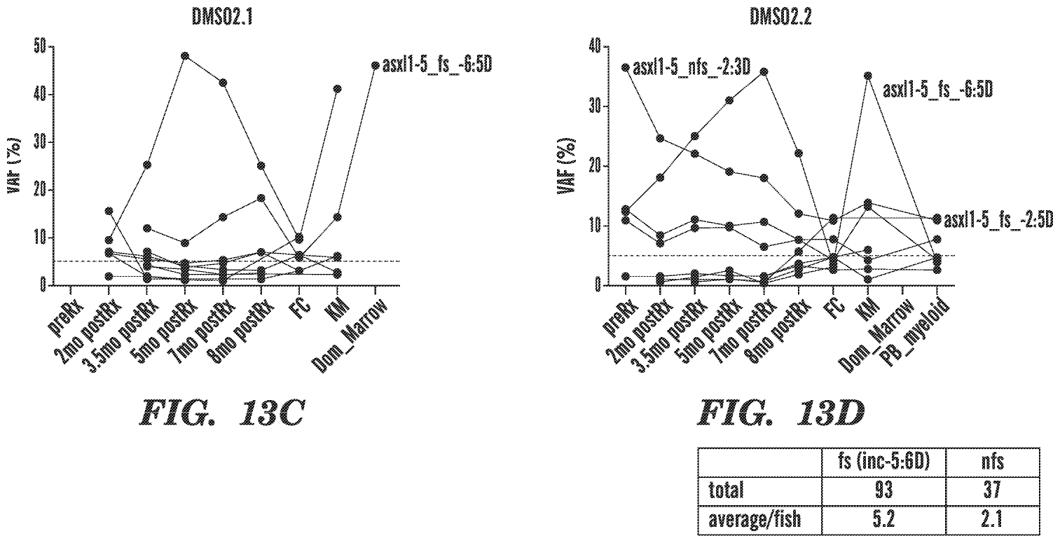

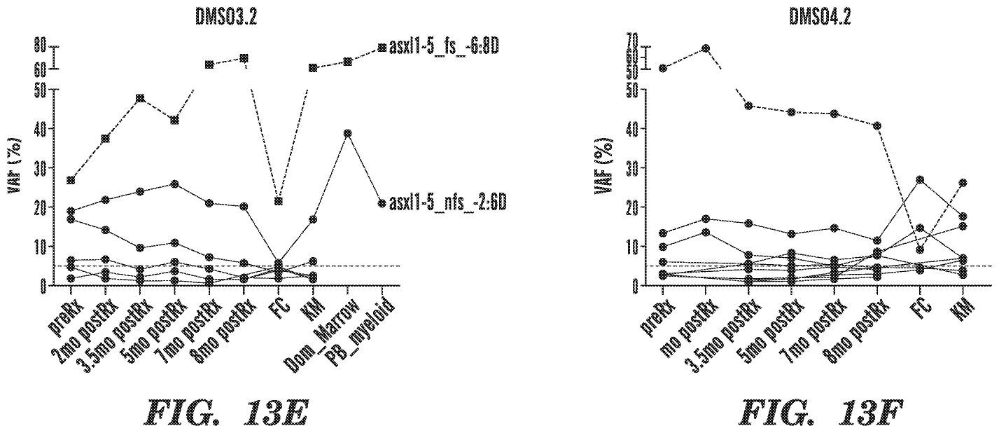

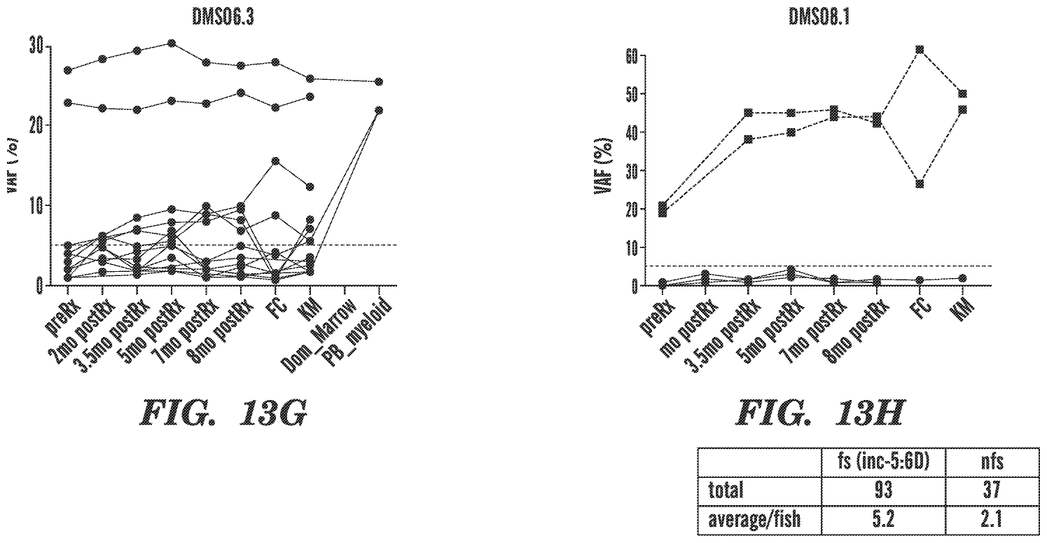

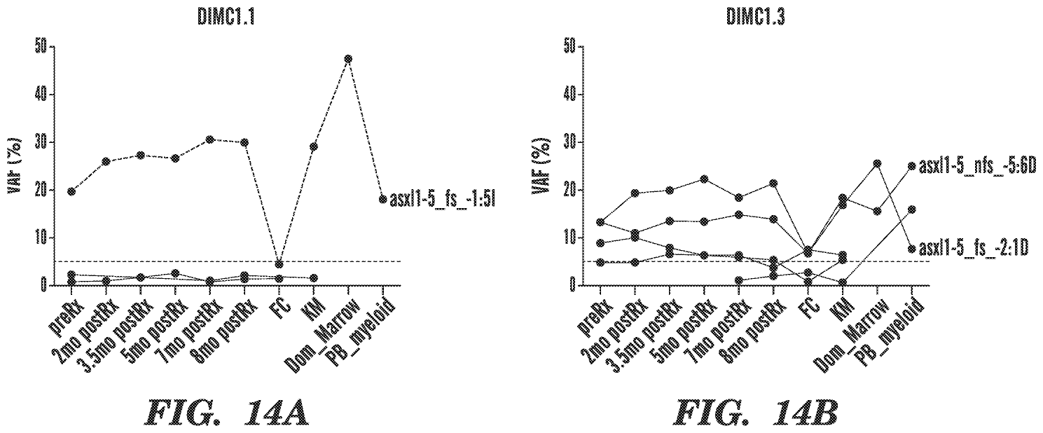

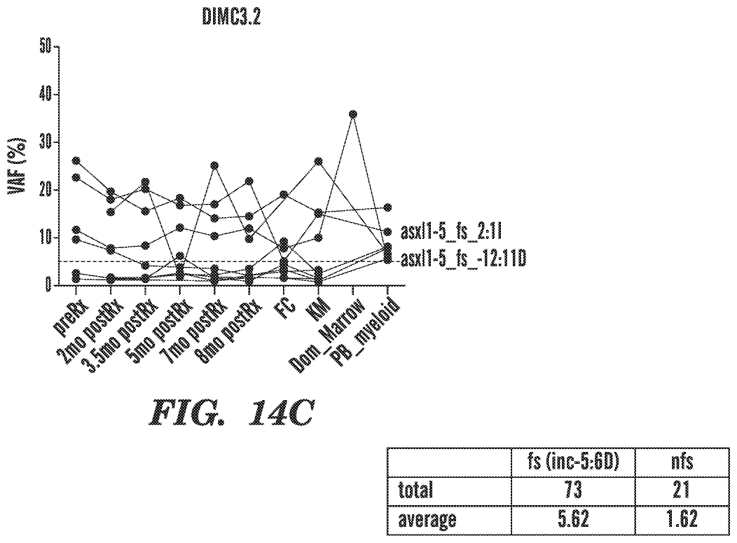

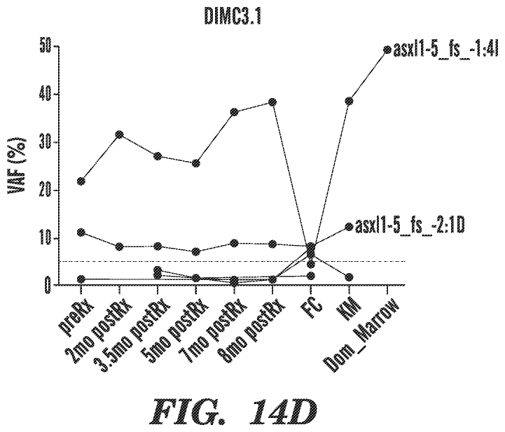

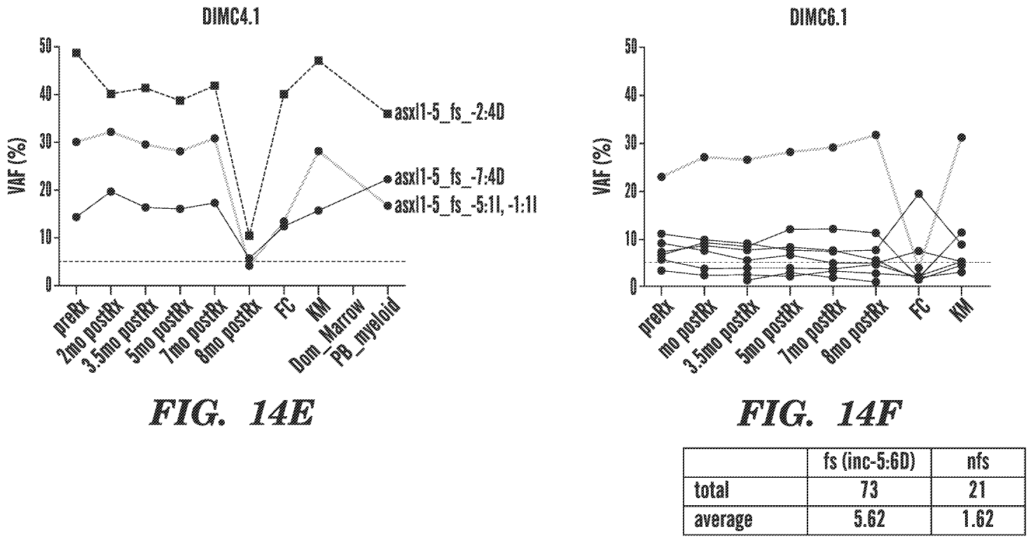

FIG. 12 analyzes the clonal composition by Zebrabow color-labeling in marrow myeloid cells. The data did not show any major difference between treatment groups for loss or gain of dominant clonal status of >20% clone size.

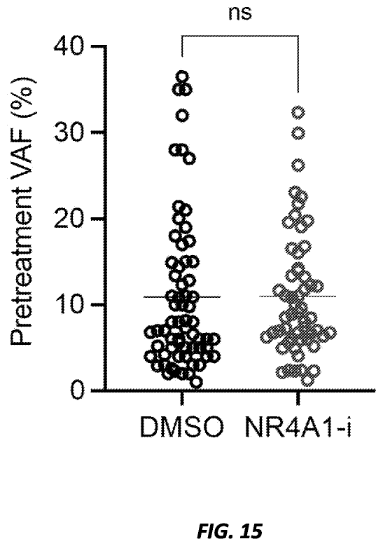

FIG. 15 examines no difference in the size of asxl1 mutant clones pre-treatment in the two treatment arms.

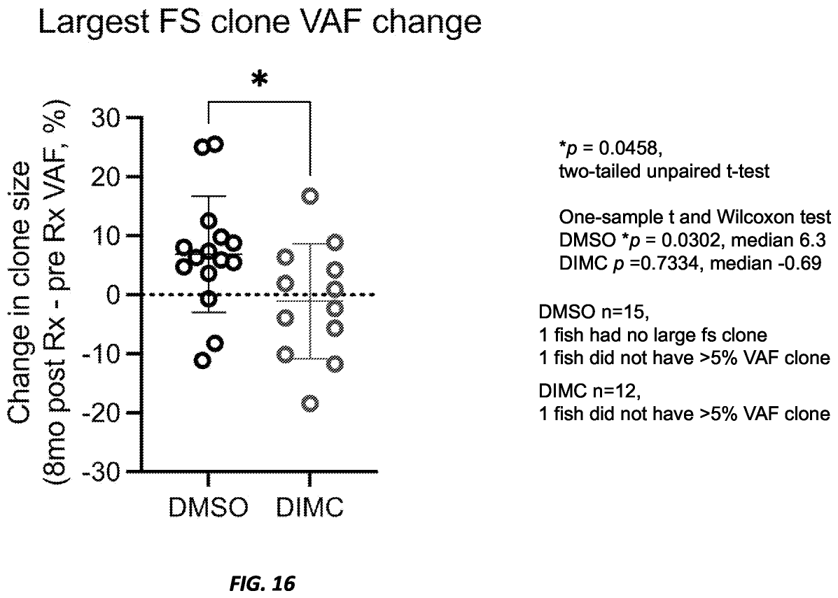

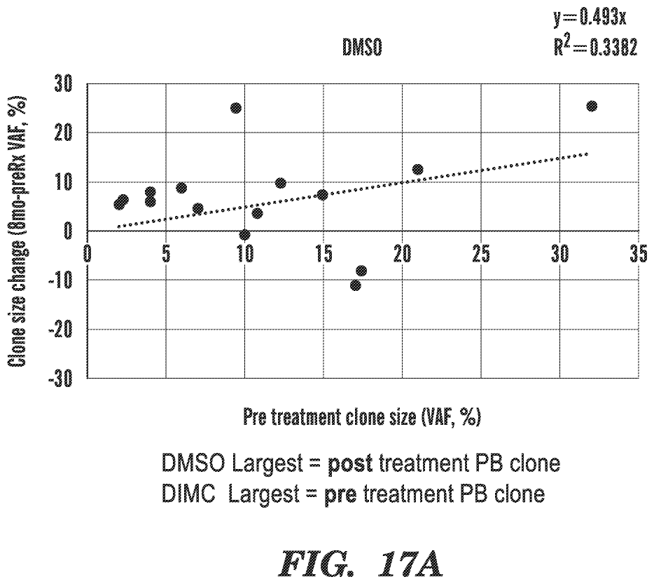

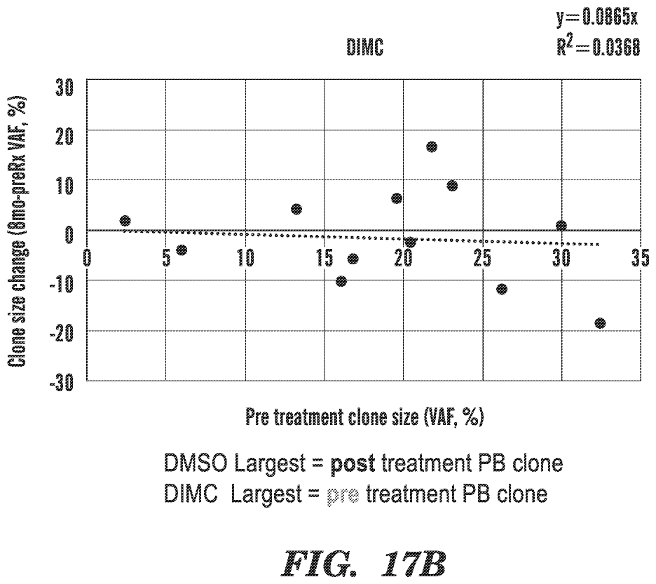

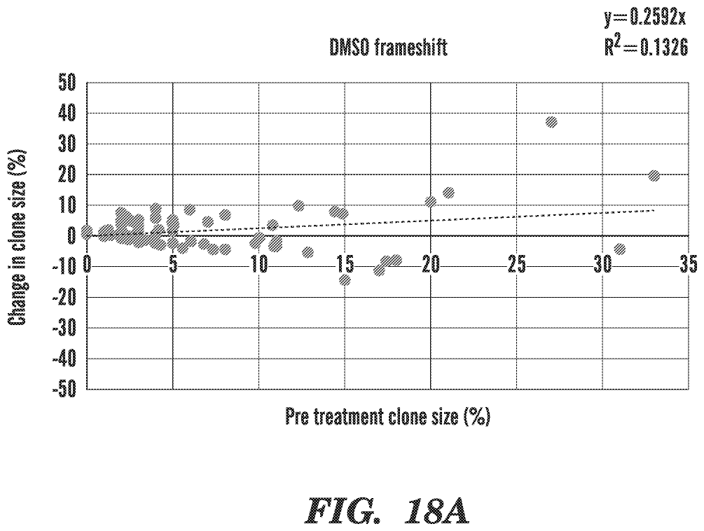

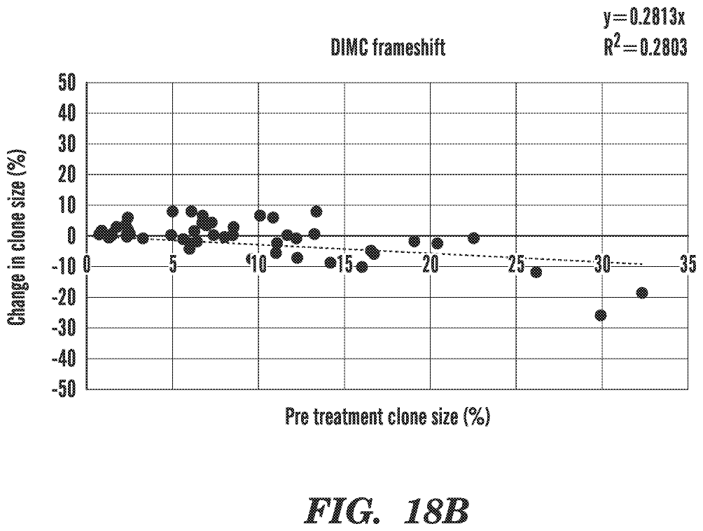

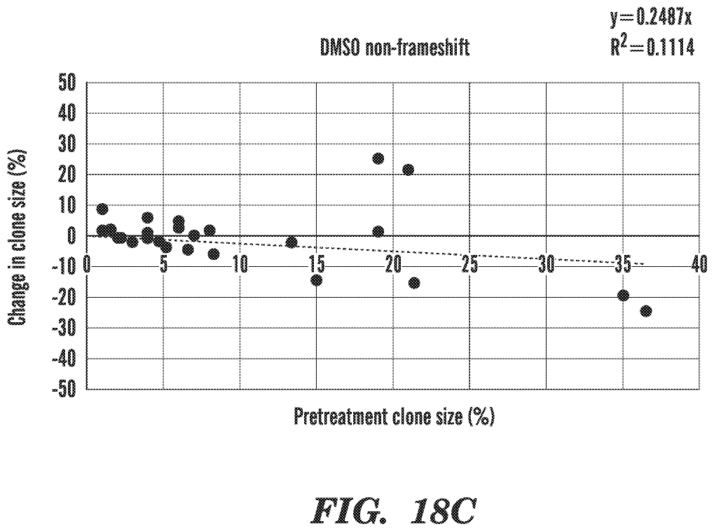

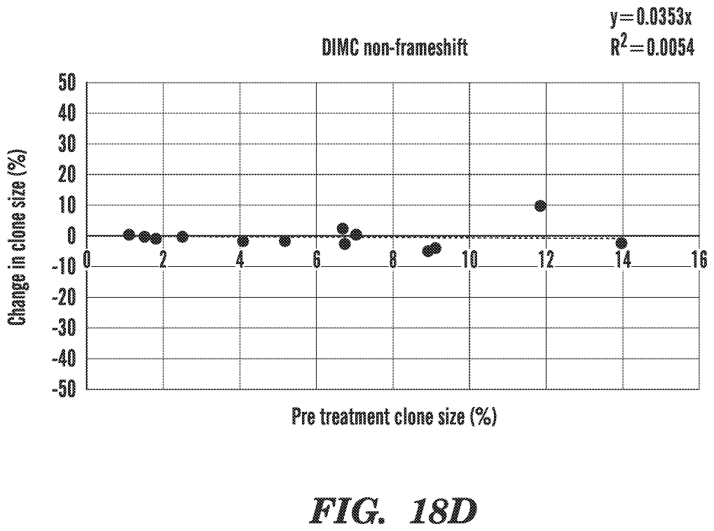

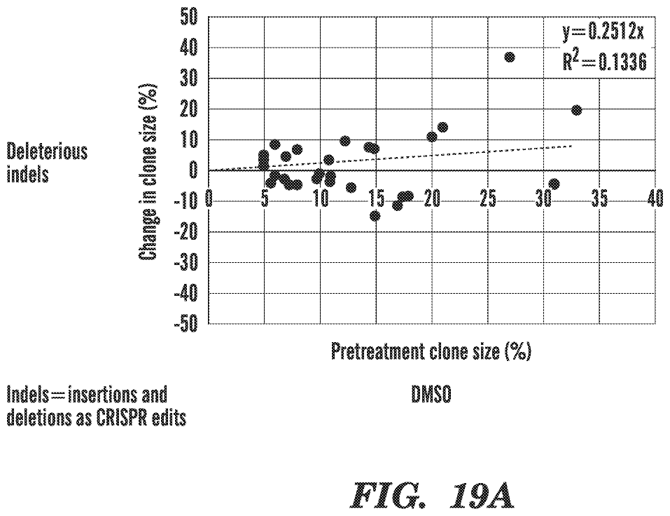

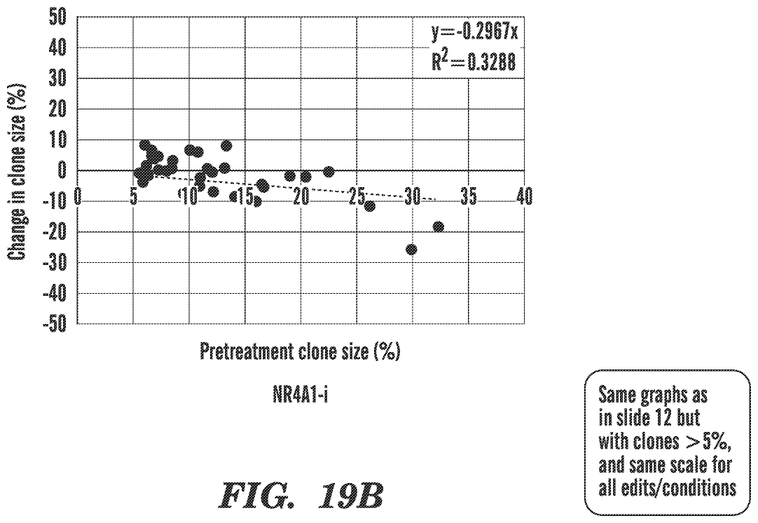

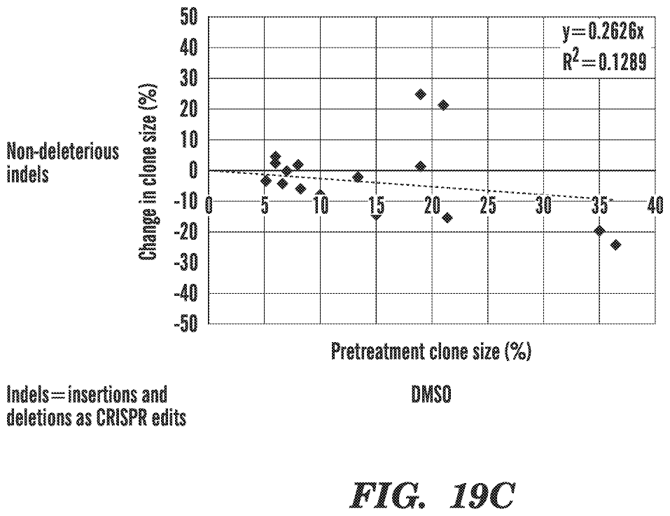

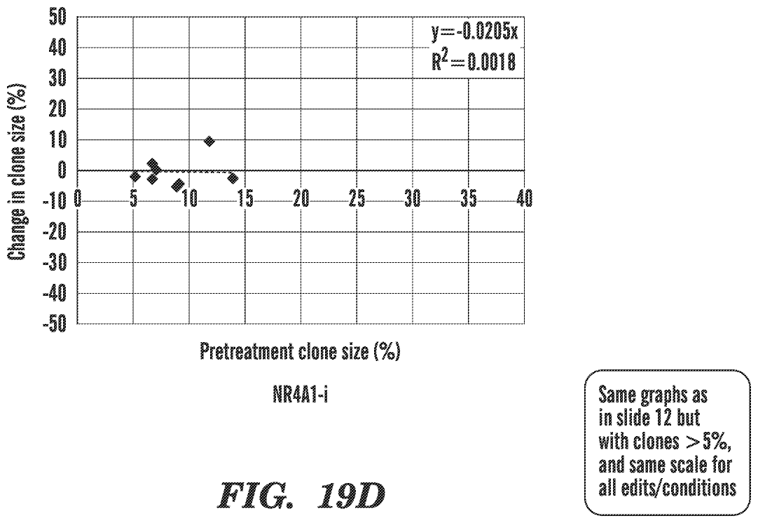

FIG. 16 analyzes how the clone size changes with treatment. In the DMSO treatment group, the largest clone at the last assessment of peripheral blood showed a significant increase of their size compared to the 0 which was the null hypothesis (no change over time). This was shown by the one sample t and Wilcoxon test, p value=0.03. The largest clone if DIMC treatment treatment arm at the first blood sampling had not changed significantly over the same period of time, p value=0.7334. FIGS. 17A-17B, FIGS. 18A-18D, and FIGS. 19A-19B examine the clone sizes in greater detail.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Sequences:

SEQ ID NO: 1 (NC_000012.12:52022832-52059507 Homo sapiens chromosome 12,
GRCh38.p13 Primary Assembly, NR4A1 genomic sequence):
TTCCTGGTGTAAGCTTTGGTATGGATGGTGGCCGTCTCCCTACAGACTGGGAGCTGTTAGAGGGCAGGGA
TCCTAGCTGACACATCTATGTCCTCGCCTTGGTTGGAGGTAGGTATGGGTGGCGGCGAGGGAGGAGATGG
GGGTGGGGCTCAACACACTGTCCAGCCCCTGTCGTCTGGCTGGGTATCGCCAGTGCTCAGGGGCGAGCGG
AATGGAGAGGAGGCGGGGCTGGGGCGCACAGCCGGCAGGGGATCCCTGCCGGAGTCTGCTTTTCACGGGT
TCCCGTCTAGACCAGAGTTGGGGCTCAGCTTGGAGAGATGGGACAGGTGGAGGTGCTGGGGGTTGGAGCC
CCACGCGCTGCTCAAGAGGCTGCAGGCTGGCCTTCAAGGCCTGGGCTAGACTGCGGCGGTTGGGGGCTGG
CGGTGCGGCTCGGGTGGAGTCCGCCGGGCACCGCCCTCCCTCTCCACCCTCGCCCACCCCGGGGCCGCGA
GGCTGTGGGTTGAGCTGCAGAGGCGAGCTCCCTCCTGGGCGTGCCGCCGCCGCGAGTGGGGTGGGAGCGC
AGAGGGGGCCTCGGGGTTATGCTTTGCTGGGATCTGGGAAGGAAGCAGGTGGCAGCGCCGGAGTGAGTAA
CCGCCTCCCGAGGCAGCTAGTCCCGCCGCCTCAGGAAGTCCTGGCGCCGTAGCGCACCGCCGCCCGCCAC
ACCGGGACCTTCCCCTCGCACTAGCCCGCCCCCACCCCGCCTCCATCTCGCGCTCCCAGCCGCAGACACG
GGCGCCTCTGCACTCTCAACCCCTCACCCACGCAGACCCTGGCCCGCCGGTGACCTCCCCTGCGGCTCCT
CCTTCTGCCCCGGCCCAGCCCCTCCCCTGCTGCTCCTCCTCCTGCCCCGGCCCAGCCCGTCCACCGTCCT
CTCATCCCCATGGCCTCGAACCCCAGCCTGCCGAGCCGCTCCTCACAGGGTGCTGTGGCTCCCCGCTGCA
AAGGGGCGGGTCCTGGTAGGGGGCGTGGCCTGGCCGACTTGCGGGGCGGCGGCGCCCTCTGCCGGCAGAC
TCGGGAACGGCCACACTCGTCACCTCGCCCCCGGCCCGGCTCCTGGACCCCCCTGGACCTCTCTGCCAGC
TCTCCTCTCTCCTGGAAATGCGCCTGCAGGACTCGGAACGTGCAGAGGTCGGTCCTGGCCCCAGCCGTGC
CACGGGCTATCTGAGGGAGAGTCGCGGCCTCTCTGAGGCTTGGCTTCCTTTCCTGTGAAATGGCTGTGGT
GAGGAGCAAACGCTCAGCAGTGAAGAAGGACCCTTTGTAACCGCGAAGGAGCCTGTGAGTGTGAGGTATG
GTTGTTATCAACGCTAAGGGCAGTCTTTTGCCTTCTGCGGGGGTGACGACCATCCTTCTCCCTGCCCCAG
CCCCACAGTGGAAGGAAAACATAAGTCACCTCCAGAGATTATCACATGCAGTGGATTGCTTTCAGCCTTT
TTTCTGAATATAAAATATACATATACGTATTTTAATACAATTGTGGCCCTTCTCTACGTGCTGTTCTGTG
CTGCCTTTTTTTCTCTCTCTCCAAGTGCTTAACTTATGTTTTAAAGCCCAGATTCGGCCAGAGGGGTGGC
TCACCAACACTTTGGGAGGCCAAGGCCGGAGGATCTCTTGAGCAGAAGTTCAAGACCAACCTGAGCAACA
TAGCAAGACCTCGTCTCTACAAAAAATTTTTAAAAATGATCTGAGTGTCTGTAGTCCCAGCTACTGGAGA
GGCTGAGATGGGAGGCTCGCTTGAATCCTGGAGTTCCAGGCTGCAGTGAGCCGTGATTGTGCCACTGGCC
ACTGCACTCCAGCCTGGGTGACAGAGTGAGATCCTGTCACAAGAAAGAAAGAAGAAGAAGAAAAGAAAGA
AAGAAAAAGAAAACAAAAAACCAAAAAGAAAAGAAACAACCAAAAAACTAGAGTCAGACAAACCTGGATT
TAGGTTCTTGCTCTGTGACTTTATATTTCCACCCTTGAGCAAACCACACAACCTCTACCTTCCTCAGCCT
CAGTTTCCTCCTTTGCAAAATGGTGGTAATGCCAGGAATACCCACTTCACAGGCAGCGGTGAGGCCCCGC
ACACAGCCCAGTGCATGCTGGTTAGGGCTCTCCTTTTGCTTCCTGAGACTGCACGAGCTCTGCCTTCTTC
ATGCACCACGTGGTAAGGAAGGAGCTGGAGGCCTCCGCCTCTCGGACCTTGCTCACTGCTGCTTTTTTTT
TTTTTTTTTTGACAGAGTCTCTCTGTCGCCCAGGCTGGAGTGCAATGGCACAACCCCGGCTCACTGCACC
CTCCGTCCCCCGGGTTCAAGTGATTCTCCTGCCTCAGTCTCCCGAGTAGCTGGGATTCCAGGCGGCCGCC
ACCACGCCCGGCTAATTTGTCATATTTTTAGTAGAGATGGGGTTTCGCCATGTTGGCCAGGCTGGTCTCG
AACTCCTGACCTCAGGTGATCCACCTGCCTCGGCCTCCCAAACTGCTGGGATTACAGGGGTGAGCCACCG
CCCCAGGCCTCTTTCGGCACTGCCCTCAACCACATGGGCCTCACTGTTCTTCCGGCGTACCCAGACTTTC
CCCATCTCAGCGCCTTCCCACCGGTCCTGTGTTTCCATGGCCTTTCCCCGGGTAGGGGCCTTTCTTTTCC
TAGCTTCGCTCAGTTCCCTGCTACATTGTCGCCTCCTCGCAGAAGCCGTCTCTGGCCGTTCCGTATCGCT
AGCGCCCTCTATCATTCGCTAGCAGCTCACCCGGCTCTGTTTTTCTTCATTGCACTTCTCGTTATGTGCC
GTTCTCTTATAAATGTTTTTGTCTTTATCCCTCCCATAGAAGACACCTCCATGAAAGGCCAGGGCTTTCC
GAGGTTCTTTTCCTCTGTGGTTCCTAGAAGAGGGCCTGGCACATAGCCGGCCTTGGCTAGCTTTTTGCGT
AAATGTGAAGGGATCCACCTTCCTCCCTTATAAGAGGTAGCAGTACCTCCTGTTACCAGCAGAGGGCACC
ACCGTACAGCACTTGGGGGCCGCAGGGGATCCTGCTGGGATGGGAGGTATTTAAAAAGCCCACAGGCCTC
TCCTCCCATAATGTGGGCGCTCCCACAGCTGGGACCGGAAGCAGGAGGGCCCCCTGCGCTAATCTGCCCT
GGCAGCAGCGAGCTGGCACGCCCTGGGTGCATCATCCGGTGTGCTCTGGTGCCCAAGGTCGGAATACCCT
CACTGGTGGCTTGGACACACCCCTGAGCAGCTGATGCTTCTGGACAGATCACACCCTGTCCCGGGCTCTC
AAAATGAGGGCAATGACATGAGCTGCACTGCTCGCTCACGCGTTTGCTGTGAGGTTGGGACAGGAGTGCA
TGGGAAATGTTTTATGAATGTAAACATGGGACAAATATAATTTATGCACGCCTTTATTGGTAGTGCAGCA
GATTGACAACAAACGAGGGGAGAAGATAAGGTATTTAAAGGAAATTGCAAGGCTAAATGAAGGTGGGAAT
TAAGACAGTGAAGGAGAACGAAGAAGTTAAAAATAACAACTACCTTTCACCAAGAGCTTATTAGGAGCCA
AGGCAGAGGAAAGTGATCTCCAAACATTGTCTAATTATAGCCAACACTTACATGGTGCTGACCTCATGCC
AGGAACTCTTTAAGTGCTTTATGCATAAAAACTCACTGGATCCTCACAACAACTCAGGGAGGTAGGTCAT
ATCGCTGTCCCGTTCTACACAAGGAAACTGAGGCCCGGACAGCTTAAGTGACTGGCCCAAGGCCACATAG
ATAGACAGGGCTGGGCAATCTGCCTCCTGGAATGTGCTCTTAAAGCCCCACAGTCACAGCCACACCAAAA
GGGAGATATTGTTATTATCATTCCATTTCCCAAACAAGCAACTGAGGCTTAGCAGATGTACAAAGCCCGC
CTAAAGCCACACTGGGAATATGTGTATTTGAGCCCAGCTTAACCACCCACCAAACTAGGCCTGCAGGAGC
TAGGAGGGGCTTCAGGAATGTGCACAGGTTCTCTCGGAGGGCAGCAGCAGACATACCCACTCTTCCTCTG
AGCGGGGAGGGGAAGGTGAGAGCACAGAGGCTGGGCAGGGCTGATGGGGTTGAGGAAGAGGCTGCTTGGT
GGCCTCAACTCCATGGACCGCAGATGGCCAGGCATCTTCTGGCCAGGAAGTGAGAGGTGTGTTGATAGGC
ACAGTGGCTGTCCACAGGGATTCCAGCCAGAGCTGTGGGCCCCCCACAAATCTCCATAGGGAGACTAGGG
ACTGAGGGGTGGGCCTTACAGTGGGAGGTGGGAGCAGCCCCATCCCTACCACAAGTGCCCCTTAGCCCAG
CCCTGACTTCCAGCCCGTTACATAAGCTGCTGCATACCCAGGCCTGGAAATAGCTGAGGCTGTACCGGCT
GGGCTGGGAGACGTGATGAATGGTCCCCAGGAGCATCAGCTGACCGCCCTGTGCTCCTGAAGTGACCTGA
GGGCCTGCCAGCAGCTCGGGTTACCCAGCTCTCCCTGGGAGGCAGCCCTGCCTGGTCTGCTGAGGATGGC
TCTGATGTGATTCTGTGTCTGGACATTTATGGTTCGAGTGCCCGCAGGATGGTCCCTCTCCCAAGCCTGA
GCAGCTAAGGCAGCTCCCAGCCACGCTGTGCCCACTGGCAGCCTCTGGATGCTGGGAGCCAGGGCCTTGC
CCAGAGCAAGCACTTGGATCTCTGCAGACAGTCAGCACTGGGAGGGCTGAGAGCTCTGCTGCTCTGAGTC
CTGTGCTAAGGGCCCGCAAGGAAAGAGGAAGAGGAGCCTGAGAAACTCGGGGTGCACGTGGGGGCTGCAG
GTGTCATTCCAGGGCTGCACTCCACCCACATCCCCATGCCTCCTGTTTCACGGCCCACTCTGTTACTGAA
CTGGTGGCCCTGGGCACAGGGGGGCACCCACACACTCTGATGCCCACTGCAGAGGGCAGAGCCACACACC
CCTCCAGGGACTAGGCACATACACCCTTACTTCTGAGAGTAACGTGGCTAGGCTCAGGAGTGTAGGAGGG
CGTTCTCCCAGCATATGGTGATCATATGTTAAGAATTATTATTATGTTGGTCAGGTGCGGTGGCTCACAC
CTGTAATTCTAGCACTTTTAGAGGCTGAGGTGGGAGGATTGCTTGAGTCCAGGAGTTTGAGACCAGCCTG
GGCAACATGATGAAATCCTGTCTCTACAAAAAATACAAAAATTAGCCGGGCGTGGTGGCATGTGCCTTTA
GTCCCAGCTACTCGGGAGTTTGAGATGGGAGGATCGCTTGAGCCCAGGAGGTTGAGGCTGCAGTGAGCCA
AGATTGTGCCACTACACTCCAGTCTGGGTGACAAAGTGAGACCCTGTCTCCAAAAAAAAAAAAAAAAAAA
AAAAAAAAGAAGAGAGAAGTATGAATGATTATGGCCATGCCAATTCTGTGCCTTTAAGATACCTGCTTAA
GGGGCCGGGAGCAGTGGCTCACGCCTGTAATCCCAGCACTTTGGGAGGCTGATGCAGGCAGATCATGAGG
TCAAGTGATCGAGACCATCCTGGCCAACATAGTGAAACCCCGTCTTTACTAAAAATACAAAAATTAGCTG
GGCGTGGTGGCTCATGCCTGTAGTCCCAGCAACTTGGGAGGCTGAGGCAGGAGACTTGCTTGAACCCAGG
AGGCGGAGGTTGCAGTGAGCTGAGATTGAGCCACTGCACTCCAGCCTCGTGACAGAGCAAGACTCCGTCT
CAAACAAAACAAAAAAACAAAAAAGATACCTGCTTAAAAGACAAAAAGACCAGGCCAGGCGCGGTAGCTC
ATGCCTGTAATCCCAGCACTTTGGGAGGCCGAGGTGGGTGGATTACGAGGTCAGGAGTTCAAGACCAGCC
TGGCCAAGATGGTGAAACCCCGTCTCTACTAAAAATACAAAAATTAGCTGGGCGTGGTGGTGGGCGCTTG
TAATCCCAGCTACTTGGGAGGCTGAAGCAGAGAATTACTTGAACCCGGGAAGCGGAGGTTGCAGTGAGCC
AAGATGGTGCCACTGCACTCCAGCCTGGGTGACAAAGCGAGACTCCATTTTAAAAAACAAAAACAAAAAC
AAAAACAAAAAGACCAAAATTCAACTTCTCCCTAAAAATATTAATGCTTTAAGTGAAATTATCTCTCTAG
TATTCCCCAAATCCTTTAGTGGAATAGGCATAATAGGCACTCCCAGGGAGCTAGGACCTGTTTACTCTGA
TTCCAGGACCTTCTGACAGGTGGGAAGCCCCCAGCTGGAGAAGTGTGGGACATTTTCTAAAAGAGCAGTG
GAAAGTGAGTGAGTTGCCCCATCTGCCTGACCACAGATTGCCTGTCCAATGACAGGAGGGACGCATACAT
GCACACACACTCCACCTCTCTCCTTGGTGCTCAGATGTGGGGGCAGAATTCCCCTCCAAACGCCTGCAGC
AGCATTCCCTGAAGACCCTGCCTGTGCCCAGACCCTGCCCATGACTTTGCACCCTCCTTGTGTGCCTGTG
GGTCCTGCAGTTCTGAGCCCATGGCCAAGGCACAGGGGAAACAGGCCCCTAATGTACTCCAGAGACGGCC
GGTGTGGTGGCCCACACCTGTAATCCAACATTTTGGGAGGCTGAGGTGGGTGGATTGCTTGAGCTCAGGA
GTTTGAGACCAGCCTGGGCAACAGAGTGAAACCCCGTCTCTACAAAACATACAAAAATTAGCCAGGCATG
GTGGCATACACCTGTAGTCCCAGCTACTTGGGAGGCAAAAGTGGGAGGATCACTTGAGCCAGGGAGGCTG
AGGCTGCAATGAGCTATGGTTGCATCACTGCATTCAAGCCTGGGCAACAGAATGAGACCCTGTCTCAAAA
AACAAAAACCACACACACACAAGACAGAACACCAGAGAGCTAAGGAATACCTACCCCTCTGCCATATAAA
CCTGTGTGGGGAAGGGCTTGGCTTCCTTCAGTTCCCTCCCGCCTGCCTCATGTCTCTCCAGGCACTCTCC
CTTGAGGGTTGTCACCGGTAACCAGAGCTGGCTCCCCTGGTGAGAGCAGGAGCAGCGTTACTTAGCAACA
GGCACAGCACACAAAATCCAGCTAATCTGGGCTGCCCCAGTGGCCAGCCCTCCTACAGCCCTCAGGGCGG
TGGCTGCTGGCCAGCAGCACACCCCTAGAAGTGGACACTTATAAACTTCTGCATCTTTCTACCCCAGCAA
ACCCTAAGTTCCAGCTTGGGCAAGAAGGACTCTTGCACACAAAGGCACAAAGACATGGGCAGGGTGTGGG
CACAGGCAAGGTCTTCAGGGAATGCTGCTGCAGGGGCATTTTCTCCGCCCTCCCTGTCCCTCTCCCCTTC
CTTAGGCCCTGTCTCCCTTCACAGTATCTACAGAACTCTCTGAGGCGGGGGTCCCAGCTCTGCAGCAAGC
CATGAAATGAAGGGCAAATCCCTTCACCTCTGGGTAGCTCAACTTCTCTGGGAAGCAATAACATAGCAGC
TCAGTGTAAGGACTTTGAGGCCACACTGGTCTGGATGCAAATCCATGCTCTGCTACTTCTCAGCTGTGTG
ACACTGGACATGTTTACTTAACCTTTTATTTATTTTTTATTTTGTATTATTTTTTGAGACGGAGTCTTGC
TCTGCTGCCCAGGTTGGAGTGTAGTGGTGCAATCTTCGCTAACTGCAACCTCCACCTCTCAGGTTCAAGT
GGTTCTCCTGCCTCAGCCTCCTGAGTAGCTGGGATTACAGGCATAGGCCACCATGCTGGGCTAATTTTTG
TATTTTTAGTAGAGACGGGGTTTCATTATGTTGGCCAGTCTGGTCTCGAACTCCTGACCTCATGTGATCC
GCCTGCCTTGGCCTCCCAAAGTGCTGGGATTACAGGCGCGAGCCACCACACCTGGCCTACTTAACCTTTT
AGAGCCTCAGTTTCCTCAATACTAAAATAGTGCCCACCTTAGATGGCTGCTGAAAGAAGAGACAAAGGAA
AAAAGTCATCGGCACAGAGTCTGGCACAAGGGCCTGTGGATTGGGCTGAGTGCTCCTCTCGCGCATCCCC
AGCTCTGTCCCCCTTAGTCACTGGCCTCCAGCCACTTGCCTTACTCTGCTGCTCTAACAAGCTGGCACTC
TTTCATTACTGTTTCCTCTCCCTGCAACTTATCCCTTTCCCCTAACTCTCCACCGACAAACTCCCCTCAG
TTTATTTATTTTGAGATAGGGTCTCACTCTGTCGCCCAGGCTGGAGTGCAGTGGTGCTGTCACAGCTCGC
TACAGCCTCAGACTCCTGGGCTCAAGTGATTCTCTCACCTCCGCCCTGAAGTAGCTGGGACCATAGTCCC
GTAACACCAAGCAGGCTATTTTTTAATTATTATTATTACTTTTAGTAGAAACAAAGTCTCCCTTACGTTG
CCCAGGCTGGTCTCAAACTCCTGAGCTCAAATGATCCTCCCATCTCGGCCTCCCAAAGTGCTGAGATTAC
AGGCATGAGCCTCCAGTGGTGCCCAGCCTTCCCTCAGTTTAAAAGCTACTTCCCGCCAGGCACAGTGGCT
CACGCCTGTAATCTCAACACTTTGGGAGGCCGAGGCGGGCAGATCACATGAGGTCAGGAGTGTGAGACCA
GCCTGGCCCACATGGTGAAACCCCATCTCTACTAAAAATACAAAAATTAGCCAGGTTTGGTGGCATGCGC
CTGTAATCCCAGCTACTCATGAGGCTGAGGTAGGAGAATGGCTTGAACCTGGGAGGCAGAGGTTGCAGTG
AGCCGAGATCATGCTACTGCACTCCAGCCTGGGTGACAGAGTGAGACTCCGTCTCAAAACAAACAAACAA
ACAAATAAAAAAGCTACTTCCTTGGAGAAGCTTTCTCTTATCCATCCCTTGCGAAAATTACAGCTGCCTG
TTAAATATCTCACTGACATCCTCCATTTCATTTTCTCTTTATGGTTGTGATGGTTAATTTTTTTTTTTTT
TTTTTTGAGACGGAGTCTCGCTCTGTTGCCCAGGCTGGAGCGCAGTGACGCAATCTCAGCTCACTGCAAC
CTTCGGCTCACTGCAATTTCCGTCTCCTGGGTTCAAGCAATTCTCCTACCTCAGCCTCCGGAGTAGCTGG
GATTACAGGTGCCCGCCACCACGCCCAGCTAATTTTGTATTTTTAGTAGAGACGGGGTTTCACCATGTTG
GTCAGGCAGGTCTGGAACTCCCGGCCTCAAGTGATCCGCCCGCCTCGGCCTCCCAAAGTGCTGGGATTAC
AGGCTTGAGCCACCGTGATGGTTAATTTTAAGTGTCAACTTGACTGGGCTAAGGGATGCTCAAATAGCTG
GTAAAATGTTATTTCTAGGTGTATTTGTGAGGCTGTTTCTGGAAGAGATTAGTATTTGAATCAGTAGACT
GAGTAAAGAAGACCGCCCTCACCGCTATGGGCAGGCATAATTTAATCTGTTAAGGACCTGAATAGAATAA
AAAGGCAGAGGAAGGGCAAATTTGCTCTTCTTTGAGGTGGGACACCATATTTTCCCTGCTTCAGACATCC
ATGCTCTGATTCTTGCACCTTCAGACGCGGACTGGAACTACTCCAGCTTGCGGAGGGCAGATCCTGGGTA
CTTCTCAGCGTCCATAATCATGGGAGCTAATGCCTCATAATAAATCTTTTTCTATATCTCTGTATATCCA
TCCTATTGGTTCTGTTTCTCTGGAGAACGCTGGCTAACACCATGGCACTTACTGCTTATGTGCAAGATTG
TTGAACATTCATCTCTCCTACTTGGCTGTCAGCCCGGGGACAGGAGCTGTGTCTGTTTTGTTCTGTGCTG
TGCTGTGTTTCCAGTGAGAGGGCTGGCCTAGGTGTTTCTCCGAAGTATGTTCCAGCTTTCGGTACCTCTT
TATTTCTTATTTCTTCCTTCCCCTGCTCTCCAAGTTGACCTAAATAGTTTTTGCACCTTTTCTTAGGTTC
ACTGAATCTCCAAGCAGCGAGGTGTATCTGGAAAGACAGCTCAAGAATTAAGGGTGCTCACCCTTAATTA
CACTCCATTTTCCTCAGGACAGGCTGGAACAGATTTGGGATGGCGAGCCCCCAAACCAGCACTCTACAGA
TTTCGGGACCCCTCTTAACTCATTTCCCCCATCCTCCCAATGACGTCTATTCGCCTGCAGCAGACCCGAA
GGAAGCTTCGGCTCAATGGTTCATTTGCATGCGCCCGTGCTATTGGTCGGAGGGGCGGCACCGTAGCCAA
TGGTGTGCAGGGGTGTTGACCGAGCCGCAGCTCTGCGCCGCCGGGTGAGGTAATGGAATCCTGGCTGGAC
CTGCTGCGGCTCCCTCCGGACTGGCTAGTGACGTAGCGGCTTCCCCGGCCCCACCCCGCCCAGCCCGGCC
GCCCCCGGGAGCGCAGCTGGGGCTCGGCCCTGTTCCTTCCGGCCCGGCGTTCCGGTCGCGGGCCCCTCAG
GGAGGACCCGCCGGGCCAGATCCCATCTCTGGCGACGCGGAGAAAAATCTGGAAGCCGTCGTCGGAGGAT
GACCAAATCCTCGCTCCTCTGGGAGTGCGATTACGGGCCTCTCACTTCGTAAATCACGCAGGCCTGAAAC
GCTCACATTTGTTTACCACGAGCATGTTTGATTTTGTAATTTAACAAATAAAATTGCAGAGGATTTTTAA
GAGCCCTGGCCTGGATTTAGGGGAAATCTCGCATTAATTCCAGCTCTGCCATTTCCGGTCTGGAGACCTT
GGCCACTTTCTCTTGGGGCCCTGACGCCTTCCATGGCAACTAAGTAGAGCAAACGTGGAAGTGCTTCGAA
AACACCAAGTGGGGTCTCCTCATCCTTATGCAATCTGGAGAGTGTAACCCCATCTGGGCGGTGTCTCTGG
ACAGCCCCTGAGCCCTGAGACAGCCCCAGGAAGACCCGTCTTTGTGTCTCTATTTTTTCGGCCCTTAAAT
TGCTCTTAAGCCTGGGGAGAGGTCGGGGGACCCCGGCCAGGTAGTTCTTGATCCTCAAGAAGAGGAAGGA
AACCATGAAGGGAATCCCTTCCACAGCCCCTCTCCTTATTCAGGGCCAGGGCCTGAGCAGGGGAGCCCCT
TCTCTGCCCAAATGCCCTAGGGAACCCCCTTGACATCCTTTTACCCCCGCCCCCGAGGGCCAGGATGGAG
GTGGAGTGGAAGGGTGTCTCCTAGGTGGGGTGGAGTTTCAGATGCTGTGAGATGGAAGGGCACCTGGGGA
GGGCCACTGAGAGTCTGGCTTATGGTCCTGGGTCCTGTGGCTTACCTTGGCCCACTACTTCCCACCCTCA
GGATCTCAGCCCTGCCCTGCCGCATACCTAAGGTTTGGGGGCAGCCTCTCTCCTCCATGGAGTGCCTTTT
CTCTATACCCTGCCTGGTCCTGTCTCTTCTTTCACACTGGGCCCTACATTCTCTTCCTCCAGGGAGGCCT
CTTGAGACTAGTCCTCTAGATTCTACCCCAATGCCAGCTCAAGGCTTCTTGGAGGCCACTTCTTGGATGT
CCTGGCAATGTTTCCATACCTATTCTTGTGTCCTGCCTCCCCATCAGACTAGGAGCCAGCTTTCTGTGTA
GGAGCTTGTCCTCTAGGAACCTCCCACAATGTGGGGCGTCACTGGCCATGGTGGCCTGTCTCCCCTCTGC
TCCGACTCCCTGGGCCATGCACGTTGTTCACCTTTTCTCTCCTCACCTTTCCTATCTGCAATAGGGGCTG
TGAAGCAGTGCCTGTAGAAAGGAGAGGAATTAGATATGAAGGAACAAGAGGCTGGGTAGAAAGAGCACTG
GCCTGGGAGTCAGAAGACCAGGGCTATGGAGACAGAAGACCAGGGCTATGGTTCTGGCTGGGCAACTCAC
TAGCTGAGTGACCTGGATCAGGGAGACAACAACAGGGCAGACGTTTACTGGACATTTGTAATGACTAAGA
AGCGACATTGCTCCTATCTGTGCAGCCCCCACGGCCCCCAGTATTCCCAATAAACATACTGGCTGGGCAA
AGCTAGGGTAAGCACCTTGGATGTACAGCTTCAAGACAAGGTCCCAAGTCCTCCAAGTCCTCCTGTATGG
AAATGGCAGATCTGCCATGATGTGCCAGACTCCGCTTCATGCTTTATGTCTGCTCTCAAAGAGGCACTCT
CATGGCAACCCTGTGAGATAGGTTTCTTATCATCTTTGTGTTTGAGGTGAGGAAACTGAAGCCCAGAGAG
GGCAAGTAACTTCCCTGGAGTCATGCAGCTGGTAAGAGGTAGAGCCTCTTGTGGGTTAGGGAAATGAGAC
TCCTAGGGCAGAGCAGGGAGGAGAACTAACTCTGAGTGCCTGCTGTGTACAAGGTACCTCCACATCTGTT
CAGTCCTCCGACAACCAGTGTCCATCCCAGACCTCTAGAGACTGCTTCTTCCACCATCTCACGCCGCCTC
CACATGCCACCCAAAGCGTGCGTGTTCTGGCATTCCACATTTTAGAATATGTTTTATTTGATTATAAATA
GGAGAATATATAATAAAGACAAGTTATGCTAAAAGTATTTATGGAAAGGAGAGACCTCTGTGGGCTGGAA
GCCTTCATGGAAAAACTGGTAACTGAGCCCTTTTTTTCCCTTCCTGTCTAAATAAATGTATTTCTCCCAC
ACAGGCAAATATTTTAAAATGTATTTCAAAAGTTAGACTTCTGGAAAAAGAAAGGAAAAGAGGGGGTGGG
GTAGCTGCTCCTCTAAGAAGCCCATGTGAAAGAAGCTGCTGGGGTAGGAAACAGGAGGGCACAGGGTGAG
TGAGGAAAAGTCAGTCCCTTTCCCCCACCCCAACGCCTTGTCATTTTTGAAGCCTCAGTTTCTTTATCTG
CAAATTGACCTAATACTTGAACCTGGGAGAGAAATGTACAGGTTCTTGGGATGTGATTTTGAGAGACAAA
CTCTGCAGAGGTTCTTGGAATCATTATGAAGAATGAGAGAAAGGGAATTCTGAGTTGCGCCAAGAGTTTA
GGATTTGTCCAGCAAAGGATTTCTGGGGGTGGGCAGTGGGGTGGAGAGAGGAATGCAGCCCCTGGAATGA
GGGCTGAGTGGAGGTGTGGGGCCTCTTTTGGAGAATTTTGAGCACAGAAGAGATTCTTTGCTGCGTGGGG
CCTGGGCTGGGTGCTGGAGATTGTATAGCATGCTCATAATTTAAGGTGGCTCCAAAAAGGATCAGGGGCT
AGTGGCTGGCACAGCAGAGCACATGACTGGATCCTCCCTAATGGGTGTTCCCCAATGACCTTGCCTGTAC
GGGGGTATAGGAGCAGAGATGGTAACCCCCAACCCCACCCAGCCAGCCCCAATCCCATTGCTCCAACTTT
GGCCGGGCCTTCCTGACCAGCCTCTCCTGAGTGGGCAGCTGCTGGGGGAGAAGGATTTGCTCAGGACAGG
ATGGTTCTTCCATTTCCAAAGAGGGCTCCAGTCACATGGGAGAGAGAAACCTTCCAAGTGCCTGCACCCC
CAGGAAAATTCTTACCATGGAAGCCTGGGCCTGAAGACTCTGGTCAGAGGAGGGTGGGCTTCCCAGGGCT
TCCTGGTCACGGTGGTGGGCCTCTGAGTGCATGAAGCACTCCTTTGGATTGGGGAGAGCAGGGAGAACTA
ACTCAGTATCTACTGTGTACAAGGCACCTCCACAGCTGTTATCATCCTGACAGCCAGATATGGTGGATAA
TCTGATCATATCATGTTTTAGAGACGAGGAAGCTGAGGCTCAGGAGGTCAAGTGACTGTCCTAAGTCACA
CAGTGGTATGGGAGCAGCCAGATCAGTGAATCCATCACCTGGGCTTTGCCGGGGCCTGCACCCCCGCCTC
GGGAAGTGCCCTCTTCAGGAGAAAGTCCTATTTCTCTGTAAGCTGCCTTCTCGTGTGGAAGCTGGGTGCC
CTGCTACCTGGGTTCCTTGCCCTTGGGGTGGCATGAATGCCAAGCGATGCAGGCAGGGCGCCCGCAGACT
CAAAGGCCAGGCCCTGTCCTCGCCAGCAGCCGAATCGCTGTGACTTTGGACCGCCACCGAGTCTCTCTGG
GCCTCGCTCTTTCCACCTCTTCCTGGGGAAGCCCCGGCTAAGTTTCTTTATAACCTAGCCCCAGGTGCCC
AGCCTCGGAAGGTGTGAGCGAGACCTCGGCACCCTCACCTGAGAACCAGCTCCCCGGCTCTGGCCGCGGG
TTATGTAACCGGGTGTGCGGCGTGGGGGGAGCCGCGGGGCGGGGCGCTCGCGGCTATTCCGGGCGCCGCT
ATTTTTAGCCCCATTGATGAGGCTGCGTCGGCCGCCGCCGCCGGGATTCCGGAGACGTCAATGGGCACGC
GTCACGGTCCCCGCCCCGCCCGCCGGGGGAGGCGCGCCGGGGCTGGGACAGCGCGCGGGCGGCCGCGGGC
GCGGGGGGGGGGGGCGCCGCGCGGCCGGAACTGCGGGCGGGGGGCTTCCCGGGGGGGGGGGGCGCTGAGC
GGCTGCGGCGCGGCTGCAGGAGCGCGAGGAGGAGGTGGCGGCGGCGGCGGAACTGGCGGGGGTCCCCTCG
CGGCGCCCGGGACCCCCGGCCTCCAGGCATGGGCGCCCCTGCGGTGCAGAGCAGCTCAGGGCCGGCTGGT
GCGCGACCCCGGAAAGCGGGGGTGGAGAGGCGAGCGGAGCCCGCGGGGCCAGGTGAGGGGCTGCCGGGGT
GGCGGGAGGTGGGCCCCGGAGAGGCCGGGCAATCTCGGGTCTTCGGTGGATGCCTTTGGGCGGGCTCGGG
GCCACGCCGGAGTCCCGCTTGGAAACTGGGGAGTCGGGGGGGGGACTGGATTCCCAGCGGAACCGCTGGC
TCCGAGTCAGCCCCCAAGGGCGACGGCCAAGGCTTTCTCTCCCCAGTCCGCCAAGGGTGGGGTTTGGCCC
ACGAGGGGCGCTGGAGGTGGCCAGGCCAGAGAAATTCCTTCCATTTCTCCGCCGGCTGCGGGAACCGGTT
CCAGGAGCGCGGGAGCTCGTCTGCCAGGAGACCCAGCCGGGAGGGACCGATGCTGCGTGATGCAACCCCT
TCGAGGGACTGAGTCAAGAAACCAAGGCTGGGGGTCTCGTGGAGTCAGGCGGAGCTGGGGATTAGGACTC
GGGGCTTGTGTCTCCCAGGCGTGTTTTTTTGTTGTTGTTTTGTTTTTTTGCCGCAACATCTTCCTTATAC
TGGGTGGGGTGGGGGCGGGGGTGGTCTGTATTTCCAGACCTCCTCCATGGTGGTCTGGCTGTGTGGAGGG
CAGGGCACAGTTCCTTGACTTGGCTCATTCCCCCCCAACGCCCCCCCTTTCTTTTTTTTTTGAGATGGAG
TCTCGCTCTGTTGCCCAGGCTGGAGTGCAGTAGCGTGATCTCGGCTCACTGCAATCTCCGTCTCCCGGGT
TCAAGCGAATCTCCTGCCTCAGCCTCCAGAGTAGCTGGGACTACAGGCACCCACCACCACGCCCGGCTAA
TTTTTGTATTTTTAGTAGAGACAGGGTTTCACCATATTGGCCAGGCTGGTCTGGAACTCCTGACCTTGTG
ATCCGCCTGCCTCGGCCTCCTAAAGTGCTGGGATTACAGGCGTGAGCCACCGCGCCTGGCAGGCTCATTC
CCTTTTTATGCCTTCGTTTTTGATAGATGACCTGGGGATGGGAGAAATGCTGCAGAGGTGGTTTATTTAC
AGTGAATTGTTAATTTGGTTGTGGGGCTAGTATTAGGGGCACATTGGGAAAACACAGGCTGGGCAGAGGT
TGGTAGGTTCACAACAGTTATGCCCACTGGGTGCTGATGGATTTGGGGGCTGTCGTCGGCCTGGCTTGGG
GCCCTAGTGGTGGGGCTACAAGGCTTGCTCCTAGATCTACTCCAAGTGAACATTTAAGATCAGTGACTTG
GATGGAGACTTAAAAAGGAAGTCAGCTGACTCCGTGTAGTCATCCAGAGCTGTGAGGAATGACAAGTGCA
CAGTATAAAATCAAGATTCTAATTGAAGGTAACTGGGCAGAAGGATGGGCCGAGATTAACGGTGTGAATG
TGACTGACGATTCCTGTGCTGAGGCCTCTGCTGGACTTAAGAAAGTCAGCTGAGGAGTTCAGGCCCTGTG
CCTGGCTCAAAGGCCCAGGGACCCAGGCCCTTAGCTTCTGCCCTGCTCTCTGGATGGAGGCTGTGGGACT
GCTCTGTGCAGTGTGAAGCCTCCATCCGCTACACAGGTCCAAGTGGGTGGCAGGGGGAGCGAGAATGCCT
GATTCTGGACTCCTTGGTTAGGCTCCCTGGGTTCTAATCCCAGTTTTGCCAATTCCTAGCTGTGTGACCT
TGGGCAAGTTGCTTAACATTTCTTTCTGGGCCTTAGTTTCCTCATTGGTAAAATGTGCATAATAATAATC
CCTTCCTCATAACATTGTGAGGCTGAAATGAGAAAATGCATGTAAAGTGCTTAATGTATCTGGCACATGT
TGTTTAACAAATATTAGCTGCTGTTGTTATTGCTAATAATAGTAATATGCCCACTTCTGGGTAGGGATAT
TGGCCAATGCAGAGCTTGTACTCAGGAGGCCACCAGGACCTGTCACGCAGAGGGCCTGTCAGCGGTGTCC
TGTTGCAAGGGGCAGTTTATTAAGAAAAGACTAAAATACTTGCTAGGAAGGACTAGAATGAGGCTTCCTG
TGGGCCAGTGCAGGGGACAGAGCTGGGATCCAGGGCTGGGGACTTTTAGAGTCAGGGATGTGATTCAACA
TAAGGAAGAACCTCTGCTCTGGCTCTTGGATCTGCCGCCAAATGGAAGGGACAGCTCCTGAGCTGGAATG
CTCTCCCCACCTAGCTGACCTTCCTCCCAGCACAGCTCCAATGCAGGCATGTGCATCCACCACCCTTCCA
CGCACACTTACTGAGCACCTGCCAGTGCTGAGCAGGGTTTAGAGACTGCAGACTCTGCAGTGATTGAGAT
GGACAGCGTCCTTAGATTCTGGTTGGGTAGACAGGTGATAAACCAGTGAACAGATGTGAACATTAGACCT
ATGATGAATAAAATCAGGCTAGTGTGGTGGGGTGGGGATGCAAGCTGTTCAGGGAAGTGACATGGGAATG
GAAACCTGGTTGATGAGAAAGAGCCAGTTCTGGGCAGAGGGAATGGCAGATGCAGAGGCCCTGAGTGGGG
AGAGAAGGCCCACGTGATGGGACCATAGTGAGTGGCTGGGAAATGAGGTTGGTGGGGAGCAGGAGCTGGG
GACTGAAGTCTGGCCTGATGGTCCACCCCATTTTGGGGGCCACTTTCTCCTCCTCCAAGCCCACTCAGCC
TTTGGTTTGCTCTCTGCACAGTGCAGACTGTACTTGACCTTGGTTCATCCCTTATCAGCTCCTGGGGCTC
TGGGATCTTGGTGTGCTCATTCTTGCCTCTCCTGTTGTTCGGGGCACACTGCCTGGCTCCCGGGGGTCCT
CAGTAAATGCTTGTGATGTGACTATTGAGGGGGGCATTTTGATCTGGAACAAGTGGGGAAACAGATACCT
GTTTGAAGATGCTATGAATCATTTGTGGGCACCACCTGGTGCCAAACTCTGGCCCCCGTTCTGAGAACCA
GATGGGGAAGACCTGGGCACTCAGGTTGGGCCTGACTATGGTAGGTGGTATTTGGCCCTCCCTACCCTTC
CTCCTCAGCAGTGGGGTGGTAGGGAGAAAAGACAAAGCTGGGGGAAACTACAGACCAGTTTCGGGGGGAT
GGGAGGATGACACTGTCATCCTGGAAAGCAGGTGTAGTCCATGGGCCGGGGCAGGTTGGAAACCTTGTCT
TGCTTTCTGTTTGGGGTTAAAGGTGCTCCAGATGTTCCAGTCTGCCCTCCCTTCCCTCCTTGTCTCCTGT
GGCCTTTTCCTGCTTCCCATCATACCCACAGGATCGGGAGGGAGGGGTGCTGACAATGTTGTAAGGGGAG
AGCCTGGGTACTGTGGGCCAGAGGATCTTCCCTTGGCCTGTAGACCTTGGGCAAAGGTCAGGTGGGTGAG
GAAGCCCATTGCCCACCCTCTGGGGTCCTCCTCCTCTTTGCCCCTCCTAGCTCCCATCATCTCCTTCTCT
TTTCCCAGACCTCATCTCAGTCTGAAGGGCAGTCCAATAGCCAGTGAAGGCCTGGTCCTGCCCTCACTCT
GGTGCGTAGTCGAGAGGAGAGAGCACTGGAGGGGATGGAAGAAGAGAAGGGGAGATGTGGGTCAAGGTGC
CGTGGCTGAATGGGGTTGGGGGAGAGCTGAAGAGGCTGGGGTGTGGGGTGAGGCACACAGTTGGCTTCCA
CCCTGGAAGGAGAGGCCAGGTGCCCAGGGTACCTTGGACTAGGAACCAGGGGTTGGGGCTAGCTTTTATG
TGTGTGTCCTCATTGAACTTCTCCTAATATTGATATGTAGTAGATATGTGTATCCAGCACTGTCCTTAAG
TCTTTTACACATTAACTTTTTTTTAAGCCTGCTGATAAGCCTGTGATTGTTAATTTTACAGATGTCATCA
TTTTTACAGATGAGGAGCATGAGGTACAAAGAGATTATGTAATTTGCCCAAGGTGATAAGCAGTGGAGCT
AGGATTCGAACCCAGACGGTCTGGGATCCCACATCCACTCTACTCTAAGTTGAGTGGAGCCCCTGTCTCT
TGGGGGCCTCCGAGGCTTTAGGGGCCAAACTGGAGCCCCTTAAGGAGAGTGGGGTATTCCGCTCTGTCCT
CACAATAGCTCCCTTTCTGTGCCCGCAGCATGCCCTTTTGTGCATGTCAAAGGCCGAGAGCCAGGAAGGG
TATGAGGTGGAGTTGCTAGATGCTGATGTGTCTATATGGCTCTGAAATGCCCCAGATGCTATCAGCAGCT
CCTGGCCACACCCTACACCCCTGGAAGCAGATTGCCTGGGGCCTCGGCCCCCAGGCTCCAATGCCTAACC
CGGGGAGGTCCTTGGGCGCTGGCTTGTCCCCCTCTTGTGGCCTAGGTCCTGCCACTGCTCCCATTCCTGC
GTGTCCTGGGCATGCTGTCTCCAGGGAATGTGCCTGCTGGCTGGTTTCTCTTCACTCACATCGACTCTCC
CTCTGTAGGCCTCCACCATGGACAGAGGCCAGGCCCTGCCCCTCCCAGGCAGCCTGGCTCCTTCTGCTGG
GCCCTGAAGGCAGACGGGATAATGTGGTTGGCCAAGGCCTGTTGGTCCATCCAGAGTGGTAAGTGCTCTG
CTATTGTCCTTTGTACACCCCTCCAGCAGGTGGGGTTGGGGGGCAGAGGTGGGGGTGGAGCTGCCCAAAG
CGGATGTGGTTAGGGGGATGGAGGGGAAGCCCTGGGGAGGTGGCAGCCGAGGTGCTGTTCAGTGTGCGGG
ATCTGATTTCTTTAGAGGTTGGGGTAGTGTGAGTGTGGGGACTGGGGAAGCCTTATTTTCTTGGCAGGAT
AGGCTGGAGGGCTCTCTGGGCCTAGGTGACACGGGCTGGTGGGAGTAGGGGATGACAGCTGCTCCCAAGG
CAGCATGTGCCTGATTCCAGGAAGCAGATAGGGTAGAATAGGGTGTCTGGGGGCTGGGCTGGGAGCATCA
TGGGGCAGACGTGGCAGCGGGGAACTGGCCTGTCTCCCAGGTGTAGACACTTCTCCAGGTGAGTGCATGA
AGGGAAGGCTGGGACCTCCCCTTCCTCGTGGAAAGCATGGTGCCTGCCCCTCCTCACTGTGGCAGGCCAG
AGGGCCTATGGAGTCTGTGGTGTCGGGGGTGGCCTCTGTGAATGGGAAGGCAGGGACAGCCCTTCCACTA
CTGGTGTTCTAGCACATCTCCTTCACTTACAGAAGAGGAGACTTAGGTACAGAGGGGAGAAATGACTTTT
CCAGGGGTCCTGCAGTGAGGATGTGTAGAGCCAAGCCCAGAACCATGCCTCCTGGGTGGCTGGCAAGCAC
AGGCACCTGGTCTGACTGTGGAAGACATACCTTCCTTGACTGAGGGTGCTTCCGGAGAAGGCCCACACTG
ATCTGGGGCTGAGAGGTGGAGGACGGGAATGGGATTTTCAGCAGGCCTTTCCTGGGGACATGAGATGGGG
CCAAGGGCACTGTAGTCCTGGTTCTGTGCCTAAAGGAGCTGGAGCTCTGCCTTGCACCCCAGACAGGGGT
CATGAGATTCCCTTCCTCCTTCCCTGGAAGAGGGCCATCTTTGGGCAGCAGCCCTCCCAGGCCTTGAGAA
ATCCCGGATGAAGCACGCACTGGCTCAGTATCTCCCTCAGCTTCCCAAACACTGTTGCCTCATGGGGGGT
CAGCGTTCAGGGTGCAGAGAGCCCCAGAGAGGTGGCTGGGGTGGGGAGGATGGGGTGTCTGGTCCCAGCC
TGGAGGGAAGGGGGAAGCCCTGGGAAGCAGGGCCTCCTGTTCTGGGGGCAGGGCTGGGTGTGTGCCTGGC
CTCCTGATGGCAGCAGGTGCTGTACCACCAACCTCGGGCTGAAGACAGGGAGAGGAATTACTGTAAGGGG
GAGAGGCAGGTGTTCTCTGGTCAGCTGGAGATCTGGACTTGGGAAGCACGTGAGTGAGGATGACAGCAGT
GGCCAGGTGTCCCTCCCTCAACTCGGCCCCTACATCCGCCCCCAGTCCAGACCCAGCTTCCTGAGCACTC
AGCCCTCCCTCTGGGTACACAGCAGCCCTGGTGGGCAGAATTCCTTGTGTTTACCTCGTTGTCTGGGTGA
TGGTGGTGATTGGGTGGAACTCCCATGCCAGATATACTGCCAAGGCCACCCTGAGGGGCTGCCAACCGGG
GCCCCATGAGTGCTAGAGCTGCTGGTCTTGTGACGTGGGCACCTGTCTGCCAGGCCTGGGCACGGGCCCA
GGGTCACGCTCATGCTGGGGCTCCCGTCAGCTGACTATTTTGGGCTCTTGCTGACCTGGGCCAAAGTCAG
GTCCAGTCAAGAGGGCCCAAAGTGGCTCCCCCCATCCCCAGAGGCCGGCTCTGGTTTCTGCTATATAAAC
AGAGGAGGACACGCCGGCTTGGAGGCAGCACCACATCCAGAGCTCGCTCAGCTGCTGCCCAGCCTCGGCT
GTGAGGATAGGCTGGCTGGGCAGCACGTCTCTCCCCACAGGGCTCCCTGAGACCACCAGGAAGAGCCCCC
AACCAATCTTGGGATTCTCCCTTCGTGCGGTTGTCTGGGACCTTTTTCCAGGGTCAAAGCAGATCGTGAG
GAGGAAGCTGGTGAGTCTTGGGACAGGGACATTGAGGAATGGGAGCTGGGCTGTTTGGATTTTTTCCCAT
TGTGACTGTCATGCCAAACCCTTTGGCCTTGGGCTGCGGGGAGGAAGCAAGGGTGGCCGACAACTGGAGG
CATGAGTGTGCAGCTTGACCACAGCTGGATGACGGTAGTTGGACTTGTGAACGCTGTGACTGAGAACTAG
GAGAGGTGTACAGAGACAGCTAATGCAGTCTAGCTCCTTTCTCAGAGCTTCCCCACAGGGTGTCCGTCCT
CCTCTCCTCTCCACCCCGCCTGGATGAGGCACTGGTCACTGCGGCGAGTTTGGTGTCCATTGGGGTATCT
CAGGATTGGCTTCTGAGTGGGGCCCCTGAGCCCCCACTAGTTGGGACTGAGTGGCTGGGCCAGGGGCTTG
GCGTTCTGCTGAGCCCTCAGTCCCTGGCCTGCCCCGCTTGCCGGGCCCTTGCCCGGCCTCCCAGCTGCTT
TGGGTGCCTGAGAAGCAGAGCTGGAGAGAGGTCTGGCAAGTCCGCCAACCAGCTTGCCACAAACACGCTC
CGGGACCTGGGCCAGGGACTCTGGCCCGGCATGGAACAGGCAGCAGCTCCTTACGTGGGAGGCTCCTGTG
CTCTCCTTCCCAGTTCTCAAAAAAAAATTTTCCCCTTTCTGAACGTGAGTCTCTAAACGAATCCAGAGCC
TGTGACTAAAAATACTTTAACAATAGGAAAGTGCATCAGTCTTTCCGGCTGGGACTCGTTATCAGAGAGT
TTTTATTTTGGAATTTGAAATTTTGCCCACGTAGGCTGGGTGGAGACCTTGGGCAGAGGCAAGGGAATTT
GGTGGGTGCTGATGTTCTGCAAGGTGGTGGGAGACCCTCCAAGGCGAGAGGAGAAGAGAGCTGGCCAGGA
GCTGAAGAGACTGAGAGGGAAACTGAGGGAGGTTCAAGGCGTGGGGCAACCTTTCCCAAAGGAACTCCGT
GATCTTGTCTGTGTTTTCCAGCCCCGTCTGGTGGTTCTTCCTGAAGTCTGATCTTATTCTTTTATGCTGT
ATGTTAATTTCTTCTGGCCTGAGAGGGAGGGAGGGAGGGGTAGAGAAATCACTGGACTGAAGATAGCCTC
TGTGGCCATAGCCTGACCTCCTTAATTAACTAGCTCTCTCCAGAGTATTAATTGACCACTCGCTGCATAC
ACAGTCTAGAATTCTCCCAGTGCTGGGTAGAGCCACATCTCTGGGCACACTCTCTTTTCCTCTCACTCAG
CCCAGGAGTGGGAGAAGTAGAGATGGGGTTACTGTTTGTCTGTAGTTCCTGGAGCAGGAGGGCTGCCGGC
TCCCCCGTCCAGCTGAGGACCCTGCTATCTCCTGCCCCTGCCACCCTCCCTGCCAGCCCCAGGATTCTGC
TGCAGGCTGCTGTGGTTGTCCCGTTGCAGACATGAGTTCCACAGGGCCGAGTGTGGGACTTGAGTATGCA
TGGATTTTGGTATATGTCGGGGTCCTGGAACCCATCCCCCTGTAGCACTTCCCTGTGCCCTGGAGGTACT
GCCTGCCTCCCGACTTCCACCCTCACTTGCCATTACAGGTTTCCCATGACAGTGGGGGAGCCCAGGCCCC
AGGCAGTGCTACATTTGGATGTGGAGCCTCTCCTTTCCATTGTGAACTGAATTCTACCAGCACTGGGCCT
GGTGATGTCTGCATTAGGCAGATGAGGAAGCAGTCTTGGAGAGGACAAGGGACTGGCCTCAGGTCACACA
TCAGGCTTGCTAGCTCCTGGACAAAGATGCCTCTGATAGCTCTTCCATATTTCGCCCCTGCTGAGAGCTG
AGGGGAGGAGTGCGGCCACTAGCTGACCTGCCTGGGTCCCTGGGAGGCTGGTTTTGGGGAGGTAGTGATA
CATGTGCTCATGGTAGGGCTTGGGCAAGGGCCAGGAGAGCAGAGCCTGGGCCTGACCTTTCACCCCTGGT
TCCCAGAGCTGCCCACGGGAAGCCCCCAGGGCAGTAGGCAGTGCTTCCCTGCCCACAGTGCAAATCCCCC
TGCCTACTGCCAAAGCAGGAAGAGGAAGTGGCCGGGTCAGGGCTGGCAATAGGCCCTGGGGCCGAGGAGG
GGAAGCTCCCAGGGCCACTTCTGAGCTGGAGAAGGCAAAGCTCACTCCTTCTGCCCACCCCAGGGGAAGG
GTGTGGAGGCCCATGGGTGGGGACAGTCTCAGTGAAAGTAGGGCAAGTTCCTGACCTCTACTCAGTAGGG
GTGGGGCCCAGCCCAAGGTGGAGGGGCAGCGTGGGAAAAGGGCTGTGTAATTCCACTTCCTTCATCCCCT
GTTTACCCATCTCCACCCCTACCCTCTGGGCTGGGGAATGGTGGTGGACATTGGCTGCTCACTTGGGGCC
TGCCCTGGGCTACTTACTACTGAGGATGCACAAAGGATCTTCCCACCACGGACTAGGAGACAGTGCCCTT
GTCCTCATGTTGTTCACATACTGGTCGGTGAGGGATGGGTCTGAGTACATGATAAATCCCTGGGCCCCAT
AAGATTTGACTAGGAGGGGTAAGGGCCTGGTGAACTGCTCAGAAAATAAACTGTGTTGTCAGCAGGAGGA
CCCCTTTCTACTATGAGATATTGCATTCCCTGCTAATCATACTTGTGCTCTATCTGTTGATAGAACAAAT
ACATAATGAGAAACAGCCACTGTGCTTTCAGAAGAACACACTTGAAAGAATTTGTTCTCAATAAATTACA
TCTGCATCTGGATCCATATGAAGAAATACGTTCCTCTCATTCAGGCGTGGCTATTTTAAAATAAAATTTG
GGCCGGGCACGGTGACTCACGCCTGTAATCCCAGCACTTTGGGAGTCCGAGGCAGGCGGATCACGAGGTC
AGGAGATCGAGACCATCCTGGCTAACACAGTGAAACCCCATCTCTACTAAAAATACAAAAAATTAGCCGG
GCGTGGTGGCGGGCGCTTGTAGTCCCAGCTACTCAGGAGGCTACTAGGAGGAAGGGAAACAGTATGCACA
GTGGGTGAAACAGTATGTGCAAAGTGGGTGGTGGGAAGGTTTAGCTCAAGGTCCTTATATGGGGTCGGTG
GGGAACTGCGGTGATGAGGGCAGATGGATAGGCTGGGCCGTGGTGTGGGGGCCTGACTAGGTGGGGCTGT
CTCGTCTCCATCGTGACATCTCTCCCCGCCCTTCCATGTTGTCATGGCCTTGCCTTACCTCCAGGTCCCT
GTCTCTAGTTTTGGATACCTCTCTCATTATTACCTGTTCCCTCCTACCCCATCTCCCCGACAATTAGCTG
TTCACTCTGACCTTGTTCTTGAAGGTTAGACTTGGAGGGGTGTGTGCTGGGAGTCTGTGGCCAGTCTGGG
ACCAGCATTTGTTTTAGGCCTCCCAAAGTCCACGTCAATTGTGCCTTTCTCCAAAGTTCCACTAGCTGGA
CCCCACTCTCTCAGAGGCCCAGGCCATTCTCCCTTGAGGGCCCACAAGGCCTGGGATCCCCAAGCCCCTT
TCACCTCTCTCGCCTTTATCTTAGGCTAGGTGCTCCACACGGGAACCTGCATTGGTGGCCCCAGCTGTGT
TTCCACAGAGGGACTTTGCCTTTTTCTCCTTATTTACTTATTTTCAGAATGTGGTGTGTTGAGCACCCCC
ACCATCTCCCACCCCTGCAAGCATGACCAGGTGGCAGCTGTAGTTCCTTATCCTCCTCCAGGCTTTGGGG
CAGCAGGACTGCAGCCTCCATAACCTGCAGCAGCTTGGGGAGGTGGGGCCTGGGCTGCAGGCCCAGATCT
GGAGGCTAGATGCTGAGATTCAGACCAGGGACCTGGACAGCTCCTCACCACACTCAGTTTTCCTTGGGCT
TATGGGTGGGCCTACCCCACAGGATGGTCTGTGCCACTTTTTACAAATGCCAAGTTGTGAGGAGGAGAGG
AGGGTGCCTGAGCTTGAGGCTGGTTCAACTCTCAAAGAGCTAGCAAGGCTGGGCCGAGGGCGGTGGCTCA
CGCCTGTAATCCCAGCACTTTGGGAGGCCAAGGCGGGTGGATCACGAGGTCAGGAGATCGAGACTATCCT
GGTTAACACGGTGAAACCCCGTCTCTACTAAAAATACAAAAAATTAGCTGGGTGTGGTGGCAGGCACCTG
TCGTCCCAGCTACTCAGGAGGCTGAGGCAGGAGAATGGCATGAACCTGGGAGGCGGAGCTTGCAGTGAGC
CGAGATCGCACCACTTCACTCTAGCCTGGGCGACAGCACCATCTCAAAAACAAAACAAAACAAAAACAAA
AAACAAAAAAAGAGCTAGCAAGGCTGAGATTGAGCACAGACTGGTCAAGCCACAGATAACGCACTTGTTC
TTGCTGGTTGGCAGGAAGAACAATGGCTTGGCTGATCATCTTTTGCTACCTATTAAAATTGGTTTTATTT
TGGCCGGGTACAGTGGCTCATGCCTGTAATCCCAGCACTTTGGGAGGCCAAGGCGGGCAGATCACAAGGT
CAGGAGATCAAGACCATCCTGGCTAACACAGTGAAACCCCGTCTCTGCTAAAAATACAAAAAAATTAGCT
GGCGTGGTGGCAGGCACCTGTAGTCCCAGTGACTCGGGAGGCTGAGGCAGGAGAATGGCGTGAACCCGGG
AGGCGGAGCTTGCAGTGAGCCGAGATAGTGCCACTGCACTCCAGCCTGGGTGACAGAGCGAGACTCCGTC
TTAAAAAAAAAAGTTAGTTTTATTTTCTAGGGAAGGCCCTGCTGGGGTGGAGGGAGACATCCTGGGGCAA
AGTGGGTGCTAGGTGAGGAAGTCAGCCTGTGACACAGCTGGGATCAGTTCCACCCAGCAAGACTTGCTGT
GTGCTCCTGAGCAAAGTGTAGCTCCTCTTGGAACCCTTGACTCCTAATCTGCAAAATGGGAGGACTTGAA
TTCTATATGTGGTTTTAAACCTTTTTTTTTTTTAAGCTGTGGAGTCATTTTCCACAAAAGGATTCCACAA
AATCTAAGCAAAATCTTACGGAAAAGCCTAAGAGAAAGAAAAGCAAAACTTTGCCTGAAATGAGGTAGGG
ATCCTGGATGGGTCCAATTGGCATCATCCTGCCCACACTTCACATTCAACCAGTGGCCTTGAAGGAAGAT
GGTTTGGAGCAACCCAGAGCTCTCTGCAGATGCATTCACGTTGAGACTGTATGCTGGGATGGAGGGTTCT
GGCAGGTTCTGACCAGATGACTGAATGCCATCAGCATTACAGTCACCCCTTGGTTTATGAAGGGGGTTGG
CTCCAGGACCCCAACTTGTACTGCGAATACTCAAGTCCCACTGTTGGCCCTGTGGAACCTGTAGATACTA
AAAGTCGGCCCTCTGTATATGTGGGTTTTTGGATCCATGTTGGATTGAAAAAAAATCCCCAAATCCCCAT
ATGAGTGGACCTACACAGTTCAAACGTGTTGTTCAAGGGTCAACTGTGTATGAAGACTGCTCCATCTGTG
GTGGACAGTGAGGCTTTAGAAAGAGGAGACAGGCAGGCTAATGGATGGTCTTCATGGGTGTGGGAAAGGA
GGAGTGGTCACTGGGCGCAGGCAGTTGGTCTGTGGGACCAGTGAGGCATGGGCTGTGGCTGGCAGTAGGA
AAGCGGGAGAGGCTGAGTGCAGTGGCTCATGCCTATAATCCCAGCACTTTGGGAAGCCAAGGCAAGAGGC
TTGAGCCTGGGAGGTCCAGGCTGCAGTAAGCCATAATCATGCCACTGCATTCTAGGCTGGGCAACAGAAC
AAGACCTTGTCTCAAAGACAACAAAACAAAACAAAAACGGGGAGAGGAAAGGACCCAAGAGATGTTCCAA
AAAAGAATTTCAGGAAACAGTGAAAAGTTGCAAATGGGGCATCAGGAGCCAGGAGCAGAGATTAGGTGGC
AGGCAGGGGGTGCAGAAATGACAAGTTCTGTCTGGAACAGATGAAATTTGAGGTGCTTGTGGGACCTTTG
AGTGGGCAGAAGCGGGGGCTGGAGACAAGGGTGTGGGAAAACTGTGATGGGGGTTGAGGGTGGAAATCGG
GCCGGCAGAGCCTCCGGGGCAGAGAAAGTCCTATGGGGTGACCAGATGAGTTGAGAACTGGGTGGGTGGG
TGCAGGCTCAATGGAAGCAGAAACTCATGGATATTGACTTACATGGGAGAAGGGGAATGGATGGGGGTCG
CAGTGGGGTGGCAGGGCTCTCTTTTCCTTGTTTTTGTTTTTTTGCCCTTCCCTCTTCTCTTACTTTTCCG
TGAGGGCTCCCCAGGCTCAGGAGAGATCAGGGTGGAAGGGTGACGGCCAAACCAGGGAAGGCTCCAGGTG
GCTGAAGCCTGGTCTGTGCCCAGCAGGGCCCTGGCGGGCTGTTCCTCACTCCACCGGGCAGGTGATAACT
GGTCAGAGCTGCCTCCCCACAGGTGCTGGAGGTAGGCTGGGAGGGCCGGTGCTCCCTGATGTGGACAGGG
GGAGGGGTATTGATAAGAGGCGTGGAGAGATCCCTAGAGATGCAGTCTGTGGCCCTGGGTTCCAACCCAG
TGTGCCACCACCTGGCTGTGTGACCTTCAGCAAGTGCCATTATTTCTCTGAGCCTGTTTGTTTATAAAAT
GAGGAAGAGTTGGCACAAGTTTGAAAAGATTTCTCAGGCTCCACCCGGTTCTGAAATTCGGTAATTTCCC
AACTAGGGTGCACTCCCCCTGTAAGGGGCTGGGGAGGGGACGGTGCGAAACCAAGTTCAGCTTGTGGAGC
GGAGCCAGAGCTGTTGGCCGAGCTTGGGCCTGGCCAACGCCTGCCCTCGGGAAGGTCCTGTGTAGGGAGA
CTGCCTGGAGGGACTAAGCGAGGGCTCTAACTGACGTCTCAGGGGCAGCCTCTCAGCCTGAGACCCTGCT
GGGGAAGCCGCGTCCTGTGCACTAGCTGCGCCTAGGGCTGAGGTGAGGGCGCAGGCTCCCCAGGGTGTGT
CCGAATTGCCCGCCTCAGCCCGCGGCCTGTCCTGACCGCCCAGCAGCGGCAGCAGCGACACCCTAGGGCT
CCAGGAAGGGCTTGGGAAGGTGTAAAGGCGGGGCTAGGCTCGGAGGGAGCCGGAGGGACCGGGCGCGGTT
GGCTCCCGGGAGCAACTGGAGAGTGAGGAGATCCTCATCCGGGGAAGCCCCGCGGCCGCGTCTCTACAGC
GCCCCTTCTCGGGCTCTGGCCCTCCCGCTGGTTATTCTGGACCTGGGGGCCCCCAGCTGGGACCCGAGTC
CGGTGCGGGGAGCCTAGTGGGCCTGGGAGCTGCTATTTTTAGCGGGCGCGGCGGGCGCGAGGAGCCTATT
TATAGATCAAACAATCCGCGCTCCCTGCGTCAATGGAACCCCGCGTGCGTCACGCGCGCAGACATTCCAG
GCCCCCCCTCCTCGCCCCGCCCCCTCGGGCTCCCCGGGCCGCACCTCCCCCTGGCCGCCTCCCGCCGGAA
CCGCACCGCCCCCCGCGCCCTTGTATGGCCAAAGCTCGACGGGCGGCCTGCGTCAGTGGCGCCCCCGCCC
CTCCCCGTGCGTCACGGAGCGCTTAAGAGGAGGGTCGGGCTCGGCCGGGGAGTCCCAGTGGCGGAGGCTA
CGAAACTTGGGGGAGTGCACAGAAGAACTTCGGGAGCGCACGCGGGACCAGGGACCAGGCTGAGACTCGG
GGCGCCAGTCCGGGCAGGGGCAGCGGGAGCCGGCCGGGTAGGTTCCCTTCGGGGAACGTGCATCTGTTTT
TAGGAGCGGTGCATGAAGGAGATGGGTGTACGCGCGGGCAGAGAGGATGTTGTAGGGCCGGCATGCAAGA
GGGTAGGTGTAGTGTGCAGCTGTAGCACAACGGGATGAGAACCCAGGTCAGACTGGAAACTACTGGGTGC
GGGGTAGGAGGTAGGGGAGGTTGACTACCTGCAAAGTGGAAGCTGTAGGGGGTTGGGGTTGGCGCCGGGA
GTAGGGACTTGTCCAGCAGGCGGCTGGCCTTAGTTCAGTCTCCTAGGGTGCCCAACTGGGCGCACGGTGC
TCTGAACTTGGGTGGGTGTGGATGGGGGTCACAGCACCGTGGGGGAACAGGTACACAGAGCTTTGGCGCT
GCTGGGATCCGTACGATTCTAACTGGGATTGGATTTCCAAAAGGCCCTCCTTCCTCCCAGCCTGTCAGCT
CCCTCCCAGTGTCCCGGTTGTTTTCTTAACACTTTAGAGGCTGTGGCTGAGTTGGCAGGGGTTCCCCACC
CTGAACTTTCTGTCTGAGGTGCGTTAGGAAGCCTCCGGATCATTGAAGGAAGTGACTGGCTTGGTGGCTG
ACCTGTGGAGGGCTGCTTGCTGTGTATTGAATAAAGGAGGCTTCTTGGGGTCGGGGCAGGGTCTCTGTAA
GCACAGGGAGAATTGATTTCGCTGGGGCTTGGATCACGTGTGTGTGTGTGTGTGTGTGTGTGTGTGTGTG
TGTGAGAGAGAGAGAGAGAGAGAGAGAAAGAGAGACAGAGGTGGAAACCCTTGTCCTTGGGGATCCTGGA
TAGAAGTTGCCAGTGTTGGAGTCTTGGGGCGGTGCTGCCTAGAGGATACCTCCCAACCATTCCAGCTCCA
GATGCTGATCATCCCTATGCCCCCTCCCCCAGTAGTCTCTGTTGGATCTTACAGGTAGGGTGCAGCCTGA
GGCTTGTTCAGCAGAACAGGTGCAAGCCACATTGTTGCCAAGACCTGCCTGAAGCCGGATTCTCCCCACT
GCCTCCTTCAACCCCGCCTCTTCCTCCTCCTGTGGGACTGCTCCCCCCTCCTGTGAGGCTAGATGTAGGT
CCATATCTTGTGTTGTTAAGAACCTGCATGAAGGGGGAGGGACTGATGGGGGTTGATCCGGATGTGGGAC
GTCCAGGTGGAGAAACAGGATTTGAATAAGGCAGGAACAAGCGCCCAGTTCTGCCCAACTGCTGTCTTTC
CTCCTTCACCCCCAGCCCATCCCTGGCCAGGCTGTCCTAGTGCAGGCAGATGTGGCCACCCTCACCTCTC
GCAGGGTGCTGCATGCCTGTGGACTGGTGCCACTGGGTGTGTGGGGCCTGAGCTACAAGGGTCTCTGTTG
TCTTGGTTGTCAGGCTAGGGGTGGAGGTGCCCTGTTCAGGGCCTGTGGAGTTAGAAGGAAGGCTAGGACC
AGAGAGGACTTCCTGGGCTCTTGGCCAGGAGGTGGTGACAGCCTTTGCCCCCATCACTGCACCCCTTGAT
TATGGTACTCTGTTCCTGCTTGATTTCTTCTCCAGCTTCCCTCATTGACTGGTGAGGCCAGAGTTACGCA
GACCCCAGGTGTGTAGGTTGGGAGGGAGACTGTCCTGGAGCCTCCTCTGCATTATTTTTTGACCATTCTG
TGAATGTAAGGATCTGCCTGAATGATGCTATCTCTGTTCAGGCTCTCTTCAGGCTCCTCGGGACTGGGGC
TGTGTAACCCCCTTTGCCTGAGCCTCTGGATTCTTCAGGGCACAGACCCTGCTTTAGTGGAGGGACCCAG
GCAGGGGCAGAATGTCTTCCACCCACCCACCAGCAGGCTTCTCCTTAAGTCTTCTGCTGGACTCTCAGAG
TTCTCTTATTAAGATAACTGGGGGCAGCAGAACATTCTTTTTAGGTACTGTTGTCCATTCTCCTGAGAGT
CCAGGGGGCTGTGGTCTCCTTCCTCCCCCCTTGCCCTGTCCCCAGCAAATGGCCTTTAAAGGTCTGGAGC
CAGGTTACCGATGAGGAGGCCTAGGTTCCCTTCCTCTTTGCTTCTGGGAGCATCTCGAGCAGTGCAGAGT
GGCTTCCCAGCCCCAGATGGCAGGATTGGGGTGGGTTTGGGATCTGCTGCCTTTGCTCAGTGTGCAGGGT
TGGGGTGGAAATGGGGGACAGGCTAGGGCCTCTGCATTAGGCTGCCTTCTTAGGCAGGTGGGCTTTCACT
TCCAGCTCCTCTTCCATTTTCACGATGTCCTTCTTCCTAGGCACTGCGAGGGGAGGAGAAAAGGGGCTTT
GCAGAGGCCTGGGAGTATTCCCAGGAAGTGCCTGGTTGGCGAAACAACCAGGGAGCTGCTCCTGGGAGCT
GGGCTGAGGCTTTGCTGGGCTGCCTCTCCCACTCACCCTTCTCCCGGCCCCCACCATGCCTTCCCCATGG
GGGAGGGGCAGGGGGCTGGAGGAACACAGCTTCCCCCTGTTCTAGCAGAGAAATGCTGGCTGTATGCCTC
CCCTAGGGTTCCCAGGCTGACTAGGGTGTGGCTGGCCTTCTGATGGAGCCCACTCATGCTGGGCCGCTGC
CCAGGGGCTTTGTGGCACCTAGGTCGAGATGGTACTCAGGCCAGGGGTCAGGATTCCTGGGTGCTCTGGT
CCCGGTGCCTCTGTCTCATCTTTAGGCTGGGATTCCTGCCACCTTGCTGCTCTGGGGCCCAAATACTTTG
AGACAAGGCTATAGGCTTGTCCCACTGACTCTCCTTTCCCTCCCTGGGGTCTCCTCTCTCTCCAGAGATG
CCCTGTATCCAAGCCCAATATGGGACACCAGCACCGAGTCCGGGACCCCGTGACCACCTGGCAAGCGACC
CCCTGACCCCTGAGTTCATCAAGCCCACCATGGACCTGGCCAGCCCCGAGGCAGCCCCCGCTGCCCCCAC
TGCCCTGCCCAGCTTCAGCACCTTCATGGACGGCTACACAGGAGAGTTTGACACCTTCCTCTACCAGCTG
CCAGGAACAGTCCAGCCATGCTCCTCAGCCTCCTCCTCGGCCTCCTCCACATCCTCGTCCTCAGCCACCT
CCCCTGCCTCTGCCTCCTTCAAGTTCGAGGACTTCCAGGTGTACGGCTGCTACCCCGGCCCCCTGAGCGG
CCCAGTGGATGAGGCCCTGTCCTCCAGTGGCTCTGACTACTATGGCAGCCCCTGCTCGGCCCCGTCGCCC
TCCACGCCCAGCTTCCAGCCGCCCCAGCTCTCTCCCTGGGATGGCTCCTTCGGCCACTTCTCGCCCAGCC
AGACTTACGAAGGCCTGCGGGCATGGACAGAGCAGCTGCCCAAAGCCTCTGGGCCCCCACAGCCTCCAGC
CTTCTTTTCCTTCAGTCCTCCCACCGGCCCCAGCCCCAGCCTGGCCCAGAGCCCCCTGAAGTTGTTCCCC
TCACAGGCCACCCACCAGCTGGGGGAGGGAGAGAGCTATTCCATGCCTACGGCCTTCCCAGGTTTGGCAC
CCACTTCTCCACACCTTGAGGGCTCGGGGATACTGGATACACCCGTGACCTCAACCAAGGCCCGGAGCGG
GGCCCCAGGTGGAAGTGAAGGCCGCTGTGCTGTGTGTGGGGACAACGCTTCATGCCAGCATTATGGTGTC
CGCACATGTGAGGGCTGCAAGGGCTTCTTCAAGGTACCGCGCAGCCCCAGGTGGGGCCTTTTGTTGGAAA
TGGAGAGAGGCTGGCCTCATCCCATTGGGACCTGTGGTCTCCCCCTGGGTTCTCCTCCTAGCTAAGTCCT
GTCCTGCAGGGTGGGATCAGCCCTGCCAGGTGGGCCGCCTTCCTGGAGACCCGTAGATGCCAGGGCTGGA
AGCTTTCATTTGCCGGGACACTCGGGCCCATGGGATTGCACAGAGCTGGAGGGAGGGGTGAGATAGGGGC
AGATAGGAGCTGCAGGGGTGCCTGGCGAGCCTCTGGTTTTCCTCTGCTCCTCTGCCTGTCCTCTCCCAAC
TCAAGGTTCTAGTGGGAAGGGGTGCCCCCAGGCTCTCATGTTCCTGGCGTGAGATGAAAGGATCCCTGCG
GAGGGTTTGGTTCTTGAGGGCTGGGGGTGGACTTGGGAACAGGCTGTGTGTTTGTCCCAGCGATGGTGCC
TGCTTAGCTTCCCGTCCCCACCCCCCAGCCCCTTGGCCCTCTCCTGTCTGCCCTAGGGAGAAGGCAGGTG
GACAAGGGCCCATGAAAAAATACAGGTGTCTAGACTGCCAGGGAGACCCTGGCCCCCAGTAGTGTGTCCT
GGGGACTTCCTCAGAGCGAGAAACCTCCCCCAATGTCTTCAAGACTTTTCTCTCCCCCCGCCCAACCCCG
TCTCTCCCTCCCTTGCCACCCAAATGTTAGAAAAATAGCTGTGAACAGAGAGCGCTTTTGTCTGCAATGG
CAGCAGGATCTGGACGGTCCCCTCCCCTAAGTTCCCCCCTCCCCACCCCACACTCTGACAGCTTGTTCCG
TGTTGCCCCCCCACCCAGCGCACAGTGCAGAAAAACGCCAAGTACATCTGCCTGGCTAACAAGGACTGCC
CTGTGGACAAGAGGCGGCGAAACCGCTGCCAGTTCTGCCGCTTCCAGAAGTGCCTGGCGGTGGGCATGGT
GAAGGAAGGTGTGTGGCTGGGGTGCGGCCCAGCGGGGCAAGGGTAGGCTTGAGTGGAGTGGGACCAGCAG
GGCCCCCAGGCTTCTGCCCTGGAGGACCCAGAGGAGGGCACGTCTTATTTCCACCCCACCTCTGAACCCC
AGGCCTTGGAGGGAGGCAGCCTACACCTGCCTGGATTGTGAGGGTGGTGGCAGGGGGAGGTTCCTATAGG
GTACCTTGGATCTCAGGGACTCTGGGTCCTAGGGACTCGGTGGGGCGCGTCTCAGCAGTGGTGTGCACGG
CTTGGGCTGAGAGGCCCTTCCTCAGATCCCTTCCTTCCTCACCCCTACCCATTCCTTTGCAGTTGTCCGA
ACAGACAGCCTGAAGGGGCGGCGGGGCCGGCTACCTTCAAAACCCAAGCAGCCCCCAGATGCCTCCCCTG
CCAATCTCCTCACTTCCCTGGTCCGTGCACACCTGGACTCAGGGCCCAGCACTGCCAAACTGGACTACTC
CAAGGTGAGGTCCCACCCCGTGTCTGCCTTGGGGAGGTCTATGAGCACATGCAGTGCCTTTGTGCGTGTT
AGGAGAGCTACCCCCTCTGGAAGGACTGAATGAGAAAGGAGGTTTAAAAAAGAAAGAAAGAAAAGCGACT
CCCTCCAGTTCGACAGATCAAAGAGAGGATCCCCCTCTCGGCTGACCAGATGGGAAAATGCACCCCCTCA
GGCAGGTGGCCAATTAGAAAAATATGTCCTTTTGGCAGCTGCAGCCCTGGGTTAATATGTGAGACTTGGC
AAGTGAGAGCCTGGGCAGGATCTCAGATCCACTCCCACTCCCGGGATCTGGCATCCAAGTGTCTGACACA
GCCATACGTGGCAGTGGGTGTAGGAGCCTGCCTGGGGTGCTGACCCCACTGGACCGTCTTCCTAGTTCCA
GGAGCTGGTGCTGCCCCACTTTGGGAAGGAAGATGCTGGGGATGTACAGCAGTTCTACGACCTGCTCTCC
GGTTCTCTGGAGGTCATCCGCAAGTGGGCGGAGAAGATCCCTGGCTTTGCTGAGCTGTCACCGGCTGACC
AGGACCTGTTGCTGGAGTCGGCCTTCCTGGAGCTCTTCATCCTCCGCCTGGCGTACAGGTGAGAGCCACT
GACTGTCTGCCCAGCCCCTTTCCCTGATACACCTGCCTGTGAACCACCCTGATCGCTCTTCGTGCCCATC
TGCCTGCCAGGTCTAAGCCAGGCGAGGGCAAGCTCATCTTCTGCTCAGGCCTGGTGCTACACCGGCTGCA
GTGTGCCCGTGGCTTCGGGGACTGGATTGACAGTATCCTGGCCTTCTCAAGGTCCCTGCACAGCTTGCTT
GTCGATGTCCCTGCCTTCGCCTGCCTCTCTGCCCTTGTCCTCATCACCGGTGAGTGACCAGCACCACACC
AGGTCCAAGGGAATGGGCGTCAGGGGGTTGACTGGTTCTCAGGAGGGCACTGTCCCAGGGAGTTTGGTGG
GCCGGGATCTAGCACTTTCTGGGCCCCTGCACTGCCCCGGGCTCATGCCAGCAGTAAAGTAGGGACCATA
GGAATTGCAGATGGGCTCAGCTGCATCCAGCTCTAAAGCGCAAGACAGCCTTTGGTCCCAGACTGTCAGG
ATCCAGACTGCAGGTGTCATGTATGTGGCCTAGGGTATTGGCTTGTTTTTAAAACCTTCCTGTCAATTAT
GCTGCATATAGAAAAGTACACAAATGGTAAATTTTCATAAACCGCACTTACCCAACCAGCGCCTTGGTCA
AGAAAGAGCACATAACTGCCCCCCAGAACCCATTATTGTGCCCCCTTCCAGTCATTTGCCCTCCCCAAGG
GTAACCACTACTTTTTATTTAAATATTTTAAAAATAAGATGGGAATCTCACTATGTTGCCCGAGCTGGTC
TCGAACTCCTGGGCTCAAATGATCCTCCTGTCTCAGCCTCCTAAAGTGCTGGGATTACAGGTGTGAGCCA
CCACGCCTAGCCCTTCACTGTGACTTCTGACAGTGCAGATCAGATTGGTTGTGCCTGTTTTGGACTTTAT
GTAAATGTAGTTCTGCAGGATGGAATCTGGTGTTGAATGCAGAGGTTTTCAGATTTCTCTGTTTTTTAAA
GGAAAGAATCCACCCTCGTTCATTTTTTCACTTAAATTGCACAGGGGACCCAACGATATAGAACACAATC
AGAGGTACTCTGGGCTGAGGGAGTGCTGAGTTCTGAGGCTGGGTTTCTCAGAACAGTCTAGATTTTAAAA
ACCCAATGATCTAGCCAGAAAACGTAGGTTAGGATTTTATTTCCCGTTTGTGACCCTGGGCAAGTCATTA
GCCTCCTGGGCCTCGGGTTCTCACTTGGAGTATGAGGATAATGAGGGTTACTGCTTCTCAGACTTGTGAC
GATGCTTACTAATGGCCAACATGTGAATGCGCTTTTGTGAAGTGCCAGCAGAGCATGAGGGGTGGTCAGG
GGCAGCAGTTTTAGGGGCCTGGGGGAGGCTGGGGCTTTGGGGGCCTGGTTCTCAGATGTACAGCTAATCC
TGTACCCTTCCCGCAGACCGGCATGGGCTGCAGGAGCCGCGGCGGGTGGAGGAGCTGCAGAACCGCATCG
CCAGCTGCCTGAAGGAGCACGTGGCAGCTGTGGCGGGCGAGCCCCAGCCAGCCAGCTGCCTGTCACGTCT
GTTGGGCAAACTGCCCGAGCTGCGGACCCTGTGCACCCAGGGCCTGCAGCGCATCTTCTACCTCAAGCTG
GAGGACTTGGTGCCCCCTCCACCCATCATTGACAAGATCTTCATGGACACGCTGCCCTTCTGACCCCTGC
CTGGGAACACGTGTGCACATGCGCACTCTCATATGCCACCCCATGTGCCTTTAGTCCACGGACCCCCAGA
GCACCCCCAAGCCTGGGCTTGAGCTGCAGAATGACTCCACCTTCTCACCTGCTCCAGGAGGTTTGCAGGG
AGCTCAAGCCCTTGGGGAGGGGGATGCCTTCATGGGGGTGACCCCACGATTTGTCTTATCCCCCCCAGCC
TGGCCCCGGCCTTTATGTTTTTTGTAAGATAAACCGTTTTTAACACATAGCGCCGTGCTGTAAATAAGCC
CAGTGCTGCTGTAAATACAGGAAGAAAGAGCTTGAGGTGGGAGCGGGGCTGGGAGGAAGGGATGGGCCCC
GCCTTCCTGGGCAGCCTTTCCAGCCTCCTGCTGGCTCTCTCTTCCTACCCTCCTTCCACATGTACATAAA
CTGTCACTCTAGGAAGAAGACAAATGACAGATTCTGACATTTATATTTGTGTATTTTCCTGGATTTATAG
TATGTGACTTTTCTGATTAATATATTTAATATATTGAATAAAAAATAGACATGTAGTTGGAACTGA
SEQ ID NO: 2 (NM_002135.5, NR4A1 isoform 1 mRNA):
AGTCCCAGTGGCGGAGGCTACGAAACTTGGGGGAGTGCACAGAAGAACTTCGGGAGCGCACGCGGGACCA
GGGACCAGGCTGAGACTCGGGGCGCCAGTCCGGGCAGGGGCAGCGGGAGCCGGCCGGGTAGGGTGCAGCC
TGAGGCTTGTTCAGCAGAACAGGTGCAAGCCACATTGTTGCCAAGACCTGCCTGAAGCCGGATTCTCCCC
ACTGCCTCCTTCAACCCCGCCTCTTCCTCCTCCTGTGGGACTGCTCCCCCCTCCTGTGAGGCTAGATAGA
TGCCCTGTATCCAAGCCCAATATGGGACACCAGCACCGAGTCCGGGACCCCGTGACCACCTGGCAAGCGA
CCCCCTGACCCCTGAGTTCATCAAGCCCACCATGGACCTGGCCAGCCCCGAGGCAGCCCCCGCTGCCCCC
ACTGCCCTGCCCAGCTTCAGCACCTTCATGGACGGCTACACAGGAGAGTTTGACACCTTCCTCTACCAGC
TGCCAGGAACAGTCCAGCCATGCTCCTCAGCCTCCTCCTCGGCCTCCTCCACATCCTCGTCCTCAGCCAC
CTCCCCTGCCTCTGCCTCCTTCAAGTTCGAGGACTTCCAGGTGTACGGCTGCTACCCCGGCCCCCTGAGC
GGCCCAGTGGATGAGGCCCTGTCCTCCAGTGGCTCTGACTACTATGGCAGCCCCTGCTCGGCCCCGTCGC
CCTCCACGCCCAGCTTCCAGCCGCCCCAGCTCTCTCCCTGGGATGGCTCCTTCGGCCACTTCTCGCCCAG
CCAGACTTACGAAGGCCTGCGGGCATGGACAGAGCAGCTGCCCAAAGCCTCTGGGCCCCCACAGCCTCCA
GCCTTCTTTTCCTTCAGTCCTCCCACCGGCCCCAGCCCCAGCCTGGCCCAGAGCCCCCTGAAGTTGTTCC
CCTCACAGGCCACCCACCAGCTGGGGGAGGGAGAGAGCTATTCCATGCCTACGGCCTTCCCAGGTTTGGC
ACCCACTTCTCCACACCTTGAGGGCTCGGGGATACTGGATACACCCGTGACCTCAACCAAGGCCCGGAGC
GGGGCCCCAGGTGGAAGTGAAGGCCGCTGTGCTGTGTGTGGGGACAACGCTTCATGCCAGCATTATGGTG
TCCGCACATGTGAGGGCTGCAAGGGCTTCTTCAAGCGCACAGTGCAGAAAAACGCCAAGTACATCTGCCT
GGCTAACAAGGACTGCCCTGTGGACAAGAGGCGGCGAAACCGCTGCCAGTTCTGCCGCTTCCAGAAGTGC
CTGGCGGTGGGCATGGTGAAGGAAGTTGTCCGAACAGACAGCCTGAAGGGGCGGCGGGGCCGGCTACCTT
CAAAACCCAAGCAGCCCCCAGATGCCTCCCCTGCCAATCTCCTCACTTCCCTGGTCCGTGCACACCTGGA
CTCAGGGCCCAGCACTGCCAAACTGGACTACTCCAAGTTCCAGGAGCTGGTGCTGCCCCACTTTGGGAAG
GAAGATGCTGGGGATGTACAGCAGTTCTACGACCTGCTCTCCGGTTCTCTGGAGGTCATCCGCAAGTGGG
CGGAGAAGATCCCTGGCTTTGCTGAGCTGTCACCGGCTGACCAGGACCTGTTGCTGGAGTCGGCCTTCCT
GGAGCTCTTCATCCTCCGCCTGGCGTACAGGTCTAAGCCAGGCGAGGGCAAGCTCATCTTCTGCTCAGGC
CTGGTGCTACACCGGCTGCAGTGTGCCCGTGGCTTCGGGGACTGGATTGACAGTATCCTGGCCTTCTCAA
GGTCCCTGCACAGCTTGCTTGTCGATGTCCCTGCCTTCGCCTGCCTCTCTGCCCTTGTCCTCATCACCGA
CCGGCATGGGCTGCAGGAGCCGCGGCGGGTGGAGGAGCTGCAGAACCGCATCGCCAGCTGCCTGAAGGAG
CACGTGGCAGCTGTGGCGGGCGAGCCCCAGCCAGCCAGCTGCCTGTCACGTCTGTTGGGCAAACTGCCCG
AGCTGCGGACCCTGTGCACCCAGGGCCTGCAGCGCATCTTCTACCTCAAGCTGGAGGACTTGGTGCCCCC
TCCACCCATCATTGACAAGATCTTCATGGACACGCTGCCCTTCTGACCCCTGCCTGGGAACACGTGTGCA
CATGCGCACTCTCATATGCCACCCCATGTGCCTTTAGTCCACGGACCCCCAGAGCACCCCCAAGCCTGGG
CTTGAGCTGCAGAATGACTCCACCTTCTCACCTGCTCCAGGAGGTTTGCAGGGAGCTCAAGCCCTTGGGG
AGGGGGATGCCTTCATGGGGGTGACCCCACGATTTGTCTTATCCCCCCCAGCCTGGCCCCGGCCTTTATG
TTTTTTGTAAGATAAACCGTTTTTAACACATAGCGCCGTGCTGTAAATAAGCCCAGTGCTGCTGTAAATA
CAGGAAGAAAGAGCTTGAGGTGGGAGCGGGGCTGGGAGGAAGGGATGGGCCCCGCCTTCCTGGGCAGCCT
TTCCAGCCTCCTGCTGGCTCTCTCTTCCTACCCTCCTTCCACATGTACATAAACTGTCACTCTAGGAAGA
AGACAAATGACAGATTCTGACATTTATATTTGTGTATTTTCCTGGATTTATAGTATGTGACTTTTCTGAT
TAATATATTTAATATATTGAATAAAAAATAGACATGTAGTTGGAA
SEQ ID NO: 3 (NM_173157.3, NR4A1 isoform 1b mRNA):
AGTCCCAGTGGCGGAGGCTACGAAACTTGGGGGAGTGCACAGAAGAACTTCGGGAGCGCACGCGGGACCA
GGGACCAGGCTGAGACTCGGGGCGCCAGTCCGGGCAGGGGCAGCGGGAGCCGGCCGGAGATGCCCTGTAT
CCAAGCCCAATATGGGACACCAGCACCGAGTCCGGGACCCCGTGACCACCTGGCAAGCGACCCCCTGACC
CCTGAGTTCATCAAGCCCACCATGGACCTGGCCAGCCCCGAGGCAGCCCCCGCTGCCCCCACTGCCCTGC
CCAGCTTCAGCACCTTCATGGACGGCTACACAGGAGAGTTTGACACCTTCCTCTACCAGCTGCCAGGAAC
AGTCCAGCCATGCTCCTCAGCCTCCTCCTCGGCCTCCTCCACATCCTCGTCCTCAGCCACCTCCCCTGCC
TCTGCCTCCTTCAAGTTCGAGGACTTCCAGGTGTACGGCTGCTACCCCGGCCCCCTGAGCGGCCCAGTGG
ATGAGGCCCTGTCCTCCAGTGGCTCTGACTACTATGGCAGCCCCTGCTCGGCCCCGTCGCCCTCCACGCC
CAGCTTCCAGCCGCCCCAGCTCTCTCCCTGGGATGGCTCCTTCGGCCACTTCTCGCCCAGCCAGACTTAC
GAAGGCCTGCGGGCATGGACAGAGCAGCTGCCCAAAGCCTCTGGGCCCCCACAGCCTCCAGCCTTCTTTT
CCTTCAGTCCTCCCACCGGCCCCAGCCCCAGCCTGGCCCAGAGCCCCCTGAAGTTGTTCCCCTCACAGGC
CACCCACCAGCTGGGGGAGGGAGAGAGCTATTCCATGCCTACGGCCTTCCCAGGTTTGGCACCCACTTCT
CCACACCTTGAGGGCTCGGGGATACTGGATACACCCGTGACCTCAACCAAGGCCCGGAGCGGGGCCCCAG
GTGGAAGTGAAGGCCGCTGTGCTGTGTGTGGGGACAACGCTTCATGCCAGCATTATGGTGTCCGCACATG
TGAGGGCTGCAAGGGCTTCTTCAAGCGCACAGTGCAGAAAAACGCCAAGTACATCTGCCTGGCTAACAAG
GACTGCCCTGTGGACAAGAGGCGGCGAAACCGCTGCCAGTTCTGCCGCTTCCAGAAGTGCCTGGCGGTGG
GCATGGTGAAGGAAGTTGTCCGAACAGACAGCCTGAAGGGGCGGCGGGGCCGGCTACCTTCAAAACCCAA
GCAGCCCCCAGATGCCTCCCCTGCCAATCTCCTCACTTCCCTGGTCCGTGCACACCTGGACTCAGGGCCC
AGCACTGCCAAACTGGACTACTCCAAGTTCCAGGAGCTGGTGCTGCCCCACTTTGGGAAGGAAGATGCTG
GGGATGTACAGCAGTTCTACGACCTGCTCTCCGGTTCTCTGGAGGTCATCCGCAAGTGGGCGGAGAAGAT
CCCTGGCTTTGCTGAGCTGTCACCGGCTGACCAGGACCTGTTGCTGGAGTCGGCCTTCCTGGAGCTCTTC
ATCCTCCGCCTGGCGTACAGGTCTAAGCCAGGCGAGGGCAAGCTCATCTTCTGCTCAGGCCTGGTGCTAC
ACCGGCTGCAGTGTGCCCGTGGCTTCGGGGACTGGATTGACAGTATCCTGGCCTTCTCAAGGTCCCTGCA
CAGCTTGCTTGTCGATGTCCCTGCCTTCGCCTGCCTCTCTGCCCTTGTCCTCATCACCGACCGGCATGGG
CTGCAGGAGCCGCGGCGGGTGGAGGAGCTGCAGAACCGCATCGCCAGCTGCCTGAAGGAGCACGTGGCAG
CTGTGGCGGGCGAGCCCCAGCCAGCCAGCTGCCTGTCACGTCTGTTGGGCAAACTGCCCGAGCTGCGGAC
CCTGTGCACCCAGGGCCTGCAGCGCATCTTCTACCTCAAGCTGGAGGACTTGGTGCCCCCTCCACCCATC
ATTGACAAGATCTTCATGGACACGCTGCCCTTCTGACCCCTGCCTGGGAACACGTGTGCACATGCGCACT
CTCATATGCCACCCCATGTGCCTTTAGTCCACGGACCCCCAGAGCACCCCCAAGCCTGGGCTTGAGCTGC
AGAATGACTCCACCTTCTCACCTGCTCCAGGAGGTTTGCAGGGAGCTCAAGCCCTTGGGGAGGGGGATGC
CTTCATGGGGGTGACCCCACGATTTGTCTTATCCCCCCCAGCCTGGCCCCGGCCTTTATGTTTTTTGTAA
GATAAACCGTTTTTAACACATAGCGCCGTGCTGTAAATAAGCCCAGTGCTGCTGTAAATACAGGAAGAAA
GAGCTTGAGGTGGGAGCGGGGCTGGGAGGAAGGGATGGGCCCCGCCTTCCTGGGCAGCCTTTCCAGCCTC
CTGCTGGCTCTCTCTTCCTACCCTCCTTCCACATGTACATAAACTGTCACTCTAGGAAGAAGACAAATGA
CAGATTCTGACATTTATATTTGTGTATTTTCCTGGATTTATAGTATGTGACTTTTCTGATTAATATATTT
AATATATTGAATAAAAAATAGACATGTAGTTGGAA
SEQ ID NO: 4 (NM_001202233.2, NR4A1 isoform 2 mRNA):
TTCCTGGTGTAAGCTTTGGTATGGATGGTGGCCGTCTCCCTACAGACTGGGAGCTGTTAGAGGGCAGGGA
TCCTAGCTGACACATCTATGTCCTCGCCTTGGTTGGAGGCCTCCACCATGGACAGAGGCCAGGCCCTGCC
CCTCCCAGGCAGCCTGGCTCCTTCTGCTGGGCCCTGAAGGCAGACGGGATAATGTGGTTGGCCAAGGCCT
GTTGGTCCATCCAGAGTGAGATGCCCTGTATCCAAGCCCAATATGGGACACCAGCACCGAGTCCGGGACC
CCGTGACCACCTGGCAAGCGACCCCCTGACCCCTGAGTTCATCAAGCCCACCATGGACCTGGCCAGCCCC
GAGGCAGCCCCCGCTGCCCCCACTGCCCTGCCCAGCTTCAGCACCTTCATGGACGGCTACACAGGAGAGT
TTGACACCTTCCTCTACCAGCTGCCAGGAACAGTCCAGCCATGCTCCTCAGCCTCCTCCTCGGCCTCCTC
CACATCCTCGTCCTCAGCCACCTCCCCTGCCTCTGCCTCCTTCAAGTTCGAGGACTTCCAGGTGTACGGC
TGCTACCCCGGCCCCCTGAGCGGCCCAGTGGATGAGGCCCTGTCCTCCAGTGGCTCTGACTACTATGGCA
GCCCCTGCTCGGCCCCGTCGCCCTCCACGCCCAGCTTCCAGCCGCCCCAGCTCTCTCCCTGGGATGGCTC
CTTCGGCCACTTCTCGCCCAGCCAGACTTACGAAGGCCTGCGGGCATGGACAGAGCAGCTGCCCAAAGCC
TCTGGGCCCCCACAGCCTCCAGCCTTCTTTTCCTTCAGTCCTCCCACCGGCCCCAGCCCCAGCCTGGCCC
AGAGCCCCCTGAAGTTGTTCCCCTCACAGGCCACCCACCAGCTGGGGGAGGGAGAGAGCTATTCCATGCC
TACGGCCTTCCCAGGTTTGGCACCCACTTCTCCACACCTTGAGGGCTCGGGGATACTGGATACACCCGTG
ACCTCAACCAAGGCCCGGAGCGGGGCCCCAGGTGGAAGTGAAGGCCGCTGTGCTGTGTGTGGGGACAACG
CTTCATGCCAGCATTATGGTGTCCGCACATGTGAGGGCTGCAAGGGCTTCTTCAAGCGCACAGTGCAGAA
AAACGCCAAGTACATCTGCCTGGCTAACAAGGACTGCCCTGTGGACAAGAGGCGGCGAAACCGCTGCCAG
TTCTGCCGCTTCCAGAAGTGCCTGGCGGTGGGCATGGTGAAGGAAGTTGTCCGAACAGACAGCCTGAAGG
GGCGGCGGGGCCGGCTACCTTCAAAACCCAAGCAGCCCCCAGATGCCTCCCCTGCCAATCTCCTCACTTC
CCTGGTCCGTGCACACCTGGACTCAGGGCCCAGCACTGCCAAACTGGACTACTCCAAGTTCCAGGAGCTG
GTGCTGCCCCACTTTGGGAAGGAAGATGCTGGGGATGTACAGCAGTTCTACGACCTGCTCTCCGGTTCTC
TGGAGGTCATCCGCAAGTGGGCGGAGAAGATCCCTGGCTTTGCTGAGCTGTCACCGGCTGACCAGGACCT
GTTGCTGGAGTCGGCCTTCCTGGAGCTCTTCATCCTCCGCCTGGCGTACAGGTCTAAGCCAGGCGAGGGC
AAGCTCATCTTCTGCTCAGGCCTGGTGCTACACCGGCTGCAGTGTGCCCGTGGCTTCGGGGACTGGATTG
ACAGTATCCTGGCCTTCTCAAGGTCCCTGCACAGCTTGCTTGTCGATGTCCCTGCCTTCGCCTGCCTCTC
TGCCCTTGTCCTCATCACCGACCGGCATGGGCTGCAGGAGCCGCGGCGGGTGGAGGAGCTGCAGAACCGC
ATCGCCAGCTGCCTGAAGGAGCACGTGGCAGCTGTGGCGGGCGAGCCCCAGCCAGCCAGCTGCCTGTCAC
GTCTGTTGGGCAAACTGCCCGAGCTGCGGACCCTGTGCACCCAGGGCCTGCAGCGCATCTTCTACCTCAA
GCTGGAGGACTTGGTGCCCCCTCCACCCATCATTGACAAGATCTTCATGGACACGCTGCCCTTCTGACCC
CTGCCTGGGAACACGTGTGCACATGCGCACTCTCATATGCCACCCCATGTGCCTTTAGTCCACGGACCCC
CAGAGCACCCCCAAGCCTGGGCTTGAGCTGCAGAATGACTCCACCTTCTCACCTGCTCCAGGAGGTTTGC
AGGGAGCTCAAGCCCTTGGGGAGGGGGATGCCTTCATGGGGGTGACCCCACGATTTGTCTTATCCCCCCC
AGCCTGGCCCCGGCCTTTATGTTTTTTGTAAGATAAACCGTTTTTAACACATAGCGCCGTGCTGTAAATA
AGCCCAGTGCTGCTGTAAATACAGGAAGAAAGAGCTTGAGGTGGGAGCGGGGCTGGGAGGAAGGGATGGG
CCCCGCCTTCCTGGGCAGCCTTTCCAGCCTCCTGCTGGCTCTCTCTTCCTACCCTCCTTCCACATGTACA
TAAACTGTCACTCTAGGAAGAAGACAAATGACAGATTCTGACATTTATATTTGTGTATTTTCCTGGATTT
ATAGTATGTGACTTTTCTGATTAATATATTTAATATATTGAATAAAAAATAGACATGTAGTTGGAA
SEQ ID NO: 5 (NM_001202234.2, NR4A1 isoform 3 mRNA):
TTTCTCCGCCGGCTGCGGGAACCGGTTCCAGGAGCGCGGGAGCTCGTCTGCCAGGAGACCCAGCCGGGAG
GGACCGATGCTGCGTGATGCAACCCCTTCGAGGGACTGAGTCAAGAAACCAAGGCTGGGGGTCTCGTGGA
GTCAGGCGGAGCTGGGGATTAGGACTCGGGGCTTGTGTCTCCCAGGCGTGTTTTTTTGTTGTTGTTTTGT
TTTTTTGCCGCAACATCTTCCTTATACTGGGTGGGGTGGGGGCGGGGGTGGTCTGTATTTCCAGACCTCC
TCCATGGTGGTCTGGCTGTGTGGAGGGCAGGGCACAGTTCCTTGACTTGGCTCATTCCCCCCCAACGCCC
CCCCTTTCTTTTTTTTTTGAGATGGAGTCTCGCTCTGTTGCCCAGGCTGGAGTGCAGTAGCGTGATCTCG
GCTCACTGCAATCTCCGTCTCCCGGGTTCAAGCGAATCTCCTGCCTCAGCCTCCAGAGTAGCTGGGACTA
CAGGCACCCACCACCACGCCCGGCTAATTTTTGTATTTTTAGTAGAGACAGGGTTTCACCATATTGGCCA
GGCTGGTCTGGAACTCCTGACCTTGTGATCCGCCTGCCTCGGCCTCCTAAAGTGCTGGGATTACAGGCGT
GAGCCACCGCGCCTGGCAGGCTCATTCCCTTTTTATGCCTTCGTTTTTGATAGATGACCTGGGGATGGGA
GAAATGCTGCAGAGGTGGTTTATTTACAGTGAATTGTTAATTTGGTTGTGGGGCTAGTATTAGGGGCACA
TTGGGAAAACACAGGCTGGGCAGAGGTTGGTAGGTTCACAACAGTTATGCCCACTGGGTGCTGATGGATT
TGGGGGCTGTCGTCGGCCTGGCTTGGGGCCCTAGTGGTGGGGCTACAAGGCTTGCTCCTAGATCTACTCC
AAGTGAACATTTAAGATCAGTGACTTGGATGGAGACTTAAAAAGGAAGTCAGCTGACTCCGTGTAGTCAT
CCAGAGCTGTGAGGAATGACAAGTGCACAGTATAAAATCAAGATTCTAATTGAAGGCCTCCACCATGGAC
AGAGGCCAGGCCCTGCCCCTCCCAGGCAGCCTGGCTCCTTCTGCTGGGCCCTGAAGGCAGACGGGATAAT
GTGGTTGGCCAAGGCCTGTTGGTCCATCCAGAGTGAGATGCCCTGTATCCAAGCCCAATATGGGACACCA
GCACCGAGTCCGGGACCCCGTGACCACCTGGCAAGCGACCCCCTGACCCCTGAGTTCATCAAGCCCACCA
TGGACCTGGCCAGCCCCGAGGCAGCCCCCGCTGCCCCCACTGCCCTGCCCAGCTTCAGCACCTTCATGGA
CGGCTACACAGGAGAGTTTGACACCTTCCTCTACCAGCTGCCAGGAACAGTCCAGCCATGCTCCTCAGCC
TCCTCCTCGGCCTCCTCCACATCCTCGTCCTCAGCCACCTCCCCTGCCTCTGCCTCCTTCAAGTTCGAGG
ACTTCCAGGTGTACGGCTGCTACCCCGGCCCCCTGAGCGGCCCAGTGGATGAGGCCCTGTCCTCCAGTGG
CTCTGACTACTATGGCAGCCCCTGCTCGGCCCCGTCGCCCTCCACGCCCAGCTTCCAGCCGCCCCAGCTC
TCTCCCTGGGATGGCTCCTTCGGCCACTTCTCGCCCAGCCAGACTTACGAAGGCCTGCGGGCATGGACAG
AGCAGCTGCCCAAAGCCTCTGGGCCCCCACAGCCTCCAGCCTTCTTTTCCTTCAGTCCTCCCACCGGCCC
CAGCCCCAGCCTGGCCCAGAGCCCCCTGAAGTTGTTCCCCTCACAGGCCACCCACCAGCTGGGGGAGGGA
GAGAGCTATTCCATGCCTACGGCCTTCCCAGGTTTGGCACCCACTTCTCCACACCTTGAGGGCTCGGGGA
TACTGGATACACCCGTGACCTCAACCAAGGCCCGGAGCGGGGCCCCAGGTGGAAGTGAAGGCCGCTGTGC
TGTGTGTGGGGACAACGCTTCATGCCAGCATTATGGTGTCCGCACATGTGAGGGCTGCAAGGGCTTCTTC
AAGCGCACAGTGCAGAAAAACGCCAAGTACATCTGCCTGGCTAACAAGGACTGCCCTGTGGACAAGAGGC
GGCGAAACCGCTGCCAGTTCTGCCGCTTCCAGAAGTGCCTGGCGGTGGGCATGGTGAAGGAAGTTGTCCG
AACAGACAGCCTGAAGGGGCGGCGGGGCCGGCTACCTTCAAAACCCAAGCAGCCCCCAGATGCCTCCCCT
GCCAATCTCCTCACTTCCCTGGTCCGTGCACACCTGGACTCAGGGCCCAGCACTGCCAAACTGGACTACT
CCAAGTTCCAGGAGCTGGTGCTGCCCCACTTTGGGAAGGAAGATGCTGGGGATGTACAGCAGTTCTACGA
CCTGCTCTCCGGTTCTCTGGAGGTCATCCGCAAGTGGGCGGAGAAGATCCCTGGCTTTGCTGAGCTGTCA
CCGGCTGACCAGGACCTGTTGCTGGAGTCGGCCTTCCTGGAGCTCTTCATCCTCCGCCTGGCGTACAGGT
CTAAGCCAGGCGAGGGCAAGCTCATCTTCTGCTCAGGCCTGGTGCTACACCGGCTGCAGTGTGCCCGTGG
CTTCGGGGACTGGATTGACAGTATCCTGGCCTTCTCAAGGTCCCTGCACAGCTTGCTTGTCGATGTCCCT
GCCTTCGCCTGCCTCTCTGCCCTTGTCCTCATCACCGACCGGCATGGGCTGCAGGAGCCGCGGCGGGTGG
AGGAGCTGCAGAACCGCATCGCCAGCTGCCTGAAGGAGCACGTGGCAGCTGTGGCGGGCGAGCCCCAGCC
AGCCAGCTGCCTGTCACGTCTGTTGGGCAAACTGCCCGAGCTGCGGACCCTGTGCACCCAGGGCCTGCAG
CGCATCTTCTACCTCAAGCTGGAGGACTTGGTGCCCCCTCCACCCATCATTGACAAGATCTTCATGGACA
CGCTGCCCTTCTGACCCCTGCCTGGGAACACGTGTGCACATGCGCACTCTCATATGCCACCCCATGTGCC
TTTAGTCCACGGACCCCCAGAGCACCCCCAAGCCTGGGCTTGAGCTGCAGAATGACTCCACCTTCTCACC
TGCTCCAGGAGGTTTGCAGGGAGCTCAAGCCCTTGGGGAGGGGGATGCCTTCATGGGGGTGACCCCACGA
TTTGTCTTATCCCCCCCAGCCTGGCCCCGGCCTTTATGTTTTTTGTAAGATAAACCGTTTTTAACACATA
GCGCCGTGCTGTAAATAAGCCCAGTGCTGCTGTAAATACAGGAAGAAAGAGCTTGAGGTGGGAGCGGGGC
TGGGAGGAAGGGATGGGCCCCGCCTTCCTGGGCAGCCTTTCCAGCCTCCTGCTGGCTCTCTCTTCCTACC
CTCCTTCCACATGTACATAAACTGTCACTCTAGGAAGAAGACAAATGACAGATTCTGACATTTATATTTG
TGTATTTTCCTGGATTTATAGTATGTGACTTTTCTGATTAATATATTTAATATATTGAATAAAAAATAGA
CATGTAGTTGGAA
SEQ ID NO: 6 (NC_000002.12:c156332724-156324432 Homo sapiens chromosome 2,
GRCh38.p13 Primary Assembly, NR4A2 genomic sequence)
CGCAAGCCACATAAACAAAGGCACATTGGCGGCCAGGGCCAGTCCGCCCGGCGGCTCGCGCACGGCTCCG
CGGTCCCTTTTGCCTGTCCAGCCGGCCGCCTGTCCCTGCTCCCTCCCTCCGTGAGGTGTCCGGGTTCCCT
TCGCCCAGCTCTCCCACCCCTACCCGACCCCGGCGCCCGGGCTCCCAGAGGGAACTGCACTTCGGCAGAG
TTGAATGAATGAAGACAGACGCGGAGAACTCCTAAGTGAGTAGATGCAGCCCATGCAGTTCGCCTTCTTT
TATGCTTTTTCCTTCTTTTGCACGTCTCTTCTTTCCACTTGTGTGGGACAGGTTCTCTGGAAGTGGGAGC
CAGAGGCTTCTAGTGAGAGTGGGACCGAAGGATGGGGAGTGCGTGCGCGCAGTTACCGGGGGGCATTTGT
TCGAACTCCGGCTTTGGCACTAGTGGGGAGTTGGCTCTCGACAGAGGTTTCCAGGCTCCTCATTGGTGGA
CGTGGAAGGGAGACTCCACAGTTTGGGAGCTGAGGACTAGCCCGCGGAAATGTGCGCAAAGTTTGCTGTT
AGTGAGGAATTGATTGTGGCCTGTGAACACGGAGACTCCAAGTCCTATGTATACGAGGGAAGCTGCCCAC
AAACTGACAGGGAGAGGAAGTTCTTCAGTTTATGCGTTGCTTGGGAACTGTGTCTCCGCGGCTGGCCAGC
GCGCGATGTTTCCCGGGTATTGTTGAGTAAGGGTGGTGTTGGTAGCGTGTCCTGTTACTAAGTTGCCTGA
AATTTCTGGTTTTGACATATGCTGTCCTTGGGTTTGCGAATGTATCAGAGCGAGAATATGAATATGTAAA
GAGTACAGTTATGAAACTGTGGAGCTACCAGGGGGTTAATATCCAACACAGGAATATCTCTAAGGGCTGT
GGGGTTCGAGTCTCCTTTCTGCTTTTTCTGGGTAATCTTCCCCCCACCCCCACCACCACTATGAAGCAAG
ATTGTGGGGGAGGGGAAAGAATAAAAAGAGAGGATACTGGCTTTCTTTTTTCATTAGTAATAAGATTGTC
TGTGCTCTAGAATGTCTTCCAAGTGGGAACATCTGAAAATTGCTAGGACACAAGAATGCCTTGTTCCAGA
AAGGCAGATTGTGGAAGGCATTATGGGGAAGGTGTTCATCTTGCTGTGCTGGGAAACACTTCTAATATTG
GTGCCAATACCATATAAGCAGTATGTCCCCCCTCTGCAATTGACCTAAGAAGCTCCTGGAAAAGTAGATC
CCCTCTTCCCACCTTGTGACCATTAAGCCTTGTGACCATTAAAGATGCTGAAAGACAAGTTTTCTGGAAA
AGTGAACATCAATTTATCTGTAGCTCCAATCCCAGTGCTCTGTCAAAAGCACTTTAGAAGTGCGGATGCT
TCCACTCAACTTGCCTTCTCAGTCAAGGCCTTTCTAACATTTTGTAAGGGGGAAGATTGTTTTCTCATTT
TATATTCTTGACTTCTACTTTCTTCCCCTCTACCAAAAGAAAAGGCAATTTCACCACAAGAAAAAAAAAT
GCAAGAGAAGGTTCCAATGCTGTATTTTCATACTCTAGTCTTCATACTCAGGTCCTGAATTAACCTAAGA
TGGAAATGACCTCTCCACCTACACTGTAGCAAAGGGGCCAGTTCATTACATCATAAATGTTAAATGAGTT
CATGGACTAGCTTTCCTCTTGCAGGATCTTCTCTCTGCAAGGATTTACACAGTGCAATGGGTGGTATTTT
CTGTTGTTTCAAGTCATTTCTTTTATACATTCATTTTAAGTGCTATGTTTGGTAAAGGCTTCCCACTCAT
TTCCAATGAGACAAACAGGGAAGGCATGGAAGGGCCTGCCTGGTGAGTCTACATATGCCCAGCTGAATCT
CTGTCGGGAAGAAACCCTGAAGCTTCCTGTGTCTGTATTTCAGGGAGGAGATTGGACAGGCTGGACTCCC
CATTGCTTTTCTAAAAATCTTGGAAACTTTGTCCTTCATTGAATTACGACACTGTCCACCTTTAATTTCC
TCGAAAACGCCTGTAACTCGGCTGAAGGTTAGTGCAACTTCATTTCTTTCCTTTACTCTCCAGAGCTCCC
CAAACATCAAGAAACAGGACAAGGCAAACCCTGTAACTTAAGGTTTGCCCGACCCATCGCCTTCGGGAAC
AACTTTCTCATTGTGAAATTCAACTTCATTTCTAGATGGTCATTTCTAGAAAGAGACTGCTGAATCTGAG
CTTCAGAGAAGAGGCTCATCTGAGTGGGATGAGTGGGGGGGTATGAGGGAGATGTTTGGAAATACCCAGG
AGTGTAGACCCTCAGTAGCTTTTTAGCTCTGGGTCTTTATTTGGTTAGTCTTTCCACGCCCTAAACTGTT
GTTCTGCAGCATTCTCTCTCTCCTGCCTTTCCTCTCGCGCCCCTACATGCTCTCTGACTGCCGCGGGCTG
CCGGTGTAGCTCCAGGTGTACCCGAGCCCGGGAGAAAGTGTTCAGTTGACCAGGCTGAGTGTGTTATCAC
CCTGTTTCATTTCCAGCCATGCCTTGTGTTCAGGCGCAGTATGGGTCCTCGCCTCAAGGAGCCAGCCCCG
CTTCTCAGAGCTACAGTTACCACTCTTCGGGAGAATACAGCTCCGATTTCTTAACTCCAGAGTTTGTCAA
GTTTAGCATGGACCTCACCAACACTGAAATCACTGCCACCACTTCTCTCCCCAGCTTCAGTACCTTTATG
GACAACTACAGCACAGGCTACGACGTCAAGCCACCTTGCTTGTACCAAATGCCCCTGTCCGGACAGCAGT
CCTCCATTAAGGTAGAAGACATTCAGATGCACAACTACCAGCAACACAGCCACCTGCCCCCCCAGTCTGA
GGAGATGATGCCGCACTCCGGGTCGGTTTACTACAAGCCCTCCTCGCCCCCGACGCCCACCACCCCGGGC
TTCCAGGTGCAGCACAGCCCCATGTGGGACGACCCGGGATCTCTCCACAACTTCCACCAGAACTACGTGG
CCACTACGCACATGATCGAGCAGAGGAAAACGCCAGTCTCCCGCCTCTCCCTCTTCTCCTTTAAGCAATC
GCCCCCTGGCACCCCGGTGTCTAGTTGCCAGATGCGCTTCGACGGGCCCCTGCACGTCCCCATGAACCCG
GAGCCCGCCGGCAGCCACCACGTGGTGGACGGGCAGACCTTCGCTGTGCCCAACCCCATTCGCAAGCCCG
CGTCCATGGGCTTCCCGGGCCTGCAGATCGGCCACGCGTCTCAGCTGCTCGACACGCAGGTGCCCTCACC
GCCGTCGCGGGGCTCCCCCTCCAACGAGGGGCTGTGCGCTGTGTGTGGGGACAACGCGGCCTGCCAACAC
TACGGCGTGCGCACCTGTGAGGGCTGCAAAGGCTTCTTTAAGGTGAGCAATGGCGGGAGCGGAGTAGGCA
GGTAGGGAGCCCCTAGTGCCCGGGACCTCGGAGTGTGCCCTCTGCCTTGGTGCCAGTAGCCCAGCCCCAG
CTCTCCCGGGACTGCCCAGCTCTCCGGGGTCCGCCGAAGCTGCCCTGCAGGAGACCATGGGCTGCGGCGG
GGACTTCCGGGTGTCTGAGAAAGGGAAGCAGAAAGACTGGGAGGCCAGGGTCGCATCCCCCTCGCATTCA
GCCGACCCGGCTGGCCCCCGCCCGAAGTTGCTGGAGCCGGAGTTGGAAGAGGGTCATTTGCATGTGCTAG
GAGCTGTCTTCCCTGTTCAGAATGAAATTGGTTAGGACAGAGAACCGTGTCTGAGCTAACCAAGTGGAAC
AGAATTCCCTATGGTCAAATTAAGTGATCTCTTTATTTCGCCATCCTGATTGAATAATCTTATCATTTTA
AATAGAGAAGGTCTCCAAGGAATGTAAATAATATGAATGCCCACGGATTTGTATTTACTGAGCGTCTCCT
TCTCCTTCTCTTGGCATATAAAACACAGCAAGGAGCGGCAAGGTTAGCTCAAATGTTAACGCTATCAATT
TTCTTCTGTTAAATGCCCTGGGGGAGGAAAAAAGAAAAGAAAGAAAGAAAAGGAAGAGAAAAAAATAAAA
TGGAATTGTGTGTATGTGTTTGTTTGTGGGGAGGAATCGTAGACCCCAGTCACATAACAGAAATTTTCTC
CGAGTTGCCTGATTTTCAAAAGAAGAAAAAAAATGTTGGTCTATATTGTCTCCTTTTGCAGCGCACAGTG
CAAAAAAATGCAAAATACGTGTGTTTAGCAAATAAAAACTGCCCAGTGGACAAGCGTCGCCGGAATCGCT
GTCAGTACTGCCGATTTCAGAAGTGCCTGGCTGTTGGGATGGTCAAAGAAGGTAGGCTGAGGGGAGCTGC
CGACCCTCCAGTTTGCGCCTTTAGGAAACCACTGCTCATACTCCAGCATCACGTTCCACTTCCCGGTGCT
GGGGATCTCCGACTCCCCCTCAGTATGGCCTCCAGGACCCTGCAGCTGCCTGCTTGCCCGGCCTTCCCTA
GAGAAAGCCGCCAGGCCCTTCTCTCCTTTAACTATACGACCCATTTGGAGGAAGACATAAAATAACCCCG
CATTTTTTAATGCTTCTAGTCAGTGAAGGCTTTACAAGCACTGGGGCCCTCAGCCGCTCAGCCTGGTGCC
CCGCGGCTGCGGCCTTCCCCGGGGAGGGACCGAGGCAGCAGCTGGGCCTGGGCTCGGAAAAGCGGCGCTA
ACAGGGCTCTTCCTTTGCAGTGGTTCGCACAGACAGTTTAAAAGGCCGGAGAGGTCGTTTGCCCTCGAAA
CCGAAGAGCCCACAGGAGCCCTCTCCCCCTTCGCCCCCGGTGAGTCTGATCAGTGCCCTCGTCAGGGCCC
ATGTCGACTCCAACCCGGCTATGACCAGCCTGGACTATTCCAGGGTAAGAAGCTGGCGGGGGGGATATCA
TGTGGACAAACCGACAGATGGGCAGGACCCCTCCCCACATCCGTCATTAACTCTCAGATTCAACGGGGGT
AAAGAAGGCAAGCAAGGCTGTATATGCCTCGCAGCTCTGGCCAGGGCCTCAAGATTCAGATCTTCAGACA
ATCCATGTAGCTGGGGGCATAGACATGAGGACAGGATGGAGGAAGGAGGAGAGGGACACGCCACAGGGTT
TGAAGCTGTGTGAATTCCCACTACCCCACTACCCCATCGCCCCTCCTCTTCCATATACACCAGTGCCTCT
ACCATGAAATCCAGGGGCTGTGCAAACTCTCCCCCTTCCCAATCTACTTTATTCCCAGTCCTCCATAGAG
ATAGATGCTTTAATCCTCATCCTTCCTGGCACTGTGCTGGGGAAGGATGTGGGGGCCTGTCTGGGGGTCA
GGGAAGGGAAGGAGAGGGTGTAAAGAATGCCAGTGGGGTGGGGGATCAAGTGGTCAGATCCTTTTTACTC
CAGCTGTGAAAAATATGCGGGCTTTAATTGGAGGAAGTATGTTGAGCAAACCTGGTAGGGACTGCAATTT
TATTAAGATTTGCAAAAGGGCGTCTCAGCTCGAGGCCCACTCTGGGACTAGCATGAATACTAACATGTCA
ATTGTTTTGTGGAGATAAGAGTGAACGTTTCCCAGGGCTGGATGGCACTGTATTTAGTCTGTATGGAAAT
GGCAATTTACATATTTAAAGCAGCGACCTCGTAGCACCATCCCTAATTGAATTAATTGCCCCGGAACATC
TAATTTCCTTACTGGTCAGAGAGAGGTTTAATTGTTATAAAAACCTGGCTCCCCTATTAGAAACGGGGTT
AGCAATTTCACGGGTTATATATTTTAGAGAACCTCATTAAGTGCTTTTTAAAATGAAATTCCAGTTCCAG
GCGAACCCTGACTATCAAATGAGTGGAGATGACACCCAGCATATCCAGCAATTCTATGATCTCCTGACTG
GCTCCATGGAGATCATCCGGGGCTGGGCAGAGAAGATCCCTGGCTTCGCAGACCTGCCCAAAGCCGACCA
AGACCTGCTTTTTGAATCAGCTTTCTTAGAACTGTTTGTCCTTCGATTAGCATACAGGTAATAAGGGAGG
GAGGGAGACAATCCAGGGAGGCTGTGAGAGAAATCAAGAAAGGAAAAGAAAGGGAGGAAGGGAAACCAGA
GGGTGGGGTAGAGAAAAAGACAGAATAGGAAATGGAAGTCGGAGAAAGGAAGAAAAAGAAAGAAAACAAA
AAAAGACGAGAAGAAGCGAGCCCAGAAGCCTTGGATGAATGGAATGGAGGTGGGATAGGGGGCGTTCTTG
ATTGTTATGAAATTAAACCCTTTCAAGGTCCACTGGTCTACATTTTATTAACTCTTCAGTAATTAGGTGA
CTCTTAAATCCCTCATTTATTGCTCTTCAAGTAATTAGTTGTTTAGCTTTTCTCTCTCTCTTTTTCTCCC
CTCTCTCTCTTTGGTATTAATTGCAGGTCCAACCCAGTGGAGGGTAAACTCATCTTTTGCAATGGGGTGG
TCTTGCACAGGTTGCAATGCGTTCGTGGCTTTGGGGAATGGATTGATTCCATTGTTGAATTCTCCTCCAA
CTTGCAGAATATGAACATCGACATTTCTGCCTTCTCCTGCATTGCTGCCCTGGCTATGGTCACAGGTCAG
TACTGCAGGCGCAGGGCGCTTCCCCTCCAGAACTGCCTAGCAGGATTTGTCCTGAGTTTCCCTTGTCACA
GATTCTCCTTGGTTTTGCCAACTAGCTAACTGTCTTGTACATTCTTCTTTTGTTTCTGATTATGTTTTCT
GCAGAGAGACACGGGCTCAAGGAACCCAAGAGAGTGGAAGAACTGCAAAACAAGATTGTAAATTGTCTCA
AAGACCACGTGACTTTCAACAATGGGGGGTTGAACCGCCCCAATTATTTGTCCAAACTGTTGGGGAAGCT
CCCAGAACTTCGTACCCTTTGCACACAGGGGCTACAGCGCATTTTCTACCTGAAATTGGAAGACTTGGTG
CCACCGCCAGCAATAATTGACAAACTTTTCCTGGACACTTTACCTTTCTAAGACCTCCTCCCAAGCACTT
CAAAGGAACTGGAATGATAATGGAAACTGTCAAGAGGGGGCAAGTCACATGGGCAGAGATAGCCGTGTGA
GCAGTCTCAGCTCAAGCTGCCCCCCATTTCTGTAACCCTCCTAGCCCCCTTGATCCCTAAAGAAAACAAA
CAAACAAACAAAAACTGTTGCTATTTCCTAACCTGCAGGCAGAACCTGAAAGGGCATTTTGGCTCCGGGG
CATCCTGGATTTAGAACATGGACTACACACAATACAGTGGTATAAACTTTTTATTCTCAGTTTAAAAATC
AGTTTGTTGTTCAGAAGAAAGATTGCTATAATGTATAATGGGAAATGTTTGGCCATGCTTGGTTGTTGCA
GTTCAGACAAATGTAACACACACACACATACACACACACACACACACACAGAGACACATCTTAAGGGGAC
CCACAAGTATTGCCCTTTAACAAGACTTCAAAGTTTTCTGCTGTAAAGAAAGCTGTAATATATAGTAAAA
CTAAATGTTGCGTGGGTGGCATGAGTTGAAGAAGGCAAAGGCTTGTAAATTTACCCAATGCAGTTTGGCT
TTTTAAATTATTTTGTGCCTATTTATGAATAAATATTACAAATTCTAAAAGATAAGTGTGTTTGCAAAAA
AAAAGAAAATAAATACATAAAAAAGGGACAAGCATGTTGATTCTAGGTTGAAAATGTTATAGGCACTTGC
TACTTCAGTAATGTCTATATTATATAAATAGTATTTCAGACACTATGTAGTCTGTTAGATTTTATAAAGA
TTGGTAGTTATCTGAGCTTAAACATTTTCTCAATTGTAAAATAGGTGGGCACAAGTATTACACATCAGAA
AATCCTGACAAAAGGGACACATAGTGTTTGTAACACCGTCCAACATTCCTTGTTTGTAAGTGTTGTATGT
ACCGTTGATGTTGATAAAAAGAAAGTTTATATCTTGATTATTTTGTTGTCTAAAGCTAAACAAAACTTGC
ATGCAGCAGCTTTTGACTGTTTCCAGAGTGCTTATAATATACATAACTCCCTGGAAATAACTGAGCACTT
TGAATTTTTTTTATGTCTAAAATTGTCAGTTAATTTATTATTTTGTTTGAGTAAGAATTTTAATATTGCC
ATATTCTGTAGTATTTTTCTTTGTATATTTCTAGTATGGCACATGATATGAGTCACTGCCTTTTTTTCTA
TGGTGTATGACAGTTAGAGATGCTGATTTTTTTTCTGATAAATTCTTTCTTTGAGAAAGACAATTTTAAT
GTTTACAACAATAAACCATGTAAATGAACAGAA
SEQ ID NO: 7 (NM_006186.4, NR4A2 isoform a mRNA):
AAGCCACATAAACAAAGGCACATTGGCGGCCAGGGCCAGTCCGCCCGGCGGCTCGCGCACGGCTCCGCGG
TCCCTTTTGCCTGTCCAGCCGGCCGCCTGTCCCTGCTCCCTCCCTCCGTGAGGTGTCCGGGTTCCCTTCG
CCCAGCTCTCCCACCCCTACCCGACCCCGGCGCCCGGGCTCCCAGAGGGAACTGCACTTCGGCAGAGTTG
AATGAATGAAGACAGACGCGGAGAACTCCTAAGGAGGAGATTGGACAGGCTGGACTCCCCATTGCTTTTC
TAAAAATCTTGGAAACTTTGTCCTTCATTGAATTACGACACTGTCCACCTTTAATTTCCTCGAAAACGCC
TGTAACTCGGCTGAAGCCATGCCTTGTGTTCAGGCGCAGTATGGGTCCTCGCCTCAAGGAGCCAGCCCCG
CTTCTCAGAGCTACAGTTACCACTCTTCGGGAGAATACAGCTCCGATTTCTTAACTCCAGAGTTTGTCAA
GTTTAGCATGGACCTCACCAACACTGAAATCACTGCCACCACTTCTCTCCCCAGCTTCAGTACCTTTATG
GACAACTACAGCACAGGCTACGACGTCAAGCCACCTTGCTTGTACCAAATGCCCCTGTCCGGACAGCAGT
CCTCCATTAAGGTAGAAGACATTCAGATGCACAACTACCAGCAACACAGCCACCTGCCCCCCCAGTCTGA
GGAGATGATGCCGCACTCCGGGTCGGTTTACTACAAGCCCTCCTCGCCCCCGACGCCCACCACCCCGGGC
TTCCAGGTGCAGCACAGCCCCATGTGGGACGACCCGGGATCTCTCCACAACTTCCACCAGAACTACGTGG
CCACTACGCACATGATCGAGCAGAGGAAAACGCCAGTCTCCCGCCTCTCCCTCTTCTCCTTTAAGCAATC
GCCCCCTGGCACCCCGGTGTCTAGTTGCCAGATGCGCTTCGACGGGCCCCTGCACGTCCCCATGAACCCG
GAGCCCGCCGGCAGCCACCACGTGGTGGACGGGCAGACCTTCGCTGTGCCCAACCCCATTCGCAAGCCCG
CGTCCATGGGCTTCCCGGGCCTGCAGATCGGCCACGCGTCTCAGCTGCTCGACACGCAGGTGCCCTCACC
GCCGTCGCGGGGCTCCCCCTCCAACGAGGGGCTGTGCGCTGTGTGTGGGGACAACGCGGCCTGCCAACAC
TACGGCGTGCGCACCTGTGAGGGCTGCAAAGGCTTCTTTAAGCGCACAGTGCAAAAAAATGCAAAATACG
TGTGTTTAGCAAATAAAAACTGCCCAGTGGACAAGCGTCGCCGGAATCGCTGTCAGTACTGCCGATTTCA
GAAGTGCCTGGCTGTTGGGATGGTCAAAGAAGTGGTTCGCACAGACAGTTTAAAAGGCCGGAGAGGTCGT
TTGCCCTCGAAACCGAAGAGCCCACAGGAGCCCTCTCCCCCTTCGCCCCCGGTGAGTCTGATCAGTGCCC
TCGTCAGGGCCCATGTCGACTCCAACCCGGCTATGACCAGCCTGGACTATTCCAGGTTCCAGGCGAACCC
TGACTATCAAATGAGTGGAGATGACACCCAGCATATCCAGCAATTCTATGATCTCCTGACTGGCTCCATG
GAGATCATCCGGGGCTGGGCAGAGAAGATCCCTGGCTTCGCAGACCTGCCCAAAGCCGACCAAGACCTGC
TTTTTGAATCAGCTTTCTTAGAACTGTTTGTCCTTCGATTAGCATACAGGTCCAACCCAGTGGAGGGTAA
ACTCATCTTTTGCAATGGGGTGGTCTTGCACAGGTTGCAATGCGTTCGTGGCTTTGGGGAATGGATTGAT
TCCATTGTTGAATTCTCCTCCAACTTGCAGAATATGAACATCGACATTTCTGCCTTCTCCTGCATTGCTG
CCCTGGCTATGGTCACAGAGAGACACGGGCTCAAGGAACCCAAGAGAGTGGAAGAACTGCAAAACAAGAT
TGTAAATTGTCTCAAAGACCACGTGACTTTCAACAATGGGGGGTTGAACCGCCCCAATTATTTGTCCAAA
CTGTTGGGGAAGCTCCCAGAACTTCGTACCCTTTGCACACAGGGGCTACAGCGCATTTTCTACCTGAAAT
TGGAAGACTTGGTGCCACCGCCAGCAATAATTGACAAACTTTTCCTGGACACTTTACCTTTCTAAGACCT
CCTCCCAAGCACTTCAAAGGAACTGGAATGATAATGGAAACTGTCAAGAGGGGGCAAGTCACATGGGCAG
AGATAGCCGTGTGAGCAGTCTCAGCTCAAGCTGCCCCCCATTTCTGTAACCCTCCTAGCCCCCTTGATCC
CTAAAGAAAACAAACAAACAAACAAAAACTGTTGCTATTTCCTAACCTGCAGGCAGAACCTGAAAGGGCA
TTTTGGCTCCGGGGCATCCTGGATTTAGAACATGGACTACACACAATACAGTGGTATAAACTTTTTATTC
TCAGTTTAAAAATCAGTTTGTTGTTCAGAAGAAAGATTGCTATAATGTATAATGGGAAATGTTTGGCCAT
GCTTGGTTGTTGCAGTTCAGACAAATGTAACACACACACACATACACACACACACACACACACAGAGACA
CATCTTAAGGGGACCCACAAGTATTGCCCTTTAACAAGACTTCAAAGTTTTCTGCTGTAAAGAAAGCTGT
AATATATAGTAAAACTAAATGTTGCGTGGGTGGCATGAGTTGAAGAAGGCAAAGGCTTGTAAATTTACCC
AATGCAGTTTGGCTTTTTAAATTATTTTGTGCCTATTTATGAATAAATATTACAAATTCTAAAAGATAAG
TGTGTTTGCAAAAAAAAAGAAAATAAATACATAAAAAAGGGACAAGCATGTTGATTCTAGGTTGAAAATG
TTATAGGCACTTGCTACTTCAGTAATGTCTATATTATATAAATAGTATTTCAGACACTATGTAGTCTGTT
AGATTTTATAAAGATTGGTAGTTATCTGAGCTTAAACATTTTCTCAATTGTAAAATAGGTGGGCACAAGT
ATTACACATCAGAAAATCCTGACAAAAGGGACACATAGTGTTTGTAACACCGTCCAACATTCCTTGTTTG
TAAGTGTTGTATGTACCGTTGATGTTGATAAAAAGAAAGTTTATATCTTGATTATTTTGTTGTCTAAAGC
TAAACAAAACTTGCATGCAGCAGCTTTTGACTGTTTCCAGAGTGCTTATAATATACATAACTCCCTGGAA
ATAACTGAGCACTTTGAATTTTTTTTATGTCTAAAATTGTCAGTTAATTTATTATTTTGTTTGAGTAAGA
ATTTTAATATTGCCATATTCTGTAGTATTTTTCTTTGTATATTTCTAGTATGGCACATGATATGAGTCAC
TGCCTTTTTTTCTATGGTGTATGACAGTTAGAGATGCTGATTTTTTTTCTGATAAATTCTTTCTTTGAGA
AAGACAATTTTAATGTTTACAACAATAAACCATGTAAATGAA
SEQ ID NO: 8 (NM_173173.3, NR4A2 isoform d mRNA):
AAGCCACATAAACAAAGGCACATTGGCGGCCAGGGCCAGTCCGCCCGGCGGCTCGCGCACGGCTCCGCGG
TCCCTTTTGCCTGTCCAGCCGGCCGCCTGTCCCTGCTCCCTCCCTCCGTGAGGTGTCCGGGTTCCCTTCG
CCCAGCTCTCCCACCCCTACCCGACCCCGGCGCCCGGGCTCCCAGAGGGAACTGCACTTCGGCAGAGTTG
AATGAATGAAGACAGACGCGGAGAACTCCTAAGGAGGAGATTGGACAGGCTGGACTCCCCATTGCTTTTC
TAAAAATCTTGGAAACTTTGTCCTTCATTGAATTACGACACTGTCCACCTTTAATTTCCTCGAAAACGCC
TGTAACTCGGCTGAAGCTTCAGTACCTTTATGGACAACTACAGCACAGGCTACGACGTCAAGCCACCTTG
CTTGTACCAAATGCCCCTGTCCGGACAGCAGTCCTCCATTAAGGTAGAAGACATTCAGATGCACAACTAC
CAGCAACACAGCCACCTGCCCCCCCAGTCTGAGGAGATGATGCCGCACTCCGGGTCGGTTTACTACAAGC
CCTCCTCGCCCCCGACGCCCACCACCCCGGGCTTCCAGGTGCAGCACAGCCCCATGTGGGACGACCCGGG
ATCTCTCCACAACTTCCACCAGAACTACGTGGCCACTACGCACATGATCGAGCAGAGGAAAACGCCAGTC
TCCCGCCTCTCCCTCTTCTCCTTTAAGCAATCGCCCCCTGGCACCCCGGTGTCTAGTTGCCAGATGCGCT
TCGACGGGCCCCTGCACGTCCCCATGAACCCGGAGCCCGCCGGCAGCCACCACGTGGTGGACGGGCAGAC
CTTCGCTGTGCCCAACCCCATTCGCAAGCCCGCGTCCATGGGCTTCCCGGGCCTGCAGATCGGCCACGCG
TCTCAGCTGCTCGACACGCAGGTGCCCTCACCGCCGTCGCGGGGCTCCCCCTCCAACGAGGGGCTGTGCG
CTGTGTGTGGGGACAACGCGGCCTGCCAACACTACGGCGTGCGCACCTGTGAGGGCTGCAAAGGCTTCTT
TAAGCGCACAGTGCAAAAAAATGCAAAATACGTGTGTTTAGCAAATAAAAACTGCCCAGTGGACAAGCGT
CGCCGGAATCGCTGTCAGTACTGCCGATTTCAGAAGTGCCTGGCTGTTGGGATGGTCAAAGAAGTGGTTC
GCACAGACAGTTTAAAAGGCCGGAGAGGTCGTTTGCCCTCGAAACCGAAGAGCCCACAGGAGCCCTCTCC
CCCTTCGCCCCCGGTGAGTCTGATCAGTGCCCTCGTCAGGGCCCATGTCGACTCCAACCCGGCTATGACC
AGCCTGGACTATTCCAGGTTCCAGGCGAACCCTGACTATCAAATGAGTGGAGATGACACCCAGCATATCC
AGCAATTCTATGATCTCCTGACTGGCTCCATGGAGATCATCCGGGGCTGGGCAGAGAAGATCCCTGGCTT
CGCAGACCTGCCCAAAGCCGACCAAGACCTGCTTTTTGAATCAGCTTTCTTAGAACTGTTTGTCCTTCGA
TTAGCATACAGGTCCAACCCAGTGGAGGGTAAACTCATCTTTTGCAATGGGGTGGTCTTGCACAGGTTGC
AATGCGTTCGTGGCTTTGGGGAATGGATTGATTCCATTGTTGAATTCTCCTCCAACTTGCAGAATATGAA
CATCGACATTTCTGCCTTCTCCTGCATTGCTGCCCTGGCTATGGTCACAGAGAGACACGGGCTCAAGGAA
CCCAAGAGAGTGGAAGAACTGCAAAACAAGATTGTAAATTGTCTCAAAGACCACGTGACTTTCAACAATG
GGGGGTTGAACCGCCCCAATTATTTGTCCAAACTGTTGGGGAAGCTCCCAGAACTTCGTACCCTTTGCAC
ACAGGGGCTACAGCGCATTTTCTACCTGAAATTGGAAGACTTGGTGCCACCGCCAGCAATAATTGACAAA
CTTTTCCTGGACACTTTACCTTTCTAAGACCTCCTCCCAAGCACTTCAAAGGAACTGGAATGATAATGGA
AACTGTCAAGAGGGGGCAAGTCACATGGGCAGAGATAGCCGTGTGAGCAGTCTCAGCTCAAGCTGCCCCC
CATTTCTGTAACCCTCCTAGCCCCCTTGATCCCTAAAGAAAACAAACAAACAAACAAAAACTGTTGCTAT
TTCCTAACCTGCAGGCAGAACCTGAAAGGGCATTTTGGCTCCGGGGCATCCTGGATTTAGAACATGGACT
ACACACAATACAGTGGTATAAACTTTTTATTCTCAGTTTAAAAATCAGTTTGTTGTTCAGAAGAAAGATT
GCTATAATGTATAATGGGAAATGTTTGGCCATGCTTGGTTGTTGCAGTTCAGACAAATGTAACACACACA
CACATACACACACACACACACACACAGAGACACATCTTAAGGGGACCCACAAGTATTGCCCTTTAACAAG
ACTTCAAAGTTTTCTGCTGTAAAGAAAGCTGTAATATATAGTAAAACTAAATGTTGCGTGGGTGGCATGA
GTTGAAGAAGGCAAAGGCTTGTAAATTTACCCAATGCAGTTTGGCTTTTTAAATTATTTTGTGCCTATTT
ATGAATAAATATTACAAATTCTAAAAGATAAGTGTGTTTGCAAAAAAAAAGAAAATAAATACATAAAAAA
GGGACAAGCATGTTGATTCTAGGTTGAAAATGTTATAGGCACTTGCTACTTCAGTAATGTCTATATTATA
TAAATAGTATTTCAGACACTATGTAGTCTGTTAGATTTTATAAAGATTGGTAGTTATCTGAGCTTAAACA
TTTTCTCAATTGTAAAATAGGTGGGCACAAGTATTACACATCAGAAAATCCTGACAAAAGGGACACATAG
TGTTTGTAACACCGTCCAACATTCCTTGTTTGTAAGTGTTGTATGTACCGTTGATGTTGATAAAAAGAAA
GTTTATATCTTGATTATTTTGTTGTCTAAAGCTAAACAAAACTTGCATGCAGCAGCTTTTGACTGTTTCC
AGAGTGCTTATAATATACATAACTCCCTGGAAATAACTGAGCACTTTGAATTTTTTTTATGTCTAAAATT
GTCAGTTAATTTATTATTTTGTTTGAGTAAGAATTTTAATATTGCCATATTCTGTAGTATTTTTCTTTGT
ATATTTCTAGTATGGCACATGATATGAGTCACTGCCTTTTTTTCTATGGTGTATGACAGTTAGAGATGCT
GATTTTTTTTCTGATAAATTCTTTCTTTGAGAAAGACAATTTTAATGTTTACAACAATAAACCATGTAAA
TGAA
SEQ ID NO: 9 (NC_000009.12:99821885-99866893 Homo sapiens chromosome 9,
GRCh38.p13 Primary Assembly, NR4A3 genomic sequence):
GCGCAGCCGGGAGAGCGGAGTCTCCTGCCTCCCGCCCCCCACCCCTCCAGCTCCTGCTCCTCCTCCGCTC
CCCATACACAGACGCGCTCACACCCGCTCCCTCACTCGCACACACAGACACAAGCGCGCACACAGGCTCC
GCACACACACTTCGCTCTCCCGCGCGCTCACACCCCTCTTGCCCTGAGCCCTTGCCGGTGCAGCGCGGCG
CCGCAGCTGGACGCCCCTCCCGGGCTCACTTTGCAACGCTGACGGTGCCGGCAGTGGCCGTGGAGGTGGG
AACAGCGGCGGCATCCTCCCCCCTGGTCACAGCCCAAGCCAGGACGCCCGCGGAACCTCTCGGCTGTGCT
CTCCCATGAGTCGGGATCGCAGCATCCCCCACCAGCCGCTCACCGCCTCCGGGAGCCGCTGGGCTTGTAC
ACCGCAGCCCTTCCGGGACAGCAGCTGTGACTCCCCCCCAGTGCAGATTTCGGGACAGCTCTCTAGAAAC
TCGCTCTAAAGACGGAACCGCCACAGCACTCAAGTACGTATGAGATTCGCCCAGTTAATAAGGGGACTTG
CTAAAAAAGTTGGCTTGCTGGGAAGCGTCGCTCCTGAAATTGACAACTTTGAGTGTGGGGTTCCCTCCCC
TGACCTTGCCGCCCGGCTCCGGCTTTGCTAGCCCAGCGGCCCGTAGGTTCTGATCTGAAACTTTTCCCCT
GTAGTGGGCCCGCGGGGATCTCTGGAGCTCGTGGGATTCCTCCCCCCACTCGAAGAGGCGAAAAGCCTCT
AACAAAGAGGAGGACCGGGATTTGTGCTATAGCGGCTCCAGCGATGTAATTCAGGGTATTTCGGCTCTAG
TTGTCATGGTAATGATGCTCTCGGCGGCGGCGGCGGCGGCGGCAGCGGCAGCGGCAGGGAGTTGCAGCTC
CGGTGATGAACGGCAGTAATTTTCCTGCCTTTTAAGTAGGATTGAAAATAGGAGCTCTGGTGGGTCCAAG
TAAATGTTGCTAATGGTGGCACCGAGCTGGTTCTCTGGAAGGAAGCTTAGGAGGGAAGGGCCTAGGCAGC
GACGGCCAGAGTTTGCAGACGCACTCGGAGGACCTCAGGAAAGAGGGCGTAATTGTAGGAATGTGGCTTA
TTGTATTGAAATGAGCCTGGGGTTCCACTAGGCACGTCTGCCTGTCGTCGCTGACATGCCGGTTTACACT
TTTCCCTTAGGAGGTAAATCGGGTGTAATTGGCGACTCCCGCACACTGACACGTGTGGGGACGGTGTCCC
TCTCCTCTGGACGTTGGCTCGGTGTGGGAAAGGCATGCTTTTTCACGGACAGAACTCGCGCTTTTGGAGA
AGTTGCTCCGAGTGTTTTACTCTTAGTAGAAATGAAAGTTCTCGGTGGTGAGACGAGAGCGGTCATGCGA
GCGCAGCCTGCGGAGACTGACTTCCAAAGGCACCTCTCCAACCTGTTGGAAGCCTCGGGGCAGTGGTGAG
GAAGAACGCATTTGTCCCTTGATTTCCAAAAACGTTCTTGGAGATTGCCTTTTATTTATTTATGCGTCCG
GATTCTCCAGAGGGAAGAGCTGCGAGGAGTTAGTAATCAGAAATGTCGGGCTGTGATCCCAGGGCCTTGT
GGTGCTGGGAGAGAAGAACAGGGAGCCGTCATTCCATCGCGAGTCCAGTGCCGGATCTCCTTTTCCCACG
ACCGGGAAATATTTTATAGAATCAAGTTAGGGAAAGAAAAAGCTGCCTAACGTGGCTGATGGGCACAATG
AATGAAGTTACTTTATTCGAGTTATTTTCGCTGAACAAGATGCTCGCAGAGTTAGTATCAAAGCCTCCTT
AGGCCACAGCAACCTCCACGTCCGCTCCGCCCCCCACCCCCCGGCACCCATCCACCCCTTTGCCTGGGAC
CGTGCGCTCACAGAAATTCTTCCCCGCGGTTTGTCTAGTCTCCCTCGGTCTTTCTGAGAATCTCAGTCTT
TCCCTTTCTGTCAGTCTCTCCCTCGCTCCTCTTGCCCCCGCCCAACCTCCGCCCCCGCTCCCCAGGGTTG
GAAACTCCGCAGAGGAGCCCCGGTCACAATGGTGCGTTTCACGGTTTCCTCCACACACTTCACCACCGGG
AGGGGAACACGGGGGTGTTCCTTTCGTCTGCCCACGAGAAAGAAAGGGGCTTTGACAGAGGTAGTTATTT
CTTCCTAATTTACAACCCCAAGGCTCTGTGCTCCCGGCCCCGGCGGCCGCAAGCAGGAGCGGCGGAACGT
GTTTCCAGGCACTGAGGCTTTGCCTAGGTAACCGGCCGCTTGTGGGGTGGAGAGAGCGGCGGGGCGCTGG
GCTGGGCTGGAGGAAAGGCTGTGTGGGTCCCTCCCGCGAACGCTGGGCGCTCGAGGGGAACTCCTTCGTT
GGGGAGAGGCGGTCCCCGCCCACCCGAGTTCTGAGGCCTCCTCTCAGAGAACGACTCCGCTTGAAAGGCC
CTCAGGGAATGCGGGACGGGAATGCGGGCACCCACTGAAGGGGCGGGTGGGGGATTGCTGGAGACCCCCA
TTGAAGATGCGGGGAGACGGGAAGCACGTGCTACAGCCGTGGGAAGATCCGCTTCTACCCGCGCTACATC
GAGCGCAAGCTTTCCGTCCGAGCAGCTCCAGCTGGGGCTGGGGGTGGCAAGGGAATCCCCCATTTCCTGG
TCGTCGAGGGTTTCCTGGACCTGGGAGCCCCGAGTCCCTTTCTCTCTTCCTGCAGGCAAGCCCCGGGCTC
AGGGGCGCCAGGAGATGACCCTCGTGGCCCCGCGCACCTCGGAGGTTTTTGGTTTGGTTTGATTTGGGGT
TTTTCGGTCCCTGTTCCCACTCTTCGAGCTGCGCCCACCCCGGGCAAAGGGGGCGCTATAGGCCGGAGTT
TGGGCACCCAGTTCCTTCCCTGAGGCCTGCTGAGCTGCGCTTTCAGCTACAAAGTTTCGCTGAGGCTGTG
GGCTGAGACGCTGGTCCCGGAGCTGCGCTCGGCGCCCTCAGGAATGTGCCCCCGCTAGGCCGCTGGGTGG
CCAGGAGCCTCCAAGCCGCCCACCCTCGAAGACACCGCCCTCTGGGTGCAGGGGACCTGCCTCCGCTCGT
CCCATCAGCCGCTAACGCCGTCCGCTTCGTCCCCTTGCTTCCAGCGCCAGCCTTCTCATCGCTGTGCCCT
TTTGTTTGCTTGACCCCTGGCCCTCGAAACTCGCGGCTAATAGAAGCGAAGCTCCATTAGCATTTAGAAT
GAAAAGCGCAGACTTTCTTAATTCCTCGGGGCATTCATGCATTCGTTCCGGACCTTGTGCATTTCCTATG
CAACGTGTAAAATTTGTATTTGAGGGGTGGGGGCGGGCGGAGCTGGAAAATGGCTTTTTTCCGCTGGTGA
ACACTCACTGACCCCCCGTATTCGGGCGGAGCTCGTTCCTCAAGTGTTCTGCTTTCTTTGGTTTTCCTGA
CTTCGTAAGCTCGGAATCAATTGTGGGGGCAGAGAGATCAATTTCTGGGCAATAACAGGCTGCGCGTTTT
TTTTTTAGCCTCGGTTTTGGTGCCACAAAACGGTGGTAGGCGCAGTGGGTTCCAACAACCTTTCCTCGGC
TTCCCCGAGGCCGTGTGATGCTTCTTGCCCAGGGGGACTCCTGTACCATTAATCACCCTTAATTTTACGA
TAATTCTTTGTAATTAATGTAACATTTCTAGAGGTTGCCAACCAATTCCCCCAGTCCCCAATGCGCCCAG
CCCGGGCCGTAGTGACCCATCCTGGTCTCACTGTGACCTATGTCCTCTTTCACCTTGCCTGTAGAGCCCA
CTGCGGAAGAGGGCAGCCCGGCAAGCCCGGGCCCTGAGCCTGGACCCTTAGCGGTGCCGGGCAGCACTGC
CGGCGCTTCGCCTCGCCGGACGTCCGCTCCTCCTACACTCTCAGCCTCCGCTGGAGAGACCCCCAGCCCC
ACCATTCAGCGCGCAAGATACCCTCCAGGTAGGTCCGAAGGCAAGACCCTTTTCTCCTCCCTGGCTGAGG
GAAGTGGGTGGGGGAACCACACACTCGGCGGGCAGCGTGGTCGACCTGCCCAGTGCCAGGACAGTGACTG
CTGGCCGCGAATTTCACAACACAGGTGGCTTCCTCACAGGAAGCTCCTCTGTATACCACACCCTGTTGCT
ACTGAGTGGAGCAGCCAAATTAAATTAAGCTTGCATTGCTCAAAATTAATTTTCCTAAGAGAAATACAAA
TACACCAATAGATTAGGGTATTTTATACATTTTTAATTTCATTTTTGCTCTTCTTTTATAGCCAGTGTGC
ACATGTAAGAGTTATGGAATCACTTAGAGCAATACTGAGCATTTTCATTTATATAAAACCCAAATCATTT
GGGTGCAGAAGTTTGGATGATTGAAGCTCAGAGGGAAGGAGAAAAGCATTTGAGATGAAAACCAAAGAGT
AAATTTGAGTTTGGCAAAAGAACAGATTGCACTTTTGGTTTGTACCACCTCTTTACAAATTTGTTAAAGG
AATACTAGGTGCCAAGCCTGTGTGGGCACCATAGATAATGCAGATAAATAGAATACAATCCCAGCAAGCT
CATCTTCTAGCTAGAAACCTCAGAAAGACACAAATAAGAGCAGTAAAAAGGGATTGGCCGGAAAGAATGA
CAGAAAAGAGTAATGGAGAAATCCTAGAGGTGCTGAAGGGAGAAGGAGAATAGATCAAAGATCAGGCAGT
GTTCTTTCTTTTAAATGATTAGCCTTTCATTTCATCCCAACAACTGGACAGCAAGATAAGTAACTGGACT
TCAAACTAGTGAGTGTATTTTTAAGGCCCTGCTTGTTAAAGAAAGGCTTGAACTGGCCTCTCCTCATCAC
TGCTTCTTCCAACAGGCCCTCATCACCTTTTTTCAAGTCAAGATTTCATCCCATACATGCATGACTCAAT
CAGATTTGGAAATGTGGGTAAGAGAAAGATGTCAAAGGAAATGTGAAGTATTCACTTCTCTATTAGTCAC
ACCTTTTACACCATAGACTCCAAAGAGGCGTTAAGCACCTGGTTTTCCTTTGGCTCAGAAAAACCAACCA
CCAAAAACCGCCGTTTTTTACCATTTATATTTAGCCATAAAGAAAGAAAAATAATTAGATAAATCATCCA
CTACATCCAATAATTCTCAGCGCCTTCTCACTCAGTTCAGCCTCTCTGAACAATAGTAAGCACCCTGGAT
ACCAGCCACTTTGGGGGCAACATAGTCAAACTGGCAGAGAAATCAAGTCTATTGAGAAACTGCTTTTTTT
CATGGGTACTAATTCTAGTGTCATAAGGAAATACCTATACTAACTTGCCTATTATGATAGTTATAAACTG
TTATCACAAAACAGTCACTGATATGTTTTATTAGTTAGAATTGGGATATATATATGTGTGTGTGTGTGTG
TGTGTGTGTGTGTGTGTATATATATATATATGGGTGGGTGTTTTGTTGCAGCTGCTGATCTTTTTCTTTG
CAGATGGTACAAACTCTCCCGAGTCAATTTCCTGGGCCTATGTCCCCACCTAGCTGACTGAAGTTATCAA
CAGGGGTCCAGTTTGTGCAGGCTGCTAGCCCTATTGGAAGAGTGGGGATGAGGTGGGAGAAAGCAACCAC
AACGTGTGTGGGCAACCTCAATTGGCACTCATAAAATGTTAGAATGTCAACTCTCTCCCTTGGCCACTAA
ATCTCTCACAGGGTAGTTTTTCTTGCCTAACTCAGGTTTACAAATCAATGTGTATGCCTTGGGGGACCAA
TGGCCTCTTTCCTCCCAAATAAACCACTGGCTTTCTCTTTGTCCCCCTAGGTTATAGCTGAGGAGCCCAC
TCCAATTAGTTTATAGGATTCAAAGCCTCTTTTTAAAAACATCTCTGAGCTTATGAGGAAAGACTTCAAG
TTTCCCAAATCTAGTGGAGGACAGGGCAAGGGAGGAAAGATAGGTACAGGAGTCCACAGGAGGCCAGGTT
TTGGCACCCCTTTGTCAGGAATTCAGCTTCCTTACTAGGGATGAAGAAAATAAGTGTGGGGCTTTGTGTC
TATGCTACCAGAAGGAGGAGAGGATGACACTTCCTCTCTGTTTCCCAGATTAGAGAACAGTGAACCCAAT
GCTGCCTGTTGGCTAGAAAACAAGTGTTAACTTGCTTCTGAGAGACCCTTTTCTCTGTCCCTGCAGATAT
GCCCTGCGTCCAAGCCCAATATAGCCCTTCCCCTCCAGGTTCCAGTTATGCGGCGCAGACATACAGCTCG
GAATACACCACGGAGATCATGAACCCCGACTACACCAAGCTGACCATGGACCTTGGCAGCACTGAGATCA
CGGCTACAGCCACCACGTCCCTGCCCAGCATCAGTACCTTCGTGGAGGGCTACTCGAGCAACTACGAACT
CAAGCCTTCCTGCGTGTACCAAATGCAGCGGCCCTTGATCAAAGTGGAGGAGGGGGGGGCGCCCAGCTAC
CATCACCATCACCACCACCACCACCACCACCACCACCATCACCAGCAGCAGCATCAGCAGCCATCCATTC
CTCCAGCCTCCAGCCCGGAGGACGAGGTGCTGCCCAGCACCTCCATGTACTTCAAGCAGTCCCCACCGTC
CACCCCCACCACGCCGGCCTTCCCCCCGCAGGCGGGGGCGTTATGGGACGAGGCACTGCCCTCGGCGCCC
GGCTGCATCGCACCCGGCCCGCTGCTGGACCCGCCGATGAAGGCGGTCCCCACGGTGGCCGGCGCGCGCT
TCCCGCTCTTCCACTTCAAGCCCTCGCCGCCGCATCCCCCCGCGCCCAGCCCGGCCGGCGGCCACCACCT
CGGCTACGACCCGACGGCCGCTGCCGCGCTCAGCCTGCCGCTGGGAGCCGCAGCCGCCGCGGGCAGCCAG
GCCGCCGCGCTTGAGAGCCACCCGTACGGGCTGCCGCTGGCCAAGAGGGCGGCCCCGCTGGCCTTCCCGC
CTCTCGGCCTCACGCCCTCCCCTACCGCGTCCAGCCTGCTGGGCGAGAGTCCCAGCCTGCCGTCGCCGCC
CAGCAGGAGCTCGTCGTCTGGCGAGGGCACGTGTGCCGTGTGCGGGGACAACGCCGCCTGCCAGCACTAC
GGCGTGCGAACCTGCGAGGGCTGCAAGGGCTTTTTCAAGGTGAGCGCACGCCGCCCCCTCCCCTCCGCAC
CCAGCCCCCTCACTGAAGGGAGCTTCTAAGTGAGTGTAGGAGCATCCTTGTTCTTCCCGGTTTGAGAGCT
GTAATCGGCACATCATGCTTTCCTACAGCCCTTCCTAGCACCTTCACATCCATTTTCTCAGGGGCTTTCT
ACTGAGGTCCATGTGCAGCAGGCCACCCCAGACGGACTCCGGGGGTCCGTGAACCTCCTGGCCTGACACT
GCCCTATGAACATTTTTCTGGAGAGCTTTCAGCAGAGTCTTAAAAGGACCTGTGACTTCGAAAATGCAAA
AACCTCATTGCTTTGTCTGTCCATCCAGGGTGTCTGAGGGGATAGTGTGTGCGTACCAAGCCCTGGACAT
TTGTGTCTCATTTAATCCTTACAGTAAGAGCTGGGGCTCTAGAGTCAGACACATCTAAGATTGAATTCTG
GCCACTCACTAGCTATTTTTAGTAACAGTGTTTTAAAACAGTGTTTTAATTTTCTTTGTTTTGTAGATGA
GGATGTTGAGGTTTGAAGAGGTTAATCAGCTGCCTCAGGACACACCTTCAGAGAAGGGAGATGGAGGCAG
GATGCCAACCCACGTTTGCGTGACCCCAAAGTCCATTCCCTTTTCACAGCTCTGCAGCTCTAGGAGACAG
GGACAACTGTCTTCTTCTATGGAAAGAGCTGCCTCTTTCCATAGAAGATAGAACTTGGGGTCTTGTGTCC
TCTAGGAACCTTTCCCTACCTCTGTATCACATGCGGTGCCAGGTGTAGACATAATTCATTCCCTCATAGG
CCTAACGGAGAAATTTTACAGTGGGAGGGCCATCAGCCTCCCCCATATTTTACCCACGAGGAAGCTGAGG
CCTGGAGCGGTGAAGCCTTATGCAGGCTGTAAGCAGTGATTTAACTGCCCATGAGATGTTTCCAGCTGAT
TTGTCCTGACTCCTGAGTTCAGTGTTATTTCTGCCACTTAGGGAAAAGACCTTGCTCTTTTGCTGTGCTA
CCTTTTGCTGTTGACTCCCAGACTTTATCAGACACTTACTTCTGTGCCATCCTTCTCACTCCTCACCCCA
CTGGCCATTATTTCAGTCATTTACAAACCCACACAAATTATTCCTTATCCTCTAGTGCTGGACAGGGTAG
AAAGAAAGAGAAAGCAGGATGCTGACTGGGTAACTTGGATTTGGGAGGCCAATAGGATATATCTGGGAGT
ACACCAGAATTAGTGAGAGAAGACTCAAATAGAAGACCTTGATCTTCCACCTTCCAATTGTATCATGTTG
GGCATGACACTTCATTCTAAGCCTGTTTCCCTATCAATGAATAAAACTCAGAGAAGAGCTGTGTATTCAA
TGAGATAAAAATATATATGAAAGTGCTTTATAATCTTTAAAGAGCTGTACAGATAGTTCATTATTAAACA
AAAGCTATAAAGTTTTGTAAAAATGCACATTTGTGGAGGGATATACATAGCAACAAGCAACAATTATTTT
TGATCTTCAGATTGTAAATATCACTACCTGAATTCTGCAGAGAAATGAGAAAATGTTTGGGGAAAGACTG
CAATACGTTAAGTGCCCTACAGATATTTGGGATTATGATGTTTTAAATGCAGAATCTTGGTCATTTTATT
TTATTTTTTAGCCACTGAGTTTGGCCATTTTTTGTTTTAACTACTCAAGCTTTATCAGAGCTACTTTCAA
GTAACTGCATTCCTTTAGCAGATTTGATTTTTACAGGATTTCAGTGGAAAATGCTGAAGGACAGTGAAAC
TGGCAGCAGAAAGGGGGAAAGAAAAAAACAAGAATTATGAATAATCTACAAATATAATTATCTCTAAATA
ATGGAGAGTTGACCAAAATACCAGAGTGGTAATACATTCTGAGTCCAGAAATGTCAATTAAGTGTTCCCC
AACAGCTTCTCTTTTCTGCAGCAATCCCTCGAAAACAAATCTATACTTGAAAGGGTGGGGGCAAGATGGG
GGAGAATCACCCCACAGAACTGTTAATTAAGGGAACCGGAGCAGGTTTTACCCAGTGGCTTTCTACCATT
AAATTGAGTTCTTTAAAAATGTGACATGAAGTGCAGTTAATGATAGGAATTCGGTGATTGAACCATAATG
ATCTCTCCCTGTCCTTAAACACAAATGTAGGGAAGGCTTCAGGTTATTGGGAAAATGTATACCTGAAGCA
AAATGCCATCAGCACTCAAATTGGCATAAAGCCTTGAGGATGATGGCAAGAAATAGAGTGTTAGAAATTA
CTGCATGTTCAATGAAAGCTTTATTCAGGAAACACACGTTTCCTACCCTTACAGACTGCTAAGAATTTAA
ATGCTGGAACCTCAGAAGTATGCCTGTGTTTCAGAATTTTGCATTTTAAACTAAAATCACCTACTATGGT
GGGAGAAACATGTCTTAGACTTGACATAGGTTGGCTTTGTTATATTAAGTTTTATATAAGCAATCTAGAA
ACTATCCCCTAAAATGAAATTAAAAGGTAACAGGGTAAAACTGAAACATGGGGGTTGAGAATGACTGGAA
GTAAATGTTAAAAAGCAAATCTAGATGTTTGAACACAAAATAGATCATAAAGCCCAAGTTACCTGGATTT
ATTTTCTTTTTTTATTAATGCAGCCCAGGCTTCCCAAATAAATTTCAACTTTCTGCTCTATTTGGAGTTG
TATAGATCCAGGTAATTTCTGAACAGTTTCACCCTAAGTGGTAAAAATACGTTCATTTGCACAAATGTTT
TCAATTTAAAAAGCAAGCATTTGTTTAAATTTGGAGGACGAAATATGTTTATTTCTCCATGTGTCATTTC
CAAATTATATGGATTCTGAAAAATACAACATCATTTTTCTATCCAACCTTAAGTTTTATGATTTTTTTTA
CAATATGGTTACTGTGTCTATGTCAATAGACATTTATGAAATGACCTATTAATAGGAATTTTCACTGAAA
ATATATGGCTGTCATATATCTTTCTTTGTCTCATTTTTCGTTTGTACCTTGGGGTTTTTTGTTTTCTTCC
TTGGATATAATAATGATTTGTAATAACTACTTTTAAGATTTTGCTTTGAAAAATCAGGAATGGACTGTTT
ATCATATATGGTCTTTAAAGATAAAATAAGCTATACAGGCTGATTAAGTTAGTTTTTTTGCATTAAAACA
TTATTTCCTATATGTTTTAATTAGAAAAATCCTGATTTTGGTTTTTATGATTTCATCTCTGTTGGGTAAC
AAAGCCAGTTACTGTGATATTTGACTTACAATAGTGATAGCAAACAAAGTACATCCCAGATAATTTTGAT
TTTATTTTAATAAGGTCAGCAATAATCAGGGGAGTGGAACAAGTAGAAAATTGTTCCCAAAGGGATATTA
AAAGTGGAGATAATCTAATTCTCCAAGGTATGATATGTTGACCTGGTAATTTCAGATATGATTTTATCAT
TCAGAGTTGGGTGTCTCCTGAGACTTGCTTTAGAACAATCCGAGATGATTTTCATTGTCAGCAGCTAACA
CTAATAAAATTGTTTTTCCTGCAGGCTAGCGTTTTAGGAAATTAAATTTATCGAATTATACGAATTGCAC
TGTCGCTTTTCACATTGTAATTAAAATACATCTTGTCAGGTCCTACCTCTTTCCAGAAACTATCAGTTGA
CTATCTTGTATTATATTTTCTCAGAGAACAGTGCAGAAAAATGCAAAATATGTTTGCCTGGCAAATAAAA
ACTGCCCAGTAGACAAGAGACGTCGAAACCGATGTCAGTACTGTCGATTTCAGAAGTGTCTCAGTGTTGG
AATGGTAAAAGAAGGTATGGATATAATGCTTTTTACCCAGTTTGCTTATACATATTATCAGAAATCAGTG
AAAAAAAGCTAATATTTACATTGGGATGGTGAATTTTTCTAGTTCCTTTTTCCTTTCAACATTTTATTTT
TATCATATATATCTACATTTTAAAATAACTGAGGACTATTTATTCTAATTTCTCAAAATTAACAGAGGAC
TTGTATACATACATCTTTGTTCTATTTAAGAAGGAGACTTTTAGGGCTAATTCATTACCAAATGTAAATA
TCGGTGGCTTTTATGCTGCTTGTTTCAACTCTCATGAAATCATTGTGTGAATATTATTCCAAACTTATTA
CCACTTTTAAATGGTTGGTCTCATTAATGATTGCTCAACTGTAATGTGTTTTTATTCCTTTTTGCGATTT
ATGGTCGTTCATTCCATAATCTTTGAAGATTGTTTTCTTTGTCTCTTTTGGCATCAGTTGTCCGTACAGA
TAGTCTGAAAGGGAGGAGAGGTCGTCTGCCTTCCAAACCAAAGAGCCCATTACAACAGGAACCTTCTCAG
CCCTCTCCACCTTCTCCTCCAATCTGCATGATGAATGCCCTTGTCCGAGCTTTAACAGACTCAACACCCA
GAGATCTTGATTATTCCAGAGTAAGTTTTATGATTTCCTGCTTTCAAATGAATGATCAGGGTCTCTATTT
ATGGCTACTAGTAATAAGAGTTGATTGAATGATTTTGTGTCTGGCACCATGTTAGACAGTTTTCATACTT
TTTCTATATTTCTCGCTTCATTTAGCAATTCAGTGCATCCATTGCAGCAAATAATTTTTGCCTTATTGAA
TCTCTAAATGCCTTAACAAGTGACCCTGACAGTGCTGCACCTGTCATACACATTGTTGCAGGATTCCTGG
TGGTTGTGCCAATGAAAATCTGCACAGACAAACTACAATTTGTAGATTTATCTCGTGATCTAGACAAAGT
GACTACTGTTTTTTTTCATATTGTGTTCAAACCATCTGGGTGAGCCTCAAGTTATTACTAAGCAGTTTAT
CCAATTGCATCAGCATTGATTGACCTGCTGCTTATTCAGAGGGATTAGACCATTGGTGGAGAGAAGCGAG
CTCTGGCTGTGACATCAGTTGTCAGGAGAACTGGGATGGAACGCCACCTCTGCTACTTCCTGCCTGTGTC
ACCTGGTTCCTGCTTTTGGCTCTCTCCTGCCTACAGTGGAAATGGCTACAATATTTAACCTCAGAGGGGT
GTTCTGAACACCAGTCACACAAGAATGATGGTTGTGAAAATGTTTTTAAGACCATAAATGACGATTGGTG
TTGTTATAGTTAAGTCAACCAAAACATATCAACATTTTTATTTCATATGGGATATAGTAAAATCCCATCA
GTCTGAACCCTGCAAATTCCTCAAAATTTGGATAACACAGAGAAGAACCATGAGAAAGGATTTCCTGAGC
AAATGAATAAACAGCTCTGTAAGGCAATTTCTTTAAAGGACTTAGAAAACTAAACTTCTAGCAAAACAGG
GTCTCAATGAAGAGGGAAGAGAAGGAAAAAAAATCACAATTATTGAGCAGTTTCTATATGCTAGACATTG
TGGTACATTATCTCATTTAATCCTTTAAACCACCATGAATTGTAGGAATCATTATCCCTCCTACTTGAAG
GAAAACAAAAACAAAAACAAAAACAAAAAACCTTAGAAACACAGCTTGCACTCAGATAAAGAAATACCAG
TCCAGAGGCCCCCAGTCGGTTGATCTGTCTTGGTGGCTACTACCAAACTGCCCTCCCCAGGTCTAATCAG
TGTTTCAAGCTTCTGGCTGAATGGCTGGCAAACAGTGTGAGTGTGTCCCTGCTATGTGCCCTTCTCCTTA
GGTTGCAGTGTTGATAGAAATGATTAGTGTACCTGCTGTGATCCTCTTTCCCTCTTCCCCACCCTCATTA
TAGGCTGCGAAGCCTCCTCTCTGCACCTGATAACAAAACGTCATATGAGAAGCATGGTAGATCCTTAGCA
TCAAAGGTTGAGGACTCTTATTCTGATTATAAGTAGTGGCTCTTGACTACAATCAAGTCTCAAATAATAG
TGTAAGAGAATAAAGCAGAATAATAAGACTAAGTTAACAGTTTAGGCTTCTTTGGAATCATGCGGGCCTA
GATGAAAATCCCAACACTGTCCTTTACTAGCTAAGTGACCTTGAGCAACTGATTACACCCTTTGATGCCT
CAGTTTTCTCCTCTGTGTTGTGGGGTAATAGTAATATCTACTTCCTGGGGTTGTTCGTGAAGATTAATTA
ACAATTATACTTGTCAAAGCTTTAGCACAGTGCCCTGTATGTTATTTCCTTGGCCAAACTTTCTTACTCT
GCCATTTGTTCAATGTCCTAATGAGCATGAACACTACATTAGGTATCATGCAGAACACTCTAAAGATAAG
TATTATGATCTCTATTTCACAGATAAGGAAATTTAAACTGGGAGAGGCTAAAGGGCTGACTTGCCCAAGG
TCACTGAACTAATATGCAGCAGAGACAGAATTAGGAGCCAAGTATATTAAGAGCCAAGTGTATTGAACCT
AAAATCTGGGCTCCTAAATACCAAGCTTCACTGCCTCTCTGGTTCCAGTGAGAGTGGGTGCTAGAAAAGT
ATTCAGGAATGAAGAGTTCTCCTCCCAGAAGCACCCTGGCCTCTCCAAAGGCAGTTCATCTGAATTAGTG
AAGCTTCTTAGCACTGCCTAGGCTAAGGCTATAGCACACAATCTGACATTGCTCCAGGGCATTTGAAAGC
ACAATAGATGGTCAGTACAGATAATTTAGGCCTCAAAAGTAATTTTTTGGCCCCAAATTATCCAATTATC
AGTCTACATGGCCTAATTAGTCATTCAATGGCAAACCTTTCCCAGCACTTTGAGTTGTCCCAGCTGGGAA
CTGTCCCCACACTGAAAAATCAATCTAGTGTGTTTGCTTGCCTCTGCACCATTCAGTCTGTTAGTCTAGA
GTCATCATCCTCATCAACAAACATTTATCCAATTTAATTGTTAGCTTTGTCCAAAATAGCAGATTGACCA
TGCCAGAGCCTGGGTCACATTGGTACTACCTTTGGGGCCTGGCTCAATTTTGCAAAGCATATTTATTTTA
ATTGCATTATTTTGGAATCAGGTTGAACCTCATAACTTGGATTCCCCTGCTTTTAGATGGCATTACACAA
TTATCAAAAATTGGACCCAAATCAACTAAATTAAAAGACTTTTTAAAAATACAGTGAGTTTTCATCAAAA
ACTTGGATGAAAGCATAAAAGCATGCTGATCACATTTGCAAATGACAGAAAGCTGGGAGGGATCGCTAAT
ATGATGGATGTCAGTATCAACATTCAGAGTGATCTCGACAGGTTGGAACAGGAAGGGCTGAAACCATCAC
AAAGAAAGTTAACAAAAATAAATCTGAAGCTCAGCATTTAGAATTTAAACAAATGGGAGAGCCTTGGCAA
AGTGGTTCATGTTAAGAAGATCTGGGGAGTTTATTCCATATACACTCAAAATGAACCAACAAAGAACGTG
TGTGGTAGAAGGGAGGGATGCCACATTACCTTCAGCTGCAGTAGTAGGAGTATGGGCAGACCCAAGTTGA
TGATGGTTGCATGGACTCTGTCCTGTCAGACTGTTCTTCGTGAATTGAGTCCAGTTCTAAGCTGGGATTT
ACTGTTTCTGGGAACTTGTAATGGATGATTGGGGGAAACAGAAATATGTAGGCAAGAAGGGAAAAGATTT
GGTGAAATTGGGTCACTCTTTTGCCCTATGGCAAAAAGAACTATTCTCCCAAAGACAGATCTAGAGAGAG
GAGGGGGGCCTCTTCCCGCTCACTTGTCTGATAACAAGTGTTGGCCATACTAGCTGCTGTGGAAGTGTGA
GTTCATCAACACAGGGAGTAATTAAGGTAGTTTTAGTTTTAGCTTTTTGGGGAACCCCCATACTGTTTTC
CATAATGGCTATACTAATTTACATTCCCATCAGCAGTATATGAGTGCTCCCCTTTCTCTACATTCTCACC
AGCATTTATTTTTTGTCTTTTTAATAATAATCATTCTAACTGGGGTGAGATGATATGTCCTTGTGGTTTG
GATTTGCATCTTCCTGTTGATTTGTGATGTTGAGCATTTTTAAACATACCTGTTGGCTATTTCTATGTCT
TGTTTTGAAAAATGTCTATTTAGGTTTTTTATCTATTTTTAAATAGATTTTTTTTCTGCTATTGAGTTGA
ATTCCTTATATATTCTGGTTATTAATCCCTTGTCGGATGGATAGTTTGCAAAATTTTCTCCCATTCTGTA
GTATTTCACTTTTTAAATTGTTTCCTTTGCTGACCATCACATATATTCTTACATAATTTCACTTAAGACT
CCAGCTCTGTGTGGTATTATTTATGGTTTGAGTAATTTCCTTAATAATGTCATTCACTGAAATTCAGTTT
TCATAATACCTTCCTTCTTAAAGTTTTACATATACAAATTCATAAAACTGGAGTGACCTTGGGATCATCT
AATCCAAGTCCCATATTTTCAGAAGAGTAAATTAAGGTTTAAAGGGGTTAACTGGCTTGCCTAATGTCGG
TGACAGAGTAGAGGACAAAATCCAGATCTCATCACTCAATAGGTTTCTATTTCATCATACTGTCACTTCT
ATTTAATGACAATTTATTATATTTTTGAACATATAAACTTTATTAAATATTTTTAAATTACAATAATAGT
AATATATATCCATTGGGGTTTTTTTTTAAAGGCAACCATTTACAAGTGGATAAAATGAAAAATGAAAGTC
TCTACTTCATTTATAGCTGCCCGATCCCACTCCTTAGCCGTGTTTTAGTGTGTATCCATAATCTATTACA
TTATAAATAAGAATTTGCCCTGTCAAGTTTGAGAGTTGATTTGAGCCCTACAATTTATTAAAGATGCTAG
CTTCCCCCAGTGAGTCAGGTCAATAGAGAAAGGAGGAAATGTGTTGCCAGCCCAGTATGTTTTAATCTTC
ATGTACTAGCATGTGTTCCTTTAAGGTAGTAAGTGAGCAGTTTTCTCCATTTGTGGGAATGGGAGGTGGA
GGGGTTTTGTTCCAAGTCACAGAGGAAATGAATGTTTTAGAGGCCAGAAAGAGCTCCTGAGTTCTGTGAA
GCATTCCTGGCTCTGGGACCTTCAGTTCTCCTTTGGGCCAAACTTCTCTCCCTCAAAAGTGAGCTCAGAT
CATTGTACATCAGTGCAGTCAAAACATGTCACGTTTGATTTGTCTCTCCTCAAAATGTTTCTGTTCTCTG
CACCACAGTTGCTTTTAATTTAGCAATACAAGTATGTATAGATACTGGGATTAGCTTCAGAGCCTGCATA
ATTACTAATGAGAAAACATACTTAGTGTCCTCATTAGTTGATCATTTCAAGTATTTTTCAGCATACTAGG
ATGTTTTCTGGTAATCAGTGCCACTTAACGGAGTAACAGACTTGCAAATACGAGAGGTGCTGCTCAGATA
AAACCAGGCACCAGCAAATGGCATTTAAATGTGTTCACAAGATAGCCTATGAAACAATGTGGCTTAAGAC
ACGTTAGAACTACAAGGTAAACAGAATGAAGCTGCAAAAAGAGTGGTTTCATGAAGCATCTCAGAAGACC
CTCTCAAATTGTTACCAATTCACCAGATGATTTCACGCCAGTTCCTGCCCCTCTGTGGGCCAGTTTCCTG
ATCCAAATGGTAAAGAGATTAAGCTAAATTCATTCCCATTATCTCTCCAGGCTTCTCATTCCCTGATATT
CTGACCATAATATTAATAACTGCTATAGTTCCACTATGAGTACAGGTCAGCATTTCCTAAAACATGGTGC
CCAGACTGCTCCTACGAGAAAGAGGTGGGGTGTTTGCTTTAAAATGCAGATTCCCAGGCCATACCCACTC
AGACATACTGAATCAGAACTGGGAAGAAGGAGAGGCAGGCAAAAATCTGTACTTTAAAAAGTGCCATTGG
ATGATTCTTTGGCACACTAAGGTTTGAGAACCATCGATATAGTTTATAATAACAACTCAATTTTACCTTG
AATTTTCCAGCTTTTCCTGGGGTTGAGAAGGGATGAGCAATAGAGATATAAATTTTCCTGAAAGCAATCA
ATTCATTTAACAAATACTTACTGAATGGCTGCTAGGTAGTAGGCACTGTTCCAGGGCAATGGACACGTTG
CTGAACAAGACAAAGCCCTTATCCACATGAACCTTACATACCTGTAAAGGAGAAAAAGAGTAAACAAATA
TACAATTGCAGTGATGTCATTGGTGGGAGGAGAGGAATTTTTTGCTTTTTGCTTTTTGGAGTGGGGGCAT
AGAGTTAGATCAGAAAAGAAAAAATTGGGGGGAAAATATATTCATTGCCAATTTTTAAAATGTCACTTTT
TAAAGTGTAAGAACCTAAGAATATGTATACATAGTTTGACTTATACAATGATCACATCTAAAATTTTTAG
AGCTATAGTTGAGAAAAGTAACATTTTAAGGGGAGAAAAACGTGTCCTTAGCGTAGTCTACATATTTAGC
CAGGGCTGAAAGTGAGATAGAGTAAATATTAGATTCCACTCTGCTATTAAAGCCTCACATCACTAATTTT
TGAGGGGTGGTGTTTTCCATGGGTCTCACTTAATTTCCACACAAATATCTCATTTGGGGCCTGGGCTATT
GCTGAAGTCTGACTTGTATAGCTGCGTTACTGCCATATGAAACACACAGACCCATTTTAGTTTACATAAT
ATCCATTGCTGTTGTTTGCAGCTCTAGATTCCCATTCTAGGTGCTTTAGAGAAACCTTCCTTAGGCATTG
GCTGTCAGTAAATGTAATACTGTGTCTTTGACTAGTGAGAAAGCCAGAGTTCTGACAGATCAATAACCCC
TATAGGGTGGAAAAAAATTAGTATAAACAGGAAAAAAGTTCATTTAAAAAAATCTTTTTGCATTTGACCT
ATGTTCGATTGGCATGATCAGTAAGCAAATATTTCTAGATTTTCTTTGTCAAACCCCAAACCTACTTAGC
CCAGAGACAGAGCAATCAATGTAGGGCAGCAGAGACACAGAGCTGGGAGTCCAGTCCTTCCAACTCTAGG
ACCAGTATTCATTGGGTGAGGTTTTCCTAAACTGGTAGGCCAGGCAGAGAAAAAATCTAAAACGTTTTGT
TCCGTTCCTTTACATCTTATGTCCAATAGAGGAGATTTTTCTTTTCCTCCAGCATTGGATGCTGACCCTC
CAGTCACCCCCAAGTTACTGGTGGCTCAGACTGAATTCACTTTGGCTCCAAAATTCTGAGACTTGGACCA
AAACCACTGCAGGTGAAGCCCAGAGGATCTGGCTGGAGCCTGGCAGGCTGGGCCGGCTGGCTTTCCTTCT
TGCTGGGCTCCATCAGAGAAAAGTACACACACAGGGTGGGCAGGGACTTCACTTCCCTGTGTGCAGAAGG
CATGAAATGTGAGCCCAGCAGGGGCAGAAGCCTGCAGAGGACCCTGGGTGAAAGCTACACACTTTGATGG
ATTCTGAACAAATATTGGAAGCAGAGAGATTGTTGAGTTGTGAGCCATGGATTCAGGGGAGTCAGTGCAG
GAGGTAGCTGTCAGATCCATTCTCAGGGGAAACTATTCATTCTTTAGTCTTTTTCTCTCTCCCACTATTT
TAAAACAAAATAATGCTGAATCAGTGTCAAGTTCCAGGCAATAACATCATACCTGGTGTGATTTAGCAAT
ATTTAGAATCATTTAATGCAAGAGCCAGAAGTAATCTTAGGGATCAGGTAGTCCACTTTATTCCTGTTCC
AGAGACTGAAACTGACTCAGAGAGGTTAAATGCCTTGTCTAGAACTGCACAGCAAGTCAGTGCAGAGCCT
GGATGAGGACCCCATGACCTGCTGCTTGGTCCAACACACTTTCCTTTACTCCCACTCATTTGGGAGTTTC
ACAAGTAGCTCCCTCAGCTTTTGAAAGGGAGGATCTGCCCTGAATTTCATTCTGCTCTTGGAGAGCCTGT
GGAATTATTAAATAAATTCATAAATAAGGAGAAAATAAAAAAGGGGAAAGTTAATTCCCTTTTCCCCACT
CTCTTCCAGTTAAGAGTCTGTGTGCTGGTGGGGCGCGGTGGCTGATGCCTGTAATTCCAGCACTTTGGGA
GGCCAAGGCAGGTGGATCACAAGGTCAGGAGTTAGAGACCATCCTGTCCAACATGGTAAAACCCTGTCTC
TACTAAAAATACAAAAATTAGCTGGGCATGTTGGTGTGCGCCTGTAGTCCCAGCTACTCGGGAGGCTGAG
GCAGGAGACTCGCTTGAACCCGGGAGGCAGAGGTTGCAGTGAGCCAAAATCATGCCACTGTACTCCAGCC
TGGCAACAGAGCAAGACTCTGTCTCCGAAAAAAAAAAAAAAAAGGAGGAGGTAGTGGCAAGTGTCTATGA
CATAGGATCGTGCCGCCATGCTTCCTTCTCCTGGACTTTGCTCCCATGGTCTATTACAACACAGAATATT
CAGGACTTCATAGAATCCCAGGCATTGTGTAGGATTGTTGGGAACAGAGAAGCCAGGGCTCTGAACTGGA
CCAGATGTTATAGGATCTTAGAACATCAGGGCTGAAAATCAAAAACTTTCTACTTCAACCCATTCCATTT
CATAAATATGGAAGCTGAGGGATGGTGATTATTTCAGACATCATAACTGCTATCAATATCATCACTGTCA
TTTGTTAATCATGTACTATATATCAGGCCCTTTACATATGTTATCACATTTAATCATCACAGCAAAATTT
TGAGGAAGGCCAGGGATGGTACACATGGTAATCCCAGCATAGATTGTAGGGAGGTCCAGCCAGGTGGATC
GTTTAAGCCCAGGAGTTCAAGACCAGTGTAGGCAATATGGGAGTTCAAGACCTGCCTAGGCAATATAGGA
GTTCAAGACCAGCCTAGGCAATATAGAGACACTGCCTCTACAAAAAATAGAAAAAATTAGATGGGCGTGG
TGGTTCCTGCCTGTGGTTCCAGCTACTCAAGAGGCTGAGGTGGGAGGATCGCCTCAGCTGGGGAAGTCGA
GGCTGTAGTGAGCCCTGATCACACCATTGCACTCCAGCCTGAGGAACAGAGCGAGACCCTGTCTCAAAAA
AAAATTTTTTTTGAGGAAGGTATCCCCATTTTACAGAGTAGAAAGCTGAGGTTCATAAAAGCAAAATGAT
CCTACATTATTTCCCATATTAGGGGCCAGAACAAAATTGAGATTAAAGTAGCTGGACTCTCTGATTTTTT
TTTTTTTTTTTTTAGTTATTTAATACTGCTTCTTGGGAAAACACAAAGAAAAAAAGACCCAATTAAATAA
GCGTCTCATTTCCTCTCACCCAGGTAGCTCCACAGTAAGCACCTAGTTGCTTATTAGCTGTTTCTTCTCC
AACCAGGTTAGGATATTACAGCAAACCTAAGCACTCTTCTGACTATTCTTTCATTGAAAAAATATTCAAC
AGTTATTTATTGAGGTGTAATTATGTGCCAGTCACTGTACTGTTGACAGAAACATAGGACCAATGAATAC
ATACCTGGAGAGCTCCAGTGAGAGTAAAGCAAAATGCTTTTTAAAGATTTCATAGGCCAGACGCGGTGGC
TCACGCCTGTAATCCCACCACTTCAGGAGGCCAAGGCAGGTGGATCACTTGAGGTCAGGAGTTTGAGACC
AGCTTGGCCAACATGGTGAAACCCTGTCTCTACTAAAAATACAAAAGTTAGCCAGGCATGGTAGCACACA
CCTGTAGTTCCAGCTACTCGGGAGGCTGAGGCACGGAATCGCTTGAACCCAGGAGGTGGAGGTTTCAGTG
AGCCAAGATCGTGCCACTGCACCCCAGCCTGGGCGACAGAGCAAGACACCATCTCAAAAAAAAAAAAGAT
CCCACCAGCTAGTAACATCAGACATACTTAATAGCTAGGAGGGGTAAAACTTGAAATCACAGGGTCAGCA
TAAGTCCTGGCTGTACCACTATTGGTTGTGTAACTTGAGAAAATCTCTTAGCTTCTCTGAGCCTCAGTTT
TTTCACCTGTAAAATAAAGATTAAAATCCCTACCCATGAGGATCAGTTCAGAAAAGGAATGCAAAATGAT
GCACAGCCATGTTAATGTGAAGGAAATGATGTGCCTATTTACGTGTTTCTGTGTATCTCTTAAATCTCTG
TGTTGTTGGCTGTCCCTTGATTAGTATCTTGCTGTGTCAGTCTTCCTGCTTGCAGCAGAAAAGTCTTTAC
TCCGGTGATTCAAGAAAAACTCTGGTTCCACCAGAATTTCTGAAAGCTGGGTGAAAACTTTATTCCTACT
TATATCGTTCTGCTCTTCGGACTCTCTAGATCCATGCTGTCCAATAGACCTCCCCTCAGTGATGGAAATG
TTCTATCCCACATTTGGCCATTGAACACTTGAAATATGGCTAACATGACTGAGGAACTGAATTTTTAATT
TCATTTAGTTTTTATTAACTTAAATAGCCACAAGTCCTGAGTCTGATGGCACACAGCTGCCTGGGTTCTG
CCAAAGAGACCTCTCTTTCCACCACTTCCTGGTTTGTTTTATCTCACAGAAATTCATCAACAGCCAGCAA
AAATCATTCTTTCTCTTGTTGATTATTTCTGCGATTCTCAAACTTCATTGTGTATGAGAATCACCTGGAG
ATCTTGATAAGACAGTTTGGTGGGCCAGGCACGGTGTCTTATGCCTGTAATCCCAGTACTTCGGGAGGCT
GAGACAGAAGAATTGCTTGAGTCTAGGAAATCAAGACCAACCTGAGCAACATAGCGATACCCACCCCCAA
CCCACGCCCCCATTTCTACAAAAAAAATTTTTTTTTGATGTTTGCTGTGTCTACCCCCAGAGTTTCTAAT
TCAGTAGGTCTGTGGTAGGGCCTGAGATTGTCATTTTTTTTTTTTTGGAGACAGAGTGCCACTCTGGCAC
CCATGCTGGAGTGCAGTGGCACGATCTTGGCTCACTGCAACCTCCACCTTGCAGGTTCAAGCGATTCTCG
TGCCTCAGCCTCCCGAGTAGCTGGGATTACAGGCACATGCCACCACACCCAGCTAATTTTTATATTTTTA
GTGGAGATGGGGTTTCACCATGTTGGCCAGGCTGGTCTCGAACTCCTGACCTCAGGTGATTCACCACCAC
CCCCCCACCCCCCAGCCTCCCAAAGTGGTGGAATTACAGGCATGAGCCACCGCCCAGGCCGAAATTATCA
CTTCTAACACATTCCTGGGTGTTGCTGATGCTGCCAGTCTGGAGACCACACTTTGAGAACCACTGGGTTA
ATTTAGCATCTCATGGGGAGACAGCTGTGCTATAGTGAAATGAGTAGACCCTTGAGATCTACTTGGACAC
AAACTCCTGGGGCAGTAGTTCTCAATTGGGCTACTCCACTTTGGAGTCACCTGGGGAGCTTTTAAAATTC
TTGACCCCTGGGCTCCACCCCAGACCAATTAAATGAGAATCTCTAGGCGCAGGACCCAGACAAGAGTTGT
TTCTTCAAGCTCCCCAGGTGATTGCACTGCACAGTCAAGGGTGAAAACCACTTTGCCAATTCGGCTTAAG
CAAATTTCCTTGTCTATTAAGTGATAACGATAAAATTTGCTGTATTTGTATCACAGGGCCATTGTGAGAA
TCACATGAGACTGTGAATGTGAAGGGGTTTTATAAACTGTAACACCTAAAAGTAAGAATTTAGGCATTGA
TGGTCTTGGAAGGGCCTGTTTGCAAGTGATGCCATCATCCCCACTGAAGCAGGATAATGCTGCTCTCTCT
GGTTTGCATTCTAGTACTGTCCCACTGACCAGGCTGCTGCAGGCACAGATGCTGAGCATGTGCAACAATT
CTACAACCTCCTGACAGCCTCCATTGATGTATCCAGAAGCTGGGCAGAAAAGATTCCGGGATTTACTGAT
CTCCCCAAAGAAGATCAGACATTACTTATTGAATCAGCCTTTTTGGAGCTGTTTGTCCTCAGACTTTCCA
TCAGGTAATTACTACTATTTTATCTTCAGTCTACGTCCTTTGAAGAAGCCTGAAACCTTCTGTGTTTGTA
ACTGAATCATTGGTGAAACGTTTTCTCAAGAAGCAAATGTGAAATATGATCAGGCACTAAAAAGACTGAG
CAGCGAGTCACGGAGGGAGCACTAGGCTTGCCACCAGGACCCGTGGGTTCTAGTCCCAGTTCTAATGTGG
ACTTGCTGTCACACCCTGAGCAGGTCACTTGCCTGACGGGAAGTTCAATTCCATCCTCCTTAAAAGGAAG
AGGTTGGATTTGATGATCTCTTGAGGTCCCTTGCAGCTCCAACATTCTATGATTCTCGAAAGATTCTCAT
AGTGCATAAGCTATTTCCTGGGAGCAAGTGTTTGCTTTGGAACATTGTATTTACCTCGTGGGTTTCTGTT
TAACAAGTTACTATTCTAGCACACCTGATGGGGCCCTAAAGAGTTCCCACACCTTCTCAACCCCCAGTTT
TATTCCAAGAGAACACTCTTAGATAGAAAGATTTAGTCCATCCTATCATACTCAGTGACGCCAAACACAA
AAAGGAAAAGTGATTTTTCAAAAACATAGAACCTTGGATCTAAGCTCCTCTAGTAAGTTCCACATGTAGA
TTTTTTAAAGTTTATTTTATTATTGTTTTTATTCACAAATAGCCTGTTTAACTCCATCTTCCTTGCTTCC
TTTCAGTTATATATTTTTAGGAGATGATACCAACTTTTTACCAAATATTATCAGAAAAATCCATCTTTCC
CCTCTTTCTCTTTCCTGCCATCACCTCCCCAATTTCATATCACTACCAGAAACTCCTGTTTAATTTTTAA
AGTGCAGATTGCCAGGCCTCATTGCCATCTATTTTGGTTTGGTAGACTGTGGAAGTCCAGAAATCTGCAT
TTTTAACATATGCTTTAATGATTCTGATGTCATCAGGGTCCATGTTTTATACTCTGAGACTAAATCATTC
TGTCCTGTGTATGCCTTCTGTGGAGGGATCAGTAGGAGAGATGGGGTGGAGTTACAACTAGAGTGCCAGT
AATAGCTTAATTTGTAGCCAATCTAGTGTACACCAGAGCTGAGCCAGCAATGTGACTGTCATCTATTAGG
CATGCAACAAAGGATTAAGTAGACTGTAAGCTCCATGCAGGCAGGTGCCATATATGTCCTATTCACCATG
TTTTCTCCCAGTGCCTGGCTCCAGGAATAACACATACTTCATGCTCAGTAGACACTTGGCTGAGTGAAGG
AATGAATGAAGCTGTTCATCCAAGAAGCACAAAGAGATATTTGACTTGTGAATACAACAGGCCAAGATAG
ATACATACGTGGCAAGAGATTTGAAAGTTAAAGGAAATCTAAGATTAAAGGAAGAACCACATATAATTCA
GCCCCTTACTTGTTATACCCAATATTTGTCGAACCATCCTGTGTACTGGGCACCATGCATGGGCTTTCAA
ATCCTTTCTTTTATTTAATTCCCTCAACAATCTTAGGAGGTAGCAATTAGAATTTTTGTTTTATAAATAA
GGAAAAAGAAGTCCAGAGAGATTTAAGGAGCACCCAAAGCCACACCCCAAGTCATCATGTCCACCTGGAA
GTTCCAGGGCACCTATCATGAAAGAAGAATGAGATGATGATGGACATAATATAAACTATCAGGACAGATT
TAGAAATAGCCCTCTGCAGCCCCAATGTTAAAAGCAGAGAAGGTAATACAAAATAAGGACTTGTTTTATG
TTAGGCCACCCTTTAATTTGTGATAGACTTCTGGCACTTACAGGGACTTGATTTTTGTTTTGTTTTGTTT
TGAGACAGAGTCCCACTCTGTCACCCAGGCTGGAGTGCAGTGGCACGATCTTGGCTCACTGCAACCTCCG
CCTCCCAGGTTCAAGTGATCCTCCTGCCTCAAGCTCCTGAGTAGCTGGGACTACAGGTGCACACCACCAA
GCCTGGCTAATTTTTGTATTTTTAGTAGAGATTGGGTTTCACTATGTTGCCCAAGCTGGTCTCAAACTCC
TGACCTCAGATGATCCACCCGCCTTGGCCTCCCAAAGTGCTGGGCTTGCAGGCGTGAGGCATCGCACCTG
GCCAGGGACTTGGTATTTTTAAAGTGTGCACAATGGAATTGTAGTGTCTAGCCTCAGGCCTTGGCAGTTT
TTAACATTTAAGAAAATCAAGGACAAGCCCTGTCTCAGGATCCCTGGAGGGAACAGTCTCATGTTCATTG
CTGAGAGAGGGGTGCACTGGGCTTCATCTCTGGAACCCATGAGTGCCTGCTCCCCCATTTCCTGTCCCAA
GGAAAGCCAGTAATTGATTCTAGTAATAAGATAGCAGGTGCCACAGCCCATATCTTCCCCTGGGCTGATG
TTCACCAAAGACATTAGCATAGACAAGTGGTGCTAGTTGTTTATCCCAGAGAACATAGTCTTATTCATTG
TGGGTATCTGGCATGCTTCATAGCACCACACTGGGGTCCCAATGCTGCCCCATGGCTGTCTGTGTGCCTG
TGTGTCATAATGTTATCATGTTGGACAGATTGAACAGACCTGTTTAATGTTTGTCTTCCTCTCACCTCCC
AGGTCAAACACTGCTGAAGATAAGTTTGTGTTCTGCAATGGACTTGTCCTGCATCGACTTCAGTGCCTTC
GTGGATTTGGGGAGTGGCTCGACTCTATTAAAGACTTTTCCTTAAATTTGCAGAGCCTGAACCTTGATAT
CCAAGCCTTAGCCTGCCTGTCAGCACTGAGCATGATCACAGGTAAGCACCACCTTGCCAAAACCGCATCC
TCATTTCTCTCCTTCCCTTTGTTATGGTGGAATCTGGCCTTGACCACTACACACACCAGAAAAGTGGGAA
AATGACTACCATTTTTCTGAAATCTGTTAAGTTTAACCTAGTTTAAGCAAAGAAACATGCATCTATAATT
TGTTGTCATAAACTTCTAGTAAAGAGGATCCATAGTCTATTGAATTATCATTGTCAGTAAGCCCAATATA
CGAAAATCTAACTTCCTACTAGAGATCTGTGGACAGTGGGACTAGCTGCTTCAGGGTTTGTTTGTTTTGA
GACAGGGTCTTGCTCTGTCACACAGGCTAGAGAGCAGTGGTGTGATCATGGCCTAGTGTAGCCTCGACCA
CCTGGACTCAAGTGATCCTCCCACTTCAGCCTCCTGAGTAGCTAGGACTATAGGCACATGCCACCACACA
TTTGTCCTGTTTTGTAGCCCTGTTTCACCCTGTTGCCCAGACTGGTTCAGTGTTCTTGTTACAAAGCCTT
TCCTAGGGAGATTACCTAACCCAGCCCATTCCTGTGGAGCAGAGGTGATGAGGTTTCCCCTGTCCCCAGT
TCTTGGCTAGCGACCTAAAGTAAAACCAAAACACTATAGACATATCAAATACAACTTGATTTAATAACTT
CGATTGGCAAAGGACTGAGAAAAAGCAAAAATGGTTCAACTCAGCAGTTTTTTATTTTTATTTTTAAAAT
TTTATTATCATTATTTTTGAGATGGAGTCTCACTCTGTCGCCCAGGCTGGAGTGCAATGATGTGATCTTG
GTTTACTGCAACCTCTGCCTCCTGGGTCCAAGCAATTCTCCTGTCTCACCCTCCTGAGTAGCTGGGATTA
CAGGTACCTGCCACCATGCCCGGCTAATTTTTGTATCTTTAGTAGAGATGGGATTTCATCATGCTGGCCA
GGCTGGTCTCGAACTCCTGACCTCAATTGATCTACCCGCCTTGGCACCCCAAGTGCTGGGATTCCTGGCG
TGAGCCACCACACCCAGCCAACTCAGCAGTTTTTTAACCACAAACATAAAAGATATTTGATTTATGCCTG
TCTCCCTCTATAGATCTCTCTCTTTCTATCTTTTGGGAGATAAAGGATCCCAAGGTGGGGTGGGATGGAG
AAAGGCGCGTGTTGGGCAAAGGAAAGAATATCATCTCCCTGTGGCCATGGAGGCCCTTGCCCCAGCTCCC
TTTGCCCCGGGGGAGAGTTTCCTGTTAGACCTCATTCTTTCTGGTCAGACTGGGGCCTGGGGAGGTGGTG
ACAGAGGGTGGGGGCTCCAGAAGTGGCCAAATGAGGGGCTCATCCTTGACCTTTTGGTCAGACACAAGCA
CAGGCTTGGCTGTTGAACAGAGTTCAAGATCAGAATAATATGCTCTTGGCTATAGAAAACATGTTTCTCA
AGCAGGTGGTGAGTTTCCAGAGCATCAAATGCAACCTCTGGTACAGATGGCCCCTTCTTTCTGTTTCACT
GGAAATGTGCATTGGTTTGCCCAAGCATTGTTTACTTGCATTTCTTGGGATATCTCCTATTAGCTAAGGT
TAGATGTGTCTGTGCCTTAAGAGGCTAGGCTGGTTCATCATTTGATGGTTGACATGCAGGCAAAGACAAG
CAACTGGTATGGCTGCAATGAGGATTCAGACAGTAGCTGTTCCTTCAGTATTCCAAATGGAAACTGACTT
CCTCCTGGTCTTTGGTCCAGGATGAGAAACTCACTTTTTTTTTTTAATGGGGCTGCTAGACTTCCAAGAG
AGCCATTAACGAAACAAAAACCAAGGCAGTCTCAAAGAGTATGGATTCCATTTGGAAGGCTTTCAAATGG
CATACTGGAGCATCTGAAGAAAATAAACAGATTTAAGGCACAAGCATATTACTTCATCAGGACTGGAAGG
AAACTGGTTCAAGGCTGCCTTTGAACTAAAGCTCTTTAGTTCAATTTCAAAACCATTCAGCAATTCCTAC
GTCAGTTAAACATTCAAAACTAGAAAGTATGGAGTTTAGTTCATTTTAAAAAGACAAAAACAAAAAACTA
GATCCAGCTTTGGATTCCTACAAGTAAAAGCAGTACATTGCTTCATCTCCATGGCAACGAGTGTTGAGTC
TAGTTAATCAACAAATATTCACTGACCTAGGCACCATGCTGCATGTCATTGGGTTTTCAGAGATGATAAC
ACACATCCCTAGAGGACATAGACATGCAAAGTACCACATGTGATGCAAGGTGTTGAGTGCCACCATGGAG
ACAAGGGAGTGAGTTCAAAGTGCTCAGAGACAGCAGGTGGGGGTGGTCTTCTCAGAAGAGATCACAGGAG
CTGGTCCTTTGTAAGTGCAGAGGATGTCTCCAGGCTAAAATGGAAAAAGTGAGAATCCAGGCATAAACAT
TTGCCAAAAGCCATTGAAAATATGGCTAAGTTTCAGAAAGGAAACAGCTGAAAATTGGAGTGACAGGAGA
TGTGACTGGTCTGATAGGTCAGGGTGGGGGGGGACATAAAGAACCTTCCACGCCTAGGTGAGGGGAGCAG
AGTTTATCTTGAGAACAACGAGGATCCACAGGTTTTGTCCAAAGGAGCTTGATGGTCAGGTTTGCGTTTT
AGAAAGATTTACCCTGTCAGTGAGTTTTGGGCATAGATTAGAGTGAGAAAGAATCTAAGAGGAAAAAGGC
CAGTCCAGGTGTGAGAGCCATGATTCAAGAGGAGGGTCCATCCCTGAAGGCCACATCTTTGTTTCACTCT
TTAAATGGAAATTGACAGATATCTAAGAGGCCAGGACGTGTGGGTGCCAGGAAGACCCAACCTGGAGAGA
CTGGGCAGTTCAGTGCAGAGAATACAAAGTAGAAATTAGTAGAAGGGTGGTGCTGGTCCTCTTTCTGCAT
CCCATTTGTTCTATTCCCTGCAGTTACAGCTGCTCAGCCCGCTGCCCATTTATGTGGAGATTGGCTGGGG
GAGGAGGCAGTTTGGCATGGCAGAAATAGCATGGACTTTGGAATCGAGCACGCCCAGATTTTAATCTTCA
GTCCTATGTTTAATAAGTAGGCGCTATTGGGGAACACATTTCAGTTCTCTGAAATTCAGTTTCCCCATTT
GTAAAATGGGGATAACAATGTCTATGTCATAGGGCAGCAGTGAGGATTACAGAAAATTTAGTTGGTGATG
TAACCAAACTGGATTCAGGATAGGCAACTCTGAATTCACCTATGGTCCCCCTTGGAGTGATAGTGACAGA
TTCCCTCAAGTTAGGCATATGCCTTCCCCTACCCCTTGGAAGCATCAAACTCAGTGGCATGAGGAAACAG
GTGGTGGCTTTAGAAGGCAGTATGTTTGGTGACAGTCTCCTTGGATGCTTAAGAGAGAGGCTCAAAGGAG
GATAGTGATATTAAAGTAATTAAGCTGCACTTAACAAGCACTTACAATAGACCAAGTGCTCTACAGTGGT
GTCTTTGGGCCTCCCCGACCAGGTAGCTCCTGTTCCTGCCCTCATTCCTGCTGTGGTTTTAGCCTACAAC
AAAAATAAGCCCTCTTCTCTCATTTCTGGACCTTACAACAAACACTCTCTTTGTGACTTACTGGTTGAAA
GTGGGTTTGATAAGTGTGAAAATATCAGGAAATGGCAGAAAAGGCAATGCCTGGGAAACAGTGTGCATTG
CTGTGAATATTAATTTTCATTTGAATATGAGCTGCCATGGATCTTGACTTCTGAATAAGCTTCGTTGATC
ATAAATGGTGATTGTGGAGTATCAGCTGGGCTATTTACATTACAAAATTCATCCCAAAACTGTCTTTTCT
TAAACCACAGAGAGCTCATGAAAAGGAAATGGCATCTTAGAAGTTTAACTAGGCTTGCTTCCAGGAAAAG
TATTAAGAATCTGGATTAGTATTGGTGGTTTGCACAAACATGGCTTTTTTTTCTCTCATTCTTTTTTTCT
TTGTCCTCCTCCTCTCTGTACCCCCTCATGAAAAACTAGGCGGTGAAGTGGGGCAGGAAATGGTTTCATT
CAGATCACTCTCCTTTCCTGCAGTAAACAGGGGGTTCCCCACAGTGGTTGGCTTTCAAACGTTTTTCAGG
TTCCCTGTTTATCTACCCTAACCATTGCCCTTTGCCAGTGCTGCATCTCTGAAGTAATGCAAGTGCTTTG
AATCTGGTGGGATACCAATGAGTTGATATCTGGTTGCAGTTAATAATGTTTCATAAATAAGAAGCCCCCT
CACCAAACAGAAAGTTAGTGTGGCATTACCATTTTATAAATATCCACTCATTAAATAAGCATCGAGGGCC
CACTATGTACCAAGCATTGTACCAGTCTCTTGGGATGCAAAGATGAGCCAGGCACGCATTCTGTTGCCAA
GAAGTGGGCAGTCTACTAGGAAGTCATAGATTATGATATATAAACTTGATGTATAGTGATTAGTGCTCAG
GCCCTGAAAAGGGACAGATGCAGTGTAGAGTAAGTCCAGATTACTGCTAACTTTTTTGAGGTGAAGGAAG
GTAGAAGTGTTGGTCATGAATGACTTCCCAAAGAGGTAGCATCTAGACATGAGAAGACATAAAGAACATT
CCAGATGAAGGAAAGAGAACAGCAAGGGCATGGCAGTTGAATGCCTCAGTACAGTTTAGGGTTAAAACAA
TTACAGTGGAGTTGCATTGTGCATATATTAAAATCACACATATGATACAAGTAGAAAGAGGAGAAAAATT
AAGAGTGCAGGCTATTGCATTTGATGACTGTATGTCCTGGAGTGAGCATGAATCCACTGTAGGTCATGGT
TTCTCTGCCTCTGGTGATGAGTGGTGAGATGATGTCTTGTGCTTTTGGGAAAGTTAAATGATTTTCAGGG
ATAAAACTAATTCTATGTGATATTTACCTTCTGGCTTGATGTGATGACCTAACCTCAGAGCAAAACGCTT
TTCCTGTACAGAAATAGGGGAAATAAGATTGGAAAGATAGTTTTGAACCAGATGGCAGAGGGAATCTCAA
GTGCTCAAATGCCATTTTGTTGTTCCCCATGGCTTTCTTTTCCCTACTCAGGTCATCTCTCAGGTGAGAA
AATGACTCCTTTCTCGTCTGACTCTAGTCTTTTTCCTGGTCTTATTGAAACACGACCTCTAAGACTGAGA
AGGAGGAAGGAGGGCAGAACCATGGCAGAAAGTAGTGAAACAGTGACACAGCAGGACTAGACAGGTGGCT
GACCACGGCACACCCTCCACCATCTGTCTTTCCAGAGCTCCACTGCTCACTGTAAATGGTTACCTGGTTA
CCCCTGCTTTTGCATCAGATAAAGCCATCAGCTACATTGCAAGGAAAACTAAATATCAAACTTAGCGGGG
CAAAATAGTTAAAAACAAAAATGGTGGGAGAGGGGAGAGGCCACAGGATTTAGAGACCGGAGATCTGAGT
TCAGGTCTTCACCCTGAAATGTGCTGTCTTTGCAATCTTGGACAAGTTCCTTACCCTCTCTGAATCTCAA
TTTCCTTCCTTCTCACAGGGTTAAAGATGTCTACTTTCAGGTTTCTTATAAAAATTGAATGAACTAATAT
GTACTATGCAGAAAAAAAATTAGACAAACCTTCACTGTTATTATTTGTTTTACACACTTCCATGAAGAGC
CAGCTGTCTTGCTAAGAATCCATAAAAGTGTTCATGCATCCCTGCAACTGAATATACACATTGTGTTAGG
GAATTTCTTTTTTTTTTTTTTTTTTTATTATACTCTAAGTTTTAGGGTACATGTGCACATTGTGCAGGTT
AGTTACATATGTATACATGTGCCATGCTGGTGCGCTGCACCCACTAATGTGTCATCTAGCATTAGGTATA
TCTCCCAATGCTATCCCTCCCCCCTCCCCCGACCCCACCACAGTCCCCAGAGTGTGATATTCCCCTTCCT
GTGTCCATGTGATCTCATTGTTCAATTCCCACCTATGAGTGAGAATATGCGGTGTTTGGTTTTTTGTTCT
TGCGATAGTTTACTGAGAATGATGGTTTCCAATTTCATCCATGTCCCTACAAAGGATATGAACTCATCAT
TTTTTATGGCTGCATAGTATTCCATGGTGTATATGTGCCACATTTTCTTAATCCAGTCTATCATTGTTGG
ACATTTGGGTTGGTTCCAAGTCTTTGCTATTGTGAATAGTGCCGCAATAAACATACGTGTGCATGTGTCT
TTATAGCAGCATGATTTATACTCATTTGGGTATATACCCAGTAATGGGATGGCTGGGTCAAATGGTATTT
CTAGTTCTAGATCCCTGAGGAATCGCCACACTGACTTCCACAATGGTTGAACTAGTTTACAGTCCCACCA
ACAGTGTAAAAGTGTTCCTATTTCTCCGCATCCTCTCCAGCACCTGTTGTTTCCTGACTTTTTAATGATT
GCCATTCTAACTGGTGTGAGATGATATCTCATAGTGGTTTTGATTTGCATTTCTCTGATGGCCAGTGATG
ATGAGCATTTCTTCATGTGTTTTTTGGCTGCATAAATGTCTTCTTTTGAGAAGTGTCTGTTCATGTCCTT
CGCCCACTTTTTGATGGGGTTGTTTGTTTTTTTCTTGTAAATTTGTTTGAGTTCATTGTAGATTCTGGAT
ATTAGCCCTTTGTCAGATGAGTAGGTTGCGAAAATTTTCTCCCATGTTGTAGGTTGCCTGTTCACTCTGA
TGGTAGTTTCTTTTGCTGTGCAGAAGCTCTTTAGTTTAATTAGATCCCATTTGTCAATTTTGTCTTTTGT
TGCCATTGCTTTTGGTGTTTTGGACATGAAGTCCTTGCCCACGCCTATGTCCTGAATGGTAATGCCTAGG
TTTTCTTCTAGGGTTTTTATGGTTTTAGGTTTAACGTTTAAATCTTTAATCCATCTTGAATTGATTTTTG
TATAAGGTGTAAGGAAGAGATCCAGTTTCAGCTTTCTACATATGGCTAGCCAGTTTTCCCAGCACCATTT
ATTAAATAGGGAATCCTTTCCCCATTGCTTGTTTTTCTCAGGTTTGTCAAAGATCAGATAGTTGTAGATA
TGCGGCATTATTTCTGAGGGCTCTGTTCTGTTCCATTGATCTATATCTCTGTTTTGGTACCAGTACCATG
CTGTTTTGGTTACTGTAGCCTTGTAGTATAGTTTGAAGTCAGGTAGTGTGATGCCTCCAGCTTTGTTCTT
TTGTCTTAGGATTGACTTGGCAATGCGGGCTCTTTTTTGGTTCCACATGAACTTTAAAGTAGTTTTTTCC
AATTCTGTGAAGAAAGTCATTGGTAGCTTGATGGGGATGGCATTGAATCTGTAAATTACCTTGGGCAGTA
TGGCCATTTTCACGATATTGATTCTTCCTACCCATGAGCATGGAATGTTCTTCCATTTGTTTGTGTCCTC
TTTTAGTTCCTTGAGCAGTGGTTTGTAGTTCTCCTTGAAGAGGTCCTTCACATCCCTTGTAAGTTGGATT
CCTAGGTATTTTATTCTCTTTGAAGCAATTGTGAATGGGAGTTCACCCATGATTTGGCTCTCTGTTTGTC
TGTTGTTGGTGTATAAGAATGCTTGTGATTTTTGTACATTGATTTTGTATCCTGAGACTTTGCTGTGTTA
GGGAATTTCTTTGTGTCTTTCTTTACCAGCTAGACTGTGGGGTTCTTAAGGACAGGGAGAGTCTTATTTA
TCTTTAAAATTACTTTAGCATTTACAAGTATCTGGCATATGACAAATATTCATTGAATGAATGGGCCAAT
TGTGAGTATCAGAAAGTCTCCCAAATTAGAAAAATCTGAATTAATAATTTTTAGCCTACCACTGTTATGA
TAAAAGTATGAATTCTTGAAAATTGCCTTATTTTGATTCAGATTAATTCAGTAAATATTTATTGAGCATC
AACTGTGCACCAGACACACATTCCCTTACCCTCAAAGAGTCTGTGGTCTCACTGGAGAGAGAGACAGACA
CATACTCTACCAAATCAAAGACGGTCAGACTGAAAAGTACCATTTAAATCAGCCTCAACACAGACTGCTG
ACTTGACTTGTCGCTTCCTGACCAGCGTGGGCTCAATGTCTCTGGGACTGCCCATCAGTAAAGGATGTTG
TGTATGACATGAGTGATAGCAATAAGGTGAATTCCCAGAGAAAGGGGTAGAGCTCCGAGTGGGGAGTCCT
GTTTGTTTGTGTTTGAGCTACACATACCTCTCTCTCCAGGGGCCCACCTTTCCTTCCCCTTTGGCTGGAA
CAGCATTGTTGTCACCAAGTTAGCCTTTGCCCTAACACCTGCTCAGAAAGGGGAGTTACTTTTCCCCTTG
GTGGGGCTCCTGATGGTTACTATCATCCCTTTCTTTATCCCCTACTCCCTGCCATGAGGAAGAGAATTTG
ACATTTCTCATATAAGCCCAAGAGGCAGAACTGGGAACTACATATAGCAGCGGTCCCCAGCATTTTTGGC
ACCAGAGACTGGTTTCATGGAAGACAATTTTTGCTTGGACTGGGGTCGGAGGAATGGTTTCGGGATGATT
CAAGCTTATTACATTTATTGCGCACTTTATTTCTATTATTATTACATTGTAATATAAAATTAAATAATTG
TATAACTCACCATAATGTAAAATCGGTGGGAACCCTGAGCTCATTTTCCTGCAACTAGATAGTCCCATCT
GGGGGTGATGGGAGACAGTGACAGATCACCACGCATTAGATTCTCATAAGGAACGTGCAACCTAGATCCC
TCACATGCACAGTTCACAATAGGGTTCACACTCCTATGAGAATCTAATGCCGCCACTCATCTGACAGGAG
GCGGAGCTCAGGTGGTAATGCAAATAACGGGGAGTGGCTGTAAATACAGATGAAGCTTCACTTGCTTGCC
TGCCGCTCACCTCCTGTTGTGTGGCCCAGTTCCTAACAGGCCATGGACCAATACCAATTGGGGACCCCTG
ATATATAGGATGGCAAATTTTAGCTCAATGTAAGAAAAACTTTCACATAGAAAGAACCATTTGAAATTGG
AATAAGCTGCCTTGAGGAGTGAGTTCCTGCTTTGGGGAATGAGGCTTACAGAGACTAGATGAACACTAGG
TGTGGACTCAGTCAAGCATCTTATGGGGACTAGGATTGACTAGAGCAGAGCTTCTCCAGCATTCACATCA
CTGGGCATCTTGTTGCATTGCAGATTCTGGTTAGGTAGGTCTGGGGCAGGGCCTGCCTAAACGGATTCTG
CATTTCCAGCAAGTTCCCCAGTGATGCTGATGCTGCTGGTCTGGGGACCACACCTTAAGTATCTAGGAAT
AAGGGAACCTATTGGTAATTAGGAATCCATGAAATTAATAGCTACCATTTAAAAATACTCAGTGCCAGGC
ACTGTGCCAAGGGCTTTACATGTGTTATTTTGTTTAATGTTGCTTAATAAATAAACTTGGAAGTACCTGA
AATAGATTATAGCAGATACAGGAAGCATGGAGATCAGTCAAGAGCTATTAAAATAATCTAGATAGCAAAG
ATATTGAAGATCCTCATCCAGAGGCCATGGGTATTGAGAGGGAGATATAAATGCAAGGAACATCACAGAG
ATAATAACGGAAGTTCTTGCAATGGATTAAATATGATGGCTCGGGGGGAAGAAATTGTTAGAGATGATTA
TAAGAGTGATGTGAAAGGCTGTAATATCATTAATAGACAAGCTTAGAGACGTGGCTGATTTGAAGGGGAA
GGTAATGGCTTGAGTTTTAGATAGAGAGGATACTTGCAAATATTCACTAAGTGCTCACTATATGCTAGGC
ATGGTTCCAAATACTTGACATGTATTAACTCGTTTACTTCTCATAACAACCCATAAGGCAGCTACCATTA
TTAGCACCATTTTACAAATGTGGAAACAGACCCAAAGTCACCCAGTTAATAAGTGGTCGAGGCCAGGGGT
GGTGGCTCACACCTGTAATCCTAGCACTTTGGGAGGCCAAGGCAGGCGGATCACAAGGTCAAGAGATGGA
CACCATCCTGGCCAACATGGTGAAACCCCTTCTCTACTAAAAATACAAAAATTAGCTGGGCCTTGGGAGG
CCAAACAGGAGAATCACTTGAACCCAGGAGGCGAAGGTTGCAGTGAGCCGAGATTGCATCACTGAACTCC
AGCCTGGCGACAGAGCAAGACTCTGTCTCACAAATAAATAAATAAATAAATAAATAAATAAATAAATAAA
TAAGTGGTAGAGTTCAGGTCTACAGCCAGGAAATCTAATTCCAAAGCTTTGATGTGGTAGGTTTTAGAGT
TTGTAAAGTTCTTTACTGTTGTTAGCTTCTTAATAACTCCCAACCTGTATATTTATTTTTAGCAGCTTAC
ACTTTTAGTGCTTATATAATCATTTCTCACAGATTCTTGATCCTCTATGTATCAATTTAACAGATATTTA
TTGGGTGTCTCCTATGTACTAGGCTTGGTGCTGGAGATACAATGTTGAGCAAATACAGTTCATGCCCCCA
TGGTGCTTACAGTTAATGGGAGGAGAAAAAAATCACATAACTATCTGACTTCAAACTGTTGTGTCCTGCA
AAGGGATAGATTAGAATGCCAGAGGAGAACATGATCTAGAATAGGGGTAAAAAGGGAAGCTTCCTTCAAG
AAATATTTGATCTGAGATCCAAAGAATGAGTATATCTTAACCAGATGAGGATGGGAAAACATGAGCACAG
ACCTAAATATAGCTCTATTATTTCACTCCATCACTCTCTCCCCCTGACTCCTTCTTCCCTGGTTCCACCT
ACCTCGCAACCCCTCCCTCTTGCTCTAAGCAGAGATGTCTATGCCTGGATAGCCAAAATGCACAGAAATC
CAACGGTGTCTTTAGGTGTTCATACCACACAAAGATTAGGAGTGTGATCTATCTGTCGCATCTCTAACCC
CAGTGGTTATAACATCGCTTGCCACAGAATAGGTGTTCAATAAATATTTGTTGAAAGAAGTAGTTAGTTA
ATTTGGTGCACAGAAAATAGGAGAGAGTTATAGAAAAGCTAGTTCATCTCAGGACGAGGAGAAACTTTCT
AACAATTAGAGATGTCCAGAGATGGAAGACATCTTGTTATAAGAGAAATATCTCTGGACTTAGAGTAAAA
GAAAATAGCCCCGTGCTAATTTTGACTATGGGCAAGTCACTTAATTGAGGCTTACCTTCCATACCTATTA
CTTAAAGATAATCTTTGTCCTGCCTAATTCACAGGATTGTTGTGAGAGTGAGATAAGATAATCTAAGTAA
AAGAGTGTTGTATAAAGTGCTATTAAATATATATGTGCTATTATGGCATAATATAGTTAACTCCCAGTCC
AGCCACTACCTGACACTCTCTTTATGGTAGCTTGTAGTAGAGATTCTTGTCTTAGATAGAAATTTGAACT
AAATAACTCAAACCCTTTCCAATCTATATATACAATGATTATGACACATAAGTCTATTACTTAACCATTT
CTGTATGTTACATATCAAAGTTTTGAATATGTAATGCCAGAGTTAAAATACTAAAGCATTCTATGAAAAT
TATATAGATCAAAATGCAAGAAATATTAAGAAGCTTGTTTTGTTTAATGGTGGACAAAGACCTTAAACTA
AAATCTGCTGTGTATAATTCTCTAGGAAGGGGGATAGTCCAAATGCCTGATTTCCCCCCGAATTTATGAA
CTCTGTATATTAGCTCCGTAGCCATGTTTACCAAGCTACAAGAAGAAAGGAGATCCCTTTAACTAAAATG
TCAGTGATTAGGTTTTAAGGGATTTCAAATTACTAGGGAAGGACCCAGACACTTAGTGACTCCCTAGTGA
TTTGAAAATCTTCCTGGCTGCTGGCAGTAGTTTGCATGGAGACTGAATATTATCTCAGCCCTTTTGAAAA
AGTAAAGAAGCTACAAATCCAGTGGCTGTTCAAAACAACAAAGAGGGTTGAGGTGCCTTTGGCTTCTAAC
AGGACTGTCTCTGAATTCCTCAAGGAATTCCAGGCCTAGAGTTGATCCAGCTGGCAGGAGGTATCGATCT
CCACTTGAATTCACTGAATAAATAATGGTTGCCATAGTTATTGAGCAGTCGCAACTGGTTGGAGAGCAGG
GAAAATTCTGCAGTACCTTCTGTGAGTTTGGCCATGAGAAAGTCTGGTTACAAGGTTCCACAATCAGCTC
CCTCCCCAAATCCAGCCAACATATTACTCAGTTCCTGTATCCTGATGATGAATATGAGATCTTGAATGTT
ATGTAAGACATGAGACACTGAAAGGAAGGGAAAGCTTAAATCAATGGACTGGTATTTATTCTGAGAAAGG
CGTCTCTCCCTAGGTTATGAATATAATTGAATATTTTTCATTATTAATTCAATTTAGTATCAGCAAGGTG
ATGTTTGATCATGTTATAGCATTCAAGGGCTTCACAAAGGACGAAATCCATCTCGTTCTATTTTAACATT
ATTATACAAAGAGCACAACCTGGACATTTTAAAAGGAAGGAAAATTTATCCCAAAACCCCATAGGTATTT
TCACGTTTATATTTTCTTTCACCCTTCTCTATATGAAGAAATAATTTTTGCATAGATGGAATCACAGCTT
TAGTATTTAATATTCTACTCTTTATTTAATGTTGTAAACGTTTTTTTTTTTAATTTTGCTTCATGGCCTT
CACATTTGTTCTGTTTGATGTCTTCAGAATCTTCCCTTCCATATACCATTATTTACTTAACAGTTCCCCT
ATTCTTCAGCCCTTTGCCTTTTTCCAGTTTTTTGTTTGTTTGCTTTTGTTTTGTTTTGTCTTGGTAATAG
ATAATGCTGAACCAAATGTCTCCATGTATACAGTTTTATTCTTCTGTTAAATGTTTCCTTTGGATAAATT
CCCAAGAGTGGGATTACTTGGTCAGAGGCTATGAACGATTTATAGCTCTTGAAATTTACTGCCAAATTTA
CAGCCTGTTCTTTTGTTTTGGGGAGAAAATAAAACTTTAGGCAGAACACCACTGCATGCAGGATTCTCTT
AAAATTCATTCTCTTAAAATTCACAACAGTCTCTTCTGAGACTGAAACATGGAGCCTGTGTATTTAGTAA
TGATAAGCCGTAGGAGACAGGTCCGCACCTGGGCTGATTAGCTAATAGTTTCTCAGGGAAAGCAAAATGA
TGCACTGCAAAGCTTGGAAATTAGGCCACGAATTGCTTTTTGTTAGACCTTGTAGTTACCAGCCACTTGC
CCACTGGCAAGCATTGTCTTTGGTTCTGATTATTCCATGTGTATAGCACTCTACAGCTGACAGACCACTT
TTCCATAACATTATCTTAATTGAGCCACAATAATCCATGGAGTTGGGTTTTATTATCATTGTACTTTTTC
TTTTAATTTATTACATAACATTGAAGACACACAGGAATAATAACAGCAAAGGCTGTGTATCTGCCACTCA
ATAATTCCGATTTTCCTGTGAAGACATTGAGCCTTAGAGAAATTAGGTCACCCAGCTAGTAAATGACAGA
ACCAGAACTAGAACCTAGGTCTGTCTAACTCCAGGGCCAGTGCCCATCTCTTTATAGCATATTGCATTGA
GACAAGATGCCTGCAACTTACAGTCCATGGAGATCCCGCTGGCAATCTTCATGGTTGTATTTTAATATCA
TTATAACATATCAGGCCAGGTGCAGTGGTGCATGCCTGTAAGCCCAGCACTTTGGGAGGCCAAGGTGGGT
GGCTCACTTGAGGCCAGGAGTTTGAGGCCAGCCTGGCTGATATAGTGAAACCCTGTCTCTACTAAAAATA
CAAAAATTAGCCAGGTGTGGTGGCACACACTTGTAATCCCAGCTACTTGGGATGCTGAGGCAGGAGGATC
TTTTGAAACCGGGAATGTTGCAGTGAGCTGAGATTTCACCACTGCATAGCCTGAGTGACAGAGTGAGACT
CTGTCAAGGAAGGAAGGAAGGAAGGGAGGGAGGGAAGGAAGGAAGGAGGACACATCAGAGCTTTGGGACC
TTACAGATAATGAAGTTCAACCGTCTCCTTTTACAGGGCCCCAAAGTGGGAGGAACTTCCCCAAATCATA
CACAACTAGCCAAACTGAGGATAATGCAAATTTTCTGGTTCCCAGGCCTCTTATTTGGGACAAGAAAAAA
GTGTCTGGATAGTTCTTCATTTTTCTTATTGGCTTTTGCTTCAAGGAAATAACTTTTCTAACAAAAATGC
CCCCAACTTAAAAAATTAATTTAGAGCCAGGCACGGTAGCTCATGCCTGTAATTTCAGTGCTTTGGGAAA
CTGAGGCAGGAGGATCACTTGAGCCCAAGAGTTCAAGACCAGCCTGGGCAACATAGTGAGGCCCTGCCTC
TACAAAAAATAATTTAAAAATCAACCAGGTATGGTGGTGCATGCCTATAGACCTAGCTACTTGGGAGGCT
GAGGCAGGAGGATCACTTAAGCCCAGGAGTTCCAGGCTACAGTGAACCATGACTGCACCACTGCACTGCA
GCCCAGGCAAGAGAGCAAGACCATGACTCAAAAAAAAAAAAAATTAATTTTGTAAAAATTAGAAACTATT
GCTTGATTTCCCATTAGAAGGATGTCTTCCATCTCTCAAGAATTATGATTTTACATGTAAATGTACTGAG
AGAAGAAAATTTCGAGATAACAGAGGCCAAGCTCTTCCTTTAGAAATGAGGCTCTGAGGCCAGGCACAGT
GGCTCATGCCTGTAATCCCAACACTTTGGGAGGCCAAGATGGGAGGATTACTTGAGGTCAGGAGTTCGAG
ACCATCCTGGCCAATATGGTGAAACCCCATCTCTACTGAAAATACAAAAAATTAGCTGGGCATGGTGGTA
CACACCTGTAGTCTCAGCTACTCGGGAGGGTGAGGCAGGAGAATCGCTTGAATCGGGGTGGAGGAAGTGG
GGGAGCAGTGAGCCAAGATCGCACCACTGCACCCCAGCCTAGGCAACAGAGTAAGACTCTGTCTCAAAAA
AAAAAAAAAAAAAAAAAAAAAAAAAAAAAGAGAGAGAAATGAGGCTCTGAAGTCTAAATGTTGAGGAAAA
GCATAGTGATTTGTTGTGGATGTCAGGCTCCCTTCTCATTGCTTTGGAACAAGAGACCAGTATTTGCCTC
AGAAAACTGGGTTCTAATCCTAGCTTTTAGCAGCTGAGTGACTTTGGGAAATTCATTTAACCTTATACAT
AAGATAGATCATAGTATTTAACATGGAGATCATTTTATATATTGTTTTGTAAAATATAAGATACTGGTAT
TTGTGTCTTAATACATTATAACATGCCTGCACCACATCCTTCTGGGCTTCCTCCCCTTCTCTTTGGCTTT
TCCATCTCCACAGCAGGAGGACATGTGTATTTTGTACAGAGTGCCAGGTGTAGGTCAACAGGGGTTTCAA
GTGGGGGGGAACAAAACAAGGAAAAGAAAAGCTGAATTCTTCTCAGTGCTGGTTAGCATGAAAACACAGG
AATGCACTTTGCTGTGTTGGGTCTTTTCGAGCAGCCCTTGCATTAGCAAATGGAAGTTACAGCCTCCCCA
CCTCCCTTAAATGAGTCTGTCAAGATAGAAACATTAATTCATTTGCCTGGATGTGTTTCTATGTCACTCT
GAAAAATAGTTGCCTACTGACAAGGCTTTGTGAGAGCTTAGTAGGATAATGTATGCAAAATAATAACCAC
CCTGCCATTCATTCCATGTGGCTGTGTGCCAGGAACTGTGCTTCCCTTGTGTTACTTACCTAAGCTGGCC
ATCCCAGAAACCCTGAATTGGGTATGATCATTTTCTTCATCTCATAAGGAAATGGGTGCTCAGAAGTAAA
GAGACTTACTCTAGGGCACACAGCAAGTAAGTGTTGGGACTGGGATTCAAACCTTGGTGTTGCTTCATAC
CAAAGGCCATGAGCTATCTCTAACAGGGAGGGCACTTTGATTGCAAAGAAGCTATCCAAACTGGCAGAAT
GAGACACAGACCTCAATTAGGGATTCAAAGTGTCTTAAGATGTATTTGCCTCCTAAACTTCTTGCCCTTC
TCTATCCCTCTGCAGAAAGACATGGGTTAAAAGAACCAAAGAGAGTCGAAGAGCTATGCAACAAGATCAC
AAGCAGTTTAAAAGACCACCAGAGTAAGGGACAGGCTCTGGAGCCCACCGAGTCCAAGGTCCTGGGTGCC
CTGGTAGAACTGAGGAAGATCTGCACCCTGGGCCTCCAGCGCATCTTCTACCTGAAGCTGGAAGACTTGG
TGTCTCCACCTTCCATCATTGACAAGCTCTTCCTGGACACCCTACCTTTCTAATCAGGAGCAGTGGAGCA
GTGAGCTGCCTCCTCTCCTAGCACCTGCTTGCTACGCAGCAAAGGGATAGGTTTGGAAACCTATCATTTC
CTGTCCTTCCTTAAGAGGAAAAGCAGCTCCTGTAGAAAGCAAAGACTTTCTTTTTTTTCTGGCTCTTTTC
CTTACAACCTAAAGCCAGAAAACTTGCAGAGTATTGTGTTGGGGTTGTGTTTTATATTTAGGCATTGGGG
GATGGGGTGGGAGGGGGTTATAGTTCATGAGGGTTTTCTAAGAAATTGCTAACAAAGCACTTTTGGACAA
TGCTATCCCAGCAGGAAAAAAAAGGATAATATAACTGTTTTAAAACTCTTTCTGGGGAATCCAATTATAG
TTGCTTTGTATTTAAAAACAAGAACAGCCAAGGGTTGTTCGCCAGGGTAGGATGTGTCTTAAAGATTGGT
CCCTTGAAAATATGCTTCCTGTATCAAAGGTACGTATGTGGTGCAAACAAGGCAGAAACTTCCTTTTAAT
TTCCTTCTTCCTTTATTTTAACAAATGGTGAAAGATGGAGGATTACCTACAAATCAGACATGGCAAAACA
ATAATGGCTGTTTGCTTCCATAAACAAGTGCAATTTTTTAAAGTGCTGTCTTACTAAGTCTTGTTTATTA
ACTCTCCTTTATTCTATATGGAAATAAAAAGGAGGCAGTCATGTTAGCAAATGACACGTTAATATCCCTA
GCAGAGGCTGTGTTCACCTTCCCTGTCGATCCCTTCTGAGGTATGGCCCATCCAAGACTTTTAGGCCATT
CTTGATGGAACCAGATCCCTGCCCTGACTGTCCAGCTATCCTGAAAGTGGATCAGATTATAAACTGGATT
ACATGTAACTGTTTTGGTTGTGTTCTATCAACCCCACCAGAGTTCCCTAAACTTGCTTCAGTTATAGTAA
CTGACTGGTATATTCATTCAGAAGCGCCATAAGTCAGTTGAGTATTTGATCCCTAGATAAGAACATGCAA
ATCAGCAGGAACTGGTCATACAGGGTAAGCACCAGGGACAATAAGGATTTTTATAGATATAATTTAATTT
TTGTTATTGGTTAAGGAGACAATTTTGGAGAGCAAGCAAATCTTTTTAAAAAATAGTATGAATGTGAATA
CTAGAAAAGATTTAAAAAATAGTATGAGTGTGAGTACTAGGAAGGATTAGTGGGCTGCGTTTCAACATTC
CGTGTTCGTACTCCCTTTTGTATGTTTCTACTGTTAATGCCATATTACTATGAGATAATTTGTTGCATAG
TGTCCTTATTTGTATAAACATTTGTATGCACGTTATATTGTAATAGCTTTGCCTGTATTTATTGCAAGAC
CACCAGCTCCTGGAAGCTGAGTTACAGAGTAATTAAATGGGGTGTTCACAGTGACTTGGATACACCAATT
AGAAATTAAATAAGCAAATATATATATATATATAAATATAGCAGGTTACATATATATATTTATAATGTGT
CTTTTTATTAACCATTTGTACAATAAATGTCACTTCCCATGCCGTTATTTTATGGTTCATTTGCAGTGAC
TTTTAAGGCAGTACTGTTTAGCACTTTGATATTAAAATTTTGCTTATGTTTTGCTAAATTCGAATAATGT
TTGAAGATTTTTAGGTCTAAAAGTCTTTATATTATATACTCTGTATCAAGTCAAAATATCTTTGGCCATT
TTGCTAAGAAACAAACTTTGAATGTCAAACTGATGTCACAGTAGTTTTTGTTAGCTTTAAATCATTTTTG
CTTTAGTCTTTTTAAAGGAAAATAACAAAACTATGCTGTTTATATTGTCATTAAATTATACAATCAAACA
AATGCCAAATGAATTGCCTAATTGCTGCAAAGTATAACCCAGATAGGAAATCATATGTTTTTTTCCAAGA
GTCATTCTAATATTTGATTATGTTATGTGTGCTTTTATGAAAGATTGTTATTTTTATATATCAAGATGAT
AGAACCTGGAATGTTAGGATTTTGAAATGTTAGACTTGGAAGGGGCCTGGTCTGTCAACTAGTCCAACCC
CTTAAAATTCATAGAGGAGCAAACTGGGGCCCATTGAAGGGTGAAGAGTTACTCAAGGTCAAACAGCTGG
TAACAGAATCAAGACTAAGACCTAATTTACCTTTCCATACTCTTTTTTTTTCTCAACTTCATCTATATAA
AATCAGGCTTTTAAACATAACCACTAATATTTACCTGAAGATAACCATGAGTAAAGTATACTTTTGCATT
AATTTTTTGAGCTTATATGCAAACATAATAAATATTATTAAATATCAGGAAAGCTAACATTTCATACAAG
ATAGCTTCAGACCAAATTCAAATTGAATTTGAATAAATTAGAAATACTGTGCATACATAACCTTCTTGTG
CACCATGAGTATTTGGAAAGTTAATCCTTGTTTTTGTCGTGTCTATAAAGGAAGAACAAAACAAAATAAA
AACAGAGCCCTAGAGAAATGCTGTTACTTTTTATTTTTACACCCATCAGATTTAAGGAAAAGACTTTTTA
GCCATTATAATCTAGTGGTTGGAAGGAATGAAGAAGCTTTTTTAGTAATAGGTCCAGATATGAGTGCTAA
AAATAAAGATGATAGCATGTTCTTCTGTCTTCCATAGTTATTACAACTATGAGAGCCTCCCAAGTCATCT
TATCAACTCAACTCCCTTTTTTTTGTCTTAATGTTGCACATAAGTTTATACAGAGTGGATGACCACACTA
GCACAGAAGAGAACAACATGTATTAAAGCAGGTGATTCCTCCCCTTGGCGGGAGAGCTCTCTCAGTGTGA
ACATGCCTTCTGTGGGCGGAAATCAGGAAGCCACCAGCTGTTAATGGAGAGTGCCTTGCTTTTATTTCAG
ACAGCAGAGTTTTCCAAAGTTTCTCTGCTCCTCTAACAGCATTGCTCTTTAGTGTGTGTTAACCTGTGGT
TTGAAAGAAATGCTCTTGTACATTAACAATGTAAATTTAAATGATTAAATTACATTTTATCAATGGCTA
SEQ ID NO: 10 (NM_006981.4, NR4A3 isoform a mRNA):
GCGCAGCCGGGAGAGCGGAGTCTCCTGCCTCCCGCCCCCCACCCCTCCAGCTCCTGCTCCTCCTCCGCTC
CCCATACACAGACGCGCTCACACCCGCTCCCTCACTCGCACACACAGACACAAGCGCGCACACAGGCTCC
GCACACACACTTCGCTCTCCCGCGCGCTCACACCCCTCTTGCCCTGAGCCCTTGCCGGTGCAGCGCGGCG
CCGCAGCTGGACGCCCCTCCCGGGCTCACTTTGCAACGCTGACGGTGCCGGCAGTGGCCGTGGAGGTGGG
AACAGCGGCGGCATCCTCCCCCCTGGTCACAGCCCAAGCCAGGACGCCCGCGGAACCTCTCGGCTGTGCT
CTCCCATGAGTCGGGATCGCAGCATCCCCCACCAGCCGCTCACCGCCTCCGGGAGCCGCTGGGCTTGTAC
ACCGCAGCCCTTCCGGGACAGCAGCTGTGACTCCCCCCCAGTGCAGATTTCGGGACAGCTCTCTAGAAAC
TCGCTCTAAAGACGGAACCGCCACAGCACTCAAAGCCCACTGCGGAAGAGGGCAGCCCGGCAAGCCCGGG
CCCTGAGCCTGGACCCTTAGCGGTGCCGGGCAGCACTGCCGGCGCTTCGCCTCGCCGGACGTCCGCTCCT
CCTACACTCTCAGCCTCCGCTGGAGAGACCCCCAGCCCCACCATTCAGCGCGCAAGATACCCTCCAGATA
TGCCCTGCGTCCAAGCCCAATATAGCCCTTCCCCTCCAGGTTCCAGTTATGCGGCGCAGACATACAGCTC
GGAATACACCACGGAGATCATGAACCCCGACTACACCAAGCTGACCATGGACCTTGGCAGCACTGAGATC
ACGGCTACAGCCACCACGTCCCTGCCCAGCATCAGTACCTTCGTGGAGGGCTACTCGAGCAACTACGAAC
TCAAGCCTTCCTGCGTGTACCAAATGCAGCGGCCCTTGATCAAAGTGGAGGAGGGGGGGGCGCCCAGCTA
CCATCACCATCACCACCACCACCACCACCACCACCACCATCACCAGCAGCAGCATCAGCAGCCATCCATT
CCTCCAGCCTCCAGCCCGGAGGACGAGGTGCTGCCCAGCACCTCCATGTACTTCAAGCAGTCCCCACCGT
CCACCCCCACCACGCCGGCCTTCCCCCCGCAGGCGGGGGCGTTATGGGACGAGGCACTGCCCTCGGCGCC
CGGCTGCATCGCACCCGGCCCGCTGCTGGACCCGCCGATGAAGGCGGTCCCCACGGTGGCCGGCGCGCGC
TTCCCGCTCTTCCACTTCAAGCCCTCGCCGCCGCATCCCCCCGCGCCCAGCCCGGCCGGCGGCCACCACC
TCGGCTACGACCCGACGGCCGCTGCCGCGCTCAGCCTGCCGCTGGGAGCCGCAGCCGCCGCGGGCAGCCA
GGCCGCCGCGCTTGAGAGCCACCCGTACGGGCTGCCGCTGGCCAAGAGGGCGGCCCCGCTGGCCTTCCCG
CCTCTCGGCCTCACGCCCTCCCCTACCGCGTCCAGCCTGCTGGGCGAGAGTCCCAGCCTGCCGTCGCCGC
CCAGCAGGAGCTCGTCGTCTGGCGAGGGCACGTGTGCCGTGTGCGGGGACAACGCCGCCTGCCAGCACTA
CGGCGTGCGAACCTGCGAGGGCTGCAAGGGCTTTTTCAAGAGAACAGTGCAGAAAAATGCAAAATATGTT
TGCCTGGCAAATAAAAACTGCCCAGTAGACAAGAGACGTCGAAACCGATGTCAGTACTGTCGATTTCAGA
AGTGTCTCAGTGTTGGAATGGTAAAAGAAGTTGTCCGTACAGATAGTCTGAAAGGGAGGAGAGGTCGTCT
GCCTTCCAAACCAAAGAGCCCATTACAACAGGAACCTTCTCAGCCCTCTCCACCTTCTCCTCCAATCTGC
ATGATGAATGCCCTTGTCCGAGCTTTAACAGACTCAACACCCAGAGATCTTGATTATTCCAGATACTGTC
CCACTGACCAGGCTGCTGCAGGCACAGATGCTGAGCATGTGCAACAATTCTACAACCTCCTGACAGCCTC
CATTGATGTATCCAGAAGCTGGGCAGAAAAGATTCCGGGATTTACTGATCTCCCCAAAGAAGATCAGACA
TTACTTATTGAATCAGCCTTTTTGGAGCTGTTTGTCCTCAGACTTTCCATCAGGTCAAACACTGCTGAAG
ATAAGTTTGTGTTCTGCAATGGACTTGTCCTGCATCGACTTCAGTGCCTTCGTGGATTTGGGGAGTGGCT
CGACTCTATTAAAGACTTTTCCTTAAATTTGCAGAGCCTGAACCTTGATATCCAAGCCTTAGCCTGCCTG
TCAGCACTGAGCATGATCACAGAAAGACATGGGTTAAAAGAACCAAAGAGAGTCGAAGAGCTATGCAACA
AGATCACAAGCAGTTTAAAAGACCACCAGAGTAAGGGACAGGCTCTGGAGCCCACCGAGTCCAAGGTCCT
GGGTGCCCTGGTAGAACTGAGGAAGATCTGCACCCTGGGCCTCCAGCGCATCTTCTACCTGAAGCTGGAA
GACTTGGTGTCTCCACCTTCCATCATTGACAAGCTCTTCCTGGACACCCTACCTTTCTAATCAGGAGCAG
TGGAGCAGTGAGCTGCCTCCTCTCCTAGCACCTGCTTGCTACGCAGCAAAGGGATAGGTTTGGAAACCTA
TCATTTCCTGTCCTTCCTTAAGAGGAAAAGCAGCTCCTGTAGAAAGCAAAGACTTTCTTTTTTTTCTGGC
TCTTTTCCTTACAACCTAAAGCCAGAAAACTTGCAGAGTATTGTGTTGGGGTTGTGTTTTATATTTAGGC
ATTGGGGGATGGGGTGGGAGGGGGTTATAGTTCATGAGGGTTTTCTAAGAAATTGCTAACAAAGCACTTT
TGGACAATGCTATCCCAGCAGGAAAAAAAAGGATAATATAACTGTTTTAAAACTCTTTCTGGGGAATCCA
ATTATAGTTGCTTTGTATTTAAAAACAAGAACAGCCAAGGGTTGTTCGCCAGGGTAGGATGTGTCTTAAA
GATTGGTCCCTTGAAAATATGCTTCCTGTATCAAAGGTACGTATGTGGTGCAAACAAGGCAGAAACTTCC
TTTTAATTTCCTTCTTCCTTTATTTTAACAAATGGTGAAAGATGGAGGATTACCTACAAATCAGACATGG
CAAAACAATAATGGCTGTTTGCTTCCATAAACAAGTGCAATTTTTTAAAGTGCTGTCTTACTAAGTCTTG
TTTATTAACTCTCCTTTATTCTATATGGAAATAAAAAGGAGGCAGTCATGTTAGCAAATGACACGTTAAT
ATCCCTAGCAGAGGCTGTGTTCACCTTCCCTGTCGATCCCTTCTGAGGTATGGCCCATCCAAGACTTTTA
GGCCATTCTTGATGGAACCAGATCCCTGCCCTGACTGTCCAGCTATCCTGAAAGTGGATCAGATTATAAA
CTGGATTACATGTAACTGTTTTGGTTGTGTTCTATCAACCCCACCAGAGTTCCCTAAACTTGCTTCAGTT
ATAGTAACTGACTGGTATATTCATTCAGAAGCGCCATAAGTCAGTTGAGTATTTGATCCCTAGATAAGAA
CATGCAAATCAGCAGGAACTGGTCATACAGGGTAAGCACCAGGGACAATAAGGATTTTTATAGATATAAT
TTAATTTTTGTTATTGGTTAAGGAGACAATTTTGGAGAGCAAGCAAATCTTTTTAAAAAATAGTATGAAT
GTGAATACTAGAAAAGATTTAAAAAATAGTATGAGTGTGAGTACTAGGAAGGATTAGTGGGCTGCGTTTC
AACATTCCGTGTTCGTACTCCCTTTTGTATGTTTCTACTGTTAATGCCATATTACTATGAGATAATTTGT
TGCATAGTGTCCTTATTTGTATAAACATTTGTATGCACGTTATATTGTAATAGCTTTGCCTGTATTTATT
GCAAGACCACCAGCTCCTGGAAGCTGAGTTACAGAGTAATTAAATGGGGTGTTCACAGTGACTTGGATAC
ACCAATTAGAAATTAAATAAGCAAATATATATATATATATAAATATAGCAGGTTACATATATATATTTAT
AATGTGTCTTTTTATTAACCATTTGTACAATAAATGTCACTTCCCATGCCGTTATTTTATGGTTCATTTG
CAGTGACTTTTAAGGCAGTACTGTTTAGCACTTTGATATTAAAATTTTGCTTATGTTTTGCTAAATTCGA
ATAATGTTTGAAGATTTTTAGGTCTAAAAGTCTTTATATTATATACTCTGTATCAAGTCAAAATATCTTT
GGCCATTTTGCTAAGAAACAAACTTTGAATGTCAAACTGATGTCACAGTAGTTTTTGTTAGCTTTAAATC
ATTTTTGCTTTAGTCTTTTTAAAGGAAAATAACAAAACTATGCTGTTTATATTGTCATTAAATTATACAA
TCAAACAAATGCCAAATGAATTGCCTAATTGCTGCAAAGTATAACCCAGATAGGAAATCATATGTTTTTT
TCCAAGAGTCATTCTAATATTTGATTATGTTATGTGTGCTTTTATGAAAGATTGTTATTTTTATATATCA
AGATGATAGAACCTGGAATGTTAGGATTTTGAAATGTTAGACTTGGAAGGGGCCTGGTCTGTCAACTAGT
CCAACCCCTTAAAATTCATAGAGGAGCAAACTGGGGCCCATTGAAGGGTGAAGAGTTACTCAAGGTCAAA
CAGCTGGTAACAGAATCAAGACTAAGACCTAATTTACCTTTCCATACTCTTTTTTTTTCTCAACTTCATC
TATATAAAATCAGGCTTTTAAACATAACCACTAATATTTACCTGAAGATAACCATGAGTAAAGTATACTT
TTGCATTAATTTTTTGAGCTTATATGCAAACATAATAAATATTATTAAATATCAGGAAAGCTAACATTTC
ATACAAGATAGCTTCAGACCAAATTCAAATTGAATTTGAATAAATTAGAAATACTGTGCATACATAACCT
TCTTGTGCACCATGAGTATTTGGAAAGTTAATCCTTGTTTTTGTCGTGTCTATAAAGGAAGAACAAAACA
AAATAAAAACAGAGCCCTAGAGAAATGCTGTTACTTTTTATTTTTACACCCATCAGATTTAAGGAAAAGA
CTTTTTAGCCATTATAATCTAGTGGTTGGAAGGAATGAAGAAGCTTTTTTAGTAATAGGTCCAGATATGA
GTGCTAAAAATAAAGATGATAGCATGTTCTTCTGTCTTCCATAGTTATTACAACTATGAGAGCCTCCCAA
GTCATCTTATCAACTCAACTCCCTTTTTTTTGTCTTAATGTTGCACATAAGTTTATACAGAGTGGATGAC
CACACTAGCACAGAAGAGAACAACATGTATTAAAGCAGGTGATTCCTCCCCTTGGCGGGAGAGCTCTCTC
AGTGTGAACATGCCTTCTGTGGGCGGAAATCAGGAAGCCACCAGCTGTTAATGGAGAGTGCCTTGCTTTT
ATTTCAGACAGCAGAGTTTTCCAAAGTTTCTCTGCTCCTCTAACAGCATTGCTCTTTAGTGTGTGTTAAC
CTGTGGTTTGAAAGAAATGCTCTTGTACATTAACAATGTAAATTTAAATGATTAAATTACATTTTATCAA
TGGC
SEQ ID NO: 11 (NM_173200.3, NR4A3 isoform b mRNA):
GCGCAGCCGGGAGAGCGGAGTCTCCTGCCTCCCGCCCCCCACCCCTCCAGCTCCTGCTCCTCCTCCGCTC
CCCATACACAGACGCGCTCACACCCGCTCCCTCACTCGCACACACAGACACAAGCGCGCACACAGGCTCC
GCACACACACTTCGCTCTCCCGCGCGCTCACACCCCTCTTGCCCTGAGCCCTTGCCGGTGCAGCGCGGCG
CCGCAGCTGGACGCCCCTCCCGGGCTCACTTTGCAACGCTGACGGTGCCGGCAGTGGCCGTGGAGGTGGG
AACAGCGGCGGCATCCTCCCCCCTGGTCACAGCCCAAGCCAGGACGCCCGCGGAACCTCTCGGCTGTGCT
CTCCCATGAGTCGGGATCGCAGCATCCCCCACCAGCCGCTCACCGCCTCCGGGAGCCGCTGGGCTTGTAC
ACCGCAGCCCTTCCGGGACAGCAGCTGTGACTCCCCCCCAGTGCAGATTTCGGGACAGCTCTCTAGAAAC
TCGCTCTAAAGACGGAACCGCCACAGCACTCAAAGCCCACTGCGGAAGAGGGCAGCCCGGCAAGCCCGGG
CCCTGAGCCTGGACCCTTAGCGGTGCCGGGCAGCACTGCCGGCGCTTCGCCTCGCCGGACGTCCGCTCCT
CCTACACTCTCAGCCTCCGCTGGAGAGACCCCCAGCCCCACCATTCAGCGCGCAAGATACCCTCCAGGCC
CTCATCACCTTTTTTCAAGTCAAGATTTCATCCCATACATGCATGACTCAATCAGATTTGGAAATGTGGA
TATGCCCTGCGTCCAAGCCCAATATAGCCCTTCCCCTCCAGGTTCCAGTTATGCGGCGCAGACATACAGC
TCGGAATACACCACGGAGATCATGAACCCCGACTACACCAAGCTGACCATGGACCTTGGCAGCACTGAGA
TCACGGCTACAGCCACCACGTCCCTGCCCAGCATCAGTACCTTCGTGGAGGGCTACTCGAGCAACTACGA
ACTCAAGCCTTCCTGCGTGTACCAAATGCAGCGGCCCTTGATCAAAGTGGAGGAGGGGGGGGCGCCCAGC
TACCATCACCATCACCACCACCACCACCACCACCACCACCATCACCAGCAGCAGCATCAGCAGCCATCCA
TTCCTCCAGCCTCCAGCCCGGAGGACGAGGTGCTGCCCAGCACCTCCATGTACTTCAAGCAGTCCCCACC
GTCCACCCCCACCACGCCGGCCTTCCCCCCGCAGGCGGGGGCGTTATGGGACGAGGCACTGCCCTCGGCG
CCCGGCTGCATCGCACCCGGCCCGCTGCTGGACCCGCCGATGAAGGCGGTCCCCACGGTGGCCGGCGCGC
GCTTCCCGCTCTTCCACTTCAAGCCCTCGCCGCCGCATCCCCCCGCGCCCAGCCCGGCCGGCGGCCACCA
CCTCGGCTACGACCCGACGGCCGCTGCCGCGCTCAGCCTGCCGCTGGGAGCCGCAGCCGCCGCGGGCAGC
CAGGCCGCCGCGCTTGAGAGCCACCCGTACGGGCTGCCGCTGGCCAAGAGGGCGGCCCCGCTGGCCTTCC
CGCCTCTCGGCCTCACGCCCTCCCCTACCGCGTCCAGCCTGCTGGGCGAGAGTCCCAGCCTGCCGTCGCC
GCCCAGCAGGAGCTCGTCGTCTGGCGAGGGCACGTGTGCCGTGTGCGGGGACAACGCCGCCTGCCAGCAC
TACGGCGTGCGAACCTGCGAGGGCTGCAAGGGCTTTTTCAAGAGAACAGTGCAGAAAAATGCAAAATATG
TTTGCCTGGCAAATAAAAACTGCCCAGTAGACAAGAGACGTCGAAACCGATGTCAGTACTGTCGATTTCA
GAAGTGTCTCAGTGTTGGAATGGTAAAAGAAGTTGTCCGTACAGATAGTCTGAAAGGGAGGAGAGGTCGT
CTGCCTTCCAAACCAAAGAGCCCATTACAACAGGAACCTTCTCAGCCCTCTCCACCTTCTCCTCCAATCT
GCATGATGAATGCCCTTGTCCGAGCTTTAACAGACTCAACACCCAGAGATCTTGATTATTCCAGATACTG
TCCCACTGACCAGGCTGCTGCAGGCACAGATGCTGAGCATGTGCAACAATTCTACAACCTCCTGACAGCC
TCCATTGATGTATCCAGAAGCTGGGCAGAAAAGATTCCGGGATTTACTGATCTCCCCAAAGAAGATCAGA
CATTACTTATTGAATCAGCCTTTTTGGAGCTGTTTGTCCTCAGACTTTCCATCAGGTCAAACACTGCTGA
AGATAAGTTTGTGTTCTGCAATGGACTTGTCCTGCATCGACTTCAGTGCCTTCGTGGATTTGGGGAGTGG
CTCGACTCTATTAAAGACTTTTCCTTAAATTTGCAGAGCCTGAACCTTGATATCCAAGCCTTAGCCTGCC
TGTCAGCACTGAGCATGATCACAGAAAGACATGGGTTAAAAGAACCAAAGAGAGTCGAAGAGCTATGCAA
CAAGATCACAAGCAGTTTAAAAGACCACCAGAGTAAGGGACAGGCTCTGGAGCCCACCGAGTCCAAGGTC
CTGGGTGCCCTGGTAGAACTGAGGAAGATCTGCACCCTGGGCCTCCAGCGCATCTTCTACCTGAAGCTGG
AAGACTTGGTGTCTCCACCTTCCATCATTGACAAGCTCTTCCTGGACACCCTACCTTTCTAATCAGGAGC
AGTGGAGCAGTGAGCTGCCTCCTCTCCTAGCACCTGCTTGCTACGCAGCAAAGGGATAGGTTTGGAAACC
TATCATTTCCTGTCCTTCCTTAAGAGGAAAAGCAGCTCCTGTAGAAAGCAAAGACTTTCTTTTTTTTCTG
GCTCTTTTCCTTACAACCTAAAGCCAGAAAACTTGCAGAGTATTGTGTTGGGGTTGTGTTTTATATTTAG
GCATTGGGGGATGGGGTGGGAGGGGGTTATAGTTCATGAGGGTTTTCTAAGAAATTGCTAACAAAGCACT
TTTGGACAATGCTATCCCAGCAGGAAAAAAAAGGATAATATAACTGTTTTAAAACTCTTTCTGGGGAATC
CAATTATAGTTGCTTTGTATTTAAAAACAAGAACAGCCAAGGGTTGTTCGCCAGGGTAGGATGTGTCTTA
AAGATTGGTCCCTTGAAAATATGCTTCCTGTATCAAAGGTACGTATGTGGTGCAAACAAGGCAGAAACTT
CCTTTTAATTTCCTTCTTCCTTTATTTTAACAAATGGTGAAAGATGGAGGATTACCTACAAATCAGACAT
GGCAAAACAATAATGGCTGTTTGCTTCCATAAACAAGTGCAATTTTTTAAAGTGCTGTCTTACTAAGTCT
TGTTTATTAACTCTCCTTTATTCTATATGGAAATAAAAAGGAGGCAGTCATGTTAGCAAATGACACGTTA
ATATCCCTAGCAGAGGCTGTGTTCACCTTCCCTGTCGATCCCTTCTGAGGTATGGCCCATCCAAGACTTT
TAGGCCATTCTTGATGGAACCAGATCCCTGCCCTGACTGTCCAGCTATCCTGAAAGTGGATCAGATTATA
AACTGGATTACATGTAACTGTTTTGGTTGTGTTCTATCAACCCCACCAGAGTTCCCTAAACTTGCTTCAG
TTATAGTAACTGACTGGTATATTCATTCAGAAGCGCCATAAGTCAGTTGAGTATTTGATCCCTAGATAAG
AACATGCAAATCAGCAGGAACTGGTCATACAGGGTAAGCACCAGGGACAATAAGGATTTTTATAGATATA
ATTTAATTTTTGTTATTGGTTAAGGAGACAATTTTGGAGAGCAAGCAAATCTTTTTAAAAAATAGTATGA
ATGTGAATACTAGAAAAGATTTAAAAAATAGTATGAGTGTGAGTACTAGGAAGGATTAGTGGGCTGCGTT
TCAACATTCCGTGTTCGTACTCCCTTTTGTATGTTTCTACTGTTAATGCCATATTACTATGAGATAATTT
GTTGCATAGTGTCCTTATTTGTATAAACATTTGTATGCACGTTATATTGTAATAGCTTTGCCTGTATTTA
TTGCAAGACCACCAGCTCCTGGAAGCTGAGTTACAGAGTAATTAAATGGGGTGTTCACAGTGACTTGGAT
ACACCAATTAGAAATTAAATAAGCAAATATATATATATATATAAATATAGCAGGTTACATATATATATTT
ATAATGTGTCTTTTTATTAACCATTTGTACAATAAATGTCACTTCCCATGCCGTTATTTTATGGTTCATT
TGCAGTGACTTTTAAGGCAGTACTGTTTAGCACTTTGATATTAAAATTTTGCTTATGTTTTGCTAAATTC
GAATAATGTTTGAAGATTTTTAGGTCTAAAAGTCTTTATATTATATACTCTGTATCAAGTCAAAATATCT
TTGGCCATTTTGCTAAGAAACAAACTTTGAATGTCAAACTGATGTCACAGTAGTTTTTGTTAGCTTTAAA
TCATTTTTGCTTTAGTCTTTTTAAAGGAAAATAACAAAACTATGCTGTTTATATTGTCATTAAATTATAC
AATCAAACAAATGCCAAATGAATTGCCTAATTGCTGCAAAGTATAACCCAGATAGGAAATCATATGTTTT
TTTCCAAGAGTCATTCTAATATTTGATTATGTTATGTGTGCTTTTATGAAAGATTGTTATTTTTATATAT
CAAGATGATAGAACCTGGAATGTTAGGATTTTGAAATGTTAGACTTGGAAGGGGCCTGGTCTGTCAACTA
GTCCAACCCCTTAAAATTCATAGAGGAGCAAACTGGGGCCCATTGAAGGGTGAAGAGTTACTCAAGGTCA
AACAGCTGGTAACAGAATCAAGACTAAGACCTAATTTACCTTTCCATACTCTTTTTTTTTCTCAACTTCA
TCTATATAAAATCAGGCTTTTAAACATAACCACTAATATTTACCTGAAGATAACCATGAGTAAAGTATAC
TTTTGCATTAATTTTTTGAGCTTATATGCAAACATAATAAATATTATTAAATATCAGGAAAGCTAACATT
TCATACAAGATAGCTTCAGACCAAATTCAAATTGAATTTGAATAAATTAGAAATACTGTGCATACATAAC
CTTCTTGTGCACCATGAGTATTTGGAAAGTTAATCCTTGTTTTTGTCGTGTCTATAAAGGAAGAACAAAA
CAAAATAAAAACAGAGCCCTAGAGAAATGCTGTTACTTTTTATTTTTACACCCATCAGATTTAAGGAAAA
GACTTTTTAGCCATTATAATCTAGTGGTTGGAAGGAATGAAGAAGCTTTTTTAGTAATAGGTCCAGATAT
GAGTGCTAAAAATAAAGATGATAGCATGTTCTTCTGTCTTCCATAGTTATTACAACTATGAGAGCCTCCC
AAGTCATCTTATCAACTCAACTCCCTTTTTTTTGTCTTAATGTTGCACATAAGTTTATACAGAGTGGATG
ACCACACTAGCACAGAAGAGAACAACATGTATTAAAGCAGGTGATTCCTCCCCTTGGCGGGAGAGCTCTC
TCAGTGTGAACATGCCTTCTGTGGGCGGAAATCAGGAAGCCACCAGCTGTTAATGGAGAGTGCCTTGCTT
TTATTTCAGACAGCAGAGTTTTCCAAAGTTTCTCTGCTCCTCTAACAGCATTGCTCTTTAGTGTGTGTTA
ACCTGTGGTTTGAAAGAAATGCTCTTGTACATTAACAATGTAAATTTAAATGATTAAATTACATTTTATC
AATGGC
SEQ ID NO: 12 (NM_173199.4, NR4A3 isoform c mRNA):
GCGCAGCCGGGAGAGCGGAGTCTCCTGCCTCCCGCCCCCCACCCCTCCAGCTCCTGCTCCTCCTCCGCTC
CCCATACACAGACGCGCTCACACCCGCTCCCTCACTCGCACACACAGACACAAGCGCGCACACAGGCTCC
GCACACACACTTCGCTCTCCCGCGCGCTCACACCCCTCTTGCCCTGAGCCCTTGCCGGTGCAGCGCGGCG
CCGCAGCTGGACGCCCCTCCCGGGCTCACTTTGCAACGCTGACGGTGCCGGCAGTGGCCGTGGAGGTGGG
AACAGCGGCGGCATCCTCCCCCCTGGTCACAGCCCAAGCCAGGACGCCCGCGGAACCTCTCGGCTGTGCT
CTCCCATGAGTCGGGATCGCAGCATCCCCCACCAGCCGCTCACCGCCTCCGGGAGCCGCTGGGCTTGTAC
ACCGCAGCCCTTCCGGGACAGCAGCTGTGACTCCCCCCCAGTGCAGATTTCGGGACAGCTCTCTAGAAAC
TCGCTCTAAAGACGGAACCGCCACAGCACTCAAAGCCCACTGCGGAAGAGGGCAGCCCGGCAAGCCCGGG
CCCTGAGCCTGGACCCTTAGCGGTGCCGGGCAGCACTGCCGGCGCTTCGCCTCGCCGGACGTCCGCTCCT
CCTACACTCTCAGCCTCCGCTGGAGAGACCCCCAGCCCCACCATTCAGCGCGCAAGATACCCTCCAGATA
TGCCCTGCGTCCAAGCCCAATATAGCCCTTCCCCTCCAGGTTCCAGTTATGCGGCGCAGACATACAGCTC
GGAATACACCACGGAGATCATGAACCCCGACTACACCAAGCTGACCATGGACCTTGGCAGCACTGAGATC
ACGGCTACAGCCACCACGTCCCTGCCCAGCATCAGTACCTTCGTGGAGGGCTACTCGAGCAACTACGAAC
TCAAGCCTTCCTGCGTGTACCAAATGCAGCGGCCCTTGATCAAAGTGGAGGAGGGGGGGGCGCCCAGCTA
CCATCACCATCACCACCACCACCACCACCACCACCACCATCACCAGCAGCAGCATCAGCAGCCATCCATT
CCTCCAGCCTCCAGCCCGGAGGACGAGGTGCTGCCCAGCACCTCCATGTACTTCAAGCAGTCCCCACCGT
CCACCCCCACCACGCCGGCCTTCCCCCCGCAGGCGGGGGCGTTATGGGACGAGGCACTGCCCTCGGCGCC
CGGCTGCATCGCACCCGGCCCGCTGCTGGACCCGCCGATGAAGGCGGTCCCCACGGTGGCCGGCGCGCGC
TTCCCGCTCTTCCACTTCAAGCCCTCGCCGCCGCATCCCCCCGCGCCCAGCCCGGCCGGCGGCCACCACC
TCGGCTACGACCCGACGGCCGCTGCCGCGCTCAGCCTGCCGCTGGGAGCCGCAGCCGCCGCGGGCAGCCA
GGCCGCCGCGCTTGAGAGCCACCCGTACGGGCTGCCGCTGGCCAAGAGGGCGGCCCCGCTGGCCTTCCCG
CCTCTCGGCCTCACGCCCTCCCCTACCGCGTCCAGCCTGCTGGGCGAGAGTCCCAGCCTGCCGTCGCCGC
CCAGCAGGAGCTCGTCGTCTGGCGAGGGCACGTGTGCCGTGTGCGGGGACAACGCCGCCTGCCAGCACTA
CGGCGTGCGAACCTGCGAGGGCTGCAAGGGCTTTTTCAAGAGAACAGTGCAGAAAAATGCAAAATATGTT
TGCCTGGCAAATAAAAACTGCCCAGTAGACAAGAGACGTCGAAACCGATGTCAGTACTGTCGATTTCAGA
AGTGTCTCAGTGTTGGAATGGTAAAAGAAGTTGTCCGTACAGATAGTCTGAAAGGGAGGAGAGGTCGTCT
GCCTTCCAAACCAAAGAGCCCATTACAACAGGAACCTTCTCAGCCCTCTCCACCTTCTCCTCCAATCTGC
ATGATGAATGCCCTTGTCCGAGCTTTAACAGACTCAACACCCAGAGATCTTGATTATTCCAGAGTAAGTT
TTATGATTTCCTGCTTTCAAATGAATGATCAGGGTCTCTATTTATGGCTACTAGTAATAAGAGTTGATTG
AATGATTTTGTGTCTGGCACCATGTTAGACAGTTTTCATACTTTTTCTATATTTCTCGCTTCATTTAGCA
ATTCAGTGCATCCATTGCAGCAAATAATTTTTGCCTTATTGAATCTCTAAATGCCTTAACAAGTGACCCT
GACAGTGCTGCACCTGTCATACACATTGTTGCAGGATTCCTGGTGGTTGTGCCAATGAAAATCTGCACAG
ACAAACTACAATTTGTAGATTTATCTCGTGATCTAGACAAAGTGACTACTGTTTTTTTTCATATTGTGTT
CAAACCATCTGGGTGAGCCTCAAGTTATTACTAAGCAGTTTATCCAATTGCATCAGCATTGATTGACCTG
CTGCTTATTCAGAGGGATTAGACCATTGGTGGAGAGAAGCGAGCTCTGGCTGTGACATCAGTTGTCAGGA
GAACTGGGATGGAACGCCACCTCTGCTACTTCCTGCCTGTGTCACCTGGTTCCTGCTTTTGGCTCTCTCC
TGCCTACAGTGGAAATGGCTACAATATTTAACCTCAGAGGGGTGTTCTGAACACCAGTCACACAAGAATG
ATGGTTGTGAAAATGTTTTTAAGACCATAAATGACGATTGGTGTTGTTATAGTTAAGTCAACCAAAACAT
ATCAACATTTTTATTTCATATGGGATATAGTAAAATCCCATCAGTCTGAACCCTGCAAATTCCTCAAAAT
TTGGATAACACAGAGAAGAACCATGAGAAAGGATTTCCTGAGCAAATGAATAAACAGCTCTGTAAGGCAA
SEQ ID NO: 13 (NP_001189162.1, NR4A1 isoform 2, Homo sapiens):
MWLAKACWSIQSEMPCIQAQYGTPAPSPGPRDHLASDPLTPEFIKPTMDLASPEAAPAAPTALPSFSTFM
DGYTGEFDTFLYQLPGTVQPCSSASSSASSTSSSSATSPASASFKFEDFQVYGCYPGPLSGPVDEALSSS
GSDYYGSPCSAPSPSTPSFQPPQLSPWDGSFGHFSPSQTYEGLRAWTEQLPKASGPPQPPAFFSFSPPTG
PSPSLAQSPLKLFPSQATHQLGEGESYSMPTAFPGLAPTSPHLEGSGILDTPVTSTKARSGAPGGSEGRC
AVCGDNASCQHYGVRTCEGCKGFFKRTVQKNAKYICLANKDCPVDKRRRNRCQFCRFQKCLAVGMVKEVV
RTDSLKGRRGRLPSKPKQPPDASPANLLTSLVRAHLDSGPSTAKLDYSKFQELVLPHFGKEDAGDVQQFY
DLLSGSLEVIRKWAEKIPGFAELSPADQDLLLESAFLELFILRLAYRSKPGEGKLIFCSGLVLHRLQCAR
GFGDWIDSILAFSRSLHSLLVDVPAFACLSALVLITDRHGLQEPRRVEELQNRIASCLKEHVAAVAGEPQ
PASCLSRLLGKLPELRTLCTQGLQRIFYLKLEDLVPPPPIIDKIFMDTLPF
SEQ ID NO: 14 (NP_775292.1, NR4A1 isoform 1b, Homo sapiens):
MHDSIRFGNVDMPCVQAQYSPSPPGSSYAAQTYSSEYTTEIMNPDYTKLTMDLGSTEITATATTSLPSIS
TFVEGYSSNYELKPSCVYQMQRPLIKVEEGRAPSYHHHHHHHHHHHHHHQQQHQQPSIPPASSPEDEVLP
STSMYFKQSPPSTPTTPAFPPQAGALWDEALPSAPGCIAPGPLLDPPMKAVPTVAGARFPLFHFKPSPPH
PPAPSPAGGHHLGYDPTAAAALSLPLGAAAAAGSQAAALESHPYGLPLAKRAAPLAFPPLGLTPSPTASS
LLGESPSLPSPPSRSSSSGEGTCAVCGDNAACQHYGVRTCEGCKGFFKRTVQKNAKYVCLANKNCPVDKR
RRNRCQYCRFQKCLSVGMVKEVVRTDSLKGRRGRLPSKPKSPLQQEPSQPSPPSPPICMMNALVRALTDS
TPRDLDYSRYCPTDQAAAGTDAEHVQQFYNLLTASIDVSRSWAEKIPGFTDLPKEDQTLLIESAFLELFV
LRLSIRSNTAEDKFVFCNGLVLHRLQCLRGFGEWLDSIKDESLNLQSLNLDIQALACLSALSMITERHGL
KEPKRVEELCNKITSSLKDHQSKGQALEPTESKVLGALVELRKICTLGLQRIFYLKLEDLVSPPSIIDKL
FLDTLPF
SEQ ID NO: 15 (NP_002126.2, NR4A1 isoform 1, homo sapiens):
MPCIQAQYGTPAPSPGPRDHLASDPLTPEFIKPTMDLASPEAAPAAPTALPSFSTEMDGYTGEFDTFLYQ
LPGTVQPCSSASSSASSTSSSSATSPASASFKFEDFQVYGCYPGPLSGPVDEALSSSGSDYYGSPCSAPS
PSTPSFQPPQLSPWDGSFGHFSPSQTYEGLRAWTEQLPKASGPPQPPAFFSFSPPTGPSPSLAQSPLKLF
PSQATHQLGEGESYSMPTAFPGLAPTSPHLEGSGILDTPVTSTKARSGAPGGSEGRCAVCGDNASCQHYG
VRTCEGCKGFFKRTVQKNAKYICLANKDCPVDKRRRNRCQFCRFQKCLAVGMVKEVVRTDSLKGRRGRLP
SKPKQPPDASPANLLTSLVRAHLDSGPSTAKLDYSKFQELVLPHFGKEDAGDVQQFYDLLSGSLEVIRKW
AEKIPGFAELSPADQDLLLESAFLELFILRLAYRSKPGEGKLIFCSGLVLHRLQCARGFGDWIDSILAFS
RSLHSLLVDVPAFACLSALVLITDRHGLQEPRRVEELQNRIASCLKEHVAAVAGEPQPASCLSRLLGKLP
ELRTLCTQGLQRIFYLKLEDLVPPPPIIDKIFMDTLPF
SEQ ID NO: 16 (NP_775180.1 (SEQ ID NO: 16, NR4A1 isoform 1b, homo sapiens):
MPCIQAQYGTPAPSPGPRDHLASDPLTPEFIKPTMDLASPEAAPAAPTALPSFSTFMDGYTGEFDTFLYQ
LPGTVQPCSSASSSASSTSSSSATSPASASFKFEDFQVYGCYPGPLSGPVDEALSSSGSDYYGSPCSAPS
PSTPSFQPPQLSPWDGSFGHFSPSQTYEGLRAWTEQLPKASGPPQPPAFFSFSPPTGPSPSLAQSPLKLF
PSQATHQLGEGESYSMPTAFPGLAPTSPHLEGSGILDTPVTSTKARSGAPGGSEGRCAVCGDNASCQHYG
VRTCEGCKGFFKRTVQKNAKYICLANKDCPVDKRRRNRCQFCRFQKCLAVGMVKEVVRTDSLKGRRGRLP
SKPKQPPDASPANLLTSLVRAHLDSGPSTAKLDYSKFQELVLPHFGKEDAGDVQQFYDLLSGSLEVIRKW
AEKIPGFAELSPADQDLLLESAFLELFILRLAYRSKPGEGKLIFCSGLVLHRLQCARGFGDWIDSILAFS
RSLHSLLVDVPAFACLSALVLITDRHGLQEPRRVEELQNRIASCLKEHVAAVAGEPQPASCLSRLLGKLP
ELRTLCTQGLQRIFYLKLEDLVPPPPIIDKIFMDTLPF
SEQ ID NO: 17 (NP_001189163.1, NR4A1 isoform 3, homo sapiens):
MTSAQYKIKILIEGLHHGQRPGPAPPRQPGSFCWALKADGIMWLAKACWSIQSEMPCIQAQYGTPAPSPG
PRDHLASDPLTPEFIKPTMDLASPEAAPAAPTALPSFSTFMDGYTGEFDTFLYQLPGTVQPCSSASSSAS
STSSSSATSPASASFKFEDFQVYGCYPGPLSGPVDEALSSSGSDYYGSPCSAPSPSTPSFQPPQLSPWDG
SFGHFSPSQTYEGLRAWTEQLPKASGPPQPPAFFSFSPPTGPSPSLAQSPLKLFPSQATHQLGEGESYSM
PTAFPGLAPTSPHLEGSGILDTPVTSTKARSGAPGGSEGRCAVCGDNASCQHYGVRTCEGCKGFFKRTVQ
KNAKYICLANKDCPVDKRRRNRCQFCRFQKCLAVGMVKEVVRTDSLKGRRGRLPSKPKQPPDASPANLLT
SLVRAHLDSGPSTAKLDYSKFQELVLPHFGKEDAGDVQQFYDLLSGSLEVIRKWAEKIPGFAELSPADQD
LLLESAFLELFILRLAYRSKPGEGKLIFCSGLVLHRLQCARGFGDWIDSILAFSRSLHSLLVDVPAFACL
SALVLITDRHGLQEPRRVEELQNRIASCLKEHVAAVAGEPQPASCLSRLLGKLPELRTLCTQGLQRIFYL
KLEDLVPPPPIIDKIFMDTLPF
SEQ ID NO: 18 (NP_006177.1, NR4A2 isoform a, homo sapiens):
MPCVQAQYGSSPQGASPASQSYSYHSSGEYSSDFLTPEFVKFSMDLTNTEITATTSLPSESTEMDNYSTG
YDVKPPCLYQMPLSGQQSSIKVEDIQMHNYQQHSHLPPQSEEMMPHSGSVYYKPSSPPTPTTPGFQVQHS
PMWDDPGSLHNFHQNYVATTHMIEQRKTPVSRLSLFSFKQSPPGTPVSSCQMRFDGPLHVPMNPEPAGSH
HVVDGQTFAVPNPIRKPASMGFPGLQIGHASQLLDTQVPSPPSRGSPSNEGLCAVCGDNAACQHYGVRTC
EGCKGFFKRTVQKNAKYVCLANKNCPVDKRRRNRCQYCRFQKCLAVGMVKEVVRTDSLKGRRGRLPSKPK
SPQEPSPPSPPVSLISALVRAHVDSNPAMTSLDYSRFQANPDYQMSGDDTQHIQQFYDLLTGSMEIIRGW
AEKIPGFADLPKADQDLLFESAFLELFVLRLAYRSNPVEGKLIFCNGVVLHRLQCVRGFGEWIDSIVEFS
SNLQNMNIDISAFSCIAALAMVTERHGLKEPKRVEELQNKIVNCLKDHVTENNGGLNRPNYLSKLLGKLP
ELRTLCTQGLQRIFYLKLEDLVPPPAIIDKLFLDTLPF
SEQ ID NO: 19 (NP_775265.1 (SEQ ID NO: 19, NR4A2 isoform d, homo sapiens):
MDNYSTGYDVKPPCLYQMPLSGQQSSIKVEDIQMHNYQQHSHLPPQSEEMMPHSGSVYYKPSSPPTPTTP
GFQVQHSPMWDDPGSLHNFHQNYVATTHMIEQRKTPVSRLSLFSFKQSPPGTPVSSCQMREDGPLHVPMN
PEPAGSHHVVDGQTFAVPNPIRKPASMGFPGLQIGHASQLLDTQVPSPPSRGSPSNEGLCAVCGDNAACQ
HYGVRTCEGCKGFFKRTVQKNAKYVCLANKNCPVDKRRRNRCQYCRFQKCLAVGMVKEVVRTDSLKGRRG
RLPSKPKSPQEPSPPSPPVSLISALVRAHVDSNPAMTSLDYSRFQANPDYQMSGDDTQHIQQFYDLLTGS
MEIIRGWAEKIPGFADLPKADQDLLFESAFLELFVLRLAYRSNPVEGKLIFCNGVVLHRLQCVRGFGEWI
DSIVEFSSNLQNMNIDISAFSCIAALAMVTERHGLKEPKRVEELQNKIVNCLKDHVTENNGGLNRPNYLS
KLLGKLPELRTLCTQGLQRIFYLKLEDLVPPPAIIDKLFLDTLPF
SEQ ID NO: 20 (NP_008912.2 (SEQ ID NO: 20, NR4A3 isoform a, homo sapiens):
MPCVQAQYSPSPPGSSYAAQTYSSEYTTEIMNPDYTKLTMDLGSTEITATATTSLPSISTFVEGYSSNYE
LKPSCVYQMQRPLIKVEEGRAPSYHHHHHHHHHHHHHHQQQHQQPSIPPASSPEDEVLPSTSMYFKQSPP
STPTTPAFPPQAGALWDEALPSAPGCIAPGPLLDPPMKAVPTVAGARFPLFHFKPSPPHPPAPSPAGGHH
LGYDPTAAAALSLPLGAAAAAGSQAAALESHPYGLPLAKRAAPLAFPPLGLTPSPTASSLLGESPSLPSP
PSRSSSSGEGTCAVCGDNAACQHYGVRTCEGCKGFFKRTVQKNAKYVCLANKNCPVDKRRRNRCQYCRFQ
KCLSVGMVKEVVRTDSLKGRRGRLPSKPKSPLQQEPSQPSPPSPPICMMNALVRALTDSTPRDLDYSRYC
PTDQAAAGTDAEHVQQFYNLLTASIDVSRSWAEKIPGFTDLPKEDQTLLIESAFLELFVLRLSIRSNTAE
DKFVFCNGLVLHRLQCLRGFGEWLDSIKDFSLNLQSLNLDIQALACLSALSMITERHGLKEPKRVEELCN
KITSSLKDHQSKGQALEPTESKVLGALVELRKICTLGLQRIFYLKLEDLVSPPSIIDKLFLDTLPF
SEQ ID NO: 21 (NP_775292.1, NR4A3 isoform b, homo sapiens):
MHDSIRFGNVDMPCVQAQYSPSPPGSSYAAQTYSSEYTTEIMNPDYTKLTMDLGSTEITATATTSLPSIS
TEVEGYSSNYELKPSCVYQMQRPLIKVEEGRAPSYHHHHHHHHHHHHHHQQQHQQPSIPPASSPEDEVLP
STSMYFKQSPPSTPTTPAFPPQAGALWDEALPSAPGCIAPGPLLDPPMKAVPTVAGARFPLFHFKPSPPH
PPAPSPAGGHHLGYDPTAAAALSLPLGAAAAAGSQAAALESHPYGLPLAKRAAPLAFPPLGLTPSPTASS
LLGESPSLPSPPSRSSSSGEGTCAVCGDNAACQHYGVRTCEGCKGFFKRTVQKNAKYVCLANKNCPVDKR
RRNRCQYCRFQKCLSVGMVKEVVRTDSLKGRRGRLPSKPKSPLQQEPSQPSPPSPPICMMNALVRALTDS
TPRDLDYSRYCPTDQAAAGTDAEHVQQFYNLLTASIDVSRSWAEKIPGFTDLPKEDQTLLIESAFLELFV
LRLSIRSNTAEDKFVFCNGLVLHRLQCLRGFGEWLDSIKDESLNLQSLNLDIQALACLSALSMITERHGL
KEPKRVEELCNKITSSLKDHQSKGQALEPTESKVLGALVELRKICTLGLQRIFYLKLEDLVSPPSIIDKL
FLDTLPF
SEQ ID NO: 22 (NP_775291.1 (SEQ ID NO: 22, NR4A3 isoform c, homo sapiens):
MPCVQAQYSPSPPGSSYAAQTYSSEYTTEIMNPDYTKLTMDLGSTEITATATTSLPSISTFVEGYSSNYE
LKPSCVYQMQRPLIKVEEGRAPSYHHHHHHHHHHHHHHQQQHQQPSIPPASSPEDEVLPSTSMYFKQSPP
STPTTPAFPPQAGALWDEALPSAPGCIAPGPLLDPPMKAVPTVAGARFPLFHFKPSPPHPPAPSPAGGHH
LGYDPTAAAALSLPLGAAAAAGSQAAALESHPYGLPLAKRAAPLAFPPLGLTPSPTASSLLGESPSLPSP
PSRSSSSGEGTCAVCGDNAACQHYGVRTCEGCKGFFKRTVQKNAKYVCLANKNCPVDKRRRNRCQYCRFQ
KCLSVGMVKEVVRTDSLKGRRGRLPSKPKSPLQQEPSQPSPPSPPICMMNALVRALTDSTPRDLDYSRVS
FMISCFQMNDQGLYLWLLVIRVD
SEQ ID NO: 23 (NC_000020.11: 32358062-32439319 Homo sapiens chromosome 20,
GRCh38.p13 Primary Assembly, AXSL1 genomic sequence):
GCACCTCGCGAGACTCCACCCAGCCCCTTCCCCGAACAGCGACCCGTCCCCGTTCCTAGCTGAGGTCGGT
GCTAGGCGGAGGACGCGAGAGGGAGAGCGGAAGGGGGCCGCCGGGCACGCCGGGAAGTGTGGTCCACGCG
CTTCACGCGGCCATATTCTTCCGGAGCGGGGGAGGCCAGAGAGGCCGTTGGGACTACGACTCCCAGGCGC
CCTTTCGGTTTGTTTTTGTTCAAAAAAAAAAAACCCCACACTCCCCCTCCTCACTCTTCACACACACTCA
CACACACCCACGGCAGACACGCACGCACCCGGGCGCCGAAGGGAAAGCCGCGTCTCGCCCTCCCGCCCCG
CCGTCGGTCCTGTCTCAGTCCCTCAGCAGAGCGGGAAAGCGGAGGCCGGAGCCGTGACCTCTGACCCCGT
GGTTATGCGGAGCCGCCGCATTCCTTAGCGATCGCGGGGCAGCCGCCGCTGCCGCCGTGGGCGACTGACG
CAGCGCGGGCGCGTGGAGCCGCCGCCGCCCCTCCCCCACCGCCGCTCTCGCGCCAGCCGGTCCCCGCGTG
CCCGCCCCTTCTCCCCGGCCGCACCCGAGACCTCGCGCGCCGCCGCTGCCACGCGCCCCCCCCACCGCCG
CCGCCGCCCCAGCCCCGCGCCACCGCCCCAGCCCGCCCAGCCCGGAGGTCCCGCGTGGAGCTGCCGCCGC
CGCCGGGGAGAAGGATGAAGGACAAACAGAAGAAGAAGAAGGAGCGCACGTGGGCCGAGGCCGCGCGCCT
GGTGAGGCGGACAGCCGAGGGGGGCTCCGTGGGGCCCGGGGTGGGGGGGGCTCGCCGCGCACCCCCCCAC
TGGGGGGGGAGGGGCAAGGCCCTGCCCACCGCGGGAGGGGGCGGGGCCATCTTCCTTTAAGAACGGGACA
GCCCCGCAAGGCGAGGGGTGGGGAGCGCTCCGCCGGGATGGGATGTGGCGCCTTTTTCCGCTCGCCCTCG
CGCCCCCCCCGCCCCGCGCAGCTAAATTCCGGCGGAGGGGCGAGCTGGCAGGCCGGCTCCTCCCACTCTG
GGCAGCGGGGTCCCGCGTCCCCTCCCCCACTATTTGGCAGCGTCTGGGGGTCTGGGGCAGCTTCGTTCAT
TCACCCGGGGGAGTTGGGTTTCCGGGAAGGGTCGGAAGCTCCTCCCTCGCTTCCTGGTGGGTAATGGGGT
GGTGCCTTTGACTCCGGGGGTGGAAAAGCGACCCCACATTCAAGGACGCCAATGGCATGTTGAGCTTTCC
CAATCTAAACCAGGTGCGTGGAGGGAAGCAAGTGCTTACTCCCAGCTTGAACCCTGAGCAGCGGTTCTCT
AACTTTAGTGTCCTGTGGGCGACACCTGGGAGGCGGTTAGGAACGCTACTCCTAGGGCCCTTCGTAAGAC
CTGCTGAGCCTCAGGGTCTGGGATAGAGCCTGGGAATCTGCGCTTTCACCAGCTTCCCAGGTGATTCAAA
CGCAGTGGCTCACGGACCCCACTTTGAGAAACGCAGACTGGGCTGTTTGCCTTCAGAAGCTGGACTGGAA
ATTGGTATTTTGAGGAGAACTGAATCCACTTTGCAAAGAATGTATGTTGGACAAAGACAAAGTATCTCTG
TTGGGAGACGTTGTTCACTGAGGATTTAGCAAGGGAGAGCGTAACTTGAGTGTTTGAAGCCGTCTCGTTC
AACCGATGGGGGTTGTGAATTTTGTGTTCAGAGCGTCAGAGGACTTGCAGGTGAAATAGCTTCCTTTTGG
ATTACTCCCACAAAGCTCAGACACTTTGAGACATATTCGGGTAATTATCTGCTGATACCAGTAGTAACAG
GTTATACTAAGATGTCAGTTCCTAAGATCTGTCACACAGCGAGGTTGTGGTGCGGATAGCATAAGTGTCT
CTTGACACTGTAGTTTATATGTAATGGTTGTAATTCAGTTTTAATGATGAGTGAAGCTCAGACTTAATTT
TCTCTCTTATTTTGAAGTGAGTTTTCTGCTTCAAAGGTGTTGTGCAAAAAAAAAAAAAATAGACTGTATT
GGGGGGGTTGACACAAACCTGAAGTACACCTTTTTCTTTCTCGAAGTCTGTAAAAGTTACAGATTTATAA
AAACTGAAGGAATTTGGGCTACAGAATTTTCATTGGTGCTTGCTATCCTCTTCCCTAGTCATTTGTTGCC
CAGGGAACAAAAATAGCAATTGTTTGTATGCTGAAGTTTTGTGCTTCAGTTTATTTTGGCCTGGCATGTG
AAGTTTGAAGATTTTGAGGTTCCGAAATATTTTTTTTCTGATTGAGTCAAAATTGGAAGCTTGGGCTAGT
TTTCAGGATCTTGTAGAAATTTCAGGATTTCTCTAACGGTTCTGCACCTGGAAACAAATATAGGCCTGAT
GGGGAGTGGGAGAGAGCTCTCGTTCTCCTCCTCGACAATGAGTTTTGTGTGTTCATTGACATTCTCCTTG
GCGTTCCTGTTATGCGGGTGTTTATGACCCTGGGCACCCAAGGAAATTTGCTTGCCAGAGTTTTACTTGT
TAGAGGTAGGTGAAAGTACTTTTAAGTCTTGTGTTATTTATGTCTTGACAGAATTATGAAATCTGTTTGT
CTCAAAGTTCTGTTGGCTCTTACTAGTTGACTTTAGGAATTTGGGGTCCACTGTTAAGTCTCTTTAGATT
CTCGAATTTACCGTGGTCTGAACTTCAACATCGAGGTTGCTATTTCTGTTTTATCATTGACTCAACTTCA
AGCCAGGTTATCAGGGTAAGTTATTCCTCTTTTTCTTCTAAGGCTCTTCATTAAAAATAGTAATATGAAT
AGGAATTACTTTGTTTGTTTGTTTTTCGTTTGTTTGCCACCACACCCGGCTAATTTTTTATTTTTAGTAA
AGATGGGGTTTTTCCATGCTGGTCAGGCTGGTCTCGAACTCGTGACCTTAGGTTATCCGCCCACCTTGGC
CTCCCAAAGTGCTGGGATTACAGGCGTGAGCCACCGCGCCCAGCCTATGAGTAGGAATTACTTTGTGGTG
CTGTTATACATGTGAAAAAGGATTTGCGAGACTTTGGGAGTCTGAGGTGGAATAATCACTTGAGGCTAGG
AATTCGAGATGAGCCTGGGCAACATAGACCCGGTCTCTACAACAACAGCAACAAAATGAGCTGGGCATGG
TGGTACACACCTGTGGTCCTAGCTACTCAGGAGGCTGAGGATAGCTTAGGCCGAGGAGTTGGAGGCTGCA
GTGCCCAATGATTGATTGTACCACTGCACTCCAGCCTGGGTGATAGAGGGAGACCCTGACTCTAAAAAAA
TAATGAGGCTGGGCGCTGTGGCTCACGCCTGTAATCCCAGCACTTTGGGACGCCGAGGTGGGTGGATCAC
AGGGTCAGGAGTTCTAGACCAGCTTGGCCAACAAGGTAAAACCCAGTCTCTACTAAAAATACAAAAATTA
GCCGGGTGTGGTGGTGCACGCCTGTAATCCCAGCTACTGAGGGGGCTGAGACAGGAGAATCACTTGAACT
TGGGAGGTGGAGGTTGCAGAGAGCCAAGGCCATGCCACTGTCCAGCCTGGGTGACAGAGCAAGACTCGTC
TCAAAAAAAAAAAAAAAAGAAAAAAAGAAAAAGGACTTGTTGAGAATGTGATATGCAATTTGAGTATCTT
GTGTGTGTTTTTTGTTTTAGAATTTTCCATTTCTTTGAATGGGACTGGATGGTAGGTGAGGGGGAACTTT
TTAAGAGTCCCAGGCCGGGCACGGTGGCTCACGCCTGTAATCCCAGCTCTTTGGGAGGCCGAGGCAGACG
GATCACCTGAGGCCAGAAGTTCGAGACCAGCCTGGCCAACATGGTGAAACCCCGTCTCTACTAAAAATAC
AAAAATTAGCCAGGCATGGTGATGGTGCATGCCTGTAACCTCAGCTACTCGGGAGGCTGAGGCAGGAGAA
TCACTTGAACTCGGGAGGTGGAGGTTGCAGTGAACCCAGATCACACCACTGCACTCCAGCGGCGACAGTG
AGACTCTGTCTCAAAAACAAAACAAAAGAAAAAACCAGTCCTCTTAATGTTTAAAAAAAGTTGCTGTAAC
ACTGTACTGGAGAATTTGTGAGAGCGTACACTGGACTGAGAGAAACTTGACTAGGAGGGAACGTAGGACA
GTTGATTAGAACACCTGAACACTTCTCTTTGGGCAGGTGAATTTGGAGTGCTTGCAATGGCAATTGTATG
CACTTCCATGGGAACGCTTTCAGATTGCTCCTGACCCCACTCAGATTGGTGACTAGAGTGTTACTGGGAA
TCCTTTTAAAAATTGTTTTTGTAGGCTGTGCGCGGTGGCTCACGCCTGTAATCCCAGCACTTTGGGAGGC
CGAGGCGGGCGGATCACGAGGTCAGGAGATCGAGACCATCCTGGCTAACACGGTGAAACCCCATCTCTAC
TAAAAATACAAAAAATTAGCCAGGCGTGGTGGCGAGTGCCTGTAGTCCCAGCTACTCGGGAGGCTGAGGC
CGGAGAATGGCGTGAACGCGGGAGGCGGAGCTTGCAGTGAGCCGAGATCGAGCTACTGCACTCCATCCAG
CCTGGGCGACAGAGCAAGACTCTGTCTCAAAAACAAACAAACAAAACAACAACAACAAAAAAACCTTTCG
TAGAGGAAAGAAGATTCTTAAGAATTTCAGTATAGTGTACCCCAGGAGATTGATTTTTGCAGATGTTAAT
TATGCTGTTGAAAAATAGTCTTTCTTTCACACATTTTGTAGTATTGTCTTTTCTAGGTGTAAGAAGTAGA
CCTTATGTGGTTGAGTGGATATAAATCCTCCAACTTGTTATAAACAGTTTGTTTGGGAGAAATAGCCAAG
GGGATTTCCCTTTAAATCGAGGACGCTAAATGGATTCTCCCTGGCTTCGAGAAACTCTTTGTTGTATGCT
TTATCTTTTACTTTGATTCATGACTGACCAGTTCTTACATCAAACATTTTTTTCCCCTTTGAAGTAAGAG
AAGAGATACTTAAAAAAAAAAATGTAGTCTAGAAAGAGACTAGGAATAGAACACTGCTGACTTGTTCTTA
TACCAAGAGCTGGAGGCATAAACAGTTTTAATAAAAAAGAATACTTTGTATCTTTTGATGTAACTTCCCT
TTAGAGATTCTGTTTCCCCTGTCTTAAATTCTCAGGCTATTTCTGATTGCTGTTTAAATAATTGGTAGTT
ACTATTTGAGTCATGCTTCTTAAAAAAAAAAGATATAAAAATTCGCCAGCTCAGCTTTCATTTAATTAGT
ATCAAAGAAATGTTATCTGTGGCAGTGAACATTAGTAGCTTGTTCCAGGGTTTATTGTAGGTTTGTTCAA
GTCAGTACTGCCCATCTTGGCACTTAGGTTCTTGGAATACAGAGGTGGATAAGGTTTGCCCCTCAGGGCT
TATGGTCTTCTGGGGATGAAGAAGGTCCAGACGCAGGAACAGAATTTTACTACAGTCATGATACGTGTTA
TCATAGTTGTATAAATCATAAACAGATATCCCAGTTGCTGCCTCTTGTCTAGTCATAGTTTCTCCTTTCA
CTCTCAACTGTCCTGGTTTGGAAGATAATTGTATGTGTTATGAAACAATGGGAACATTGAAAAGAGACAT
TTACCTCTGCCTGGCAGAGATCAGGGAAGGCTTCCTGGAGGAAGGTGCTTTCTGAGCTGAATCTTGAAGG
GTAACTAGGAGTTAGGCCAATCACAGAGGGCATAAGCAGGAGCTGGTGACAACACATAGCAGGAGAGAAA
GAGTCAGAGTGGGTGAGAGGGAGTGTGTGTATTTGCAACGTGTGGGAATTAGGTATATCTACAAATTATT
GGAACATAAAGTTTGTAGAGTGTCACTGTGGATGGGTAGAGAGGCAGAGACCAGATTGTGAGGGTTCCAT
ATGCCAGGCCAGTGAGCACCTACTCTGTGCCAGACATTGTAGGCATTACTTACATTGTCTCTAATCTGTC
CAACAACTCGAGAGGAAGACATCAGTATCCTTCAAAAGAGAACCTAGGTTCAGGTTGGGTTGTAATTGCC
CCAGTTTAGAATGTAAAAATTTGAATCTAACTCTAGATCCTGTTCTTTACCGGTGGGCCAGTGGACTTTG
GAAGCTTCAGCTTTTGCAGTATGTGATTAAGGAGCTGTTGAAGGATTTTATTTGCTTTCTATTATTATTA
TTACTATTTTTTGAGACAGGGTCTCACTCTGTCACCCTGGCTGAAGTGCAGTGGTGCCATCACAGCTCAC
TGCAGCTTCCACTTCCTGGGCTCAGATGATTCTCCCACCTCAGCCTCCTGTGTAGCTGGGATTACGGGCA
TGAGCCACCATGCCCATCTAGTTTTTTGTATTTTTTTGTAGAGACAGGGTTTCACCATGTCGCCCAGGCT
GGTTTTGAACTCCTAGGCTCAAGCAATCTGCCCACCTTGGCCTCCCAAAGTGCTGGGATTACAGGTGTGA
CCCACTTTACCCAGCCATATATATTTTTTAAATTCTTTAAAGAGATGAGGTTTCACTGTGTTACCCAGGC
TGGAGTGCAGTGGCTATACACAGGCACGATCATAGTGCACTAGAGCCTGGAGCTCCTGAGCGCAGCAAGT
GACCCTCCTGCCTCAGTCTCAGCTGAGACTATAGGTGTGCACCACCGTGCCTGGCCTGTTGAAGGATTTT
AGAGCTGAAAGTGATGAGGGCAGGTTTGCTTTGCTGGGTTGACCTCAGTCTATAACATGTAAGTTCTACC
AGAGCAGGGATTTGGTCTTGTTTACTGTGAATCCCTAGAATTGTGTCTGGCACAAGGTACGTGTTCCATG
TGTTTGTTGAATGAAGGATGACAACAGAGTCAGAATGGATTTGAGGAAAGCAGGACTGGATGGAGGGATA
AATATTAAGAGGCTGTTGTGTTGGGTCAGGTAAGAGACTGGTTTTGGGACTTGGCGGACAGATGAGCGAG
AAAGATGACACCTGCGTTTCTGAATTAGGTGATTATACAGGCTGTCAATCAAGGTAGGGAATACAGGAAG
AAGAGTGGGTCTGGAGAAGAGGTGAATTAGAGTTGCCAATTAGATGAATTAAGAGTAGAGCTGAATTAGA
GTAGATAATGAAGGGGTATAGTCAGTACACAACTGAATATTTGAATCTGAAATTTGGGGGAGAAGTCAGA
GCTAGAGGTAGAGATTAGGTAGTTATCAGAATTTAGGTAGTAGCTGAAACTGAGATGATAATGTTACCCA
TGGAGAATGGAAAATCTGAAAAGAGAAGCAAAGAATAGGACCATGGGAAATAGGGGTTTTATTTTAGTAC
CTTCTCCAAACCTTGACTTTGGTGCTGAGGTCTTATGTTCTTCCATAGAGAGTACCTTCCGCAGCAGACC
TATCAGACGAGATCGGGCACATTCAGGGTGGTATGGCTGTAGACAGCAGCATCGTATCAGACTTCTTTAT
GTTTTGCTTGTGACAGCAGTACTTGGCTATGGCAGTTAAATGGACTGTAAGATGTTAGCTTGATTTGGAG
TACCCTGAGTAGCTGCACAGGTAGACAAAGTACAAAGATATTGACAGCTTTTTTCTTCCTTTAGAAGAAT
AACATATCTAAAACAACTCTTAAAGCAAGAGAGCTGCAGTTGCACACTAAGAAAATACTGCAATACTGCC
ATGACTTGGTTATAGGTGATGCTAACATAAATGGGATATTCTGTTTCTCATTGCACCAGTCATGCAGAAT
CATAGCATCATGTGCCAGATGGCAGAACGTACTGTGGCAGTTAACACCACAAATTAACAACACAGTGGCA
GAAAGCACCGTTTAAAAATATGGGTACTTGGCCAGGCGTGGTGGCTCACTTCTATAATCCCAGCACTTTA
GGAGGCTGAGGCGGGCAGATCACCTGAGGTCAGGAGTTCGAGACTAGCTCTAGTCTCTACTAAAATACAA
AAATTAGTGTGTGTGTGTATATATATATACTAATCAAACTTCCTTGGGAAAGAAAATGTGAATGCATTTC
TGTGTCATACATTTCTTGTGCAGCACTATATATTCTGTGGTTTAGCAGTATTAACAGCATCAAGCTGAAC
AGTAACCAGTGTTGCTAAGGTCTCGCATACTATACCATGTATGCTGAGAAGATATGCATCCTTAGAATTG
AGAATTAGAACATTTCAGTTAATTGCAGTAACTTTTCAGTAACTAGATATATGTGTAATTTGATTTCTGA
AGTAATTTTAACTGTGGAAAAACTAAGTTGTCACCAGCGGTACCTCATAGCATAACTTTAGCCCATGATA
TATGGATGGATGGATATAAATGGAAATTGCACACTGAAATTAGGACGTTTATATTTCTTCAGGTATTAGA
AAACTACTCGGATGCTCCAATGACACCAAAACAGATTCTGCAGGTCATAGAGGCAGAAGGACTAAAGGAA
ATGAGGTTTGTATTGTTCTTGTTGCTTAACATGAGGGTTTCATAGGATATGTGTCTTTCTGTAGTTGTAG
TGATATGAGTAAGTACACCTGTGTATGTATTTGTGCTTCTGTGTCTGGTAGTGAGATGGAACTATCCATT
TCCCAAATTGTGTGTCGTCTCAGTGGAGGGTGGCAGTGGGTGAACATCCATTTGTTTTCATTTAGAGGTG
CAGTGGCTTTCCCCATCTCCTTGTTCTTCCTTAAATCTTGGCATAGAGCTTGTTTTTTGTGTAAGGAGTC
AGGGCACATTTTCTCTTGTGCTGTTTTGGAAGTTTCAGACCTGATAGCCTCTATTGTGCCTAACGCTACC
CTTTTCTCTATGCCGTATCACTGAAGTTCTTTCTTTTAGAATAAAAGAACAGTGGTCTCATCAACATAAA
AAACCACTGACTTTTTTCCCCTTACTAATTGCAGTCATATATGATGTGAATTTACTAAATTTTGAGGTGA
CCTTTATTTTCCAAATTTATGGTGTAGTTTTCCTGGAGAAGTAACAAGTTAGAAACATAGAATTTGCAGG
CTTGAAAGCCAGGACTATCTTTCATCTTTCTTTTTCCTCATCCTAACTTCAAACTTAAAAAAAAAAAAAA
AAAAAGCTGGGGGTAGTGGCCCATGCCTGTAATCCCAGCACTTTGGGAGGCCAAGGCAGGCAGATCACTT
AGGTCAGGAGTTCGAGACCAGCCTGGCCAACATGGCAAAACCCCGTCTCTACTAACAATACAAAGATTAG
CCGGCATGGTGACGCATGCCTGTAGCCTGTAGTTCCAGCTTGGGAGGCGGAGGCTGGAGAATCGCTTGAA
CTCAGGAGACGGAGGTTGCAGTGAGCCGAGATTGCACCACTGCACTCCAGCCTGGACAACAGAATAAGAC
TGCATCTCAAAAAACAAAACAAAACAAAAAACAAAGAAAGTACTTAAATGAGGAAAGAGGAGAGCACTAC
ACAGTCCTTGCCTGGGACTCAGAGCAAACTTCATGTTTTTGCTAGTCAAACTTAGCTGCTTTAATTGACC
TTTTAGGGTCAAAAGGCCTTCACTTTCCAGTTGTCAGTTCAAACGTTCTCAAGTTGATGTTTTGTGGGGA
GGGAAGATTGAGATTGTCTACATCACATTTATTTGACTTGTACTTATCCACTGTGTAGTGTTATAATTGT
GCTTTATTTTTGTTTTTATGGGCTATTTCCCATCATGCTGCTGTCTGCACTGCTGTTGGACCACTTTGCC
TATAGAAGGTAAATGAGTCCCTTTGGGACATATGGAATTGAGAACCTTAACTTGTTTCTGCTTCTTGTTC
TTAGAGGAATTATGAAGCAGTCATGATGGCTCATGATGAGTGAGCAGCTCTGTAATTTGTAGACAGAGGT
ATAATATGTAGGAGTCACAGAAGAGCTGTGCCAGCATGCTGTTCCCCTTGTAGCAGAGATGCTAGCACAC
CCACTTGATACTCCAGATTTTGCATAGGGTTTCTTTTAGAGATTGGTTCTTGGTGAAAAATTTTTTAGCA
TCCAGTGTGTTCCAAGGCAAAATAAGTAATAGTTTTTTAATGGTTTTTTTAGACTCAGAATTTATTTTTA
ACACTGCATAAATTAGGGCTTCTAGTTCTGAAAAATGCAAAGAGGCTTACTTAAGCTATTTATATATTTT
TTAACATGCTGGGGAGAGGGAAGAGAGGGTGCACAGGTGACTCCTAGCACTAATATGCTAGTGCCAAACT
CAGTTAATATTAATAATGGTCATTTGTTGATATTTTTGGTAGCTTTTCTATTTAAAGATTCAGTAAGTAG
CATCCTTTGCACTGAATTGTCACGTTTTGTAACCTGCTGTTGTGGCAGGGACCATGAAACAACTTAGTAT
GCCATTGTTTCAGTTTATTTTTTAAGCATTACTGGGAACTAAATCCACTTGACTTACTTTTTGCATTTAG
GTTTAAATAATTGTTCCTGGGTAGATACAGAATAAAACTCAATCTCCCCACTTCCAGCAGAGAAAAAACA
AATGAAATACTGTTCTTTTTGTGTACTTACTAGAAAATGTTTAACTGGAAAAAAAGTTCTGCTTGTTTTA
TTGGCACAGCACCATTGATTCTGTGATTCTTTGTCATAGTATTCTGTGGCAGTTTCTGAGTATGATATGT
AGATTGAAATATAGAGGCTAGGAAACTTTTAATAGTAAAATTTTACTAAAAATTTATTAACTTATTAAAA
TATAACTTGAAAAAATTGTGAGGTTTTTCTAAATTTCTTGGAAATTTGATTTATATTGTCAAAAAGCACT
GAAGAAGTGTTAGTTATGGATTTCGGGTATCACATAATGTTTATTCCAGAGTTTTCTGAGTGTGATTTAT
GTGAATTTCATTTGGTTATTCTTTTACTATTTTCACAGTGAAATTGTCATGAGATTTTTTCTTCTGTATT
TCTTGCTTAGCTTCTTCTCATTTAAGAATGAGTTGGAGGGATTAGGTCTTTAAGTAGGCAGATGGATTGT
ATAACCCTCATCCATTCTTTTGTGGTTTTACAGTGGGACTTCCCCTCTCGCATGCCTCAATGCTATGCTA
CATTCCAATTCAAGAGGAGGAGAGGGGTTGTTTTATAAACTGCCTGGCCGAATCAGCCTTTTCACGCTCA
AGGTAAGTGATATGAACTCTCTTTTGGTGCAGTGATTCTTGGACTTTTAATGTGCATAAAAATCACCTGG
TAGGTCTGGAATAGGGGCCATGAATTTGCATTTCTCATATGCAGTCAGGTGACACTGATGTTTTTCTTTT
TTTTAGACATAGTCTCGCTGTGTCACCCAGGCTGGAGTGCAATGGGGCGAGATCTCGGCTCACTGCAATC
TCTGCCTCCCATGTTCAAGTGATTCTCCCACCTCAACCTCCCAAGTAGCTGGGACTACAGGCACATGCCA
CCATGCTCGGCTAATTTTTTTATTTTTTTTAATAGAGACGGGGTTTCACCATGTTGGCCAGGCTGGTCTC
GAAATCCTGACCTCAGGTGATCCACCTACCTCGGCCTCCCAAAGTGCTGGGATTACAGGCATGAGCCACC
GCACCTGGCCAGATGCTGTTGGTTCTAAATGGCACTTTGAGGTGTTCTAATATGAATCAGATATTAGAAA
TTATTTAGTCATAGCAGCTCTTGTTTGTGTGTTCATCTTTTCTTGTCTATATCTTTGTAGATTGTACAAA
ACTGAAGCATAACTATGGGGATAATTGCAGATGTGCCTTTTTTTTTTTTTTTTTTTTTTTATTCTGAGAT
GGAGTCTCACTCTGTTGCCCAGGCTGGAGTGCAGTGGTGCGATCTCAGCTCATTGCACCCTCCACCTCCC
AGGTTCAAGCAATTCTTCTGCCTCAGCCTCCTGAGTAGCTAAGAGTGCACTACCATGCCCAGGTAATTTT
TGTATTTTTAGTAGAGACGGGGTTTCACCATGTTGGCCAGGCTCGTCTTGAACTCCTGACCTTGTGATCC
GTCCGCCTTAGCCTCCCAAAGTGCTGGGATTACAGGCGTGAGCCACTGCACCTGGCCACAGATGTGCCTT
AATTAGGATTTTGTTGTAGTAATGATGTGAAGGATAAATTACTGGTCAGCTAATGTAAGACCTACTCTAT
GGAAAATAACTGAAATGTGAACATATTTGTGGAAATTAAGTGACAGTAATTATAAAGTAAGAGTACTTAA
ATCAGGTAGAGTTCATAATAAAGCTTAAATAGGGCCGAAATGAACAGGCTAATTATTTTTCCACCATTTG
TGATATATATGGTAATCATTTAGACCGGGGGTCTGCAAAGTTTTTCTGTAAAGGACCAGAAAATCAATGT
TTTTTTGTTTTGTGGATCATACTGTCTTTGTCTTAACCACTCAGGTCTGCCATTGTAGTAGGAAAGCAGC
CATACCCAAATGAATGGGCATGGCTGTGTTCCAAAAAAAAAAAAAATTTATTTACAAATGTTTGTTTTAC
TTACGAAAACAGGCTGCAGGCCAGATTTGATTTGGGGCTGTACTTTGTCGACCCCTGATTTAGACATAAG
ATGACTTGTCGACATTGGAGAAACTGAAAATTTGATGCTGACCCTTTCGTTCTTCCATAGTGGGTTATAA
ATTTTAGCAGTCAGTACAAACTTTTTCCTTTAAGTTTTGTTTTCTTGATGGACTGTATTCAAAATAAATA
CCTGAACAAATACCAACTATATAACATGAAGCCATAAAACTTGGTCTTTATATATAAATAAAGTTTATCT
TGGGGTCAGATGTCTGTGCTGTTTTCTATATTTTTTTATATCCAATAGTTTGGTCTTTTTCCTTTCAGTT
TTGTATGCTTTTCTTGTAATCGAGCCTTGGAAGAGACAGAGCCACTGAGCCCAAAACCTAGCTTTGTGGT
CGGCTGTGGTGGCTCATGCCTGTAATCCCAGCACTTTGGGAGGCTAAGGCGGGAGGATTGCTTAAACTCA
GGAGTATGAGACCTTGTCTCTATTAAAAAAAAAAAAAAAAAAAAAAAGCCTTGCGTGGTGGTGTGTGTGC
TTATAGTCTTAGCTACTCAGGAGGCTGAAGCAGGAGGGTAGCTTGGAGATGGAGGCTGCAGTGAGCTGTG
ATCACACCACTGCTCTCCAGCCTGGGCCACATAGCAAGACCCTGTCTCAAAACAAAAACTTAGCTTTGTA
ACCTGGGAGGGATTTAACCCTTCTTTTTGGTGGAATATGCATCCCTGGCATTGAGTCAGTCCTTAATAAA
TGATAGCCTTTATGATGATCTGAGGTTTATCATTCTTAATTGCCTGTTAATGGCATTTTTCTATTAATAT
TATCTATTTTTTGAGATAGGGTCTTGTTCTGTCACCTGGGCTGGAGTGCAGTATCATAGTCACAGCTCAC
TGCAGCCTTGACCTACTAGGCTCAAGTGATCCTCCCACCTCAGCCTTCTGAGTAGCTAGGACTTCAGGCG
CATACCACTATGCCTGGCTAATTTTTTGTATTTTTTGTGGAGAAGGAGTTTTGCCATGTTGCCCAGGCTG
GTCTCGAGCTCCTGGGGTCAAGCGATCCACTGGCTTTGGCCTCCCAAACTGCCAGGATTACAGACGTGGG
CCACTGCACCTGGCCTATTATTTTTTAATTTAATATTAATTTATATTTTTAGAAACAGGGTCTCACACTG
TTGTCCACAGCTGGAGTGCAGTAGTATAATCATAGCTCACCGTAGCCTTGAACTCCTGGGCTCAAGCAGT
AGTCCCACCTCAGCCTCCCGAGTAGCGTACACCATCACCCCAGCTAATTTTTAAATTTTTTCCTAGAGAT
GAAGTCTTGCTATGTTGGTTAAACTGGTCTCAAACTCCTGGCCTCAAGTGATCTTCCCACCACAGCTTCA
CAAAGCACTGGGATGGATTACAGGTGTGAGCAACCATACCTGGCCCTCTATTATCTCATATATAGCTTAT
TGTTACATATGCTGTAATTATATATAGTTATAATTATTTAATACATATATGTTTATTACAAATATGCCTG
TTAATGGTATATTCTGTTTTTAAAAGGAAGGATAATTACTTTTTTCTATGAGTACCTTAAGGGCAGGAAC
TGAGTTTTATTTGTGTTTGTAATTTGGCTCTGGGCAGAATTCTTGGCATAGTACGTGCTTTATGTGTGAA
CTGAATTATATTTCTTCATCTTAATTTTACAGGTGTGAGCCACTGCACCAGGCCCCTTCATCTTAATTTT
AATATATCTTTGAATAAACACCATTGTATGAACCTGCTGTAAGCTTGGGAGTGGTCTGTTAGTCTACAGC
TTGTGTCTGAGATGTGCTAATTGAATATTTGCTCAGTACCTCATCTTAACTGCCTTTGGCTTTATGTTGC
TTATCCTTCATAGTATCTTGTTCATTGGCCTTTTACATCCATAGGCATCACTTCTCTGATATTCGTTGTG
CTCTTTTAATGGATTAATGGTTTGCTTGGTTGGTTCCTCTAGTTAGACTGTAAACTCCTTGAGAGCAGAG
TCTGTATTTTATTAATTACCCACAGTACTAGGTACATAGTTGCCTTCAATAAATATATATTTAATGAACG
AATTTAGTGAATAATGGCTACATTTTTGTACTTCGTTATTATTTATTTATTTTTTTTTTCTGAGACAGAG
TCTTGCTCTGTAACCCACGCTGGAGTGCAGTGGTGTGATCTTGGCTCACTGCAACCTCCACCGCCCAGGC
TGAAGTGATTCTCCTGCCTCAGCCTCCCGAGTAGCTAGGACTACAGGCGTGCACCACCATACCTGGCCAA
TTTTTGTATTTTTAGTAGAGATGGGATTTTACTATGTTGGCCAGGCTGGTCTTGAACTCTTGACCTCGTG
ATCCGTCTGCCTCGGCCTCCCAGAGTGCTGGGATTACAGGTGTGAGCCACCGCGCCCAGCCTTCATTATT
CTTTTGACATTTGATGATAAATACTGTTTATGATTTTCTTTTTCTTTTTTTTTTTTTGGTAGAGACAGTC
TCGCTTTGTTGCCCAGGCTGGTCTTGAACTTTTGGACTCAAGCGATCCACCCACCTCAGCCTCCCAAAGT
GTTGGGTTTACATGCATGAGCCACCTCGTGTGTCCTTGTTCATAATTTTGATGGGCTGGGCGCAGTGGCT
CACGCCTGTAATCCCAGCACTTTGGGAGGCCGAGGTGGGTGAATCACTTGAGGTCAGGAGTTCAAGACCA
GCCTGGCCAACATGGCGAAACCCCGTCTCTACGAAAAATACAAAAATTAGCCGGGCGTGGTTGTGCACGC
CTGTAATCCTAGCTACCCAAGAGGCTGAGGCAGGAGAATCACTTTAACCCGGGAGATGGAGGTTGCAGTG
AGCTGAGATCGTGCCATTGCACTCCAGCCTGGGCAACAAGAGTGAAGCTCCATCTCAAGAACAGTTTTGA
TGAAATTGTCCCTAGATTTGCATCTGGCATCTAGCTCATATCTCAGAGCCAGAATGAAGGATGCTTACCT
CTGGGTTTGGTAAATTTTAGCTTGGTTAATTATGTGTGTGCCTTAATTTTTTTGACCTATTATAAAAGCA
ATATACGTAAATGCAACAGAGAAAATGAAAGTAATCCATAAAATTCTACCTGTGGCCAGCTGCGATGGCT
CATGCCTGTAATCCCAGCACTTTGGGAGGTTGCGGTGGGTGGATCACTTGAGGTCAGGAGTTCGAGACCA
GCCTGGCCAACATGGTGAAACCCTGTCTCTACTGAAAATGCGAAAATTAGCTGGGCATGGTGGTGGGTGC
CTGTAATTCCAGGTACTTGGGGAGGCTGAGGCTGGAGAATTGCTTGAACCCGGGAGGCAGAGGTTGCAGT
GAGCCAAGATTGCACCACTGCACTCTAGCCTGGGTGACAGAGACTCTTAAGAAAAAGAAAAAAAAATTCA
GATACCTGTAACCTAAATAATTAATATTTGAAAAAATTTCATCATTGCTTCTTTCATTCAGTTAATATAT
TTTGAACCACTTGCCATGGTGTTATATATAGACATGTCTTATATATGTGTATATATATGTGTGTATTTAT
TTATTTTTTGAGACAGAGTCTCGCTTTGTCATCCAGGCTGGAGTTCAGTGGTACAATCATGGCTCACTGC
ACCCTTGACTTCCTGGGCTCAAGCAATCCTTCCAGCTTCCCTAGTAGCTGGGACTACAGGCGCATGCCAC
CACACCCAGCTAATTTTTGTAGTTTTTGTTGAGGTGGGAATTCACTAGTCTTGAATTCCTGAGCTCAAGC
AATCCTCCCATCTTGGCTTCCCAAAGTGCTGGGATTATAGGCATGAGCCACTGTGCTCACCCAGTTTTTT
ATTTTTAGATACAGGGTCTCGCTCTGTGGCTTAAAGCTAGATGGTGCAGTGGCACCATCATAGCTCACTA
CAACCTTGAGCGCCTGGGCTTAAGGAATCTTCCTGTCAGCCTCCCCAGTAGCTGGGACCACAGGCATGCA
CCACTGCACCTAATTAAAAAAAATTTTTTTTATAGAGATGGAGGGGGTCTCATTTTGTTCCTCAGGCTAG
TCTCAAACTCCTGGCCTCAAGCAATCCTCTGCTTCAGCCTCCCGAAGTGCTGGGATTACAGGTGTGAGCC
ACGGTGCCCGGCCCCTTATTCTTTTTGAAGGCTGTTTTAAGAATTCCAGATTTCCAGCCATGAATGTGTG
ATTACTAACTAGTCTCTTGTTATATGCCTCATCAACACCACACTTCTCCCAGAAGTTGTGCAGGCATTTA
GTTTATTGTTACTCAGGAATGGATGAGAAAGGATATCAAAGATCTGAACCAGCTGATTACCATCTTGGTC
ACTTGAGAACTCAGGTCTGTCCAATAAACACCCTAATCCAAGGTGGTGTTAAATACATATATATATATTT
TTACTTTACGTTTATTTATTTTGAAAAATTTCAAACCTATAGAAAAATTGAGGCAGTACCATAGTCTTAG
TCCATTTTCCATTACTTAGAATATCCAAAAGTGAGTAATTTATAAAGAAAATTAATTTATTTCTTACAGC
TATGGAGGCCAAGGTCGAGGGGACATATCTGGTCAGCGTTTTGCCATGTTGGTCAGGCTGGTCTCGAACT
CCTGACCTCAAGGCCTGCCTTGGCCTCCCAAAGTGCTGGGATTACAGGCATAAGCCACCGTGCCCAGCCA
CCTCTGAGCACTTTAGATAGTATGTCTTAAGAACAAAGACATAGCCCGGCGCAGTGGCTCACGCCTGTAA
TCCCAGCAGTTTGGGAGGCTGAGTCGGGCGGATCACCTTAGGTCAGGAGTTCGCGGCAAAACCCCGTTAC
TACTAAAAATACAAAAAATTAGCTGGGTGTGGTGGCGTGCACCTGTAATCCCAGCTACTCGGGAGGCTGA
GGCTGGAGAATCACTTGAACCCAGGAGGCAGAGGCTGCAGTGAGCCGAGATGGCACCACTGCACTGTAGC
CTGGGTGACAGAGCAAGACTTGGTCTCAAAAAAAAAAAAGAAAAAAAGAACAAAGACATTTTCCTATATA
ACCACAATGCCGTTATCACACTCAAGAAACTTAACATTGATATGATTGTGTATAATACATAGTCCATATT
CAAATTTTTCTCTCATTTCAAAATTATTCTTTGTGGCCTAAAATCCTGTATCCAATCATGATATTTTTGA
ACAATCCAGACTAGTTGTTGTATAGAAGTAAGTAGTTTGTTTTTTTTGTTTTGTTTTGTTTTGTTTTTTG
AGACAGGGCCTGGCTCCGTTGCCCAGGCTGGAGTGCAGTGGCATGATCACGGCTTACTGCAACCTCTGCC
TCCTGTGCTCAAGCAATCTTGTGCCTCAGCTTCCTAAATAACTGGAATTAGAGGTGCGTGACACCACGCC
TAGCTAATTTTTGTATTTTTAATAGAGATGGGGTTTTGTCATGTTGCCCAGGCTGGTCTTGAACTCCTGG
ACTCGAAGCAATCCTCCCACCTCAGCCTCCCAAAGTGCTGGGATTTATAGGTGTGAGCCACCAGGCCTGG
CAGTTAAGTACATTTTTTAAGGACTAATAAAAATGTAGAAATCTTCGTTATTTCCGGAATCCTTTATGTT
TTACTGCCAGTACTAATGTAGTAATGCCAGACAGTAGTCATGTCTACTGTTCCAGGTTGTCCAAAGGATT
CAGCAGCAAAGGATGAAAGTGGTGGGAAGTTTTGCATAGTGGTCGACCAAGGGCTAGTTTGGTGAGTGTC
TTGCCTATGATAATGGACAGGAAGACACCAGTTATTTTATTTTATTTTTATTTTATTTTATTTTATTTTA
ATTTTATTTTGTGTTGTGTTGTGTTATCTTTGAGATGGAGTCTCACTGTATCACCCAGGCTGGAGTGCAG
TGGCGCAATCTCGGCTCACTGTAACCTCTGCCTCCCGGGTTGAAGTGATCCTCCTGCCTCAGCCTCTTGA
GTAGCTGGGATTATATGCGTGAGCCACCACACCCGACTAATTTTTTGTATTTTTAGTAGAGACAGGGTTT
TACCATGTTGGCCAGGCTGGTCTTGAACTCCTTACCTCAGGTGATCCACCCACCGCAGCCCCCCAAAGTG
TTGGGATTACAGGCGTGAGCCACCACACCCAGCTGACACCAGTTATTTAACTCACATTTCTCTGTTTTCC
TTTTAAAGGTAGTAGTGACACCTTTGAAATCTTTTGGCATTTTCTTTGCCTGTTCTCCCTTATGTGTTAA
GGATAAGCTTACGTGCAGAATGGCTTGTTAAGTTTTTTCTCCTCCGTACTTGGAAATCTCATCTGGTGGG
ACATTAGGGTCAGAATTTTGTATATTATATATTATATATCTATCATGATATATATAATGTATTTTATATA
TTATATATTATATAAATATCATGATAGATATATAATATATAATTATATGTTATATATTTTATATTTATAA
AATAAAAATAAAAAATTGCAATTATTTTCAAATAGTAAAACCACGTAGATAAAATATAATTCTGGCCAGG
CACGTGGCTCATGCCTGTAATCCCAGCACTTTGGGAGGCTAAGGTGGGTGGATCACCTGAGGTCAGGTGA
TATAATATACAACAGATATAATATGTCTATTATATATTATATATAATGTTATATAGATATCTATATTATA
TATAATATATTAATAGATATATTAATATAATATATATTATATATTATACAGATATCTATAAATATATTAT
ATATAATATATTATAGATATATCTATGTATATTATATCACCTGACGTCAGGTGATCCACCCACCTTAGCC
TCGCAAAGTGCTGGGATTACAGGCGTGAGCCACGTGCCTGGCCAGAATTATATTTTATCTACGTGGTTTT
ACTATTTGAAAATAATTGCAATTTTTTATTTTATTTTTTGGTTCTGGAAACATTTGTGTGGACTTACCTA
TGATTGTCAGGTTTACTGATTTGCTGGAGATTTTTTGGGCATGACAAAGGGTTTCTTCTTTGATAAAAGT
TTTTCTGGGTAATGTGATGGTACAGGTTGAGTATCCTTTATCTGAAATGCTTGAGACATGAAGTGTTTTG
GATTTGAGAATTTTTAGCATCTTGGAATATTTACCACATACATAGTAAGGTACCTTGAGGATGGGGCCCA
AGTCTAAACACAGAATTCATTTGTGTTTCATATTTATCTTATATGCATGAAACAAAGTTGTTTGTTTTCT
TTTTTTTTTGAGACGGAGTCTCACTCTGTCGCCCAGGCTGGAGTGCAATGGCATGGTCTTGGCTCACTGC
AACCTCCGCCTCCCAGGTCCAAGCGATTCTCTTACCTCAGCCTCCTGCGTAGCTGGGATTACAGACATGA
GGCACCATGCCCGGCAAATTTTTGTATTTTTAGTAGAGATGGGGTTTCACCATGTTGGCCAGGCTGGTCT
CAAACCCCTGACCTGAAGTGATCTGCCTGCCTCAGCCTCCCAAAGTCCTGGGATTACAGGTGTGAACCAC
CGCACCTGGCCCCAAAATAAAGTTTTGACTCTGTGTGTGTGTGTGTGTGTGTGTGTGTGTGTTTTGGAGA
CAGAGTCTCACTCTGTCGCCCAGGCTGGAGTGCAGTGGCATGATCACAGCTCACTGCAACCTTAGCTTCC
TGGGCTCAAATGATCCTCCCACTTTAGCCTCCCAGGTAGTTGGGACTACAGGTGCGTGCCACTGTGGCTG
AGTAATTTGTGTGTGTGTGTGTGTGTGTGTGTGTGTGTGTGTGTTTTAATAGAGATGGGGCTTTGCCATG
TTGCCCAGCCTGGCTCTTGACCTCTTGGGCTCAAGCCATCTGCCCACCTTGACCTCCCAAAGTGCTAGGA
TTATAGGCGTGAACCACCATGCCCAGCCTTTGACTGTGTTTTGATGGCAACCCATCACGTGAGGTCAGGT
GTAGAAATTTCCATTTTTGGCGTCAGGTTGGCACTCCAAAGGTTTCAGATTGTGGAGCTCCTTGGAGTTT
TGGATTAGGGATGCTCAGCCTGTACATTTGACAGTTTATTTACAGATGCTTGCATCAGTGCTTGGAGCTA
GTCCTCAGTGGTGGGATAGGTGAAAATTATCTCTACTTTGAGTTCTTGATTTGAATTCTTTTTTCTTAAG
CTTTTCCTAAAGTCATGTGTCAGTGTTGATTATTGCCCGGTTAGAGGTAGGATTTGTATTGTTTTTTTAC
ACTTGGAATTTCCTGTTGTCATAAGATCACTGTGAACTCCCTTGTAGTGTTGTACATTCTTAGGACTTTC
ATGCTGAAATACTATGCTTTTATAAAATGGTTGTTGTGTCTTTGCTGAGTTTAAATAGGGTCCAGGCCAG
GCATGGTGGCTCATGCTTACCCTGTCTCAAAAAAAAAACACCCTCAACTAAATATAGTTAAATAGGCCAG
GCTCGGTGGCTCATGCCTGTAATCCCAGCACTTTGGGAGGCGGAGGCGGGCGGATCACCTGAGGTCAGGA
GTTCGAGACCAGCCTGGCTAACACGGCGAAGCCCTGTCTCTATTAAAAATAAAAAAAAAACCTAGCCAGG
TGTGGTGGTGAACGCCTGTAGTCCCAGCTACTCGGGAGGCTGAGGCAGGAGAATCACTTGAACCCAGAGA
CAGAGTGAGACTCTGTCTCTCACACACACACACACACAGATAAATAAATTGGGTTCATAAATGGTGATTC
CAGCACTCACTGTCAGGTATAACTTCAGTCTTTTTTTTTTTTTTTTTTTTTTTTTTTTTTTTTTTTTTTT
TGAGACAGAGTCTCCTTCTGTCACCCAGGCTGGAGAGCAGTGGTGTAGTCTCAGCTCATTGCAACCTCCA
CCTTCTAGGTTCAAGCAGTTCTTCTGCCTTAGCCTCCCAGGTAGCTGGGATTACAGGTGCAGACCACCAC
GCCCAACTAATTTATTTATTTATTATTATTATTATTATTATTTTTTAGTAGAGATGGAGTTTTGCCATGT
TGGTCAGGCTGACTTTGAACTCCTGACTTCATGTGATCCACCTGCCTTGGCCTCCCAAAGTGCTGGGATT
ACAGGTGTGAGCCACTGTGTCCAGCCAACATTATTCACTTTTGTTGTTACATAAGATGTCACGGCCAGGC
ATGGTGGGTCACGCCTGTAATCCCAGCACTTTGGGAGGCTGAGGTGTGTGGATCACCTGAGGTCAGGAGT
TTGAGACCAGCCTGGCCAACCTGGTGAAACCCCGTCTGTACTAAAAATATGAAAATTAGCTGGGATTACA
GGCGGGCACCTGTAATCCCAGCTACTAGGGAGGCTGAGGCAGGAGAATCACTTGAACCCGGGAGGCAGAG
GTTGCAGTGAGCTGAGATCGTGCCATTGCACTCCAGCCTGGGTGACAGAGCAAGACTGCGTCTCAAAAAA
AGAAAAAAATAAAAAAGATGTCAGTTATGGCTATTTGTACTTTATTATACTTCATGTGATGATATATGGT
TCAGCATCAGGGTCTTTTTTCAGTTAAGTTCAGTTGAACCCTTTTCCTGTTTAGAATATTTTTTAAAAGT
ATAAAAAAACTTAATAAAAGAAATGGATGGCTGGGCATGGTGGCTCACCCCTGTAATCCCAGCACTTTGG
TAGGCTGAGGCGGGCAGATCACCTGAGGTCAGGAGTTCAAGACCAGCCTGGCCAACACAGTGAAACTCCA
TCTCTACTAAAAATACAAAAATTAGCTGGGCGTGGTGGCGGCCGCCTGTAATCCCAGCCACTTGGGAGGC
TGAGGCAGGAGAATCCCTTGAACCCGGGAGGCGGAGATTGCAATGAGCCTATTGCGCAACTGCACTGTAG
CCTGGGCGACAAGAAGGAGACTCCGTCCCTCCCATAAAAAAAAGAAATAGATGACTACATTTAAAATATC
TTCCATTTAAATCCATAGTTTACAAATAAAGGAAAAAGCAGTGGAGGAAGAGAGTGAGATAATTACTTCA
TTTTTACAATGTTAAATAATTACAATTAAAATTTTTGTGTACAATTCCACATTTAATAGCAATTTGCAAA
TGCCTTATTGCCAGGTGTGGTGTCTTACGCCTGTAATCCCAGTACTTTGGGAGCCCAAGGTGGGAGAATT
GCTTGAGCCCTGGAATTCGAGACCAGCCTGGGCAACATAGCGAAACTTTTGTCTCTACAAGAAATAAAAT
TAGCCAGGTGTGGTAGCCTGCATCTGTAGTTGGAGGTACTGGGGGAGCTGACGTGAGAGGATTGCTTGAG
CCCAGGAGGTTGAGGCTGCAGCTGACATCAAGCCACTAGACTCTATCCTGGGCGACTAATTGAGACCCTG
TCTCAACGAACAAAGAAACAAAAAAAACCCAAATGATTTACTTACCATTAAATACCAAGAAAAGAATGAC
TGGAGTAAGCTTAGATTTAGATACAACAAAATCATGGAGCTTATCATTTGAAAGGCATTTTTGACTTTTC
ATAGGTGTGTAGCAAGCAAAGTGGTCCTTTTGAAAAATGCAAGTCACATCATGTCACTTTTCTGCTTGGC
CTTTGCCTACTTCTCGAATGGTTCTCCTAGCACATTCTAACTGGTCATTATTCATTGTGTTTCAGCCATT
CTGGCCTTCAACCTATTTCTCAAACACCCCAATCTTGGGTATACTTTAGGGCCCTTGCATTTGCTAGCTC
ATTTACCTGGAAAATTCTGCGGTAACTTCTTGCACATAATAGGTGCTAATAAATATTGGCTGCATGCAAC
CAAAAAAGCTGGCTTCGGTAGTAGAAGTAGTAGAAGAGAACATTACCAAAGTCCTTTTTTTTGAGACAAG
ATTTCCCTCTGTTATTTAGGCTGAGTGCAGTGGTGCCATCACGGCTCACTGCAGCCTTGACTTCTTGGAC
TCAAATGATCCTCCCACCTCAGCCTCCTGAGAAGCTGGGACCACAGGTGTGTGTCACCATGCCTGGCCAA
TTTTTTTATTTGTAGAGAAGAGGTCTTACTGTGTTGCCCAGAATGGTGTCAAACTCCTGGGCTCAAGTGA
TCCACCTGCCTCAGCCTCCCAAAATGCTGAGATTATAGGTGTGAGCCACTGGTCCAGCCTCTTACCAAAA
TCTTTTTTTTTTTTTTTTTTTGAGACGGAGTCTCGCTGTGTCGCCCAGGCTGGAGTGCAGTGGCGGGATC
TCGGCTCACTGCAAGCTCCGCCTCCCGGGTTCACACCATTCTCCTGCCTCAGCCTCCTGAGTAGCTGGGA
CTACAGGCGCCTGCTACCACGCCCGGCTAATTTTTTATATTTTTAGTAGAGACGGGGTTTCACCATGTTG
GCCAGGATGGTCTCGATCTTTTGACCTCGTGATCCACCCACCTCGGCCTCCCAAAGTGCTGGGATTACAG
GCATGAGCTACCGCGCCTGGCCGCCTCTTATCAAAATCTTAAACTTGTTAATTCTTGTATCTCACCTTGC
TTTTATATCAGTTTTTGTTATAGAATGTGCTTTCTTTTTAGGTTATTTTTCTTCTGCTCAGCAAGTTAAC
TTTGAAGAATCTTTTTTTTCTTGCCTTTTTTTTTTTTTTTTGAGACAGAATTTTGGTTTTGTTGCCCAGG
CTGGAGTGCAATGGCGCGATCTCAGCTAACGGCAACCTCCACTTCCCGGGTTCAAGTGATTCTCCTGCCC
CAGCCTCCCGAGTAGCTGGGATTACAGGCATGAGGCCACCAGGCCTGGCTATTTTTATTTTTTATTTTTT
ATTTTTATTTTTTACTAGAGACAGTGTTTTTCCATGTTGGTCAGGCTGGTCTTGAATTCTCGTCCTCAGG
TGATCCACCCGCCTTGACCTCCCGAAGTCCTGGGATTACAGGTGTGAGCCAGCGCACCCGGCCTCTTGCC
CTCTTTAGAGCAGTTCTACATAAATCTGTGGAATAATCTGCTGACGTTAGAAATAAACCTTTCTCAGGGT
GTGGCCATAATCTGGTTCGTTGTAATTGCTGAAGGAAAAAACTGAACCCTTAGGAACAGTTTAAACATTC
TAGGTGGTTTTTAGATATATTATTCCTGTTAGAGTTTTGAACTTTGATCATCTTGTTAGAAAAAAGAGGC
CAGGCACTGTGCCTCATACCTGTAATCCCAGCATTTTGGAGGCTGAGTCGTCAGGAAGTCACTTGAGCCC
AGGAGTTTGGTACCAGCCTGGGCAACATATGGAGACCTCGTCTCTACAAAAAAAAAAAAAAAAAAACAAA
ACCTGTTAGCCAGGCGTGGTGGCTCATGCCTGTGTTCCCAGCTACTCAGGAGGCTGAGATGGAAGGATTG
CTTGAGCCTGGGAAGTTGCGACTGCAGTGAGCTGTGATTGTGCCACTGCACTCCAGCCTGGGCAACAGAG
TAAGACCCTGTCTCAAAAAAAATGAATTTTTGCATTCATTTAAAAATGAATGGTAGCAACATTTTAGATA
ATAACTTATGTAGTCTGTGTGAATCTCATCAAATATATGTGATTTTTAATGCTTGGAATCCTAGAATTTT
AGTGAGGGAGGAGCTCTCAAGTTATCTAATTTACTGTCATTTTACAGATAAAAACTAAACTTCTGATAGA
TGAAAGGATTTATACCATACATCTATATGATAATTGCATATGGCTGAGTTGAGGTTAGAACCCAGGACCC
AATCTTTGATTTGATTTCTTTTCATTATGCCAATTTAGAATAAAATTTTTTCTCTTGGTTTTCTGACCTT
TGGCCACTTCATCTTTTGAAGGATCTTGTAAGTTCCTTGATATTTCTCAAAATGTCAGTTTTATTCTCTT
CAATGAAAGTTCTTGTTAGGTGAAAGTAGCCTTTCATATCCTTAAATGTGAACCTGTTCCTTAGGCTGGA
GTTTTTGTACTCTCCATTATATTGGCATTTATAGTTGTTGGTTTTCTTTTTTTTTTTTCTTTTGAGACGG
AGTCTGTCTCTGTCGCCCAGGCTAGAGTGCAGTGGCGCGATCTCGGCTCACTGCAAGCTCTGCCTCCCGG
GTTCACGCCATTCTCCTGCCTCAGCCTCCTGAGTAGCTGGGACTATAGGCGCCCGCCACCATGCCCGGCT
AATTTTTTTGTATTTTTAATAGAGACGGGGTTTCACTGTGTTTGCCAGGATGGTCTCCATCTCCTGACCT
TGTGATCCTCCCGCCTCGGCCTCCCACAAGTGCTGGGATTACAGGCGTGAGCCACTGCACCCGGCCAGTT
GTTGGTTTTCTTTTCTGTGCCAGATTGAGTGATATATGGTGGTACTAAGAGCACTGGAATAGGAGTCAAG
GACTGAGGTACAGCATAGCACCTCATGTATTACTTGGTATCTCCACTTGTCTCTAACACTGAAGAGGTAT
AATTGGTTGTTTAAAAACTGTGGGTGTAAAACGCTAGTATCCCATGAAATGGAACTGCAGAATATAAGTG
GATGACTCTGAAACTGCTTTTGAAACAAGCATGAGGGCTCAGTTTCTCCATCTCTCGTGGGCTCCACCAT
CACTGTCTGACATCCTGATCTCCTAAGGCATCTTTCCTCCATCCCAGAGCTCTGTTGAAAATCTCTTGGG
CTAAAAACCAGTACAGACCTTCTGATACTCACGTTCTAGCCTTTTTAGATTGTTGTGTGTGAAGAGTAAG
TCAGCTGTGGATATTTTTCTTTCCTATTTTGTGAGGCATTTGGTATTTACTGGAGGCTGCCTGTTTTGTT
GCAAACCTTGTTCCACTGTGTACTATAGGAGGTCTGCTCAGAAAGCCTAGTTAGTAGAGCAGTACCATTT
GCTTTGGTCAATTGTAGCAGCAGTCTGTTTTTTTTTTTTTTTTTTTTTTTTTTGAGACAGAGTCTTGCTG
TGTTGCTCAGGCTGGAGTGCAGTGGTGTGATCTTTGCTCACTGCAACCTCCGCCTCCTGGGTTCAAGCGA
TTCTCCTGCCTTGGCCTCCCAAGTGGGATTGCAGGTGTGTGCCACCATGCCCGGCTAATTCTGTATTTTT
AATAGAGATGGGGTTTCACCCCCAGGCTGGTCTTAAACTCCTAACCTCAAGTGATCCACTTGCTTCGGCC
TCCCAAAGTGCTAGGATTACAGGTGTGAGCCACCGTGCCCGGCCAGCAATCTGGTTTTAAAGGAGGCTTT
GACCCCTCATTTGGTAAAAGGTTCCTTCTTTGACTAATTCCCAAAGGACATCTCGATCTGTCTTCAAAAG
AAGGGGTATTCCGTTGTCAGTATTCCTAGCTAGCTCTGGGAGAAGAGAGAACTTGATTGTTTTCAGGGCC
CTGGTCAGAGTCTGGTTTTATGCCCAAGGGATGAACTGCCTTTTGTCCTTCTACCTCCTGAAGTTCAGAG
CCCTGTGAAAATGAAGTGGTAAAGGTCAAGTAGCAGTAGTTTGAAAAGCACCAGCACTGGGACGATTTTG
TGGTAGCCTAGCAGAGTGGGCAGGATGCTTACTTCAGAGGTGCTAAAGATGTATTTTTTTTTGGAACAAG
AAGGTTATAAATGTTTCTATTAAGTTTGATTTTAAAGCATGAGGGTTCTGGGAAAATGAGAAGATTGTAC
ATACTCTAGGCTTCCTTGGGACTGCATTTGAAAAAAAAAAAACTTTTCTTTGGGTCAGCCAGTATGAAAA
CTGCATTTGATCTCAATCCTTCAGATCAGGCTTTTGGGGAGAGTTAGTTGAAGAATTTGTTTAGTCTAAG
AAAGTTGCTTCTAGCTTAGCTAAATCTGGAAGAAAAAATGGAACCTGATGTTTGAGTGCTAACTGTATTC
CAGACAGACGTTATACTCCTTATATTGGTCCTTTTGAAGCATAAATATGTTTCCAAGTCCTATAATAATG
AGGAAATTGCCTGTGAGAGTTTAAGTAAATTGTACAAAGTAACACTTTTGGTTCAGAGATTTGTTTTTAA
TACAGAGAGGACAGGTTAGGAACTTTGAAGCTTCAGCTAAATGAGTCAGTTCTCAAATGTTTGAGAAGCA
TGCTCAAGGATCTGGAGGAATAGCTTAATTAATTTGTACATCATGAGAGGAGTAGGGAGCAAAAATATGT
ATAATTTCTTCTGAATGATGCTGTCTTTTTTGATGCAGTGGTGCTAAATGGGCTTTGCTTGTATGATACT
TCTTAAATGCATTAGCTAATTATTTTGTTTTAGTCTTCTTTTTTGAAATACAGATGACTCTTTGAGCTGG
TTTTCTTTGGAAAGTTATCTGTCCTGACAATTCTACTTCTAAAACTTCAACTTTTTTTTTTTAGTCTTCA
TTTCCATTGCCACCACCCTATCCTAAATTATCCCGTTATGCTGGATACTGAAATAGCTTCTGCCTGGTCT
ACTTACTTTCACTTCGCCCCCTTACGATCCAGTGCTTTGCTCCTTTTAATCCAGTGGCCAGAGGGATTTT
TTTCAAAACATAATTTGATTATAACAGATCTATGTTTGATACTTATCCATGGTTTCCCATGGCAGTTGCA
GAAAAATCCAAACCTTGTTTGGCCACACTGCCCTTCTTTCAGTAATTTGAATTCCTTAAGCTTTTTCCAG
TTTCAGGATTTCCCATTTTGAACTCTTTCTCCTCCACTTTTTCCTGGCCACAGGTTTCAAGAGCTGACAA
CTTACCTCAGTTCTTAAGAGGATCCTTCATTTATCTCTAAATTGGATTTTCCTGTGTCCAAACCTTACTT
GTTATCACAAGTTTATAATTATTTGGGTGATTGTTTGATGTGTCTCTCCTGACTGGGCCTTAGGGGCAGA
GAATCTGTATTATTTTGTTCTTGTTTATATCCCTAGGAGCTCTTATGGTAGGTATTCAATAAATGTTTGT
TGAATAAAGGCAGTGGAAAAACACATAGAAGAGGTGCTGTGGCAGCAGTTCTCAGTGCCAACAGTTGAAT
TGGTTTACAATAGTAATTTTTAGTGTCAACAAGGAGATGTGTAGTATGGAATGTTTATTAGCAAGATCTT
TTTCTCATGGAATGGAATGTTTGAAAACCTCCAAAGTGTAAGTTTTTTTTTTTCTCAAAAGAAAACAGTC
ATATTTGGGGTTACTTTTTTTTTTGAGACAGAGTCTTGTTCTGTTGCCCAGGCTGGAGTGCAGTGGCATG
ATCCCGGCTCACTGCAACCTCCACCTCCCGAGTTTAAGTAATTCTCATGCCTTAGCCACTGGAATAGCTG
GGATTACAGGTGTGTGCCACCATGCCCAGCTGTTTTTTGTATAATTATTAGAGATGGGGTTTCGCCATGT
TGACCAGGCTGGTCTCAAGCTCCTGGCCTCAAGTGATCTGCCAGCCTTGGCCTCCCAAAGTGCTGGGATT
ACAGATGAGAGCCACCGTGCCCGGCCCATTGTTACTTCTGAAATCGAAATCAAATGCCATATTGATTCCT
GATCTTTTGTGTATGACCTCTCTCTCTCTTTTTTTTTTTTTTTTGCTACCTCTCCCCGAAAGTTTTTAGG
ATCTTTTTTTCCACGATTATGTACCTTGATGTGGGTCTTTTCTCGCCCTTTGTGCTATTAGGTCTAGCAA
CTCATGTGTCTCAGGTCTGGGAAATGATTTTACAGTTTTTCCAGCTGGTGCGTAACTAGCTGTTTGTTCT
GATACTGTCAGTATTTAACTTTATGTTAGAAACATTTCATAATGCAAATGAAAAATTTAAGCCAGGTGCA
GTGGCTCATGCACTTTGTGAGGCTGAGGTGGGAGGAACACTTGAGGTCAGGAGTTCAAAACCAGCCTGGC
CAACATGGTGAAACCCCTTCTACTAAAAAAACAAAAAAACAAAAATTAACTGGGCATGGTTGTGCACCCC
CATAATCCCAGTTACTCAGGAGGCTGAGGTACGAGAATCACTGGAACTGGGAGACAGGTTTCAGTGAGCC
GAGACTGAGACAGAGCAAGACCCTGTCTCAAAATATAAATAAAATACATAAAAAATTTAATATTATGTAT
ATAATATCCAGCCCAGGCTTTTCTATTTGTCATGCCCTGTTTCTTTCACATCATAGCCTTACTCAGATGC
CTGCTGATCCTTGATTGAGGTGTGATGCTTGAGAGTCAGGCTCTTAAATACTACTGGCGAATTCAGTGCA
TTGTGGTTTTGTGACTTCACTTGTCTCTTACAGAAGTGGGGAAGTGGTTTTTACTTCGATGCATGGTTCT
GAAGTAGAGATGTCTATCTCCTGATGCACACTTTAAATAATCCCCATGTTTTCCACTCCATACTCCATCC
TTGTCTGGTTTAAGCTCTTAGCCTGTCTGCAGTTCAGGGGATGAATCTGCTTGGTTCTTTTCAGCATCTC
CCTCTGCAACGCTTAGATCTCATCTTCCTTTGCTTTGCTAACTATTCTCTTTGCTTCTCCATTTGTTTCT
CTGTCATTGATGACTTCCAAGTTATATCCCCATCCTGGATATTCCTTTTGAACGTCAGACTCATATATTC
AGTTACCTCCTCAGTATCTCTATTCGGATATCTAATGGCATCTCAAAATTAACATGTGCAAAACCAACCT
GTTCCACCCTTGTGTTGGCTGTATAAATTAGTGGGAACTCCATTTTTTAAGTTGCTTAAATCAGAAACCT
GGGAGTCATCAATTTTGGAGTCTGTCCCTTTGCTCTTGTGGCACATGTAATCACGAATAAATCCTGTTGG
CTCTATCTTCAGAATATATCTCATTTTATCACTCTCTCTGCCACCAGAGTGGTTTAAGCCACCTTCATCT
CTTAGGTTATTTTAGTAGCTTCCTAACTGGTCTCCTTCCTTCCATTCTTGCCACCTTCCTCAACACCGAA
GATATATTTTATTTATTTATTTATTTATTTTGAGACAGAGTCTTGCTCTGTCACCCCAGCTGGAATGCGG
TGGCTTGATCTTGGCTCACTGCAACCTCTGTCTTCCAGGCTTAAGCAATTCTCCTGCCTCAGCCTCCCGA
GTAGCTGGGGCTGCAGGCATGCACCACCATGCCTGGCTAATTTTTGTATTTTTTAGTAGAGATGGAGTTT
CACCATGTTGGCCAGGCTGGTCTCGAACTCCTGACCTCAGGTCATCTGCCCGCCTCAGCCTCCCAAAGTG
TTGGGATTACAGGCGTCAGCCACCATGCTTGGCCAAATTTTTTATTCCTTTGGGTAAAGAGATGTATTTT
ATTTCCTTTAATTTGGTAATATAGTGAGTTATGTTAATAGATTCTTTTTTTCCAGTGCTCAAGCATCCTT
GCATTCCTAGGATAAATGGTACTTCAACATGATTTTCCTTTTAAATATACATTGCTGGATTTAGGTTATA
AATTATTGAGAATTTTTGCATCTGTATTTACAAGTGTTACATTCTAATTTCAGGCCTTTTCCAGTTTTAT
TATCAAAACAAGCTGTGCTGTTTTTCCTCTCTTGATTCTCAGTAGTAGTCTCTTTAAGATAGTGATTATT
CATTCTTTTTGGTAGATCATGCCTATAAGACCTGGGCATGTATGTTTTTATTGTGGGAATATATTGTTTT
TACTTTCCTTGTTATGAAATATATAATTTATTTTAAAATATGTATATACAGAGTTTGTGTTCCTTTAAAG
AATAATAGCTAAAAATAAATTGCACGCCCCCACATCCAACCATCGTCAAACAAAACATCTTTTACACTCT
ATTTTATTTATTTATTTTCCCCCACCCCTCTAGAGTATACACCCTTTTGGAGCCTCTGAGCATTTTCCTT
TCCCTCACTGCTTTAGAAACCTCTATTCAGATTTTTCATATTAATGATTCTTTTGCTTTTAACCCACATG
TGTTACTGAAGTTTAAGTCCTAAATAATAATTTTTCCTGCTTTAAAATATATTTTTATGTCAATGAAATC
ATACTGGTGGATGTATTTTCTCTGATTTGCTGCTTTTGCCTCTCATTTCTGAGCTTGATCTATGTTGGTG
CATTTATAGAAATACTTCATATTTTCTGCTGTGTATCATCTTATGCTATCAACGTGCCATATGATTTACC
TGTTCTTAGCATGTATTTGGGTTGTTTCTGGTTCTTGCTGTAACAAACAGCTGCATTTAGTGCTTGTGTA
TGTGTCTCCTGATGCATGTGTGCATTCACTTCTCTGGGGTGTATATGTTGCTGGGAATGGGACTGAGTAT
TGTGAGTGTATAATAGTATCTTGTTTTTGTTTTTTTTCCCTCTCAGTTTATGGCATAATTTACGTACAGT
AAAATTTACCACCGCCCCCCTGCCCCGCCTTTTTCTTTTTCTGAAACAGAGTCTCGCTCTGTTGTCCAGG
CTGGAGTGCAGTGGCACAATCTCGGCTCACTGCAACCTTTACTTACTGGGTTCAAGCGATTCTCCTGCCT
CAGCCTCACAAGTAGTTGGGATTACAGGTGCCTGCCAGGCTAATTTTGTAGAGACAGGGTTTCACCATGT
TGGCCAGGCTGGTCTCAAACTCCTTACCTCAGGTGATCCACCCACCTTGGCCTCCCCAAGTGTTGGGATT
AGAGGTATGAGCCACCACGCCCAGCCAAATTCACCCTTTTTAGTGTACAAGTATACAGCTTTGTGAGAGT
TTGATTTCTTTAGTTCAAATTTGTTTTAAAGTTATATTTGGTATTTTAACAGTGCTATTTAAATATATGC
CATATATGGCTTGAATGCAATTATCCTAGTACAGATGGTCCTTGACTTATGATGTTTTTGTGTCCCTGTA
AGCCCACCTTAAGTTGAAAATATTAAGTTGAAAATGCATTTAATATGACTAACCTGCTGTAAATCCTAAC
TTAGCCTACCTTAAATGCACTTAGAACACTTAAATTAGTTTACAGTGGGCAAATCATCAAACACAAAGCC
TATTTGTAATAAAGTGTTGAATATCTTATATAATTTATTGAATACTAAAGTGAAAAACAATAGTTGTATG
AGTAGTCAGAGTATGGCTTCTCTTCAATGCATATCACTTTCATATACCATCGTAAACTCAAAAAATCATA
AGTCAGGGTATGCTGTATATTTTATTGGTGCTAACATACTTCCATTTTAAAATACAGGAAATTCACCTTT
TTTTGTGTATAGTCTTGTAAGTTTTGACAGATCTATATACAGATATGTAATCCCCACCCATGATTCTTTT
TTAATTTAAAAAAATATATTTTTGAGACAGTCTCACTGTCACCCAGGCTGGAATGCAGTGGCATGATCGT
AGCTCACTGCAGCCTCAAACTCCTGGCCTTAAGTGCTCCTCCTGCCTCAGCCTCCCAAAGTGCTAGGATT
ACAGGAATGAGCCAGTGTGCCCTGCCACCACCATAATTAAGATCCAGAACAGTTTCATCATCCCAGAAAC
TTTCCCTGTATCCTTTTGTAGTCAGCTTCTTTTTTCACCCCCAGCTCCTGGTGACCACTGATTTGTTCTC
TGTCCTTGCAGTACTGTCTTTTCCAGAATGCCATATAAATTGAATCCTACATTATATAACACTTTGAGTC
TGGCGTCTTTTACTTAGTGTGAAGTATTTGAGATTTATTTGTCTTGTGATGTGTATTAGCAATTTATTTC
TTTTTTCTTTTTTTTCTTTTTGGAGACAAGGTCTTGCTCTGTCACCCAGGCTGAAGTGCAGCGGCACGAT
CATAGTTCACTGCAGGCTTAAACTCAAGCAATCCTCCCACTTCAGCCTCCTGAGTAGCTGGGACTGCAGG
CGCACACCACCATGTTCAGCTAATTTTTTTTTTTTCTTGGTAGAGACAGGATCTCACTCTGTTGCCCAGG
CTGATTTCAAACTCCTGGCCTCAAGGAATCCTCCCACCTATGCCTCTCAAAGTGCTGGGATTACAGGTGT
GAACCATTACAGTTGGCTAGCAGTTTATTTCTGTATATTGCAGGATAGTATCTCATTGTATAGATGTACC
ACATTTTGCGTATTCATTCTTCAGTTGATAGACATTTGGACTACTTCCTCCTTTTGGCTGCTGTGAGTAA
TGCTGCTGTGAATAAATATTTGTGTGTAAGTTTCTATGTGGACATACAGTTTCATTATTCTTGGGTAGAT
AAACCTTGGAGTAATTGCTGGGTTGGGTGGTAATTTCTGTTTAACTTTTAAAGAAATAGCTAAGCTGTTT
TCCAAAGTGGCTTCACCATCTTAAATTCAAGTTTTTGTTTTCGTTGTATTTGAGGCAGAGCTTTACTCTC
TTGCCCAGGCTGGAGTGCAATGGCAAGATTATATTATAGCTCACTGCAGCCTCGATCTTCCAGGCTCAAG
CGATCCTCTCACCTCAGCCTCCCAGGTAGCTGAGAGTACAGGTGTGTGTCACCATGCCTGGCTAAGTTTT
GATTTTTAGTAGAGATAAGGTCTGGCTATGTTTCCCAGGCTGGTCTTGAACTTCTGAGCTCAAGCAGTCC
TCCTGCCTTGGCCTCCCAAAGTGCTGGGATTGCAGGTGTGAGCCACCAAGCCCAGCCAAGGTTTTTGTGT
TTTAGTAAAATTTCTGCTCTTCCTGGCAATACTTTTTTCTGAAATTTACTTTGATATTGATATAGTCACT
CCAGTTCTTTTCAGTGCTTAGTTTTTGCATGGTATATTTTTTCCATCCTTTTGTGTTATACTGTCTGTTT
CATTATATTTGTAGTTGGACCTTGATTTTTTTTTTTTCCCTAATTCTTTTACTTGGGATGCTTGGACCTA
ATGTGTGTCTAGTTTTTCCTAATGTGTCTTTTACTTGGGATGCTTGGACCATGTGTATTTAATTATTGAT
ATGGTTGGGTTAAGTCCACCATCTAGCTATTTGTTATTTATTTATTTTTAGAGCCAAGGTCTTGCTGTGC
CACCCAGGCTGGGGTGCTAGTGGCATGATCATGGCTCACTGAAGCCTCTAACTTCTGGGCTCAAGCAGTC
ATCTCACTGCAGTCTCCCGAGAAGCTGGGATTACAGGCGTGTGCCACCAAGCCTAGCTGATTTTTTTTTT
TTTTTTCCGAGATGGAGATTTGCTCTTGTTGCCCACTCTGGAGTGCAGTGGCGCAATCTTGGCTCACCGC
AGCCTCTGCCTCCTGGGTTCAAGCAATTCTCCTGCCTCAGTCTCCCGAGTAGCTGAGATTACAGGTGCCT
GCCACCATGCCCAGCTAATTCTGTATTTTTAGTAGAGATGGGGTTTCACTATGTTGGCCAGGCTGATCTT
GAACTCTTGACCTCGTGATCTGCCCGCCTCGGCCTCCCAAAGTGTTGGAAGGCCGAGGCGGGCAGATCAC
GCCTGTGCTGGGAGGCTGAGGCGGGTAGCCACTGCTCCCAGCCCTGAAATCTTTCCTCTTTTCTAAGATA
GACATTTAATGCACACACACATTTTGATATGCTATGTTTTAATTTTCATTCAGCTTAAAATATCTAATTT
TTCTTTTGATTTATTCTTTAACTTACTGATTATTTAGAAGTGTGATATTATATTTTGGGAATAAAAATCC
AGAGATCTTACTGTTACTGATTTTTGATTTAATTCCATTGTGGTAAGAGAACATACCTTATATGATTTGA
ATCCTTTTAAATTGATTGAGACTTGTTTTATGGATCAGAATAAGGTGTCTTGATAAAGGTTCCACGGGCA
CTTGAAAAGAATGTGTATTCTACTGTTGTTAGGTAGAGCATTCTATTAATGTTGATTGGCTTGTTGATTG
ATAATGTTCAAGTCTTCTGTATTCTTACCGATTTCCTATTCTATCAGTTATAGACAGAGGGTGTTAAAAT
CTCAATTCTGTTTTGCTTGTTGATTGATAATGTTTAAGTCTTCTGTATTCTTACCGATTTTCTGTTCTAT
CAATTATAGAGAGAGGTGTTAAAATCTCAATTCTGTTTGTTTTTGGTTTGAAATTTACTTATAAGGTACA
TAAGCATTTATGATTGTCGTCATCTTGATGAATTGGTCTCTGTCATTTTGAAATGACAGAACTTACTTTG
TCTCATAATATAGCTACTCGTGCTTTTCATTTGATTAGTGTTTGGCATATATTTTTATATTTTAAAAAAA
CTTATCACCTGTGTCTTTATGTTTAAAGTGGTTTTTTTTCCCAGGCATCATGTGTTTGGGATCTTGCTTT
TATCCAGTTTGATAGTGTTTGCCTTTTAATTGGAGTTTGTAGACTATTTATGTTTAATGTGATTACTGAT
TTTTTTTTGAGATGGAGTCTCGCCCTGTCGCCCAGGCTGCAGTGCAGTGGCACGATCTCAGCTCACTCCA
ACCTCCGTTTCCCGGGTTCAAGTCATTCTCTTGCCTCAGCCTCCCAAGTAGCTGGGACTGCAGGCACCTG
ACACCACACCCGGCTAATTTTTGTATTTTTAGTAGAGATGGGGTTTCACCATATTGGCCAGGCTGGTCTC
AAACTGCTGACTTTGTGATCCGCCCACCTCAGCCTCCCAAAGTGCTGGGATTACAGGTGTGAGCCACTGC
GGCTGGATGTTTACTGATTTTTATAGAGTTTAAACAACCATCTTTTCTATTTGTCTGATGTGGTGTTTTT
TTCGTTTTTGTTTTTTTAAGATGGACTGTCACTCTGTCGTCTAGGCTGGAGTGCAGTGGTGCAATCTCAA
CTCACTGTAACCTTTGCCTCCTGGGTTCAAGCAGTTTTCCTGTCTCAGCCTCCCAAGTAGCTGGGATTAC
AGGTGCGTGCCACCACACCTGGCTATTTTTTTTTTTTTTTTTTTGGTTTGAGACAGAGTCTCGCACTGTC
ACCTGGGCTGGAGTGCAGTGGCGCGATCTCGGCTCACTGCAATCTCCGCCTCCTGGGTTCAAGCGATACT
CCTGCCTCAGCCTCCCAAGTAGCTGGTATTACAGGCGCCCATCACCACACCCAGCTAATGTTTTGTATTT
TTAGTAGAGACAGGGTTTCACTATATTGGCCAGGCTGGTCTCAAATTCCTGACCTTGTGATCTGCCCGCC
TTGGCCTCCCAAAGTGCTGGGATTACAGGCGTGAGCCACCGTGCCTGGCCTAATTTTTGTATTTTTAGTA
GAGATGGGCTTTTGCCATGTTGGCCAAGCAGGTCTCGAATTCCTGACCTCAAGTGATCCCTGCCCTTGGC
CTCCCAAAGTGCTGGCATTACAGGAGTGAGCTACGCCATGCCTGGCCTGGATTGAGGATTTTTTAGTGGT
TCATTTTTAAGTCCTTTCTTGGCTCATTAGTACTTTGTTATTTTAGTGGCTGCTTTAGTGTTTATTGAGT
TCAGCTTGTGAAACTCAGTCTTTAATATTATACCACTTCACCTATATTACAAGAACCTTATAGCAGTACA
CTTCATTTTCCCTTCAGACTTTGTGCTATGTTTGTCATACACTTCACTTTTACATATGTTGTAAACTCCA
CAATACATTATTATTTTTCATTTAAGTAGTCAATTTTTTTAAAACAATTTTTTTTTTTTTGAGACGGAGT
CTCACTCGGTTGCCCAGGCTACAGTGCAGTGGTGCAATCTTGGCTTACTGCAGCCTCTGCCTCCTGGGTT
CAAGTGATTCTTATGCCTCAGCCTCCTGAGTAGCTGGGATTACAGGTGCGCCCCATCATGCCTGGCTGAT
TTTTGTATTATTAGTAGAGATGGGGTTTTGCCACGTTGGCCCAGCAGGTCTCGAACTCCTGAGCTCCAGT
GATCCGCCTGTCTTGGCCTCCCAAAGTGCTGGGATTACAGGCATGAGCCACCATGCCTGGCCTAAAAAGA
TTTTAAAAGTAAGAAAAAATGTCTTTTACTTTATATGTTTTCCATTTCTGGTGTTCTTTATTGCCTATGA
GCGGTTCCAGATTCTCATCTGCCAGATTTTCTTCAGTTTTTTCTCGTCAGTTCAGATTTTCTTTCTGCCA
GAGGACTTCCTTTAATGTGTTTCTTGTAGTGTAGGTCTGCTGGTGATATCTTTAAGCTTTGGTGTGTTTG
AAAAAGTACTTCCTCTTGTTTTTAGAAAGATATATTCCTTTGATACAGAATTCTAGGTTGACAGGTTTTT
TTCATTGTGTACTTTAAAGATGTTGCTCCACTGTCTCTAGCTTGCATTATTTCTCCTGTAAAATCTGTTG
TTATTTTAATCTTTGTTCTTCAGCGAGCAAATTTATCTTTTTTGCCTTTTTTGGTTAATTTTTAAGATTT
TCTTTTTATCACTGCTTTTTCAGCAATGTGTTTATTATATACTTTGTTGTAGTTTGCGTCATATTTCTTG
TGTTTGAATTCTCTTGAGCTTCATGGATGAAATTTACGTATTCAGCTACTATTTCTTGAAATATTTTTCT
TTTGTACTTCCCCACCGCATCTGGAGTCTGTAATTACATGTATATTTGGCTGCTTGAAGTAGTCATACAA
CTCACTGATGCACTGTTATGTTTTTTAGTCATTTGATTCTCTTTGTTTTGGGTAGTTTCTACTGATCTTC
ATTTCACTAGTCTTTATCTTTCCCATGTTAAATCTGTTACTCTTATCCAGAATTATACCTTTTATCCTTA
AAAATTCAGTTTTTTTCTTTTTTTTGAGACGGAGTCTCATTCTGTCACCCAGGCTGAAGTGCAGTGGCAT
GATCTTGGCTCGCTGCAACCTCCGCTTCCCAGGTTCAAGCGATTATCCTGCCTCAGCCTCCCAAGTAGCT
GTGATTACAGGCATGCATCACCATGTCTGGCTAGTTTTTGTATTTTTAGTAGAGACAGAGCTTCACCATG
TTTGCCAGGCTGGTCTCAAACCCCTGACTTCAGGTGATCCACCTGCCTCAGCCTCCTAGAGTGCTGGGAT
TACAGGAATGAGCGACCACACCTGGTTTGTTTCTTAAAAAACATCTTATATTTCTCTGCTTAACGTGCTC
AATGTTGAACATGTGAAATATAATAACTTTTCATAATCTCTTCTGATTCTGTCTCTATATCATCTTTGGG
TCTGTTTTTATGTATTCTATTTTTCTTGTCATTATGGGTCAGATTTCCCAGTTCTTTGCAATCCTAATAA
ATTCTGTTTAGATGAAAGACATTGTGAATTTCACTTTGGGTTGTGGATATTTTTGTATTCATGTTGGTAT
TTTTGAGATTTGTTCTGGAGTGCAGATGAGTTAGCTGGAAACTAATTCTTTTGAGGCTTGCTTTTTAGCT
TTGTTAGGTGGGGCGTATCCTTTCTGTGTTTATTTTTGTAAGGGTATACAGATATACATGTTTTCTTATT
TCCCTCTTTATTTATTCTTATTTAATCTGTAACAATCTTTTTCCTTATATTCGTAACATTCTTAGTTAGG
GAGTAATTACCTCTTCCCTAACTGTTGCATTAGCTTCTTAATTGGCACTCTGGTTCCCAGACTTTTTCTG
TCTCTAATGCAGTGTACAGACTACTGTCAGAATAATTCTAAAGCACAATTCTGATATTGTCACTGTATTG
CTTTTCAGTGCCTACAGCAGTAGGATTCACTTAAAAAATTATTCCCCTAATAGTAATATAAAAAATTTTT
TTGAGCTCAAACTCTCATTCTCTCTCAGTATACATATTTGTTTATACATGAGGTCACATACTGCTTACTA
AGTTAGCATTTTTTATGTGTTCATTTTGAAATAACTCTGAACAGTGGTATCCAATCTTTTGGCTTCCCTG
GGCCACATTAGAAGAAGAATTGTCTTGGGCCACACATAAAATACACTAATGATAGCTGAATAGCTAAAAA
TTAAACATTGCAAAAAAAAAAAACAAACAAAAAAAACTCACTGTGGCTTTTTTTTTTTTTCCCTGTATTT
TTCTTTTTCCCGAGATGAAGTCATGCTCTGTTGCTCAGGCTGGAGTGCAGTAGTGTGATCTCGGCTCACT
GCAACCTCCGCCTACCAGGTTCAAGCAGTTCTCCTGCCTCAGCCTCCCGAGTAGCTGGGATTAAAGGGGT
AAGCCCCCATGCCTGGCCTTTTTTTTTTTTTTTTTGTGGGGTGAGCGACCATGCCTGGCTTTTTTTTTTT
TTTTTTTTTTTTTTTTTTTTGGTAGAGATGGGGTTTCATCATGTTGGCCAGTCTGGTCTCGAACTCTTGA
CCTTGTAATTCACCTGCCTTGGCCTCCTAAGGTGTTGGGATTACAGGTGTGAGCCACCACTCCTGGCCTT
TTTTTTTTTTTTTTTTGAGATGGAGTCTCACTTTGTCACCCAGGCTGGAGTGCAGTGGCATGATCTCGGC
TCGCTGCAGCCACTGCCTCCCGGGTTCAAGCAGTTCTCCTGCCTCACCCTCCCGAGTAGCTGGGATTACA
GGTGTGTGCCACCACTCCCAGCTAGTTTTTTGTATTTTTAGTAGAGGCAGGTTTCACCATATTGGCCAGG
CTGGTGAACTCCTGACCTCAAGTGATGCACCCACCTGGGCCTCCCAAATGCTGCTGGAATTGCAGGCGTG
AGCCACCATGCCAAGCCAATTGTGTTTTAAGAAAAGTTTATGAATTAGTATTGGGCAGCATCGAAAGTTT
ACAAAGTTTGCGTTGGGCCGCATTGAAAGCTGTCCTGGGCTGTGTGCGACCTGCTGGCCGCAGGTTGGAC
AAACTTGCTCTGGGACATAAGTTTAAAAGTTATAGGCCAAGTGCCTTGGCTCACGCCTGTAATGTCTTTG
CATTTCATCAGTTTTTCCACTGTTATCTTTCTGTTACAGTACACGGTCAGAATACCACATTGCATTTAGT
TGTCATGTTTCCTTAGATGTGAAACAAATTTACCAATTGTAGTAGGGTATTTACGTATAGATCGGTTCGT
TTGTAGCCTTATAGTATCTAGTTAAAACACTGTTATAAAGTTATTGTTATGTATCCTTTTGGGTGTGGTT
TCTTTCCTTAGAAAAATACATTTAAAATTCATTCATGTTGTTAAATGAATCAATAGGTTGTTTCTCTTTT
AAAAATTGAGATGAAATTCACATAACAAAATTAACCATTTTATTTTTTTAATTTTATATATATTTTCTGA
GATGGAGTCTCACTCTGTAGCCCAGGCTGGAGTGCAGTGACGCGATCTCGGCTCACTGCAACATCTGCCT
CCTGGGTTCAAGCGATTCTCAGCCTTAACCATTTTAAAGTGCACAGTTAAGTGGCATTTAGTACATTCAC
AGTGTTGGGTAACCATCTTTATCTGGTTCCAAAACATTTTTTTTTCTTTGAAAGAAAACCCCGTATTTAT
TAAGCATTTTTCTCTCTGTCCTTTTCCCTTCACTCGCTTCTGGCAAACACCAGTCTGCTCTGTTTCTGTG
GATTTACCTGTTCTGGATATTTTATACAAATGGAATCATAGCGTATGTAGCCTTTTGTGGCTGGTTTTGT
TTTTTTTGTTTGTTTTTTTTTTTGTTTTTTTTTTTTGAGACGGAGTCTTGCTCTGTCGCCCAGGCTGGAG
TGCAGTGGCGGGATCTCGGCTCACTGCAAGCTCCACCTGCCGGGTTCACGCCATTCTCCTGCCTCAGCCT
CCCAAGTAGCTGGGACTACAGGCGCCCGCCACTACGCCCGGCTCATTTTTTGTATTTTTAGTAGAGACGG
GGTTTCACCGTTTTAGCTGGGATGGTCTCGATCTCCTGACCTCGTGATCCGCCCGCCTCGGCCTCCCAAA
GTGCTGGGATTACAGGCGTGAGCCACCGCGCCCGGCCTGGTTTTGTTTTAGTAAGATACTTTTGCTGGAT
TAAGAATAAGGTTGGGCCAGGGGTGGTGGCTCATGCCTATAATCCCAGCACTTTGGGAGGCCGAGTTGGC
CCAGCCTGGCCAACACAGTGAAACCCCATCTTTACTAAAAATACAAAAATTAGTCAGGCATGGTGGCGGG
TACCTTGTAATCCCAGCTACTTGGAAGGCTGAGGCAGGAGGATTGCTTGAACCCGGGAAGCGGAGTTTGC
AGTTAGCCAAGATCATGCCACTGCACTCTAGCCTGGGCGACAAGAGCAAGACTCGTTTCAAAGAAAAAAA
AAAGAATACGTAGTTGACAGGTTTTTTTTTCTTTCAATACTTTAAAGATGATATACCACTGTCTTGTCAC
TCACCTTGTTTCTAACAAGAAATCTGCTATCTTTGTTCTGTATTTTGCATGCTTTTTTTTTCCTTGCTGC
TTTTATTATTTTCTCTTTATTACTGGTTTTGAGCATTAGATTATGATATGCCAGTATGGTTTTTCTTGTG
TTTATTGTGCTTGTGGTTCATTGATGTTCTTGGATATGTCGGTTTATAGCTTTCTTCAGGTTTCGGATTT
TTTTTTTTTTTGGCCATTTTTTTCAAATTATTTTCCATTTAATTTCTCTTTTCTTTTTAGTCAGGTACTC
TTTATCCAAACATTAGGCTGCTTGAAGTTGCCCCATAGCTTACTGATAGTTTTTGTTTTAACCTGTGTTT
TATTTGAAAGATTCTATTTCTGTGCCTTTGAATTTACTTTCTTTTGCAGTGTGTAATCTGAAGTTAGTCA
CATTTAGCATTTTTAAAAATCTCACATATTTTACTCTTTTTTTTTTTTTTTTTTTTTTTTTTTTTTTAAA
GACTGAGTCTCACTCTGTTGCCTGGGCTGGGGTGCAGTGGCACAATTTCAGCTCACTACAACCTCCGCCT
CCTGGTTTCAAGCGATCCTCCCACCTCAGCCTCCCGAGTAGCTGGGACTACAGGCACCCGCCACTACGCC
TGGCTCATTTTTGTATTTGTAGTAGAGACGGGGTTTCGCCATGTTGGCCAGGCTGGTCTTGAACTCCTGA
CCTCAAGTGATCTGCTTGCCTCAGCCTCCCAAATTGATGGGATTACAGGTGTGAGCCACCATGCCCGGCC
ATATTTTACCTTTTTTTATCGTTAGAATTTTGATTTGGTTTAAAAAAAAATCTTTCCTGTCTCTACTTAA
GTTTTTGAATATATGAAATGCAGATATAACTCTTATAATGTCCTTGTGTACTCATTCTAACATCTGTGTC
ATTTCAGGGTTGGTTGCAATTGACTATTCTCTTTAGTATACTTTTTTTTCCTTGCCTCTTGCCTGGTGAT
CTTTGATTGGATTTCAGACATAGAGAGTTTTACCTCGTTGGGTGCTGGGGATATTTGTATTTCTACATAT
GTTCTTTAACTTTGTTATTGGATGCAGTTAAGTCACTTGAAAACCGTTGATTCCTTTAGGTCTTGTTTGT
AAGATTTGTTAGCTGGGAACGGTGTAGTAGTTGGTTTATGGCTAATTCTTCCTCACTTCTGAGACAAGAT
TCTTCCCAAATCTTGAGGGTTGTCTTGAGATTTCCAGTCTAATAGCTACTTGAGTGGGTAAGGGCAGAGT
ACTGTTCATAATTCTTTTGGGTGTTGTTTTCCTCTCCTTGCTGCCCATGGTCTTGGGTAGTTTCATCACA
CTATGGGCCACTCAGTACTCAGTGAGTACCTCTCTAGATCTCCTCAGCCCTCTACTGTGTGGTATTGTCC
CATGAATTCTAGCTGCCTAAGTCCTGCACTCTGAGCTGAGCTCGCTGTCTTCAATTTTGAGTTCTGCAGC
CTCCATAGGGCTTCTGCTTCTGCATGGTGCCCAGGAAACTCTCTTCAGGCACTAAGTTTGGCCATCATAG
GACTCATATCTTTGTTTCTCTTCCTTTGGGGATTACTGTCTCTCATTGCCTGCTGCGCAGCATCTTGAAA
GCTATAGTTTCATGTTTTGTTAGTTATTTTAGTGGTTTAAGGTGAGAGGGTAAATCTGGTCCCTGTTGTG
CTTGAAAAGTTTTTTTCAACTTTTCTTTTAGAAATAATTTTAGGTTTACTAAAAGCTGCAGAAATAATAC
AGAGTTCTGAGGTGTCTTTCACCCACTTCCCCCATGATAACATTTTACATAACCATAGTATAGTTATTAA
AACTAGGAAATTGACATTGGTATAATAGTGTTAACTAAACCTCATGCCTTATTTCATATTTTCACATGAA
CTATTTTGAGCTGGCATGGTTAATATACAGTTCTATGAAGTTTTATCACGTGTATAGATTTCGTGTAATT
ATCACTAGTCAGGTTATAGAAGTATTCCATCATTTTAATGAAATGCCTTTGTACTACTCCTGTGTAGTCA
TCATGTCCTCCTTCCAGCCTCCACCACCAATCAACCAACCAACTTTAATCCTGGCAGCTACCAGTTGTCT
GAATTTTATCACTTTCGCTATAATTTTATCTTTCTCAGAATGTCATAATACAGTAGTCCCCGTTATCTGT
GGTGATATGTTCTAAGACCCCCAGTGGATGCCTGAAACCAGCTATAGTACTGAACCATATATGTGTGTGT
GTGTGTGTGTGTGTTTTTTCCCCCCCCCCCTTTTTTTTTTTGAGACGGAGTCTCACTCTCGTCACCCAGA
CTAGGGTGCAGTGGTGTGATCTTGGCTCACTGCAATCTGCACCTCACAGGTTCAAGCGATTCTTCTGCCT
CAGCCTCCTGGGTAACTGGGATTACAGGTTCGTGTTACCACGCAGGCTAATTTTTTGTATTTTTAGTTGA
GGTGGGGTTTCACCATGTTGGCCAGGCTGGTCTCGAACTCCTGACCTCAAGTGATCCACCCACCTTAGCC
TCCCAAAGTGCTGGGATTACAGGCGTGAGCCACTGCACCTGGCCTACTATACATATACGCCTATGATGAA
ATTTAATTTATAAATTAGGCACAGTAAGAGATTAACAATAATAAAAAATTATAACAATATACTATAATAA
AAGTTATGTGAATGTTGTTTCTCTCTCAAAATACTGTAATATTTTCAGGCTGCAGTTGACCATGCGTAGC
TGAAAGCAAGGGTAAGTGGTGACTACTGTAAATAGAATCATATAATATGTTACCTTTGAGATTGGCTTTT
CTTTTTTAACTCGGCAAAATGCCTTTGAAATCGATCCAAGTCCTATGTATTAACACTGTTTCTTTTTATT
GGTGAGTAGTATTCTATTCTTGGGCACACTACAGTTTACCCATTCTTCCATTGAGAGCATTTCTCCCCCC
TAAATTACAAACAGAGATGGAGTCTTGCTTTGTGGTTGGTCTCCAACTCCTAGGCTCAATCTATCCTCCT
GCCTCAGCCTCCCAAAGTGCTGGGATTAGAGGCATGTGCCACTGTGCCTGGCCCCACTGAAGAACATTTG
AGTTGTTCCCAGTTTGGGGCTATTACAAATAAAGCTGCTGTGAACATTTGCATGCAGATTTTAGTGTGAA
AATATTTTTATTTCTCTAGGATGAATACCTACCTATGAGTAGAATTGCTGGGTCGTATGATACATGTATA
TTTAACTCTGTAAGAAACGACTACACTGTTTTCCAGAGTGGTGGTAGTTTTTCATTCCCACTAACAATGT
ATAAGAAATCCAATTGTTCTGCTTTTTTATTAGCACTTTAGTATTGTCAGTATTTTTCCTTCTGTTCATT
GTAATAGATGCATAGTAGTATCTCATGCCTTAAATTTGCATTTCCGTAGTGGCTAATGATGTTGAATATC
TTTTCATCTCCTTAGGTTCCATTTGAATATTCTCTTTGGTGTAGTTTATGTTCAAGACTTTTCTATTTTC
AAATTGGATTTTTTCTTACTGTTGAGTGTTCTAGACACAAGTTATTTGTTGGATACGTGATTTGCAAATA
TTTTCTCCTAATCTTTTAATTTGCCTAAGAGTGTGCTTCATAGAGCAAAAGGTTTGAATTCTTATGAAGT
CTAATTTATCTATTTTTAAATTTTATAGTTTTTTTTGGTGTCATATCTAAGCACTCTTTGGCTAACAAAA
TTTTCTCTTTTTTTCTAAAAGTTGTATGCTTTACATTTAAGTCTGTGATTGCATTTGAATTAATTTTTAA
ATTAAGCATAAGGTTACATCAATGTTCATTTTCCTTTTTGGGGGACCAATTATTCCAAAACCATTTGCTT
AAAATGATTATTCTTTATTTATTGAATTGCTTTCTCACCTCTAGCAAAAGTCAGTTGGCCATATTTTGTG
AAAAGTTCTATTCCTACGCACACTGTTCTGGCTCATTGACCTACGCATCTGTTCCTCTGTCAATACCGTA
CTGTCTTGATTACTGTAATGTAGCTGTATAGTAAGTCCTAAAATCAGGTAGTGTTATTCCTCCACCTTCG
TTTTTCTTTTTCAGAGTTGTTTCAGCTATTCTGGTTTCTTTGTCTTTACATGGAAATTTTATTTATTTAT
TATTTATTTTCTTGAGACAGCCTCAGTGTTTTGCCCGGGCTGGGGTGTGGTGGTATGATCATAGTTCACT
GCTGCCTGGAATTCCTGGGCTCAAGGGATCCTCCTGCCTCAGCCTTCCGGGTAGCTGGGACTACAGATGT
ACACCACCACTCCCAGCTAATTTTTACTTTTTTTGTAGTGCTGGGGTGTCACCCTGTTGCCCATGTTGGT
CTCAAACACCTGGGCTCAAGTGATCTTCCTGCCTCAGCCTCCCAAAGTGCTGGGATTAAAGGCATGAGCC
ACCACAGTCAGCTGTGTATGAATTTTAGAATCATATTATCTACAATAAAGTTTTGGTGGAGTTTCAATAG
TAAATGCATTAAACCCATTGGTTAGTTTGGGGAGAAGTGACATCTTGGCCATGTTGTCTTCCAGTTCATG
GATATAGTATGTTTTTTTACATTTATTTAGGTTGTCTTTTTTTTTTAAAGGTACATAATAATTGTACATA
TTTATGGGGTGCATGTGATATTTTGATACAGGTATGCAGTGTATAATGATCAAAGCAAGGTATTTAGGCT
ATCCATCACCTCAAATATTTATCATTTCTTTATGTTGGGAATTTTTCAACTCTTCCCTTCTAGGTATTTT
GAAATATAAACAAATTATAGTTAACTACAGTCACCATACTGTGCTGTTGAACCCTGGAACTAATTTCTTC
TATCTAACTGTATGTTTGTACCCATTGACCAACCCCTCTTTATCTCCCTGCCCCCTTCCCAGCTCTGGTA
ACGATCATTTTACTCTCTGCCTCCACGAGATCAACTCTTTTAGCTCCCACATAGGAGTGACAACATGTGA
TATTTATCTTTCTGTGCCTCTTCCTTTACTTAACAATGTATTTAGGTCATCTTTGGTATTTTTATCAGCA
TTTCATAGGTTTTAGCATGCATATTTTGTACAGTTTTGTTAGATATATACCTAGTTTTTTTTGGTAAACT
ATTATAAATGAAATTGTGCTTTTAATTTTTGTTTCTAATTGCTGATTGCTAATATATAGACATTATTGAT
GTTAGTGTGTTGATCTGTATTCTGTGACCTTTATAAACTCACTTATTAGTTCTACAAGTTTTTCTCCTAG
AATCTTTGGGATTTTCTCTGTTAGACCATCATATCATCTGTTAATAGGGCCGGTTTAATATATTCCTTTC
TGATATGTGTACCTTTTATTTCCTTTTCTTGCTTGATTGACTAGCTGGGACTTGTTTGCTGTGTCGAATA
TGAGTGCTTAGTGTGGACATTCTTGCCTTATTCCCGCTTTCAGATAGAAAGCCTTTAGTCTTTCACCACT
AGTATGATGTTAGTTATAGACATTTTATAGATGGTCTTTATCAAGTTGAGGACGTTTTCTTCTATTCCTA
GTTTACTGAGAGTTTTTATCATGAATGTATTTGTTGAATTTTGTCAAAGTTTCTGCATTGATATAAATAA
TTATGCTTTCTTTTCACATCTTATTGTTTTAGTTTGCATTTCCTTGATGATGAATTCCTTTTCATATGCT
TACTGACCATTTTATCATCTTCCTGCGTGAATTTTCTATTAGTTTTGCCCATTTAAAAAAAGTTTTTATT
TTGTAGCAATTTTAAGCTTTCATAAAGTTCTAAGTACAGTACAAAGAACTTTATTTTTTCCTGACCCATT
TTAGAGCCCTGTTGTTCCTTACTATTTTTATTTATTTTTTGAGACAGGGTCTGTCACTCTGTTGTACAGA
CTGGAGTGCAGTGGTACTATCATAGCTCACTCCAGGGCATAAACGATCCTCCTGCCTCAGCCTCCTGAGT
AGCTGGGACAAGTGTGTGCCACCATGCCTGGCTAATTTTTTTAATTTTAATTTTTTGAACTTCTGGGCTC
AAGCGATCCTCTTGCCTCAGCCTCCCAAAGTGTTGGGATTATAGGCATGCACCACCGCACCTGGCCCTAC
CCTTAATACTTTAGTATTAATGTTTTAGTATATTTGCCTTAAAGTTAATGACATTCTCCTTGCTACCTGT
CCACAATACATTCATAAAATTCAGAAAATTACACTGAGTCATTATTACCATCTAATCTTCAGATTTCATT
AAAGTTTAGCTAATTGTCCATGTAATGTCCTTTTTGTCCAAAGTATCTAGGTCAGAATTATGTGTTGCAT
GGAGTTGTCATATGTCTTTGGTCTCCTTCATTCTAGAACAGTTCTTTAATCTTTCTTTGATTTTCGTGAC
CTTGACTCCTTTGAAGACTACAGACCAGTTATTTTGTGGAATCTTTTTTAGTTTGAATTTTCTCATAATT
GGATTCAGGTTTTGCATCTTTGTCAGGTATAGCAGAGAAGTGATGCTAGGTTCTTAGTGTGTCCTGTCAG
GTAGCTCTTTATTTCAGTTTTTTCCTATTATTGATTATCTCTTTGATCACTTGGTTAAGGTAGCATCTGC
CATCCTTCATCACTATAAAGCTCCTTTTTACCCCCACCCCTTTTTTTTTGAGATGGGGTTTTGCTCCTCT
TGCCCAGGCTTGGAGTGCAATGTCTCAAAAAAAAATTAATAGTAATATATACAGTCATAGTTAGAAGCCA
AGATCTAGTTTCTAAATGTTTGCTGCTGTTGGAGTGTTGGTGATCCCAGTTCCTTTCAGTGGGAAGGGGC
TAAGGAATATATGGGTATATTTACATACATACACATATGTTCACTCAATCTATATTTATATCTGTCTTTA
TCTAAATATGTTGAAAACTATGAGCTTACCCCAATAATACAAGTTCTAATCCAGTCATTCTAGTTTTTTC
CCTTTCCATATGTATAAGTCCTTTTTCCAACAATAATAAATCTGGGCTGGGCGCAGTGGCTTACACCTGT
AATCCCAGCACTTTGGGAGGCTGAGGCGGGTGGATTGCTTGAACACAGGCGTTCTAGATCAGCCTGGGCA
ACATGTCAAAACCCCATCTATATGGGGAAAAAAAAAAAAGTTAGCCAGGTGTGGTGGCACATGCCTGTAG
TCCCAGCTACTCAGGAGGCTGAGGTGGGAGGATCACTTGAGCCCAGGAGGTCAAGGCTACAGCGAGCCGA
GATTGCACCACTGCACTCCAGCCAGAGCTACTGAGTGAGACCCTGTCTCAAATCTATTCCCTAATATATT
TACCTAATTATTTAATCACCCACTCTTCACTGTTGCAGCCCTTTCTTCATGCAGATGTCCTCTTTACTTT
GCTTGGACTCCAACACCCTGTGTTGGGTTGGCTCCACTCTCATCCCATGGATGAGTTCACTCAAGCTCTG
ACCAGTCACATCAGGGTGTTCTCCCATATACATGCCCTTCTCAACCCGCGTGGGCTTCAATACTCTGTCC
TTTGTCATTCCCTGTTTGGGCATTTCACTCATCTTGATCTCCAACTCTCACGTCATACCACACTCAGATA
CACACAGATACCTTCTGTGCTCCACTCAGGCTCTGACTCTCTACTCTTGTCTACCTAGCCTCAGGCCTCC
ACTTTTGTATGGATGCCTACCTTGCTGTGTACCATGTAATGGAAGGGAAGACTAGTTTGGGGGAAGAGAA
GGGCAGCTCTTCCCATTTAAAAAGATTGGTTTGTCTTTTTGTTTTAGGTTTATAGAATTGAAATATATAG
GTGGGCTTGGTGGCTCACGCATGTAATCCCAGCACTTTGGGAGGCTGAGGCAGGTGGATCACTTTGGTTT
AGGAGTTTGAGATTAGCCTGGTCAACATGGTGAAACCCCATCTCTACTAAAAATACAGAAAAATTAGCTG
GGTGTGGTGGAGGGTACCTGTAATTCCAGCTACTCAGAAGGCTGAGGCACAAGAATTGCTTGAACCTGGG
AGGTGGAGGTTGCAGTGAGCTGAGATCGTGCCACTGCTCTCCAACCTGGGCAACAGAGCTAGACTCCGTC
TCGGGGGGAAAAAAAAGAATTGAAATATATGTATACACACACATATACACACTCTGGATACATACTGTAT
CCCGAGTATGTGTTTTGCAAATATTTTCTCCCAGTCTGTGGCCTGCCTTTTATTTCTTTTCTTTTTTTTG
AGACAGGGTCTTCCTCTGTCACCCACGCTAGAGTGCGGTAGCACAGTCACAACTCACCGCAGCCTCAACT
TCCTGGGCTCAAAGTGATCCTCCCACCTCAGTCTCTTGAGTAGCTGGGACTACAGACACGTGCCACCACT
CCTGGCCCATTATGTCTGTTTTTTGTAGAGATGACGTCTCACTATGTTGCCTAGGGTGGTCTCAAACTCC
TAGGCTCAAGTGATCATCCCACCTCAGCCTCCCAAAGTGCTGGTGAGCCACCATGCCTGGCCTGGCCTGG
CCTGCCTTTGCATTTTTATTAAAGTGTCTTTTGATTAGAAGTTTTTTTGTTTTGAGGAAGTCTGTTTTAT
CCACATTAAAACAATATGTTTTTAGGAACTTTTGCCTATCTATTAATAGTGCACAAAAATATTTTTTGGC
TTTATATTTTTGGCTCTTAGGTGTGCGATTTATGTCAAATTAATTTTTATTTTTGAGAGGGAGTCTAGCT
TTGTCACCCAGGCTGGAGTGCAGTGGCACAATCTTGGCTCACTGCAATCTCCACCTCCCAGGTTCAAGCA
ATTCTCCTACCTCAGCCTCCTGAGTAGCTGGGATTATAAGCACTTGCCACCATGCCTATTTTTCTATTTT
TAGTAGAGATGGGGTTTTACCATGTTTGCCAGCCTGGTCTTGAACTCCTGACCTCAGGTGACCTGCCCGC
TTCAGCCTCCCAAAGTTGCTGGGATTACAGGCTTGAGTCACCATGCCTGGCCTCAAATTAATTTCTATGT
ACGGTTCGAGGTAGTCATTCAGGTTTGCGTTTTTACGTGTTTATCTTGTTTCAGCTTTATTTGTTGAAAT
GATGTTCCCCTCTTTATTGAATAGCTTTGGTGCCTTTGTCAAAAATCAGTTGAGTCCATACATAAGGATA
TGTTTTTGTACTCTGTTCTGTTCCATTAATCTATCTATTTTTATACCAATATTTTACTCTCCTGATTTTA
GTAAGTCATGAGATTGGATAGTGTTAGTAGTGTGAATCCTCTGTGTTCTTCCTTCTTAAAATTGCTTGGA
CTGCTTTTGGTTCTTTGCATTTATTTTTCTTTCTTTTTTAGAGACAAGGTCTTGCTCTGTCGGCAGGCTG
TAGTGCAGTGACGCAGTCCTAGCTTACTGCATCCTAAAACTTCCGGGCTCAAGCCATCCTCCTGCCTCAA
GCTCCCAAGGCGCTGGGATTATGTGTGTGAGCCACCATGCCCAGCCTTCTTTGCATTTTCATATAAATTT
TAGAATCAACTTGTCAATTTTTATCAATTAAAAAAATTGGGATTTTGATTAGGATTATGTTGACTTTGTA
GGGAGAATTAACTTCTCAACAGCATTTTTTTTCAATCCATGAACATGGTATAATCTCCAGTTATTATCAT
CTTTAATTTTTCTTAGTGTTTTATAGTTTCCAATGAGGTCTTGCATGCCTTAACATTTTTTTCTCAAGTA
TTTTTATTTTTTTAATTTTTTAAATTAATTTATTTTTTTGAGATGGCGTTTTGCTCTTGTTGCCCAGGCT
GGAGTGCAGTGGCGGAATCTTGGCTCACTGCAACCTTTGCCTCCCAGTTTCAAGCGATTCACCTGCCTCA
GCCTCCCTAGTAGCTGGGATTACAGGCGGGCACCACCATACCTGGCTAATTTTTTGTATTTTTAGTAGAG
ACAGGGTTTCACCATGTTGGCCAGGCTGTTCTCAAACTCTTGACCTCGTGATCTACCTGCCTTGGCCTTC
CAAAGTGCTGGGATTATAGGCGTGAGCCACCATGCCCAGCCTCAGCTATTTTTATTTTCGATGCTATTAT
ACATGGAATTTGTTTTAAAAATTTGTTTCCCAGTTTTTTGCCCTAGCAGGTATATAGCTGATTTTTCAAT
AACTTGTAATTTTGACATAATTTCAGACACGATTGTATTTGCTTTATAATTCTAGCTACATGTTATTTTT
TTCATGTACTAAAGTGCAGACATAATGCCCCCTAACCTCTAAATAGTTATTAACAACAAGTACATTCTCT
TTCATAACCACATTGCAATTATCAATGTCAGAAAATTAATGTTGATATGGTACTGCTTGCTGTCTAATGT
GCATACTTTGAGCTGGGCTTGGTGGTATACACCTATAGTCTTAGCTACTCAGGAGGCTGAGGTGGGAGGA
TCGCTTGAGCCTAGGAGTTGAAGGCTGCAGTATGCCATGATCAACCCTGTGAATAGCCACTGTACTCCAG
CCTGGGCAACAATAGTGAGATCCCTATCTTTAAAAAAAAAACTGCATATTTTACTGAAATTTTACCTATT
CTTTTCTAATGCCAATTATAAAGCAAAATTCTGGTTGAGGATCTCATCCTTGATCACATGGTACATTTGT
TTGTCAAATCTCATTTTCTTTAATCTGAAAAAGTTCTCACAAACATTGTCTTTTATGACTTAATTTTTTT
TAAAGTACAGACCATTTATTTTGTATTAATAGTAAGTTCTCCAATATGGATTTGTCTTGATGCTTTTCAT
TATTAAAGTAATGTTTTTGACAGGAATACCTCAGCATGTCCTCCTCAGTACATTTTATCAGGAGACACGA
TACCTGTTTGTCTGATTTCTGATAACGATAACTTCAAACACTTGGTTAAGGTGGTGTCTGCCTTATTTCT
CTCCTATAAATAGTACCTTGTAGAGAGATACAGTAGTCCCCTTTATCTGCTGGGGATGTTTCAAGACCAG
TGGGTGCCTGAAATTACAGATAGTCCCAAGCCCTATATATACTGTGTTTTTTCCTATGCATATGTACTTT
TGATAAATTTTATTTTATTATTCCTTTATTTAGTTTTTAGAAATACGGTCTTACACCGTTGCCCAGCCTG
GTCCTGAATGAACTTCTGGCCTCAAGTGATCCTCCTGCACTGGTCTCCCAAAGTGTTGGGACTACAGGTG
TGAGCCACTGTGCCCAGCCCCCCAAGTTTAATTTCTAAATTAGGCACAGTAAAAGAATAACAACAATAAC
TAAGAATAAAATATAACAATTGCAACAATGTACTATAATCAAAGTTATGTGGATGTGATCCTTCTTTCTC
TCTCAAAATACTGTAATTTTTTGGACTGCTGTTGACCACAGGTTATTTAAACTGTGGAAGGTGAAACTTC
GGGTAGAGGGCACTTACTGTACTTTGAGACTATATGAATAGTCTCATGTACTTTCATTCACTGGTTTTAG
CATCCACTGATGAAGGATTCCTACCTAAAACAGTTAGTACGCTGAGGCTGGGTGCAGTAGCTCACACCTG
TAATCCCAACACTTTGGGAGACCGAGGTGGGTGGATCATTTGATGTCAGGAGTTTGAGACCAGCCTTGCC
AACATGGTGAAACCCTGTCTCTGCTAAAAATACAAAAAAATTAGCTGGACATGATGGCGCATGCCTGTAA
TCTCAGCTACTGGGGGAGGGTGAGGCAGGAGAATCGCTTGAACCCTGGAGATGGAGGTTGCAGTGAGCTG
AGATCGTGCCACTGCACTCCAGCCTGGGCAACAGAACAAGACTCTGTCTCAAAAAAAAAAAAAAATAAAA
AAAATAAAAAAAAATTAGTACTCTGAGAGTTGTTGTCAAGTGGTGATTTTTTTTTTTTTTAAGTTCCACC
ATTTATCTGACATCTATTAGTTGTGGTTTTTCAGTAAGGGGGAGAGCTTTAATTTATTCCCCCATTTGTT
TATTCGTTGTGAATTTATGGATTCTTTTTTTTTTTTTTTTTTTTTTTTTTGAGATGGAGTCTCATGCGCT
CTGTCACCCAGGCTGGAATGTAGTGGCACAATCTCGGCTCACTGCAACCTCTGTCTCCTGGGTTCAAGTG
ATTCATCTTCCTTAGCTTCACGAGTAGCTGGGGTTACAGGTGCCTGCCACCATGCCCGGCTAATTTTTGT
CTTTTTAGTAGAGACGGTTTCACCATGTTGGCCAGGCTTGTCTCTCCAACTCCTGGCCTCAAGCGATCCA
CCCGCCTCAGCCTCCCAAAGTGCTGGGATTACAGGCATAAGCCACTGTGCCCGGCCATGGATTCCTTTTT
TTTTTTTTTTTTTTTTAAAATATGAAGACTCGCTCTGTTGCCCAGGCTGGAGTGCAGTAGTGCGATCTTG
GCTCACTGCAGCCTCTGCCGCCTGGGTTCAAGTGATTCTCCTGCCTCAGCCTCACAAGTAGCTGGGATTA
CAGGTTTGTGCCACCACACCCAGCTTTTATATTTTTAGTAGAGATGGGGTTTCACCATGTTGGCCAGGCT
GGTCTCGAACTCCTGACCTCAAGTTATCCACCCGCTTTGGCCTCCCAAAGTGCTGAGATCATAGGCATGA
GCCATTATGCCCAGCCCTACCCATGGATTCTTATTTTATTATTGAGTTATAATCTTACTATCATTTTTAA
TGTACAAATTGTCGCTGATTTGGCCAGTGGAATTGCATGTACTCCAGCTTTTGTGTCCTTTTTATGTGTT
TTCCTCATTCCTGAACACTTCTTTACTTACCTGCTTTCTGACCCAGCAAGATGTTCTGGGCTTAGCTTAT
ATTTTCCCAGCCCCACTTCTAGAATCATCCATTTTCCTGGGGAGCTCTGGTTTCTTTTAGCTTAGAATAG
TATGCATGTATACATCTGTACCTGTTTCTCTCTTTGTCTGTCTATAGGTAAATATAGTATTCATACTAAT
ACCTTGAAATTAGTCTCTTACTGGGGGTTTCATTCTAACTTTACCTTTCTATATGTGCATCTTGCTACTC
CAACATTGAACAACCTGGCTTCTATTATGTACTTTGTATCTACCTACTTGCTCAGTTTTAGTATAAACAC
TTAGTAGTTTTATAATTGTTAACCCATTCCACTGTGGAAGAACAAATCTAACTAGAACTCAGTATTTGTT
TATAGTTCATTTAGGTTTTTAGCCTAAGGATATATAGTCAAAATACTTGAAGAATTACTTGGGTTAGATC
TGTCCTTGTGCCCCTTCAGTGTGGTTATGTTATTCACTTTAAGTATATGTCAGTAAAAATTTTAAATGTA
TCACAACCACTTCCTCTTTTTAGTGCTGTGGGAAAACAAGCACTGTTGCATTACCCTTAAGAAAAGAAAT
AAATTTTTTTTTTTTTTTTTTTGGAAACAGGTCTCACTCTGTCTCCCAGGCTGGAGGGCAATGGTGGGAT
CGTAGCTCACGTCAGCCTCCCAAGTAGCTGGGACTACAGGCATATGCATCCTGGCTGGCTAATTTTTTTT
TTTTTTTGCAGACATGGGGGTCTCACTATGTTGCCTAGGCTTATCTTGAACTCCTGGGCTCAAGTGATCC
TCCCGCCTTGCCATTCCAAAGTGTTAGGATTATAGGCGTGAGCCACTGTCCCCAGGTCAAGAAAACAAAT
GGATACTTTTTTTTGTTGGGGGATTTGATACTATTAACAAAATTATATTTGCATCTACCTTTTGACCGAT
TGTTTCCATTTGATTATTCAAGGCAGTAGTATTTGTAGTTGCAGAATATTAAAAGCCATCTAAATGCTGG
TGGTACATGAGGGAGTGGTTATACTTTGATACATCAACATCATGGAGTATCATGCAGCTGAGAAGAAAAT
GAGGAAGTTTTCATGAAACGATAGAGTAATTTCCAGAATATATTGTTAAGTGGAAAATTGAAGAGCAAAA
TACTCTCTGAGGCACGCAGATTTTGTTGTTGTTGTTGTTTAAGGATGAAGGATAAATAAAACAGATGTGT
ATATTATCATTTGTACAAAAAGAAACACAAGAAGAATACACCGGAAACAATGAGGTTGGTTAGGGGACTA
ACAGGAATGGAATGGAGAGTTGGAGGGGGATGGAAATGGAAGGGATGGTGGGGAGCAGTGTCACTTACTC
TGAGTATAACCTTTTATATAGTTTTGATTTGTAATCATGTTATATTTCACATAATTCAATATGTAGATAA
AATAACAAAGATGGGAAATAAAAAACAAAATGGGTTACAAACAAAAGATGAAACCCAGTGCTATTAACAG
CTGTACAATTCTTGATGTGTTCAACGTATTTCATTCTTCTTTCTGAATGTTTGTTCCTGGAGCCATCTTT
TTTCCTGCTCTCCGCATGGACAGTTTGTGGTGTAAGGTGGCCAGCCATCCTGGTTTGCCCACGATTGAGG
GGTTTCCCGGGGTGTAGGACTTTCAGTGCTAAAACTGGAAACTCCCAGGCAAACTGGGGCCTGTTGGCTA
CTCTTTTCACGTGCATGTGGCTGTTTTCCTCGGACTTTCCTTATATGTACCTTGGGAATTCTCTTCACTT
TCTCCTGGTATGTCCTGCCTTTCCTAGATCTCACACAAATCTCATTTATTGTTACTCCTTTAGTTGTGTG
CGCATTCTCTACACAATACATTTATGTAGAGCAGATTGACGTAGAGTTGCTTCTAGAGAACAGGTGCCTA
GGAGGTAAATTGAGGCCTTACATGTCTTTATTCTGTGTTCCACACTAGATTGTTGGTTGGGCTCAGAATT
CCAGAAGATGTTACTCTTACTACTACATTCTTCAGAGCTGCTTTTGAGAAACATGCCATTGTGATTTCTG
GATATCGTGTGTTTTTTTTGACCGGAAGATTTTAGGATCTGTTTCTCTCCTGTGTTTTGAGATTTCATGA
TAATGCTGTGGAATGGGTCTTTTTCATTCATTATGCTGAGTGTTTTGCCTTATTAAACTCGGAAACTGGA
GTCTTCTCATTTGGGGAAAATTTCTTGTATTATTTCTGTCTTTTGGCTTTCTTCCTCTGGACCTTCTATT
TGTTGGATGTGGGACTCTCTTAATGGAGCTTCTTTTTCTTTTTTTTTTTTAATCAGTTCTCTAGGAGATA
ATCTGTTGTTTTTGGCCTCTCATTTTTAATTTCTAGCAGATTGTTCATGTTCTTTAATGTTGCTTTATTT
TTTTAAAAAAATATTCTGGCTGAGCTGTAGTACGTGTCTGTGGTCCCGACTACTGGGGAGGCCAAGGTGG
GAGAATTGCTTACGACCAGGAGTTTGAGTCCAGCCTGGGCAACATAGCAACACCCCATATCTTATTTTAA
AAAAAGCACTCTATGTTTGTTTTGGGCCTCTTTGAGAACATTTACTATGAAATTTTATTTATCTTTGTTT
CCTTTCAGTTCTTCTTTTGTTTATTGTGATTATCTTTCATATAACAATGTGCTTAAATAGCTGGTGGTTC
TTGATAGGTCACTCAACAGTGAGCAAACTGATGGATCCTGTGTTTGTGAGCAGTGTTTATTGGCTGTGAT
AGAGGGTGGGGACCTAGATGCTTTTAGGGGATCCTCCTGTTATCCATTTCTGAAAGTGTTTTCTCCTAGG
CTGGTCACTTTTTCCAGGAGGAGAAATCCTTTCATCTCTTGCCATCTACATGACAGTAGGCTGTGGTTGG
GGTCATAGTACTACTACTATTTGGGGAGCTTTGGGAGCCAAGGTGGGGAGGGAAGCTAGTGGTGTCATGA
TTTTCTGGTGTTTTTTGTTTGTTTGTTTGTTTGTTTGTTTGTTTTGTTTTGTTTGAGATGGAGTTTATCT
CTTGCCGCCCAGGCTGGAGTGCAGTGGTGCCATCTCAACTCACTGCAACCTCTACCTCCTGGGTTCAAGC
AATTCTCCAGCCTCAGCCTCCCTAGTAGCTGGGATTACAGGTGCCCGCCATCACCCCTGGCTAATTTTTG
TATTTTTAGTATAGATGGGGTTTCGCCATGTTGGCCAGGCTGGTCTCGAACTCCTGACCTCAGGTGATCT
TCCTGCCTCAGCCTCCCAAAGTGCTGGGATTACAGGTGTGAGCCACCATGCCCAGCCTGCGTCATGATTT
TCTATTGTTGGTCTTATTTAGAGTGTGGCATCTCACTCCATGCCCCATCCCTTGAGCTGTGCCTCTAGTC
TTCTGTTCCAGAACTTCCGTGGTTCAGTTTAATCAGAATATAAATCTGTTTTCCTCTGGAGTGGGAGAAG
GGTGTATTGCCTTACTGTTTAGGGACAGGAAGGAGACCTGAGGGGGTCTGACTGTTTTATTCAGAGTTTG
AAACAGTTCTCCTGTTCTCAGCCCCTGCCTCTCAACTCTTGTCTGCAGTGGTTCCTGGTGCCTCCCACTT
CCCTGCGTGCTCCGGGCTTTTGAAGCACTGGCTTGCTTTTTGTGGCTTTTCCCTCTGCAGCCATAAACTT
CAACTTTCCTTCTTTGCTGAGTCAGTTACTTCTTACCTTCCTGCTCAACCTGGTCTTTCAGATTTCTGTT
GACATCCCTCATCTACTGCCTTTTTCCTTCCCCTTACTCTTTGTCTTTATGCATGTACACTTTTACTGTT
CCCTTATAGTCATTCTAGTGAACCCTCAGGAAGATGCAAAGAAGTATATGTTCATTTTGCCATGTTTATC
CACAACCCCCAAATACCATTAAACAAAAGAAAAGAAAAAATATTTAAAAAAGAAAACAAAAACAAAAACA
AAACAAACCCTTAATAAATGGAGAGCAGCTATGCCATGTTGTTGATTAAGAAGATTCTATATCGTAAAGA
TGTCAGTTCTTCCCAATTTGATTTGTAGATTCAATGCAGTACACTACAAATTTTGAAAATTGAGATATAA
TAGTTATATGTATTTTGGGGGTACAGGTGATATTTTGATACCTGTATGCAATACATAGACATTTTTCTTT
GTGTTGGGAACATTAAAATTCTTCTTTTCTAGTTATTTTGAAATATATAGTAAATTATTGTTTACTATAA
TTTTCCTATTCTACTATTGAATATTAGAACTTACTCCTTCTATTTAACTGTAATTTTATACCCCTTAACC
AACTTCTATTTTGCCATGTTTAAATATGGAAGCTCAAGCGTTTGTACAGAGAAAGTGGAAAATGCCCTTG
GCTTTTATGCCAAACCCCAATCCCTCTGTCCCCACCTGCCACCCTGGCCTGTTGCCCCTGTTCTTCCCAG
TAGCTACAACCACTATCATTAATTTAACGTGCATCCTCCTATTCATATGGAGGTATGTGGTATGTTTGTT
TATATAAGTGTATTTTATTTTCTTTAATGTTTAATAGTGTACCATATCAATATAGCTACATTTTATTTAT
CCAGTCCCCTGCTATGAATATGTGCGTTTTTCACTGTTTTCTAATCCCTGTTATAAACACTGCAGCAACA
CTTATCCTTAATCTCCATGTACAAACGTGTGAGAATTTCTCTAAGGGACTATTCCCTTACCTTTTCATAT
TGTATGCTTGGGTAAAATGGAAGTGACTTGGAGGCTCTTTCTGTGGGGGTTAGTGAAAAGATTTCATTAC
ATTTTCTTTGCATTTTGTAATTATGATGAGAAAAATATAAAACTTATAAATCTTTTTTAAAAGTTTTGAG
AAACATGCTTTGTTACGATCATGACATCAAGTTTTATGCTTAAAAAGTCTGTAATTCCTTGTGTTTAATG
ATGAACTCTTCAAATTCCTGATACAGATCTATAGTCCCTTATTAACAATTCTGAAATCCAAAATATTATG
AGAACTGAATTTTTTTCCCCTTGACTGATTTAGTAGTAAAACCTGACCTGTGTAAACTCATTTTGTGTCA
AAACTCTGATGTAGATTGACAGGTCTAGGTTAGCTTATTTGTTTCACTTGGTCTTTGTCTACATTTTATT
TTTTTAAAAATTAATGTGTTTGATTATGGGTTGCTGGTGGTGCTGATACACAATATATGCATATATTAAC
ATTCTAAAATTCAAGAAATTCTAAATAAAAACACATCTAGACCCAAAGATTTCAGGTAAGGGATTGTGGA
CCTGTGGTCACCATTAGCTAGAAAGTTTCAGCTGTGAGTGGCAACTTACAAGAATTTCTTTCTGGTTGAT
AGAAACTCCTTTCTTATTATTAGTAAGAACTCAGACAATTTCTTTACGTTATATTCCAAGAGTGTGTTCA
TTTTTTAAATAACAGCTCTTCCTTTTCTTTCGTTGACATCTATAATATTGAAATTGCTTTTGATAATGCA
AATGGATTTCACATTCAAGAGAACCTTTTTATGTTAAGTATTTTGTTTAAATGGAGAAGCCTTTTCTCAA
GCATGTATGGAGATTGCTTTCCTTGTTTTAATTCTTCTTTAGGATTTTCATGAGAAAATTAAAACATCAT
TAATGGACCATTTTAGCCACAGTTTTCCAAAGTTGCATTTTGACTTTGGATCTTTAAACTTTCTGTATAC
TTTAATAGGTATGTGTAGGTTTAAAAAAAATTTTTTTTTTTTGATCAGAATCTTTTGTTTTAGGCCAGGT
GTGGTGGCTCACACCTGTAGTCCTAGCACTTGAGGCTGAGGAGGGTGGATTGCTTGCGCTCAGGAGTTCA
AGAGCAGCCTGGGCAATATGGCAAGAACCCCATCTCTACAAAAAATAAAAAAGTTGGCCGGGCGCGGTGA
CTCATGTCAGTAATCCCAGCACTTTGGGAGGCTGAGGCAGGCGAATCACGAGGTCAGGAGTTCGAGACTA
GCCTGGCCAACATGGGGAAACCCCGTCTCTACTAAAAATACAAAAAATTAGCTGGGCATAGTGGCAGGCG
CCTGTAACCCCAGCTACTTGGGAGGCTGAGGCAGGAGAATTGCTTGAACCCGGGAGGCAGAGATTGCAGT
GAGCTGAGATGGAGCCACTGTACTCCAGCCCAGATGACAGAGCAAGACTCTGTATCAAAAAAATAATAAT
AATAAATAAATACAATAAATAAATAAATAAATTAGCCTGGAGTGGTGGTGCATGCCTCTAGTTCCAGCTA
CTTGAGAGGCTGAGGTGGGAAGATCACTTAAGCCTGGGAGGTCAAGGCTGCAAATGAGCTGTGATAGCAC
CGTGGCACTTTAGCCTGGGCCCGCACAGTGAGACCCTGTCTCAAAAAACAAAAACAAAAAATACGTTTTA
GAACTATCAGTTAAATTTTGTAAGCCATTGTTTACCTACTTTAATTGGGATTTAATTCATAGACCATACA
GTTCACCAATTTAAAGTATACAATTCAGTGGCTTCTAGTATATTCATGAAGTTATGCAACCATCACCATG
GTCTGATTGCATAATATTTCCATCACCCCAAAAGGAAACCCCAGTAGGTCATTCCTATTCTCACCTCCTC
CTAGTCCCTAGCATCACCAGTCTACTCATTTAATTACTAAAACTTTCTAAAAAGCAACTCCTCTCATCTT
GTTCTTCAAGAGTATCTTGGCTCTTTGTGACCCTGTGCTTTTCCATGTAAAATTCTGTAAAAGATCGTGT
TGAGAGCTTTATTGGAACTGCATTGAATCGACAAATCAAAATGGGAAGAATTGACATCTTTTTTTTTTTT
TTTTTTTTTTTTTTTTTTTTTTTTTTTTTGAGACGGAGTCTTGCTCTGTCACCCAGGCTGGAATGCAGTG
GCACAATCTTGGCCTACTGTAATCTCCGCCTCCCAGCTTCAAGCGATTCTCCTGCCTTAGCCTTGCGAGT
AGCTAGGATTACAGGTGCACGCCACCATGTCTGGCTAATTATTGTATTTTTGGTAGAGATGGGGTTTCAT
CATGTTGGTCAGGCTGGTCTTGAACTCCTGACGTCGTGATCTGCCCGCCTTAGCCTCTCAAAGTGCTGGG
ATTACAGGCTTGAGCCACCATGCCCAGCCAAATTGACATCTTTATGATATAAAATCTTCTTAATCAAGAA
CATGGTATAGCTCTCCATTTATTTAGGGTTTGTTTGTTTTTGTTTTTTTGAGACAGGGTCTTGTTCTGTC
TCTCAGGCTAGAGTGCAGTGGTGCGATCATAGCTCGCTGCAACCTGAAACTCCTGGGCTCAAGCAATGCT
CCCACCTCAGCCTCCCGAGTAGCTGAGACTACAGGTGCGCGTCACCATGTGCAGCTAATTTTTGAATTTT
TTTGTAGAGCGGGGGTCTCCTTACGTTGCTCATGTTGGTCTTGAACTCTTGGGCTCAAGTGATCCTCCTT
CCTCAGCTTCCCAAAGTGCTGGGATTACAAGTGTAGCCAACATGCCTGGCCTTACCAAGGTTTTAAGCCT
TTCTTGATAAAGTTTTATGTTTTCTTCATAAAATGATGATATTTCTTTTGTGGAATTTATCCCTGGGTAA
CTTACGTTCGTTTTTGTTATAAATTATATTTTTGTTATAAATTATATTTTTGTTTTCTTTTTTCTTCAAC
CCTGGCAAATGGATATATTTTCTTTTTTTTTTTTTTTGAGACAGAGTTTCGCTCTTGTTGCCCAGGCTGG
AGTGCAGTGGCACCATGTCAGCTCACTGCAACCTCCGTCTCCCATTCAGGCAATTCTCCTGCCTCAGCCT
CCTGAGTGGCCGGGATTACAGGCGTGCGCTACCACGCCGGGCTAATTTTTGTATTTTTACTAGAGTCAGG
GTTTCACCATGTTGGCCAGGTTGGTCTTGAACTCCTGACCTCAGATGATCCACCCACCTCGGCCTCCCGA
AATGCTGGCATTACAGGCATGAGCCCCCACACCCGGCTGATGGATAAATTTTCTAATTGATGATGTTAAT
TGGTGATGTTAGTATCACTTGATTTTTCCTTTTTTTTTTTTTTAATGTGATCTCAAATGTCACAATGTGG
CCAAAATCTTTTACTAGGTCTGAGTCTCTTGGATCTTTAATGTAAATAATTATGCTATCTGGAAATTATG
ACATGCTTTTCTAATCCCTTCTTGTTTCTATTATGCATTGAAAGGAATTCTAGTATACTGTTGAATGGTT
GGTGTTTTCTTAGTTTTTCACAGCCTTCAAGTCAGCCTTCTCCTAAGAGATAGTACTTATTTTTTGCTCA
GGAAAGATATAAGGAAAGGCATAACTGATATTTTGCTTTTTTTTTTACTTCAGAATCTAATCTTGGGCAG
TTTAAACTTTATCAATTTAAATGTTAGCTCTCCTTTGCTCAGATTTGCCACATTCGTAGTGTCTTGTATT
TTTTAAAACATCTGACCTGTGGGAGAACATTTTATTTTGGTTTACTTATTACAGATGGTTTATTTTTTTT
CAACATTAGCTTCTTTTCTAGTTACCTTGCCAAACTAGTATACGTTATGCATTTTAGAAACACTTATTTG
TATTAATAGATGAACGTGAGTAAAAAGTATACAGCAAGTCTTCTTTGTACTGTTTTACTCTTGCAAAAGT
AGAATGAAAAGCTTTTGCAGCTTTCCTGTAAAATTGAATTTCTTTCCATATAAAAATGTTCAAAATGTAT
TTGTAAAAGGAATACGTACTCATCATCAGTATTAAAAAGGTTCAGAATAAAGAATCCCTACAACTACAGT
TTTTAAAAGACAACTCAAAAAAAAAATGGGTGAAAGACTTGAACAGATACTTAAAGAGGAAGATATGAAT
ATGGCCAATAAGCACATTAAGAAGTGCTCATGCAGCCATAAAAAAGAATGAGTTCATGCCCTTTGCAGGG
ACGTGGATGAAGCTGGAAACCATCATCCTCAGCAAACTAACACAGGAACAGAAAACCAAACACCCCATGT
TCTCACTCATAAATGGGAGTGAACAGTGAGAACCCATGGACACAGGGAGGGAAACATCACACACCGGGGC
CTGTTGGGGGGTGGGGGGAAAGGGGAGGGAGAGCATTAGGAAAAATACCTAATGCATGCGGGGCTTAAAA
CCTAGATGATGGGTTAATAGGTGCAGCAAACCACCATGGCACATGTATGCTTATGTAACAAACCTGCACA
TTCTGCACATGTATCCCAGAACTTAAAGTAAAATTTAAAAAAAAAAAAACTTCAAAAAAAAAAAAGTGCT
CAACATCTTTAGACATTAGGCAAATTAAAATGAAAATCATTGAAATCTAGCACTCTGAATTAAAATTAAA
ACCACAATGAGGTACCATTATACTCCCACTAGAATGGCTAAAATAAAACTGCCAGCTTCAAATAATGGTG
AGGATTTGGATCAACTGGAACTCTCCTCAAGTGCTGTTGGGAGTATAATTTTTTTTTTAATAAAGTTAGC
TTTCATCCATCTTATGACTCGGCATTTCTACTCCTAGATAGTTATTCAGGAGAAATGAAAACGTCTCCAA
AAATTTACAAGAATGTTCATAATAGCTTTATTCATAATAGCTAGAACCTGGAAATAACCCAAATGTCCAT
CAAGAGAGAATATACAAATGGTTGTATATGAGAATATTAGTCTGTAATAACAAATAATGAACTACTGATG
CATGCAACAATGTAGATGAATAGCATAAACAGGCTGAGTGAAAGAAGCCAGATACAAAAGAATATATATG
TATGAGTCCATTTACATGAAGACCAAGAACAGGCAAAAGTACGTTATGGAGATAGAAGTCAGAACAATGA
TTGCCTTCGTGGGGTGAGAGGGGTGGTTTCTTTTCTTTTTTTTTTTTTTTTTTTTGAGACGGAGTCTGGC
TCTGTCACCCAGGCTGGAGTGCAGTGGCGTGATCTCGGCTCACTGCAAGCTCCGCCTCCCGGGTTCATGC
CATTCTCCTGCCTCAGCCTCCCGAGTAGCTGGGACTACAGGCGACCGCCACCACTCCTGGCTAATTTTTT
TTTTTTTTTTTGTATTTTTAGTAGAGACGGGGTTTCACCGTGTTAGCCAGGATGGTCTCGATCTCCTGAC
CTCGTGATCCGCCTGCCTTGGCCTTCCAAAGTGCTGGGATTACAGGTGTGAGCCACCGCGCCCGGCCGAG
AGGGGTGGTTTCAAATGGGGGTTGATTAGGAAGGGGCACAAGGAAACTTTTGGGGGTGTTGGAAATGTTC
TATATCTTGATATGCATTTGTTAGAACTCATTAAATGGTACACCTAAGATCTGTGCATATCACTGTGTTT
AAATTAAACTTTAATTTAAAAAAATAGGGGAAGGATAGGAGGAGGTATACATTGATCCAAGGGCATTCTC
CTACGTAAGCACAATACAGTTATCACACTCAGAAAATTTAACATTAATGCAGCATTACCATCTAGTATGC
AGTTCACATTCAAATATCCCAGTGTCTTGAAATTTGCTGAGAGTAGATCTTAATTGTCTTCACACTCCCC
ATCTCACACTCTCCCTCTCATGGTAACTGTGTGGTGAAGGATTGGTTAATTTTTTTTTTTTTTTTTTTTT
TTAAAGACAGAGTCTTGCACTGTCTCCCAGGCTGGAGTGCAATGGCATGATCTTGGCTCACTGCAACCTT
TGCCTTCCAGGTTCAAGTGATTCTTGTGCCTCAGCCTCCCGAGTAGCTGGGATTACAAGCGTGCACCACC
ATGCCCGGCTAATTTTTGTATTTTTAGTAGAGACGGGGTTTCACCATGTTGGCCAGGCTGGTCTCGAACT
CCTGACCTTGTGATCCACCCGCCTTGGCCTCCCAAAGTGCTGGGATTGCAGGCATGAGCCACCGTGCCCG
GCCTATAGAATTTTTTATATGTAGGATTTAGTGAAAGATCACCCATTATTAATTTTTTTAAAAGCTTCAT
GATGAAAATTATTCTTGTAAAATGTTAAAATCTTGAATCCTAGGGCATGTTTTCTGGCACCTGTTATTTT
CAGTAACCCATTTCGTTTTTGTCAGACATATAGGGTTAATGTATGAAAAGTCAATTATGTCAAACTTATA
GAGTTTATATGGAAAGATTAATGATCTTTTACAAATGCAGAATTGGCTGGATGTGGCTAATTGTAGAATT
GGCCTGTAACCTAGCACTTAGGGAGGCTGAGGTGGGAGGATCGCTTGAGTCCAGGAGTTTGATTTTTTTT
TTTAATTTTTTATTTTTTGAGACAGTCTCACTCTGTCGCCCTGGCTGAAGTGCAATGGCATGATCTCAGC
TCACTGCAACCTCCACCTCCCAGGTTCAAGCGATTCTTGTGCCTCAGCTTCCCAAGTAGCTGGGATTACA
GGTGTGTGCCACCATGCCTAGCTAATTTTTTGTGTGTGTGTGTATATTTAGTAGGGGGGGTTTCATCATG
TTGGCCCAGGCTGGTCTCGAACTCCCAGCTTCAGGTGGTCCCCCCATCTCGGCCTCCCAAAGTGCTGGAT
TACAGGTGTGGGCCACAATACCCAGCATTTTTTATTTATTTATTTATTTATGTATTTGAGATGGAGTTTC
ACCCTGTTGCCCAGGCTGGAGTGCAATGGCATGATCTTGGCTCACTGCAGCCTGCATGTCCCGGGTTCAA
GCGATCCTCCTGCCACAACCTCCCGAGTAGCTGGGAATACAGGCATGTACACCACACCCAACTAATTTTT
GTATTTAAGTAGAGTCAGGGTTTCATTATGTTGGTCAGGCTGGTCTCGAACTCTGGACCTCAAGTGATCT
GCCTGGCTTGGCCTCCCAAAATGCTGGAATTACAGGCTTGAGCCACTGCACCTGGCCAAGCCCAGGACTT
TGAGACCAGCCTGGGCAACAGACCAAGACCCTGTCTGTACAATAAAAATTATGCAGTATTAGAATAGCAA
ATCTTACTCTTAAAAGTTTTGTTTTCCTTGTCTTTTTACTGAAAATGCATGGCTTTAGAAACTTCTGGGT
AGTTTGAATTGCAGACAACCAGGCCCTATTCCTGTTTGTTTAACTAATGAGATTTGTAAACAGTACTTGG
TTTCCCCAGACTCTAGGACCTAGAGGAAGGAAATTTTGATTTAGCTCTTGGTAAAGCATTTAACACCTAA
TTTTTCTTTTTCATTGTTATTTTCTTGATCACAGAAAACAATACATGTTAATAGTAGAAAATACCCACAA
TTGAAAAGAATATAAAGCACTTACTAAAACTCTACCACCTAGGCTAGGTACGGTGGCTCACACCTGTAAT
CCCAGTACTTTGGGAGGCTGAGGTGGGTGGATCACCTGAGGTCAGGATTTCAAGACCATCCTGGCCAACA
TGGTGAAACCCCATATCTACTAAAACTACAAAAATTAGCTGGGCGTGGTGGCAGCCACCTGTAATCCCAG
CTGCTCGGGAGGCTGAGGCAGGAAAATCGCTTGAACCCAGGAGGAGGAGGTTGCAGTGAGCCGAGATCAT
GCCGCTGCACTCCATCCTGGGTGACAGAGCGAGACTCTTGTCTCAAAAAACAAACAAAAAAACTGCCACC
TAGAGATGGTGGTGGCCAACAGCTTAGTAAGAATTTCTCTTGCATTTTTCTATGCAAACACATTCAAATT
TTCATTTTTTAAAAAAGATGATAGAATCATACTGTATGAATTTAATTGCCTAATTAAGCTTGTGTATCAT
CGTTTCTCTTGTCATTAAATGCTGTACAGCTTTCTTTTAAATGGCTATCTGTGGTAGTGTTCCTTGAAGG
AAATACCTTAAAATTTTTTGTTTTTAAACTTTTATAATTGTGGGAATGTAATACAGTGAATGCATAGGAC
ACAAATGTTCAGCTTAAGGAATTATTATAAAGTGAACACCCTGTAACTGCCACTTAGATCACGACATCAA
ATATGGCCAGCAGCCAGGCGCACCTCCCCTACCCCCCACTGCCCCTTCCCAGTCACAGCCCCATTCTTCC
AGCTGAAGGTAACTTCTGTCTTGACTTTATGAAAGTTATTTTCTTGCTTTCCTTTAGATTACCTACCCGT
CTTTGCATTCCTAATGCTGTAGTTTAGTTGAGCTTATTTTGAACTTTTCACAAACGGAATCATCAACTAT
GCATTATTTTGTATCTGGCTTTCATTCACTATTTTGTTTTTAATATTCATCTATGTCGTTAGTAGCTATA
GTTCATTCATTTTTTGTTGCTGTATAATCATCCATTTATTGAATGTACTACAATTTGATTCTGCCTTTGG
CAGCCACTTTGATTGTTTATAATCCAGATTTCAAATGCTGCATCATGAACATGTGGGTTGTGATTTGTGT
AGATGTGCATGTACTTCTCTAAGGAATCCAAGCCTAGGAGGGACGTTGCTGGGTCAGAGGATGTTTGTAT
CTTCAACTGTACCAGATAAGGCCAAATTGTATTCTCAAATGCTTGGATCTGTTTTCAGTGTACTTGTTCT
ACTCCTAATTAACACTACTTGATATTGTCAAACTTTAATTGTAGCCATTCTGATGGATGTGGTAGTATCT
CATTGCGGTTTTAATTTGCATTATTTTGCATTTTCCTTATTGTTAGTGAGATTAAATACCCTTTTATATG
TTTTGGCTACTTCAGTATCCTCTTGTGCAGTGCCTAATTCACATCTTTTATTTTTCTTCTGAGTTGCCTT
TTGCCCATTGATTTATATTTGTTCCCCCTCTCTCCCCGCCCCCAAGAAGGAGTCTGGCTCTGTCACCCAG
GCTGGAGTACAGTGGCACAATCTTGGCTCACTAACCTCTGCCTCCTTCGTTCAAGTGATTCTCCTGCCTC
AGCCTCCCAAGTAGCTGGGACTGCAGGTGTGCGCCACCACGCCCAGCTAATTTTTGTATTTTTAGTAGAG
ACAGGGTTTCACCATGTTGACCAGGCTTGTCTTGAACCCCTGACCTCAAGTGATCCATCCGCCTTGGCCT
CCCAAAGTACTGGGATTACAGGTGCAAGCCACCATGCCCGGCCTGATTTATAAATGTAGTCTAAATACAA
GTTCTTTATTGGTTATGTAAGTTGCAGATATCTTTTTCAGCTCTGTAGGTAATAATGTTTTTTGATGAAT
AGGAGGTCTTAATTTTAATGTAGTTTAATTGATTTGTTTTCTCCTTTGTAAGAATCAATATTTTTCATGT
CCTGTGTAAATCTCGTATTGGGATAAAAGATACATATATATTTTTTAGGTTTTGGCTAATAGTTTTCCGT
TGGCAGCATGTGAACCTTTTTATGCCATTTTTACTTTCATCAAGGGTGTATGTATGATTTTCTCATACTC
TTGTCAGAACTGGAGATTTGGGGTAACTTTGCCAGTGTAATAGACTTTCTTAATTCATTAAGGAGTATTT
TATCTTAGTTTGCCCATGAGAGAAGAGAGATTAAATTTGTTTTATTATACCAAGAACATGGCAGTCAATA
AGCATAACTAAAAATATTATTTAGCTTTTGATTCTGAAGACCATGTATGGCCAGTAAGGAAAAGAGGTAA
AGATGTAGAAAACTGTTTTCTTTTTTTTCTTTCTTTTTTTTTTTTTTTTTTTTTTTTGAGATGGAGTCTT
GCTCTGTCGCCAGGCTGGAGTGCAGTGGCGTAATCTCAGCTCACTGCATCCTCCACCTCCTGGGTTCAAG
TGATTCCCCTGCCTTAGCCTCCCGAGTAGCTGGTACTACAGGCGCGTGCCACCACACCTGGCTAATTTTT
TGTATTTTAGTAGAAACAGGGTTTCACCGTGTTGGCCAGGATGGTCTCAATCTCCTGACCTCGTGATCCA
CCTGCCTCAGCCTCCCAAAGTGCTGGGATTACAGGTGTGAGTGAGCCACTGCACCTGGCCTCTTTTTTAT
TAATTTAAGATACATGATTTTGGCTAAAACTAATGAAAATAAATTGATGAAACTTCAATGTAGTGGATCA
AGAGACTCATGAAATTGATGAGTTAGATTGTTGTGGGCACCTGTTTTAAATTTATGGTGCCTTTTTAGCA
GAATCATGACTTTATCAACTCTTTTTCACATACTTTTATTCAAAGAAGACATTCTCCTCCTCTGCCCCAA
CTTATCTTCTTCTTGTCTCCAACTGCCAGTATTCTCATCCACACTCTTTGTAGTTTTGAGGTGTGAGATA
ACTCCCAAGTTCTTCTTGGGAATGGGTAGAGTTTAATTCTGAGTCTACATGTCGTGAGTCTATTGGAAGA
AGCTTTTCTTAGGAGGTGAAATTTGGGATGGAGTTCTCCTCTACACTCTAGCCCTGTAGCCACAATCTTT
GGTTTAGATCTTTGGCATTTTTTGTTGGACATCTTTCATAGTGTCTACCTCTTCTTCCTGCCTCTAGGCT
TCCCGCGGTTGCCAGATGAAATTCCGAAAACTCAAATCATTCCTGTAGTTAATATTCTTCAGTGACTCCG
GAACAATCAAATTCTTGATCCCTCCACATGGGTCTTACCATCTGCCCTCGTGCCCTCTCTAGCTTCCTGC
ATATTGAGTTCCCCACAGCCCTAGTTTCCCTGAGTTTGGAATATTCTTTCACATTTGCTCATCCCACTAT
CTAGAAACTTCCTACAATCTCTCTACTGCCAACCTGTCTGTCTAGTTGCTAATTTATTTTAAAACCCAGC
TTAAGCAACACATCCTTTTGGATGTGTTACTTCCCTGATCGTTATTCACCTCCATGCCGTCTTTCGCTCA
ACTCATTGAAGTTAGGTGCCCCTCTTCTGGGCTGCCACGGCTCTCCATGTTCACCTATGTACTGTACTTT
GTATTGTAATCAGTTTAATTGACCCCCCTACTTATTTGAGCTCCTTGAGAGCAGGGCCTTGATTCCTGCT
CCTTAGCACTCCACCCTCCCCAGAACTAACATGGTATAGCTTATAGGAGGCACCCTATAAATGTTTAATG
AATGTTTGAATGCAAAAGTGTATCTAACTTTCTTAATGATATATTTATGAAAAGAACTTGCTGAGAAAAA
GGTCAGTTAGAAAGGGTTAGGAACTTGTCAGTATTCTAAATCCCTCTTTTTCAAAAGCATACACATTTAG
GAATGACTGCCTTGCTTTAAGAATTTGTAGGGTTTTGTTCACCTGAGTTGTACCTTGCTGTCACAGAAGG
ATGCCCTGCAGTGGTCTCGCCATCCAGCTACAGTGGAGGGAGAGGAGCCAGAGGACACGGCTGATGTGGA
GAGCTGTGGGTCTAATGAAGCCAGCACTGTGAGTGGTGAAAACGATGGTAAGGACCCTTTAATGGATGGG
TGAGGGAGCCACAGCAGGCACTAGGGACTAACCTTTATTTTCCTCTCTTCCAGTATCTCTTGATGAAACA
TCTTCGAACGCATCCTGTTCTACAGAATCTCAGAGTCGACCTCTTTCCAATCCCAGGGACAGCTACAGAG
CTTCCTCACAGGTAAGGAAGAGGTAGAGCCTAGCCTGGGAGGATGAATGATGACAGGTATAAGGCAAGAT
CCCTCCTTCTCCTGTAGTGAGCTGTGAACATGTTCAGAGAGTGAAGAATAGAAGTCTGGTCTCCAGCTGT
TAGTAGCATTTAACAGGGGGCCGCAGGAAAAGTGAACAGTAGGTTTGAGCAGAGTAAGTTTTTTCTCTTT
CTTTACCTTGGCTGAGTGATTTTGTTCATTCTTTTTTTTTTTTTTTTTTTTTTTTTTTGAGATGGAGTCG
CGCCTGTCGCTCAGGCTGGAGTGCAGTGGCATGATCTCCGCTTACTGCAACCTCTGCCTCTGGGGTTCAA
GCAATTCTCCTGCCTCAGCCTCCCGAGTAGCTGGGATTACAGGCACGTACCACCGTGCCTGGCTAATATT
TGTATTTTTAGTAGAGATAGGGTTTTGCCATGTTGGCCAGGCTAGTCTCGAATTCCTGACCTCAAGTGGT
CTGTCCACCTCAGCCTCCCAAAGTGCTGGGATTACAGGTGTGAGTCACCATGCCTAGCCTGATTCTGTTC
ATTCTTGTAATATACACGTGAACATTCAGGGAGAAGTGTGGTTAGTGCAGAGTAGAGAGAACAGGAGATT
GGGAGTGAGCAGTTTGAATGATAACCCTGCACTTACTAGCCTTGAGACTTGGGGAAAATTCCTTACCTGT
AAAATGGGGATAACAGTACATTTTTGTAGCTTGGAATGATGCTTGGCACAGTGACCAGCACGTAGCTCAG
TAACATTAACCAGTGCTCTTGTCAGCATTATTTGACAGATCTGGTTGAAGACGAACTTCATTTTACAAGA
GCGTGAGTAGAGATAGTGTCGCCAGGGAATGCTTTTGTGGCTCTGCAGTTGACTTGGGCTCTCTTTTGTT
CTCTCTTGGAACGCAGGCGAACAAACAAAAGAAAAAGACTGGGGTGATGCTGCCTCGAGTTGTCCTGACT
CCTCTGAAGGTAAACGGGGCCCACGTGGAATCTGCATCAGGTATGTGTAAACTCATGGTTGTGATGCTTT
TTCCTCAGGTCCTGGGGACCTGGCCTCCCTCTGTCAGCAGTTTCTGACCTAATAGTGCGATACTAGGAGA
GCTGTCGGGCCACAGGCAAGAGCCCTGCCAATGGATGAGGCCTGCCATAGGTTCTAGTGCTGGGCTCTGC
TGTGTGCCTTCCTCTTTGTCTCCCAGGGCAGTCGTGAGGATCCAACGGAGGATTTGATTATGCCAGTGCT
TTGTAAAAGGTGTAGTGCTATACAAATAAAGATGGCAGTTTGGCACCTGTAAGGTGATATTTTAAAGCCA
GACCATGAAGTGGTGGTTTCTCTCAGCCTAAGGCTGGGAGATGGAAGCATCCCAGTATTGCTGGCAGCAT
CTCAGGGAGAGCTGGGAGAAATGAGCTTGTCTGAGAGCCATGGGCGCGGCTTGGTGATACTTTTGACCAG
TGGAATGCTGTGCCTTCAGGGTTCTCGGGCTGCCACGCCGATGGCGAGAGCGGCAGCCCGTCCAGCAGCA
GCAGCGGCTCTCTGGCCCTGGGCAGCGCTGCTATTCGTGGCCAGGCCGAGGTCACCCAGGACCCTGCCCC
GCTCCTGAGAGGCTTCCGGAAGCCAGCCACAGGTGAGTGGCGTGGCACTTATTTCTCTGCCTGTAAAGGG
GGCCCCTGCAGGCCTAGTGAGAAGATGATGTCTTCTATTCCAATTCCTTTACCAGTTTATTTTTACTTCT
TGGGTGGCCCTCTATTTTTTGTTTATTTTTACTTTGTAATTTGCTTATTTCATTTGTAGCTCTCTGCTAA
ACTGTGAGAACTCCTTTCAGGCAGAGATGATGCCATACTGTTTCTCGTATGTCCCTGTAGGCCTAGTCTA
TGCCTGCCTTGTACTGCATAGTAAGTGTTTGAGTGAGTGAAAACTGCTTCTGAGCTTCTCAGTGAAGAAT
TCCAACACCCCTTTTCTCTCTTCGGTAGCATGTTGCCACATTTGTTCCCAACATCTGTAGTCTGTATCAG
ACGCTCCCTTTTTTACAACCTAGTCAGTATGTTGAGTGGAAGCATGTGTTGAATTTTGGGGATGCAAACA
AATTCAGTAGTGCTATTATCTATACCTCCCACCCCTCACCCCACCCCACCCCCATTAGGGTTATTTGCAA
TATTAGTATGCTCTTTGCTATTTCAGGATTTAATTCTTAATGACCCACATGCTGGTTTTTTTTGTAGATG
AAACCATTAAGAAGACAAGCTTTTGCTTCTGAGAATTAGAGTAAACCTTTGCCCATGTGAGAACTAGGTT
TGCTGTCTTGCTGCTTTTCCAGCTCATCAGCAGTGCTCAATCTAAGGTCTCACAGGTCTCTAGTCCTAGT
GAGTTTTTCAGATAAAAGTAACATTTGCTGAGGCTTCTGTGAGAGGTATTAGCATCTCAGTTGGCTTTGA
ACAGTCCTTAGTACATTTCTGCATGGAGATCATCCCTCTCCAGCCCATTCACAGAAGGCTTCTGCAGATT
TTTCTATAGTATACTAACTAGATTTACTTTTAGATAATTCTCATGATTTCTCCAGTTGAGCTTTGTGGAG
CCTGTTCTCCAGCTTTGGAGGGAAGCTCAGAACCTGCCCCTTGCCTGATTTTCTGTGGGAAGTGTCCTTT
TCAGGCTGTGACACAGCACGGTACAGAGTTTTGTCTGAGGAGATGCAACCCCAAGTGTTCTCCTGGGTTT
GATGGCAATGACAGTAACCTGGAAAGAATATACAGGTTAGGATGGACTGGACAATTGAGCAGATTGTGTG
CAGATAACTCCTGGGTAGCTTGGGTGCTGTCACCAAAGTTTTCCCATCAGTTGGCATTTGTTTTTTCTTT
TAAAAAGCTGAAATCTATACCTTGCTTCAAAAATCATAGGTCAAATGAAGCGCAACAGAGGGGAAGAAAT
AGATTTTGAGACACCTGGGTCCATTCTTGTCAACACCAACCTCCGTGCCCTGATCAACTCTCGGACCTTC
CATGCCTTACCATCACACTTCCAGCAGCAGCTCCTCTTCCTCCTGCCTGAAGTAGACAGACAGGTGCACA
TGGGCAGCCTCCCCTTTGCCTCTCTCTGGGTGGGCTTCTGTTCTCTTTTAAGTTTATTTATTAGGATTTT
TTTCCCCCTTGATCCTTCTAGGTGGGGACGGATGGCCTGTTGCGTCTCAGCAGCAGTGCACTAAATAACG
AGTTTTTTACCCATGCGGCTCAGAGCTGGCGGGAGCGCCTGGCTGATGGTATGTAGACTTGGTCATCTCG
GACGGCTTGCGACGCACCTGTCGTGTGGTGTTGCATGTCTCCTGGTATTTAAAACCCAAGCTCGCTCTTC
TCAAAGGGTTATTTAGTATAAGACAGGCTTTCCTCATTACTAATAAATGTGGTGGGTATTAATATGCCAC
ACAGCTCTGAAGAAATAACAGGGTGTTTAGGTTTAGACTTTCACTATATTGTATGTATGTACTCATTCAT
CATCTCCTTTTTATCATTAAAACTATAAACACCTATTCCAGTAATCATGGCATGGTCTGGAAGGGTGGAG
GAAAGTACACTGTGGAGTTGAGAAGCTCCACCATGCTCTGTCTTTTTAGGCATTGTCACTTGGAGTGATT
GTTAGCTTCTATTAATTTAATTTTGTGGGGAAAGTGTTGAGAGGGCTCTAAGAACACCCCTAGGTTTGAT
GATTTGCTAGCAGGACTCAGCATATAGTTATATGTACAGGGCTGCGGTTTATTACAGCAGAAGAATACAA
AGTAAAATCAGCAGATGGAGAGGCACATGGGGTGGGGTCCAGGGGAAACCATGTGTTGGCTTCCAAGGTC
CTCTCTCAGTGGTGTCACACAGGACACATTTAATTCCCCCAGTAACAAGTTATGATAACATGTATGAAAT
GTCTACTAGGGAAACCTGTTAAAGACTCGGTGCCCAGGGTTTTTATCAAGGGCTGATCATGTAGGAAGCC
ACTGCTTGGCACATACCCAGCTTCCAGATTCCCAAAAGGAAGGCAGGTGTTCAGCATAAGCCGTATTGTT
TGCATAAACAATTTAGGCACAGTGAACAACTCATATCAGTTAGGGTGGTTGGGAACCCTCCTGAAATCTA
AGTTCCCAGATGTCAGCCAAGGGCTGTTCTTTTAAGTAGGCCTTTCTAAGAGTAAACAATCAGGCCTACC
TACTATGTTAACTTTTTATCTACACAGGAAATATGAATGGTTCTTTTGTTTGTTGTTTTGCTTTTTCGAT
CAAGGAGTTGCTTGGTCTCACTTTAAGGAAGAGTGAATTTCCCTTATAGTAGATGTGTTAGCTCTGTCCC
TATAAGAGCATGATGTGAGAGAGCCTTTAGAAGAGACGTGTTGTTTTAAAACTGGGAGATTCAGCTGTCC
ATAAGACAGACATTAATATCCCGAATGCACTTACTAGAAGAGGTTTATTTCTCCCTAGGTGAATTTACTC
ATGAGATGCAAGTCAGGATACGACAGGAAATGGAGAAGGAAAAGAAGGTGGAACAATGGAAAGAAAAGTT
CTTTGAAGACTACTATGGACAGAAGTAAGGCAGTTGGAGCTATGAGTCCTGGTCTGGGGTTTTGAGGGGA
TAGAGGTAGATGGTCTCAAAATAGCATATACTTATGTGTTGGACAAGAATGTGGAGATTGCAGTGACACT
TGGGTCATTTATCTTAATGCCATTCATAGTGAAGCTAACAGAAGTTTTTCCATGGTTAGATTGTGCACCA
CACAGATTTATTTTGTTCTGAGATATCTGTGTTTCTGGGTCCATATTATTCATAGAAATAAGAGACATGT
CCACTCTGGCCTGAAACTGATGGCTGTGATTTTGATTTGCAGGCTGGGTTTGACCAAAGAAGAGTCATTG
CAGCAGAACGTGGGCCAGGAGGAGGCTGAAATCAAAAGTGGCTTGTGTGTCCCAGGAGAATCAGTGCGTA
TACAGCGTGGTCCAGCCACCCGACAGCGAGATGGGCATTTTAAGAAACGCTCTCGGCCAGATCTCCGAAC
CAGAGCCAGAAGGAATCTGTACAAAAAACAGGAGTCAGAACAAGCAGGGGTTGCTAAGGATGCAAAATCT
GTGGCCTCAGATGTTCCCCTCTACAAGGATGGGGAGGCTAAGACTGACCCAGCAGGGCTGAGCAGTCCCC
ATCTGCCAGGCACATCCTCTGCAGCACCCGACCTGGAGGGTCCCGAATTCCCAGTTGAGTCTGTGGCTTC
TCGGATCCAGGCTGAGCCAGACAACTTGGCACGTGCCTCTGCATCTCCAGACAGAATTCCTAGCCTGCCT
CAGGAAACTGTGGATCAGGAACCCAAGGATCAGAAGAGGAAATCCTTTGAGCAGGCGGCCTCTGCATCCT
TTCCCGAAAAGAAGCCCCGGCTTGAAGATCGTCAGTCCTTTCGTAACACAATTGAAAGTGTTCACACCGA
AAAGCCACAGCCCACTAAAGAGGAGCCCAAAGTCCCGCCCATCCGGGTAGGAGACTGTTTGATTCCTGGC
TGCCCTGGAGCCAGGTTTTCTTTGAGGGTCATGAGATTATAGAGCCCTTAACTCTGGGGCCCTGAAGTTA
ATTATGTGGGAATGTAGAGCTTTTTATGAGAGGTTTGCTTGAGACAGCCTTCAAGTTTAGTGAGATAGGT
TCTTTTCTTCCTCACTTTTTTGTTCACTCTGTTGAAGTTATCTTGAGAGGTCAAAATCCTTTGGGGTTTT
AAAATCTTTTTAAGATAGCATTAGATTCTTTGCTATAGTGACTCACACAGTCCCACCAGAAATTAAATTT
AGAAGTGTGGCATATAACAGCCCTTGAGCAGAATCTTCTCTGAATGGTGTGTTATGGCGCCATTTTACTA
TGATGATTTCATTGTTTTAAGGAAATTATTTGATTCTGTATGCCATGACCCTTAAGCTACTAGAATCCTA
GTTTTGCTTTACAGTCCCTAGGTCAGATCACCCAGTCAGTTAAAACTATTTTCTAATTCTTTTTTTGCAG
ATTCAACTTTCACGTATCAAACCACCCTGGGTGGTTAAAGGTCAGCCCACTTACCAGATATGCCCCCGGA
TCATCCCCACCACGGAGTCCTCCTGCCGGGGTTGGACTGGCGCCAGGACCCTCGCAGACATTAAAGCCCG
TGCTCTGCAGGTCCGAGGGGCGAGAGGTCACCACTGCCATAGAGAGGCGGCCACCACTGCCATCGGAGGG
GGGGGTGGCCCGGGTGGAGGTGGCGGCGGGGCCACCGATGAGGGAGGTGGCAGAGGCAGCAGCAGTGGTG
ATGGTGGTGAGGCCTGTGGCCACCCTGAGCCCAGGGGAGGCCCGAGCACCCCTGGAAAGTGTACGTCAGA
TCTACAGCGAACACAACTACTGCCGCCTTATCCTCTAAATGGGGAGCATACCCAGGCCGGAACTGCCATG
TCCAGAGCTAGGAGAGAGGACCTGCCTTCTCTGAGAAAGGAGGAAAGCTGCCTACTACAGAGGGCTACAG
TTGGACTCACAGATGGGCTAGGAGATGCCTCCCAACTCCCCGTTGCTCCCACTGGGGACCAGCCATGCCA
GGCCTTGCCCCTACTGTCCTCCCAAACCTCAGTAGCTGAGAGATTAGTGGAGCAGCCTCAGTTGCATCCG
GATGTTAGAACTGAATGTGAGTCTGGCACCACTTCCTGGGAAAGTGATGATGAGGAGCAAGGACCCACCG
TTCCTGCAGACAATGGTCCCATTCCGTCTCTAGTGGGAGATGATACATTAGAGAAAGGAACTGGCCAAGC
TCTTGACAGTCATCCCACTATGAAGGATCCTGTAAATGTGACCCCCAGTTCCACACCTGAATCCTCACCG
ACTGATTGCCTGCAGAACAGAGCATTTGATGACGAATTAGGGCTTGGTGGCTCATGCCCTCCTATGAGGG
AAAGTGATACTAGACAAGAAAACTTGAAAACCAAGGCTCTCGTTTCTAACAGTTCTTTGCATTGGATACC
CATCCCATCGAATGATGAGGTAGTGAAACAGCCCAAACCAGAATCCAGAGAACACATACCATCTGTTGAG
CCCCAGGTTGGAGAGGAGTGGGAGAAAGCTGCTCCCACCCCTCCTGCATTGCCTGGGGATTTGACAGCTG
AGGAGGGTCTAGATCCTCTTGACAGCCTTACTTCACTCTGGACTGTGCCATCTCGAGGAGGCAGTGACAG
CAATGGCAGTTACTGTCAACAGGTGGACATTGAAAAGCTGAAAATCAACGGAGACTCTGAAGCACTGAGT
CCTCACGGTGAGTCCACGGATACAGCCTCTGACTTTGAAGGTCACCTCACGGAGGACAGCAGTGAGGCTG
ACACTAGAGAAGCTGCAGTGACAAAGGGATCTTCGGTGGACAAGGATGAGAAACCCAATTGGAACCAATC
TGCCCCACTGTCCAAGGTGAATGGTGACATGCGTCTGGTTACAAGGACAGATGGGATGGTTGCTCCTCAG
AGCTGGGTGTCTCGAGTATGTGCGGTCCGCCAAAAGATCCCAGATTCCCTACTGCTGGCCAGTACTGAGT
ACCAGCCAAGAGCCGTGTGCCTGTCCATGCCTGGGTCCTCAGTGGAGGCCACTAACCCACTTGTGATGCA
GTTGCTGCAGGGTAGCTTGCCCCTAGAGAAGGTTCTTCCACCAGCCCACGATGACAGCATGTCAGAATCC
CCACAAGTACCACTTACAAAAGACCAGAGCCATGGCTCGCTACGCATGGGATCTTTACATGGTCTTGGAA
AAAACAGTGGCATGGTTGATGGAAGCAGCCCCAGTTCTTTAAGGGCTTTGAAGGAGCCTCTTCTGCCAGA
TAGCTGTGAAACAGGCACTGGTCTTGCCAGGATTGAGGCCACCCAGGCTCCTGGAGCACCCCAAAAGAAT
TGCAAGGCAGTCCCAAGTTTTGACTCCCTCCATCCAGTGACAAATCCCATTACATCCTCTAGGAAACTGG
AAGAAATGGATTCCAAAGAGCAGTTCTCTTCCTTTAGTTGTGAAGATCAGAAGGAAGTCCGTGCTATGTC
ACAGGACAGTAATTCAAATGCTGCTCCAGGAAAGAGCCCAGGAGATCTTACTACCTCGAGAACACCTCGT
TTCTCATCTCCAAATGTGATCTCCTTTGGTCCAGAGCAGACAGGTCGGGCCCTGGGTGATCAGAGCAATG
TTACAGGCCAAGGGAAGAAGCTTTTTGGCTCTGGGAATGTGGCTGCAACCCTTCAGCGCCCCAGGCCTGC
GGACCCGATGCCTCTTCCTGCTGAGATCCCTCCAGTTTTTCCCAGTGGGAAGTTGGGACCAAGCACAAAC
TCCATGTCTGGTGGGGTACAGACTCCAAGGGAAGACTGGGCTCCAAAGCCACATGCCTTTGTTGGCAGCG
TCAAGAATGAGAAGACTTTTGTGGGGGGTCCTCTTAAGGCAAATGCCGAGAACAGGAAAGCTACTGGGCA
TAGTCCCCTGGAACTGGTGGGTCACTTGGAAGGGATGCCCTTTGTCATGGACTTGCCCTTCTGGAAATTA
CCCCGAGAGCCAGGGAAGGGGCTCAGTGAGCCTCTGGAGCCTTCTTCTCTCCCCTCCCAACTCAGCATCA
AGCAGGCATTTTATGGGAAGCTTTCTAAACTCCAACTGAGTTCCACCAGCTTTAATTATTCCTCTAGCTC
TCCCACCTTTCCCAAAGGCCTTGCTGGAAGTGTGGTGCAGCTGAGCCACAAAGCAAACTTTGGTGCGAGC
CACAGTGCATCACTTTCCTTGCAAATGTTCACTGACAGCAGCACGGTGGAAAGCATCTCGCTCCAGTGTG
CGTGCAGCCTGAAAGCCATGATCATGTGCCAAGGCTGCGGTGCGTTCTGTCACGATGACTGTATTGGACC
CTCAAAGCTCTGTGTATTGTGCCTTGTGGTGAGATAATAAATTATGGCCATGGGAAACATTGTATATTTA
GTGTGTGTATTTTGATAATGATTGATCTTAAATCTGTATACAGAATATCATTGATATAATACTCTTTAGG
CAGGAGCACTCTTGCCTTCCCCCAAAATTTACACTGCTAAAGCCCTCTGTCACTTGGCGACCCTTCTGGT
CTTGCTGGAGGGGTTTCCTGGGTATAACCCATTGGGCTGCCCAAGGCCAGCCAGCCTGAGCTCTCCTGCA
AGACAGAGCCTGATGTGGCACGGAGTGGGGTTGCGGGGGGTGGGGGGACTGCCTGACTCCCAGAGGGACT
TGAAACTGAAGCAAGAAGGTTGCATTCTCCACCAAGGGAGTTAACCTACCTGAACTAAGTAGAAATGCCA
GTCTTCCACTACCCCCTCCCTGCCATCTTTTCTTCTGCTACTTTGGGGAGTTGATGGCCAGGAAAGAAGC
CAGCACAGGGTTAAAGTAACTCCTGGCATTGCCCACCAGGGGGCTGGTGCACCTGCTGACCTCAGGGTCA
CAGTTGAGTCATTTGCCAGTTGACGGAGCAAGTTTGACCTTGGTTCTGTTGCTGAAGCAAATTTGGAACT
TTTCTGTCTCAGTGTGATCCACTAACCCACAGGATCATTTGGAACCTTGAATAGCTCTGCTTGGACAATG
GGGTTGGGGAATAGGGTTGTCTTTCCTATGAAAATGCCATCTGTAGACCTTGTGAGTCAGCCGTCCAGAT
GTTTGCAGGTGAATTCCTCTGCTTGACATCCTCCCTGTCACTTTGGACCCTATGGGAGTGGGCATCTCCA
CGCACCTGTGTATGTGAAAGTCATTTTACATTTCAAAGCAGTGTGTGTTTCTTATTTTTATATTTTTAAC
TCTTTATTCTTGGATGTATAAAGTGAACTTTTTGGCTTCTGTAAGTATGCTCTATGCACCTCTAATGTTT
TATCATGTATTTATATGTTGTACACAGTACTGGCTGATTCTGTAAATGGATGTATTGTACAGAGAACATG
AACGTCTCTTCCTAATTTTACATCTTCAGCATCATTGCATTAAAGTGGTGTAATCTCCTTCTCTACATCT
GTTGTCAGAGCCACTGAGTGCTGTGCTGCTCGACGTGAGGGTGAAATGATTGACTTGTGACCTGCCAGGT
TGCCCGATGCCCTGTTGGGTCACCGGCTGGACCTGCTGCAGCCTGCAGAGCCACAGTCAGCCTGCCCACA
TGCCACCGAGCAAACGCATCTTGCTTTTCACATCTCTCCTCCTACAGCCTTAATGGCTGCTTGCTGCCAT
ATGTGACAAATCACCACCACCAGTGTTAAGTGCTTCTGGATTCATGGGTGAGTTCCCTGGGCAGCCCCCA
GGAAGGCCTTCCAGATCTGGCTCCAGGGTCACCACCTGTCACAGCAATACCTGGGACCATGCTCTCCTGG
GACTGTGAGGCTCCTTTTGACGTACTTTTGACATCAGGCAGGTTTGGGAAGAAACAAAGCCATGCCTGCT
CCTGCCTCTCTCCCAACATGTTTCCAGCAAGTAGATGCCCCTGTGTGTGTTTTCCCTTGCCTTGTTTCCT
GCCTTATATCTTGTATTTCGACTTATTACAGAGTTGAGGGTTCTTGCTTAATTTAGATCAAGTATAAAAT
TTGTATGACTTCAAGTCTCATTTTATCTGAAAGGTTTTTTTCTCATTTAATCTGATGTGGCATTTTCGTC
ATCTGAAGCATGAGTGACAAGTTGGGAATGATGTGGTGATTTAGAATGCAGTATTGGCCAAGTCCAAGTT
GTCAACTTAAGCGTCTGTTTACCAAAGACCGGGAACAGGGGCCCAAACATGTCCAGTCCTCTTCTTCCCT
CTGCTGGAACCTTTGGGGACACTCAAGGGTACAGTTTGACACTGATCTGGTCCATGAGGCTGCCCAGAGA
AAGCACTGCTTCTGTATGTCTCTTGTGGTATTGGAACAATAAACCCGTACAACCTGCA
SEQ ID NO: 24 (NM_015338.6, AXSL1 isoform 1, mRNA):
ACACACACTCACACACACCCACGGCAGACACGCACGCACCCGGGCGCCGAAGGGAAAGCCGCGTCTCGCC
CTCCCGCCCCGCCGTCGGTCCTGTCTCAGTCCCTCAGCAGAGCGGGAAAGCGGAGGCCGGAGCCGTGACC
TCTGACCCCGTGGTTATGCGGAGCCGCCGCATTCCTTAGCGATCGCGGGGCAGCCGCCGCTGCCGCCGTG
GGCGACTGACGCAGCGCGGGCGCGTGGAGCCGCCGCCGCCCCTCCCCCACCGCCGCTCTCGCGCCAGCCG
GTCCCCGCGTGCCCGCCCCTTCTCCCCGGCCGCACCCGAGACCTCGCGCGCCGCCGCTGCCACGCGCCCC
CCCCACCGCCGCCGCCGCCCCAGCCCCGCGCCACCGCCCCAGCCCGCCCAGCCCGGAGGTCCCGCGTGGA
GCTGCCGCCGCCGCCGGGGAGAAGGATGAAGGACAAACAGAAGAAGAAGAAGGAGCGCACGTGGGCCGAG
GCCGCGCGCCTGGTATTAGAAAACTACTCGGATGCTCCAATGACACCAAAACAGATTCTGCAGGTCATAG
AGGCAGAAGGACTAAAGGAAATGAGAAGTGGGACTTCCCCTCTCGCATGCCTCAATGCTATGCTACATTC
CAATTCAAGAGGAGGAGAGGGGTTGTTTTATAAACTGCCTGGCCGAATCAGCCTTTTCACGCTCAAGAAG
GATGCCCTGCAGTGGTCTCGCCATCCAGCTACAGTGGAGGGAGAGGAGCCAGAGGACACGGCTGATGTGG
AGAGCTGTGGGTCTAATGAAGCCAGCACTGTGAGTGGTGAAAACGATGTATCTCTTGATGAAACATCTTC
GAACGCATCCTGTTCTACAGAATCTCAGAGTCGACCTCTTTCCAATCCCAGGGACAGCTACAGAGCTTCC
TCACAGGCGAACAAACAAAAGAAAAAGACTGGGGTGATGCTGCCTCGAGTTGTCCTGACTCCTCTGAAGG
TAAACGGGGCCCACGTGGAATCTGCATCAGGGTTCTCGGGCTGCCACGCCGATGGCGAGAGCGGCAGCCC
GTCCAGCAGCAGCAGCGGCTCTCTGGCCCTGGGCAGCGCTGCTATTCGTGGCCAGGCCGAGGTCACCCAG
GACCCTGCCCCGCTCCTGAGAGGCTTCCGGAAGCCAGCCACAGGTCAAATGAAGCGCAACAGAGGGGAAG
AAATAGATTTTGAGACACCTGGGTCCATTCTTGTCAACACCAACCTCCGTGCCCTGATCAACTCTCGGAC
CTTCCATGCCTTACCATCACACTTCCAGCAGCAGCTCCTCTTCCTCCTGCCTGAAGTAGACAGACAGGTG
GGGACGGATGGCCTGTTGCGTCTCAGCAGCAGTGCACTAAATAACGAGTTTTTTACCCATGCGGCTCAGA
GCTGGCGGGAGCGCCTGGCTGATGGTGAATTTACTCATGAGATGCAAGTCAGGATACGACAGGAAATGGA
GAAGGAAAAGAAGGTGGAACAATGGAAAGAAAAGTTCTTTGAAGACTACTATGGACAGAAGCTGGGTTTG
ACCAAAGAAGAGTCATTGCAGCAGAACGTGGGCCAGGAGGAGGCTGAAATCAAAAGTGGCTTGTGTGTCC
CAGGAGAATCAGTGCGTATACAGCGTGGTCCAGCCACCCGACAGCGAGATGGGCATTTTAAGAAACGCTC
TCGGCCAGATCTCCGAACCAGAGCCAGAAGGAATCTGTACAAAAAACAGGAGTCAGAACAAGCAGGGGTT
GCTAAGGATGCAAAATCTGTGGCCTCAGATGTTCCCCTCTACAAGGATGGGGAGGCTAAGACTGACCCAG
CAGGGCTGAGCAGTCCCCATCTGCCAGGCACATCCTCTGCAGCACCCGACCTGGAGGGTCCCGAATTCCC
AGTTGAGTCTGTGGCTTCTCGGATCCAGGCTGAGCCAGACAACTTGGCACGTGCCTCTGCATCTCCAGAC
AGAATTCCTAGCCTGCCTCAGGAAACTGTGGATCAGGAACCCAAGGATCAGAAGAGGAAATCCTTTGAGC
AGGCGGCCTCTGCATCCTTTCCCGAAAAGAAGCCCCGGCTTGAAGATCGTCAGTCCTTTCGTAACACAAT
TGAAAGTGTTCACACCGAAAAGCCACAGCCCACTAAAGAGGAGCCCAAAGTCCCGCCCATCCGGATTCAA
CTTTCACGTATCAAACCACCCTGGGTGGTTAAAGGTCAGCCCACTTACCAGATATGCCCCCGGATCATCC
CCACCACGGAGTCCTCCTGCCGGGGTTGGACTGGCGCCAGGACCCTCGCAGACATTAAAGCCCGTGCTCT
GCAGGTCCGAGGGGCGAGAGGTCACCACTGCCATAGAGAGGCGGCCACCACTGCCATCGGAGGGGGGGGT
GGCCCGGGTGGAGGTGGCGGCGGGGCCACCGATGAGGGAGGTGGCAGAGGCAGCAGCAGTGGTGATGGTG
GTGAGGCCTGTGGCCACCCTGAGCCCAGGGGAGGCCCGAGCACCCCTGGAAAGTGTACGTCAGATCTACA
GCGAACACAACTACTGCCGCCTTATCCTCTAAATGGGGAGCATACCCAGGCCGGAACTGCCATGTCCAGA
GCTAGGAGAGAGGACCTGCCTTCTCTGAGAAAGGAGGAAAGCTGCCTACTACAGAGGGCTACAGTTGGAC
TCACAGATGGGCTAGGAGATGCCTCCCAACTCCCCGTTGCTCCCACTGGGGACCAGCCATGCCAGGCCTT
GCCCCTACTGTCCTCCCAAACCTCAGTAGCTGAGAGATTAGTGGAGCAGCCTCAGTTGCATCCGGATGTT
AGAACTGAATGTGAGTCTGGCACCACTTCCTGGGAAAGTGATGATGAGGAGCAAGGACCCACCGTTCCTG
CAGACAATGGTCCCATTCCGTCTCTAGTGGGAGATGATACATTAGAGAAAGGAACTGGCCAAGCTCTTGA
CAGTCATCCCACTATGAAGGATCCTGTAAATGTGACCCCCAGTTCCACACCTGAATCCTCACCGACTGAT
TGCCTGCAGAACAGAGCATTTGATGACGAATTAGGGCTTGGTGGCTCATGCCCTCCTATGAGGGAAAGTG
ATACTAGACAAGAAAACTTGAAAACCAAGGCTCTCGTTTCTAACAGTTCTTTGCATTGGATACCCATCCC
ATCGAATGATGAGGTAGTGAAACAGCCCAAACCAGAATCCAGAGAACACATACCATCTGTTGAGCCCCAG
GTTGGAGAGGAGTGGGAGAAAGCTGCTCCCACCCCTCCTGCATTGCCTGGGGATTTGACAGCTGAGGAGG
GTCTAGATCCTCTTGACAGCCTTACTTCACTCTGGACTGTGCCATCTCGAGGAGGCAGTGACAGCAATGG
CAGTTACTGTCAACAGGTGGACATTGAAAAGCTGAAAATCAACGGAGACTCTGAAGCACTGAGTCCTCAC
GGTGAGTCCACGGATACAGCCTCTGACTTTGAAGGTCACCTCACGGAGGACAGCAGTGAGGCTGACACTA
GAGAAGCTGCAGTGACAAAGGGATCTTCGGTGGACAAGGATGAGAAACCCAATTGGAACCAATCTGCCCC
ACTGTCCAAGGTGAATGGTGACATGCGTCTGGTTACAAGGACAGATGGGATGGTTGCTCCTCAGAGCTGG
GTGTCTCGAGTATGTGCGGTCCGCCAAAAGATCCCAGATTCCCTACTGCTGGCCAGTACTGAGTACCAGC
CAAGAGCCGTGTGCCTGTCCATGCCTGGGTCCTCAGTGGAGGCCACTAACCCACTTGTGATGCAGTTGCT
GCAGGGTAGCTTGCCCCTAGAGAAGGTTCTTCCACCAGCCCACGATGACAGCATGTCAGAATCCCCACAA
GTACCACTTACAAAAGACCAGAGCCATGGCTCGCTACGCATGGGATCTTTACATGGTCTTGGAAAAAACA
GTGGCATGGTTGATGGAAGCAGCCCCAGTTCTTTAAGGGCTTTGAAGGAGCCTCTTCTGCCAGATAGCTG
TGAAACAGGCACTGGTCTTGCCAGGATTGAGGCCACCCAGGCTCCTGGAGCACCCCAAAAGAATTGCAAG
GCAGTCCCAAGTTTTGACTCCCTCCATCCAGTGACAAATCCCATTACATCCTCTAGGAAACTGGAAGAAA
TGGATTCCAAAGAGCAGTTCTCTTCCTTTAGTTGTGAAGATCAGAAGGAAGTCCGTGCTATGTCACAGGA
CAGTAATTCAAATGCTGCTCCAGGAAAGAGCCCAGGAGATCTTACTACCTCGAGAACACCTCGTTTCTCA
TCTCCAAATGTGATCTCCTTTGGTCCAGAGCAGACAGGTCGGGCCCTGGGTGATCAGAGCAATGTTACAG
GCCAAGGGAAGAAGCTTTTTGGCTCTGGGAATGTGGCTGCAACCCTTCAGCGCCCCAGGCCTGCGGACCC
GATGCCTCTTCCTGCTGAGATCCCTCCAGTTTTTCCCAGTGGGAAGTTGGGACCAAGCACAAACTCCATG
TCTGGTGGGGTACAGACTCCAAGGGAAGACTGGGCTCCAAAGCCACATGCCTTTGTTGGCAGCGTCAAGA
ATGAGAAGACTTTTGTGGGGGGTCCTCTTAAGGCAAATGCCGAGAACAGGAAAGCTACTGGGCATAGTCC
CCTGGAACTGGTGGGTCACTTGGAAGGGATGCCCTTTGTCATGGACTTGCCCTTCTGGAAATTACCCCGA
GAGCCAGGGAAGGGGCTCAGTGAGCCTCTGGAGCCTTCTTCTCTCCCCTCCCAACTCAGCATCAAGCAGG
CATTTTATGGGAAGCTTTCTAAACTCCAACTGAGTTCCACCAGCTTTAATTATTCCTCTAGCTCTCCCAC
CTTTCCCAAAGGCCTTGCTGGAAGTGTGGTGCAGCTGAGCCACAAAGCAAACTTTGGTGCGAGCCACAGT
GCATCACTTTCCTTGCAAATGTTCACTGACAGCAGCACGGTGGAAAGCATCTCGCTCCAGTGTGCGTGCA
GCCTGAAAGCCATGATCATGTGCCAAGGCTGCGGTGCGTTCTGTCACGATGACTGTATTGGACCCTCAAA
GCTCTGTGTATTGTGCCTTGTGGTGAGATAATAAATTATGGCCATGGGAAACATTGTATATTTAGTGTGT
GTATTTTGATAATGATTGATCTTAAATCTGTATACAGAATATCATTGATATAATACTCTTTAGGCAGGAG
CACTCTTGCCTTCCCCCAAAATTTACACTGCTAAAGCCCTCTGTCACTTGGCGACCCTTCTGGTCTTGCT
GGAGGGGTTTCCTGGGTATAACCCATTGGGCTGCCCAAGGCCAGCCAGCCTGAGCTCTCCTGCAAGACAG
AGCCTGATGTGGCACGGAGTGGGGTTGCGGGGGGTGGGGGGACTGCCTGACTCCCAGAGGGACTTGAAAC
TGAAGCAAGAAGGTTGCATTCTCCACCAAGGGAGTTAACCTACCTGAACTAAGTAGAAATGCCAGTCTTC
CACTACCCCCTCCCTGCCATCTTTTCTTCTGCTACTTTGGGGAGTTGATGGCCAGGAAAGAAGCCAGCAC
AGGGTTAAAGTAACTCCTGGCATTGCCCACCAGGGGGCTGGTGCACCTGCTGACCTCAGGGTCACAGTTG
AGTCATTTGCCAGTTGACGGAGCAAGTTTGACCTTGGTTCTGTTGCTGAAGCAAATTTGGAACTTTTCTG
TCTCAGTGTGATCCACTAACCCACAGGATCATTTGGAACCTTGAATAGCTCTGCTTGGACAATGGGGTTG
GGGAATAGGGTTGTCTTTCCTATGAAAATGCCATCTGTAGACCTTGTGAGTCAGCCGTCCAGATGTTTGC
AGGTGAATTCCTCTGCTTGACATCCTCCCTGTCACTTTGGACCCTATGGGAGTGGGCATCTCCACGCACC
TGTGTATGTGAAAGTCATTTTACATTTCAAAGCAGTGTGTGTTTCTTATTTTTATATTTTTAACTCTTTA
TTCTTGGATGTATAAAGTGAACTTTTTGGCTTCTGTAAGTATGCTCTATGCACCTCTAATGTTTTATCAT
GTATTTATATGTTGTACACAGTACTGGCTGATTCTGTAAATGGATGTATTGTACAGAGAACATGAACGTC
TCTTCCTAATTTTACATCTTCAGCATCATTGCATTAAAGTGGTGTAATCTCCTTCTCTACATCTGTTGTC
AGAGCCACTGAGTGCTGTGCTGCTCGACGTGAGGGTGAAATGATTGACTTGTGACCTGCCAGGTTGCCCG
ATGCCCTGTTGGGTCACCGGCTGGACCTGCTGCAGCCTGCAGAGCCACAGTCAGCCTGCCCACATGCCAC
CGAGCAAACGCATCTTGCTTTTCACATCTCTCCTCCTACAGCCTTAATGGCTGCTTGCTGCCATATGTGA
CAAATCACCACCACCAGTGTTAAGTGCTTCTGGATTCATGGGTGAGTTCCCTGGGCAGCCCCCAGGAAGG
CCTTCCAGATCTGGCTCCAGGGTCACCACCTGTCACAGCAATACCTGGGACCATGCTCTCCTGGGACTGT
GAGGCTCCTTTTGACGTACTTTTGACATCAGGCAGGTTTGGGAAGAAACAAAGCCATGCCTGCTCCTGCC
TCTCTCCCAACATGTTTCCAGCAAGTAGATGCCCCTGTGTGTGTTTTCCCTTGCCTTGTTTCCTGCCTTA
TATCTTGTATTTCGACTTATTACAGAGTTGAGGGTTCTTGCTTAATTTAGATCAAGTATAAAATTTGTAT
GACTTCAAGTCTCATTTTATCTGAAAGGTTTTTTTCTCATTTAATCTGATGTGGCATTTTCGTCATCTGA
AGCATGAGTGACAAGTTGGGAATGATGTGGTGATTTAGAATGCAGTATTGGCCAAGTCCAAGTTGTCAAC
TTAAGCGTCTGTTTACCAAAGACCGGGAACAGGGGCCCAAACATGTCCAGTCCTCTTCTTCCCTCTGCTG
GAACCTTTGGGGACACTCAAGGGTACAGTTTGACACTGATCTGGTCCATGAGGCTGCCCAGAGAAAGCAC
TGCTTCTGTATGTCTCTTGTGGTATTGGAACAATAAACCCGTACAACCTGCA
SEQ ID NO: 25 (NM_001164603.1, AXSL1 isoform 2 mRNA):
CACACCCACGGCAGACACGCACGCACCCGGGCGCCGAAGGGAAAGCCGCGTCTCGCCCTCCCGCCCCGCC
GTCGGTCCTGTCTCAGTCCCTCAGCAGAGCGGGAAAGCGGAGGCCGGAGCCGTGACCTCTGACCCCGTGG
TTATGCGGAGCCGCCGCATTCCTTAGCGATCGCGGGGCAGCCGCCGCTGCCGCCGTGGGCGACTGACGCA
GCGCGGGCGCGTGGAGCCGCCGCCGCCCCTCCCCCACCGCCGCTCTCGCGCCAGCCGGTCCCCGCGTGCC
CGCCCCTTCTCCCCGGCCGCACCCGAGACCTCGCGCGCCGCCGCTGCCACGCGCCCCCCCCACCGCCGCC
GCCGCCCCAGCCCCGCGCCACCGCCCCAGCCCGCCCAGCCCGGAGGTCCCGCGTGGAGCTGCCGCCGCCG
CCGGGGAGAAGGATGAAGGACAAACAGAAGAAGAAGAAGGAGCGCACGTGGGCCGAGGCCGCGCGCCTGG
TATTAGAAAACTACTCGGATGCTCCAATGACACCAAAACAGATTCTGCAGGTCATAGAGGCAGAAGGACT
AAAGGAAATGAGAAGTGGGACTTCCCCTCTCGCATGCCTCAATGCTATGCTACATTCCAATTCAAGAGGA
GGAGAGGGGTTGTTTTATAAACTGCCTGGCCGAATCAGCCTTTTCACGCTCAAGGTGTGAGCCACTGCAC
CAGGCCCCTTCATCTTAATTTTAATATATCTTTGAATAAACACCATTGTATGAACCTGCTGTAAGCTTGG
GAGTGGTCTGTTAGTCTACAGCTTGTGTCTGAGATGTGCTAATTGAATATTTGCTCAGTACCTCATCTTA
ACTGCCTTTGGCTTTATGTTGCTTATCCTTCATAGTATCTTGTTCATTGGCCTTTTACATCCATAGGCAT
CACTTCTCTGATATTCGTTGTGCTCTTTTAATGGATTAATGGTTTGCTTGGTTGGTTCCTCTAGTTAGAC
TGTAAACTCCTTGAGAGCAGAGTCTGTATTTTATTAATTACCCACAGTACTAGGTACATAGTTGCCTTCA
ATAAATATATATTTAATGAAAAAAAAAAAAAAAA
SEQ ID NO: 26 (NM_001363734.1 , AXSL1 isoform 3 mRNA):
AAATTCCGGCGGAGGGGCGAGCTGGCAGGCCGGCTCCTCCCACTCTGGGCAGCGGGGTCCCGCGTCCCCT
CCCCCACTATTTGGCAGCGTCTGGGGGTCTGGGGCAGCTTCGTTCATTCACCCGGGGGAGTTGGGTTTCC
GGGAAGGGTCGGAAGCTCCTCCCTCGCTTCCTGGTGGGTAATGGGGTGGTGCCTTTGACTCCGGGGGTGG
AAAAGCGACCCCACATTCAAGGACGCCAATGGCATGTTGAGCTTTCCCAATCTAAACCAGGTATTAGAAA
ACTACTCGGATGCTCCAATGACACCAAAACAGATTCTGCAGGTCATAGAGGCAGAAGGACTAAAGGAAAT
GAGAAGTGGGACTTCCCCTCTCGCATGCCTCAATGCTATGCTACATTCCAATTCAAGAGGAGGAGAGGGG
TTGTTTTATAAACTGCCTGGCCGAATCAGCCTTTTCACGCTCAAGAAGGATGCCCTGCAGTGGTCTCGCC
ATCCAGCTACAGTGGAGGGAGAGGAGCCAGAGGACACGGCTGATGTGGAGAGCTGTGGGTCTAATGAAGC
CAGCACTGTGAGTGGTGAAAACGATGTATCTCTTGATGAAACATCTTCGAACGCATCCTGTTCTACAGAA
TCTCAGAGTCGACCTCTTTCCAATCCCAGGGACAGCTACAGAGCTTCCTCACAGGCGAACAAACAAAAGA
AAAAGACTGGGGTGATGCTGCCTCGAGTTGTCCTGACTCCTCTGAAGGTAAACGGGGCCCACGTGGAATC
TGCATCAGGTCAAATGAAGCGCAACAGAGGGGAAGAAATAGATTTTGAGACACCTGGGTCCATTCTTGTC
AACACCAACCTCCGTGCCCTGATCAACTCTCGGACCTTCCATGCCTTACCATCACACTTCCAGCAGCAGC
TCCTCTTCCTCCTGCCTGAAGTAGACAGACAGGTGGGGACGGATGGCCTGTTGCGTCTCAGCAGCAGTGC
ACTAAATAACGAGTTTTTTACCCATGCGGCTCAGAGCTGGCGGGAGCGCCTGGCTGATGGTGAATTTACT
CATGAGATGCAAGTCAGGATACGACAGGAAATGGAGAAGGAAAAGAAGGTGGAACAATGGAAAGAAAAGT
TCTTTGAAGACTACTATGGACAGAAGCTGGGTTTGACCAAAGAAGAGTCATTGCAGCAGAACGTGGGCCA
GGAGGAGGCTGAAATCAAAAGTGGCTTGTGTGTCCCAGGAGAATCAGTGCGTATACAGCGTGGTCCAGCC
ACCCGACAGCGAGATGGGCATTTTAAGAAACGCTCTCGGCCAGATCTCCGAACCAGAGCCAGAAGGAATC
TGTACAAAAAACAGGAGTCAGAACAAGCAGGGGTTGCTAAGGATGCAAAATCTGTGGCCTCAGATGTTCC
CCTCTACAAGGATGGGGAGGCTAAGACTGACCCAGCAGGGCTGAGCAGTCCCCATCTGCCAGGCACATCC
TCTGCAGCACCCGACCTGGAGGGTCCCGAATTCCCAGTTGAGTCTGTGGCTTCTCGGATCCAGGCTGAGC
CAGACAACTTGGCACGTGCCTCTGCATCTCCAGACAGAATTCCTAGCCTGCCTCAGGAAACTGTGGATCA
GGAACCCAAGGATCAGAAGAGGAAATCCTTTGAGCAGGCGGCCTCTGCATCCTTTCCCGAAAAGAAGCCC
CGGCTTGAAGATCGTCAGTCCTTTCGTAACACAATTGAAAGTGTTCACACCGAAAAGCCACAGCCCACTA
AAGAGGAGCCCAAAGTCCCGCCCATCCGGATTCAACTTTCACGTATCAAACCACCCTGGGTGGTTAAAGG
TCAGCCCACTTACCAGATATGCCCCCGGATCATCCCCACCACGGAGTCCTCCTGCCGGGGTTGGACTGGC
GCCAGGACCCTCGCAGACATTAAAGCCCGTGCTCTGCAGGTCCGAGGGGCGAGAGGTCACCACTGCCATA
GAGAGGCGGCCACCACTGCCATCGGAGGGGGGGGTGGCCCGGGTGGAGGTGGCGGCGGGGCCACCGATGA
GGGAGGTGGCAGAGGCAGCAGCAGTGGTGATGGTGGTGAGGCCTGTGGCCACCCTGAGCCCAGGGGAGGC
CCGAGCACCCCTGGAAAGTGTACGTCAGATCTACAGCGAACACAACTACTGCCGCCTTATCCTCTAAATG
GGGAGCATACCCAGGCCGGAACTGCCATGTCCAGAGCTAGGAGAGAGGACCTGCCTTCTCTGAGAAAGGA
GGAAAGCTGCCTACTACAGAGGGCTACAGTTGGACTCACAGATGGGCTAGGAGATGCCTCCCAACTCCCC
GTTGCTCCCACTGGGGACCAGCCATGCCAGGCCTTGCCCCTACTGTCCTCCCAAACCTCAGTAGCTGAGA
GATTAGTGGAGCAGCCTCAGTTGCATCCGGATGTTAGAACTGAATGTGAGTCTGGCACCACTTCCTGGGA
AAGTGATGATGAGGAGCAAGGACCCACCGTTCCTGCAGACAATGGTCCCATTCCGTCTCTAGTGGGAGAT
GATACATTAGAGAAAGGAACTGGCCAAGCTCTTGACAGTCATCCCACTATGAAGGATCCTGTAAATGTGA
CCCCCAGTTCCACACCTGAATCCTCACCGACTGATTGCCTGCAGAACAGAGCATTTGATGACGAATTAGG
GCTTGGTGGCTCATGCCCTCCTATGAGGGAAAGTGATACTAGACAAGAAAACTTGAAAACCAAGGCTCTC
GTTTCTAACAGTTCTTTGCATTGGATACCCATCCCATCGAATGATGAGGTAGTGAAACAGCCCAAACCAG
AATCCAGAGAACACATACCATCTGTTGAGCCCCAGGTTGGAGAGGAGTGGGAGAAAGCTGCTCCCACCCC
TCCTGCATTGCCTGGGGATTTGACAGCTGAGGAGGGTCTAGATCCTCTTGACAGCCTTACTTCACTCTGG
ACTGTGCCATCTCGAGGAGGCAGTGACAGCAATGGCAGTTACTGTCAACAGGTGGACATTGAAAAGCTGA
AAATCAACGGAGACTCTGAAGCACTGAGTCCTCACGGTGAGTCCACGGATACAGCCTCTGACTTTGAAGG
TCACCTCACGGAGGACAGCAGTGAGGCTGACACTAGAGAAGCTGCAGTGACAAAGGGATCTTCGGTGGAC
AAGGATGAGAAACCCAATTGGAACCAATCTGCCCCACTGTCCAAGGTGAATGGTGACATGCGTCTGGTTA
CAAGGACAGATGGGATGGTTGCTCCTCAGAGCTGGGTGTCTCGAGTATGTGCGGTCCGCCAAAAGATCCC
AGATTCCCTACTGCTGGCCAGTACTGAGTACCAGCCAAGAGCCGTGTGCCTGTCCATGCCTGGGTCCTCA
GTGGAGGCCACTAACCCACTTGTGATGCAGTTGCTGCAGGGTAGCTTGCCCCTAGAGAAGGTTCTTCCAC
CAGCCCACGATGACAGCATGTCAGAATCCCCACAAGTACCACTTACAAAAGACCAGAGCCATGGCTCGCT
ACGCATGGGATCTTTACATGGTCTTGGAAAAAACAGTGGCATGGTTGATGGAAGCAGCCCCAGTTCTTTA
AGGGCTTTGAAGGAGCCTCTTCTGCCAGATAGCTGTGAAACAGGCACTGGTCTTGCCAGGATTGAGGCCA
CCCAGGCTCCTGGAGCACCCCAAAAGAATTGCAAGGCAGTCCCAAGTTTTGACTCCCTCCATCCAGTGAC
AAATCCCATTACATCCTCTAGGAAACTGGAAGAAATGGATTCCAAAGAGCAGTTCTCTTCCTTTAGTTGT
GAAGATCAGAAGGAAGTCCGTGCTATGTCACAGGACAGTAATTCAAATGCTGCTCCAGGAAAGAGCCCAG
GAGATCTTACTACCTCGAGAACACCTCGTTTCTCATCTCCAAATGTGATCTCCTTTGGTCCAGAGCAGAC
AGGTCGGGCCCTGGGTGATCAGAGCAATGTTACAGGCCAAGGGAAGAAGCTTTTTGGCTCTGGGAATGTG
GCTGCAACCCTTCAGCGCCCCAGGCCTGCGGACCCGATGCCTCTTCCTGCTGAGATCCCTCCAGTTTTTC
CCAGTGGGAAGTTGGGACCAAGCACAAACTCCATGTCTGGTGGGGTACAGACTCCAAGGGAAGACTGGGC
TCCAAAGCCACATGCCTTTGTTGGCAGCGTCAAGAATGAGAAGACTTTTGTGGGGGGTCCTCTTAAGGCA
AATGCCGAGAACAGGAAAGCTACTGGGCATAGTCCCCTGGAACTGGTGGGTCACTTGGAAGGGATGCCCT
TTGTCATGGACTTGCCCTTCTGGAAATTACCCCGAGAGCCAGGGAAGGGGCTCAGTGAGCCTCTGGAGCC
TTCTTCTCTCCCCTCCCAACTCAGCATCAAGCAGGCATTTTATGGGAAGCTTTCTAAACTCCAACTGAGT
TCCACCAGCTTTAATTATTCCTCTAGCTCTCCCACCTTTCCCAAAGGCCTTGCTGGAAGTGTGGTGCAGC
TGAGCCACAAAGCAAACTTTGGTGCGAGCCACAGTGCATCACTTTCCTTGCAAATGTTCACTGACAGCAG
CACGGTGGAAAGCATCTCGCTCCAGTGTGCGTGCAGCCTGAAAGCCATGATCATGTGCCAAGGCTGCGGT
GCGTTCTGTCACGATGACTGTATTGGACCCTCAAAGCTCTGTGTATTGTGCCTTGTGGTGAGATAATAAA
TTATGGCCATGGGAAACATTGTATATTTAGTGTGTGTATTTTGATAATGATTGATCTTAAATCTGTATAC
AGAATATCATTGATATAATACTCTTTAGGCAGGAGCACTCTTGCCTTCCCCCAAAATTTACACTGCTAAA
GCCCTCTGTCACTTGGCGACCCTTCTGGTCTTGCTGGAGGGGTTTCCTGGGTATAACCCATTGGGCTGCC
CAAGGCCAGCCAGCCTGAGCTCTCCTGCAAGACAGAGCCTGATGTGGCACGGAGTGGGGTTGCGGGGGGT
GGGGGGACTGCCTGACTCCCAGAGGGACTTGAAACTGAAGCAAGAAGGTTGCATTCTCCACCAAGGGAGT
TAACCTACCTGAACTAAGTAGAAATGCCAGTCTTCCACTACCCCCTCCCTGCCATCTTTTCTTCTGCTAC
TTTGGGGAGTTGATGGCCAGGAAAGAAGCCAGCACAGGGTTAAAGTAACTCCTGGCATTGCCCACCAGGG
GGCTGGTGCACCTGCTGACCTCAGGGTCACAGTTGAGTCATTTGCCAGTTGACGGAGCAAGTTTGACCTT
GGTTCTGTTGCTGAAGCAAATTTGGAACTTTTCTGTCTCAGTGTGATCCACTAACCCACAGGATCATTTG
GAACCTTGAATAGCTCTGCTTGGACAATGGGGTTGGGGAATAGGGTTGTCTTTCCTATGAAAATGCCATC
TGTAGACCTTGTGAGTCAGCCGTCCAGATGTTTGCAGGTGAATTCCTCTGCTTGACATCCTCCCTGTCAC
TTTGGACCCTATGGGAGTGGGCATCTCCACGCACCTGTGTATGTGAAAGTCATTTTACATTTCAAAGCAG
TGTGTGTTTCTTATTTTTATATTTTTAACTCTTTATTCTTGGATGTATAAAGTGAACTTTTTGGCTTCTG
TAAGTATGCTCTATGCACCTCTAATGTTTTATCATGTATTTATATGTTGTACACAGTACTGGCTGATTCT
GTAAATGGATGTATTGTACAGAGAACATGAACGTCTCTTCCTAATTTTACATCTTCAGCATCATTGCATT
AAAGTGGTGTAATCTCCTTCTCTACATCTGTTGTCAGAGCCACTGAGTGCTGTGCTGCTCGACGTGAGGG
TGAAATGATTGACTTGTGACCTGCCAGGTTGCCCGATGCCCTGTTGGGTCACCGGCTGGACCTGCTGCAG
CCTGCAGAGCCACAGTCAGCCTGCCCACATGCCACCGAGCAAACGCATCTTGCTTTTCACATCTCTCCTC
CTACAGCCTTAATGGCTGCTTGCTGCCATATGTGACAAATCACCACCACCAGTGTTAAGTGCTTCTGGAT
TCATGGGTGAGTTCCCTGGGCAGCCCCCAGGAAGGCCTTCCAGATCTGGCTCCAGGGTCACCACCTGTCA
CAGCAATACCTGGGACCATGCTCTCCTGGGACTGTGAGGCTCCTTTTGACGTACTTTTGACATCAGGCAG
GTTTGGGAAGAAACAAAGCCATGCCTGCTCCTGCCTCTCTCCCAACATGTTTCCAGCAAGTAGATGCCCC
TGTGTGTGTTTTCCCTTGCCTTGTTTCCTGCCTTATATCTTGTATTTCGACTTATTACAGAGTTGAGGGT
TCTTGCTTAATTTAGATCAAGTATAAAATTTGTATGACTTCAAGTCTCATTTTATCTGAAAGGTTTTTTT
CTCATTTAATCTGATGTGGCATTTTCGTCATCTGAAGCATGAGTGACAAGTTGGGAATGATGTGGTGATT
TAGAATGCAGTATTGGCCAAGTCCAAGTTGTCAACTTAAGCGTCTGTTTACCAAAGACCGGGAACAGGGG
CCCAAACATGTCCAGTCCTCTTCTTCCCTCTGCTGGAACCTTTGGGGACACTCAAGGGTACAGTTTGACA
CTGATCTGGTCCATGAGGCTGCCCAGAGAAAGCACTGCTTCTGTATGTCTCTTGTGGTATTGGAACAATA
AACCCGTACAACCTGCA
SEQ ID NO: 27 (NC_000002.12: c25342590-25227874 Homo sapiens chromosome 2,
GRCh38.p13 Primary Assembly, DNMT3A genomic sequence):
GCAGTGGGCTCTGGCGGAGGTCGGGAGAACTGCAGGGCGAAGGCCGCCGGGGGCTCCGCGGGCTGCGGGG
GGAGGCACTTGACACCGGCCCGGGGAGAGGAGGGGCCGCTGTCCCTGCGGCCAGTGCTGGATGCGGGGAC
CCAGCGCAGAAGCAGCGCCAGGTAGCGGGGCGCCCAGCGTGGGGAGGGCTGCGGCGAGGCCCGGAGCTCG
GCTACGACTCGAGGGGCTCTGAGTGGCCGGGAGCGCGGCCGAGGCGGGGCGCCCGGGGCCCGGCCCCCGG
TCTGGGCGGTGCGGGGTCCAGACCTCCGCCAAGCGGCGTGCGGGGACCCCGGCGGGGCCGCAGAGGCCGG
GAAGTCGCGCCGAGGGCGCTAGGACCGGCCGCGCGCCCAGGCCGCTCCCGCCGCGGCGCCCCGGGGCCCC
CCAGTCGGCGCTGCCTCCCGGGCCGGCTCCGCAGCGCCGGGGCCGGGGCCAGCGCGGCCGCGGGGGGCGA
GCGGCGGGCGGAGGCCTGGGCCCGCGGGCGGCCGGAGGGAGGGAAGGAGGAAGGCGGAGAGAAGGGGTGG
GAGGGGGGGACCCGGCGGAGGAGGCGGAGAGAGGGAGCGCGACAGCGAGCGGAGGGAGGGAGCGAGCGAG
CGAGGCAGGCAGGCGGGCCGGGAGGGAGGGAGGGCGCGCGGGCGGCGGCGGCGGCGAGAGCAGAGGACGA
GCCGGGACGCGGCGCCGCGGCACCAGGGCGCGCAGCCGGGCCGGCCCGACCCCACCGGCCATACGGTAAT
GAGCGCCGCTGCTGGCGGCCCGGAAGGCCGGCCCGGAGGCGGCGGGGAGCCGGGGAGCCGGGCGGGGGGC
GCCGCGGCTGGCAGTGCCCGGCTCGGGGCACGCTCCCGGACCCCTGCAACTCGGCGCCGGGCGCCCGTGG
GGCCGGGACTGCAGGCGGGGACGGCGGGCCGGGCTGGCCGGGGCGGCGCGGGGGATTTGAGGGTTTGTTT
ATGTCCCCGCGGGCTCGGCAGGCGCCCCCTCCCGTCCCGCCCGCTCCCGGCGCGGACCCCCCTCCCCGCC
CCCGCCCGGCTCCGGGCTCAAGTTCAAGTCTATACAATGCCGCGGGGGGCCCCTCCACCGCCGGCCTGCG
CCGCGGTGCCCGGCTCGCCGCTGCCGCAGGGGCCCCGGCCGGGCCGCCCCCGCCTTCCGGGCTCTGCCCC
AGCCCCGCGGCCCCGCCTGGAGCCCGCGGCCAGGGCGCCGGAGGGCGCGGACCCCGACCCAGAAGGATCG
GCCCCCGCGGCGGCCCGGGGGAGCGGGGGAGGGGGCGGGGGCCGGGCCCCGCGCAGGCTCCGCGGCGGCG
GCAGAAAATGGCAGCCTGGCACGACCCGGCCCCCCGCCCCTCCCCCCGGCGCCCGGCCCTCCTCGGCCCC
GCGGGGCGGGGGACCCGGGAGGCCCCCGGGGGCGGGCCGAGGGGTGATGGCGGGGCCGGGCCGGGGAGCG
CCGCGCCGCGATCCTCCTCCGGCCACCTGTGGCTGGGGGTGCGGGGCGCCGGCGGGGCCGGGAGGGGCGG
CGGGGCCGGGGAGAGCGCCCGCCTTGGGGGCACACCTTCCCCCGGAGTGCCGGCGGCACGGAGAGGGGTG
GTCGTGCCCGGCTCCTCCCTGTCCCCACCGCGGGCCGTGGGTGCCCGGGCGGCCGCGGTGGCGGCGCCGC
AATGTGGGGGCGGGGCGGGGGCGGGCGAGGATCCCGGGCCCGGGCCCGGGCCGCGGGGGCCGGAAAACGC
GGGGGTGGAAGTAGCGGGGCCGGGACCGGGAGCGGCGCAGGGGGGGTACGCCCGGGGTCGCCCCTCCAGC
ACCCCCGCGCACACTCCCACGCACGCGGACACACTCACGCACACGCACCCCGCCGGGCCGGTGCAGACGT
GTCCTTGAAAGCTTCGCATCTGCACAGTGAAGGACGAGGCCTCGCCGCGGCTGCGGAGCGGGGTCTTCGG
CCCCACGTCCCCGCACCCCGGGAGACCTCGAGCTTCGGAGCCGCAGGCGGCGGCTGTCCGTGTCCCGCCC
TCACCCCAGTTCCCCTCCCCCAGGCCGCCCTGCCTTCCCTCCCCTTCGGCGCGGACCGGGCTGGCGCGGG
TCCAGGGCCCGCCCCTGGCCTCCCCCAGCCCCTCATCGCTGGGGCCCGCGGCCCTGGGGCTGCCAAACTG
CTTTTCGCTTTCCTGAAGCCTCCCTTCTTGGGGGCACCCTCTCCCACCCCAGCGCCCCTCTGCCATTTCT
CCCCCTCTCCTCATCCCCTCCGTCCTGGCACCCTCTCTCCGCTGCACGGCCAGTTGAGGAGGTGTTGGCG
CCCTGGGCGGGGAGCAGTGCCCTCTGGTTCCCAGGGGCTGCCTCCCCCGGCTCCTGAGGGGAGCCCCCTG
CGGCTGCACCTTGACCCAACATGGCGTCTGGCACCCAGCAGGGCAGCTCTGGCCTGCGGCAGGCGGGAGG
GTGGGCAGGGCCGAGCCCCAGGGCCGCGCAGCTACCCCCCCTCCAAACCAGCGGGGACAGAAGCCTTGGG
GAGAAGCGCTGTCCGCCCTGTGACCCAACTTGCCCCTCACTGCCGTGCCCAGAGACTGCCATCAGCATCC
CTGAGGGCCTGCTCAGTCTTCATGTTTCACCCTAGGCTGCCAGCGGCTGAACCCGCCTCCTGGGCGGGCG
GCTACGAAGGGATCCTGGGATCAGGTGCTGCTCCAGTGTCGCCTCCTCCCTCGCGGGCAGATGTGACAGC
TGCAGCCAGGGGAGCTATTCGCCTTGCTATTGCCTGGGTGTTAGGACTGGCCTGTGGGATTTGGGGGGCC
AGGCATCCCGGGGACCCTCTTTGGTTGGCTAGCAGGGGCCATCTCCAGGTAGTTTTCTGAGCTGCCTGCC
TTTGGAGAGTTGGGTTGTAGGGAGTTCCTGCTGGGTTTCTGGGGCAAAATGGTCCCCCTCTGCCTCCAGG
TTCTACCGAGGCCCTGCTTGGGCTGTGACAGGTGGCTCTGTCAAGGTGGGAGTCTGTGTGTGCACATGTG
CTGGCTCCAGGCGAGGGACCAGGCTGGCCTGTGCAGCAGCTCTGGCACCTGCCTGCCTCCCTGGTGGGTC
CCCCTCCTCTGTGCAGCTCCTCCCCAGATAAGCAGCTGCGGATTCAGGCCGACCAAACTTCCCTCGTTAC
CCCACCCAGGCACCAGTGCCTTCCATAGGAGACTGACCCTAAGAAAGGTGAATTCCTTCTAGACTCAAGG
AGTGGACTGGTCCACAGGGCTGCCCTCTGAGTCCTTCCTGGAGAAGCCTAGGAAAGGACGCCAGCAGCCA
TCTGTCAGGGTGTCTGCGGACAGGATTCCTCTGTGGGGTGGGGGTTGGACTAGAGCCGGAAGGTCCATTC
CAGCTTTGACATTCTATGGCTCTGATTCTAGTGGTGGAATTTCAGGCCTAGCAATGGGGACAGGGTGGTC
TGTTTTTCGGCACATGGCAGTAATAGGGGAAGGCTTCCAAGGAGAAGGAGATATGAGAGAGATCAGGGCG
CCCAGCGAGTTGGGCCTTGGGAAGGAGAGGCAGTGTGAGGAGGGTCCCTGCAAGAGACGGGTTGGTCACG
TCCGACTCTGCAGAGGGTGTAAAAAGTACAGCCTCCTTTAAGCCTCGGCACGTGTGTATCCTGTAGGTTT
GTGTATAAATCCCTGTGTGTGGATAATTTCTGAGGTTTCCTTTTCAAGTCCAATTCACAGTAAGCTCCTC
AGTCCCTTATGGATGTTTTACTATCGCTTTCACACCCACCTCTGCCAGTGGTTCTCAAAAAGCTAGGAAG
ACAGGACAAACGGTAAAATCCTTGTCCAGGGTCACCGGCAGGAAGAAGTGGAGCCAGGCCTGCACTCGGG
GGAAGATGCCCCATGCTATACCTCTTACCTGGGCTGCTGCATGTGTCTGTCCCCGCTCTCAGTTTGTGAT
CTCCAGGAAACAGGGGCCTTGCCTCCCTGGGTCCCCATTGGACAAGCGGTGAGGCCTCCGAGGGTGTCTG
CTGAGCGAGGGGAGCGCACTGCCCTCCAGTCCTCTCCCCGAGGCAGCGAGGCAGGTTAGTGCTGGGCTCC
CTTCCCCCTGCTGCTGTGCGCCGCTGCCCAGCGGCCACTGCCTACTTACAGGCCAGCTCCTCAGGGTCAC
ACCTCCCTGAAAGACCCAAGTTCTTTCCCAGCAAGAGCCAGACTCCCCCAAGGCTTCTCTTTTTGGGATC
CAGGTAGAAAGAGGACTCTAATGGGTGAGTTAAAACAGACCACCCTCTCCCCATCTTTGGGCCCGAAATG
TCAGCATTAGAACCCAAACCTTAGAATTGCAGTCTGAAAGAGGAGTTTAAGCTCTGAGAGCTCTTAGTGC
TGGGTACGGGACTGTTTGTGGGAGGTGGTGCCCAGTTCTTCCCAGGGTTGTCCATGCTACCCAGAGCCTG
AGGCCTGGAGGGCCATTCACGTGCCAGTCACATGCCTCTCCCAGGGTACTTCCTGCTGCCTCCAACTTGC
TGGCCTGGGGTCCTGGACTTGGTGCTCGTATCCTCCCCACCCCGTGCCCCACTGGGAGTTGAGTGTGTCT
CTGGCCTCCAGCAGGTAGGGCCAGAGCATCTACCCCAATGCCATTCCCCATGCTGCGGCCCTCGGGGAAT
AGCTTCTACTTTTGTTTGAATATTAGTATGTGAGCAGAGAGCCCGTCAGAAGCCCACCTGGCCTGAGGCT
GGGTTTCTGCAGGGACTTTGCTGTTCCTGAGCAGGGCTTTAATGGTCTACTAAAGGTGCCAGATCCCTTT
CCACTTTAATTTTGAGGGCTCATTTTCAATCATTTCTCTCTGACGATTTTTCCCTAAGAACATGACCAAA
TCTGAGATCCATGAAGTGTCAGCCTGAGATTTATGTCTGTATCCGAGCCTGCCTCCACTTGGCTAAGTGT
GTGGGCAAGACGAGGTTCAGTGCACATGTGTCTGTGCATGATGTGTGTGTACGTGGTGTGCCGCCACCTG
TGGATGGGTAGGACTTGCTTCCACATGAGCCAGTGTCAAGGCAGCTTGAGGCCCTCGAGTCACAGGGAGC
TGCACTCTGACACCTCCCAGCGGCAGGAACTTGGGCAACAACTCGATTCCCTGGGCCTCAGTTTCCTCAC
CTGTAACATGGGAGTGTGCGTGGATTCTGCTTACAGGTGGTTGTGAGGATGGAGTGAGATCATGTGCGTT
ACAGCCTTAGAGGAGCGGGAGCCCTCCTGTTAGCTGTGTCTGTTTTCATCATTTGGGTGGCTCACGTGGA
CTAGCACTGCAGGAAGTGCTCTAAGAAGCCTCGTCCACCTTCCTGTGTCTTTTGCAGAATCTTGGTCAGA
TGAGGAAAGTGCCGATTCTGATTAAAAATCCTGGGAGGTGGGAGGCTTCTCAGCGACACTTAGCAATCTG
TTGAGTTGAACAAATCTCGTGTCAACAGGTTGTTTTGCTTCCACCTGAAATCCCTCCCGCTGTAGTTTCA
GCTCGTTATTCTCATCCTGTCTTGAGTGCAGCAGCACTTTCCCACCGTATTCGCTTCCATTTTCCAGAGA
TGTGCTGAGCATTTCCTGTAAGCCATGCCCTGTGCTGGCTTTGAGGGGACACGGTGTGTTTAGGATGCGC
CCAGCTAACGAAGAACTGCAGAGTGGGACTGGGGTCACTGGAGAGGGAGAAATAAAGTGCTCAAAGGAGA
GTGAGGTCACAACCAACTAAGGTCATCAGGGAAGGCTTCTTGAAGGAGGTGGCATCTCAGAAGCGCCCCA
GAGCACAGAAGTGGGGCTTCAGGGGCAGGACATCATTGTGGGTGGCTGTAGTGCCCGGGAGGTGGACTGG
GGCCTGGGTTGTGCTGGAGGCCAGGCTGAGGCCCTGCCTTGGTTTGGGGAGGAGATCCCTGCACTCCGGA
ACTCCTCTGTGGCCCACGGAGGATCGCTCTGAACTGCCTCAGCGTGGCGGCCAGTGGGGGTAGGGGTGGA
GAGAGGACAGCAGGGAGGGAGGGGAAGGGGCCAAATGAGCTCATTTGGTGTCGGATCCTGATTCTTTCTA
TTCCATAGACCAGAGCTTAAGAGCTCAGACTTGGAGAGCAACAGAACAGGGTTTAAATTACACTGACCCC
AGGGGGTTGTGACGATTAAGGGGGAGGCCAGGGTCAGGTCTGGTCTGCGAGCGAGTGATAGCAGTCATGG
TTGAGGATGTGGCAGGTGGAGAAGGGTCCCTTTTCTGGCCTCCCAGAGGTCTCCATGTCCCCCCTTGCCC
ACTCCATCAGGCTGAGGCGCCCCAGCATCCAGAACATTCCTGCAAGGGGCTGCCAGTGCCAGGGAAATGG
GGGTGAAGACGTCAGGTTCCTGTCTCTGCAGCCTGACCCTGTCCCCCAACACACACGCTGGGCAGAACCG
AGCCACCAACCCAGAGGGGAGGTGCCCTGCAGACGGCCCAGGCCCCCAGGCATGAGTGCAGGTGCCTGGC
AGCCCCCTGCAAGAACAGCTGGTGGGCAGGGCCTTCTGGGCCAAATCTCCCTTGCTGATCACATCACAGG
CCACCAACCTGTCTGGAGGACAGCAAGGGTGCTTGGTGGGCAGTGGAGGTGGGGGTAAGGGAGGTGCAGA
CGCCTGGGGCTTTCCTAGCTAAATCCCAGTATGGAGTGGTTTGTTTTTTTTTTTCAGCAGCAGCTCTATG
TTGCCTACTCCTGTCCTACTGTGGTCCTCTCTGACCCTTAAAGGCTGCCCAGCCTTATTTAGATATATGA
CCAGGTTCCTATTTTCTCTTTCCTGTGGTCACGGCCCTGCTCACCACCATCCCTATTGATCTTTCCTCTC
TGCTTCTGTTTCAAATGTCAAGGTCATTTTTAATTCTGAGCCAGTTATTAGCTGCCCCGTCTGAGTGCAC
AGAGGGGACGGAGGACCATCTCTTATAGCCAGTAACCGGAGAGCTGGAACTTGGGTCTCCTGACTCCTAG
TCTAATGCTCTTCTTACTATAATGAAAGGGGAGGGGGGCTCAGTGCAGATGTTCGAGATCTGATAGATCA
GCTGGGTCATGGGTTCCAGTGATCCTTCCTTGAAAAGGTAAAAAGATGCTTAGAGATTATGGGGTGTGGG
TTAGATTTGCATCTCACTAAGCTGTTGAGTGTGGGCTGTGTCCTGGGGAATACTTGGGAACGGCCATGGT
CTGGGGGCAGGAGGCCTTCGGAGTGTCCCTCCCCCGCCACTCCCTGGAGGACCCCGGGAGACACAGGCGA
GTTCGCCCCTGCCCCTGCCAAGCCAGTGGGATTCCCCAGCACCCCAGTGAGCAGAACCTGTTTGGAAAGA
GGCTTCTGGAAGGCCTGCACGATTACTTGTATAGGTTCCCGAAGTGATAAATAAATGCATGGGTAAAGGG
GAATCCACTTAGATTCCCTAAAAGCCTTTGAAACAAGGTGGTAGGCGAGTCTGTTCTGATGTGTTAGATC
CCCATGCAGTAGTGTTGTGGTTAAAAAAAAAAAGTTCATAGTGTAATCTCAGTTTTGTGAAATTCTTTAA
AATCGATACAAATATACACATGTTTAGGTTTGCACATGCATGGAAAAGTTGTTCTGGAAAGATACACACA
AACCTGTCAACAAGTTTTTGCTTTTTAACTTTGTACACTTTCGTTTTCTTTGAAAAAATGTAAATGAACT
TGAGTTACTTTCTTAATGACAACAACAAAAATAAAACTACACTTTTTAAAAGCCTGTGGATGAGAAAGTA
GAAAAAAGAATTAAGTCACCTAAGGCTGAGGGATGGACTCTTTTTTATAATGGATAAGAAACTGGTTTAG
AGACTGACAGCAAGGGGTAAGCTTACTCAGGTACGTGTCTTTTCCGGAAAGGTTGCAGGGAGAAATATCG
CAGATATTAAGGCCTCTGAGCAAAGGCCTAGGCTGGTAGGGATTAGGTGAGAGCAGAAAATAGGCCGCTG
ACTGCATGCACCCTGGCCCACGCCAGGGACAGGGGACAAAGTTAGCTTTATAGGATGGCGACTTGAAACT
CATTTCTGACCCAAAGTGGAGAACAAATCACTGAGGATCTTCCCCCAAAAGCATCAGTCTGATGGGCTGT
TGTGGCCCAAATGGTTCCTGGGGAACTGGAGGTATAACTGGGGCCCGGCCCGGGTATGGAGCTCAGGTGA
CCCAGCCCTGGCATAGTGAGAGCCTCTTGGGAGGCCACACCCAAGGGAGACAAAGTGGGGCTTCAGGAGG
CCCCACGAAGGGGTAGAAGGGGTGTCTAGGAACTTGCTTGGCACGTGGGTTCAACTCTGATCTAGGGGTC
CCTGAGCAGGTGGTGATCACCCAGATGATGGGGTGGAGAGGTGAGGTGGGGGCAAATACAAGTTCCTCTG
TCTGCAGTCGTCTTGCTACCCCAGGAAGCTCAGGGCTGGTTGCTTAGCAACAGCACTTACCACCCTCTTC
CACCTTACCCTCCCGCACCCCACCCATGGGTGCCTCTTGGGGCTTCCCTTCCCCCGAGGCTGCCCCTGTT
CAATAGGACATGGGAAGGGTCTGTGCACTTTTTTGACCCTGCCCCTGGAATAAGGCAGGAACAGACATAA
AAGGACCTATGACTTGGAGGCAAGCACAGAACTGGCCCCTCTACAAACCAGCTGTTTAATCCAAGGCTGG
TAGGGGACAGATGTGCAGGGCAAAAAAGGCGTTCGGGAGTCAGGCCGACCTGGAGGACTGTCTCAGCCCC
ATTTGTGCTCAGTTGCTGCTGGCCCCAAACCACCTGGAGCTGCAGGTTTTTCATCTGTAAATCGGGGATA
AGAATAGCAGTCTCATCTTACAGGGGTTGCCTGAATGACGTGCAATATTTTGTGAAACAGCTAGCACTAT
GCCTGGCACAGAGAAGGTGCTCAGGAGGCATGAGCTGCTTTTCTTTACGTGGGGGTTGTTCCAGGGCTGC
CTGGTTAGGGTGACCCCCACAAAGCCCTGCTGCTCTGTAAGGCCCTGCTGCTCTGTAAGACCCTGCAAGT
GGCCAGGTGCAGTGGCTCACGCCTGTAATCCCAGCGCTTTGGGAGGCCGAGGCGGGTGGATCATCTGAGG
TGAGGAGTTCGAGACCAGCCTGGCCAACATGGTGAAACCACCTCTCTACTAAAAATACAAAAATTTAGCT
GGGTGTGATGGTGCACGCCTGTAATCCCAGCTACTCCGGAGGCTGAGGCAGGAGAATCACTTGAACCCGG
GAGGTGGAGGTTGCAGTGAGCCGAGATCGCGCCACTGCACTCCAGCCTGGGCGACAGAGTGAGACTTCGT
CTCAAAAGAAAAAAAAAAAAAAGACCTGCAAGTGTGGCCTGTCCTCCCCATGGCCCCTCACGGTGAGGGG
AAGAGATAATCAAGTTGAGAATGACCCCCAAGCCCATGGTGGACTCTGGGCTGGGAGTCAGGGGCCACAG
GGGAGTGGCAGGGATAGAGAAAGATGTCTCTGTCCTCCTTAGCTTCCGTTATCTGACCCAGGTCCCTTTT
TCCAGCTGCCAATAAGTTGTCCCTTTTTCTCTTAGCCTTTCTGCACCTCCACTTCTTCCTGTGAAGCCAG
GATTTGCCTGTACCTTTTTTGATCTCTTCAGAGTCCCCTGTCCATGTGGGGACCCAGTAGCCTGCTCAAG
GCCTGGGCCCTCTTTCCCTCCCTGCCCACTGGTCTCTTTGGAGCTAAGCGGAGAGTGGCCTTTGCTGGGT
TCTGGCTCTGCCCCACGGCCTGGAGGCAGGGGAGGGTCTAGACCACAGCCCGGCCTGGCAGCTGGCACGC
AGCAAGGGCTCCTAATGGTTTTATTACCAGCAACAATGTGGTGCTTGTTGCCATCCACGCACAGGCTGGT
GGTTCCCAGCCCAACTGGTGGTGGCCTGAGGGCCCTGCCTGGTACTGGAAGCTCAGCTGGGAAGCCCAGT
GGGGCTGGGCAGTCAGTCACCTCTGCGTTTTCTTCTGTCAACACCCTGCCAGCAGCCTGGAATCAGAGGG
AGCCAGGGCACGGCATGGCGAGATGCTGGCCCCCGAGTTGGCAGGCCTGGCAGCACTTGAGTTTTCTTTC
CAGAGGTCCCTGCCTCAGATCCACAAATGATGTTCATCCATTCATCTAAGAGACCTACTGTGTGCCAGAC
ACAGGCTGCATAGATGAAGGGGGTCACACTCATCTCAATGACCCTGCAGCTTAGGTGGGGTCTGGCAGGG
CGGAAGGACCCCTGGCACACCATGTCCAGGGATTACCAGGGCTCAGAGAAGGAAGTCGGATTCAGGGGGA
GAGAAGATCTGGAAGGGCTTTCTAAAGGAGGTGACGCTTGAGCTGAGTCTCGAAGCGGAGGGAGCAGTTA
GCTAGGTGGACGACGGCACTTTCTGGCCCCATGTGGCTGCAGTGTAGGGAACGATGGGATACGAAGCCAG
GGGCCTGGGCCGGGGGACATGGAAGGCGGGATGTTATGAACGGACGTTGGGCTTGGGATTTATATTTGAG
AAGGATCATTCTAGGGCATCGAGGACGAGAGCTCGGGCAATAGGGTTTAGGGGCAGGGAGCCCGGGTGGA
AGGCTGGCACAGAAGTCCGGGGCAGCAATGTGGGCCTGGACAGGGTGTGCGTGGTGTGACATTTAGGAAG
TGCAGTCAGTAGGAACAGGCGGGATAAAAGCAATGAGGGGAGTTGGGGTGGCTGTCTGGTTTCTAGCTCA
GCCAACTGTGTGGGTGGGGATGCCCTTCATGGAGGGGGCATGGGTTGGGGGGAGTCGGAGAGAGCAGGAG
GTGCCTCCCACATGGAGCTGGGTTGTAGCTGGGGCTTGAGGGCGGGTGGGATTTCCCCAGGGGGAGGCTG
AGCCAGGCCAGGCTTGCAGAAGGAGGCTGGAGCCTGAGTCATAAGGCCTGGAGGCGGGGTGCCTTGTGCG
ATGGATGAGAAAGGCAGGTGCATTTCAGTCTGTAACTGGTACGACCCCTGCAGATGTTTGAAGGAAGATT
TTGAGGAAGGTGCTCGCACCCTTGCATTGGGAATGCTGATGAGTTAGCGCCTCTACTTCACAGTGATTTC
CCCAAAAGATGAGCTGCAAGGCCTAGACCCTGGCCCTCCACCCAGGACCTGCGGCGAGCATCATCACCGT
CTGAGAATCTTTGGGTGGGCTGGAAGTTGATGTCAAGCTGTCCAGCCTTTCCGTCACTCTCCATTCAGAG
GCAGGTCTGCGGAGAGAGATAGGAGGGACAAGCTGGGGACAGCATTTTTGGAAAGCAAACAGAAAAGAAG
CAAAAACTGTTCCTCTAAAATAGAAAGGAGGCTTATGTGTGTTATGTGAAGGATAATGTGGAAGGCTATT
TCCAAAGAATTTCTCAAAAAATGACGTGAGCCATGTCAGCATCATTGAATTTAGATAGAGGCCAGGAGCA
GTGTGGCATCGTGCTGGGGCCACCAGGCTGGGGACTGGACAGATGATGGTTTCACCTCACCCCTGCCACC
TCCTTGCTGCGTGTCATTGGAAGATCACTCAACCTTTCTTAGGCTCAGTTTTACCATCTGTAAAATGGAC
ATAACTTCACAGGCTGGTGTTGTTTGTTTTGAGCATTGCTTCTTGGACTAAACCTGGATATTTTTTCCTT
CCCTCCCTCGCTTCTGCACTTCCTCTGGTTCTTTGCCTCCCAAGAAGCCTGTTTCCTCCTGTTCCCTCCA
AAACTCCAAGTTTTGAGTTCCCCCTTCCTGGGAAGGTCCATTCGTCATGTTCTATTGACTCTCTTGTTCC
CAATTATTCCCTGCAAACCTGGTTCACCCAGTGTATTTGAAGAGCATCTACTGAGCTAAATGGGAGTTAT
TGGGGGTCAGTTCCTGTTTTCTTTTTAATAAGGGGAGGGAGGGTCTGTGCAGAGCTGTCCGGGAGAGCAG
AGGGCATGGGGGACAGGGGGGATGTTGCAGGACCCAGTGGGACCACAGCTGGGACCTTGCCCTGTAGCTG
GCTCAGTGTGCCAGGGGATAGGGGCACAGAGCCCTGGTGGGTCTGGGAGATGAGCTTGGGAAGGACTTGG
CCTGGACATGGGCTAGGCTGCCCTGCTTTCCGAGCCAGACCTCAGGAGATGGCAGAGAGGGCCGCTTCCT
CTGTGAGGGCCGTACATGATGGCCCTTGGGCTGTATGGTGGCCTTGTGACATGCAGCCACACCCCTGGGG
AGACCTTTGAAGCATCTCTGCATTCTGGAGGATCTGCTGGGAAGATGGCTTCGCAGCGGGGCTCTTGTTG
ATGCTGCCAAGAAGGCCGGGTGGGAGTCGGGCTGTCTCTGGCTGGGGGTGGGGTGGGGGCTGCGGTCCAT
CCGAGTGTGGAAATCCACGTTTCTCTGGTTTGTGCAGACCTGTGCAAGCAGCCATGCTGCAGGACTGGCC
CCCTGGAAGTGGGGCATACATGAACTCAGGGTGTGGCGTTGTAATCAAATGAAATGTACAGCATAATTAA
TCTGGCATACCCTTAATGATATGCATAATTGTGCTGCATGGTAATTAGGTGTAAATGGTAAGCAGTTCAC
ATAAATGTTTTTGTAATTGGTATTAATTTTGCAGAGGCAAACCAAGTTTATGTGGTTTTTCTCTTCTTGC
TCTGTTAGTTTTCTAGTACAGGCATTTCCAAGCTTTTGGAGGAATTCTATTGCAAATGTTTGTTTATAGG
GCAATGTTTTGTGTTTTCTCTCAGAACAATGTTATGAATGGTTATGAGGTTCCCAAGCTGGTTTCCAAAT
GCCTCTTTGGCCCATACAGTAGGTGAAATGTGCAACGCGTGTGCCTGTGTCGGAAGGCCCGCCCAGCATC
CTCTGGCTCCAGGTGCCCCAGCGTGTGCAGCATGAGGGGGGCTGTGTGCGTGTGTGAACCTGTGCGCCTG
GGTCATCTCTGCATTTGTCTAGGTATATCTCTGTGTGTGCATCTATGTGTGTTTGTATCTGTGTGTCTGT
GTGTGTGTGTGTGTGTGTGTGTGTGTGTGTGTGTGTGGGGTCTGCATGACTGTGAGAGACTCCTGAGGCC
ACAGTGGCAGGTAGACATTCTCTGGTAAATACATTCCCCATTCTAGTGGCACGGGGCCTGGGCTGGAGAC
TTAGGGGCATCAGCTGGGAGGGGGAGCCTGGCACAGCAGCCGTTCCTAGGAGTGAGCACAAGCCACTCTG
AAGTGTGGTCTGAAGCCAGAGTCTAGGTTTCCTAAGAACATATGGCTTGCCTTTCTTTCTGATCAAGCAC
ATAATAATTTTTAAATGATCATTTATTGAGTACCTGCCACGTGCCAGGCCCTATGTTAAACACTTGACTC
AGATTCCCTCCTATCTTAAAATAGACTTATGAAATAGGGACTCTTTCTCACCCCGCTTTACAACTTGGGA
AACTGAGGCCCGAATCTCTCAGTGACAGAGCCAGGGTTTGAGGCAGGGACTGCTGGAGGCAGGGCCCATG
GTTCTGAGATGTGCTGCTCAGAACCCTGGGCTCTTCTCCCTCCCTCGGCCCCCTCCACAAATGGTTTGTT
TTTGTTGGGACAGACGATGAGGAGAAAGGCGGCCCTGTGGGAAATCTGAGAGCAGCGAGCTCCCTTCCTG
CTGAATTGGTTGGGGTGGTGGGGGACGAGCTCCAACAGCCGCTCCCAAGCCCTGAAGACCCTTTGGGGGA
CCTCCTGGTTTTGACCCAAGAGGGAGCTGGTTTAGGAAGTGGGGTTGGGGGACTGTGGCCATGGACGGTG
TGGGGAGAGGGTGGTCACAGGCCAGACAAGGACTCAGCCCCCCTGATTCCTGTGGTTCACTGATAGCCTA
GGCCCTCACCAACCCCTTCACCTTGGGGATCTGGGGCAGGTGAGCCTGCAGACCCACAGCAGGTGTATGT
TCTGCCCTCGGAGCTCCTGCAAACAGCCTTTCCACTGACGCAGTGCCTTGGGGGCTCTGCCAAGCGACCC
CTAGAATGGGGATTGTGGGGGGTCGCTCTAGGCACCGCAGCACTGTGCTGGGGATGTTGCAGCTGCCTGG
GAGTGACTTCACACAGTCCTCTCTGCCTCCAGGGTCACCCGGAACGGGGTTGCGGGGGACAAGCTGTGGG
CGGGGGGGACGGGCAGCAAGATACAACGCATGAACTCTGACCTCGTCTCTTTGAGAAATCAGACACATCT
ATATACGCATGGAACACTCAATGACCCCTGGGTGGGTGGTCGAGGAGAAGGGGACGGTGTTTTGGGGGAG
GAGAGATGTTGGGTTGAAGGACTGGGAGAGAGGAGCTGAGTGGGCCCTGTGGGAGAGGGTGGGAGCCAGG
GGTGTCATTCCGGGACAGCAGCCGGGCCTGGAATGATGCAGCTGGGAGAGGTGGGGGCACGGCCAGAGCA
GTGGGCGGAGGCTGCGTTGTAGCCAGCCCTGGTGAGACCAGGCCAGAGCGTATATTTTACCACGTGTGCA
AATCTTTGGCATCACCGGATTTTTAAAAATAATAGCTTTAAGATATAATTCACATTCCATACAGTTCACC
CATATAAGTGTACAGTTCGATGGTTTTAGTACATTCACAATCAACTTTTTAAAATTGTCATGAGGTGTCA
TAGGTTTTCATAGAGGGAGCCATGTGTGGGCAATCCTTGCTGATAGGGTGGGGTGGGAGGTAGAGATGCC
AGAGGCAGGTGGACCCGCTCCAGGGCATCTACCAGGTTATTGTGCCACAGACACAAGAGGTACAAAGGGC
CTGGAGGGAATAGGGGGACGAGAGAAGCCCAAGGACCAGGTACCTGCTGGATGTGAGGACCCAGGGGAAA
GGTGGGACCTGAGTTTTCTCCAGGGTTTGGGACTTAGGCAGCTGAAGGGAGGGGAATGTTACTTCCACAT
AGAGGGAAAAACCAGAAAATAGAGCCGGCTCTGAAAGAAGCATGTTCTGGAAGAGTAGGATTTTAGGGAC
TGCAAGTCATCCAGTTGGGATGAAAATAAATAGAGCTGGAGCCCCTGGCAGTAGGAGGCGTATGTTAACT
TATGCCCAGGTGTCTTAGAGGAAGAGCATTTAGAGAGAAGCTGAAGGTAGAACATGACCTTTGAGGAGCA
CCCACCGTGCAGAAGTGGGATCAAGACTCCCGGACAGCCCCAGAGACAAAGCTGCAGAATCTGAGGCTGG
AGACCCAGAAGCAAGGAGCCAGATGGAGTCATGGCTGGGAGAGAACACACAGAAGGCAGTTGCTGGTGAG
CTCCAGAAGCAGGGGTCTCCTTTCACTCCAGGGCCCGGCTCCACAATGGCACTCAAGAAATGGCTGTTGA
GTGAGTGCTTGAAGCAGGGAGTGATTAGCTGGCCAGGATGTTGAATTGAGCTCAAGGGCCATGAGGATGG
AAAAAAGCCCTTTGGGGCCGCGGGTGACTGTATCAGTCAGGGTCCATTCAGAAGACAGAAACCACATGGT
GATTGGAACAAGGAAAGTTTCACATAACGAATTATTAACTCTAATTGGATTGGAATAATGAGGAATTGGC
TAGTAAGAAGTAAAAATAACTCCAAAGAATACAAAAATAATAGATATGAGAACAGCCCCTACCTCTAGGG
TTGCCACAGAGTGCCCAAGGAAGAGGCCCCGCAGGGTTGAGATCCACACCTTGATGGAGAGGGTGTGGCG
ATGGCTCCCTGGATGGCAACGTCTCTGTAATGCCATGTTGGCAGAACTTGCTGGAAATCCACCCTCTGGA
ATTTACTGGAAATCCATCTTTTTGGGTCCCAAGGAAAGTTGTTTGTGGGAGGTATCTTGCTGGAAGCACA
CCGCTGCTACGTTTTCCCAGGGGAGTGCTGGGAGAAGCTCCTGGCTGTTGGGTACTCCTGACTGACGTGT
TGCTGGAGCCAGGTGCTGGAGAAGCTGTCTGCATTGCAACAGCCTGGCACTCCTGCAGGAGCTGGGCACT
GGAAAGCTGCATGCTGGAGAAGCTGCAGCAGCACAGGAGCTGGGTGCTGGGAAGCTTCATATCTGGCAAG
ACCCTGATGCTGGGAAGCGCCCATGCAGCAAGATCTGGCTGCTAGAGAAGTTGTATACAATGCAGGAACC
CAACAAGAGCACACTGGGTCTAGAAAGGAAACCCACCTCCTCCTTCAATGCCTCTTCAGTGCCCTCCACA
GTTGTGCCAGCTGACAAAGGAAAAGTAGTTAAAGGGCCCAGCTCCATCTTCACAGCACAGGCAATGATGG
GCGAATAAACTGATAACTGGCACAGTTTACCCCTTTGGCTGCACAGCTTCCATTTGCACCTTTCTACAAC
ATCTGAACTCCCATACAAAAACAAACAACTGGATGTCTACCTATCAAGATAGTTATCCTTCTTATGAAAT
TCTCACCCTTTTGCCCAGAGAGCCCCAACCATCATTCTACCAATCCCTGGCTACAATAACTTCTCAAAGT
CAGTCACAGTCTCACTGAATATTCTGTTACCTAAAGACTACATTATAAAGCTAACTGTCAACAACTTGTA
TAGAAAAGAATGGGAAGGAGAAAAAAAGAAGAAAATGGTTACCATATTGCAAACACACCACACACACACA
CACACACACACACACACACACACACACACACATATATATCAAGTTAATAGAAAATGTGCAAAACCACTAG
AGGCCTGGTTTCTGCATTTGTTCACAAGGCTACACTTTACACTTGCTACCTGGCTTCATTGTCTTCTTAT
CCCATTCTGTGTTTCCTCCACACTCAGCCAGAGCCTTAGCAGGTCATGCTTCTTTTACTTGGTGGAGTAA
CCCAAACCCTCATTCCCAAAGGACCTGAATCCTTAGTTATCCCGCTTTTGTTGTTGTTACCATAACTTTC
CATTCAGCCTTCCTTTGGGCATGGACGTTCTTAAGAGGTGCCTGAGAGAATTCGCTGGGTTCCAGACACA
GTCTTGCTTGGCTCTGTGGCTGATCAGCAATCCAGTTTTCTCCTTGGTAACTAGGATCAATCACTCCAGA
CAGTGGCATAACTCCTTTTTTCCACTGTCAGTTCAGTGGCGTAAGGAGTTCGAGAAAGCCAAGTGGGTGT
GTGTGTCTTCTCTCCCAGGGAAAGCATTATAATATGTCCCTGGTGGGAGCACTCTCCCCCGAGGACGAGG
TCAGCATCCCCAGAAGGTTTTAGAGTTGTGGGGTGGGGAAACAGGGCCACGCGCAGGGGACTAAGTGATA
GAGGCTCTGGAGCTGGCAGCTGTGGACCATGCTGTAGAGAAGGTTGGAAAAGTAGAGAAACATGCAGATG
ACCGTGGGCCGCTTCCGCTGTGGATGATAAAATCAGACCCATGCTGGTCACTGCCAGACCCCATCCAGGA
CCATGTGTCTGATTCTCACATGGACTCTGTGCCACCCTGTGCCCCTGACTCCCAGGCCGTGGGAAGCCCA
TCTGACCCACAGCACGTGTGACCCCTAGACAGGGCTTCTCCTGGCTTGCAGCACTTGGAAATCGGTGCTG
CCTTACGGAAGTCTGTGTTGGCTCATTTTGGCTCCTGAGAACAACTTCAGCTCCTTAACAGCATCTATTT
TAGGTATAAATCCCCCCCCCGCTCACTTTTGGGGCTGTGTCATGGCAACCATTAGTGTGAGCAAAGCAGT
CTGACTCAGACTCCCATGTGGCCGCCGTAGTGGCAGCAGGTTTGGTGTGATCATAAGTGCTCCTGCTGGT
GGTGCCAGTGCTGGTGGTGCCAGTGCTGGTGGTGCCATGGCCTCGAGAGTGGTGTGAGAGCCACAAGCCG
TCTTATTGTGACTCTAGAGCCAGATAAATCAGCTTTGCAGAGGGCAGGACTGCTGGCCTGTCACCAGTGG
CTTTTGGTATTCTATGGACAGCTGGTAGTTTGGGTGCAAGTAATGTTTCTGAAAGTCAGGGCAATGGAAT
GGTGATGGCCATCTCACTGGAAAATGGCACCAAGACCCCCCTCCCTCTCCCTCCTCCTTATCCATCCCCA
GTCTTATGACCAAGAGAGGTTGTGACAGGAGGGGGAAGAGTTGACAGATAGACATCTCCGTCTAGGAGGT
CTCCTCCACTTTCTCCTGGCCCCTAGCTGACCCCTACCCTAGCAGATGATTGGGCAGGGGGTGGGGGTGG
GGCACAAGATACTCTGCTGCCTTCTGTCACCCACATTTGCACCTCCTCCTAAGCTCAAATAGGAAATTTA
GCCATGGGACTGTTATCTGTGGGCCAGGGTCACCCCAGAGGCACAAACATCGATCATGGCGCTGGCTCTG
AGATGTGTGTTTAGCATGCTCATTCATGACCAAGAACTGCCACATTTGCGAAGGGTTCTGAGTTACACGG
ACCTGGATTTATAATCCTGCCCTGGGGCTTTCTAGCTGTGCTACCCTAGAGAGGTATTAAACCTCTTTAA
GCCTCCATGTCTTCTATAAAATGGGGATAACAATAGGACCTGCTTCATAGGATTGTTTTGAGGATTGAGG
ATAGTGCATGTTCCTTAGCACAGTGCTTGGCACATGGTAGGTGCTCAGGAAATAGCTGCATGAATGAATG
AGTGAATGACCACTGCATTGGGCAGGGCATTGAATACTGTGATTAGCATGCTCCAGTGCCCTCCTTCCCC
GCTCCCACTGCAGACAAGCCTGACCAGGAGAGAGAGACTGACTGGAAACTTTGTTTTCTGGCCAGTCCGT
GCCCAAAGTTAATATAGATCTGCAGCAGTCACATGCTGATTGTGAGAGAGGGAGTGGAGAGCTGTGAGCT
TCTGAGTGGGGTGGACCTGGACTGTGCCTGGCTCCACCCCTTACCCACTGTGTGACCTTGGGCAGTTTTA
TTAATCTCTCCATACCTCATTATTCTCATCTGGAAAATGGGGATAAAAATAGTACCCACTTCAAAGGGTT
GGGGTGAGGTGTGAGGATTAATGAGATGTTGACATTGTAAAGCTCTGCTCACGGGGCCTGGCATGTCGGA
GGAGCTTAAGTACAAGCTCCTGCCTGAGCGAAGGCCACTTAGAGGAACTCCAGGGATTTCAAGAAAGGAG
AGAAATCTGCGGGCTGGTTGAATCTGAGAGTAATCATCGTTAAGATTTATTGAGTACTTTCTCTGTGCCA
GGCGCTCTTGGAAGCATTTTTAGGGCATTCCATTTTAATTTTCACAGCAGCGCTGTGAGGTGGAAAGTTC
TTTCTCCCACTCGACAGCTGAGAAAACTGAGGCACAGTTTCGCCTCAGGCCACACAGCTGGTAAGAAATA
AGGCCAGTGTTGGAAACCCCGCAGTTGGCCTCCCAGGGAGCCCTGGAGGGGACTGAGATTAGCCCTTAGG
TCAGGAGGGTGTCAACCACAAGTCATAGACTCATACCAAAGTAGTGAAAGCTCACAGTCCCCGGAAGTTC
AGAGACGTCCCTGCCTCCAGGAACAGCAAGACCAGGGGCCCAGACAGTGACTACAACGCTTTATCCCTCT
GTCTTCATCTCTGAGCTCTTCGTTCTCAGGCAGGCTCACCCACATGGCGGGAAGACAGCAGCTGGCAGCC
CCTGACTCACATCGACCATGTTAGCATCCTTCCATCGCATCGATAAAAAGGTTCCAGGGAGGCCTCTGAT
TGGCTCTGCCTGGTCACGTGCTCACCACTTGCCTGGGGTGAGGGTTGAGGTCAGCACAGGCACAACGAAA
ACCTTTTGACAAGCGTTTCCCACCGGGAAGGAGGGGAAGGCATCTAGGTAGAGCAGAACAACCAGAGTCC
ACTAGAGGAAAAGGAGAATGCAAGCAGAGGGAGGGAGAAGGACCTGCTACAGAAATGGTCAGATATTTTT
CAGCCATTCAGACAGAGAACTTGAGACGGAGAAGAAGGGGCCCTTAGGGTGAGAATATTGGGATCGTGTT
TGGTGGAGTGAGGATTTAGGTTTGTGGGCCGTGACAGTTTTGGTTTTGGAAGAGGATAATTATGTGCAAG
CCCTTGTTGAGGAGAGACTGTGGGGTGGCAGGGGGGCCAGCAGTCCTCAATCTGAAGATGGAGAGGAGGC
TTGGGTGGTCATCTCGGGGGTGCTGATGAGGCTGGGGTTGGTGAGGAAGTGGGGGCGGCAGTGGGGACAG
TGTGGTGCAGGGGAGCAGTCCTGAGGCTTCAGGAGATGGTGGAGATGTAGCTCTTTGGTCTGAGAAGGGG
CTGAGTGAGCGGGGGTTGGGGTTACGGATGCAGGCAGCACTGACTTGCGCTGCCCATGGGCTGGACGCAG
CGGGAATGATGTGTTTGGAGGATTAAACATTTCGTGAGGGTGGGGGGTGGAGAGGGAGGTACCAGTGTTG
TAGCTGGTGATGGAACTCTGTGTGGGTGTGAGGAGCTGATGATGGTTATTCTGTGGGACGTGATGATTTC
ATAGAACAACAGAGGTGGAAAGGCCTCAGCTACACTCTTATTTATAGAGAAGGAAATGGAGGCCAAACGC
AACCGATGGCTGCAGTTGGGACGTAAGCCCTTTCTCCCATCCCCACTCTCTCTGTTTAAAAATTCATTTT
TATTTACTCACAGTAGTACATGCATATAATTTAAGAAGTCGAATAGTACTAAAACCTCCTAACAAAATGC
AGTGATTCCTTGCCTACTCCTACCCTTATCCTCTTCTCAGAGGCAACCGCTTTCGATTCTCTTGGCTGTT
TCTTCCAGCAATTTTCATCTCATATTTCTGAGTAAAGTGCATATTTTTGATTCATCTATTTAGACATTTT
CCATTGATTTCCTATTTAGTAGATCCTCTTACATGTCCAACCTTTCTGCTCTTCAGCCATCCTCCTTAAA
TATTGACAGTACTCTTCTTATATTTTTTCAAAAAATTGTTTGTTGTTGATTGTTTTGAGACAGGATCTCG
ATCTTTGCCCAGGCTGGAGTGTGGTGGCACAATCACAGCTCCCTGCAGCCTCGACCTCCTGGGCTCGAGC
GATCCTCCCACATCAGCCTCTCGAGTAGCTGGGACTACAGGCATGTGCCACCACACTCGGCTAATTTTGT
AAATTTTTGTAGAGACAGGATCTCCCTGTGTCATCAGACTGGTCCCAAGTGATCCTCCTGTCTTGGCCTC
TCAGAGTGCTGGGATTACAGGCATGAGCCATTGTGTCCAGCCTCAAAAATGTTTTGTGCATTTGGTAGCC
AGAATAATGGCCCCCCTAAGATGGCCAGGTCCTAATCCCCTGGACCTGTGAATATGTCACCTTACATGGC
AAAAGGGACTTTGCAGATGTGATTAAATTAATGACCTAGAGATGAGGGGATTATCCTGGATTATCTGGGT
GGGCCCAGTGTAATCAGAAGGGTCATTAGAAGTGAAAGATGGAAGCAGAAGAGTAGTCAGAGCAGGAGAT
GTTGATGAGGCAGAGAGTACTGTGATGGGAAAAAGACTGGCCATCGCTGGCTTTGGAGTTGGAAGGGGAC
CACGTGGCAAGGAATGAGGGCAGCCTCTAGATGTTGAAAATGTCAAGGAAATGGATTCTCCTTAGAGTTT
CCAGAAAAGAATGCAGCCCTGCCAACATCTTGAGTTTAGCCTAGTAAGGAGAATCTTTTTTTTTTTTTAC
TACTGACTTGCAGAATTGCAAGATAATTTTTTTTTTTGAGACGGAGTTTCGCTTTTGTTGCCCAGGGTGG
AGTACAATGGTGCCATCTCCGCTCACCGCAACCTCCGCCTCCCAGGTTCAAGTGATTCTCCTGCCTCAGC
CTTCCCGAGTAGCTGGGATTACAGGAATGCACCATCATGCCTGGCTAATTTTTGTATTTTTAGTAGAGAC
GGAGTTTCTCCATGTTGGTCAGGCTGGTCTTGAACTTCCGACCTCAGGTGATCCACCCTCCTCGGCCTCC
CAAAGTGCTGGGATTACAGGTGTGAGCCACCGCGCCCGGCCGATAATGTGTTGTTTTAAGGCATTAATTT
TGTGGTACATACAAAAGTAATTTGTGTGTACATACATGCATATATACATATATATGTACACACACATATA
TTTGTTAAACCTACACACAGTGTTTATTTTATTATTTCGATAAAACTATTATTCACAGCTGAGCCTTGTG
GTGCACTGTAATAGTTTCTTTTCGTTTTTTTTTCCCTGAAGTTAATAATTACCTTGTTTCTCATCTGCCT
TATTTTTTTGTACCTGTTGCTAATTCCTACCACAACTTCTAATATAATTTTATTTTTCTTTAACAATACT
GTTACCTTAGGACTTCTTTTTCTTTTTTTCTTTTTAACTGACACATAATAATTGTGCATCTCTATGGGGT
ACAATGTGATGTTTCAATACATGCATACATTGTGTAATGATCAAAACAGGATAGTTGGTATATCCATCAC
CTCCAGCATTTATCACTTCTTTGTGGTGGGCCTACCACACTTTCCAATAGCCATCAGTATTGCTTTCCAC
ACGGGCAGTCACAGCAGGTAAAAGATGTTTCAGTTCCATTTTTTAAGTTATCTATCCTCTTTCACTCTGG
GCTAGTTGCTTTCTAGGCACATTGTAGCTGATGCTCTGTGACTTCCTTTTGTGGTCATCTTTGGAATTCC
CATTACTTTCCGGTTGGAATCTCTGTTTTCTGGCTTTCGTGTCCTCCTCATGCTTGGCTCACTCCCCTCA
TTAGAGCACATCTTCCAGGACACACATCTGTCCATATCAAAATCCATTCATGGCTGGGCACAGTGGCTCA
CGCCTCTAATCCCAGGACTTTGGGAGGCCGAGGCGGGTGGATCACCTGAGGTTAGGAGTTTGAGACCAGC
CTGGCCAACATGGCAAAATCCCATCTCTACTAAAAATGCAAAAATTAGCCGGGCATGGCGGCGGGTGCCT
GTAATCCCAGCTACTCGTGAGGCTGAGGCAAGAGAATTGCTTGAACCCACAAGGCAGAGGTTGTGGTGAG
CCGAGAATGCGCCACTGCACTCCAGCCTGGGCTAGAGAGCAAGGGTCCATCTCAAAAAAAAAAAAAAGAA
AGAAAGAAAAAATCTATTCAGTTCCTGGGTTTAGATAGCAGAGTATGGCCACGAGCGATACCTGTGTAGT
TTTGTCAGGTAAAAAGACAGTTCGACAGCAGAGGGCATGGTGATGACGGCACTCGTGCCCCAGTCTCAGT
GGGAAGGCTGGCCCTCTCTGGCTCATGCCAGGTGACTGGCATGCGGCCTCTTTTCCCCTTCATCCTGGAG
CCTCCCTTCCCATCTCTCCTGTACTGGATCTTATGCGTATTAGAGGACATCTTCCTGTCCTTCCTGGGAA
AGGAGCGTGAGGCCTCATATGTCTGAAGATGTCTTTACTAGGGCCCAGCACGGTGGCTTACGCCTGTAAT
CCCAGCACTTTGGGATGCTGAGGTGGGAGGATCACTTGAGCCCAGGAGTTCAAGATCAGCCTGGGCAACA
TAGTGAGACCCCATCTCTATTTAAGAAAAAAAAAAGACCCCGGGCGTGGTGGCTCATGCCTGTAATCCCA
GCACTTTGGGAGGCTAAGGCGGGCGGATCACCAGGTCAGGAGCTCGAGACCATCCTGGCTAACATGGTGA
AACCCCGTCTCTACTAGAAATACAAAAAATTAGCCGGGTGTGGTGGTGGGCACCTGTAGTACCAGCTACT
TGGGAGGCTGAGGCAGGAGAATGGCATGAACCCGGGAGGTGGAGGTTGCAGTGAGCCGAGATCCCACCAC
TGTACTCCAGCCTGGGCGACAGAGCAAGACTCCGTCTCAAAAAAAAAAAAAAAGAAAGAAAGAAAAGAAA
AGGGCCAGGCGTGGTGGCTCACACCTGTAATCCTAACACTTTGGGAGGCCGAGGCAGGCAGATCACGAGG
TCAGGAGATTGAGACCATCCTGGCTAACACGGTGAAACCCTGTCTCTACTAAAAATAGAAAAAAAATTAG
TCAGGCATGGTGGTGGGCGCCTGTAGTCCCGGCTACTTGGGAGGCTGAGGCTGGAGAATGGCATGAACCC
CTGAGGCAGAGCTTGCAGTGAGCCGAGATTGCACCACTGCACTCTAGCCTGGGCGACAGAGCGAGACTCC
ATCTCAAAAAAAAAAAAAAAAAGAAAAGAAAAAAGAAAATGTCTTTATTTTACTCTCACACTTAAATGAT
AATTTGGCTGAAAATAATTCTAATTGGAAGATTGTTTAAGATTTGGTTTATTCTTTAAGAATTTTGGCTG
GGCACGGTGGCTCACACCTGTAATTCCAGCACTTTGGGAGGCCAAGGTGGGTGGATCACTTGAGGTCAGG
AGTTCAAGACCAGCCTGGCCAACATGGCGAAAGCTGTCTCTACTAAAAATACAAGAATTAGCCGGGAGTG
GTGACGGGCGCCTGTGGTCCCAGCTACTCGGGAGGCTGAGAAATGAGAATCACTTGAACCCAGGAGGCGG
AGGTTGCAGTGAGTGGAGATCATGCCACTTCACTCCAGCCTGGGCAACAGAGAAAGACTCCGTCTCAGAA
AACAAAAACAAACAAAAAAACCCAACTGATCGGAAGCTCTGAGAATGATAGTGGACTTGTTGGTTTTCAA
GTCTACATAGGCTGACCTGAATAAGTCATTTTGACCCCATTTTGGTACTGATGTTGGTATTGGCATCCTT
AAACCTTTCCTCTTAGACTCATTCGATTCTCCAGAGAAGGAAATGGTTGTCTGGCTGCCAGTATTCTTAG
AGCCCAATCGGGGAATAGCCTGAGAGACTGGACATCAGCATTTAAAATAACCTTCCCGGCCGGCGCGGTG
GCTCATGCCTGTAATCCCAGCACTTTGGGAGGCTGACGTGGGCAGATCACCTGAGATTGGGAGTTCGAGA
CCAGCCTGACCAACATGGAGAAACCCCATCTCTACTAAAAGTACAAAATTAGCCAGGCTTGGTGGTACAT
GCCTGTAATCCCAGCTACTCGGGAGGCTGAGGCAGGAGAATCGCTTGAACCCGCGAGGCGGAGGTTGCGG
TGAGCCGAGATCGCGGCATTGCACTCCAGCCTGGGCAACAGGAGTGAAACTCCATCTCAGAAAATAAATA
AATAAATAACCTTCCTCTCCTTGGTTTGATATATCATCTTTGTCTTCAGCTGTGCCAAGTGACTCTGTGC
AAAGACCCCCTATTTTATGCCCTCCAGTGACTACAGATAAACCTTCAAACTTGTAGAAGAGGGTCTAGGA
ATTCACTACTCTCACTCAGCCTCTCGTTTCTCACACAAGCATCCTCTTCAGCCCTAGTGCCAGAGTACCT
GGTGCTGTCATATAAGCATCTGTTGAAGACTGTGTTATATATCAGCGTTCACACGCCTGTCTGAAATTTG
GCTGTCACAAGTCTGCTTTCCAGATTCCACAATTTTGTTGGTGGTATTCCTTCTCTTGTTCAGCCTAATC
TTGTGATTTTATGTCTTAAAAAATCTTTTGGCCAGGTGTGGTGGCTCATGCTTGGAATCCCAACACTTTG
GGAGGCTGAGGTGGGAGGATCCTTTGAAGCCATGTGTTCAAGACCAGCCTGGGCAACATAGGGAGACCCT
GTTTCTACAAAAAAAAAAAAAAAAAGCCAGGTGTGGTGGTACATGGCTGTAGTCCCACCTACTCAGGAGG
ATGAGGTGGGAGGATCACTTGCACTTGAGCCCAGGAGTTTGAGGCTGCATTGAGCTATGATACTACCACT
GCATCCAGCCTGGGTGACAGAGCAAGAACCTGCTCTAAACAAACAAACAAAAAATGTTTCTATTGCATTG
AGTTTAGAAGGTAGTGAGCCTTGGTATGCATATTCAAAGCTTTCAGTTCTTTTTCCACAACAGTTTTATA
GTTGACGCAGTGGAGATGGTGATCATATCAGCTGTGATACGAAGATGTTTTCATGGTGGGATTCCTTGAA
ATTTTTTAAGTGGCTGTTAACAAACATTATTCATGGTCACTGTGGACTCTAAAAGGAGGAAATAAAAGCT
GCTTTTCATGGTGGTAGGGTTCTTGGCAAGGGTTTTTTGGTGGCAGTGGTGGTGGCTGTGCAGCCCTTGG
CCCAGGTGGGAAGAACGCTTTGGTATCGGAAGCAGCCAGGGCTGTGCGGTGGAAGGACCGATGTGGTGGA
CACCCGCTGCTGTGTTGGGACTGTGGGCCCAGGAGAGCCCACTCCCCATGGCTGGGCTGCCTGAAGGAAG
AATGAAGCGTCTACGGGTAGGTGGGCAATGCTATGTCAAGGTCTGGGACCTTGGCCACCAACTGGGGCCC
CCTAGGGTTGTGTAGTACATGGCCCGCCCAGACACATACTGGAGGATTTTAGGTGATGGACACGATTTTG
AGGGACTGCTACTGACAGGGCTGGTCCATTGAGCAACACGAAAGGGTTGACCCAGGTCAGCTGCATTTTC
TGTGAGGACAGTTTCTGCAGCTCAGATGTCAGAGTCTTGGGTGGAGGAAGGGGAGGAGGAAGAGAATCTT
CCTTTCCTTTCCTGTCTGGGGTGTCTGCCCTTGGGGCCAATGCCCTGTGAAGTTCTGCTTTTCCCTCCAG
CTTCCTTCCTCTCCAGAGCTTCTCTGAACCTCGCCACACCCGTGGCGGCACAGATGCCCTCTGGGTGTCC
CTAAGCCTAGGGCAGCATCAAGACAACCCTCCCGCCCCGCTTCCTTTGTGCCTGGGTCTCCCCCGACTCC
ATTTGGGGTGCCCTGTCCTTGTCAGCCCCTTGTGTGTCCACTGTGCCTGTGGGGCCGCAACAGCTTCCTT
GGCCGGTCAGAGCTATTAATAGAAGGGGTCCTGCTCTCCCATTTCATTGGTGGGGAAACTGAGGGCCGGG
GAAGCCTCCGTCAGGAGTAGATCCCACCCCTGCGGACTTTGGGCTCAACGTTCTTTCCAATCAGACTCTG
CCCCTCTTAGCTTCTGACCCTTTCGAAGGTTTTTGTTTTAGTGTGGCTGGAGATGTCTGTGGTAACAAAT
GGGGAGTGAGGCTTGACAGGGAATCACTGGCTGCTAAGGGCTGGCAGCCGAGTGCTTCTGGGTTGGACCA
GTTCTCTTTTGGGATCTTCAGAGTTGCTTCTGTGGTGTGTGTTGGGGGACGTGAACCCAACCCCTCAGAA
CTTTAGTGTCAGAGAGATCTGGGTTCATGCCTCTGATGGGTCCTGGCCTTGTTGATCTTACCGTCACCTG
TAAACTGGGCACGGCACAGCAGCTGCCCAGATGCAGAGCACGAGCCAGGCCCTGAGTGGCATGTGGCCGA
TGGAGCTGCTCTCCGCTGCCGTCCACCCTCTCGGGACCCGGGCAGCAAACCTTCCTTAAGCCCAGCACTG
CGCTGTGCCTAGGGGTATGGGGAATGGACGCAGTGCACCTGGGCTGCCTCAGGGCTCAGCTGTGGGGCAG
GTCCGCTTCAAGCCTTTCTGGGTTGACCTGATGGAGCTCTTCCTGTCCTGGCCCTAAGAAGCTCCTCCTG
CCAGCTCCTTCCTCCCACTACCCCCTGGGGCACAGCCAGGGCTGGGGCACCCACTCCCAGGCAAGAATGC
ACCCCTGGAGAAAGGGGCTAGGCCTGTAGGAGAGCCAAGCCTGTGCTTTCTCCCGGTCCTGGTTTTCCAG
GGAGCCCTGACTGGAGGGGGATGGGAGGAGAAGCCCAGGAGGGAGGGGGAGCTGGGGAAGCAGAACCAGG
AACTGGAAGGGGAAGGGTGTGGGTGGGCCCAGATCCAGCCTGCCTCCCGCTCTCCCTTGCCTGCCAATCC
CCTCCTGGGCCCTGGCACACTCAAGGCAGGGGCTTGGCGGCCGCCTCCTCCGGGGGTGGGGAGCAGGGGA
GCTCTTGTGTGTCTCTGAGTGAGGGGGCTGAGACAGCAACACCCCCACCCCAGCTGTGGCCTGAGCTGCA
GGCTTTGTTATTTCTCTGGCTTGAGGCCTACACCTGGAGCAGAGGGAAGAAAAGCCCCTAAACTCAGCCG
TGTGGGTTCAGAGGACCCCTCAAGCCCACCAACTCTGCAGGCAAATGAGGCTGGTTCATGGGGTGGGGGA
GAGAGCAGCCTCACTCCTGCCTCAGTGTGGCCGTGGCCTGTGGCCTTGGCCGTGGCCGTGGCCTCTCCCT
CCTAGGCCAGAGCCAACCAGGCCTGCAGCCTGAGCTCAGACCTCAGCTTTCTCTGGGCCCAGGCACCGGT
CCTGAAGTGAGGTGACATTCATTTCACTACATCCTGTGACTGCACTCACTTCCTCCACGTGGCGGGAGGT
GTGGGGGTGGGGGTGAGGACAGATTAGAAGGAAAACGTAGCTGGGCTAGGGGCCTCTGGGGGAGGCTGGG
ATTTCCCAGGGGCTGGTTGGGGTGGGAGGGAGGATGCGGGAAGCTTTCAAAGGAGAGAGAGGCTGTGAAT
GAAGAGAAGGGAGGAGGCGGAGGCAGCTGGAGGTCCCAGAAGTGGCACCCATTGGCCGCAGCGACACGGA
GGGGAGACTGCTGCCACTCCAAGGCTCTCAGAGGCTGCTGGGGTAGGAGGTGGCCCTGAAGGGAGCCTGC
CTCGGTTTCTCTGCTCTCTGGGGTGCTGGTGAGGCCAGAAGGAGGAGCAACACCCCAGGCCTCACAGGCC
AGGTGTGGCCCTGGGCTGAGGGCCTGACCCAGGGAGGGTGGGGTGCTCCGCCCCACTGATCCTCTTCTCT
CCCCCACAGGTGGAGCCATCGAAGCCCCCACCCACAGGCTGACAGAGGCACCGTTCACCAGAGGGCTCAA
CACCGGGATCTATGTTTAAGTTTTAACTCTCGCCTCCAAAGACCACGATAATTCCTTCCCCAAAGCCCAG
CAGCCCCCCAGCCCCGCGCAGCCCCAGCCTGCCTCCCGGCGCCCAGATGCCCGCCATGCCCTCCAGCGGC
CCCGGGGACACCAGCAGCTCTGCTGCGGAGCGGGAGGAGGACCGAAAGGTGAGCAGCGGGCTCTGCTGGG
GACCCTCTGGCCTGGGAGAGGGAGAGCCCTGTCCCTATGTGATGACAGCCGGCTCCTCATACTGAGTGCA
TGACCGCATCACTCAGTGCTCACGACAGGGTGTGGGACCATCACATCCCTGTTTGGGTGATGAGGAAACG
GAGGCTTGCAGAGGTTCAATTCTGGTGATGGAGCAGGCATTTGGTTCCAGGACCCAGGCTTTTAACCACC
ATGGCATTCGCACAGTGGCTCAAAGCCCAGGTCTGTGCCATTGTGAGGGTTCGGCCTCACAGAGAGTCCC
AGAACCTGGATTCCTTCTCGGACCCGCCTCTTACTAGCCGTGGGCCTTCACGTGAATCACTTTCTCTCTC
AGCTGCCTCTGTTTCCTTAAGGTGTTCAGTAGCTTCTAGGGGACCAAAGACAAGTTCTGGGGTGGGGCAC
GGCAGACACGAGGCCGCGGGGGCCCACCCTCCCACACAGACGGAGGCACAGGCTGGGCTCAGTGTGGCGC
CAGCACACTTTGGAGATGGTGATTTCCTGTCCCCGCCCCCCACCCCAGACTCCAGGTGAATGTCGCTGGG
TTATCTTTGGTATGGCTTTTGGCCACGGAGCTGGTGACCTCCATGTTTTCAGAATCTAGTTGGGGGTGGC
CTCTCCACCCTTCTGCTGAACTCCAGGTTGTGCCTGAATCAGTCAGAGGAGATTACATCAGACTGATGGG
AGTCCCTGCTGGGTCTCCACAGGCTGTCTTGGGACCTTACCAAGCTGTGTCCCCATGATAGGAAAAGCTG
CCCCTTAACCCCCAGCACTGGACTGTGTTGCCCTCTGTCAGTGGAATGGTGAACTCACTGCCACCCTCCA
GGGGATCTTATGATGGCCATAGGCCTTCCTGGCATGTCTGTCTGCTGTGCCTCTGTCCACAGTTCCCCCT
GCCTGCTCCCCTTCCCCTGAGTAGCGCTGGGGGTTGTGCTGCTTGGTGGTGTCTGGGAGTAGCTGGGCAT
GCTCATGGGGGCACCAGTTGGTTCTTTTGGAACAAAGGTGGACACCTGGCACCAGCTTTGGGGGCCCTGG
GCACTCTGGGGCTAGAGGCAGCCAGCACCCTACCTGAAGGGTGTCAGCTCTGAGACCTCCCTCTTGCGTG
CTCAGCTCCTTCCTGGGTTTACCATTCTTCAGAGTGTGTTTCTGTTTCCTAGTTTTCCATCAATGGTTCT
ATTTGGTCCTTGGGGACAACTCTTGAAACAAATGGGGCAGGGACAGATGGCCCCAGTTTACAGATGAGGA
AGCTGAGCCCCGCTGGGCAGTGGCAGGTCCAGATGACAGCCTGTTCTCCTGTCTCCCAGGTCCGAGCCCT
TCCCACAGCCCTACCTGCATAGGTCCTGCTAGGGACACATGTACACATCCCAGGAGTGCACATGACGCCC
AGGGACCTGCCTTCCCAGCCCAGCACCCTGTAGAGCACCGTGGGGGTGTGTGCACCCAGTCAGCCAGAGC
AGGTCAGAAAGCCAGCATGCGCTGCGGCCCACTGGGCAGGACGCCCGGGGCGACCGATCAGGCTCCTCCC
TTCTGAATCCATGGGAAAAGAACTGATAGTCCTGGATACCTGCGGGCCCCTAGGCTGCCCCCGCTCAGGA
CTCAAGCAGCATGAAGACCCAAGCCCTTTGGCCGTAGAGTTGGGGACACCTGCCGCTGTCTGTGCTCTGG
AGTGGCAACTGTGGCCATGGCTGACTCCCTCCCCTTCTTGGGCCTGGCTTGCTTTCTCTGTCGGATAAGG
TGACCCCGAGTGTCGCCCCACTCTGGGTCCGATGTCCTGACAGTTGCTGACGCAGCTCCCCATCAATGCA
CTGGCAGTCACTGGTCAGCTGCTCCCCACAGGTGGGGAGGGTGGGGACAGCTGGGACAAACTCCTCCTGT
TACTGGACTGTACTCCCACGCCGCTGCTGCTGCTCTGGGCTGCGGAGCCCCAGGCCACCACCAATGGACG
GGAGCAGGTCAGGATCTGCATTATAATAGCTCAGGACTGAGAAGCAACGCTGGACCAGGAGTCAGAAATC
CCAGGTCCTAGTCCTGAGTATGCCACTTACTGGAGATGTGACTTCAGGCCATCTGGGCTTGTGTTTGCTC
AGCTGGAAAATGGGGATAGTGACTCCAGCCTTGCCTGTTTTAAAGGGTGGTTGTGGAAGTCAGATAAGGT
CATGGGCATGGAAGTGACCTGCAGACAGCAAAGTCTGGTCCAATTATGAGGGATTACGATTGCTATTATT
ATCTACCCATAGTAACTGCTCTGGCTCAAGCGGCAACAAACACACACCCAGCCCTAGATCCCGGTGCTGA
TAGAGTGCACACTCACACCCCAGCAGGAGGGGTCATGCCAAAAGATGGCAGTGATCTGGATAGATAAGGG
CTGGGCCATAGCCAGGCCTCCTCCCCAGCCGCCCACACCTACACCACGCATGCTGGCTCACCTGGCACAC
TTCCCCGGCACCTCTGGGCTGGCTCCAGCACCTCCAGGGAGCCGCCCAGGGAGGGTGGGGAGAGACACTG
ACAGCCACCACAGCAGCCCATGAAAGCCAGGCCTGCGGCAGGGGTGCTGGTAGCTGCAGAGGGGAAGACA
CAGGCTTGGTCTGCAGCCCAGGGAGCCTGGCACAGGTGCCAGGGGCACTGTGCACAAAGGAACTGTTTGG
CAAGCCAGATGGAAGGGGTGTGGCCAACCAATGGCCCTTCCACTGCCTGGCCCCATGTGGCCCTCTAGCC
TCTGCCCACCGCCGCCCCCCCTCCCCGCCCCCGCCCCCAGCCCCGCCTCCATGCACCTTTCACTCCAGCC
CCACTCCCATACTTCCTGGAGTCTCCAGGTCTTTGTTTATGCTGCTCCCTCTGCCGCATGGGCCCTTTCC
CTAATCTCTTCTCCTCCCTGGTCAAAGCCTCAAAGCCCAGGCTGTAGTTGGCCTTGTATTATAGTTTGCA
GGGGACCAGTGGTTCTCAAAGTGGGATCCCCAAATGAGCAACTTCAGCATCACCTGGGAAGGCAAAGCTC
AGGCCCCATTCCAGACCTGCTGAGCCAGAACTCGGGGGATGGGACCCAGCCGGGCACCTTTAACCATCCC
TCCAGGGGATACTGTTGCGCACAGATGTTGAGAACTATTGGTGTAGATAACTTCTGCCCACTGCTAGCCT
GGAGCGCAGGGTCACGGCCCTTTTCTTCCATGTGAATCCCCCGGACCTGGAATAGCACAGAGTGGGTGCT
TGGGAAATATTTTACGGATAGAACGGAAAGAAAAATGAGAGTAGCCAAGACCCTCCCACAGGAGAATAAT
CCCGGAGCCCAGGATCAGGGAGGCGGAGTGCAGAGCTGTGGGTGCTGGGCTGAGGCAGCCATGCTGTGGC
TGGGAGTGTACAGAGTGGCCTGTGTGTGTCCCCCTGGCACCTGCCGCCTGCCCTACCCGCCATCCATATG
GTGGGCAGAGCAGGGCTGCTTGGGGAGATGGGGAGGAGGCAGGCCGAGCTCTGGGGCTGTGGTCAGGGCG
AGTCAGCTGGTAAATGTTGCCGCAGCAGAGGGAGGGGGCTGGCCTCATGGAGGGTGGGTGGGGAGGCAAG
CAGTGGGGAGGCTGAGAGCTTAGAGGGGAGTATGGAGCAGAGGGTGGGGGGCAGAGGGAGCAGGACAGGG
GACCAAGAACAGTCCAAGGGCCACCTCAGATCCCCAAATATGAATCAGAGGGGAGTCCACAGCCCCAGGC
CAGGCCCCTGTTCTCAGTTCTGTCCCCTTTCCTCTGGCCTCAGAAACACAATGGCTAAGATGCATTGAAC
GCCGACTGCCGGGTGCTATGTGCATGCATTATCTTATTCCATTCTTTTTTTGTTTGAGACGGAGTCTTGC
TCTGTTGCCCAGGCTGGAGTGCAGTGGCGTGATCTTGTTTCACTGCAAGCTCTGCCTCCCGGGTTCACGC
CATTCTCCTGCCTCAGCCTCCTGAGTAGCTGGGACTACAGGCGCCCGCCATGCCCAGCTAATTTTTTGTA
TTTTTTTTTTTAGTAGAGACGGGGTTTCACCATGTTAGCCAGCATGGTCTCGATCTCCTGACCTCATGAT
CCACCCGCCTCGGCCTCTCAAAGTGCTGGGATTACAGGCGTGAGCCACCACGCCGGGCCTATCTTATTTA
ATTCTTACAATTGCCGCATAAGGTAGACACTATTATTTTCATCATTCTATCTTATTAAGGGCTAAACTGA
AGCTCAGAGAGGTTAAGTCACTTGCCTAGAGTCACACAGCTTGTTAAGTGCTAGTGCTGAGGCTGGAATG
AGGCTGGCTTCTGCGCTCCTTAGGCTGTCTCCTGCGTGAAGGGGGATGGAGTGAACAGTGCCCCCCGGTG
GTGATGTGATGAGAATTCGTAGTCCTCCCCTGGACTTTTGCTGCAGGAGAAAGCCTTTCACTGCATAATG
GCGCTCTTGCCCTTGGGGAGAAGTTGGGGGCACTTCATCTCCAGCAAGAACTGCACACACCACCTGCGTG
TACAGGCACACACACCAGATGGAGGCACTGCTGCAGACATGCAGCTTCTTACCATTTCTTCCTGCCTCCT
GCCCTCCTTCCTTCCCTGCCTCTCCCTCCCTGGACATACAGCTCCTCTTTTCTTCTCTTCCTCCCTCCCT
TTCTTCAGACATAGGTACCTTTCCAGGTAGTTAGCATGTAGCAGGGCTCCGCAAGGCTGTTGGATGAACT
GAGTGACCGTGCTTGGAGGGCCTAGTAGAGGGGTAGGAAGACATCGAGGTGGGTAAGCCATGCCTGCCCC
CCAGGGGTGGGTGAGGCCTGCTGGGCAGCTCCTCTGATCAAGCCTTCTCCTGCCTGTAGTAGAAGCTGCA
GCTGGGGTGCATCTTTGTGCTGGGGAAACACAGCACTTATGTGTTTGCACATGTGCGTGCGTGTGTGTGT
GTGTGTGTGTGTGTGTGTGTGTATGCATCTGTGGGCAGCATTGGGGCTGCGGTTCCTCCTTTCCCCTCCC
CATCAAAGTCTGGCCCAGAGGCCTGGCAGCCACTGATTTCTCGTGGGCTCTCTCCACTCTGGTCTGCAAG
TACAAGGGCCTCCATCCTTGGGTGGGGTCCCTGCCAGTTCCAGTGGCAGTCATTCTTGCTGCCCGCGGTC
TGTCACCTGCTCCTGGGCATGTTCTGCCTTTCTTGCACTCAGATGCCCCCACATGGTCACTTAGGGCTGG
CTCTTTGCTTGACAGTGGTTGCGGTAAGGTCATTTCAAAGAGCATCCTTCTTAGTATGTTTAAATAGCTA
TAGAAAAGCCGCTGCACCCTCACCTCTTCAGAGACCCAGCCAGCGATAATGGCGCGCTCACTCTGATCTC
GGGGGAGTTAGGAGAGGGTTGGGCGTGCTTAGCAGGTTTGTGATGTAAACAGTGCCCATCCCTGAGCTGA
GCAGGACCTACATTTTGGGCTTCGGTTTCTCTAAAATTCTCTTAAATTGGACATTATTACATGTCAAATG
TAGTTAATCTTGGTCAGTACAGTCTTGGATGGGGGTTGTTTGGCCCCTGCAGAAGGGCCTGTGTTGAAGC
TGGACTCTGTCACGTGACTTGGAAACATGGCTGTGAGCAGGAAAACAGCTCCTAGTTGGCTTGAGAGGTT
CAGACGAGATGGTGTGTGCAAGTGGTTAACACCAGGACATAGCATATGTTAAGTTAGCTTTATAATTTAT
TTTGGATATCTACCTACCCATCCACCCACCCACCTACCTGCCTACCTACCTACCTACCTACACCTTTGTG
GTTTACACCCACTGCCGCCCCATGAATTGCAGGGGCGTGGGAGAAGAGTAGGAAACCATGGGTGGGGACA
GAGTGTGGGCTTAGAGTCCACTGAGAGCCTAGCACGTGAAGGGAATGTTGTCTCTCTTTGGGTCATCAGT
CACTAAAGGCCTGAGTTCCCTTTATCCCTTGAACAAAAGTAAAACAAGGCAAGTGGTGATGAGACCTAAC
TCCTGATGGCAGGGGCTGTAGATCCCTTCAGGGGCCTAGGTGAGGGGCCACAGTCACTTGGGTCCCTCCT
GCTCTTAATGGCCGGGTTAGGGATGGAGCCCAGCCCTGCTGATAAGGTGAGTCGCCCAGGGCCAGGGCAG
GAAAGGCCAGGCTCAGCTGTGCAGAATAGAAGAATGGGCCAGGCGCAGTGGCTCACACCTGAATCCCAGC
ACTGTGGGAGGCCAAGGCAGATGGATCACTTGAGGTCAGGAGTTGGAGACCAGCCTGGCCAACATGGCAA
AACCCCGTCTCTACTAAAAATACAAAAATTAGCCCAGCGTGGTGGTGCGTGCCTGTAATCACAGCTACTT
GGGAAGCTGAGGTGGGAGAATTACTTGAACCTGGTAGGTGGAGGTTGCAGTGAGCCAAGATCTCGCCATT
GCACTCCAGCCTGGGTGGCAAGAGCAAAACTCCATCTCAAAAAAAAAAAAAAAAAAAAAAAGCTGCGGTG
TGTGGTGGCTCATGCCTGTAATCCTAGCCCTTCGGGAGGCTGAGGTGGGCAGGTCACCTGAGGTCAGGAG
TTCAAGGCCAACATGGTGAAACCCTGGCTGGGAAAAAAAAAAGAAAAAGAATAGAGCTGAGTGGTGGAGC
AGTGTCCCAGGATAATTGGGAAATGAGCCTTTCCTCTCTGGTACCTGGGCTTGGCACAGGAGTCCTGGTC
ATACCCTGCAGCTGCCTCTGCAGAGAGCCACAGGGCATTAGGGATATTCCTTGTAATCATCCCACGTAAT
GTTTGTGCTGCATGGAAGGGCATGTAAAGGAGTGTTCTCTGCACGGTAATTGACTCATTCTGTCATCATG
ATTAGTTAGGGGGCGCCCAGAGCAGGATGATGGTCAGAGGTTCCTCATCCCCATTTTACCGATGAGGAAC
TGGGGCTTAGAGAGGCCACACGGCTACAAAGTAGTTTTATCTGGATCTTCCCCTCTATTAAGCAAGGGCT
GGCAGAGCTGGGTGTGCTACTGAAGGAGCAGACATGTGTGGGCTTGGTGCCATGGAAGTGGCCACAGGCA
GAGCAGTAGCACACTCCAGTCTGGGCTCAGCATGGCATCAGGTACTTGTTTACATGTTGTTGTCTCCCTC
TGAACCGAGTTTCCTATTTGTTTATCTTTGTATCCAGTACACAGTAGGTGCTCAATAGATTTTTTTAGTG
CACCCCTGTGTCTGGCCTTGTGACATAGAGACCTCAGAGGTGGACAGGGCTGAGGTGTGCTGCTCGTGGG
TGTGAGGTCTGGCCCACCCTGCACCTCCAGAAGACAATGAGGCCCCAGCCAGGTGGACAGGCTGTGGACA
AGGGGCTGCAGCCCTGGCACGAGGGCCTGGGAACCAGATGGGCACCACCGTTGTCAGCTATTTAGCATTT
CTGGAACACCTCTGTGTGCAAGTACCCAGGAGCTGTGGGGGACTCAGAGGCGCCAATGAGACACGGCCCC
TCCCCTCCAGGAGAGTGGCCTTTCAGGAGGATGGAAGGCTTTCCTCGGTAATTATGACACGACCTAAGCA
GTCAATGGTTACTTGTCGGATATTGTTGAATAATTGAAATAAAGGGCTGATGAGCCTTGGGAGGTGACTG
AGATCAGAAAAAGTAGGGGGCTTTGGAGGGCTCTGGGCCCATGCGCATCTACAGTGATTCCCAGCCCACT
GCCTGACCCCTCACGCTGGGATCAGCCTCTGCAGGTTGGCCTGGGCACGGGTGTGTGTGCAAGCATATGT
GAGTGTCTGTGTGGCATTGGGGTACGCAAGCGTGTGGCTGTAGCTCTGCAGAGCCTGGTGCTCAGGCAAC
CCAGGACAGGGCTCTGCCTCTTGCAGAAGCCAATTCGTTTCACTGAATTCCATGACCACAGACAGTCCCT
GGGAGGCCAGCCAGCCAGCTGGCAAAGGCTGGGCATGCTTCTTGTCCCAGGGGGCCCGGGCAAGTAGCTG
GCTAGATCCGGCAAGTTCATCCCCTCGACTCAAGCACCAACTCGAGACATCTGCTCAACCGATGCTGGGT
CAGTAGCATCCGGTTTAAGTACTGAAGGGAATACACGTATGTCAGGCTGGCTGTCTCTAGGTGAGGGCCT
CAAGAGACATAAATCTCTTGTGGGGAGGGGTCCTGTGTCTGGGCACAGCCAGCCCAGAGTGTCTGTGCCT
GTGAATGCCAGTGTGCCTTGACCTTGGATTGCCTGTATCTTTATGGTGTCACTGTGTGTGTGCCTGTTGA
CATGGTAGGGTATAGTGGCATGTTGCAGTAGAAAGACCGATGGACTGGTAAACAAAAGATGTAAATTTTG
GTTCTAAAGGCCTCCTTTCCTGGCTCTCTGATCCCTGCAAGTCAGATAATCTCTCTGAGCTTCAGGTGTT
TCATCTGTGTAACAGAAAAAAATAATACCAGCTCTGCTTTCTGTAAAGTGTAAGAATACAATGAAATAAC
ATATGTGAAGATGCTTTGTTAATTACCACGTTCAGTGCAAATGTGAGAGATTATTATCGTCATAGCCCAG
CGCATCAGAATGAGACGTTGTATATGGCTTAAAATAATCCATCCAGTGAGCAGTGTTGAAATGTAGGTCA
GATCGTAGCCTCAGATGTTTAGAGAGAGGAACCACCTAATCTCACAGGCCACCAAATGTGAAGGTGCTTT
GAAAGCTGCAAAGAATGACATAAATGCAAAGTGACAATTATGTTTAAGTCTAGAAGGGACGTTTTAGTCC
TCTTGTTTCCTAATCTGGAATCGTAAAGATCCGAGAGGCACTGGAACCTCGTACACAGTTGTATTTGTAC
GGGTTTGTAGATTTGTACGTGTGTCTCTGTTTGCGTCTATGGACTCACGTCAGGCACCTACTCTGTGACA
GTCATTCATCAAGTGCCTGCCTTGGACTAAATGCATCCGTCTAGGCATTCTGGATGCCAGCATGAATCAG
ACACCCACACTTCCCTCAAGGAGCTGACCGTTGCCTTGCTGGGTAGGGGAACTATAGGAGAGGAAGGGTC
TTTCAGCTCAGTCTAGGCAGGGGATGGGAGGAGGGGGCCTAGGGGCAGGGAATCCAGGATAGGAGGCTGG
AAAAGCGAGTAACAGTTTGCTGATAAACCTGAGGGGAAGGTGGTGTGGTCAGAAGAACAGTCTATGCCTA
GGGGCTGAGGCCGGAAGCAGTTTGCTTTCGCGCTTGGTGCTGTCTGTTGGGACCACAGTGTGGAGAAGAG
ACCAAGGCGATCCAGCCAAATCCTGGGGACCGCCCTGCACATGAGCTGAGGAGCTTGGCTGGATCCTCCT
GGCCCCCGGGAACCCCCGTGGCAAAGGTTTAAGCTCTGCAGAGTGGCATAATCAGCTTTGCATTTTGGAA
AGCTCCTAGAGCCCCAGTATTGCCACATATCTTGCGGCAAGAGCGCCCGCCAGATGTTGGGCTCTGTGTG
ATAGTGAGGGATCGCTGCATATGGTGGGCGGCGTGCTGGGATTTGCACGAGGCAGGGAGAGGGGGATGGG
GAGGATGGAGTGGGAGTGAAGTACGAATCAGGATGTGTGGAGCGGGGGAAGGTAGCAGTTCTCGAGTGAC
TATCACGTGTCAGCTTTTGCTACATCACCTCATTTAATTCTTATAACATCACTGTGAAGTAGATATCATC
CCATTTTATAGATGAGGAAAGCAAGACTCAGGGAGGTTAAAGAGCTTGAGGTTACTCCTAAGCAAAGAGC
AGAGCGAAACTCCCAGCCCGGCTCTGCCCTCTCCAAAGCCTACGTGTCTGGAATTTCCACCCCCTCCCCT
CTGCGCTGGAAGTGTGGACGAATTCAGTCCCCTGGCTTGACAGATGAGGAAACCAACCTGGGCTCAAATC
TGTTCCCGCGAACTGTGTTACTGTTCTTCAGCTGTCCCATGTCACCACGGATCAGATTCTTTTCTTTCAG
AGTATTAGCAACGCCTCCTAATTCAGGGCTACTTGCAGATTTGATAAGCATGTTTTACATTCCTTTGCTC
AACCCATCAGCAAAGATATGGAATAAAACAGACCCCGTGACAGATGCGAGAATGAACCACTTGCTACGCT
TCCTTGGAGCCATTGAGGGGCACGGCCCAAGCCATCTGCTCACCCTTCTTGGCGTGTTAGCCCCCTCCCT
CCGCAGAGGGAACCTCAGGCAGGCCCAGAGCAGAAGCATCGCTTCCCGCTTTGCCCTAAGACTCCATCGC
GGAAGAGTTGGCAGAAGAGAACATGCTTTTCACAACGCTGCTCTTAGGGCCAAATGTCAAGGGGAAGGGC
CAGGAGTTGTTCAGACCTGGCTCTCGTGGAGACTCCAAGCACGACCCAGAAGCATGCGGGGCCTGAATGC
TGGGCCAGCCGTAAAGCTCCTTGCACCAGGTGTGGGGTACACACAGCACCCCTGCCTGGCACTCCCCGCC
CTCTCTGGACCTGTCAGGACCTGGAGAGGTGGCCTGGCACCGGGGGGTCCACCCCTTCCTCCTGGGCTGG
GTTGCACGGAATTGAATCCAACAGGGAAAAAAATGTGCCGAGCACCTACAGGCCCTGCACACGTGGTGTA
GGTGAGAGATGCCAGCAGGACTGCATATGGCAGAGGGGTGGCCTGTCGGGGCCCCCAAAGGCTTTCTCCG
CATGCCCAGTGGAAGCTGGTGCCCATCGTGAAATGTCAAGCGTCTTTATTTTTAGGTCATTGCTGTGTGG
TAACTGTGGTTCCCTTTCACTGATCAGACGCTCTGGTTGGCTTTCACATCTCTGATTAGTTCAGAATAAA
AGAAAGTCAAATGACTCCCTCTGTCACAAATGCAGCTTGTTAAAGAAATAGTTTAAAGCCCAGAATTTAT
GGGCAGTAGGAGTTTTTCCAGAATCTGCCCTAGGCGCTGGGGATTCAGCAGTAAACAAAACAGACAAAGG
GGCAGACCCTTTGTGAAGCTGTCTTCTCAAAACCTGAGAAGCAGACAGGATGGACACCTCCCTACCCCCA
TTTTATGGATGAGGAAACTGAGGCTCCGAGAGGCTGCTCCTGTAACTAGGAAATGGACAAGCTGGGCTCA
TGCCCACTTTGCTTTTTCCATGACCTCACATGACCCCTAATGATTTTACAAGTGCTGATCAAGACGGTGC
CCATTTGGCACTGGTTGAATCGGATTAGAAGCCCCACCTCAGCCTGAAATTCTTAGCAGTTGTGTAGATC
CAATCAGAACCTTCACAAATACTAAGCTGCACTGGCAATACGTCCTCTCTGTGTGATCCTTTCTTACACT
CGGGGGTCTGGGGTCTTCAGAAGATGGTTCAGAAGCAGCAGGCCTCCCCTCCCATGGGGCGTGTGCCCTT
GGGCACCCTGAGCCCTCATGAAGGCTCCCGCGAGTTTGCTCCTCCTCCCACAGCCTTTCTGCCCTCACCG
CCCGTCTGCTGTGCTTTCCTGAAAAGAGCCCCTGAAGGATCTCCGGCCTCGGGGCTGGCCTCTCCCTCCA
CAGACGGCACGGGGCTCCCCAGTCCCCTGGCATCCTTTCCAGATGCTTCCTCTGCTGTGCTCCTTAAATT
GCCCTCTTTGGTCCTTCTCCAGCCCTCACTGCCGTGTCACTCTGCACAGCTTAGTGACAGTGCTCTTGAA
GGTCAGCCATTCTGACCCCTAGCCAAGCCTCCTCATCCTCAGTATCTTTCTCAGACAGCTTTGGTCTCCA
GTGTTGGCAGCCCTCCCCTTCCTTGCTCCCACAACACACCAGGGTCCTCTTCTGCACCTCCGAGCATTCT
CTCTCTATCTCCTGTGTGAGCTCTGCCTCTCTCCAGCTTGACTGTGGCCACCACCTATGCTAAGACCGGG
CACCTCTGTTCACCCTCCATGTGCATGTCTTTGGAGGCCTCCCAGAACTTGAGCCATGATGGTCTGCCCT
GCAGACATGCCTCAGCTGGAGGATGGATGGAGGGGCCAGGGATGCTCTTCCCGCTGGTCTTATGTCTACA
GCTGGTCACAGTAGGTTTCCATTTGTGTGTCCCACTGTCACCTAAAAATTAATGTGGCAAACCACAAGGT
ATTTTGCAATCACAGAAAATGAAAGTTGAGTAGGTTGTTGAGGGCCATCTTACTCAGGTCTCCAGGAGAG
CTGTGGACCCTCTTCTACCTCTGTGCTGCCAGCACTGCCCTGGGTTGGCCCCTCCTTGGGGAGGGCCCAA
AAGCCATCTTTGGCTCCTGACATCGTGCAGGGTATGAAGAGCAGCCTGTGGCCCATCTGCAGGGTCAGGA
CGTCTTTCTCCCCACTTCCCCAGCATCATTTCTGCTGGGGATTAGTCATGCCAGAGCCCAGCTGAGTCAG
AAGGGACTTGTGGGTGGTTTGTGGCCAGGCTGGAGTAGTGAGGCAGGACTTTCTAGGTTTATGTCTCTGT
GCCCCAGGAGACAAGCATGTCAGGGCCCAGCCGTGCACAGGTGTTATGTGAGGGGGGGGGAGTAAAACAA
ACTTCTGTTCCAAACTCGGTGAATGCAAGAGATGGAGAGGAGCTGAGATTCGGTCCCTGAGATCCTCCTG
CAGGCTGACAGGCATGCGCGGTGCCAGCTCCACATGGCAGGGGTGTTCACAGCTGTACAAGTCCTCCACT
TTCCATAGTTGTCCATTTCGAGTATTGCCTGGACTTCAAATAGATATTGCTAAATTTAATATTTCTTTTC
ACTTGATTTTCTTTTTTTTTTTTTGAGACAGTCTCGCTCTGTCTCCCAGGCTAGAGTGCAGTGGCGTGAT
CTTGGCTCACTGCAACCTCTGCCTCCCAGGTTCAAGCAATTCTCCTGCCTCAGCTTCCCTAGTAGCTGAG
ACTACAGGCGCGTGCCACCATGCCCGGCTAATTTTTTGTATTTTTAGTAGAGATGGGGTTTCATCGTGTT
AGCAGGATGGTCTCAATCTCCTGACCTCATGATCCACCTGCCTCAGCCTCCCAAAGTGCTAGGATCATAG
GCGTGAGCCACCATGCCCAGCGATTTTATTTTCAATAAAGGTGATTCATTAAGTAGATCACAAATGTAAT
TGTGTATCTGTGAACAAAAGCTTTCTCATATAAATAAACTTTTAACTCAAATCCTTTATCAGAGCTTGTG
TTATGATTTTATGGTTCGGAAAATGTGGTCATAAAGAGAGAGAGATGCCTTCCGGGGGGACAGCAGCGTC
TGCCCTGAAGTTCCCCAAAGGATGTATTATTTATATATATATATATATATATATATATATATATATATAT
ATATATATATATATATATTATATTATTTAGATATATAAATATATATCTAAATATATATCTAAATAATATA
TTATTTAGATATCTAAATAATATATTATTTAGATATCTAAATAATATATTATTTAGATATATATATGGGT
GTGGGTATTTTTATAGAGATGGAGTCTCACTATGTTGCCCAGGCTGGTCTCAAACTCCTGGCCTCAAGGG
ATCCTCCTGCCTTGGCTTCTCAAAGTGCTGGGATTACAGGCATGAGCCACGGTGCCTGGCCAAGGATATA
TTTTAAGTGGGGCTCTGAGGGGTGCCTCAAGCCTCATCAGGCAGGAGGGGAGTTGGTGGGGGCATATTAC
ACAGCCCTGGAAGTGTGGCTCGTGGGACAGGAGGAGGCTGGGGCTGGAAGGGAAGCCAGGGCCAGCCCCA
GACAGGCCTGGAATGCTACACTGCTGGGATCCACCTCTGGCCTCACAGGCTGTGTCTGTGCCTGCAGGAC
GGAGAGGAGCAGGAGGAGCCGCGTGGCAAGGAGGAGCGCCAAGAGCCCAGCACCACGGCACGGAAGGTGG
GGCGGCCTGGGAGGAAGCGCAAGCACCCCCCGGTGAGTGTCACATCCTACACACCCTCCTGTGCACACAC
ACAACACACACGTGCCTGGCCTGTCGATGGCAGTGATGTATGTCCTGCAGGGACCTGGAGACATTTAAGA
CCAGGTGATGGGGTATACCCCTCCAGCTGCAGGTACCCCATTCTTCAAATCCCACCCTCACATTTCCCTG
TTTTCTTATTTATTTATTTACTTTTTTACTGAGACGGAGTCTCACTCTGTTGCCCAGGCTGGAGTGCAGT
GGCGTGATCTCGGCTTACTGCAACCTCCACCTCCTGGGTTCAAACAGTTCTCCCACCTCAGCCTCCCAAG
TAGCTGTGATTACAGGCACGCGCCACCATTCCCGGCTAATTTTTGTATTTTTAGTAGAGGCGAGATTTCG
CCATGTTGGCCAGGCTGGTGTCGAACTCCTGACCTCAAGTGATCCGCCCACTTCAGCCTCCCAAAGTGCT
GGGATTACAGGCATGAGCCACCGCGCCCGGCCACATTTCCCTGTTTTCTGCATAGAGAAGTGGAGGCCCT
GAGAAGCCAGGGGACTTGGCTGGTTGGTCTTGGGCCTAAGACTTGGACCAGGCCCCTGACTCCTGGCCTC
CCTCTGGAAGGGAAGCAAGAGGGCCTGCCAGAATCCCAGGCTGGACTAAGATCTCCCTGGACTATCTATG
AAGGCCCCTCCCTACCACACTCTCACCCAGGGTCTCAGCCCCCACCTGGAGGAAGGGAACTGAGGCCTGG
GAGTCTGATCAAATGGGAAGGGGGAGATGCAGGCTGAGGAGTCTCTGTGAGGCAGCAGGAGAACCAGGGC
CCTGGGGAAGGAAGTCCTGCAGGCATGTAGGCTGCAGGGCCAGCCCCAGCGCCAGGCTTCTGGGAACCAG
TGCCGCTGAGGCCTGGGGGCAGCTCTGGAGAACAAGACCCCTGGGCTGTAGGAGCCTAACTGGTTTATGT
CTTAAACTCAGATGTGGAGCAGTCATTATTTCCCAGCACCAGAGACAGCTGGTTCCTTTCTTTTACCCCC
CTTCCAGCTGAGAATGGTAAACTTGGAGCAAGAAGGTAAGGGGGAGTCAGAGGCTCTGGATTTGTTACTC
AGCCCTTCAAAGAAATGTTCTCAGCAGGCATGGAGCCCAGGACTTGCTCCCTTTGGGTAGAGAGCCGGGT
TGAAGGTGACTGAAGTGAAATGGGACAGTAGAGGCGGGGGGGGTGGTGAGTTCCTGGAGGTGGGGGGTGT
GGGAACCTGCTGTGTACTGAGATGCACCCCTGCCAGTTCTGCCTGAAGATTTGAGGCGGGGGGCAGGGGG
GCGGAGTGAAGTCATTTTACTGGTAAGTAATTTTAAACCTTTTAATATTAAAGCAAACGTGGATATGTAA
TGAATGAAATTCATTCTGGAATGAAAAATTCACGTGATGTTGAAAAATAACACGGGGCTTCAGAGAGGAC
TTTCTGGCTGGCAGCAGACTCCAGATTCCCAGGGCCCCTGCACCCTCCTCTGCCCACAGGGCACCTTAAT
GGAGAAGGTGTGGGAGGAGAGCCAGGCCGGAGTCAGAGCACACTGGTGACTCCACATTTGCAGCGTGCCC
TGCCTCTCTCCTGAGGCTTGGCAACGTGCAATATGCTAAGCAAACTCCCCCTGTCCCCGTCCAGTTTCTG
AGGACAAGAGCCACCACCTGTAGCAAATAAAGACCCAGCAACCCTTTGACTCATCTTTGTGAGTCTCTGG
AATCAGAGGGTAGCCACATCGCTGAGAGGTGGAGTGAAGCACTCGGGTGAAAAGGTACAAGGAAGTCAGG
GACAGGAGTGTGGGGACATCACCTAGACAATGACAGAGAAGAGGGGCACAGCCGAGTGAGGGGAGAGGGG
CCGGCAGTCCTACATCCCCTGGCCTGAAGCACGCTCCAGGGCAGAAGGAAAAACACTGTCTTTGGGGTCC
AAGAGACCTGAGTTCAAATTCTGGCTCCACCACTGACCACCTGTGTAACCTTGAACTGCTGCTGCCTGAA
CCTCAGGTTCCCCTTCTAAAAATAGAGGAGAAAAGGATGCATTTCTCCTTGCCCCTGTGAGAACGAAATG
GTGCAAGCACCAAGGAGCCTCAGCAAAGGTCGGGCCTGCCCCCGCCTGGCCAAACCTTTCCTCTTCAGGA
GGCCACGGCAACCGTAGTTTGACAGAAGAGCAGCACCTTGATTTAATGCTTCCCAGCATGTGTCCTTGAG
CAAGTCACCTAACCTCTCTGGGCTGCTTCCTCATTGGGAAAATATGGCTGCCAGTAAAACCTGCCCTGTC
CACCTCCTGGGGCACTTGGCAAACAGCAAAAGAGTCCAAATGTGCAGGCTGGGCCAGGCGCAGTGGCTCA
TGCCTGTAATCCCAGCAATTTAGGAAGCCAAGGTGGGCGGATCACCTGAGGTCAGGAGTTTGAGACCAGC
CTGGCCAACATGGTGAAACCTTGTCTCTACAAAAATACAAAAATTAGCCGGGCATGATGGCGGGTGCCTG
TAATCCCAGTTACTCGGGAGGCTGAGGCAAGAGAATCGCTTGAACCCGGAAGGGGAAGGTTGCAGTGAGC
CAAGATTGTGCCACTGCACTCCAGCCTGGGCAACAGAGCGAGACTCTGTCTCAAAAAAAAAAAAAAAAAA
AAACAATGCAGAGCTGGCTGTGTAAAAAACCTGTTCCACTGCAGGGCCCAGTGTCCACCAGGCTGGGGTG
CAGGCCTATGGGGTGGGGGCCCAGCATCAGCCTCTCAGCAGCCCTGGGAGGCGGGGCGCATCCCGTGCCC
CTCGTGGTCTGGATGTGTTCTAGCCCAAGTCCTAGGTTACACCTGCCGTCGCCTGGCCTCTCAGGAGAGG
CCCAGGGTGAGGAGGAGCATGGTAAAGGTGAAGCTGATTGGGAAGTCAGCTGTTGGGAAAGCAACTCCTT
GCACATTGGAGGAACCGAGAAAGACTGACCCCGAGGACAGCAGCCAGCATGGCCCTTCCTGGGAGCCCAT
GTTGGGGGATTCCTGCTGCAGCCAAGGCTCAGCCCTTGTGGTCGCAGGTGCTGGTCCTGGCCTCTTCCCC
TCCCATGCAGGAGCACAGGAGAGATGGCTTCTGAGGACCTGTTGCAGCTGTGGCCCTGGGAATAGATTTG
CCAGGGAGCTTTAAAGCAGCTGAGTGTGTCATCCAGCTAAGCCTGGGGAAGGAGCTTGGCTCAGGTCCTG
ACAGGTGTGACAGGGATGGGGACTGGGAAGTAAGAGATGAAACCCTGGCTGGAGGCTGTGAGCTTCCACA
GCCAGCGCTGGACAGGGAGGGTCCAGATATACCCACTAGTGCCCTCACCAGGTCCCACCCCCAAGCCCAA
GAAATCCTGGGCTCTCTCCTCCTTTTGCCCCCAAGGACTGTCTGTCTTGGCCTTCAACAGGCATCTGTCC
CTGGGTGCCTTCTGCCCCCTGGCATTGCCCTCACCCAAACCAGACTCCACCTGCCCCTTGCATGCCCAGG
CCAGTGTGCTCCCTTCACGACAAACACTTTCCACTTCCTGAGCACTGACCATGTGCGGGAAGCCACCAGA
GGAAGATGCCTTCTATTTCAGTCAAGTGTGCTGGAGAGAGGAGGCCACACATAATCACAGTAAATGTAGC
ACCGGCACGTCCTTCTTCAGGAACCCCCCGGGACTCCATTTTTTAGGGGACAAGATCTGAATTCCAGCCC
CTCACCATCTGACCACTCCTTTCCCTGCTATGCCTGATGCCACCCCTTTGGCCACAGAAACCTCACCTTC
TCACTGCTCCCCAAACACACTAGCCAGCCATTCCTCCTGCCCGCCCCTTACACAGTTCCACAGCCTGCCT
TCTCTCCGCCTGCCCCAGCACCACAGCCCGGTAGAACCTAGCCTGAAAATGAAGCCCAGTTCAGCCACTG
TGACCCACACTGGCCTTTCCCTTCTCCAACTGTCTCCAAAGTTAATAAAAATGAACTGATAAAGACAAGT
TCCAAGCCCACCGATCACACACATGGGACTGCAGTGGTCTGCAGACCTCACTTTGAGAAGCATTTGTTGT
CACCTGTCGGTACAGAACTGAGTATAACTATTCAGAAGCTTTTATGTGTGTGTGACAATGTTGCCACATC
CACGTGCTGTGATTTTATATTTTACAACCGTATCATTTCTTGGTGCACTGAGAATTTAAAAATCTGGAAC
CAAAGCTTGAGCTTTCATCCCTGACAGTTTGAAAATGCACCGCCATTGCATAGTCGCTGAGCTGTGCCAC
ACTGAGTTTGCCTGCCTCACTGAGGACAGAGACCAGCAGTAAAGGCTCATGGTTATGCAGCATGTTACTG
CCTACAAAGGCGTTTTACAGGGGTTACCTTATTTGATGCTGACAAACCTGCGTGAGGTAGTTGGGGACCG
CTTTCATTATCCCCACATTCAGAAGAAGAGACTGAGGTTCAGAGAAGATGATCAGGACTTGGACTTCACA
CCTCCCAACTGCAAAGCCTATGGTGCTCTTTCCTCCTTATCACTGCATCCGTGCAGTTTGGCTACTGGGT
ACCTACCCAGGGGTTCTCTGTCAGTTAATATGTGAATTCAGGGCTGCTGGAAAAAGTGTGTGGAACTGGT
CCAAAAAGCATACAAAGAAAATGGAACTGAAGAGAGTTATTGACACAAATACCAGGGAGCCTGTGTGTAA
GTGCCGAAGGCTGCAGGGGGCTGAGGGCAAAGAGGTGCGCCTGCTTAAAATCGGCAACGGCCTAGTTGTC
TGCAGCACTGGCCAGAGAGCCAGGATGCTGCAGTCAGAGCCGGCAACAGGAAACAGGAAGTCAGCTGCAG
GGCAGGGCGGGGCTTAGGAAGGGAGCTGGCACCTGGGCCAGGGCCTGCGGGCTGGCCAGTGCCCATGATA
GGGAGGCGGGGGCTGGTGCGTGGAGCTGAGCCCGTGTGTGGGTGTGAGGTGGGGAGGAAGGGGCAGCCTC
TAAGGATGTTGCAGGGTGACAAGTGTGGGTGTCTGAGGCTGGTTCCTGCCTGGTTGCCAGCCCAGACAGT
CCTGCAGAGGCCTTTGTCCTTCATGGGTGGTTCCGGTTGGAGCCCTGAAGGGAGAGTTTCTGCCTTTGCC
CACAAGGCCCTGGGGGGTGAGTGGCCCTGTGGGGCTCCATCCCCTATTTACTAGCGATGCGTATTTATTC
ATTGACGATTTATACCCACCAACTTCCTCCAGGGTTATTTTTGCTCAGAGGGAGCACTCTGAGGTCACCT
CCTGCTAAGAATAACCCAGAATGGGAGGGAGGAGGAGGAGGGGGCTGGGCGCTGGTGCTGAGCTCAGCTG
AGCCCAAGCTGGTGTTAGGGCTGACCCTCCAACACCGCAGGAGTGAGCCAGTCCAAGAGGGCAAACTGCC
CCACTTCAGAGCAGATGACTTGGGGTAACCTAAATGGGGAAGTCAGGGAACCATTCTGCTGCAGGCAAAA
GTCAGCTAGGTACAGCTTATGGCAGCTCTTGGAGGTTTGCAGCACTGGCTAGCTTTCCAGCCTGATAGGA
CCTTTGATTCTTCTCCAAGTTAAGACAGAGCTCAGCAGCCCTGCTTGTTTTCAGATCAGAGCCGCTCCTG
GGCTAACCCTCCGGACCTCGGCTGGGTGATATGGTGCCCCTACCTTTTATGCATATAACTTCCTGACATC
GTGTCTCCCACTCCTGGGCTGCAGCCCTTGGCCCACCCCTAGAGGTGGCTTTTCTTCCTGCGAGCCTGCA
TCAGTACACGCTGCTGCCCACTCTGTCTCCTCATGTTTTGGAAGGCATGAGGAAGACAGGCCGACACACT
GCTTGTGAGGTAGAGCCTATGGTTATGAACCTTGCAGCTCCACCCACCCACTCCCCGACCTGAGCGGTGA
AATCAACGCCCGCTACTGGAGTGCTGCATGGAAGGGCCTAGCCCATGGTGGGGACTTAAGTGCAGTGGGA
CTCCTAAGGATGCCGGGGAAGCCAGGGAAGATGGAGGAGGTGGGGCTTGACCTGGGCCTTAGAAGGGAGG
TGACATTTGGAGAAGTCAAGGGGAAAGTGAGAGGCACCCCCGACCCCAGCTGAGCTGGGTGTGGAGGAAG
GAAGGCATGCAGGGCAGGGTGAGTGTGGGGAGGCCCAAAGGCAGGGGCCATGAGCACACAGGCACAGGCC
TGGCTGAGGGGACTAGGGATTCATTGAAAAATGTATACGGGGCTGGGTGCAATGGCTCACACCTGTAATC
CCACCACTTTGAGAGGCTGAGGCAGGTGGATCACTTGAGGCCAGGAATTCGAGACCAGCCTGGCCAACAT
GGCAAAACCCCATCTCTACTAAAAATAGAAAAAAGCCGAATGTGGTGGCACATACCTGTGGTCCCAGCTG
CCCAGGAGGCTGAGGCACGAGAATCGCTTGAACCTGGGCAGGCAGAGGTTGCAGTGAGCCGAGATTGTGC
CACTGCACTCCAGCCTGGGCTACAGAGGGAGACTCTGTCTCAAAAAAATAAATTTTTATATATATATATA
ATAAATAGTAGCTGGGTATGGTGGTACACCTGTGGTCGCAGCTACTTGGGAGGCTAAGGCAGGAGGATCT
CTTGACCCCGGGAGGTTGAGGCTGCAGTGAGCCATAATTGTGTCACTGCACTCCAGCTGGGTGACAGAGC
AAAACCCTGTCTCAAAGAAAGAAGAAAAATCTTCTCAATCTTGATTGGAAAGGTAGTTTGCAGGCAGCCT
GAGGGGCTTTTACTCTTTATCCTCTCTGAAAAATGTGTTATCTGGAAGGTTGTGAGGAGGCGGATATGAC
GTGATGGGAGTCGTGTTTGAGAAATTGGCTCTGGCACCCATGAGCATGGCGTTCATCCCTGAGCATGAGC
ACATGCTGGGAGAGGGGAGGGGGCAGGGGCGCCAGCAAAGTCCCAGGGCCTGTGGGGGGTGGCAGTGGCG
GGGCAGAATTGGCCAAGCCTATGCTGCTGGCCCTGGGAACGCTGGAGAGGAAGGGGTGAAAGGAAGCCCT
AGCAATGGCACAACAGGGACAGCATCAGCGTTGTTGTCAGAGACAAGGCTTCTGGAAGGGAGCAGATGGG
GAAGGGGCCCCTGTCAAGGCCACCTTACATAGCTGGTCTTACTTGGGGAGCATTTAAGTAGAATCTCAGA
ATCTCCTTTTTTTTTTTTTCCAAAAATAATCCCTCTGTTTATTTTCATCTCTCCACATCCCAGGGCCACC
TTCAGCGAGTCATTCATTCCCACAGGTCCTGGCCTTGGTGGCCTTCCTGGGGCCCCATGAGCTTTTCTAG
GGCCCGGGCTCCACCTGCATTGTTCCTCCTGTTGCAGGTACGCGCCGGCACACCCATTGGAGGAGAGCTG
TTGACATGGGCTTTCTTTCATCATCCCCACCTAGAGAATTTTGTACATTTTGGTTCTCCTACCTCAAGAA
AGACATAAGCGAGTTGGAAAAGCTCCAGAGAAGGGCACTAAATTGATCAAGGGGAAGGCAAGGCTGCCAA
GAGAGGACAGGCTAAAGAGATTAGGACTCCCCAGTCTGCAGAGACAAAGGCAGAGGATGTGATTAAGGCT
ATGAAATCATGTCGGGTGCGGCGGGCTGAGCACAGCACAGCTGGGGGGCAGAGGGAGGACACCCATTAGA
AATTTCAGAAAGATAAATGAAGCACAGAAAAGATAGCACCATTTTCCATGGTGGGTTCCCTTCTTACCAA
ACCTGTCACCCTAGGATTGGCAGAAATTGAAAACAGAAGCAACTTCAAAAGAGATTTACTTAACGTATGG
GATCTCTCATACGTAATGGCTTTTTTTGTTTTTGTTTTTGTTTTTGTTTTTGAGACAGATTCTCACTCTG
TTGCCCAGGCCGGAGTGCAGTGGTGCGATCTCGGCTCACTGCAACCTTTGCCTCCCGGGTTCAAACAATT
CTCCTGTCTCAGTTTTCTGAGTAGCTGGGATTACAGGCGTGCACCACCATGCCTGGCTAATTTTTTTTTT
TAAGGGTCGAAGGGATTTATTGAAAATGAAAGTACACTACACAATGTGAGAGCAGGCCTGAGCATAGGGG
GCTCAAAGGCCATTTTTTTTGTATTTTTAGTAGAGATGGGGTTTCACCATGTTGGCCAGGCTGGTCTCGA
ACTCCCAACCTCAGATGATCCGCCCGCCTCGGCCTCCCAAAGTGCTGGGATTACAGGCGTGAGCCACTGA
ACCCAGCCAGCATAATGGCTTTTTAAAGGGAGCCTCCAGCTCACTCTGAAATCCTTTGAGGTTGACTTCA
AAGAGCACAACCAGGCCCTTTTCCAGGTGGACCAGTGTCCCCGCTGGAAACAGAACCCTGAGTGGGCCGG
CCACTCACCACGCATGGTCATTCTGGCATCCATTTGCCATGGCTTGTGACGGCCTAGCCAGGCCATGGAC
TGAGTGACCTCTAAGGCTTTGCAGAGCCCAGAGTTCTCTCCTCAGACACACGGGTGGAGAGATCTCTGGG
GAGAAGGGAGACAGGCAGCTCCCAGCAGATGTGGTAAGGGGAATGGTAGGACCTGGAGGGGGTGCCCAGG
CTGGGACCAGAGTTTCCAGAGTCGGCCCTCTGTGGCCTCTGGTCCCGCCATCCCTGACCCCACTGGCTGC
GCTTTTAGCCACCCCACAGGGAAAAGGGAAATTGGAGGAGTCAGGGTCCTAGCTCCATAGCGTTGGAGCT
TGCTCCATCCACCCCAGGAAGGCCGAGCAAGGGGGCATCTGGGGACCCCCTGGCCCCTCCTGGAGTGGGC
ATGGAGCAGGCATCTCTGGCTCTCCCTACCACAGGCCACTTCCAGCTGCTATCAGATCTCTGGTGAGGGC
CGCCAGGGGAAAGGGGGCTGCCGGAAGGGCGGGGGCGGGCAGTCATGGCCCCGTGCCAATGCTTCGGGGG
CAGAGCTGCCCTGCTTACGTGGACATGCAGCTGTTTCCTGAAACTAGAGTCAACCTGGAAAAATTCAGGC
TGGTTGAGGGGTGATGGGCCTGCAGAGCTGAGGAACTGGAAGGAGCGAGGAGTTGCGGTGGGAGTGGAGG
GGCTCACTGGCGTGTTTGTGTTGGCATCACGCAGCTTCCTTGCACCCCTCTGTCCTGTGGCTGGCAGTGG
GTGTCTGCAGGCGTGGGGGTGCCTCCTTCTCAAGTCTGTGCACGTGACGGTGAAGAGTCCGTGGCTGGCC
TGATGCCAGGGTTCCCGGTTGGCATGGAATGGATCTCACAGAGGCATTAAAAGCACCATGGCAGGCCCGG
CCCTCTGGGTGACCAAGGTGAGGTCGCCCCTGCATCTGCTGGGCTTTGGGGGAGAAGGCAAGAGGAGCTC
CCAGTGGGAAAGGAAGAGGCAGGGGAGGAGGGAGAATGGCATCTACCCACCCCCCACCCCCACAGGATCC
CCCTTCCCCGGTGATCGCTGCAGGTGGGCTGCAGCCTGGGGACAGGGCTTCTTGCCCCAGGTGCCAGGGC
CTCATCTCCCCTGTGCCATGACCTTCACCTCTCCTCCTCCTGGATTTTAGTTTTTCTGTGTAAATAACTA
TAGTAAAGCAACACTTAAAAACCACTTCTGGCCGGGCGTGGTGGCTCACGCCTGTAATCTCAGTACTTTG
GGAGGCCGAGGTGGGTGGATTACCTGAGGTTGGGAGTTCGAGACCAGCATGGCCAACATGGCGAAACCCC
ATCTCTACTAAAAATACAAAAATTAGCTGGATGTGGTGGCACACGCCTGTAATCCCAGCTACTGGTTGGG
ACTGAGGCAGGAGAATCGCTTGAACCCGGGAGGCAGACGTTGTAATGAGACGAGATCACACCATTGCACT
CCAGCCTGGAGAACAGAGTGAGACTGCGTCTCAAAAAAAAAAAAAAAAAATCACTTCCATAAAATTAGCT
GGATGTGGTGGCACATGCCAGTGGTCCCAGCTCCTTGGGAGGCTGAGATAGGAAAATCGTTGAATTTGAG
GCTGCAGGGAGCTATGGTCACACCCCTGCACTCCAGCCTGGGCAGCAGAGTGAGACCTTGTCTCAAAAAA
AAAATTTTTTTTAATTACTTCCATAAAAATAAAAAGTTAGGTGTGGTGGCAGGCACCTGTAGTCCCTGGT
AAAACTGAGGCGGGAAGATTGCTTGAGCCCAGGAATTCAGGGCCACAGTGAGCCGTGATCGTGCCACCGC
ACTCCAACCTGAGTGACAAAGGGATATCCTGTCTCTTAAAAAAAAGAAAGAAAAAATCACCTCCCCATAT
TGGGAGTTTCAGACATTGCCCCACCCTGGGACGCAAGGCTTTGCCTGGAGTGAAGGAGGGGCAGGAGGTC
TGGGTCTGGCCCAGACTTTGGGCAAAGGGGGAAGAGGAGGCGGCGGCAGGGCTGGGGAATTGGTTCCGGC
TGGGCAGGCCACAGCAGAGGGCTCCTCCTTGCCTTGCCTCTGTCTTTTGCTTGAATTGAGCGCTGCTCAG
GGGAGGGCCAGCAGCCTCTGCCCGGCCTTTCTTGGGCACTGCACTGCCACCTCTGCCTTCTCCAAACCCT
GGGGGCATTTGTGAGCTTGCGCTTTTCTGAGACTCAACCCAGAGCGGGAGTCGGCCTTCAAATGCCTCGC
CTGTACCTGAGCCATCCTGGAAGCTTTACTGATCAGAGGCGTGTGACCCAAGGCAGGCCTCAGAAAAGCG
AGTATCGCTCCAGCTCCCCTAGGATCCCTGTGAAAATTACATTTATTAATTTTATAAGCATATGTATGCC
CAGTCCTGTGTGGGATGCTAGAGAGCCATAGGTGGAGAGGCCTACAAAGTTAAATAAGATTGGCCGGGTG
CGGTGGCTCACGCCTGTAATCCCAGCACTTTGGGAGGCTGAGGCGGATGGATCACCTGGGATCAGGAGTT
TGAGACCAGCCTGGCCAACATGGAGAAACCCCATCTCTAAAAATACAAAAATTAGCCAGGTGTGGTGGCA
GGCACCTGTAATCCCAGCTACTTGGGAGGCTGAGGCAGGAGAGTCGCTTGAATCCGGGAGGTGGAGGTTG
CAGTGAGCCGAGATCGCGCCACTGCACTCGCCTGGGCAACAGAGCGAGACTCGGTCTCAAAAAAAAAAAA
AAAAGTTAAATAAGGCAAGGCTCACAGTCGAGGAGGGAGGCCACAGTAAACAGATAATTAGATTCAGAGG
TATGAATTGTAAGGAGATTGGGTGAGAAAGGGCTTTGTAAACAAACGTGGCATGCATTGGCTGGGACAGG
AGTGACAGCTGCGGTGGGTTCACCCCCACCCTTCAGGGCAAGGCTGGGCCAGCAGCTGTCCACACCTGCC
AACTACAGGGGGCTGAGGGGTCCAGATCCTTCCATTTAAAGGAGCATTCCTTTAAAATGCTCCTTAGAAG
ACAGAAAGTAGAAGAGTGGTGACTGGGGGAGAGGCAACTGGGAAGTTAGTGTTGAATGGAGACAGTTTCC
GTTTGGGATCGTGGAAAAGTTCTGGAGATGGATGGGGGTAAAGGTTGTATAACCATGAGAGTGTACTTAA
AGTATGCTTGAAAATGGTTAAAATGGTAAATGTTATGTATGTTTTACCCCAGGTTTTTAGAAGTTTAAAA
AAGTGTTTCTAGGCCGGGCACAGTGGCTTACGCCTGTAATCCCAGCACTTTGGGAGGCTGAGGTGGGTGG
ATCATGAAGTCAGAAGTTCAAGACCAGCCTGGCCAAGATGGTGAAATCCCATCTCTACTAAAAATACAAA
AGTTAGCCAGGCATGGTGGCAGTCAGCTGAGATAACACCACTGCACTCCAGCCTGGACAACAGAGCGAGA
CTCCGTCTCAAAAAAAAAAGTGAGCGATCTTGGCTCACTGCAAGCTCTGCCTCCTAGGTTCACACCATTC
TCCTGCTGCCTCCCCAGCAGCTGGGACTACAGGCGCCTGCTACCACGCCTGGCGAATTTTTTGTATTTTT
AGTAGATTCGGGGTTTCACCATGTTAGCCAGGATGGTCTCAATCTGCTGACCTCATGATCCACCCGCCTC
AGCCGCCCAAAGTGCTGGGATTACAGGCGTAAGCCACCATGCCTGGCCAAAAAAAGTGTTTCTTAAGCCA
GGCATGGTGGCTCATGCCTGTAATCTCAGCACTTTGGGAGGCTGAGGTGGGTGGATCACCTGAGGTCAGG
AGTTTGAATCCAGCCTGGACAACATGGTGAAACCCCATCTTTACCAAAAATACAAAAAGTTAGCCAAGTG
TAATAGCAGACACCTGTAATCCCAGCTACTTGGGAGGCTGAGGCAGGAGAATCGCTTGAACCCAGGAGGT
GGAGGTTGTAGTGAGCCGAGATTGCACCACTGTGCTCCAGACTGGGCAACAAGAGCGAAACTCCATCTCA
AAAAAAAAAAAGTGTTCCTTTGATAAAGACAGTCCCTGTCCTTGCCCCTACCCTCAGCCCAGGGTTTTTC
AAAGACATTATTGCCATTTTGTCTGGACTAAATCTCTGTTGGGGGCGGGGGTGTCCTCTGTCTGGTGGGG
TGTTTAGCAGCTTCCCTAGCCTCTACTCACTAGATGCCAGCACCTCACCCTAACCTTGTGGCAGCCAAAA
ATGTCTTCACACACATGCCAAAATGTCCTGGTGGGGAGTTAGCAAAATCGCCCCAGTTGAGAACTACGGC
CTTAACCTGCCTCAAAAAGCTGCTCACAGGAGAGGACTGAGGAAAAGCCGGAGGCAAGGTGGGGGTTCTG
CCCAGAAGGAAAGCGAAGATTGGCAAACTCGGAGGGACCCCAGGAAGTGGTGGGCAGGATTTGAAGACCC
AAAGTGTGGTATACCGGGTGGGGGTAGGTGGACAGTGGGTGATAGGACCAAGCTGTACTGAAATCTAGAA
TGATACTAGATTCACAACCCTCCTGTGGCCCCCATCTGGCCCCCCACGGGAGGAGAAGTGATGGGTAGTT
ACTGCGGCTTTTCCCATTCTGAAATTGTCACCTGCAGCTGCACCTGCTGGTGGTGCCTTCTAGAATGGTC
GGCACCCCCAACCCAGCCCTCTCCCAGATAATGGGCAACATGAGTATCTCGGGGGTGTGCTGTGTCTCTT
TCAAGATGACTGTCAAAAGCAAACAAAAGCAGATTGTTTAACAGACATGGCAGCAAAGCTGAAACTCCCT
CTAGCTTGAGTGCAGGAGGTTCTCACTTTTAGCCTCGTCTTTGCCATTCACGAATCAATTGTGAATATTG
TCTGAGCACCTGTCAGGTGCCCCACTCCCTGCTAAGTCTAGCCGGATCGAGCAAAGTCTCTGCCCTCCAG
ATCGAGCAAAGTCTCTGCCCTCCAGAGGTCAACAAGCTGTGGAGACAGGGGAAAGGAGGGGAGGAGAGTG
GCTGGGAGGAGGACATGGAGTTGGGGGCAGAAGAGGCCAGGCCTGGCTGGGGTGCCCCTCCCTCCTGGGC
TTCCAGGCTGAGGTTTCTGCCCTTCTCACCAGGGATGGCTCCTTCAGTGAGCACCCAGCTCTTCCCCAGA
GACTTGGACAGTTGCTTCAGGCCATTTGACTGGGACCTTTTTCCTTCCTCTGGATGGTTTTGTCCTGCTG
GTCACCCTGAGCCCCAGAGAGCCCCATTGAGGGGCCACACTTAGCTTGAGGGAGTTCTTGATGGGTAGGA
TGAATCAAGGGTTGAGCGTGGCAGAAGTCATTGCCCTCCCCCCCAAATCCCAGAACTTTGGCTGAATCAT
CTCATCCAGAACCTGAGAGAGCAGGTGAGCAGGAGGGGATGGTTGGGCAGAGGATGGTGTGGACCAGATG
CGGGAGTGGGGATTTCAGCGGGTGAGCTAACCCGGGGCTGGAAATGTGCAACATGCCCCGCTCGCCCATC
ATCTGCCCAGCATGCCCGGCTCCCACCTGGCTTTGGGGGCATCTCTGGGAGAATGGCGCATCTGGTGCTG
TTGCAGACTGGGGTCCGCGGGCAAGGCCAGGGCAGGGCTTACTCTTGTGCATGAAGGACTGGGTTGGTCG
GGAGCCTGGGGCTGTGTTGGGCCTCAGAGCCGGCCCTCATAGGTCTCTATGCTACGGAGCCAGCAGTCAA
CAGGAAGTGAGTCCCAGCATGAGAGGAGGCCATGTGGTCCCAGGCAGGAAGTGAGTGACAGTGGAGTTGC
CTCCAGCAGCTTTGCGGGGCTCAGAGCCTGCCCCAATCTGAGGTGTCTGCAGGGCAGCAAATGATGAGAA
GAAAGGAAGACGCAGCCATGAAAAAGTCTGGCTCTGACCTAAGCTTCTTAGCTCAAGTTAGCCAGGCAGG
ACCCCACGCATACCTCTATGGTAGTGCCATGTTGGGGGCTTTGGGGTACCTGGAACAGCTGGATCCCAGA
AGGTGGCTGCAGTGCAGGGGTACACTCCACTTTTCAGGCTGCTGGGGCTCATGACTGAGCCTTGAAGGGT
GACAGACAGGGTTTACACCAGGGAGGACCATTCTAGGCTGGAAATGCCCTCTTTGCTTAAGCAGGGAGGG
AGGAGAGGTGTTCTGAGTTCTGGGGAGGGTCCTGACACACAGGATGGAGGCAGGGGTCTGCCGTTCCACT
TTCCTTCCCGGATTTGGCCTTCCTGGCTCTGCTGGCATCAGCCCAAGCCCCCAAGCACAGTGGAGGACTT
CTCGCTTTTACCAGTCACCTCCTCTTCCTCTCGTTGCAGCCCCCTGCCTCCAACTGCTGAGGAGTACACA
CATGCAGCTTTCTGTGGGTGCTTCCCTCCTGGAAGCAGGCCTGCCCAGCTGCCCCGCACAGCAGGAAAGG
AGCTTGGCAGGCCCTGCAGTTCCTTCTGCTCCCTCTCCGGCTGCTCCCTGTGCCCGGGTCCCCATCCAGG
GAGGAAAGCATACCTGAGAAGACTCTGGTTTTGTTTTTGTTTTTGTTTTCCCAAGGCCCTTGTCCCTAAT
TGCCACCTCTGGGCGTTCCTTGCCAAATAACAGTGGGTTGAGAGTTCAGGTCCTAGACCGTTAGAGCAAA
AGCAGCCTTGGAAATAGGGCTTTGCAGGGGAGGGGCTGGGGACCCAGTGCTGTCTCAGGCTGTGCACTTG
AGTGGTGGGAAACTCTGGAGGGAATTTGGCAGTATCTCTGCACATTTAGCTGGACAGGCCCTTTCACCCG
GCCTGACCACTTCTGGAACTCCGTCCCATGGAAACACGCAGCTGTACAGAGCTGTAAGTGCAAGGGTGTT
CACTGCAGTGAAAAGCAAATCAGCCCAAATGCCCACCATCAGGGAACGGCATGCATACATTTTGGAGCAT
CCAGATCCATCTGCACTGGGATGGAGGGATGCCCACTCTGCTTGTTAAGTGAAAAAGCAAGTTGCAGAAC
ATTACGACTAGTATGATACCCTTTTGGAAAAAAACATTTCTGGATCTGAATGTGTATATGAATACATATG
TATAAACATGTAACTTACACATGATTGTCAATGCATAGGAAAAAATCTGGAAAGGATGCTCACTGAACTG
TTAATAATGGGTACCTTAGGGGAGAGATTTTGGGGGTAGGGAGCTGAAATAGGAGCTTTAACCTTTTAGC
ATTATTTTTTTTTTTTTGTATAGTCTTTTTTTTTTTTTTTTTTTTTTTTTTTTTTAGATGGAGTCTCACT
CTGTTGCCCAGGCTGGAGTGCAGTGGAGCAGTTTCAGCTCACTGCAACCTCTACCTCCTGGATTCAAGTG
ATTCTCCTGCCTCAGCCTCCCAAATAGCTGAGATGACAGGCATGCACCACCACACCCAGCTAATTTTTAG
TAGAAACAGGGTTTTGCCATGTTGGCCAGGCTGGTCTCAAACTCCTGACCTCAGGTGATATGTGAGGGAG
CCTGCCTCAGCCTCCCAAAGTGCTAGGATTATAGGCGTAAGCCACCACGCCTGGCTACAGTTTGATTTTT
TTATGATATATGTGACTGACTTTTATAAGTAGAAAAAAAAAGACAAGAAAGACAAAATCTCCATTGGTCT
AGACCAGTCATCTCCACCTGACAGGTGGGGAAACTGAGATGCTGAGAGCAGTGCCTTCCTTATCTGAGGC
TTGCCGTGCTGATGCTGTCCCTGCCTTTCTACTGGGTAGGTGTTCTGCATACGGAGCCAGCGAGGCAGAG
TTGCTGAGTGCAGCCTGGTGCCTGTGGGTGTGGCAGAGTGCCGAGCCATGGGGACCCCTGCCTGTTCCTG
TCCCACACTGGACACCTCCATTCTTCTTGAGGTGTCCTGGCCCACTTGCCTCTGGATGGCTGAGGACTGA
ATATATATACACAGCAGAGCACACAGCAGGCACCCGGCTCTCCTGCTCTTGCTTTGTCCTTGCAAAGCCT
GTGACCTGGAGCAAATCACAAAATTCCAGAGAGCCTCAGGTTCCTCACCTGCTTCCCAGGGAGGCTGAGA
AACTCTGAGGAGGTAGTAAGTGAGGAGCTTTGTCCACTGCGCAGTGACACACACAGACAGCCAGGATTAT
TCTTTATTTGATTTTTATTACCTGGAACCTTAGCGAGTTCAGTTCTTAGACTCTAGGGGATAAAGAAATG
ATTTCCCTTGAAGGGAGAATGAGAGACCCCAGTGCTCAGGAAGAGTTGTTGCAGAGCCTGAAAACTGTTT
GCCAGAGCCTTGGCTCTTCTGGGTCTGAGTGCCTGAAAGAATCTGGGGCGGCCCAGGCCAGGGGAGAAGG
CTACGCTAGGCTATGAGCCTACAGAATGGTTTTTTGGGGGTCCTCCTTCGACTCTCATGCCTCAAGAGAC
CAAGAGGAAAGCTGGGAGGGCACTGACCACAGAAGTCAGACCAGCCTGAGTCCCAGACTTGATCTGGTGG
CACTGGAATCTTCTAAGGGAGAGGGGGGTCTTATTGGATTATGGGTGGCACAAGGAGCTTTTTTTTTTTT
TTTTTTTTTTGAGGCGGAGTCTTGCTCTGTCGCAAGGCTGGAGTGCAATGGCGTGATCTCGGCTCACCGC
AAACTCTGCCTCCCAGGTTCAAGCGGTTCTCCTGCCTCAGCCTCCCGAGTACCTGGGATTACGGGCATGC
ACCACCATCCCCAACTAACTTTGTATTTTTAGTAGAGATGGGGTTTCTCCATGTTGGCCAGGCTGCTCTT
AAACTCCCGACCTCAAGTGATTCACCTGCCTCAGCCTCCCAAAGTGCTGGGATTACAGGTGTGAGCCACC
ACGCCTGGCCGGAGCATTTCTTTTTTAACAGGGGCTGCAGAGAAGGCTTGGACGTCCTATGTAACCTCAG
TGAGTGGCGGTGGGATGGGGGAGCCGATCCCTTGAGAAGTGCAACCAGGCTGGCAAACCTGCTTGGATTC
TTTTCCTGTTTCTGAAGCTGGAGTCCACAGACTCGTGATTTCAAGGGGTCAAGCCCAGGAATCCTTTGAT
GTTGAAAGCAAAATTGTGAATGTATCTGTTTGCATGAAGGTTTATATGAACACAGTTTCTGGACAGAAAG
TGCATAACATTCTCTAGAATTTCAGAGCGGTCAATGATCCAAAACAGGCTAAGCCCACTGATCTAACCCT
CCTCTTGTGTATCTTTCTTACATTTGCAGGTGGAAAGCGGTGACACGCCAAAGGACCCTGCGGTGATCTC
CAAGTCCCCATCCATGGCCCAGGACTCAGGCGCCTCAGAGCTATTACCCAATGGGGACTTGGAGAAGCGG
AGTGAGCCCCAGCCAGAGGAGGGGAGCCCTGCTGGGGGGCAGAAGGGCGGGGCCCCAGCAGAGGGAGAGG
GTGCAGCTGAGACCCTGCCTGAAGCCTCAAGAGCAGTGGAAAATGGCTGCTGCACCCCCAAGGAGGGCCG
AGGAGCCCCTGCAGAAGCGGGTGAGTCCTCAGCACCAGGGGCAGCCTCTTCTGGGCCCACCAGCATACCC
TGAGAGTCAGGGACTTGGCTCTCCAGCAGGTCCCAGGAAGGATGGTCTGGGTCGTGGCTAAAGGTCTGCT
TGCCAAGGCTATGGCCTGGAGGCTACTGGCTGGATGCAGCCTGCGCATATGTTTTATTTGGCCCATAGAG
TGTTTTAAACATTTAAAAAATTAGTTGCCAGTATTTAAAAATCAAAAAATTTCACATAAAAATCTGGAGT
TTTGGCTTCTCATGAAAAAAAAAAAAGCTAGATCTGGCAACAGCGGGCTTTCATAACGCCAACGATTGCT
AGACTGGGATAATGGCGGTCCCTCCATCGCCTTCTGTGGCTGGTTGTGGGCCTTAGTTTTCTGCAGCTCT
ACCTGGCCTGCTTACTCTCCCACGTGCCATGCAGTTCCTGGGGGTTGCTGTATTTGTAGCCCCTGGCCTG
GGCACTCAAGGGCAGCAGATACCCTGTTTGCCTCCCTGAGTGCAGAGGTCCTGAGCCCACCCTAGTTGGG
CTGACTCAACTGGAAATTTGGTTGTGACAGTGGCGTGGGGAGAGGGCTGGGTGATTGTATTCTGTGTACT
GCCCAGCCCAGGCCTCTTCATCTGGGGACTTTTTGGCCTAACCCTGGAAGCCTGGAAAGTTGCCCACTTT
TCTCTTTCAGGTTAAGCCAGCAATTTCAGGGCCAACCGAGCTGTAAACATGTTAGTAATGAGGACAACTA
GCATTTGTACAGGGCTTCACAGTTTACAAAGCGCTTTCTCATACATTATCACATTTGATCCTCCCAGGGC
CCTGCCAGGTTGTTTTGCATATGTGCATTTTAATTTCAAAAAGTCTTCCTTCCAAGCGTGTATGATGAAA
TGAGTAAATTGATTAATTGGCGTAACTTATTTTGCATGGATCCAACCTAATGTTCATGCAGGATAGAGAA
CATTTCCAGAATACAAATTTCCAAACTTATTAAGAAGCTTTTATTCATTTATTATTCGACAAATATGTAT
GATGCTCCCACTATGTGCAGAACACTACAGAGGGAATCTGTATTAGTTGTTTTATTTACTCCTCTAACAA
TCTTATTCAAGAGATATGATTATCCTCATTTCAGGAGAAAAATATTGAGGCTTAAAACAATGATGTGACT
TGCTCATGGAGCCCCAACCTTAAGTGGCAGATCCAGGGCTCATTCAGTCCTGGTCTTGTGCCTTTCTACT
ATTTTCCGCTGTCCTGGAATGGGACTTGGTGCGGAGACGGGTCCTGAATTGTCTAGGCCTCTTGCACATA
TTCTGATACTTCCCGAGAGTGGGAAGGGCCTGAGGGGAGTGAAACAGATGAGGAAAGGGAGAAGACACCA
TCCTTCCCATTAACAATGCCAGGTGTGTGAGAGAACCCATCAGCCCTGCCCTTCCAAATGTGGTGCTTCC
CATGGGCTGACTGCAGTTGACTTCGCTTCCCAGGACCCCACCATGACTCCTGCCGAGCTGCTACCCAGGG
TCACGGCATTCCTTCCTGCCCTGAAGCATGGAGCCATAGCCCCAAAGCTGTTCTCTGGCCTTGAGGGTTT
GTTTTCACTTTGGGATTTCCCTACTTCCATTTCTAGCTGTTCTCAAGGGCTCCTGCATCGATCAGTCAAC
AGGTGTTTCTTAAAACACCCATTCAGGGTGTGCTAGGATCCATACCAGGGACTGGGGAAACTATTATGAA
CAAAAACCTGCTTCTTGCCCTCAAGGACCTCACAGTCAGCTGGAGAGACAGACATTAATGAGAAATCCAC
CCTTAAAAAGATGAGATTACACATGGAGATAAGCGCTCTAAGGGAAGTGAAACATGTCCAGGGGAACTGC
AACGAAGAACCTGAGTTGGCCTGGGCTGTCCAGCGCGAGCTGAAGGGCGAAGGGAGTCCACTGGGTGAGG
GGTAGAGGGAGAGAGTGAGAACAGGGAAGAAGGCTCCCAGGTGGGAGGGAGCAGGGCACCCAGGGAACTG
ATAGATGTCACAAGGCTAGAGTGCAGAGAGCAAAGGAGAAGGGTGTGGAAAGTGCTGGGGCAGACAGGCC
AGGCCTTCAAAGGTGGCTGCCGGGGTGGGGGGTGGGGGGGGGGGGAGGCGGGGGGTGGGGAGGCGGGGAG
TGGGCAGTGTGTGTGATGTAATCAGCTTTGCAATTTGAAAAGCTCATGTCATCTGGATCCTGGGATGGAA
TGGAGGAGCTGGGAAGCAGGATGACATCGGAGGGGCTGATGAGCAGGATGGCAGCAGAGATGAAGGGAGG
GGACAAATGCCAGGAACATGAGAGGTGAGGGAGAGATGCTGTCAAGGATGATTCCTGGGTCTGGGACTTT
CAGACAGGTGGATCTTCCTTAGAATCTTAGTGCTGAGTGTGGTGGGCAAAAACTCTTTAAGTCTCCTTGA
CCTCCAAAATTTGGCCTTGAGGCTGGGCACAGTGGCTCATGCTGGTAATCCTAGCACTTTGGGAGGCCCA
GGCGGGCAGATGACTTGAGGTCAGGAGTTCGAGACCAGCCGGGCCAACACAGTGAAACCTTGTCTCTACT
AAAAATACAAAAAAAAATTAGTCGAGCTTGGTGGTGCATGCCTGTAGTCGCAGCTACTCCGGAGTCTGAG
ACAGGAGAATCACTTAAACCCGGGAGGTGGAGGTTGCAGTAAGCCAAGATTGTGCCACTGCACTCCAGCC
TGGGCAATAGAGTGAGACTCCATCTCAAAAAAATACAAAAAAAAAAAACAAACTTTGGCCTTGGTACACT
CCTGTGTGGTCCCAGCTATCCAGGAAGTATAGATTCTGGCAGGGATCAGAGCTGGTAGGATCCCTGAAGG
CCACTGCACTGCCTGGAAGCTGAAATTGGTGAGATGTTACCTCGCGGGGTGGGCAGTGGCCTAGGCTCTG
CCAATATGCAACGGACAGTGGCTTAATCAGGGTGTGGAGGCTCGGTCACGGTTCCAGCAGTCAGCTGACA
TAGAACACATGCTCAGCTGGTTGATTCAGATGAGGAAGGAGACTAAGGCCCCGGAGAGGATGTGGCTCGG
CCGTGGTCTCCTTTCAGTTCCCCTTATTCCAGGTCTCTTTTCAGGGGATGCTCTCCTGGAACCCCTTTCA
TTAGCTCCTGTACAGCAAAAACAGACTTTTTAAGGCAGCGTTTCTTGTAGCCTGAGCATGTGCTGAAAGT
ACAGCCAGGAGAAAGAATCAGGAAGACGGGAGATGATGAGATGAGTGATAGGGACAGGAGCCAACACCCG
ACAGGCCTGTGCTTGTCTTGGGTCTGTTCCTGCCCCCGCCAGCATTAATGCCATAATGATGATGATTCAG
GTGGCAGTGGCGATGTGGCTTTCCCTTATTGAGTATTGACTGTATTGGGCATTCTCTCTCCTCGCTACTC
ACAGTGGTCCATAGACCAGCAGTGTTGGCAGCACCCAGGAATTTAGAAATGCAGAATTCCAGGCTCCACT
GAAGACCTGCTGGATCAGAATCTGCATTTTAAAGAGGGCCTCATGCTTCCCATGTATATGAAAGTTTCAG
AAGCTCTGCTGTGTATTATGCCCCATGTGTGATCACGGGAGGAAGATTTTTTTTTTTTTTTGAGACAGCG
TCTCATTCACTCTGTCGCTCAGGCTGGAGTGCAGTGGCACAATCTTGGCTTACTGCAACCTCCACCTCCC
AGGTTGAAGCGATTCTCCTGCCTCAGCCTCCGGAGTAGCTGGGATTACAGGCGCCAGCCACCATACCTGG
TTAATTTTTGTAATTTTAGTAGAGACAGGGTTTCGCCATGTTGGCCAGGCTGGTCGTGGACTCCTGATCT
CAAGTGATCTGCCCACCTTGGCCTTCCAAAGTGCTGAGATTACAGGCATGAGCCACTGCACTCAGCCTGG
AAGACTCTTATTATCCTCATTTTTCAGATGGGGAGACTGAAGCTTCAGAAAGGTTAAATTACTTACCCAA
GATTATGCAACTGGTGAGAGACCGGATTAGGGTCAGCACAAAACCCCTCTCTACTCTGAGCATAGGGCCC
CTTTTCAGGCCAGTGACTGCCCTCTGAGGTAAACCTGGCTACAGCCTGGTCATGGAGATTTGAGTTCATT
TTAAAGAGAACTTGGGTGGCCTCTGGCTGAGTATAGCTCAGACTCGAAGGCGAGTGTTAGAAGCACCCTG
GCCTCCCCTGCCCCCAAGTCCTGAGATGCACTAGGGTCATGTTCGGGCCTGTCAGGGCCACACTGGAAGG
TCACAACCACCTGGCCCTGGCGGTGACCCTGGGCCTGGGCCAGGAGCAGTTGGGCTGCATAGGCCGTACT
GAGAGAGCAGCTGACCCTCCCTGACTCACGACAAGTGCCGCATTCCGAGGCGGCCCGGCCAGGCTGGAAA
GCGTGTGTGTCTGTGTGTGTGTGTGTGTGTGTGTGTGTGTGTGTGTGTGATCACTCAAGTGGGCATGCGC
AGAGTCTGTCAGGGAGAAAATTCCAGAAAGAAGCCAGTTGGAAACTGCATAGGGAAAAATCGTTCTGTTT
TGGGGAGCTTGGGGGGCTGGGAAATTAGGAAGGGGGTCACATTGAGAAATAACAGTAGTGGTGCAGCGGA
GAGGGAGCTTGCCGGGGCATCAGGACCCCTGAAAAGCTCATCTCAGCTCCGGCCTTAAGTCACTCCACCC
CCCAGGTCTCAGCTTTCTGCAAAATTTAAGTGCTGAACAAGTACTCAATGATCTCTGCAAAGCCTTTCAA
CCCTGATTTTCTAATATTCTAATGAATAAATAATTATCCGTAAAAGTAATTAAATGCCATGGAGGGTCCC
AAGTAGGAAACGTTTGAGGTTCTAAAGCGGGCTGGGGAGAAAATGCAGTCGGAAGCCCCAAGTCCAGGTC
CGAGAGCCAGCCCGGGGGTCCTCCACTCCCCGCCCTAGCCGCAGGGCTGACACAGTGGTTCCCAGCGCCA
CCGCGAGCCCCGGAGCCCCGCCGCGTCAGGGTCCGCCGGGACCGCCAGGCCGCCCCCAGGCCGCCACCTG
ATCGCGAGCAGGCGCCCCCTGCTGGGAGCCCGGGGCCTCACCGGGCAGAGCAACGAGGTCTGGGAGGAGG
GGGAAGCCCTGGCCTGGGAACGGGGAGGCCCCGGACAGGCTGGAGGGGGCCCCCGCGAAAAAAGTGGGGA
GCTGGCTGTGGTGGGAGAGAGGACGGAAAGGAAACAGGGAGGCAGAGCCAGACAAGATGGGTGAGACAGC
CCTTAAAAGGCCGGTCACGAACAAAGCGCTGGCGAGTGCGCGCCCGCCCACGCGCACAGGTGCCCGCGAC
AAGACGCCCCGTCCCCGCCCACGCGGCCCCCGCGGGCTGAGCCCGGCGCCAGGCCCGCACCCACGGTGGC
CCTCCTGCCTCCCTCCCCCTGGTGCTCCCGGGCCTAGCTAGCGGGAGTCGCTGTCACCAGGGACAGAACC
ACGGAGGACAGCGCTGTCCTGGGCGCCCGGGGTCAGCCCAAAACCAGTCCAGGACCCCCCTGTCACGAGG
GAAGGGAGTGGCCTCCCGGGCTGAGGACCCGCGGCGTGAACACTGTGGCTGGCTTCCGCTTCACAGCCAG
GAGATGAAGTCGGGGCTGTTTTTTCCGTTTCAGGCTCTTTGTCTCCCCCCACCCCAAGCTACCATGAGCC
AGCCTTAGTGACCTTCCTGCAGTGGGCCCAGGCCCGATACGCTGTCGGTTCCCGACAAAGAAGCTCAGGG
GCCCAGGTGGAAGGAGGGCTCACTTCCCCACCTCCAGCAAGAAGCTATGGGAGGCAGAATCAGCCAGCCC
CGCAGCCTCCGGGGCGCTGGGCCTGGAAGGGGTGCCTGGAAAGGGTGGTGAGGACCTGGTGGGGAGTGCA
GGGGGATGCCTGGGAGGAGCGCAGCTCCCCTCATGCTGGGGGCTTGGAGAAGCACAGGGAGAGGGCAAGG
TCTCAATAGGGAAATTGAGACTTCCCTGCCAGGACCTGGAATCAGGGCCCAGTAGAGGAAATGAATCCTT
AGGGAAGGAGAACCACACTAACATGTATTGAGCACCTACAGTATGCCAGGCCCCACAGTAGGCACTTAAT
GCTCACCATAACCTAATGATTTTAGAACATTTATCTACCTTGATTCAGTGGGAAAACAGGCCAGGAGAGG
TTACAGAGGTCACACTGGAATCCAAACTCAAGTCTTCTAGATTAAAGGCTGAGCTCAGGGGCCTGAGCAT
GGGGCAGGGAGTGGAGGGACGGAGAATGAGATTAGTAATAGAATCATAATATTTGGACCTGCAGGCGGAC
CTTGGAAATCGCATCCAGCATCCTTATTTTCCAGATGAGGAAACTGAGTCTCGTAGAGAGGAAGTTGCTT
CACCGGGGTCTTGTTCCAGTTGGCCCGGTACCCACCGCCCCCCAGTACATGCCCTTATGCCCAGAAGGCC
TCTGCAAACACTCCCTTGCAGGGCCAGGGAGGCCGCTCAGGCTGGCCCTCTGGCCCAGGCCTTCCTGCAC
CCCCTCCTCCTTCCGCACCCCCTCTGCCTTCCTGCTCCTCCTTTGGGGAAGGAGGTCTCTTGACTGGGAG
CAGCAGAATGGGGGTATTTTTAGTCCCCTTCTGTGTACCTGAGACAGAGTTGACAGGCCAGAAACCCAGC
TCTGAATTTCCTCGTGTTCCCTCTTGCCTGGGCCTCAAGGCCTGCCCCTGCCCGAGTTCTTTCTCTGCCC
TAGGCCCTGCTGGGTCCTCAGGGTCAGCACTGTGCCTGGCATGTGGTGGGCCATCATATTTAACACATGA
ACAGCTAAACGGCCAGAGGACCCCCATCCCCTGTGGCAGGGACCCCAGGCACAAGGTGGGGCCTGTGAGC
CACTCCAGTAATTCTGCCGACCAACGAACTGGTCCCTTTGTTCTTCCCTCTCCACAGGCAAAGAACAGAA
GGAGACCAACATCGAATCCATGAAAATGGAGGCAAGTGTTCCACCCCCAGGGGCTCTGTGGATCCTGATT
CTAGAAGAAGGACACGGGTGGCGAGTGTGTGTGAGGGGGCGGAGGGTGGGCCCCTCCTCCTTCTTTACAC
ATCCCTCTGTGGGAAGGAGAGGAAGTGGGCGTGGAGGTTGGAAGTCCCCCAACAAGGCAGCCGGCTCATC
CCCCTCCAGAGGCCAGACGCCTCCCTTCGTGGCCTGTGGCCGTGTCCATGTGCCCCCCCACCCCCGCCAC
ACATGCCCCTCGCTCCTCCAGCCCACACGGGGCCTGCCCAGCACTCCCAGGGGTGCCCTCTCTGGCCAGG
AGCCAGTGTCCACATTGTGTTTGAGGCGAGTGCCTCCTTGGTGGGTCCGTCAGTGCCCACCCTAATGCCC
TAATGTCTGTCTCTCTGTCCTAGGGCTCCCGGGGCCGGCTGCGGGGTGGCTTGGGCTGGGAGTCCAGCCT
CCGTCAGCGGCCCATGCCGAGGCTCACCTTCCAGGCGGGGGACCCCTACTACATCAGCAAGCGCAAGCGG
GACGAGTGGCTGGCACGCTGGAAAAGGGAGGTGAGGCGCCTTCTGGCCTGGGCCCCAGCCCGTCCTGCAG
AACTGGAAAACCAGAAGTGATGGGCCTCAGTTTCCCCTTCAGGCTAACCCTTAGAACTTACTGGGAGCCT
CTTAATCTCTAGTCATGATGACTGGTTCATCTGCTCCTTCAGCAGATGCTTATTGAACACTTACTGTATA
CCTGGCTGTGTACTAGACCCTGGGCGTATACGGTTCCTGCTCTCATGGAGTTGACATTCTGATGGGGGAG
ACAGGCAGAACATAAAGAAACAAGGGGCTGAAATAGAAGATAGCAGAGGCCTGCTTTAGTTGGGGTGGTC
AAGACAGGCTTCTTGGAGGAGGTGGCATTTCAGTTAAGGTCTGAAGGAACCGAAGAGCTGGCTCTGTGAG
GAGTGGGGTCAGGGAGAGTGTTCTAGGCAGAGGAAACAGCACCAGCAAGGCCTTGACTTGTTTGGCAAAC
TGTATGCCAGGGTGAAATGGCAAAGGAGAAAGGGCCAGATGCAGCCAGAGAGGCAGGCGGGGCTCCACAC
ATCAGTTGGAGACCTGCAGAGTCTGCCCAAAAGCTGGCTTACTTCTGACAATACTGGGCAGCCATAGAAG
GTTTCTGTGCACAGGAAGGATGTGATGTGGATTTGAGAGGCAGCAGTGGAGACAGAGAAGAGCTTTGGGG
TGTATTTTGGAGCTAGAATAGCTAGGGCTTGCTGATGGACCGGCAGGGAGGGGAACTGGTGGACTCAAGG
AGGACTCACAGGGGTCTGGCCTGAACACCTGGTGATGGTTGTGCCATTTGCTCAGATGGGAAGACCCAGG
GGAGGATGAGTTTGGGACAGGTGGGAATTGAGGGCTCCGTGTGAGATGCCAGGTGCCTGGAGTCTGGGCT
GTAGGGAAGTACGGGCGACAGAGGTGGGATTTGAGAGCAGAACCCTGGCATTTAAATCCATGGGAACTTA
AGGGCTCAGCAAGGGGAGAAGGAGGAGAGACGAAGTCTCAGAACATGTCCATGAAAACCCCGACATTTCT
GGGCCACATGGAGAGGAGGAGTGGCAGGCAGTAGGGACTGAGGGAGAAAGCAGTACAGAGCGGTGTCTGG
GGAAGCACGGGGTTTTCCCAGAGGGTGGCTGCCCAGCCCTAGGGAAAGCTGAATTCACTGAGACATGCCC
ATGGCGTTGGACAACCTGGAAAGGAGCTGGTGACTTTATAGAGAAGCTTCTGTGGGGGTCGGGGAGGTGG
GAACAGCCCGTGGAGGGCTTCAGCCATGCAGAGAGGTGAGAAAATGGAGGCTGAAAGACTGAGGAGGGAA
ATCAACTCGCCACGCACAATTTTTGAGCTGTCTTGTATGTTTGATTAGTAGAGAACTGGGTACAAAGAGA
AGTGGGTGGTGACCTAGCTGCACAGGAAAAGAGAGGAAACTGCCACCACGTGGGGCACTTCACGTGAACT
CAGCTCTCATTTCGTCTTTACCCCAGGCCTCCAGGGGAAGGCGGCCCTGTTTGTCTGTTCGCCTTTGTGT
GAGAGCTTTCTCTAGAGAAAGTGCAGAACAGAGGCCAGGCGCGGTGGCTTACACCTGTAATCTCAGCACT
TTGGGAAGCTGAAAGGAGGTCAGGAGTTCGAGACCAGCCTGGCTAGCTTGGTGAACCCCTGTCTCTACCA
AAAATACAAAAAATTAGCCAGGCGTGGTGGCTCATGCCTGTAATCCCAGCACTTTGGGAGGCTGAGGCGG
GCGGATCACCTAAGGTCAGGAGTTCGAGACCAGTCTGCCCAACATGGCAAAACCCTGTTTCTACTAAAAA
GACAAAAAATTAGCCGGGCATGGTGGCAGGCGCCTGTAATCCCAGCTACTCAGGAGACTGAGGCAGGAGA
ATCACTTGAACCCGGGAGGCGGAGGTTGCAGTGAGCTGAGATCACACCACTACACTCCAGCCTGGACAAC
AAGAGCAAAACTCTGTCTCAAAAAAAAAAAAAATACAAACAATTAGCCAGGTGTGGTGGTGCGTGCCTAT
AGTCCCAGCTACTCAAGAGGCTGAGGCAGGAGAATTGCTTGAACCCGACAGGTGGAGGTTGCGGTGAGGC
GAGATTGAGCCACTGCACTCCAGCCTGGGCAACAGAGCAAAATTCCGTCTCAAAAAAAAAAAAAAAAGAG
AAAGTGCAGAACAGGGTGGGGGTTCTGGCTGCTTGATTGTTTTCTTTTCCTCTGGGATCCAGGAAGGCAG
AAATTACTTTCCCGGAAGAGCCCGGAGAAAGGGCAGCCTGTCTGAGGTCTGGGAGGAGAGGCACTTACCC
CTGTGCGTGGAATGTTGTGTTTGGGGTGGAAATTCAGGAATGGGGCTCTTCTCTCCAAGTCAGTCGCTGC
ACTCCTGGGTTCCCCGCAACCCAGAAACAGGGGCTTTGCTTTTTAGCTCCTGGTTCTGTGAGCGAAGTGT
TTCTGTTCTCATTTAGCCCTGGGGCAAGAAGCTTAGACTATGACTTTTAGGAACCGGAAGAACTTTCAGA
AATTCCATGATTCTCAAACTCTGCTCCCTTGAAGAAGCAGTATGGAGGCCTGCCCTCAGTTACAGTGAAA
AAAAGGGTTCTTTCCTCAACTCCCAGGAAAAATTAGATCATAGATACATTTTCTCAATTTTAGGTCTCTA
CTAAAATTTAAAAAGACTTTTTTGGGCTGGACGCAGTGGCTCACACCTGTAACCCCAGCACTTCGGGAGG
CCGAGGCGGGTGGATCACCTTAGGTCAGGAGTTTGAGACCAGCCTGGCCAACATGTTGAAACCCCATCTG
TACTACAAATACAAAAATTAGCTGGGCATAGTGGCGGGCACCTGTAATCCCAGCTACTTGGGAGGCTGAG
GCAGGAGAATCACTGGAACCCAGGAGGCAGAGGTTGCAGTGAGCCGAGATTGTGCCACTGCACTCCAGCC
TGGGTGACAGAACGAGACTCTGTCTCAAAAAAACAAAACAGACTTTGTAAAACTCATGAACATTTTCTAA
CATATGCAGAAGCAGAGAGTAGTGTAACGACCTCCCATTGCCATCACCTGGCTCCAGCAATTATCAAAAG
ATTTTCTAGTAAGTCTTCAACCCTTGGCATTTGCATCCCCACTCCCAACCAATGAAGGTAGCAGTGGGCA
TTATTAGAAAAACCAAAATGTTGATTTAATTTAAAAGACTTTAGTACACTTGACTTGCAATGTGGAGATC
TCCAAACAAAATATATTTTCTGCTAATAACATTATTCCTTATTCCTATGCTGTAATTAAAGAATCCATGA
ATTCCAGTACAAGTTTTAGAACTACTGATCTCATTTTCAGACTAGGGGACTTGGGGCTACCGAGTTTTAT
AACTGGCCACAAGCTCCCAATGAAGAATTGGGAACAGGACTAGAATGGAATTAGAATTTACGTTTCCTAT
TCCAATGTGGGGCCCAGGCCCTGACTGTCACACAGTCTCCTCCTGGGCTATGCCTGGGGTTAGGAAGGGG
CTAGATGCAGGTATCAAGAATGGGGAAAACACAGGTAGCTTTCAAGATTCCCAAGTCGCCCTCAGACTCA
AGAGTAGTAGCTGTTGCTATCAGGCCTGTTTTCTTTGTTTGTCTGTTTTTGAGACGGAGTTTCACTCTTG
TTGTCAAGGCTGGAGTGCAAAGGCTGGAGTGCAATGGCTCACTGCAGCCTCCGCCTCCTGGGTTGAGGCG
ATTCTCCTGCCTCAGCCTCCCGAGTGGCTGGGATCACAGGCACCTGCTGACACACCCGGCTAACTTTTTG
TATTTTTAGTAGAGATGGGGGTTTCACCATGTTGGTCAGGCTGGTCTTGAACTCTTGACCTCAGGTGATC
TGCCCGCCTTGGCCTCCCGAAGTGCTGGGATTACAGGCATGAATCACTGCACCCGGCCTATCAGGCCTGT
TTTTGTTTTTTGGTTTTTGTTTTTTTGAGACGGAGTTTCGCACTTGTTGCCCAAGCTGGAGTGCAATGGC
ACAATCTCAGCTCACTGCAACCTCCATCTCCCAGGTTCAAGCGATTCTCCTGCCTCAGCCTCCCGAGTAG
CTGGGATTACAGGCGTGCACCACCACACCCGGCTAATTTTTTGTAATGTTTTCTAAGTGGTGTAACAACA
GCACAGACATTTCTCCTCTTTTCTCAGTCTCATTTTGCTCTCTGACATTTGAGCCCTTCTGAAACTGCCT
CCCTGGGCCTTCCCAAGGCCGCTGCAGGTTCCTCCCCACCTCCTTCAGCTGATCTCAAGCGGCAACCTCT
GTTAGCTGCATCCCCAGATCTCCACCCCCCACCCTCTGCCAGTGTTGATCCACTGACCCCTTACCCACCC
TCCTAGATCCAAGTACCCTCCTCCTCCCAAAAAAGGCACCTCCCCTCCTGACAAACCACCCAGCCTTCAG
GACCCAGCTCCAGCTGTTCCCCAGGCAATATCACTTGAAGGGCCAGCCAGCGCTTCCTAATCTCAGAATG
CAACGCCTGCAACCTTTCATTCAGGGCACCTTTACTGAGCCGGTTTCCAGAAGCCTCCACAGCTGAGTGA
CATGTAAGATACTGGGACTACCAGAGGCATAGAGGATTCCTAGTTTTCCCATGATAGGATGAGGGCTCAT
AGCACCTGCAGAAAGAGCTGCCCTGAATGGGGCGTAGACGAATGCTGCCTGGGGTCATCTACGCATCAGC
AGGGGTGGCTTCTGGCTTTTGACGGTTGAACTGGAATTTAGGGGAGGGCTGAGGAAGGCATTTCAGAGGG
AGGAATACAGCAGGCACATACCTCTCGAGGCACAAGACACTTGGGCTTTTCAGGAATGGGAGTTTGTTGT
GGTGGTGCACAGCGGGTGTGGGCAGAAAGCAGCTAGGAAATGTGGCTGGAAGGTGGGGCAGGGGCCAGAA
GGAGGAGCTGCCACCAGACCCCAGGCTGCACCAGCCCTTCCCCTAGGCTGTGTGCCCCACCTGGGGGGCT
TCTTCCCTGGGAACGCACTCCCCAGCTTAACTTCCTTCTCCTTGCCCACCCTTGTTGGACCACAGTTACT
GAGTGAGCCTCTCCCAGATTACCCCCTTCAGGCTTTCCCTCCCCCAAGATTTAAGACGACTCAGAATGTC
ACAAAATGAAAGAAATGGAAAATAAGTGAACGGGGAAAATGAGACATTTCTGCAGTCCCTGAGGCTGAAG
TGAGAATTCCTGCTGGAGCTGGGAAGGAAGTTGTTGTCTTCTGTCTGTTCCATCTCCTGTTGCTGTTCCT
TCACCTGCTTACTAGTCCTGTAAGGACTAAGCTCTGTTTTAATCATGATCACACCCTGCCATAGAACCTC
CAATGGCTCCCCATTGTCTCCCCCATCATGTCAGATACCCTCTGGCTATTTCTCAAGTCTTCAACCCTTC
GACCTCCACAATCAGCCCACCCCACTTTCACTACAGTCCTCTAATAGAAGGGTGTCCCCTAGGAAGGGAC
ATTGCCATGCTCCTTCCTTCCTTCCTGTCATCGTTCCTGCTGTGATTTCCCCAGTCCATTCTTCAGGGTT
GAGGCCAAGAGCTGCTGCCTCCTCCAGGAAGGTTTCCAAGATTTCTCCAGTCCACAATGACCTCTTCCCT
TCCCTGAATCCCTGTAACCCTCACTGGGGTTTGTTTGTTTGTCTGAGACAGTTTCACTCTGTCTCCCAGG
CTGGGGTGCAGTGGTGCAGTCTCTTGGCTCACTGCAACCTCCACCTCCCAGGTTCAAGTGATTCTCGTGC
CTCGCCTCAGCCTTCCCAGTAGCTGGAATTACAGGCACGCACCACCACACCCAGCTAATTTTTATATTTT
AAGTAGAGATGGGGTTTTGCTATGTTGACCTGGCTGGTCTCGAACTCCTGACCCCAAGTGATCCGCCTGC
TTTGGCCTCCCAAAGTACTGGAATTACAGGCATGAGCCACTGCACTCAGACTGTCCTTACGGTTAATATT
ACTTAGCACTCAATCATATTTCATCAGGGTATTTTTCACTGTTTCTTATGTGTTGATCACATCTTCCTCT
GTCTGGCCTAATGCAATGGGCAGGGAGAACAGGCCCAGTGAATACCTGCTGGCTGGCTAAGCAGTTGGGA
GGTAGGTGAATGTGGGACCACTTCTCTGGGAACATGAGATCTTAGATTTCCACCATGACAAGGGCCCACA
CACTTGATTTCGAGGTGGTTTTTCCAGGGGTGCTGACAGGTGTACCTGGAGGATGGCATGGGGACTGGTA
TGGACAGCCTCCATGGGTAAAGGGGGGCCAGAGTCTCCTCCATCCTGTATTCCTGCCATTCTCACCAGGC
TTCTGAGCTGAGGAGAACCTTCCTTGGCCTGGCAGAGCTGGCCAGGGCTGTGGGGAGGGCAGGGTAGGGC
AGTTCCTCCTCCCAAAGGAAGGCACTCCCTTAGCTCCTCTTCCTTCTAATTCTCAGAGCAAAATGCACTC
TGATGGAGGGCTTGAGGGTTAGGAAGGGCCTCTCGCTTCATTAGCCCTCCCTGTAGCTCTGCAAGGCAGA
TGAGCACAGAGACCCAGGGTCAGTAGCTGGGCGGTCATCTCGCCTGGGAAGCCACTCACTCCAGTTTGTG
TCGCGGCTGGTGGTTTTAGGACTCAGTTACAGCACATTGATGGCATGACTCGCTCATTAATCTCTTCACC
CCAGCCGGATTCTAAATTCCGTCTAGGCAGCGGGTACCATGTCCTATTGTTCTAGGATCTGGTCTCTTTC
CTGCTTCCGACCTGAAGGTAGGGACCCTTGACTCATGCATCCTTATACTTATTAGATGCTTAATAATATT
GAATAGTGATAATGAAACATCTGTGACTGGAACATCATAGTCTCTCCTCCCTTCCCTCGCCACACTGAGA
TACAACCCACTCCCTCCGCCCAGGGCTGCCCAGCAGATAGCAGAGCCTTCATGGACCTTAAGGGTTGGCT
GCTGTTTACTGCGACCTTTGGAGGCAAGTTCAAATACTGCCTTAAGAAAGGTCCCACTTACTGGGTGTGG
GACCTTAGGCATCACTTACCCTGTACCCCAGCTGAGGTTCCTCCTCTGTGGATGGACTTGGCTGTACTCT
GCATCTCCATGACAGCAGCACTCGCCAGCTTGTTTTCTGCCTCTACCCATTTCTGCATTTAAGCTCGTTC
AATACAGGCATTCTGGGCACCCCCTGCCCCCAGGGCTGGCATGTGGGAGGTGCAGGGTGAAAGTGTGCTG
AATGAATGAACAATTCTTATCTCAGAGTTGTGTGGACAACACACAACATTTAAAGCTCCTAGCACAGAGC
TAGCATATAATAGGAACTTTATAATTTTTTAGAAAGTATTTTAAATTACATAAGTATTACACAAATACAT
TCTTGTGAAAAAGTCCGCCCAAACTTGCCACCCCAAAATTTTATTTTGAGACAGGGTCCTGCTCCATTGG
CCAGGCTGGAGTACAATGGCATGATCATGGCTCACTGCAGCCTCAACTTCTTGGCTCAGGTGATCCTCCC
ACCTAAGCCTCCTGAGTAGCTGGGACCACAGGCATGTGCCACCACACTTGGCTAATTTTTAAATTTTTTG
TAGAGATGGGGGTCTCCTTATGTTGACCAGGCTGGCTTTGAACTCCTGGGCTCAAGCAGTCCTTTTGCCT
CCACCTCCCAAAGTACTGGGATTACAGGCATGAGCCACCACACCTGGCCTGAAAATTTAATTCCTTCACC
AGAAATACTTGATGCTATCAGTTTGGTGTAACGTCATGATTTTTTCCTATCCGTGTGTATGCGTGTAAAT
ATATAATTTTGCTTCAGGGGTTCTTTTTTTCCTGTTCTTTTTACACAAATGGAACCACACTGTAGTTTCA
TTTTACAGCTTGGTTTTTGACTTCCAATAAGTCTGTGCAATCTAGCCACGTAATTACTATTATATTCCAC
CATGTGGATTCCCATGGTTTTATTTAGCCATTCTGTTATTGGTGGCCATTTATGTTGTTTATGATATTTT
ACTGTTTAGCATTGCAGCGGTGAACATCCTTAGTCATACCTCTTTGTGCATCTGTTTCTCTAAGACAGAT
ACCTAGAAAGAAAAAGGCCTAGCTGAAGGGAATGTGCATTTTACATTTTATGACATCTGCCAAACTGCCC
TCTAAAAAGGGCACTCCAATTTATGCTTCTGTGAACAGGGTATGAGAAGACCTTTGCCAACATCAATATT
TTGCTAGTCAAATGGGTGGGAAAATAGTATCTTGTTATGCTTTTAATTGGCATTTCCCAGATAATAAGGT
TGAACATCTTTTCCTATGTTTATTGGCCATTTGTGCTGCTGCTTCTATGAGCTTAGCAAATGTTGCCTCC
TCTGACTTTACAACCCTGCAGTCTCAACAGACTGTGCTTCCTGTCTGCCCTGAGGAGAGCAGGCCATGAA
TGTCCAGAAGGTCCCCCATGCTGTGTCATATTCCATTTAATAAGCAGTCACTCAGCACCTACTAAGTAAA
GGGGCCTTGTGCTAGGGGCTATGGGAAGGAGGAGATAATAAGGTGTGATCTCCTGGGCTTTAGTCTAGTT
CAGGAGAAATGGAAGTGCAAAGTAGACCAGTAAAGGCTCAAAACAAATTATTAGCCAACTGTATGGGAAG
CAGTTTTAGAGCATTTGGACTGGAAGACATTCCTCACTTATTTGCCCTACCTGACCTTTTGCAGCGTGGC
CTCAAAAATAACATCACCCTTGAAGGAGATGATGATGTTTGGAGTAGGCACAGCAAGTATACTTTACCCA
AGGCTGAACAAGTTCTAGAGGCTCCCATGACAGCCTGTAAGCAGTAGTGACTTCGAGGGCTGGCCTTTAC
CATAGGCTACTAAAGTTGCCACAGAGGGACTCCCCATTGTCCTTTAAGTAGATGGGTTGGTTGCGCACCA
GAGTCTTCACTTCTGGGGCACAGAGAGAACAGCTCTTAGAACAACCCCTACTTTTTAGTGGTGAGAAAAA
AAGAGGCAGGGTGGGGTAGGTGACTAGTTGCTGGCCCAGGGCCAACTCCTGGCCACAGCAAAGCCTGAAC
CAGGGAAAGGGTGTTGAACTTCACTGAGCACCTACTTTACATCAGGCACTATGCCAGGAGCATTAGCTTT
CACCACAATCCTAAAACATATTATCTGCATTTAATGTCTTTTTTTTTTTTTTGAGATGGAGTCTTACTCT
GTCACCCAGTCTGAAGTGCAGTGGCATGATCTCAGCTCACTGCAACCTCTGCCTCTTGGGTTCAAGCGAT
TCTCCTGCCCCAGCCTCCCAAGTAGCTAGGATTACAGGCACCTGCCACCACACCCGGCTAATTTTTTGTA
TTTTTAGGAGAGATGGGGTTTCACCATGTTGGCCAGGCTGGTCTTGAACTCCTGACCTCAGGTGATCCAC
CTGCCTCAGCCTCCAAAGTGCTGGGATTACAGGCATGAGCCACCGCGCCCGGCCAGAAGTATTTTTTTAA
CCTGTATTTTGCTATTAGAAAATTAAGATTTATCTAGGTAAGTAGGTTGCGGCCACATGGGTATCCTGAT
GGAGTGGCCCAATGGGCAAAACTAGACTCCAAATCTCGTGTTGCTTCTGTTGTGTACCCAAATGACTAAC
CTACACTCCCAGTTTTCCTGGTTTCCTGGCTTGGAAATTGTCATGAAATTGAAGAATGCATTTAGAAGAG
AGGGAGAGAGCTGGCCCGGCTTTCCAGCCTGCCGCATGCTCTTCCCGCCCCCGGCCTGCAGTTGTTGGGG
CTGCTCCCCATGCCTCTTTGCCAGGAGGCTCAGGTCCCTTTCAGCCTCCCCGACCACCCCTCCCTGCTCT
ACTCATGTGGTCCTGAACCCTTGCCCTTGAATTGCTGTTTTTTCATGACCATGTGGATGATGTTGTGAAA
CTGGATTAAGAAAATGTTGGAATGAGATTTGGGAGGCCTGGTTTCTCGTCTCAGCTTCTGCCCCTCCTTA
GCTATGGATGTGAGGAGGTCTTTCTGCTTTCGGCATTTTAGTTTCCTGGTTTATAATGCGACAGAAATAG
ATGAGATGACCCCAGAGATTCTAAAATTCCATCACGGCAGGTTGGGGGGTGAGACTGGAATCATGATCTG
CACTCTCTTTATTACCTAGATTACCTTGTACATAAATTCTAAATTAAATATTTGAGTGAATTCTCAGACC
TCGCATATACGTCTCTTCTAAAGGCACATACGTGAAGCATCCTTATAAAAACTCTTTTTGGCCGGGGGTG
GTGGCTCACGCCTGTAATCCCAGCACTTTGGGAGGCGAAGGCAGGCGTATCATGAGGTCAGCAGATCGAG
ACCATCCTGGCTAACATGGTGAAACCCCCTCTCTACTAAAAATACAAAAAATTAGCCGGGCGTGGTGGCA
GGCACCTCCCAGCTACTCGGGAGGCTGAGGCAGGAGAATGGCGTGAACCCGGGAGGCGGAGCTTGCAGTG
AGCCGAGATCGCACCACTGCACTCCAGCCTGGGCGACAGAGCCAGACTCTGTCTCACAAAAAAAAACAAA
ACAAAAAACTCTTTTTAAAAAAAAAACCTTTGGCCCAGCACAGTGGCTCACGCCTGTAATCCCAACACTG
TGGGAGGCCAAGGCGGGTGGATCTCTTGAGGCCAGGAATTCGAGACCAGTCTGGCTAACATGGTGAAACG
CCGTCTCTACTGAAAATACAAAAATTAGCTGGGCGTGGTGGCACATGCCTGTAATCCCAGCTACTTGGAA
GGCTGAGGCACAAGAATCGTTTGAGCCAGGGTGGTGGAGGTTGCAGTGAGTCAAGATTGCGCCACTGCAC
TCCAGCCTGGGCGACAGAGCGAGACCTTGTCTCAAAAAAAAAAAAAAAAAAAAAAAAACCAAAGGGTTGT
CCAGCCTTTACTGAGCAGATACTGTGTGCTCAGTCTTTAAGATACCACCTTGTGTGTCTGGGTGTCTACT
ATGTTCCAACATTTTCTAGACACTAAGGAAGCAAATTTAAAGGCAAGTCAGAATTCTACCCTCCCCTGTG
AGAGGTTGGTAGGTAAATGAATGAATTGTGCTGTAGCCAGGGCAGGGTTGTTTTAGAAATACTAAGGGAA
GGACTGTGCAAGCAACTCCCTAGCCCTGCCTAAGGGCCTCAGGGAAGGCGGTGCCTAGGGAGAGGCAGTG
TACCGCCATTGACCTGAACAGGACTGCTTGGCAGGGACAGTGGGGGAAGGGCATGGAGTGGGGTCCTAAG
GGAGGTGGTCAAAGAAATCCTCTAGGAGTTTGGAGTCTAGAGTGCCTCAAAGTTTTTCCCATAGCACTGT
CTATGGGGCTGACAAAGCAGAAAATATAATAGCCAATGTTCTCTCTGCTTTAAAAACCAGACTGCTTGTC
AGGGAGGCCGTGGGATGTTCACATATGGGGTGGTCTGCCAGAGCCCTCTGCCGCTTTATTAAGAGGCCAG
TACACTCGTTAAAGGAGTTAATGGACCTCAGTACTTACTACTGATTGCTTAGTAATTCAGTACTTGGTAA
ATAAACATGATACTATGCTAGTGGCGGGGTAGGGGGAGACCAATTGGATATAGTCCCTGTCCCAGAGGCA
CACATTGTCAAGTGGAGGAGGCAGGAATGTTTCCAACAAGCCTCAGTGGCAGTCAGAGCGTGGGGCTTGC
TCTGTGCTCCTGTGTAAAGAGCCCAACATGAAGCATAAAGGAGCATGAGAGCAGCTTCTGGAGGACTCCA
GGAAAGCTTCCTGGAGGAGGCAGGCATCCTTTGAGAATTTCAGTAAGTGCAAAAGAGGCTGGGTGCAGTG
TAATCCCAGCACTTTGGGAGACCAAAGTGGGAGGATTGCTTGAGCCCAGGAGTTCAAGAGCAGCCTGGGC
AACATGGCCAGACCCACTCTCTACAAAAAAAATATTTAATTAGCTGGGCATGGTGTTGTGCACCAATGGT
CCCAGCTACTCAGGAGGCTGAGGTCGAGGCTGCAGTGAGCCATGATCATGCCACTGGACTCCAGCCTGGG
TGACAGAGTGAGATACTATCTCAATAAATTATAAACAAACAAATAAAAAAGTGTTAAAGAGGAGGAAGGC
ATTTCAGGAAAAGGAACTACTGAGGGCAAAGAGAAGGAGGCATGACTTCTGGGTTGTTTAGGGAGTAGTA
AGAGATCTGGTAACTAGGGTGCAGGGGTTAGGGAGGATGGGCAAGAAGAGACAGAACTAGAAAGCAAGAT
GGTCACATCCTCAGTGGTCTTCCTGCCATAGTGAGAAATTTAGGCTTCGTTGTCCAGTGGAGAGCAGGGG
TGGAAGAGAATTGGACACATAGTTTAGGAAGATAACCTGGCAGGACAATAAGTATTCCCCAGAGAGAGGA
AGTTTAGAAGCAGGACCAGGTAGGAGAACATTACATTTGGCCTTGACTGAGGGTTGAGGTAATGAGAGGC
AAACTTGAAGGAATTGCTAGAACTGGTCCTGGTTGGAGGGGAGGGAAAAATTTGTCTGGTATTGGGAGCT
GGGCTGGGGATAGGGTTCATTATCTGAGGTAGAGAATCTTGGAGGAAGAGCAGGTTTGAGGCAGGATGAT
ACAGTGAACTTTTCATTTTCTTATGTATTCTGATGAGCAGAAGTTTTTAATAAAGTCCAACTTACCCATT
TTTTTCCTTTATTGGTTAGTGTTTGTGTGTGTTATCTCTAAGAATTCTCTGTCTACCGCAAGGTCTTTAA
GATATTTTCTCTTAGGAGGGGGTGGATTTTTGAGCTAGGGTTTGGATAAGCTGCTTTTGAGAAGCCAGCA
GATTGGTGTCAATGGGGACAGGTTTTTTTTTTTTTTTTTTTTGAGACGGAGTCTTGCTCTCTCGCCAGGC
TGGAGTGCAGTGGCACAATCTCAGCTCACTGCAACCCTTGACTCCCTGGTTCAAGTGATTCTCCTGCCTC
AGCCTCCCGAGTAGGTGGGGTTACAGGCATGTGCTGCCATGCCCAGCTAATTTTTGTATTTTTAGTAGAG
ACGGGGTGGGGTTTCACCATGTTGGCCAGGATGGTCTTGATCTCCTGATCTCATGATCTGCCCACCTTGG
CCTCCCAAAGTGCTGGGATTACAGGTGTGAGCCACTGCGCCCGACCCCAGGGGACAGGTTTAGTCAGATT
CCAGGGTGGTCCTCATGGAGGGGACGCACAGGCAGGAGGGCAGGGAGAGAAAGCACAGAAAGCAGACCTG
CTAGGAGAGGACCAGGATAGGGAGGGGCAGGGACAAGGACATGGGACTTCTGGGAGCAGGTGGCCGGGGG
TCAACGTCCAGGAGGATTTCCTGAACCAGTTAGAGGACTACTGAACCCCCCTTTCACCTATCTATGCAGC
CCTTTACTTTTACAATATATACATTTCTATTTATTTTATTTATTTATGGTTTTTTTTTTTTTTTGAGACG
GAGTCTCACTCTGTCACCCAGGCTGGAGTGCAGAGGTGTGATCTCGGCTCACTTCAACCTCCGCCTCCTG
GGTTCAAGCAATCCTCCCACCTCAGCCTCCCAAGTAGCTGGGATTATAGGTGTCCGCCCCCACCTCTGGC
TTTTTTTTTTTTTTTTTTTTTTGAGACAGAGTCTCACTCTGTCACCCAGGCTGGAGTGCAGTGGCATGGT
CTTGGCTCACTGCAACCTCTGCCTCCCGGGTTCAAGTGATTCTCCTGCCTCAGCCTCCTGAGTAGCTGGG
ACTATAGGCACACGCCACCATGCCTGGCTAATTTTTGTAATTTTAGTGGAGACGGGGTTTCACCATGTTG
GCCAGGCTAGTCTCAAACTCCTGAGCTCAGATGATCCACCCACCTCAGCCTCCCAAAGTGCTGGGATTAC
AGGCATGAGCCACCACACTTGGCCACCTCTGGCTAATTTTTGTATTTTTAGTAGAGATGGGTTTTCACCA
TGTTGGCCAGGCTGGTCTCGAACTCCTGACCTCAAGTGATCTGCCCACCTCGGCCTCCCAAAGTGCTGGA
ATTACAGGCGTGAGCCATCATGCCGGGCCTACGTTAATTTTTGAGACAGAGTCTTGCTCTGTCGCCCAGG
CTGGAGTGCAGTGGCATGCTCTGATCGTAGCTCACTGCAGCCTTGGACTCTTGGGCTCAAGTGATCCTCC
CACCTCAGCTTCCCAAGTAGCTGGAATTACAGGCACGTGCCACCATGCCCTCTGTGGAGCCCTTTCTGAC
TTCCATCAACTCCCTGCGACCACCCCGCATCCTTCACCTCTCAGTCCGCACTCCCTCCCAGGCCAACTGC
ATGGTGAGTGTGTGTGTGTGCTCCCAGGCCAACTGCATGGTGAGTGTGTGTGTGTGCAAGTGTGCGTGTG
AATGTTACCATTTTTCTGACTTGCCATTCTTCAGTAGCAACTCTACAGATGCGACAGCTCTTCCCCGTTC
CTCTTTAGTCCATGAATGGTGGGCAGTATAAATATTATTTTCACTTGCAACTCAGATGGATTGAACAAAA
ACAGGAATAACAGCTCAGCCTCCTGTATATTTGACGCTAGATGCTGTTGGAAAGGCAGCTTTGTGGGGCA
AAAAAAAACAAAAATTGAGTGAGCTTGCTTATATATACCAAAGATGCTCTTAGACTTCAATGTTTCTCAA
AGGGTGAGATTGTGTGTTACCTCACCAGAACCACCTGGGCAGTGCATTAAAAAGGCACACTCCTGGAGCA
GAAGCTCTGGGACAGTACTTGTAGGTTTGATTTTCTAGCCAGCCCCCAGATGATTCTGGGGCTTACTACA
GCTTGAAAACCACCAAGCCTCAGATGATTAGAGGTAAACCAAATCTGGCACAGATGAGATTCCCACACAG
ATGTATTGGTAGATGCATTTATTGTATCGCCTGCATTGGCGGCGGGGGTCTCTCTACACATTTTGTTTAA
ACCTGGGCCAGGTTAATCACGTTGGGAATTTAGTGATGTACCCAGCAAGGGTGGTTTCCCAAGCTCTCCG
GGTAATTGATAGGGCTTGGGTTGCCAGTCTGATTGTGGTAACAAGCAAAGGAAGAAGTAGGGACTGTAAG
GCGGGCAGACCTAGGGAACAGTTTCCAAACAGGGTTGGGGGGGAAAGACCGGAAAGGACTTGGTACAGGA
ATTTAAATGTTTCCTTTCGGATGGAGATGGCAGGGGTCCTGCTGTTTTCAGCAATAGTTACCGCAGTGCC
TGTCTGCTGGGCTCCTCTGGAGTCTTCTCTTTGGACCCTGAAACTTGAGAGGGTCTGCATGCCTGAATCT
GAATGCGGCTCCTTTAGGCCCCTCCCTTTGACCCCTGGAGCCTGGAGAAGGGGTGGGGGAAGGGTGGGGA
AAACAGGCCTGGCTGTTAAGGTTTACCAGGTTCCTTTTGGCCACACCCCCCTTAGTATAAACTTAACACT
TCTCTCCAGGGTCCAGTTTCTAAAGCAAAGTGACAGCATCGACACGTCCCTAAAGCAAACTTGTCATGAA
GTCCCTCACCTCTCAAATGTGAGTCCCTCCTGGCTTCCTTTGATTTTATCTGTAATATTTAGCAGATACG
TGTGATTCTATTAGGCTAGTTAGGAAACCAAGGCCCAAAGAAGCTAGGGGTTATTTAAGGTCTTGTCATC
AGCTGTAAAACACCCTGCTCAGCGCCCTGCTCAGTGAAAGACCTGGCATCCCCTCCCAGTTGAATTTCCT
GGTAAGGGGCACCTGAGATCCGGTATCAGTGGGGTACTGCTACTCTCCCACCCCTGCCCTGGCATCTCTG
CTCCCTGGCCCCAGACTCCGTCACTGGTAGAGCTGGGCAGACCCAGCAGTTCCAGGATAGCGGGTGGAAG
TGAGCAGGAAGGAAGAGCTGGGTTTGTGGAGAAGCCTTCCCCTTATGGGGCAGGCTATAGGGAAAGGGAA
GGCAGCCACCAAAGCTCCAGAGCCTGATGGCGCATCTATTGGGCTGGGACTTCCCAGCATACACCATTAG
GGAAGTGTAGGGTTGGCTGGGAGAGAACGAGGGAGCCAGGGGCTCTGGGATAGAGGTGGGGAGAGAAGGG
GAGGTGAGGGTATCAGCAAGAGGGAAGTTGGGCTTTCCTTTCCAGCAGTTGGGCTCCCCCATCCTGCATC
TTCCTCCTTGATTTAGAAGAATTCATCTAGAGTGTCAGTGCAAAAAGCCCCTTACCACTCCCCCTCCCTA
ACAGATACCCTAAGGCTGTGCTTGTGCTTTTCATTCCTGCCATTCATCTGCCAGGAAGCTCATGCAGTTA
CAGATGCCCCCTAGATCTGGCCATTCCTGTCTGAAAAACCTGGGAGGTTGTGCCCATTCCTAGTTTCCGC
TCTTTCATCTTCCCTTGAGCCTTCCTGCCTTGACTGATCCCAGCTGCCTCTGAGTTCCTTCGGCACATGT
AGAAAGCATCCCTACCACATAATTTACCATCTAATTGCATGCTGTCTTGAATGAGGGCTGTGGTTTCATG
TGTGGTGGGTCTTGGGTACACAATTAGGCTGTGAGTAACAGAGGCCTAGAGCCCCATCATGTCTACTAGC
TCTTGCACCTCTTTGCTTCCCTTCCCCCCAGCCTACCTACTGCAGGTGCTAAGGACTGACTGACCTGATC
CGGTGTTTAGGTGAGCTTCAGGCAGGCTCCCAGGATAAAGCACAATTCGCTTACAGGTTCTAAAACCAGA
CTGCTCAGTGCAGGGGTTAGGGGGGTGCCCCTGCCCGACAGCATTTGCCCAAATGCCGCTCCTCTCCCCA
GACTCAGGGAGGAGGTTCTGGGGCAGGGCCGAGGTTTGGGGTGATGGCATAGAGCCCAACCAGGCAGCAC
ACTGCAGTTGCCATGGTGACCCTTTTGCTTTTCTTCAGGAGGATGGATTTCTCTTTTAGCCCAAATCATC
TGCCTGGGCTACTCTGAGAACCAGATTCCTAGCCCCCTACCTTCCACATTCTACCACAGGGGGCAGCTTG
TCAGAGGCTTTGGTTTGAAATCACAGTCCAGCATCAGACAGCAACAGGTTGTGCTTGATCAGGGAGCAGG
TGGGAAAGGCCCAGAGGAGGGCTCACCTGGGACGCATGCCCCGAGGGAGCTGACCTCCATCTCCTTCTGC
TTCCCCTCCTCTCTCCAGACCACTTCTCTCCAGTGAATTGCTCTGACCTTTCAGGTGGGCTCGGGCCTCT
CTAGGCCTCAGTGTTCTCATTTGTAACAGACACAGCCAGGGTCTCCGTGTTTCCACACCTGGCTAGTATT
AGAATTACCCAGGGAGGTTTTAATAGATTCCTGGGCCCCACCTTAAGCCCGCTGATCTACGTAGGTAGAC
CCCAAGAATCTGTATTTTTAAAGCCCTCCTTGGGTGATCCAGATGCAATTAAACCAAAGTGAATCTGGGG
TTCTCCTGGCGAGGTGATTAGCAAGGCCCTCGTGAAAAGAAAGGAAGTTCTGCCTGTGCTAAGGATTGAC
TGGCCTCTCCGGGCTTCCAGGAGCTTCTGGAGAGCCCCTTACAGCCTAGTCTTGTCAGGAAGCGGGTGTT
TCCTGGTCATGCCGGCTAAGCCAAGACCAGACCCCCACTTCTCTCCCTCCAGAGAAGCCAGGAGAGAGAG
AATGACAGGAAGGGGGGATGTTGCCATAACAACAAGCTCCAAGCAGAGGCCTCCCTTCCAGGGGCTGGTA
CTTCACCCCAGTCCTGGTGGTTTCCATGGAGATGGGTTACTGAGGGACAGGGGGAATGTCTGCCCCACTT
AGAGCATCAGCCAGGAGCTGCCAGAAGGCAGAACACCGCTAGGTGCGAGCCTTTGGACCCCTCTGCTCTC
CGGGTGCACATCTGACACCTTCCCCCTGCTTAGTTGCTCTCTCTGCGACTGGTCTCCTTAGCAACAAGTC
CTGCTAACTCCACGCTGTTGGCTTCATGGCTATTTTTAGCTTCACTGCCAGCAGCCCAGTCCTGGTCATT
TGCTTGCAACCTCCATATGGATTGCAAGGGAATAGGAAAACAAAGATTCCCAGCCTCTTTTGGTAGACTG
GGACCTCGTAGCAACGATTAGAATTCCTGCTATGAAATGGATCCATTCCCCAACTTGGAACTTGGGCAGT
GGATTTGGGATAAGGTTCCTGAGACAACACTGCATTCAGTTCATTTTTAATCCTCTACTATGGGCAAGAT
GCTCTAGCCCACACTGCTTCCATACGAAGGTGAATGAGGTCCAGTCCCTGCCCTCTAGGAATATTTAATC
TTGTAAAGGAGAAAGATGAGTACACAATCACCTTCGCACAAGGCAAAGACTGGCAGGTCCTAACAGAGGC
TTACAAAGTACAAGGGGAGTGTGGCAGGGATGGGGTAGAAGATAAGGGACAGAAGGGAAAGATAAGGGAC
AGAAGGGAAAGATAAGGGACAGAAGGGGAGGATAAGGGTGAGGGGCCAGGGCAGCACTGTCAAAAGAACT
TTCTGGGATGATGGCAATGTTTTAAAATCAGTGCCATCGAATTCAGTAGCCCCCAGCCACATGTGGCTCT
TGGACACTTGGAATGTAGCCAGTGTGACTGAGGAACTAAATTTTAATTTGTATTTAATTCAAATAGACAC
ATGCAGCCAGTAACTGCCATCCTGGACAGTGCAAGGCTAGAGGAACTTTCATGGTGGAGGTCACTGGCGG
ACTGGTGTAGAAGGATATGCAGGGTTTAGGTGGTGAAAGTTGAAGGGGAGGAACCTTCCAGGAAGAAGGA
ACAGCATGAGGAATAGCAAGTGGGCGGGCAAAGCGTGGGCTTCATGATATCACACGGAGGCCTCCAGATT
CGTGGGGGCCTAGAATGTCACGCAGAGTGTTGACTGATTGGCATTGAGGCTTTTGAGCTGTAGGTGGGAG
GAGGACGGGTTCAGAACAGACATCCATCCTGACCTCTCTTTTTGTCTGGATCAGGTCTCCCTGGTCTTGG
GGTTGGTGGGAATACGTGTGGGGTGGAAATACGTGTGCAGTGACTTCATATGTGTTGCTTTGCAGATGGC
TTTATAGGGACCATGGCTTCTCTGATTCAGAGTAGAGAGACTCGCATGGTAAATGTCCTGGTGGGATGAC
AGCAACATCATCTACCCTCATCCTGCCGGAACTGGATTTTATGACAGAGCATGCCACAGAGCACTGAACT
TTGAGTAAAACGTCAGGCATGAGCTCCGGGCGTTGCTGCTCTTTGGAGAGGTATCATGATGGGCTAGTGT
CTCGTCTCAATTCTTTGAGGCTGGAAAGTCACATTAACATACTTATGATCATGACTCTTCAATGACCCAG
GAGAATGATGCACCCCAGAATGTTAGATTTACCAATTGATAACAGGACATGTCCTTGCCACACAAATCAT
AATGCTTGTCCTGCTGTTTTCTGGCAACTTACTGGTATCAGAGTCATTGGAAAATGTAATTAAAATTGGA
AGTTTGCTTGATGAATACAGGATCAGGGCAGAGAGGTGTCCTTCAGAGGTATTTTTGTCGGGCTTAGGAG
GCCTATGGGATCACTGTTGTCTGCCCACCAAACAGGAACTGTGCTCTCATTCTCAGGTCACTCTCTCCAT
TGGACCACACTGGATACTTCCTTTCCAGCACCTCTTTTAGGCTCTTGACATCCAGCATATTTTCTTCTCA
TCCAGGAGTTCCTCCTTTCAACATACTTTTATAACAAATCCAAGAACATGGCTTATTTTACATGGTGCTG
CTATTTGAGTTTGGCTATCTTTAACTCAAACAAAAGACCTATCTGGTAAACTACCAGGGAGGTTTACTTT
GGAAGCTCAAGAGCATGTGTGACCTGAGGATCCTTGAAGATTGCCCCATTTTAGTAAGGGGATTTTTCCT
TTTACTAAAATCACCCCTGCTCTAGATCTTGGGGTCTCTTCTGCTTTGGGGTCTGACTGTGCATTGACCA
CAAAGAAGACACATCTCTGTTTAAGCGACTCAGTCTAGAAAAGGCAGTGTTTCCTGTCTGCATAGTTACA
GGAGAGGGAAGAAAGATGCTCTGGGTTGTTAAGGGGACACCTTTGAGCCTAGGCTTCCTTTTGCTTGTTG
GGAAGTTGTGTGGAAGAGGTAGGATTTGACTTTAATGTTAAAGGATGAGAATAAGGAAGTTTGACGGCCG
GAGGGAACAGCTACCTAATATGTGGCCAAGAGAAAGTGAACAGTTCTGGCCAGTTCTGACACCTTGAGTC
CTTGCTGCACGCACGGCATCATTGCAGTCTTATGTCCCAAGACCACAGAATGTGGAGTCTAAGGTGTTTA
GCCGCATTATAATGGAAGAGCGGGTATCGTACATGAGACAGTGTCTGCAAAAGGCCACCAAGCCAGAACC
AGAAACTTCTTTTGAGAGGAGGCCTGAAGTAAGTTCTCAGGCCCGTTGACCGGCATGGATGCAGAAGAGG
TATCAGTGAGAGAAGCAGCGAAGCAAGTCATGCTGCATTCCCTAAAGGTTGGGAGCAGGCAAGAGGGACA
AATTCTGGGCTTCTCACTTCTGCTTGGGGAGCCCCTGGAAATGTGTACCAGGCAAATACCTTAGTTGGGT
TGAAAGATGGGTTGGAACAGAAGAGGGCCTGTGTTTTTGTTTGTTTGCTTGTTTTTCGAATAGCTTCTTT
TCCTAGCTGTATGGATTCTGGGTCTCAGGGTTGGGGGGGAGTCTGTACCCAAGTCTTCTAGGTAGAAGGC
TCTCTGGTTTGGGGTCCTTGCTCTTCAGGGTTTGCTTGCTCTGATGGCCCTCAGGGGGAGGGGGGGCTCC
CTCACTGCACATCTCGTGACTTCATTATACCAGCACCTGTATGTCCCAGGTGACTGTGGCTGGGAGCCAG
GCACCTAGAGAATTGTCTCATTGTCATTAGGAGATGGTGGCGTTCCATGGCCAAAGAGGGCTGATGTCAT
CACTCGTTTTGCAGATGAGACAACAGATTTCTTGGGGGTTAAGTGACTTGTTTAAGGTCATGGTGGTGGA
AACAGAACTGAAGTCCAGATCTTTTTTTTTTTTTTTTTTGAGACGGAGTCTCGCTCTGTTGCCCAGGCTG
GAGTGCAGTGGCATGATCTCGGCTCACTGCAACATCCGCCTCCTAAGTTGAAGCGATTCTCTTGCCTCAG
CCTCCCAAGTAGCTGGGATTACTGGCGCACGCCACCACGCCTGGCTAATTTTTGTATTTTTAGTAGAGAC
AAGGTTTCACCATGTTAGTCAGGCCGGTCTCAAACTCCTGACCTCATGATCCGCCTGCCTCAGCCTTCCA
AAGTGCTGGGATTATAGGCGTGAGCCACCGCGCTCGGCCAAGTCCAGATCTTCTAACAAGTGCCGCTGCC
CAAATAGCCCTCTGCTGTGGGGTGCATTTTCCTCCATTTCCTCAGTTCTTCCTTCTAATTCATCTTGCCA
ACGGCAACTAGGCTGATTTTTCCAAAATACTCATTCATCTTGTCAGAAAACCTGCGGTTATTCTTCCCTG
CTACAGAATATACCCAAGGACGCACCTGAAGGCTTGCCATTACCTTGCCCTGTCGTGTACTGGGAGGGTG
GAGGTGGGCGAGGGTCTCCTCCCTCCCCAGCCCGGCAGCTCTTGCTCATCCTACCCATCTCACCTCATTC
CAAGTCCGATCCAGCCTCCAGGCCCAGTCGGCTCACCTGGAACTGACCTCTGACCTCTTTTGTCATCCAT
GCCGCCCATTTTTTTCTACTTGGTATTTGTGGCATAGTTACCTTTACATATGTTTGTTTTACAGTGATCC
TTTCATATTTCTCCAAGTCTAGTGGAATCTTCAACCCCTCGAGGGCAGAGCCAACAGGGTCTATTTCTTT
ATCTGATCCTACAGCCAACGTAATGGAGGGCTGTGGGTGGGGACTGCGTCTGCCTTGGGGGTAGGTGCCT
TTGTTCAGGAGGAGGAAGCTTGAAATGGCGGAGGCTGCACCTGGAGGCCGCACCTGGAGGCCCCAGGAGA
GGAGTCAGGTCTTCTCGATCTGCAGATGTTTGAGCCTGGGAATGAAGGAATTGCTGAACTTTCTGAAGGA
GCGCCCTCGCCGCGACCAACCTTGCAAACAGGAAAATGAGAAATCCAGGGAAGGCCCAGAGTGACGCAGG
GGCCCTGGGACTCGAAGCCTGACCTCCTCACGCCGCGCTTTTTGAGGCCCCCCCGCTTCTCTATTCACCT
GTAGTGTGGAGGCGGGAGACCCCCCAAACAATCCCCGATCTGGAGCGCTCCCAATGCCTGCGCGCGCCTG
CTGTCACTCTCCGTCTGTGTGCTGAGTTTTCCTACAGCTTCCTGGGCCTCCTATCTGTAAGCTTTTTCTT
TTTTTTTTTTTGGTTGTGCTTCAGAGAAACTCACTTTTCACAACTTTCTCCCGGCTCTCCCAGGCCGTCC
GAAAGCTCCGGCTTGCTTTCGCCCGGACCCCCGGCTCCCTCCGGGCAGGCGGCTCGGGAGCAGCCCCTTC
CCTCCCCTCCCGGCCCCCCGGCCCCGCGCTAATCTCTTCCAGAGCTGGGGGAGGGGCCAGGCGGTCTTCC
CGAAGGCGGGGCGCTCCCTGCAGCCCGGCCTGGGCGGGCCCTGGGAACGGGGGGGGAACGGCCTCGCCCC
CCGGCCCCGCGCCCCTCGGACCGGAGAAGAGGGGCTGGCCCAGCGCCAGCGTCGGAGCGCCGGCCCCCTC
CCCGGGCCGCTCGCAGCCAACCAGGCCCTCCAGCGGGGCCCACGTGACCTGGAGTCCTAGACAAAGAAAA
TGTTCCCTCCCTCCCCCCCGCCGCCCCCCTCCCCTCCCAGTGGCCCCCTCCGCCCCCAGCCCCATCGCCC
CCTTCCCCTCCCCCAAGACGGGCAGCTACTTCCAGAGCTTCAGGGCCGCGGCTCACACCTGAGCGCGACT
GCAGAGGGGCTGCACCTGGCCTTATGGGTAGGTTCCTGGACGGAGCCCCGGGGAGACCGGGGGCGGGGAG
GCGGCAGGGCCGGTGGGAGGATCTCCGCAGCCCTCCGGGTTTGAAAAGAGTGAAGTATGCCTCCCCGGCC
CGCAGCAGCCGCGGCGTCCCGGCCTGGCCCGAGTTCTGGAGATGCTGACTTAGTGCTGGCCCGCCGCCTT
CCTGCCCACGAGCCCCAAGGCCCCACTCCTCGAGGGCTCCAGTGGCACCTGGCGCCCTCGGGGGACAGGA
AGAGCAGCCGGCCGGGAGCCCGGGCAGTGGTAACTGCAGGCTGGGGCACGCGGAGCTGGTGGCCCAGGAG
TTGGGTTGGAGGCAGCTTCAGCTGAGCTGGCAGATGGGTGCCTGGCACTTCCCTGGTGACCTGCGTTTCT
GGCTGGGGGACCACGCGGTCCTCAGGGCCCTGCCAGCTTGCGGCCTCTTAAGAGGGACAGCTTCCTCTCC
TGCGAGTTCCTGGAAGCCTCCAGGCAGGCCGCCAGAAAGAAGTGGTGGGGGGCGGGAGAGCACGGTGCGA
CCCTGACAGGGGGCCGGCCTGTGACACCAGGGTAGCCCAAGGCCGCATGGGCTTGGGGGCGGTGGCGCTC
AGACCTCTGACCTTTGGAATGGGAAGCGCTGCAGTCGCAGGGGGAGGTGGAGGCGGGACAACTGCCTGAG
GAGATGGGACTGGACCGGAGGCAGTAGTCACCGGAGTTCCCGGTGGCTTTCCCAGTCTGGACGGACTCCA
GGCCCAGGGGCTTCCGTGAGCCAGCAGGGAGTGGGGAATAGTGTGGTGTCTCTTGAATGCCGCCTGGTTT
CTCCGCCCCCCTGGGGGTGAGCAGCCCGCAGCCCAAATGCGAGAGCCTCGAGTTCACCCCTGTTTCTCTT
ATTGTTTGCCTGGGGAGGAAGGCAGGAGTGCAGAGGGAAAGGGGTGCTTTCCCTACGCCCCAGACAGGCC
TAGTCTAGTGAGGCCCCTCCCCCACCCAGCTCCGGCTGCCCCTTCCCCTTTCGTCAGGCCCCCTGCTGCT
CACCCACCCCCCCCCCCGCTTCTTCCTTCCCCCACCCCACTGCCCCAGTCCTGATGGAGGCCCCTTCCTC
CTCCGCCAGCCCTTACCGGGCCGGGTGCGTGTTCTGCACCAAGTGGGCTGGGGATGCTGAACAGCTCGCA
GCGCGGCATCTGACAAGCTGGGGGAAGTTGTGGGGGAAGAAGAATGTTCAGGAACTCGCTGTGAAGGGGC
TGAGAGACAAAGGGGCCGTGGGGGAGTGCTGAGGGTGGTGTCAGGCACGACTCCTAGACCTGGGGAGCCA
GAGGAGGAGCCCAGCCCACCTGCTGCTGCCGCTGCCAGGAACTGATAGCGGGTCCTTTCCCCTTCCCGCC
CACTGCTACCTGCACACCTCGTACTGGCTCAGGGGCCTCGCCACGCCTGGAAACGGGGTCTTTGGATCAT
CCGAGGCATCACCCTAGGTCAGCCACCCATGCCTGAAGCTTGAGAAGGCGTGGTGGGCGAGGAGCAGCGG
CGTGGTGTGGGCTCCTCTGCGAGGGTAGCTGACGCAGCTGAGGATCCCAGCAAGGGAGGCACCCTCCCCC
ATAGCGGGGTCCACACTTCAGTGCACCTTCTCGTAGGGGAGGAGGCGATGGCTAATTGATAGTAGGAAGG
GGAGGGCAGAGTTCTGGCCAGGTGAGAGTGGGGCTAGGGAGCGATGAGCAATTACTCCACATTCTTCCAC
CCCCGCAGTCCGATCCGTGAGCTCATTCCGCCATCCATGAGCTCATTTTCCCAGCTGTTTGGGGGTTGGG
GGCAAAGGATGCATTATCCTGTTTTAGAATTTGTTGAGTCAGAGGAAGAACTAGGACTTAACATCTTCAG
TGTTTTTCGGAATCGCCATAAGAAACAGATTGGTCTGTGGCTTGGAGGCAAAGACTTGAAGACTCAGTGA
AATTAAATGAATCTGTAAGACAATACATTGGACCACTGAGCTTCAAACAAAGGTTCTTAACCTGGGACCC
TTGGATCTGGGGGGTCCATTGACAGGCTTTGGGAGAGTCCTGGGAATCATTGGTCCATATATACACCAGA
TTTTTAAACAGGTCAGAAAAGCTTTCCAGAAATCCTTCCGTTCAGGTGCCTTTTTATGTTGGAGCAACTC
CTAGAAAGGACTGCTCCAAAGTCAGCTGCTGGTTGTGAGGCTCCGACAGGAATGTAGAGGCTGCAACTTC
AGATTAGAGCTCTTCTTCATACTCCAGGTTGCTTGCTCATCCTCTTTACCCTTGAGGATGGGTTTGCATT
CGCAGCTCTAGGCCCATGGGGGAGCTCTTTTGAAAGGCATGTAGAAAATGGGGTTTTCTCCCCTCTAAAT
CTGGTATGGTGGAAATGGGTGCAAAGCCTTGAAGTCAGAGGGCCGTGTTTCCTAGATTCAGTAAAAGGCT
AATGGTTCTCAGAGCTAAGTATCAAGGATTTCATTCTCCTTTGTAGGGATCCTGGAGCGGGTTGTGAGAA
GGAATGGGCGCGTGGATCGTAGCCTGAAAGACGAGTGTGATACGGTGAAAGGATGGAGGCTGTGCAATGG
GAGAATAACTGGGGTTTGGCTTATTAATTTGTTGGTGGGATGGCAGAGAGCACGCCTGGTCTATAGTGCC
TGCTCAGTAAATAACTGAATGAATACATGTATTTATGGATCTGAAGGGTGCACATAGAAGGGGTCGAATC
TGGTAACAATACACACTGGGTGTGATCCATAAGAACTCCAGCATGGCGTCTGAGCATGGGAACAGACAGG
CTCTTGGGGAGCTTCCTATGGTGTTTGAGCATGTGCTGGGGGGAGATGGGCTCTTTAAACCCAGAGCAGT
GATTTCTTCGTCAGCAGACATCACAAAACTAGAATTGTGGCCTGGAAACCAGGAATAGGAAGGATTGATT
GCCCAGGGGAATTGTGGAGAAGAAGAGAGGGTCCCCCTTGCTTTTTAAATTTTTGGTGACAGGGTCTTGC
TATGTTGCCCAGGCTGGTCTTGAACTCCTGGGCTCAAGTGATCCTCCCTGCTCAGTAACTGGGATTACAG
GCACATGCCTGGCTGAAGATCCTAGTCTTGATTAAGAAGATAGGAGAGGGTGGAACCAATATCTGTGTCT
AAACAATAGTGCCTGATTAATATTTGGATAGAATTAACATCTCCATAGGTAGGGGGAGGGATAGCATTAG
GAGATATACGGTATAATGCTAAATGACAAGTTAATGGGTGCAGCACACCAACATGGCACATGAATACATA
TGTAACAAACCTGCACGTTGTGCACATGTACCCTAAAACTTAAAGTATAATAATAATAAAATTAAAAAAA
AAACATTAACGTAGGCTAGACACAGTGGCTCATGCCTGTAATCCCAGCACTTTGGGAGGCTGAGGCGGGC
AAATCACCTGAGGTCAGGAGTTAGAGACCAGCCTGGTCAATATGGCGAAACCCCATCTCTACTAAAAATA
CAAAAATTAGCCAGGTGTGGTGGTACATGCCTGTAATCCCAGCTACTCGGGAGGCTGAGACAGGCGAATC
GCTTGAACCTGGGAGGTGGAGGTTGCAGTGAGCCGAGATTGCGCCACTGCACTCCAGCCTGGGTGAAAGA
GTGACTCTGTCTCCAAAAAAAAAAATAAATAAATAACATTTCCGTATCATAGGGTAGGCAACCGATATGC
TGATGAGATTGAGGAAGATTAGATCTGGGAGGAAGGTTTCCTAAAAGGCAGAAAAGAAGGCTGGAATTGT
GTGACTTGAGTTCACCTCTCTACTTCAAGTCTTTCCATTTTTCACGGCAAGGCAGCTGGTTGGCTCTCTC
CAGCCCTTGACCTTCTGTCACTGTTCCGGGTTTTGACAAACATGGTCCCCTTGAGTGTCAGGTGGCCTTG
CTAATTCCTGGAGAGGTCAAGGTGACTTTTTGTTTTTTGTTTCCCCAGGCTGAGAAGAAAGCCAAGGTCA
TTGCAGGAATGAATGCTGTGGAAGAAAACCAGGGGCCCGGGGAGTCTCAGAAGGTGGAGGAGGCCAGCCC
TCCTGCTGTGCAGCAGCCCACTGACCCCGCATCCCCCACTGTGGCTACCACGCCTGAGCCCGTGGGGTCC
GATGCTGGGGACAAGAATGCCACCAAAGCAGGCGATGACGAGCCAGAGTACGAGGTGAGCGGCTTCTTCA
CCAGCCGTGGCCAGCTCTTTCCGGGGGCCTTCCACTGCCAGCTCCTCCCGTCCTGCTCTCCTCTCTCCAT
CTCCCACGGGCCGCTCGCTCTCTCCACCTGACCCCGGGCCTCTTCGTCTCCCCGATTTTGCTCTGTCTTG
CCTCATTCAGATGGAGCTCCCTCCTGGCTGTCCTGCCCCCCAGATGCCCAGGGCTGGGGTTGCCATGGGG
GGATCAGGGTGGCAGGGCCTCGTGACCACTGTGTAATGATTTCTGCTCCTTGGGGCTCCAGGACGGCCGG
GGCTTTGGCATTGGGGAGCTGGTGTGGGGGAAACTGCGGGGCTTCTCCTGGTGGCCAGGCCGCATTGTGT
CTTGGTGGATGACGGGCCGGAGCCGAGCAGCTGAAGGCACCCGCTGGGTCATGTGGTTCGGAGACGGCAA
ATTCTCAGTGGTAAGTTGTGGGGTTTGGCAGTAGCCTGGGGTGGGGGAAGGTTCTTGATCCCAGGGCCTG
TGGTTTTGGCTGGGGGCAGTCTGGAATTGGGGTGATGGAAGTGGGTTCTGCTCACCCCTACTCTGCCTGT
GGTGGGTGGAAGAAGCTGTAGGGATAATTAGGTTGCACCTGTGGCTCAGAGAGCTAGGATGCTGGCCCAG
TTTTAGCCTTCCAGGAACCTATTCACTAGAATTCCTTTCCACCTGCACTCAGGTACTTCCCTACTCTTGG
TCTGGACTCCTCTTTGCATCGGGTAATTATTTATCACTGTATCTGGTCCCCTCCAGGGCTGAGACTGACT
CTCGAGGCTCCCAGCCCCTGCCAGTTGCAAGGCATGGGGTGGGTGCTTGCAAGTGTAAGCCTCGGCAAAC
AAGGCCTGGCTTGTCCCCCCAGGTGTGTGTTGAGAAGCTGATGCCGCTGAGCTCGTTTTGCAGTGCGTTC
CACCAGGCCACGTACAACAAGCAGCCCATGTACCGCAAAGCCATCTACGAGGTCCTGCAGGTGAGTGTCC
CTGCTGGGAGCTCGGAGGAGGAGCCTAGAGCACTAGGAGGCCTGGAAGTTGGAGTGCAGGTCGGGCTTGA
GCAGAACGGAGGCCCAGGAGGCAGCGACTCCTTTCTCTCCACCTCGCTCGCCCGTATGCACATCCCGCTC
CTCCTCAGCGGTTCCTCCCCATCTTGCCTGTGCCACCCTCACTACTCAGAGTCTGGCCTTGAGCCTGACC
CATCTGCCTTCCAGCCTGTCCTGACAACCCCAACCCTGGCGTGTCACCCTCCAGGTGGCCAGCAGCCGCG
CGGGGAAGCTGTTCCCGGTGTGCCACGACAGCGATGAGAGTGACACTGCCAAGGCCGTGGAGGTGCAGAA
CAAGCCCATGATTGAATGGGCCCTGGGGGGCTTCCAGCCTTCTGGCCCTAAGGGCCTGGAGCCACCAGAA
GGTAAATGAGGGCACCCAGCTTTCTGGGACCCCTGCCCGCCAGGCAGATCCACACCAGGGCTGGGAAAGC
CATGCTTAGGGAGGGCTGCCAAGGCCTCCACAGAGGGGCCCAGTGGGAACAGCCGGGGTCTCTCTTGCCC
TGGGGTCAGGACTTGAATGACCTGCTCTCTGTTGGTTGGCTAGAACCGTAGAGTTGGGAGGTGGGAACAA
GTTGGAGACCAGGCCGCACCCCACTGTAAGGAGGGTGGGGGAAGGGGCTGGAGTTTCCTGTCAGCCTGTA
ACTGACCTTGGCACCTGCTTTCCTCCTCCAGAAGAGAAGAATCCCTACAAAGAAGTGTACACGGACATGT
GGGTGGAACCTGAGGCAGCTGCCTACGCACCACCTCCACCAGCCAAAAAGCCCCGGAAGAGCACAGCGGA
GAAGCCCAAGGTCAAGGAGATTATTGATGAGCGCACAAGAGGTAGTTGGCCTGCTTCTGGAGAGGGTGGC
ACCAGGAGGCCTGCATCCGAGGAGCAGGCGCGGCCTCCTCTGACGCCAGCTCTCCTCCCCTTGCAGAGCG
GCTGGTGTACGAGGTGCGGCAGAAGTGCCGGAACATTGAGGGTAAGTTTGTTGCCTGGGAACTCTGGCAC
TCCTAGTGTGGCATGGGGCAGAGTAGCAGAGGCACTTAGGAAGGTTTGAATGACATGGGACCAGGGGCCA
CGTCCTGCGTGTCACACCACCTTCACCGTGCTTCGTGCACTGTCTGTCTTTCTGGTGCCATGATTCTGCT
CTCCTGTCAATGGCGTGGGCTGGTCTTCAGGGTGTGGGACTCCCCAGTGGCCACAGAAGGGCAGCCCCTC
TTGGGTGTGACTGAACTCACCTACAGGTCCCCTTTCAGCCTTTCACCTTTGCTCACCTCTCTCATCCTGG
GGCTGTCGCCCTCAGCCTGGTGCTGGTGGATCTGGCTCAGGACCCTCGGCCTGGGAGGATTGGGGCAGGG
GTCAGGACTTGAGGATTACCCCGGGAATCCCTCCTGAGTAACCTGGAACCAGGGCTGCTTCTCATGAGCA
GGTTTCTGTCCTGCTGTCACTCCATCCCTGTTTGTGGATGGTGGCATTGGTGCTCTGAGATGATGGCGTT
CGAGACTGGGGTCACAGTGCCTCCCTTTTCCGTGGGGCACCCTGGACTCTTTTCTGGCTGCCGCTCTGGC
ACACCCCTTTTTTGTGGCTGGTCTGGTGCTGGGCTCCGGCCCGGGGAGGGAGAGGCCCTTCGGTGGTACT
CACCCCATCCCCTCCCTCTGCTTTCCAGACATCTGCATCTCCTGTGGGAGCCTCAATGTTACCCTGGAAC
ACCCCCTCTTCGTTGGAGGAATGTGCCAAAACTGCAAGGTAGGAGCACACCCACCCAGGAGAGGTGCCGT
TGAGGCCGGCCCTTCGGCCTTCTGGCTGACTGTGTCCACCGCTTCTGGGCATGTACAGGGTGACACCCGG
TGTGCGTGTGATGTAGACTTCAGAGCTTCCTCTGGAAGCAGAGTCCTGATGACACGGGCCCTTGGGTTTC
AAGCTTTTGACTTGCTCTCATTTTTCTCCTCTGAAAGATTCTTTACCTCTCTCAAGCCCCATTTTAGTCC
CCAGATGTGGGGATGGGAGCAGAGCAGCGGGGACAAGCTCAGGCAGAGTGTAGCAGGGTTGCCGTGCCCT
GGGGAGCCCAGGAGAAAAACCCAGGGCTGCAGGAAGGCCCAAGGTGGTGGTGCAGCCCCCTCCCTGGCCT
TGTTCTCAGGCTGCTCTTTGGTTCTGTCCATGGCGGTGCACAGGCAGATGAGGTTTCCCCTTCCCAGATC
TGCAGCTGGGGCCTGACCCTGGCTAAGGTGGTGGCGGGGTCATGTCTTCAGGGCTTAGGCTCTGTGAGGC
CAGGTGTGGAGCCTCCCTTCGTCCTGGCCGTCCTGGGACCCGTCCTGGTGGTTTCTGACCCTTCCCGCTG
CTGTCTAGAACTGCTTTCTGGAGTGTGCGTACCAGTACGACGACGACGGCTACCAGTCCTACTGCACCAT
CTGCTGTGGGGGCCGTGAGGTGCTCATGTGCGGAAACAACAACTGCTGCAGGTGAGGCTGTTGTGGCCTC
CAGTGGTCTCCTTAGCTGGGCCCCAGGCTCCCGCCGCCCACCGCCTCCCCTCCTCTCCCTTCCCCACAGA
CCCTCCACCCCCTAGCCATGCTCCAGACCCGGTCTTTCCATTCCAGGTAGCACACCTTGGCCTCCCCGGA
CCAGGGCTGAGAGTCTCCTCTGCTCACTGGGTCTCCTTCCAGGTGCTTTTGCGTGGAGTGTGTGGACCTC
TTGGTGGGGCCGGGGGCTGCCCAGGCAGCCATTAAGGAAGACCCCTGGAACTGCTACATGTGCGGGCACA
AGGGTACCTACGGGCTGCTGCGGCGGCGAGAGGACTGGCCCTCCCGGCTCCAGATGTTCTTCGCTAATAA
CCACGACCAGGAATTTGTGAGTGCTGGGCCTGGGGCGCGGTCTCGAGCTCCCTTGGCTGAGCCTTGGTTG
TGGGGCCTGAGCTGTGCGCAGGGTGTGTGGGTCTAGGAGCCTGGCTGGAGGGCCAGCGCTGGGTGGGAGC
TTGGGACACCGCTGGGCCTGCATCTGACCTGTTGTGCTCACTGCTTAGGACCCTCCAAAGGTTTACCCAC
CTGTCCCAGCTGAGAAGAGGAAGCCCATCCGGGTGCTGTCTCTCTTTGATGGAATCGCTACAGGTGAGGG
GTGCAGGCCCAAGAGGTGCTGGCCGCCTTGTCCCAGGATGGGGGAGCCAGGTGTGTGGGCAACTCTGGCA
AAACGAAATGATGGTTAGCTTCACAGCTGTTAAAATATTATCCCAAAGGGGAAGCTTGTGTGTGAAGTGG
AAACGTATGCAGTCCGTGGACTTAGATTTGACCAAAAATAAAAAAGTAAAACAGGTGGTAAAGCAGCCTG
CCTGCTGGGCGCTTCCTTAGTAAGGAAGATTCAGCCACAGAGGCCGAGTGATCATTAGTCAGATTCACAG
AGATTTCACAGAGCCTCAAAGGACAGATTCAGCAAGACAGAAACATACACAGTTCTGTTGTGTTTTGATT
ATACACAAGATAGTCTAAAGAAGACTTATTTAAATCACCCATTATCCTTTTATTAATATTTTGGCATATG
TCCTTTTCAGATTTTTTTCTGTGCGTTTGTGTTTATATAACCATACATCCATAGATAGATAAATTAAGTG
GGTAGTGAATGCACACGGTTTTGTAACTTGCCTTGCATTTATTATATTATGATTGCTTTTTCATGTCAAG
CGATATAAAGCTACATTTTTTTTTTTTTTTTTGAGACGGAGTCTGTTGCCCAGGCTGGAGTGCAGTGGTG
CCATCTTGGCCCACTGCAACCTCTGCCTCCTGGGTTCAAGCAGTTCTCCTGCCTCAGCCTCCCGAGTAGC
TGGGACTGCAGGCACCCACCACCACGCCCAGCTAATTTTTGTGTTTTTAGTAGAGACGGTGTTTCACCGT
GTTGGCCAGGCTGGTCTCGATCTCTTGACCTTGTGATCCGCCCACCTCGGCCTCCCAAAGTGCTGGGATT
ACAGGCATGAGCCACCAGACCCGGCCAAAGCTACATTCTTTATTTGTAAGTGTTGCGTAAGGTTCCATTG
AGTCAAAGTGCCGTAATTTATTTAACGAACCCCTATTAGGTCATTTACTATTTTTTTTATTATTATTATA
AGTAGTGCTGTGACAAGCATCCTGATAGAATATATACTTGAACCATTCCCTCCTTAGGAAAACCTTTAGG
AGATGCTGGGTCACCCTGCAGGCATTGTTTGCTTCATGTGACTCTGGACACTGCAGAAAGCGTCCTAGGA
GATTGTGAAGACACATCCACCCCTCTGCCCTGCCCCCCCAGCCAAGGGCTTCCCGGCTTTTAAGAAAGGA
ATAGCTCTTCTTAGAATTCATGTCTTTGGCCAGAGGGAGATAGGCAGTCTCAACCCAGGGACCCTGGTTA
GAGGGTGAGGCGGAGGGGATGGCTTCCCCGGCCAGATGTTTGTCTTCTGTTCTCAGCTCCATGCTGCTCT
TCTGTAAACCTAGAGAAGTTCATTTACAGCTTTTTGTATCCCAGACTGGAACCTTGAGGCCTTCTTAAAT
CATGTCACACCCCAGGAGGCTGTAAAAGCTCCTTGGCTGGGGGACATGTTGGGAGCAGCAGAAACAGTGC
CGTACCCTGTTTCTCCCGAGACCCTCTCGGGATCTGCTCTGATTGTTTCCTTGCCTCAGTTCGGCTAGCA
TTTCTGGAGGGGGAGGGATCGTGGTAAGGAAATCTGTGTGGTTAGCTCGGGGGAAGCACAATGAAGGAGG
CCTCCTCTGTCTGGAAGAGCCTTTGCAATCCACGAGAGGGCTGAGTGAGGAATGGAAACTGCTCTGATGG
GGAGGCAAGGGATGCACAGAGCTGGGGACTCATAGCGGAGGGGGCGGTGATTCCAGGGGCTACTGGGTGG
GGCAGGGCATTTATGAAACCTTTGTGGAGCAGGGAACATTCAGGTTGAGTCAAAGGGTGGGGTAGAATTG
TAGCAGGAGGTGATAAGAGGGTATGGAAGGACCAAGTTGTAGACGGACAACAACAAAGAAAAGGCCCCCA
AAAAGTCAGCTGTGATTCTTCCTGGATCCTGGAACCATGTACTCTCCGGTCAGTTTTCTGCAGCTTTGAC
TGCCTTCCAGATAGGCCAAACGAGGCATGAGACAGAGCCTCGATGTCCTTACTATGGATACTCCAGTTGG
ATCCAGAAAGGATTTAAGGCATCTTCAGGGAAAAGATAGGACTTGGGCCTACAGCTGACCCCATGGGTCC
TGTTGGCCAGCAGGCTCACCTGCCGAGACCAGGGTGCCAGGGAGATGGCTCCAAGTAACGGTGCTGTCTG
CTGGCTGGTGCAGGGCTCCTGGTGCTGAAGGACTTGGGCATTCAGGTGGACCGCTACATTGCCTCGGAGG
TGTGTGAGGACTCCATCACGGTGGGCATGGTGCGGCACCAGGGGAAGATCATGTACGTCGGGGACGTCCG
CAGCGTCACACAGAAGCATGTATGTCCATGCTGTGGGGCGCAGCCCGTCTTCCCCTCCCTGCACACTCAG
CACCTGGAGGCAGCCCTTGCCTCCTTTCATTTTGTTCATTTGCCCTCTTTCTCTTCACCCTTTGCCCCAG
CCTCTTTTTCTCCTTATCTTCCATTTGTCCTCTGTCTTGAGAGGGTAAAGGGCAAATTGAAATGGGAATT
TGGGCTAGGCATGGTGGCTCATGCCTATAATGCCAGCACTTTGGGAGGCCAAGGTAGGAGAATCCCTTGA
GGCCAGGGGTTTAAGACTAGCTTGGGCAACATAGTGCCTACTTGAGCTCTGGCTACTCCCTGGGCTGGTT
TCTGGACAGTTTCTAGTAGAGCTCTTTGGATTAGAGAGAGGTTCCTGTCCTCCCAATACTTTTGGCAAAT
AGCTTTATCAGCTTCCATATCTGGGAGGCTCAGGCAGGGTCTTCCCTTAAGCATGACTACAGGCCCTCTG
GAGTCCTGAAGGGCAACTGGAGGACATTCACATGGCCCAGACACCCTGCCTAAGACACTCAGCATGCCAG
GTACCTTCAGACTCAGCCGTCCACTTGGGTCACCTTCCCAGTGGGTGGCTGTGTTGCCTGCCTGGGTGAT
ACTAGTTTCCCCATTGGCCTAAGAAATGATCCAACTTGGTCCCGTTCTTGTTTAGGACTTGTCCATGGTT
GCAGCTAGGGTCGTGAGCCTGCAGAGCAGCTGGGTCTCCTCTCTTTCGTGTCAAAGGACTTCTTTGCCAA
GTTCACAGATAATTTCTCTTTCTTCCTGTCTGCCTCTGTCCCTGGACAGCAGGCCCATCACGTTGCCTTT
ATCCTCCCAGATCCAGGAGTGGGGCCCATTCGATCTGGTGATTGGGGGCAGTCCCTGCAATGACCTCTCC
ATCGTCAACCCTGCTCGCAAGGGCCTCTACGGTAGGTACCATCCTGTCCCCTCCACCTTCTCAGCTGGTG
CTTCCGCACATAGGCTTCCTCCTTGGATATTTCTGCCCTGGGACAGCTATTCCCGATGACCCTGTCTTCC
CGTGCCCTCCCCGTGCCCGAGCCACACCACTGTCCTATGCAGACAGCCCCAGCTGATGGCTTTCTCTTCC
GACCTCTCAGAGGGCACTGGCCGGCTCTTCTTTGAGTTCTACCGCCTCCTGCATGATGCGCGGCCCAAGG
AGGGAGATGATCGCCCCTTCTTCTGGCTCTTTGAGAATGTGGTGGCCATGGGCGTTAGTGACAAGAGGGA
CATCTCGCGATTTCTCGAGGTATAGCCAGCAACCTTGGTTTGGCCAGCTCACTAATGGCTTCTACCTTGG
ACTGCTGCTTCATCCTGTCTCATCTGCATTGTGGAGCTGGGGACTGGTGACTTCTGCTTTGCAAGGGGCC
TGTTTGGGAAGCCCCAGGCTTCCTGGATAGAAAGGGAGGAAGCAGGGTTAGAGGCCTACCTCGGAAAGAA
CCAGTGGTCTCACTCTAAGAGGTGGCATTGTGGGTGTGGACCTCCGGGGTTGAAGGACCTGTCACTCAAT
CAGGTGGGGTGCCAGACTCCTGGGGTTGGGTATTCTGTATGGTGTTTGTTACACACCAGGATCTTTGAGA
GGACAAAATCAGTTTTCCCTGAGCAAGAAGGGCCTATCCTCACTCCCTGGTACCATGACAGGCACCTGAG
AAGAAAACAAGACTTAGCTCAGTTTTCATTCCTTATCATGTGAGTCCCCGTTTAACCCTGAGCCAAGTCT
GAGCCGCGTGCAGAGGGCTTCTGCTGCCAGAGGCGTGCTTGGAGTGGCCCTTCTGGGAGCTTGAGCCCAC
CTGTGCTCCTGAGTCTTGGGGTGGGTCACACTGTGAGCTGGTGTTCTGACTGTAAGTGTGAGCAGGCGTC
TGTTTGAGTTCTAATCAGGGCTCATGGTAAACACACCCATACTCGCTGGAGCCTGGGCTCTCCCAGCCTC
TGGGTAGTCTAGAGCAGCACTGTGCAATATGGTAGCCACCAGCCACATGCGGCTGTTTACATTAACATTA
ACCATTCAGCTCCTCAATCACATTGGCCTCATTTTAGGTACAAAATAGCCACCTGTACTAGTGGGCTAGT
GGCCACTGAATTGAACAGTGTAGAGGACAACCCACCATCGCAGAAGTTCATTTGGACAATGCTGATCTAG
AACGTTCCAGTGACAGCTTGTGGAATGTGGCTAGGTGTAATTCCAGCTTCTCCTGGAGGCTGCAGGCTAG
CCCAGTGTGTGGCTCCTGAGAGAGAAGTAAGAGGCTTTGAGGCTTTAAGGCTCGACCCCAGCAGCATGCC
GGCGCTGTTTCATGCTCCTCCTTGGCTCATCTTCAAACCGTCTCCTGTTTTGTAGTCCAACCCTGTGATG
ATTGATGCCAAAGAAGTGTCAGCTGCACACAGGGCCCGCTACTTCTGGGGTAACCTTCCCGGTATGAACA
GGTTGGTGAAAGCTCCTGGGCCTGGGGGGCTGTGGGCTGGGACTGCAGGTGGGATGACCCATAGTGGGGA
AGCCCCTGAGCCGGGCCCTCTCTGGCCGGCCTCTGCTGAATGACTAGCTGCTCTGCCATGTTCCTTATTT
CCTTTTCCTTGAGGAGGAAGAGGAGTCAGGGTATAGAAAATACTCGTCTGAAACCAACAACTAGTGGTCT
TAAAGCAGGACCTTTAAAAGTACTCTCTTTATACCAAAGAAGAGTCCTGTGCTGCTGAGTTAGGTAGTTG
GTTTTCTGACATTGGTGATGTGTCCCGAGCCCTCTGGTTCTTAGATTCTCCTGAGGCAAAGCCATGGGGC
ATGGCTTTGGGGCTGGGAAGGGGAGTTGTGGCCACAGCCTGTTCCAGGAGAGGGAATCTTGCAGCGGCTG
CATGCAAGTTCAGGGTTCTGCTTAACTTAATTTTACTAATCACACACTTTGTTGGCTTCAGGTCCTTTTT
TGGAGAGATTTTCTTCAGTTAGGTTACACAGATAAAGATTTTAATATCTAGGAATGAAGAAAATTCATTT
CATCAGTTATAAAACTGGCATTATAAAAAGGCTTTTTAGGCGAAATAAATTGTCCAGAGTTGGCAATTCA
AAAGCATGCTGAGAGCCGCTCAGTCCCTGTCCATTGTGGAGGGAAGCGTTTTCCAGAGTGTTCTGAGTCA
TGGGTGTTAAACCTCCATGGTGGGCTGCTTGGTATACTTGGTATAACTAGGAGACATCATTTCACCTGGC
CTCGGTCAAATAATCTCCCTCCTTGCTATTCCCATGAGGTTGTAGGACCTTCCCCATCTCATACATGTTT
GGTAGAGTTGCTATCGAGGGCTTGTTGAGGGGAAGGAGATGGGGCATTGGGGCAGCTGGCCTGAAACTGT
CCCCCGAGAGTTCCCAAGATACATGTGCATATTGGGAAATGGATGGGCTAATGAGGTCCAGTATTTTCAT
GGAACCACTCAGTGTCTCCCGACAGACCTTGGTCCTGTGGGATTTCTCGCCTGCTTCTTTCAACTCTAAA
ATACTAAACTCTGAGCTTCTAGGAGGAGCAGAGGTCCTGCATGCAGGGCCGACTGGGCAGTGCACGCATA
AAGAGTCGGGGACATCTGTCGAGCACCTGATTTGACTGCAGGCCCCGTGGTTCCCACACTGAGATCCTGG
AGCCAGTGCTCAGGGAAGCCTTCCTTAAATATCCTTACATGTCAGAAAAGGTCAGCGTTGAAGGGGACAG
CCAGTCAGTTATGTGGCCTGCACCCTGAGAAGTGACATATTTAATGTCACAGAATTTTTGGTCTTTAGGG
AACTGCAGAATTTAAGTCTGGTTTTTTTTTTTTTTGGAGACAGAGTCTTGCTCTGTCGCCCAGGCTGGAG
TGCAGTGGTGCCATCCTGGCTCACTGCAGCCTCCGCCTCCCAGGTTCAAGCGATTCTCCTGCCTCAGCCT
CCCGAGTAGCTGGGATTACAGGCACCTGCCACCACGTCTGGCTAATTTTTATATTAGTAGAGACCGGGTT
TCACCATGTTGATCAGGATGGACTCAAACTCCTGACCTCAGGTGACCCGCCTGCCTCAGCCTTCCAAAGG
GCTGGGATTGCAGGCATGAGCCACTGCGCCTGGCCCAGAATTTAGTCTTTTAAAGCAGTACATTTAAGGC
TTGCGGTTTGATTAATTTCATCCAGTGGACACACCCGTGTAACAATCACCACAAATCAAGATACAGAACA
TTGTCACCCCCAAAGCTTGCTTCGCAGTGGGACGCTTAAAGCTCAGTGTATTTTAGACTAACTGATGTAA
AGATAGCTGTGCTTTTTTTTTCTTTTTCTGCTAGGTGTTATCTGCCTGTTCACAATTTGAGACCACTTTT
GAATTTACCTTTAAGGATTAACGCTTGGTGGATTTGTGTCTTTGCATGGCTGCAGTGTGTGTTCGTGTGT
TTGCGGGGAGTTTGAAGAGAGTTATGACGTGTGTGCGTGATTTGGGGAAGGGGCTCTTACCCTGTGAACT
AGTGGCTGCTGGGGGAGGGAGGGGAGTCGTGCAGCTGGGGCCCTTCCCGCTGTTATCCAGGTTTCTGTTG
TTACAGTCTCTCTTCTGCCTCCTAGGCCGTTGGCATCCACTGTGAATGATAAGCTGGAGCTGCAGGAGTG
TCTGGAGCATGGCAGGATAGCCAAGGTCAGCTCCAGCGTCTAGAACCTCTGCTGGGGGGCAGGGAGAAGG
AAGGGCAGGATGAGGTGGAGCGGTGGCCAGGGCCTTGTCCCGCCTGCTTCTCCAGCTAGTGTGGAGAATG
CAATTTAGGAGACGGTGCAGGGTCATGCAGCAGGGCAGCCAGAGGAGGGCCCAGGGCTGCTGGCCTTCAG
CCCTTCCTCCCTGGTGAGTGTCGCCCATCACCCCCGGTAGACACTGAGGAGTCTTTACCAAGACCTGGTT
GTGCTGTGGCTTTTTTTTCATTTTTAAATTTTTTGGCCAAAACCTATTACAAAAGCCCTCAAAGCAGCCT
CCTGTGATGTGGAAGCTCCATTTCTCTCTGGAGCTGGAATCTCCCGGAGTGACTACCTGGGGCACTGGGT
TCTGTGTCAGCAGGTTCCAGCATTCAGAACCACTGGCAGCTCCGAGGGCATCTTTGAGCCCCTCAGTAAC
TTGGCATCCTTTAGCCTACAAGTTACTCCATCTCTGAAAGAACTAATTCTTTGGAAGACTTGGAAAGTAC
AGTGGCTACAGCTTCTACTTCTAAAGTCTGTGGCTAGGCCGGGTGCGGTGGCTCACGCCTATAATCCCAA
TACTTTGGGAGGCCGAGGCAGGCGGATCACCTGAGGTCAGGAGTTTGAGACCAGCCTGGCCGACACGGTG
AAACCCTGTCTGACTAAAAATATAAAAATTAGCCAGGCATGGTGGCGGGCGCCTGTAGTCCCAGCTACTC
AGGAGTCTGAGGCATTAGAATCGCTTGAACCCGGGAGGTGGAGGTTACAGTGAGCTGAGATCGCACCACT
GCAGTCCAGCCTGGGTGACAGAGCGAGACTCCGTCTCAAAAAAAAAAAAAAAAGTCTGTGGCTAGATGGA
TCTAAGATTGGCCAGGTGACCTCCAAGTAGGAAGAAAAGTGAGGCTGTGGGGTCTGTGGAGGCTCCTGCC
CTGCGAACTCTGCTCACTCAGAGCCATACCCAGCCCCCTGGGTGGACCAGGCCCTGTCAGGGTTTGGCGA
GTACCTGGCATATTTGGTAGACGCATGACCAGTGTTGGCTGGTGAATGAATTCGTGCTTATTCCTCTTTT
CTCCTCTTCATCTAGTTCAGCAAAGTGAGGACCATTACTACGAGGTCAAACTCCATAAAGCAGGGCAAAG
ACCAGCATTTTCCTGTCTTCATGAATGAGAAAGAGGACATCTTATGGTGCACTGAAATGGAAAGGTACCC
GGGTTGTGCTGGCATCTGGCTGCGGTGTGGCTGTTCTGATTGCTGTGCTTGCTGCGGGGGTCCTCTGCCT
TTCCCACCTGACTTGTTTTCCACAAACGTCCTGGGAACAGTGGGTTTTATCAAGCATTTCCCAACATGGC
AGACTCCTGGGGTGGACTCCTGACACCCCAGGCTCCTGGGTATTTTAAGGCTGGTCCCAAGTAAGAGGAG
TCTAGGGGCCAGGAGCAGTGGCTCATGCCTATAATCCCAGCACTTTGGGAGGCCGAGGCGGGTGGATCAC
CTGAGGTCAGGAGTTCGAGACCAGCCTGGCCAACATGGCGAAACCCCGTCTCTACTAAAAATACAAAAAT
TAGCTGGGTGTGGTGGCGGGTGCCTGTAGTCCCAGCTACTCAGGAGGCTGAGACAGGAGAATCGCTTGAA
CCTGGGAGGTGGAGGTTGCAGTGAGCCGAGATTGCGCCACTGTGCTCCAGCCTGTGCAACAGAGAGATTC
CATCTCAAAGGGAAAAAAAAAAAAAAGAGGAGTCTAGGAAATCCCCTGCTGCCTTGTCTCCCAGAGACCC
TGCTGATCCCCCAGGGAACCACTGCAGTGACAGGACCTTAACTTGTGGTGCCAATTTTATAGGTCTTTTG
TAAACCAAGCCAGCATAGGGCCAGGCAGACCTCGCAGCCTCAACACCCTGATTTGATGCAAATGGGACTT
TCGGGGCTGGATGACCTTCTTGTTCTCTGAGTGTAACTAGGATTTAGCCAGACAAAGAGACCATTTACAC
AAACAACCAGAGCAAACAGGAAATCCTTCAGCTTCTGAAGTCAGGCAGGAGTTCGGCTCCTTTTTCGCCT
TCCCTGCTCTTGATGGGGAGGATGCTAGGGCTACGTGTTCGTTACATGCAGACTCCAGGTGTACGTTGCC
GAGCTAACCCTGCAGGAGAGGAGAACTAGAGAGTGTGTCCTGGAGGTATTTCTGAGGGATTGTTCAAAAT
TACTCTTATTTCTGCTGGGTTGTGAAACTCTAGGCAGTGATGACCTTACTACCTTTAAGGTCACAGAAAC
CAGCACAGTGCCTGGCACATGGTTGGTGATCTGAGTGCCGGGTTGTTTATAAAGGACAGAAGATTCGGCA
GAACTAAGCAGGCGTCAGAGGAGTTGGTGGGTGTGAGTGCCCCTGTCCCTGCACTTCGGGTGGCTGCTGG
TCCTCCGGGTCCTGCTGTGTGGTTAGACGGCTTCCGGGCAGCCTGGTCTGGCCAGCACTCACCCTGCCCT
CTCTGCCTTTTCTCCCCCAGGGTATTTGGTTTCCCAGTCCACTATACTGACGTCTCCAACATGAGCCGCT
TGGCGAGGCAGAGACTGCTGGGCCGGTCATGGAGCGTGCCAGTCATCCGCCACCTCTTCGCTCCGCTGAA
GGAGTATTTTGCGTGTGTGTAAGGGACATGGGGGCAAACTGAGGTAGCGACACAAAGTTAAACAAACAAA
CAAAAAACACAAAACATAATAAAACACCAAGAACATGAGGATGGAGAGAAGTATCAGCACCCAGAAGAGA
AAAAGGAATTTAAAACAAAAACCACAGAGGCGGAAATACCGGAGGGCTTTGCCTTGCGAAAAGGGTTGGA
CATCATCTCCTGATTTTTCAATGTTATTCTTCAGTCCTATTTAAAAACAAAACCAAGCTCCCTTCCCTTC
CTCCCCCTTCCCTTTTTTTTCGGTCAGACCTTTTATTTTCTACTCTTTTCAGAGGGGTTTTCTGTTTGTT
TGGGTTTTGTTTCTTGCTGTGACTGAAACAAGAAGGTTATTGCAGCAAAAATCAGTAACAAAAAATAGTA
ACAATACCTTGCAGAGGAAAGGTGGGAGAGAGGAAAAAAGGAAATTCTATAGAAATCTATATATTGGGTT
GTTTTTTTTTTTGTTTTTTGTTTTTTTTTTTTGGGTTTTTTTTTTTACTATATATCTTTTTTTTGTTGTC
TCTAGCCTGATCAGATAGGAGCACAAGCAGGGGACGGAAAGAGAGAGACACTCAGGCGGCAGCATTCCCT
CCCAGCCACTGAGCTGTCGTGCCAGCACCATTCCTGGTCACGCAAAACAGAACCCAGTTAGCAGCAGGGA
GACGAGAACACCACACAAGACATTTTTCTACAGTATTTCAGGTGCCTACCACACAGGAAACCTTGAAGAA
AATCAGTTTCTAGAAGCCGCTGTTACCTCTTGTTTACAGTTTATATATATATGATAGATATGAGATATAT
ATATAAAAGGTACTGTTAACTACTGTACAACCCGACTTCATAATGGTGCTTTCAAACAGCGAGATGAGTA
AAAACATCAGCTTCCACGTTGCCTTCTGCGCAAAGGGTTTCACCAAGGATGGAGAAAGGGAGACAGCTTG
CAGATGGCGCGTTCTCACGGTGGGCTCTTCCCCTTGGTTTGTAACGAAGTGAAGGAGGAGAACTTGGGAG
CCAGGTTCTCCCTGCCAAAAAGGGGGCTAGATGAGGTGGTCGGGCCCGTGGACAGCTGAGAGTGGGATTC
ATCCAGACTCATGCAATAACCCTTTGATTGTTTTCTAAAAGGAGACTCCCTCGGCAAGATGGCAGAGGGT
ACGGAGTCTTCAGGCCCAGTTTCTCACTTTAGCCAATTCGAGGGCTCCTTGTGGTGGGATCAGAACTAAT
CCAGAGTGTGGGAAAGTGACAGTCAAAACCCCACCTGGAGCAAATAAAAAAACATACAAAACGTACTGGT
GCTTTCCTGTCTAAGTCGCCTTTTGTGTGTTCTTTTATGAGGCCCCCACCATCCACCCCTCTGTGCACAA
GGCGGCTCCCGGCCCCTGGAATGTGATGTTTTTGGTCATCTCCAAAGGCTGCAGTTTTATACTGGGAGGC
TGATGACACCTTTTATTATAATTATTCTTATGGTTCTGGTTATAATTATGTTTGTTTTAAGATTTTCTTT
AAGAAAACAAAAACCCAACACCCTTCCCTTTAGGTTTCAAACCAAGGTGCGGGAGCAGGGTGCTTCTGAG
TTAGCGGTGGCTCTGGTGCCCTGGCTCCCACCCCTCGGGCCAGGTGGGCCACTGGGCATGGTGACAGCTG
GGCGGCCAGGGAGTCCCCAAGGTCTGCCTTCTGGGCTTGTCACTCTTCTCTTCTACTTCCCCTCCTCTTG
AGTCGGGTGTGTCAGGGCTGGAGGGAGGACCGGCCGTCTCTCTCCTTGTGTGTGTGATTGGAGTGGTGTG
TATTTCTTTTCTAGTACTTGGTCTGATGATAGCTGTGCTCTTACCTCGAGTCAGCAGCACCTGGAGCCCC
AAGTGCTCAACACTTTGTTCTACTTCCTACAAGGCAGGCGGCCTGGCGTCAGTAGTAGGAAGGGATGGCA
TTGTTGGCTCCAGGGTGAGGGCCCGACGGGCAGGTGCTTGCCTGGGTAGATGGGGTGTAGATATGTGGCA
TATAGAGATATATATTTTATAATGGGAGGGGGGATGGCACCTCCTGGGACTGGTAGAGCTGGGAGGTGTG
TCGGCAAGCACATGGCCCCAAGTCCACGTCCCTAGAAAGATGGGGCCTACAGTGATAGGCGCTCTATAAA
TACATAGATAGGGTCATGTTTAAGAAATATTATGGGGTGGGGAGTGGGGGAAGCAGGCTTAGTGTGCTGA
GCCTCGGTCCCCACTGCAGACCTAAACCCCACACCAATCAGTCAATCTCACACGCATACACAGGAGCATA
TTTCTGTACTGATGCATAATGAATGCACTCACACACACACACACACGCCAGGGGGCTCCCAGCACTCTTC
TCCCAGGGTTCAGGACTTTGTGGAGTCAGCCCGTGCAGCTATTTACCTCTGTTGCCCTGATTGCAGGTAG
ACTGTCAATCCCAGAAGTTGCTAGGTGCTGAGGCAGGAAAAGGAGGAGGTTTCTTTTAGCAGGTGCTCTC
CTCTAGCAGGTAGTGTTCTATCTCTTTTGCCCCTGGTGATGTCAAAAGATTCTTCTGTTAGCAACCTGTG
ATACTGACCTGCAACTCTAGGCACCAGTCAAGTCAGCCCTCAACTCCCCCTACCTATTCTCCCCAACTGA
AATCAAATTGACATCCAGGTCTGTCTTGGCCAGGGGGCTTCCATTGGGGAAAAAAAATGGGACAGTGAAC
CTCACCTATTGAATAAACCAAATTAGTAATTAGTGACAAAACCAAACCAAGGCTTTGGACCCCCTGCTCA
TTACTCAGGAGTAAATTCACACAGCTGGTGGCCGGTCTCACCCCTTTTTGGGGTGCTTGGTCCTGGACCC
CAATCTACTGTCTCCTCGACAGATTGGTGTTTAAGATCATGCCCAGTTGCTTCCTGTCACCTCTTGACAG
GTGGTGACTGGGCTGCTGTGGGGCTGGAGCAGCGAGGAAGGGAGCCTCTGCCCCCTCAGTGGAGTCCCTG
TGCTAACTGTGGTGCTCACCCAGCTCCTCCTTGCTCCCGCGGGGCTCTGTCCTTCGGGCCTGGAGTCCAC
CAGCGCCACTCCAGGAGGGCTGGGGGAGGAGGGGCTGCCTCAGGCAGTCCAAGGTGCTCCCTCCCCCGCT
GGCCAATGCTAAGGTCTTCCCGGCTCCCAGGGTCAGGACAGAGTCTTCTAAGGCTCAGGCTCTCCCAAGC
CACTGCTTAGTGACAGTTAGCCTTTCCCAGCGGCCCAAGCTCCACCGGAACTGGGCCTGGTGCTCCAACC
AAAGGGTTGGTGTTGGGACTTGGGAAGAGACTTGGTAGCCAAGGGCATGGCAAGCCTGGGGGATTCCTGT
GGGCCGGCTTGCTCGAGGAGGGAGTGCTGGGCACAGAGGAGCCCTTATGGCTGACAGCTTGAGAGCACTT
GTTCCTCCAGATCTAGCCACTTGACCAGGCCCTAGAATGGACCAGATGCCCCAGGGGTCATGGCCCCCCC
CCCCCCCCCCTTGCAGGGACGAGAGTTAACAATCACCAGCTTTTCTTCCGGCCCAGCCTGTGTGCTTGGT
CTCACAGTAGAATTTAGGAGAGTGTGGGGCTTTGGGATTGTGCCAACTTGGCCAAGCTGAGATATGCTTA
ATCCACCTCAACGCTGGGGCCTCTGGGGACAGGTATTGGGTCACCTTCCAAGGGGTGCTGAACTTCAGCA
GGCTGTAGATAAGGCAGAGGTTAGAGGCAGTGGAGTGGGGGAACCAAGAAGGCCTAGGGAAAATTGCCAG
GCATATTCCCTGTGGAATTTTCTTTCCGAAGAGTAGGCCCATTTCCCCCTTAGAAACAAGGGTTCTGGGG
GCTGATGACAGCTTCCCCTGCAGGTGCCAGGTGGCGCATGATGCCAAAGAGATCCGTGGCTCTTGCCTGG
GGGAGGTAGCCAGGTGCTCGTGCTGGGGACACAGTCCTTGGCCTCCTGCCTAAGCAGGAAGCTAGTTTCC
AGCAGGCGGCTGGTACTAACCCCCCGCCCCACGGGAGCGACAGGCTGGTGCACCGGCAGACTGGGGGGCC
GCAGGCGGGCACTAGCAGGGGTCTGCAGTCCATAGATGATGACATGAATCTGTTGCTGTCCTTTTGTCTC
TAGGCCCCAGAGACTTCTTGCTATTGAACATCCTATTGGAAAGCAAACTTTCCAAGAGAGCACCCGGGAC
CTGTTAGCTTTTGAAACAGGCCTGGGTGTGATTGACTGGTAGAGATGCCTGTGTGATTTTTGGCGCATTG
CAGGAGTGTGGATTGGGACAGGCACCCCAGCAGGAAGGAAGGCTGACACCAAAGGGGTCTCACAGGGCCC
AGCTCCCCTTGCTTCCCGATAATTGGCCACCAGGCCATGGCGACTCTGGGGAAGCCTAGATGGGAACACC
CACCTTCTCCAGCAGTAGAAGGAATATTTCAGCAGAGCCATCTGGGAGATGTTCTTATAGGAAAACTGCA
GTCATTCATTTCATAGTTTTGCTTATATAAATAGAGTACATTTGGGAGGCAGTTTTGAATTCCTGTTGCT
ACTCTGCTTGGTGCGGAGTTGCAGTGGCCCCAAGTGTCTTTCAGAGCTGTTGCTTTTAGGATCCCCCTCA
AGCTGTGGGTTAGGGCCAGGGCAGGGAGGGGGTGCTGCCTGGCACTGTGCCCTGAAGAGGGCTGCTTGCT
TCCTTTGGTCGGGAGCACGGCAGAGCCAACATTCTGGTGGGAACCGAGTTTCTAGCCAGGCCCCTGTTTT
GGTCCCGGGAGGTGAGTAATCTGAGTTTGTCATTGCGTATTCTCCACGCTGCCTGCTCTCAGTGCTGGAG
ACTAGAGAGCGACCCTGGAGTTGTCCTCTGGGAGTCCTGCCTGTTTGCCAGGGAGCAGCTGAAACCTCCC
CGCTCCCAATCCCGGGGGTGGAAAAGGTGTAGTGGAGGAATGGTCTGGAGCTGGGATGGCCCTTCTGGCC
ATGCCCTGCTGAGCCAGGATTAAGGAAAGTGTCAGCCGCCTCCTTACCAGGCCCTGTGCTGGGGTCAGGA
GGACCGGGAGGAGAGGGAAGGGGTGAGAGGATGGACTCTGCTGTTGTAGTGTGAGCTCTGGAATCTTTAC
GTTTTGGTAAAGGCAGGCCAGGGTCTTTGAGGATCGAGTTGGGGAACCTTGCCTCTGTGTTTCCGAGACC
CAGCTAAGGTGGGCCTTTGGTGGGTGACCTGTCTCTGACCCGAGGCAGCAGAGTTTTGGGGGAGGGAGAA
ACAAGCAAGGAGCAGGGAGGACAAGGACACAAGATCCCTTTGCTGGTGGTGACTTACTCACTTCCCCCAC
CAGAGGGCCTGGGACTCCCGAAACTCCCGTAACTGGAAAGCTCCCTGGCTCAGCTCCACTGGGGGGTGAG
GAGGGCGGCTCCCTCAGCCCCAGCCTAGCGGCCTCCAACCCTGTGCCTGAGAACATGAGCAGCGGTCTAA
AGCATCCCCTAGTCCCATTTCACTTCACAGACCGATTTGGTTGCACAGCCCTAGAATTCAACAGACAACA
AAAATGCTGAAATTCCTTAGAATACGCAGAGGGAGGAGGTGACTCACCAAGGTGCTAAAACATTTTATTA
AAAATATATATTGTTAAATTAAGTCTGCTGTCTGGACAATGACTGTTTGTTTTGTTTTCTTGTAGTGGAG
TTTAAAAGAGACTATTATTTTACTCTGATATATTATTATTAAAAAGGCATTTTAACTTTGTACTTGAAAA
CTAAATGAGCGATTTCATGTGTTTTAGCTGAGGCATTGAAGTAGCGGGTGCTTACTTATTTGTGGGAAAT
CATGTATTCATACTGAAGAATAAACTCCGTAGCTGATTTGGTAATAACTTTTACTGTTACAGACGATTTC
GCTTTGACCCCGGTGGCAGAGCACTTTTACTCCACATTCCACGCAGCTAAATGCAGGACTTCCCAGACTC
TTCCCTCCCCAGCCCTCTTTCCTTTCTCTTAAGTTCCTCTTTTCTCCTTTATTATTATTATTTTTTTTTC
CTGGTTCCAGCATCCTTTCACCACTGTATTTTTTTTTTTTTTTTTTTTTTTTTTTTTTTTTTTTTTTTTT
TTTTTTTTTTAGGGTTGCTTCTCTATTTATTGACAATAATAATGTACATTTCACAGTCTGGTTCTAGGAC
ATTCAGGGAAGGGCGAGTGGGGGCCCGAGGGGTCCCTGCCCCACCCGCCTGGTGTCCTGTTGTATCTCTG
TGTAAGTGATGCTCGTGACATAGCTGTTTATGAAATAATTAAAGAGGTCAATGCGTACAACAGATGTAGA
AAGTCTGTCAGTTCCGCCTTCATCACATTTTTTTTTGTGCCCAGAAGAATATAATAAAGCTCCTTTCTAA
TGTACTTGTGCTGGAGAACACTTGAATAAATGGACTGTTTTTGTGCAAAAAGAAAAA
SEQ ID NO: 28 (NM_001320892.2, DNMT3A isoform c mRNA):
GCGCGGGCGGCGGCGGCGGCGAGAGCAGAGGACGAGCCGGGACGCGGCGCCGCGGCACCAGGGCGCGCAG
CCGGGCCGGCCCGACCCCACCGGCCATACGGTGGAGCCATCGAAGCCCCCACCCACAGGCTGACAGAGGC
ACCGTTCACCAGAGGGCTCAACACCGGGATCTATGTTTAAGTTTTAACTCTCGCCTCCAAAGACCACGAT
AATTCCTTCCCCAAAGCCCAGCAGCCCCCCAGCCCCGCGCAGCCCCAGCCTGCCTCCCGGCGCCCAGATG
CCCGCCATGCCCTCCAGCGGCCCCGGGGACACCAGCAGCTCTGCTGCGGAGCGGGAGGAGGACCGAAAGG
ACGGAGAGGAGCAGGAGGAGCCGCGTGGCAAGGAGGAGCGCCAAGAGCCCAGCACCACGGCACGGAAGGT
GGGGCGGCCTGGGAGGAAGCGCAAGCACCCCCCGGTGGAAAGCGGTGACACGCCAAAGGACCCTGCGGTG
ATCTCCAAGTCCCCATCCATGGCCCAGGACTCAGGCGCCTCAGAGCTATTACCCAATGGGGACTTGGAGA
AGCGGAGTGAGCCCCAGCCAGAGGAGGGGAGCCCTGCTGGGGGGCAGAAGGGCGGGGCCCCAGCAGAGGG
AGAGGGTGCAGCTGAGACCCTGCCTGAAGCCTCAAGAGCAGTGGAAAATGGCTGCTGCACCCCCAAGGAG
GGCCGAGGAGCCCCTGCAGAAGCGGGTGAGTCCTCAGCACCAGGGGCAGCCTCTTCTGGGCCCACCAGCA
TACCCTGAGAGTCAGGGACTTGGCTCTCCAGCAGGTCCCAGGAAGGATGGTCTGGGTCGTGGCTAAAGGT
CTGCTTGCCAAGGCTATGGCCTGGAGGCTACTGGCTGGATGCAGCCTGCGCATATGTTTTATTTGGCCCA
TAGAGTGTTTTAAACATTTAAAAAATTAGTTGCCAGTATTTAAAAATCAAAAAATTTCACATAAAAATCT
GGAGTTTTGGCTTCTCATGAAAAAAAAAAAAGCTAGATCTGGCAACAGCGGGCTTTCATAACGCCAACGA
TTGCTAGACTGGGATAATGGCGGTCCCTCCATCGCCTTCTGTGGCTGGTTGTGGGCCTTAGTTTTCTGCA
GCTCTACCTGGCCTGCTTACTCTCCCACGTGCCATGCAGTTCCTGGGGGTTGCTGTATTTGTAGCCCCTG
GCCTGGGCACTCAAGGGCAGCAGATACCCTGTTTGCCTCCCTGAGTGCAGAGGTCCTGAGCCCACCCTAG
TTGGGCTGACTCAACTGGAAATTTGGTTGTGACAGTGGCGTGGGGAGAGGGCTGGGTGATTGTATTCTGT
GTACTGCCCAGCCCAGGCCTCTTCATCTGGGGACTTTTTGGCCTAACCCTGGAAGCCTGGAAAGTTGCCC
ACTTTTCTCTTTCAGGTTAAGCCAGCAATTTCAGGGCCAACCGAGCTGTAAACATGTTAGTAATGAGGAC
AACTAGCATTTGTACAGGGCTTCACAGTTTACAAAGCGCTTTCTCATACATTATCACATTTGATCCTCCC
AGGGCCCTGCCAGGTTGTTTTGCATATGTGCATTTTAATTTCAAAAAGTCTTCCTTCCAAGCGTGTATGA
TGAAATGAGTAAATTGATTAATTGGCGTAACTTATTTTGCATGGATCCAACCTAATGTTCATGCAGGATA
GAGAACATTTCCAGAATACAAATTTCCAAACTTA
SEQ ID NO: 29 (NM_001320893.1, DNMT3A isoform d mRNA):
GATCTCCGCAGCCCTCCGGGTTTGAAAAGAGTGAAGTATGCCTCCCCGGCCCGCAGCAGCCGCGGCGTCC
CGGCCTGGCCCGAGTTCTGGAGATGCTGACTTAGTGCTGGCCCGCCGCCTTCCTGCCCACGAGCCCCAAG
GCCCCACTCCTCGAGGGCTCCAGTGGCACCTGGCGCCCTCGGGGGACAGGAAGAGCAGCCGGCCGGGAGC
CCGGGCAGTGGCTGAGAAGAAAGCCAAGGTCATTGCAGGAATGAATGCTGTGGAAGAAAACCAGGGGCCC
GGGGAGTCTCAGAAGGTGGAGGAGGCCAGCCCTCCTGCTGTGCAGCAGCCCACTGACCCCGCATCCCCCA
CTGTGGCTACCACGCCTGAGCCCGTGGGGTCCGATGCTGGGGACAAGAATGCCACCAAAGCAGGCGATGA
CGAGCCAGAGTACGAGGACGGCCGGGGCTTTGGCATTGGGGAGCTGGTGTGGGGGAAACTGCGGGGCTTC
TCCTGGTGGCCAGGCCGCATTGTGTCTTGGTGGATGACGGGCCGGAGCCGAGCAGCTGAAGGCACCCGCT
GGGTCATGTGGTTCGGAGACGGCAAATTCTCAGTGGTGTGTGTTGAGAAGCTGATGCCGCTGAGCTCGTT
TTGCAGTGCGTTCCACCAGGCCACGTACAACAAGCAGCCCATGTACCGCAAAGCCATCTACGAGGTCCTG
CAGGTGGCCAGCAGCCGCGCGGGGAAGCTGTTCCCGGTGTGCCACGACAGCGATGAGAGTGACACTGCCA
AGGCCGTGGAGGTGCAGAACAAGCCCATGATTGAATGGGCCCTGGGGGGCTTCCAGCCTTCTGGCCCTAA
GGGCCTGGAGCCACCAGAAGAAGAGAAGAATCCCTACAAAGAAGTGTACACGGACATGTGGGTGGAACCT
GAGGCAGCTGCCTACGCACCACCTCCACCAGCCAAAAAGCCCCGGAAGAGCACAGCGGAGAAGCCCAAGG
TCAAGGAGATTATTGATGAGCGCACAAGAGAGCGGCTGGTGTACGAGGTGCGGCAGAAGTGCCGGAACAT
TGAGGACATCTGCATCTCCTGTGGGAGCCTCAATGTTACCCTGGAACACCCCCTCTTCGTTGGAGGAATG
TGCCAAAACTGCAAGAACTGCTTTCTGGAGTGTGCGTACCAGTACGACGACGACGGCTACCAGTCCTACT
GCACCATCTGCTGTGGGGGCCGTGAGGTGCTCATGTGCGGAAACAACAACTGCTGCAGGTGCTTTTGCGT
GGAGTGTGTGGACCTCTTGGTGGGGCCGGGGGCTGCCCAGGCAGCCATTAAGGAAGACCCCTGGAACTGC
TACATGTGCGGGCACAAGGGTACCTACGGGCTGCTGCGGCGGCGAGAGGACTGGCCCTCCCGGCTCCAGA
TGTTCTTCGCTAATAACCACGACCAGGAATTTGACCCTCCAAAGGTTTACCCACCTGTCCCAGCTGAGAA
GAGGAAGCCCATCCGGGTGCTGTCTCTCTTTGATGGAATCGCTACAGGGCTCCTGGTGCTGAAGGACTTG
GGCATTCAGGTGGACCGCTACATTGCCTCGGAGGTGTGTGAGGACTCCATCACGGTGGGCATGGTGCGGC
ACCAGGGGAAGATCATGTACGTCGGGGACGTCCGCAGCGTCACACAGAAGCATATCCAGGAGTGGGGCCC
ATTCGATCTGGTGATTGGGGGCAGTCCCTGCAATGACCTCTCCATCGTCAACCCTGCTCGCAAGGGCCTC
TACGAGGGCACTGGCCGGCTCTTCTTTGAGTTCTACCGCCTCCTGCATGATGCGCGGCCCAAGGAGGGAG
ATGATCGCCCCTTCTTCTGGCTCTTTGAGAATGTGGTGGCCATGGGCGTTAGTGACAAGAGGGACATCTC
GCGATTTCTCGAGTCCAACCCTGTGATGATTGATGCCAAAGAAGTGTCAGCTGCACACAGGGCCCGCTAC
TTCTGGGGTAACCTTCCCGGTATGAACAGGCCGTTGGCATCCACTGTGAATGATAAGCTGGAGCTGCAGG
AGTGTCTGGAGCATGGCAGGATAGCCAAGTTCAGCAAAGTGAGGACCATTACTACGAGGTCAAACTCCAT
AAAGCAGGGCAAAGACCAGCATTTTCCTGTCTTCATGAATGAGAAAGAGGACATCTTATGGTGCACTGAA
ATGGAAAGGGTATTTGGTTTCCCAGTCCACTATACTGACGTCTCCAACATGAGCCGCTTGGCGAGGCAGA
GACTGCTGGGCCGGTCATGGAGCGTGCCAGTCATCCGCCACCTCTTCGCTCCGCTGAAGGAGTATTTTGC
GTGTGTGTAAGGGACATGGGGGCAAACTGAGGTAGCGACACAAAGTTAAACAAACAAACAAAAAACACAA
AACATAATAAAACACCAAGAACATGAGGATGGAGAGAAGTATCAGCACCCAGAAGAGAAAAAGGAATTTA
AAACAAAAACCACAGAGGCGGAAATACCGGAGGGCTTTGCCTTGCGAAAAGGGTTGGACATCATCTCCTG
ATTTTTCAATGTTATTCTTCAGTCCTATTTAAAAACAAAACCAAGCTCCCTTCCCTTCCTCCCCCTTCCC
TTTTTTTTCGGTCAGACCTTTTATTTTCTACTCTTTTCAGAGGGGTTTTCTGTTTGTTTGGGTTTTGTTT
CTTGCTGTGACTGAAACAAGAAGGTTATTGCAGCAAAAATCAGTAACAAAAAATAGTAACAATACCTTGC
AGAGGAAAGGTGGGAGAGAGGAAAAAAGGAAATTCTATAGAAATCTATATATTGGGTTGTTTTTTTTTTT
GTTTTTTGTTTTTTTTTTTTGGGTTTTTTTTTTTACTATATATCTTTTTTTTGTTGTCTCTAGCCTGATC
AGATAGGAGCACAAGCAGGGGACGGAAAGAGAGAGACACTCAGGCGGCAGCATTCCCTCCCAGCCACTGA
GCTGTCGTGCCAGCACCATTCCTGGTCACGCAAAACAGAACCCAGTTAGCAGCAGGGAGACGAGAACACC
ACACAAGACATTTTTCTACAGTATTTCAGGTGCCTACCACACAGGAAACCTTGAAGAAAATCAGTTTCTA
GAAGCCGCTGTTACCTCTTGTTTACAGTTTATATATATATGATAGATATGAGATATATATATAAAAGGTA
CTGTTAACTACTGTACAACCCGACTTCATAATGGTGCTTTCAAACAGCGAGATGAGTAAAAACATCAGCT
TCCACGTTGCCTTCTGCGCAAAGGGTTTCACCAAGGATGGAGAAAGGGAGACAGCTTGCAGATGGCGCGT
TCTCACGGTGGGCTCTTCCCCTTGGTTTGTAACGAAGTGAAGGAGGAGAACTTGGGAGCCAGGTTCTCCC
TGCCAAAAAGGGGGCTAGATGAGGTGGTCGGGCCCGTGGACAGCTGAGAGTGGGATTCATCCAGACTCAT
GCAATAACCCTTTGATTGTTTTCTAAAAGGAGACTCCCTCGGCAAGATGGCAGAGGGTACGGAGTCTTCA
GGCCCAGTTTCTCACTTTAGCCAATTCGAGGGCTCCTTGTGGTGGGATCAGAACTAATCCAGAGTGTGGG
AAAGTGACAGTCAAAACCCCACCTGGAGCAAATAAAAAAACATACAAAACGTACTGGTGCTTTCCTGT
SEQ ID NO: 30 (NM_001375819.1, DNMT3A isoform e mRNA):
ACTTCCAGAGCTTCAGGGCCGCGGCTCACACCTGAGCGCGACTGCAGAGGGGCTGCACCTGGCCTTATGG
GCTGAGAAGAAAGCCAAGGTCATTGCAGGAATGAATGCTGTGGAAGAAAACCAGGGGCCCGGGGAGTCTC
AGAAGGTGGAGGAGGCCAGCCCTCCTGCTGTGCAGCAGCCCACTGACCCCGCATCCCCCACTGTGGCTAC
CACGCCTGAGCCCGTGGGGTCCGATGCTGGGGACAAGAATGCCACCAAAGCAGGCGATGACGAGCCAGAG
TACGAGGACGGCCGGGGCTTTGGCATTGGGGAGCTGGTGTGGGGGAAACTGCGGGGCTTCTCCTGGTGGC
CAGGCCGCATTGTGTCTTGGTGGATGACGGGCCGGAGCCGAGCAGCTGAAGGCACCCGCTGGGTCATGTG
GTTCGGAGACGGCAAATTCTCAGTGGTGTGTGTTGAGAAGCTGATGCCGCTGAGCTCGTTTTGCAGTGCG
TTCCACCAGGCCACGTACAACAAGCAGCCCATGTACCGCAAAGCCATCTACGAGGTCCTGCAGGTGGCCA
GCAGCCGCGCGGGGAAGCTGTTCCCGGTGTGCCACGACAGCGATGAGAGTGACACTGCCAAGGCCGTGGA
GGTGCAGAACAAGCCCATGATTGAATGGGCCCTGGGGGGCTTCCAGCCTTCTGGCCCTAAGGGCCTGGAG
CCACCAGAAGAAGAGAAGAATCCCTACAAAGAAGTGTACACGGACATGTGGGTGGAACCTGAGGCAGCTG
CCTACGCACCACCTCCACCAGCCAAAAAGCCCCGGAAGAGCACAGCGGAGAAGCCCAAGGTCAAGGAGAT
TATTGATGAGCGCACAAGAGAGCGGCTGGTGTACGAGGTGCGGCAGAAGTGCCGGAACATTGAGGACATC
TGCATCTCCTGTGGGAGCCTCAATGTTACCCTGGAACACCCCCTCTTCGTTGGAGGAATGTGCCAAAACT
GCAAGAACTGCTTTCTGGAGTGTGCGTACCAGTACGACGACGACGGCTACCAGTCCTACTGCACCATCTG
CTGTGGGGGCCGTGAGGTGCTCATGTGCGGAAACAACAACTGCTGCAGGTGCTTTTGCGTGGAGTGTGTG
GACCTCTTGGTGGGGCCGGGGGCTGCCCAGGCAGCCATTAAGGAAGACCCCTGGAACTGCTACATGTGCG
GGCACAAGGGTACCTACGGGCTGCTGCGGCGGCGAGAGGACTGGCCCTCCCGGCTCCAGATGTTCTTCGC
TAATAACCACGACCAGGAATTTGACCCTCCAAAGGTTTACCCACCTGTCCCAGCTGAGAAGAGGAAGCCC
ATCCGGGTGCTGTCTCTCTTTGATGGAATCGCTACAGGGCTCCTGGTGCTGAAGGACTTGGGCATTCAGG
TGGACCGCTACATTGCCTCGGAGGTGTGTGAGGACTCCATCACGGTGGGCATGGTGCGGCACCAGGGGAA
GATCATGTACGTCGGGGACGTCCGCAGCGTCACACAGAAGCATATCCAGGAGTGGGGCCCATTCGATCTG
GTGATTGGGGGCAGTCCCTGCAATGACCTCTCCATCGTCAACCCTGCTCGCAAGGGCCTCTACGAGGGCA
CTGGCCGGCTCTTCTTTGAGTTCTACCGCCTCCTGCATGATGCGCGGCCCAAGGAGGGAGATGATCGCCC
CTTCTTCTGGCTCTTTGAGAATGTGGTGGCCATGGGCGTTAGTGACAAGAGGGACATCTCGCGATTTCTC
GAGTCCAACCCTGTGATGATTGATGCCAAAGAAGTGTCAGCTGCACACAGGGCCCGCTACTTCTGGGGTA
ACCTTCCCGGTATGAACAGGCCGTTGGCATCCACTGTGAATGATAAGCTGGAGCTGCAGGAGTGTCTGGA
GCATGGCAGGATAGCCAAGTTCAGCAAAGTGAGGACCATTACTACGAGGTCAAACTCCATAAAGCAGGGC
AAAGACCAGCATTTTCCTGTCTTCATGAATGAGAAAGAGGACATCTTATGGTGCACTGAAATGGAAAGGG
TATTTGGTTTCCCAGTCCACTATACTGACGTCTCCAACATGAGCCGCTTGGCGAGGCAGAGACTGCTGGG
CCGGTCATGGAGCGTGCCAGTCATCCGCCACCTCTTCGCTCCGCTGAAGGAGTATTTTGCGTGTGTGTAA
GGGACATGGGGGCAAACTGAGGTAGCGACACAAAGTTAAACAAACAAACAAAAAACACAAAACATAATAA
AACACCAAGAACATGAGGATGGAGAGAAGTATCAGCACCCAGAAGAGAAAAAGGAATTTAAAACAAAAAC
CACAGAGGCGGAAATACCGGAGGGCTTTGCCTTGCGAAAAGGGTTGGACATCATCTCCTGATTTTTCAAT
GTTATTCTTCAGTCCTATTTAAAAACAAAACCAAGCTCCCTTCCCTTCCTCCCCCTTCCCTTTTTTTTCG
GTCAGACCTTTTATTTTCTACTCTTTTCAGAGGGGTTTTCTGTTTGTTTGGGTTTTGTTTCTTGCTGTGA
CTGAAACAAGAAGGTTATTGCAGCAAAAATCAGTAACAAAAAATAGTAACAATACCTTGCAGAGGAAAGG
TGGGAGAGAGGAAAAAAGGAAATTCTATAGAAATCTATATATTGGGTTGTTTTTTTTTTTGTTTTTTGTT
TTTTTTTTTTGGGTTTTTTTTTTTACTATATATCTTTTTTTTGTTGTCTCTAGCCTGATCAGATAGGAGC
ACAAGCAGGGGACGGAAAGAGAGAGACACTCAGGCGGCAGCATTCCCTCCCAGCCACTGAGCTGTCGTGC
CAGCACCATTCCTGGTCACGCAAAACAGAACCCAGTTAGCAGCAGGGAGACGAGAACACCACACAAGACA
TTTTTCTACAGTATTTCAGGTGCCTACCACACAGGAAACCTTGAAGAAAATCAGTTTCTAGAAGCCGCTG
TTACCTCTTGTTTACAGTTTATATATATATGATAGATATGAGATATATATATAAAAGGTACTGTTAACTA
CTGTACAACCCGACTTCATAATGGTGCTTTCAAACAGCGAGATGAGTAAAAACATCAGCTTCCACGTTGC
CTTCTGCGCAAAGGGTTTCACCAAGGATGGAGAAAGGGAGACAGCTTGCAGATGGCGCGTTCTCACGGTG
GGCTCTTCCCCTTGGTTTGTAACGAAGTGAAGGAGGAGAACTTGGGAGCCAGGTTCTCCCTGCCAAAAAG
GGGGCTAGATGAGGTGGTCGGGCCCGTGGACAGCTGAGAGTGGGATTCATCCAGACTCATGCAATAACCC
TTTGATTGTTTTCTAAAAGGAGACTCCCTCGGCAAGATGGCAGAGGGTACGGAGTCTTCAGGCCCAGTTT
CTCACTTTAGCCAATTCGAGGGCTCCTTGTGGTGGGATCAGAACTAATCCAGAGTGTGGGAAAGTGACAG
TCAAAACCCCACCTGGAGCAAATAAAAAAACATACAAAACGTA
SEQ ID NO: 31 (NM_022552.5, DNMT3A isoform a mRNA):
GCGCGGGCGGCGGCGGCGGCGAGAGCAGAGGACGAGCCGGGACGCGGCGCCGCGGCACCAGGGCGCGCAG
CCGGGCCGGCCCGACCCCACCGGCCATACGGTGGAGCCATCGAAGCCCCCACCCACAGGCTGACAGAGGC
ACCGTTCACCAGAGGGCTCAACACCGGGATCTATGTTTAAGTTTTAACTCTCGCCTCCAAAGACCACGAT
AATTCCTTCCCCAAAGCCCAGCAGCCCCCCAGCCCCGCGCAGCCCCAGCCTGCCTCCCGGCGCCCAGATG
CCCGCCATGCCCTCCAGCGGCCCCGGGGACACCAGCAGCTCTGCTGCGGAGCGGGAGGAGGACCGAAAGG
ACGGAGAGGAGCAGGAGGAGCCGCGTGGCAAGGAGGAGCGCCAAGAGCCCAGCACCACGGCACGGAAGGT
GGGGCGGCCTGGGAGGAAGCGCAAGCACCCCCCGGTGGAAAGCGGTGACACGCCAAAGGACCCTGCGGTG
ATCTCCAAGTCCCCATCCATGGCCCAGGACTCAGGCGCCTCAGAGCTATTACCCAATGGGGACTTGGAGA
AGCGGAGTGAGCCCCAGCCAGAGGAGGGGAGCCCTGCTGGGGGGCAGAAGGGCGGGGCCCCAGCAGAGGG
AGAGGGTGCAGCTGAGACCCTGCCTGAAGCCTCAAGAGCAGTGGAAAATGGCTGCTGCACCCCCAAGGAG
GGCCGAGGAGCCCCTGCAGAAGCGGGCAAAGAACAGAAGGAGACCAACATCGAATCCATGAAAATGGAGG
GCTCCCGGGGCCGGCTGCGGGGTGGCTTGGGCTGGGAGTCCAGCCTCCGTCAGCGGCCCATGCCGAGGCT
CACCTTCCAGGCGGGGGACCCCTACTACATCAGCAAGCGCAAGCGGGACGAGTGGCTGGCACGCTGGAAA
AGGGAGGCTGAGAAGAAAGCCAAGGTCATTGCAGGAATGAATGCTGTGGAAGAAAACCAGGGGCCCGGGG
AGTCTCAGAAGGTGGAGGAGGCCAGCCCTCCTGCTGTGCAGCAGCCCACTGACCCCGCATCCCCCACTGT
GGCTACCACGCCTGAGCCCGTGGGGTCCGATGCTGGGGACAAGAATGCCACCAAAGCAGGCGATGACGAG
CCAGAGTACGAGGACGGCCGGGGCTTTGGCATTGGGGAGCTGGTGTGGGGGAAACTGCGGGGCTTCTCCT
GGTGGCCAGGCCGCATTGTGTCTTGGTGGATGACGGGCCGGAGCCGAGCAGCTGAAGGCACCCGCTGGGT
CATGTGGTTCGGAGACGGCAAATTCTCAGTGGTGTGTGTTGAGAAGCTGATGCCGCTGAGCTCGTTTTGC
AGTGCGTTCCACCAGGCCACGTACAACAAGCAGCCCATGTACCGCAAAGCCATCTACGAGGTCCTGCAGG
TGGCCAGCAGCCGCGCGGGGAAGCTGTTCCCGGTGTGCCACGACAGCGATGAGAGTGACACTGCCAAGGC
CGTGGAGGTGCAGAACAAGCCCATGATTGAATGGGCCCTGGGGGGCTTCCAGCCTTCTGGCCCTAAGGGC
CTGGAGCCACCAGAAGAAGAGAAGAATCCCTACAAAGAAGTGTACACGGACATGTGGGTGGAACCTGAGG
CAGCTGCCTACGCACCACCTCCACCAGCCAAAAAGCCCCGGAAGAGCACAGCGGAGAAGCCCAAGGTCAA
GGAGATTATTGATGAGCGCACAAGAGAGCGGCTGGTGTACGAGGTGCGGCAGAAGTGCCGGAACATTGAG
GACATCTGCATCTCCTGTGGGAGCCTCAATGTTACCCTGGAACACCCCCTCTTCGTTGGAGGAATGTGCC
AAAACTGCAAGAACTGCTTTCTGGAGTGTGCGTACCAGTACGACGACGACGGCTACCAGTCCTACTGCAC
CATCTGCTGTGGGGGCCGTGAGGTGCTCATGTGCGGAAACAACAACTGCTGCAGGTGCTTTTGCGTGGAG
TGTGTGGACCTCTTGGTGGGGCCGGGGGCTGCCCAGGCAGCCATTAAGGAAGACCCCTGGAACTGCTACA
TGTGCGGGCACAAGGGTACCTACGGGCTGCTGCGGCGGCGAGAGGACTGGCCCTCCCGGCTCCAGATGTT
CTTCGCTAATAACCACGACCAGGAATTTGACCCTCCAAAGGTTTACCCACCTGTCCCAGCTGAGAAGAGG
AAGCCCATCCGGGTGCTGTCTCTCTTTGATGGAATCGCTACAGGGCTCCTGGTGCTGAAGGACTTGGGCA
TTCAGGTGGACCGCTACATTGCCTCGGAGGTGTGTGAGGACTCCATCACGGTGGGCATGGTGCGGCACCA
GGGGAAGATCATGTACGTCGGGGACGTCCGCAGCGTCACACAGAAGCATATCCAGGAGTGGGGCCCATTC
GATCTGGTGATTGGGGGCAGTCCCTGCAATGACCTCTCCATCGTCAACCCTGCTCGCAAGGGCCTCTACG
AGGGCACTGGCCGGCTCTTCTTTGAGTTCTACCGCCTCCTGCATGATGCGCGGCCCAAGGAGGGAGATGA
TCGCCCCTTCTTCTGGCTCTTTGAGAATGTGGTGGCCATGGGCGTTAGTGACAAGAGGGACATCTCGCGA
TTTCTCGAGTCCAACCCTGTGATGATTGATGCCAAAGAAGTGTCAGCTGCACACAGGGCCCGCTACTTCT
GGGGTAACCTTCCCGGTATGAACAGGCCGTTGGCATCCACTGTGAATGATAAGCTGGAGCTGCAGGAGTG
TCTGGAGCATGGCAGGATAGCCAAGTTCAGCAAAGTGAGGACCATTACTACGAGGTCAAACTCCATAAAG
CAGGGCAAAGACCAGCATTTTCCTGTCTTCATGAATGAGAAAGAGGACATCTTATGGTGCACTGAAATGG
AAAGGGTATTTGGTTTCCCAGTCCACTATACTGACGTCTCCAACATGAGCCGCTTGGCGAGGCAGAGACT
GCTGGGCCGGTCATGGAGCGTGCCAGTCATCCGCCACCTCTTCGCTCCGCTGAAGGAGTATTTTGCGTGT
GTGTAAGGGACATGGGGGCAAACTGAGGTAGCGACACAAAGTTAAACAAACAAACAAAAAACACAAAACA
TAATAAAACACCAAGAACATGAGGATGGAGAGAAGTATCAGCACCCAGAAGAGAAAAAGGAATTTAAAAC
AAAAACCACAGAGGCGGAAATACCGGAGGGCTTTGCCTTGCGAAAAGGGTTGGACATCATCTCCTGATTT
TTCAATGTTATTCTTCAGTCCTATTTAAAAACAAAACCAAGCTCCCTTCCCTTCCTCCCCCTTCCCTTTT
TTTTCGGTCAGACCTTTTATTTTCTACTCTTTTCAGAGGGGTTTTCTGTTTGTTTGGGTTTTGTTTCTTG
CTGTGACTGAAACAAGAAGGTTATTGCAGCAAAAATCAGTAACAAAAAATAGTAACAATACCTTGCAGAG
GAAAGGTGGGAGAGAGGAAAAAAGGAAATTCTATAGAAATCTATATATTGGGTTGTTTTTTTTTTTGTTT
TTTGTTTTTTTTTTTTGGGTTTTTTTTTTTACTATATATCTTTTTTTTGTTGTCTCTAGCCTGATCAGAT
AGGAGCACAAGCAGGGGACGGAAAGAGAGAGACACTCAGGCGGCAGCATTCCCTCCCAGCCACTGAGCTG
TCGTGCCAGCACCATTCCTGGTCACGCAAAACAGAACCCAGTTAGCAGCAGGGAGACGAGAACACCACAC
AAGACATTTTTCTACAGTATTTCAGGTGCCTACCACACAGGAAACCTTGAAGAAAATCAGTTTCTAGAAG
CCGCTGTTACCTCTTGTTTACAGTTTATATATATATGATAGATATGAGATATATATATAAAAGGTACTGT
TAACTACTGTACAACCCGACTTCATAATGGTGCTTTCAAACAGCGAGATGAGTAAAAACATCAGCTTCCA
CGTTGCCTTCTGCGCAAAGGGTTTCACCAAGGATGGAGAAAGGGAGACAGCTTGCAGATGGCGCGTTCTC
ACGGTGGGCTCTTCCCCTTGGTTTGTAACGAAGTGAAGGAGGAGAACTTGGGAGCCAGGTTCTCCCTGCC
AAAAAGGGGGCTAGATGAGGTGGTCGGGCCCGTGGACAGCTGAGAGTGGGATTCATCCAGACTCATGCAA
TAACCCTTTGATTGTTTTCTAAAAGGAGACTCCCTCGGCAAGATGGCAGAGGGTACGGAGTCTTCAGGCC
CAGTTTCTCACTTTAGCCAATTCGAGGGCTCCTTGTGGTGGGATCAGAACTAATCCAGAGTGTGGGAAAG
TGACAGTCAAAACCCCACCTGGAGCAAATAAAAAAACATACAAAACGTACTGGTGCTTTCCTGTCTAAGT
CGCCTTTTGTGTGTTCTTTTATGAGGCCCCCACCATCCACCCCTCTGTGCACAAGGCGGCTCCCGGCCCC
TGGAATGTGATGTTTTTGGTCATCTCCAAAGGCTGCAGTTTTATACTGGGAGGCTGATGACACCTTTTAT
TATAATTATTCTTATGGTTCTGGTTATAATTATGTTTGTTTTAAGATTTTCTTTAAGAAAACAAAAACCC
AACACCCTTCCCTTTAGGTTTCAAACCAAGGTGCGGGAGCAGGGTGCTTCTGAGTTAGCGGTGGCTCTGG
TGCCCTGGCTCCCACCCCTCGGGCCAGGTGGGCCACTGGGCATGGTGACAGCTGGGCGGCCAGGGAGTCC
CCAAGGTCTGCCTTCTGGGCTTGTCACTCTTCTCTTCTACTTCCCCTCCTCTTGAGTCGGGTGTGTCAGG
GCTGGAGGGAGGACCGGCCGTCTCTCTCCTTGTGTGTGTGATTGGAGTGGTGTGTATTTCTTTTCTAGTA
CTTGGTCTGATGATAGCTGTGCTCTTACCTCGAGTCAGCAGCACCTGGAGCCCCAAGTGCTCAACACTTT
GTTCTACTTCCTACAAGGCAGGCGGCCTGGCGTCAGTAGTAGGAAGGGATGGCATTGTTGGCTCCAGGGT
GAGGGCCCGACGGGCAGGTGCTTGCCTGGGTAGATGGGGTGTAGATATGTGGCATATAGAGATATATATT
TTATAATGGGAGGGGGGATGGCACCTCCTGGGACTGGTAGAGCTGGGAGGTGTGTCGGCAAGCACATGGC
CCCAAGTCCACGTCCCTAGAAAGATGGGGCCTACAGTGATAGGCGCTCTATAAATACATAGATAGGGTCA
TGTTTAAGAAATATTATGGGGTGGGGAGTGGGGGAAGCAGGCTTAGTGTGCTGAGCCTCGGTCCCCACTG
CAGACCTAAACCCCACACCAATCAGTCAATCTCACACGCATACACAGGAGCATATTTCTGTACTGATGCA
TAATGAATGCACTCACACACACACACACACGCCAGGGGGCTCCCAGCACTCTTCTCCCAGGGTTCAGGAC
TTTGTGGAGTCAGCCCGTGCAGCTATTTACCTCTGTTGCCCTGATTGCAGGTAGACTGTCAATCCCAGAA
GTTGCTAGGTGCTGAGGCAGGAAAAGGAGGAGGTTTCTTTTAGCAGGTGCTCTCCTCTAGCAGGTAGTGT
TCTATCTCTTTTGCCCCTGGTGATGTCAAAAGATTCTTCTGTTAGCAACCTGTGATACTGACCTGCAACT
CTAGGCACCAGTCAAGTCAGCCCTCAACTCCCCCTACCTATTCTCCCCAACTGAAATCAAATTGACATCC
AGGTCTGTCTTGGCCAGGGGGCTTCCATTGGGGAAAAAAAATGGGACAGTGAACCTCACCTATTGAATAA
ACCAAATTAGTAATTAGTGACAAAACCAAACCAAGGCTTTGGACCCCCTGCTCATTACTCAGGAGTAAAT
TCACACAGCTGGTGGCCGGTCTCACCCCTTTTTGGGGTGCTTGGTCCTGGACCCCAATCTACTGTCTCCT
CGACAGATTGGTGTTTAAGATCATGCCCAGTTGCTTCCTGTCACCTCTTGACAGGTGGTGACTGGGCTGC
TGTGGGGCTGGAGCAGCGAGGAAGGGAGCCTCTGCCCCCTCAGTGGAGTCCCTGTGCTAACTGTGGTGCT
CACCCAGCTCCTCCTTGCTCCCGCGGGGCTCTGTCCTTCGGGCCTGGAGTCCACCAGCGCCACTCCAGGA
GGGCTGGGGGAGGAGGGGCTGCCTCAGGCAGTCCAAGGTGCTCCCTCCCCCGCTGGCCAATGCTAAGGTC
TTCCCGGCTCCCAGGGTCAGGACAGAGTCTTCTAAGGCTCAGGCTCTCCCAAGCCACTGCTTAGTGACAG
TTAGCCTTTCCCAGCGGCCCAAGCTCCACCGGAACTGGGCCTGGTGCTCCAACCAAAGGGTTGGTGTTGG
GACTTGGGAAGAGACTTGGTAGCCAAGGGCATGGCAAGCCTGGGGGATTCCTGTGGGCCGGCTTGCTCGA
GGAGGGAGTGCTGGGCACAGAGGAGCCCTTATGGCTGACAGCTTGAGAGCACTTGTTCCTCCAGATCTAG
CCACTTGACCAGGCCCTAGAATGGACCAGATGCCCCAGGGGTCATGGCCCCCCCCCCCCCCCCCTTGCAG
GGACGAGAGTTAACAATCACCAGCTTTTCTTCCGGCCCAGCCTGTGTGCTTGGTCTCACAGTAGAATTTA
GGAGAGTGTGGGGCTTTGGGATTGTGCCAACTTGGCCAAGCTGAGATATGCTTAATCCACCTCAACGCTG
GGGCCTCTGGGGACAGGTATTGGGTCACCTTCCAAGGGGTGCTGAACTTCAGCAGGCTGTAGATAAGGCA
GAGGTTAGAGGCAGTGGAGTGGGGGAACCAAGAAGGCCTAGGGAAAATTGCCAGGCATATTCCCTGTGGA
ATTTTCTTTCCGAAGAGTAGGCCCATTTCCCCCTTAGAAACAAGGGTTCTGGGGGCTGATGACAGCTTCC
CCTGCAGGTGCCAGGTGGCGCATGATGCCAAAGAGATCCGTGGCTCTTGCCTGGGGGAGGTAGCCAGGTG
CTCGTGCTGGGGACACAGTCCTTGGCCTCCTGCCTAAGCAGGAAGCTAGTTTCCAGCAGGCGGCTGGTAC
TAACCCCCCGCCCCACGGGAGCGACAGGCTGGTGCACCGGCAGACTGGGGGGCCGCAGGCGGGCACTAGC
AGGGGTCTGCAGTCCATAGATGATGACATGAATCTGTTGCTGTCCTTTTGTCTCTAGGCCCCAGAGACTT
CTTGCTATTGAACATCCTATTGGAAAGCAAACTTTCCAAGAGAGCACCCGGGACCTGTTAGCTTTTGAAA
CAGGCCTGGGTGTGATTGACTGGTAGAGATGCCTGTGTGATTTTTGGCGCATTGCAGGAGTGTGGATTGG
GACAGGCACCCCAGCAGGAAGGAAGGCTGACACCAAAGGGGTCTCACAGGGCCCAGCTCCCCTTGCTTCC
CGATAATTGGCCACCAGGCCATGGCGACTCTGGGGAAGCCTAGATGGGAACACCCACCTTCTCCAGCAGT
AGAAGGAATATTTCAGCAGAGCCATCTGGGAGATGTTCTTATAGGAAAACTGCAGTCATTCATTTCATAG
TTTTGCTTATATAAATAGAGTACATTTGGGAGGCAGTTTTGAATTCCTGTTGCTACTCTGCTTGGTGCGG
AGTTGCAGTGGCCCCAAGTGTCTTTCAGAGCTGTTGCTTTTAGGATCCCCCTCAAGCTGTGGGTTAGGGC
CAGGGCAGGGAGGGGGTGCTGCCTGGCACTGTGCCCTGAAGAGGGCTGCTTGCTTCCTTTGGTCGGGAGC
ACGGCAGAGCCAACATTCTGGTGGGAACCGAGTTTCTAGCCAGGCCCCTGTTTTGGTCCCGGGAGGTGAG
TAATCTGAGTTTGTCATTGCGTATTCTCCACGCTGCCTGCTCTCAGTGCTGGAGACTAGAGAGCGACCCT
GGAGTTGTCCTCTGGGAGTCCTGCCTGTTTGCCAGGGAGCAGCTGAAACCTCCCCGCTCCCAATCCCGGG
GGTGGAAAAGGTGTAGTGGAGGAATGGTCTGGAGCTGGGATGGCCCTTCTGGCCATGCCCTGCTGAGCCA
GGATTAAGGAAAGTGTCAGCCGCCTCCTTACCAGGCCCTGTGCTGGGGTCAGGAGGACCGGGAGGAGAGG
GAAGGGGTGAGAGGATGGACTCTGCTGTTGTAGTGTGAGCTCTGGAATCTTTACGTTTTGGTAAAGGCAG
GCCAGGGTCTTTGAGGATCGAGTTGGGGAACCTTGCCTCTGTGTTTCCGAGACCCAGCTAAGGTGGGCCT
TTGGTGGGTGACCTGTCTCTGACCCGAGGCAGCAGAGTTTTGGGGGAGGGAGAAACAAGCAAGGAGCAGG
GAGGACAAGGACACAAGATCCCTTTGCTGGTGGTGACTTACTCACTTCCCCCACCAGAGGGCCTGGGACT
CCCGAAACTCCCGTAACTGGAAAGCTCCCTGGCTCAGCTCCACTGGGGGGTGAGGAGGGCGGCTCCCTCA
GCCCCAGCCTAGCGGCCTCCAACCCTGTGCCTGAGAACATGAGCAGCGGTCTAAAGCATCCCCTAGTCCC
ATTTCACTTCACAGACCGATTTGGTTGCACAGCCCTAGAATTCAACAGACAACAAAAATGCTGAAATTCC
TTAGAATACGCAGAGGGAGGAGGTGACTCACCAAGGTGCTAAAACATTTTATTAAAAATATATATTGTTA
AATTAAGTCTGCTGTCTGGACAATGACTGTTTGTTTTGTTTTCTTGTAGTGGAGTTTAAAAGAGACTATT
ATTTTACTCTGATATATTATTATTAAAAAGGCATTTTAACTTTGTACTTGAAAACTAAATGAGCGATTTC
ATGTGTTTTAGCTGAGGCATTGAAGTAGCGGGTGCTTACTTATTTGTGGGAAATCATGTATTCATACTGA
AGAATAAACTCCGTAGCTGATTTGGTAATAACTTTTACTGTTACAGACGATTTCGCTTTGACCCCGGTGG
CAGAGCACTTTTACTCCACATTCCACGCAGCTAAATGCAGGACTTCCCAGACTCTTCCCTCCCCAGCCCT
CTTTCCTTTCTCTTAAGTTCCTCTTTTCTCCTTTATTATTATTATTTTTTTTTCCTGGTTCCAGCATCCT
TTCACCACTGTATTTTTTTTTTTTTTTTTTTTTTTTTTTTTTTTTTTTTTTTTTTTTTTTTTTTAGGGTT
GCTTCTCTATTTATTGACAATAATAATGTACATTTCACAGTCTGGTTCTAGGACATTCAGGGAAGGGCGA
GTGGGGGCCCGAGGGGTCCCTGCCCCACCCGCCTGGTGTCCTGTTGTATCTCTGTGTAAGTGATGCTCGT
GACATAGCTGTTTATGAAATAATTAAAGAGGTCAATGCGTACAACAGATGTAGAAAGTCTGTCAGTTCCG
CCTTCATCACATTTTTTTTTGTGCCCAGAAGAATATAATAAAGCTCCTTTCTAATGTACTTGTGCTGGAG
AACACTTGAATAAATGGACTGTTTTTGTGCAAAAAGAAAAA
SEQ ID NO: 32 (NM_153759.3, DNMT3A isoform b mRNA):
CCCTCCGCCCCCAGCCCCATCGCCCCCTTCCCCTCCCCCAAGACGGGCAGCTACTTCCAGAGCTTCAGGG
CCGCGGCTCACACCTGAGCGCGACTGCAGAGGGGCTGCACCTGGCCTTATGGGGATCCTGGAGCGGGTTG
TGAGAAGGAATGGGCGCGTGGATCGTAGCCTGAAAGACGAGTGTGATACGGCTGAGAAGAAAGCCAAGGT
CATTGCAGGAATGAATGCTGTGGAAGAAAACCAGGGGCCCGGGGAGTCTCAGAAGGTGGAGGAGGCCAGC
CCTCCTGCTGTGCAGCAGCCCACTGACCCCGCATCCCCCACTGTGGCTACCACGCCTGAGCCCGTGGGGT
CCGATGCTGGGGACAAGAATGCCACCAAAGCAGGCGATGACGAGCCAGAGTACGAGGACGGCCGGGGCTT
TGGCATTGGGGAGCTGGTGTGGGGGAAACTGCGGGGCTTCTCCTGGTGGCCAGGCCGCATTGTGTCTTGG
TGGATGACGGGCCGGAGCCGAGCAGCTGAAGGCACCCGCTGGGTCATGTGGTTCGGAGACGGCAAATTCT
CAGTGGTGTGTGTTGAGAAGCTGATGCCGCTGAGCTCGTTTTGCAGTGCGTTCCACCAGGCCACGTACAA
CAAGCAGCCCATGTACCGCAAAGCCATCTACGAGGTCCTGCAGGTGGCCAGCAGCCGCGCGGGGAAGCTG
TTCCCGGTGTGCCACGACAGCGATGAGAGTGACACTGCCAAGGCCGTGGAGGTGCAGAACAAGCCCATGA
TTGAATGGGCCCTGGGGGGCTTCCAGCCTTCTGGCCCTAAGGGCCTGGAGCCACCAGAAGAAGAGAAGAA
TCCCTACAAAGAAGTGTACACGGACATGTGGGTGGAACCTGAGGCAGCTGCCTACGCACCACCTCCACCA
GCCAAAAAGCCCCGGAAGAGCACAGCGGAGAAGCCCAAGGTCAAGGAGATTATTGATGAGCGCACAAGAG
AGCGGCTGGTGTACGAGGTGCGGCAGAAGTGCCGGAACATTGAGGACATCTGCATCTCCTGTGGGAGCCT
CAATGTTACCCTGGAACACCCCCTCTTCGTTGGAGGAATGTGCCAAAACTGCAAGAACTGCTTTCTGGAG
TGTGCGTACCAGTACGACGACGACGGCTACCAGTCCTACTGCACCATCTGCTGTGGGGGCCGTGAGGTGC
TCATGTGCGGAAACAACAACTGCTGCAGGTGCTTTTGCGTGGAGTGTGTGGACCTCTTGGTGGGGCCGGG
GGCTGCCCAGGCAGCCATTAAGGAAGACCCCTGGAACTGCTACATGTGCGGGCACAAGGGTACCTACGGG
CTGCTGCGGCGGCGAGAGGACTGGCCCTCCCGGCTCCAGATGTTCTTCGCTAATAACCACGACCAGGAAT
TTGACCCTCCAAAGGTTTACCCACCTGTCCCAGCTGAGAAGAGGAAGCCCATCCGGGTGCTGTCTCTCTT
TGATGGAATCGCTACAGGGCTCCTGGTGCTGAAGGACTTGGGCATTCAGGTGGACCGCTACATTGCCTCG
GAGGTGTGTGAGGACTCCATCACGGTGGGCATGGTGCGGCACCAGGGGAAGATCATGTACGTCGGGGACG
TCCGCAGCGTCACACAGAAGCATATCCAGGAGTGGGGCCCATTCGATCTGGTGATTGGGGGCAGTCCCTG
CAATGACCTCTCCATCGTCAACCCTGCTCGCAAGGGCCTCTACGAGGGCACTGGCCGGCTCTTCTTTGAG
TTCTACCGCCTCCTGCATGATGCGCGGCCCAAGGAGGGAGATGATCGCCCCTTCTTCTGGCTCTTTGAGA
ATGTGGTGGCCATGGGCGTTAGTGACAAGAGGGACATCTCGCGATTTCTCGAGTCCAACCCTGTGATGAT
TGATGCCAAAGAAGTGTCAGCTGCACACAGGGCCCGCTACTTCTGGGGTAACCTTCCCGGTATGAACAGG
CCGTTGGCATCCACTGTGAATGATAAGCTGGAGCTGCAGGAGTGTCTGGAGCATGGCAGGATAGCCAAGT
TCAGCAAAGTGAGGACCATTACTACGAGGTCAAACTCCATAAAGCAGGGCAAAGACCAGCATTTTCCTGT
CTTCATGAATGAGAAAGAGGACATCTTATGGTGCACTGAAATGGAAAGGGTATTTGGTTTCCCAGTCCAC
TATACTGACGTCTCCAACATGAGCCGCTTGGCGAGGCAGAGACTGCTGGGCCGGTCATGGAGCGTGCCAG
TCATCCGCCACCTCTTCGCTCCGCTGAAGGAGTATTTTGCGTGTGTGTAAGGGACATGGGGGCAAACTGA
GGTAGCGACACAAAGTTAAACAAACAAACAAAAAACACAAAACATAATAAAACACCAAGAACATGAGGAT
GGAGAGAAGTATCAGCACCCAGAAGAGAAAAAGGAATTTAAAACAAAAACCACAGAGGCGGAAATACCGG
AGGGCTTTGCCTTGCGAAAAGGGTTGGACATCATCTCCTGATTTTTCAATGTTATTCTTCAGTCCTATTT
AAAAACAAAACCAAGCTCCCTTCCCTTCCTCCCCCTTCCCTTTTTTTTCGGTCAGACCTTTTATTTTCTA
CTCTTTTCAGAGGGGTTTTCTGTTTGTTTGGGTTTTGTTTCTTGCTGTGACTGAAACAAGAAGGTTATTG
CAGCAAAAATCAGTAACAAAAAATAGTAACAATACCTTGCAGAGGAAAGGTGGGAGAGAGGAAAAAAGGA
AATTCTATAGAAATCTATATATTGGGTTGTTTTTTTTTTTGTTTTTTGTTTTTTTTTTTTGGGTTTTTTT
TTTTACTATATATCTTTTTTTTGTTGTCTCTAGCCTGATCAGATAGGAGCACAAGCAGGGGACGGAAAGA
GAGAGACACTCAGGCGGCAGCATTCCCTCCCAGCCACTGAGCTGTCGTGCCAGCACCATTCCTGGTCACG
CAAAACAGAACCCAGTTAGCAGCAGGGAGACGAGAACACCACACAAGACATTTTTCTACAGTATTTCAGG
TGCCTACCACACAGGAAACCTTGAAGAAAATCAGTTTCTAGAAGCCGCTGTTACCTCTTGTTTACAGTTT
ATATATATATGATAGATATGAGATATATATATAAAAGGTACTGTTAACTACTGTACAACCCGACTTCATA
ATGGTGCTTTCAAACAGCGAGATGAGTAAAAACATCAGCTTCCACGTTGCCTTCTGCGCAAAGGGTTTCA
CCAAGGATGGAGAAAGGGAGACAGCTTGCAGATGGCGCGTTCTCACGGTGGGCTCTTCCCCTTGGTTTGT
AACGAAGTGAAGGAGGAGAACTTGGGAGCCAGGTTCTCCCTGCCAAAAAGGGGGCTAGATGAGGTGGTCG
GGCCCGTGGACAGCTGAGAGTGGGATTCATCCAGACTCATGCAATAACCCTTTGATTGTTTTCTAAAAGG
AGACTCCCTCGGCAAGATGGCAGAGGGTACGGAGTCTTCAGGCCCAGTTTCTCACTTTAGCCAATTCGAG
GGCTCCTTGTGGTGGGATCAGAACTAATCCAGAGTGTGGGAAAGTGACAGTCAAAACCCCACCTGGAGCA
AATAAAAAAACATACAAAACGTACTGGTGCTTTCCTGT
SEQ ID NO: 33 (NM_175629.2, DNMT3A isoform a mRNA):
GCAGTGGGCTCTGGCGGAGGTCGGGAGAACTGCAGGGCGAAGGCCGCCGGGGGCTCCGCGGGCTGCGGGG
GGAGGCACTTGACACCGGCCCGGGGAGAGGAGGGGCCGCTGTCCCTGCGGCCAGTGCTGGATGCGGGGAC
CCAGCGCAGAAGCAGCGCCAGGTGGAGCCATCGAAGCCCCCACCCACAGGCTGACAGAGGCACCGTTCAC
CAGAGGGCTCAACACCGGGATCTATGTTTAAGTTTTAACTCTCGCCTCCAAAGACCACGATAATTCCTTC
CCCAAAGCCCAGCAGCCCCCCAGCCCCGCGCAGCCCCAGCCTGCCTCCCGGCGCCCAGATGCCCGCCATG
CCCTCCAGCGGCCCCGGGGACACCAGCAGCTCTGCTGCGGAGCGGGAGGAGGACCGAAAGGACGGAGAGG
AGCAGGAGGAGCCGCGTGGCAAGGAGGAGCGCCAAGAGCCCAGCACCACGGCACGGAAGGTGGGGCGGCC
TGGGAGGAAGCGCAAGCACCCCCCGGTGGAAAGCGGTGACACGCCAAAGGACCCTGCGGTGATCTCCAAG
TCCCCATCCATGGCCCAGGACTCAGGCGCCTCAGAGCTATTACCCAATGGGGACTTGGAGAAGCGGAGTG
AGCCCCAGCCAGAGGAGGGGAGCCCTGCTGGGGGGCAGAAGGGGGGGGCCCCAGCAGAGGGAGAGGGTGC
AGCTGAGACCCTGCCTGAAGCCTCAAGAGCAGTGGAAAATGGCTGCTGCACCCCCAAGGAGGGCCGAGGA
GCCCCTGCAGAAGCGGGCAAAGAACAGAAGGAGACCAACATCGAATCCATGAAAATGGAGGGCTCCCGGG
GCCGGCTGCGGGGTGGCTTGGGCTGGGAGTCCAGCCTCCGTCAGCGGCCCATGCCGAGGCTCACCTTCCA
GGCGGGGGACCCCTACTACATCAGCAAGCGCAAGCGGGACGAGTGGCTGGCACGCTGGAAAAGGGAGGCT
GAGAAGAAAGCCAAGGTCATTGCAGGAATGAATGCTGTGGAAGAAAACCAGGGGCCCGGGGAGTCTCAGA
AGGTGGAGGAGGCCAGCCCTCCTGCTGTGCAGCAGCCCACTGACCCCGCATCCCCCACTGTGGCTACCAC
GCCTGAGCCCGTGGGGTCCGATGCTGGGGACAAGAATGCCACCAAAGCAGGCGATGACGAGCCAGAGTAC
GAGGACGGCCGGGGCTTTGGCATTGGGGAGCTGGTGTGGGGGAAACTGCGGGGCTTCTCCTGGTGGCCAG
GCCGCATTGTGTCTTGGTGGATGACGGGCCGGAGCCGAGCAGCTGAAGGCACCCGCTGGGTCATGTGGTT
CGGAGACGGCAAATTCTCAGTGGTGTGTGTTGAGAAGCTGATGCCGCTGAGCTCGTTTTGCAGTGCGTTC
CACCAGGCCACGTACAACAAGCAGCCCATGTACCGCAAAGCCATCTACGAGGTCCTGCAGGTGGCCAGCA
GCCGCGCGGGGAAGCTGTTCCCGGTGTGCCACGACAGCGATGAGAGTGACACTGCCAAGGCCGTGGAGGT
GCAGAACAAGCCCATGATTGAATGGGCCCTGGGGGGCTTCCAGCCTTCTGGCCCTAAGGGCCTGGAGCCA
CCAGAAGAAGAGAAGAATCCCTACAAAGAAGTGTACACGGACATGTGGGTGGAACCTGAGGCAGCTGCCT
ACGCACCACCTCCACCAGCCAAAAAGCCCCGGAAGAGCACAGCGGAGAAGCCCAAGGTCAAGGAGATTAT
TGATGAGCGCACAAGAGAGCGGCTGGTGTACGAGGTGCGGCAGAAGTGCCGGAACATTGAGGACATCTGC
ATCTCCTGTGGGAGCCTCAATGTTACCCTGGAACACCCCCTCTTCGTTGGAGGAATGTGCCAAAACTGCA
AGAACTGCTTTCTGGAGTGTGCGTACCAGTACGACGACGACGGCTACCAGTCCTACTGCACCATCTGCTG
TGGGGGCCGTGAGGTGCTCATGTGCGGAAACAACAACTGCTGCAGGTGCTTTTGCGTGGAGTGTGTGGAC
CTCTTGGTGGGGCCGGGGGCTGCCCAGGCAGCCATTAAGGAAGACCCCTGGAACTGCTACATGTGCGGGC
ACAAGGGTACCTACGGGCTGCTGCGGCGGCGAGAGGACTGGCCCTCCCGGCTCCAGATGTTCTTCGCTAA
TAACCACGACCAGGAATTTGACCCTCCAAAGGTTTACCCACCTGTCCCAGCTGAGAAGAGGAAGCCCATC
CGGGTGCTGTCTCTCTTTGATGGAATCGCTACAGGGCTCCTGGTGCTGAAGGACTTGGGCATTCAGGTGG
ACCGCTACATTGCCTCGGAGGTGTGTGAGGACTCCATCACGGTGGGCATGGTGCGGCACCAGGGGAAGAT
CATGTACGTCGGGGACGTCCGCAGCGTCACACAGAAGCATATCCAGGAGTGGGGCCCATTCGATCTGGTG
ATTGGGGGCAGTCCCTGCAATGACCTCTCCATCGTCAACCCTGCTCGCAAGGGCCTCTACGAGGGCACTG
GCCGGCTCTTCTTTGAGTTCTACCGCCTCCTGCATGATGCGCGGCCCAAGGAGGGAGATGATCGCCCCTT
CTTCTGGCTCTTTGAGAATGTGGTGGCCATGGGCGTTAGTGACAAGAGGGACATCTCGCGATTTCTCGAG
TCCAACCCTGTGATGATTGATGCCAAAGAAGTGTCAGCTGCACACAGGGCCCGCTACTTCTGGGGTAACC
TTCCCGGTATGAACAGGCCGTTGGCATCCACTGTGAATGATAAGCTGGAGCTGCAGGAGTGTCTGGAGCA
TGGCAGGATAGCCAAGTTCAGCAAAGTGAGGACCATTACTACGAGGTCAAACTCCATAAAGCAGGGCAAA
GACCAGCATTTTCCTGTCTTCATGAATGAGAAAGAGGACATCTTATGGTGCACTGAAATGGAAAGGGTAT
TTGGTTTCCCAGTCCACTATACTGACGTCTCCAACATGAGCCGCTTGGCGAGGCAGAGACTGCTGGGCCG
GTCATGGAGCGTGCCAGTCATCCGCCACCTCTTCGCTCCGCTGAAGGAGTATTTTGCGTGTGTGTAAGGG
ACATGGGGGCAAACTGAGGTAGCGACACAAAGTTAAACAAACAAACAAAAAACACAAAACATAATAAAAC
ACCAAGAACATGAGGATGGAGAGAAGTATCAGCACCCAGAAGAGAAAAAGGAATTTAAAACAAAAACCAC
AGAGGCGGAAATACCGGAGGGCTTTGCCTTGCGAAAAGGGTTGGACATCATCTCCTGATTTTTCAATGTT
ATTCTTCAGTCCTATTTAAAAACAAAACCAAGCTCCCTTCCCTTCCTCCCCCTTCCCTTTTTTTTCGGTC
AGACCTTTTATTTTCTACTCTTTTCAGAGGGGTTTTCTGTTTGTTTGGGTTTTGTTTCTTGCTGTGACTG
AAACAAGAAGGTTATTGCAGCAAAAATCAGTAACAAAAAATAGTAACAATACCTTGCAGAGGAAAGGTGG
GAGAGAGGAAAAAAGGAAATTCTATAGAAATCTATATATTGGGTTGTTTTTTTTTTTGTTTTTTGTTTTT
TTTTTTTGGGTTTTTTTTTTTACTATATATCTTTTTTTTGTTGTCTCTAGCCTGATCAGATAGGAGCACA
AGCAGGGGACGGAAAGAGAGAGACACTCAGGCGGCAGCATTCCCTCCCAGCCACTGAGCTGTCGTGCCAG
CACCATTCCTGGTCACGCAAAACAGAACCCAGTTAGCAGCAGGGAGACGAGAACACCACACAAGACATTT
TTCTACAGTATTTCAGGTGCCTACCACACAGGAAACCTTGAAGAAAATCAGTTTCTAGAAGCCGCTGTTA
CCTCTTGTTTACAGTTTATATATATATGATAGATATGAGATATATATATAAAAGGTACTGTTAACTACTG
TACAACCCGACTTCATAATGGTGCTTTCAAACAGCGAGATGAGTAAAAACATCAGCTTCCACGTTGCCTT
CTGCGCAAAGGGTTTCACCAAGGATGGAGAAAGGGAGACAGCTTGCAGATGGCGCGTTCTCACGGTGGGC
TCTTCCCCTTGGTTTGTAACGAAGTGAAGGAGGAGAACTTGGGAGCCAGGTTCTCCCTGCCAAAAAGGGG
GCTAGATGAGGTGGTCGGGCCCGTGGACAGCTGAGAGTGGGATTCATCCAGACTCATGCAATAACCCTTT
GATTGTTTTCTAAAAGGAGACTCCCTCGGCAAGATGGCAGAGGGTACGGAGTCTTCAGGCCCAGTTTCTC
ACTTTAGCCAATTCGAGGGCTCCTTGTGGTGGGATCAGAACTAATCCAGAGTGTGGGAAAGTGACAGTCA
AAACCCCACCTGGAGCAAATAAAAAAACATACAAAACGTACTGGTGCTTTCCTGT
SEQ ID NO: 34 (NM_175630.1, DNMT3A isoform c mRNA):
GCAGTGGGCTCTGGCGGAGGTCGGGAGAACTGCAGGGCGAAGGCCGCCGGGGGCTCCGCGGGCTGCGGGG
GGAGGCACTTGACACCGGCCCGGGGAGAGGAGGGGCCGCTGTCCCTGCGGCCAGTGCTGGATGCGGGGAC
CCAGCGCAGAAGCAGCGCCAGGTGGAGCCATCGAAGCCCCCACCCACAGGCTGACAGAGGCACCGTTCAC
CAGAGGGCTCAACACCGGGATCTATGTTTAAGTTTTAACTCTCGCCTCCAAAGACCACGATAATTCCTTC
CCCAAAGCCCAGCAGCCCCCCAGCCCCGCGCAGCCCCAGCCTGCCTCCCGGCGCCCAGATGCCCGCCATG
CCCTCCAGCGGCCCCGGGGACACCAGCAGCTCTGCTGCGGAGCGGGAGGAGGACCGAAAGGACGGAGAGG
AGCAGGAGGAGCCGCGTGGCAAGGAGGAGCGCCAAGAGCCCAGCACCACGGCACGGAAGGTGGGGCGGCC
TGGGAGGAAGCGCAAGCACCCCCCGGTGGAAAGCGGTGACACGCCAAAGGACCCTGCGGTGATCTCCAAG
TCCCCATCCATGGCCCAGGACTCAGGCGCCTCAGAGCTATTACCCAATGGGGACTTGGAGAAGCGGAGTG
AGCCCCAGCCAGAGGAGGGGAGCCCTGCTGGGGGGCAGAAGGGCGGGGCCCCAGCAGAGGGAGAGGGTGC
AGCTGAGACCCTGCCTGAAGCCTCAAGAGCAGTGGAAAATGGCTGCTGCACCCCCAAGGAGGGCCGAGGA
GCCCCTGCAGAAGCGGGTGAGTCCTCAGCACCAGGGGCAGCCTCTTCTGGGCCCACCAGCATACCCTGAG
AGTCAGGGACTTGGCTCTCCAGCAGGTCCCAGGAAGGATGGTCTGGGTCGTGGCTAAAGGTCTGCTTGCC
AAGGCTATGGCCTGGAGGCTACTGGCTGGATGCAGCCTGCGCATATGTTTTATTTGGCCCATAGAGTGTT
TTAAACATTTAAAAAATTAGTTGCCAGTATTTAAAAATCAAAAAATTTCACATAAAAATCTGGAGTTTTG
GCTTCTCATGAAAAAAAAAAAAGCTAGATCTGGCAACAGCGGGCTTTCATAACGCCAACGATTGCTAGAC
TGGGATAATGGCGGTCCCTCCATCGCCTTCTGTGGCTGGTTGTGGGCCTTAGTTTTCTGCAGCTCTACCT
GGCCTGCTTACTCTCCCACGTGCCATGCAGTTCCTGGGGGTTGCTGTATTTGTAGCCCCTGGCCTGGGCA
CTCAAGGGCAGCAGATACCCTGTTTGCCTCCCTGAGTGCAGAGGTCCTGAGCCCACCCTAGTTGGGCTGA
CTCAACTGGAAATTTGGTTGTGACAGTGGCGTGGGGAGAGGGCTGGGTGATTGTATTCTGTGTACTGCCC
AGCCCAGGCCTCTTCATCTGGGGACTTTTTGGCCTAACCCTGGAAGCCTGGAAAGTTGCCCACTTTTCTC
TTTCAGGTTAAGCCAGCAATTTCAGGGCCAACCGAGCTGTAAACATGTTAGTAATGAGGACAACTAGCAT
TTGTACAGGGCTTCACAGTTTACAAAGCGCTTTCTCATACATTATCACATTTGATCCTCCCAGGGCCCTG
CCAGGTTGTTTTGCATATGTGCATTTTAATTTCAAAAAGTCTTCCTTCCAAGCGTGTATGATGAAATGAG
TAAATTGATTAATTGGCGTAACTTATTTTGCATGGATCCAACCTAATGTTCATGCAGGATAGAGAACATT
TCCAGAATACAAATTTCCAAACTTAAAAAAAAAAAAAAAAAAAAAAAAAAAAAAAAAA
SEQ ID NO: 35 (NC_000002.12: c208255071-208236227 Homo sapiens chromosome 2,
GRCh38.p13 Primary Assembly, IDH1 genomic sequence):
GGGTTTCTGCAGAGTCTACTTCAGAAGCGGAGGCACTGGGAGTCCGGTTTGGGATTGCCAGGCTGTGGTT
GTGAGTCTGAGCTTGTGAGCGGCTGTGGCGCCCCAACTCTTCGCCAGCATATCATCCCGGCAGGTAACCT
CAGGCTCCAAGGGGCGGCCCCGGTCCCTGGCTGTGGAGGGGTGGCTCTAATTCCGCAGAAGGCAGGAATG
GGGTAAAGGAAAAAAGCGCAGATCTTGGGTGTGGAGGGAGTCAAGGCCTGCGTGCAAAGAAGGGAACAAA
TCCAGGTATTGAGAATGGGAAAAGAAAAGGGAAGGAGGGGATGCTGGATGCTTGGATATGCAGAAGGTCT
GCTTTTGGGCCTGGGAGGGAGAAACTGGGCGGTGAGGAAGTTGGATGCAACGACAAGGGGAAAATCAGGT
CTGCACTTGGGTTGGAAAATCGAGGGGTGGAAGTTGCAAGAATGCATGTAGAAGACTAAGCCTGGAGTGT
AGGAGGAAGAAAACGTGCAGCAGCTGGGGGCTGGATGCAAAGAAGGCTGTAAGCCGCAGACGGGCGTCTG
CAGGCAACTCAGGCGGCGAGGAATAGCCTTGAAGCCGATCCCTCGCGTCCGCCAGGGGGCGTGGAGGGGC
GGAGCTCCAGCGGCCTTCAGAGGGGTCGCTAGGCCACACGGGGCGAGAGGGGGCTGGGGGAAACACCCCC
GCCAGTTCCGGGTGCCTGGAGTTTAAAAGGTCCCAGCACCAGCGAGGGGAGGGGAGGGGAGGGGCGGGCG
TGGAGACCTGGAAGGAGGTACCGATCCAGCCTTGATTGCAGCCCTCTTGCACAACGGCCATAAGGACGGG
TGCAGGCCAAGAGCAAGGACATGCAAACCCCAAGACCTGCTAACGGGCGGCGAAGCGGGGGCACGCCCTC
GCACACGCAGAGATAAATTGTGCTCCCATGACCTTTATTTGGAAAGTGCCTGCGGGCCTAAAATTGGCCT
TTGTCCCACCGAGTACACTCAGCACTGTACTTTAAACCGGATAAACTGGGCTGTCTGGCAGGTACTAAAA
ACAGCTAAATTCTCCTGTGAGTGTTCTGTATGTTAACACTTTTATTCCTTGTTTTGTTTTAGGCGATAAA
CTACATTCAGTTGAGTCTGCAAGACTGGGAGGAACTGGGGTGATAAGAAATCTATTCACTGTCAAGGTGA
GAATTAGCAAATTTTCCCCCTTCTTCTGTACTTTCTTTGATGTGGCAGTACAGTGTTTAACTGGTCTTGG
GGGCAGGCCGAGGCACTGCAAGGGAATGAGTGAGGAATATTTCCAACAACGAATTCCAACAGCTTCCTTG
TTTCTTTTAATACAGTGTCTTATCTGCAAGAGATCCTTTTTGCCCAGAAATTATTGTAGTTCATGAACTC
GGCAAAAAACAATCTGGCCAAAAACTTGAGGTTGATGAAAATAATATTAAATGGCTCTTTTTTTTCATTA
AACTACTCTGTATTGTTATTCCAGGTTTATTTTCTGGAATTGCACAATTATGTTAGGAGATAGGATTCAG
CTACTTCATATGAGCTGGTGCCTGCTGGTCTTGGAACAGCCCTTCCTTCTGAAGTTGGGATTGGGTTGGA
GACAAAATCTGAGGGGCAACAAAGGTGGAGAAGTCAGTCCAGTGGGGCCTCAAGCCCATCCCATGGAAAT
CAGATCCAGAAGAAATACAATTATGTTCCACATGGTAGTAACAATAAAGCTTCACTGTTTTAGTTAAAAG
ATGGTAAAAATTTCATGGCTAAGTAATGTGAGGAAAACCTGAAGCGTCTGAAGTATGCATTATGAGATAA
AGAAAGATTACTGCAGAGAGTACAGTTGCTTTAACTGAGAAGGAAATAGTGAACTAAAAAGATATCAGTC
TTCACATTTTAGGTACAATATTTGACCTCTATTAACTGTATTTGCCATCAAGAATAAATTGCCATGTCAT
GACTGTTGCATCATACTATATAGGGACATGGCCATGGAAGTGTATTATCAGGAACCAGATAAGTTCACTG
CCTCCTTTTTTGCTTTGTTTTAAAACAAGGATAGGTGTGACAGTGGAATCAAACATGAACTGAATCATAT
GCTTTCAGCTGTACTACAAATTATACTTGACATTACAAATGGGAAGTAATTGACTCATCCACACCATTCT
GTTCATTTCTTTTAGTCTAGTTCAGTTCCAGAAGGTTCCCCCAAAGGAATCTCCTAAACACTAGTAAGAA
GAATTCTAGAATTGCGAATTTGGGAAGTGAAAATGGGCCTGGGGATTGGAAATACATCCAGGCTTGTCAA
GAAACAGACGCTTACTGTCTACTAATCATTACATTTTTATTTAAAGACCAAATTACTTTAGCTCAATTTT
TCAAGAAAGACTATTTTAGGCTTCTTATCTAATAATTCTTTAAAGTCAATTCTCTTGACTTTTTTAGTCC
TTAAGAGAATAAAAGCAATTTGCAGCTGTGTTGTTGGATTAAAAAAGAGTTTCGATTAGAGCATCATTAG
AAGGAATAGATAACTTTTAGCAGGCTCAGAGCTTCTTCTGCCTCTAGGGACAAGCTGGGACAGTTTCATT
TCTTCCAAAGCGTCCAACCTTGCACTTTCTGAGAATCAGATTGATTTGTATATGCTACCCCCACACCCTA
TCCTTAAAGTGCTGTCTTCCTAGGACAATGCACAGTTTGTCCTATCCCACTCACTAGCAGCTTACCCTTG
CCTGTGAACAGCCAGAAGAAGATGACAGGGACATGCTTGGGTCCTGGGCAGTGGCCCTACTGTATAGAGA
TTCTTCTAATTGAGAAATCACTTTGTTGTATTTAGCCCCACATCTCCAATGTCTGTGTTATCTCTAGGAA
AACAGTTCCACCAGGAATTCTTCGTCTCGCTTTTAGACTTAAAGTTTCCCTGAATATGGGTCACAGGAAC
CAGCTGAATCTCCTCATGGCTAGTTATTTTTATTAAATAACAAAAACAGCCTCTTAAAGGAGGAGAAATG
CTAGCACTCACCATTCTGGACATCTTGAGAGCCCAGTAAAATGATGAGTTGGACTGAAGTGTTCAATAAA
CATGAGATTGTGGAAATAATCTCATTTCTTAAGACAAACCAGTAGTTGAGAATAATGATTGCATTATGAA
GGGCACGTTATAGGGTCAGGCTCTGCCACGCTCTAGCTAGGTGGCCTTGGGAAAGCTCTTTTTTTTTTAA
CAAAGTAAAATGGGGAAATGATGGCCATTTTTTTGGATTGTTATGAGGGCTAGCTCAGAAACCGCGTGTG
AAACATAACACAATACTTTGTTCAGAGAAGATACTCAATTCTAATGGTAAATAGAATTGCTGTTACTAGC
TTTTTATTCAACTACACATAAATTGAATTGCATGAATTGTTCTTTTAGTAGTCACTACGTTGTTTATATA
AACACTACGTTGTTTATATATTTCACTTTATAATGAAATAAATGACAAGGCATGTATTTTTTTTTTCTTT
TTAGGTTTATTGAAGTCAAAATGTCCAAAAAAATCAGTGGCGGTTCTGTGGTAGAGATGCAAGGAGATGA
AATGACACGAATCATTTGGGAATTGATTAAAGAGAAACTCATTTTTCCCTACGTGGAATTGGATCTACAT
AGGTAAATGAGTTACCCCTCCGTGTAGCAAACTCAGAAAGGATAATCTGGCTGGGCATGGTGGTGCACAC
GTGAAGTCCCAGCTACTTAGGAGGCAGAGGCAGGAGGATCCCTCAAGACCAGGAATTCAAGTCCAGCCTG
GGCAACAGAGTGAGACCCCATCACTTTAAAAAAAAAAGGAAAAAAAAGGGAAGGAGAAACAGGAAAAAAA
AAAAGAATATAATCTGAATTATTTTGAGGTGAAGTTAGCTTTTTTTATATAGATATAGCTGTGTTTTGTG
AATAAAGTTTGTTTTAAGCCTAAATATATTCAGATGAATCTTCTAATTTTAACATACTGCTAGAAAAACT
GGATTTATCATCTAAAAGTCAAGAATTATAAGGGAAAGAATGATTTTCAGATTCTTCTAACCTTCTGTTT
CCTGTGAAGGTTTTATTTGTAATAAAACAGTGGCTAAGAATAGTACAATGTATTGAGAAAAGGGAAATAA
GAGAGGATGTAAGAAACAGTAAACTAAATGCAGTTAACACAATTTTGTCATTTCAAACTCAATTTTCTAT
CACTTTACTTAGTCAATGGACCTGTTTCCTTTTCATGCCTCACCTTTTGCCCTTTTCATACAATTGTAGT
GCAGAAAGAATTCATGATGTTTCTTAGTTGAACCCCTCCTTTCCAGCAAAATTCCCTTAAATCCTGTGTC
CCTTTGCCCTGTGTGTAAAGTGCCTGGGGACAGATGCAAAAGTCTATGTAATTCTCTCATTTTTTTCGCT
GTGTTTTTCATAAACTTGCTTCTGTTCTTTGTACCTTTAGGTAGAGATAGAATTTTTAAAAGGGTAAAGT
GGCTGAAGACTGGCATAGAAAAATATTGAGCAACACGTTTCAGCCAAACTATTGGCAACTCTAAGTGTGT
TTGCCTCAGCCCAGAATTAGTCATCAAAATTTGTCCTAAGGACCTGTTTGAGTGGAGACATTTAATTCAC
ATAATGTCATTAAACATACTTTTTAATTGTTCTAAACCTAAGCTGCTGATGTCTATATTGAAATCATGAT
AAGATATAAATACTTGCAGAAGTCAACCAAACATAAGAAATTAGGCATAAAAACATCTTCCAACTAGGTT
AAAAAAAAAAAGCATCTTAGGAACTCCTGTTTCAGGACTTTACATGACAGTAGGTGAACTGGCAGGGTTC
TTTTCCTTATAATGGCTGCAACTAAAGATGCTTTGAGCCTCTTTAGTGTGGAATCATGAGGAGTTTTAAA
GGGGAGTCACTATGACTAGGAGGAAAAAATTTGGTGGCTGCAGACTAGATAATTTTTGGAGGCTCAAACA
AGTTTTAAATTTTTCCAAAATTCCTATTATTATCACTGACTGGTTCTTTTTCAGGAGAATGGAGCAGGTA
GTTTGTTTTATGTAGGCTGGACAGTGGTCTGGGCAATTTGTTCCTTCCTTTCTTATCTTCTTAGAAATGT
TAAGAAGACAAGCAAAAAATCTAATAATTTGCTGATAGATTCTCCTTATGAGAAATGGTTCCTAGGAACT
TGAAAACTACTGATCATACTTCCTGTTGGAGTTGGGATTGGATCAACATTTCCTATGCAAGAAGATAACA
AAGGACATGACTTTGTTAGATTTATATGAATTATCTAAAGTCGAAGCAGTTGGAAGTTATTTTTATTTGG
AAATATGTCAAGATTACTATCTCTTTGTTTGCATCATGAGTTGGGTAGGAGATCTAAGAAAGGAAGATAA
GGGAACCAAAAAGAAATCTAGAGGCAGGTTTTAAGAGCTGAAAAACCTTTTAAATCCTTGGAGAACAGAA
TAAACAAAACATTATTTTGTTTTCTTGCTTTTTAACAAACTATTGCATGCTTCAAATAAAGCTTTGCAGT
CATTTTCTATGCTCCCTGGACAACTAACATTCTTTCAGCTCTGAATGACCTGCAATCTTTGTGTTGGTGT
ATATTAAGCTATTACTCTCTGGTATGTAGAAAACCATTGATGGATTCCTTTACTTACCCAATAAATTGTA
GGCTTGTTTCCAGAGCAAACACTTGCTCAAGAATTACAGAAGGAATTAGCTGGGCATGGTGGTGCATGCC
TGTAGTCCCCGCTACTGGGGAGGCTGAGGCGGGGGAATCACTTGAACCCAGGAAGGCAGAGGTTGAGTGA
GCCAAGATCGTGCCACTGCACTCCAGCCTGGGCAACAAAACGAGACTCATCTAAAAAAAAAAAAAAAAGG
GAAAGGCTCATTTTATGACTAACTGGCATTAATGAAGTAGTCATTCGCATTTTTGGCATCAACTGTCACA
TCTTTACCTCCCACCACTTCAACGTCAAAATACAAAAAATCCTTTAACCCCTCCCACCCCCTTTTATTTT
TAAAGAGCAATATTTACTAAGGGAAAAAGGTGGTCCTTAGACTTACACTTATTATACTAATTATATGGAG
AGAAACCAGGCCATTATTGCCTCTATCTGGTGAAAAACAGGAGGCAAAGGGCAGAGTTTGATGGTTTCCA
TTCATTTTCCTGATAGACAGCTTCCCATTACAAGAGGAGGGGCAGCCCAGCAGTCTAGCCTCTCTTAGTT
CTCTTTGTAGTTGGCACCCATCTTCTGTGTTTAGGGTGTGCCAGTGCTAAAACTTGGCAGATTTTGGTGT
ACTCAGAGCCTTCGCTTTCTGCATAATGAGCTCTATATGCCATCACTGCAGTTGTAGGTTATAACTATCC
ATTTGTCTGAAAAACTTTGCTTCTAATTTTTCTCTTTCAAGCTATGATTTAGGCATAGAGAATCGTGATG
CCACCAACGACCAAGTCACCAAGGATGCTGCAGAAGCTATAAAGAAGCATAATGTTGGCGTCAAATGTGC
CACTATCACTCCTGATGAGAAGAGGGTTGAGGAGTTCAAGTTGAAACAAATGTGGAAATCACCAAATGGC
ACCATACGAAATATTCTGGGTGGCACGGTCTTCAGAGAAGCCATTATCTGCAAAAATATCCCCCGGCTTG
TGAGTGGATGGGTAAAACCTATCATCATAGGTCGTCATGCTTATGGGGATCAAGTAAGTCATGTTGGCAA
TAATGTGATTTTGCATGTTTTTTTTTTCATGGCCCAGAAATTTCCAACTTGTATGTGTTTTATTCTTATC
TTTTGGTATCTACACCCATTAAGCAAGGTATGAAATTGAGAAATGCATATATGTATAACTGTATATTTAC
ACACATTTAGCTAAAGGCAAATACAAATAAACTTACAAATAGGCGTCCATCTCAACACATTTTTTTTCAA
ACATGCTGTTTTTTTTCCTTTATCCTTTTATTCAGTTATACCATATGATATTGCCATTTTTATGTTGGTA
ATTTCATATGGTTCAACCAGATCTGTGGTTTTCAACACTGGCTGCACAATAGGATCCCCTTACAAGTTTT
TTTGGTGGTTTTGTTTTGCTTTGCTTGATTTGTTTCTTTGTTTTAGTTTCAATGCTTGAGTACCACCCTA
CACAAATTAAAATCTGAATTCCATGGGGTTCAGGCATTTTAAAGCTCCCCAGGTGAATCTAATGTGCAAA
CTTGAGAACCACCAAAGATTGTATTAAACATGATCCCATCATGCATAAAAGAAAAAACTGGCTGGGTACT
ATGGTTCACACCTGTAATCCTAGCACTTTGGAGGCCAATTTGGAAGCACTGCTTGAGGCCAGGAGTTTGA
GACTAGCCTGGACAACATAGCAGAACCCTGTCTCTACAAAAATGAAAAGTATTTCAATAATTATGTCAAT
TGTTCATGGAAGAGCCAGTTTTGTTTATTCATATACAAAGTGAGTAGGCGAAGCTGAGTGGGGTGGCATG
TGCCTATAGTCCCAGCTACTTGGAAGGCTGAGGCCTGAGGATCCCTTAAGCCTAGGAGTTCAAGGTTACA
GCTATGAGCTATGATCACACCACTGCCTACGCAACAGAGCAAGACCCTGTCTCGAAAAAGAAAAAGAAAA
AAAAAACTGACTCGGTAGTTGAAAGCAGCCTTACTAAAGCATTCCTTCCTGCCTGGAAAAAAAGTATTGC
TTCTTTACTTCTGTACCAGTACACTTGTTGCCAAATTAAGCAAAAAAACTGCCAAGTAAACAAAATCAAA
GTTCAGTGTTGAAACTGAGGTTTCAGACTTTCACAGAAAGTAGTTTATGTCTTAAAAGGACCTTAAAAAC
AGGAATCTCTCATGCTGAAATCCAGAGGTTTTAATGTAGCCATCATTGAAAACAGTGTGTTGAGGGTAAA
CAGTTTAGTCCTTGAGGGGTCAGTTAACTTTGTTCCACTCTGTGGCTAAAACTATCCTTTCATCCTGGCC
AAAGCCTTCAGAAATAGCTCCCAGGGGCCCAAGCAAGGGAAGGTAGATGGCAGTGGTGCAAACTCGCTAA
TCTTGTAAAAATCACCCTAGATTCTTTCCAGTTCAGTCGTGCCTTTTTTCTGCGTATATCCTTTTTGTTT
TTTGTTTTTGAGACAGAGTCTCGCTCTGTCGCCCAGGCTGGAGTGCAGTGGTGTGATCTTGGCTCACTGC
AACCTCTGCCTCCCGGGTTCAAGCAATTCTCCTGCCTCAGCCTCTTGAGTAGCTGGGATTACAGGCACAT
GCTACCATGCCCGGCTAATTTTTGTGTTTTTAGTAGAGACGGGGTTTCACCATGTTGGTCAGGCTGGTCT
CAAACTCCTGACCTTGTGATCCGCCCGCCTCCGCCTCCGCCTCCCAAAGTGCTGGGATTACAGGCATGCC
ACCATGCCCGGCCTTCCTCTTTTTTTTTTTTTTTTTAATTATTTTGGCATCAGTCATTTGTAAAAATGGC
CATTCTATTTATTAAAGTCCAGAAAGAGAATTTCAAACCTCAGGTTGATTCATGGACTGTTTAAATATAC
ATAAACCTATACAAAGCATTTATGGTGCTTTGGTACCTCAGTTTCTCCCTTTTCAGAAATAGTAATAAAT
TACCACCCATATTTACTTTATGGATTTTATATGTCAAAAATGTTCTGTGTAAAGTAGGTGTTTAATCTTT
ATTGTTACTTACTGCTCTTTTACCAGTTGTTATTAGTATTTCTCTACACTTATAGATGTGGCCGGCACCT
GCTGGTACCTAACTCTGCAAGTATCTGATTCAATTCATTGAAGAAGATACAATGCAGCAAAGACTTATGG
ATGTCTAGTATATGCCAGGCATATTATTCCACTGAGTAGCTGAGGCCCATGAGGGAGGTGCCTGTCCTTG
AACAGCCCTTTCCTAAAAATCATCATCATCCCATTGTAGCTGCTCCCAGAATGATTTGATTGCTCCATAT
GAACAAGTCCTATAACACTAGTAACAATGATACCATGAGAGACCGAGCCCCGATGGCCTAAGTAAGGACG
AGACCTCAAGTCCTTTGACCCTGGATGCATTATTTCTACTATATCATAGTTCCCTCCTTGTAAGACTGAT
GTAAGAATCAAATGAGATTATGCAAGTTAAGCATTTAAAGCATCTGGCATAGAGTAAGGACAAAGCATTA
GCTACCTCAGTACTACAATTAGTATTGTTATCATCACCCTCTAGGGAGACTTCCCTGACTTCCTTCCATG
GTCACCTCTTTTCCTCTGTCATCCTATAGTTTTTTTTTTTTTGGGGGGGGGGTCTAATACCCTCTTTTGG
CATTTGAAAGCTCTTATATTGCTTTTCACTTATATATGTCTTTATAGGGTTGTAAACTTCCTAAAGGAAA
GTGGCTTATGTTATTCTCTGTCCTCTTCAAATCAGTGCTCAGAAAACTTTCCTATACCTCGTATAGGCAC
CTAATAAATATTTACTGATTTAGGGGAATGTCTGGACCTCTTCATCCCCATCTCCTAAAAAAAAAAAAAA
AAAAAGAAGTTTGACATACTGCCCTATTTGTCTAGGTGTCTTCTAGGTCTATATTATATGAATATTCTCC
TTACAATTCCTGCTAGGGTATTTTTTTTTTCTTTATACCTTTTTCCCCTCTACACAGTACAGAGCAACTG
ATTTTGTTGTTCCTGGGCCTGGAAAAGTAGAGATAACCTACACACCAAGTGACGGAACCCAAAAGGTGAC
ATACCTGGTACATAACTTTGAAGGTATGTATGACTGTAGCATAAGCTTCTTTTTTTTTTAAACAAATGAT
TTAGATAATATTTTTATTGCTTTTTTTGTTAGTTATGTGACTGCCTAAAAATATAATCTGAATCTACAGT
GTTTTTGCAAAACCCGAAACTTGACATAAAGAAGTAATTTGCTTCCATACCTTCCTGTGGCATGAATAAA
GGCACAAAAGGGTTACCCACTTAATTGGAAATGCCTTTAGGCAAAGACAAGGAGATGATTATGCATTTAT
CTTAAAGCACAGAGAGAAGGTTGGCAGAAATCTGCTGTGCATGAATTATATACATTAAATATTAATATTC
TCAAATCACCCAGCATATAATCCTGCCCTTTGCTCTGGTAGGGCTCTTGTATCTGGAAGAGGTTTCCTTA
TATATTTGCTGAAGTTTTCTTCTTGCCCTTCTGGGTATTTATCCTGTGAAAAAGTAGAACATTTGCTTTT
TGCCTTTTCTTTCCCAGCTACCCTGCTACCTTTGATGGCAAATCCCACCCAAAAGTTGTTGAAATAATTA
GTGGGCCCTTTTCCTTCACTATTGAAGTTGTTGAAAGCACTCACACAAACAAAAATACCCAACACTCACC
TACCTTTTGTTTTTCTTGCCTCTTCCAAATAATATCTGTTGGAATCTGGTTCTGCAGGCTTCAGCACCTT
TCACCCTGAACCCAAGTTAACCCTGAGCATTGGCATGGATTCAAGTTCTATCTTTAAGTAATAACAAAGT
TCAATAATAGTTGTAAAACACAGTGATTACATGTAGCATAATTATTTCTACTTATTTCTACCAACTACAG
TAGGAGTGCTCAGTATCCTCCAGGAGATTATAAGAATTTTCATTCAGTATTTGAAATTTAAGTTTTTCTC
AATAACTGAGGATTGAGTACACCCCTAAAGTTTAAAATAGATGTTGGGAATTGTAAAGGTAGGGTGTTTG
TCCATTCTTGCGTTGCTATAAAGAAATACCTGAGGCTGCGTAAGTTATAAAGAAAAGAGGTTTAATTGGC
TCATGGTTCTGCAGGCTGTACAAGCCTGGCTCCAACACCTGTTCCTGGTGAGGGCCTCAGGAAGCTTCCA
ATCTTGGCAGAAGGCAAACAGACAATAGGTGTATCACATGGCAAGAGCGGGAACAAGAGAAAGAGAAGGG
GGAGGTCCCAGACTCTTAAACAACCAGATTTCAAGTGAATTAACTGAGCGAGAACTCACGTAATCACCAA
AGGGGATGGTCCTAAGCCATTCATAAGGGATCCAACCCCATGTTCCAATCTCCTCCCACTGGGCCCCACC
TCCAACACTGGGAATCACATTTCAACATGAGATTTGGAAGGGACAAATATCCAAACCATATCAGTATGTA
AAAGAAGTTTCAAAATTATTTGCCTTTTCTTAAATGTATTTCTGGTTCATATCAGTTTTTGGCATTTCTG
CATCTTCAGGATTTTAAAGACTGTATGTTTTTTCATTTCTGCTGTCCTTCCAAAACAAGTTAGAATGTCC
TTATCTAGAACTGGCTTTTCTCTCATTATGTGCCTCAGAAATTTGTTGGAAACCTGTCTGGGACTTCTGA
AATCTAGTCTCAGATGTGAGAGCTGAGAAGTAGGTCATTTGGTTGTGGTGGGTGATTTTAGCCTTATTAC
AACTACATTACATTTATTCTTCTCCCTTTTTTCCTCTCACTGGCTTCTCCTCTACAGAAGGTGGTGGTGT
TGCCATGGGGATGTATAATCAAGATAAGTCAATTGAAGATTTTGCACACAGTTCCTTCCAAATGGCTCTG
TCTAAGGGTTGGCCTTTGTATCTGAGCACCAAAAACACTATTCTGAAGAAATATGATGGGCGTTTTAAAG
ACATCTTTCAGGAGATATATGACAAGTAACTATAGTTCTTTTTTAACTTTTTCTTTATTCTCAAGTAAGC
TGACATTTGCATATAGAGTATGTATCTCCCTATCCCTATGATTCTGGGTAAATTGAGTGAAAATCATCAC
TGTCTCTTAAAGATAAGGGCAAAACTGAGGTTTAGCAGGAGTGAAACAAAACCACTCTGACTTGCTTTTA
TAAGATAAACATGTTCTTAAATAAGGTCCCCACATGAATTGTTTTCAGAGCCAGGTTCTTTAATGTTTAA
ATAAACGTACGCTGTTAGTGATAAGTTCTAATTTTTCATGTGTGAGTTTCTAAGTCAGACCCATTTCCAT
TCTTTCTGAAGCCCATTGTTAGAATTTAAAATTCTGTTGGCCAGGCACGGTGGCTCATGCCTGTAATCCC
AGCACTTTGGGAGGCCGAGGCGGGCGGATCACGAGGTCAGGAGATCGAGACCATCCTGGCTAACACGGTG
AAACCCTGACTCTACTAAAAATACCAAAAATTAGCCGGGCGTGGTGGCGGGCGCCTCTAGTCCCAGTTAC
TGGGGAGGCTGAGGCAGGAGAATGGCGTGAACCCGGGAGGCGGAGGTTGCAGTGAGCCGAAATCACGCCA
CTGCACTGCAGCCTGGGCGACAGAGCGAGACTCCGTCTCAAAAAAAAAAAAAGAAAAGAAAAGAAAAAAA
AGAATTTAAAATTCTGTTTTAGTGGAGTCATTTGAACTTAAGTCTAAGTTTATAACAACACTGGCTTCCA
CAGCACAGGAGGTGAGCATGTGTTAATATTTAAGATTGGCATAACTCCCTTTAGGTGCAAGTGTTCAGGC
CAAAATGTTCCTGAGGCATTTTGATTCCTCCTCCTGCTGCCCATCTATACCAAGCCCAGAAACTGTCTGG
AATATATTTTAGTTTCCTGAATGACACCAAGAAGTAGAACAGTCTTTTCAAAAATGTATTTTAAAAATAA
GCTGAATCTCAAGAATCTGATCTATAGTATAATGAAAACTGAAAAGTGAAGTAGTCATTGGGATACTCTA
CTGTCTCACTTAATTCTCACGGCTTCCCTGCAAGGTGGGTAAAATTGTTCCTACAGATAGTCAAATTGAG
TTTTACAGTTAGAAAATGATTGGGCTAGGATTTGAGCCAATGTCTGTCAGATTCCTGAGTTTCTGCTACT
TCTACTAAAATATGCTGCTTCTTGTGTGTCCGGTCTTCTGTTTGGGACAAGCAGATGATATCCCTAACAA
AATCAATTTTCTTTATTATTATTCTCTTTTACCTTTTGTTTCCCAGGCAGTACAAGTCCCAGTTTGAAGC
TCAAAAGATCTGGTATGAGCATAGGCTCATCGACGACATGGTGGCCCAAGCTATGAAATCAGAGGGAGGC
TTCATCTGGGCCTGTAAAAACTATGATGGTGACGTGCAGTCGGACTCTGTGGCCCAAGGTACAGAGGTGG
AGCCTGGCCTGTAGGAACAGGGTCATTCCAGGGTAGTTCTCTAATTTGGGAAGGGGAGTTTTGTAGTCCT
TTGTTGTAAAATCTTGGAATGTTAAACCTCCAGGTCCACCGTCTTACCTGACAGCTAAGGAAATTAAGCT
CAGACTAGAGAAATGATTTACTCAAGACCACATGGGAAGTAAATTAGTGGATTGACACTCCTGGCTCCAG
TGTCACTCCTTGAAGTTTTAAAGCCTACCTGATCTGAAAATCACTCCCTTTCCCTGCCCTCCTTGTCCTT
TGGTGAAAATGTAAAAAACCAGGGCACTCAGGCAAGGAGGGATAAAGTGAGGAGCTTGATCATGTCATAA
CCAAGCTTTGGTGCCTGCAAAGCTCATTTATTGGGCAGCAGGACCTACTCATGTCCCTCTGCCAGCTGCT
GTTCTGGTAGAATATAAAACAAATGTGTTTGGCTGGGCACAGTGGCTCACGCCTGTAATCCTAGCACTTT
GAGAGGCCAAGGCAGTGGATCACTTGAGGCCAGGAGTTCAAGACCAGCCTGGCCAACATAGCAAAACCCC
ATCTCTACTTCAAAAAAAAAAAAAAAAAAAAATTAGCCAGGTGTGGTCGCATGCCTATAGTCCCTGGCAC
TCAGGAGGCTAAAGCATGAGAATTTCTTGAGCCTGGGAAGCGGAGGTTGCAGTGAGCTGAGATCACGCCA
CTATACTCCAGCCTGGGCAACAGAGCAAGACTCTTGTCTCAACAACAACAAAGAAAAAGTGTTTTTGTTT
ACTGCTTAGCTTACACACAAATCAATTGAGCAAAGAACCCTAGGGGCTGTTTACAGAAATTCCAGGAGAA
GTTTCATGCATGGGGTAAGGGCCAAATCAACAGAAACACAGCGCCTGGTTTCCTCAGCACCAAGTTGCCC
TTCAGTTTCAGTTGCCCTTGGTAGCTGTATGAACTTGAATCTCCCCAGATGAATGTTTGTTAACTTTAAT
TATGTTTGTTTTTCCCAAGAAGTACTACTTTTTCTGTCTCTTATCAGTGTTAAACATCTCCTCTCAGTTT
GAAAATCAGTAGATCAACCTGGTTAAAGATAACTTTTTGCCAGATAAATGAGTTACACAACTCTCCTTAC
CATCCTTCTGTTTCTCTACATCCTCACAATCATTTGTACTGAATGCAACAGAGGAAGGAGTGTTATTGCT
TTGTGATGACTCCGTGGTTGATTATTAACTTCACAGGTGATGTTGGGAGGTAGCAAAAATGGAGAAAAGA
AGAAAATCAATTATATAGATATAAGAGTTGTAATGTGGTTCTTTCAATAAGAAACCACAGAAGAGAACTG
TTGTACTGAGGGTATGAAAGAGAGCGAGGAAGGGCAACTGGGATGTTCTTAGATCCAACCGATCCTGTGT
TTACCTGCCTTCTCTTCCCAGTCCCAGCAAGTGTATGAGCAGCTTGAGGGTTCTTAAGAGACAGCTCTCG
TCCATCTTTATTTCCCCAGAACAGCAGAGCATTGCACATAAGAATAACAATAATAGATTGAGCTCTGTGT
CCAGTACTATTCTAAGGAATATAAATATATATTCATATATATTTATGAATATATGTATATTTATCTAATC
CCACAGCAACCTAGTGAAGTAGGTGCTGTTATTACTCCCATTTTACAGATAAGAAAACTGAGCCGCAGTA
GCTATGAAGTCATAGAACTAGAGTGGAATGTAGGCAGTTGGACCCTGAACCCTCCATCAAGGAGTAGGTA
TCCCTGAAATAAGTGAGGGGAAAATGCAGCCTTTCCAGACCCATTGGCTCTGATATCTGGAGCGGGGATC
CTGCCTTCTGAAAGGCAGAATTATTGGTGATTCCATGTGCTCTTGTTTACTTTGCTTACCAAGACCTATC
AAGATTGAGTCATTTATGCTCTCATCTCTGAGAGACTCAGTGCTCTTCATGCAGTTGGACCCAACGCTTC
ATGCTCCACTACTTACAGGGTATGGCTCTCTCGGCATGATGACCAGCGTGCTGGTTTGTCCAGATGGCAA
GACAGTAGAAGCAGAGGCTGCCCACGGGACTGTAACCCGTCACTACCGCATGTACCAGAAAGGACAGGAG
ACGTCCACCAATCCCATTGGTAAGATGGTGTTTACATTGATTTCTCACCAGAGAGTGCTCTCCAAAGCAG
CCCAGATGTGTTCCCTTGGCTGCTCAGTGTTTTGTGTGCAAAGTCATCACCAAGTAGCTGATTTGTTCTA
CTAGTAAGATCTATTTGAATATGCTTCTTAAAATATACAATATTTGCTTTTCTGTTTTGTGGGAGAACAT
AGGTTACAGAGAGGAATGTTATCAACAAGCATTTTCCTTTACCTCTGGTGCAGTGAAATGTCACATAAGG
CTACAAATCTAAAAGTCACATGGATAGATCCTTTTAACAAGTTTCAAAGATAGCACTTCAAAAAGAGATT
ACAGCCTGGAAAAAGGAGACCAATATAGACAAGAGATAGAAGAGATTGTGACCTTTTTCAAATTAGAAAA
TGCCTTAAGTTTCAAACACTAGTACCATGCCATGAAAATGTGTTTTTTTAAAGTTTCTGTTTGGTAAAAT
ATTTCTAACTCTCCTAAAACTTCTAGCCCATGTGGTTAGTATCTAAAGAAGTCTGTCACCTAAACAAAAA
ACTATTCTGTTTTGGGGAACTATGAGACATTTGGAAACCTCTATAACTAGGATGATTGGAGTGTTGTGTT
TTTTTTCCCCTCTTTTAGCTTCCATTTTTGCCTGGACCAGAGGGTTAGCCCACAGAGCAAAGCTTGATAA
CAATAAAGAGCTTGCCTTCTTTGCAAATGCTTTGGAAGAAGTCTCTATTGAGACAATTGAGGCTGGCTTC
ATGACCAAGGACTTGGCTGCTTGCATTAAAGGTTTACCCAAGTAAGTATAAGATGCCCTAGTTTCTATGG
TCAAATTAACACTTATCCTCACTGGGCTCAGAGCATCGGTGCTTTCTGGAATTATATAAATTGTTATTCA
TCATCTAGTTCAGGAGTCAGCAGATATCTGTAAATTGTCAGATAGTGAACATTTTAGGCTTTGTGGAACA
TATAGTCTTTTTGCAACTACACAACTATTCAACTTTGCCTCTGCCATTGTAGCAGGAAAGCAGTCATAGA
CAATACAGAATCAAATGGTGTGGCTGGTACCAATCAAACTTTTATGTTCAAAACCAGCCTCGATTTGGTT
CATGTGCTGGGCTGTAATTTGCTGACATCCCATCTAGTTGATAATCATTGAACGAAAAGGTTCTATTTAA
TTTTAAGAAGCAGATTATAGACAAATAGAAACAAAGATACTTCAGAGGCTATTTTATTCTACTCCTTTAT
TTTCTTTAACTTACTGATGAGAAAAGTGAGCAGCACAAGTTTAAGGTGTGCAATTGATTACAGTCCCTTA
AGTATCTGTAACCAATATTTGGGCAAATATTTGACAGCCCATATGTTTACTGAGAATTTTTCTGAGAACA
TAGTAGCCAGTATCCTGGGGGGATTGTCTCCATATCTCAACCAGTGCATCTGCAGAGGTCCCAGGTAGGG
TCAGTATACCCCATAATGGGGATATACTTGGTCACTGCATGTTTCTTAATTTAAATTTCCAACAAGATTG
CAGTGTTCGCTGGGTGCTGTGGCTCATGCCTGTAATCCTAGCACTTTGGGAGGCCGAGGTGGGTGGATCA
CGAGGTCAGGAGATCAAGACCATCCTGGCTAACACAGTGAAACCCCATCTCCACTAAAAATACAAAAAAT
TAACCAGGCGTGGTGGCGGGCACCTGTAGTCCCAGCTACTCGGGAGGCTGAGGCAGGAGAATGGCGTGAA
CCTGGGAGGCGGAGCTTGCAGTGAGCCGAGATGGTGCCACTGCACTCCAGCCTGCGTGACAGAGCGAGAC
TCCATCTCAAAAAAAAAAAAAAGATTGCAGTGTTCAAAATGGTCTTATCAGTGGAATACCTTTGTAGCCT
ATTTAACAGGCACCTGTCAGCAGGATTATGTTATCCTGTCTTTTGTTTTTCTTTTTTTTTCTTTTTATTG
AGACAGAGTCTCGCTTTGCCCAGGCTGGAGTGCAGTGGCGTGATCTTCGCTCACTGCAACCTCCGCCTCC
TGGGTTCAAGTGATTCTTCTGCCTCAGCCTCCCAAGTAGCTGGGACTTGTGCCACAACACCCGGCTAATT
TTTTTTTTTTTTTTTTTGTATTTTTAGTAGAGACGGGGTTTCACTTTGTTAGCCAGGATGGTCTCCATCT
CCTGACCTCATGATCCACCCGCCTTGGCCCCCCAAAGTGCTGGGATTATAGGCGTGAGCCACCAGGCCCG
GCCTGTCTTTTGTTTTTCTTAAGACACTGATAGAAATAACAACATTATAACTTTTTTTAAGGACATGTAA
CGGTTTGTGGGAAGGTGGGTAAAGGTTTGGCTATGCCCTGAATAAGTTTTGTTATGTTTAAGAATGAGGA
AGAAGTGTGATGATTTTGGTCTCAGCAGCATTAGTCTGAACCGAAACTCAATGATAATTTCACCCCCAAA
GCTACATAGCAAGAAACCTAACTGAGTATGTATGGCTGTCTCTACTGCCGGTTGCCTAGAGAGCCTGAGT
ACTGAGCCTGGGTCAGCCCCAGGAACCCTGAACATAGTCATTGTCTATAGATTTTGTAGCTTACTAACAT
GAATGCGTTTTCTTCCAGAAACAAATAGAATACTATTTTAAGTAGCATTTCTAGGACTTTACCACTACCT
GCTACCATATCAGAGACCAACTAAATTTTCTTTTTTTTTTCCTTCCCGTTAGTGTGCAACGTTCTGACTA
CTTGAATACATTTGAGTTCATGGATAAACTTGGAGAAAACTTGAAGATCAAACTAGCTCAGGCCAAACTT
TAAGTTCATACCTGAGCTAAGAAGGATAATTGTCTTTTGGTAACTAGGTCTACAGGTTTACATTTTTCTG
TGTTACACTCAAGGATAAAGGCAAAATCAATTTTGTAATTTGTTTAGAAGCCAGAGTTTATCTTTTCTAT
AAGTTTACAGCCTTTTTCTTATATATACAGTTATTGCCACCTTTGTGAACATGGCAAGGGACTTTTTTAC
AATTTTTATTTTATTTTCTAGTACCAGCCTAGGAATTCGGTTAGTACTCATTTGTATTCACTGTCACTTT
TTCTCATGTTCTAATTATAAATGACCAAAATCAAGATTGCTCAAAAGGGTAAATGATAGCCACAGTATTG
CTCCCTAAAATATGCATAAAGTAGAAATTCACTGCCTTCCCCTCCTGTCCATGACCTTGGGCACAGGGAA
GTTCTGGTGTCATAGATATCCCGTTTTGTGAGGTAGAGCTGTGCATTAAACTTGCACATGACTGGAACGA
AGTAGGAGTGCAACTCAAATGTGTTGAAGATACTGCAGTCATTTTTGTAAAGACCTTGCTGAATGTTTCC
AATAGACTAAATACTGTTTAGGCCGCAGGAGAGTTTGGAATCCGGAATAAATACTACCTGGAGGTTTGTC
CTCTCCATTTTTCTCTTTCTCCTCCTGGCCTGGCCTGAATATTATACTACTCTAAATAGCATATTTCATC
CAAGTGCAATAATGTAAGCTGAATCTTTTTTGGACTTCTGCTGGCCTGTTTTATTTCTTTTATATAAATG
TGATTTCTCAGAAATTGATATTAAACACTATCTTATCTTCTCCTGAACTGTTGATTTTAATTAAAATTAA
GTGCTAATTACCATT
SEQ ID NO: 36 (NM_001282386.1, IDH1 isoform 2 mRNA):
AGGGGGCGTGGAGGGGCGGAGCTCCAGCGGCCTTCAGAGGGGTCGCTAGGCCACACGGGGCGAGAGGGGG
CTGGGGGAAACACCCCCGCCAGTTCCGGGTGCCTGGAGTTTAAAAGGCGATAAACTACATTCAGTTGAGT
CTGCAAGACTGGGAGGAACTGGGGTGATAAGAAATCTATTCACTGTCAAGGTTTATTGAAGTCAAAATGT
CCAAAAAAATCAGTGGCGGTTCTGTGGTAGAGATGCAAGGAGATGAAATGACACGAATCATTTGGGAATT
GATTAAAGAGAAACTCATTTTTCCCTACGTGGAATTGGATCTACATAGCTATGATTTAGGCATAGAGAAT
CGTGATGCCACCAACGACCAAGTCACCAAGGATGCTGCAGAAGCTATAAAGAAGCATAATGTTGGCGTCA
AATGTGCCACTATCACTCCTGATGAGAAGAGGGTTGAGGAGTTCAAGTTGAAACAAATGTGGAAATCACC
AAATGGCACCATACGAAATATTCTGGGTGGCACGGTCTTCAGAGAAGCCATTATCTGCAAAAATATCCCC
CGGCTTGTGAGTGGATGGGTAAAACCTATCATCATAGGTCGTCATGCTTATGGGGATCAATACAGAGCAA
CTGATTTTGTTGTTCCTGGGCCTGGAAAAGTAGAGATAACCTACACACCAAGTGACGGAACCCAAAAGGT
GACATACCTGGTACATAACTTTGAAGAAGGTGGTGGTGTTGCCATGGGGATGTATAATCAAGATAAGTCA
ATTGAAGATTTTGCACACAGTTCCTTCCAAATGGCTCTGTCTAAGGGTTGGCCTTTGTATCTGAGCACCA
AAAACACTATTCTGAAGAAATATGATGGGCGTTTTAAAGACATCTTTCAGGAGATATATGACAAGCAGTA
CAAGTCCCAGTTTGAAGCTCAAAAGATCTGGTATGAGCATAGGCTCATCGACGACATGGTGGCCCAAGCT
ATGAAATCAGAGGGAGGCTTCATCTGGGCCTGTAAAAACTATGATGGTGACGTGCAGTCGGACTCTGTGG
CCCAAGGGTATGGCTCTCTCGGCATGATGACCAGCGTGCTGGTTTGTCCAGATGGCAAGACAGTAGAAGC
AGAGGCTGCCCACGGGACTGTAACCCGTCACTACCGCATGTACCAGAAAGGACAGGAGACGTCCACCAAT
CCCATTGCTTCCATTTTTGCCTGGACCAGAGGGTTAGCCCACAGAGCAAAGCTTGATAACAATAAAGAGC
TTGCCTTCTTTGCAAATGCTTTGGAAGAAGTCTCTATTGAGACAATTGAGGCTGGCTTCATGACCAAGGA
CTTGGCTGCTTGCATTAAAGGTTTACCCAATGTGCAACGTTCTGACTACTTGAATACATTTGAGTTCATG
GATAAACTTGGAGAAAACTTGAAGATCAAACTAGCTCAGGCCAAACTTTAAGTTCATACCTGAGCTAAGA
AGGATAATTGTCTTTTGGTAACTAGGTCTACAGGTTTACATTTTTCTGTGTTACACTCAAGGATAAAGGC
AAAATCAATTTTGTAATTTGTTTAGAAGCCAGAGTTTATCTTTTCTATAAGTTTACAGCCTTTTTCTTAT
ATATACAGTTATTGCCACCTTTGTGAACATGGCAAGGGACTTTTTTACAATTTTTATTTTATTTTCTAGT
ACCAGCCTAGGAATTCGGTTAGTACTCATTTGTATTCACTGTCACTTTTTCTCATGTTCTAATTATAAAT
GACCAAAATCAAGATTGCTCAAAAGGGTAAATGATAGCCACAGTATTGCTCCCTAAAATATGCATAAAGT
AGAAATTCACTGCCTTCCCCTCCTGTCCATGACCTTGGGCACAGGGAAGTTCTGGTGTCATAGATATCCC
GTTTTGTGAGGTAGAGCTGTGCATTAAACTTGCACATGACTGGAACGAAGTATGAGTGCAACTCAAATGT
GTTGAAGATACTGCAGTCATTTTTGTAAAGACCTTGCTGAATGTTTCCAATAGACTAAATACTGTTTAGG
CCGCAGGAGAGTTTGGAATCCGGAATAAATACTACCTGGAGGTTTGTCCTCTCCATTTTTCTCTTTCTCC
TCCTGGCCTGGCCTGAATATTATACTACTCTAAATAGCATATTTCATCCAAGTGCAATAATGTAAGCTGA
ATCTTTTTTGGACTTCTGCTGGCCTGTTTTATTTCTTTTATATAAATGTGATTTCTCAGAAATTGATATT
AAACACTATCTTATCTTCTCCTGAACTGTTGATTTTAATTAAAATTAAGTGCTAATTACCATTAAAAAAA
AAA
SEQ ID NO: 37 (NM_001282387.1, IDH1 isoform 2 mRNA):
CGTGGAGACCTGGAAGGAGGTACCGATCCAGCCTTGATTGCAGCCCTCTTGCACAACGGCCATAAGGACG
GGTGCAGGCCAAGAGCAAGGACATGCAAACCCCAAGACCTGCTAACGGGCGGCGAAGCGGGGGCACGCCC
TCGCACACGCAGAGATAAATTGTGCTCCCATGACCTTTATTTGGAAAGTGCCTGCGGGCCTAAAATTGGC
CTTTGTCCCACCGAGTACACTCAGCACTGTACTTTAAACCGGATAAACTGGGCTGTCTGGCAGGCGATAA
ACTACATTCAGTTGAGTCTGCAAGACTGGGAGGAACTGGGGTGATAAGAAATCTATTCACTGTCAAGGTT
TATTGAAGTCAAAATGTCCAAAAAAATCAGTGGCGGTTCTGTGGTAGAGATGCAAGGAGATGAAATGACA
CGAATCATTTGGGAATTGATTAAAGAGAAACTCATTTTTCCCTACGTGGAATTGGATCTACATAGCTATG
ATTTAGGCATAGAGAATCGTGATGCCACCAACGACCAAGTCACCAAGGATGCTGCAGAAGCTATAAAGAA
GCATAATGTTGGCGTCAAATGTGCCACTATCACTCCTGATGAGAAGAGGGTTGAGGAGTTCAAGTTGAAA
CAAATGTGGAAATCACCAAATGGCACCATACGAAATATTCTGGGTGGCACGGTCTTCAGAGAAGCCATTA
TCTGCAAAAATATCCCCCGGCTTGTGAGTGGATGGGTAAAACCTATCATCATAGGTCGTCATGCTTATGG
GGATCAATACAGAGCAACTGATTTTGTTGTTCCTGGGCCTGGAAAAGTAGAGATAACCTACACACCAAGT
GACGGAACCCAAAAGGTGACATACCTGGTACATAACTTTGAAGAAGGTGGTGGTGTTGCCATGGGGATGT
ATAATCAAGATAAGTCAATTGAAGATTTTGCACACAGTTCCTTCCAAATGGCTCTGTCTAAGGGTTGGCC
TTTGTATCTGAGCACCAAAAACACTATTCTGAAGAAATATGATGGGCGTTTTAAAGACATCTTTCAGGAG
ATATATGACAAGCAGTACAAGTCCCAGTTTGAAGCTCAAAAGATCTGGTATGAGCATAGGCTCATCGACG
ACATGGTGGCCCAAGCTATGAAATCAGAGGGAGGCTTCATCTGGGCCTGTAAAAACTATGATGGTGACGT
GCAGTCGGACTCTGTGGCCCAAGGGTATGGCTCTCTCGGCATGATGACCAGCGTGCTGGTTTGTCCAGAT
GGCAAGACAGTAGAAGCAGAGGCTGCCCACGGGACTGTAACCCGTCACTACCGCATGTACCAGAAAGGAC
AGGAGACGTCCACCAATCCCATTGCTTCCATTTTTGCCTGGACCAGAGGGTTAGCCCACAGAGCAAAGCT
TGATAACAATAAAGAGCTTGCCTTCTTTGCAAATGCTTTGGAAGAAGTCTCTATTGAGACAATTGAGGCT
GGCTTCATGACCAAGGACTTGGCTGCTTGCATTAAAGGTTTACCCAATGTGCAACGTTCTGACTACTTGA
ATACATTTGAGTTCATGGATAAACTTGGAGAAAACTTGAAGATCAAACTAGCTCAGGCCAAACTTTAAGT
TCATACCTGAGCTAAGAAGGATAATTGTCTTTTGGTAACTAGGTCTACAGGTTTACATTTTTCTGTGTTA
CACTCAAGGATAAAGGCAAAATCAATTTTGTAATTTGTTTAGAAGCCAGAGTTTATCTTTTCTATAAGTT
TACAGCCTTTTTCTTATATATACAGTTATTGCCACCTTTGTGAACATGGCAAGGGACTTTTTTACAATTT
TTATTTTATTTTCTAGTACCAGCCTAGGAATTCGGTTAGTACTCATTTGTATTCACTGTCACTTTTTCTC
ATGTTCTAATTATAAATGACCAAAATCAAGATTGCTCAAAAGGGTAAATGATAGCCACAGTATTGCTCCC
TAAAATATGCATAAAGTAGAAATTCACTGCCTTCCCCTCCTGTCCATGACCTTGGGCACAGGGAAGTTCT
GGTGTCATAGATATCCCGTTTTGTGAGGTAGAGCTGTGCATTAAACTTGCACATGACTGGAACGAAGTAT
GAGTGCAACTCAAATGTGTTGAAGATACTGCAGTCATTTTTGTAAAGACCTTGCTGAATGTTTCCAATAG
ACTAAATACTGTTTAGGCCGCAGGAGAGTTTGGAATCCGGAATAAATACTACCTGGAGGTTTGTCCTCTC
CATTTTTCTCTTTCTCCTCCTGGCCTGGCCTGAATATTATACTACTCTAAATAGCATATTTCATCCAAGT
GCAATAATGTAAGCTGAATCTTTTTTGGACTTCTGCTGGCCTGTTTTATTTCTTTTATATAAATGTGATT
TCTCAGAAATTGATATTAAACACTATCTTATCTTCTCCTGAACTGTTGATTTTAATTAAAATTAAGTGCT
AATTACCATTAAAAAAAAAA
SEQ ID NO: 38 (NM_005896.4, IDH1 isoform 1 mRNA):
GGGTTTCTGCAGAGTCTACTTCAGAAGCGGAGGCACTGGGAGTCCGGTTTGGGATTGCCAGGCTGTGGTT
GTGAGTCTGAGCTTGTGAGCGGCTGTGGCGCCCCAACTCTTCGCCAGCATATCATCCCGGCAGGCGATAA
ACTACATTCAGTTGAGTCTGCAAGACTGGGAGGAACTGGGGTGATAAGAAATCTATTCACTGTCAAGGTT
TATTGAAGTCAAAATGTCCAAAAAAATCAGTGGCGGTTCTGTGGTAGAGATGCAAGGAGATGAAATGACA
CGAATCATTTGGGAATTGATTAAAGAGAAACTCATTTTTCCCTACGTGGAATTGGATCTACATAGCTATG
ATTTAGGCATAGAGAATCGTGATGCCACCAACGACCAAGTCACCAAGGATGCTGCAGAAGCTATAAAGAA
GCATAATGTTGGCGTCAAATGTGCCACTATCACTCCTGATGAGAAGAGGGTTGAGGAGTTCAAGTTGAAA
CAAATGTGGAAATCACCAAATGGCACCATACGAAATATTCTGGGTGGCACGGTCTTCAGAGAAGCCATTA
TCTGCAAAAATATCCCCCGGCTTGTGAGTGGATGGGTAAAACCTATCATCATAGGTCGTCATGCTTATGG
GGATCAATACAGAGCAACTGATTTTGTTGTTCCTGGGCCTGGAAAAGTAGAGATAACCTACACACCAAGT
GACGGAACCCAAAAGGTGACATACCTGGTACATAACTTTGAAGAAGGTGGTGGTGTTGCCATGGGGATGT
ATAATCAAGATAAGTCAATTGAAGATTTTGCACACAGTTCCTTCCAAATGGCTCTGTCTAAGGGTTGGCC
TTTGTATCTGAGCACCAAAAACACTATTCTGAAGAAATATGATGGGCGTTTTAAAGACATCTTTCAGGAG
ATATATGACAAGCAGTACAAGTCCCAGTTTGAAGCTCAAAAGATCTGGTATGAGCATAGGCTCATCGACG
ACATGGTGGCCCAAGCTATGAAATCAGAGGGAGGCTTCATCTGGGCCTGTAAAAACTATGATGGTGACGT
GCAGTCGGACTCTGTGGCCCAAGGGTATGGCTCTCTCGGCATGATGACCAGCGTGCTGGTTTGTCCAGAT
GGCAAGACAGTAGAAGCAGAGGCTGCCCACGGGACTGTAACCCGTCACTACCGCATGTACCAGAAAGGAC
AGGAGACGTCCACCAATCCCATTGCTTCCATTTTTGCCTGGACCAGAGGGTTAGCCCACAGAGCAAAGCT
TGATAACAATAAAGAGCTTGCCTTCTTTGCAAATGCTTTGGAAGAAGTCTCTATTGAGACAATTGAGGCT
GGCTTCATGACCAAGGACTTGGCTGCTTGCATTAAAGGTTTACCCAATGTGCAACGTTCTGACTACTTGA
ATACATTTGAGTTCATGGATAAACTTGGAGAAAACTTGAAGATCAAACTAGCTCAGGCCAAACTTTAAGT
TCATACCTGAGCTAAGAAGGATAATTGTCTTTTGGTAACTAGGTCTACAGGTTTACATTTTTCTGTGTTA
CACTCAAGGATAAAGGCAAAATCAATTTTGTAATTTGTTTAGAAGCCAGAGTTTATCTTTTCTATAAGTT
TACAGCCTTTTTCTTATATATACAGTTATTGCCACCTTTGTGAACATGGCAAGGGACTTTTTTACAATTT
TTATTTTATTTTCTAGTACCAGCCTAGGAATTCGGTTAGTACTCATTTGTATTCACTGTCACTTTTTCTC
ATGTTCTAATTATAAATGACCAAAATCAAGATTGCTCAAAAGGGTAAATGATAGCCACAGTATTGCTCCC
TAAAATATGCATAAAGTAGAAATTCACTGCCTTCCCCTCCTGTCCATGACCTTGGGCACAGGGAAGTTCT
GGTGTCATAGATATCCCGTTTTGTGAGGTAGAGCTGTGCATTAAACTTGCACATGACTGGAACGAAGTAG
GAGTGCAACTCAAATGTGTTGAAGATACTGCAGTCATTTTTGTAAAGACCTTGCTGAATGTTTCCAATAG
ACTAAATACTGTTTAGGCCGCAGGAGAGTTTGGAATCCGGAATAAATACTACCTGGAGGTTTGTCCTCTC
CATTTTTCTCTTTCTCCTCCTGGCCTGGCCTGAATATTATACTACTCTAAATAGCATATTTCATCCAAGT
GCAATAATGTAAGCTGAATCTTTTTTGGACTTCTGCTGGCCTGTTTTATTTCTTTTATATAAATGTGATT
TCTCAGAAATTGATATTAAACACTATCTTATCTTCTCCTGAACTGTTGATTTTAATTAAAATTAAGTGCT
AATTACCA
SEQ ID NO: 39 (NC_000015.10: c90102468-90083045 Homo sapiens chromosome 15,
GRCh38.p13 Primary Assembly, IDH2 genomic sequence):
AGCCCGCGGCCAGGCAGCCGGGAGGAGCGGCGCGCGCTCGGACCTCTCCCGCCCTGCTCGTTCGCTCTCC
AGCTTGGGATGGCCGGCTACCTGCGGGTCGTGCGCTCGCTCTGCAGAGCCTCAGGCTCGCGGCCGGCCTG
GGCGCCGGCGGCCCTGACAGCCCCCACCTCGCAAGAGCAGCCGCGGCGCCACTGTGAGTGCCGCGCGGAG
GGTCCAGGCAGGCGCGCGCCCCCAGCTGCGACGTGGCGGCTTCCCAGGCTGGGGCCGGGAAGCACGTCCC
GCCAGCCCGCAGCCCGGGGACGGCGGGGTCAGGCGCACAAAGGGCCGCGGGGCAGCTTCGCTCCGCGCTC
CCCCGCCCGGCGCTTCCCGGCCGTGCCAGGCGCTGGGGTGGGGACTTGAGGGACGGTGGCAACTGGTGGC
CCGGGCGACGGCCAGCTCTGCGCGGGCGGGGGCTCGGGGCTGCCGCGCGCCCCTAGGTATTTCGGGAGGG
GGTCCTGAGAGTGTCCCGGACCCGAAGGGTGGCCTGCCCGCCCTGCGCCCCTGGAACGAGACGAGCACCC
GTGGGGGGCTGGAGCACCCCGCGCGCTCCCCTGGCGAGAGGGAGGGTCGTGGCTCGGCCCCTGCTCAGAC
AAAGGCTGGGAGGCGGGAGACATGCACTTCCCCTTCCTTTTCAGCCAGGCGCGCGCTGATACCAGGCCCA
CGTCAGCTATTTTTGGAGCCTTTTACACGACAGCTGGAGGAGCGTCCTTTTTAATTTTCCCCTTTTGTTT
GGCCGCCCCCACCCCCACCCCTTCGCCTTCATCGCTGCACTTGAGGCTCCATCCTGGGGCCTCTCCTTGA
CTTGACCTGCCTTGGCAGGCACATGCCCTCCCTGCCTGGCTCACTCGCCGCAGAGACCTGGCAGCCCGCG
CAAAATGTCACTTTGCGGAATCGTTCCCACGGCTTCTGGGTACCCTTAGTTCCCTGCTTAGGAGGGAAGA
CAGTAGTCGGGTCGTAATAAGCAAGACTTAGCCCGAGCCTCCGTTGCCAACGCAGGCTGCCTTGCTTGGC
GTGTGGGCATCGGCCTGCCCCCTCACCCTGGCTACCCAACACAGCTACAAAAGGCAGGGAACAATGTAGG
TCCCTTGGCCCTGCCTAATGCCTGTTGCCATGGAAACCCCTATCCTAATCTGGCCAGGAGCCCCTTGCAG
TGAGCCAGGAGAGTGAGGAAGAGGGGATGGGGCCCGCTGGCCCTGACCTGGCCAGAGGAGGTAATGGTTA
ACCGGATTGTGGGAGCAGCTGACTAGAGCCGGGGGGGTAGGGAGGCTTGGGCCCCAGTCCTACCTTCCCT
GCCAAGGAGAAAGGGGCATGTCTGCTTTTGTACCTCTGGGAATCTACCTCAGGGATCTGCCCAACAACTC
CCAGGTTCCAAGTGCCCCAGGACCCAAAGATTCCCATATAGCACACCACCTTCAGCAAAAATATGGTGGA
AGTTAGCCACATTCTTTAATTCTCCCTTTTTTTCTGCCTTGATGGTAAAAAAAAAAAAAAGAAACAAACA
ACCCGAAAAACACACACAAACAGGAACACAAAGCGCCTTTTAAGGGTCCTGAATTTGGATACAGAAAAAG
CAGTCAGTTGCAGGCCCTTCTAGAAGTCCCCATGTGGGTTGGTTATGAATGGGGAAATTGCTTGGTGTTG
GAGTTGGACATCAGACTCTACAGTTGCGACTCTGCCAGCTTGTTACCTGGCAACGCAGCTGGAGACAGAA
TACTTGGCATTCCCCTTGATGACAAGGAAAGAATATTTTGGGGCTAAAAGAGCCCCGTGTCTTTGCTGCC
TGGTAATTCCTGCACGCATCTTTTCCTATTTTGCAACGCCATAGGCTTCCAGCGACTGCTGGTGATGTTT
CTGGTAAGTTAGAGCTTGGGGCAGTGCGGACCAGTTCTGTGAACGGCTCAGTGGCAGAATTACTTATCTA
GCCTTCCTTCACTCCTAACCCAGAAACTTCTTAATTCTTGCCCCTTAAGTGTGCAGATCTTTCTGCCACG
TCAGATAAGGCCCTAGCTGCCCTGCCAGAGCTGATTGGGGTGGTTGTGTTTAGGCCTAGTAGGTACTGAG
GAAAAGCCTCTTTGGAATGGCTGAGGCCTGCTCCCACATCTTCTGTGTGAAGTTGAGGGCCCTTGGGCTG
GTTCCATTCAGGGTATGAGGTGCTGGAAACTGCTCTCAGATTACTGGAAGCCAGGGTCTGTGGCCTTCCC
TGGGGTTTATACTCTGTAAACCCATGTTAAAAATACCAGCAGATTGCAAATATTAGTACACTGTAGGTTG
TAAGCTTCTTCAGGGCATGGGGCCTATTGCTGGAGAGAGATGCCACCCGTGTACATCTGGTGCAGGCTGC
TGGTAAGAACTCACTGGCCCCTGTGTGGCAATGCATACCAGATGGGTGAGAGGGGAGGAAACACATTCAG
TGAAACAGAAGGTGAGGGCAGGGCAGCCCTGACCACAGATGGAGTTGACTTCAACTTGACCTTTATTTAT
GTCTTTGACAAAAGTAGTATACTCCTACTTTGTGCCAGACACCATGCCGGGTGCTGGGGATAGAGCTGGG
AATAAAACCAGCAGAGACTCTTTCAAAACCCTTAGGAGCTCAAATTCTGATGTGGGAGGGGGCAGACAGT
AAGCAAGATAGATGTTGACTAGTATGATATATGGGTACAAGAGCTAAGGAAGGCCAGGCACTGTGGCTTA
CACCTGTAATCCCAGAATTTTGGGGGGCTGAGGTGGGAGGATTGCTTGAGGCTGGGAGCTCCAGACCAGC
CTGGGCAATATAGTGAGACCCCATCTCTACAAAAATAAAAAATAAAATTAGCCAGGCACGATGGTGCATG
TCTGTGGTCTTAGCTACTCAGGAGGATCATTTGAGCCCAGGAGGTCAAGGCTGCAGTGAGGTACAATTGT
GCCACTGCACTCTAGCCTGGGTGACAAAGCAAGACCCTGCCTCCCCCTTCTCCCCCCAAAAAGAGAAGAG
CTAAGGAGAAACAGGCAAGAAAGGTGTGTGCACAAAAGTTTGTATAGGGCAGCCAGAAAAGTCCTCACTG
AAGGAATCGCTGAGGAGGGGAGGAAGCAGGCCATTTTCGGTATTGGGGGAAGAGGGACTCGCAGGTGCCA
AGGGCATGATGGAAGAGCAGAGAGGATGGTGCGAAGGCTGGCTCAGCAGGCACCAGGTGGAGTGACAGGA
TGAGGTTCGGCAGTGGGGTCTGTAGAGGGCTCACAGGCCATTTTTGAGAGCTTTCGCTTTTACTTTGCAG
GGTTTTGAGAGGAGTATCGTTACCTGATTTCCATCTTATCGGGAGCATTCTAGCTTCAGGGACCTGGCAA
GAGAGGATGATGGCTTGGACCTGGGTGGTGGCGCTGGGGATGGTGAAGAAGTGGTTGGATTCTGGATATA
TTTTGAAGGAGGAGCCAGTAGTGGGGAAAGGTTCCAATTCAGGCGATTTTACATAAAAATCTAGATACGC
AGCAGGCACTCACTTATGGCAAGGGTGGGCTTGTCTGAGGGTGGCTTCTGGTTTTGGTGGTTTGGATGTG
AGAGAAGGGAAGGAATGAAGGATGACACCAAGGAGTCTACTAGAGTTTGTTCCAAAATGGCTGTTCTGGC
CGCGTGCGGTGGCTCCCAGGACTTTGGGAGGCCGAAGCAGGCGGATCAGTTGGGGCCAGGAGTTCGAGAC
CTGCCTGGCCACCATGGTGAAACCCGGTCTCTACTAAAAATACAAAAATTAGCTGGGTGTGGTGGCAGGC
GCCTGTAGTCCCAGCTACTCAGGGGCTGAGGCAGGAGAATTGCCTGAACCCAGGAGGTGGAGCTTGCAGT
GAGCCGAGATCACGCCACTGCCCTCCAGTGTAGGCGACAGAGTGAGACTCTGTCTCAATACATACATACA
TACATACATACATACATGCATGCATACATACATACATACATACATAAAATATACAAAGTTGCTGTCCTGC
CTTCTTGAGCCCCCAGGAATTGGAACCTTTCCCCACTCTCTGATCTTTTGCTGGGACATGCTGACTGAGC
AGCCTTTCTGCTGAGAGCATGGGTAGGAATGTGGAGGGAGCAGTTATGCCTGGGGCTGGCTGCCTGCGGC
TGGACCTCTAGCCTCCTGCTGGGAGCAGCAAGAAGCCAGGGATGTGTTTTGAAATCCTTACTTGGTATTG
GGTGCTAGGAGCCCCAGACAGGGAGGAGAGTCAGCCTAACGTGCCTCTGGTGACTGGTGTAATTCCATCC
TGCACTCTCGCCAAGAGGACAGTGTTTGTGCAATGCATCAGCTGAAGATTCGTGGTGGCTGTCACTTCGG
AGGTTTTCAGGGAGGCCTAGCTGCTGCTCTGTGTATTTCAGGTAGCTTGTCACCACTTATCTCTCAGAGA
AGGGCAGACCATGGCTGTATCTCCACCATACAGGCTGAACAGCAGACAGGGGTCTCAAACTCCAGTGCCC
TGGGCCACACTGGAGGGCACTTGGCTGTCATGGAGTGAAGGGAGAGGCCGCCTTCAGCCCAGCCCATCCT
TGCCATGAGTGAGTGCCTGCTGCATTTCTAGATTTTTATATAAAATCTCCTGATTTTTTTTGACTGGTTT
AAATTTTTATTATTTTGGTAAAAAACGTATAAAACTTACCATCTTAGTCATTGTTCAGTGTACAGTATTG
TTAAGTACATTCGCACTGTTGTGCAGACATTACCACCATCCATCACCAGAACTTTGTCACCTTGCAAAAC
CGAAACTCTCTACCCATGAAACAATACCTCCCCACTTCCCGTCCCCAGTCCCTGGCAGCCACCTTTTTAC
TTTCTGTTTCTATGAATTTGACTATTCTAGGTACCTCATGTAAGTAGAAGCATATAGTATTTGTCTTTTG
TGACTGGTTTATTTCACTTAGTTAGCATAATGCCCCCCAGGTTCATCCACTTTGTAGCAGGTGTCAGAAT
TTCCCTTCTTTTCAAGGCTGAATCATATTCCATTGTATGTAGATAACAGTAGTCCCCCTTATCCTTGGAG
GATGCATTTCAAGATCCCCAGTGGATACCTGAAACTGCAAAGCCTGTAGATACTATGTTTTTTTCCTATA
CATACAGATCTATGATAAAGTTTAATTTATGAATTAGGAACAGTAAGAGATTAGCAATAATAACTAGTAA
TAAAATAGAACAATTATAACAATATACTGTAATGAAAGTTACATGAATGTGATCTCTTAAAATATCTTAC
TGTACTGTACTCACCTATTTTCAGACTATGGTTGACTGTGGGTAACTGACACCACAGGTAACTGACGTCA
TGGAAATAAAACCTTTGATAAGGGGGGACTGTATCACATTGTCTTTATCCATTCATCTGTGATGGACACG
TGGGTTGCTTCTATCTTTTGCTGTTGTGAACATTGCTGCTATGAATGTGGGCATACATATATCTCTTTCA
GACCCCGCTTTCGATATTTTTGGATGTATATACAGAAGCAGAATTGCTGGATCATATAGTAATTCTTGTC
CTTTTCTTTTCTTTTTTTTTTTCGAGACGGAGTCTTGCTCTCTTTCGCCCATGCTGGAGTGCAGTGGCGC
GATCTCAGCTCACTGCAAGCTCCGCCTCCCGGGTTCATGCCATTCTCCTGCCTCAGCCTCCCCAGTAGCT
GGGACTACAGGCGCCTGCCACCACGCCTGGCTAATTTTTTGTATTTTTAGTAGAGTCAGGGTTTCACTGT
GTTAGCCAGGATGGTCTCGATCTTCTGACCTCGTGATCCACCCGCCTCAGCCTTCCACAGTGCTGGGATT
ACAGGTGCGAGCCACCGTGCCCGGCAGTAATTCTTTTCCTTTTAGCTAAGGAACACCCATACTGTTTTCC
ACAGCAGCTGCACCATTTCACATTCCCACCAACAGGGCACAAGAGTTCCAATTTCTCCACATCCTTGCTA
GCACTTTATTTTCTGTTTTGTTTTTCTTTTCCTGATAGTAGCTCTCTTAGTGGTTGTGAGGGGATAGTGG
GGTGATATCTCATTATGGTTTTGATTTGCATTTCCCTGATGATTAGTGATGTTGAGCATCTTTTCATGTG
CTTGTTGGCCATTTGTATATCATCTTTGGACTTGAAATGTCTATTTAAGTCTTTTGTCCATTATATTTAT
TTATTTATTTTGAGATGGAGTCTTCCTGTTACCCAGGCTGGAGTGCAGTGGCATGATCTCTGCTCACTGC
AACCTCCACCTCCTGGGTTCAAGCAATTCTCCTGCCTCAGCCTCCCAAATAGCTGGGACTACAGGTGTGT
GTCACCACACCAGGCTAATTTTTTTTCTATTTTTAGTAGACACAGAGTTTCACCATTGTGGCCAGGCTGG
TCTCGAACTCCTGACCTCAAGTGATCTGCCCTCTTCAGCCTTCCAAAGTGCTGGGATTACAGGCATGAGC
CACGGTGCCCAGCCAGTCTTCTGTCCATTTTAACGTTGAGTTATTTGTTTTATTGTTGAGATTTACTGAT
TTTTAAAACTTGGTCACCAATTAAGATTTTTTTAAAAAACCAGTACCTGGAACAAGCAGAACAAGTCTCT
GCTGGCCTTGGCTCATGTTGCTGTTCTCAGTTCCCCTGGGATGTTTGCTTCCAGGGAGGCCTGGGCACTC
CTGACTTTGGTGACAGCCCTGCTTGCTCTCTCTCTGTTACCTGTAGGTTTCCCCTTCCACATTCACTGCC
TCCGGGCAGGCACTGTTGTGTTTTAGGCATTTCAGTATCCCAGTGGGGTACTGGATAAGTGCTTAGTTAG
CACCTGCCGGTGAGTAAATAATCAGGTCCTGTGGTCTCTGAGGGTTTGACACATCAGGTCTTTGATCTTG
ACCTTGAACTCTCCTAGCCATGCCTGCCTGAAGCCCAACCGTGGAGCCCAGGCTGAGGCTGAAACCTGAA
GAACTGGCCCCAAGTGAGTCTCTCCTTAGGGCTCCTGATTATGATAGTTCATTTTTTTTTTTTTTTAACA
AATTTGATTAGCTGTCCTCAAATTAACTCATAGGAGATGCTCCCCAGAACTAAACTAGCTCTGCAACAGC
TCAACAGACTTTCCGCAGGTTCTGCAGCTCTTTCCACTCAAGCTTCAGAAAAAAGTGAAGGAATATTGTA
GATTGGGTTCAAAATCCCCATCGCGTCTTGGGAAAGGCAGTAGAGCCAGCAGCCAGCCACCTTTTGTTCT
CTAGGGAATGGAATAGTCCAAGGCTGTGTTTGTGTTCTGCTTCTCTACATCTGACCCACGGGTCTTCCAG
AGGCTGCATTTGGGTCAGGGACCTGGGCTCTATCTTGCCTCTGATGCAGACCACACTCTCTGAGTCTCAT
TATTCCCCCTAAATGATCTCTAAGATTATTTTTAGATTAAAAAAAAAAAACAAACTCTTTGCATATCTTT
GGAAAGTCAGTTGTTTGAGGTCTGTTAGGCTTTAATCACTTTTGTTTTAAATGTGCCGAGGTGGTTTCTA
GGCCTGCTTTTCACAGGTATTCTGTCCCTTGTCAGTTCTTCCCACCCAGTTTTTTTTGTTTGTTTGTTTG
TTTTTTTGAGATGGAGTCTCGTTCTGTTGCCCAGGCTGGAGTGCAGTGCCGTGATCTTGGCTTACTGCAA
CCTCCACCTCCCGGGTTCAAGCGATTCTCCTGCCTCAGCCTCCTCGGTAGCTGGGATTATAGGCACGTGC
CACCATGCCTAATTTTTCTATTTTTAGTAGAGATGGGGTTTCACCATGTTGGCCAGGCTGGTCTTGAACT
CCTTACCTTGAGTGATCCACCTGCCTCAGCCTCCCAAAGTGCTAGGATTACAGGCATGAGCCATTACACC
CAGCCCCAACCCAGTGTTTTCAAATAATCATTCTCTTCCCTTCACCTGATTTGCTGTGTCTAATCTAGAG
TGGAGCTGAAGACACATCACCACCTCTCTAAGGACTGTGTGTAACACAAGAGTAATTGGGCTGTAAGTGA
GGCTGAGGAGTGCTGCTTTGAGGAAAGGGCACAGCTCTCAGCTGAGCTGGTGAGATTTCCCTGTGACTGA
AGTTTTATTAGATCTTGTTCCCTGCTCTGCATTTGCTGCTTCTTCCCAGGCTGTGGGCAGAGGAGCAGGC
TGGTGCCCCGGTGCCCCTATCTGCTGCAGGCTGAGGTTCAACAAGCCTTGTCCAAGGCAGCTGTACTGAT
TCATTGTTAGGTGGCCAGTTCCTGAGTTTTCTTTGAAATTCACTTCCCAGACTGTTGGGTATTCCTGCGA
GTCAGACTCTCCTTCAGTAGCGTCCCCTGCCCCCTACCCTCCACTGGTCTCCCTGGACCTCTGATCAGCC
CCTCATTGAGTCCTTTGATGCTTCTCTGGTAGGACTGACCCACCTGCGTCGGCTGGGATCAAGGGTTCTG
AGTTCCATTCGCAGGTGCTGTAAGCCTGTCCTTCCTGTTGAATAGGGTCAGAGTCCTCGTGACTCTGCTG
TTTTCAAGCTCTGTAACCTTGGGCAGTAAGCTGTTAACCTCTCTGAGCCTCAGTTTCCCACACTTAAAAT
GGAGATAATGGCCAGGCGTGGTGGCTCACGCCTGTAATCCCAGCACTTTGGGAGGCCAAAGTGGGTGGAT
CACCTGAGGCTAGGAGTTCGAGACCAGCCTGGCCAACATGGCAAAACCCCGTCTCTACTAAAAATACAAA
AATTAGCCGGGCCTGGTGGCGTGTGCCTGTAATCCCAGCTACTCAGGAAGCTGAGACAGCAGAATCGCTT
GAACCCGGGAGGCAGAGGTTGCAGTGAGCCGAGATCACGCCATTGCATTCCAGCCTGGGTGACAGACCAA
AGACTCTGTCTCAAAAAATAAATAAATAAATAAATAAATTAATTAATTAAATAAAATGGAGGTAATGATA
CCTCCTTGGGGTTGTTTTTGGGATTAAATGGAATCACATTGTACAGCATTGGTACACTGTAAGGCACATA
GTAGGCACTCAATACCTATCTATACTATTTCCTTTATCACCACAAGTTAGTTGAGGGCTCTTGGGTTGCA
CGCAACAGACAAAGTTAGTGACTGGTTAACTTGGAGAGGATTTTATTATGAGCTACACAGCGTAGCTCAT
AGAATTGCAGGGAAAACTATAAAGAACTAAGTCTTGGACAAAAGTAATATCTTGGCATGGGCCTGTAGCC
CCAGCTACTAAGGAGGCTGAGGTGCAAGGATTGCTTGAGCCCCGGAGTCTGAGCCTGGCTTGGGCAACAG
AGAGAGATAAAGAGACCCTGACTCTAAACAATAAGTAAATACATAAAAGAGGACCCAGGCCCTCCTTAGG
AACTGGATGGCAGGAGCCCCTGGACAACCTCTTACTGATTTGGTGCTGGATGAGGCAGCCGCAGCTAGCC
AGTGTTCTTTGTGTTGATCTCCTCTGCCTTCATATTCCAGGGGGAGAGGGTCTGACAGGCCAGACTGGCC
CCCACCCTCTTGGCCACTGGTGGGCTGGGCATGTTTTTGGGTAGTTCTACCTAGCCAGATTAAAGTGGGG
GTGGGGTGGGGAGTCCAGCCAGCCTGGGCAACAGGGCAAGACTCCATCTCTACAAAAAAATTTTAAAATT
AGGCTGGCACGGTGGCTCACGCCTGTAATCCCAGCACTTTGCGGGGGTCAAGGCAGGTGGATCACTTGAG
GTCAGGAGTTTGAGACCAGCCTGGCCAACATGGTGAAACCCTGTCTCTACTAAAAATACAAAAAATTAGC
CAGGTGTTGTGGTGCACACCTGAAATCCCCGCTACTTGGGAGGCTGAGGCCCGAGAATCACTTGAACTCG
GGAGGCGGAGGTTGCAGTGGGCTGAGATCATGCCACTGCACTCCCACCCTGGGGGACAATAGCGAAACTT
CGTCTCAGAAAAAAAAAAAATTAAAAATAGCTGGGTGTGGTGGCATGCGCCTGTAGTCCCAGCTACTTGG
GAGGCTGAGGTCAGAGGATCACTTGAAGCCAGGAAGTCAATGCTGCAGTAAGCTATGATGGCATCAGTGC
ATTCCAGCCTGACCAATGGAACAGGACCCTGCCAAGAAAGAGAGAGAGAAAGGGAGGAAGGAACGAAAGG
AAGGAAGGAAGGAAAGAAAGGAAAGGAAGGAAAGAAAGGAAAGGAAGTAAGTAAGGATGGATGGATCCAG
GTGCTCTAAACCAGCAGTTCCCAAAGTTTTTGGCACCAGGGGCCAGTTTCATGGAAGACAATTTTTCCAC
GGGTGGCAGGGTGGTGGGCAGCGGGGATGGTTTTGGGATGAGTCAAGTGCATTACATTTATTGTGCACTT
TATTTCTATTATTATTACATTGTAATATATAATGAAAAAATTATATAGCTCACCATAATGTACAATCGGG
GGGTCCTGAGCTTGCTTTCCTGCAACTAGACAGTCCCATCTGGAGGTGCTGGAAGACAGTGACAGATCAT
CAGGCATTAGATTCTCATAAGGAGTATGCAACCTAGATCCCTCGTGTGCGCAGTTCCCAGGCATGAGCTC
AGGTGGTAGTAGTGCTCACTCACCTGCAGCTCTCCTCCTGCTGTGTGGCCCAGTTCCCAACAGTCCGTGG
ACCTGCACTAAAGGCTGTGGCCTGGTCTGCTGACCTCTCTCCAGAGCCCACGGTACTAGTCCATGGCCTG
GGAGTTGGGGACCCCTGCTCTAAGCAGTGAGGGAGCTGATTTGCAGACGGGGGAGTGGACACCGGGCAGC
TGGAGCCCTGGCTTTCTGCCACTGTCTCCTGGTGAAGGGCGGGCCTGGCTGGGAGGAGAGGGCCCCCCTC
CAGCCTCCCCAGGGGCATGATGCGCTGTGTGTCCCTGCTTCTAGATGCCGACAAAAGGATCAAGGTGGCG
AAGCCCGTGGTGGAGATGGATGGTGATGAGATGACCCGTATTATCTGGCAGTTCATCAAGGAGAAGGTAG
TGCCCCCTCCTGAAGTGGGTGGCTCTCCAGGTGGGCTGGCCAGGGATTGTTCTGTCCCACAGGGTCTTCT
GGACTGCAGGTCCCTAGGACCCCCCCGTTGTCCTGGTAGGCAGCAGCAGCTCTGTTTCTCTCCCTCTGTC
TACCCTTCTTTCACATCTGGTCCACTAATTGTTCAGAAGTCGTCAGGCGGGGGCCTGTCCCCAGCAGTCC
GTGTTGTGATGGTGCCAGATCCCACACGTTCTGTCAACTTTGAGAAGCTTCAAGGTTAATCCTGTATCAT
TTCCATCTTGATCCCCTACGTTTTGCCTCACTTCTTGAAAAGGAGTGAAAAGGGAACAGGGCTGGTGTGT
AGGGCAGTGACCCAGCCTGCAGTTGTGACGAGCCACCATGGCAAAGGGGTCCCAGAGTGGCCGCTTGGCT
TCTCCAAAGTTGCCGTGGTCTATGTATGTCCAGGAGAGCATGTAGTCCTTGTGAAGGCCCCACACTGTGT
GTGCGTTCATGGGACAAAGGTTGAGAACTGTCACTTCCAACTGTAAGATCTCAAAGCACTTGAGAAGAGG
AAACCAGTTGTATAGAGAAATCTAGATGTACTTGGGGTTGGGGTTTGGCTGAGTTGATGGGCCATGTGAG
GGGGGCACACAGGCACAGTGGAGGAAGAACCCTCCAAAAGACTGCGGCCTGGCCTGCCAACCTCTCTCCA
AAGCCCCAGCCTCTGCCAGCTCTGGCCAGGCCCCACTGAGAAATGGCTGACTCCAGCTTTCTGCTGCGCC
CCTCCACTGGCCTTCACTCATCCCTGTTTTGACTGACTGTCCCCTGCCATGGCCTGCAGACTTCTTATCC
TGCCCTTTGTTGTCATGTCCCTGAGTCACTGGGGTGACGCCTTGCTCTGGCCCTCTGTCCCCAGCTCATC
CTGCCCCACGTGGACATCCAGCTAAAGTATTTTGACCTCGGGCTCCCAAACCGTGACCAGACTGATGACC
AGGTCACCATTGACTCTGCACTGGCCACCCAGAAGTACAGTGTGGCTGTCAAGTGTGCCACCATCACCCC
TGATGAGGCCCGTGTGGAAGGTGCGAGGGTGTGGAGGTGGGCGGGCCAGGGAGGGTCACAGGCTTCTCCC
ACCGGCCTCTCCAGACTTGGGGTGGGGCAGTTGGCCATGTCACTCATCTCGGGACTGGGAGGTTGGGAGG
CTCCAGAGCTGGGGCTTTCCTGAGGCCAACTGCACCTGAGGCCAGTCTCCCTGTGTGTGCCTGGTGGACA
GTGGGGCCTCAGAGGTCACCTTTGATGAGGCTGGGGCAGGCGTGGGTAAGGGGCTGGCCTCAGGTTCCAC
GGTTAGGGCACAGCAGAGCTGAGAACAGAATCCATGTCTTCCATCTCCCAGGCCAGTGGTGTTTCTGCTT
CACTCTGGCTCTGACAGTGGTAGGTGTCATCGGTGACAGGGCTGAGGAGGAGGAGGAGGAGGAAGGTAAT
GCTGATGTGGTCACAGTAAGGATGGCTTCTTTGGGCAGTAGTCCATTCTGGCGTGGACAGTCTTGCAGGC
AGGTATGCAGAGAGATTGTGCAGAGGAGTGCTGTGTGTAAGGCCTGTGTCCTTCAGAGCCTCAGCAGCCT
CTGGGGAACATTGCTGTTGCCCTCCTGCCACTGGCTTGGCCTTGCAGGTCTGCCTCTGTCCCCATGGTCA
TGCAGGGTCACTGAGAGGTGCCCCCACCAGGCCTAGACTGGCTTCTGGTCCTTGGCTGTTGGGGCCGCTC
TCGAGGGTTCCATGAAGTCACAGCAGTGTGTGTGCAGAGGGGCCAGGTTTGGATAATTCGAGAAAGGATG
GTTGAGGAGTTTTCTGAGCTCTCTCTTCCTACTTTCCACAGTTGGAGGGAGCTGGCGGAAAGTACTCGAG
ATACATCTGCAAATTCCCCAGGCCTGCCCCTGTCCTGCAGGTCATGCCTGACCCACAGCACTGGGATCAT
TTGAGTGTTGAGCTCTTCATTTCACCTGAACCTTGGGAGTTGTGTATCCCCATTTATGGACAAGGCAGGA
GAGGGTCACAGCCTTAGGAACTGGGGTGGCGTTGGGGCCAGGACCGGAGCATGTGGAGTGGGCAGTGTGT
TGTCAGGCTTCGTCAGACAAGTCCTTCCCCCGGACTGTACTCCCTCCATGTGTGATGTGGGAATCATAAT
GGCCAACAGAGCACTTAGCATGGACCGGGCACCGTTCTAGGCCCTTTACACACGAGCTCATTTCGTCCTC
ACGACAACACTTAGGCTGGTTATTTTTCCAGTAATTTTTTTTTACAGCTTTGGTTGAGCCACTTTTGTAA
GCCTCACCATTAAAACTGGCAGACTTGCTGGGCATGGTGGCTCACACCTGTAATCCCAGCACTTTGGGAA
GCCGAGGCGGGTGGATCACCTGAGGTTGGGAGGTCGAGACCAGCCTGACCAACATGGAGAAACCCCATCT
CTCCTAAAAATACAAAATTAGCTTGGTGTGGTGTCACATGCCTGTAATTCCAGCTGCTCGGGAGGCTGAG
GCAGGAGAACCGCTTGAACCCAGGAGGCAGAGGTTGTGGTGAGCCGAGATCGCGCCATTGCACTCCAGCC
TGGGCAATAGGAGCAAAACTCTGTCTCAAAAAAAAAAAAAAAAAAAAAAAAATTGGCAGACTCCAGAGCC
CACACATTTGCACTCTAGACTCTACTGCCTTCCTCATGAAGAATTTTAGGACCCCCGTCTGGCTGTGTTG
TTGCTTGGGGTTCAAATTCTGGTTGAAAGATGGCGGCTGCAGTGGGACCACTATTATCTCTGTCCTCACA
GAGTTCAAGCTGAAGAAGATGTGGAAAAGTCCCAATGGAACTATCCGGAACATCCTGGGGGGGACTGTCT
TCCGGGAGCCCATCATCTGCAAAAACATCCCACGCCTAGTCCCTGGCTGGACCAAGCCCATCACCATTGG
CAGGCACGCCCATGGCGACCAGGTAGGCCAGGGTGGAGAGGGGATCCACTGACCTGGGCACCCCCCGACT
GGAGCTCCTCGCCTAGCCATCCTCTTGTCTCTGCAGTACAAGGCCACAGACTTTGTGGCAGACCGGGCCG
GCACTTTCAAAATGGTCTTCACCCCAAAAGATGGCAGTGGTGTCAAGGAGTGGGAAGTGTACAACTTCCC
CGCAGGCGGCGTGGGCATGGGCATGTACAACACCGACGAGGTGAGGCTGGCTTGGGCATCCTGGGCCCCT
CCTCTCCCAGCTTGTCTCTTCATCTCTGTCTCGTGGCTTTCCTTTCTTCTGTAATGGCCTCATTCTTAGG
CCACAAAGAGGCAGAAATGGCCACCAGGGCTGGGCATGGTGGCTGACACTTGTAATCCCGGCATTTTGGG
AGGCCAAGGCAGGATTGCTTGAGACCAGAAGTTTGAGACCAGCCTGGGCAACAGTGAGACCCCATCTCTT
GAAACAAAAAGAAAAGAAAAGAAAAGAAAAGAAATGGCCACGAGCAGCTCCAGGCGTACTCCTGCCTGCT
TGGCTACCCTATCAGAAGAGAATGCCTCTTTATCCCCAGTTCCAGCTCCAATGCCAGGCTGAGTCACATG
ACCACCTCTGAGCCAATCACAGAGGCCCTGGGGAGAAAGGCTTCTGATTGGATAAGCCAGGGACACACTG
TCAACCCCAGCGCTAGGCCTGGGAATTAGCCCTGCCCCAGCCACATGTAGTCTAAGGCGGTGCTTTCCAA
AAAAAAGTTGTTTCCAAAAAAAAAAAAAAAAAGGCGGTGTTTTCCAAAAAAAAAAAAAAGGCGGTGTTTT
ACAAAGCAAAGTTGAGAGGGAGAGGCTGGGCCAGCAGAAACATCGTGTGCACTGCACGGAGGCTGGTGTT
AAACAGTCGCGTGGGCGGCGGGGTACCGTTCCTGGAGAGCTGGGCCTTGCCCTGGGAGGTGGGAGGTTGC
CGGCAATCGCCAGGCTAGGGCACCACGCCAGGGCCCTGTCTCTCCCCCTGCAGTCCATCTCAGGTTTTGC
GCACAGCTGCTTCCAGTATGCCATCCAGAAGAAATGGCCGCTGTACATGAGCACCAAGAACACCATACTG
AAAGCCTACGATGGGCGTTTCAAGGACATCTTCCAGGAGATCTTTGACAAGTAAAGCCTCATCCATGTAC
TCTGTGGCCTTTCTTCCCTTCCCCCCATGCTGTTCCCATCCTACCCTGGGAAGGTCGCTATTAGAGTGCA
TTTGGCTCAGCTCCGAGGCTCAGGGAGGGATCCCCAACCTGTCAGCCTTCTGCCCTCTCCCCATAACAGA
CCTTTTTACTCCCAGGCACTATAAGACCGACTTCGACAAGAATAAGATCTGGTATGAGCACCGGCTCATT
GATGACATGGTGGCTCAGGTCCTCAAGTCTTCGGGTGGCTTTGTGTGGGCCTGCAAGAACTATGACGGAG
ATGTGCAGTCAGACATCCTGGCCCAGGGTACGCTGGGAGGACGCTGCTCCCTGTGGGGGTGGACTGTTGG
TCTTCTCTGGCTGGAGGGATTTTCAGACCCTTTTGGTAGAAAACTCTAGTGGAGCAGGACTCATTGCAGG
GTCCTCTGTGGACAGCAGGGGGCAGGAGCTCCCTGGCATCTGAGTGAGAGAGGATCACAAATTAGGTTTG
GACTGGTGTATGACTGAGGCTGCTCTTAGAGAGAAAGGAGGGCACAGGAATTCCAGGGCTAGGTTGACAG
CCCTAAGGTTGCTCCAGTCCATGATGGAGCCTCATGACTCTTTGGTTCCAGTAGGAAAATGGGATCAAGC
GGGACTGGAGGTTCTTTAAACTACAGCGAATGCATTTTGCATAGTGAACCTAATGGATGGAAGAGAATGT
TGGGGAAGCCTGCAAACCCCCAAGCCCTAGGGTGTCCTAAAAGACAAATCAGATTGCAGTAAAAAGTATA
GGTGATGAGGTCAGGAGTTTGAGACCAGCGTGGCCAACATGACGAAACCCCGTCTCTACTAAAAATGCAA
AAAAATTTAGCTGGGCATGGAGGCGGGCACAGTCATGGACACCTGTAATCCCAGCTACTTGAGAGGCTGA
GGCAGGAGAATCTCTTGAACCCAGGAGGTGGAGGTTGCAGTGAGCCGAGATTGCGCCATTGCACTCCAGC
CTGGGCAACAAGGGTGAAACTCCGTCTCCAAAAAAAATAAAAAATAAAAAATAAAGTATAGGTGAGACCA
TTAGAGAAGAACAGCGAGAAATGGGCCAATTGTGTTGGGAACAAGAGCAGGAAAGGTGGTAGGTGTGGGA
AACAGGCAGGACATAGAGAGTGAAAGAGAAACCTAAGCTTACCAGGGCAGCTCCTGGTATGGGGAAAAGG
GAAGCCCAGGACGGGTTCTGATGAAAAGCAGAGTCTTGTTACTTTGTGTAAACCTTTGGCTTTACTTTGG
AAAGTTCAGTGGGGATCCTCTCACCGAACAATTCCCATCTAGGATAGAAGGATGAAACGCCATAGCAGAT
GAGGCAGAGGCATGAAAATGAACCTTAAGTGTCTAAGGAATGGTGTGAAGGTGCAGGTAAAATGGAGAAG
GGATTGCTTACAGCCAAAATTCTTAGAATTCATAGACAAAGCATTTTCCTGTAGATGTTGTCCATGTTTT
CTTTCTTCAATCCTTATTTTGGTGTCATGGGGGTGCCTTTGAAGCCCTGGAGTCCTTGACAATAGAAAAG
CTTGCTGGAGTTGACATGTGGATTTGGGAGCATTCAGAAATTAATCAAAAAGCTTGAAAGTGGCCTAAGA
GAGTAGCTGCAAAAGTGATTGTTGGGAAATAGCTGACCACAGCTTTATTGTTTTCCTTGAATGTGCCTCC
GGGTCACGAGCCAGGCTGGGTGGCAGGCTGTGGGGGACCACAGGACTCTGTGGCCTCTTGGGCCCTGTGG
GCCACAGGCTGTGGGGCGCCTGGAGCTGGTGACACAGATGGATGTTAATGTGCTGGTGATAGCCACCCTC
CAGTGCCCTCCAGCCCTGTGCTGGGCCCTGGAGACCCACAGGAGGGTGAAGAGACCTGGAACAGTCCCTG
TCCTCCCAGTTGCAGCTGGGGGAGGCTGAGTAGAGCCACGAACTATGGCAGCTACAATATTGGGTTGTAG
AGGGCAGCAGGGCTCAGCTGGGTGGCCCCAGGAGAGGCGAGGCCCTGAGAGAAAGGCTTTCTACCCTCCA
GGCTTTGGCTCCCTTGGCCTGATGACGTCCGTCCTGGTCTGCCCTGATGGGAAGACGATTGAGGCTGAGG
CCGCTCATGGGACCGTCACCCGCCACTATCGGGAGCACCAGAAGGTGAGTGCAGGGCATGGGGCCTCACA
ATGCCCCTCTACCCCAGGGGCCTAAAGCCCACTGGGCTGAGGGCTGGAAGGACGAGCCTGACCCACCAGC
CCAGGGGGGGGCAGCTGTATCTGGGCAGCCTCCTCACTAGCTGGCTCGGCTCTTGATCTCCCTGCAACCC
CCGTCCCCAGGGCCGGCCCACCAGCACCAACCCCATCGCCAGCATCTTTGCCTGGACACGTGGCCTGGAG
CACCGGGGGAAGCTGGATGGGAACCAAGACCTCATCAGGTGAGCATGGAGGGAGAGGCCGGAGCTGCCCA
GGACAGGCTCTGGGTCCTGCCCTTGCACAGATGGGGTCTCATTCTGCCCCATCCCCATAGGTTTGCCCAG
ATGCTGGAGAAGGTGTGCGTGGAGACGGTGGAGAGTGGAGCCATGACCAAGGACCTGGCGGGCTGCATTC
ACGGCCTCAGCAAGTGCGTGGCCTGGGGTGGTAGGCAGACCCTGGGGCCATGTGGGAGGAAGCCAGGGGA
CCCCTCCTAAAGTCCCCCTCATGAGTCAGCTTAGCTGCAAGGGGGCCTGGGGCAGGACATGTCCACAGCC
CTGAGAGACTGCTCAGGCCAGCCTCTGTAGACCAGGTCTAGTGAGTTCCTCCCCATCCCTGAGGCTGGCT
ACTCAGAGCTCCTTCCGACCCTGCCCACACCCCTTTCCCAGCTTCTCTCAGGAGAGCAGTTACCTGGCAA
GACAGACCCTGCGGGTGCCTGCCGAATCTCGGTGGCCTCATGGTAGGGGGCTATAGCTGGCCTCTGGCCT
GTAGTCTGGGTGGCAGGAGATGGCTGTTCTGATGCCCAAGCTCAGGCTGTTCCCTGGAAGGGCCTGGCTG
ATGGCCTTGGCAAGGCCTGGAGCCACCCCTGATGTGAGTGGTGCTCTCTCTGCAGTGTGAAGCTGAACGA
GCACTTCCTGAACACCACGGACTTCCTCGACACCATCAAGAGCAACCTGGACAGAGCCCTGGGCAGGCAG
TAGGGGGAGGCGCCACCCATGGCTGCAGTGGAGGGGCCAGGGCTGAGCCGGCGGGTCCTCCTGAGCGCGG
CAGAGGGTGAGCCTCACAGCCCCTCTCTGGAGGCCTTTCTAGGGGATGTTTTTTTATAAGCCAGATGTTT
TTAAAAGCATATGTGTGTTTCCCCTCATGGTGACGTGAGGCAGGAGCAGTGCGTTTTACCTCAGCCAGTC
AGTATGTTTTGCATACTGTAATTTATATTGCCCTTGGAACACATGGTGCCATATTTAGCTACTAAAAAGC
TCTTCACAAAATTGTCTGCTGTGTTTGTCCCTGAGGGGAGGAGGTAGTGGGACCCTGAGGCAGAGGCCCT
GCTAGAGCTGGCAGGTTCCCCTGGGGCAGACCAGAGCACCTCAGGAAGGGGCTGCCACGGCAGGGAAGGG
ACCAGGCAGCCCTGGGAGCCCGCATTCCACAGGGGCCCACTGCGGAGTTCTCGGACACTCAGGGCACAGG
CCTGTGGGTTCCCTGGAATTTTCTAGCATGATCCAGTTTCTGTGTCCAGTTCTCCATTCTGAGAGTCAAT
CAGTTCCTGATAGGTTGTCATTGATTTTTTTCTTCGTTGGTTTTAACCTTCTAAACATCTCCAGGCCACT
TTCTTAGCCTTTTTCTAGGTACTAAAAAGAGGTCCTACCCACACCTGCCTCACACTTCTCCTTTCCAAGG
CTGCCTGAGTTTGGAGGGGCTTGGGTGTGTGTGAACAAGGGCCCTGCATTGTCTAGGCCTGCAGTTCCCA
GGCTTGGGTTCACTTTCACCATGCATTGGCAAAACTAGAAAAGTAAGCTTGTGACAAATTGTTCTCGGCC
GGGCACAGTGGCGCACGCCTATAATCCCTGTACTTTGGGAGGCTGAGGTGGGTGGATCACTTGAGGCCAG
GAGTTCGAGACCAGCCTGGCCAACATGGTGAAACCCCATCTCTACTCAAAATACAAAAATTAGCCAGGCG
TGGTGATGCGCACCTGCAGTCCCAGCTACTCGGGAGGCTGAGGCAGGAAAATGGCTTGAACCTGGGAGGC
AGAGGTTGCAGTGAGCCGAGACTGCACCACTGCACTCCAGCCTGGGTGACAGAGCAAGACTCTGTCTCAA
AAAAAAAAAAAAAAAAAAAAAAAAAAAAATTGCTCTATGCAAGTTTTTTAGCCCCATGTAAAAAGGAAAA
ATAAAAGTTGCTCTAAAGCTACATATAATCAAAA
SEQ ID NO: 40 (NM_002168.4, IDH2 isoform 1 mRNA):
AGCCCGCGGCCAGGCAGCCGGGAGGAGCGGCGCGCGCTCGGACCTCTCCCGCCCTGCTCGTTCGCTCTCC
AGCTTGGGATGGCCGGCTACCTGCGGGTCGTGCGCTCGCTCTGCAGAGCCTCAGGCTCGCGGCCGGCCTG
GGCGCCGGCGGCCCTGACAGCCCCCACCTCGCAAGAGCAGCCGCGGCGCCACTATGCCGACAAAAGGATC
AAGGTGGCGAAGCCCGTGGTGGAGATGGATGGTGATGAGATGACCCGTATTATCTGGCAGTTCATCAAGG
AGAAGCTCATCCTGCCCCACGTGGACATCCAGCTAAAGTATTTTGACCTCGGGCTCCCAAACCGTGACCA
GACTGATGACCAGGTCACCATTGACTCTGCACTGGCCACCCAGAAGTACAGTGTGGCTGTCAAGTGTGCC
ACCATCACCCCTGATGAGGCCCGTGTGGAAGAGTTCAAGCTGAAGAAGATGTGGAAAAGTCCCAATGGAA
CTATCCGGAACATCCTGGGGGGGACTGTCTTCCGGGAGCCCATCATCTGCAAAAACATCCCACGCCTAGT
CCCTGGCTGGACCAAGCCCATCACCATTGGCAGGCACGCCCATGGCGACCAGTACAAGGCCACAGACTTT
GTGGCAGACCGGGCCGGCACTTTCAAAATGGTCTTCACCCCAAAAGATGGCAGTGGTGTCAAGGAGTGGG
AAGTGTACAACTTCCCCGCAGGCGGCGTGGGCATGGGCATGTACAACACCGACGAGTCCATCTCAGGTTT
TGCGCACAGCTGCTTCCAGTATGCCATCCAGAAGAAATGGCCGCTGTACATGAGCACCAAGAACACCATA
CTGAAAGCCTACGATGGGCGTTTCAAGGACATCTTCCAGGAGATCTTTGACAAGCACTATAAGACCGACT
TCGACAAGAATAAGATCTGGTATGAGCACCGGCTCATTGATGACATGGTGGCTCAGGTCCTCAAGTCTTC
GGGTGGCTTTGTGTGGGCCTGCAAGAACTATGACGGAGATGTGCAGTCAGACATCCTGGCCCAGGGCTTT
GGCTCCCTTGGCCTGATGACGTCCGTCCTGGTCTGCCCTGATGGGAAGACGATTGAGGCTGAGGCCGCTC
ATGGGACCGTCACCCGCCACTATCGGGAGCACCAGAAGGGCCGGCCCACCAGCACCAACCCCATCGCCAG
CATCTTTGCCTGGACACGTGGCCTGGAGCACCGGGGGAAGCTGGATGGGAACCAAGACCTCATCAGGTTT
GCCCAGATGCTGGAGAAGGTGTGCGTGGAGACGGTGGAGAGTGGAGCCATGACCAAGGACCTGGCGGGCT
GCATTCACGGCCTCAGCAATGTGAAGCTGAACGAGCACTTCCTGAACACCACGGACTTCCTCGACACCAT
CAAGAGCAACCTGGACAGAGCCCTGGGCAGGCAGTAGGGGGAGGCGCCACCCATGGCTGCAGTGGAGGGG
CCAGGGCTGAGCCGGCGGGTCCTCCTGAGCGCGGCAGAGGGTGAGCCTCACAGCCCCTCTCTGGAGGCCT
TTCTAGGGGATGTTTTTTTATAAGCCAGATGTTTTTAAAAGCATATGTGTGTTTCCCCTCATGGTGACGT
GAGGCAGGAGCAGTGCGTTTTACCTCAGCCAGTCAGTATGTTTTGCATACTGTAATTTATATTGCCCTTG
GAACACATGGTGCCATATTTAGCTACTAAAAAGCTCTTCACAAAATTGTCTGCTGTGTTTGTCCCTGAGG
GGAGGAGGTAGTGGGACCCTGAGGCAGAGGCCCTGCTAGAGCTGGCAGGTTCCCCTGGGGCAGACCAGAG
CACCTCAGGAAGGGGCTGCCACGGCAGGGAAGGGACCAGGCAGCCCTGGGAGCCCGCATTCCACAGGGGC
CCACTGCGGAGTTCTCGGACACTCAGGGCACAGGCCTGTGGGTTCCCTGGAATTTTCTAGCATGATCCAG
TTTCTGTGTCCAGTTCTCCATTCTGAGAGTCAATCAGTTCCTGATAGGTTGTCATTGATTTTTTTCTTCG
TTGGTTTTAACCTTCTAAACATCTCCAGGCCACTTTCTTAGCCTTTTTCTAGGTACTAAAAAGAGGTCCT
ACCCACACCTGCCTCACACTTCTCCTTTCCAAGGCTGCCTGAGTTTGGAGGGGCTTGGGTGTGTGTGAAC
AAGGGCCCTGCATTGTCTAGGCCTGCAGTTCCCAGGCTTGGGTTCACTTTCACCATGCATTGGCAAAACT
AGAAAAGTAAGCTTGTGACAAATTGTTCTCGGCCGGGCACAGTGGCGCACGCCTATAATCCCTGTACTTT
GGGAGGCTGAGGTGGGTGGATCACTTGAGGCCAGGAGTTCGAGACCAGCCTGGCCAACATGGTGAAACCC
CATCTCTACTCAAAATACAAAAATTAGCCAGGCGTGGTGATGCGCACCTGCAGTCCCAGCTACTCGGGAG
GCTGAGGCAGGAAAATGGCTTGAACCTGGGAGGCAGAGGTTGCAGTGAGCCGAGACTGCACCACTGCACT
CCAGCCTGGGTGACAGAGCAAGACTCTGTCTCAAAAAAAAAAAAAAAAAAAAAAAAAAAAAAATTGCTCT
ATGCAAGTTTTTTAGCCCCATGTAAAAAGGAAAAATAAAAGTTGCTCTAAAGCTACATATAATCAAAA
SEQ ID NO: 41 (NM_001289910.1, IDH2 isoform 2 mRNA):
ATTTTGCAACGCCATAGGCTTCCAGCGACTGCTGGTGATGTTTCTGATGCCGACAAAAGGATCAAGGTGG
CGAAGCCCGTGGTGGAGATGGATGGTGATGAGATGACCCGTATTATCTGGCAGTTCATCAAGGAGAAGCT
CATCCTGCCCCACGTGGACATCCAGCTAAAGTATTTTGACCTCGGGCTCCCAAACCGTGACCAGACTGAT
GACCAGGTCACCATTGACTCTGCACTGGCCACCCAGAAGTACAGTGTGGCTGTCAAGTGTGCCACCATCA
CCCCTGATGAGGCCCGTGTGGAAGAGTTCAAGCTGAAGAAGATGTGGAAAAGTCCCAATGGAACTATCCG
GAACATCCTGGGGGGGACTGTCTTCCGGGAGCCCATCATCTGCAAAAACATCCCACGCCTAGTCCCTGGC
TGGACCAAGCCCATCACCATTGGCAGGCACGCCCATGGCGACCAGTACAAGGCCACAGACTTTGTGGCAG
ACCGGGCCGGCACTTTCAAAATGGTCTTCACCCCAAAAGATGGCAGTGGTGTCAAGGAGTGGGAAGTGTA
CAACTTCCCCGCAGGCGGCGTGGGCATGGGCATGTACAACACCGACGAGTCCATCTCAGGTTTTGCGCAC
AGCTGCTTCCAGTATGCCATCCAGAAGAAATGGCCGCTGTACATGAGCACCAAGAACACCATACTGAAAG
CCTACGATGGGCGTTTCAAGGACATCTTCCAGGAGATCTTTGACAAGCACTATAAGACCGACTTCGACAA
GAATAAGATCTGGTATGAGCACCGGCTCATTGATGACATGGTGGCTCAGGTCCTCAAGTCTTCGGGTGGC
TTTGTGTGGGCCTGCAAGAACTATGACGGAGATGTGCAGTCAGACATCCTGGCCCAGGGCTTTGGCTCCC
TTGGCCTGATGACGTCCGTCCTGGTCTGCCCTGATGGGAAGACGATTGAGGCTGAGGCCGCTCATGGGAC
CGTCACCCGCCACTATCGGGAGCACCAGAAGGGCCGGCCCACCAGCACCAACCCCATCGCCAGCATCTTT
GCCTGGACACGTGGCCTGGAGCACCGGGGGAAGCTGGATGGGAACCAAGACCTCATCAGGTTTGCCCAGA
TGCTGGAGAAGGTGTGCGTGGAGACGGTGGAGAGTGGAGCCATGACCAAGGACCTGGCGGGCTGCATTCA
CGGCCTCAGCAATGTGAAGCTGAACGAGCACTTCCTGAACACCACGGACTTCCTCGACACCATCAAGAGC
AACCTGGACAGAGCCCTGGGCAGGCAGTAGGGGGAGGCGCCACCCATGGCTGCAGTGGAGGGGCCAGGGC
TGAGCCGGCGGGTCCTCCTGAGCGCGGCAGAGGGTGAGCCTCACAGCCCCTCTCTGGAGGCCTTTCTAGG
GGATGTTTTTTTATAAGCCAGATGTTTTTAAAAGCATATGTGTGTTTCCCCTCATGGTGACGTGAGGCAG
GAGCAGTGCGTTTTACCTCAGCCAGTCAGTATGTTTTGCATACTGTAATTTATATTGCCCTTGGAACACA
TGGTGCCATATTTAGCTACTAAAAAGCTCTTCACAAAA
SEQ ID NO: 42 (NM_001290114.2, IDH2 isoform 3 mRNA).
AGCCCGCGGCCAGGCAGCCGGGAGGAGCGGCGCGCGCTCGGACCTCTCCCGCCCTGCTCGTTCGCTCTCC
AGCTTGGGATGGCCGGCTACCTGCGGGTCGTGCGCTCGCTCTGCAGAGCCTCAGGCTCGCGGCCGGCCTG
GGCGCCGGCGGCCCTGACAGCCCCCACCTCGCAAGAGCAGCCGCGGCGCCACTAGTTCAAGCTGAAGAAG
ATGTGGAAAAGTCCCAATGGAACTATCCGGAACATCCTGGGGGGGACTGTCTTCCGGGAGCCCATCATCT
GCAAAAACATCCCACGCCTAGTCCCTGGCTGGACCAAGCCCATCACCATTGGCAGGCACGCCCATGGCGA
CCAGTACAAGGCCACAGACTTTGTGGCAGACCGGGCCGGCACTTTCAAAATGGTCTTCACCCCAAAAGAT
GGCAGTGGTGTCAAGGAGTGGGAAGTGTACAACTTCCCCGCAGGCGGCGTGGGCATGGGCATGTACAACA
CCGACGAGTCCATCTCAGGTTTTGCGCACAGCTGCTTCCAGTATGCCATCCAGAAGAAATGGCCGCTGTA
CATGAGCACCAAGAACACCATACTGAAAGCCTACGATGGGCGTTTCAAGGACATCTTCCAGGAGATCTTT
GACAAGCACTATAAGACCGACTTCGACAAGAATAAGATCTGGTATGAGCACCGGCTCATTGATGACATGG
TGGCTCAGGTCCTCAAGTCTTCGGGTGGCTTTGTGTGGGCCTGCAAGAACTATGACGGAGATGTGCAGTC
AGACATCCTGGCCCAGGGCTTTGGCTCCCTTGGCCTGATGACGTCCGTCCTGGTCTGCCCTGATGGGAAG
ACGATTGAGGCTGAGGCCGCTCATGGGACCGTCACCCGCCACTATCGGGAGCACCAGAAGGGCCGGCCCA
CCAGCACCAACCCCATCGCCAGCATCTTTGCCTGGACACGTGGCCTGGAGCACCGGGGGAAGCTGGATGG
GAACCAAGACCTCATCAGGTTTGCCCAGATGCTGGAGAAGGTGTGCGTGGAGACGGTGGAGAGTGGAGCC
ATGACCAAGGACCTGGCGGGCTGCATTCACGGCCTCAGCAATGTGAAGCTGAACGAGCACTTCCTGAACA
CCACGGACTTCCTCGACACCATCAAGAGCAACCTGGACAGAGCCCTGGGCAGGCAGTAGGGGGAGGCGCC
ACCCATGGCTGCAGTGGAGGGGCCAGGGCTGAGCCGGCGGGTCCTCCTGAGCGCGGCAGAGGGTGAGCCT
CACAGCCCCTCTCTGGAGGCCTTTCTAGGGGATGTTTTTTTATAAGCCAGATGTTTTTAAAAGCATATGT
GTGTTTCCCCTCATGGTGACGTGAGGCAGGAGCAGTGCGTTTTACCTCAGCCAGTCAGTATGTTTTGCAT
ACTGTAATTTATATTGCCCTTGGAACACATGGTGCCATATTTAGCTACTAAAAAGCTCTTCACAAAATTG
TCTGCTGTGTTTGTCCCTGAGGGGAGGAGGTAGTGGGACCCTGAGGCAGAGGCCCTGCTAGAGCTGGCAG
GTTCCCCTGGGGCAGACCAGAGCACCTCAGGAAGGGGCTGCCACGGCAGGGAAGGGACCAGGCAGCCCTG
GGAGCCCGCATTCCACAGGGGCCCACTGCGGAGTTCTCGGACACTCAGGGCACAGGCCTGTGGGTTCCCT
GGAATTTTCTAGCATGATCCAGTTTCTGTGTCCAGTTCTCCATTCTGAGAGTCAATCAGTTCCTGATAGG
TTGTCATTGATTTTTTTCTTCGTTGGTTTTAACCTTCTAAACATCTCCAGGCCACTTTCTTAGCCTTTTT
CTAGGTACTAAAAAGAGGTCCTACCCACACCTGCCTCACACTTCTCCTTTCCAAGGCTGCCTGAGTTTGG
AGGGGCTTGGGTGTGTGTGAACAAGGGCCCTGCATTGTCTAGGCCTGCAGTTCCCAGGCTTGGGTTCACT
TTCACCATGCATTGGCAAAACTAGAAAAGTAAGCTTGTGACAAATTGTTCTCGGCCGGGCACAGTGGCGC
ACGCCTATAATCCCTGTACTTTGGGAGGCTGAGGTGGGTGGATCACTTGAGGCCAGGAGTTCGAGACCAG
CCTGGCCAACATGGTGAAACCCCATCTCTACTCAAAATACAAAAATTAGCCAGGCGTGGTGATGCGCACC
TGCAGTCCCAGCTACTCGGGAGGCTGAGGCAGGAAAATGGCTTGAACCTGGGAGGCAGAGGTTGCAGTGA
GCCGAGACTGCACCACTGCACTCCAGCCTGGGTGACAGAGCAAGACTCTGTCTCAAAAAAAAAAAAAAAA
AAAAAAAAAAAAAAATTGCTCTATGCAAGTTTTTTAGCCCCATGTAAAAAGGAAAAATAAAAGTTGCTCT
AAAGCTACATATAATCAAAA

What is claimed is:

1. A method for treating a clonal hematopoietic disease or disorder, the method comprising administering an inhibitor of a member of the nuclear receptor 4A (NR4A) family.

2. The method of claim 1, wherein the inhibitor binds with the NR4A family member or with a nucleic acid encoding the NR4A family member.

3. The method of claim 1 or 2, wherein the NR4A family member is selected from the group consisting of NR4A1, NR4A2 and NR4A3.

4. The method of any one of claims 1-3, wherein the NR4A family member is NR4A1.

5. The method of any one of claims 1-4, wherein the inhibitor is a nucleic acid.

6. The method of claim 5, wherein the nucleic acid is selected from the group consisting of siRNAs, antisense oligonucleotides, aptamers, ribozymes, and triplex forming oligonucleotides.

7. The method of claim 5 or 6, wherein the inhibitor comprises a nucleotide sequence substantially complementary to at least a portion of a nucleic acid encoding the NR4A family member.

8. The method of any one of claims 5-7, wherein the inhibitor comprises a nucleotide sequence substantially complementary to at least 15 contiguous nucleotides of SEQ ID NO: 1 (NR4A1 sequence), SEQ ID NO: 6 (NR4A2 sequence) or SEQ ID NO: 9 (NR4A3 sequence).

9. The method of any one of claims 1-4, wherein the inhibitor is an antibody or antigen binding fragment thereof that binds the NR4A family member.

10. The method of claim 9, wherein the antibody is an anti-NR4A1 antibody, an anti-NR4A2 antibody, an anti-NR4A3 antibody, or antigen binding fragment thereof

11. The method of claim 9 or 10, wherein the antibody is a monoclonal antibody.

12. The method of any one of claims 9-11, wherein the antibody is a humanized antibody.

13. The method of any one of claims 1-4, wherein the inhibitor is a small molecule.

14. The method of claim 13, wherein the inhibitor is a 1,1-bis(3′-indolyl)-1-(p-substituted phenyl)methane (C-DIM) compound.

15. The method of claim 13 or 14, wherein the inhibitor is 1,1-bis(3′-indolyl)-1-(p-hydroxyphenyl)methane (DIM-C-pPhOH), 1,1-bis(3′-indolyl)-1-(3-chloro-4-hydroxy-5-methoxyphenyl)methane (DIM-C-pPhOh-3-Cl-5-OCH₃), 1,1-bis(3′-indolyl)-1-(3,5-dibromo-4-hydroxyphenyl)methane (DIM-C-pPhOH-3,5-Br2), camptothecin (CPT), or a cyclooxygenase (COX)-2 inhibitor (celecoxib analogue SC-236).

16. The method of any one of claims 1-15, wherein the clonal hematopoietic disease or disorder is clonal hematopoiesis, myelodysplastic syndromes (MDS) or leukemia.

17. The method of any one of claims 1-16, wherein the clonal hematopoietic disease or disorder is leukemia.

18. The method of claim 17, wherein the leukemia is acute myelogenous leukemia (AML) or chronic myeloid leukemia (CML).

19. The method of any one of claims 1-16, the clonal hematopoietic disease or disorder is MDS.

20. The method of claim 19, wherein the MDS is MDS with multilineage dysplasia (MDS-MLD), MDS with single lineage dysplasia (MDS-SLD), MDA with ring sideroblasts (MDS-RS), MDS with excess blasts (MDS-EB), MDS with isolated del(5q), and/or MDS unclassifiable (MDS-U).

21. The method of any one of claims 1-20, further comprising co-administering an immunomodulatory agent to the subject.

22. The method of any one of claims 1-20, further comprising co-administering an anti-inflammatory agent to the subject.

23. The method of any one of claims 1-20, wherein the subject comprises at least one mutation in a nucleic acid encoding ASXL transcriptional regulator 1 (ASXL1), DNA (cytosine-5)-methyltransferase 3A (DNMT3A), isocitrate dehydrogenase (NADP(+)) 1(IDH1) or isocitrate dehydrogenase (NADP(+)) 2 (IDH 2).

24. The method of claim 16, further comprising, prior to onset of the treatment regime, identifying the subject with at least one mutation in a nucleic acid encoding ASXL1, DNMT3A, IDH1 or IDH 2.

25. The method of any one of claims 1-24, further comprising a step of assaying a sample from the subject for at least one mutation in a nucleic acid encoding ASXL1, DNMT3A, IDH1 or IDH 2 prior to onset of the treatment regime.

26. The method of any one of claims 1-25, wherein the subject is a mammal.

27. The method of any one of claims 1-26, wherein the subject is human.
